(19) United States
(12) Patent Application Publication
(10) Pub. No.: US 2024/0025918 A1
(43) Pub. Date: Jan. 25, 2024

(54) KRAS G12D Inhibitors

(71) Applicant: Jacobio Pharmaceuticals Co., Ltd, Beijing (CN)

(72) Inventors: Huifeng HAN, Beijing (CN); Panliang GAO, Beijing (CN); Wenlong ZHANG, Beijing (CN); Cunbo MA, Beijing (CN); Peng WANG, Beijing (CN); Dan LIU, Beijing (CN); Hao ZHANG, Beijing (CN); Wei LONG, Beijing (CN)

(21) Appl. No.: 18/037,809

(22) PCT Filed: Nov. 19, 2021
(86) PCT No.: PCT/CN2021/131660

§ 371 Date: May 19, 2023

(30) Foreign Application Priority Data
Nov. 20, 2020 (WO) PCT/CN2020/130351
Apr. 30, 2021 (WO) PCT/CN2021/091540
May 8, 2021 (WO) PCT/CN2021/092466
Jun. 9, 2021 (WO) PCT/CN2021/099242
Jun. 15, 2021 (WO) PCT/CN2021/100130
Jun. 24, 2021 (WO) PCT/CN2021/102172
Jun. 24, 2021 (WO) PCT/CN2021/102472
Sep. 30, 2021 (WO) PCT/CN2021/122046

(51) Int. Cl. C07D 519/00; C07D 487/08

(52) U.S. Cl. CPC C07D 519/00; C07D 487/08

(57) ABSTRACT

Provided are KRAS G12D inhibitors of formula (I), a composition containing the inhibitors, a prodrug thereof, a PROTAC compound thereof and the use thereof.

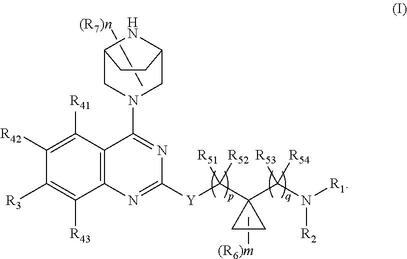

### CROSS-REFERENCES TO RELATED APPLICATIONS

This application claims priority to PCT/CN2020/130351, filed Nov. 20, 2020; PCT/CN2021/091540, filed Apr. 30, 2021; PCT/CN2021/092466, filed May 8, 2021; PCT/CN2021/099242, filed Jun. 9, 2021; PCT/CN2021/100130, filed Jun. 15, 2021; PCT/CN2021/102172, filed Jun. 24, 2021; and PCT/CN2021/122046, filed Sep. 30, 2021, all of which are incorporated herein in their entireties.

### TECHNICAL FIELD

The invention relates to KRAS G12D (glycine12 is mutated to aspartic acid) inhibitors, a composition containing the inhibitors, a prodrug thereof, a PROTAC thereof and the use thereof.

### BACKGROUND ART

RAS represents a population of 189 amino acid monomeric globular proteins (21 kDa molecular weight) that are associated with the plasma membrane and bind to GDP or GTP, and RAS acts as a molecular switch. When the RAS contains bound GDP, it is in a stationary or closed position and is inactive. When cells are exposed to certain growth-promoting stimuli, RAS is induced to exchange their bound GDP for GTP. In the case of binding to GTP, RAS is open and is capable of interacting with other proteins (its “downstream targets”) and activating the proteins. The RAS protein itself has an inherently low ability to hydrolyze GTP back to GDP, thereby turning itself into a closed state. Closing RAS requires an exogenous protein called GTPase activating protein (GAP) that interacts with RAS and greatly accelerates the conversion of GTP to GDP. Any mutation in RAS that affects its ability to interact with GAP or convert GTP back to GDP will result in prolonged protein activation, and thus conduction to the cell to inform its signaling of continued growth and division. Since these signals cause cell growth and division, over-activated RAS signaling can ultimately lead to cancer.

Structurally, the RAS protein contains a G domain responsible for the enzymatic activity of RAS, guanine nucleotide binding and hydrolysis (GTPase reaction). It also contains a C-terminal extension called the CAAX cassette, which can be post-translationally modified and responsible for targeting the protein to the membrane. The G domain contains a phosphate binding ring (P-ring). The P-loop represents a pocket of a binding nucleotide in a protein, and this is a rigid portion of a domain with conserved amino acid residues necessary for nucleotide binding and hydrolysis (glycine 12, threonine 26 and lysine 16). The G domain also contains a so-called switch I region (residues 30-40) and a switch II region (residues 60-76), both of which are dynamic parts of the protein, since the dynamic portion is converted between stationary and loaded states. The ability is often expressed as a “spring loaded” mechanism. The primary interaction is the hydrogen bond formed by threonine-35 and glycine-60 with the gamma-phosphate of GTP, which maintains the active conformation of the switch I region and the switch II region, respectively. After hydrolysis of GTP and release of phosphate, the two relax into an inactive GDP conformation.

The most notable members of the RAS subfamily are HRAS, KRAS and NRAS, which are primarily involved in many types of cancer. Mutation of any of the three major isoforms of the RAS gene (HRAS, NRAS or KRAS) is one of the most common events in human tumor formation. Approximately 30% of all tumors in human tumors were found to carry some mutations in the RAS gene. It is worth noting that KRAS mutations were detected in 25%-30% of tumors. In contrast, the rate of carcinogenic mutations in NRAS and HRAS family members was much lower (8% and 3%, respectively). The most common KRAS mutations were found at residues G12 and G13 in the P-loop as well as at residue Q61.

With respect to the KRAS G12C inhibitors, some progresses have been taken recently after many years of efforts, for example some promising clinical data have been reported when using Amg-510 and MRT-849 as the therapeutic agent. However, the development of KRAS G12D inhibitors is extraordinarily hard. Thus, there remains a need in the art for improved compounds and methods for treating KRAS G12D mutated cancer. The present invention fulfills this need and provides other related advantages.

### SUMMARY OF INVENTION

In one aspect, provided herein are the following aspects:

[1]. A compound of formula (I), a stereoisomer thereof, an atropisomer thereof, a deuterated derivative thereof, a tautomer thereof, a prodrug thereof, or a pharmaceutically acceptable salt thereof:

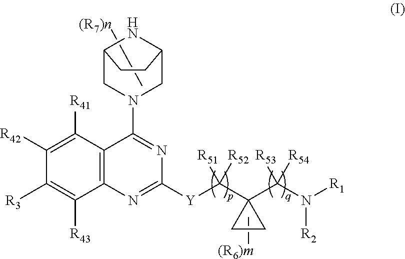

Wherein,
Y is selected from a bond, O, NR₅₅, S, S═O, or S(═O)₂;
R₁ and R₂ together with the nitrogen atom to which they are both attached form 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-20 membered monocyclic heterocyclic ring; said 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-20 membered monocyclic heterocyclic ring optionally further contains ring members selected from —O—, —S—, —S(═O)—, —S(═O)₂—, —C(═O)—, —NH—, —CH₂—, —CHF—, —CF₂—, —C(═O)NH—, —NHC(═O)—, —S(═O)NH—, —NHS(═O)—, —S(═O)₂NH— or —NHS(═O)₂—; said 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-15 membered monocyclic heterocyclic is independently optionally substituted with one or more R_{S};
R_{S} at each occurrence is independently selected from halogen —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein said —C₁₋₆alkyl, —C₁₋₆haloalkyl, —C₁₋₆haloalkoxy, —C₂₋₆alkenyl, —C₂₋₆alkynyl, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₃ is selected from phenyl, naphthyl, 5 membered heteroaryl, 6 membered heteroaryl, 8 membered heteroaryl, 9 membered heteroaryl or 10 membered heteroaryl; each of which is independently optionally substituted with one or more R₃₁;
R₃₁ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₄₁, R₄₂ or R₄₃ at each occurrence is independently selected from hydrogen, halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₅₁, R₅₂, R₅₃, R₅₄ or R₅₅ at each occurrence is independently selected from hydrogen, halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₆ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₇ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
m, n, p or q is independently selected from 0, 1, 2, 3, 4, 5, 6, 7, 8, 9 or 10;
said heterocyclyl, heterocyclic, or heteroaryl at each occurrence contains 1, 2, 3, 4, or 5 ring members selected from N, O, S, S(═O) or S(═O)₂.

[2]. The compound according to [1], wherein, the compound of formula (I) is selected from any one of formula (I-A) to formula (I-F):

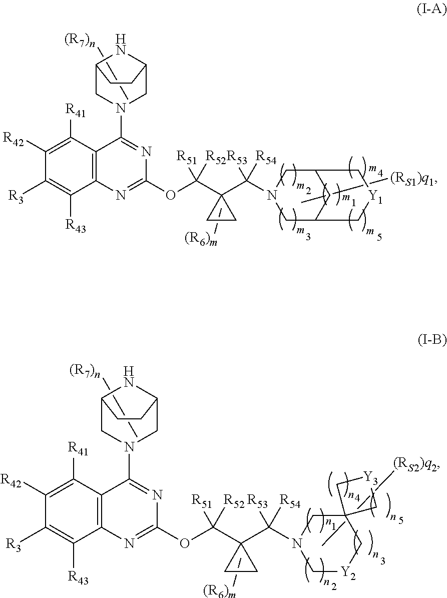

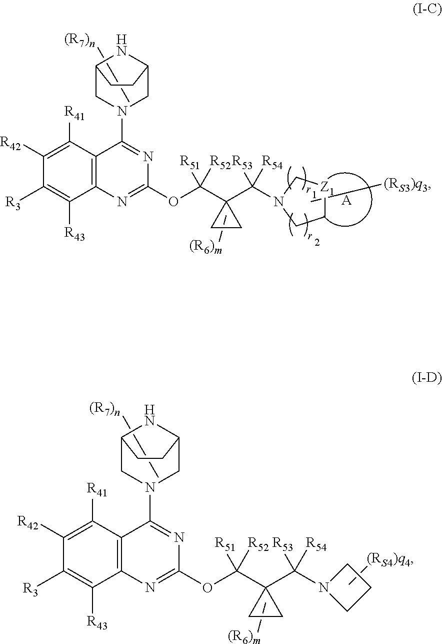

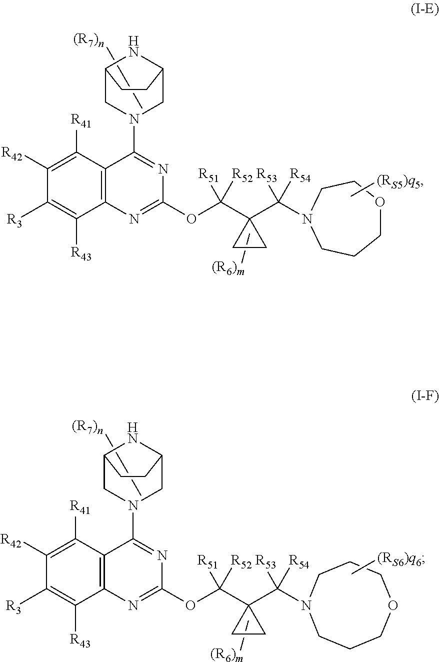

Y₁ is selected from —O—, —S—, —S(═O)—, —S(═O)₂—, —C(═O)—, —NH—, —CH₂—, —CHF— or —CF₂—;
m₁, m₂, m₃, m₄ or m₅ is independently selected from 0, 1, 2, 3, 4, 5 or 6;
Y₂ is selected from —O—, —S—, —S(═O)—, —S(═O)₂—, —C(═O)—, —NH—, —CH₂—, —CHF— or —CF₂—;
Y₃ are each independently selected from —O—, —S—, —S(═O)—, —S(═O)₂—, —C(═O)—, —NH—, —CH₂—, —CHF—, —CF₂—, —C(═O)NH—, —NHC(═O)—, —S(═O)NH—, —NHS(═O)—, —S(═O)₂NH— or —NHS(═O)₂—;
n₁, n₂, n₃, n₄ or n₅ is independently selected from 0, 1, 2, 3, 4, 5 or 6;
Ring A is selected from a 3-7 membered carbocyclic ring; 3-7 membered heterocyclic including 1, 2 or 3 ring members selected from —O—, —S—, —S(═O)—, —S(═O)₂—, —C(═O), —NH—, —CH₂—, —CHF— or —CF₂—; phenyl ring; or a 5-6 membered heteroaryl ring including 1, 2 or 3 ring members selected from N, O or S;
Z₁ is selected from C, CH or N;
r₁ or r₂ is independently selected from 0, 1, 2, 3, 4, 5 or 6;
R_{S1}, R_{S2}, R_{S3}, R_{S4}, R_{S5} or R_{S6} at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein said —C₁₋₆alkyl, —C₁₋₆haloalkyl, —C₁₋₆haloalkoxy, —C₂₋₆alkenyl, —C₂₋₆alkynyl, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
q₁, q₂, q₃, q₄, q₅, or q₆ at each occurrence is independently selected from 0, 1, 2, 3, 4, 5, 6, 7, 8, 9 or 10;
said heterocyclyl, heterocyclic, or heteroaryl at each occurrence contains 1, 2, 3, 4, or 5 ring members selected from N, O, S, S(═O) or S(═O)₂.

[3]. The compound according to [1], or [2], wherein, the compound of formula (I) is selected from formula (I-A):

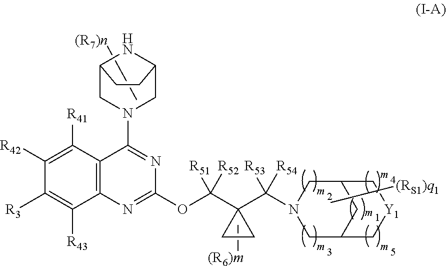

wherein,
Y₁ is selected from —O—, —NH—, —C(═O)—, —CH₂—, —CHF— or —CF₂—;
R_{S1} at each occurrence is independently selected from —F, —Cl, —Br, —C₁₋₃alkyl, —C₁₋₃haloalkyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —CHO, —C(═O)OH, —C(═O)(OC₁₋₃alkyl), —OC(═O)(C₁₋₃alkyl), —C(═O)NH₂, —C(═O)NH(C₁₋₃alkyl), —NHC(═O)(C₁₋₃alkyl), —S(═O)NH₂, —S(═O)NH(C₁₋₃alkyl), —NHS(═O)(C₁₋₃alkyl), —S(═O)₂NH₂, —S(═O)₂NH(C₁₋₃alkyl), —NHS(═O)₂(C₁₋₃alkyl), 3-6 membered cycloalkyl, 3-6 membered heterocyclyl, 6-10 membered aryl or 5-10 membered heteroaryl; said —C₁₋₃alkyl, —C₁₋₃haloalkyl, —NH₂, —OH, —SH, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —CH₃, —CH₂CH₃, —CH₂CH₂CH₃, —CH(CH₃)₂, —CH₂F, —CHF₂, —CF₃, oxo, —CN, —COOH, —NH₂, —NH(CH₃), —NH—CH(CH₃)₂, —N(CH₃)₂, —OH, —O(CH₃), —O—CH₂CH₃, —O—CH₂CH₂CH₃, —O—CH(CH₃)₂, —SH, —S(CH₃), —C(═O)—CH₃, —C(═O)—CH₂CH₃, —C(═O)—CF₃, or 3 membered cycloalkyl; preferably, R_{S1} at each occurrence is independently selected from —F, —Cl, —Br, —CH₃, —CH₂CH₃, —CH₂CH₂CH₃, —CH(CH₃)₂, —CH₂F, —CHF₂, —CF₃, —CH₂CH₂F, —CH₂CHF₂, —CH₂CF₃, —CHFCH₃, —CF₂CH₃, —CN, oxo, —NH₂, —NH(CH₃), —N(CH₃)₂, —OH, —O—CH₃, —SH, —S—CH₃, —S(═O)—CH₃, —S(═O)₂—CH₃, —CHO, —C(═O)CH₃, —COOH, —COOCH₃, —C(═O)NH₂, —C(═O)NH(CH₃), —NH(CHO), —NH—C(═O)CH₃, —S(═O)NH₂, —S(═O)NH(CH₃), —NHS(═O)CH₃, —S(═O)₂NH₂, —S(═O)₂NH(CH₃), —NH—S(═O)₂CH₃, —CH₂OH, —CH₂O(CH₃), —CH₂NH₂ or —CH₂NH(CH₃); more preferably, R_{S1} at each occurrence is independently selected from —F, —CH₃, —CN, oxo, —NH₂, —OH, —OCH₃, —COOH, —C(═O)CH₃, —S(═O)₂CH₃, —CH₂NH₂ or —CH₂OH;
q₁ is selected from 0, 1, 2, 3, 4, 5 or 6; preferably, q₁ is selected from 0, 1, 2, or 3.

[4]. The compound according to any one of [1] to [3], wherein, the compound of formula (I-A) is selected from any one of formula (I-A1) to formula (I-A6):

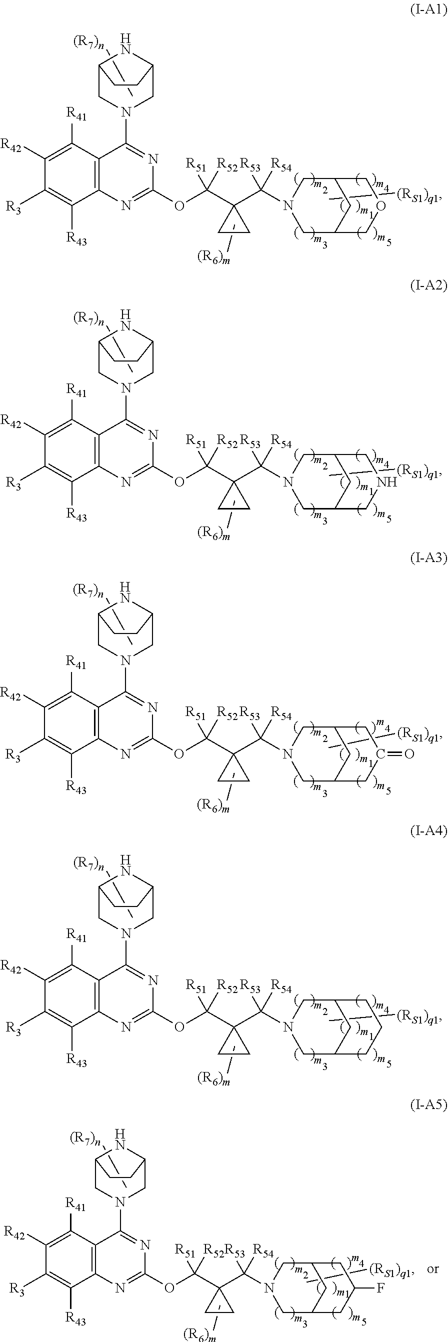

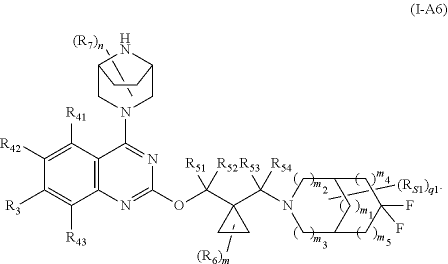

[5]. The compound according to any one of [1] to [4], wherein, the compound of formula (I-A) is selected from formula (I-A1):

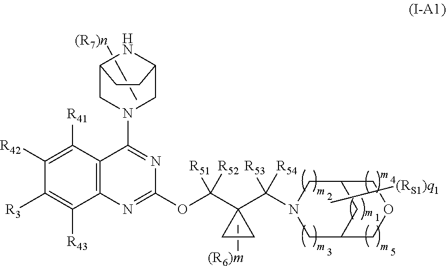

wherein, m₁ is selected from 1, 2 or 3; m₂ is selected from 0 or 1; m₃ is selected from 0 or 1; m₄ is selected from 0 or 1; m₅ is selected from 0 or 1.

[6]. The compound according to [5], wherein, the moiety of

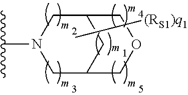

in the compound of formula (I-A1) is selected from

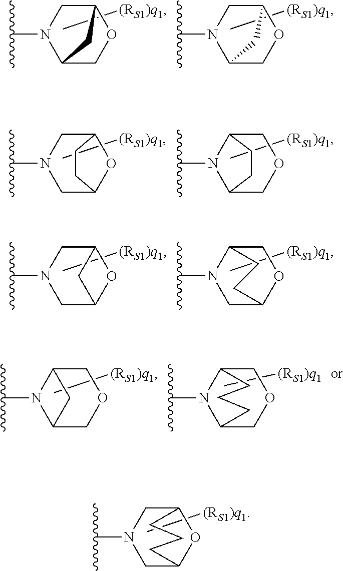

[7]. The compound according to any one of [5] to [6], wherein, the moiety of

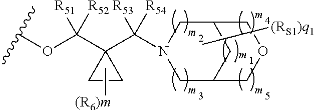

in the formula (I-A1) is selected from

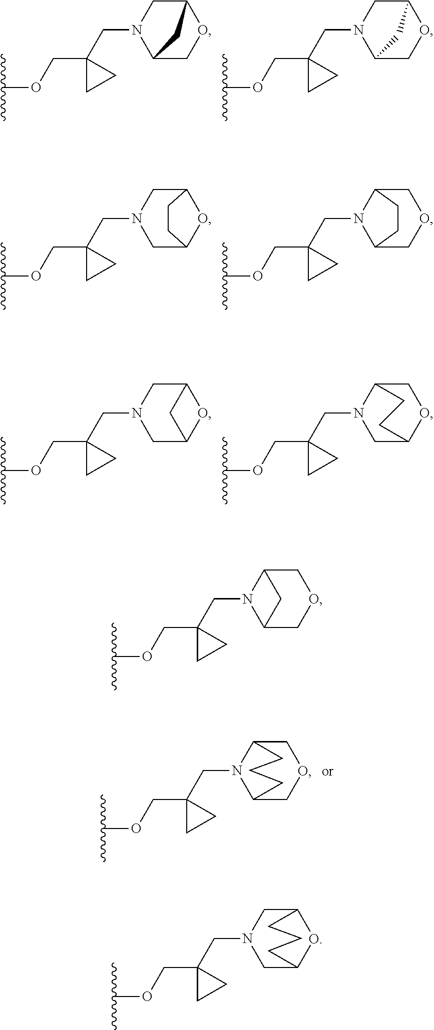

[8]. The compound according to any one of [1] to [4], wherein, the compound of formula (I-A) is selected from formula (I-A2):

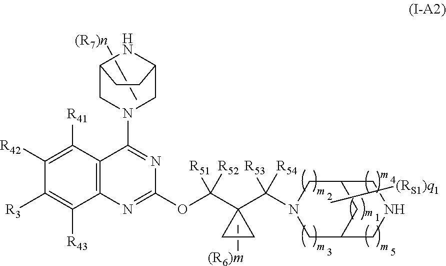

wherein, m₁ is selected from 1 or 2; m₂ is selected from 0 or 1; m₃ is selected from 0 or 1; m₄ is selected from 0 or 1; m₅ is selected from 0 or 1;
R_{S1} at each occurrence is independently selected from —C₁₋₃alkyl, —S(═O)(C₁₋₃alkyl), —S(═O)₂(C₁₋₃alkyl), —C(═O)(C₁₋₃alkyl); preferably, R_{S1} at each occurrence is independently selected from —CH₃, —CH₂CH₃, —CH₂CH₂CH₃, —CH(CH₃)₂, —S(═O)CH₃, —S(═O)₂CH₃, or —C(═O)CH₃.

[9]. The compound according to [8], wherein, the moiety of

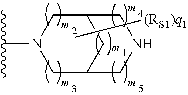

in the compound of formula (I-A2) is selected from

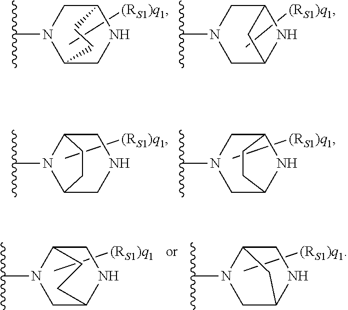

[10]. The compound according to any one of [8] to [9], wherein, the moiety of

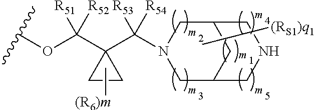

in the formula (I-A2) is selected from

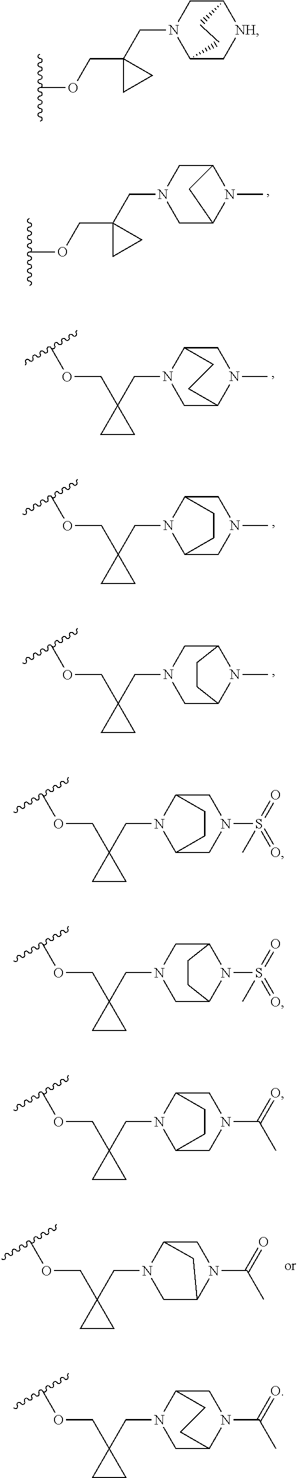

[11]. The compound according to any one of [1] to [4], wherein, the compound of formula (I-A) is selected from formula (I-A3):

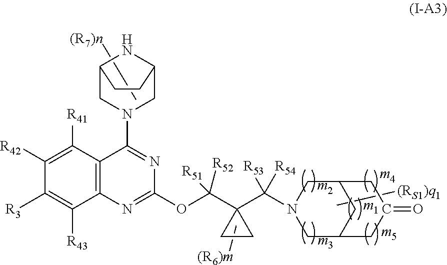

wherein, m₁ is selected from 1 or 2; m₂ is selected from 0 or 1; m₃ is selected from 0 or 1; m₄ is selected from 0 or 1; m₅ is selected from 0 or 1.

[12]. The compound according to [11], wherein, the moiety of

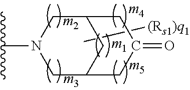

in formula (I-A3) is selected from

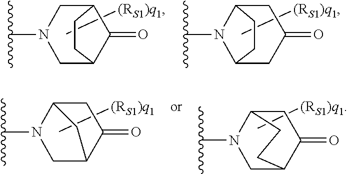

[13]. The compound according to any one of [11] to [12], wherein, the moiety of

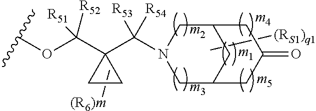

in the formula (I-A3) is selected from

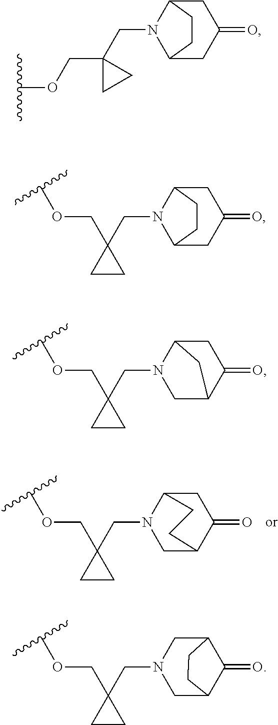 [SMILES: *OCC1(CN2C3CCC2CC(=O)C3)CC1 ; *OCC1(CN2C3CCC2CC(=O)C3)CC1 ; *OCC1(CN2CC3CC2CC3=O)CC1 ; *OCC1(CN2CC3CCC2CC3=O)CC1 ; *OCC1(CN2CC3CCC(C2)C3=O)CC1]

[14]. The compound according to any one of [1] to [4], wherein, the compound of formula (I-A) is selected from formula (I-A4):

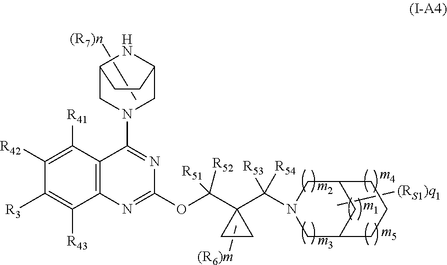

wherein, m₁ is selected from 1, 2 or 3; m₂ is selected from 0 or 1; m₃ is selected from 0 or 1; m₄ is selected from 0 or 1; m₅ is selected from 0 or 1;
R_{S1} at each occurrence is independently selected from —F, —Cl, —Br, —C₁₋₃alkyl, —CN, —NH₂, —OH, —O(C₁₋₃alkyl), —COOH or —C(═O)(OC₁₋₃alkyl); said —C₁₋₃alkyl is independently optionally substituted with 1, 2, or 3 substituents selected from —F, —Cl, —Br, —CH₃, —CH₂CH₃, —CH₂CH₂CH₃, —CH(CH₃)₂, —NH₂, —NH(CH₃), —N(CH₃)₂, —OH, —O(CH₃), —O—CH(CH₃)₂, or 3 membered cycloalkyl; preferably, R_{S1} at each occurrence is independently selected from —F, —Cl, —CH₃, —CH₂CH₃, —CH₂CH₂CH₃, —CH(CH₃)₂, —CN, —NH₂, —NH(CH₃), —N(CH₃)₂, —OH, —O—CH₃, —COOH, —COOCH₃, —CH₂OH, —CH₂O(CH₃), —CH₂NH₂ or —CH₂NH(CH₃); more preferably, R_{S1} at each occurrence is independently selected from —F, —CH₃, —CN, —NH₂, —OH, —OCH₃, —COOH, —C(═O)CH₃, —CH₂NH₂ or —CH₂OH;
q₁ is selected from 0, 1, or 2.

[15]. The compound according to [14], wherein, the moiety of

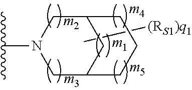

in the formula (I-A4) is selected from

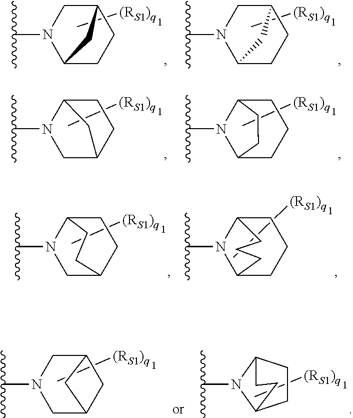

[16]. The compound according to any one of [14] to [15], wherein, the moiety of

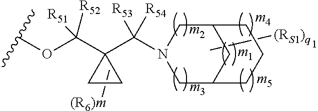

in the formula (I-A4) is selected from

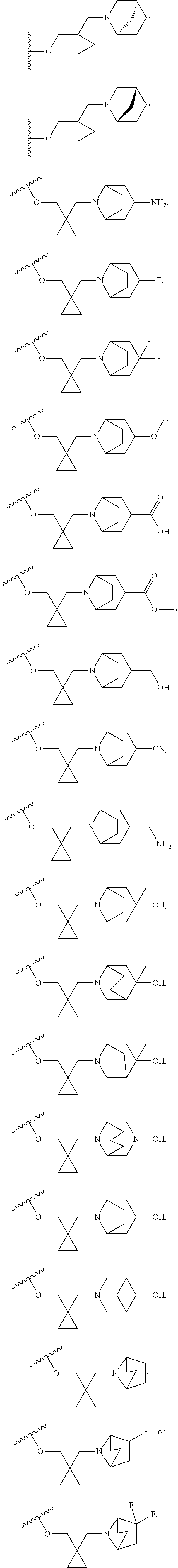

[17]. The compound according to any one of [1] to [4], wherein, the compound of formula (I-A) is selected from formula (I-A5):

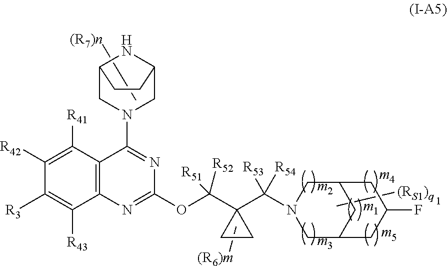

wherein, m₁ is selected from 2; m₂ is selected from 0; m₃ is selected from 0; m₄ is selected from 0; m₅ is selected from 1.

[18]. The compound according to [17], wherein, the moiety of

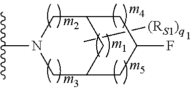

in the formula (I-A5) is selected from

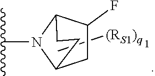

[19]. The compound according to any one of [17] to [18], wherein, the moiety of

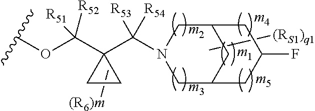

in the formula (I-A5) is selected from

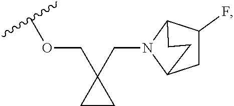 [SMILES: *OCC1(CN2C3CCC2C(F)C3)CC1]

[20]. The compound according to any one of [1] to [4], wherein, the compound of formula (I-A) is selected from formula (I-A6):

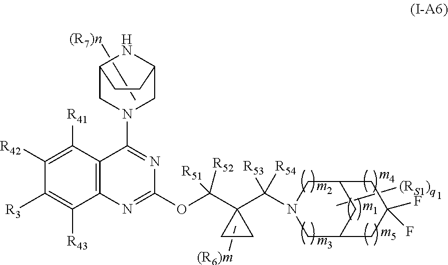

wherein, m₁ is selected from 2; m₂ is selected from 0; m₃ is selected from 0; m₄ is selected from 0; m₅ is selected from 1.

[21]. The compound according to [20], wherein, the moiety of

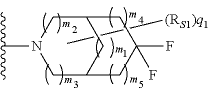

in the compound of formula (I-A6) is selected from

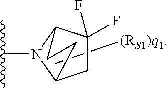

[22]. The compound according to any one of [20] to [21], wherein, the moiety of

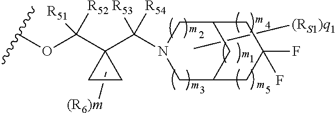

in the formula (I-A6) is selected from

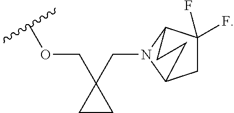 [SMILES: *OCC1(CN2C3CCC2C(F)(F)C3)CC1]

[23]. The compound according to any one of [1] to [22], wherein, the moiety of

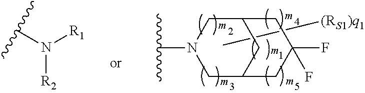

is selected from

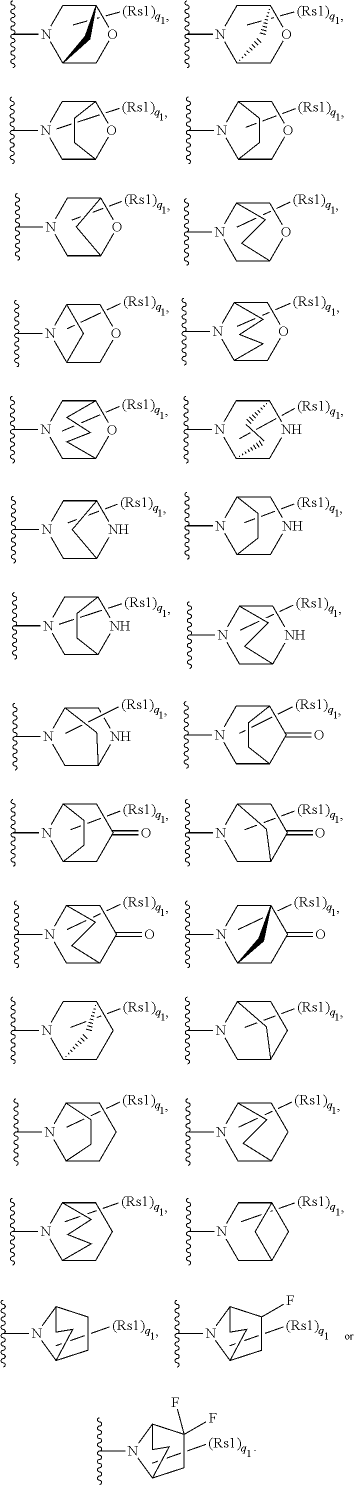

[24]. The compound according to any one of [1] to [23], wherein, the moiety of

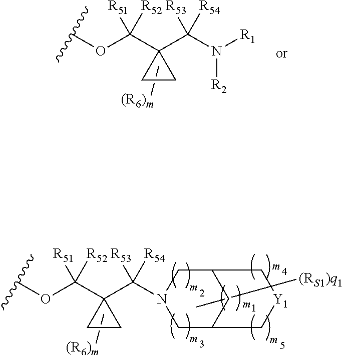

is selected from

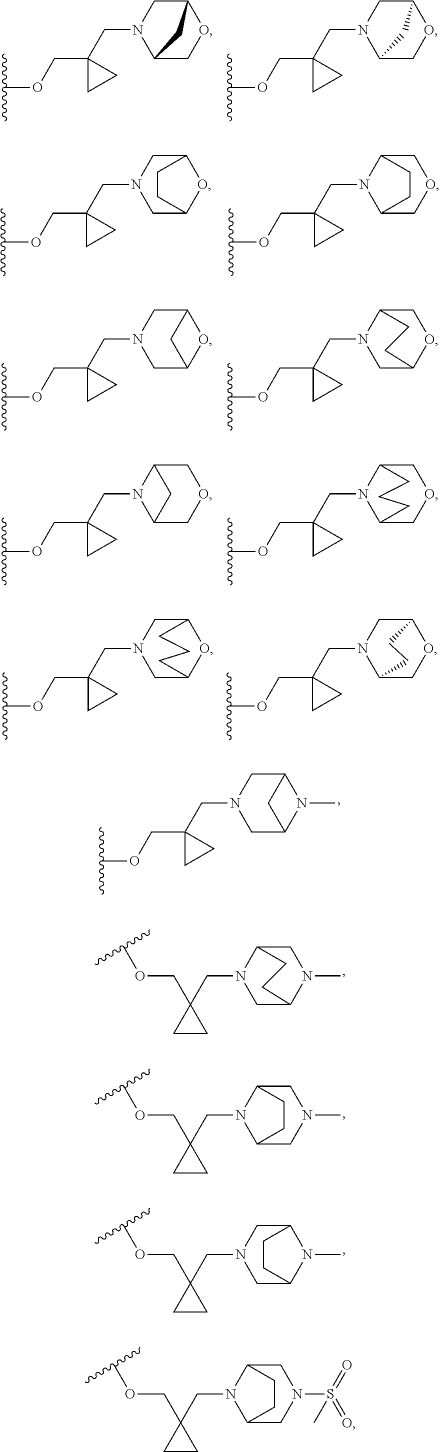

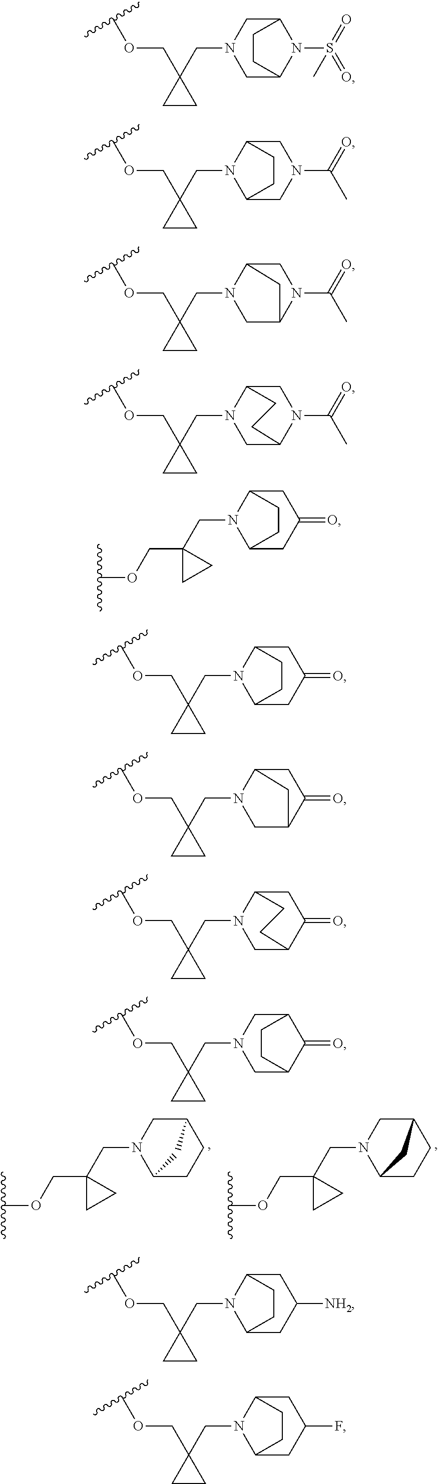

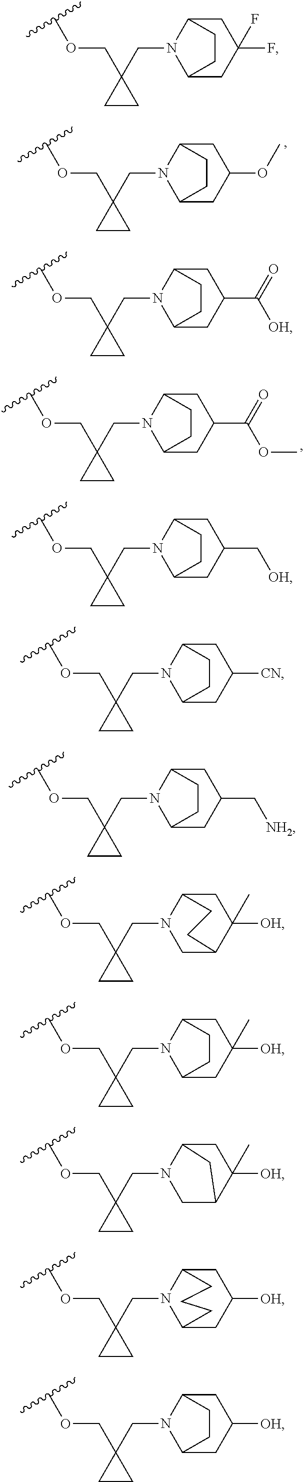

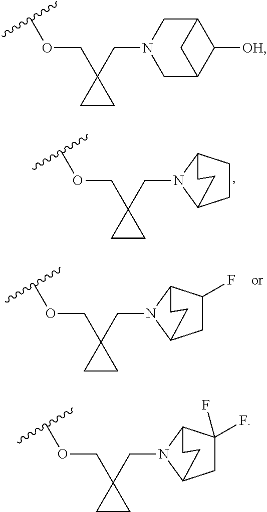 [SMILES: *OCC1(CN2CC3CC(C2)C3O)CC1 ; *OCC1(CN2C3CCC2CC3)CC1 ; *OCC1(CN2C3CCC2C(F)C3)CC1 ; *OCC1(CN2C3CCC2C(F)(F)C3)CC1]

[25]. The compound according to any one of [1] to [2], wherein, the compound of formula (I) is selected from formula (I-B):

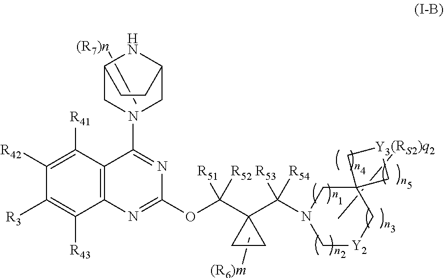

wherein,
Y₂ is selected from —O—, —CH₂—;
Y₃ is selected from —O—, —CH₂—, —NH—, —CHF—, —CF₂—, —S(═O)—, —S(═O)₂—, —C(═O)NH—, or —NHC(═O)—;
R_{S2} at each occurrence is independently selected from —F, —Cl, —Br, —C₁₋₃alkyl, —C₁₋₃haloalkyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —NHC(═O)(C₁₋₃alkyl), —S(═O)NH₂, —S(═O)NH(C₁₋₃alkyl), —NHS(═O)(C₁₋₃alkyl), —S(═O)₂NH₂, —S(═O)₂NH(C₁₋₃alkyl), —NHS(═O)₂(C₁₋₃alkyl), 3-6 membered cycloalkyl, 3-6 membered heterocyclyl, 6-10 membered aryl or 5-10 membered heteroaryl; said —C₁₋₃alkyl, —C₁₋₃haloalkyl, —NH₂, —OH, —SH, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —CH₃, —CH₂CH₃, —CH₂CH₂CH₃, —CH(CH₃)₂, —CH₂F, —CHF₂, —CF₃, oxo, —CN, —COOH, —NH₂, —NH(CH₃), —NH—CH(CH₃)₂, —N(CH₃)₂, —OH, —O(CH₃), —O—CH₂CH₃, —O—CH₂CH₂CH₃, —O—CH(CH₃)₂, —SH, —S(CH₃), —C(═O)—CH₃, —C(═O)—CH₂CH₃, —C(═O)—CF₃, or 3 membered cycloalkyl; preferably, R_{S2} at each occurrence is independently selected from —F, —Cl, —Br, —CH₃, —CH₂CH₃, —CH₂CH₂CH₃, —CH(CH₃)₂, —CH₂F, —CHF₂, —CF₃, —CH₂CH₂F, —CH₂CHF₂, —CH₂CF₃, —CHFCH₃, —CF₂CH₃, —CN, oxo, —NH₂, —NH(CH₃), —N(CH₃)₂, —OH, —O—CH₃, —SH, —S—CH₃, —S(O)—CH₃, —S(O)₂—CH₃, —CHO, —C(O)—CH₃, —COOH, —COOCH₃, —C(═O)NH₂, —C(═O)NH(CH₃), —NH(CHO), —NH—C(═O)CH₃, —S(═O)NH₂, —S(═O)NH(CH₃), —NH—S(═O)CH₃, —S(═O)₂NH₂, —S(═O)₂NH(CH₃), —NH—S(═O)₂CH₃, —CH₂OH, —CH₂O(CH₃), —CH₂NH₂ or —CH₂NH(CH₃); more preferably, R_{S2} at each occurrence is independently selected from —F, —CH₃, —CN, oxo, —NH₂, —OH, —O—CH₃, —COOH, —C(O)—CH₃, —S(O)₂—CH₃, —CH₂NH₂ or —CH₂OH;
q₂ is selected from 0, 1, 2, 3, 4, 5 or 6; preferably, q₂ is selected from 0, 1, 2, or 3.

[26]. The compound according to any one of [1], [2] and [25], wherein, the compound of formula (I-B) is selected from any one of formula (I-B1) to (I-B9):

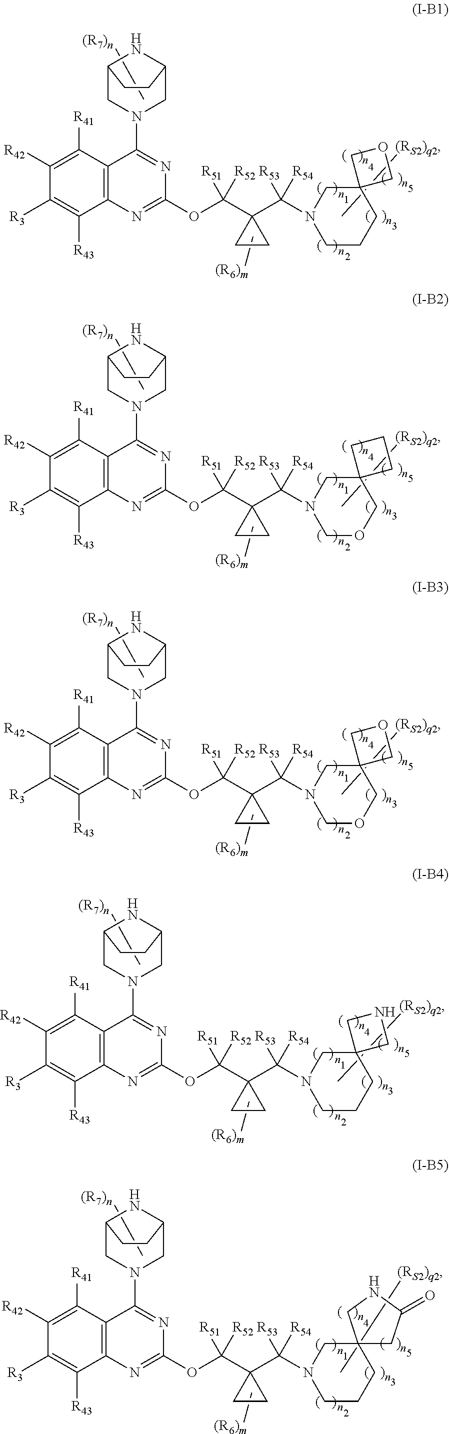

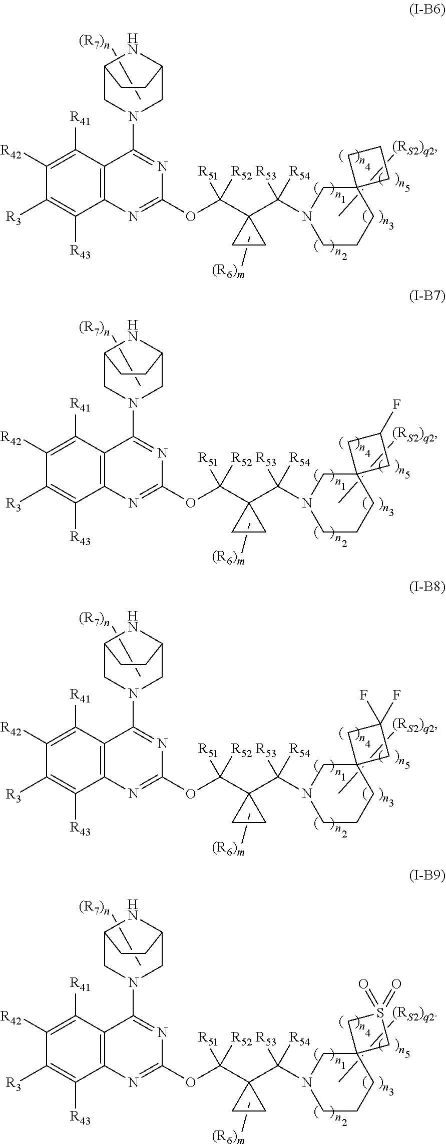

[27]. The compound according to any one of [1], [2] and [25] to [26], wherein, the compound of formula (I) is selected from formula (I-B1):

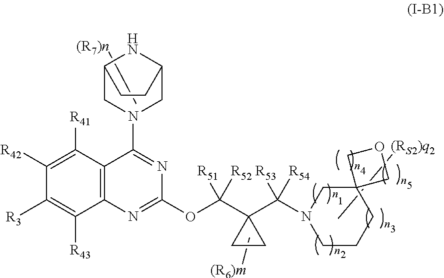

wherein, n₁ is selected from 1 or 2; n₂ is selected from 0 or 1; n₃ is selected from 0; n₄ is selected from 0, 1 or 2; n₅ is selected from 1, 2, 3, or 4.

[28]. The compound according to any one of [27], wherein, the moiety of

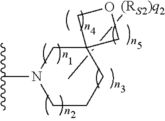

in the formula (I-B1) is selected from

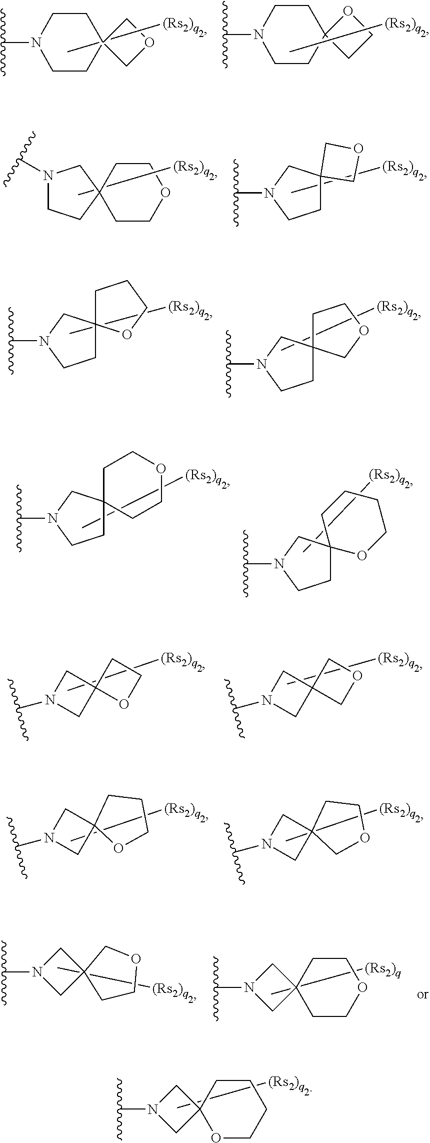

[29]. The compound according to any one of [27] to [28], wherein, the moiety of

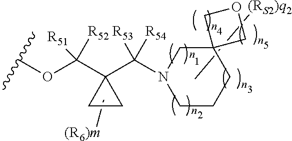

in formula (I-B1) is selected from

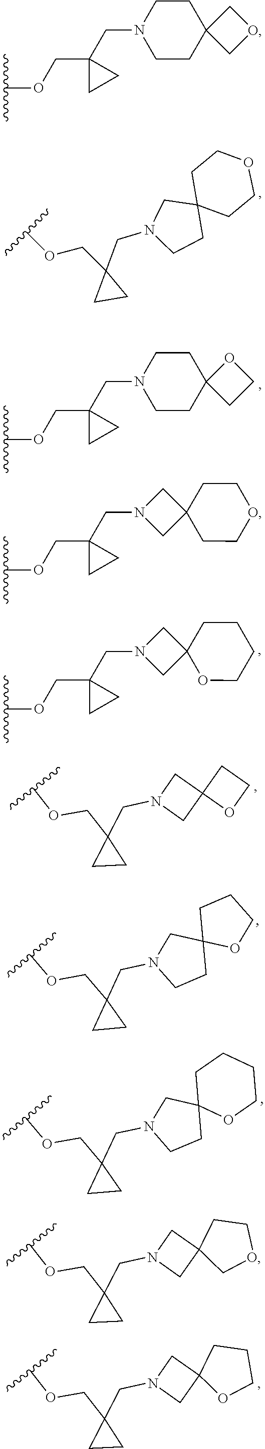

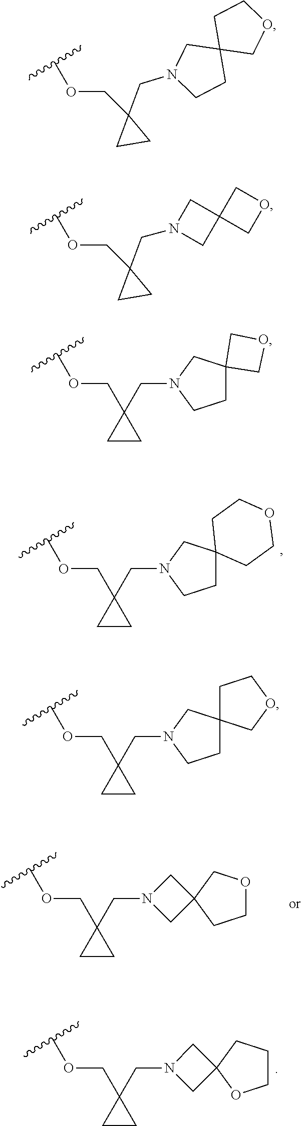

[30]. The compound according to any one of [1], [2] and [25] to [26], wherein, the compound of formula (I) is selected from formula (I-B2):

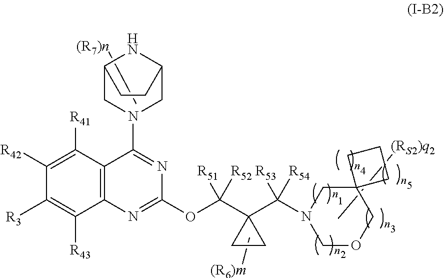

Wherein, n₁ is selected from 0 or 1; n₂ is selected from 2; n₃ is selected from 0 or 1; n₄ is selected from 0 or 1; n₅ is selected from 1.

[31]. The compound according to [30], wherein, the moiety of

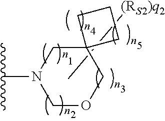

in the formula (I-B2) is selected from

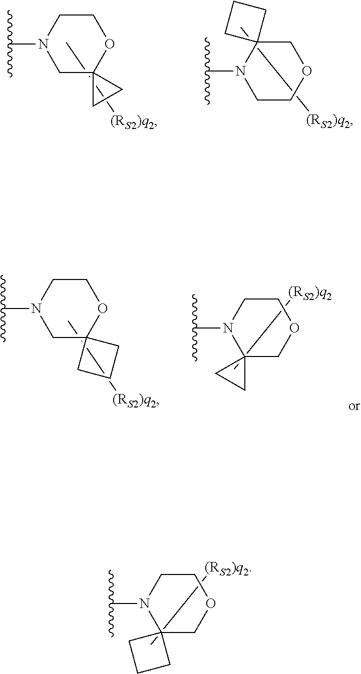

[32]. The compound according to any one of [30] to [31], wherein, the moiety of

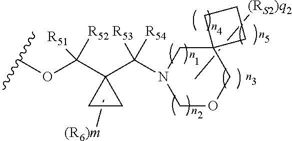

in the formula (I-B2) is selected from

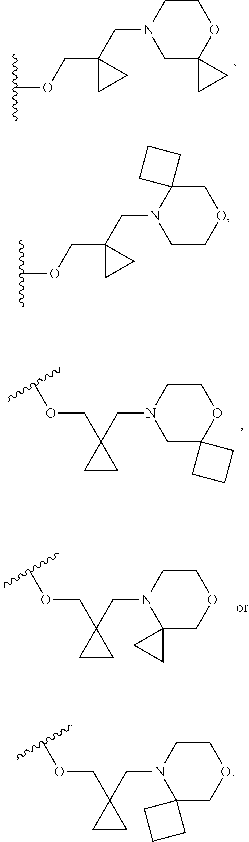 [SMILES: *OCC1(CN2CCOC3(CC3)C2)CC1 ; *OCC1(CN2CCOCC23CCC3)CC1 ; *OCC1(CN2CCOC3(CCC3)C2)CC1 ; *OCC1(CN2CCOCC23CC3)CC1 ; *OCC1(CN2CCOCC23CCC3)CC1]

[33]. The compound according to any one of [1], [2] and [25] to [26], wherein, the compound of formula (I) is selected from formula (I-B3):

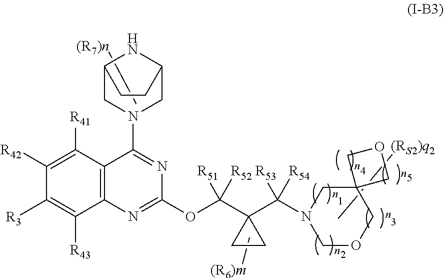

wherein, n₁ is selected from 0 or 1; n₂ is selected from 2; n₃ is selected from 0 or 1; n₄ is selected from 1; n₅ is selected from 1.

[34]. The compound according to [33], wherein, the moiety of

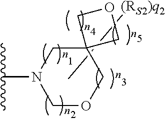

in formula (I-B3) is selected from

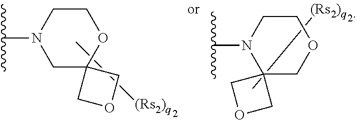

[35]. The compound according to any one of [33] to [34], wherein, the moiety of

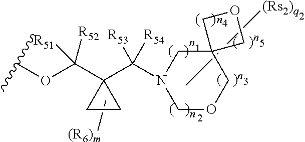

in the formula (I-B3) is selected from

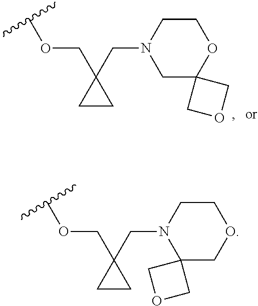 [SMILES: *OCC1(CN2CCOC3(COC3)C2)CC1 ; *OCC1(CN2CCOCC23COC3)CC1]

[36]. The compound according to any one of [1], [2] and [25] to [26], wherein, the compound of formula (I) is selected from formula (I-B4):

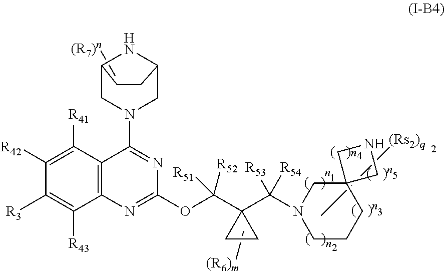

wherein, n₁ is selected from 1 or 2; n₂ is selected from 0 or 1; n₃ is selected from 0; n₄ is selected from 0, 1, 2, 3 or 4; n₅ is selected from 0, 1 or 2;
R_{S2} at each occurrence are independently selected from —C₁₋₃alkyl, —S(═O)₂C₁₋₃alkyl, or —C(═O)C₁₋₃alkyl; preferably, R_{S2} at each occurrence is independently selected from —CH₃, —CH₂CH₃, —CH₂CH₂CH₃, —CH(CH₃)₂, —S(═O)₂CH₃ or —C(═O)CH₃.
q₂ is selected from 0, 1, or 2.

[37]. The compound according to any one of [36], wherein, the moiety of

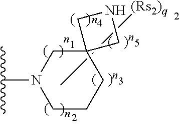

in the formula (I-B4) is selected from

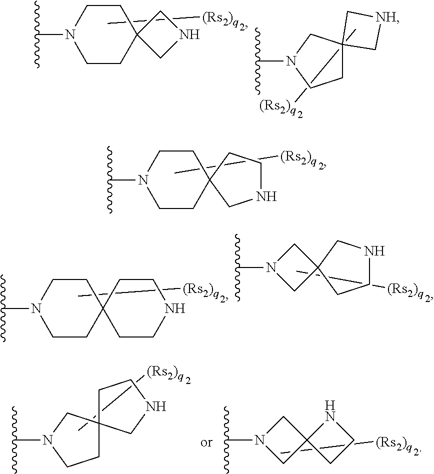

[38]. The compound according to any one of [36] to [37], wherein, the moiety of

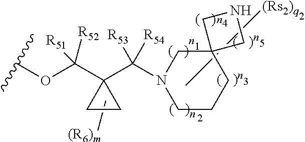

in the formula (I-B4) is selected from

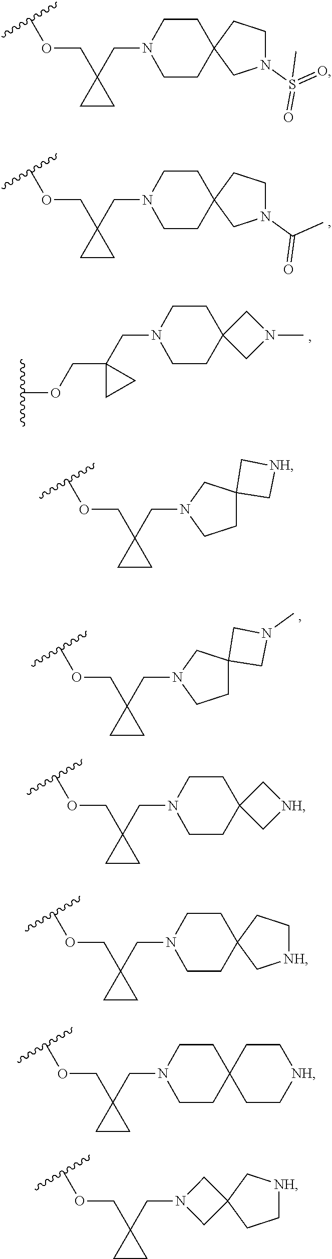

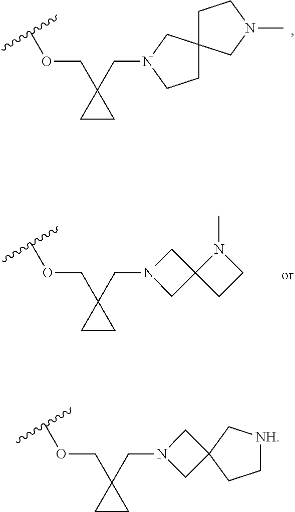 [SMILES: *OCC1(CN2CCC3(CCN(C)C3)C2)CC1 ; *OCC1(CN2CC3(CCN3C)C2)CC1 ; *OCC1(CN2CC3(CCNC3)C2)CC1]

[39]. The compound according to any one of [1], [2] and [25] to [26], wherein, the compound of formula (I) is selected from formula (I-B5):

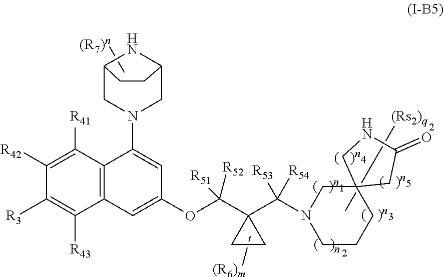

Wherein, n₁ is selected from 1 or 2; n₂ is selected from 1; n₃ is selected from 0; n₄ is selected from 0, 1, 2, or 3; n₅ is selected from 0 or 2;
R_{S2} at each occurrence are independently selected from —C₁₋₃alkyl; preferably, R_{S2} at each occurrence is independently selected from —CH₃, —CH₂CH₃, —CH₂CH₂CH₃ or —CH(CH₃)₂;
q₂ is selected from 0, or 1.

[40]. The compound according to [39], wherein, the moiety of

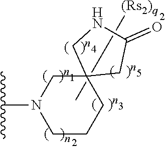

in the formula (I-B5) is selected from

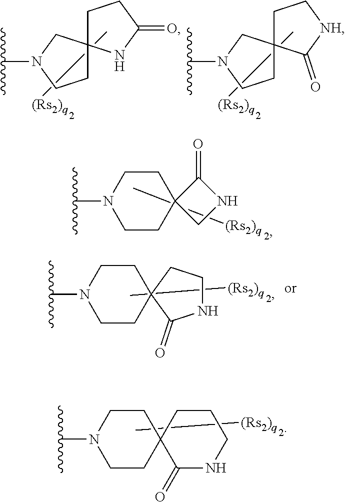

[41]. The compound according to any one of [39] to [40], wherein, the moiety of

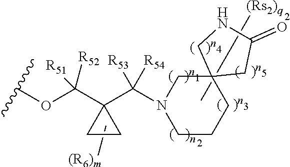

in the formula (I-B5) is selected from

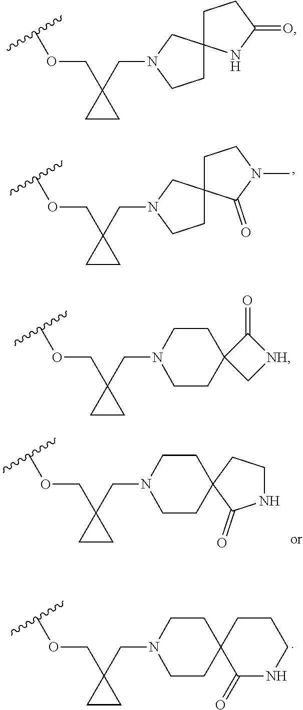 [SMILES: *OCC1(CN2CCC3(CCC(=O)N3)C2)CC1 ; *OCC1(CN2CCC3(CCN(C)C3=O)C2)CC1 ; *OCC1(CN2CCC3(CC2)CNC3=O)CC1 ; *OCC1(CN2CCC3(CCNC3=O)CC2)CC1 ; *OCC1(CN2CCC3(CCCNC3=O)CC2)CC1]

[42]. The compound according to any one of [1], [2] and [25] to [26], wherein, the compound of formula (I) is selected from formula (I-B6):

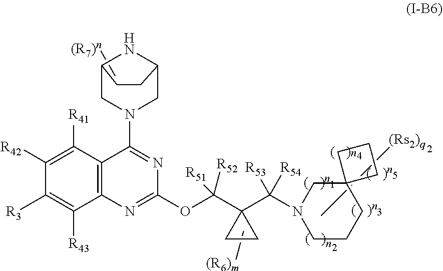

wherein,
n₁ is selected from 1 or 2; n₂ is selected from 0 or 1; n₃ is selected from 0 or 1; n₄ is selected from 1 or 2; n₅ is selected from 1 or 2;
R_{S2} at each occurrence is independently selected from —F, —Cl, —Br, —C₁₋₃alkyl, —NH₂, —OH, or —O(C₁₋₃alkyl); preferably, R_{S2} at each occurrence is independently selected from —F, —Cl, —CH₃, —CH₂CH₃, —CH₂CH₂CH₃, —CH(CH₃)₂, —NH₂, —OH, or —OCH₃;
q₂ is selected from 0, 1, or 2.

[43]. The compound according to [42], wherein, the moiety of

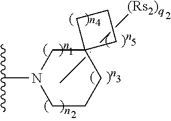

in the formula (I-B6) is selected from

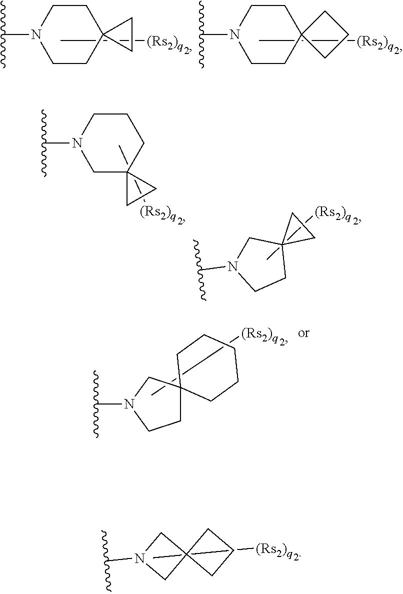

[44]. The compound according to any one of [42] to [43], wherein, the moiety of

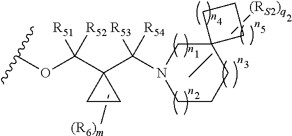

in the formula (I-B6) is selected from

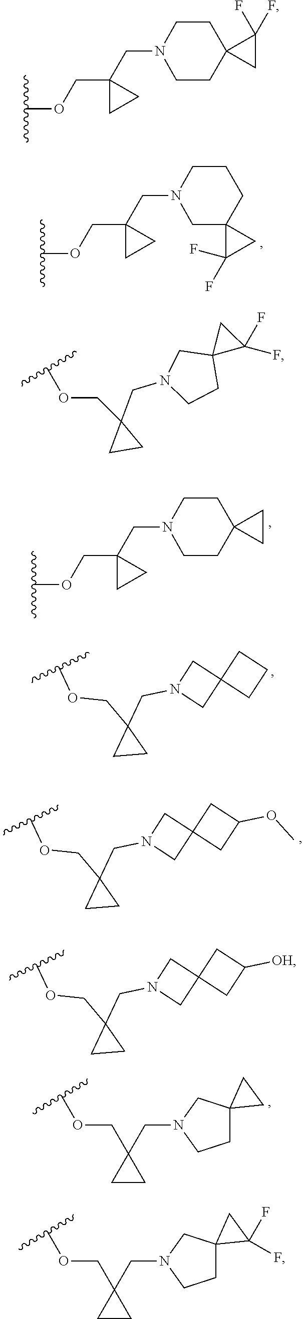

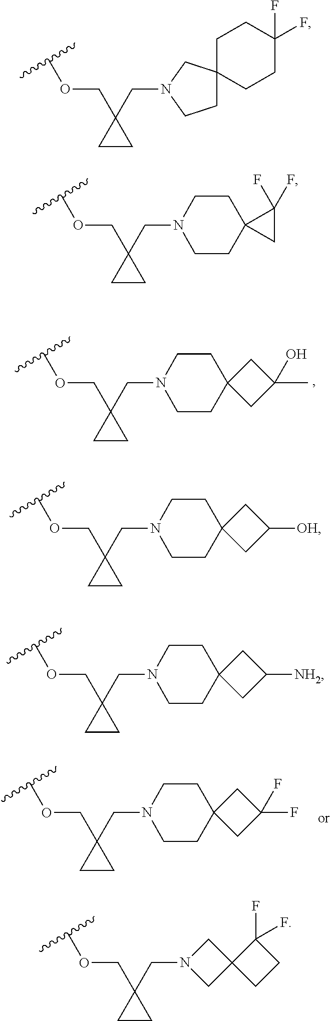

[45]. The compound according to any one of [1], [2] and [25] to [26], wherein, the compound of formula (I) is selected from formula (I-B8):

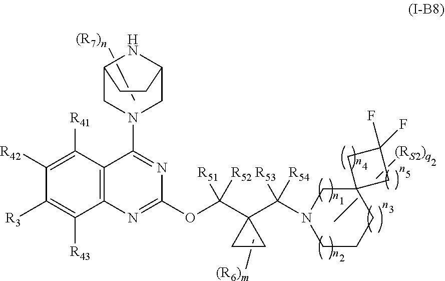

wherein, n₁ is selected from 1 or 2; n₂ is selected from 0 or 1; n₃ is selected from 0 or 1; n₄ is selected from 0, 1 or 2; n₅ is selected from 1 or 2.

[46]. The compound according to [45], wherein, the moiety of

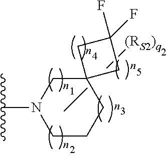

in the formula (I-B8) is selected from

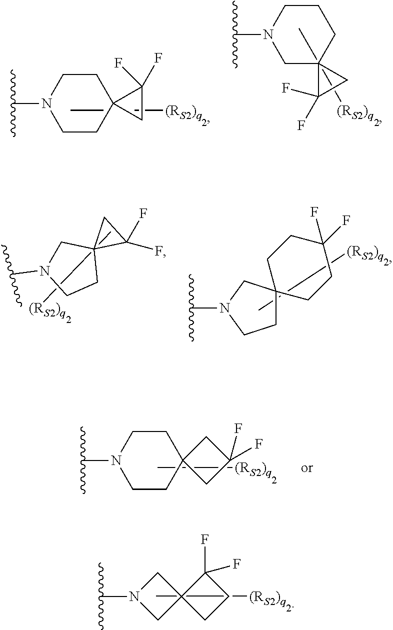

[47]. The compound according to any one of [45] to [46], wherein, the moiety of

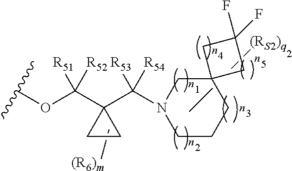

in the formula (I-B8) is selected from

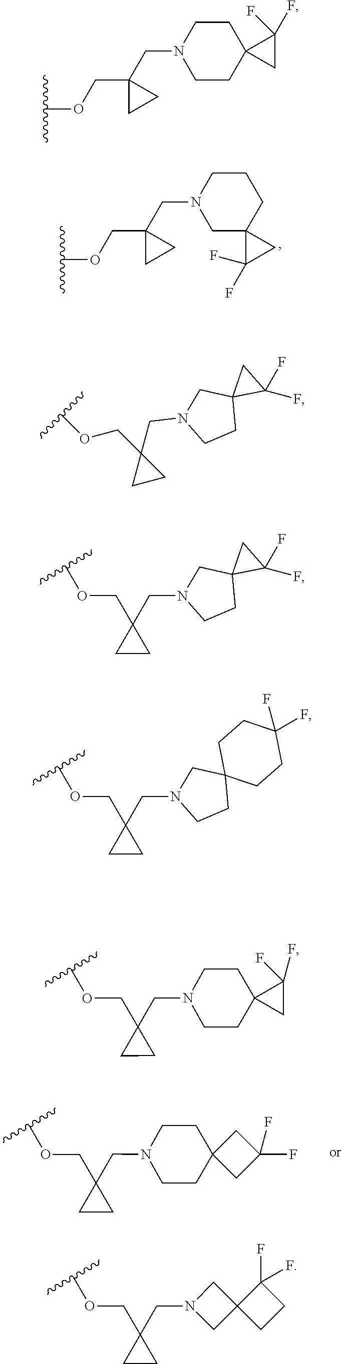

[48]. The compound according to any one of [1], [2] and [25] to [26], wherein, the compound of formula (I) is selected from formula (I-B9):

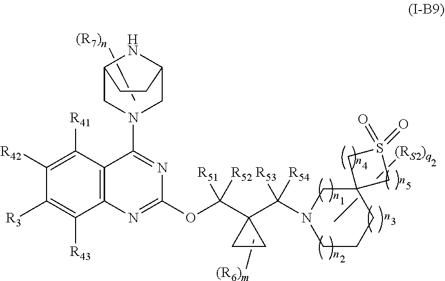

Wherein, n₁ is selected from 1; n₂ is selected from 0; n₃ is selected from 0; n₄ is selected from 1; n₅ is selected from 1 or 2.

[49]. The compound according to [48], wherein, the moiety of

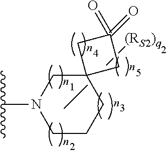

in the formula (I-B9) is selected from

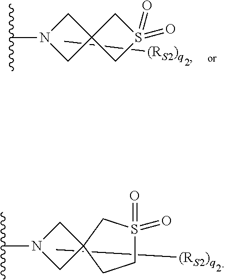

[50]. The compound according to any one of [48] to [49], wherein, the moiety of

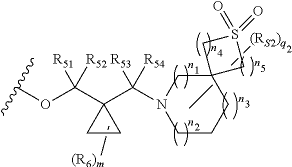

in the formula (I-B9) is selected from

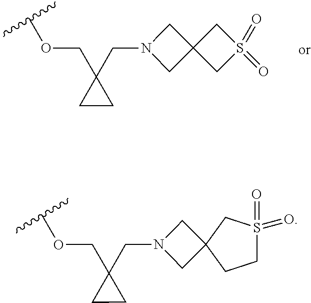 [SMILES: *OCC1(CN2CC3(C2)CS(=O)(=O)C3)CC1 ; *OCC1(CN2CC3(CCS(=O)(=O)C3)C2)CC1]

[51]. The compound according to any one of [1], [2] and [25] to [50], wherein, the moiety of

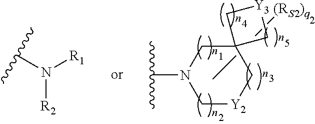

is selected from

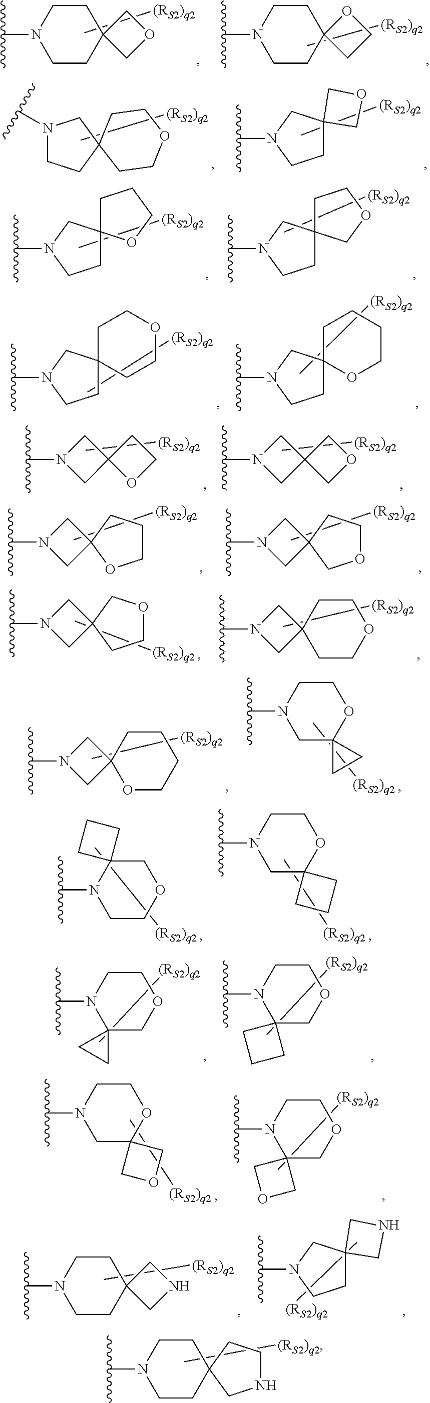

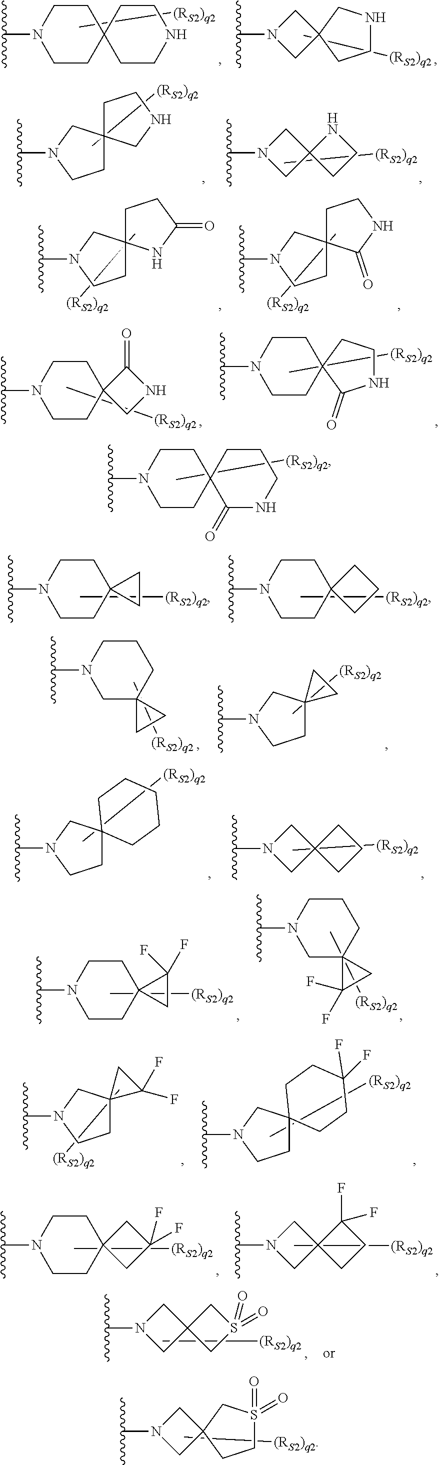

[52]. The compound according to any one of [1], [2] and [25] to [51], wherein, the moiety of

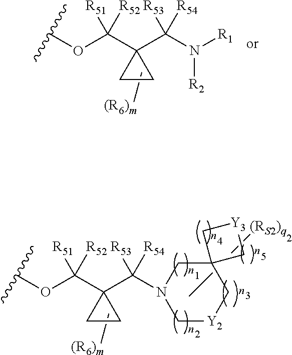

is selected from

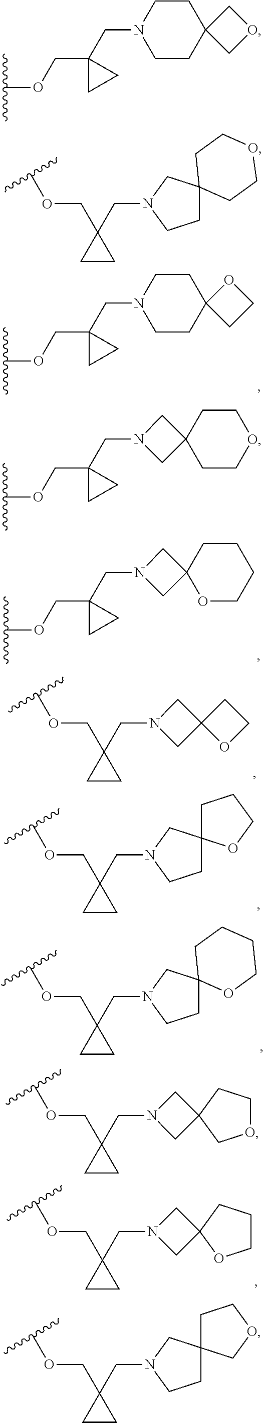

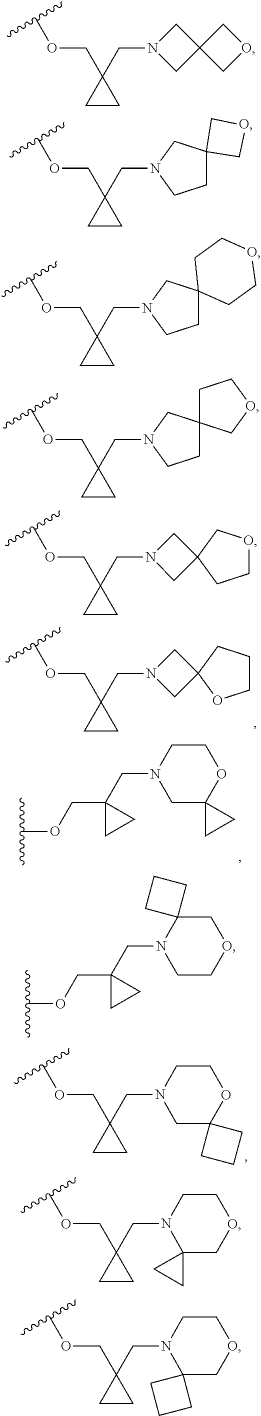

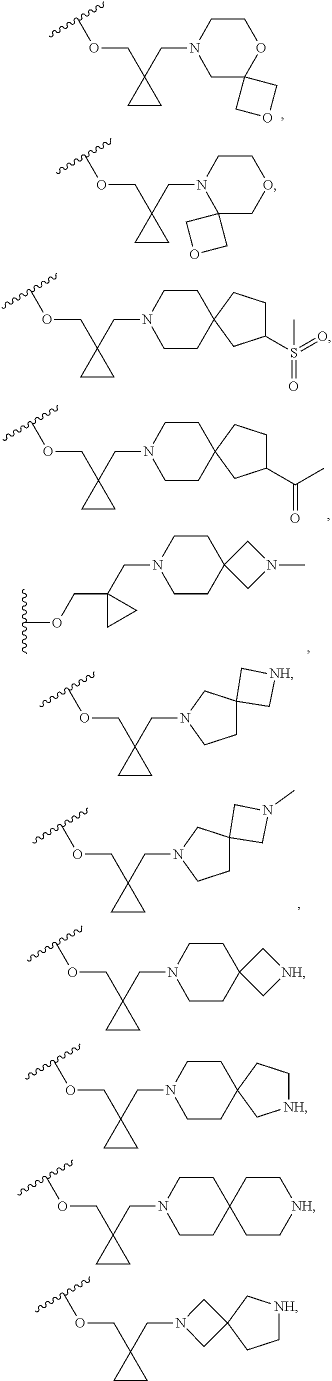

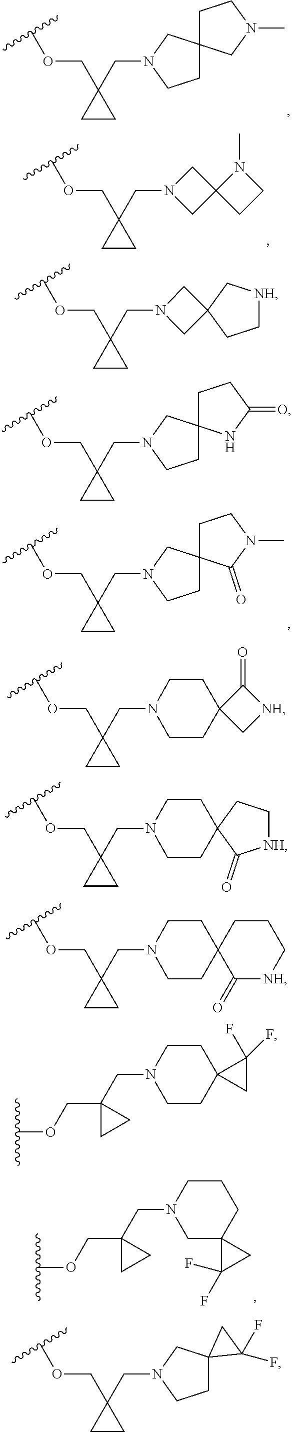

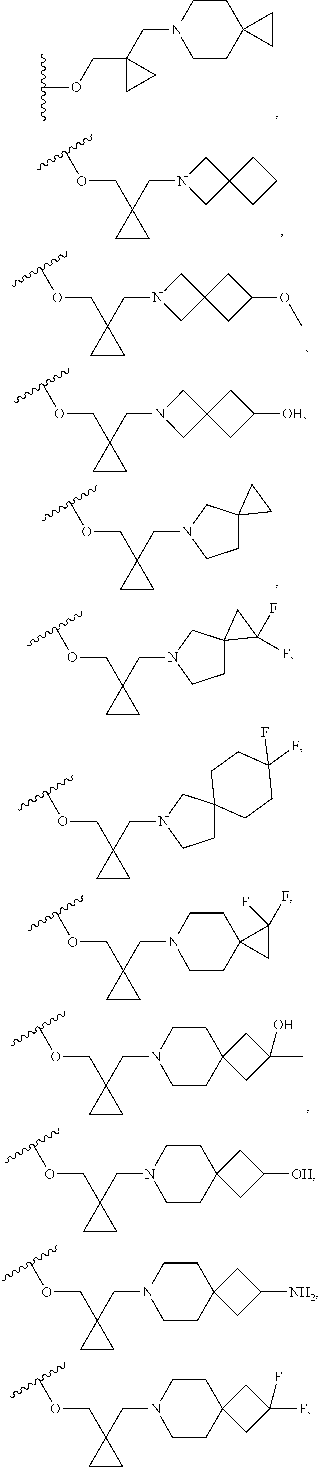

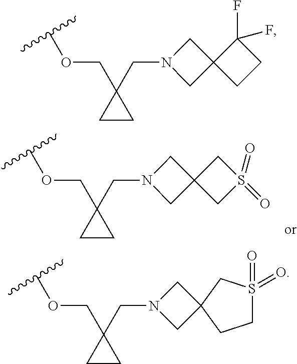 [SMILES: *OCC1(CN2CC3(CCC3(F)F)C2)CC1 ; *OCC1(CN2CC3(C2)CS(=O)(=O)C3)CC1 ; *OCC1(CN2CC3(CCS(=O)(=O)C3)C2)CC1]

[53]. The compound according to any one of [1] to [2], wherein, the compound of formula (I) is selected from formula (I-C):

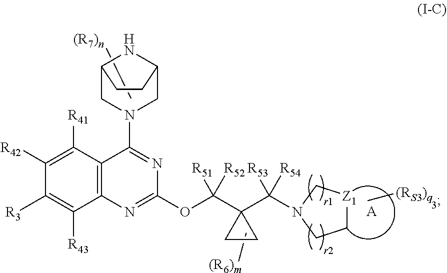

When Z₁ is selected from CH, the compound of formula (I-C) is selected from formula (I-C1) or formula (I-C2):

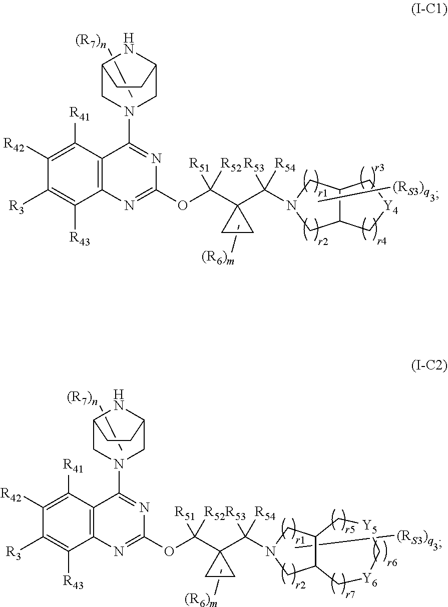

r₁ is selected from 0, 1, or 2; r₂ is selected from 0, 1, or 2; r₃ is selected from 0, 1, or 2; r₄ is selected from 0, 1, or 2; r₅ is selected from 0, 1, or 2; r₆ is selected from 0, 1, or 2; r₇ is selected from 0, 1, or 2;
Y₄ is selected from —O—, —S—, —S(═O)—, —S(═O)₂—, —C(═O)—, —NH—, —CH₂—, —CHF—, or —CF₂—;
Y₅ is selected from —O—, —S—, —S(═O)—, —S(═O)₂—, —C(═O)—, —NH—, —CH₂—, —CHF—, or —CF₂—;
Y₆ is selected from —O—, —S—, —S(═O)—, —S(═O)₂—, —C(═O)—, —NH—, —CH₂—, —CHF—, or —CF₂—;

When Z₁ is selected from C or N, the compound of formula (I-C) is selected from formula (I-C3) or formula (I-C4):

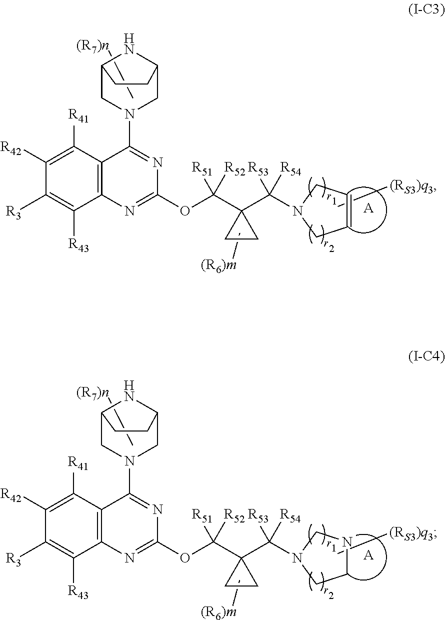

said ring A in the compound of formula (I-C3) is selected from a phenyl ring; 5 membered heteroaryl ring including 1 or 2 ring members selected from N, O or S; or 6 membered heteroaryl ring including 1, 2 or 3 ring members selected from N, O or S; preferably, said ring A in the compound of formula (I-C3) is selected from a phenyl ring; 5 membered heteroaryl ring including 1 ring members selected from S; or 6 membered heteroaryl ring including 1 ring members selected from N;
said ring A in the compound of formula (I-C4) is selected from a 5 membered heteroaryl ring including 1 ring member selected from N and further containing 1 or 2 ring members selected from N, O or S; or 6 membered heteroaryl ring including 1 ring member selected from N and further containing 1, 2 or 3 ring members selected from N, O or S; preferably, ring A in the compound of formula (I-C4) is selected from a 5 membered heteroaryl ring including 1 ring member selected from N and further containing 1 ring member selected from N;
R_{S3} at each occurrence is independently selected from —F, —Cl, —Br, —C₁₋₃alkyl, —C₁₋₃haloalkyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —NHC(═O)(C₁₋₃alkyl), —S(═O)NH₂, —S(═O)NH(C₁₋₃alkyl), —NHS(═O)(C₁₋₃alkyl), —S(═O)₂NH₂, —S(═O)₂NH(C₁₋₃alkyl), —NHS(═O)₂(C₁₋₃alkyl), 3-6 membered cycloalkyl, 3-6 membered heterocyclyl, 6-10 membered aryl or 5-10 membered heteroaryl; said —C₁₋₃alkyl, —C₁₋₃haloalkyl, —NH₂, —OH, —SH, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —CH₃, —CH₂CH₃, —CH₂CH₂CH₃, —CH(CH₃)₂, —CH₂F, —CHF₂, —CF₃, oxo, —CN, —COOH, —NH₂, —NH(CH₃), —NH—CH(CH₃)₂, —N(CH₃)₂, —OH, —O(CH₃), —O—CH₂CH₃, —O—CH₂CH₂CH₃, —O—CH(CH₃)₂, —SH, —S(CH₃), —C(═O)CH₃, —C(═O)CH₂CH₃, —C(═O)—CF₃, or 3 membered cycloalkyl; preferably, R_{S3} at each occurrence is independently selected from —F, —Cl, —Br, —CH₃, —CH₂CH₃, —CH₂CH₂CH₃, —CH(CH₃)₂, —CH₂F, —CHF₂, —CF₃, —CH₂CH₂F, —CH₂CHF₂, —CH₂CF₃, —CHFCH₃, —CF₂CH₃, —CN, oxo, —NH₂, —NH(CH₃), —N(CH₃)₂, —OH, —OCH₃, —SH, —SCH₃, —S(═O)CH₃, —S(═O)₂—CH₃, —CHO, —C(═O)CH₃, —COOH, —COOCH₃, —C(═O)NH₂, —C(═O)NH(CH₃), —NH(CHO), —NH—C(═O)CH₃, —S(═O)NH₂, —S(═O)NH(CH₃), —NHS(═O)CH₃, —S(═O)₂NH₂, —S(═O)₂NH(CH₃), —NHS(═O)₂CH₃, —CH₂OH, —CH₂O(CH₃), —CH₂NH₂ or —CH₂NH(CH₃); more preferably, R_{S3} at each occurrence is independently selected from —F, —CH₃, —CN, oxo, —NH₂, —OH, —OCH₃, —COOH, —C(═O)CH₃, —S(═O)₂CH₃, —CH₂NH₂ or —CH₂OH;
q₃ is selected from 0, 1, 2, 3, 4, 5 or 6; preferably, q₃ is selected from 0, 1, 2, or 3.

[54]. The compound according to any one of [1], [2], and [53], wherein, the compound of formula (I-C1) is selected from any one of formula (I-C1-1) to formula (I-C1-5):

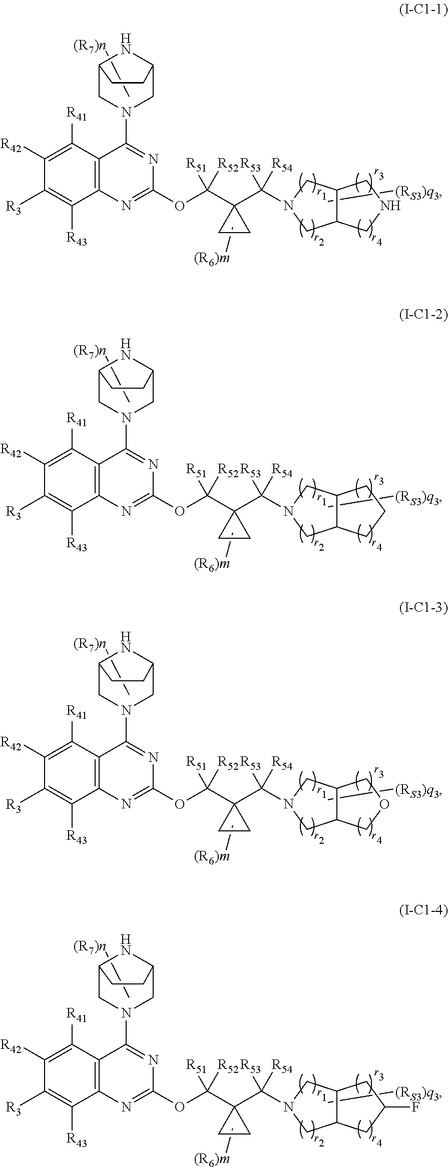

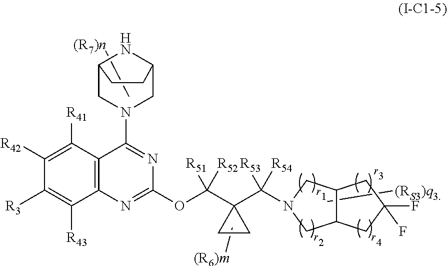

[55]. The compound according to any one of [1], [2], and [53] to [54], wherein, the compound of formula (I-C1) is selected from formula (I-C1-1):

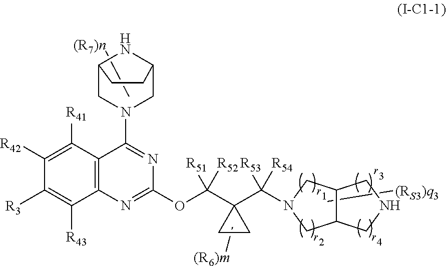

wherein,
r₁ is selected from 1; r₂ is selected from 0, 1 or 2; r₃ is selected from 0, or 1; r₄ is selected from 0 or 1;
R_{S3} at each occurrence is independently selected from —CH₃, —CH₂CH₃, —CH₂CH₂CH₃, or —CH(CH₃)₂; preferably, R_{S3} at each occurrence is independently selected from —CH₃;
q₃ is selected from 0 or 1.

[56]. The compound according to [55], wherein, the moiety of

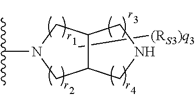

in the formula (I-C1-1) is selected from

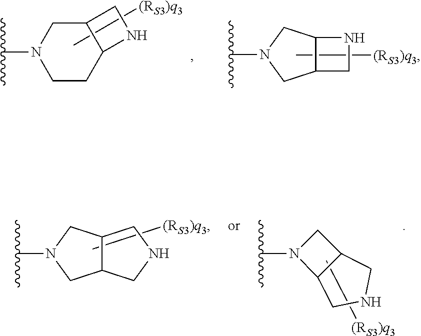

[57]. The compound according to any one of [55] to [56], wherein, the moiety of

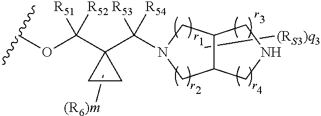

in formula (I-C1-1) is selected from

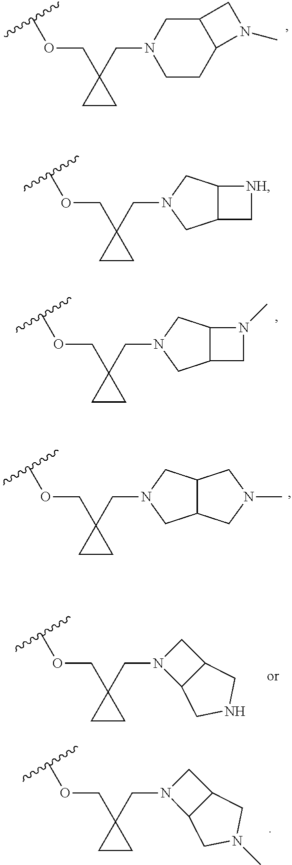 [SMILES: *OCC1(CN2CCC3C(C2)CN3C)CC1 ; *OCC1(CN2CC3CNC3C2)CC1 ; *OCC1(CN2CC3CN(C)C3C2)CC1 ; *OCC1(CN2CC3CN(C)CC3C2)CC1 ; *OCC1(CN2CC3CNCC32)CC1 ; *OCC1(CN2CC3CN(C)CC32)CC1]

[58]. The compound according to any one of [1], [2], and [53] to [54], wherein, the compound of formula (I-C1) is selected from formula (I-C1-2):

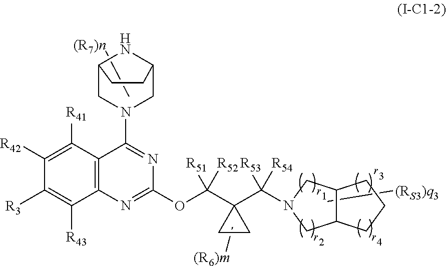

wherein, r₁ is selected from 1; r₂ is selected from 1; r₃ is selected from 1; r₄ is selected from 0 or 1;
R_{S3} at each occurrence is independently selected from —F, —Cl, —OH, or —NH₂;
q₃ is selected from 0, 1 or 2.

[59]. The compound according to [58], wherein, the moiety of

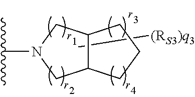

in the formula (I-C1-2) is selected from

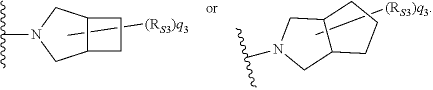

[60]. The compound according to any one of [58] to [59], wherein, the moiety of

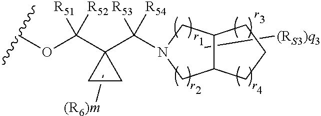

in formula (I-C1-2) is selected from

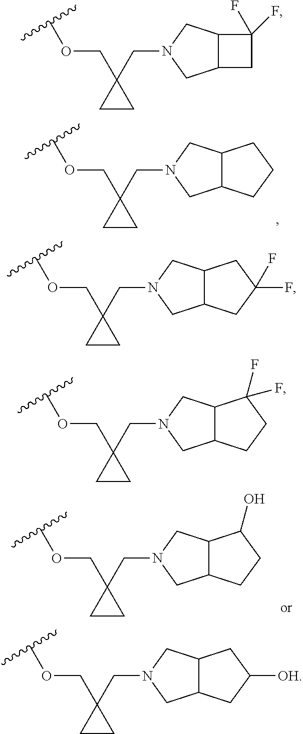 [SMILES: *OCC1(CN2CC3CC(F)(F)C3C2)CC1 ; *OCC1(CN2CC3CCCC3C2)CC1 ; *OCC1(CN2CC3CC(F)(F)CC3C2)CC1 ; *OCC1(CN2CC3CCC(F)(F)C3C2)CC1 ; *OCC1(CN2CC3CCC(O)C3C2)CC1 ; *OCC1(CN2CC3CC(O)CC3C2)CC1]

[61]. The compound according to any one of [1], [2], and [53] to [54], wherein, the compound of formula (I-C1) is selected from formula (I-C1-3):

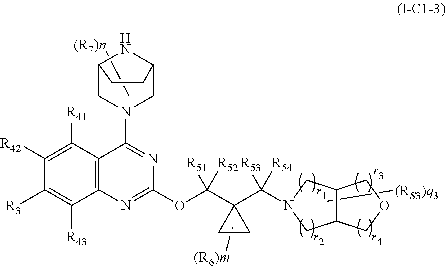

r₁ is selected from 1; r₂ is selected from 1; r₃ is selected from 1; r₄ is selected from 1.

[62]. The compound according to [61], the moiety of

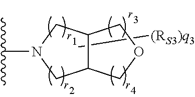

in the formula (I-C1-3) is selected from

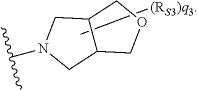

[63]. The compound according to any one of [61] to [62], wherein, the moiety of

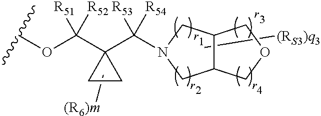

in formula (I-C1-3) is selected from

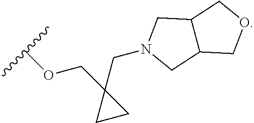 [SMILES: *OCC1(CN2CC3COCC3C2)CC1]

[64]. The compound according to any one of [1], [2], and [53] to [54], wherein, the compound of formula (I-C1) is selected from formula (I-C1-5):

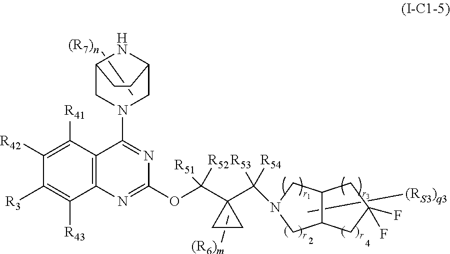

Wherein, r₁ is selected from 1; r₂ is selected from 1; r₃ is selected from 0, or 1; r₄ is selected from 1 or 2.

[65]. The compound according to [64], wherein, the moiety of

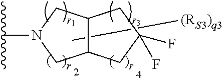

in formula (I-C1-5) is selected from

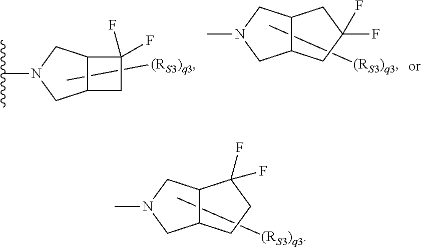

[66]. The compound according to any one of [64] to [65], wherein, the moiety of

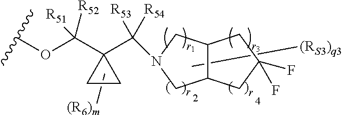

in formula (I-C1-5) is selected from

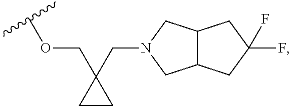 [SMILES: *OCC1(CN2CC3CC(F)(F)CC3C2)CC1]

[67]. The compound according to any one of [1], [2], and [53], wherein, the compound of formula (I-C2) is selected from formula (I-C2-1) or formula (I-C2-2):

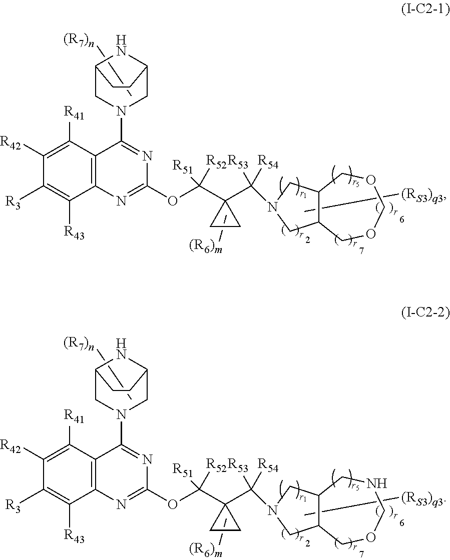

[68]. The compound according to any one of [1], [2], [53], and [67], wherein, the compound of formula (I-C2) is selected from formula (I-C2-1):

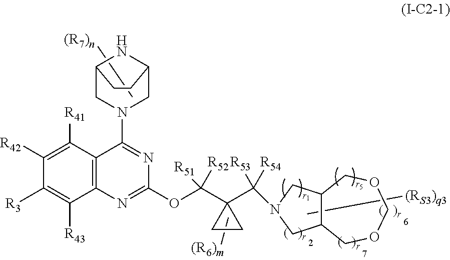

Wherein, r₁ is selected from 1; r₂ is selected from 1; r₅ is selected from 0; r₇ is selected from 0; r₆ is selected from 2.

[69]. The compound according to [68], wherein, the moiety of

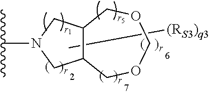

in formula (I-C2-1) is selected from

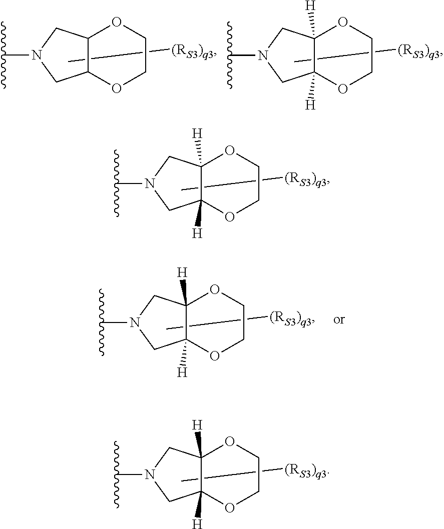

[70]. The compound according to any one of [68] to [69], wherein, the moiety of

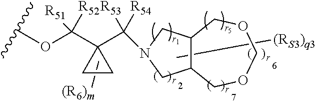

in formula (I-C2-1) is selected from

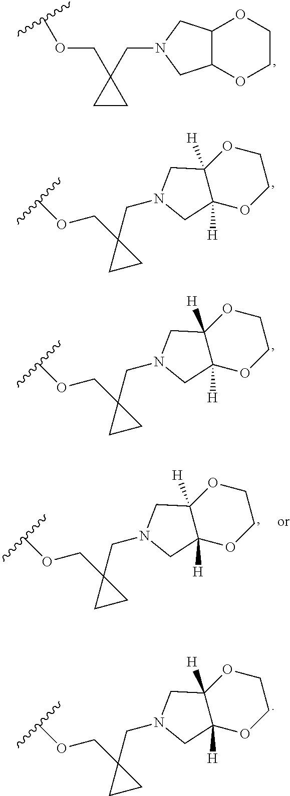 [SMILES: *OCC1(CN2CC3OCCOC3C2)CC1 ; *OCC1(CN2C[C@@H]3OCCO[C@@H]3C2)CC1 ; *OCC1(CN2C[C@@H]3OCCO[C@H]3C2)CC1 ; *OCC1(CN2C[C@H]3OCCO[C@@H]3C2)CC1 ; *OCC1(CN2C[C@@H]3OCCO[C@@H]3C2)CC1]

[71]. The compound according to any one of [1], [2], [53], and [67], wherein, the compound of formula (I-C2) is selected from formula (I-C2-2):

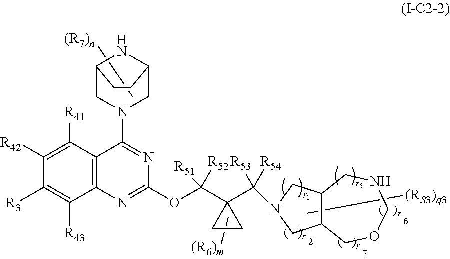

wherein, r₁ is selected from 1; r₂ is selected from 1; r₅ is selected from 0; r₇ is selected from 0; r₆ is selected from 2.

[72]. The compound according to any one of [71], wherein, the moiety of

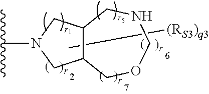

in formula (I-C2-2) is selected from

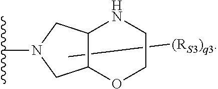

[73]. The compound according to any one of [71] to [72], the moiety of

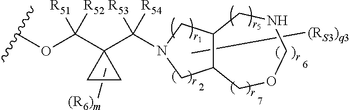

in the formula (I-C2-2) is selected from

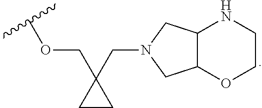 [SMILES: *OCC1(CN2CC3NCCOC3C2)CC1]

[74]. The compound according to any one of [1], [2], [53], and [67], wherein, the compound of formula (I-C3) is selected from formula (I-C3-1), formula (I-C3-2), or formula (I-C3-3):

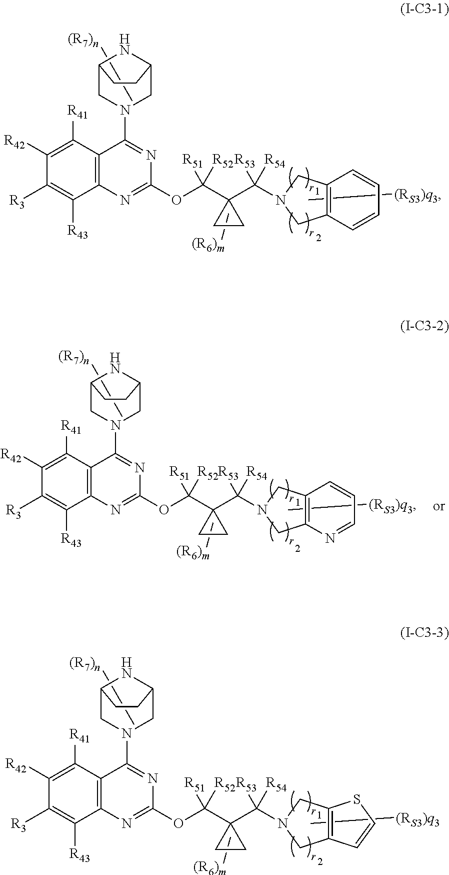

wherein, r₁ is selected from 1 or 2; r₂ is selected from 1.

[75]. The compound according to any one of [74], wherein,
the moiety of

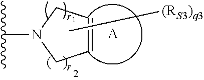

in formula (I-C3) or

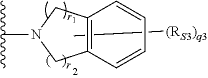

in formula (I-C3-1) is selected from

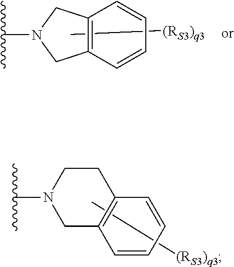

the moiety of

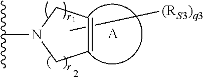

in formula (I-C3) or

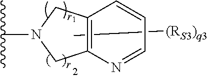

in formula (I-C3-2) is selected from

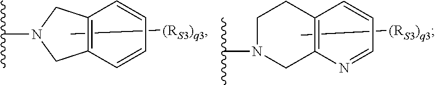

the moiety of

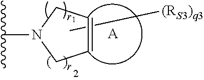

in formula (I-C3) or

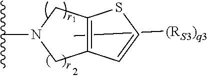

in formula (I-C3-3) is selected from

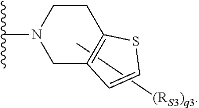

[76]. The compound according to any one of [74] to [75], wherein,
the moiety of

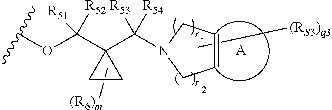

in formula (I-C3) or

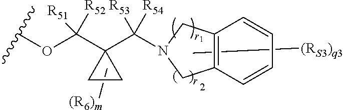

in formula (I-C3-1) is selected from

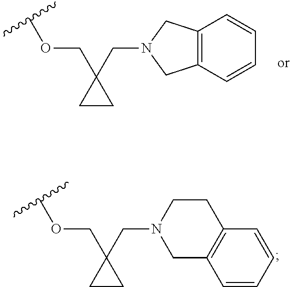 [SMILES: *OCC1(CN2Cc3ccccc3C2)CC1 ; *OCC1(CN2CCc3ccccc3C2)CC1]

the moiety of

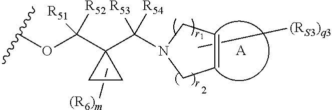

in formula (I-C3) or

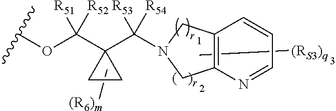

in formula (I-C3-2) is selected from

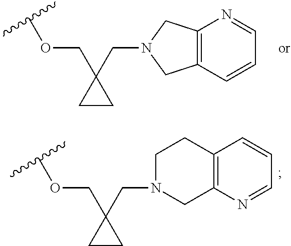 [SMILES: *OCC1(CN2Cc3cccnc3C2)CC1 ; *OCC1(CN2CCc3cccnc3C2)CC1]

the moiety of

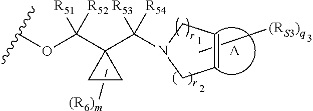

in formula (I-C3) or

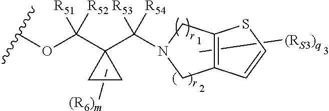

in formula (I-C3-3) is selected from

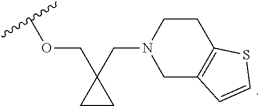 [SMILES: *OCC1(CN2CCc3sccc3C2)CC1]

[77]. The compound according to any one of [1], [2], [53], and [67], wherein, the compound of formula (I-C4) is selected from formula (I-C4-1),

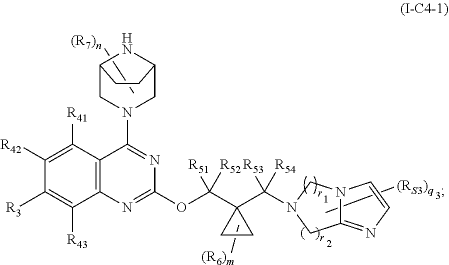

wherein, r₁ is selected from 1 or 2; r₂ is selected from 1.

[78]. The compound according to any one of [77], wherein,
the moiety of

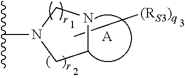

in formula (I-C4) or

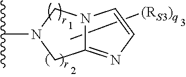

in formula (I-C4-1) is selected from

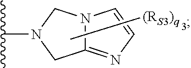

[79]. The compound according to any one of [77] or [78], wherein,
the moiety of

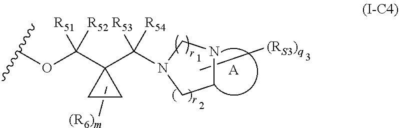

in formula (I-C4) or

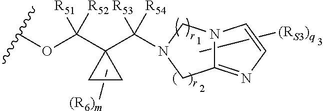

in formula (I-C4-1) is selected from

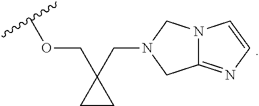 [SMILES: *OCC1(CN2Cc3nccn3C2)CC1]

[80]. The compound according to any one of [1] to [2], and [53] to [79], wherein, the moiety of

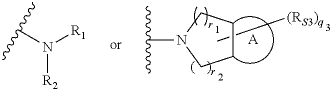

is selected from

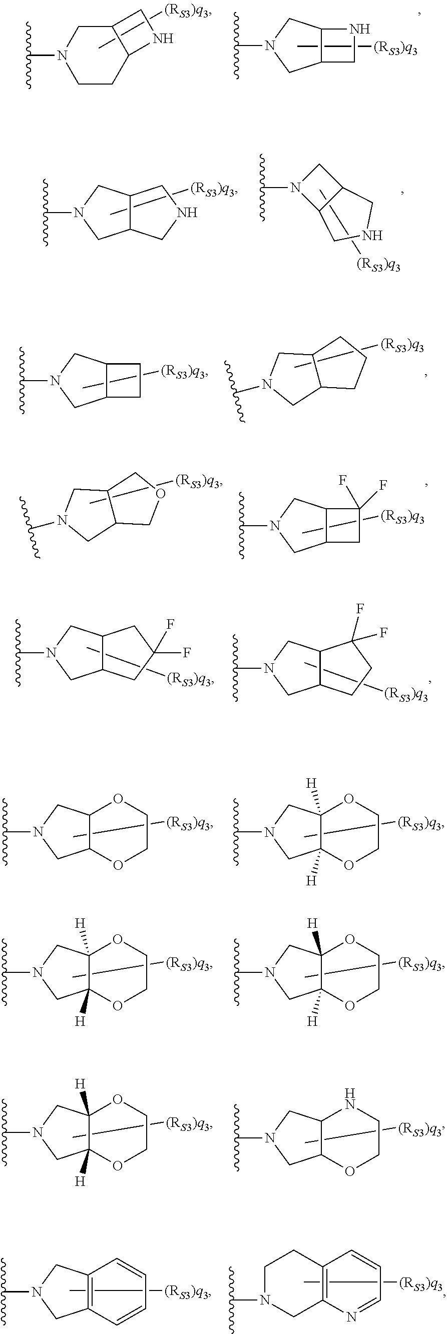

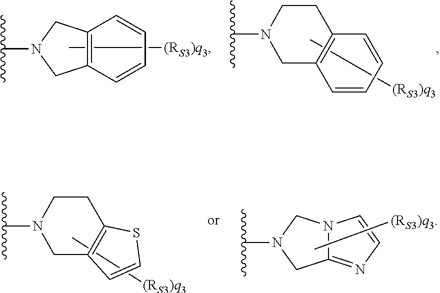

[81]. The compound according to any one of [1] to [2], and [53] to [80], wherein, the moiety of

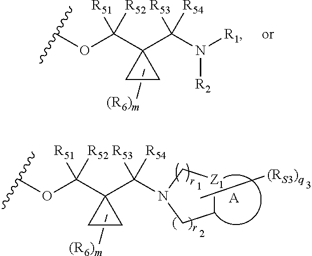

is selected from

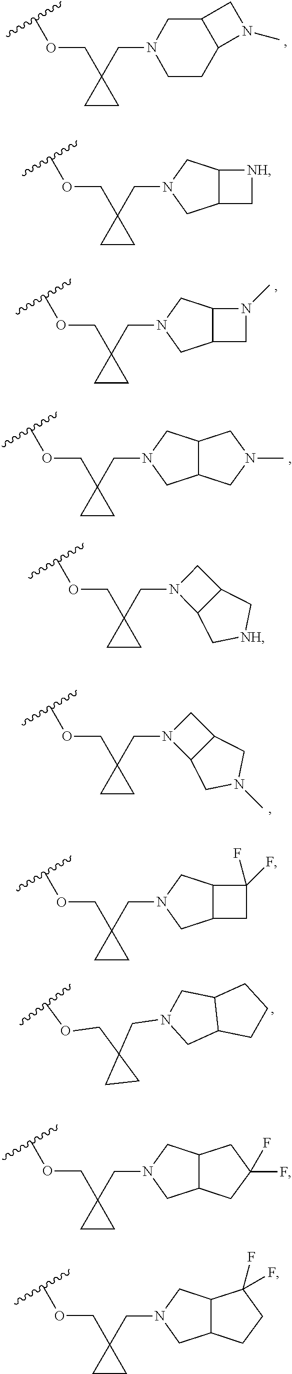

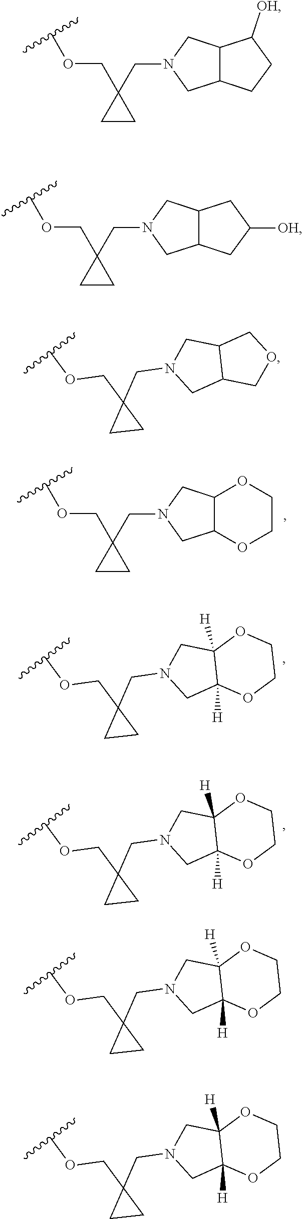

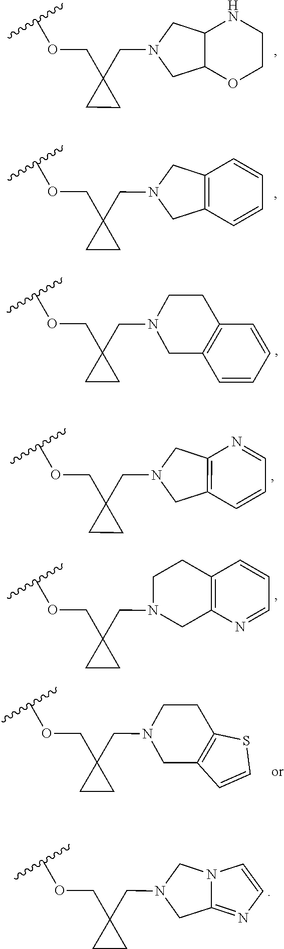

[82]. The compound according to any one of [1] to [2], wherein, the compound of formula (I) is selected from formula (I-D):

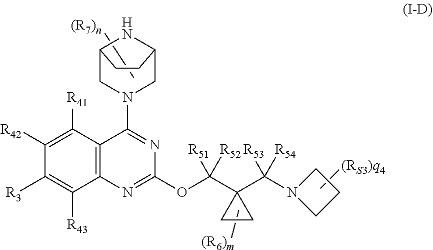

wherein,
R_{S4} at each occurrence is independently selected from —F, —Cl, —Br, —C₁₋₃alkyl, —NH₂, —OH, —O(C₁₋₃alkyl); preferably, R_{S4} at each occurrence is independently selected from —F, —Cl, —CH₃, —CH₂CH₃, —CH₂CH₂CH₃, —CH(CH₃)₂, —NH₂, —OH, or —OCH₃;
q₄ is selected from 0, 1, or 2.

[83]. The compound according to [1], [2], or [82], wherein, the moiety of

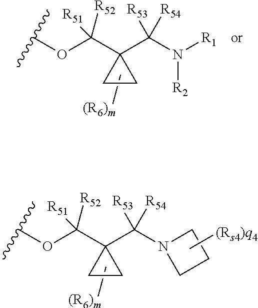

in the formula (I-D) is independently selected from

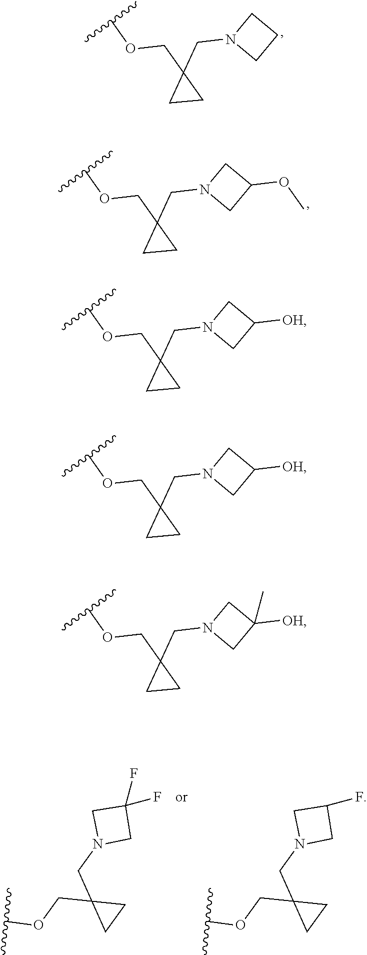

[84]. The compound according to any one of [1] to [2], wherein, the compound of formula (I) is selected from formula (I-E):

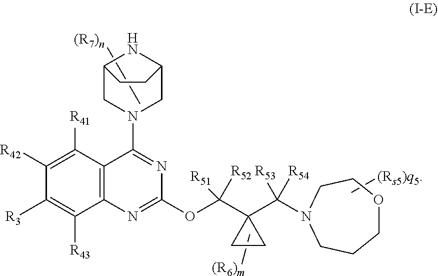

[85]. The compound according to [1], [2] or [84], wherein, the moiety of

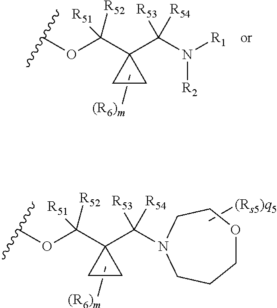

in formula (I-E) is selected from

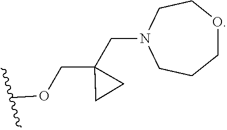 [SMILES: *OCC1(CN2CCCOCC2)CC1]

[86]. The compound according to any one of [1] to [2], wherein, the compound of formula (I) is selected from formula (I-F):

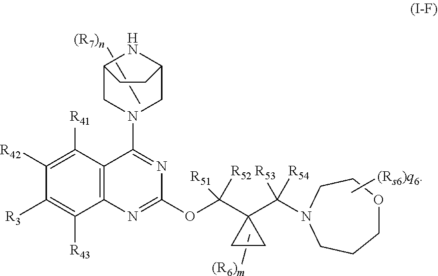

[87]. The compound according to [1], [2] or [85], wherein the moiety of

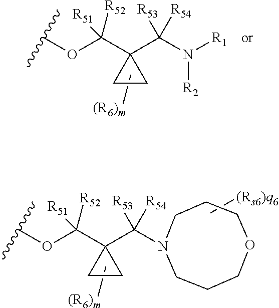

in formula (I-F) is selected from

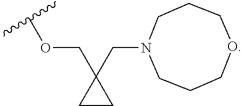 [SMILES: *OCC1(CN2CCCOCCC2)CC1]

[88]. The compound according to [1] to [87], wherein, R₁ and R₂ together with the nitrogen atom to which they are both attached form

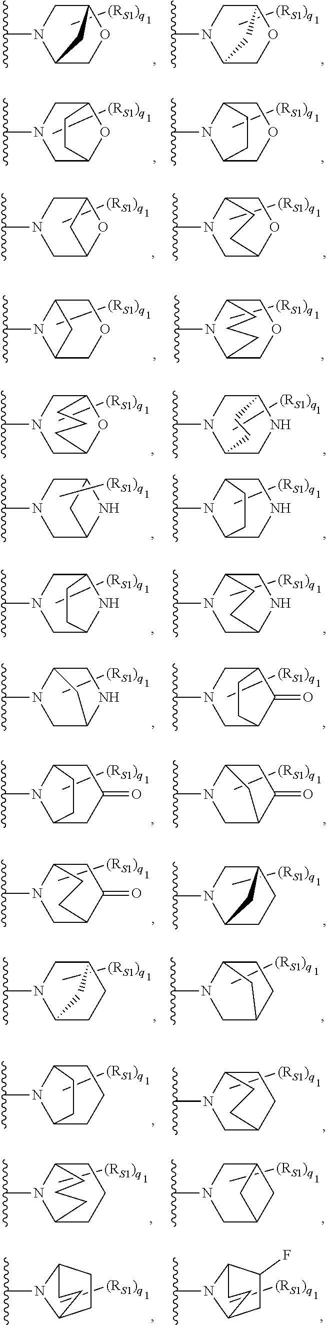

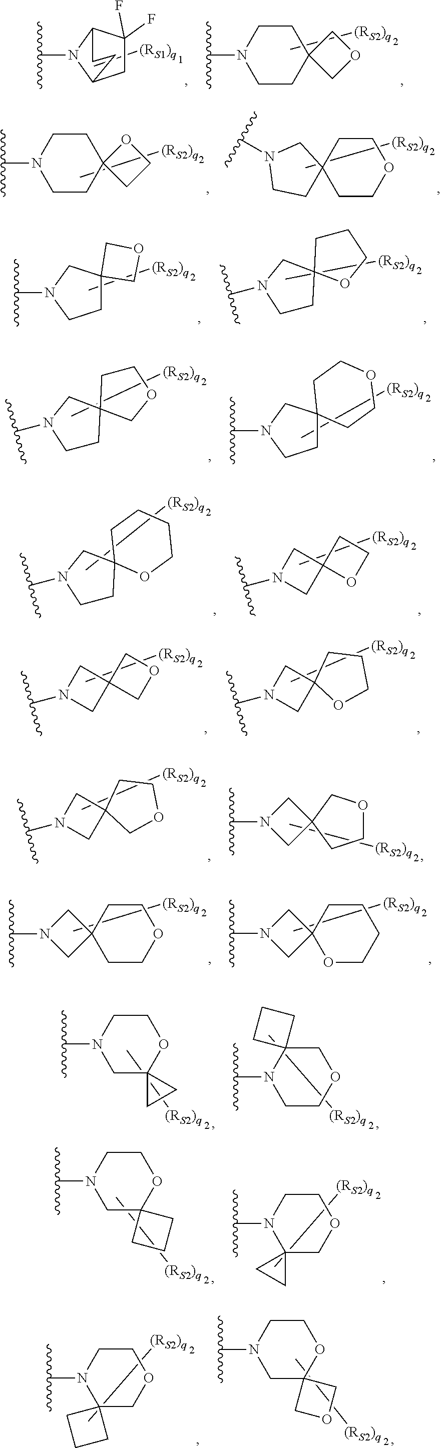

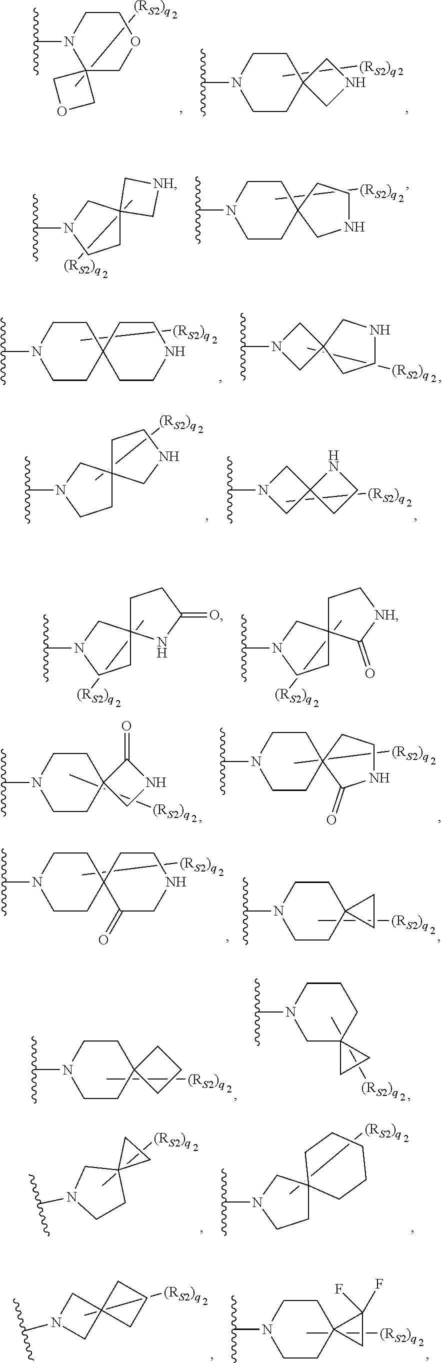

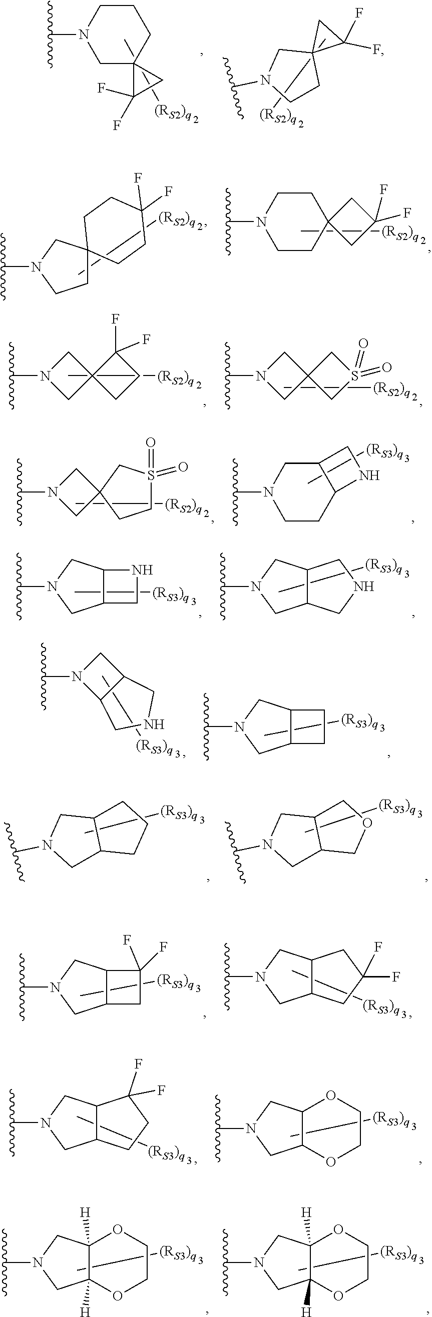

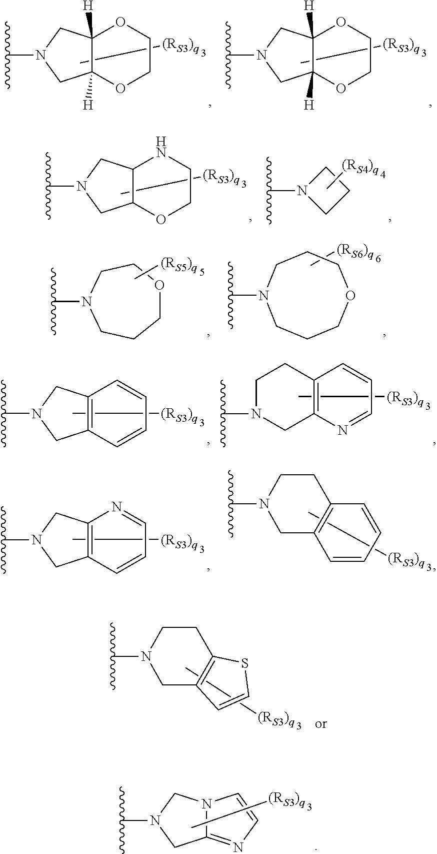

[89]. The compound according to any one of [1] to [88], the moiety of

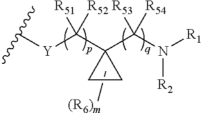

is selected from

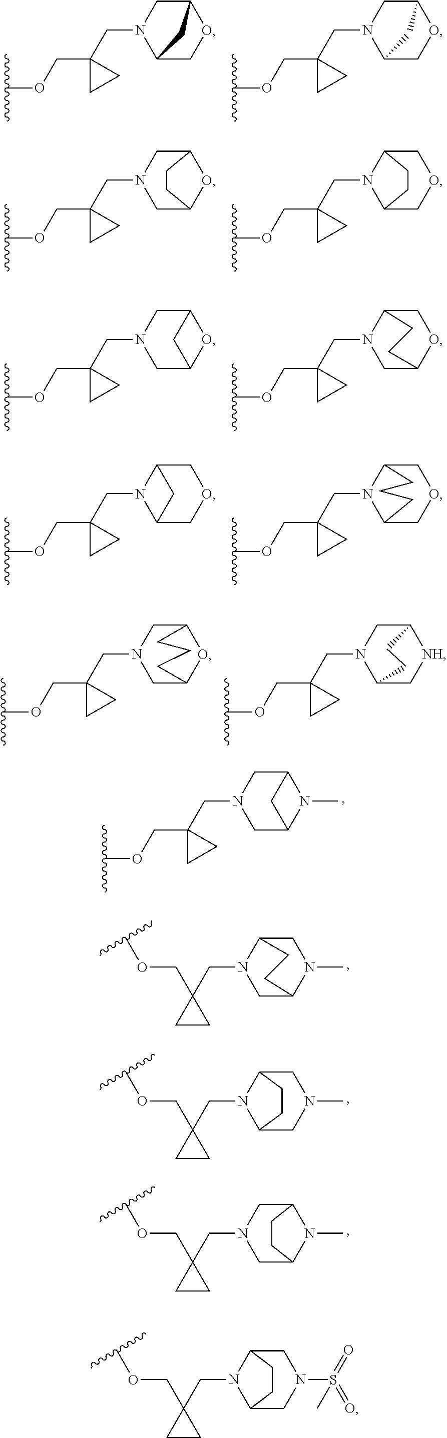

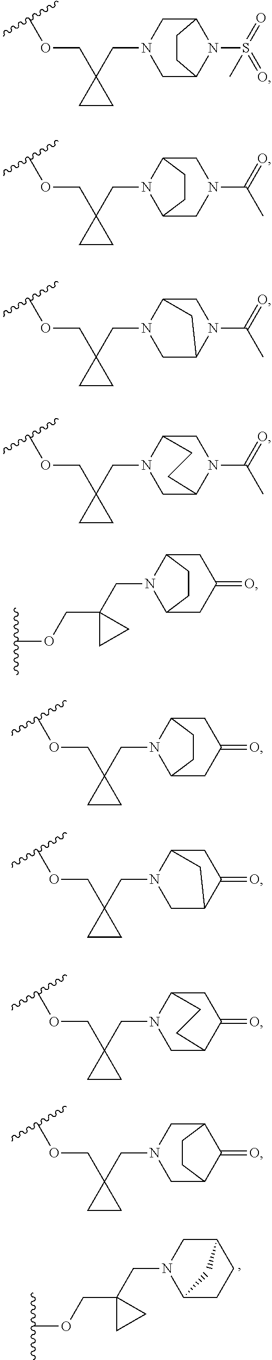

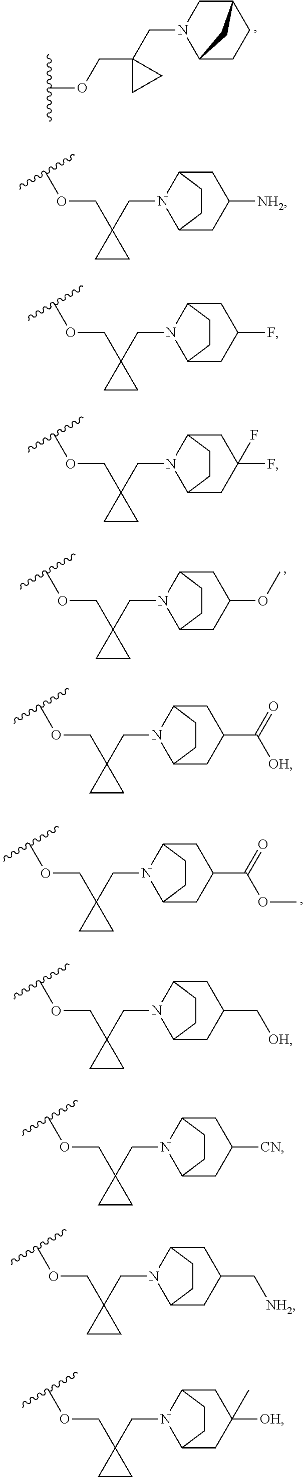

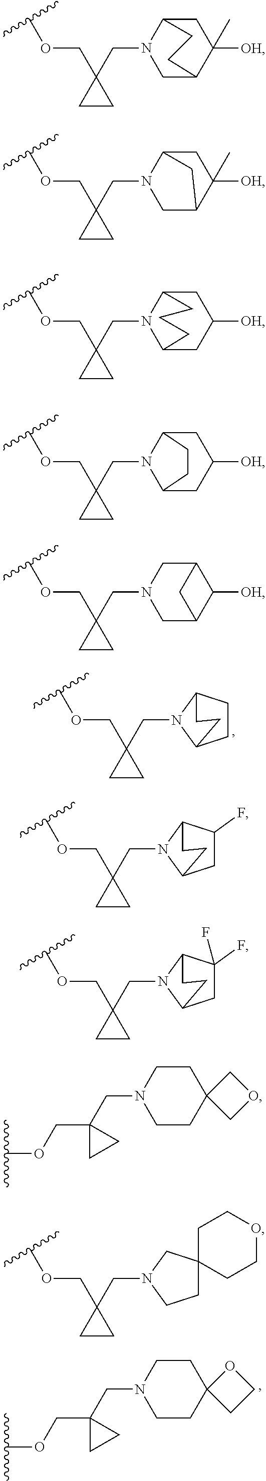

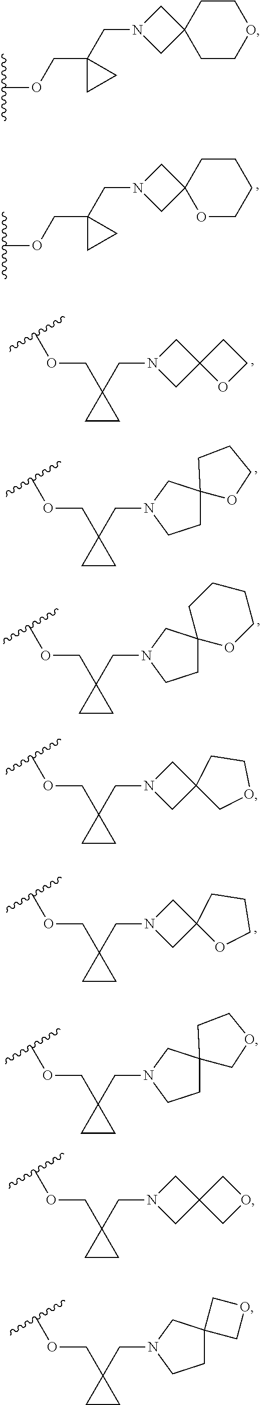

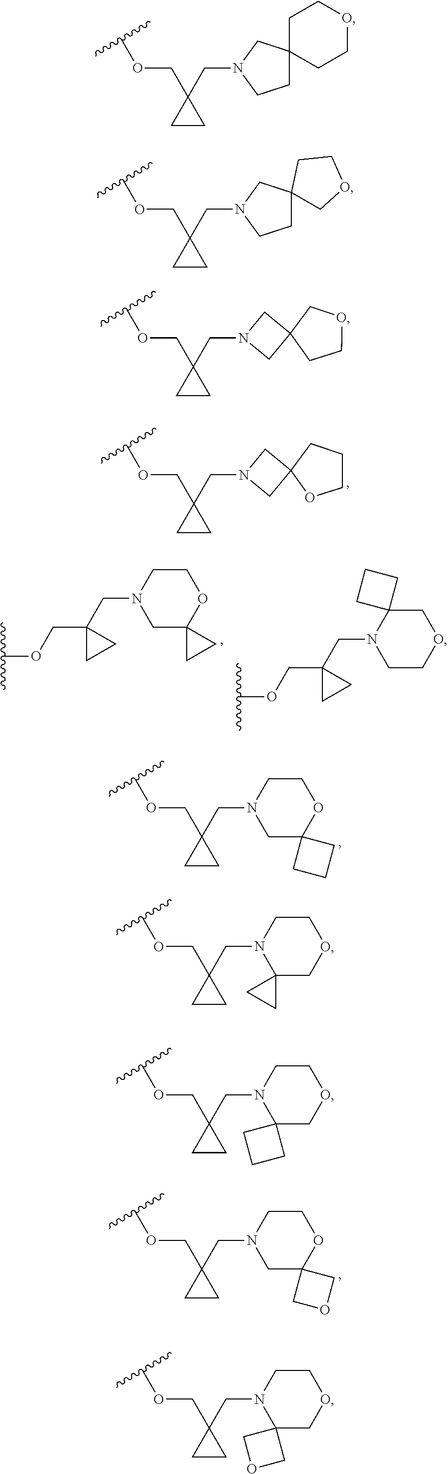

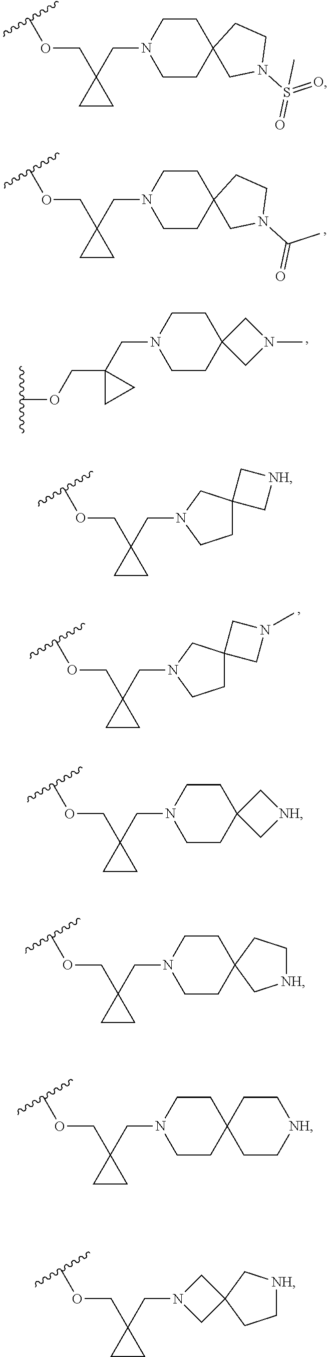

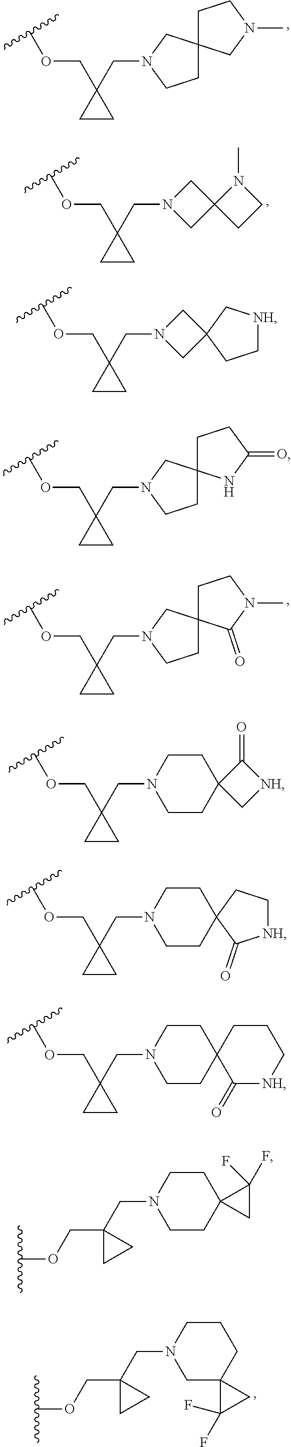

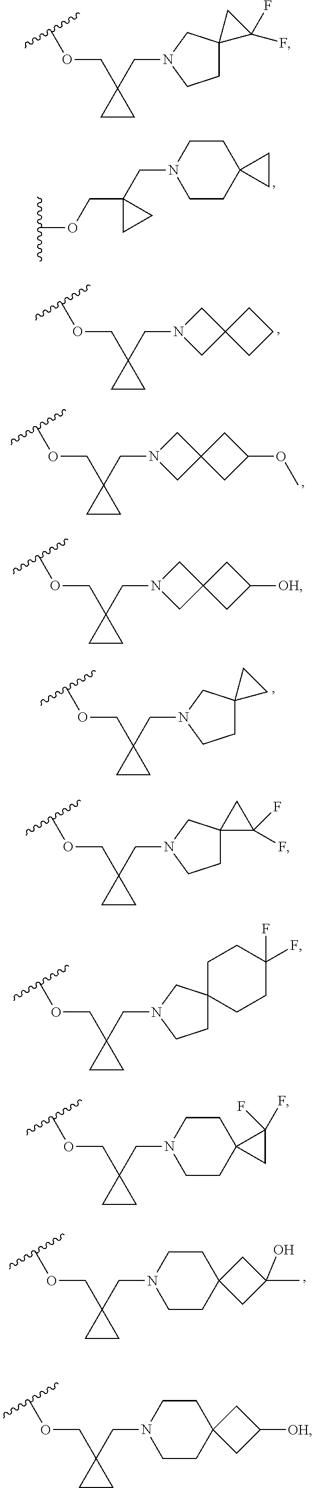

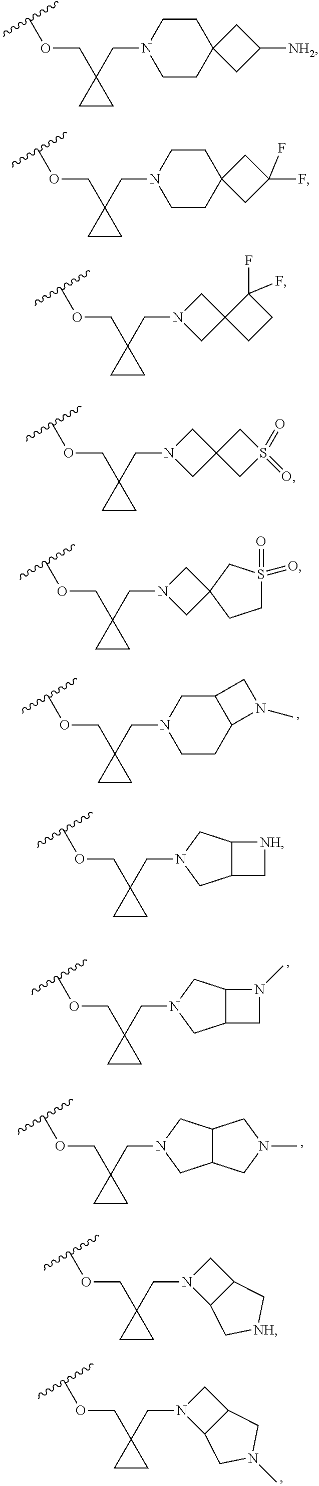

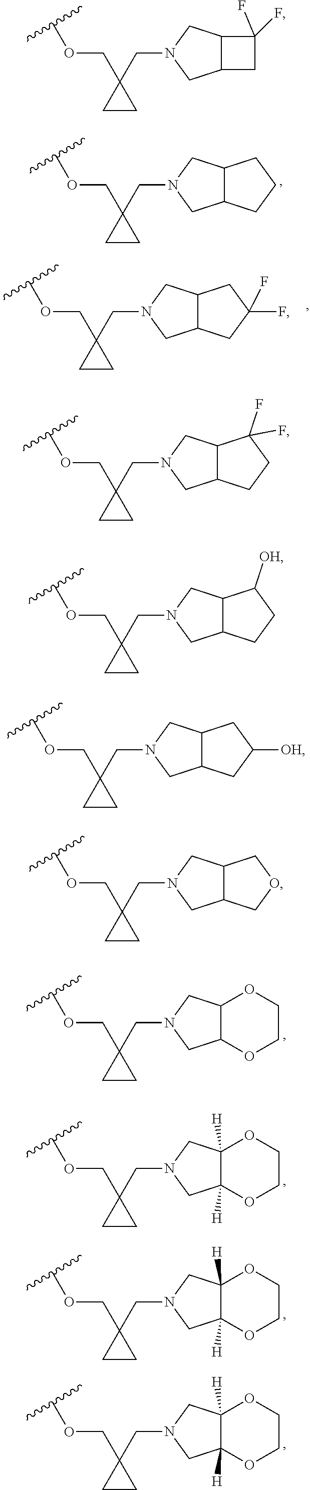

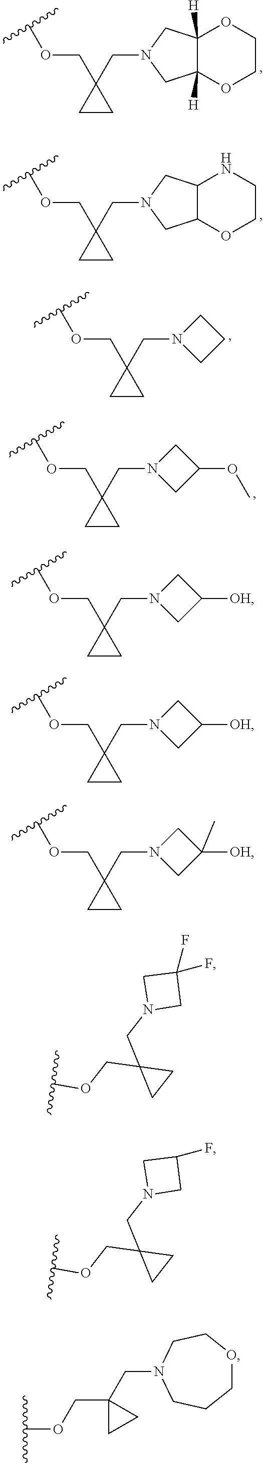

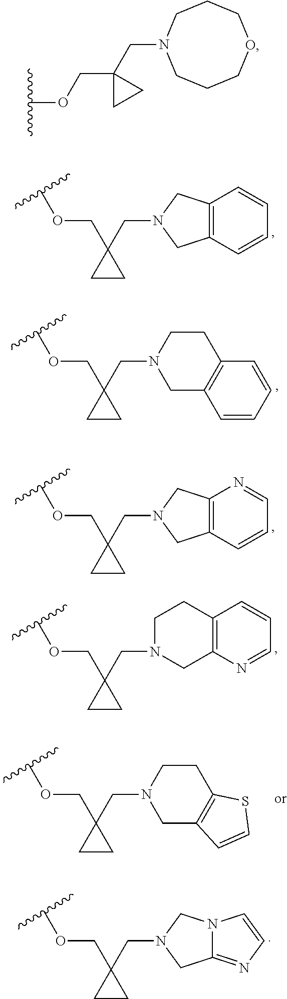

[90]. The compound according to any one of [1] to [89], wherein, the compound of formula (I) is selected from any one of the following formulas:

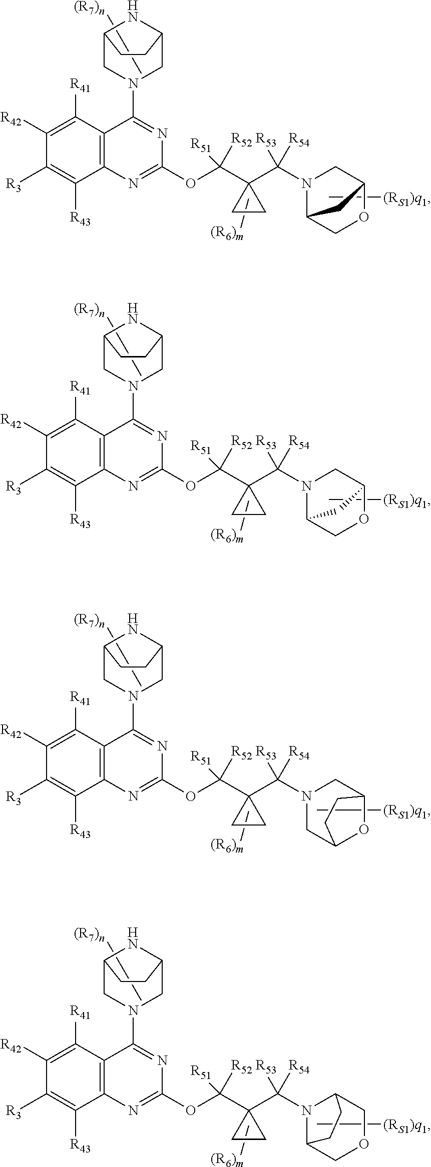

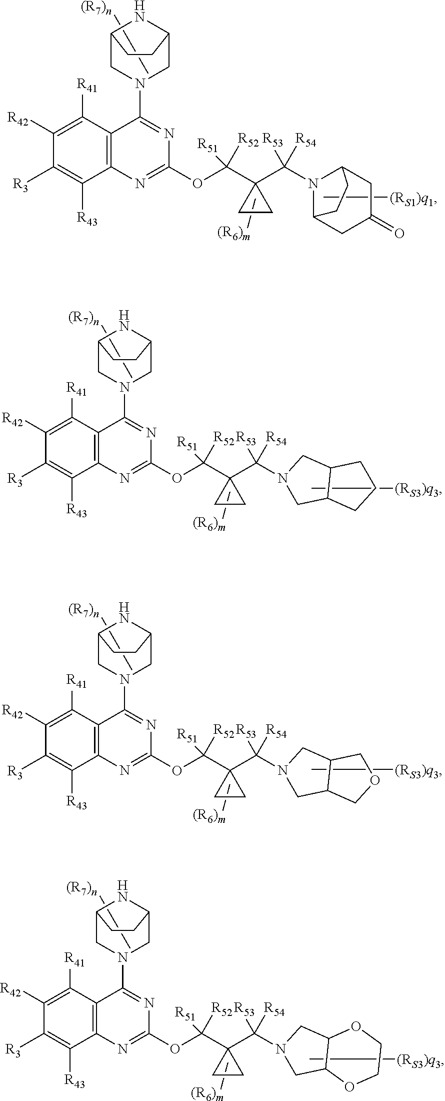

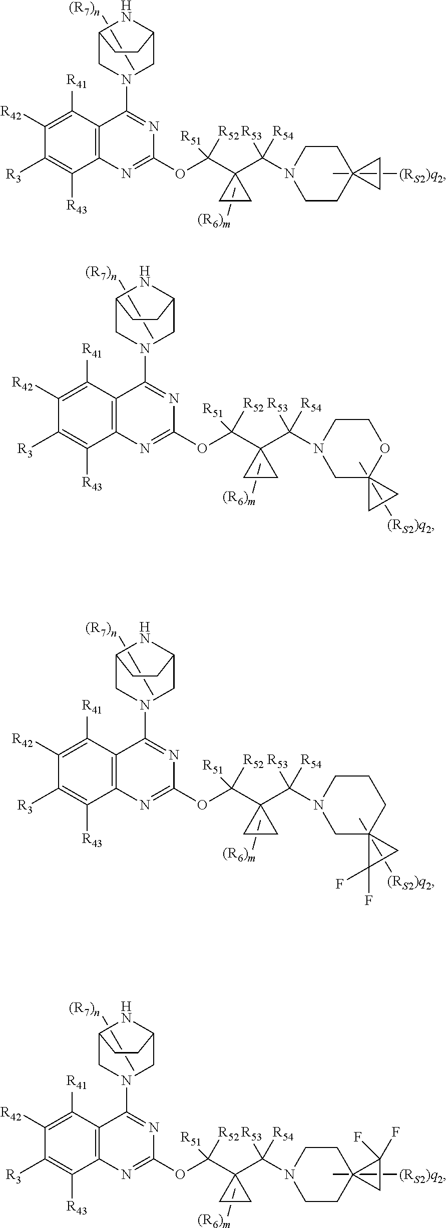

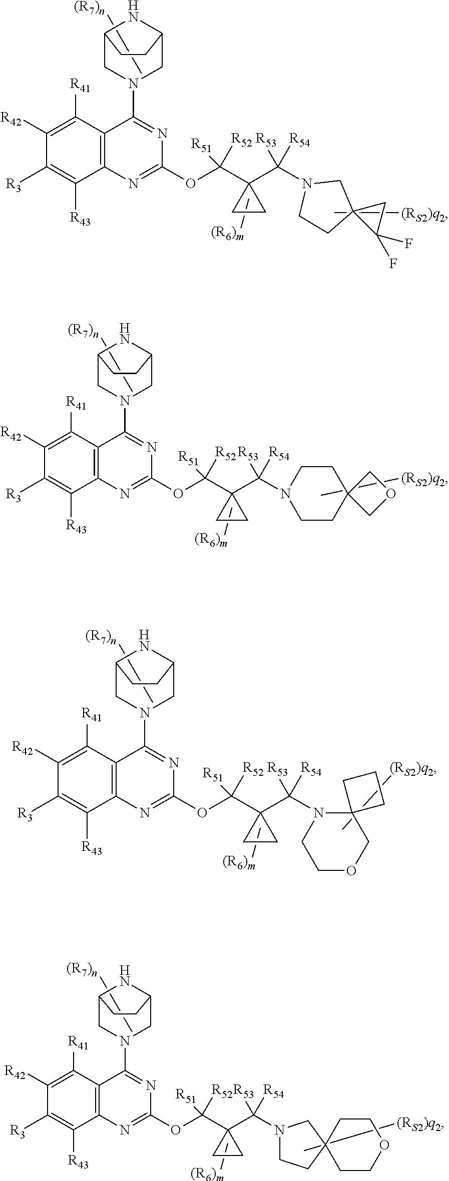

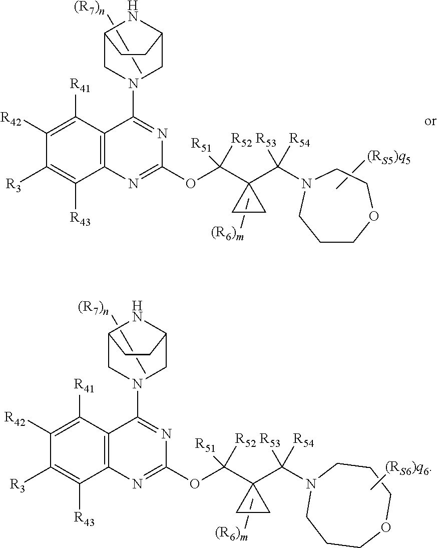

[91]. The compound according to any one of [1] to [90], wherein, R₃ is selected from:

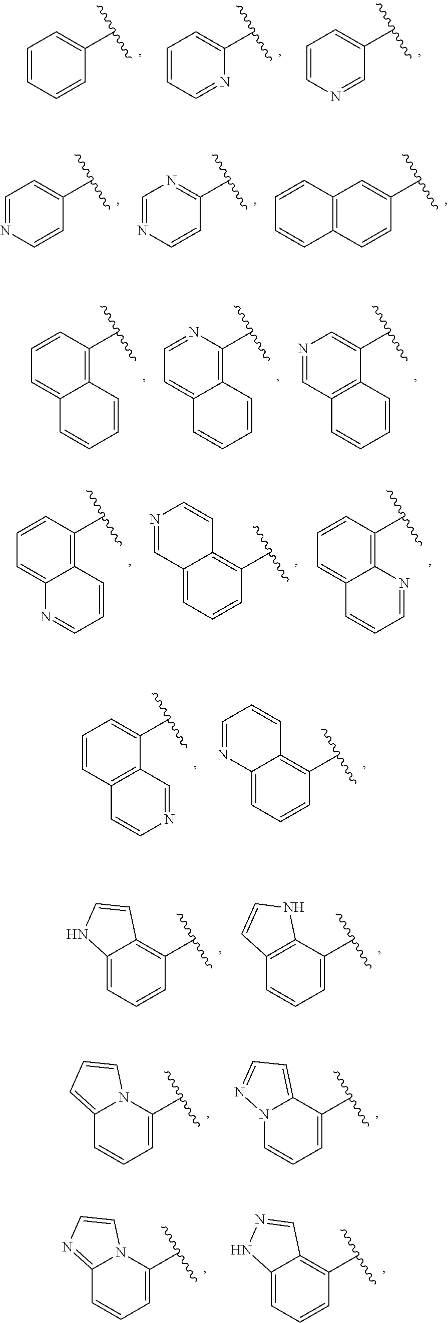

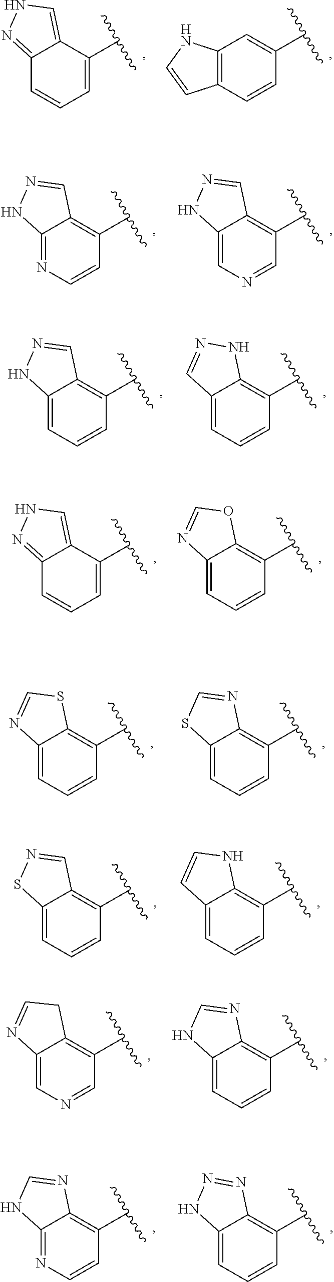

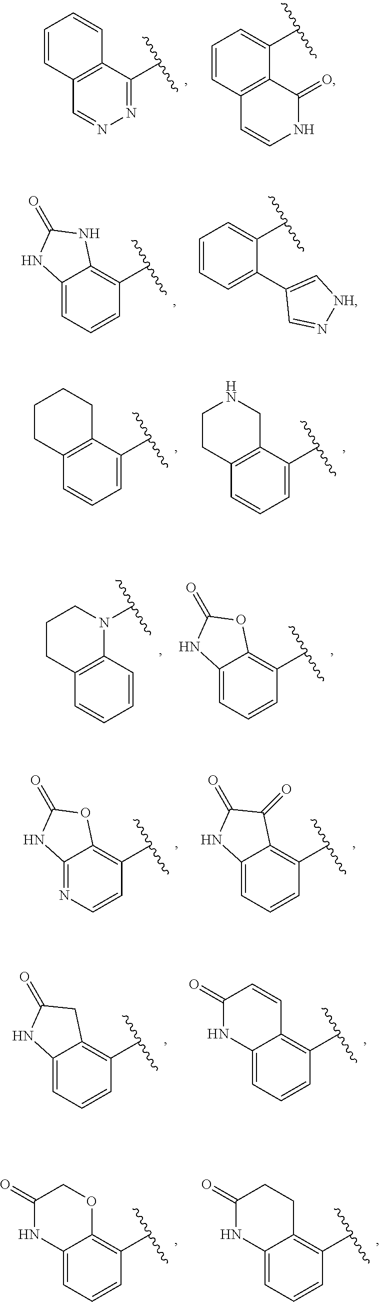

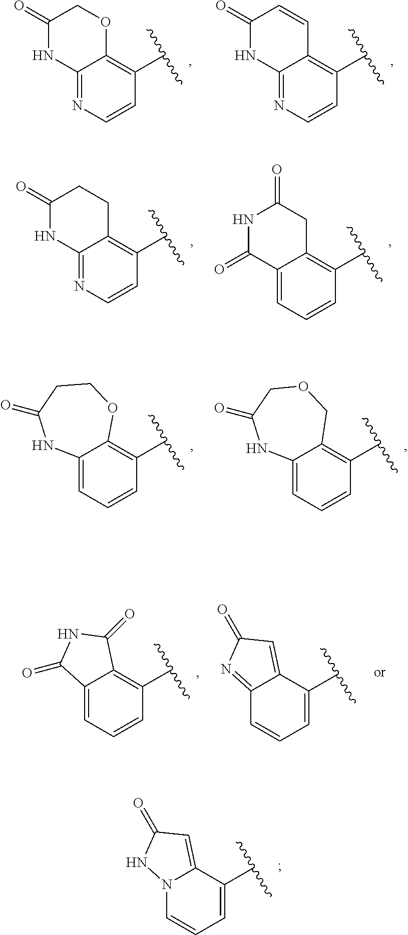

Each of which is independently optionally substituted with 1, 2, 3, 4, 5 or 6 R₃₁.

[92]. The compound according to any one of [1] to [91], wherein, R₃ is selected from

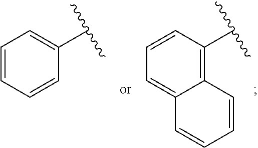 [SMILES: *c1ccccc1 ; *c1cccc2ccccc12]

each of which is independently optionally substituted with 1, 2, 3, 4, 5 or 6 R₃₁.

[93]. The compound according to any one of [1] to [92], wherein, R₃₁ at each occurrence is independently selected from halogen, —C₁₋₄alkyl, —C₂₋₆alkenyl, —C₂₋₆alkynyl, —C₁₋₃haloalkyl, —C₁₋₃haloalkoxy, —CN, —NH₂, —NH(C₁₋₃alkyl), —N(C₁₋₃alkyl)₂, —OH, —O(C₁₋₃alkyl), —SH, —S(C₁₋₃alkyl), —S(C₁₋₃haloalkyl), —S(═O)₂C₁₋₃alkyl, —C(═O)C₁₋₃alkyl, —C(═O)NH₂, —C(═O)NHC₁₋₃alkyl, —C(═O)N(C₁₋₃alkyl)₂, —S(═O)₂NH₂, —S(═O)₂NHC₁₋₃alkyl, —S(═O)₂N(C₁₋₃alkyl)₂, 3 membered cycloalkyl, 4 membered cycloalkyl, 5 membered cycloalkyl, or 6 membered cycloalkyl, wherein, said —C₁₋₄alkyl, —C₂₋₆alkenyl, —C₂₋₆alkynyl, —C₁₋₃haloalkyl, —C₁₋₃haloalkoxy, 3 membered cycloalkyl, 4 membered cycloalkyl, 5 membered cycloalkyl or 6 membered cycloalkyl is optionally substituted with 1 or 2 substituents selected from halogen, —C₁₋₃haloalkoxy, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)₂C₁₋₃alkyl, —C(═O)C₁₋₃alkyl, —C(═O)NH₂, —C(═O)NHC₁₋₃alkyl, —C(═O)N(C₁₋₃alkyl)₂, —S(═O)₂NH₂, —S(═O)₂NHC₁₋₃alkyl, —S(═O)₂N(C₁₋₃alkyl)₂, or 3-6 membered cycloalkyl.

[94]. The compound according to any one of [1] to [93], wherein, R₃₁ at each occurrence is independently selected from —F, —Cl, —Br, —CH₃, —CH₂CH₃, —CH(CH₃), —CH₂CH₂CH₃, —CHF₂, —CF₃, —CH₂CF₃, —CH₂CHF₂, —CH₂CH₂F, —CH₂CH₂CH₂F, —OCF₃, —CN, —CH₂CN, —CH₂CH₂CN, —NH₂, —N(CH₃)₂, —NHCH₂CH₃, —CH₂—N(CH₃)₂, —OH, —CH₂OH, —CH₂C(═O)NH₂, —CH₂CH₂OH, —OCH₃, —OC(CH₃)₂, —CH₂CH(CH₃)₂, —CH(CH₃)CH₂CH₃, —CH₂OCH₃, —SH, —SCH₃, —SCF₃, —OCHF₂, —CH(CF₃)OCH₃, —C(CH₃)₂OH, —CF(CH₃)₂, —OCH(CH₃)₂, cyclopropyl,

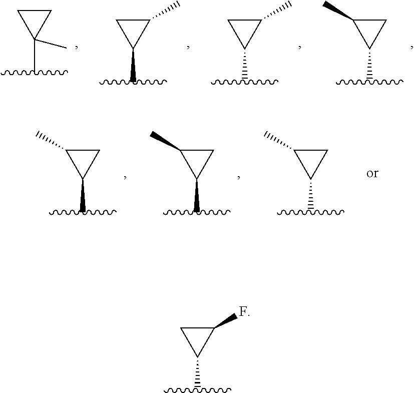

[95]. The compound according to any one of [1] to [94], wherein, R₃ is selected from:

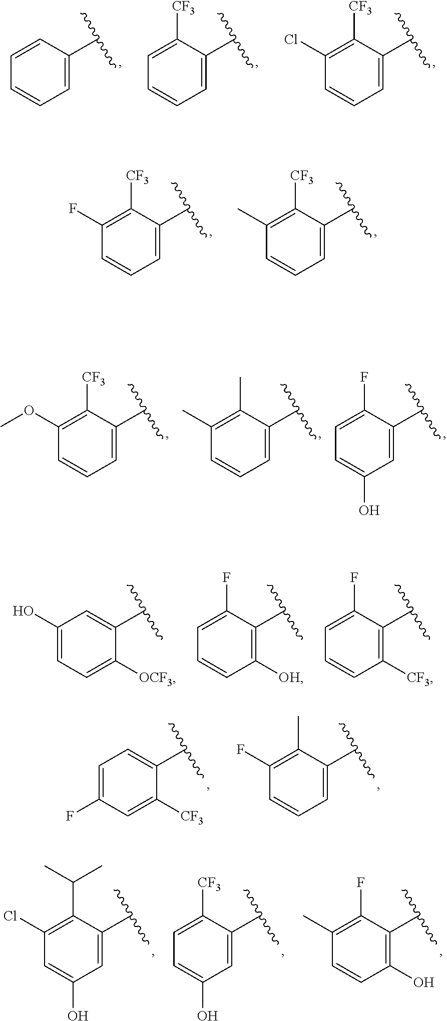

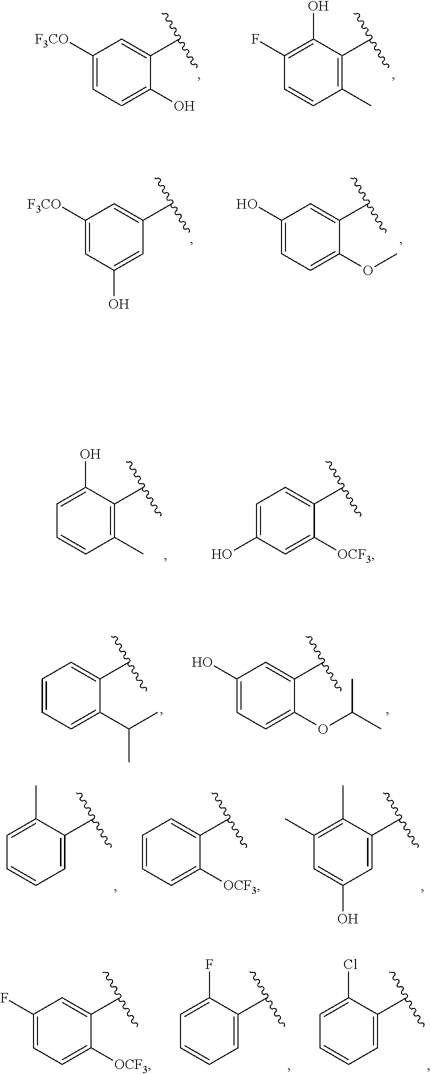

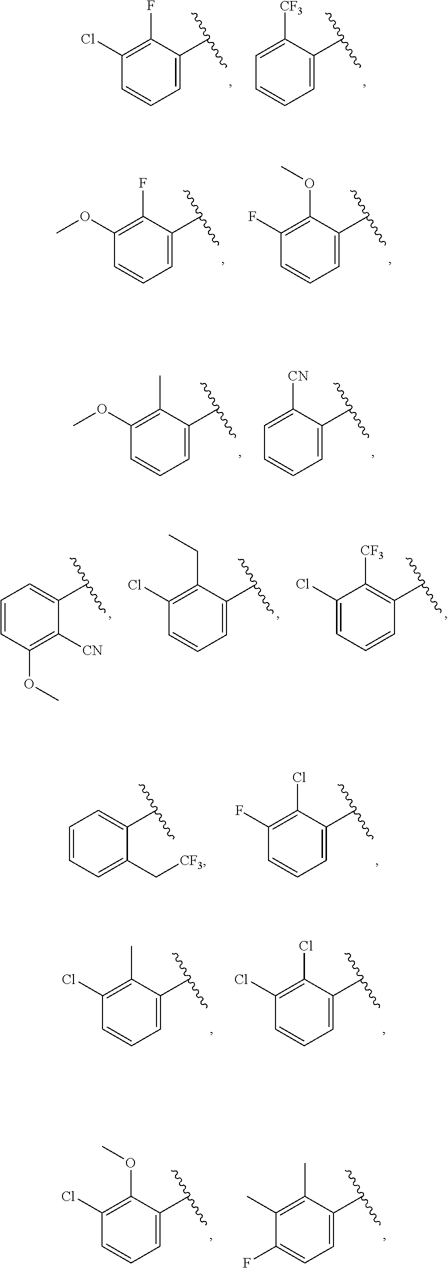

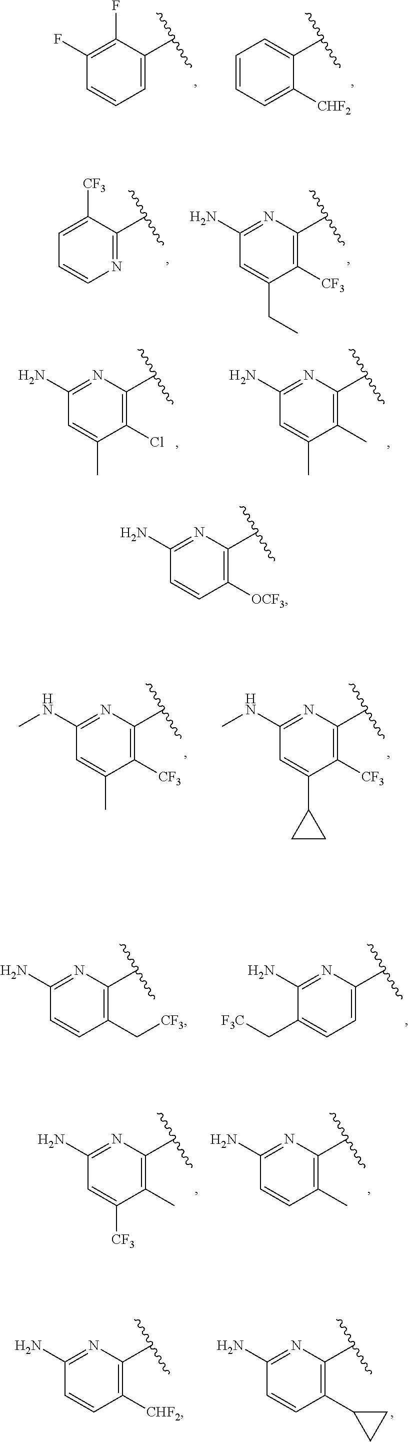

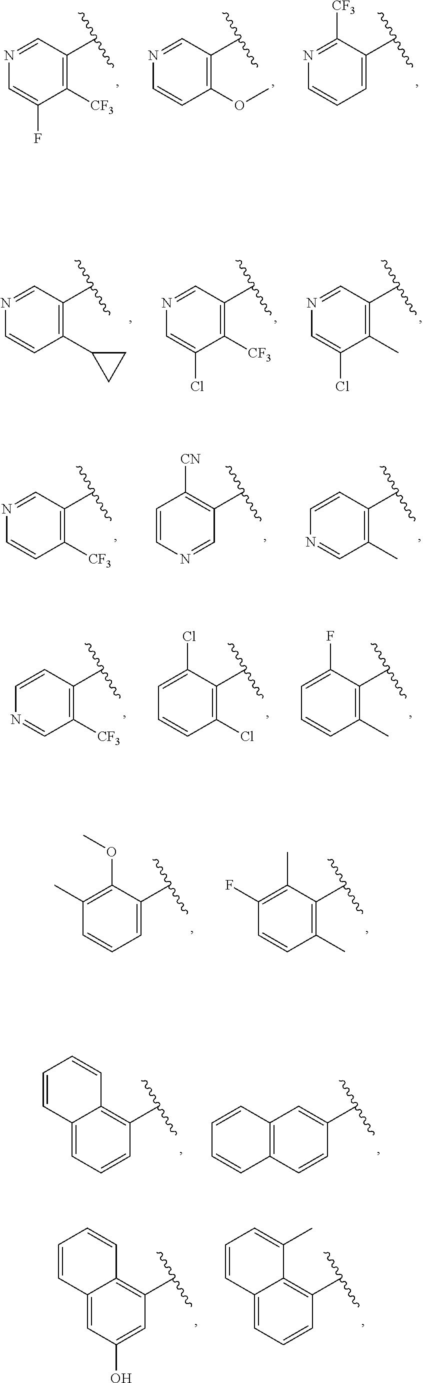

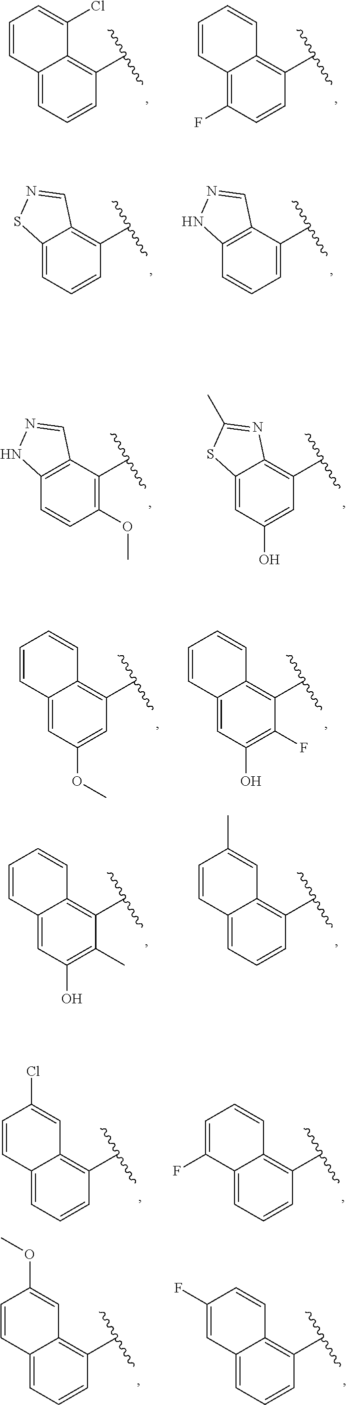

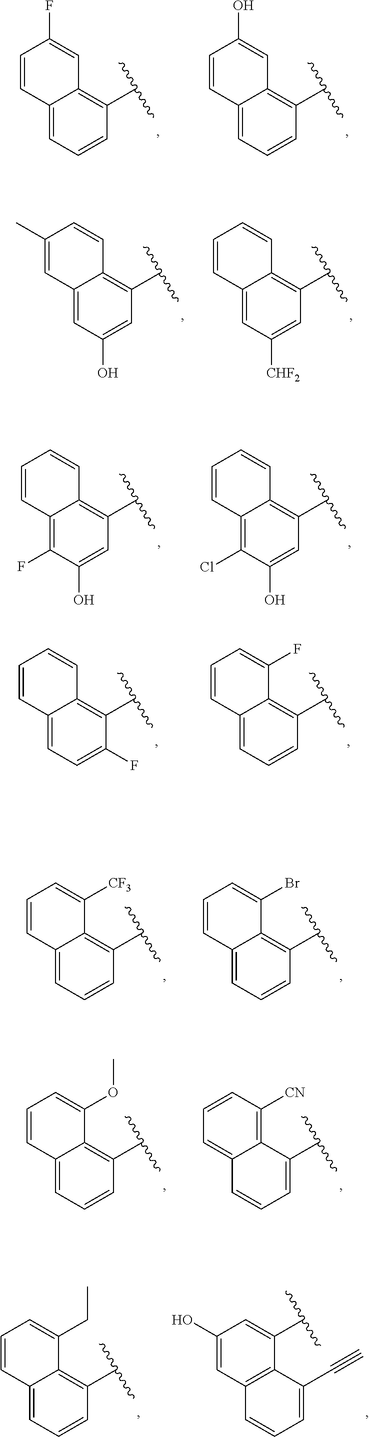

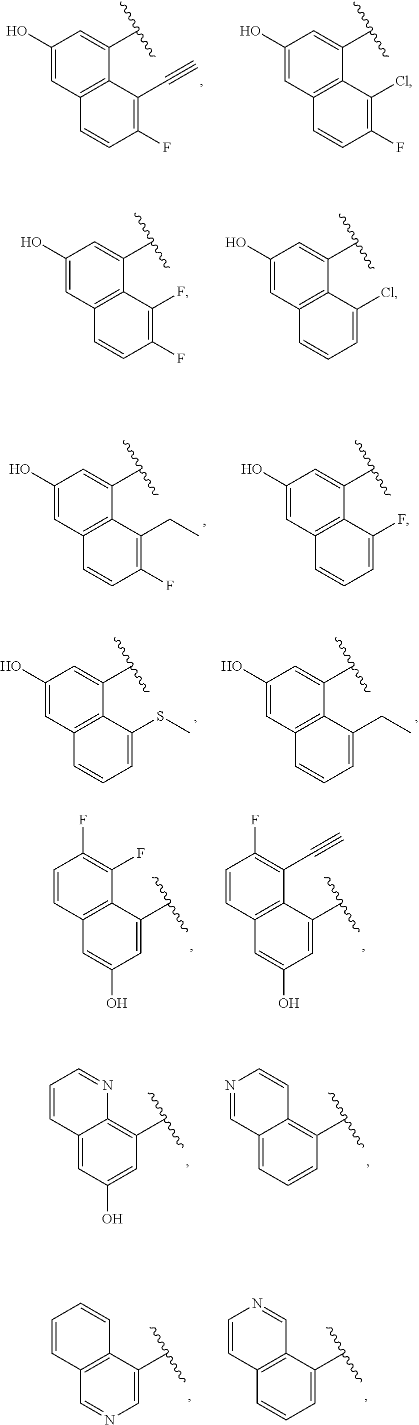

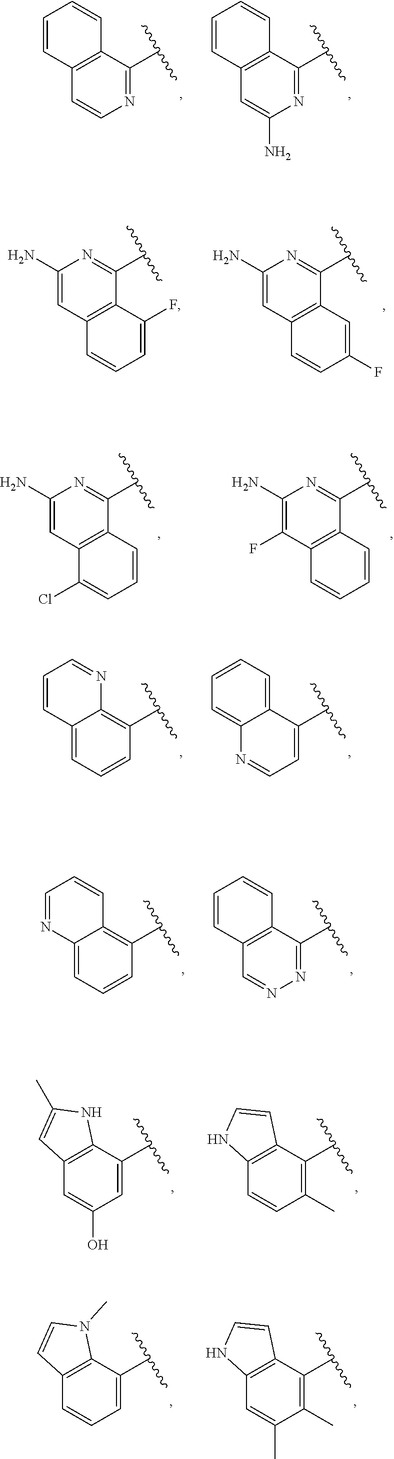

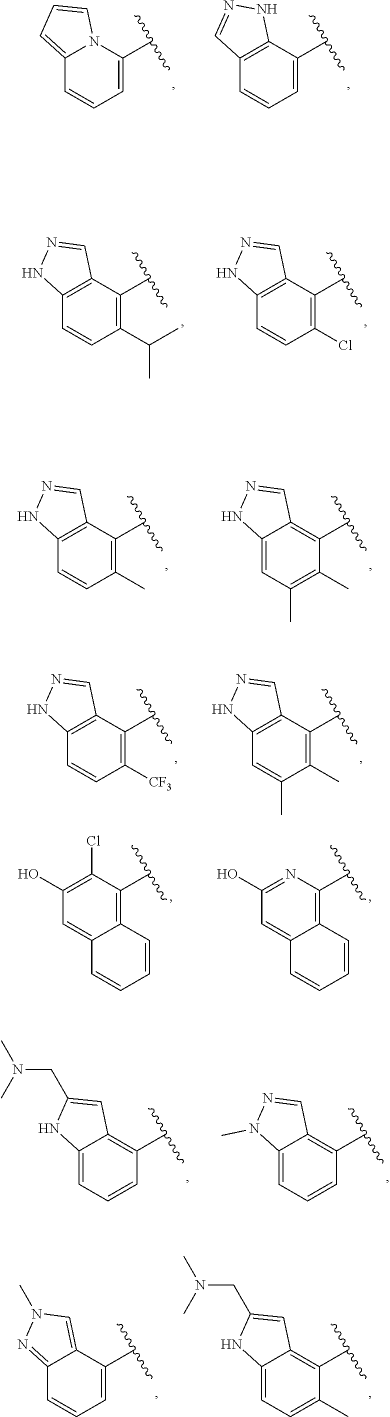

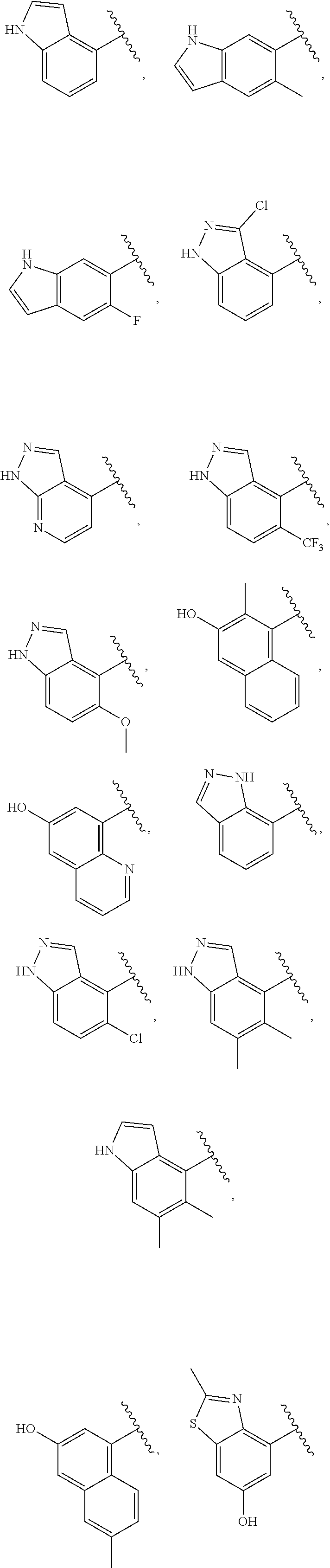

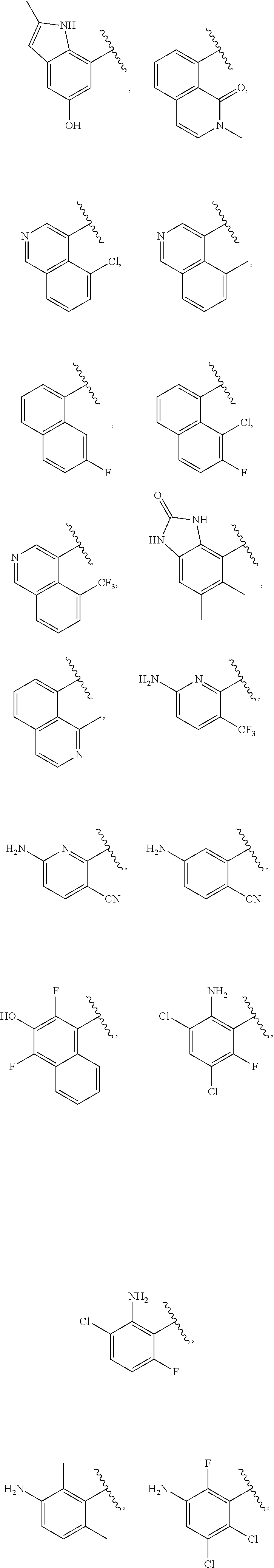

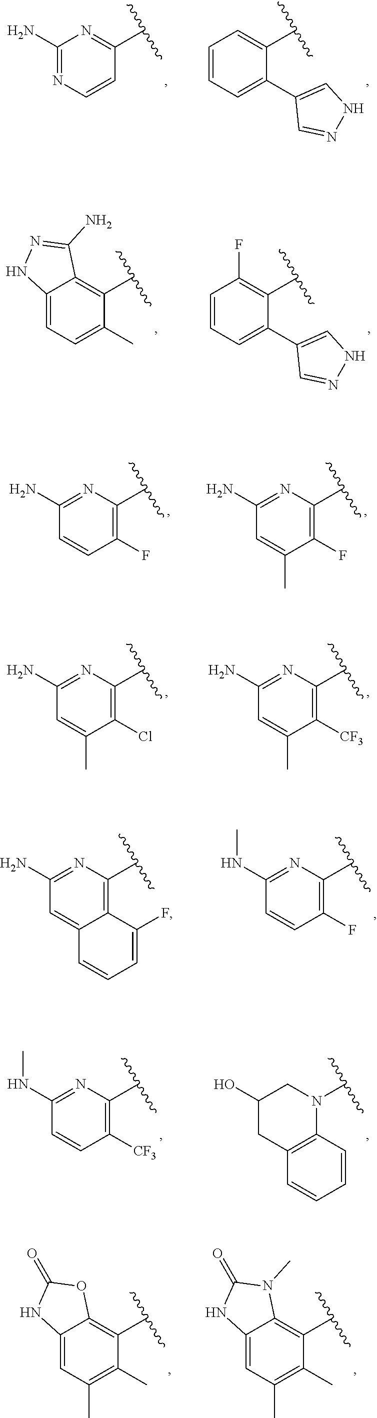

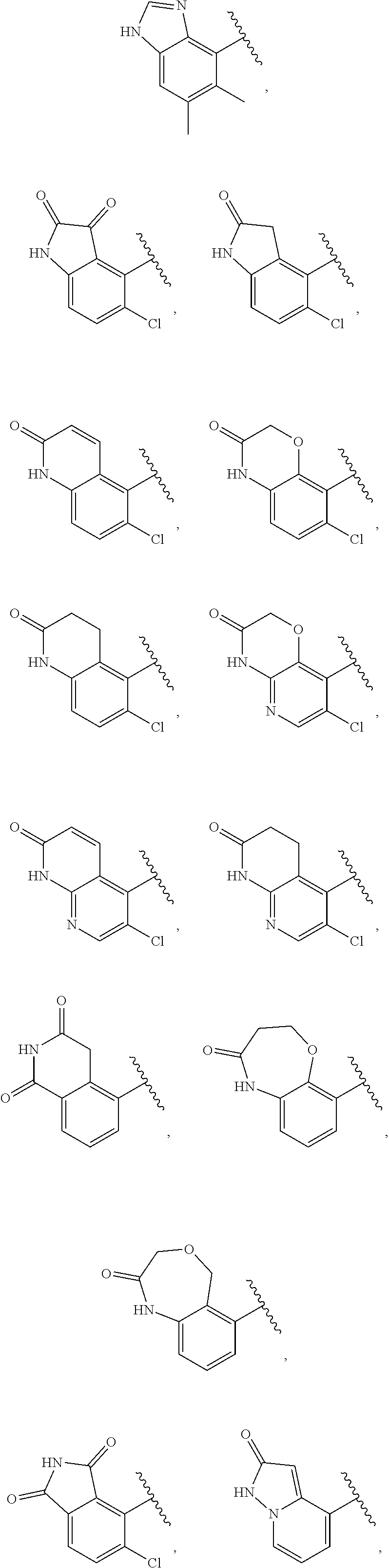

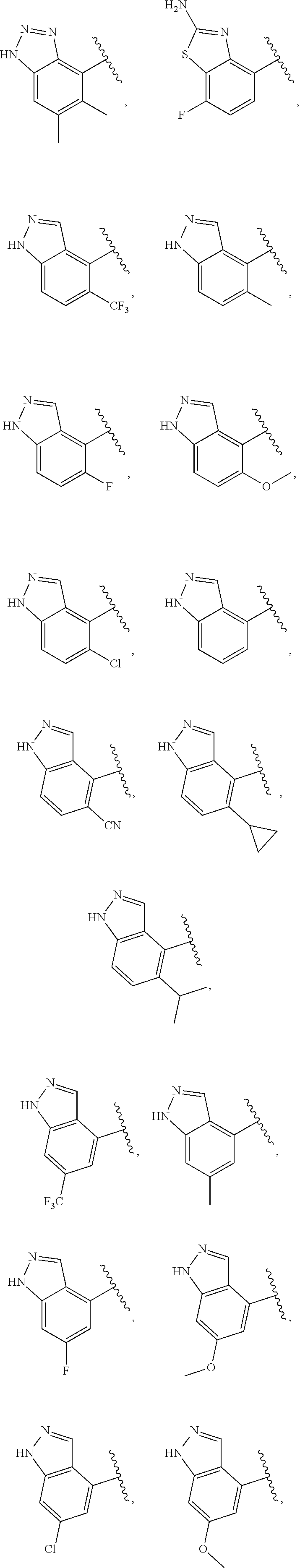

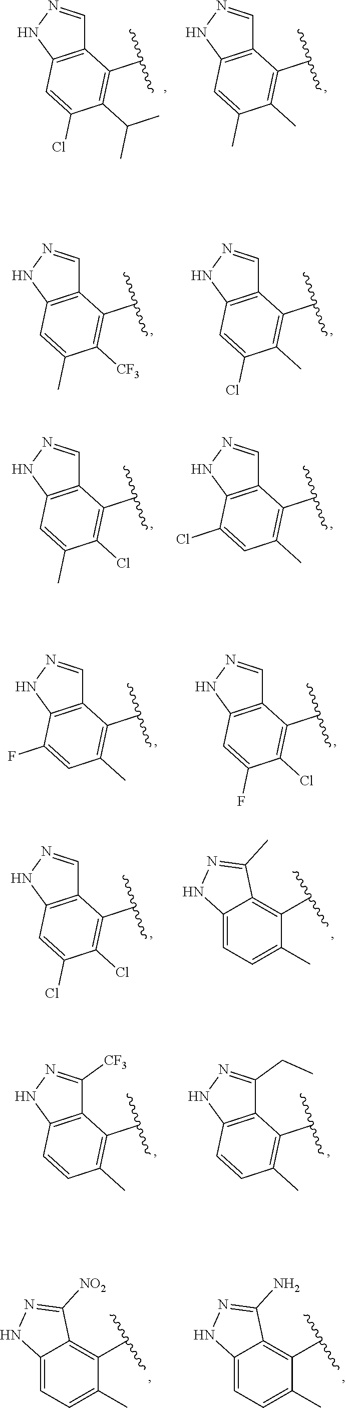

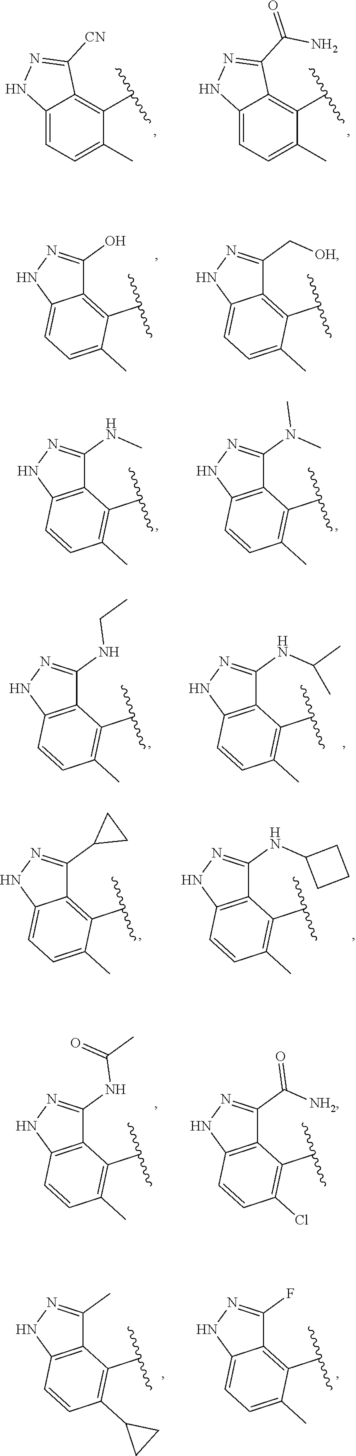

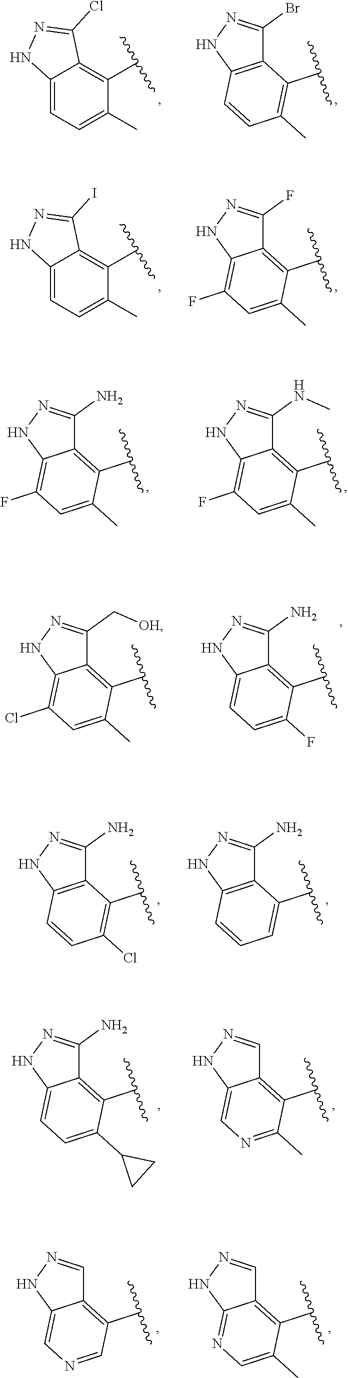

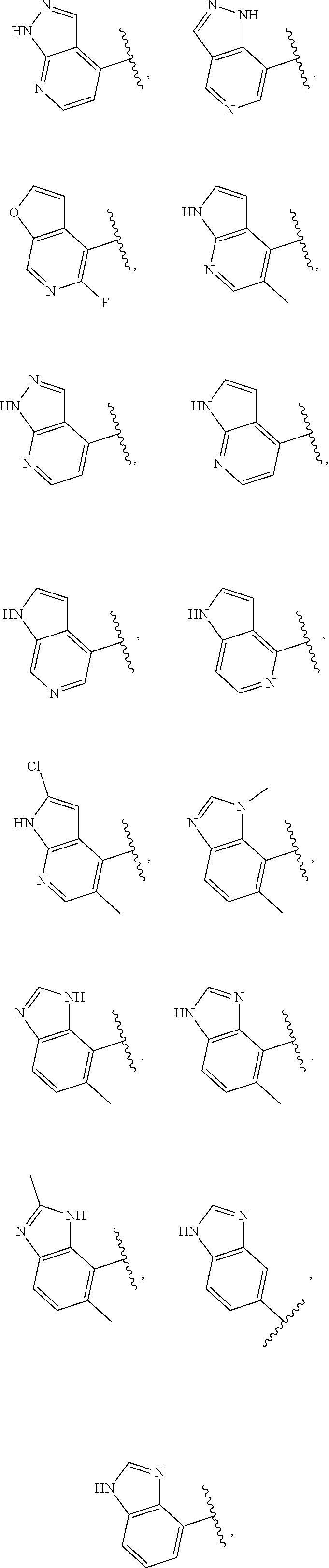

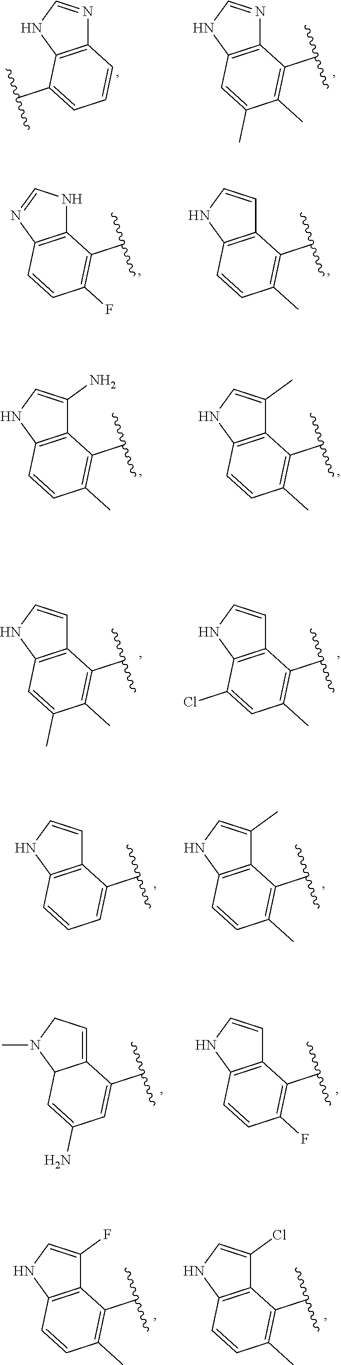

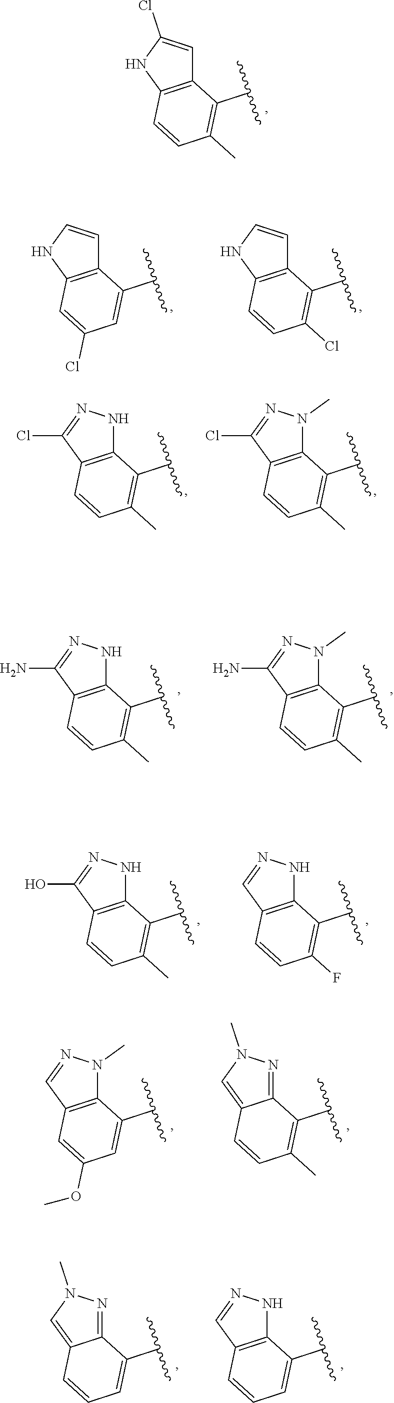

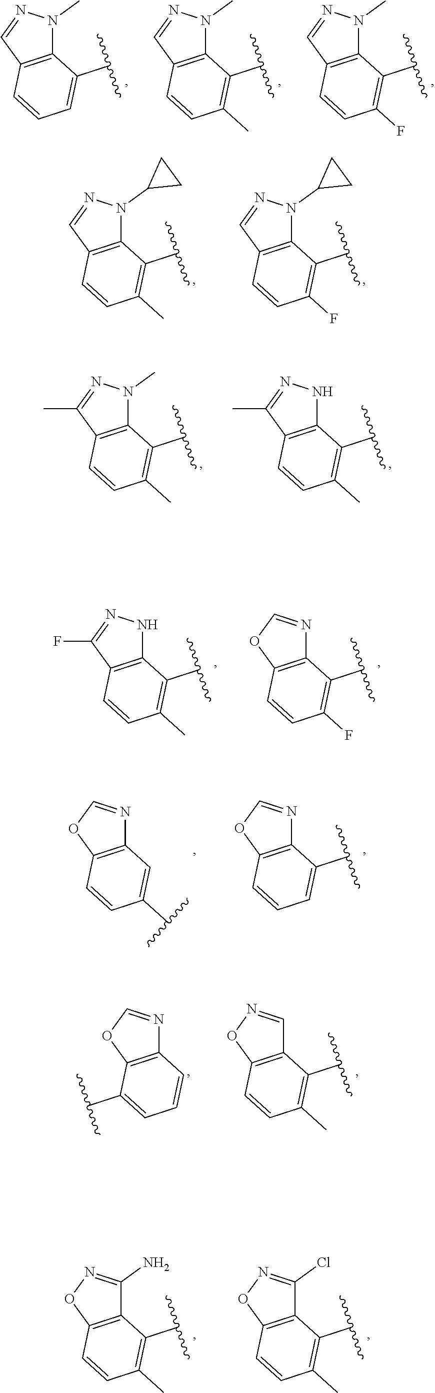

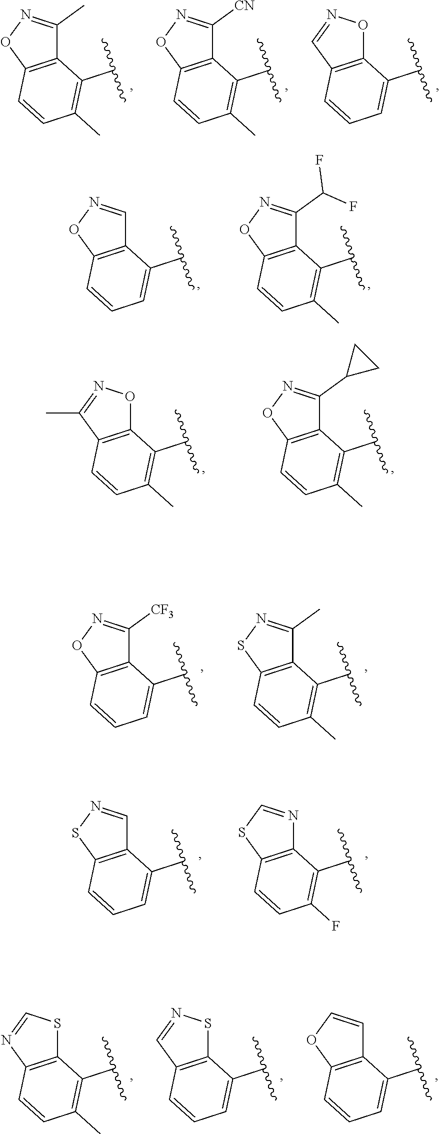

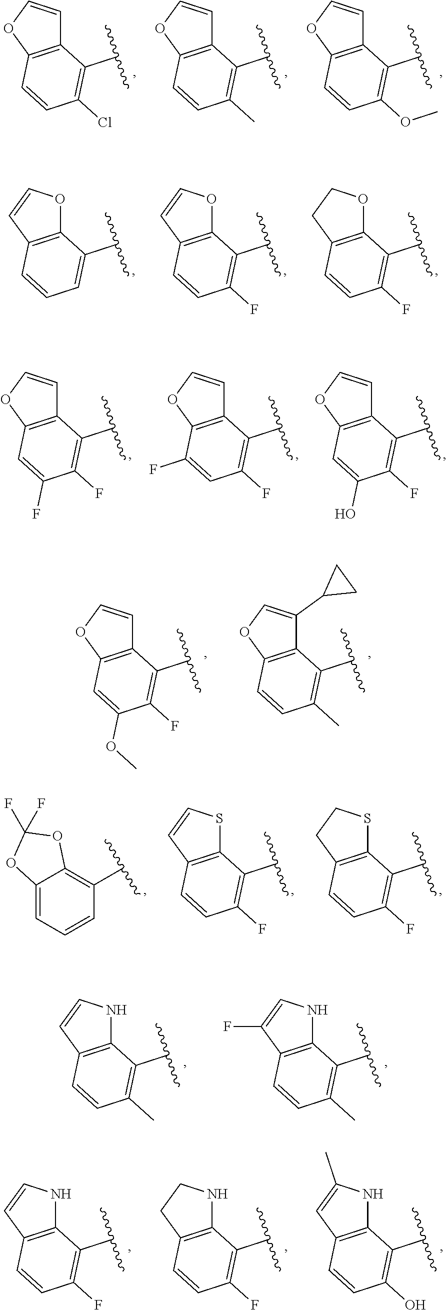

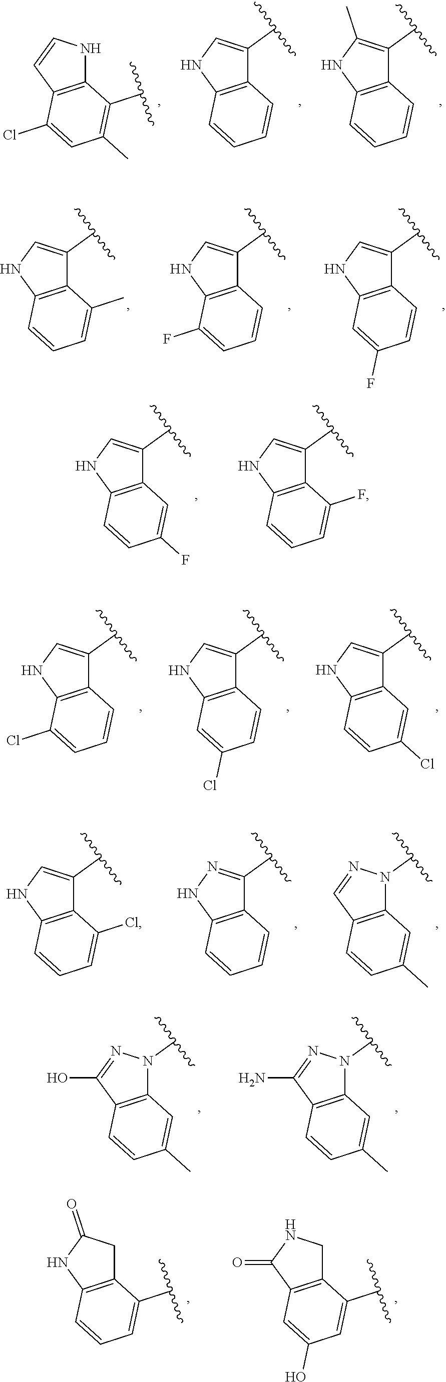

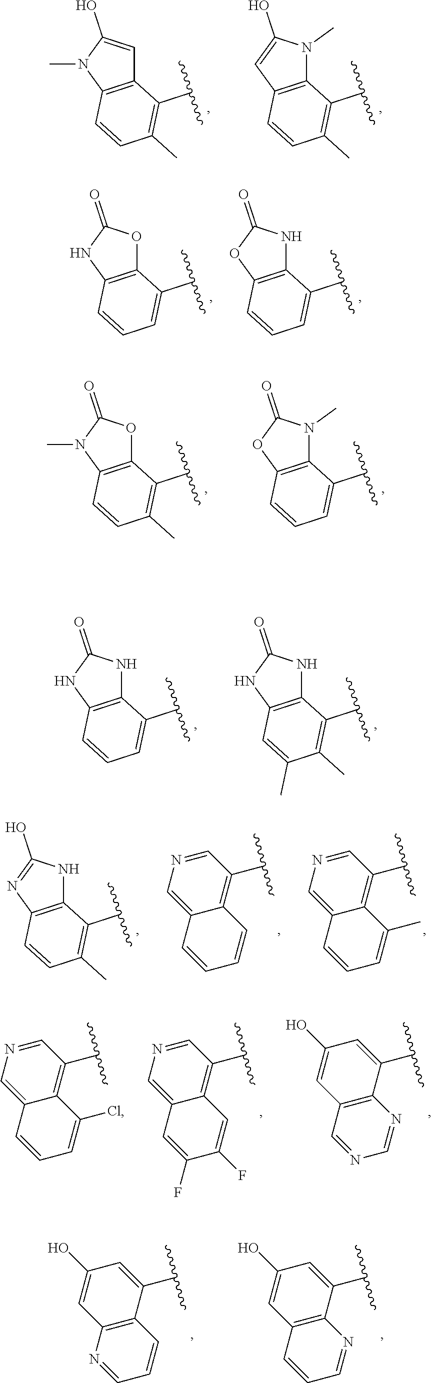

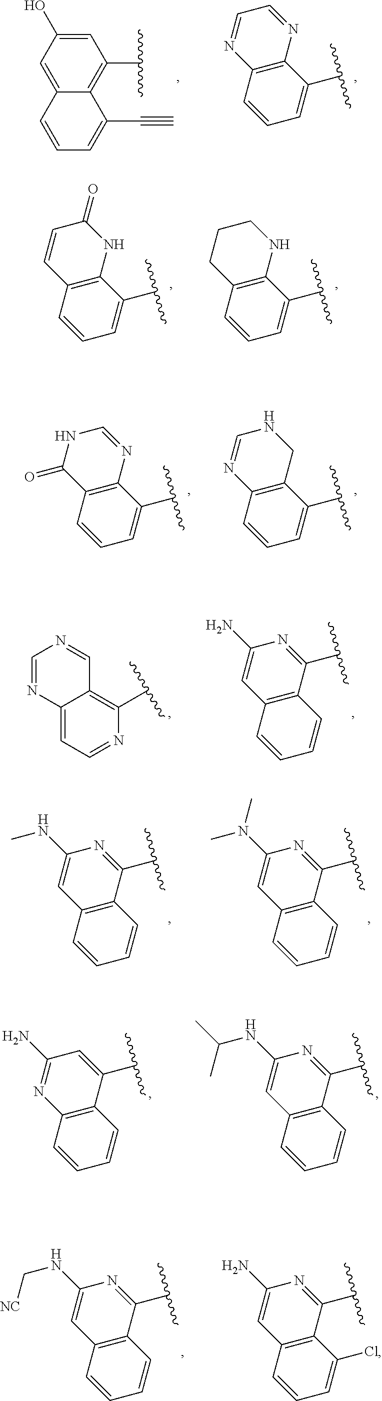

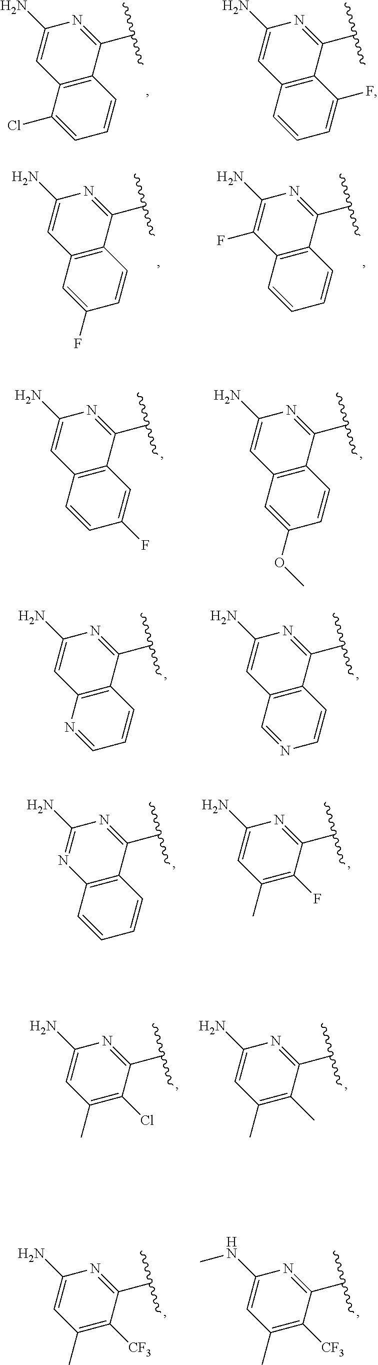

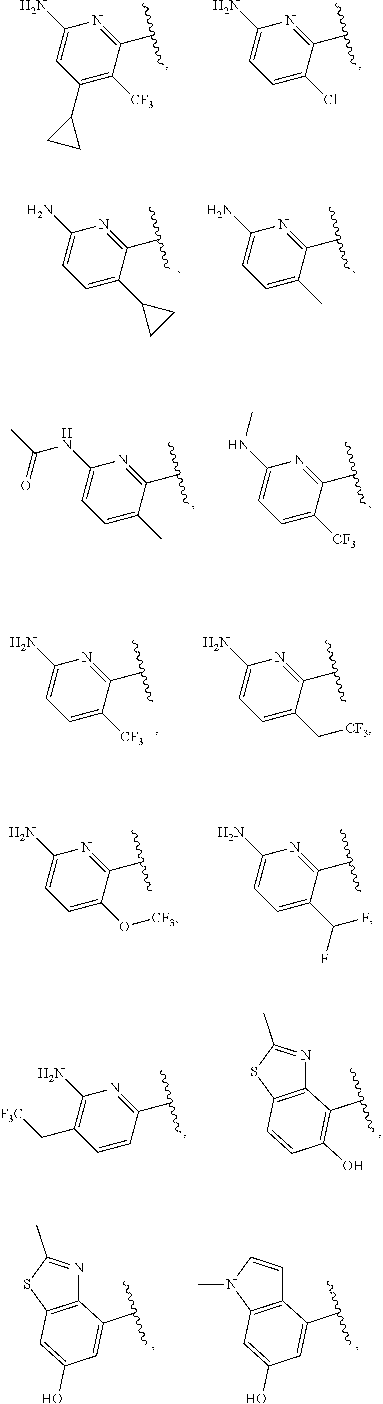

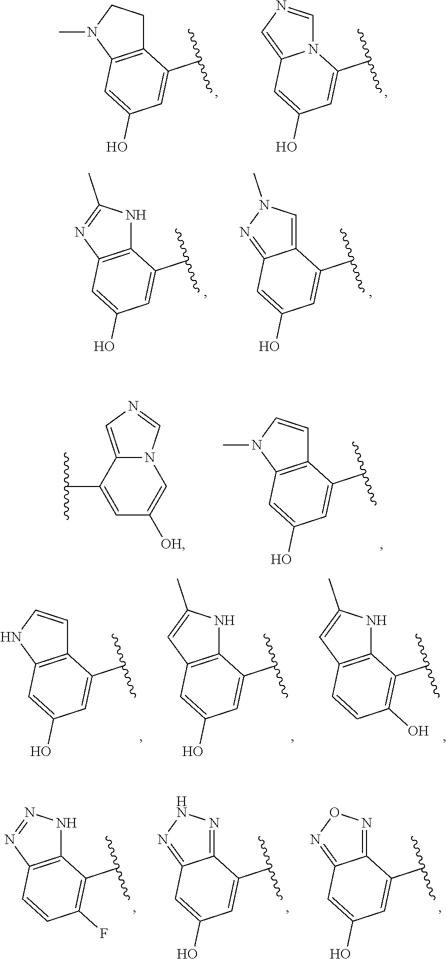

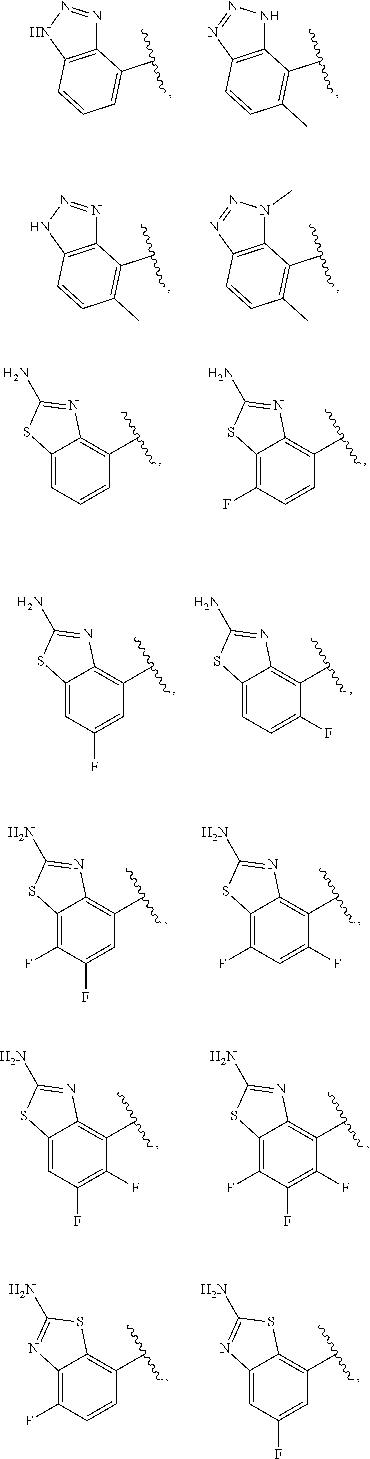

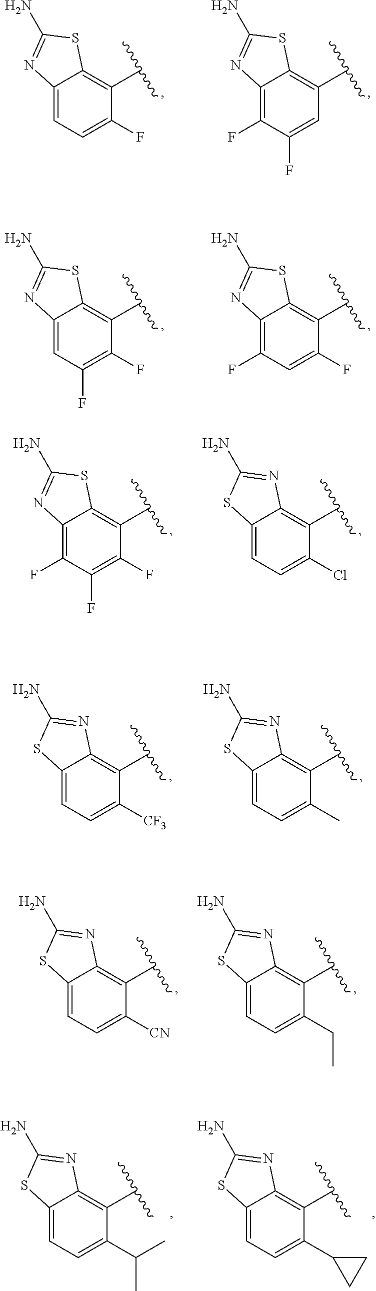

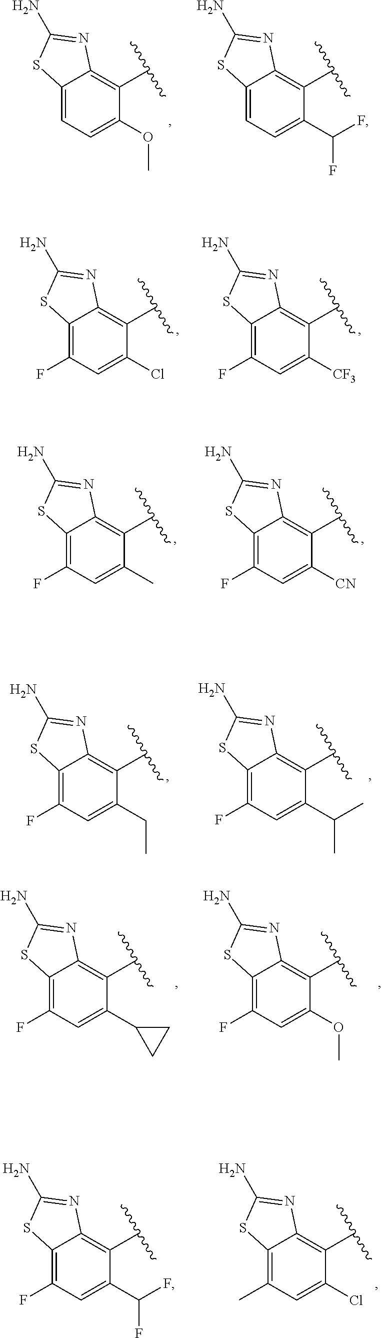

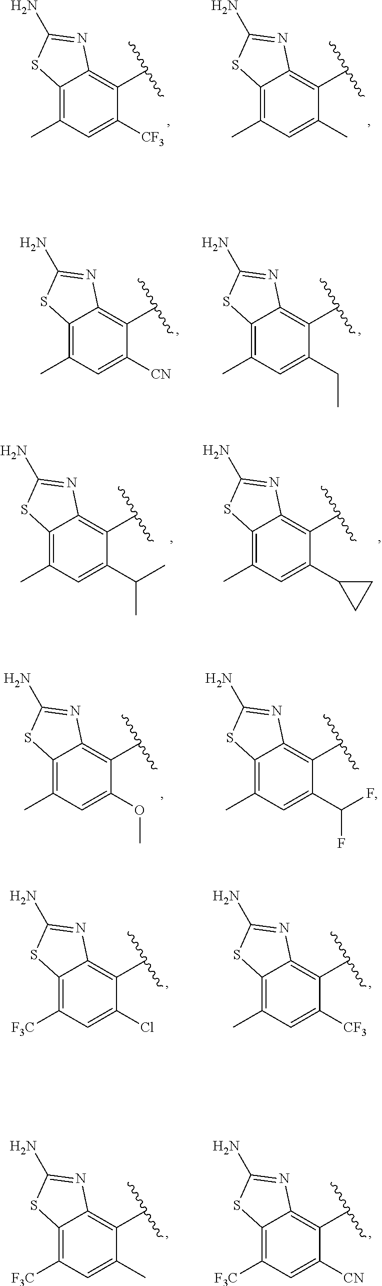

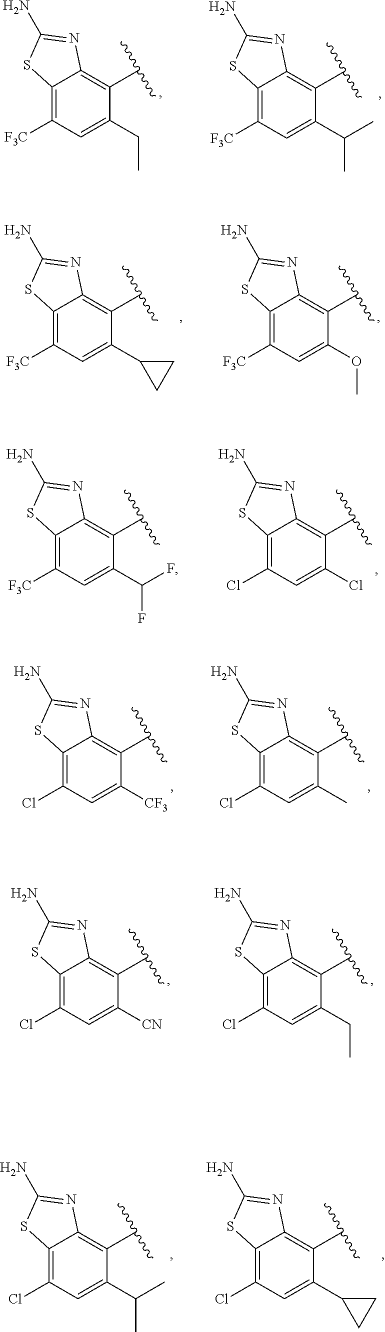

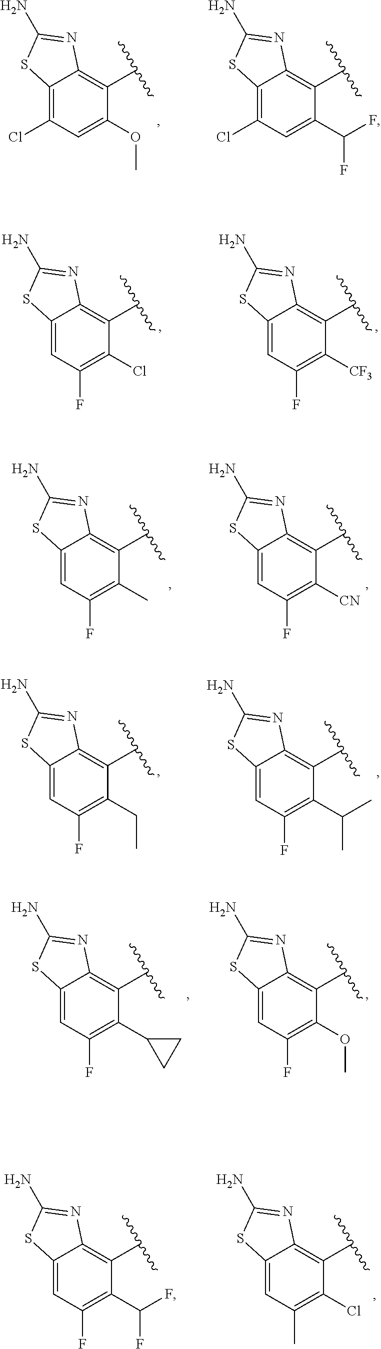

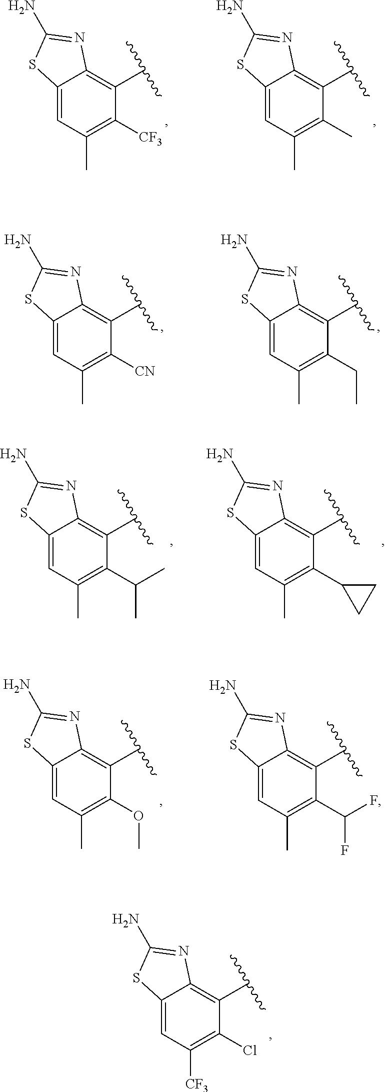

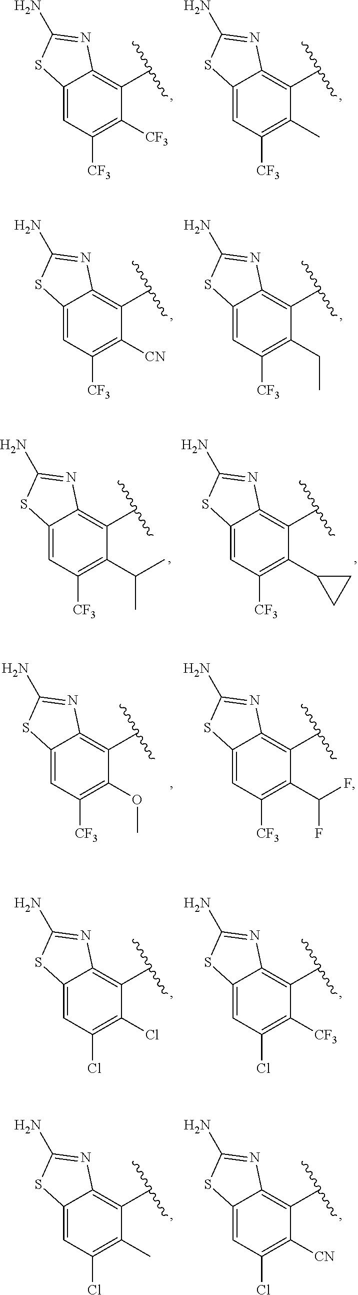

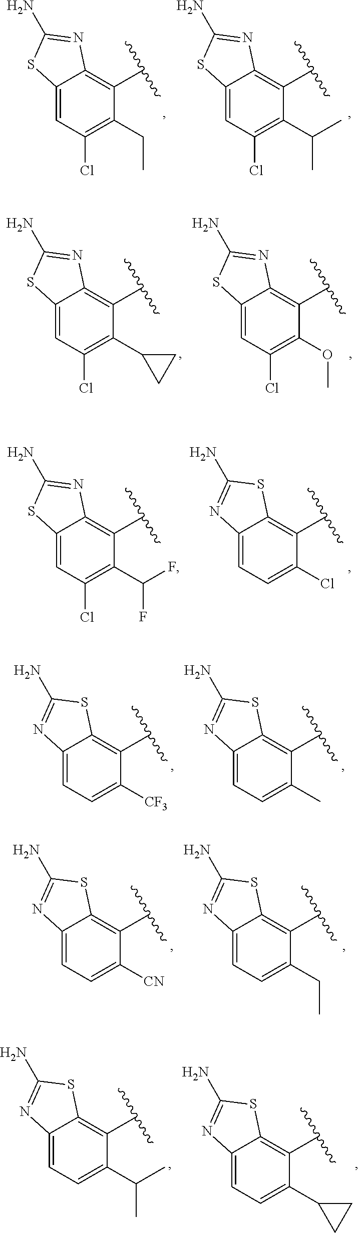

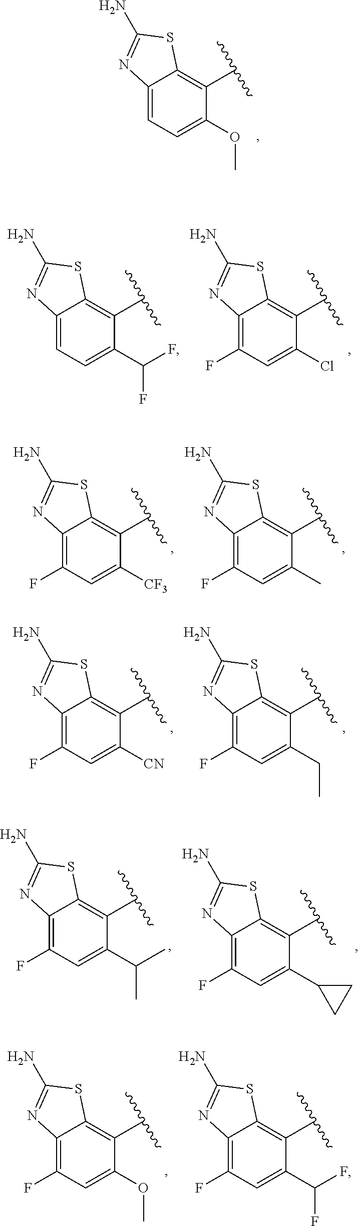

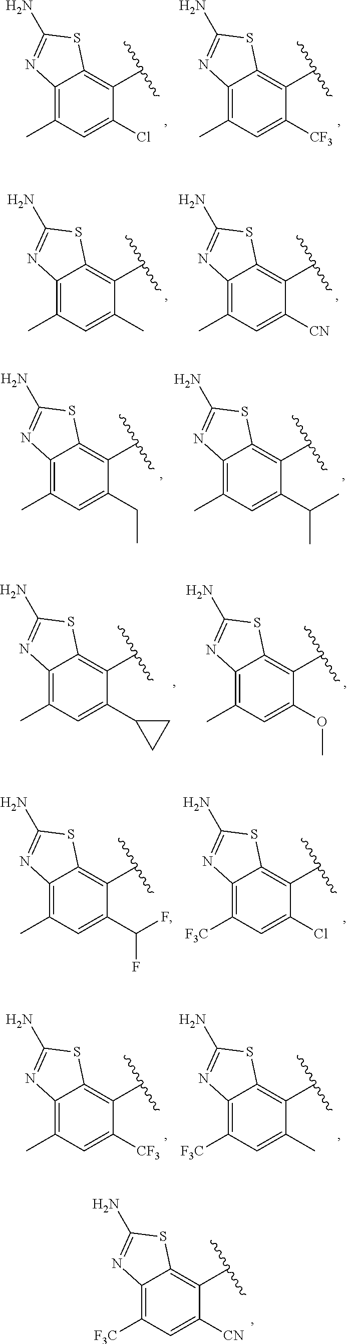

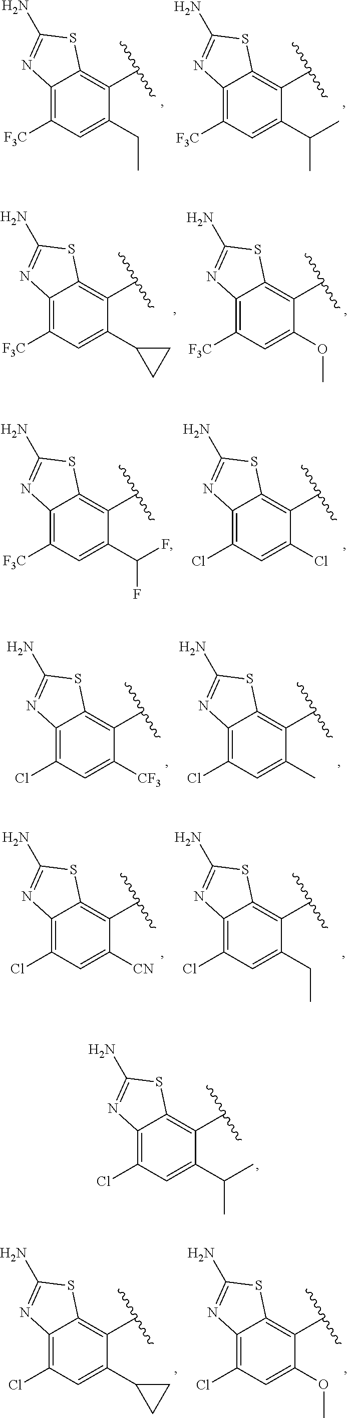

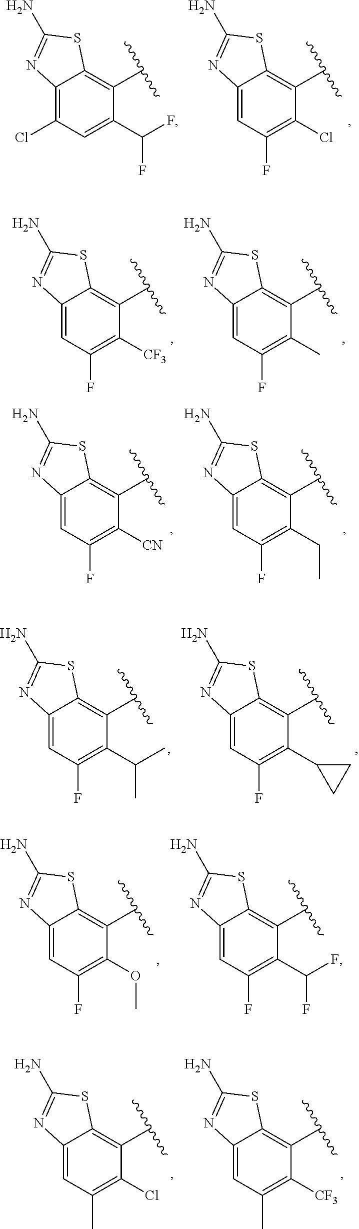

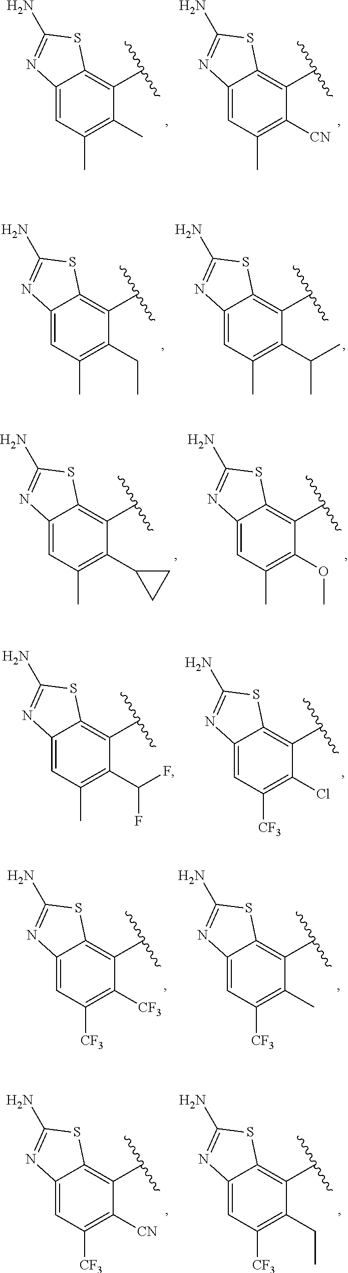

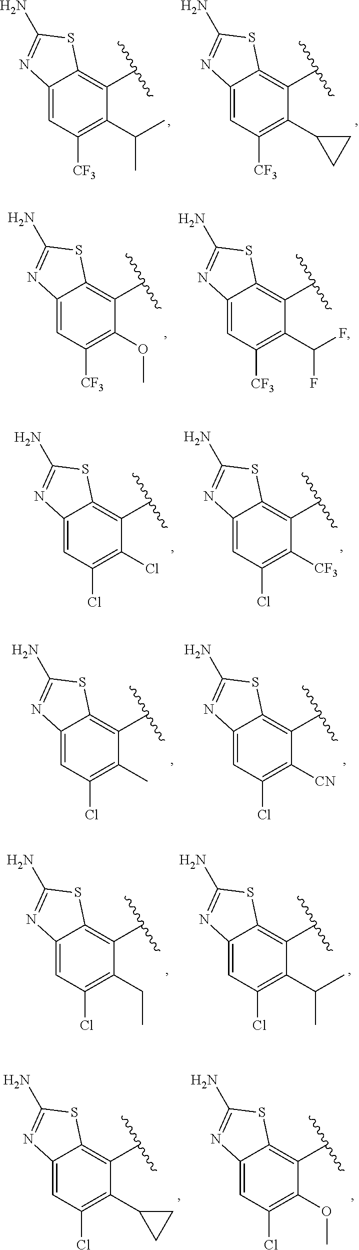

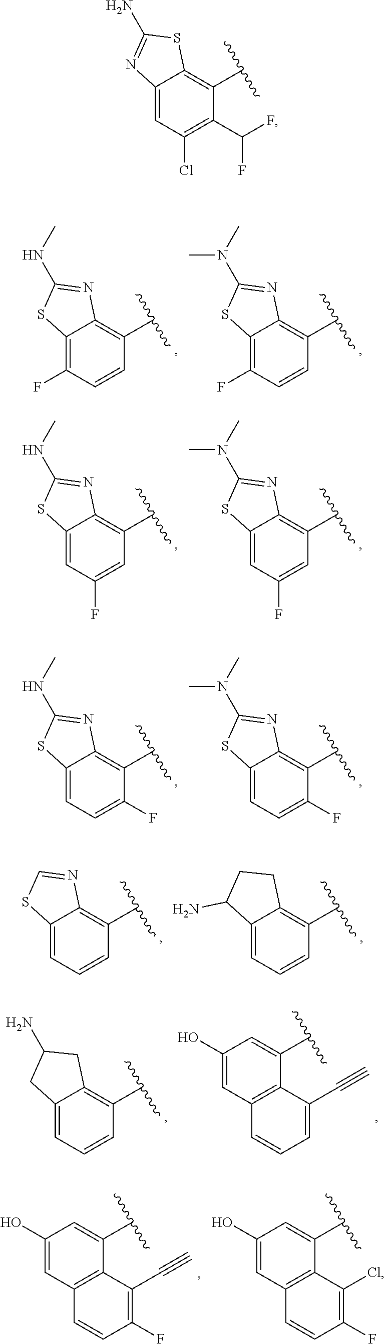

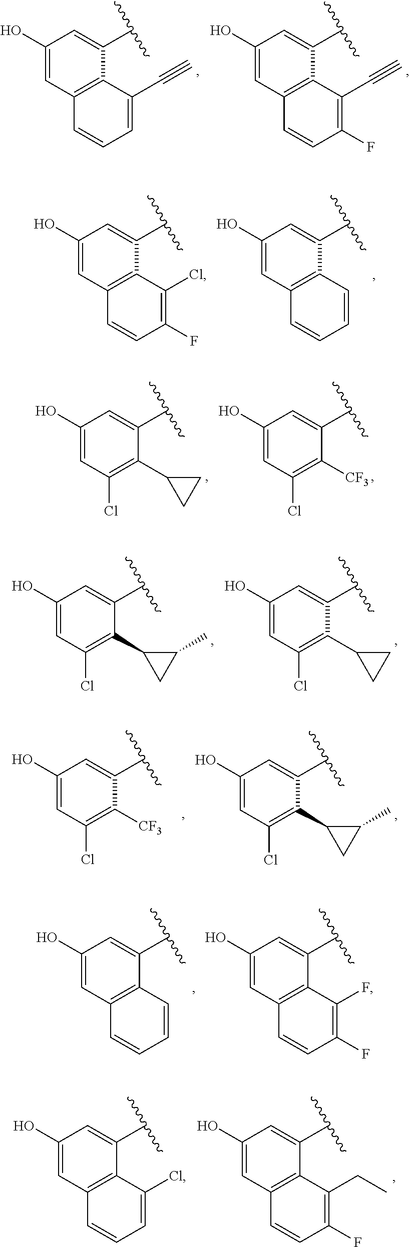

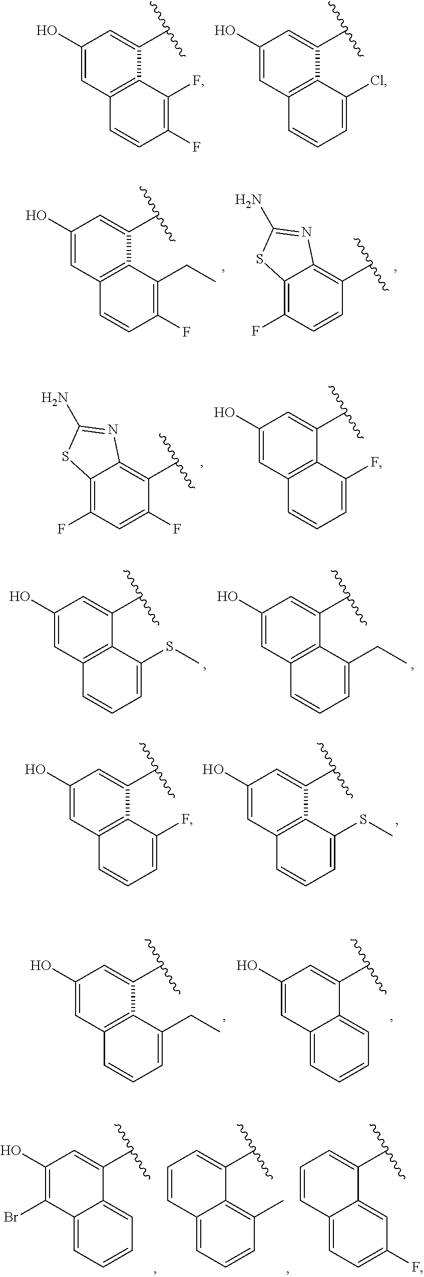

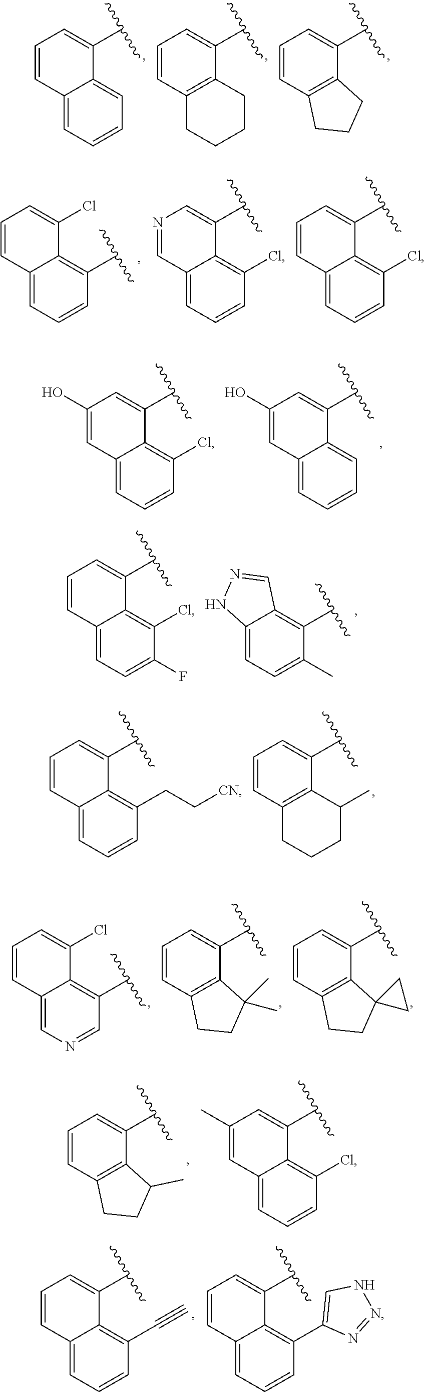

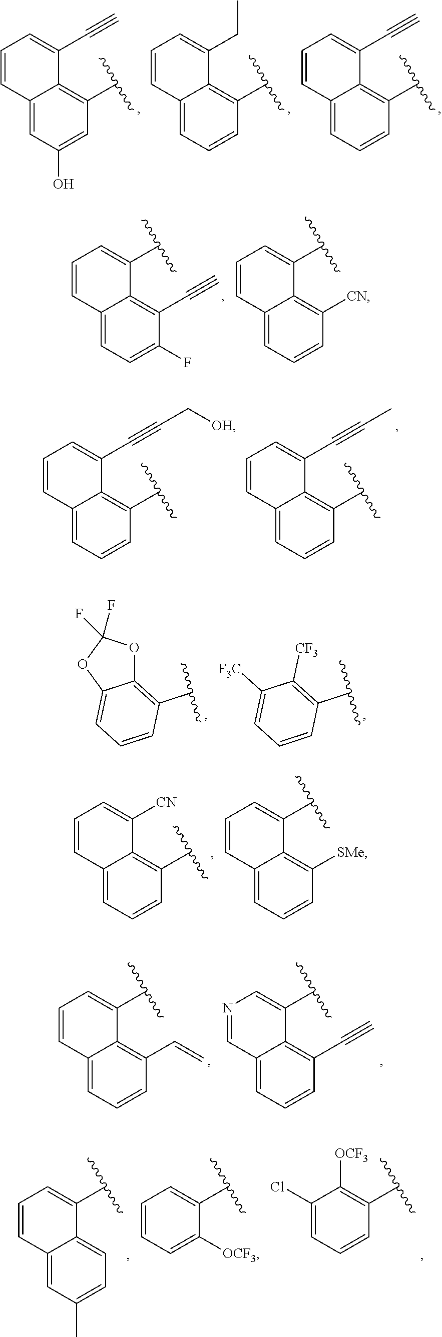

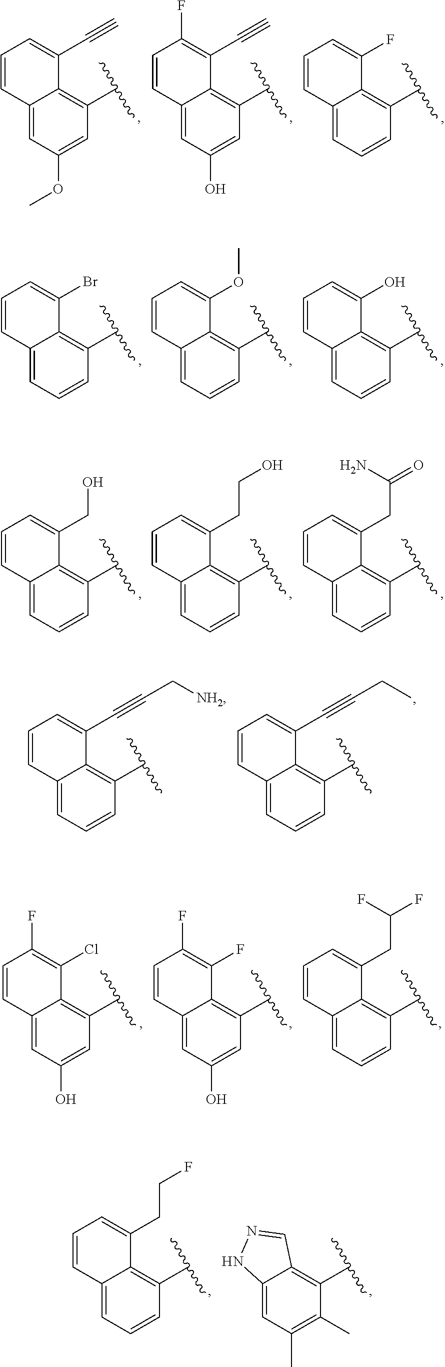

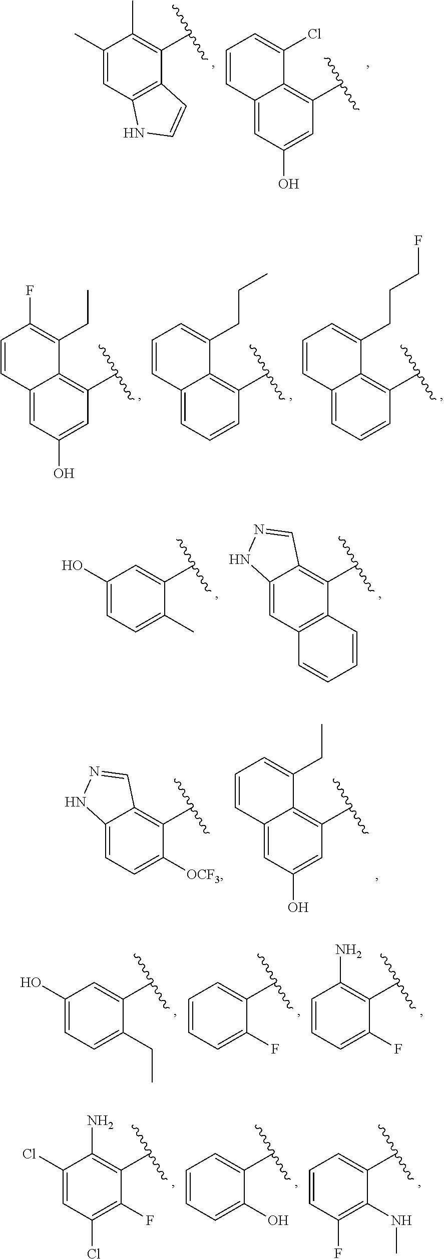

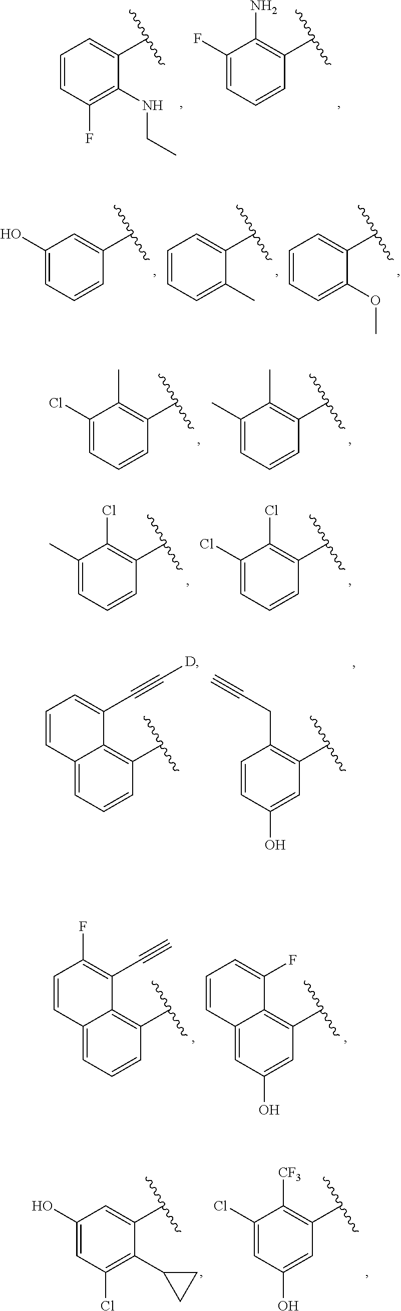

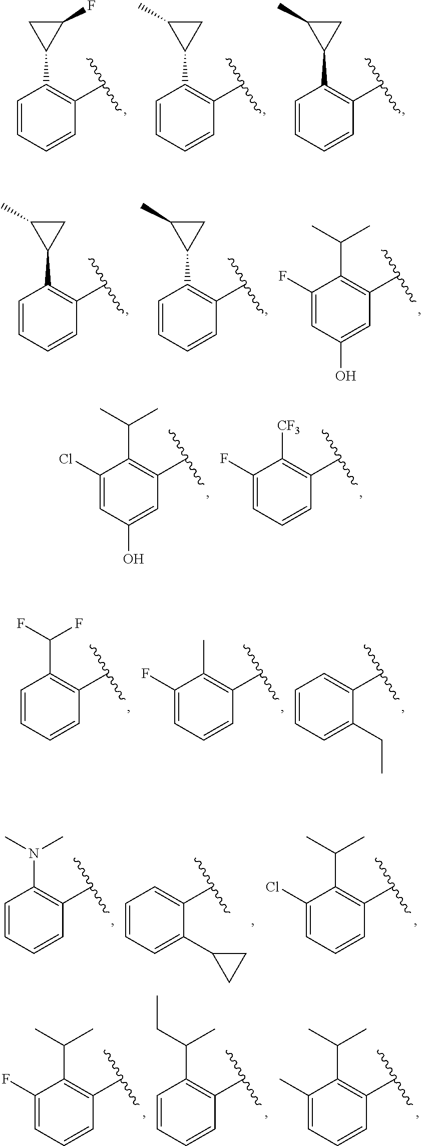

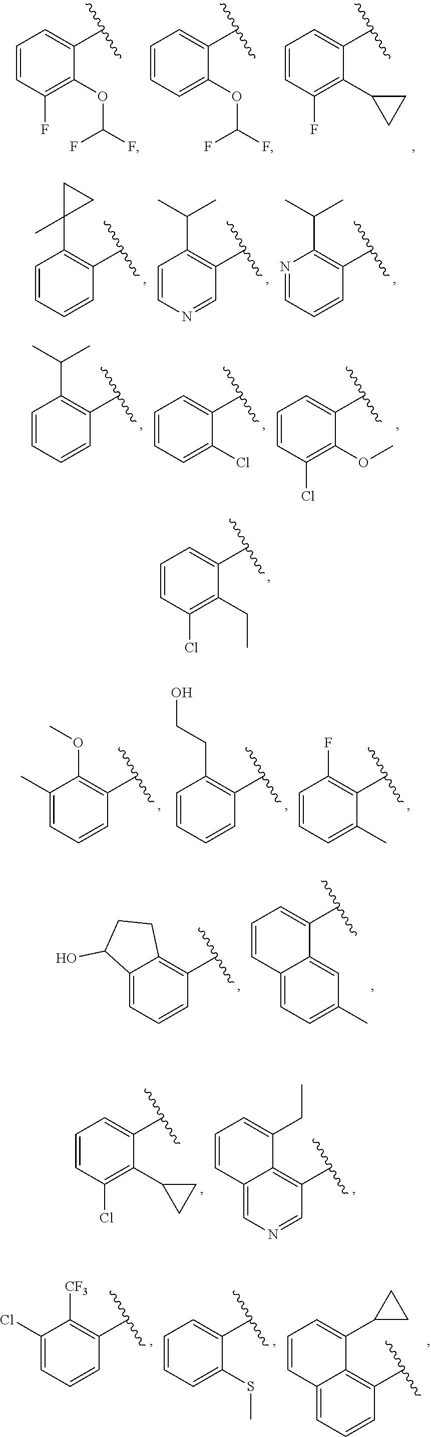

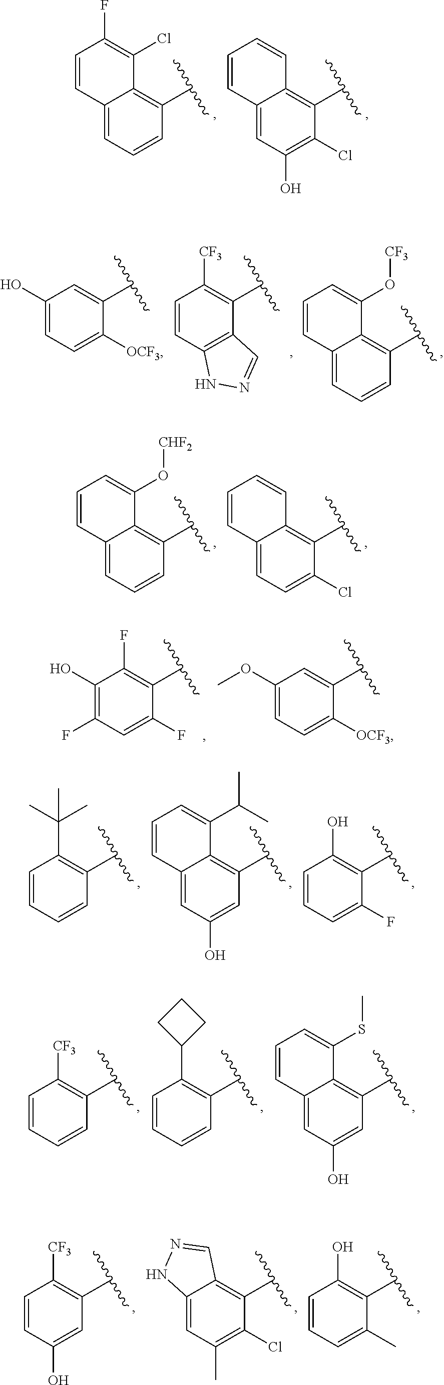

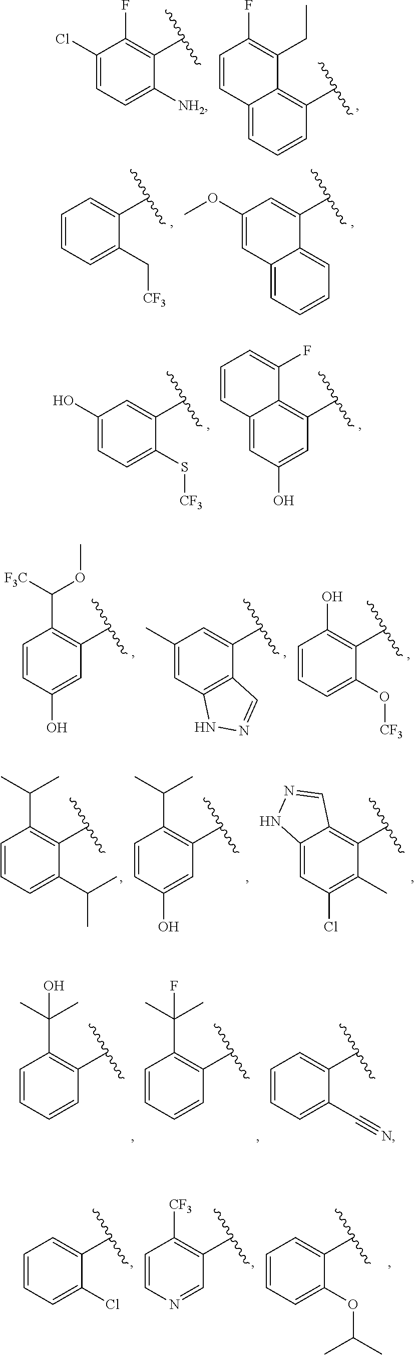

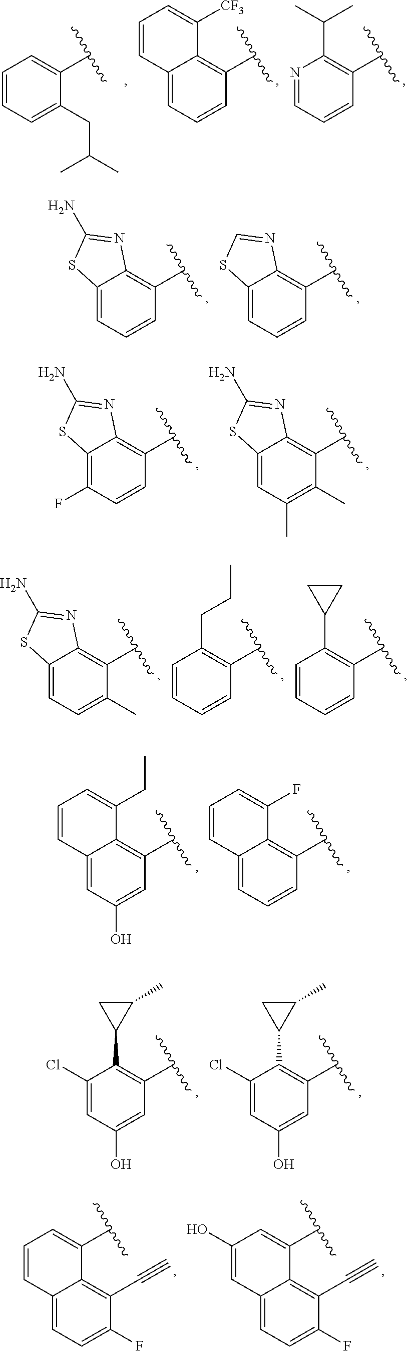

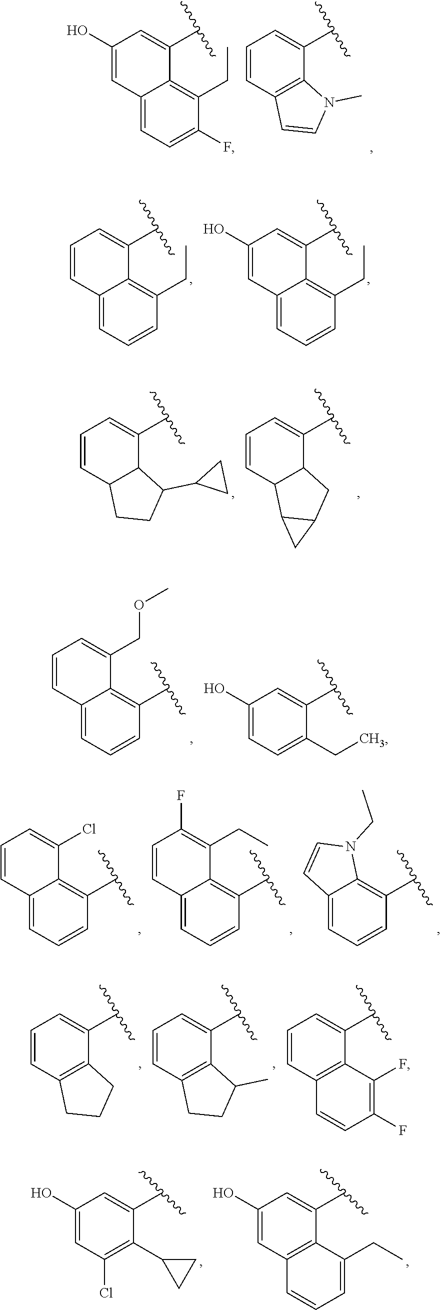

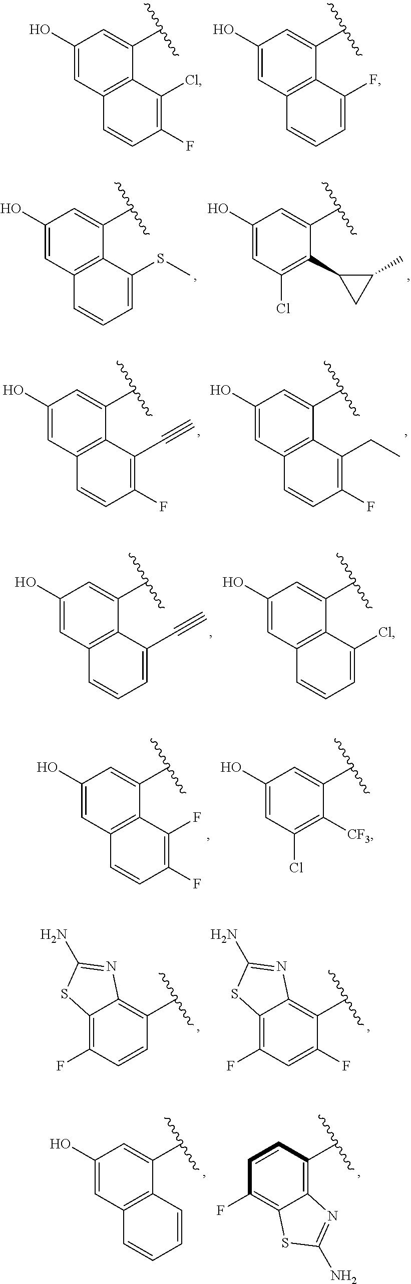

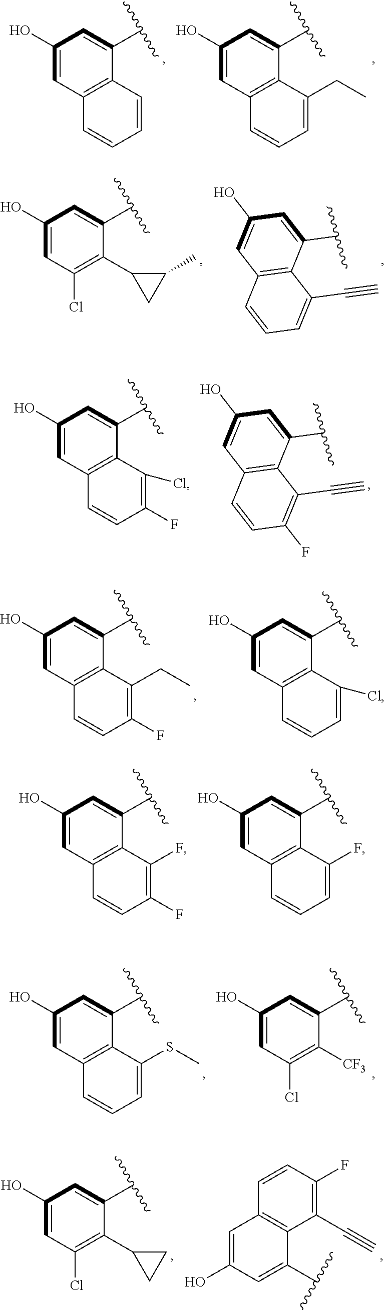

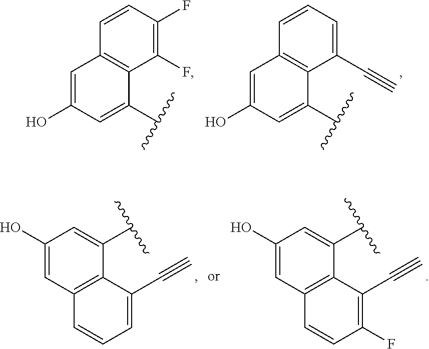 [SMILES: *c1cc(O)cc2ccc(F)c(F)c12 ; *c1cc(O)cc2cccc(C#C)c12 ; *c1cc(O)cc2cccc(C#C)c12 ; *c1cc(O)cc2ccc(F)c(C#C)c12]

[96]. The compound according to any one of [1] to [95], wherein, R₃ is selected from

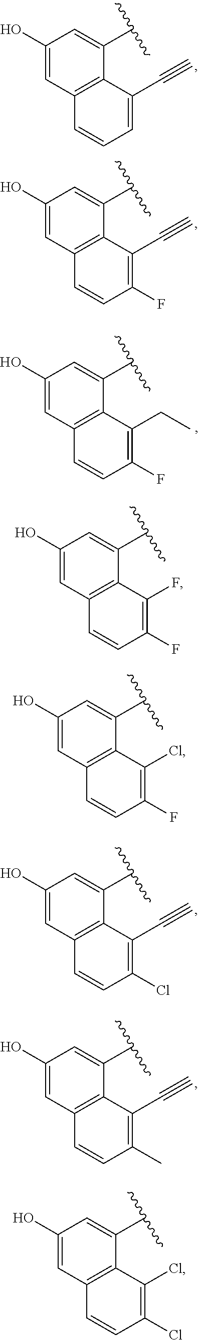

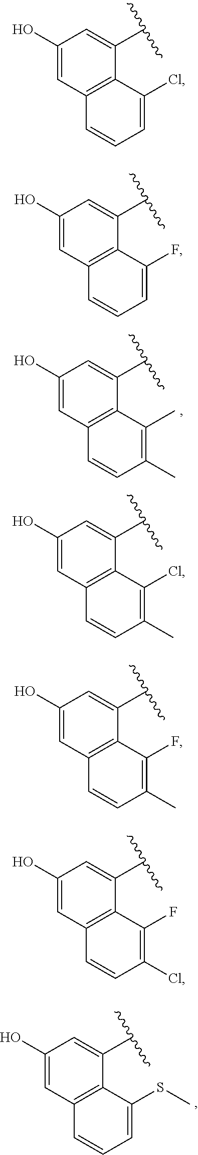

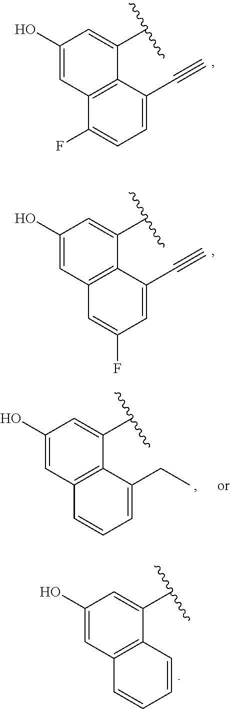 [SMILES: *c1cc(O)cc2c(F)ccc(C#C)c12 ; *c1cc(O)cc2cc(F)cc(C#C)c12 ; *c1cc(O)cc2cccc(CC)c12 ; *c1cc(O)cc2ccccc12]

[97]. The compound according to any one of [1] to [96], wherein R₃ is selected from

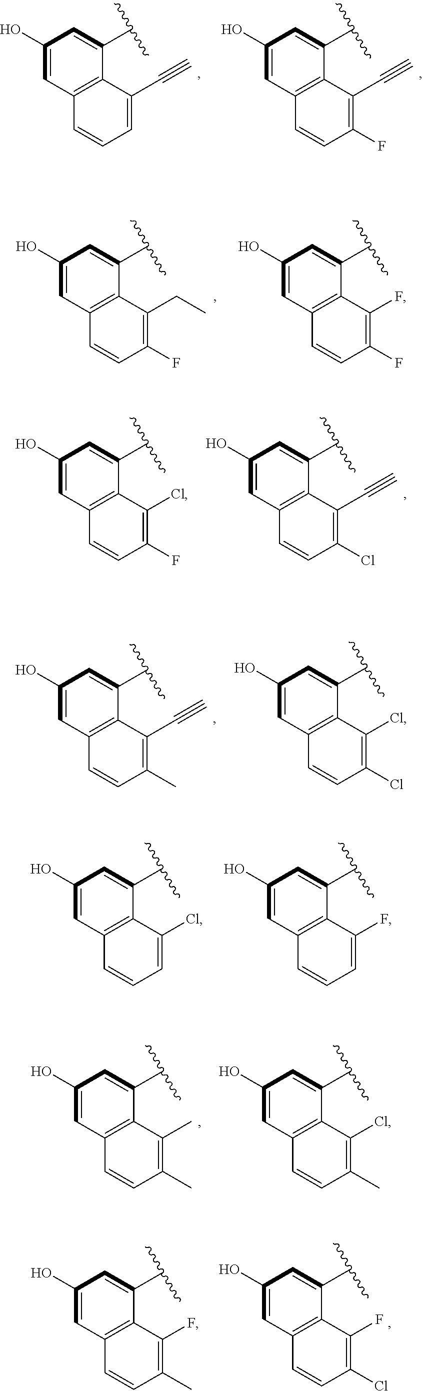

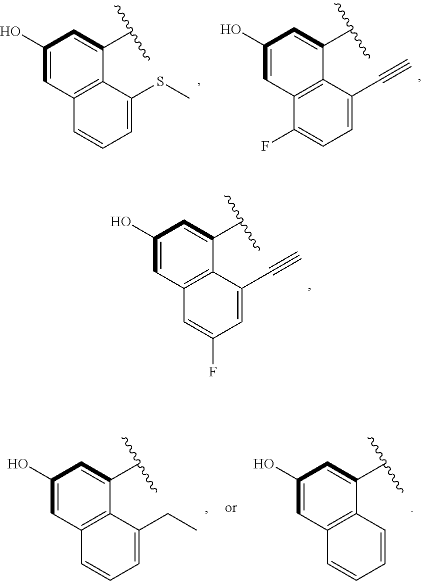

[98]. The compound according to any one of [1] to [97], wherein, R₃ is selected from

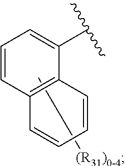

Preferably, the compound of formula (I) is selected from any one of the following formulas:

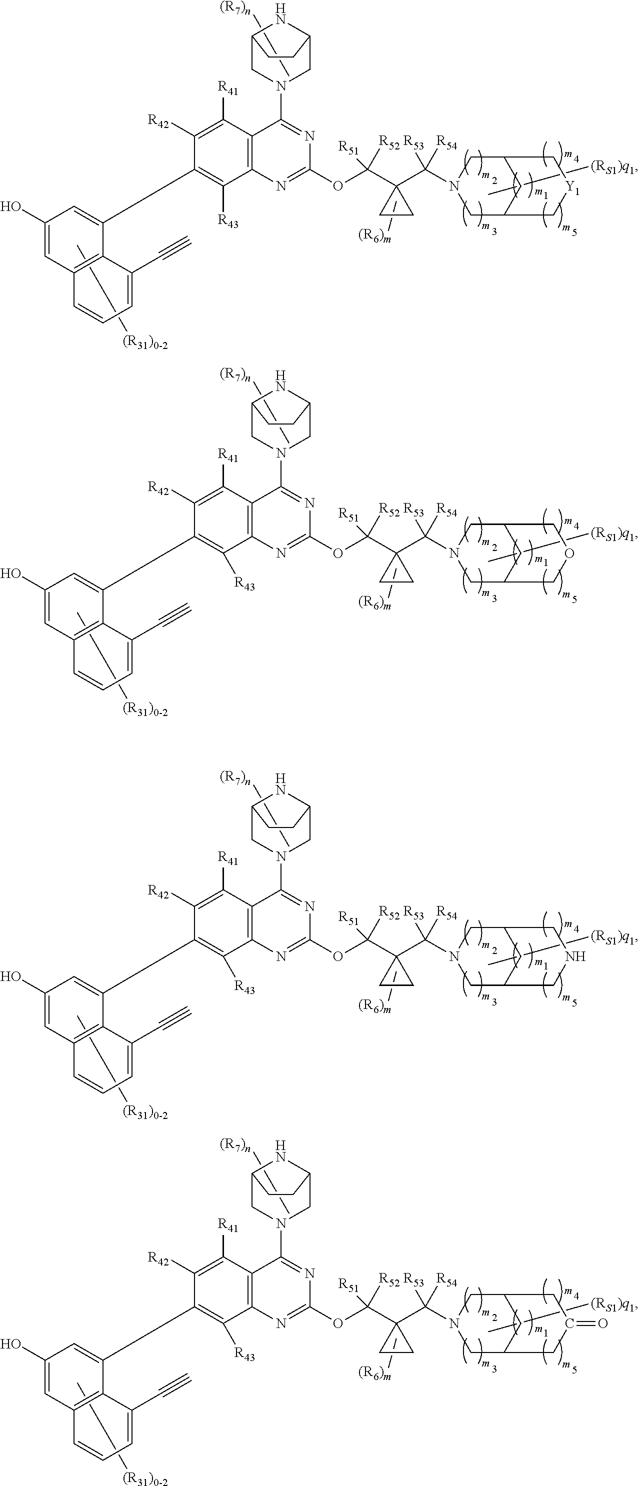

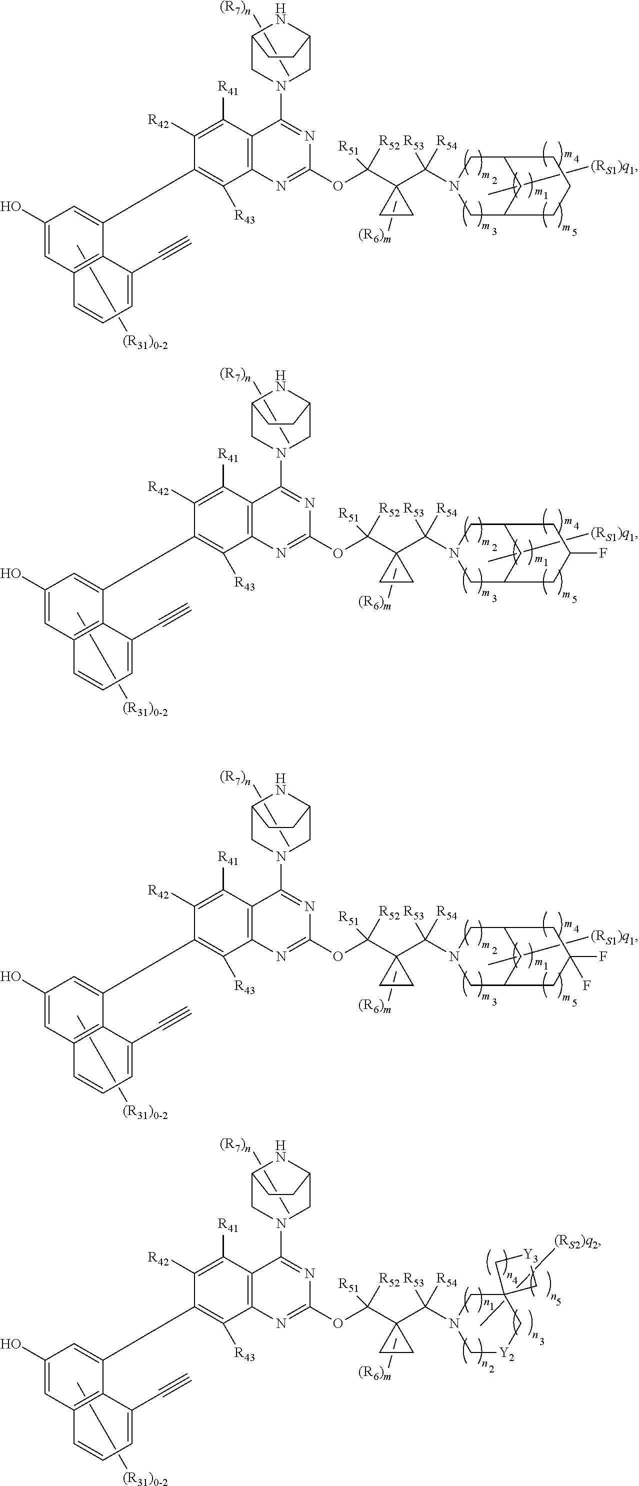

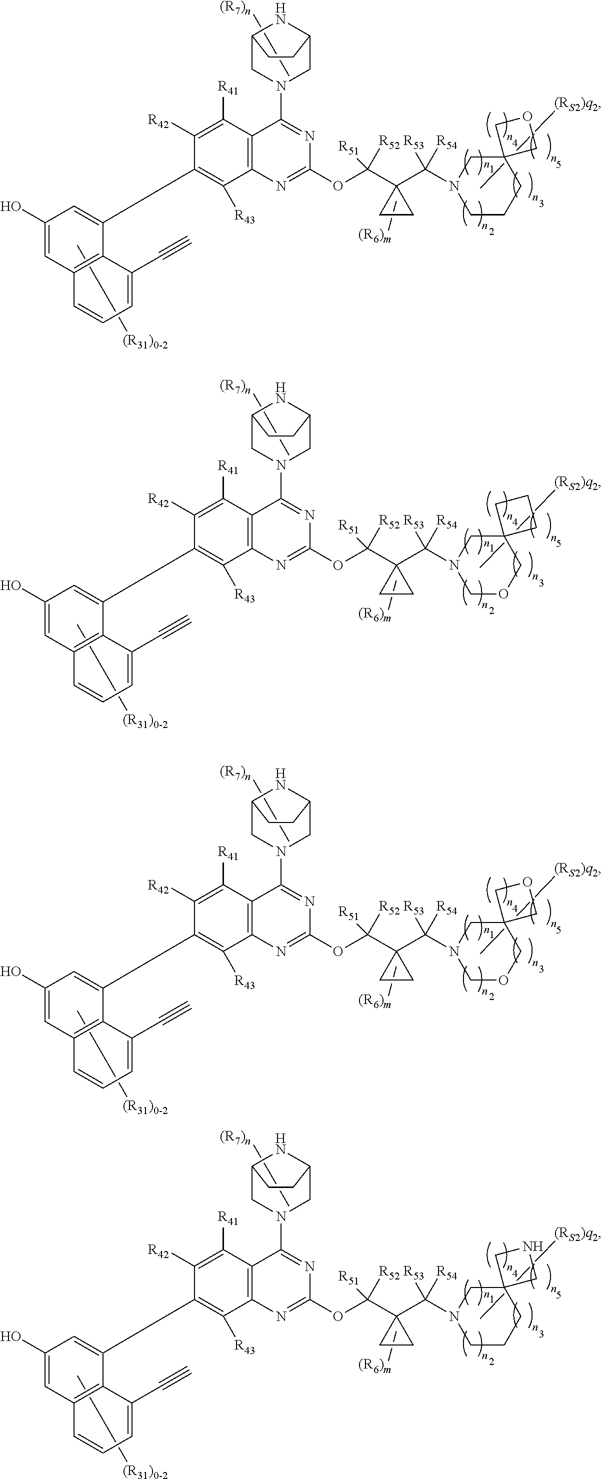

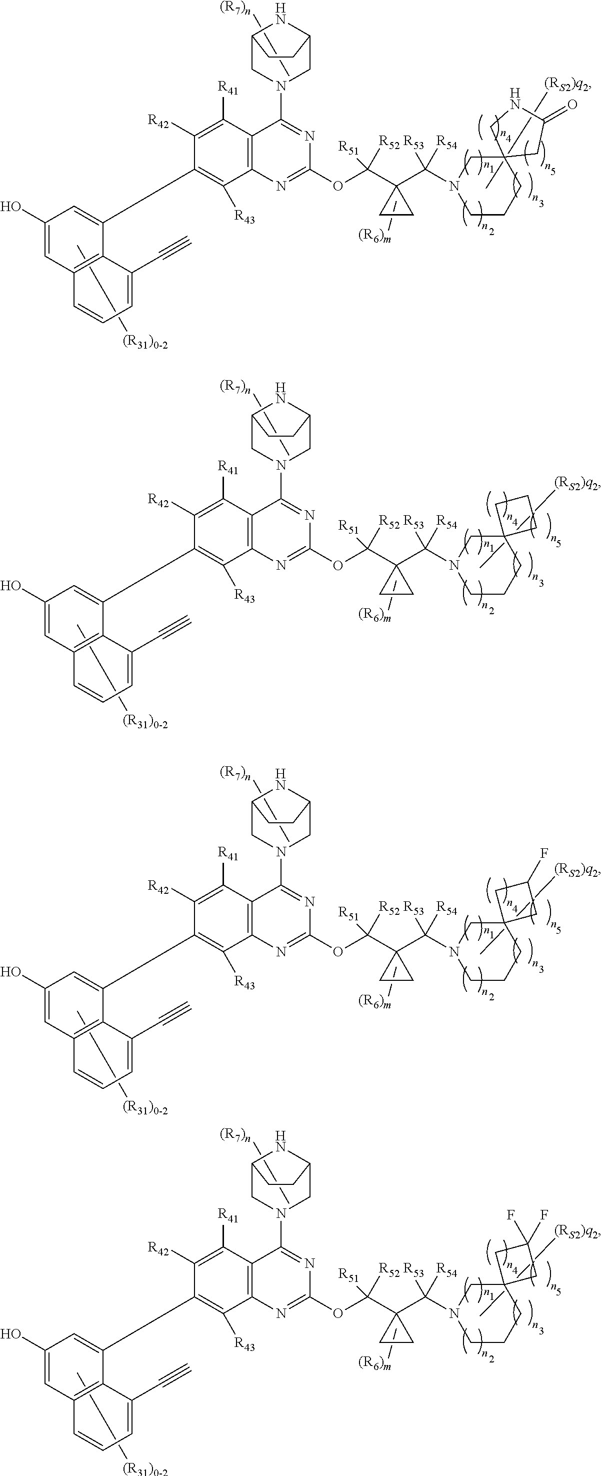

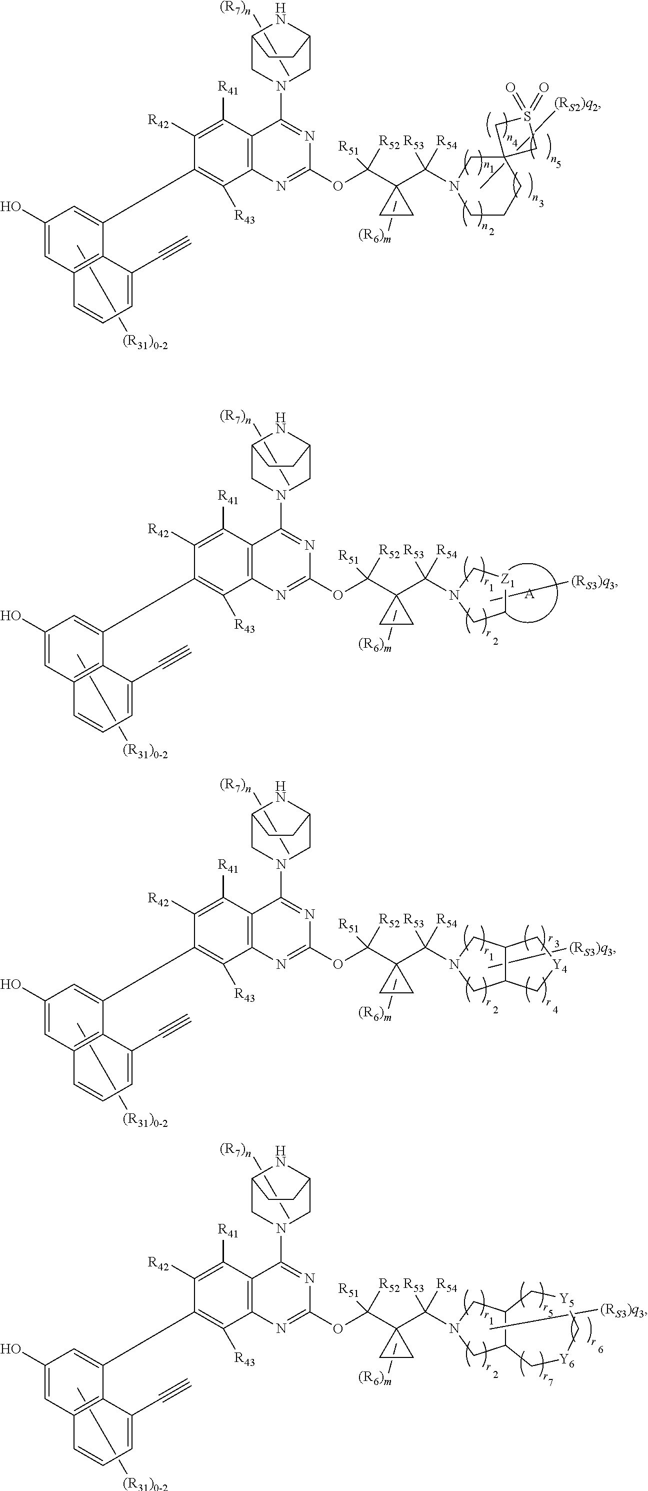

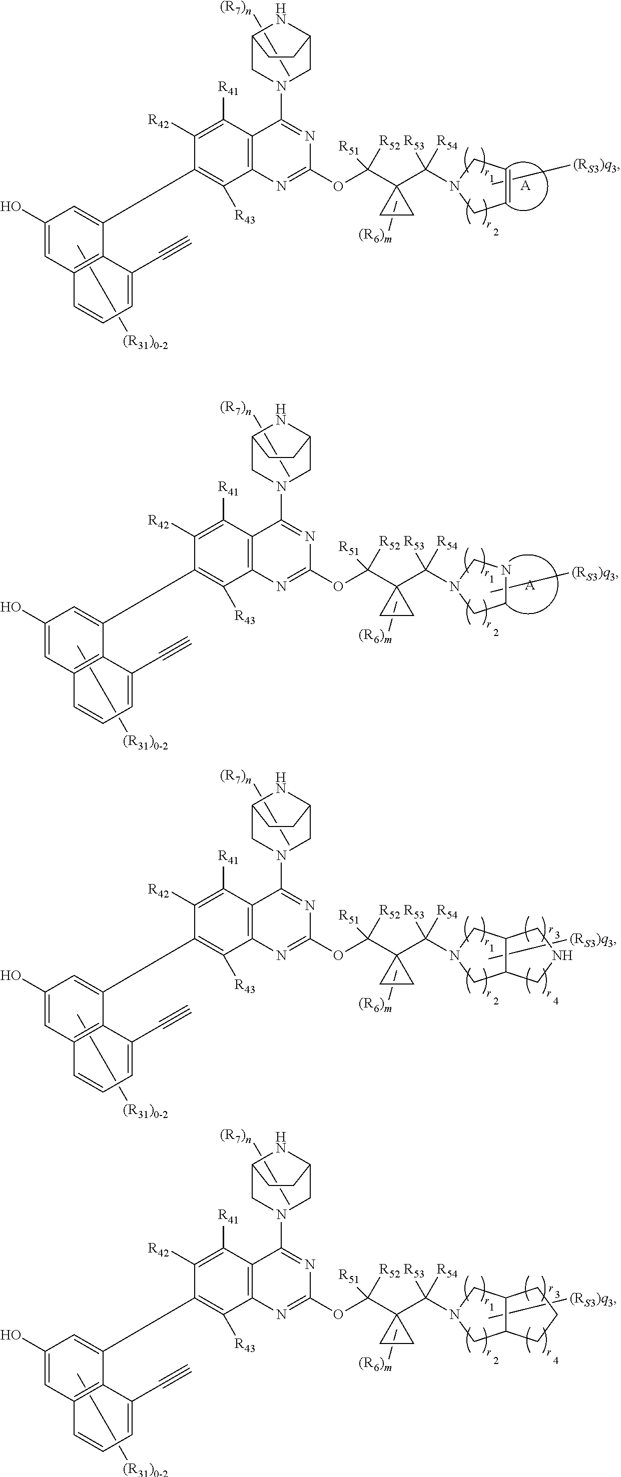

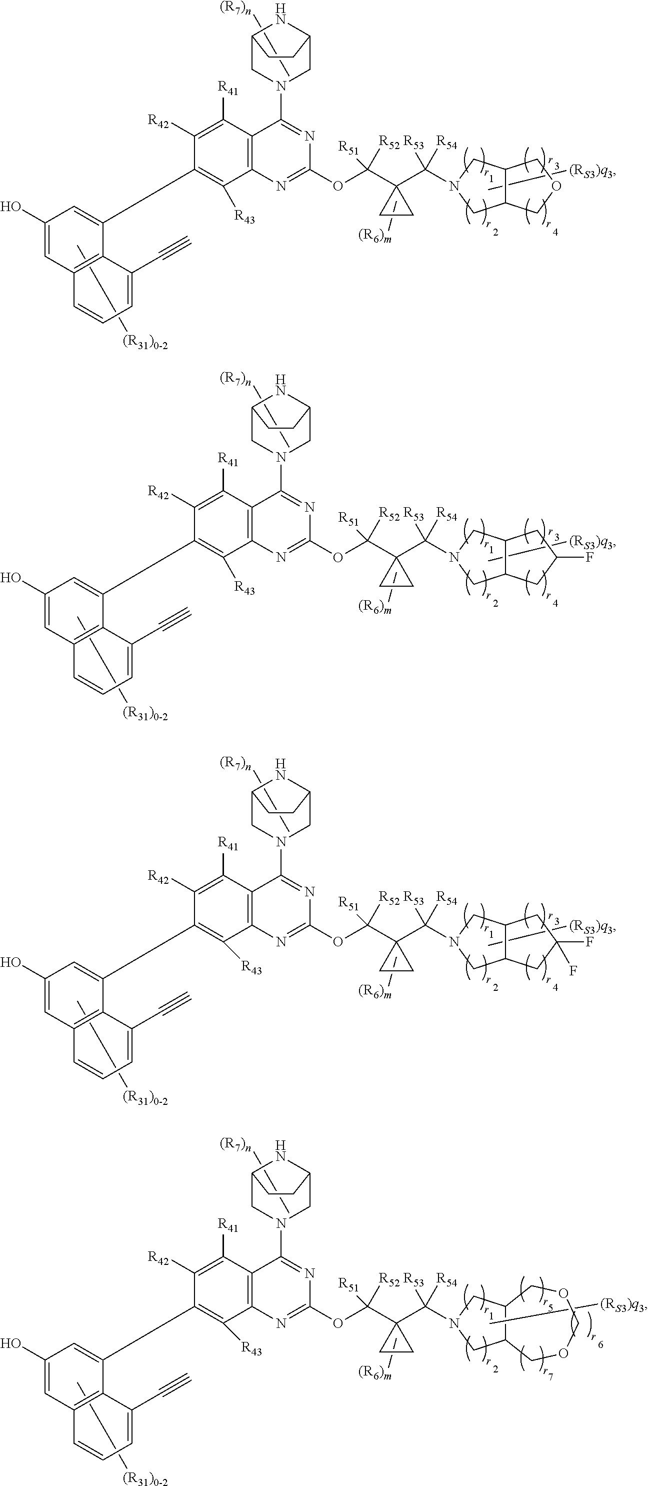

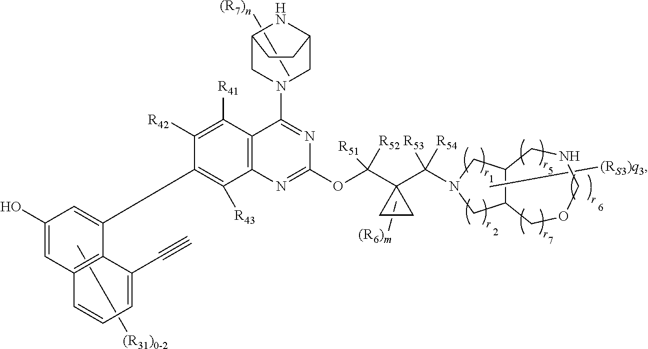

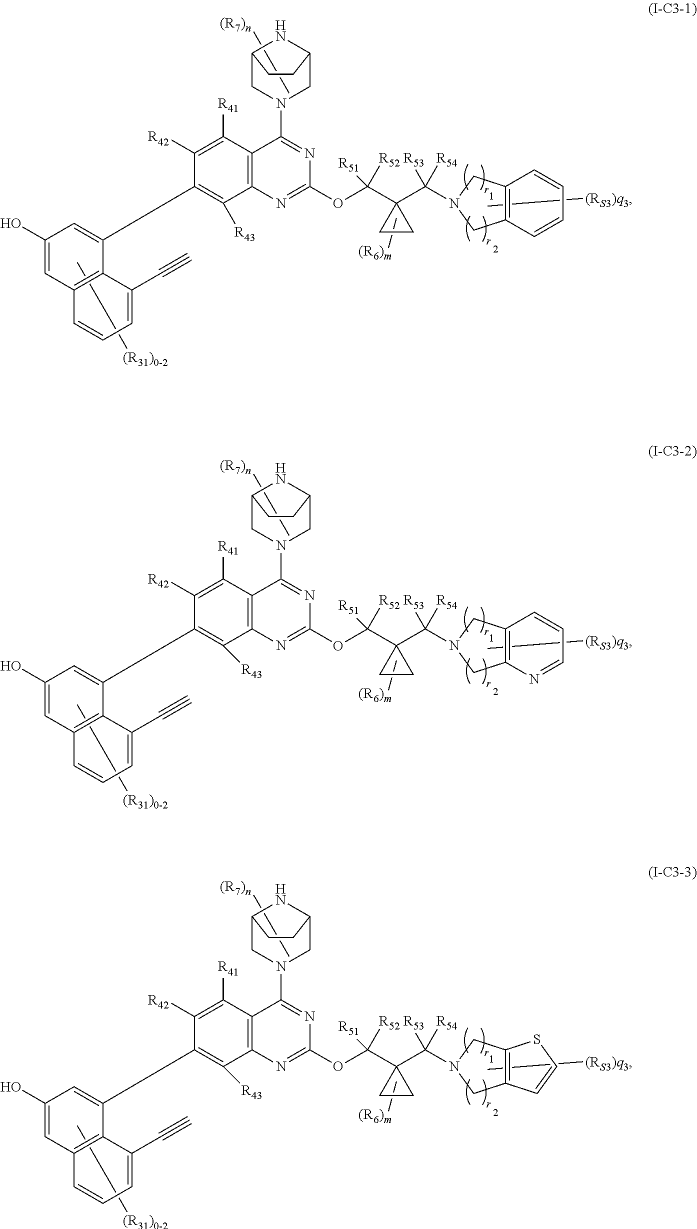

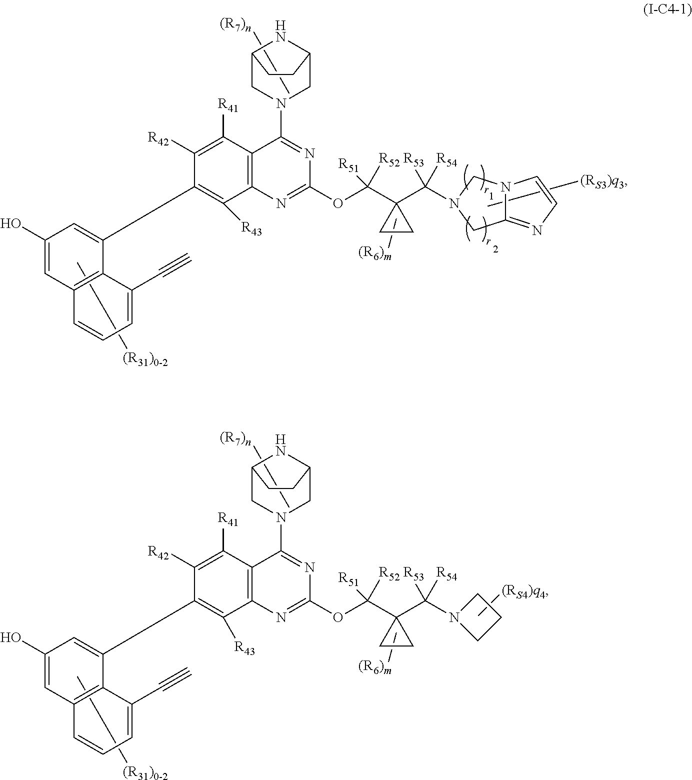

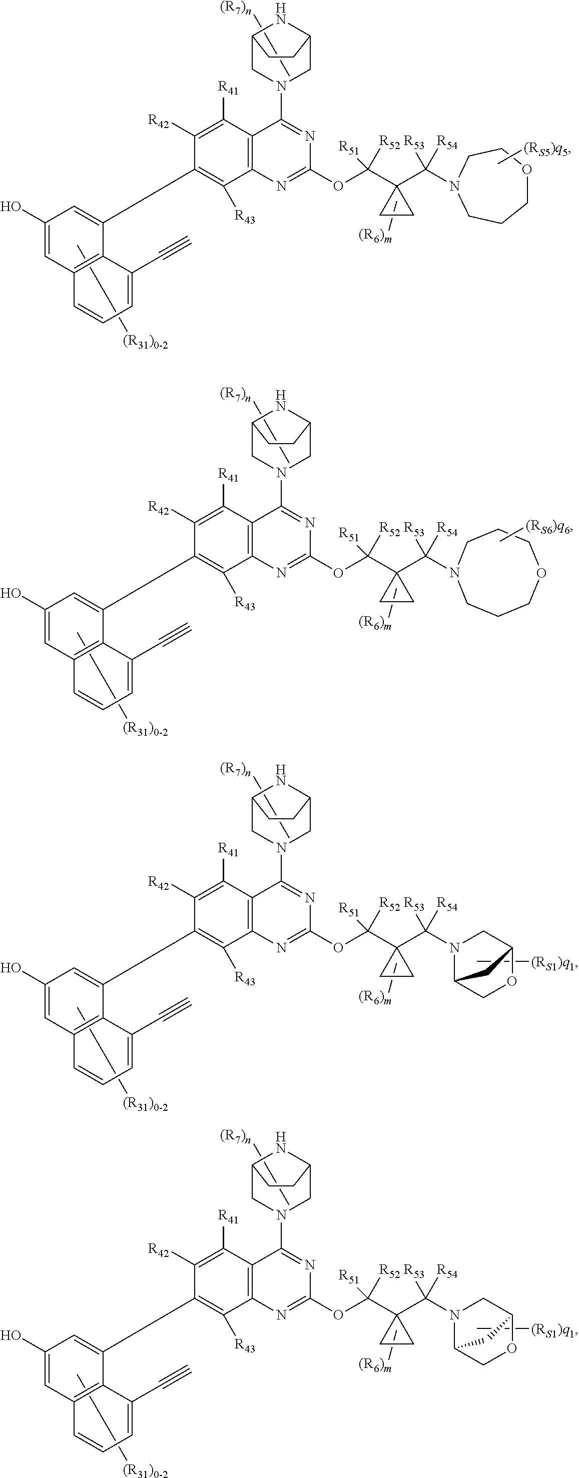

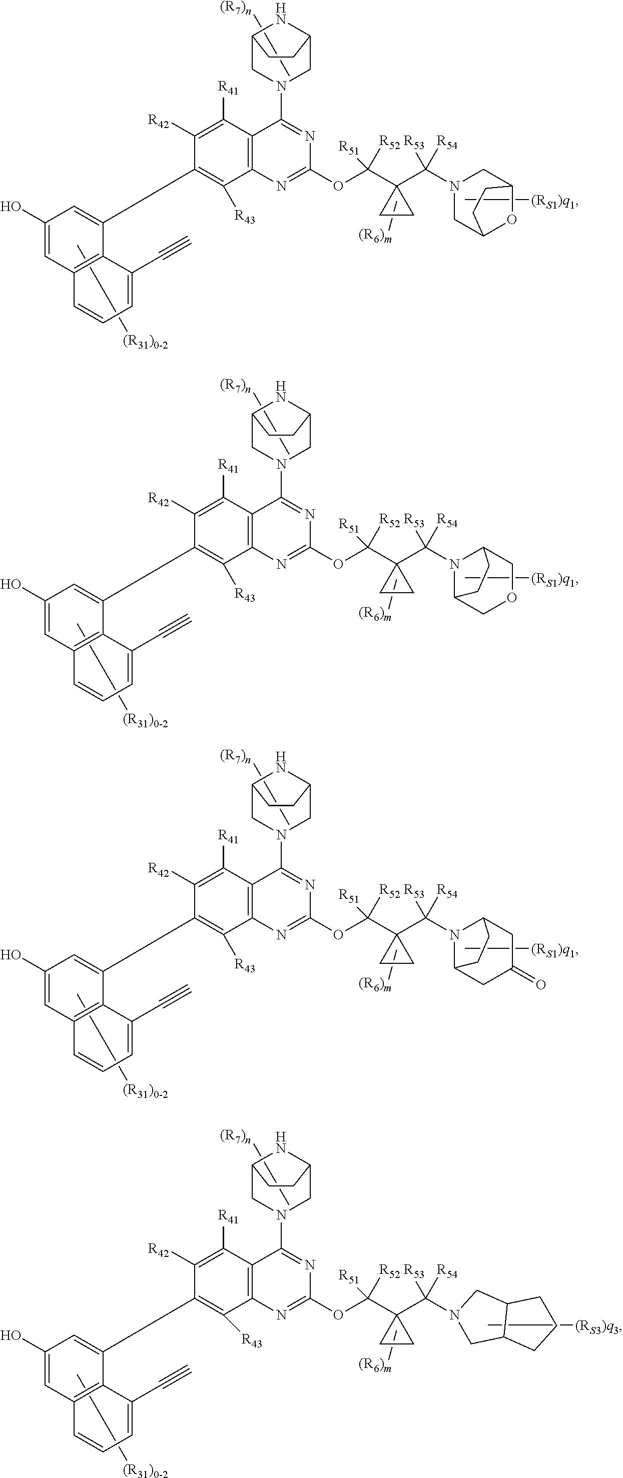

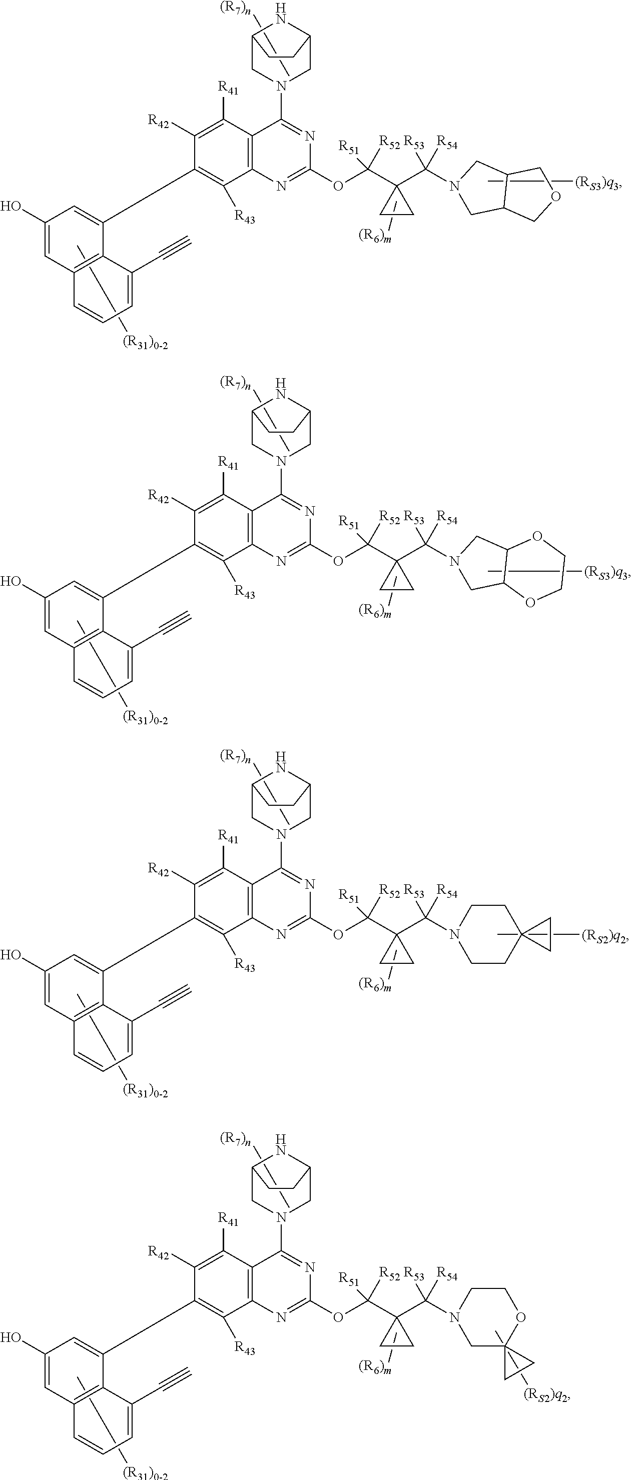

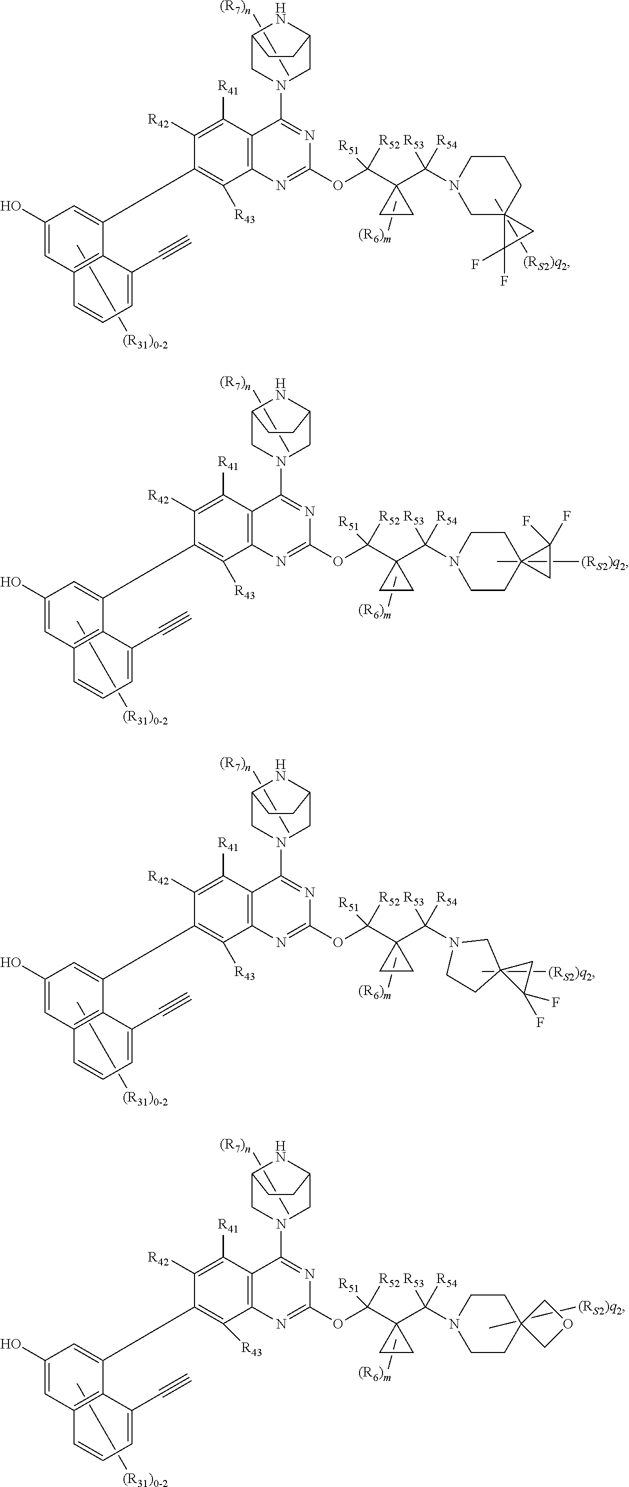

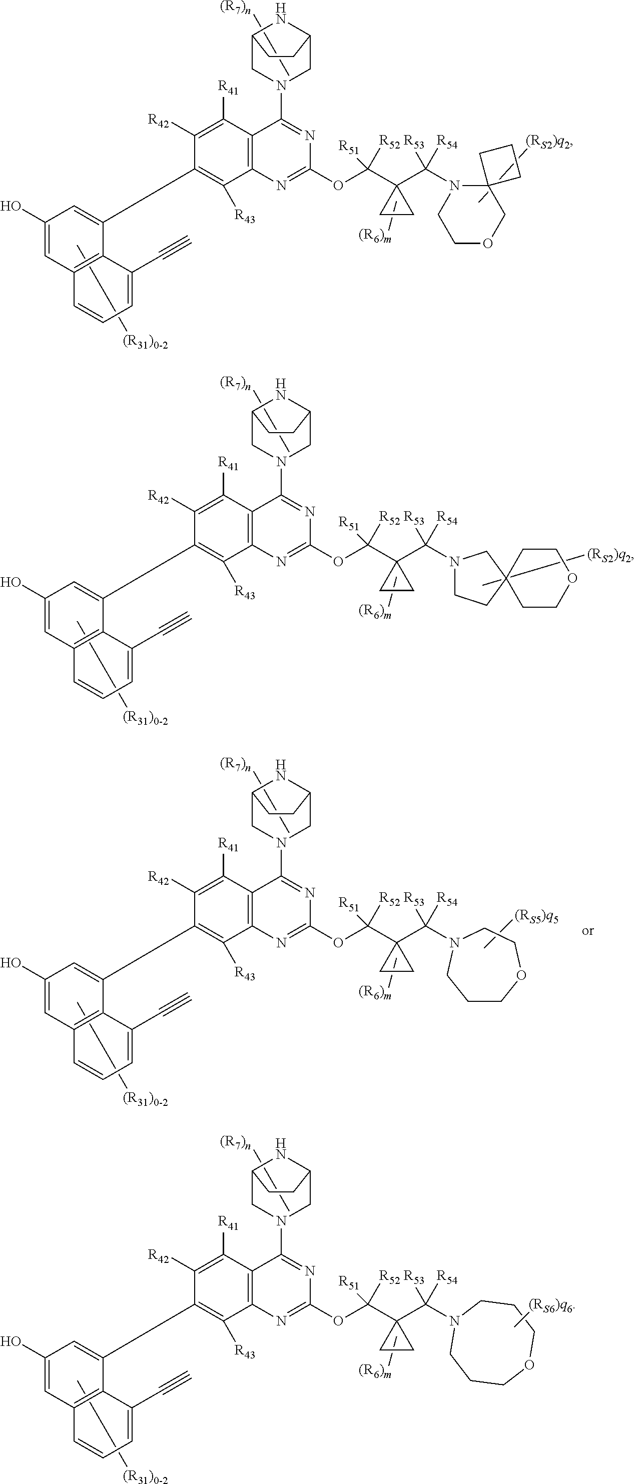

[99]. The compound according to any one of [1] to [98], wherein, R₃ is selected from

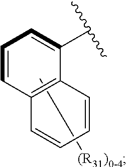

Preferably, the compound of formula (I) is selected from any one of the following formulas:

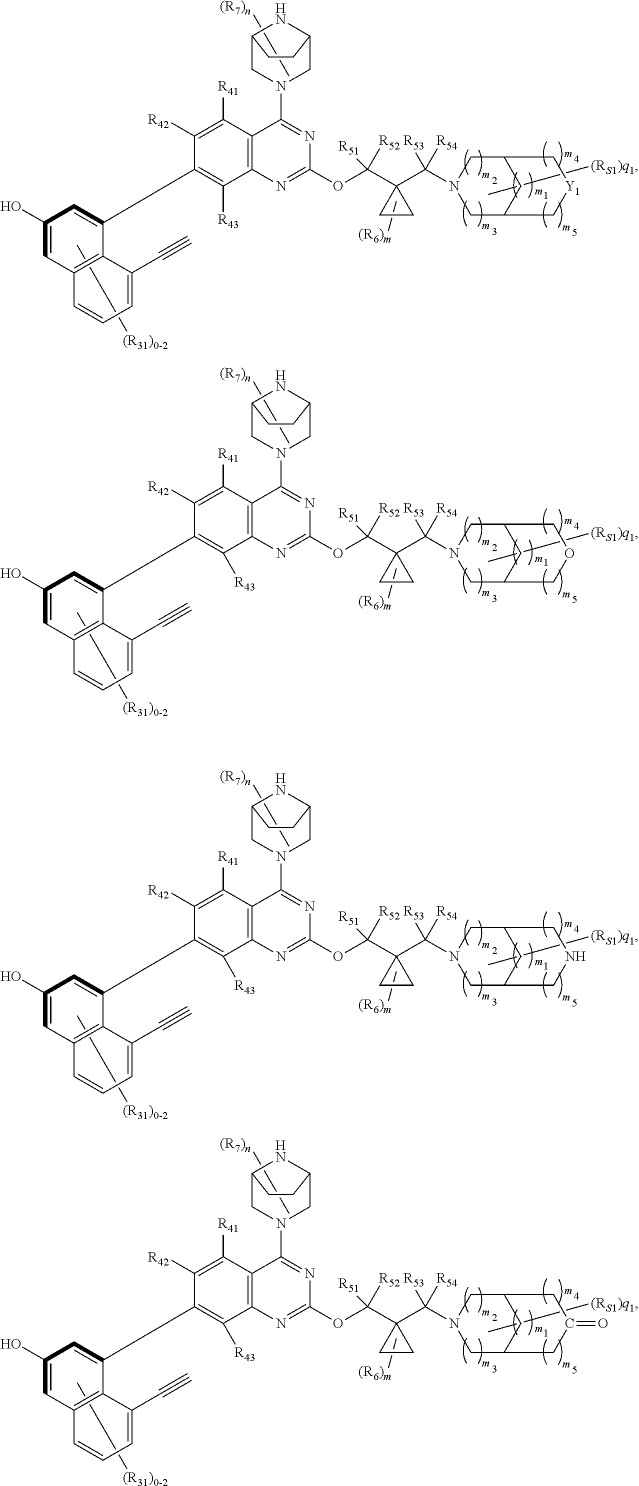

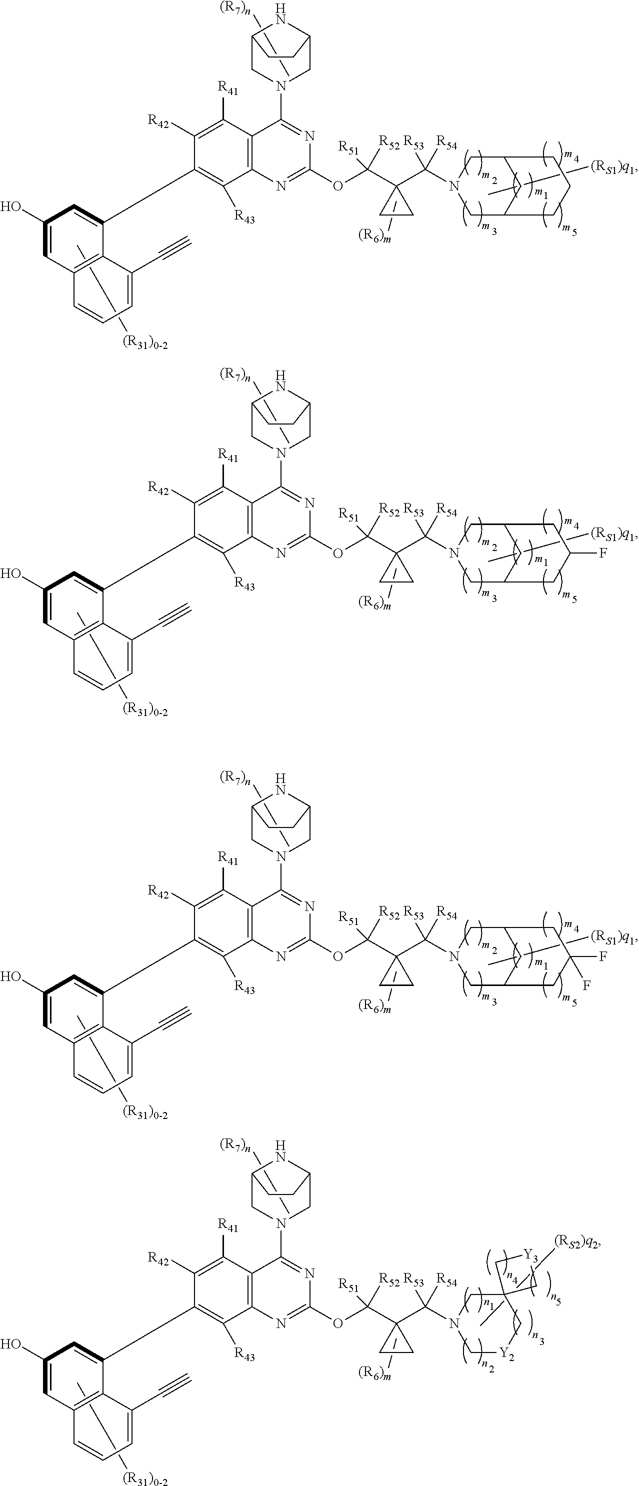

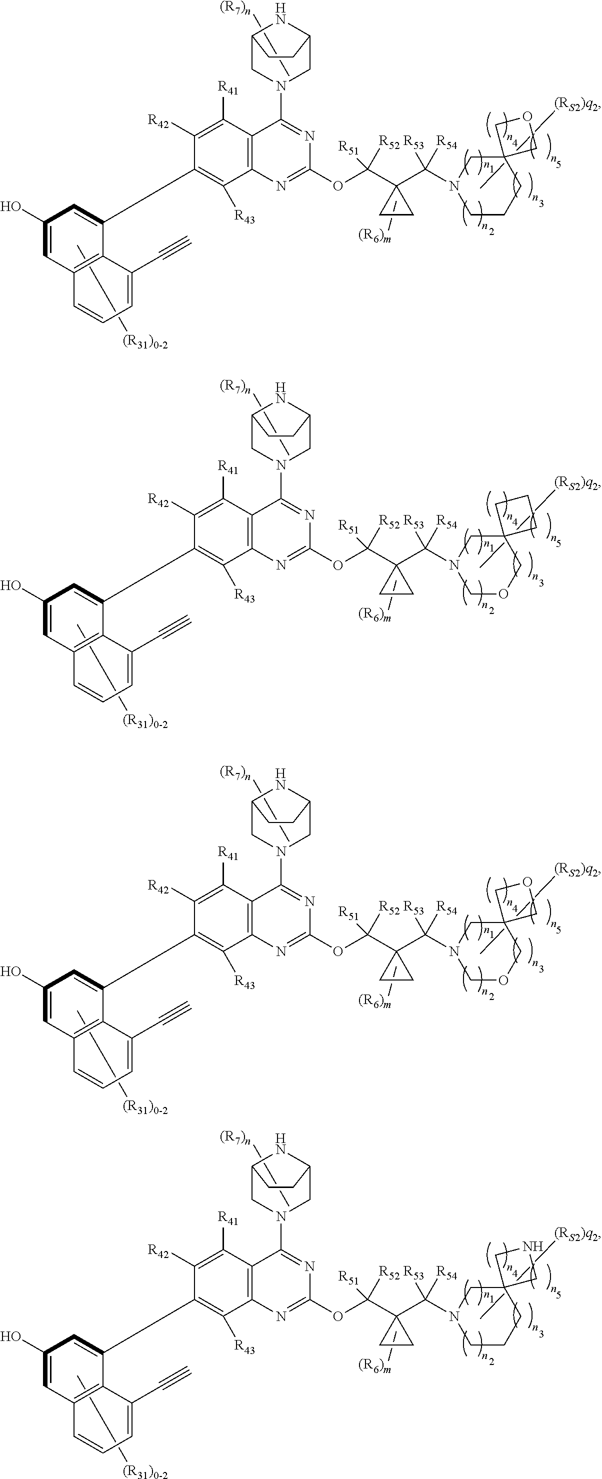

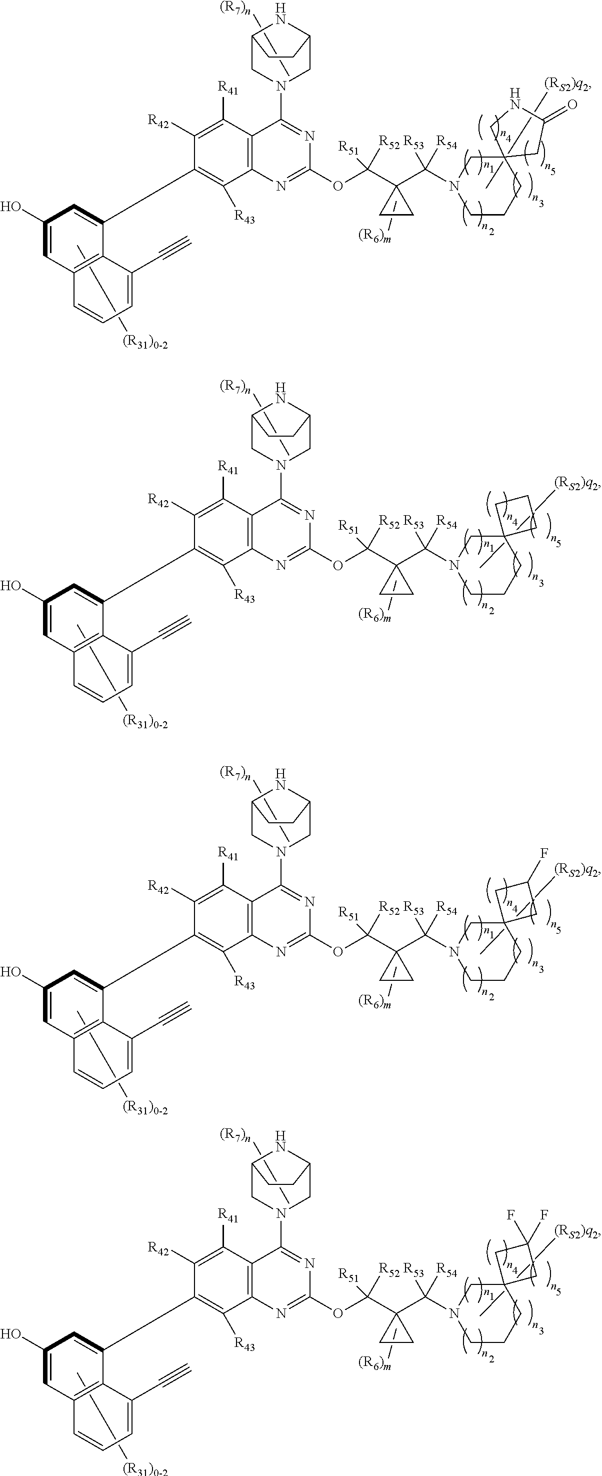

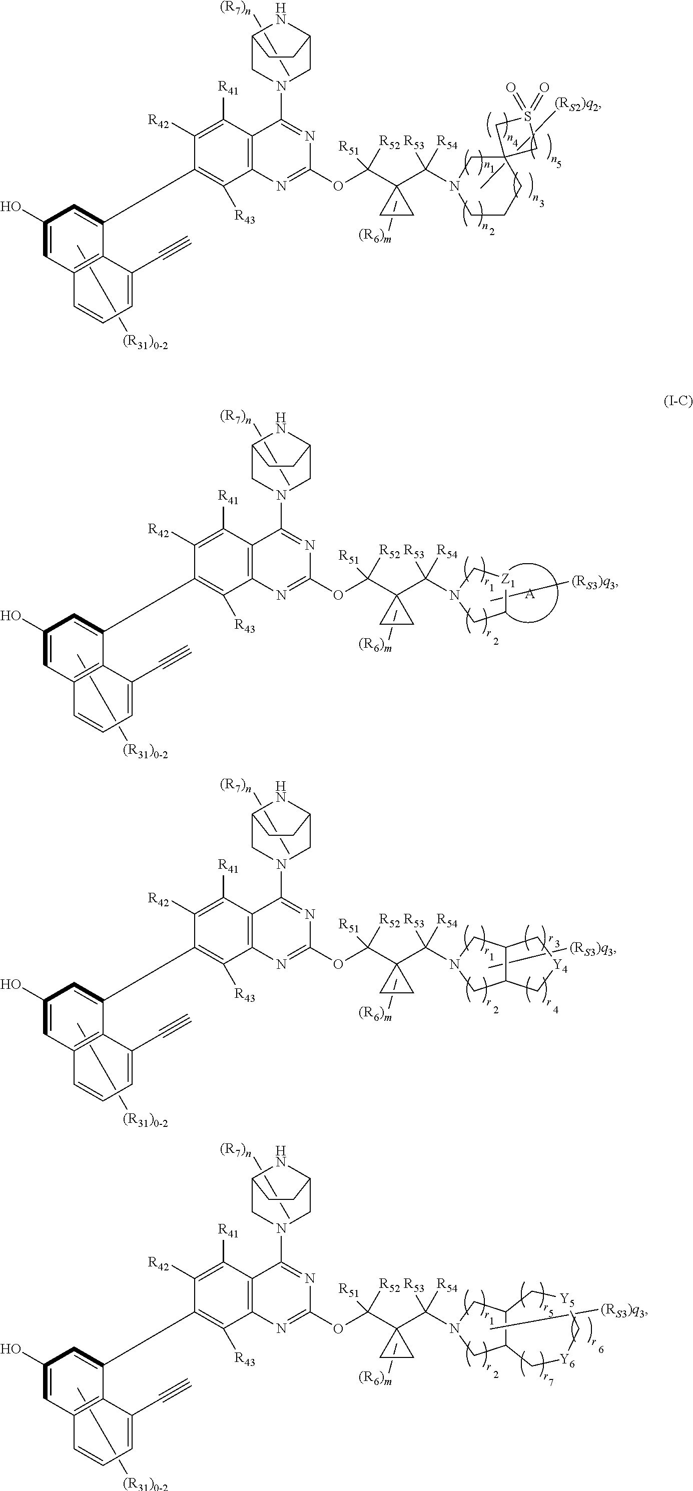

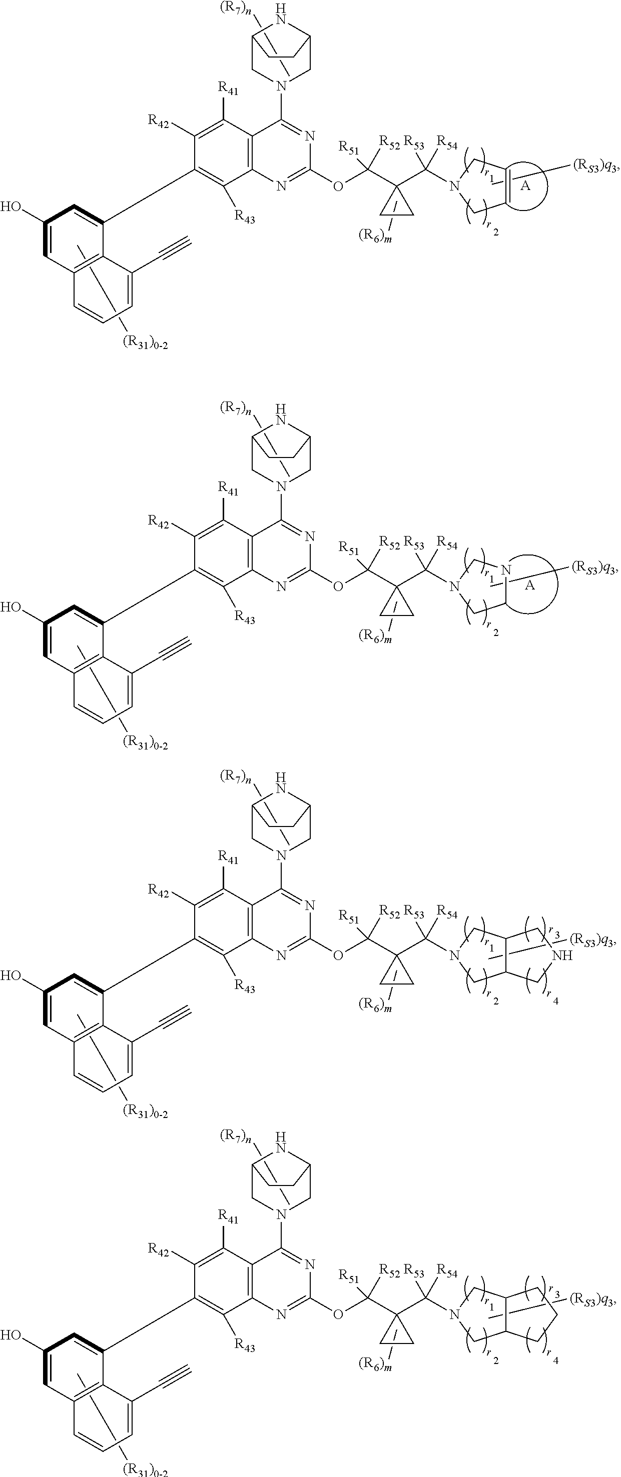

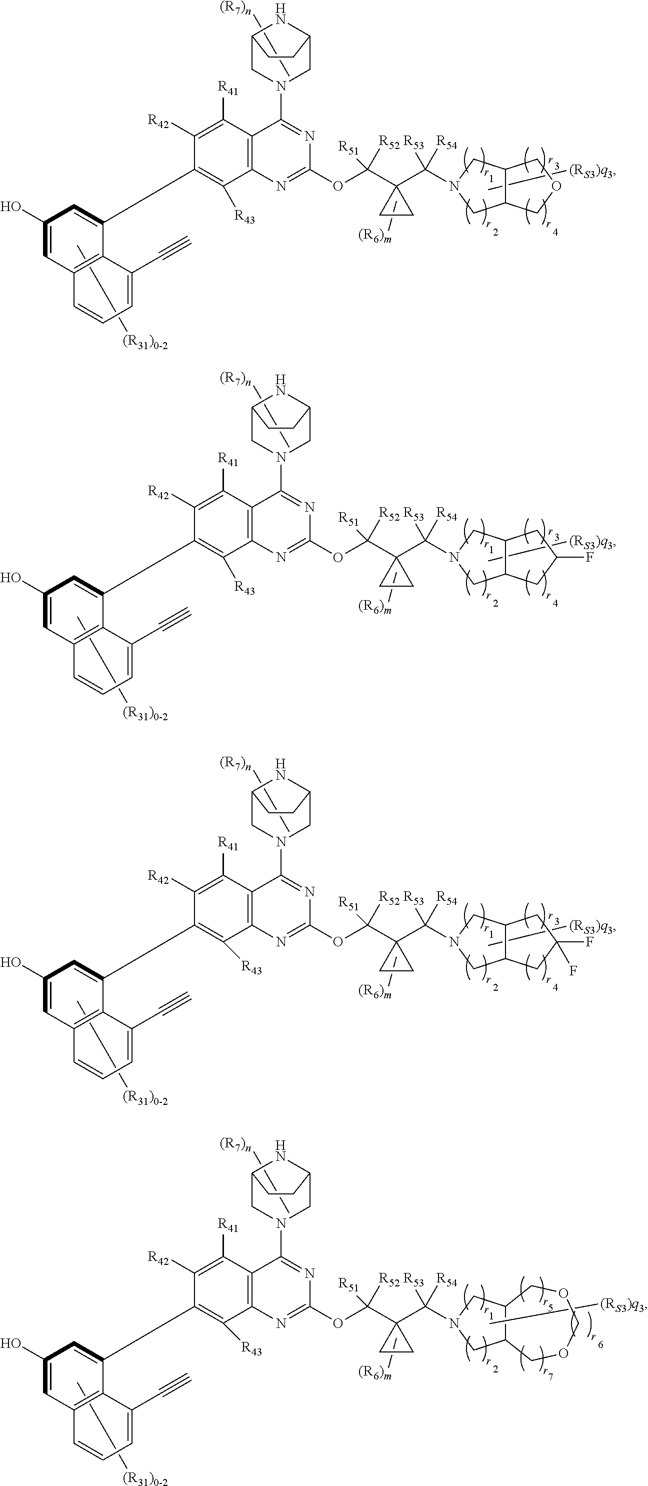

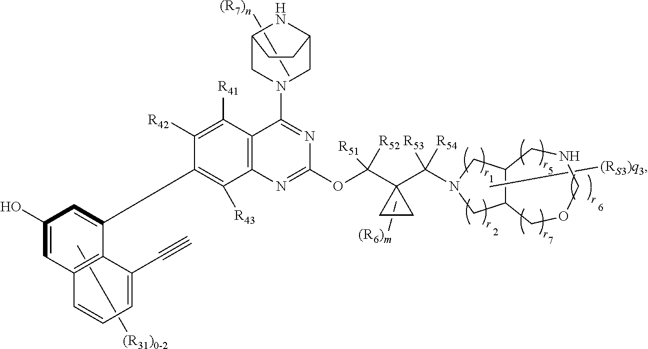

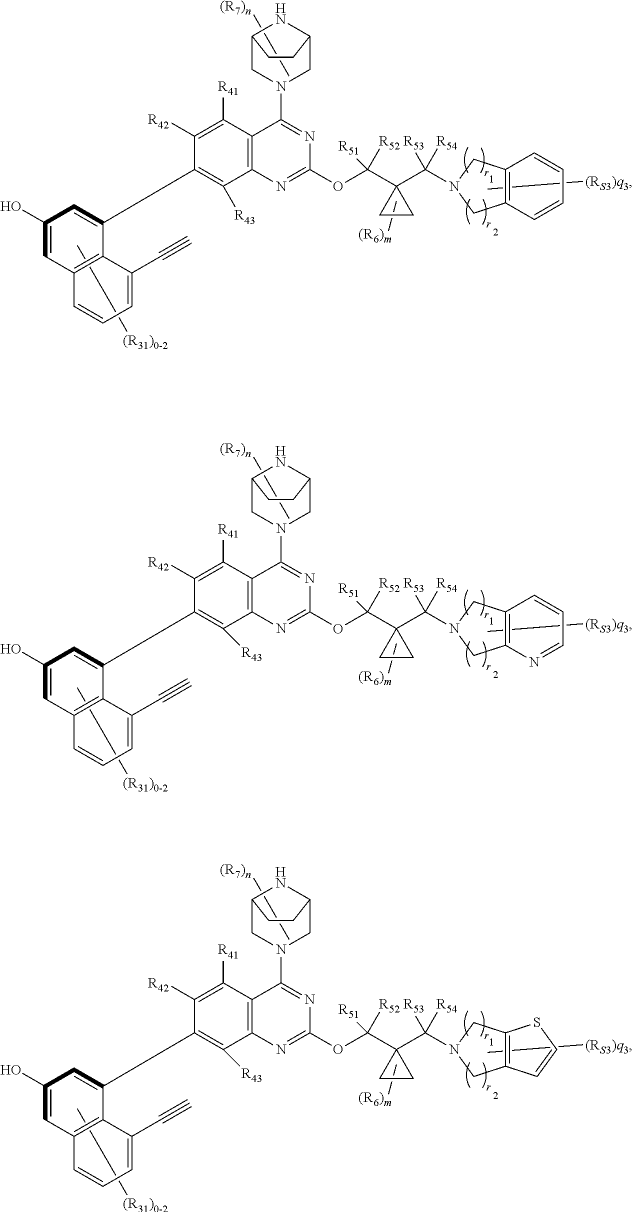

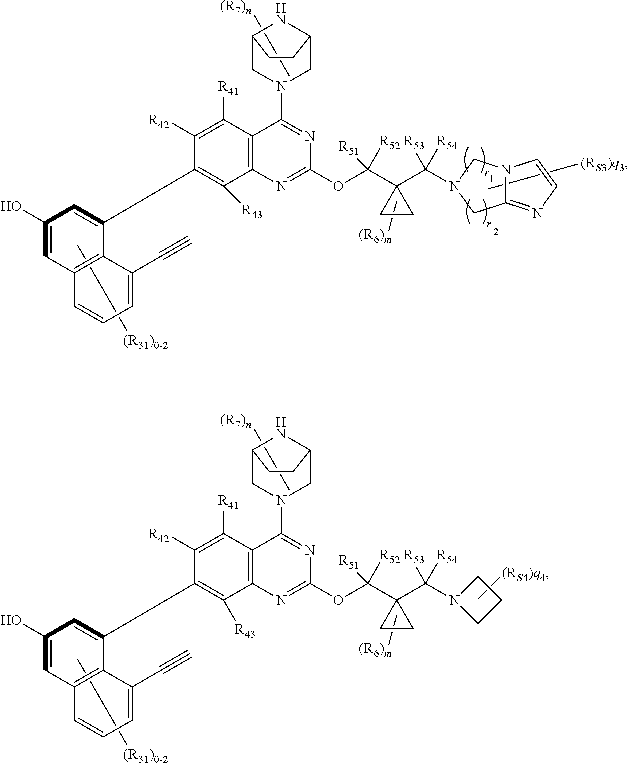

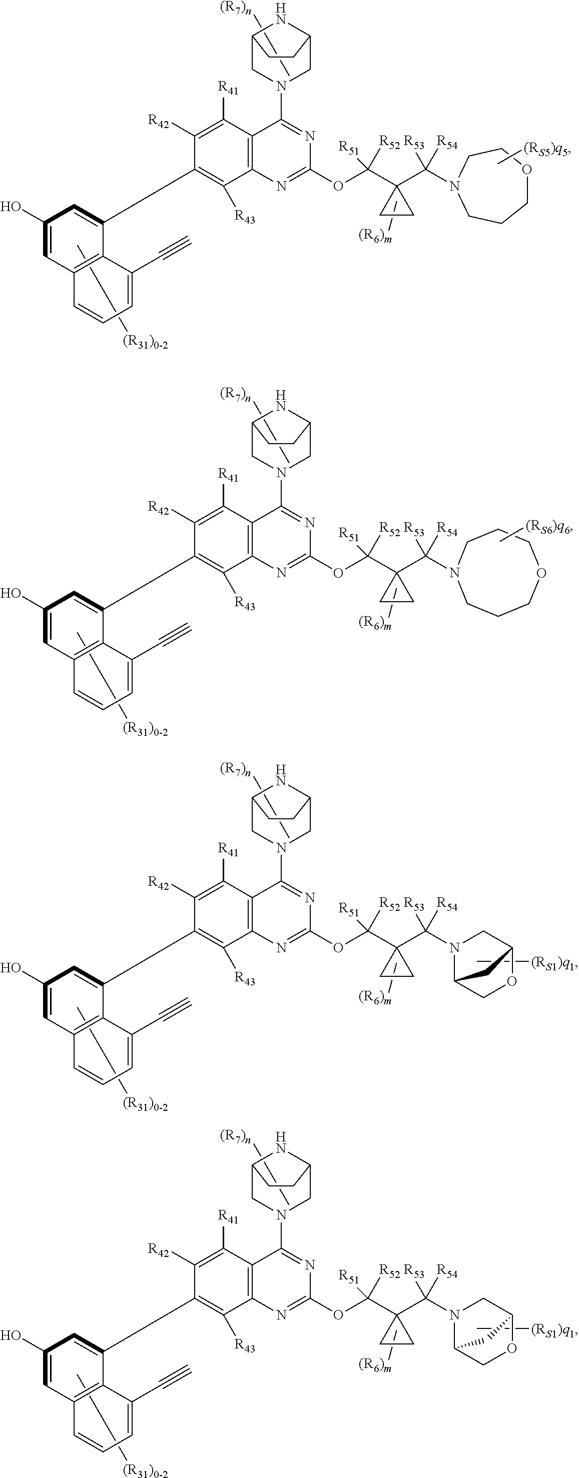

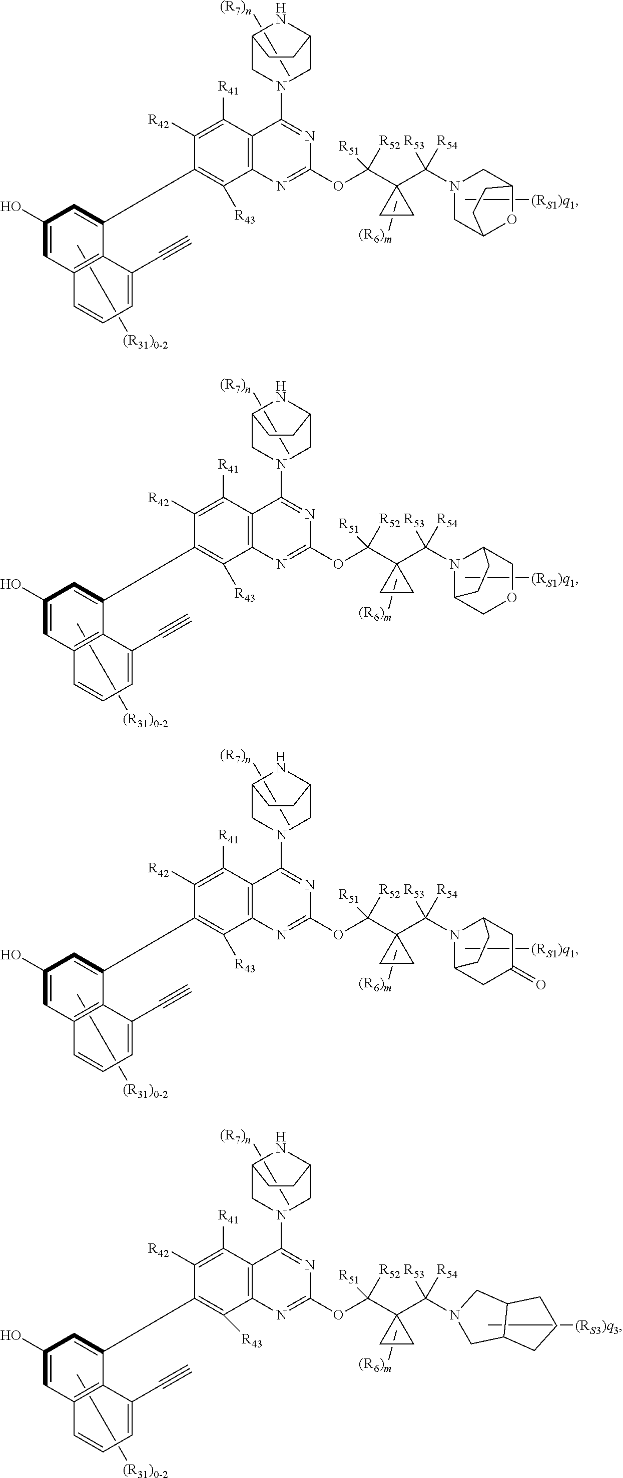

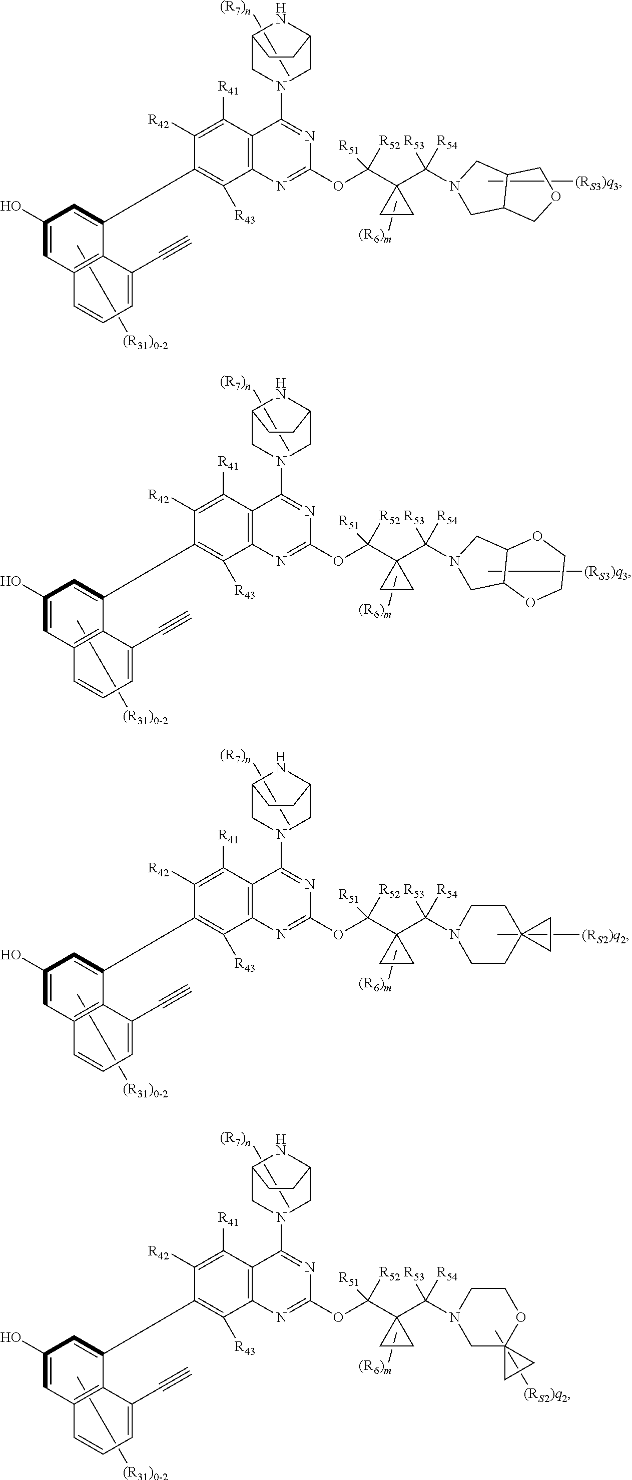

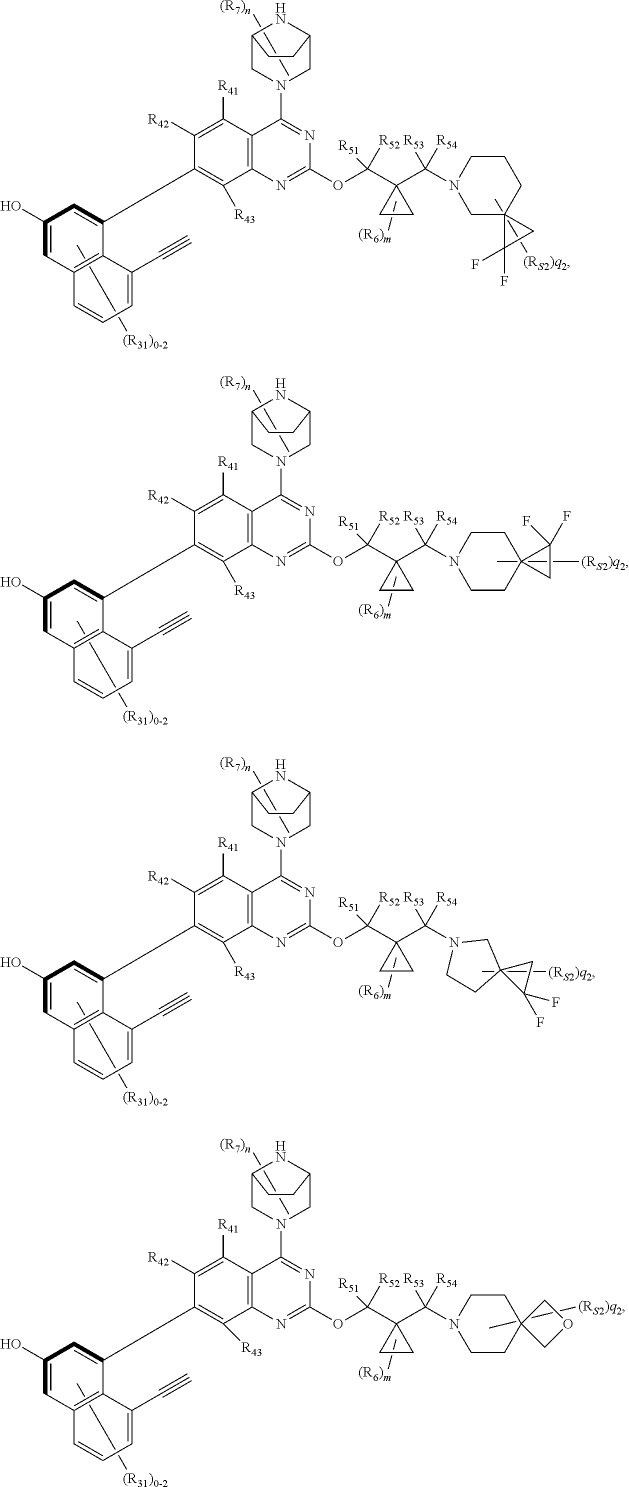

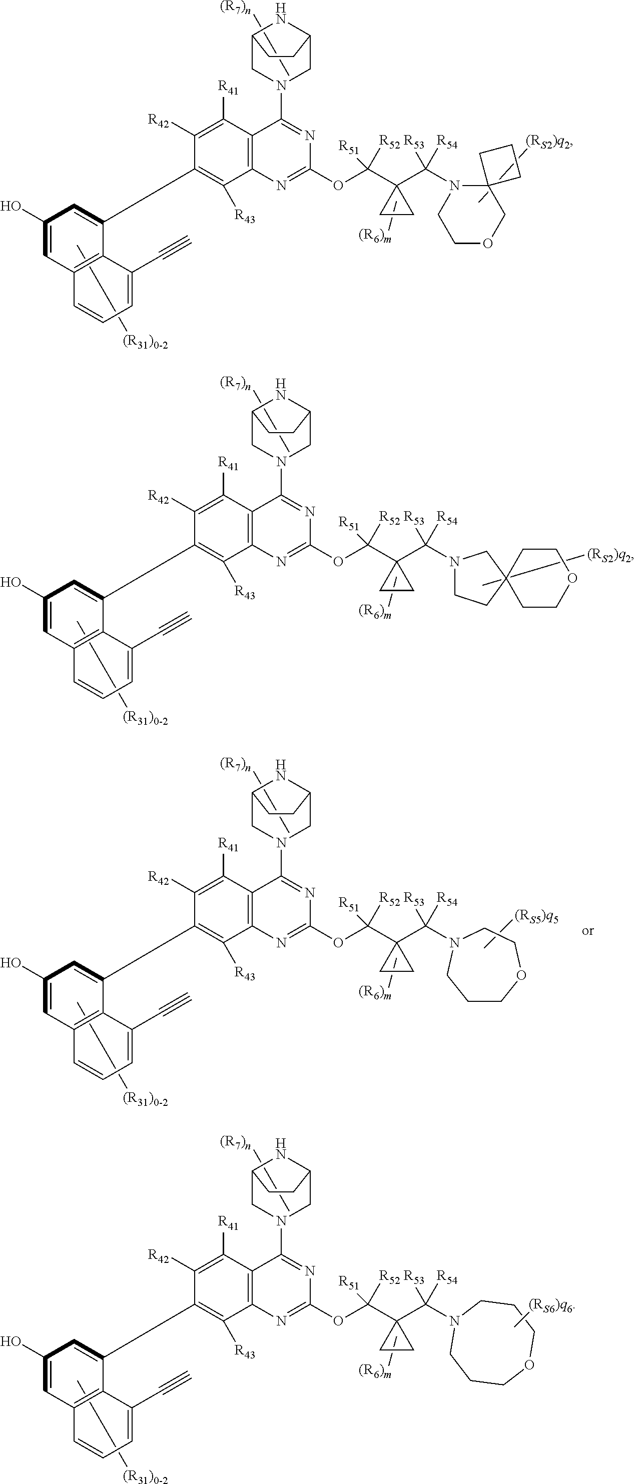

[100]. The compound according to any one of [1] to [99], wherein, R₄₁, R₄₂ or R₄₃ at each occurrence is independently selected from hydrogen, halogen, —C₁₋₄alkyl, haloC₁₋₃alkyl, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(haloC₁₋₃alkyl), —S(═O)C₁₋₃alkyl, —S(═O)₂C₁₋₃alkyl, —COOH, —C(═O)C₁₋₃alkyl, —C(═O)NH₂, —C(═O)NHC₁₋₃alkyl, —C(═O)N(C₁₋₃alkyl)₂, —S(═O)₂NH₂, —S(═O)₂NHC₁₋₃alkyl, —S(═O)₂N(C₁₋₃alkyl)₂, —P(═O)H(C₁₋₃alkyl), —P(═O)(C₁₋₃alkyl)₂, 3-6 membered cycloalkyl or 3-6 membered heterocyclyl; wherein, said —C₁₋₃alkyl, —C₁₋₄alkyl, 3-6 membered cycloalkyl, or 3-6 membered heterocyclyl is independently optionally substituted with 1, 2, 3, 4, 5 or 6 substituents selected from —F, —C₁₋₃alkyl, —CN, —NH₂, —NH(C₁₋₃alkyl), —N(C₁₋₃alkyl)₂, —OH, —O(C₁₋₃alkyl), —SH, —S(C₁₋₃alkyl) or 3-6 membered cycloalkyl or 3-6 membered heterocyclyl;

Preferably, R₄₁, R₄₂ or R₄₃ at each occurrence is independently selected from —H, —Cl, —F, —Br, —CH₃, —CH₂CH₃, —CH₂CH₂CH₃, —CH(CH₃)₂, —C(CH₃)₃,

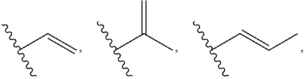

—CH₂F, —CHF₂, —CF₃, —CH₂CH₂F, —CH₂CHF₂, —CH₂CF₃, —CHFCH₃, —CF₂CH₃, —CN, —NH₂, —NH(CH₃), —N(CH₃)₂, —NH(CH₂CH₃), —OH, —O—CH₃, —O—CH₂CH₃, —O—CH₂CH₂CH₃, —O—CH(CH₃)₂, —SH, —S—CH₃, —S—CH₂CH₃, —S—CH₂CH₂CH₃, —S—CH(CH₃)₂, —S(═O)CH₃, —S(═O)(CH₂CH₃), —S(═O)(CH₂CH₂CH₃), —S(═O)(CH(CH₃)₂), —S(═O)₂CH₃, —S(═O)₂(CH₂CH₃), —S(═O)₂(CH₂CH₂CH₃), —S(═O)₂(CH(CH₃)₂), —P(═O)(CH₃)₂, —O—CH₂F, —O—CHF₂, —OCF₃, —SCH₂F, —SCHF₂, —SCF₃, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, —COOH, —C(═O)(CH₃), —C(═O)(CH₂CH₃), —C(═O)(CH(CH₃)₂), —C(═O)(CF₃),

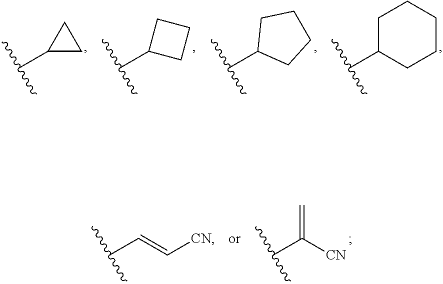

Preferably, R₄₁ at each occurrence is independently selected from —H;

Preferably, R₄₂ at each occurrence is independently selected from —H, —Cl, —F, —CH₃, —CH₂CH₃, —CH(CH₃)₂, —C(CH₃)₃,

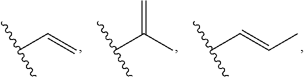

—CF₃, —CN, —NH₂, —NH(CH₃), —N(CH₃)₂, —OH, —OCH₃, —S(═O)CH₃, —S(═O)₂CH₃, —P(═O)(CH₃)₂, —SCF₃, —CH₂OH, —CH₂CH₂CN, —C(═O)(CH₃),

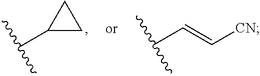

Preferably, R₄₃ at each occurrence is independently selected from —H, —Cl, —F, —CH₃, —CH₂CH₃, —CH(CH₃)₂, —C(CH₃)₃,

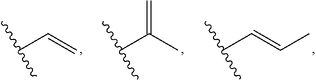

—CF₃, —CN, —NH₂, —NH(CH₃), —N(CH₃)₂, —OH, —OCH₃, —S(═O)CH₃, —S(═O)₂CH₃, —P(═O)(CH₃)₂, —S—CF₃, —CH₂—OH, —CH₂CH₂—CN, —COOH, —C(═O)(CH₃),

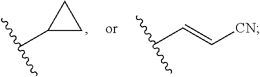

More preferably, R₄₁ at each occurrence is independently selected from —H; R₄₂ at each occurrence is independently selected from —H, —Cl, —F, —CH₃, —CH₂CH₃, —CH(CH₃)₂, —C(CH₃)₃,

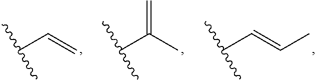

—CF₃, —CN, —NH₂, —NH(CH₃), —N(CH₃)₂, —OH, —O—CH₃, —S(═O)CH₃, —S(═O)₂CH₃, —P(═O)(CH₃)₂, —S—CF₃, —CH₂—OH, —CH₂CH₂—CN, —C(═O)(CH₃),

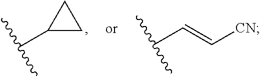

R₄₃ at each occurrence is independently selected from —H, —Cl, —F, —CH₃, —CH₂CH₃, —CH(CH₃)₂, —C(CH₃)₃,

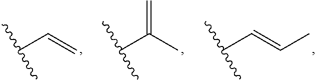

—CF₃, —CN, —NH₂, —NH(CH₃), —N(CH₃)₂, —OH, —O—CH₃, —S(═O)CH₃, —S(═O)₂CH₃, —P(═O)(CH₃)₂, —S—CF₃, —CH₂—OH, —CH₂CH₂—CN, —COOH, —C(═O)(CH₃),

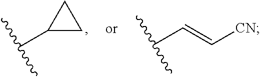

[101]. The compound according to any one of [1] to [100], wherein, R₅₁, R₅₂, R₅₃, R₅₄, or R₅₅ at each occurrence is independently selected from hydrogen, —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl, wherein said —C₁₋₃alkyl, —C₁₋₃haloalkyl, —C₁₋₃haloalkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is independently optionally substituted with 1, 2, 3, 4, 5 or 6 substituents selected from hydrogen, —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;

Preferably, R₅₁, R₅₂, R₅₃, R₅₄ or R₅₅ at each occurrence is independently selected from —H, —Cl, —F, —Br, —CH₃, —CH₂CH₃, —CH₂CH₂CH₃, —CH(CH₃)₂, —C(CH₃)₃, —O—CH₂F, —O—CHF₂, —O—CF₃, —S—CH₂F, —S—CHF₂, —S—CF₃,

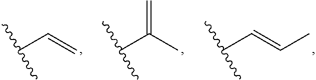

—CH₂F, —CHF₂, —CF₃, —CH₂CH₂F, —CH₂CHF₂, —CH₂CF₃, —CHFCH₃, —CF₂CH₃, —CN, oxo, —NH₂, —NH(CH₃), —N(CH₃)₂, —NH(CH₂CH₃), —OH, —O—CH₃, —O—CH₂CH₃, —O—CH₂CH₂CH₃, —O—CH(CH₃)₂, —SH, —S—CH₃, —S—CH₂CH₃, —S—CH₂CH₂CH₃, —S—CH(CH₃)₂, —S(═O)CH₃, —S(═O)(CH₂CH₃), —S(═O)(CH₂CH₂CH₃), —S(═O)(CH(CH₃)₂), —S(═O)₂CH₃, —S(═O)₂(CH₂CH₃), —S(═O)₂(CH₂CH₂CH₃), —S(═O)₂(CH(CH₃)₂), —COOH, —C(═O)(CH₃), —C(═O)(CH₂CH₃), —C(═O)(CH(CH₃)₂), —C(═O)(CF₃), —C(═O)(OCH₃), —C(═O)(OCH₂CH₃), —C(═O)(OCH₂CH₂CH₃), —C(═O)(OCH(CH₃)₂), —OC(═O)(CH₃), —OC(═O)(CH₂CH₃), —OC(═O)(CH₂CH₂CH₃), —OC(═O)(CH(CH₃)₂), —C(═O)NH₂, —C(═O)NH(CH₃), —C(═O)NH(CH₂CH₃), —C(═O)NH(CH₂CH₂CH₃), —C(═O)NH(CH(CH₃)₂), —C(═O)N(CH₃)₂, —C(═O)N(CH₂CH₃)₂, —NHC(═O)(CH₃), —NHC(═O)(CH₂CH₃), —NHC(═O)(CH₂CH₂CH₃), —NHC(═O)(CH(CH₃)₂), —N(CH₃)C(═O)(CH₃), —S(═O)(OCH₃), —S(═O)(OCH₂CH₃), —S(═O)(OCH₂CH₂CH₃), —S(═O)(OCH(CH₃)₂), —OS(═O)(CH₃), —OS(═O)(CH₂CH₃), —OS(═O)(CH₂CH₂CH₃), —OS(═O)(CH(CH₃)₂), —S(═O)NH₂, —S(═O)NH(CH₃), —S(═O)NH(CH₂CH₃), —S(═O)NH(CH₂CH₂CH₃), —S(═O)NH(CH(CH₃)₂), —S(═O)N(CH₃)₂, —S(═O)N(CH₃)(CH₂CH₃), —NHS(═O)(CH₃), —NHS(═O)(CH₂CH₃), —NHS(═O)(CH₂CH₂CH₃), —NHS(═O)(CH(CH₃)₂), —N(CH₃)S(═O)(CH₃), —S(═O)₂(OCH₃), —S(═O)₂(OCH₂CH₃), —S(═O)₂(OCH₂CH₂CH₃), —S(═O)₂(OCH(CH₃)₂), —OS(═O)₂(CH₃), —OS(═O)₂(CH₂CH₃), —OS(═O)₂(CH₂CH₂CH₃), —OS(═O)₂(CH(CH₃)₂), —S(═O)₂NH₂, —S(═O)₂NH(CH₃), —S(═O)₂NH(CH₂CH₃), —S(═O)₂NH(CH₂CH₂CH₃), —S(═O)₂NH(CH(CH₃)₂), —S(═O)₂N(CH₃)₂, —S(═O)₂N(CH₃)(CH₂CH₃), —NHS(═O)₂(CH₃), —NHS(═O)₂(CH₂CH₃), —NHS(═O)₂(CH₂CH₂CH₃), —NHS(═O)₂(CH(CH₃)₂), —N(CH₃)S(═O)₂(CH₃), —P(═O)H(CH₃), —P(═O)H(CH₂CH₃), —P(═O)H(CH₂CH₂CH₃), —P(═O)H(CH(CH₃)₂), —P(═O)(CH₃)₂, —P(═O)(CH₃)(CH₂CH₃), —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN,

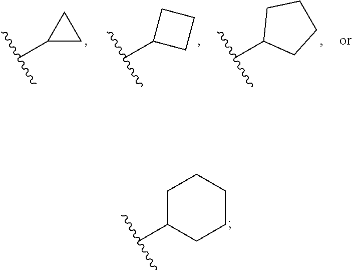

More preferably, R₅₁, R₅₂, R₅₃ or R₅₄ at each occurrence is independently selected from —H, —Cl, —F, —Br, —CH₃, —CH₂CH₃, —CH(CH₃)₂, —C(CH₃)₃, —O—CF₃, —S—CF₃, —CF₃, —CN, oxo, —NH₂, —NH(CH₃), —N(CH₃)₂, —OH, —O—CH₃, —O—CH(CH₃)₂, —SH, —S—CH₃, —S—CH(CH₃)₂, —S(═O)CH₃, —S(═O)₂CH₃, —COOH, —C(═O)(CH₃), —C(═O)(CH₂CH₃), —C(═O)(CF₃), —C(═O)NH₂, —C(═O)NH(CH₃), —NHC(═O)(CH₃), —S(═O)NH₂, —S(═O)NH(CH₃), —NHS(═O)(CH₃), —S(═O)₂NH₂, —S(═O)₂NH(CH₃), —NHS(═O)₂(CH₃), —P(═O)H(CH₃), —P(═O)(CH₃)₂, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, or

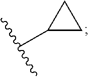 [SMILES: *C1CC1]

Further preferably, R₅₁, R₅₂, R₅₃, R₅₄ or R₅₅ at each occurrence is independently selected from —H.

[102]. The compound according to any one of [1] to [101], wherein, R₆ at each occurrence is independently selected from —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl, wherein said —C₁₋₃alkyl, —C₁₋₃haloalkyl, —C₁₋₃haloalkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is independently optionally substituted with 1, 2, 3, 4, 5 or 6 substituents selected from hydrogen, —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; preferably, R₆ at each occurrence is independently selected from —H, —Cl, —F, —Br, —CH₃, —CH₂CH₃, —CH(CH₃)₂, —C(CH₃)₃, —O—CF₃, —S—CF₃, —CF₃, —CN, oxo, —NH₂, —NH(CH₃), —N(CH₃)₂, —OH, —O—CH₃, —O—CH(CH₃)₂, —SH, —S—CH₃, —S—CH(CH₃)₂, —S(═O)CH₃, —S(═O)₂CH₃, —COOH, —C(═O)(CH₃), —C(═O)(CH₂CH₃), —C(═O)(CF₃), —C(═O)NH₂, —C(═O)NH(CH₃), —NHC(═O)(CH₃), —S(═O)NH₂, —S(═O)NH(CH₃), —NHS(═O)(CH₃), —S(═O)₂NH₂, —S(═O)₂NH(CH₃), —NHS(═O)₂(CH₃), —P(═O)H(CH₃), —P(═O)(CH₃)₂, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, or

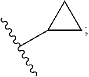 [SMILES: *C1CC1]

m is selected from 0, 1, 2, 3, 4, 5, or 6; preferably, m is selected from 0, 1, 2, or 3, more preferably, m is selected from 0.

[103]. The compound according to any one of [1] to [102], wherein, R₇ at each occurrence is independently selected from —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl, wherein said —C₁₋₃alkyl, —C₁₋₃haloalkyl, —C₁₋₃haloalkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is independently optionally substituted with 1, 2, 3, 4, 5 or 6 substituents selected from hydrogen, —F, —Cl, —Br, —C₁₋₃alkyl, —C₁₋₃haloalkyl, —C₁₋₃haloalkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; preferably, R₇ at each occurrence is independently selected from —H, —Cl, —F, —Br, —CH₃, —CH₂CH₃, —CH(CH₃)₂, —C(CH₃)₃, —O—CF₃, —S—CF₃, —CF₃, —CN, oxo, —NH₂, —NH(CH₃), —N(CH₃)₂, —OH, —O—CH₃, —O—CH(CH₃)₂, —SH, —S—CH₃, —S—CH(CH₃)₂, —S(═O)CH₃, —S(═O)₂CH₃, —COOH, —C(═O)(CH₃), —C(═O)(CH₂CH₃), —C(═O)(CF₃), —C(═O)NH₂, —C(═O)NH(CH₃), —NHC(═O)(CH₃), —S(═O)NH₂, —S(═O)NH(CH₃), —NHS(═O)(CH₃), —S(═O)₂NH₂, —S(═O)₂NH(CH₃), —NHS(═O)₂(CH₃), —P(═O)H(CH₃), —P(═O)(CH₃)₂, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, or

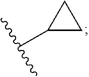 [SMILES: *C1CC1]

n is selected from 0, 1, 2, 3, 4, 5, or 6; preferably, n is selected from 0, 1, 2, or 3, more preferably, n is selected from 0.

[104]. The compound according to any one of [1] to [103], wherein, the compound is selected from:

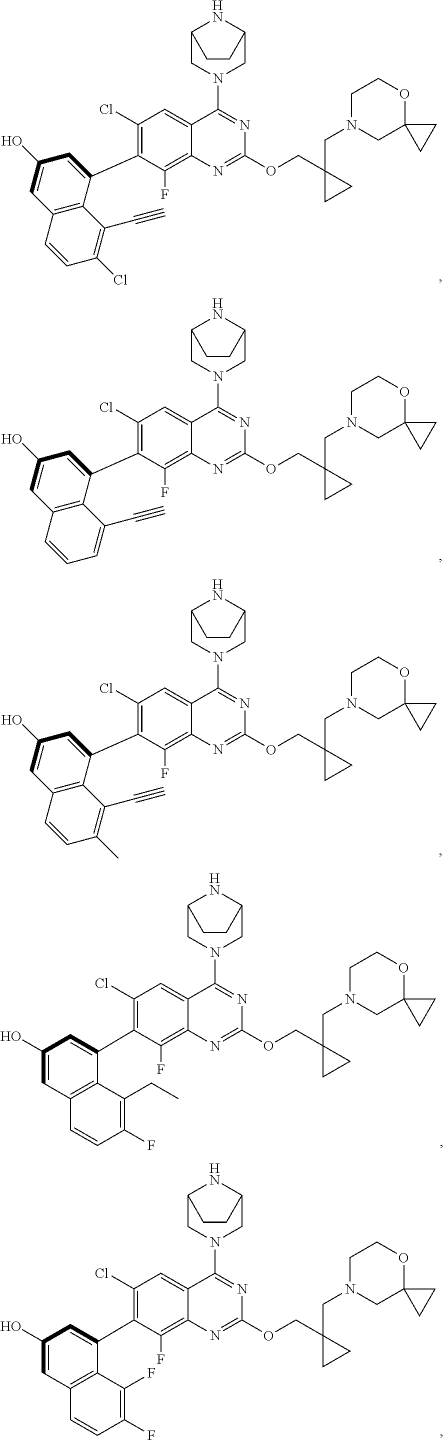

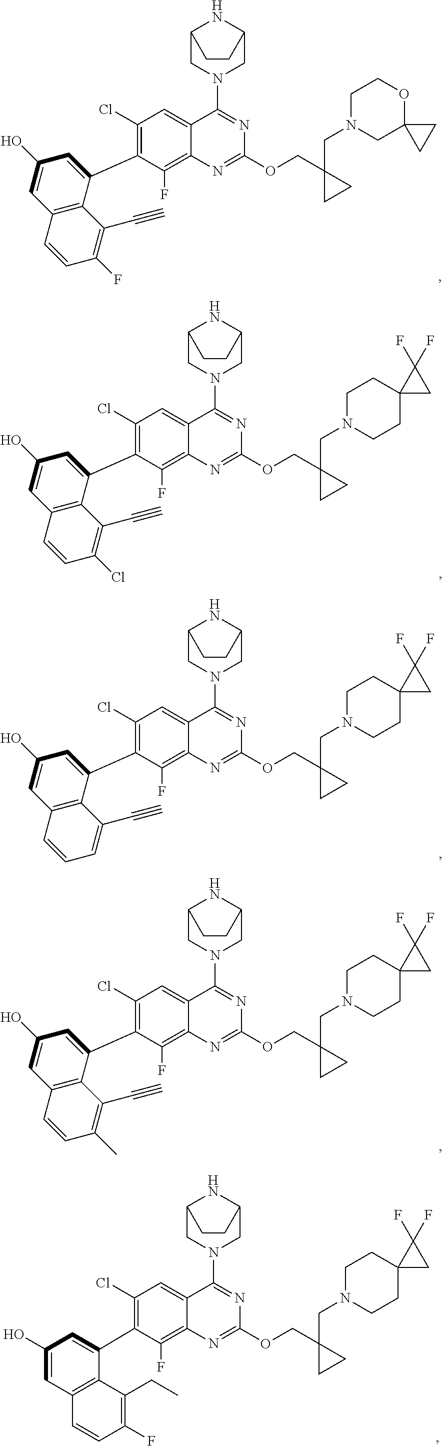

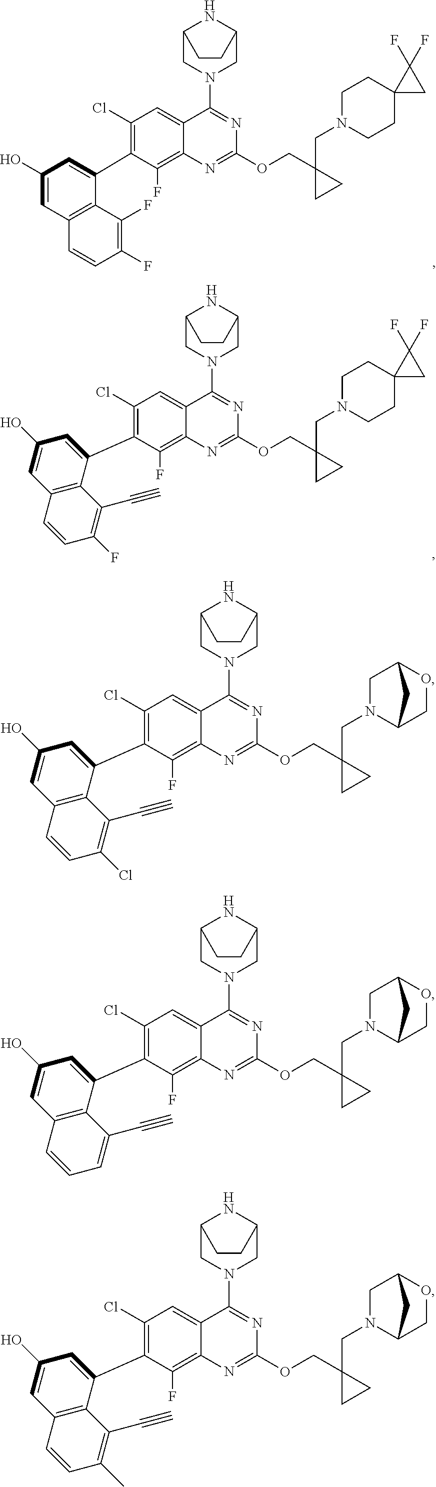

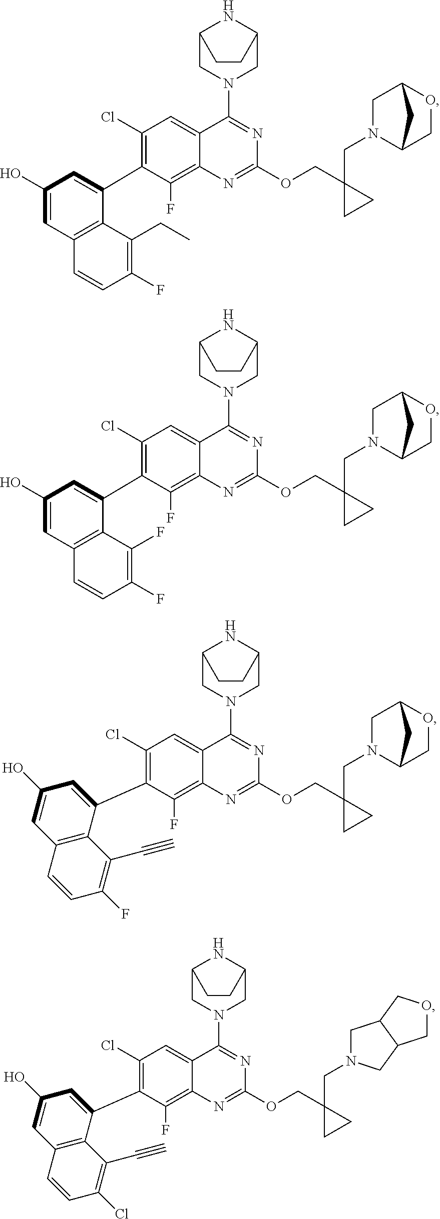

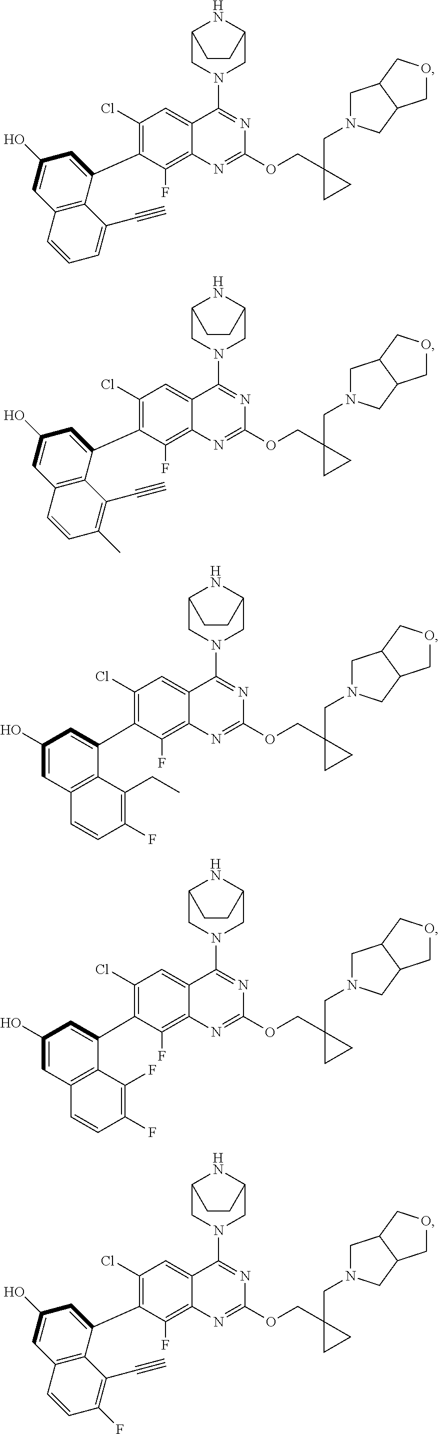

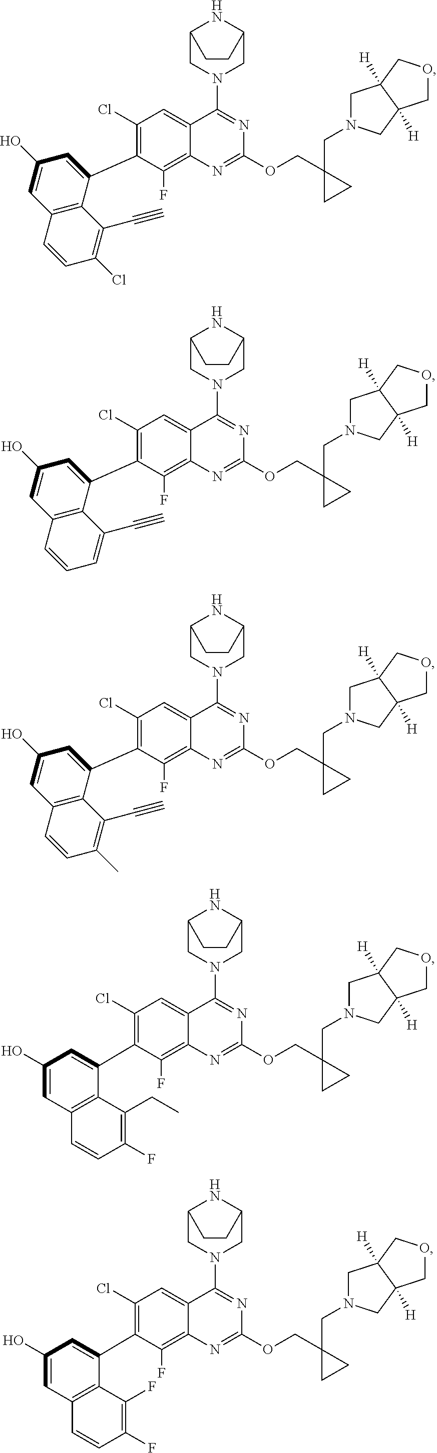

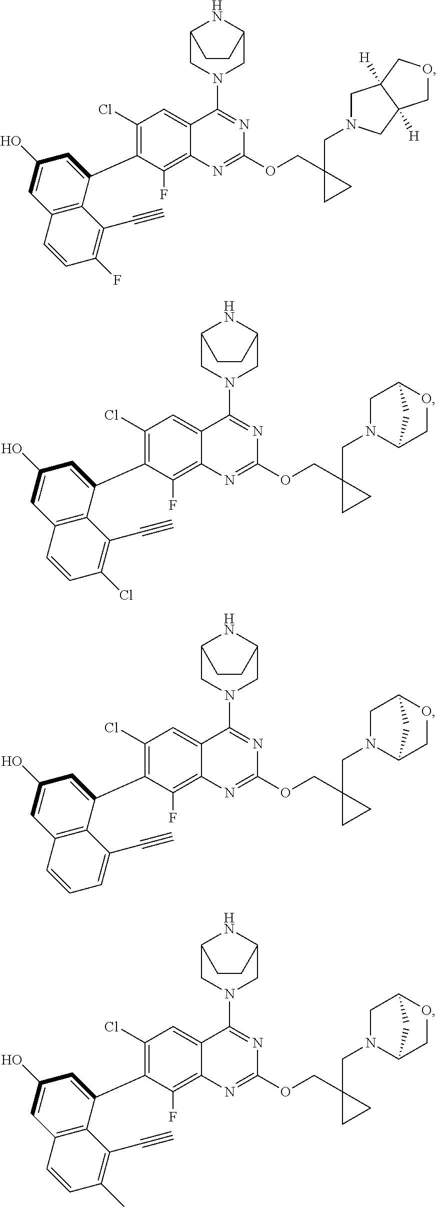

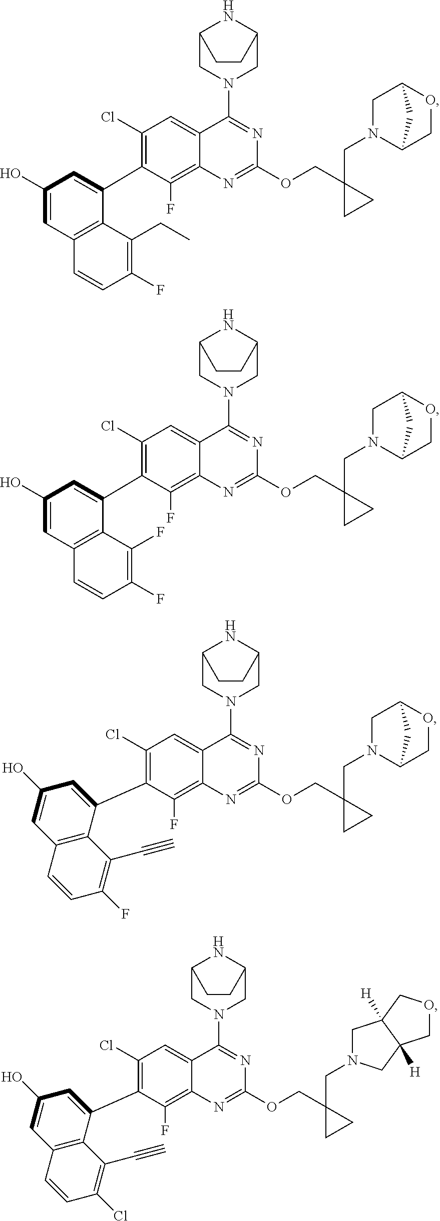

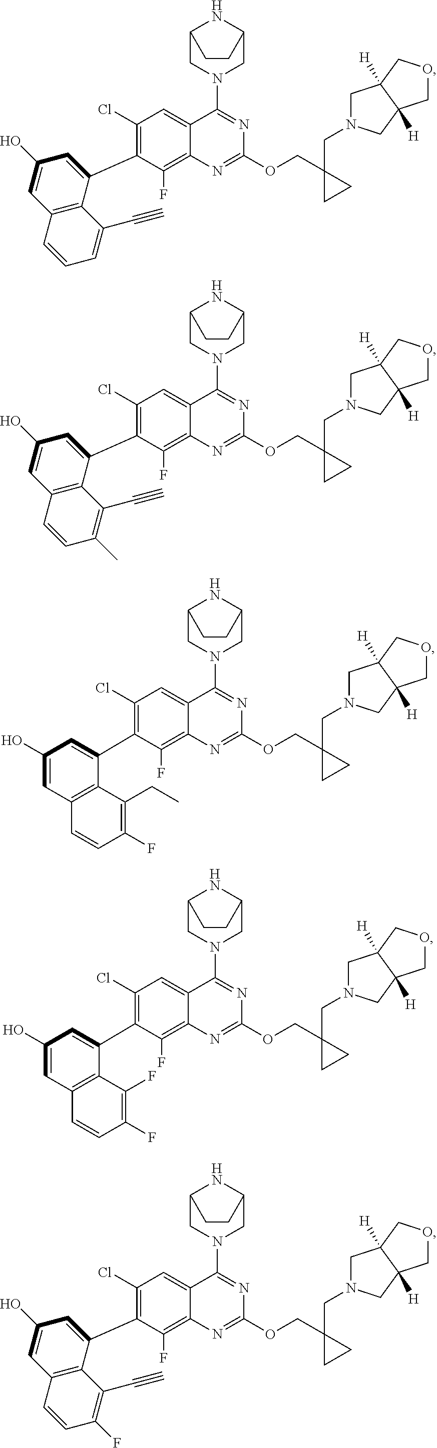

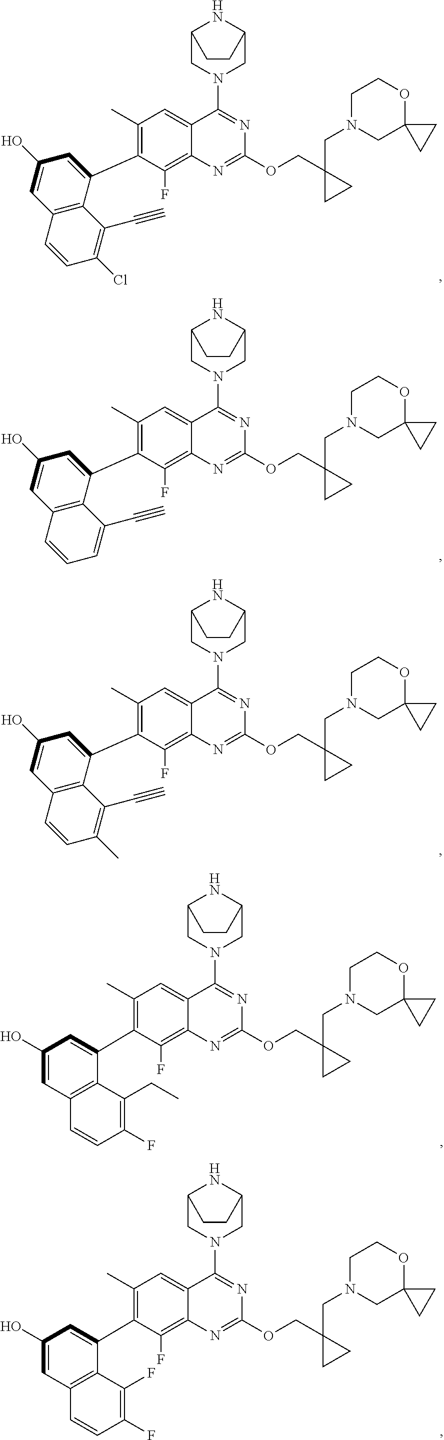

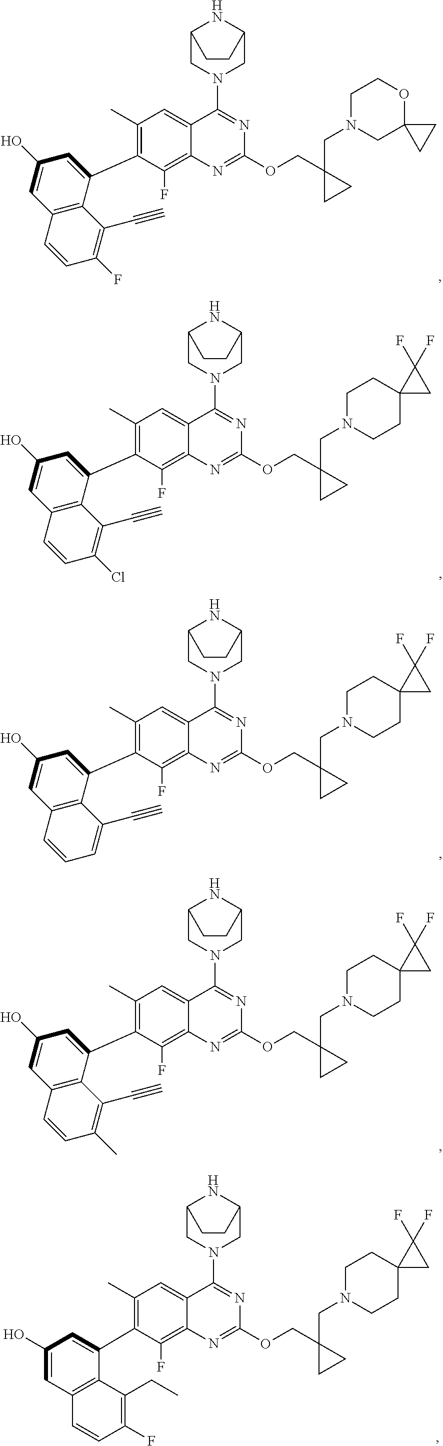

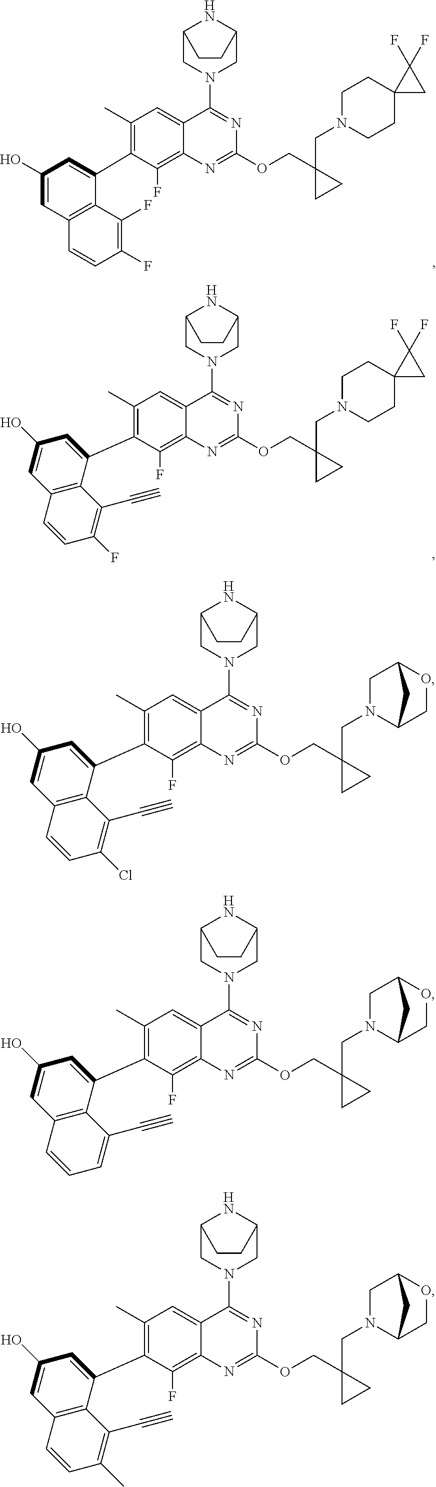

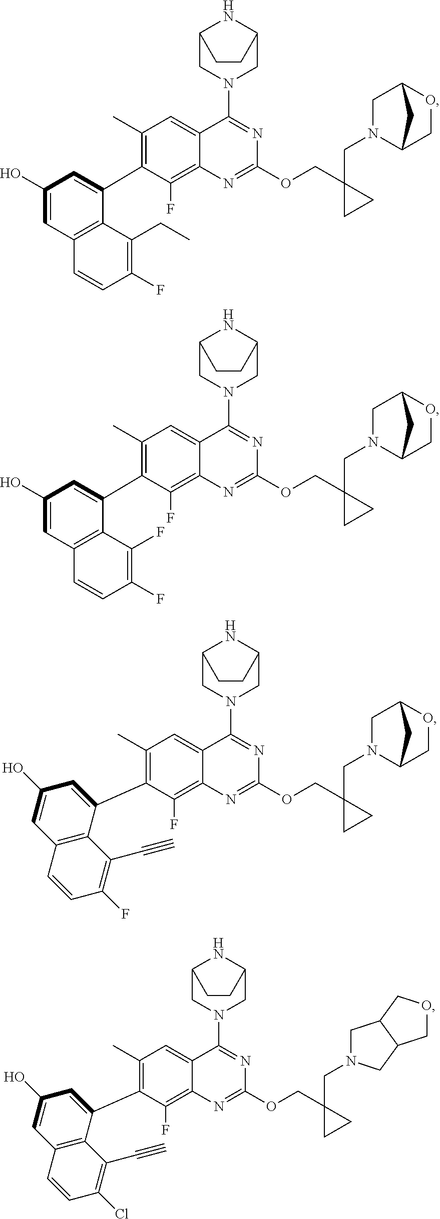

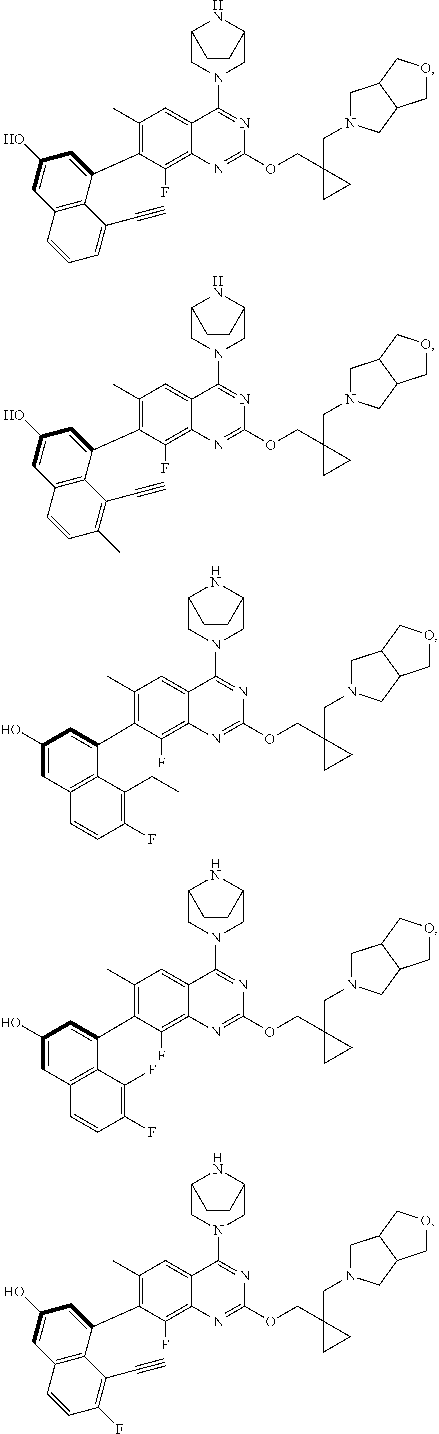

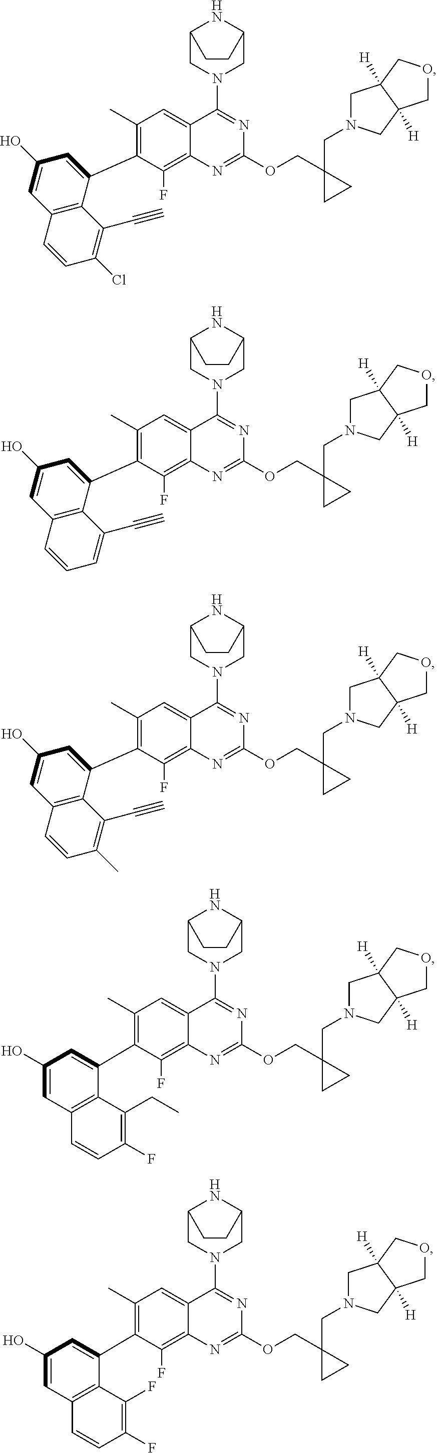

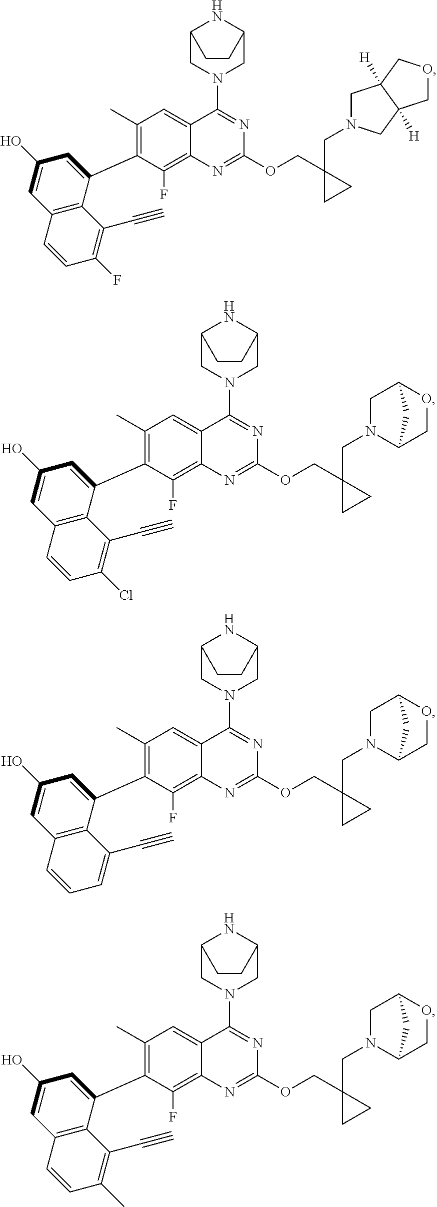

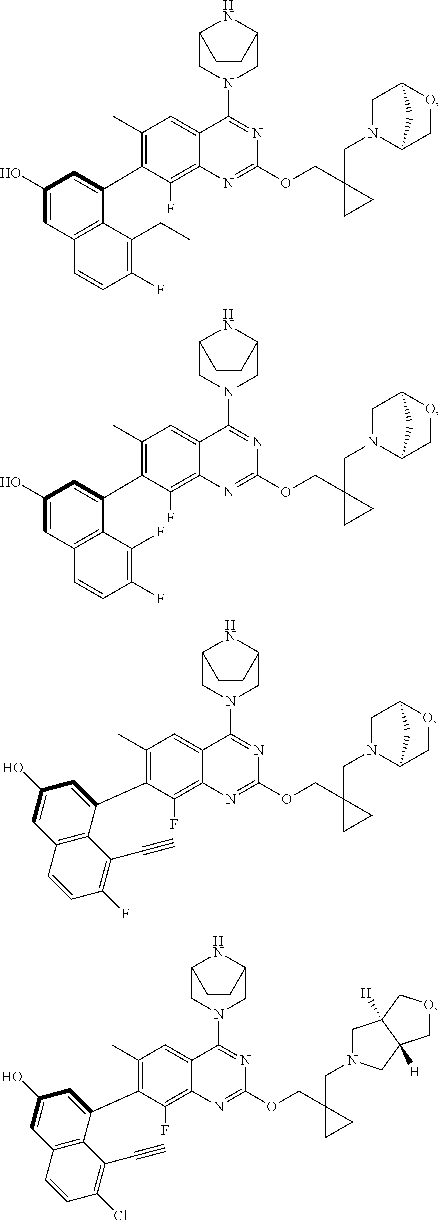

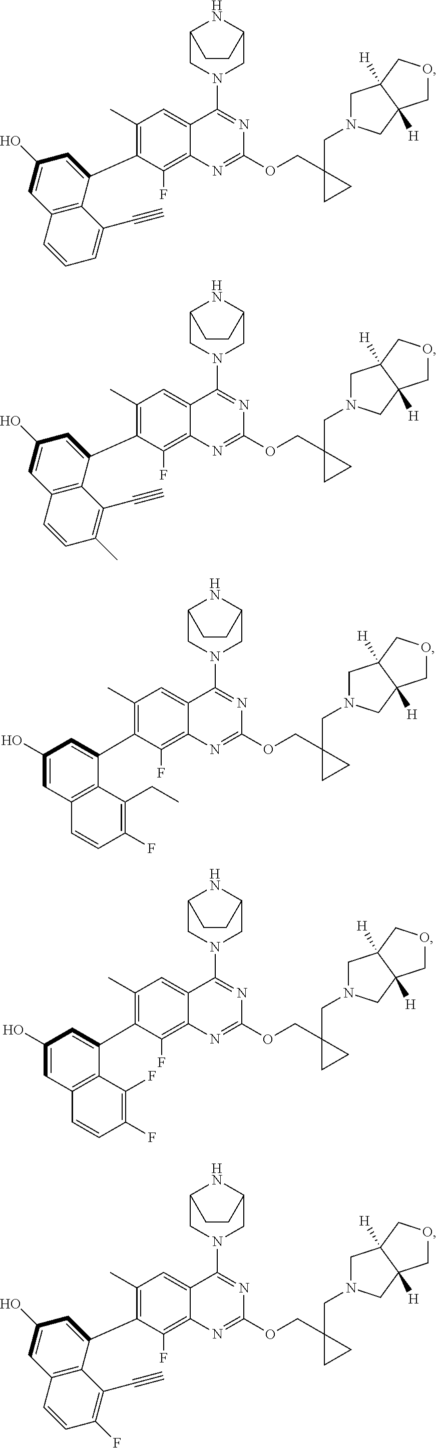

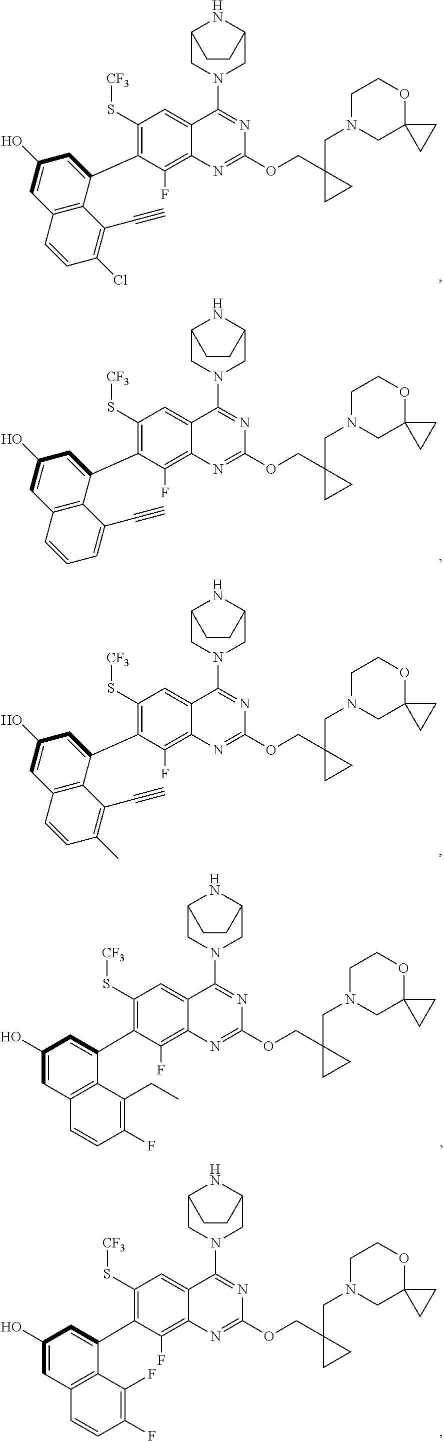

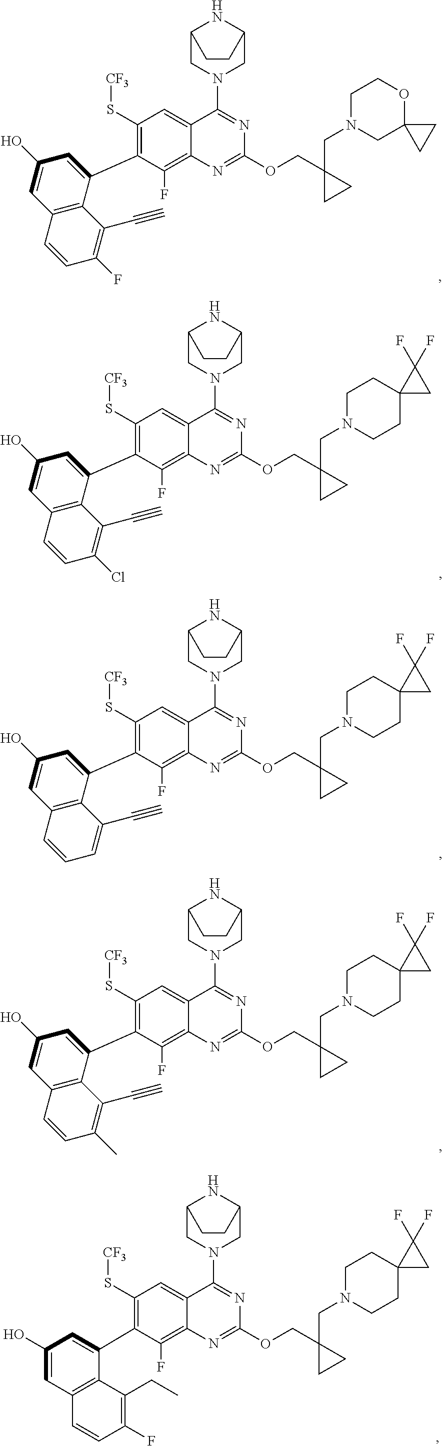

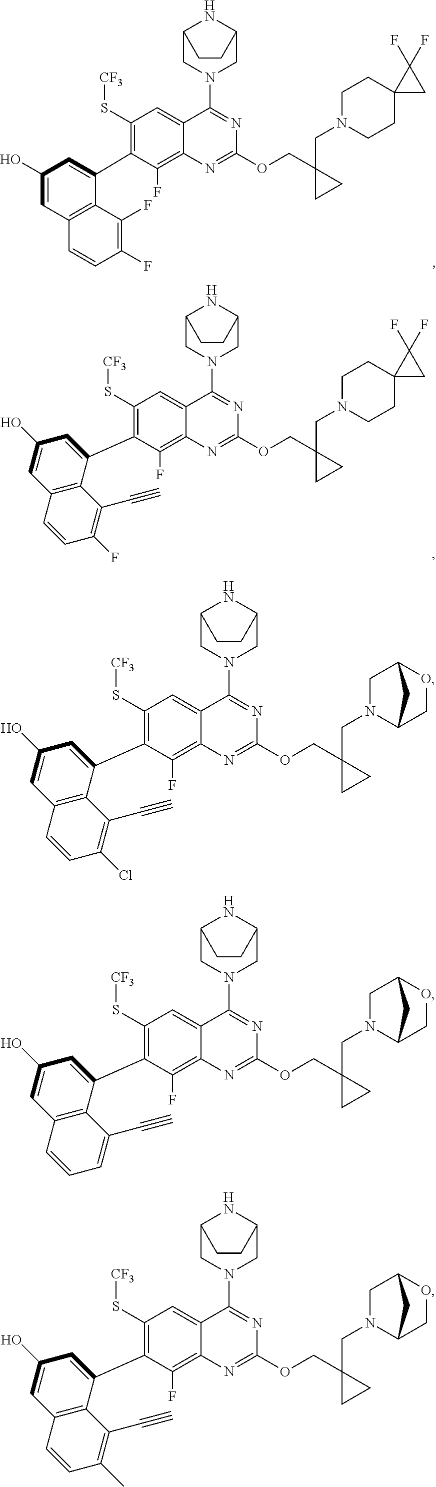

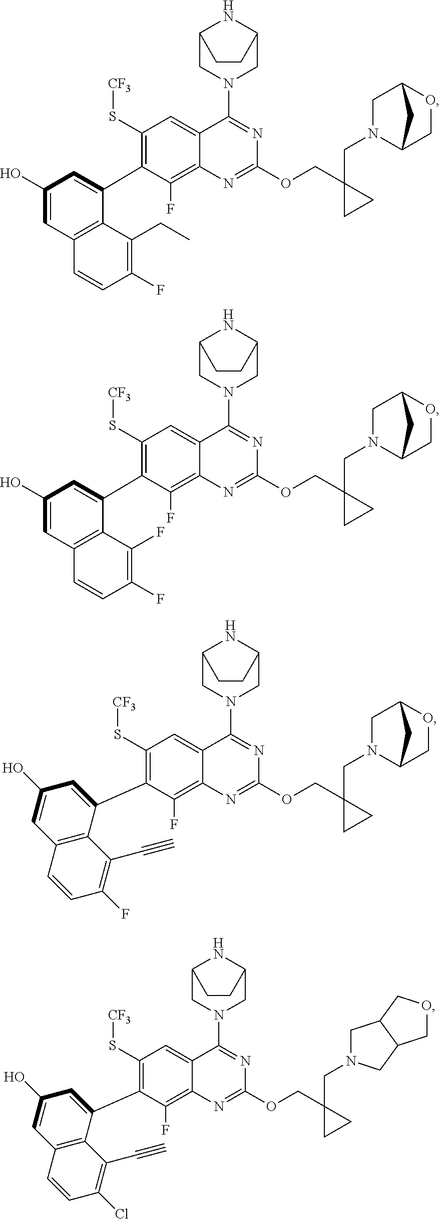

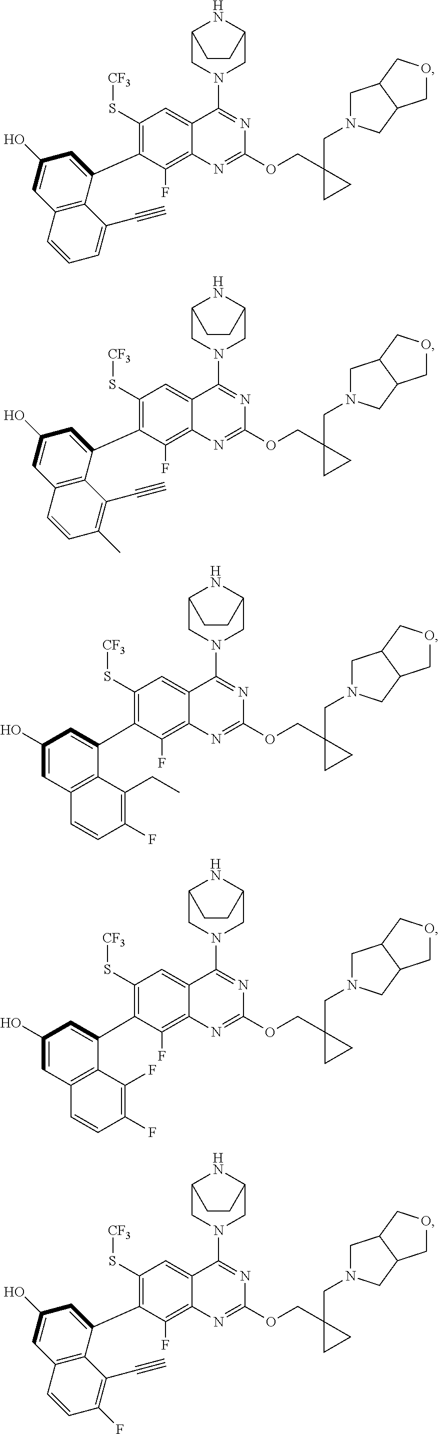

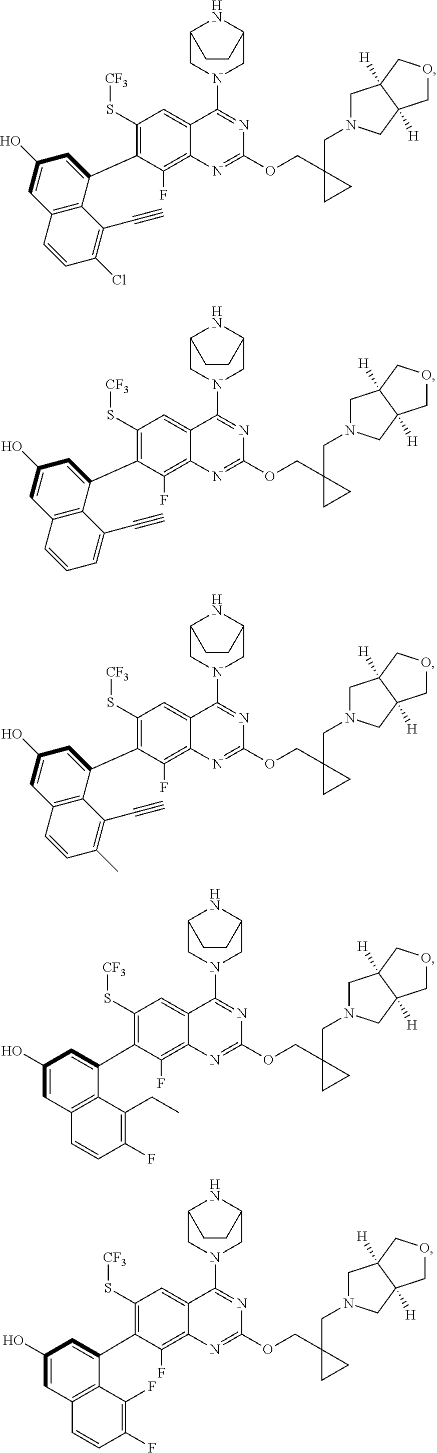

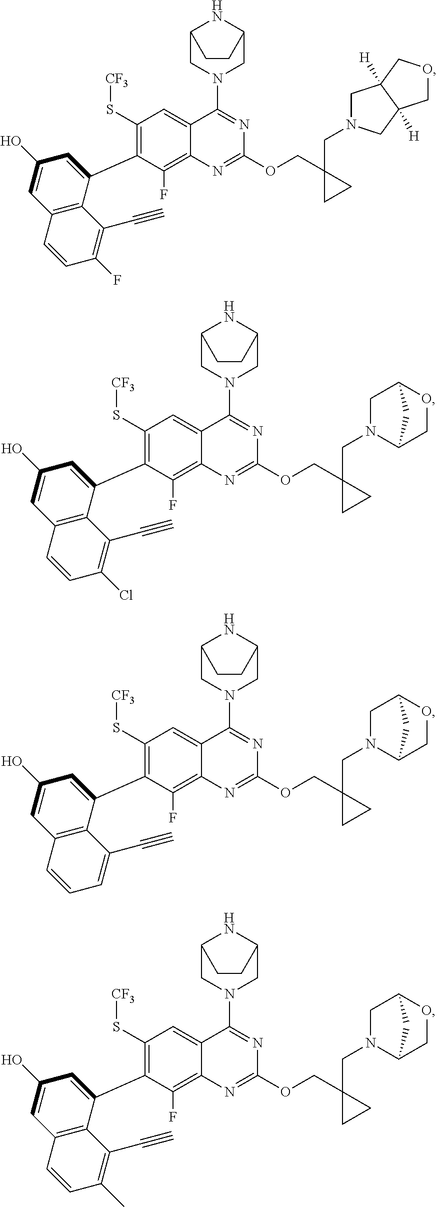

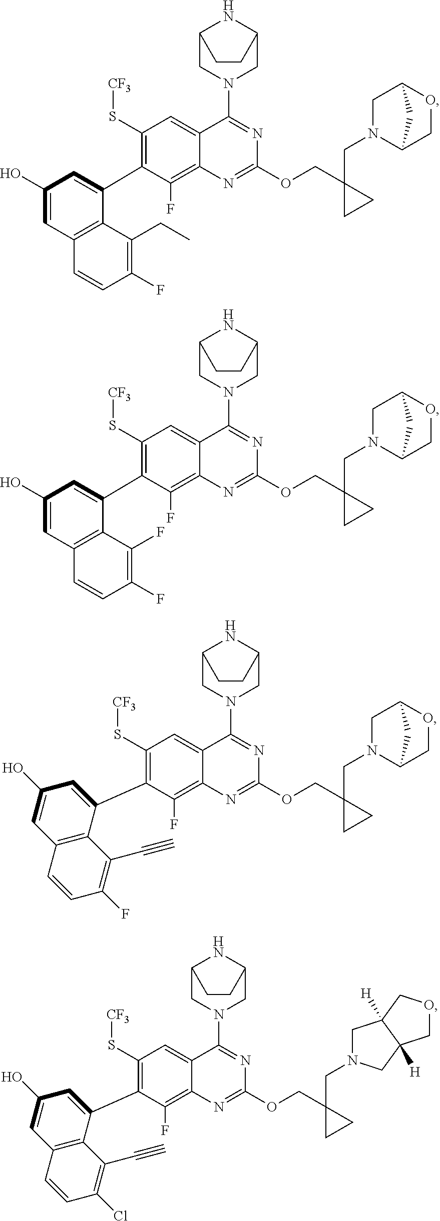

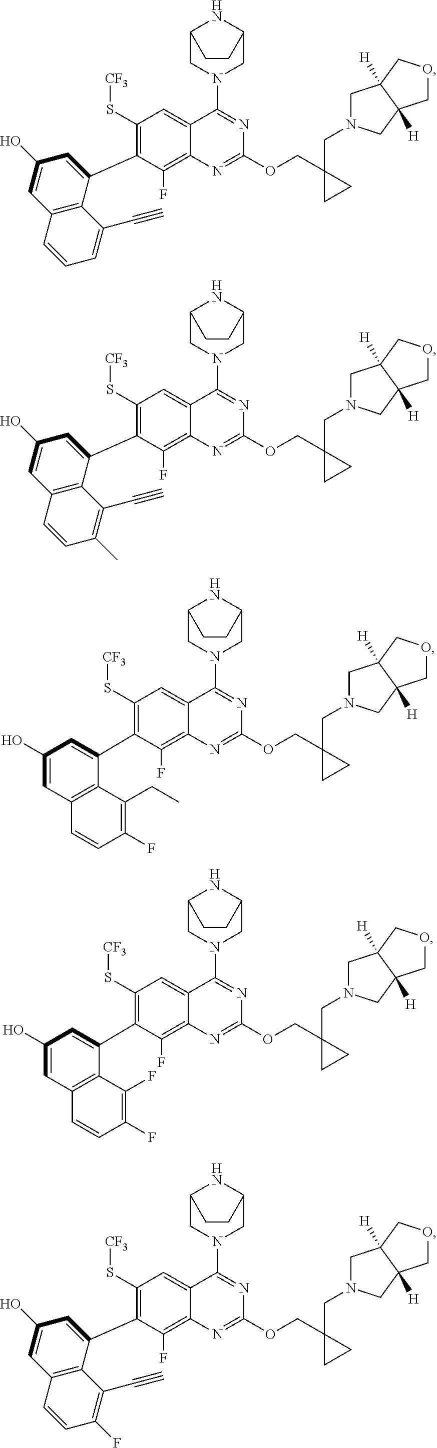

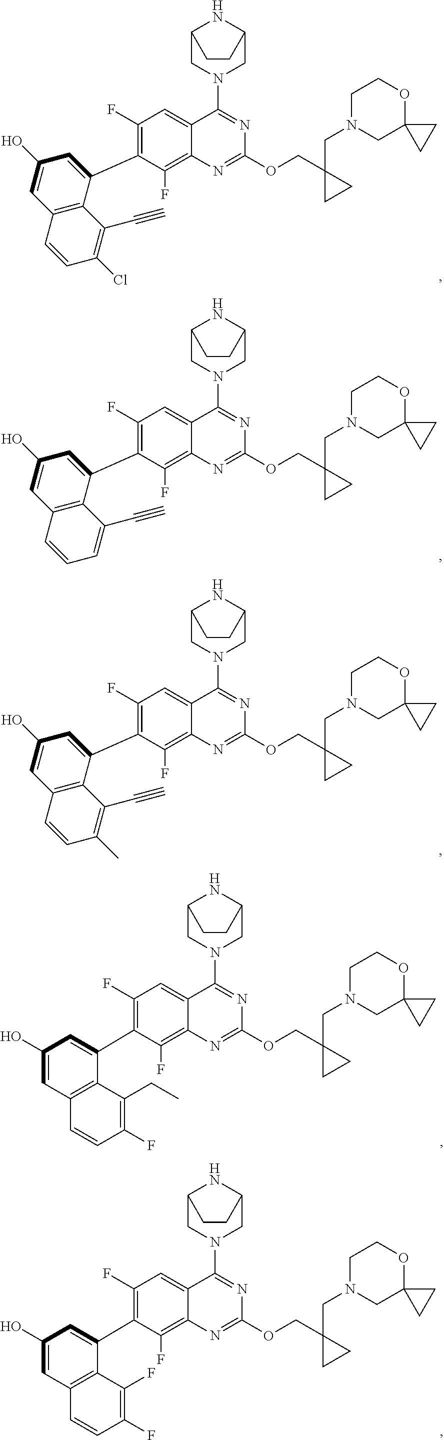

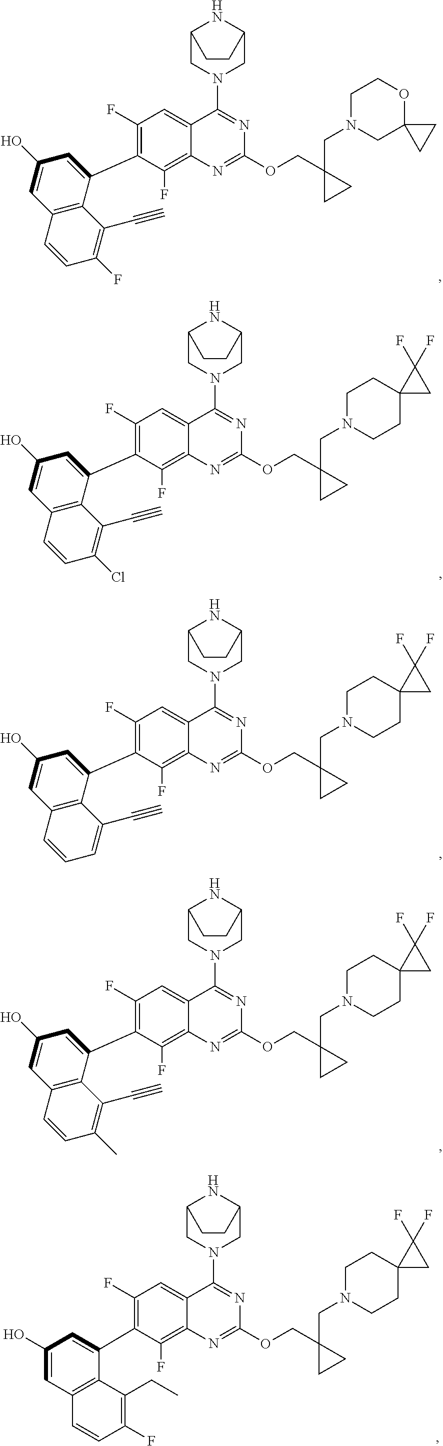

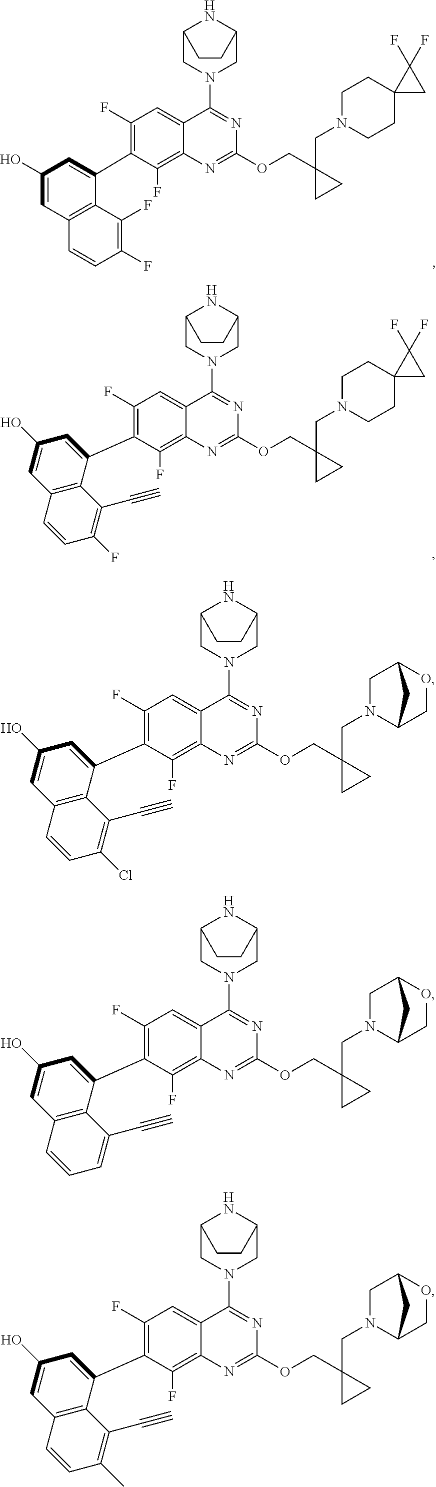

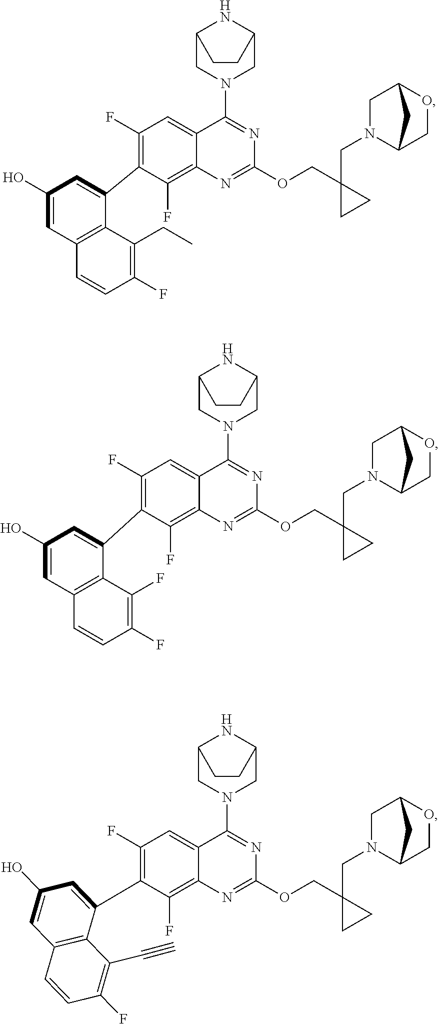

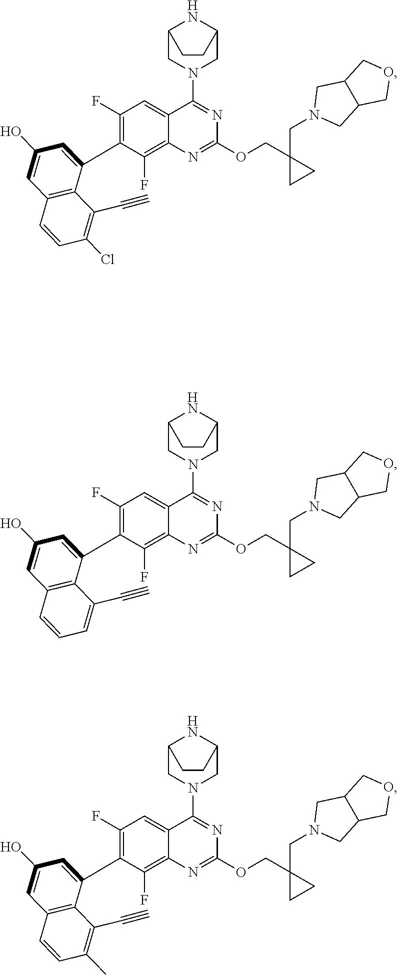

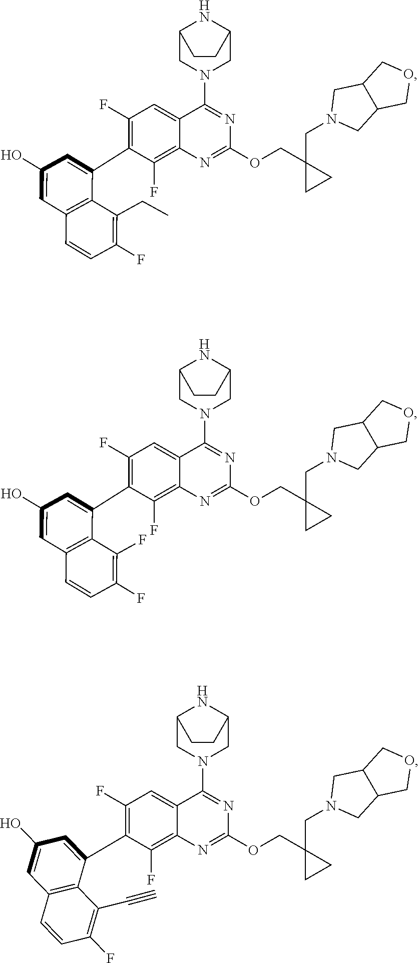

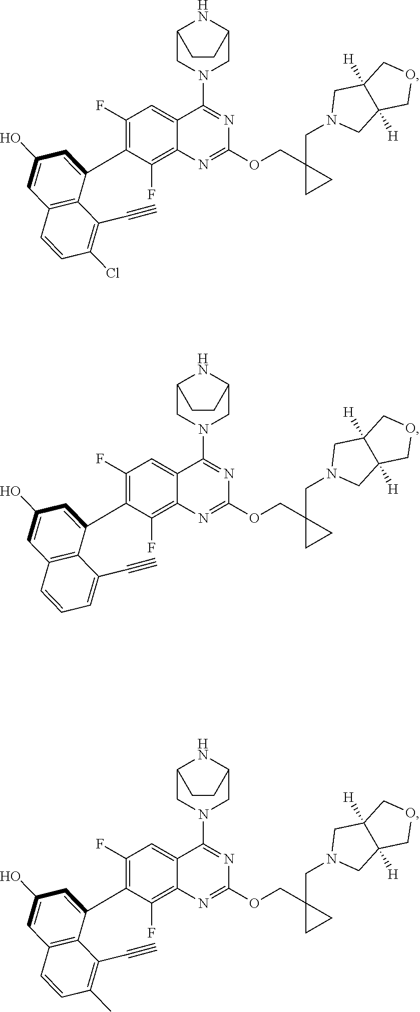

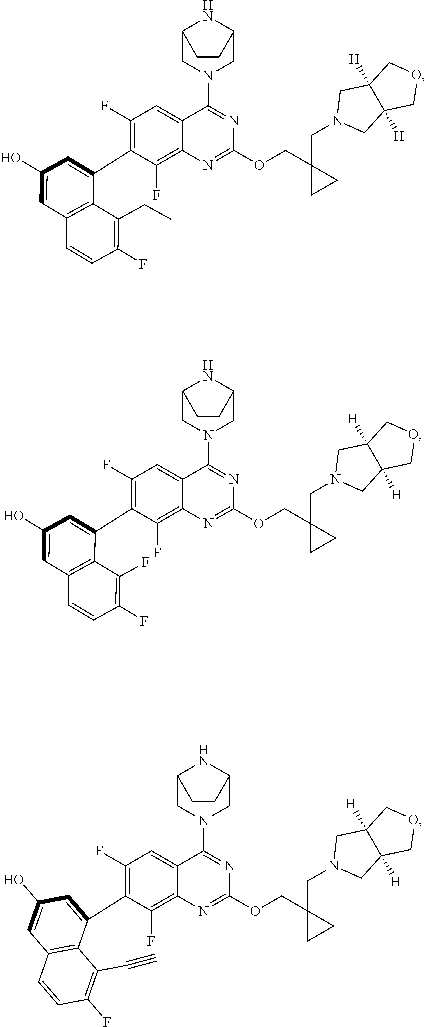

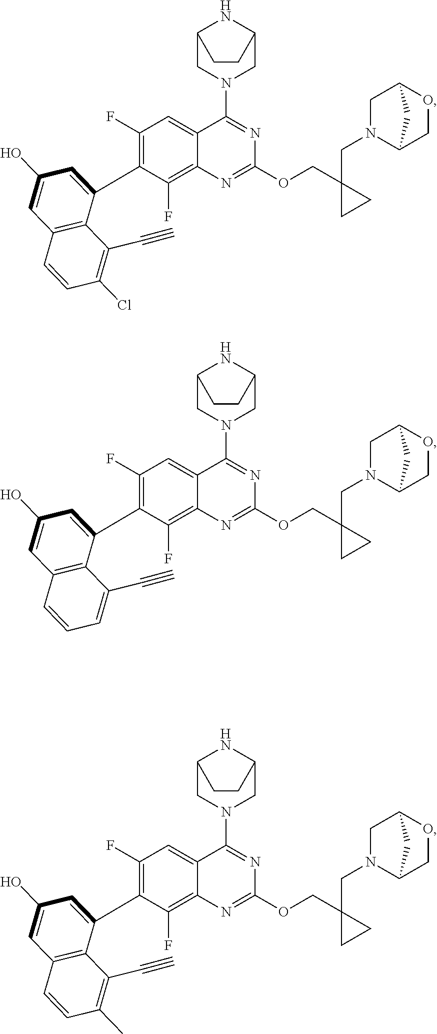

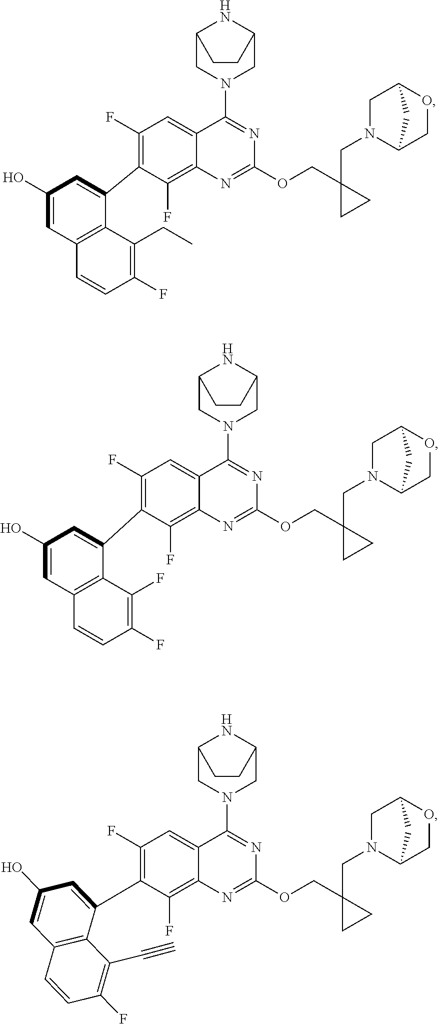

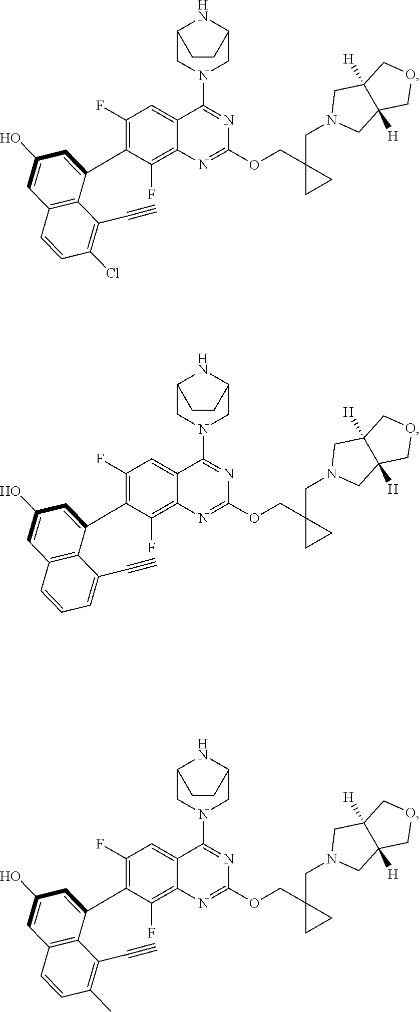

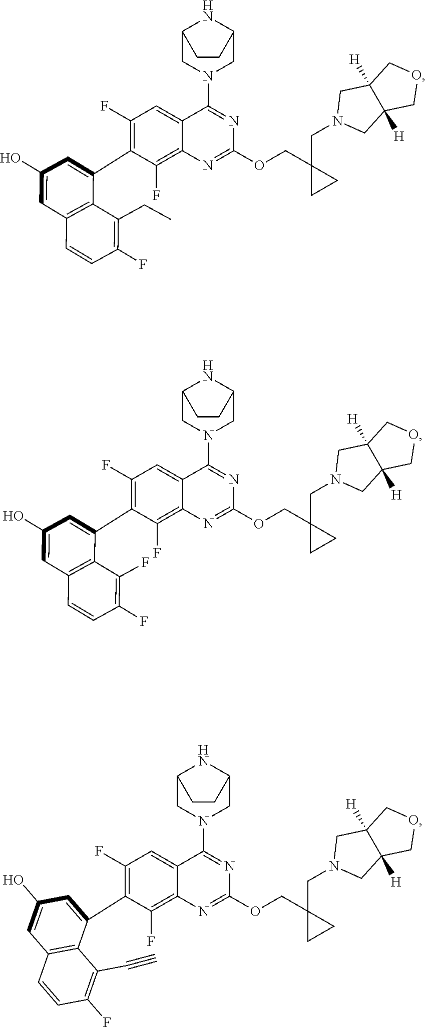

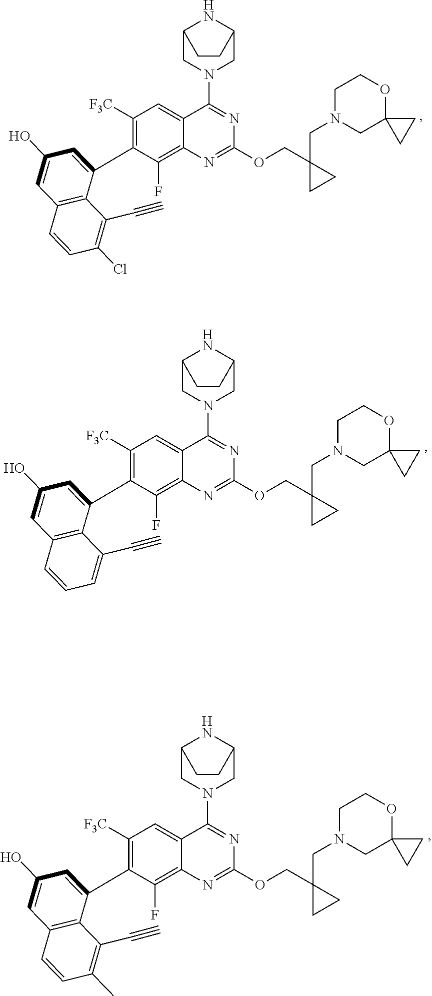

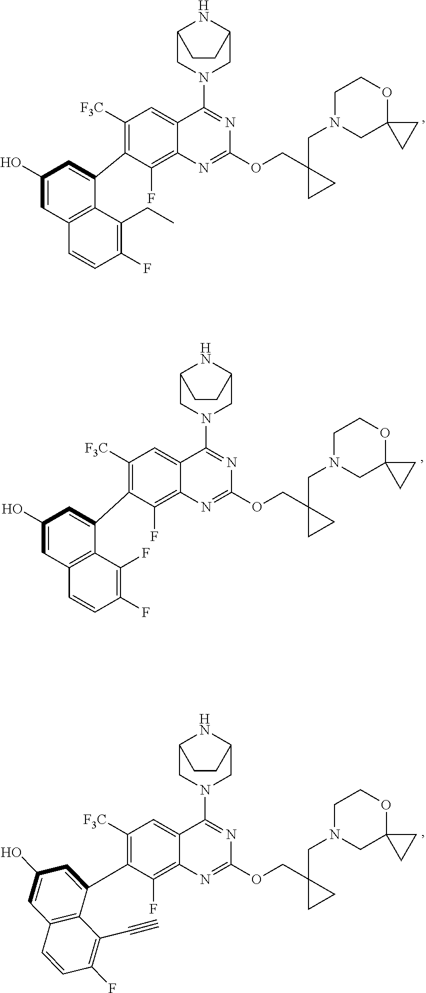

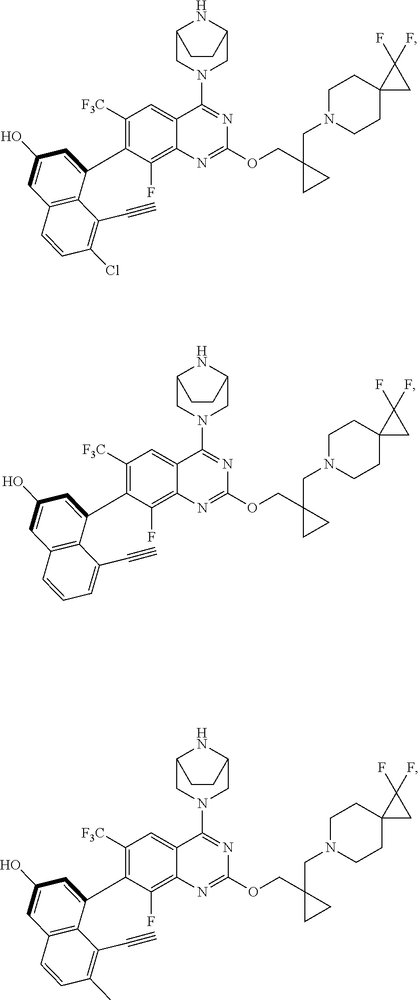

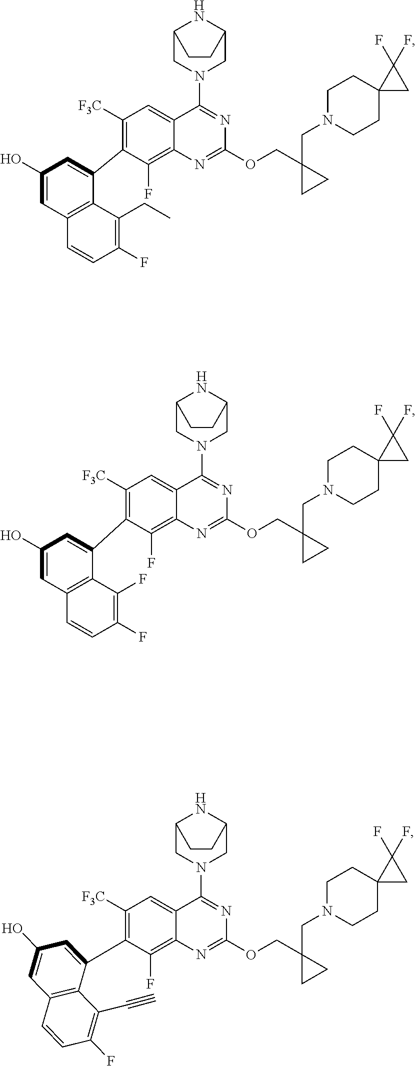

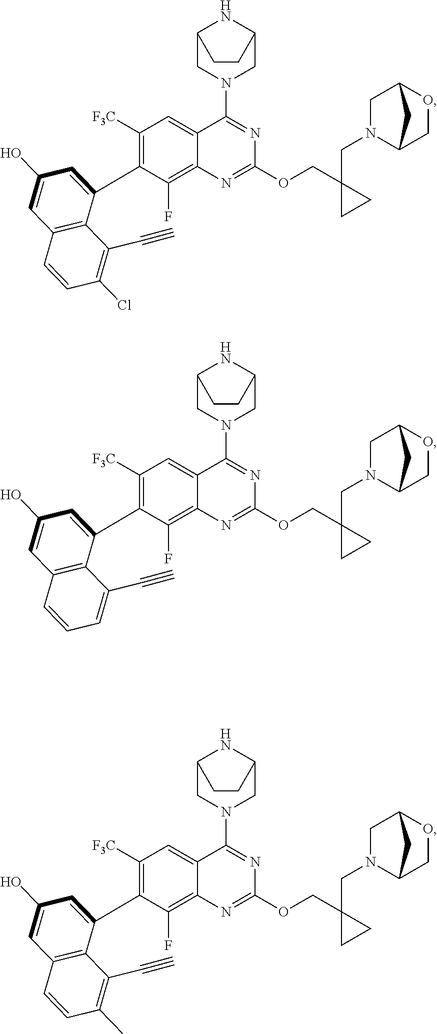

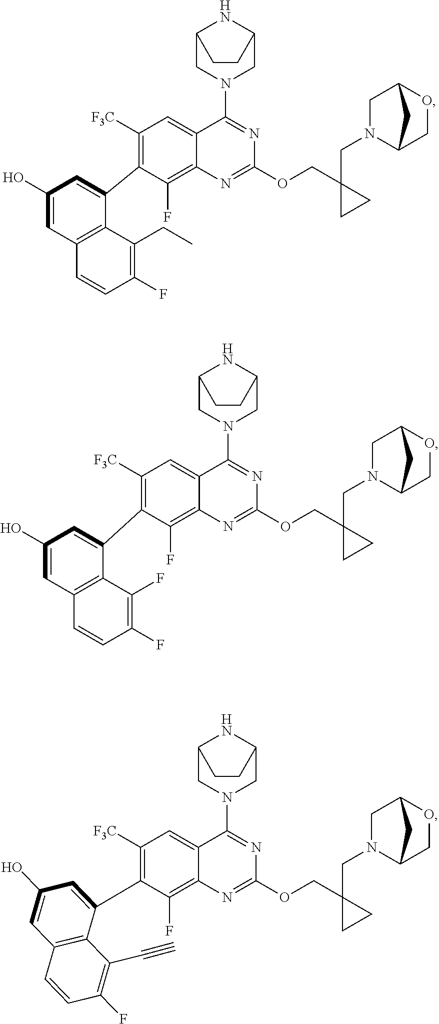

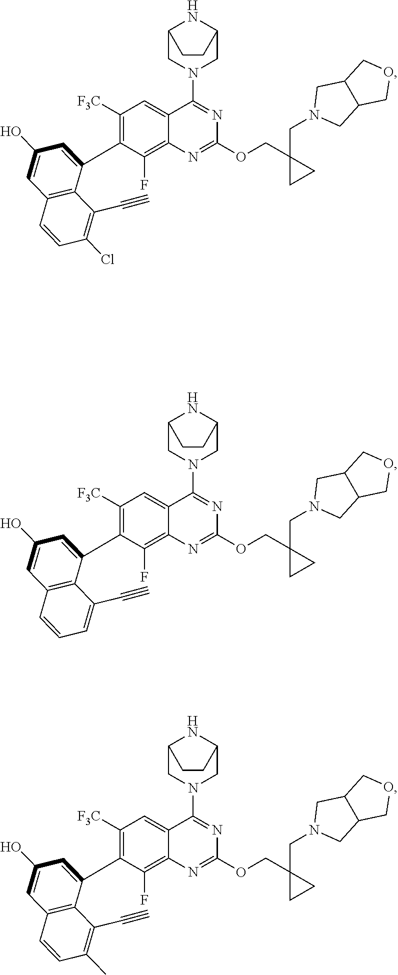

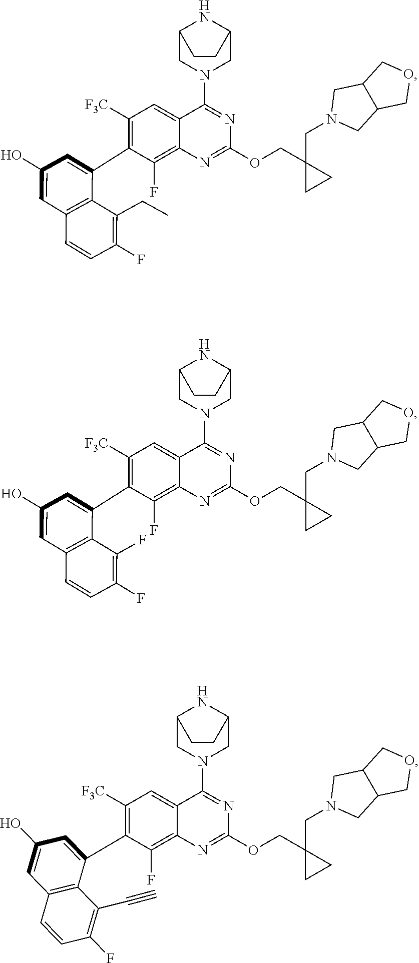

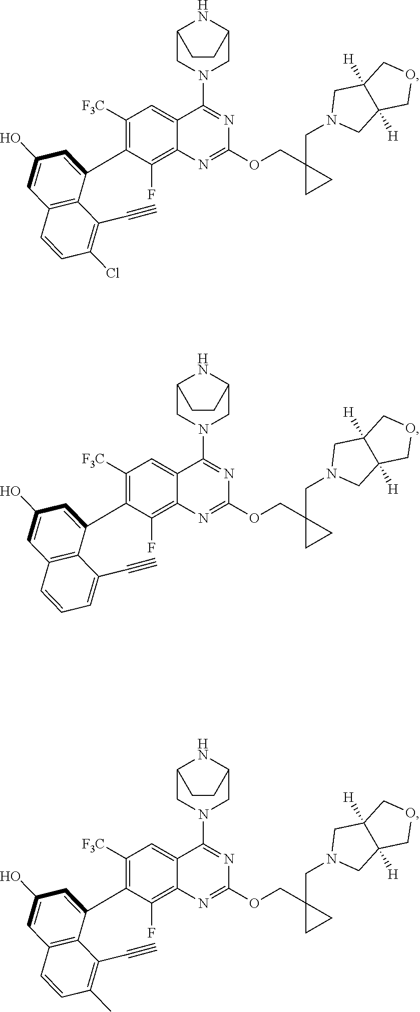

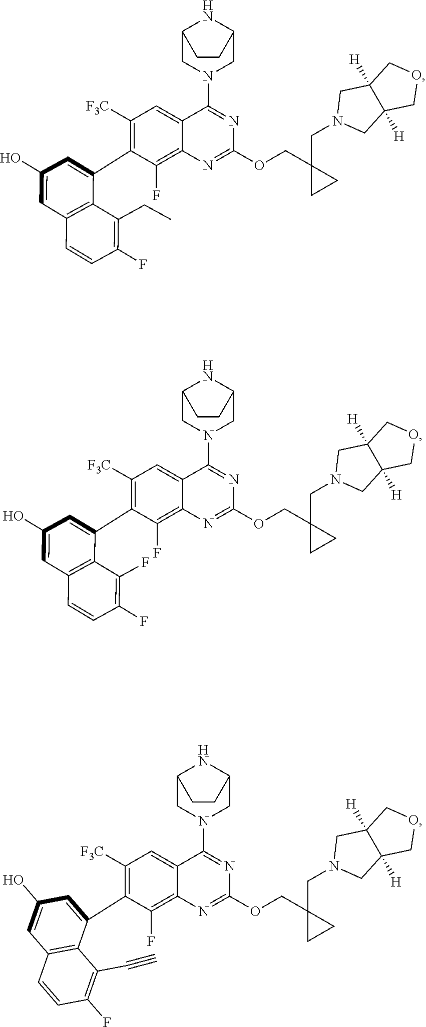

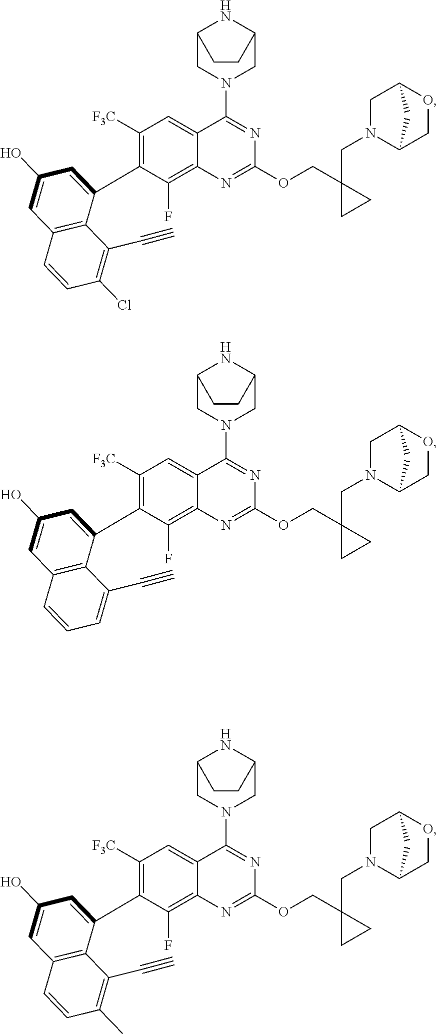

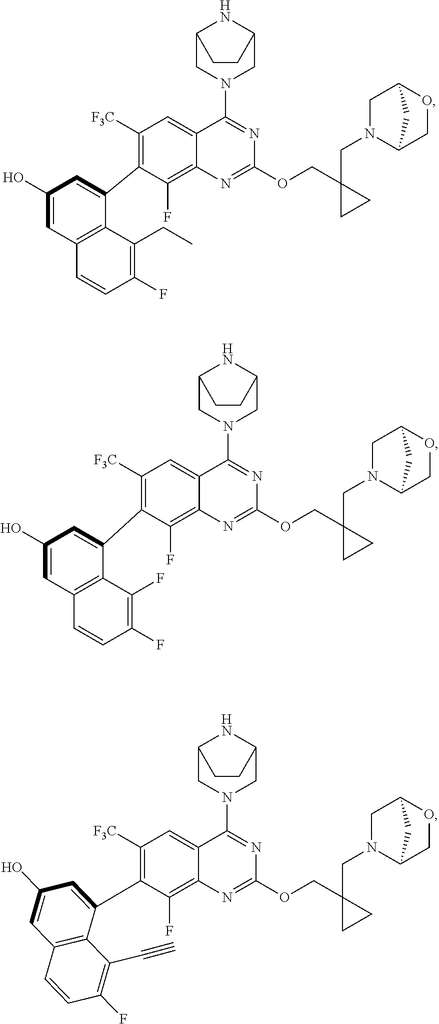

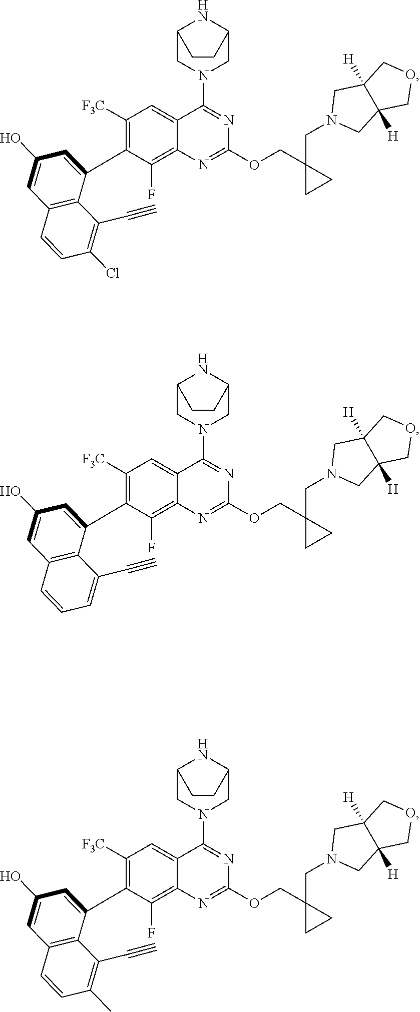

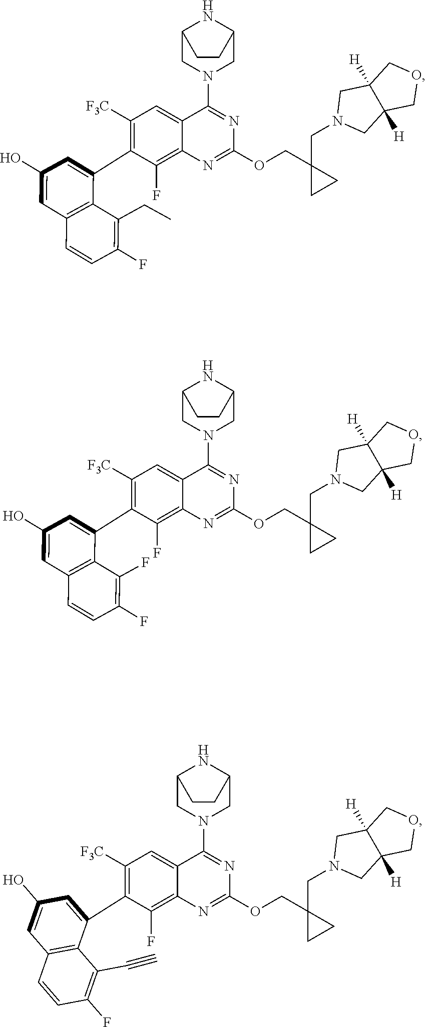

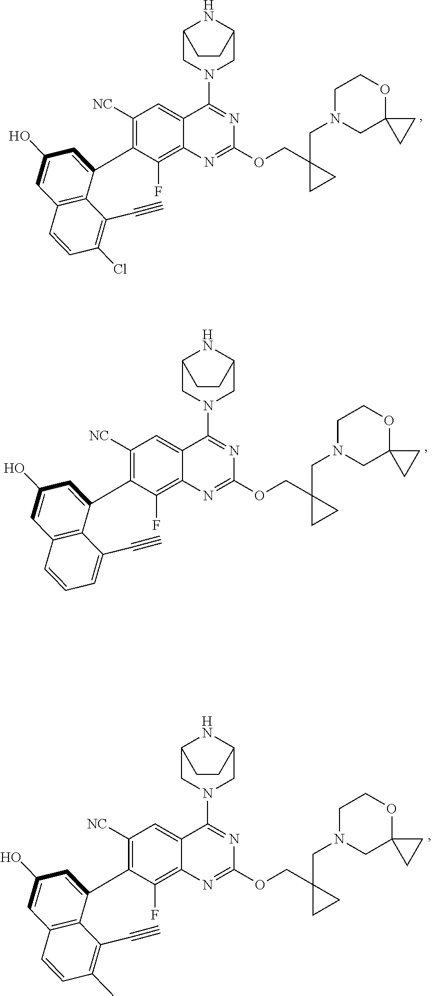

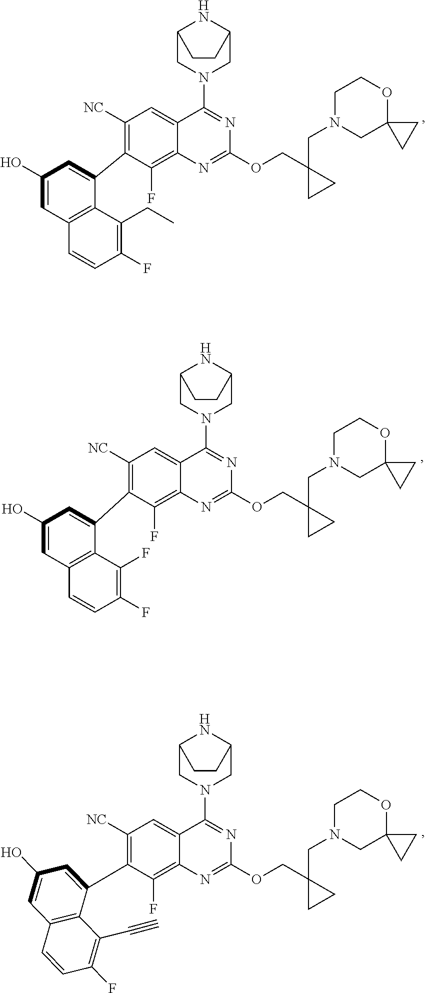

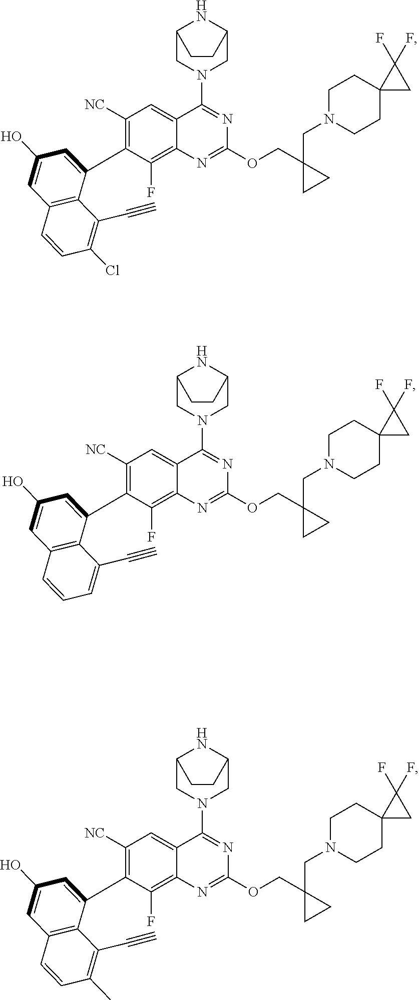

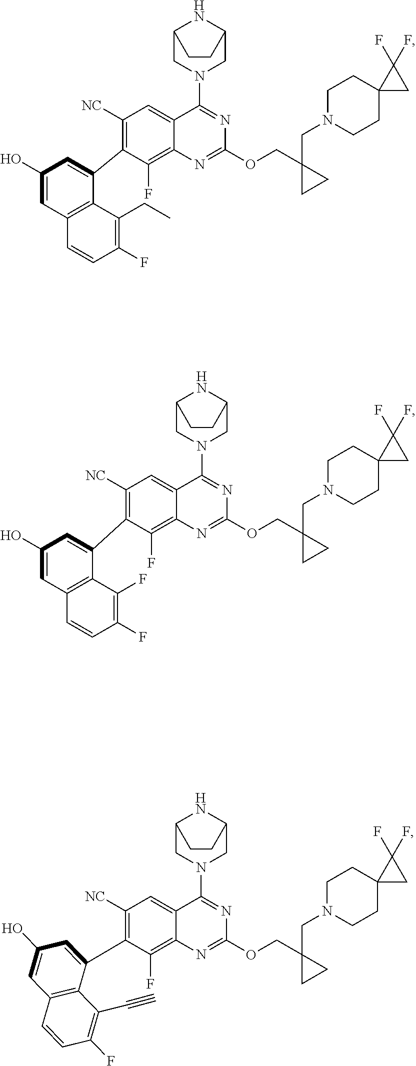

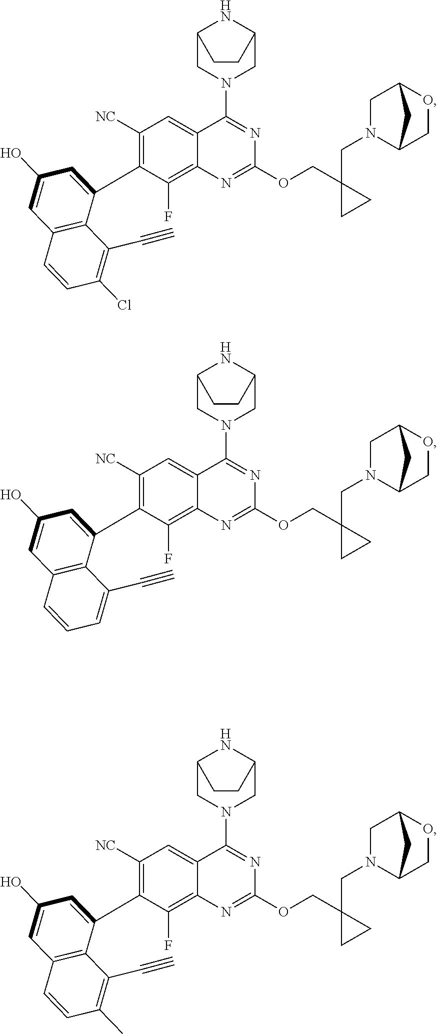

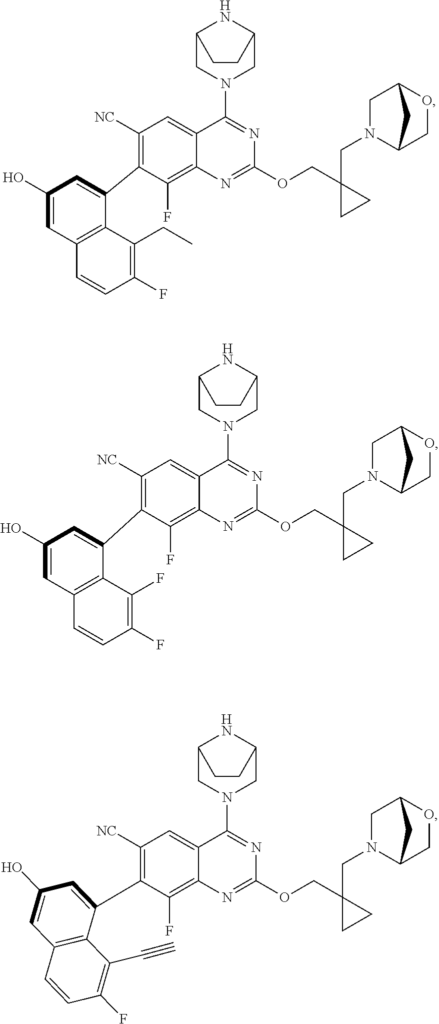

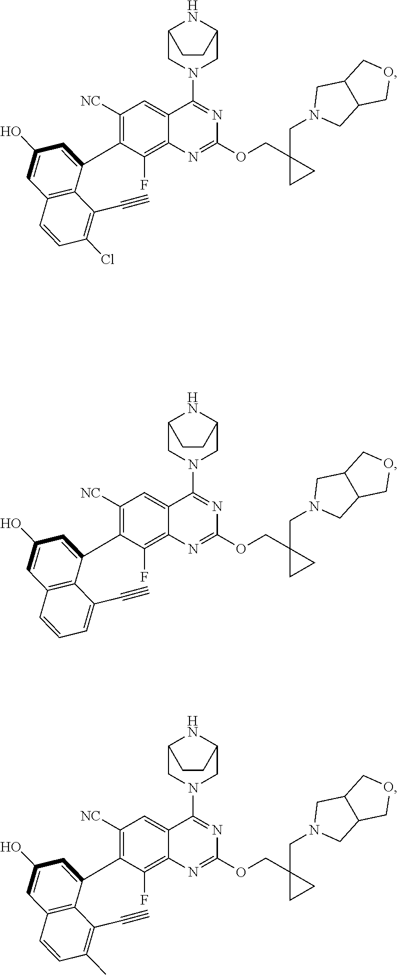

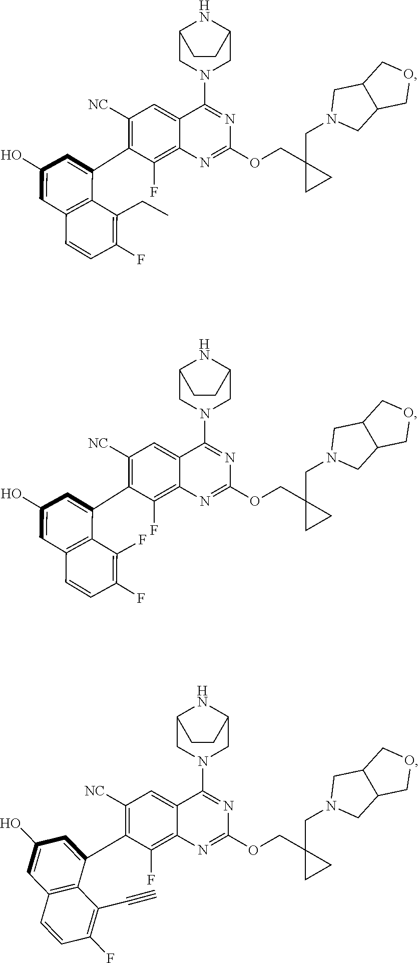

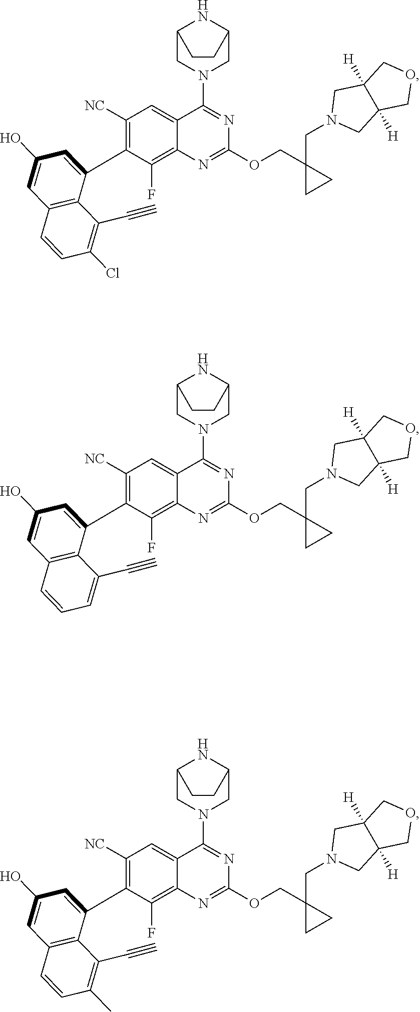

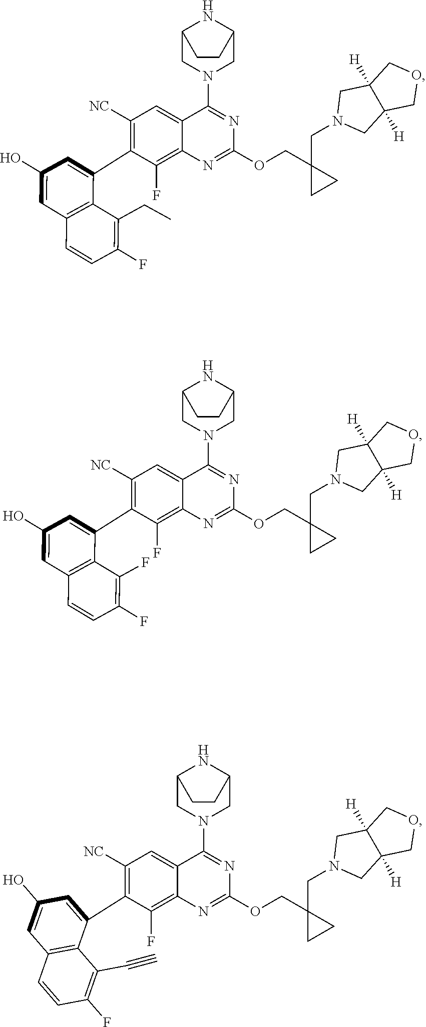

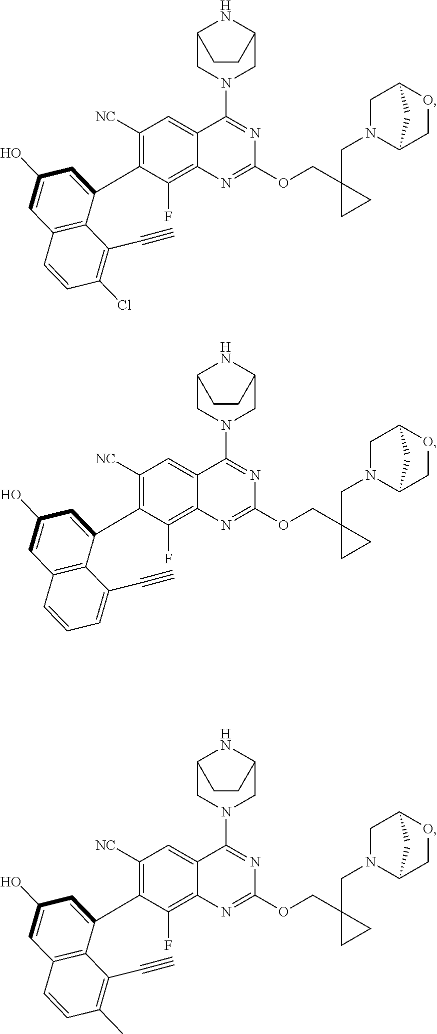

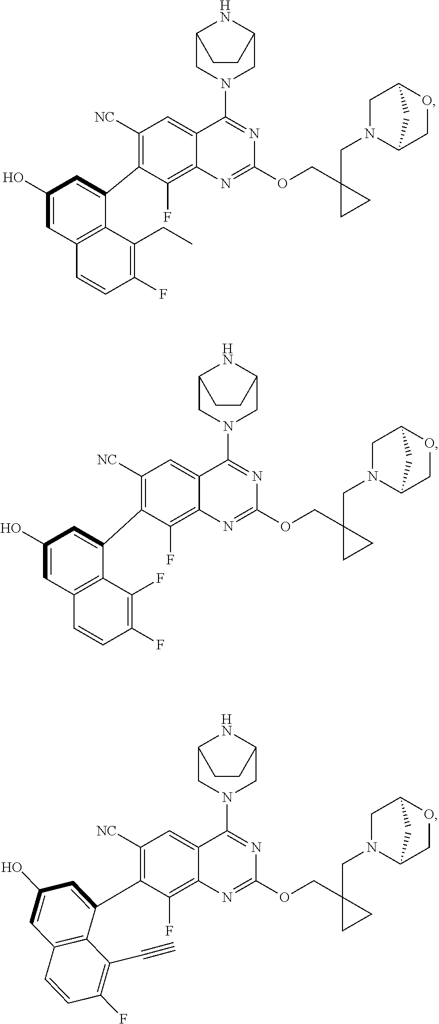

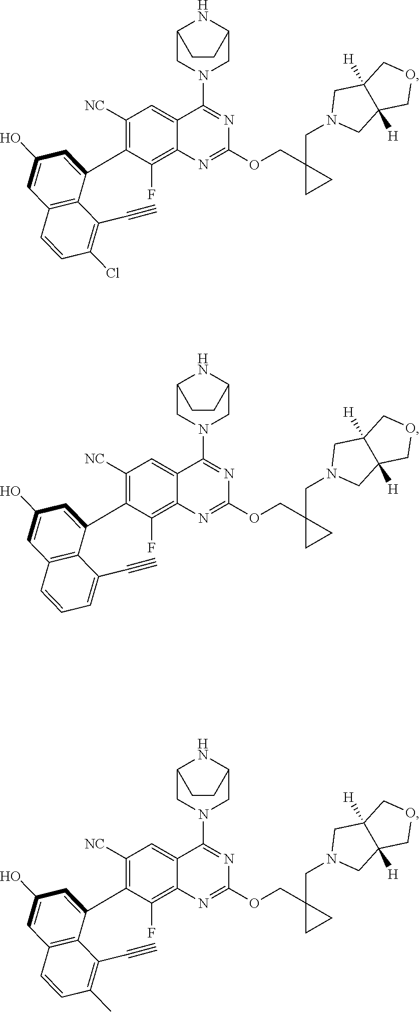

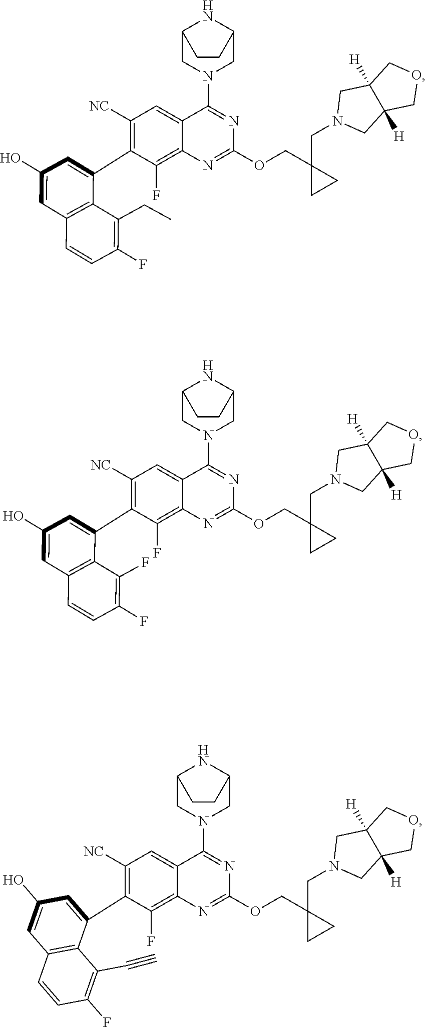

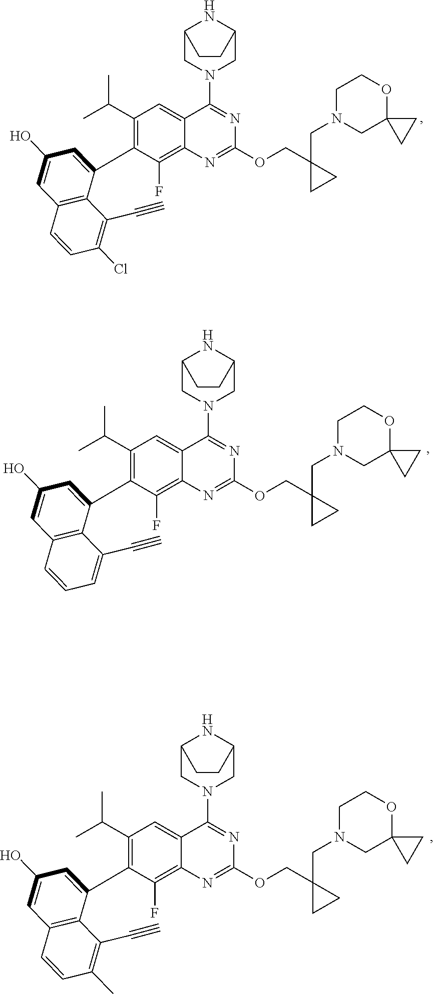

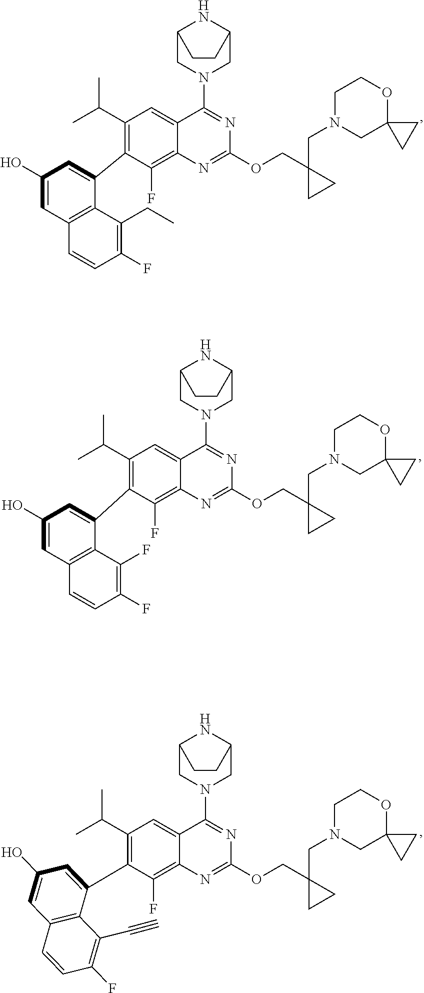

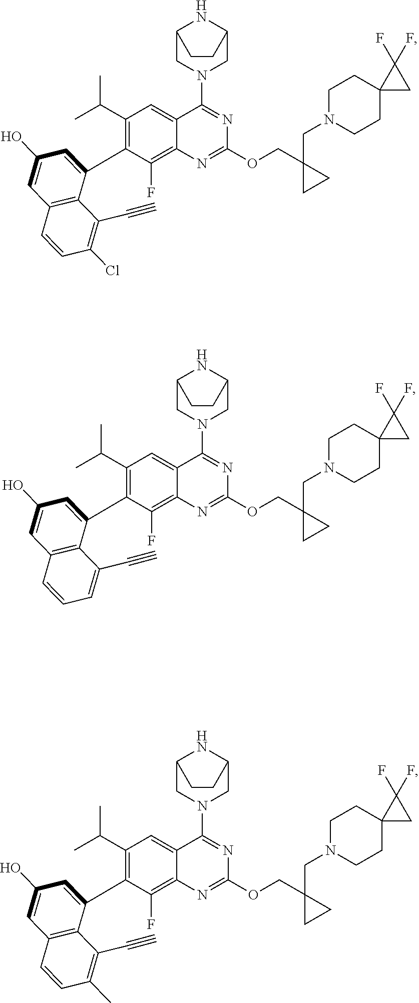

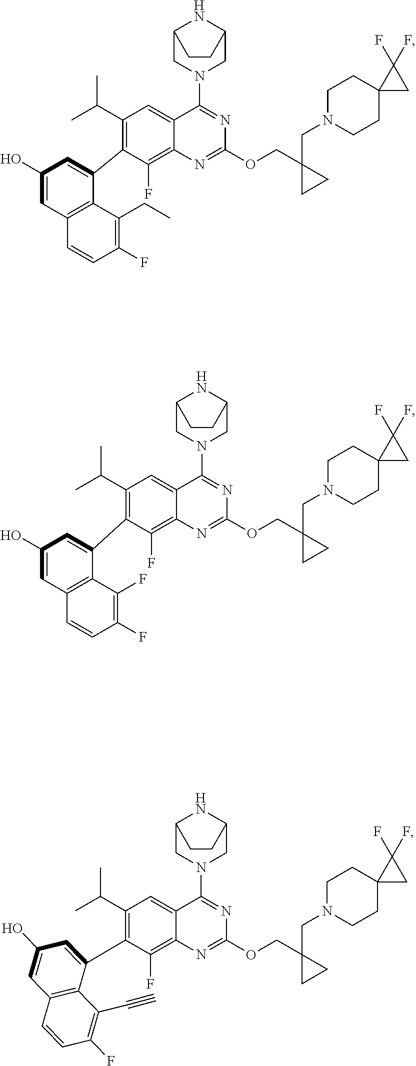

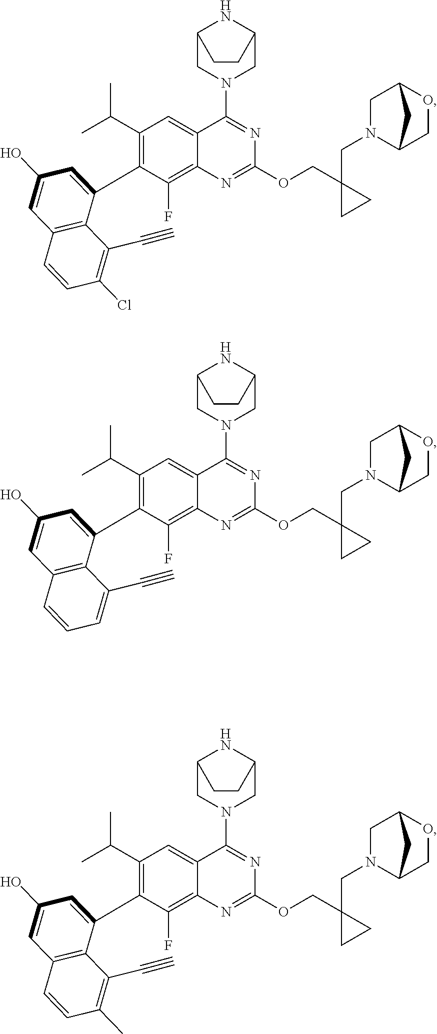

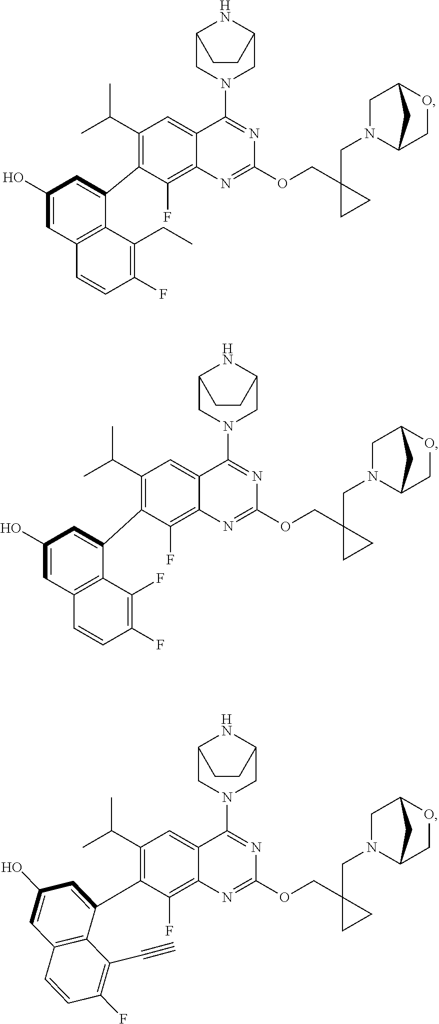

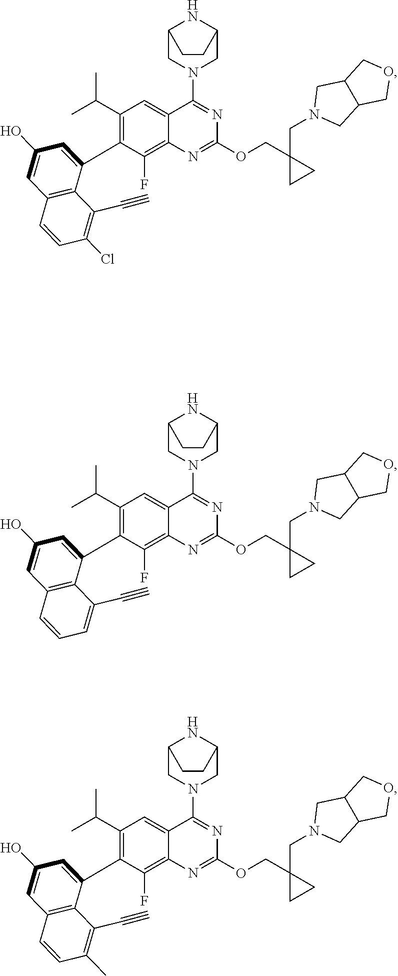

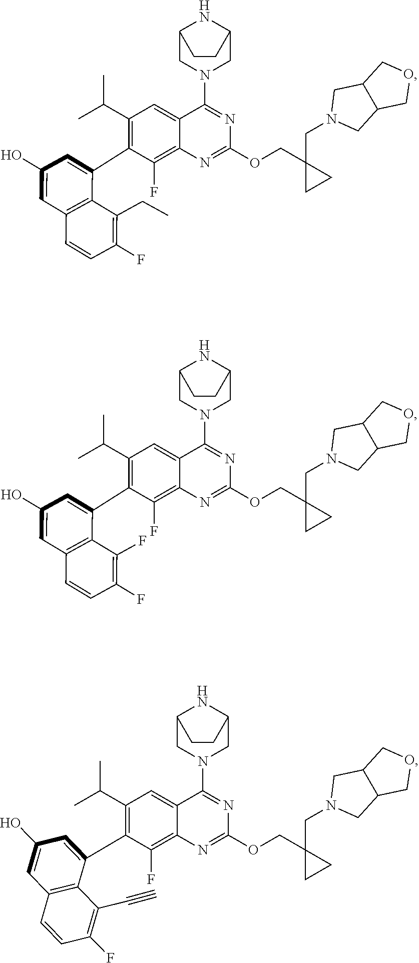

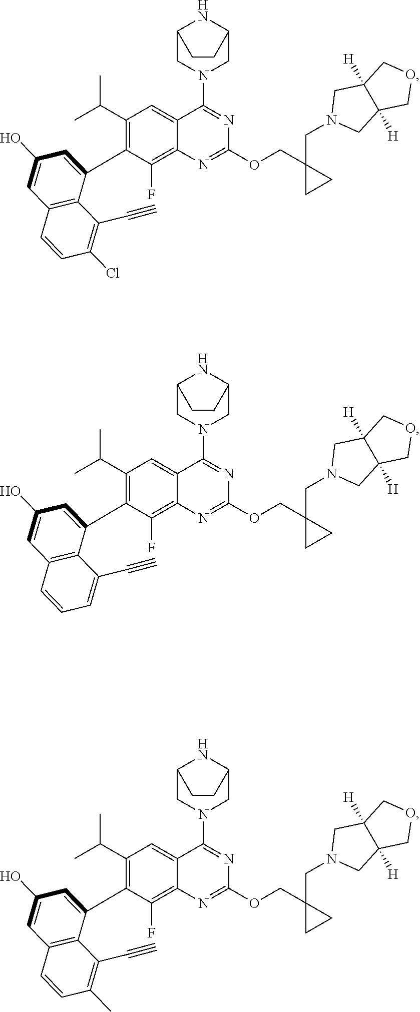

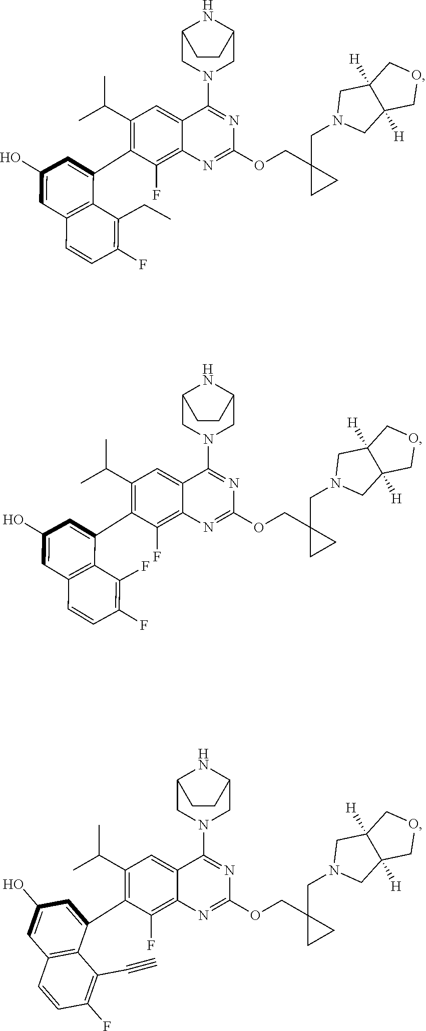

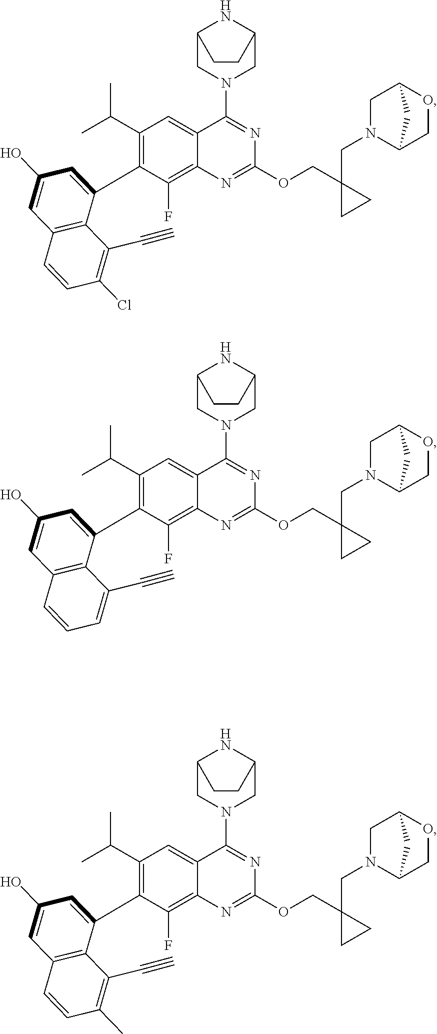

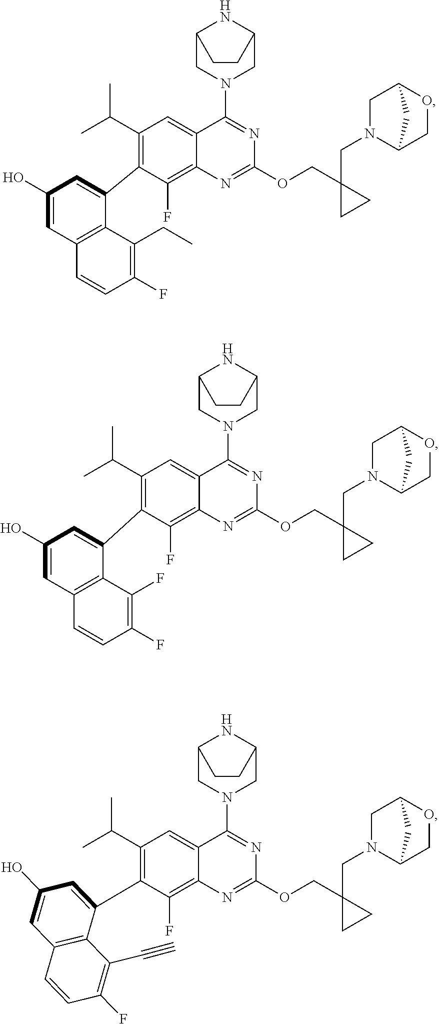

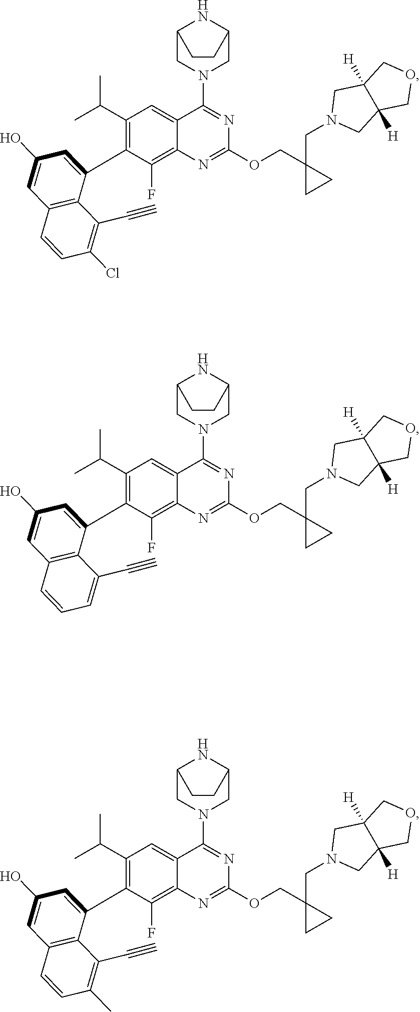

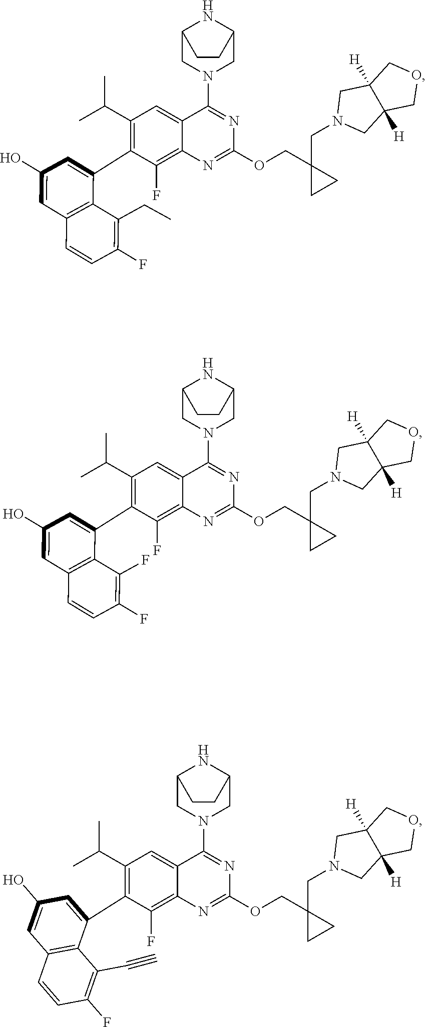

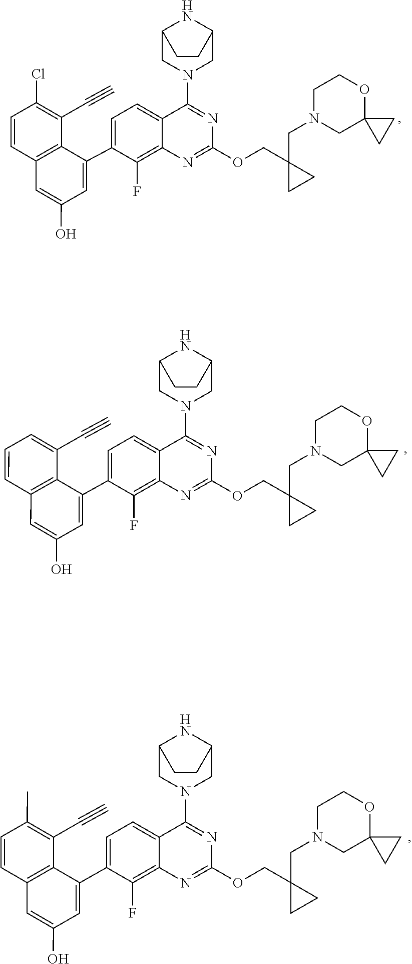 [SMILES: C#Cc1c(Cl)ccc2cc(O)cc(-c3ccc4c(N5CC6CCC(C5)N6)nc(OCC5(CN6CCOC7(CC7)C6)CC5)nc4c3F)c12 ; C#Cc1cccc2cc(O)cc(-c3ccc4c(N5CC6CCC(C5)N6)nc(OCC5(CN6CCOC7(CC7)C6)CC5)nc4c3F)c12 ; C#Cc1c(C)ccc2cc(O)cc(-c3ccc4c(N5CC6CCC(C5)N6)nc(OCC5(CN6CCOC7(CC7)C6)CC5)nc4c3F)c12]

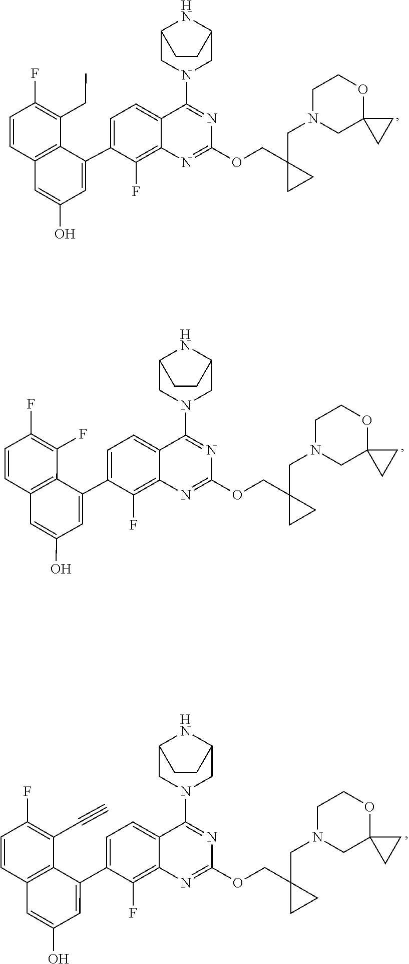 [SMILES: CCc1c(F)ccc2cc(O)cc(-c3ccc4c(N5CC6CCC(C5)N6)nc(OCC5(CN6CCOC7(CC7)C6)CC5)nc4c3F)c12 ; Oc1cc(-c2ccc3c(N4CC5CCC(C4)N5)nc(OCC4(CN5CCOC6(CC6)C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CCOC7(CC7)C6)CC5)nc4c3F)c12]

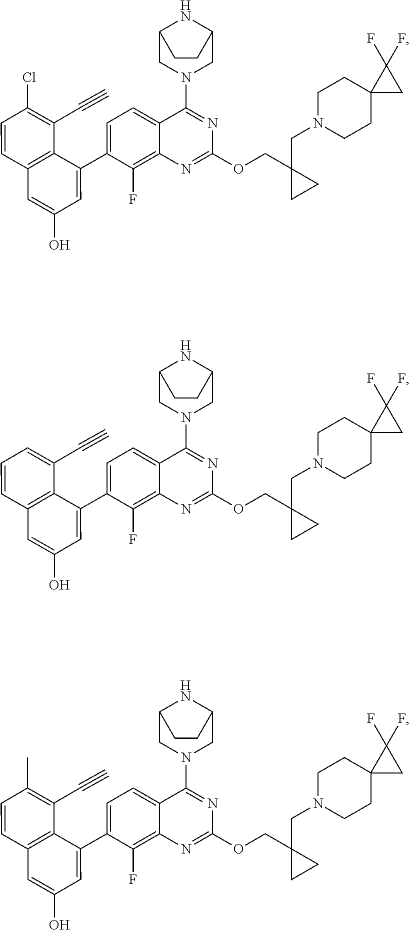 [SMILES: C#Cc1c(Cl)ccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; C#Cc1c(C)ccc2cc(O)cc(-c3ccc4c(N5CC6CCC(C5)N6)nc(OCC5(CN6CCC7(CC6)CC7(F)F)CC5)nc4c3F)c12]

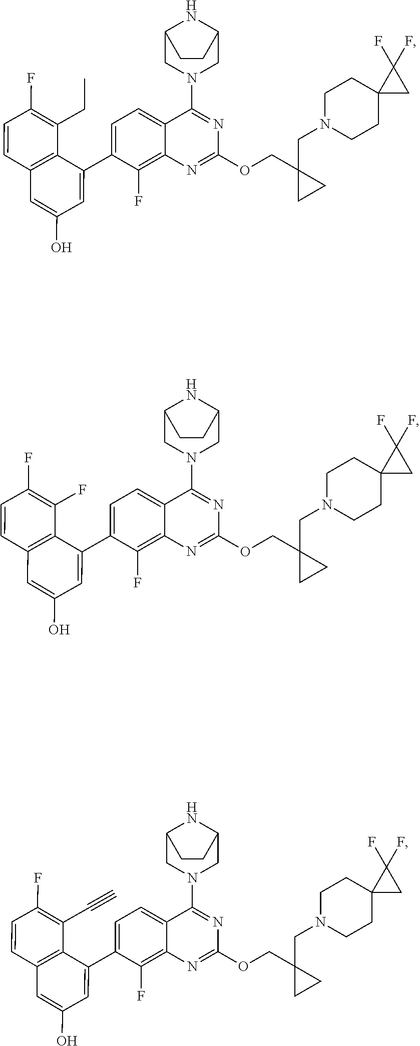 [SMILES: CCc1c(F)ccc2cc(O)cc(-c3ccc4c(N5CC6CCC(C5)N6)nc(OCC5(CN6CCC7(CC6)CC7(F)F)CC5)nc4c3F)c12 ; Oc1cc(-c2ccc3c(N4CC5CCC(C4)N5)nc(OCC4(CN5CCC6(CC5)CC6(F)F)CC4)nc3c2F)c2c(F)c(F)ccc2c1 ; C#Cc1c(F)ccc2cc(O)cc(-c3ccc4c(N5CC6CCC(C5)N6)nc(OCC5(CN6CCC7(CC6)CC7(F)F)CC5)nc4c3F)c12]

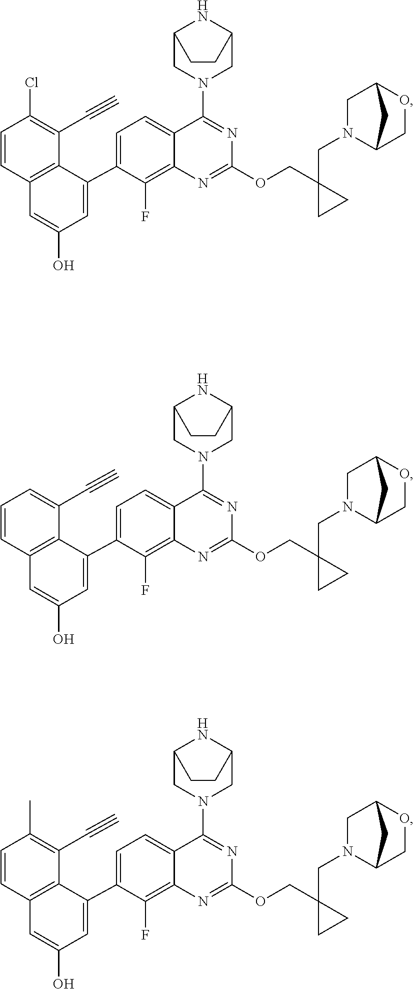 [SMILES: C#Cc1c(Cl)ccc2cc(O)cc(-c3ccc4c(N5CC6CCC(C5)N6)nc(OCC5(CN6C[C@H]7C[C@@H]6CO7)CC5)nc4c3F)c12 ; C#Cc1cccc2cc(O)cc(-c3ccc4c(N5CC6CCC(C5)N6)nc(OCC5(CN6C[C@H]7C[C@@H]6CO7)CC5)nc4c3F)c12 ; C#Cc1c(C)ccc2cc(O)cc(-c3ccc4c(N5CC6CCC(C5)N6)nc(OCC5(CN6C[C@H]7C[C@@H]6CO7)CC5)nc4c3F)c12]

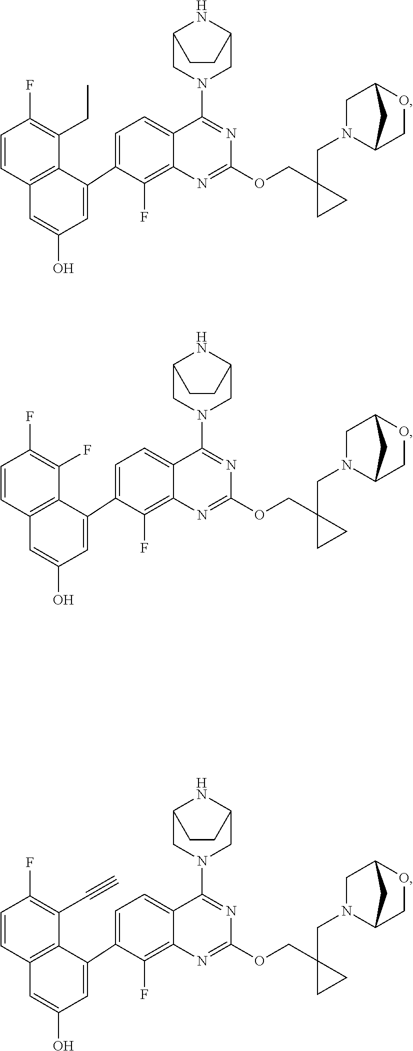 [SMILES: CCc1c(F)ccc2cc(O)cc(-c3ccc4c(N5CC6CCC(C5)N6)nc(OCC5(CN6C[C@H]7C[C@@H]6CO7)CC5)nc4c3F)c12 ; Oc1cc(-c2ccc3c(N4CC5CCC(C4)N5)nc(OCC4(CN5C[C@H]6C[C@@H]5CO6)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7C[C@@H]6CO7)CC5)nc4c3F)c12]

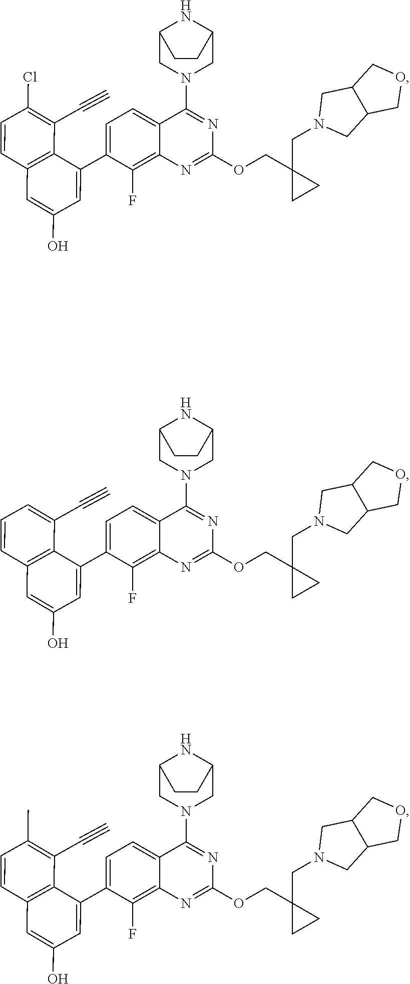 [SMILES: C#Cc1c(Cl)ccc2cc(O)cc(-c3ccc4c(N5CC6CCC(C5)N6)nc(OCC5(CN6CC7COCC7C6)CC5)nc4c3F)c12 ; C#Cc1cccc2cc(O)cc(-c3ccc4c(N5CC6CCC(C5)N6)nc(OCC5(CN6CC7COCC7C6)CC5)nc4c3F)c12 ; C#Cc1c(C)ccc2cc(O)cc(-c3ccc4c(N5CC6CCC(C5)N6)nc(OCC5(CN6CC7COCC7C6)CC5)nc4c3F)c12]

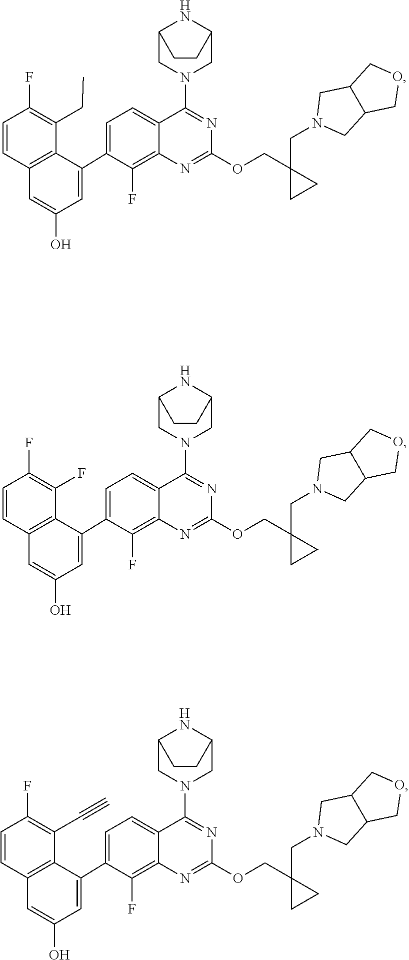 [SMILES: CCc1c(F)ccc2cc(O)cc(-c3ccc4c(N5CC6CCC(C5)N6)nc(OCC5(CN6CC7COCC7C6)CC5)nc4c3F)c12 ; Oc1cc(-c2ccc3c(N4CC5CCC(C4)N5)nc(OCC4(CN5CC6COCC6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7COCC7C6)CC5)nc4c3F)c12]

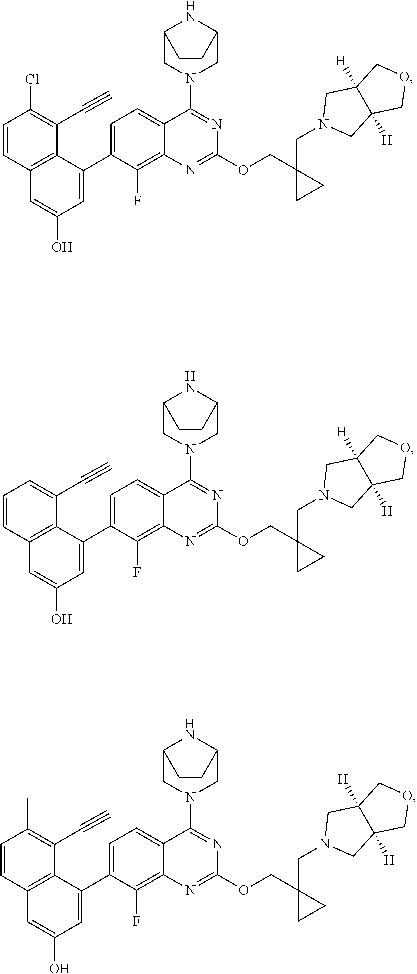 [SMILES: C#Cc1c(Cl)ccc2cc(O)cc(-c3ccc4c(N5CC6CCC(C5)N6)nc(OCC5(CN6C[C@H]7COC[C@H]7C6)CC5)nc4c3F)c12 ; C#Cc1cccc2cc(O)cc(-c3ccc4c(N5CC6CCC(C5)N6)nc(OCC5(CN6C[C@H]7COC[C@H]7C6)CC5)nc4c3F)c12 ; C#Cc1c(C)ccc2cc(O)cc(-c3ccc4c(N5CC6CCC(C5)N6)nc(OCC5(CN6C[C@H]7COC[C@H]7C6)CC5)nc4c3F)c12]

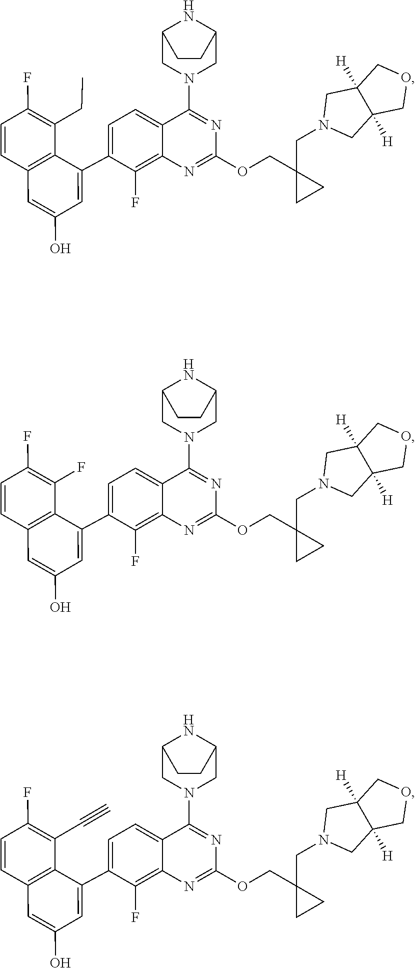 [SMILES: CCc1c(F)ccc2cc(O)cc(-c3ccc4c(N5CC6CCC(C5)N6)nc(OCC5(CN6C[C@H]7COC[C@H]7C6)CC5)nc4c3F)c12 ; Oc1cc(-c2ccc3c(N4CC5CCC(C4)N5)nc(OCC4(CN5C[C@H]6COC[C@H]6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7COC[C@H]7C6)CC5)nc4c3F)c12]

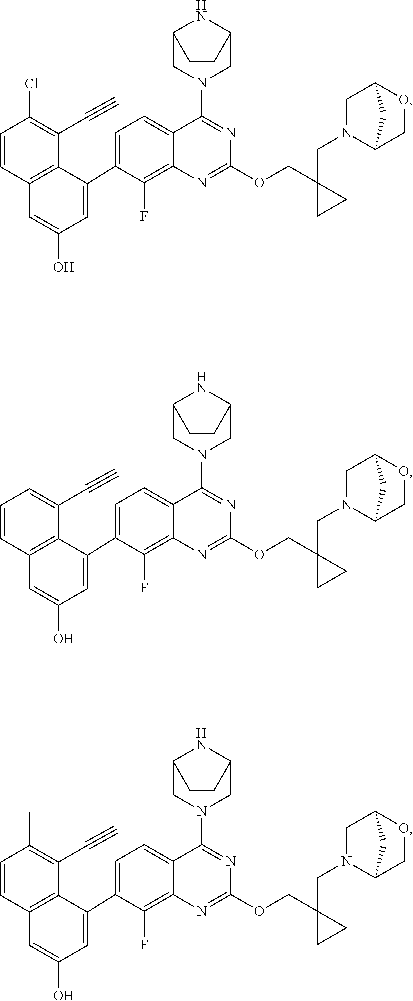 [SMILES: C#Cc1c(Cl)ccc2cc(O)cc(-c3ccc4c(N5CC6CCC(C5)N6)nc(OCC5(CN6C[C@@H]7C[C@H]6CO7)CC5)nc4c3F)c12 ; C#Cc1cccc2cc(O)cc(-c3ccc4c(N5CC6CCC(C5)N6)nc(OCC5(CN6C[C@@H]7C[C@H]6CO7)CC5)nc4c3F)c12 ; C#Cc1c(C)ccc2cc(O)cc(-c3ccc4c(N5CC6CCC(C5)N6)nc(OCC5(CN6C[C@@H]7C[C@H]6CO7)CC5)nc4c3F)c12]

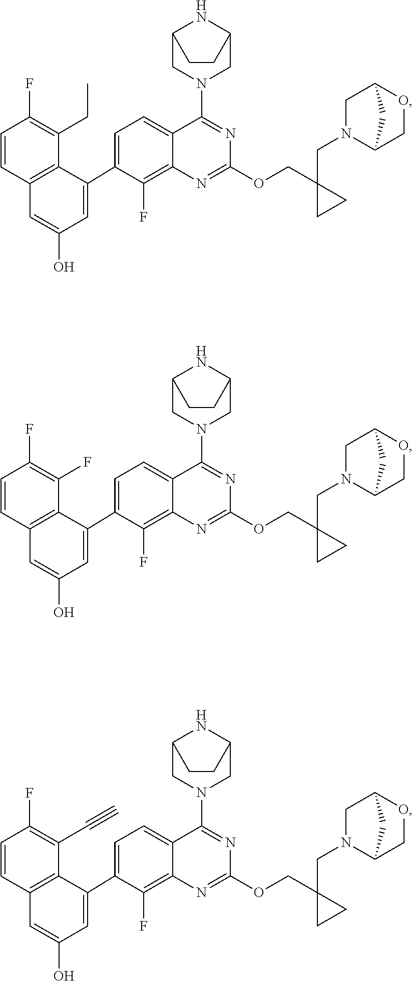 [SMILES: CCc1c(F)ccc2cc(O)cc(-c3ccc4c(N5CC6CCC(C5)N6)nc(OCC5(CN6C[C@@H]7C[C@H]6CO7)CC5)nc4c3F)c12 ; Oc1cc(-c2ccc3c(N4CC5CCC(C4)N5)nc(OCC4(CN5C[C@@H]6C[C@H]5CO6)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@@H]7C[C@H]6CO7)CC5)nc4c3F)c12]

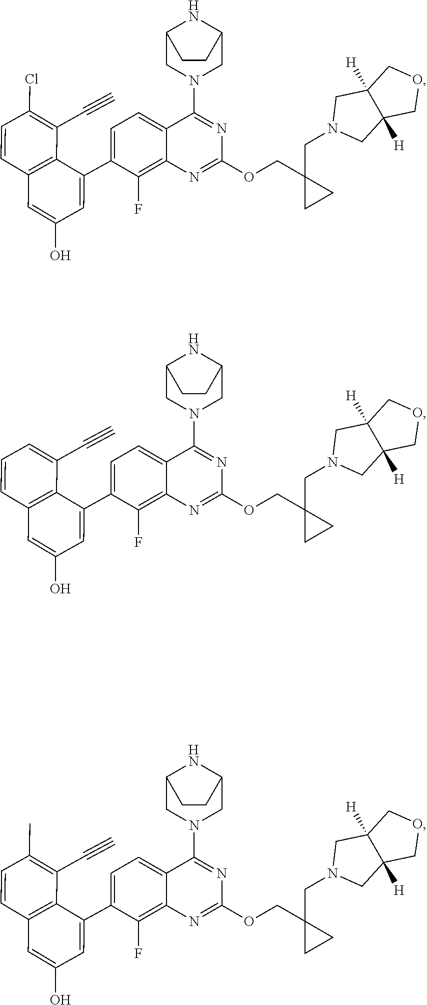 [SMILES: C#Cc1c(Cl)ccc2cc(O)cc(-c3ccc4c(N5CC6CCC(C5)N6)nc(OCC5(CN6C[C@H]7COC[C@@H]7C6)CC5)nc4c3F)c12 ; C#Cc1cccc2cc(O)cc(-c3ccc4c(N5CC6CCC(C5)N6)nc(OCC5(CN6C[C@H]7COC[C@@H]7C6)CC5)nc4c3F)c12 ; C#Cc1c(C)ccc2cc(O)cc(-c3ccc4c(N5CC6CCC(C5)N6)nc(OCC5(CN6C[C@H]7COC[C@@H]7C6)CC5)nc4c3F)c12]

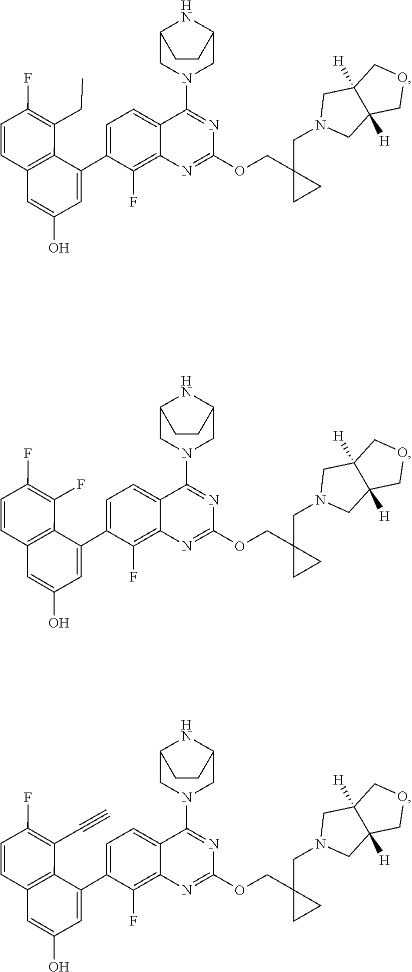 [SMILES: CCc1c(F)ccc2cc(O)cc(-c3ccc4c(N5CC6CCC(C5)N6)nc(OCC5(CN6C[C@H]7COC[C@@H]7C6)CC5)nc4c3F)c12 ; Oc1cc(-c2ccc3c(N4CC5CCC(C4)N5)nc(OCC4(CN5C[C@H]6COC[C@@H]6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7COC[C@@H]7C6)CC5)nc4c3F)c12]

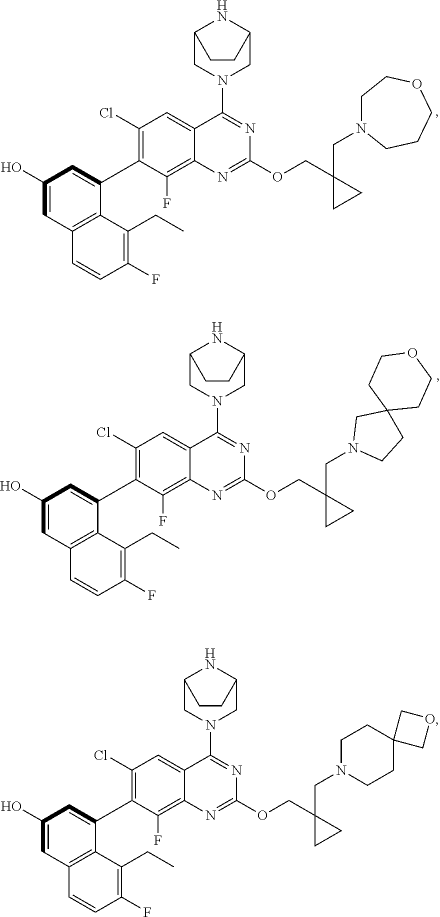

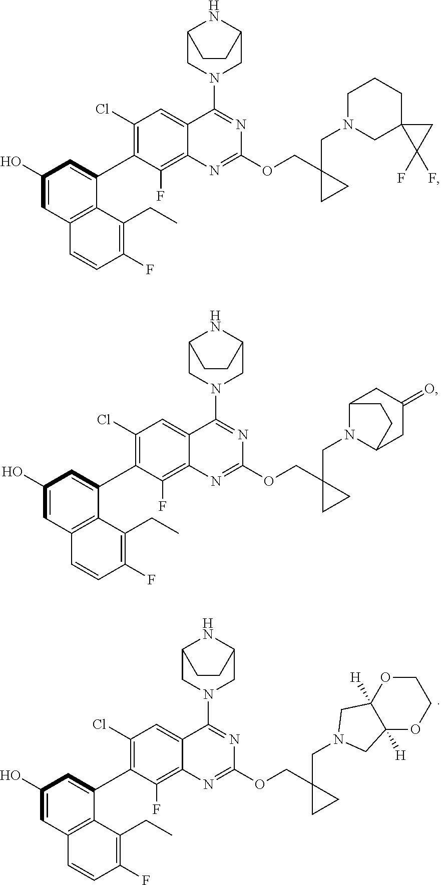

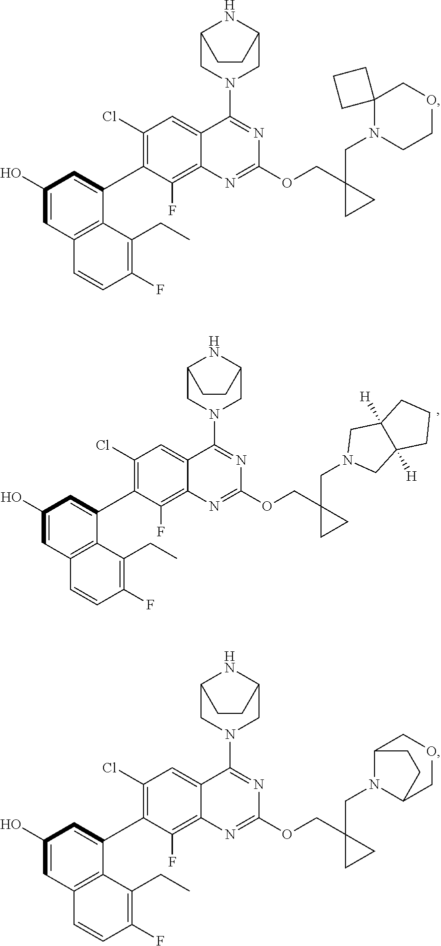

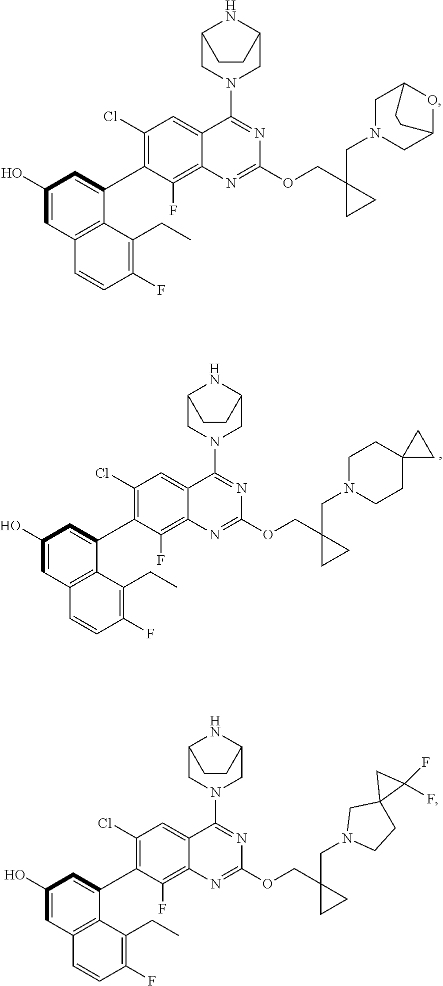

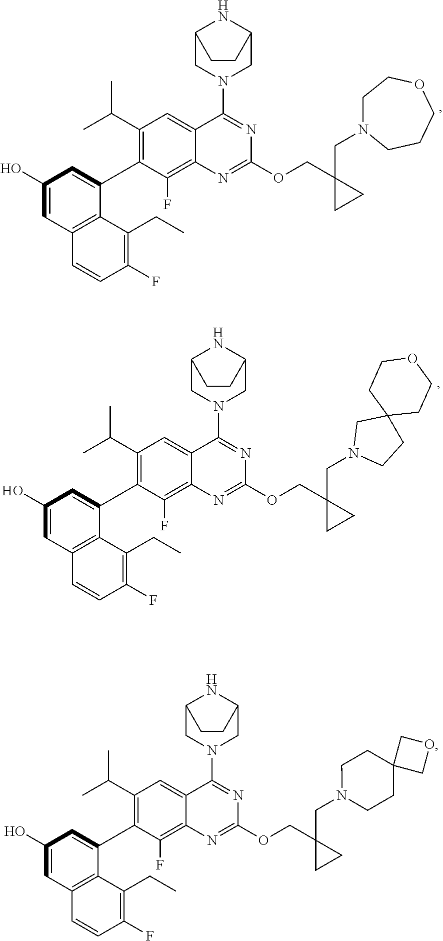

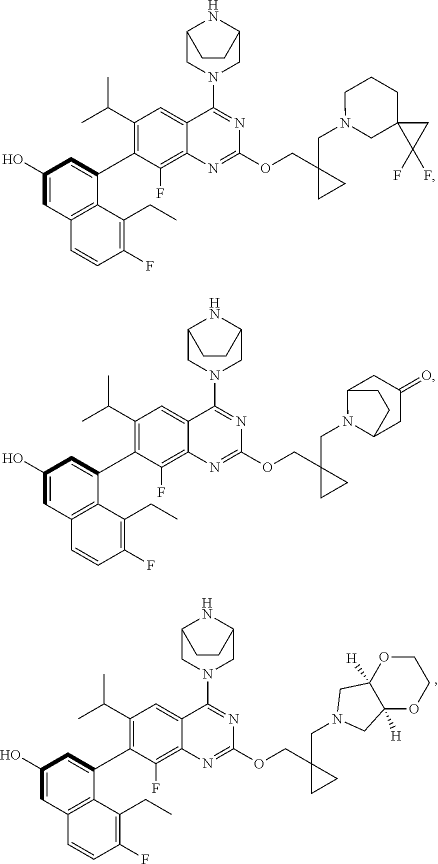

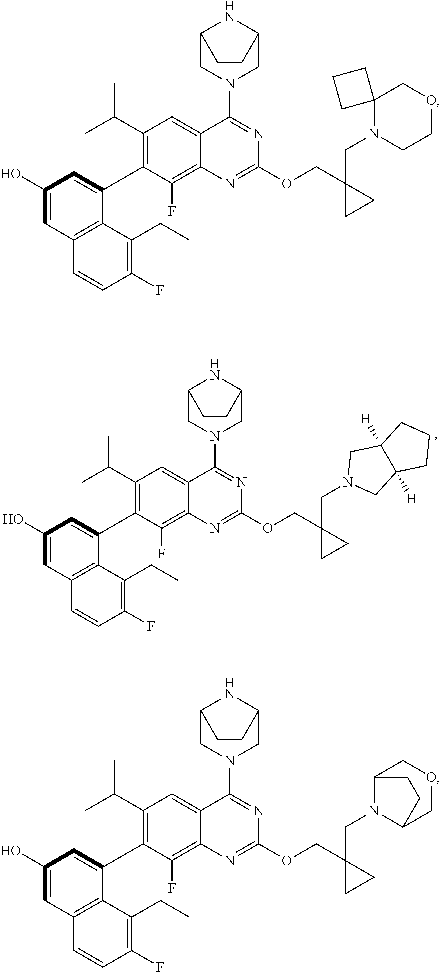

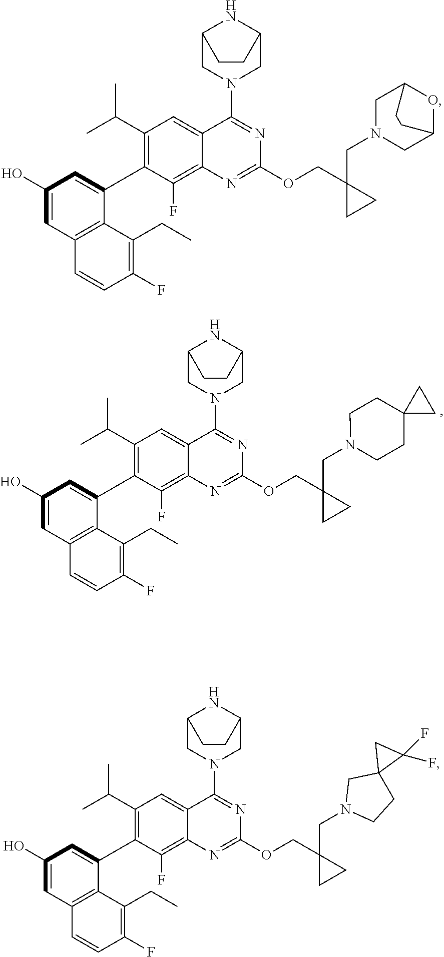

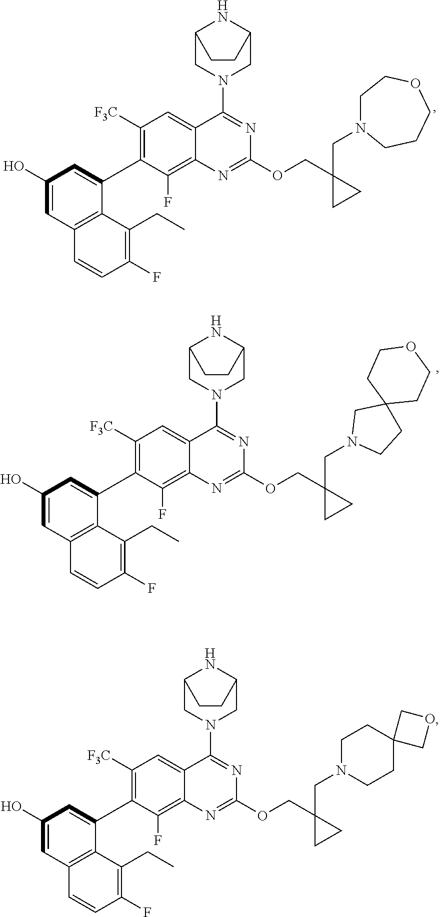

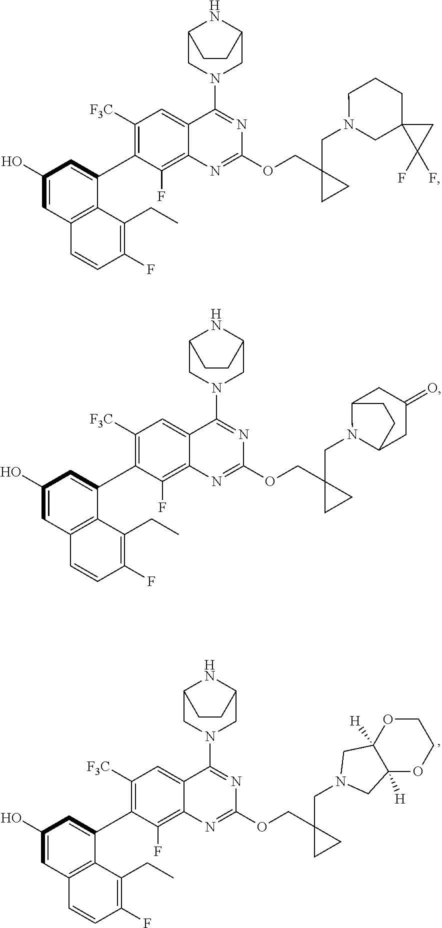

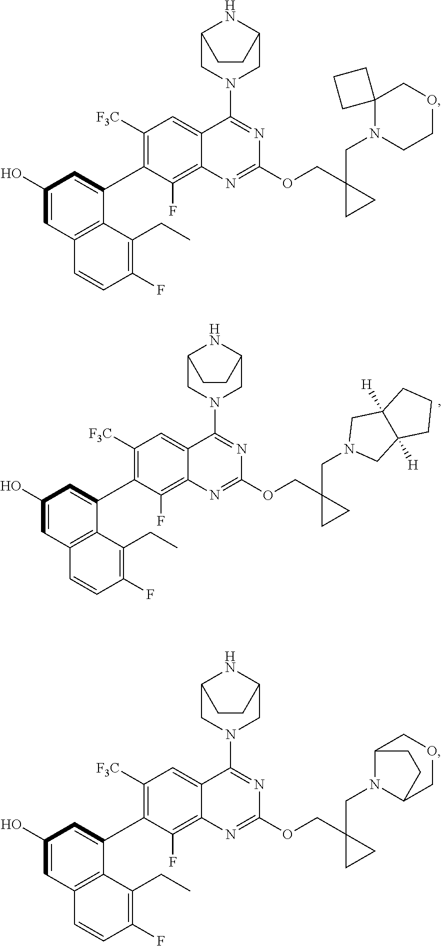

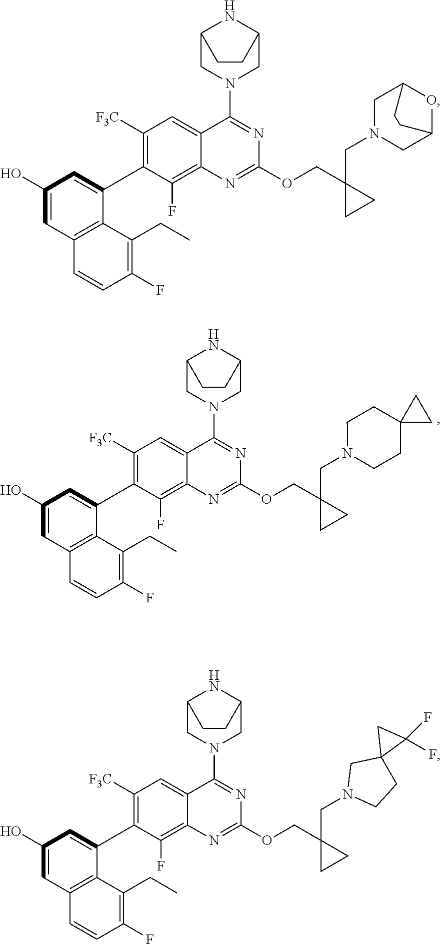

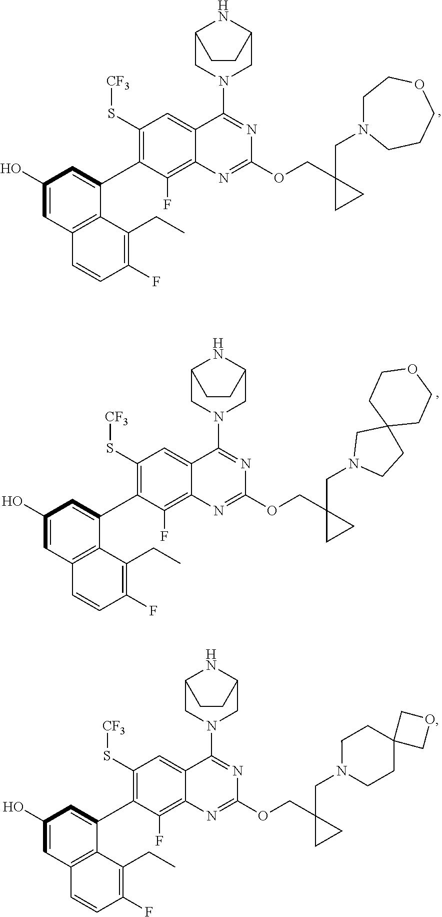

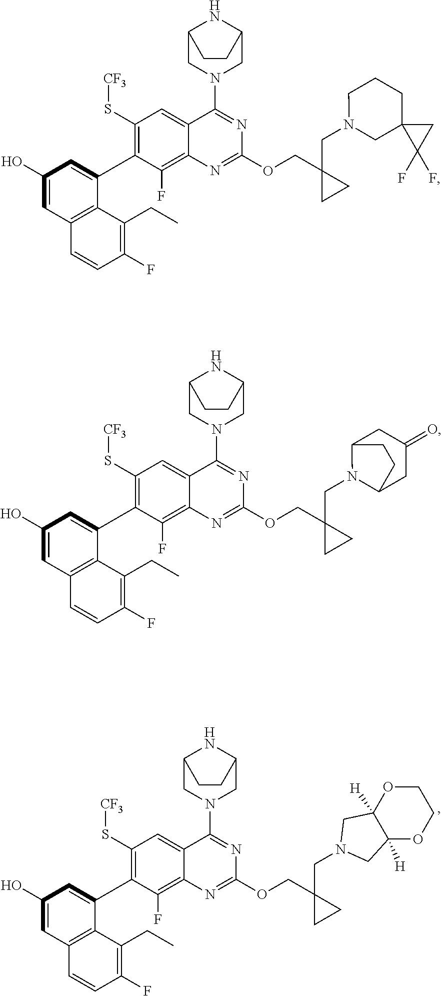

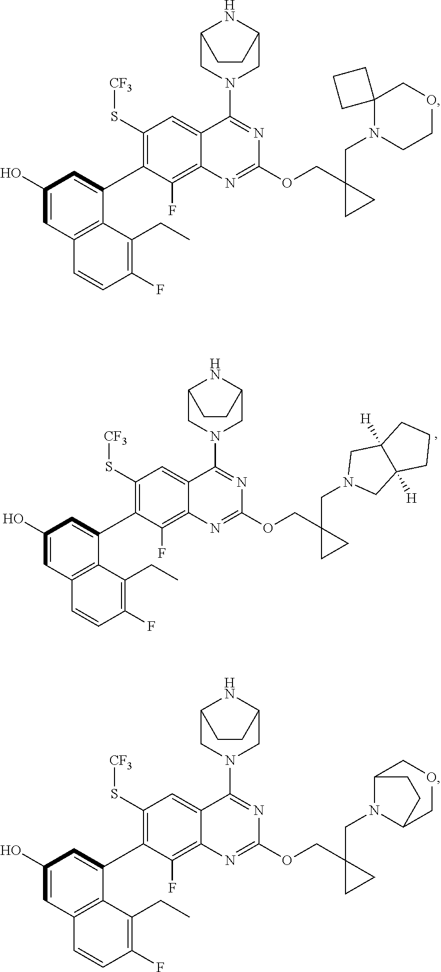

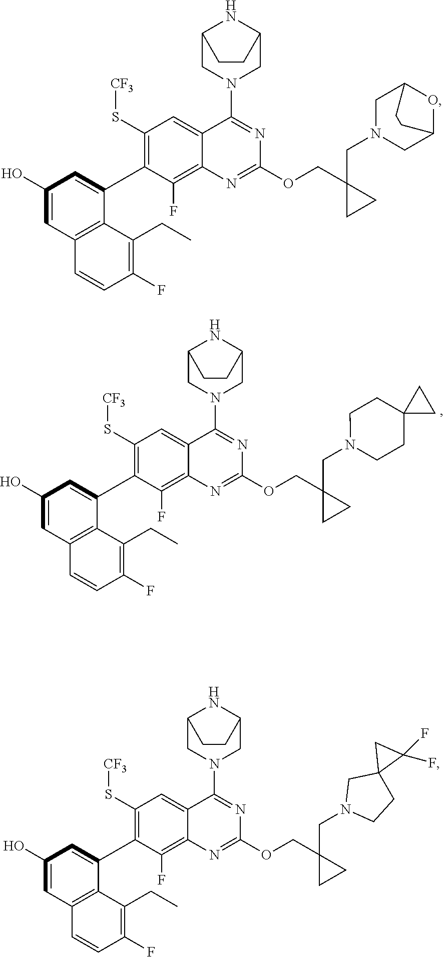

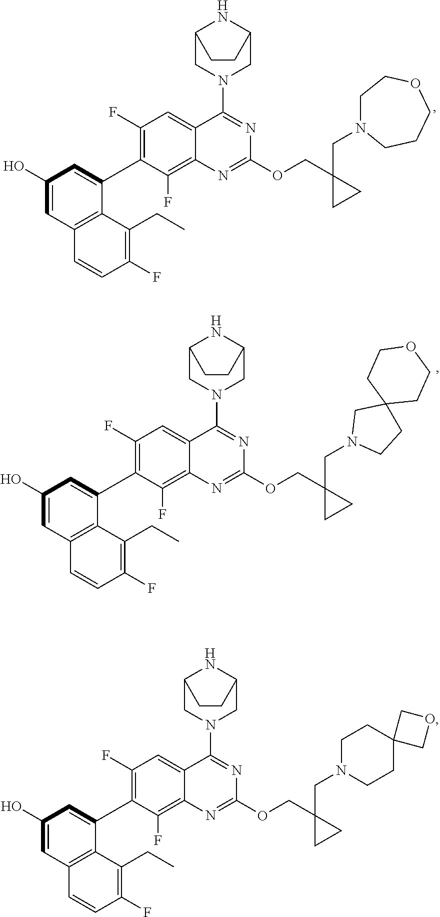

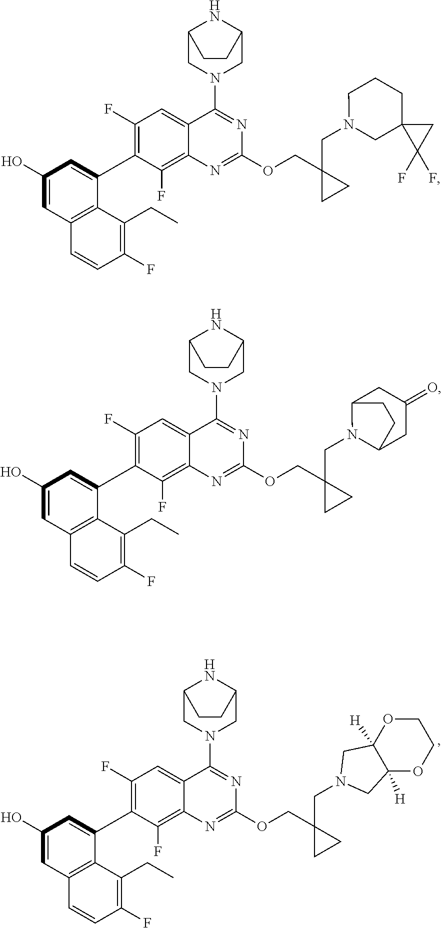

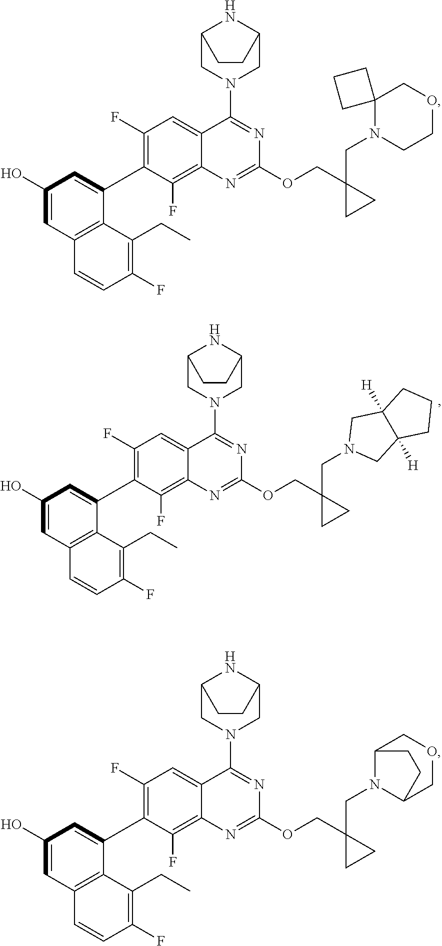

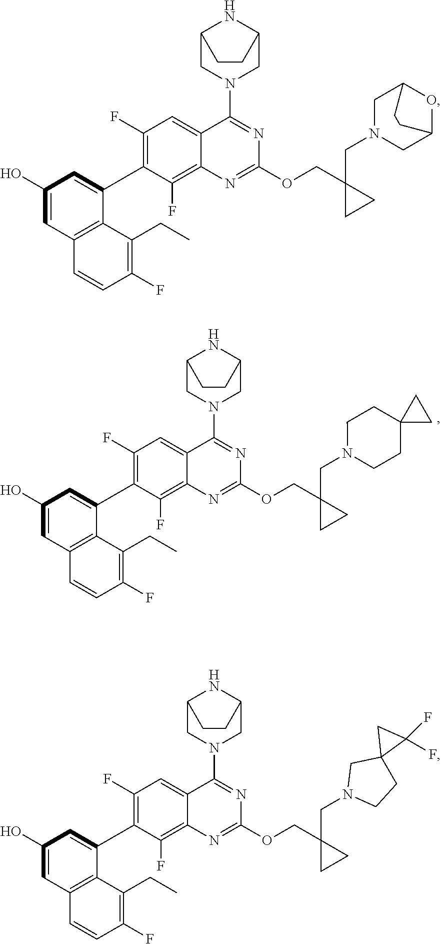

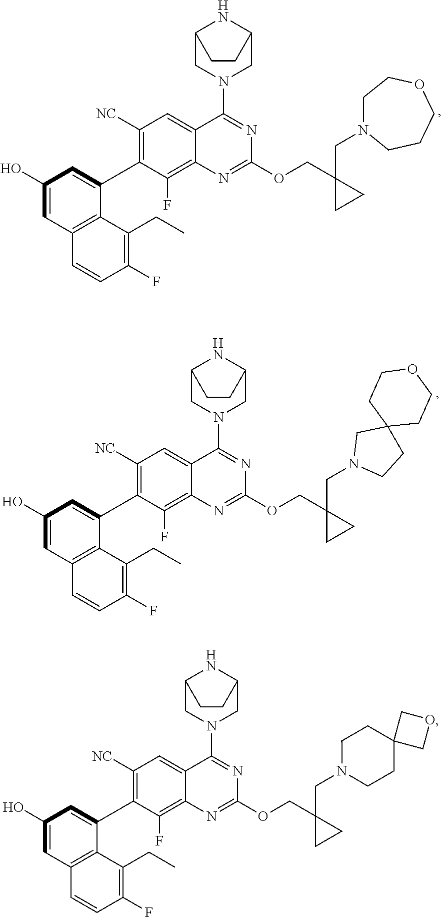

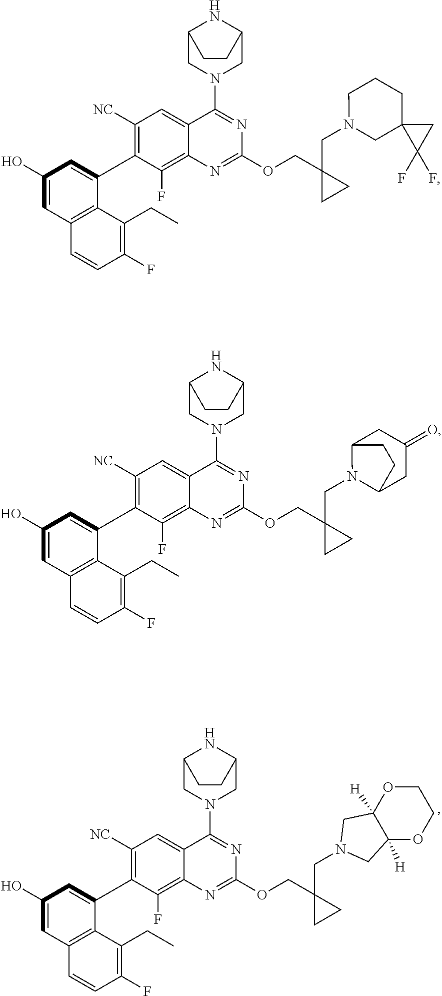

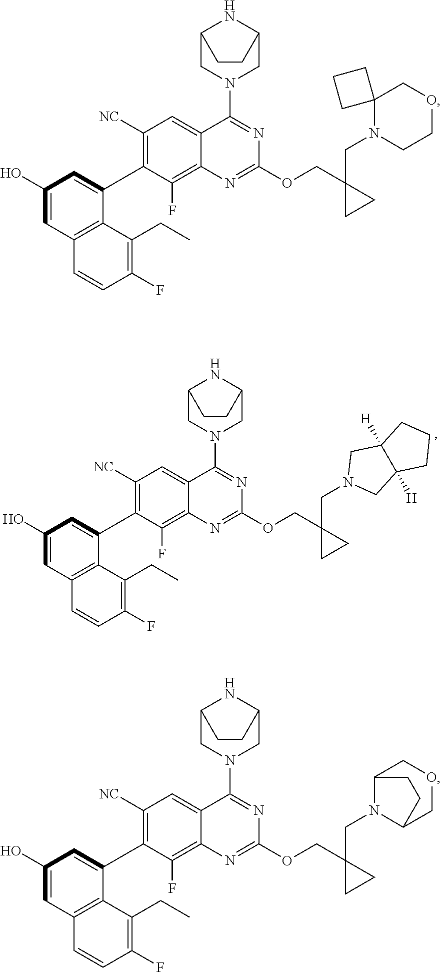

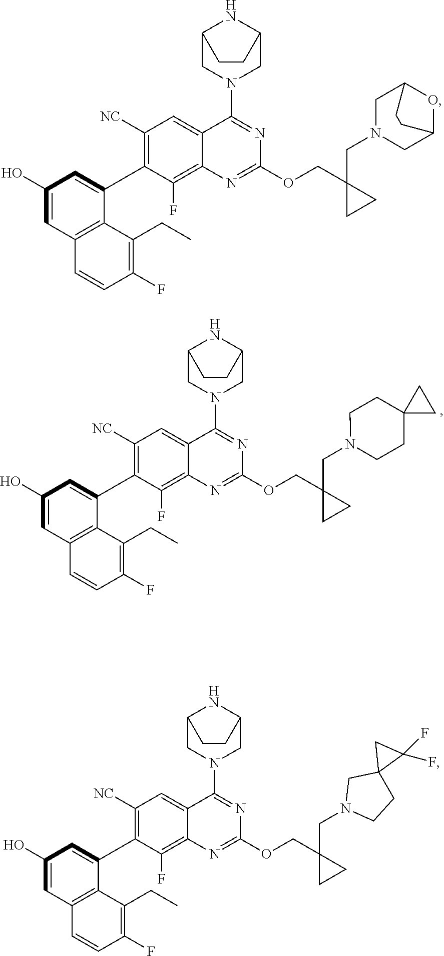

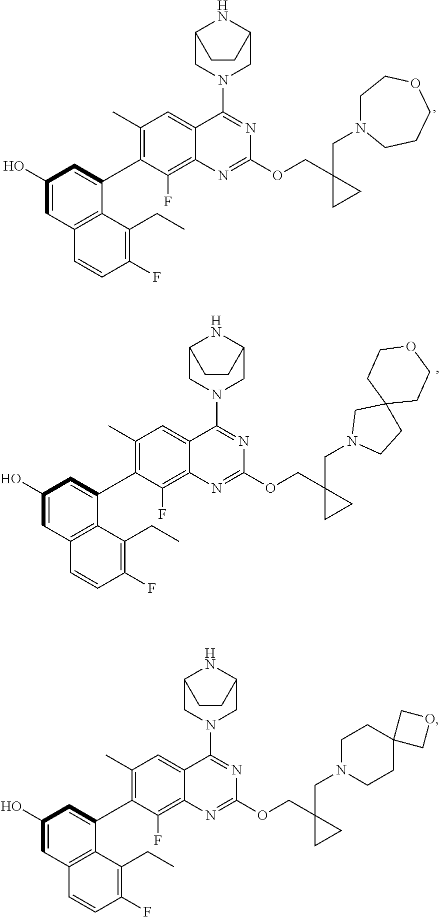

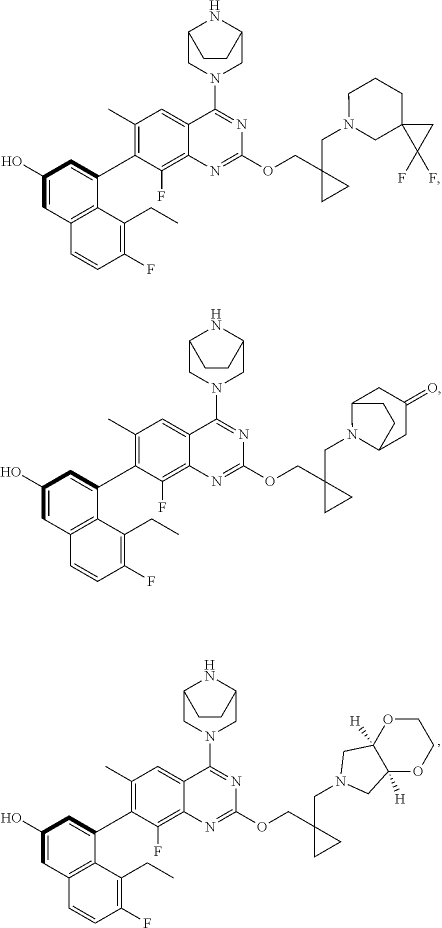

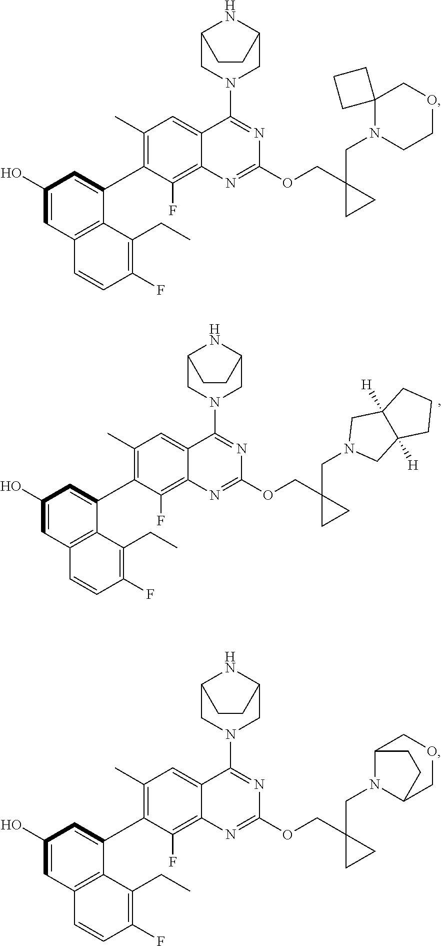

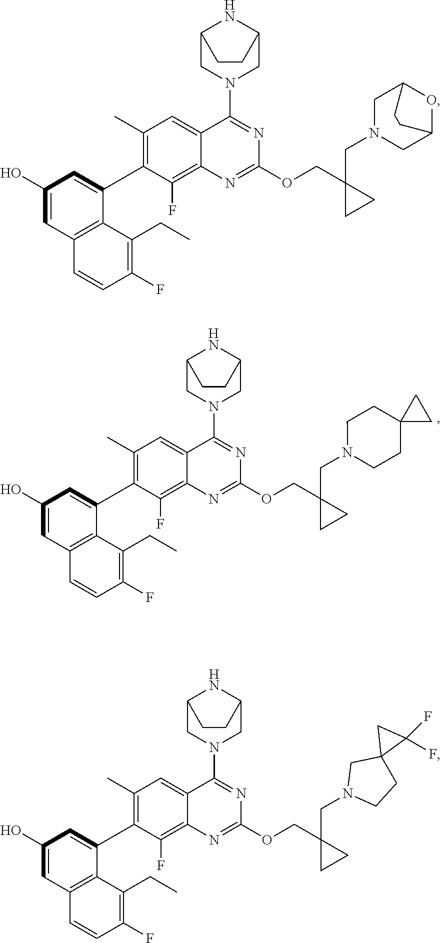

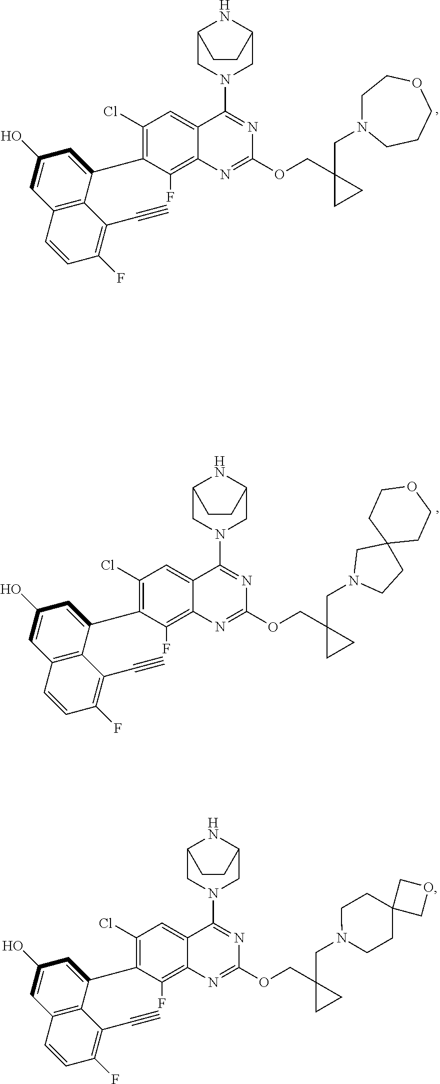

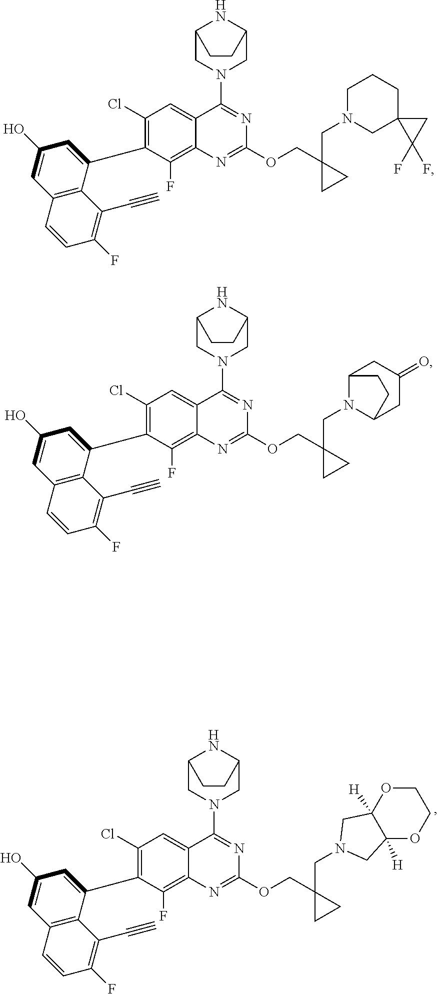

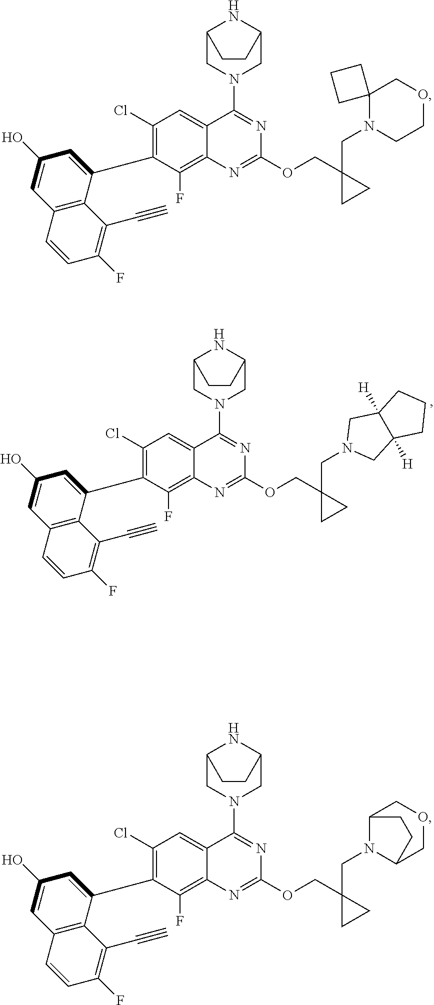

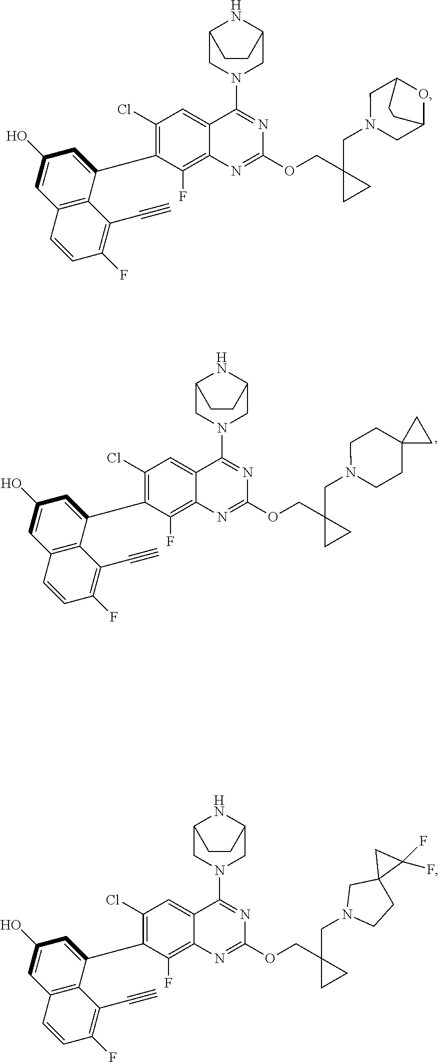

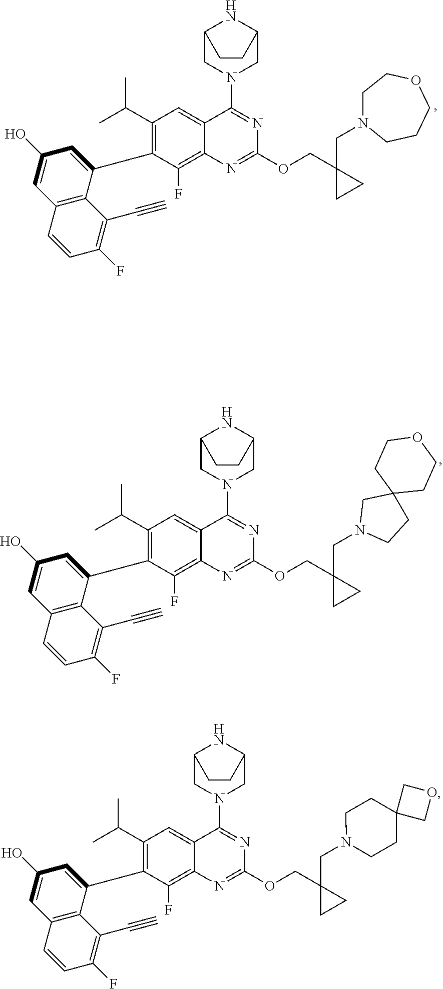

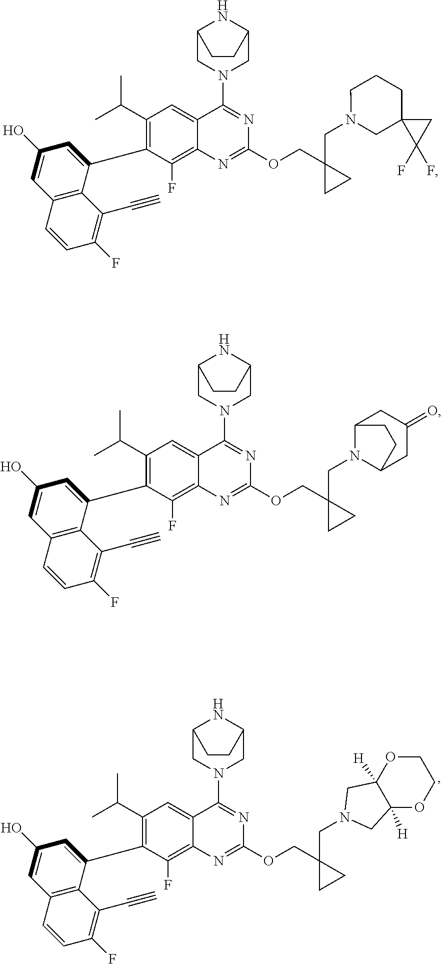

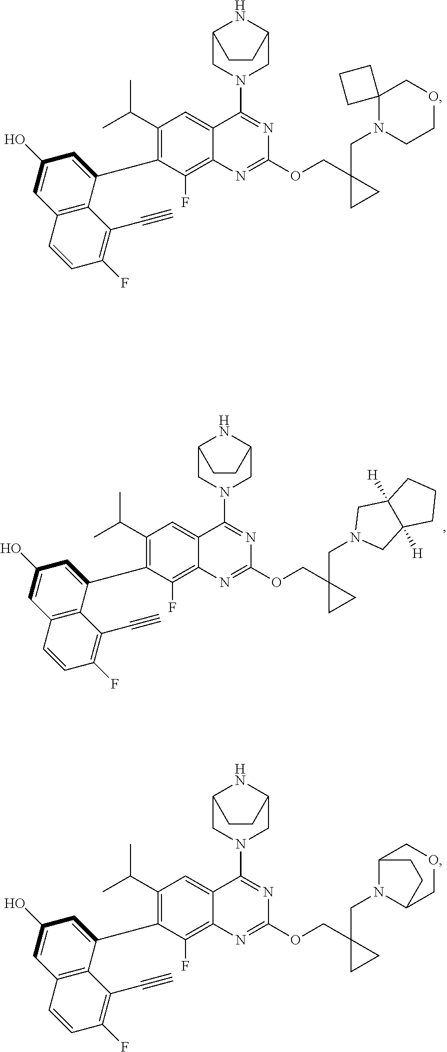

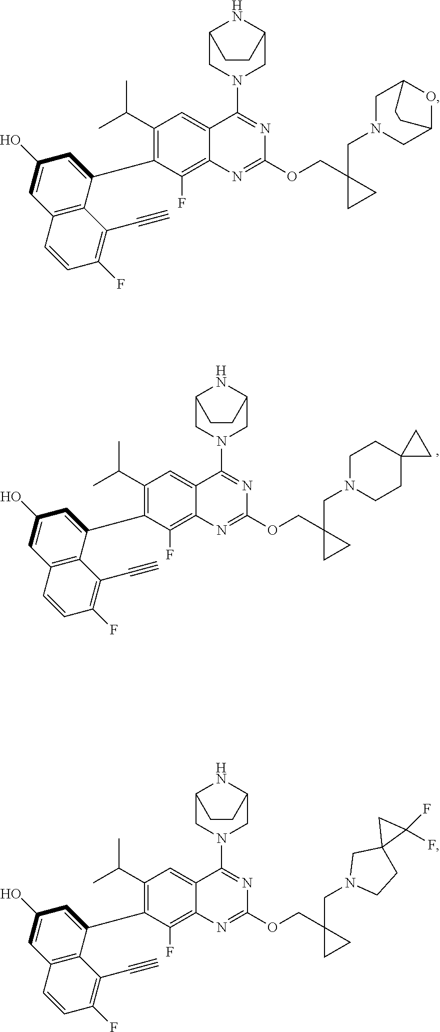

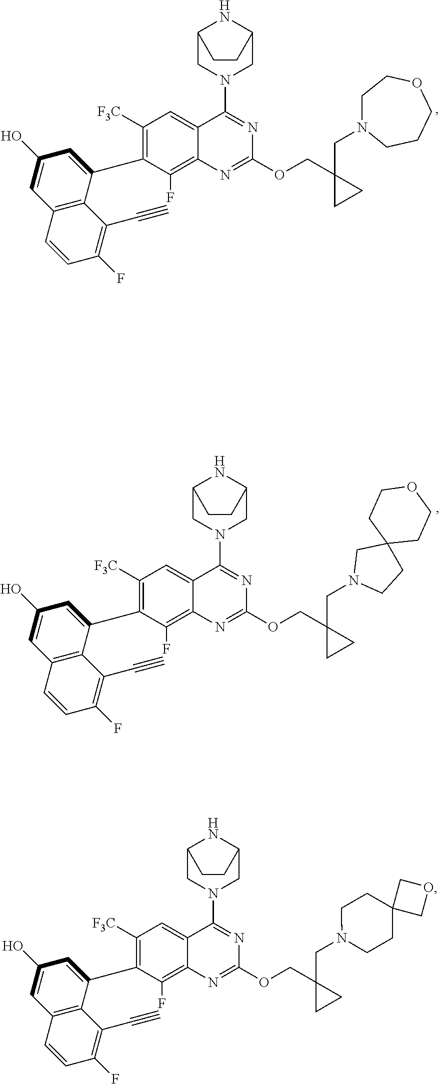

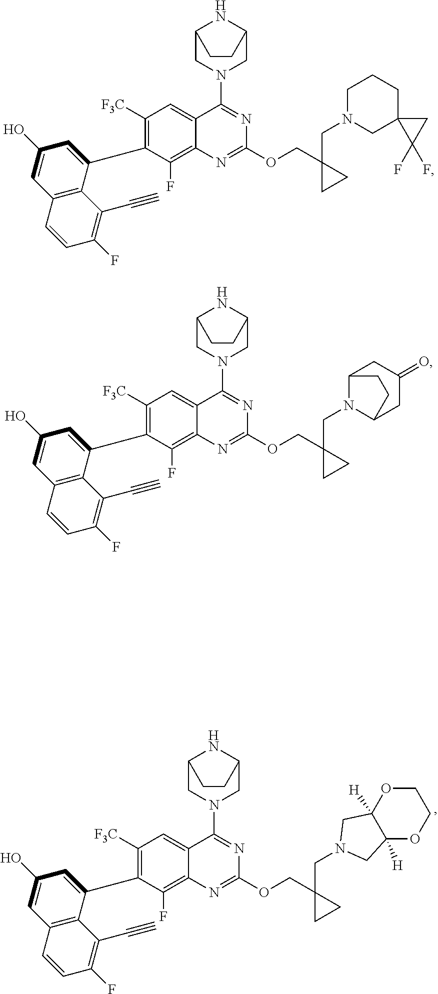

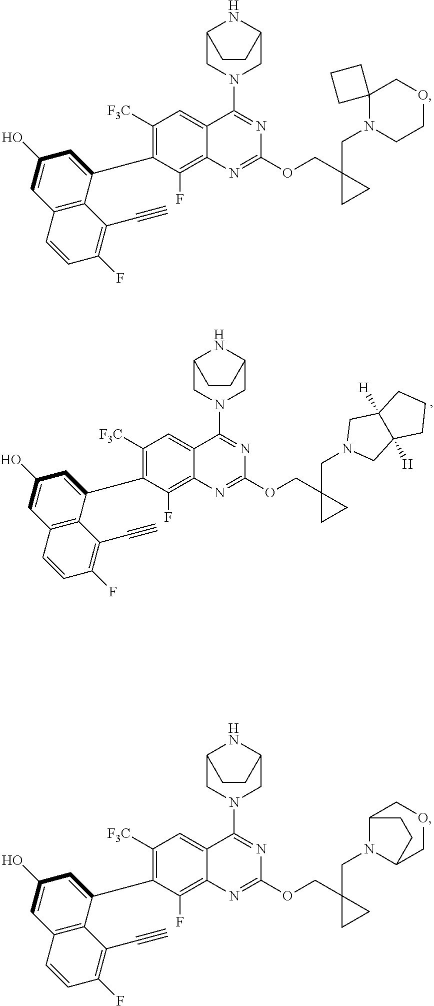

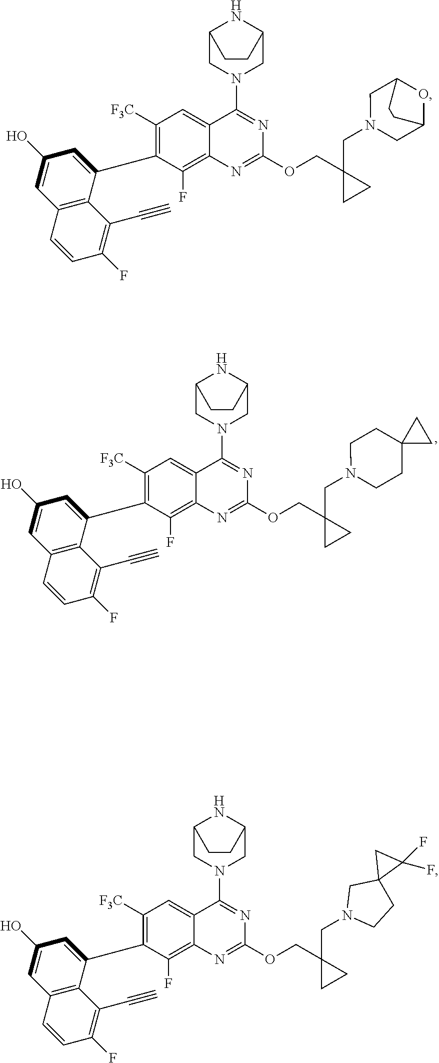

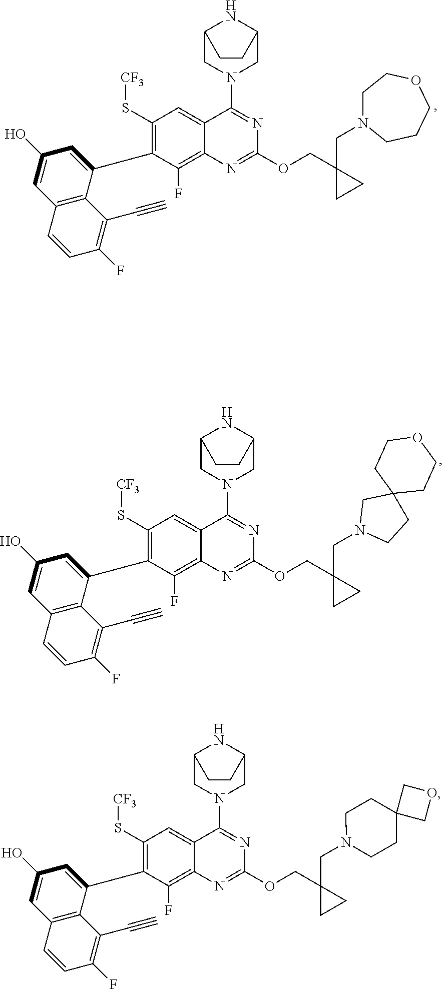

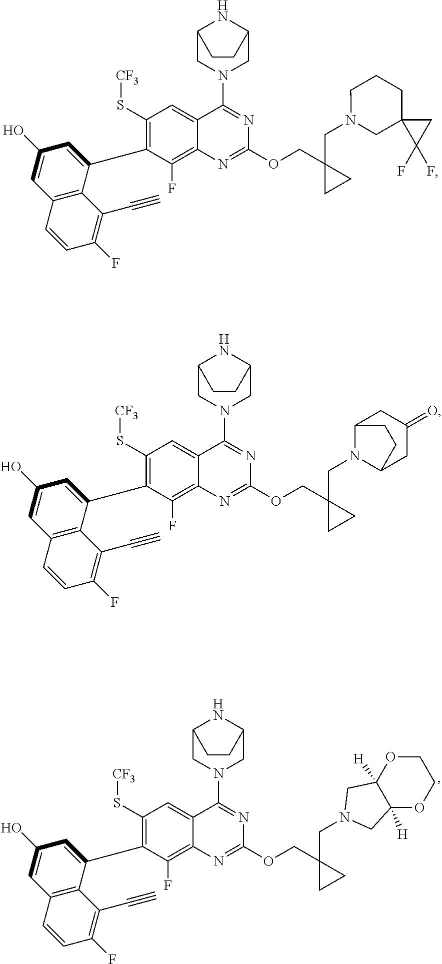

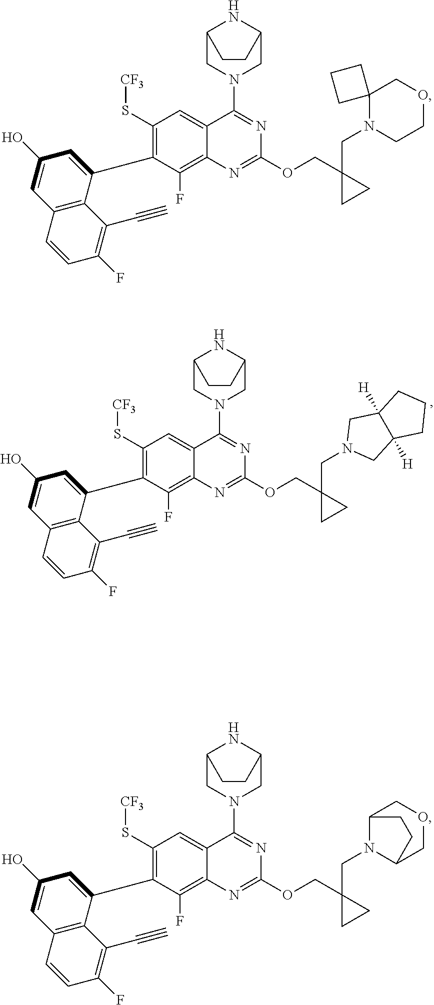

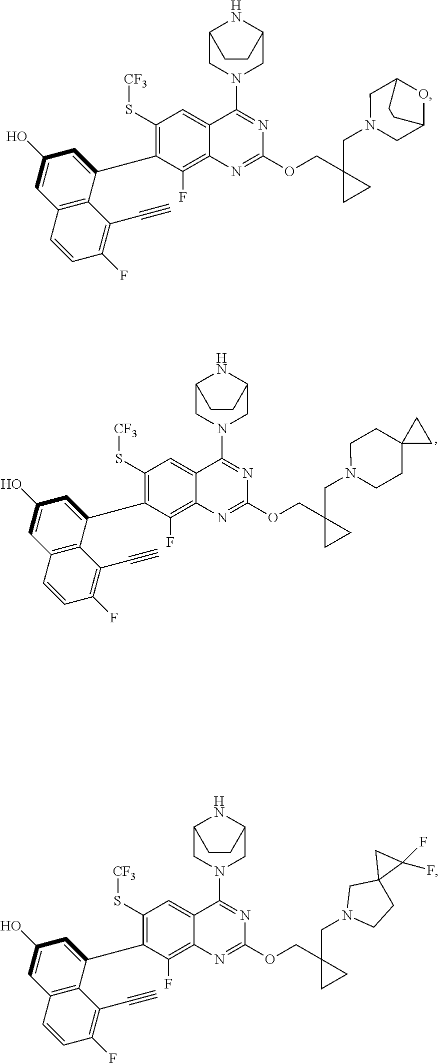

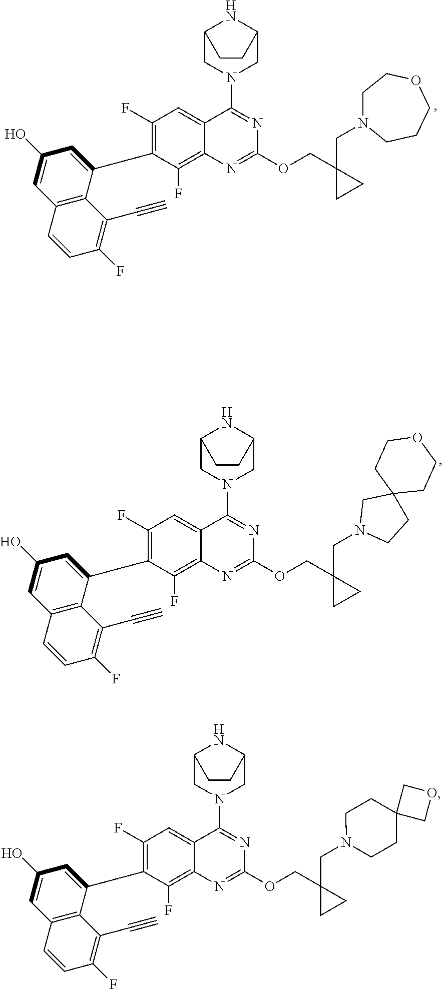

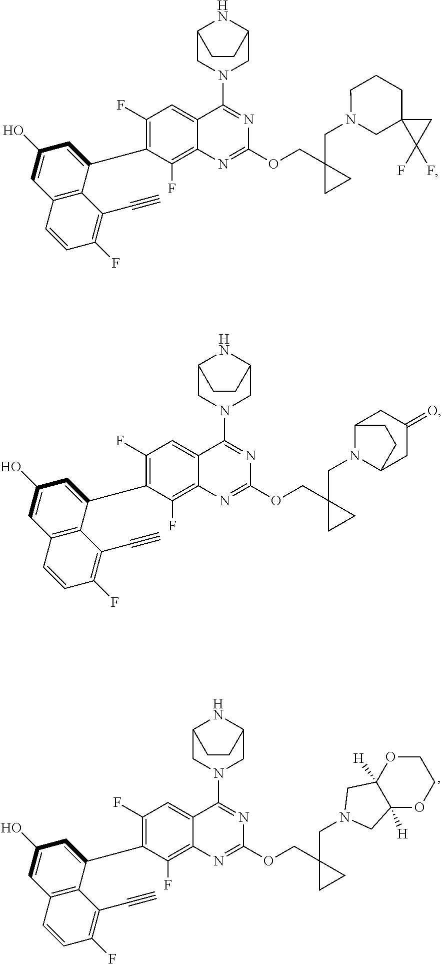

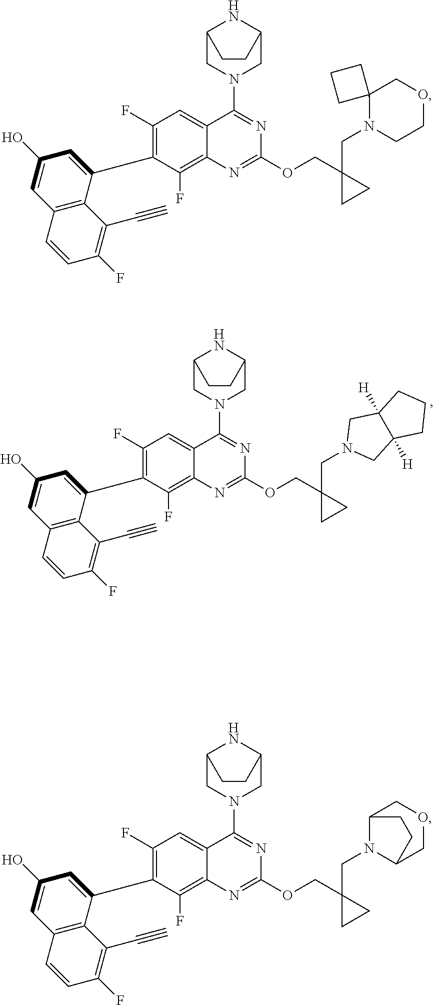

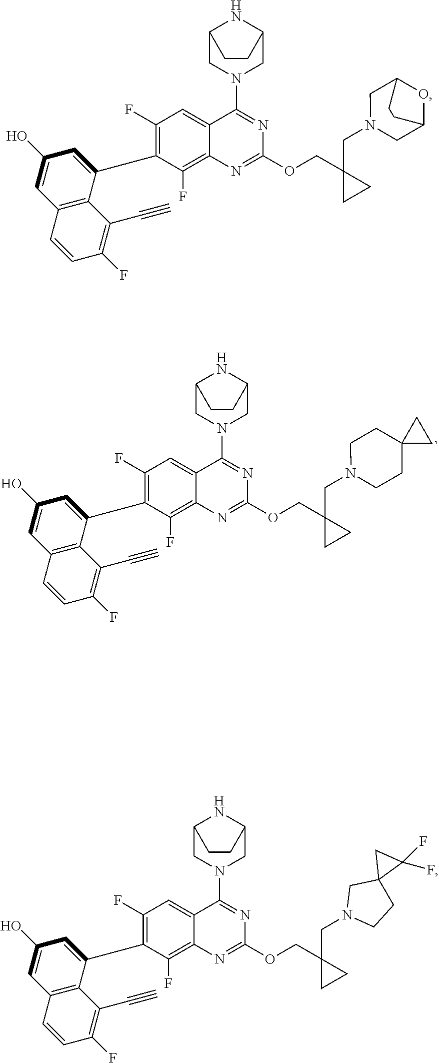

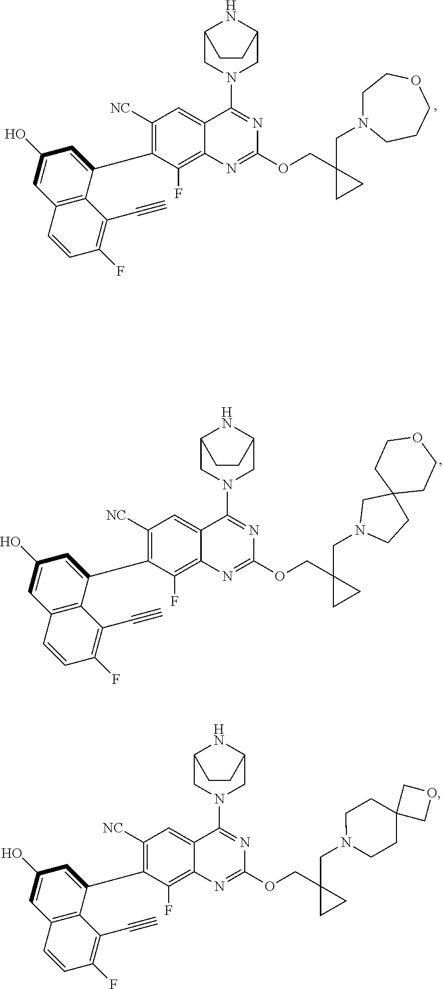

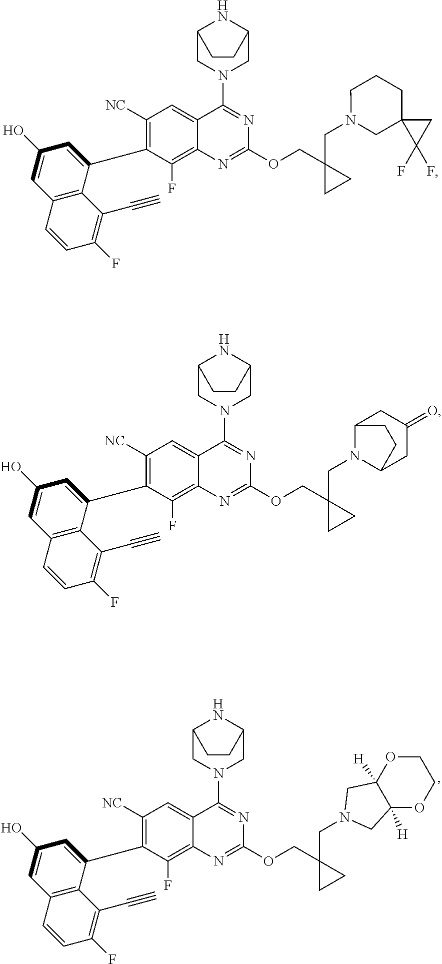

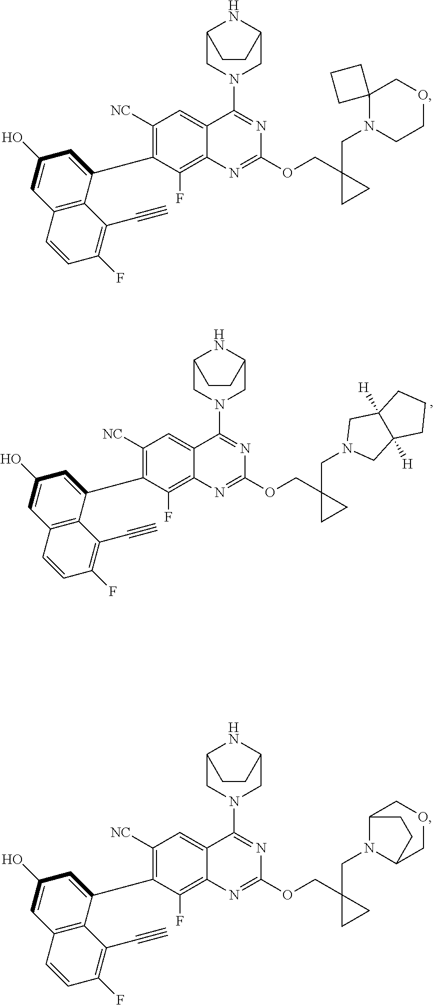

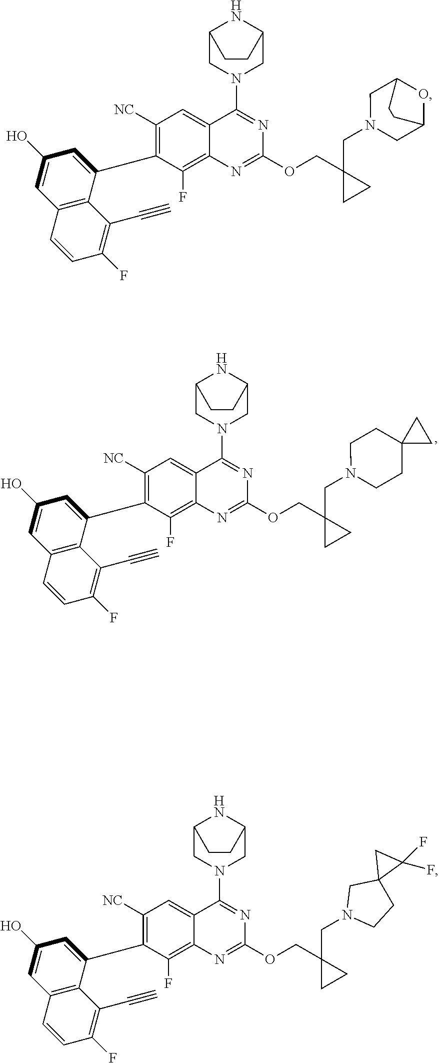

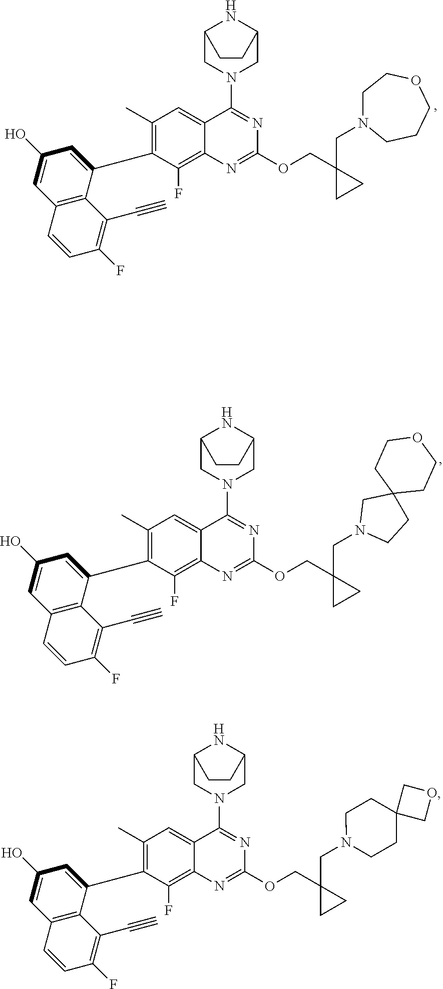

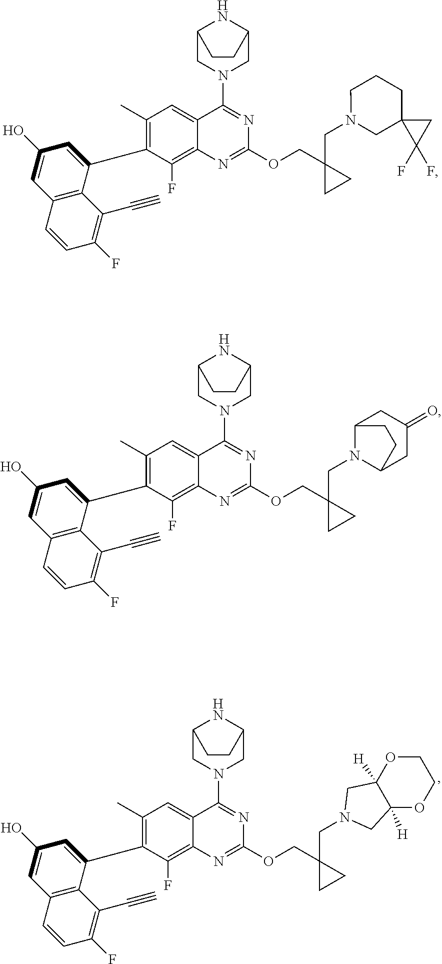

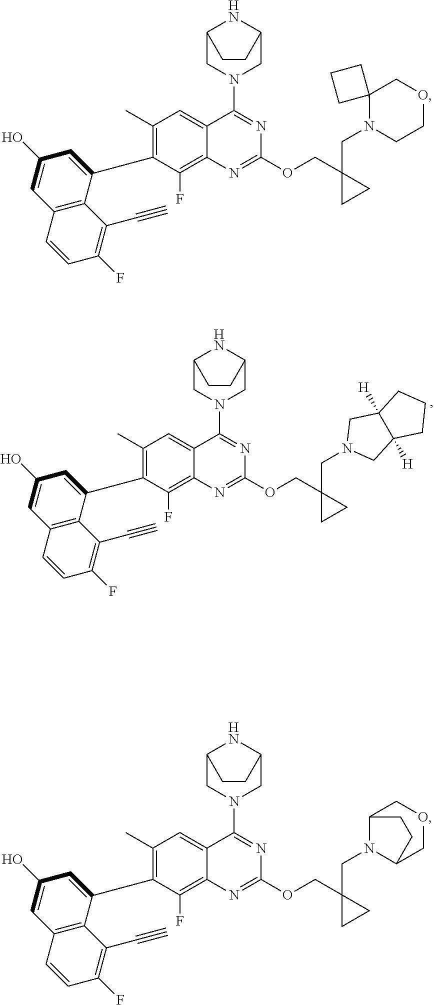

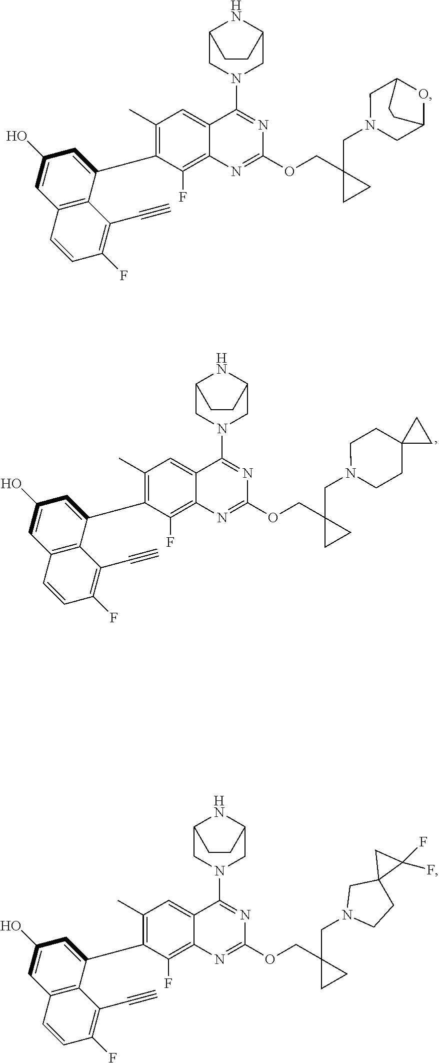

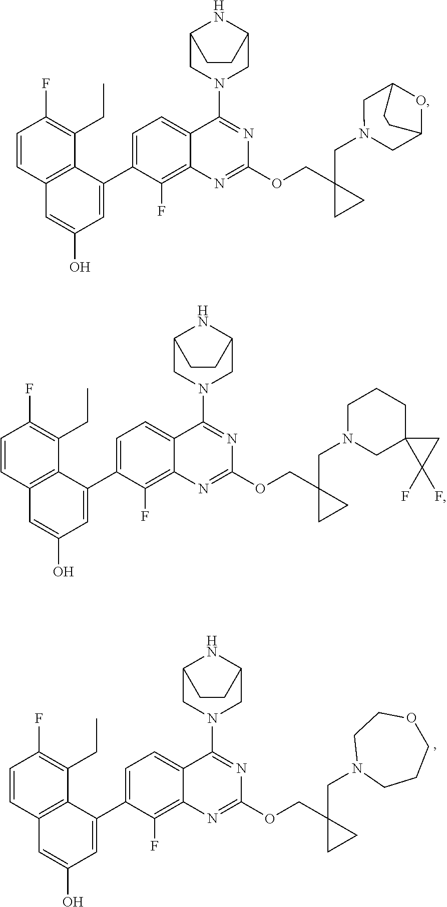 [SMILES: CCc1c(F)ccc2cc(O)cc(-c3ccc4c(N5CC6CCC(C5)N6)nc(OCC5(CN6CC7CCC(C6)O7)CC5)nc4c3F)c12 ; CCc1c(F)ccc2cc(O)cc(-c3ccc4c(N5CC6CCC(C5)N6)nc(OCC5(CN6CCCC7(C6)CC7(F)F)CC5)nc4c3F)c12 ; CCc1c(F)ccc2cc(O)cc(-c3ccc4c(N5CC6CCC(C5)N6)nc(OCC5(CN6CCCOCC6)CC5)nc4c3F)c12]

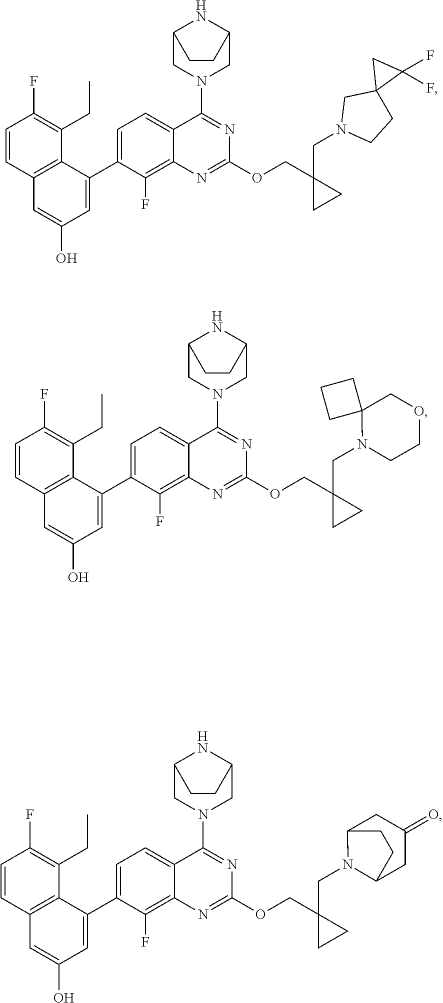 [SMILES: CCc1c(F)ccc2cc(O)cc(-c3ccc4c(N5CC6CCC(C5)N6)nc(OCC5(CN6CCC7(C6)CC7(F)F)CC5)nc4c3F)c12 ; CCc1c(F)ccc2cc(O)cc(-c3ccc4c(N5CC6CCC(C5)N6)nc(OCC5(CN6CCOCC67CCC7)CC5)nc4c3F)c12 ; CCc1c(F)ccc2cc(O)cc(-c3ccc4c(N5CC6CCC(C5)N6)nc(OCC5(CN6C7CCC6CC(=O)C7)CC5)nc4c3F)c12]

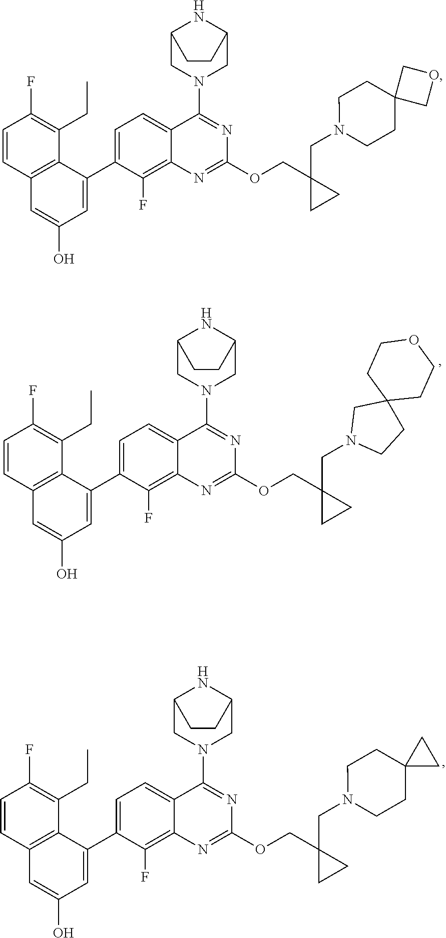 [SMILES: CCc1c(F)ccc2cc(O)cc(-c3ccc4c(N5CC6CCC(C5)N6)nc(OCC5(CN6CCC7(CC6)COC7)CC5)nc4c3F)c12 ; CCc1c(F)ccc2cc(O)cc(-c3ccc4c(N5CC6CCC(C5)N6)nc(OCC5(CN6CCC7(CCOCC7)C6)CC5)nc4c3F)c12 ; CCc1c(F)ccc2cc(O)cc(-c3ccc4c(N5CC6CCC(C5)N6)nc(OCC5(CN6CCC7(CC6)CC7)CC5)nc4c3F)c12]

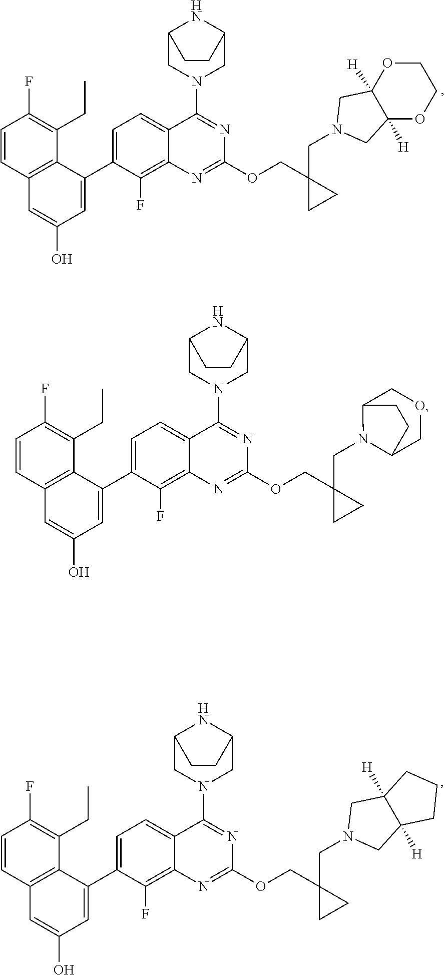 [SMILES: CCc1c(F)ccc2cc(O)cc(-c3ccc4c(N5CC6CCC(C5)N6)nc(OCC5(CN6C[C@@H]7OCCO[C@@H]7C6)CC5)nc4c3F)c12 ; CCc1c(F)ccc2cc(O)cc(-c3ccc4c(N5CC6CCC(C5)N6)nc(OCC5(CN6C7CCC6COC7)CC5)nc4c3F)c12 ; CCc1c(F)ccc2cc(O)cc(-c3ccc4c(N5CC6CCC(C5)N6)nc(OCC5(CN6C[C@H]7CCC[C@H]7C6)CC5)nc4c3F)c12]

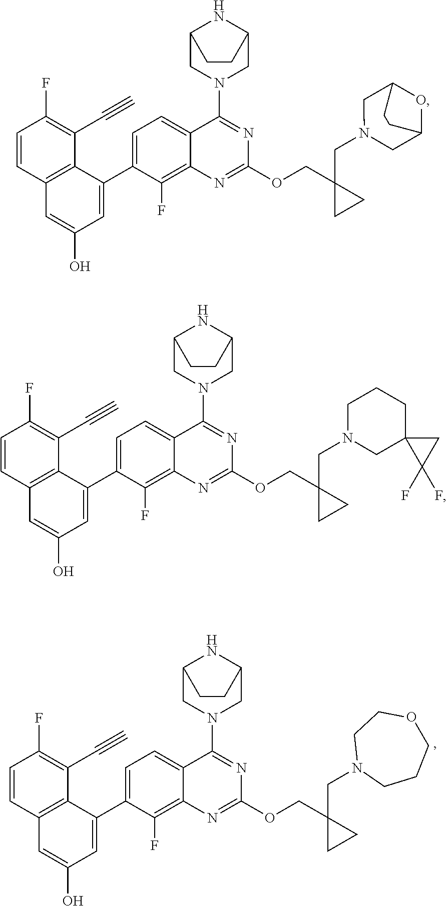 [SMILES: C#Cc1c(F)ccc2cc(O)cc(-c3ccc4c(N5CC6CCC(C5)N6)nc(OCC5(CN6CC7CCC(C6)O7)CC5)nc4c3F)c12 ; C#Cc1c(F)ccc2cc(O)cc(-c3ccc4c(N5CC6CCC(C5)N6)nc(OCC5(CN6CCCC7(C6)CC7(F)F)CC5)nc4c3F)c12 ; C#Cc1c(F)ccc2cc(O)cc(-c3ccc4c(N5CC6CCC(C5)N6)nc(OCC5(CN6CCCOCC6)CC5)nc4c3F)c12]

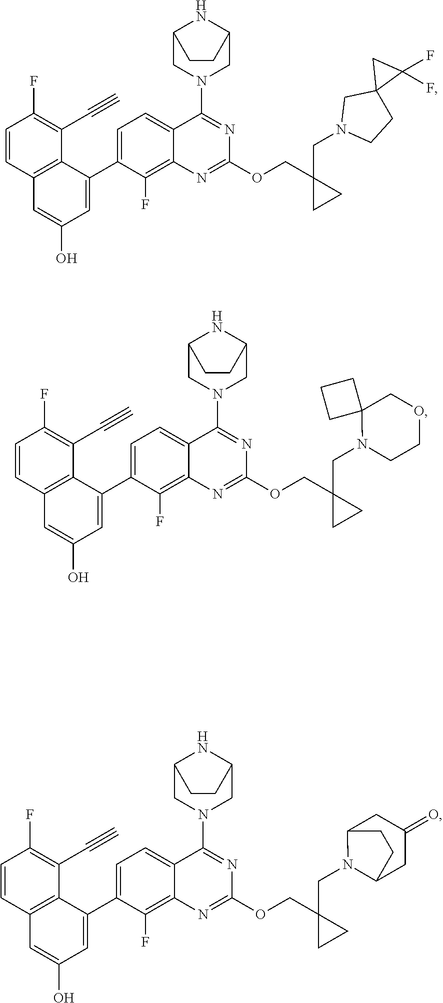 [SMILES: C#Cc1c(F)ccc2cc(O)cc(-c3ccc4c(N5CC6CCC(C5)N6)nc(OCC5(CN6CCC7(C6)CC7(F)F)CC5)nc4c3F)c12 ; C#Cc1c(F)ccc2cc(O)cc(-c3ccc4c(N5CC6CCC(C5)N6)nc(OCC5(CN6CCOCC67CCC7)CC5)nc4c3F)c12 ; C#Cc1c(F)ccc2cc(O)cc(-c3ccc4c(N5CC6CCC(C5)N6)nc(OCC5(CN6C7CCC6CC(=O)C7)CC5)nc4c3F)c12]

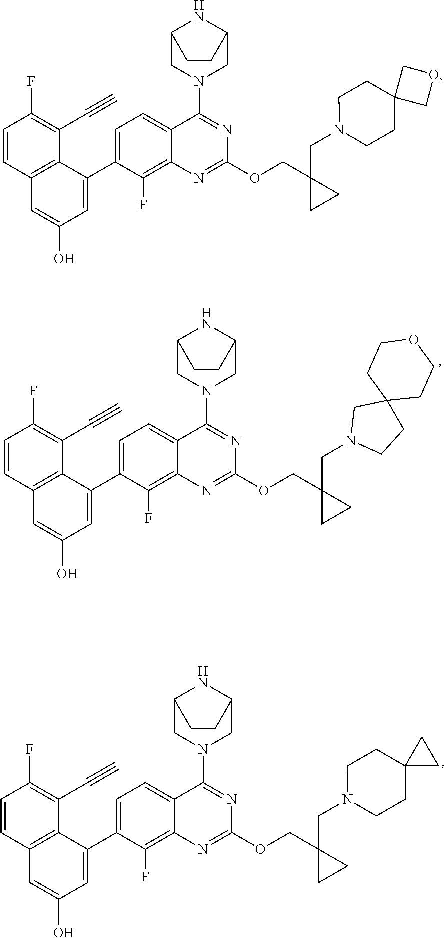 [SMILES: C#Cc1c(F)ccc2cc(O)cc(-c3ccc4c(N5CC6CCC(C5)N6)nc(OCC5(CN6CCC7(CC6)COC7)CC5)nc4c3F)c12 ; C#Cc1c(F)ccc2cc(O)cc(-c3ccc4c(N5CC6CCC(C5)N6)nc(OCC5(CN6CCC7(CCOCC7)C6)CC5)nc4c3F)c12 ; C#Cc1c(F)ccc2cc(O)cc(-c3ccc4c(N5CC6CCC(C5)N6)nc(OCC5(CN6CCC7(CC6)CC7)CC5)nc4c3F)c12]

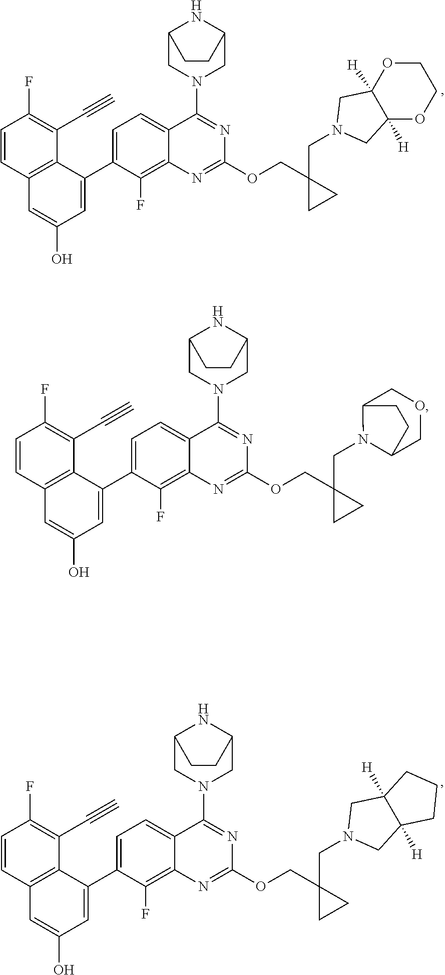 [SMILES: C#Cc1c(F)ccc2cc(O)cc(-c3ccc4c(N5CC6CCC(C5)N6)nc(OCC5(CN6C[C@@H]7OCCO[C@@H]7C6)CC5)nc4c3F)c12 ; C#Cc1c(F)ccc2cc(O)cc(-c3ccc4c(N5CC6CCC(C5)N6)nc(OCC5(CN6C7CCC6COC7)CC5)nc4c3F)c12 ; C#Cc1c(F)ccc2cc(O)cc(-c3ccc4c(N5CC6CCC(C5)N6)nc(OCC5(CN6C[C@H]7CCC[C@H]7C6)CC5)nc4c3F)c12]

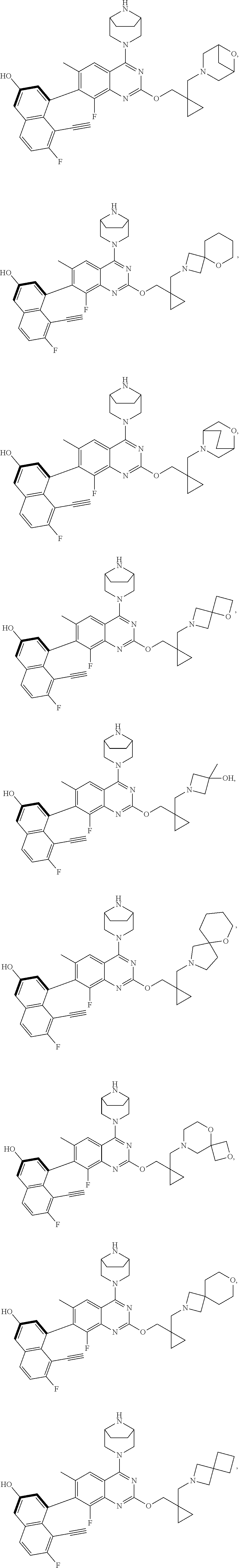

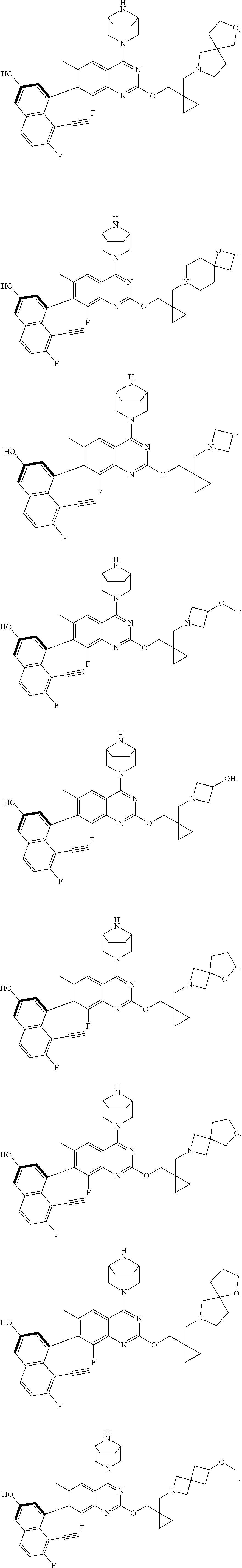

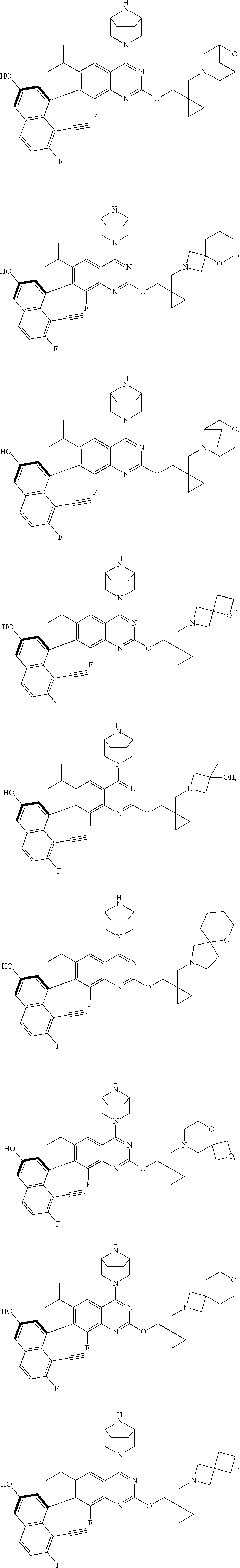

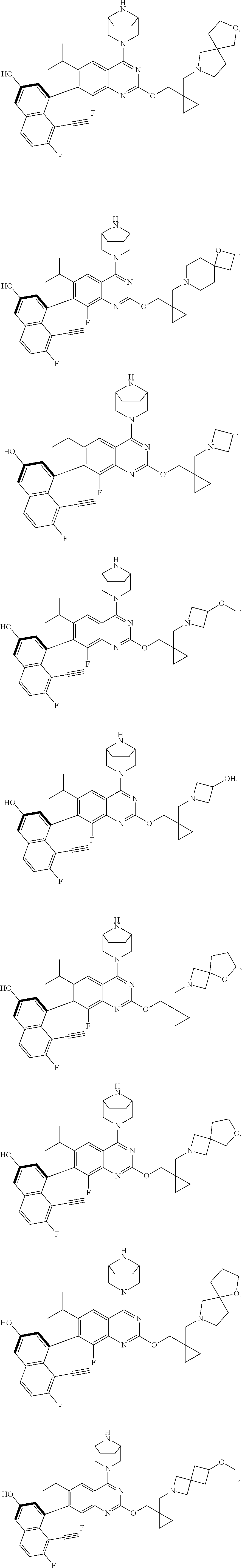

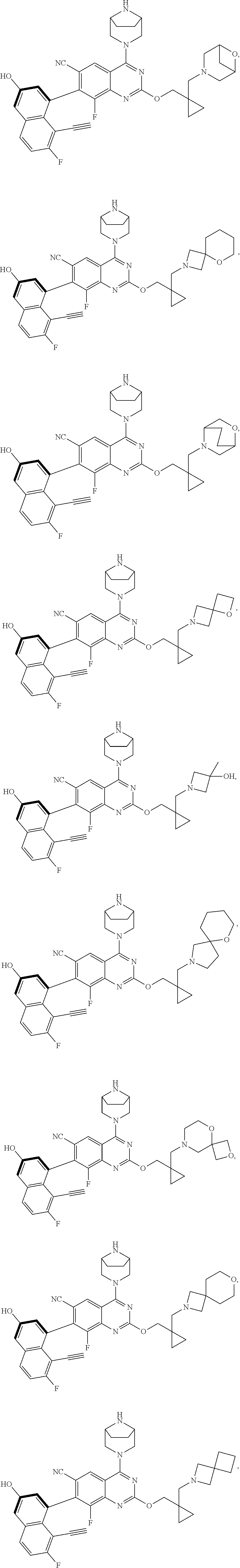

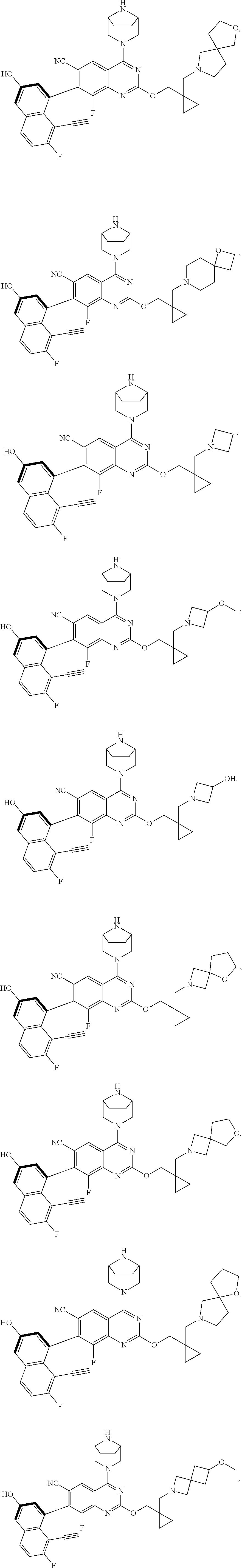

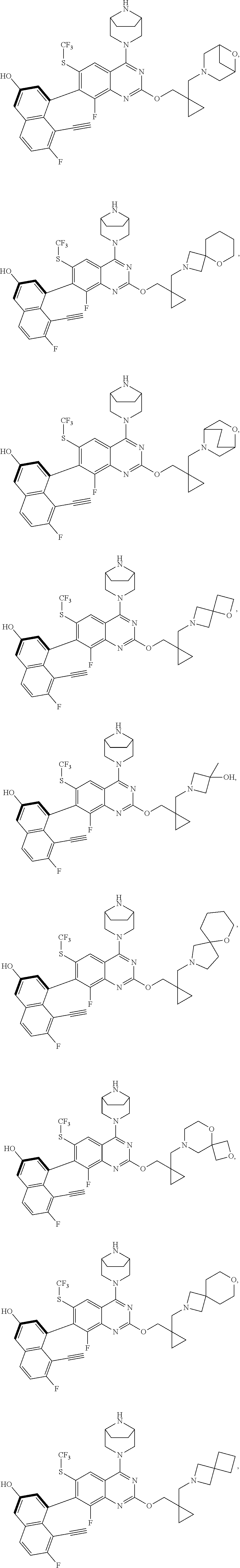

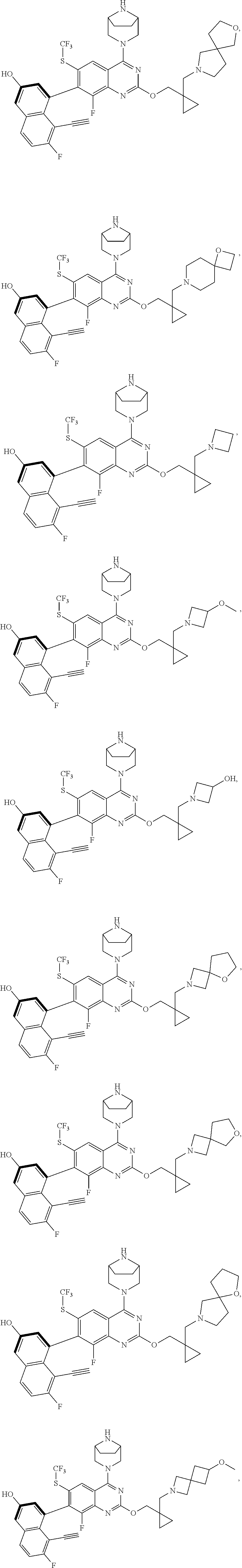

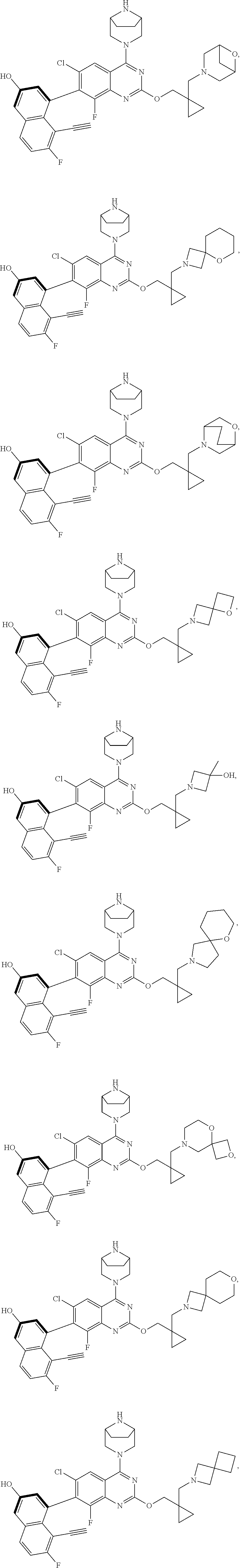

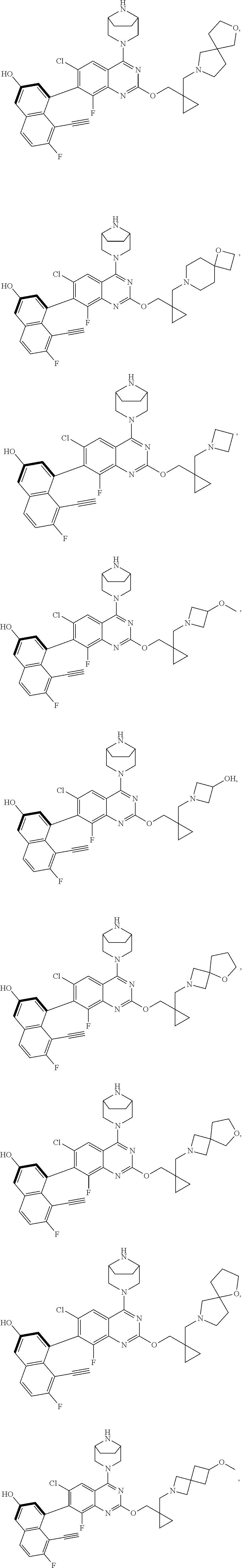

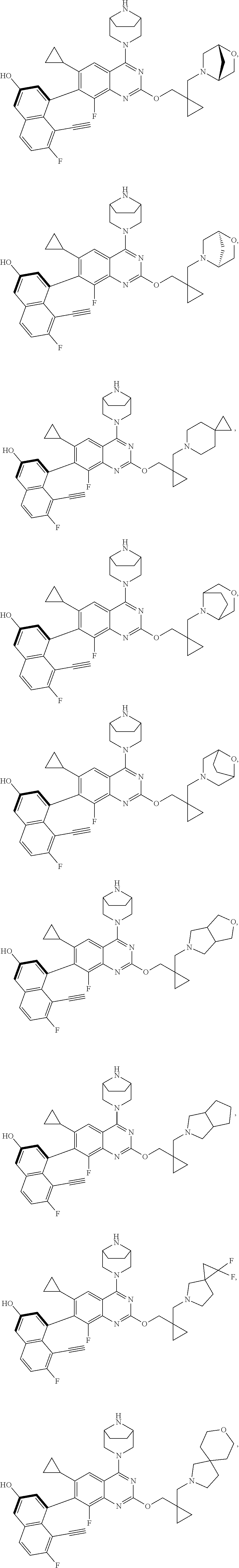

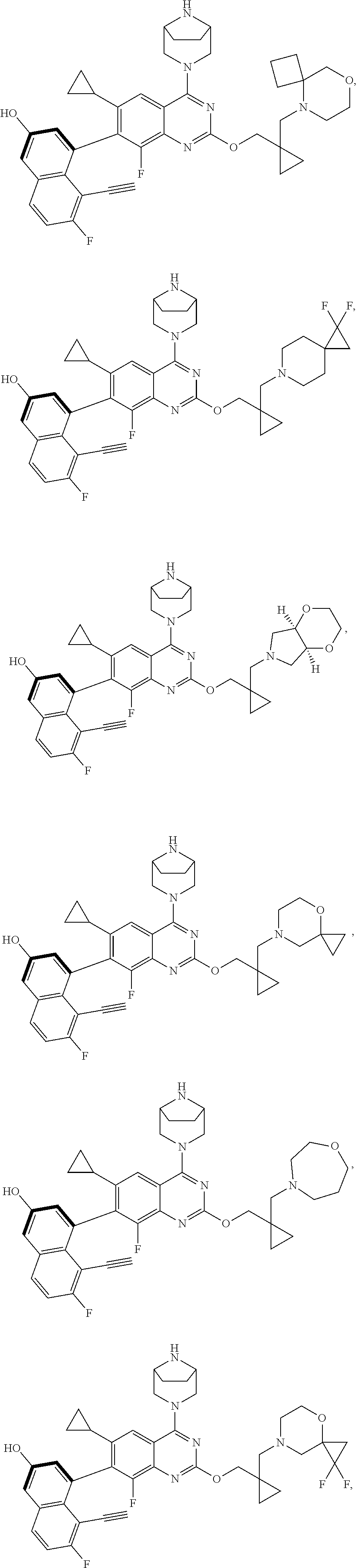

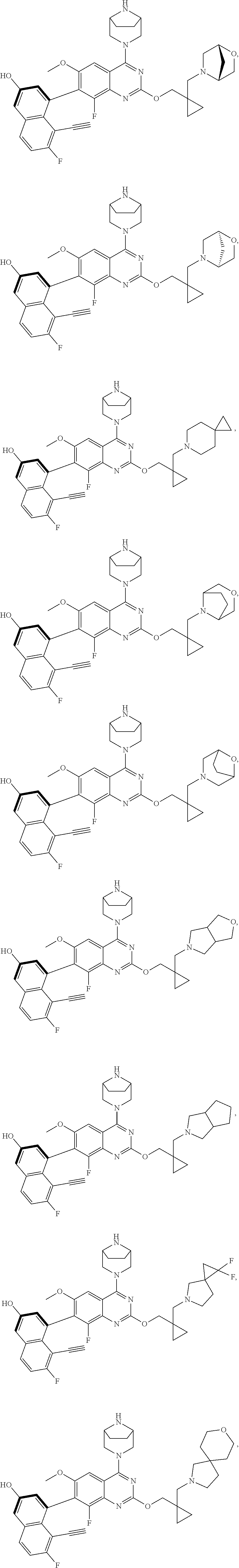

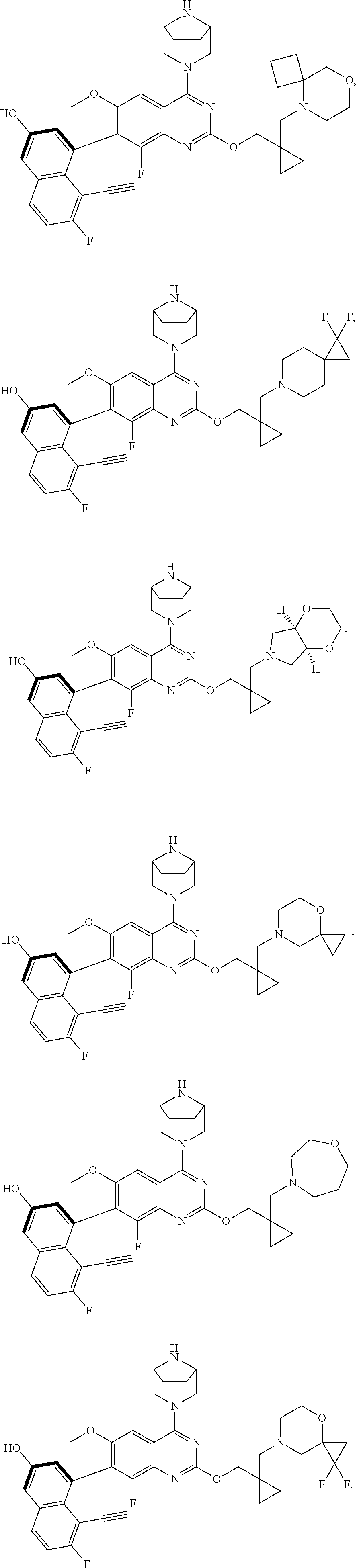

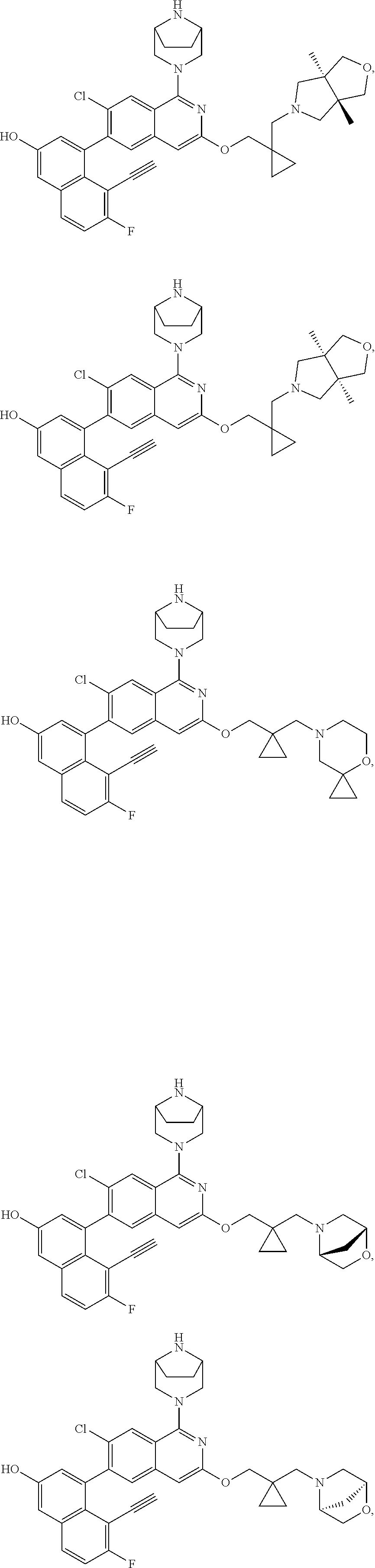 [SMILES: C#Cc1c(F)ccc2cc(O)cc(-c3cc4cc(OCC5(CN6C[C@@]7(C)COC[C@]7(C)C6)CC5)nc(N5CC6CCC(C5)N6)c4cc3Cl)c12 ; C#Cc1c(F)ccc2cc(O)cc(-c3cc4cc(OCC5(CN6C[C@]7(C)COC[C@]7(C)C6)CC5)nc(N5CC6CCC(C5)N6)c4cc3Cl)c12 ; C#Cc1c(F)ccc2cc(O)cc(-c3cc4cc(OCC5(CN6CCOC7(CC7)C6)CC5)nc(N5CC6CCC(C5)N6)c4cc3Cl)c12 ; C#Cc1c(F)ccc2cc(O)cc(-c3cc4cc(OCC5(CN6C[C@H]7C[C@@H]6CO7)CC5)nc(N5CC6CCC(C5)N6)c4cc3Cl)c12 ; C#Cc1c(F)ccc2cc(O)cc(-c3cc4cc(OCC5(CN6C[C@@H]7C[C@H]6CO7)CC5)nc(N5CC6CCC(C5)N6)c4cc3Cl)c12]

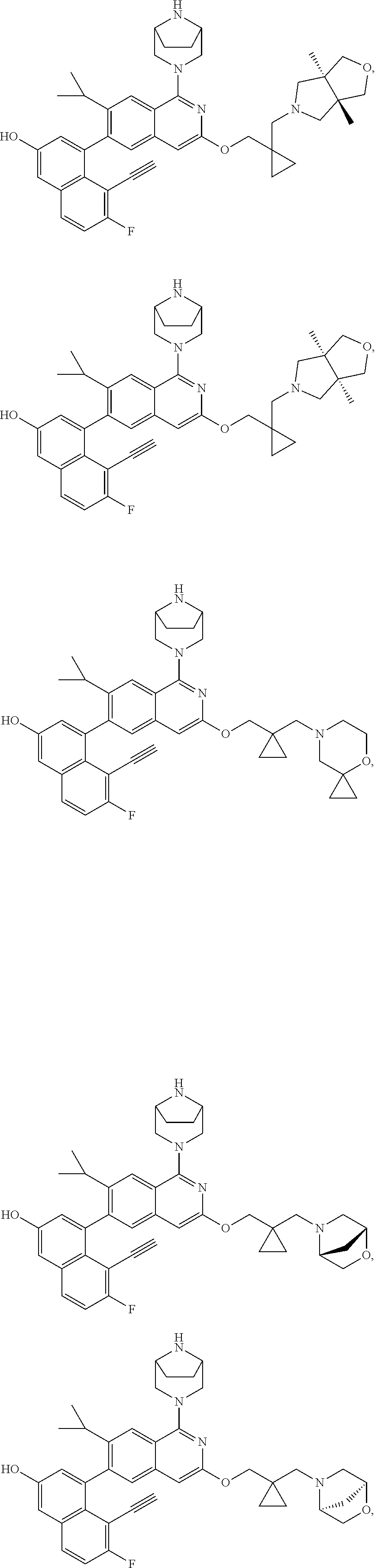 [SMILES: C#Cc1c(F)ccc2cc(O)cc(-c3cc4cc(OCC5(CN6C[C@@]7(C)COC[C@]7(C)C6)CC5)nc(N5CC6CCC(C5)N6)c4cc3C(C)C)c12 ; C#Cc1c(F)ccc2cc(O)cc(-c3cc4cc(OCC5(CN6C[C@]7(C)COC[C@]7(C)C6)CC5)nc(N5CC6CCC(C5)N6)c4cc3C(C)C)c12 ; C#Cc1c(F)ccc2cc(O)cc(-c3cc4cc(OCC5(CN6CCOC7(CC7)C6)CC5)nc(N5CC6CCC(C5)N6)c4cc3C(C)C)c12 ; C#Cc1c(F)ccc2cc(O)cc(-c3cc4cc(OCC5(CN6C[C@H]7C[C@@H]6CO7)CC5)nc(N5CC6CCC(C5)N6)c4cc3C(C)C)c12 ; C#Cc1c(F)ccc2cc(O)cc(-c3cc4cc(OCC5(CN6C[C@@H]7C[C@H]6CO7)CC5)nc(N5CC6CCC(C5)N6)c4cc3C(C)C)c12]

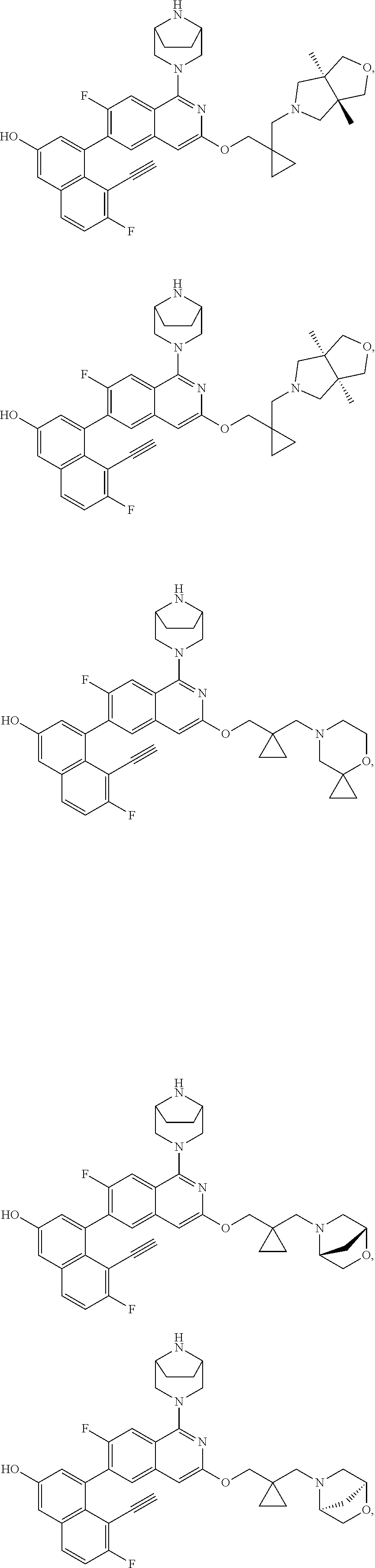 [SMILES: C#Cc1c(F)ccc2cc(O)cc(-c3cc4cc(OCC5(CN6C[C@@]7(C)COC[C@]7(C)C6)CC5)nc(N5CC6CCC(C5)N6)c4cc3F)c12 ; C#Cc1c(F)ccc2cc(O)cc(-c3cc4cc(OCC5(CN6C[C@]7(C)COC[C@]7(C)C6)CC5)nc(N5CC6CCC(C5)N6)c4cc3F)c12 ; C#Cc1c(F)ccc2cc(O)cc(-c3cc4cc(OCC5(CN6CCOC7(CC7)C6)CC5)nc(N5CC6CCC(C5)N6)c4cc3F)c12 ; C#Cc1c(F)ccc2cc(O)cc(-c3cc4cc(OCC5(CN6C[C@H]7C[C@@H]6CO7)CC5)nc(N5CC6CCC(C5)N6)c4cc3F)c12 ; C#Cc1c(F)ccc2cc(O)cc(-c3cc4cc(OCC5(CN6C[C@@H]7C[C@H]6CO7)CC5)nc(N5CC6CCC(C5)N6)c4cc3F)c12]

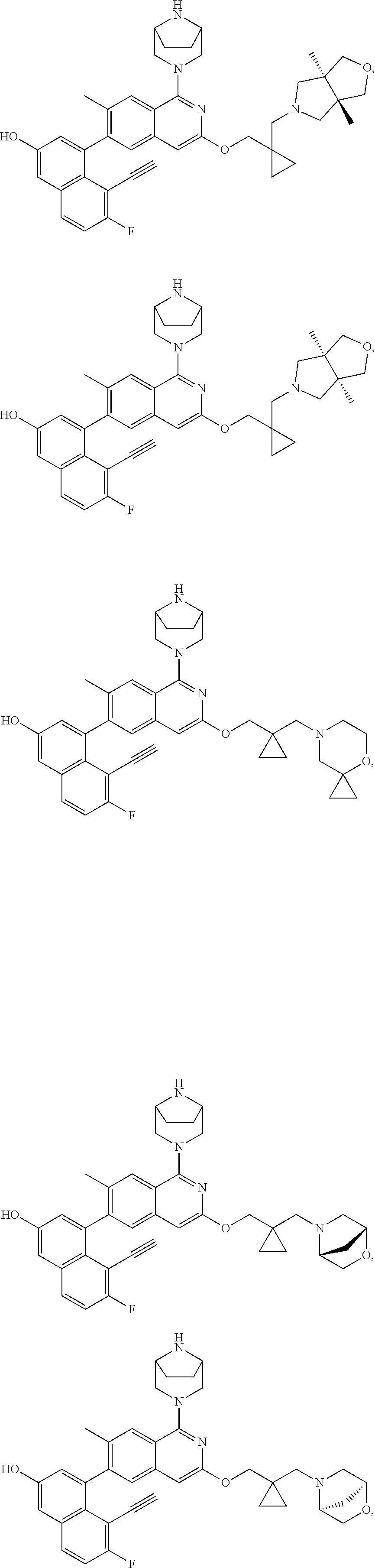 [SMILES: C#Cc1c(F)ccc2cc(O)cc(-c3cc4cc(OCC5(CN6C[C@@]7(C)COC[C@]7(C)C6)CC5)nc(N5CC6CCC(C5)N6)c4cc3C)c12 ; C#Cc1c(F)ccc2cc(O)cc(-c3cc4cc(OCC5(CN6C[C@]7(C)COC[C@]7(C)C6)CC5)nc(N5CC6CCC(C5)N6)c4cc3C)c12 ; C#Cc1c(F)ccc2cc(O)cc(-c3cc4cc(OCC5(CN6CCOC7(CC7)C6)CC5)nc(N5CC6CCC(C5)N6)c4cc3C)c12 ; C#Cc1c(F)ccc2cc(O)cc(-c3cc4cc(OCC5(CN6C[C@H]7C[C@@H]6CO7)CC5)nc(N5CC6CCC(C5)N6)c4cc3C)c12 ; C#Cc1c(F)ccc2cc(O)cc(-c3cc4cc(OCC5(CN6C[C@@H]7C[C@H]6CO7)CC5)nc(N5CC6CCC(C5)N6)c4cc3C)c12]

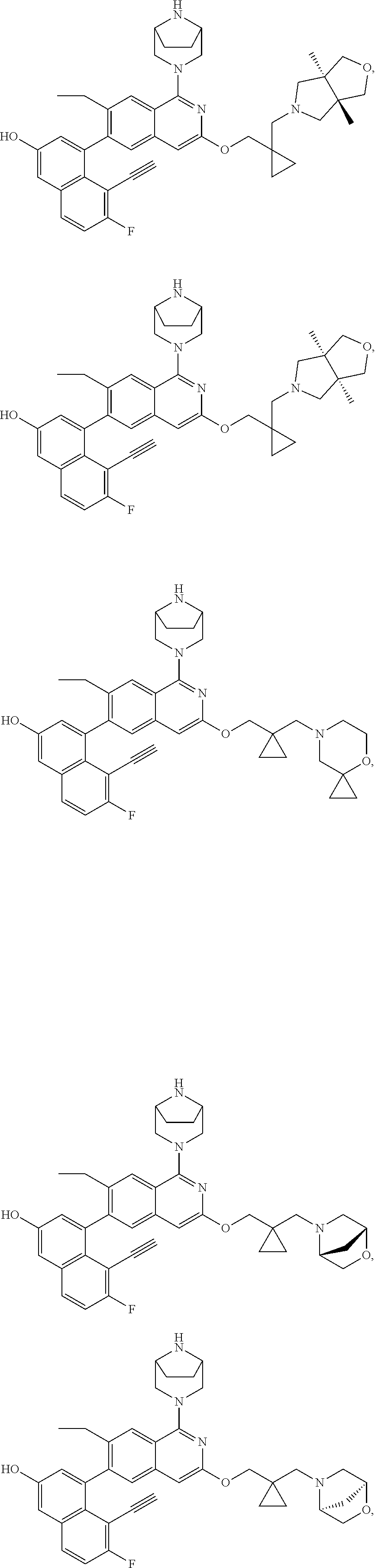 [SMILES: C#Cc1c(F)ccc2cc(O)cc(-c3cc4cc(OCC5(CN6C[C@@]7(C)COC[C@]7(C)C6)CC5)nc(N5CC6CCC(C5)N6)c4cc3CC)c12 ; C#Cc1c(F)ccc2cc(O)cc(-c3cc4cc(OCC5(CN6C[C@]7(C)COC[C@]7(C)C6)CC5)nc(N5CC6CCC(C5)N6)c4cc3CC)c12 ; C#Cc1c(F)ccc2cc(O)cc(-c3cc4cc(OCC5(CN6CCOC7(CC7)C6)CC5)nc(N5CC6CCC(C5)N6)c4cc3CC)c12 ; C#Cc1c(F)ccc2cc(O)cc(-c3cc4cc(OCC5(CN6C[C@H]7C[C@@H]6CO7)CC5)nc(N5CC6CCC(C5)N6)c4cc3CC)c12 ; C#Cc1c(F)ccc2cc(O)cc(-c3cc4cc(OCC5(CN6C[C@@H]7C[C@H]6CO7)CC5)nc(N5CC6CCC(C5)N6)c4cc3CC)c12]

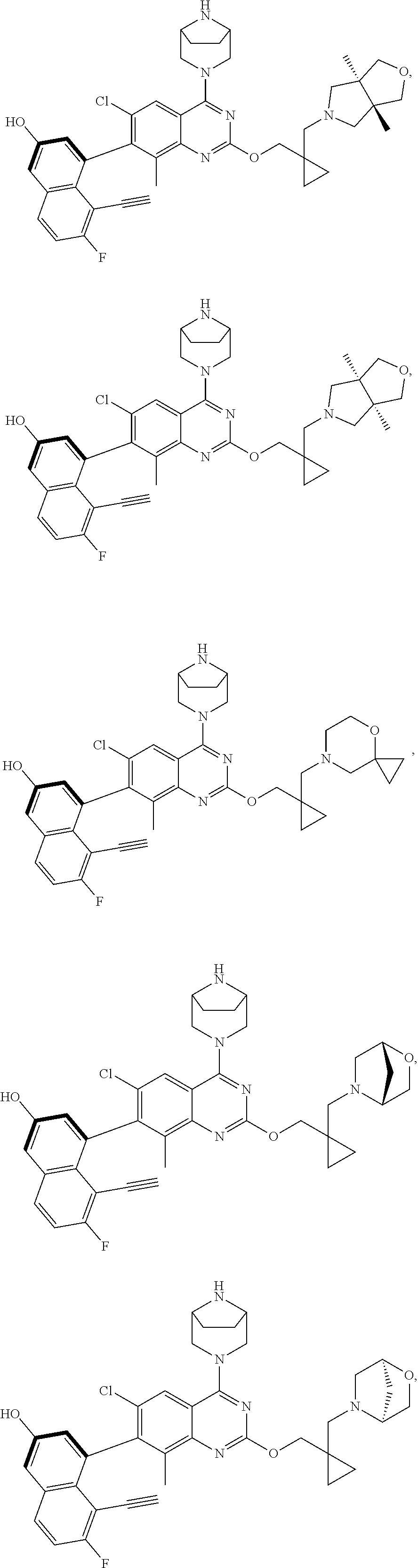

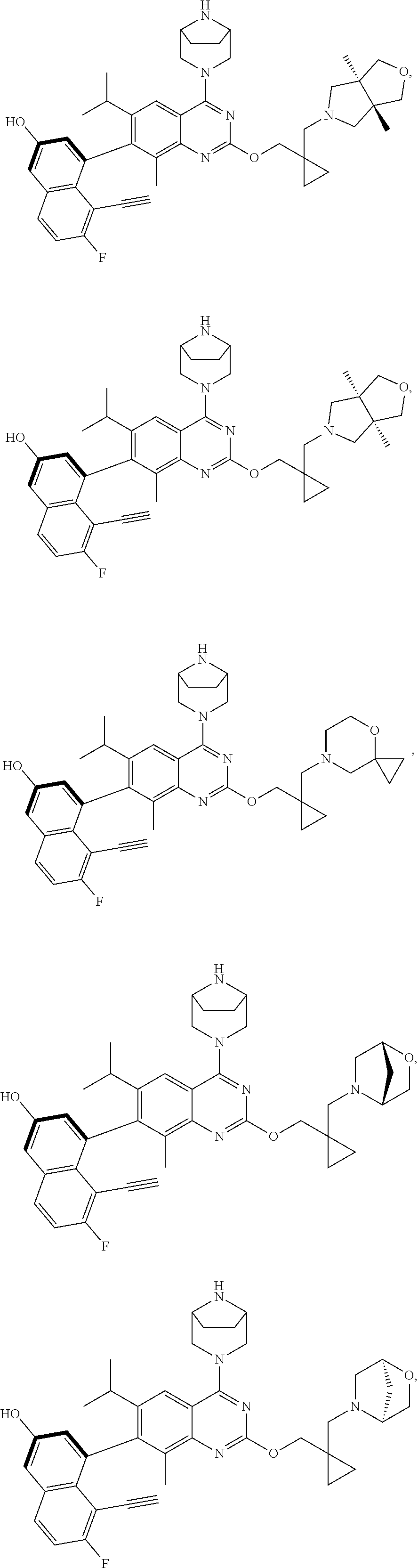

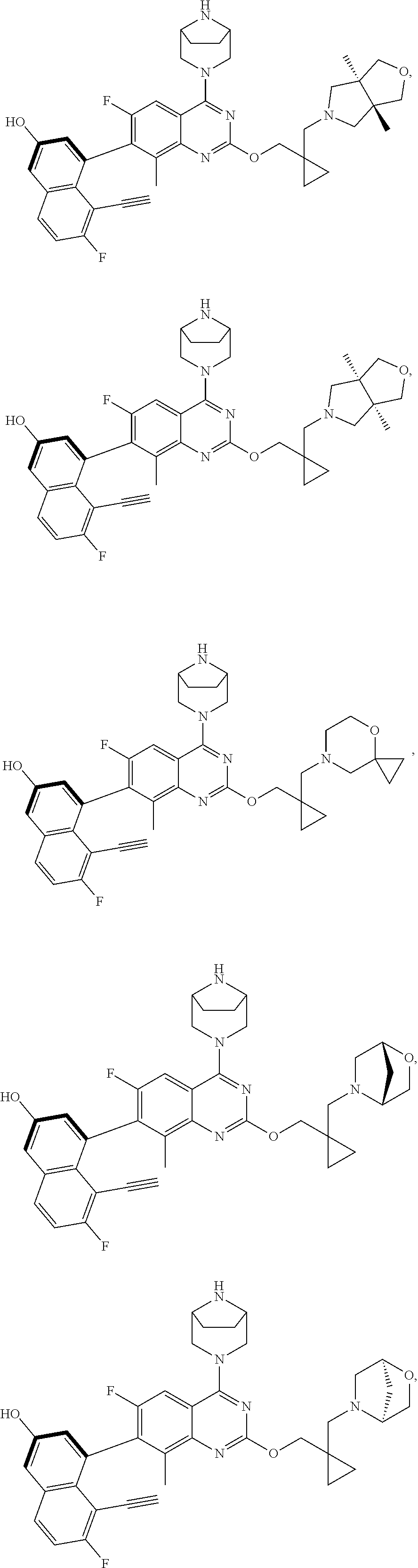

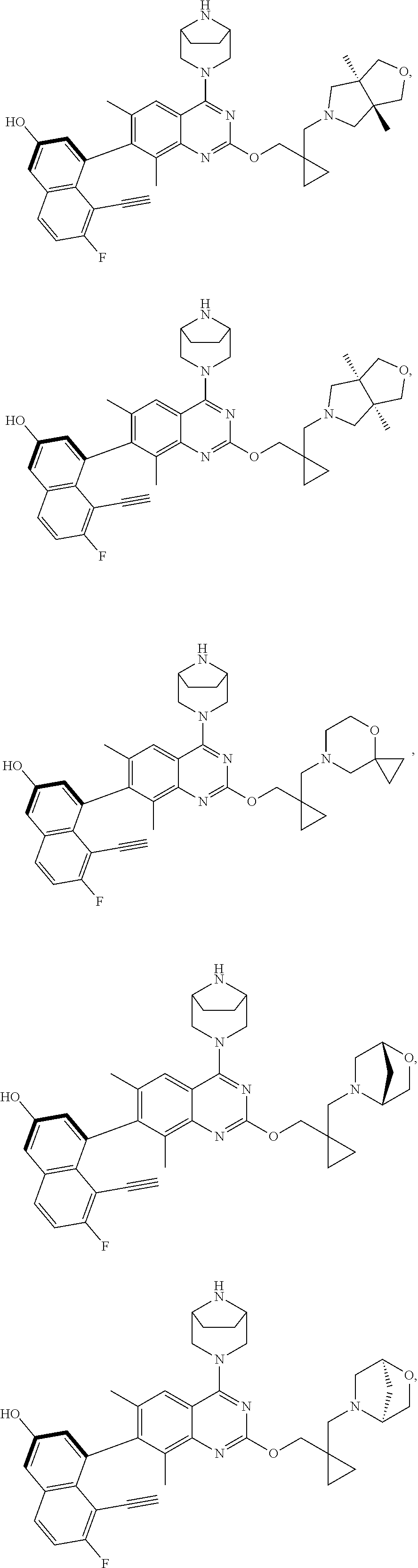

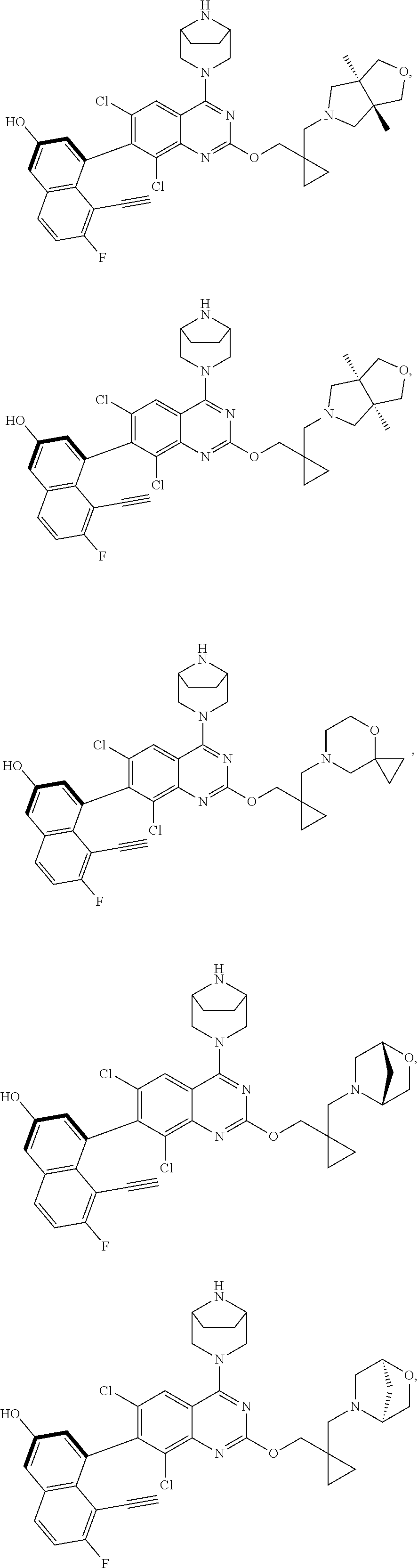

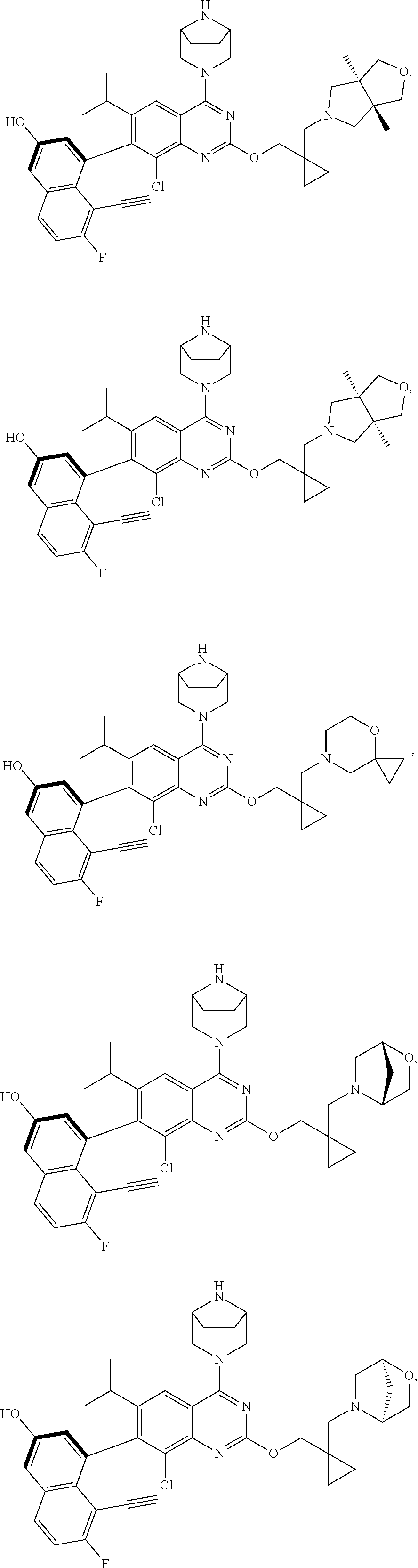

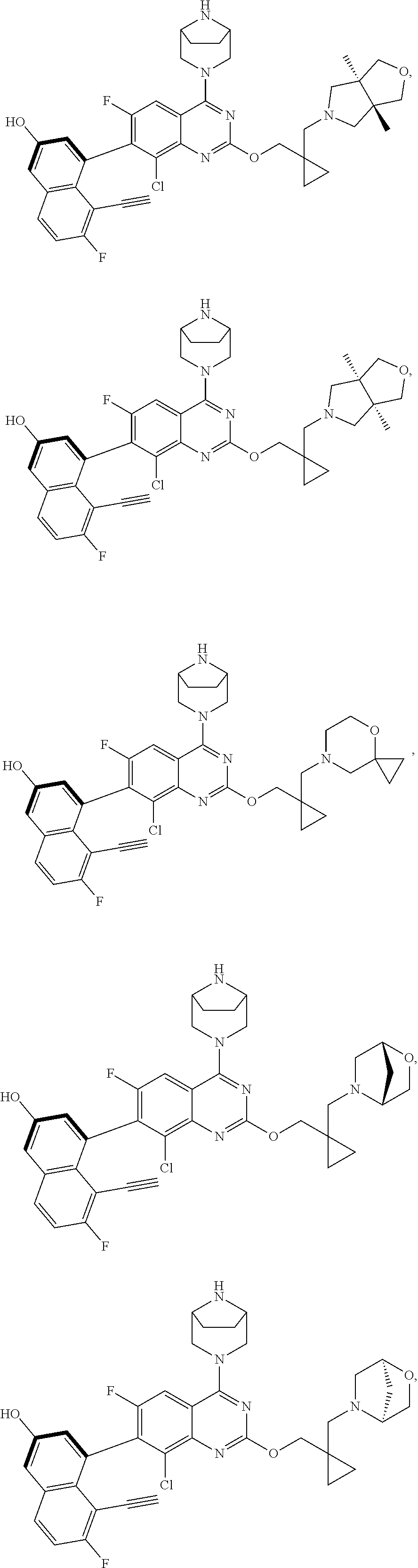

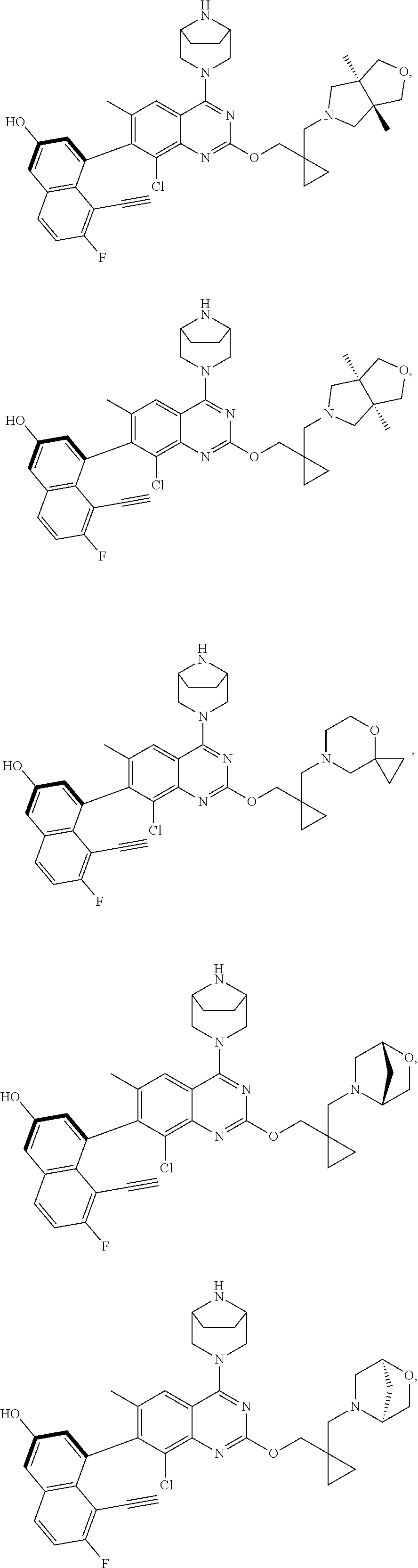

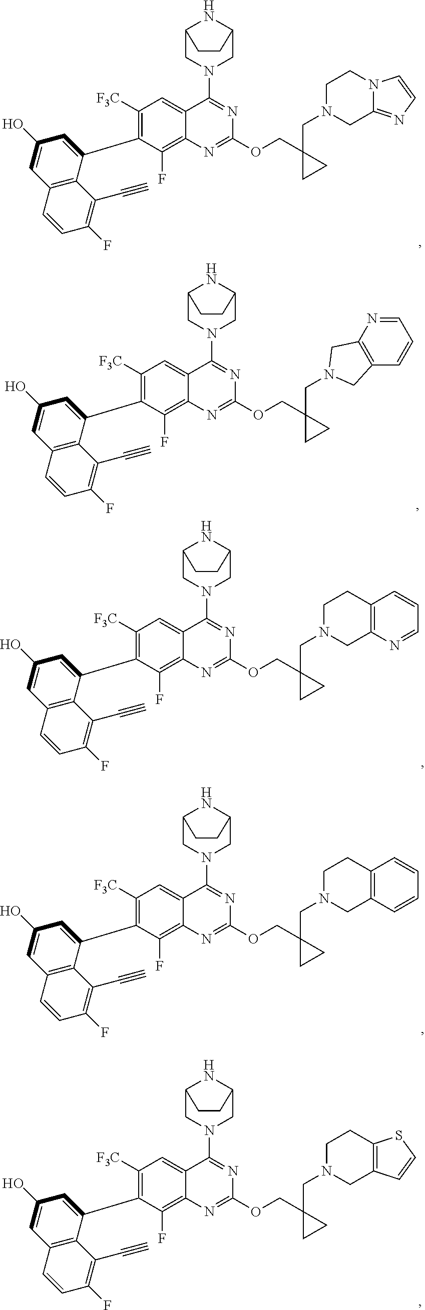

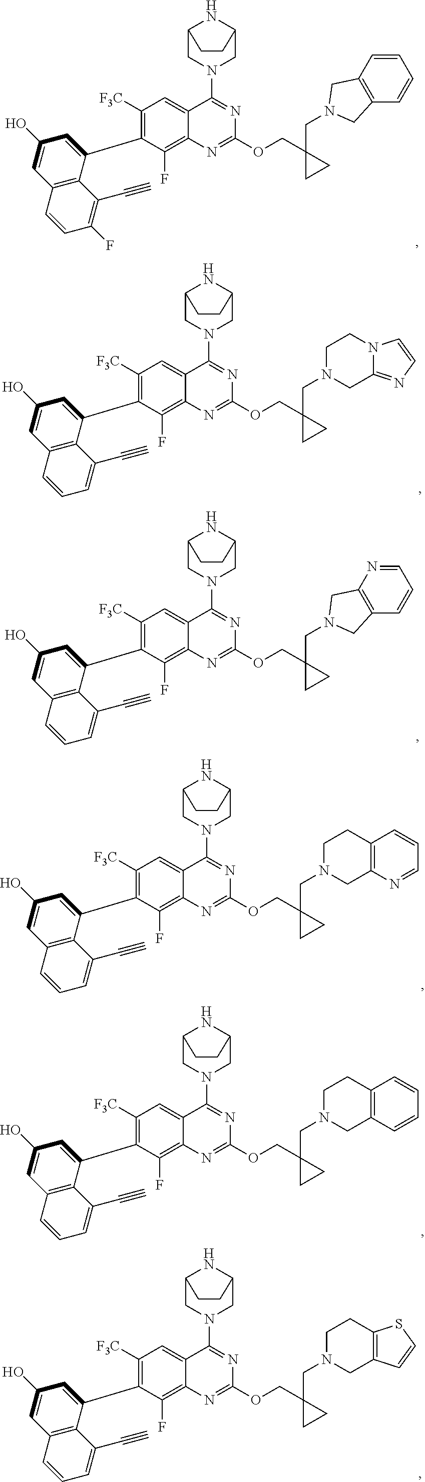

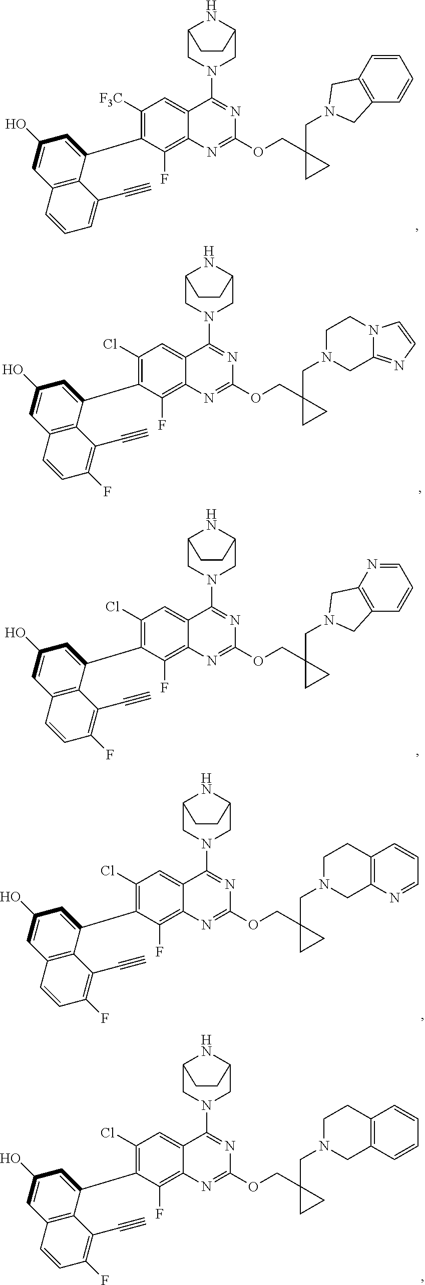

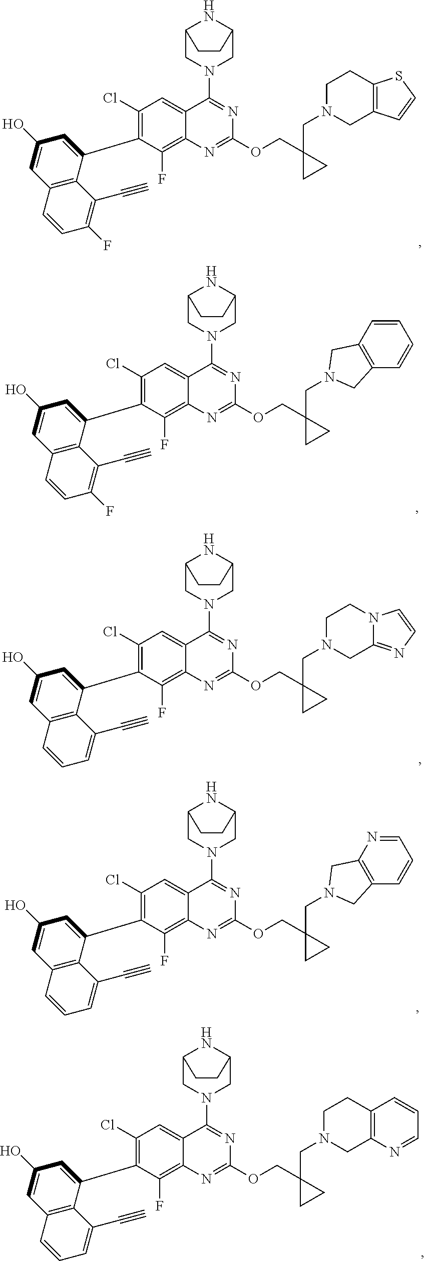

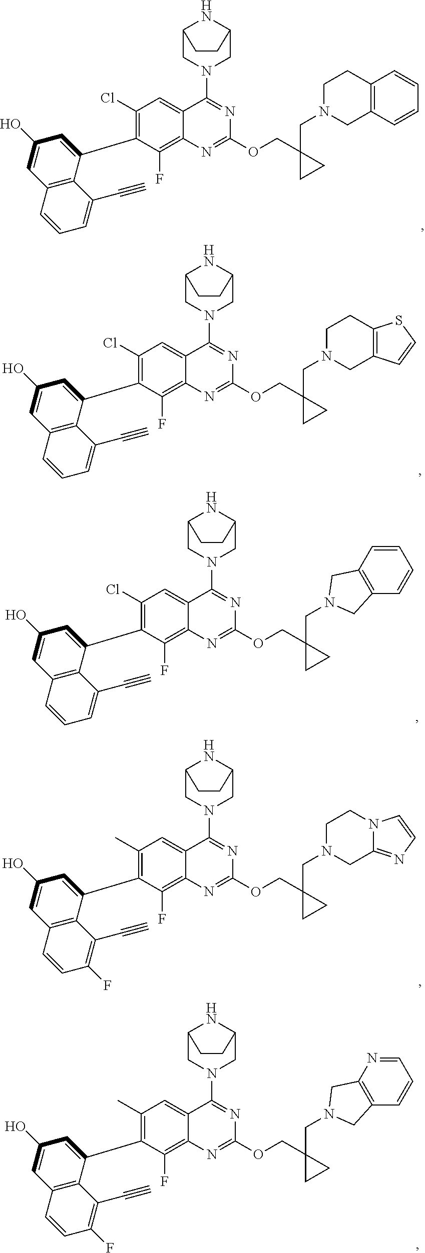

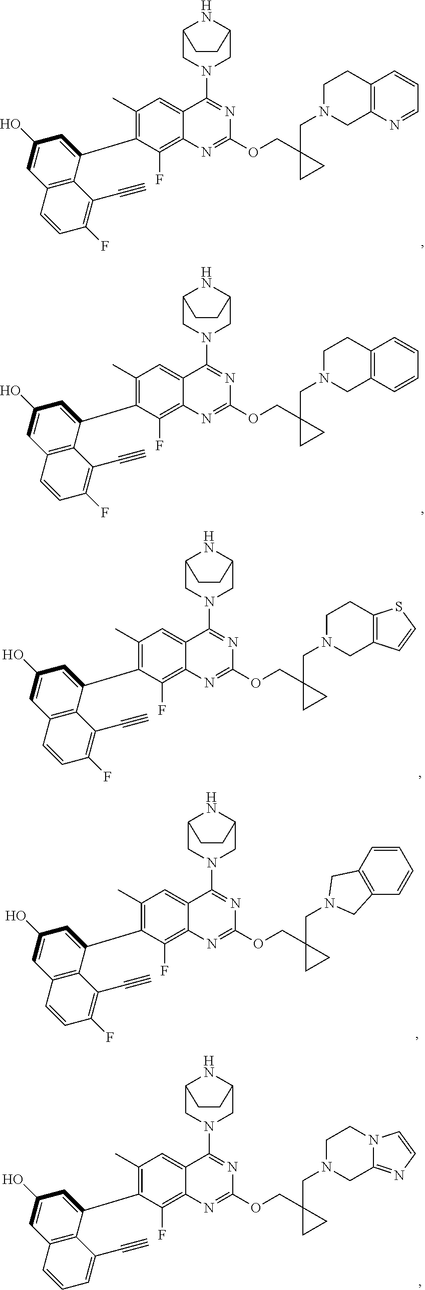

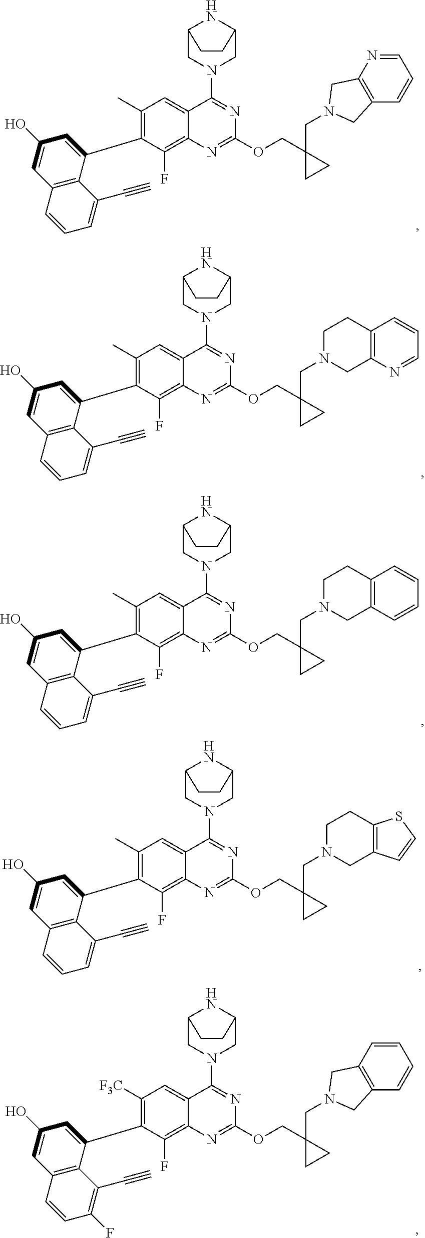

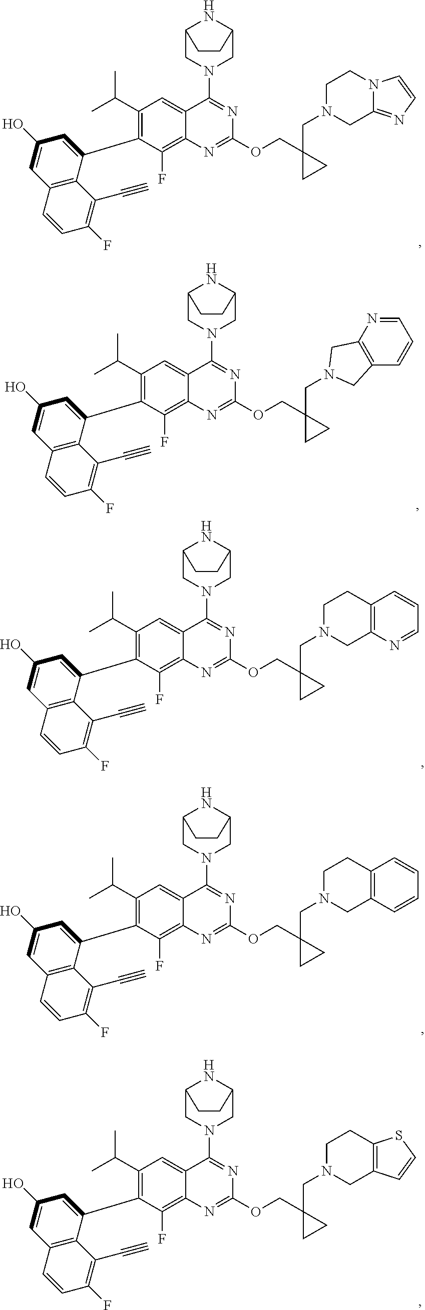

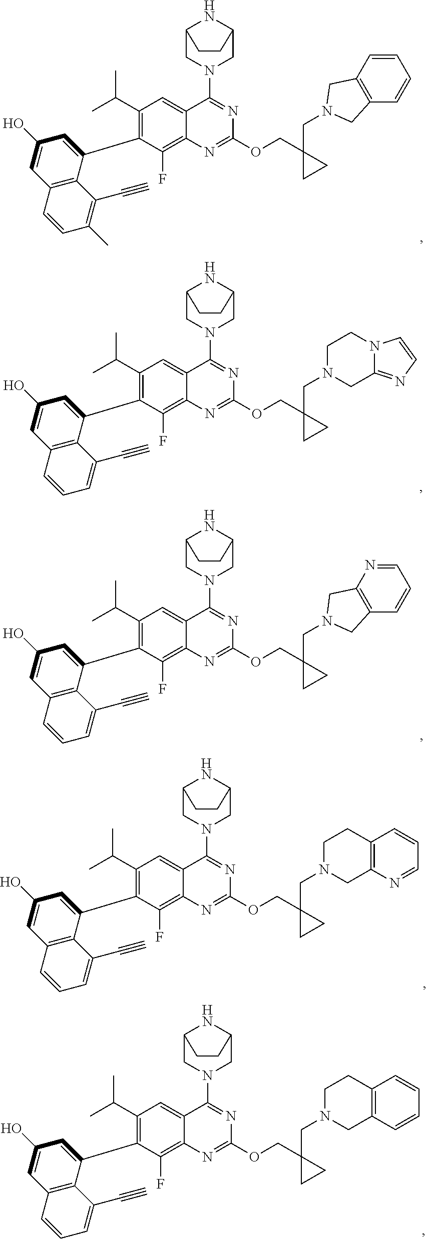

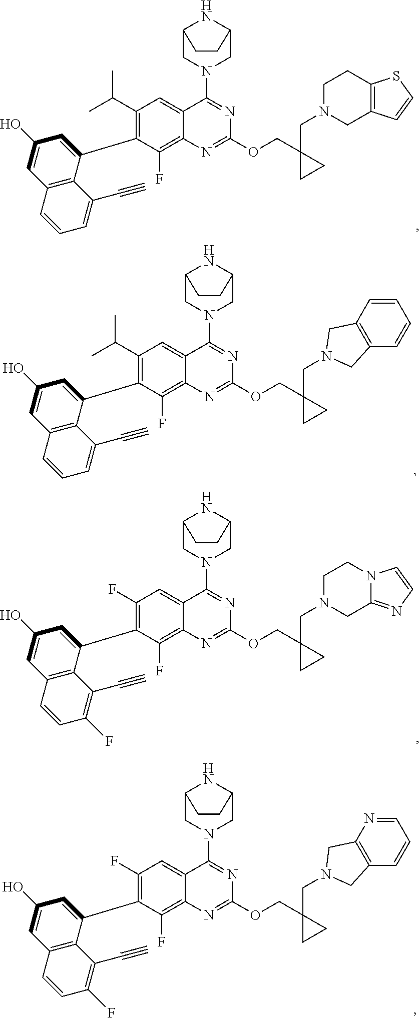

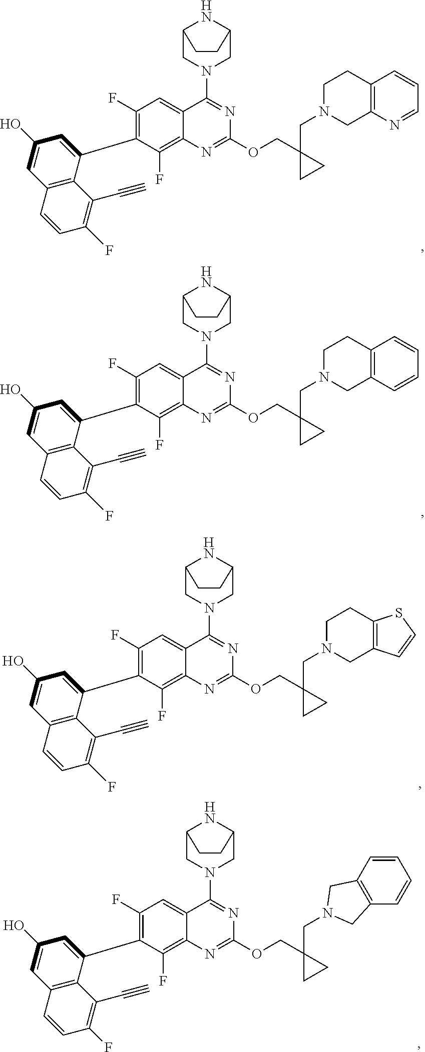

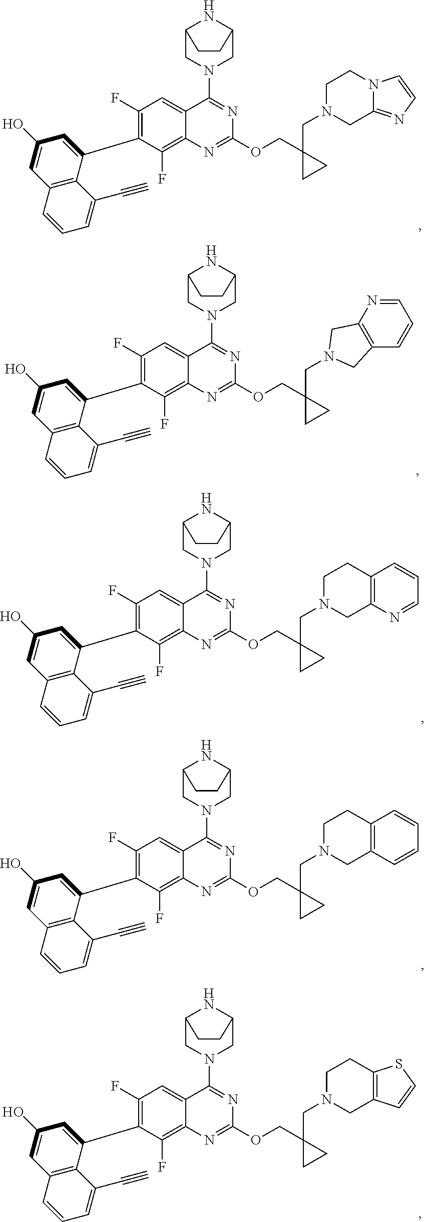

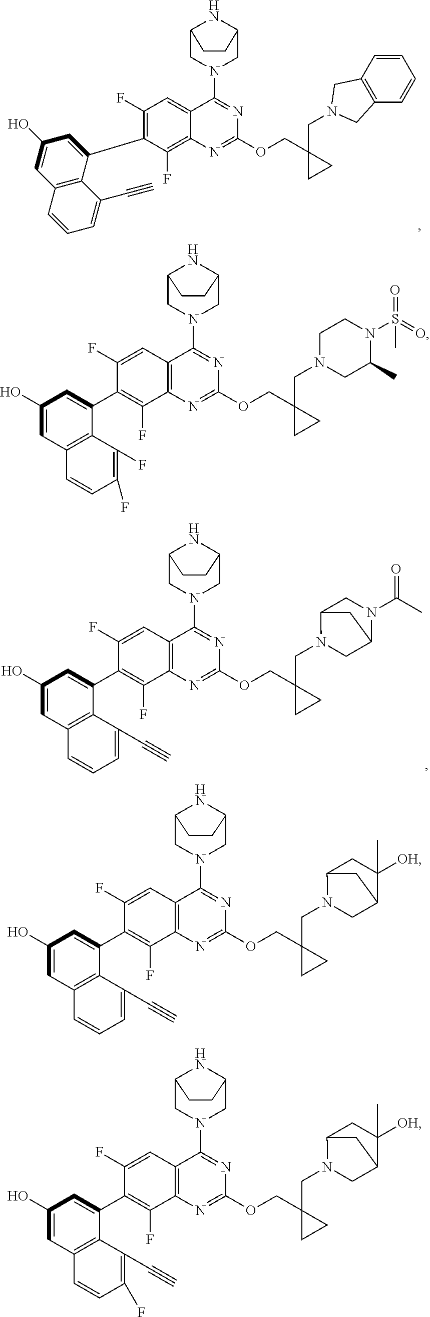

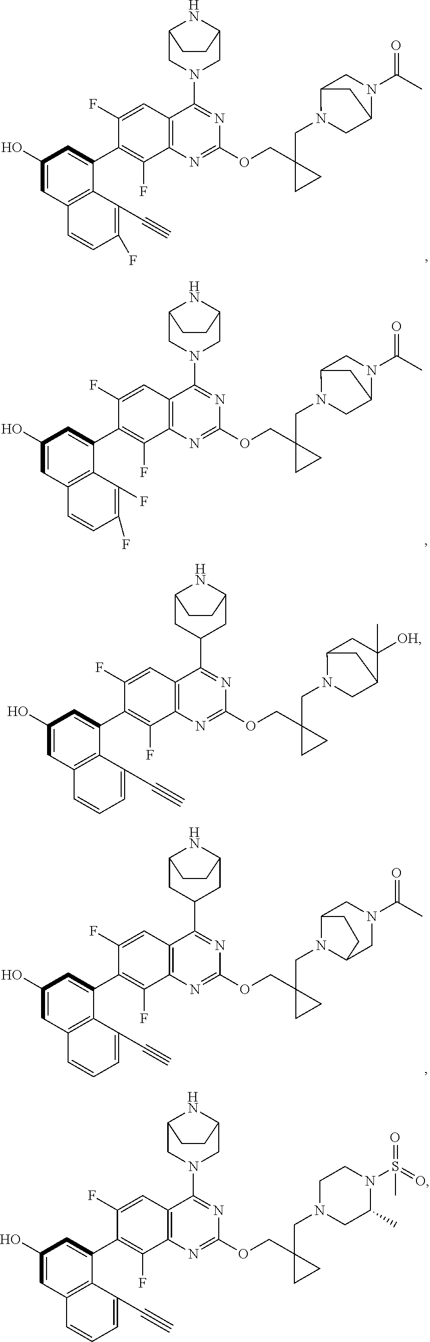

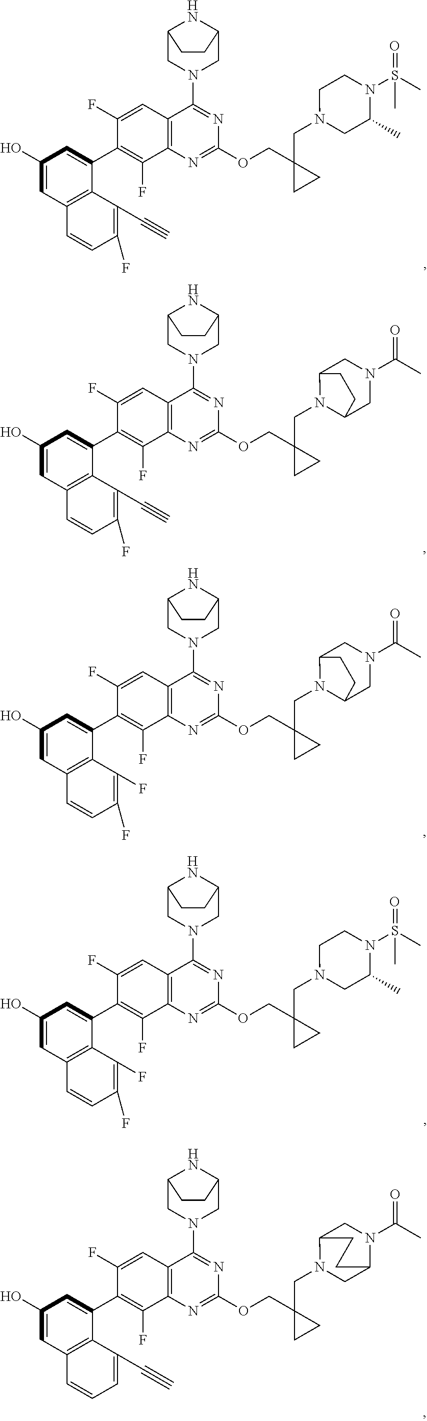

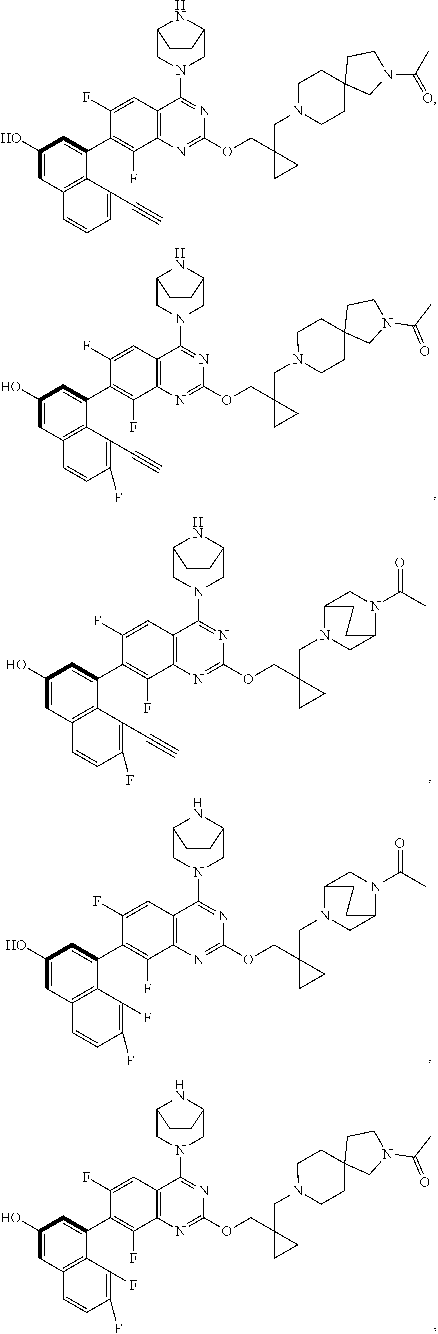

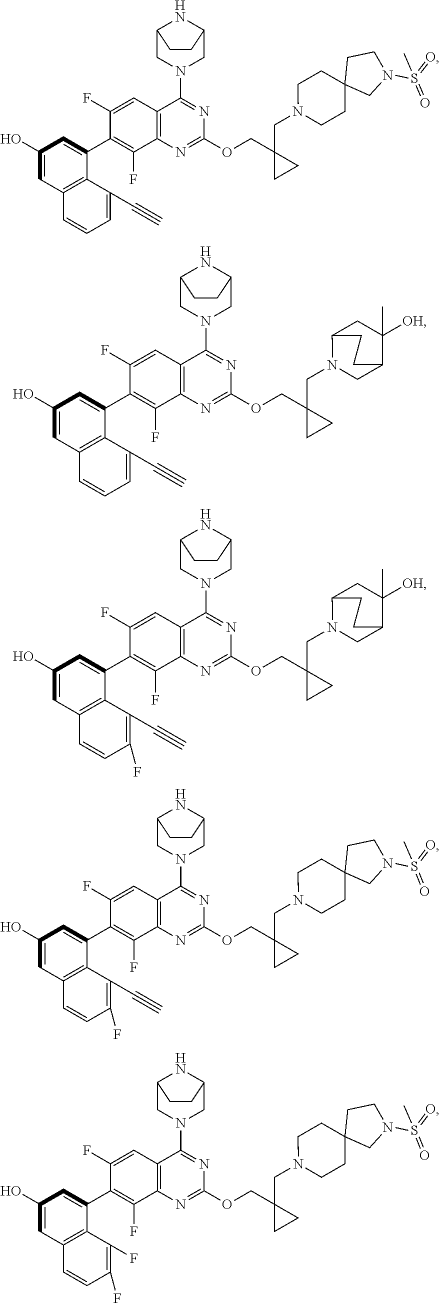

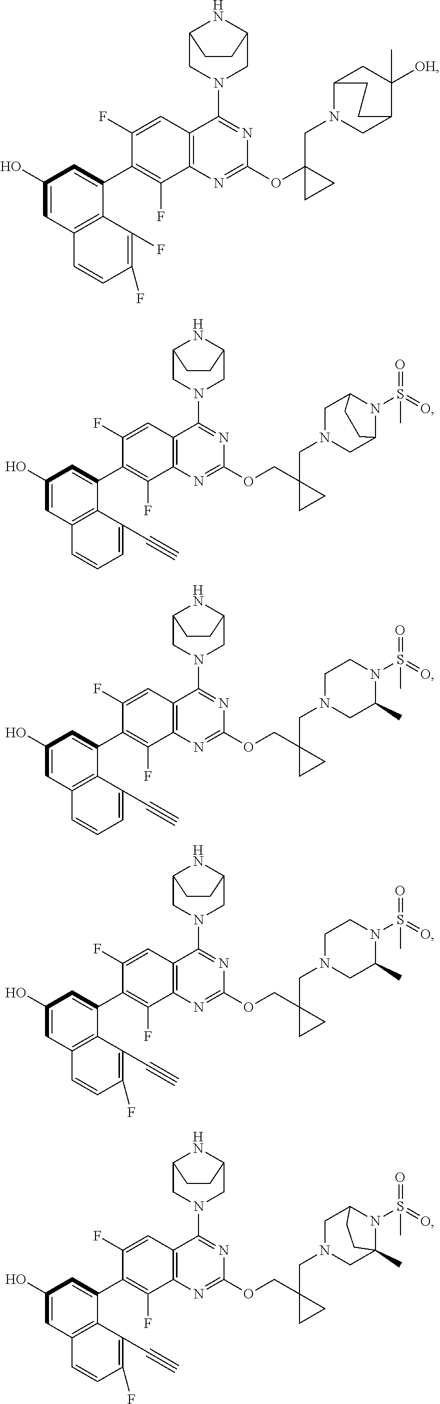

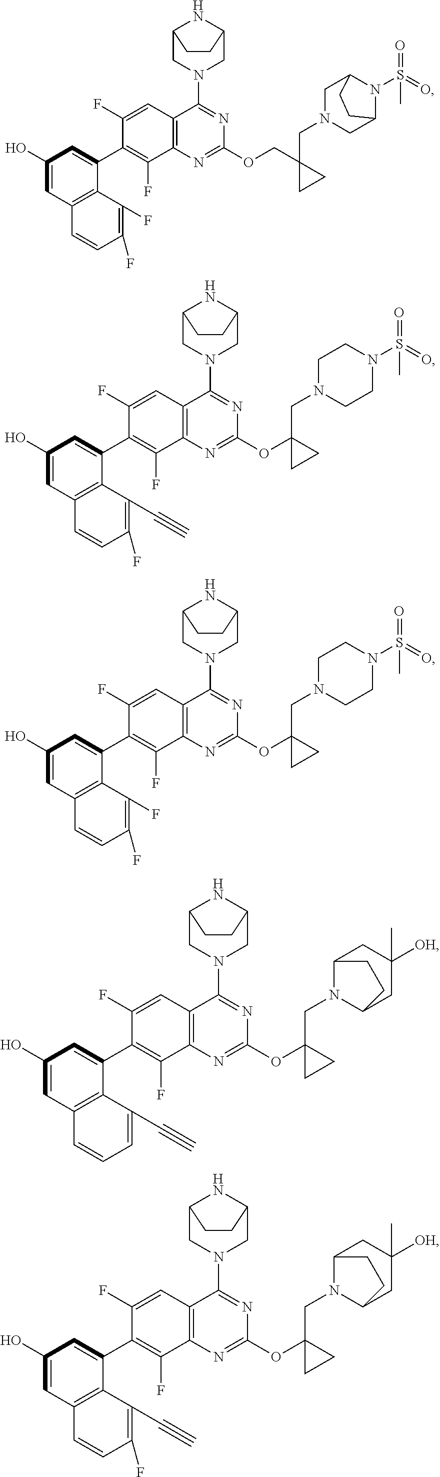

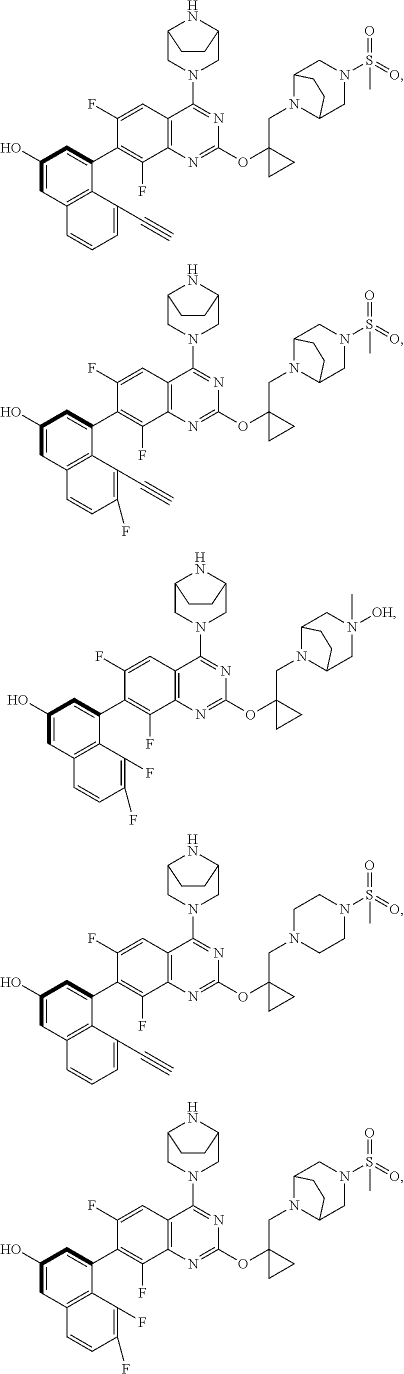

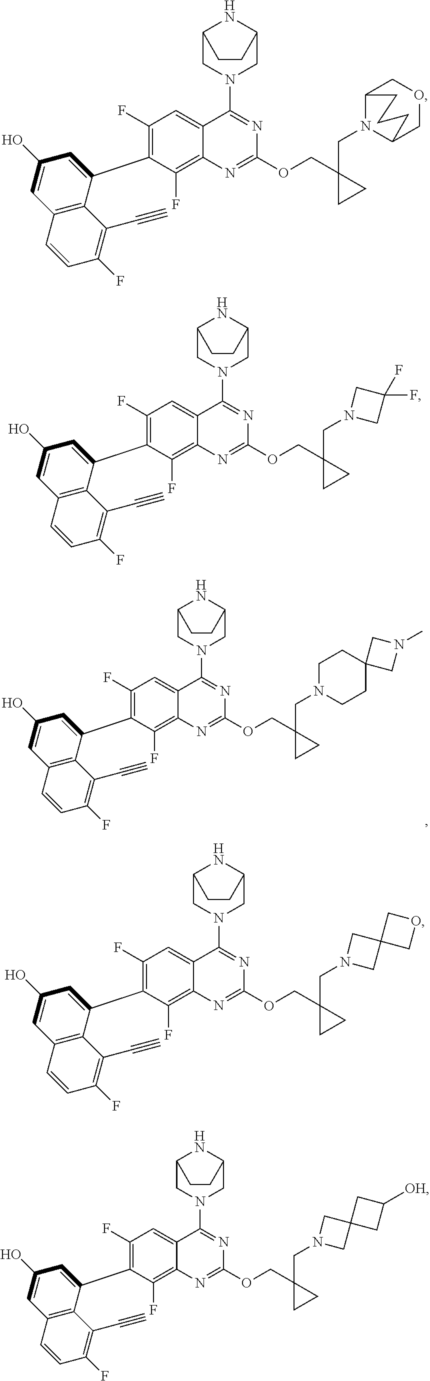

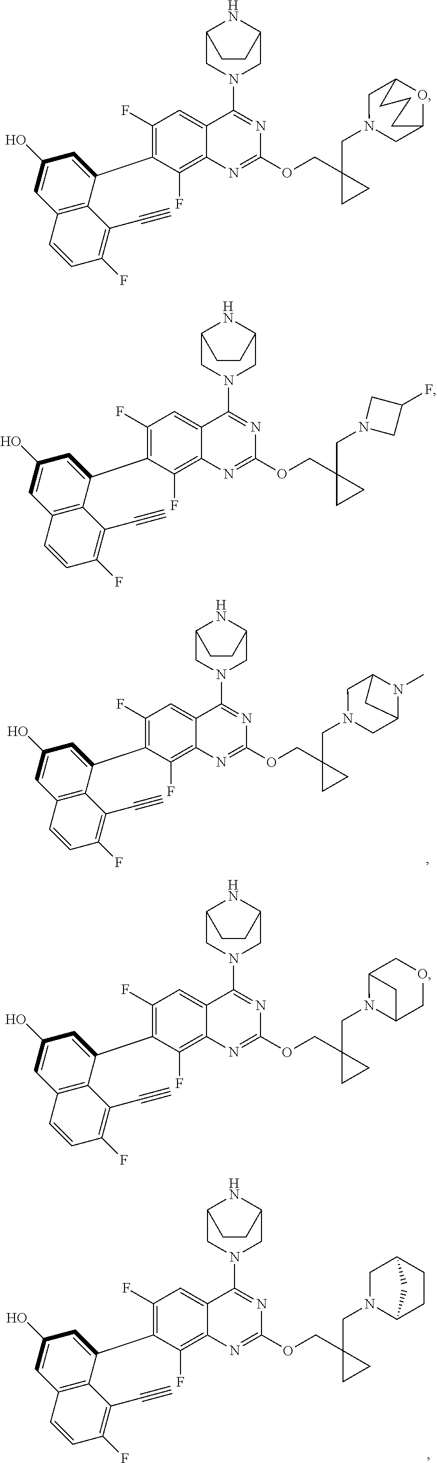

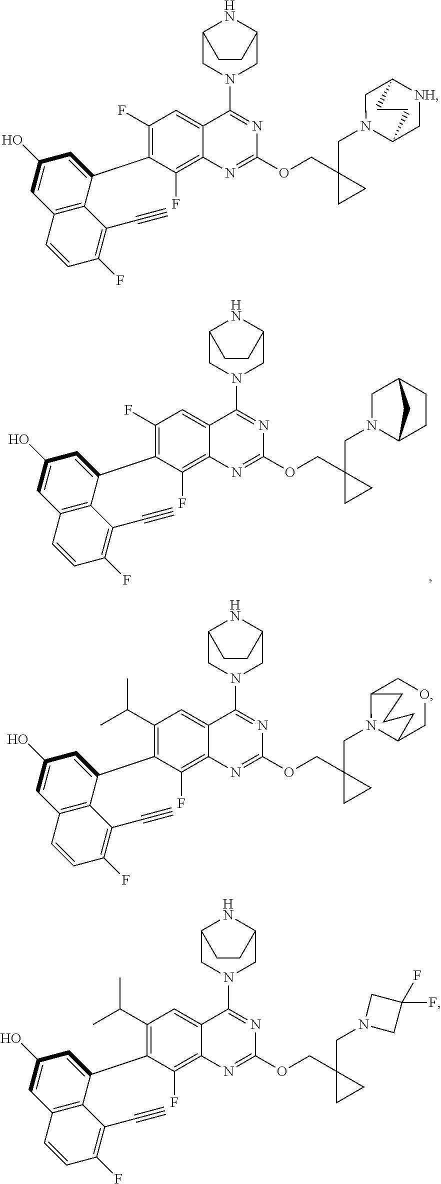

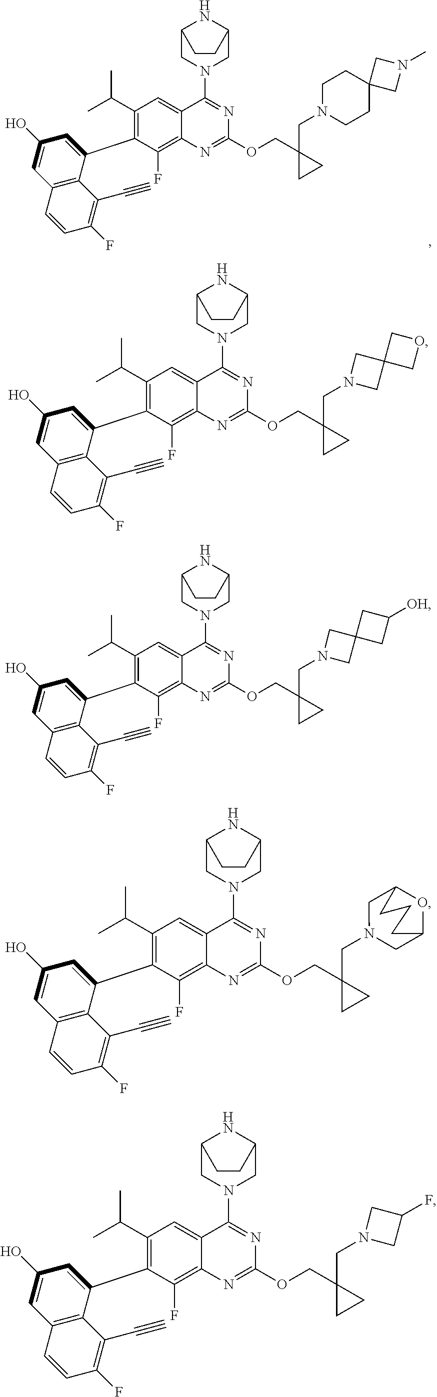

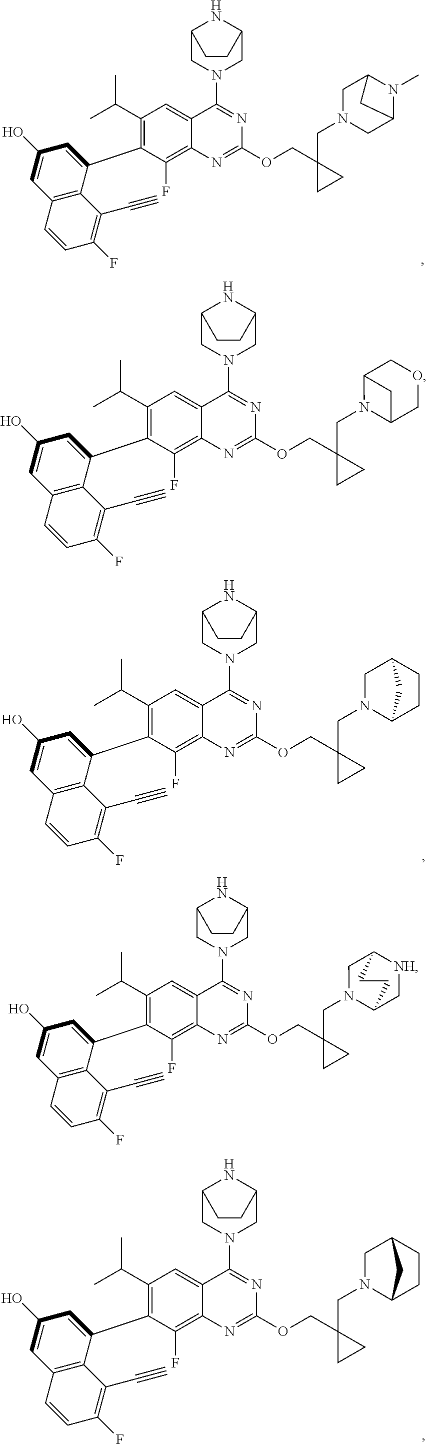

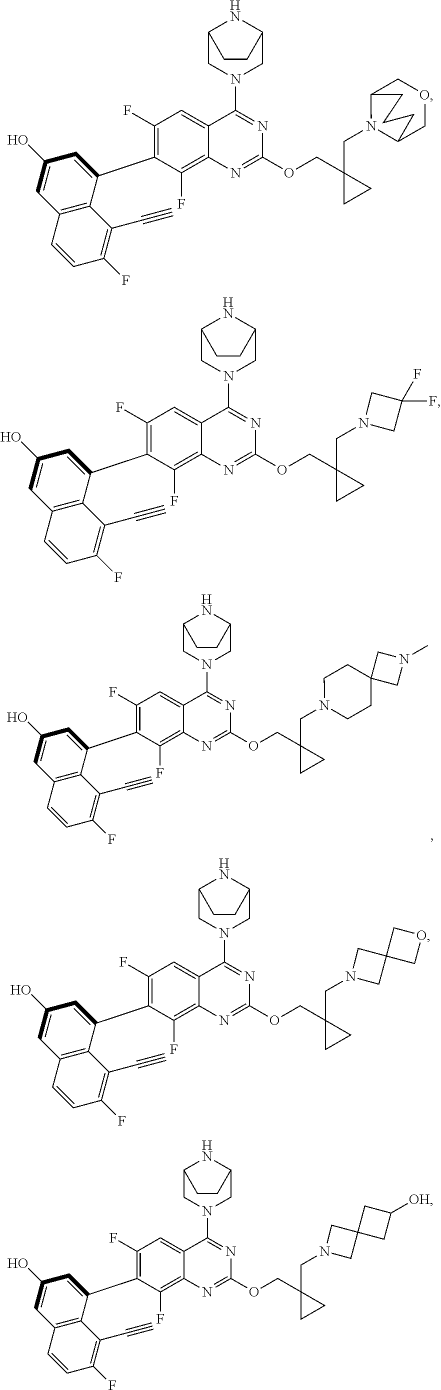

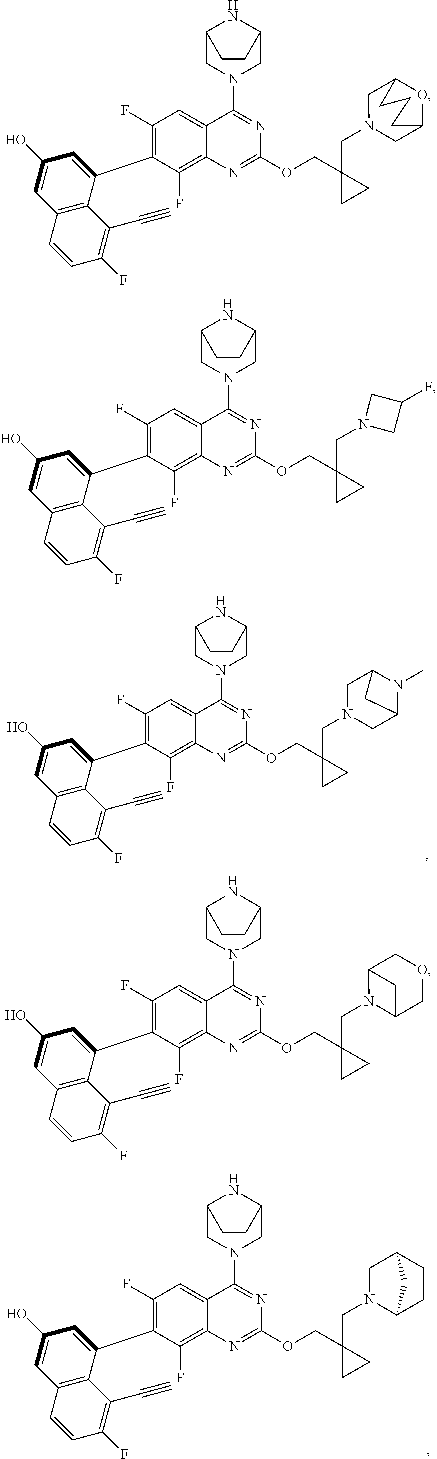

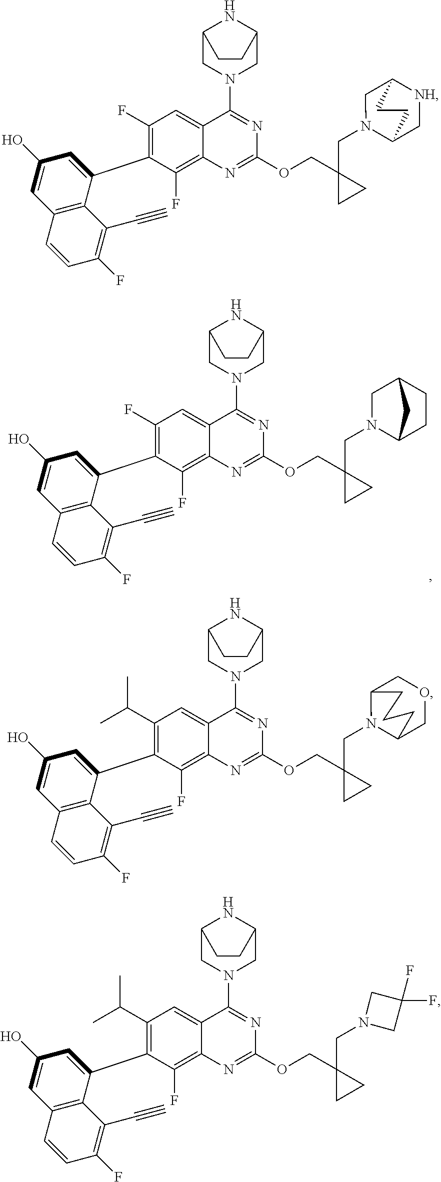

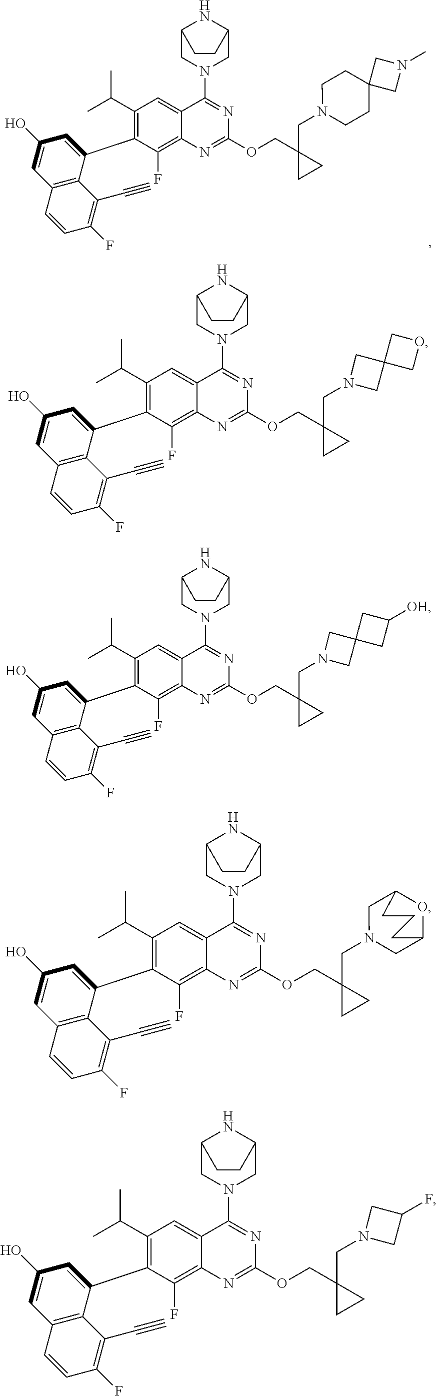

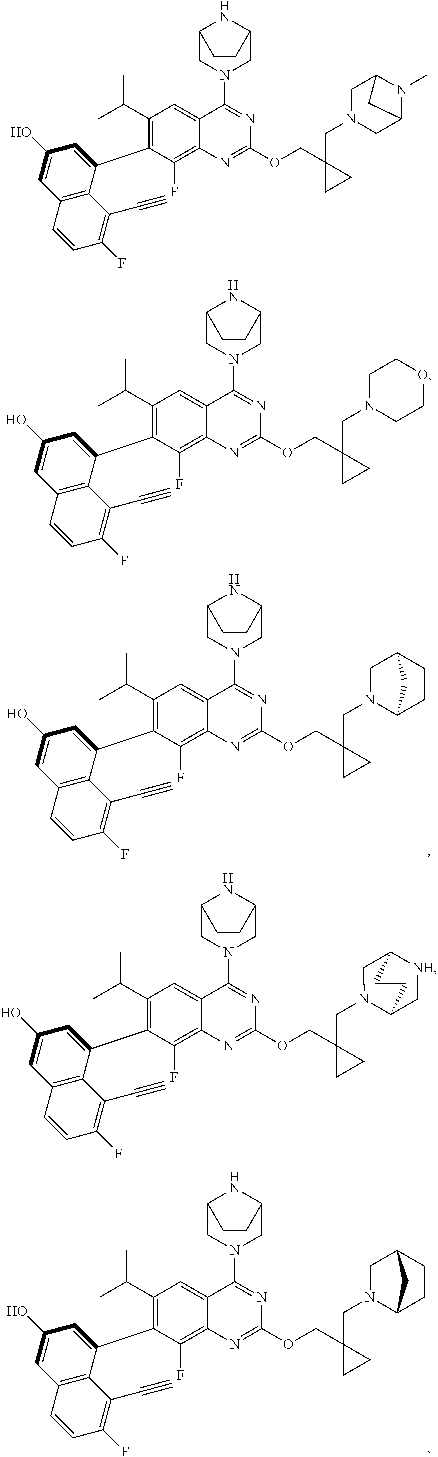

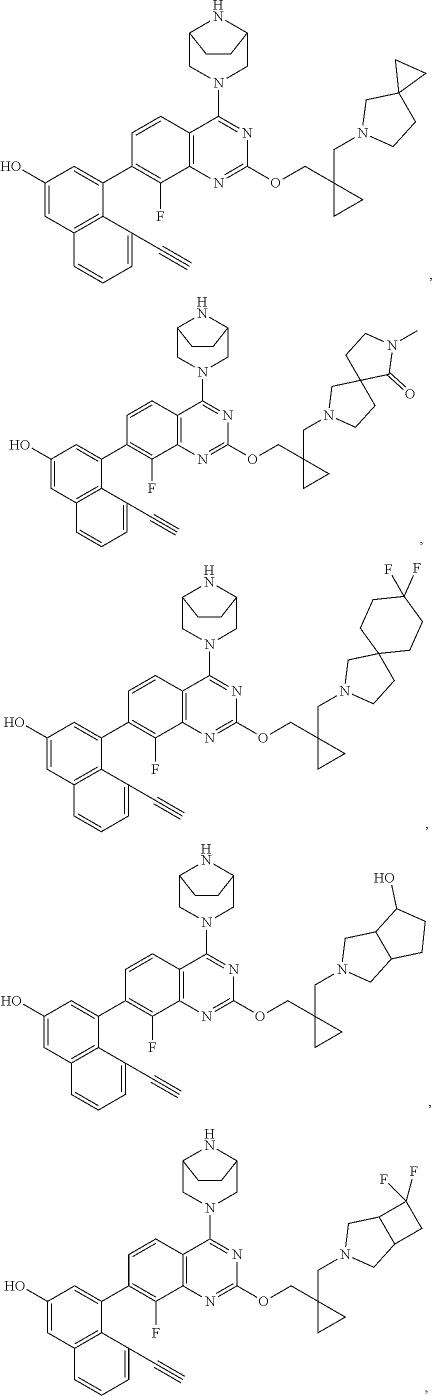 [SMILES: C#Cc1cccc2cc(O)cc(-c3ccc4c(N5CC6CCC(C5)N6)nc(OCC5(CN6CCC7(CC7)C6)CC5)nc4c3F)c12 ; C#Cc1cccc2cc(O)cc(-c3ccc4c(N5CC6CCC(C5)N6)nc(OCC5(CN6CCC7(CCN(C)C7=O)C6)CC5)nc4c3F)c12 ; C#Cc1cccc2cc(O)cc(-c3ccc4c(N5CC6CCC(C5)N6)nc(OCC5(CN6CCC7(CCC(F)(F)CC7)C6)CC5)nc4c3F)c12 ; C#Cc1cccc2cc(O)cc(-c3ccc4c(N5CC6CCC(C5)N6)nc(OCC5(CN6CC7CCC(O)C7C6)CC5)nc4c3F)c12 ; C#Cc1cccc2cc(O)cc(-c3ccc4c(N5CC6CCC(C5)N6)nc(OCC5(CN6CC7CC(F)(F)C7C6)CC5)nc4c3F)c12]

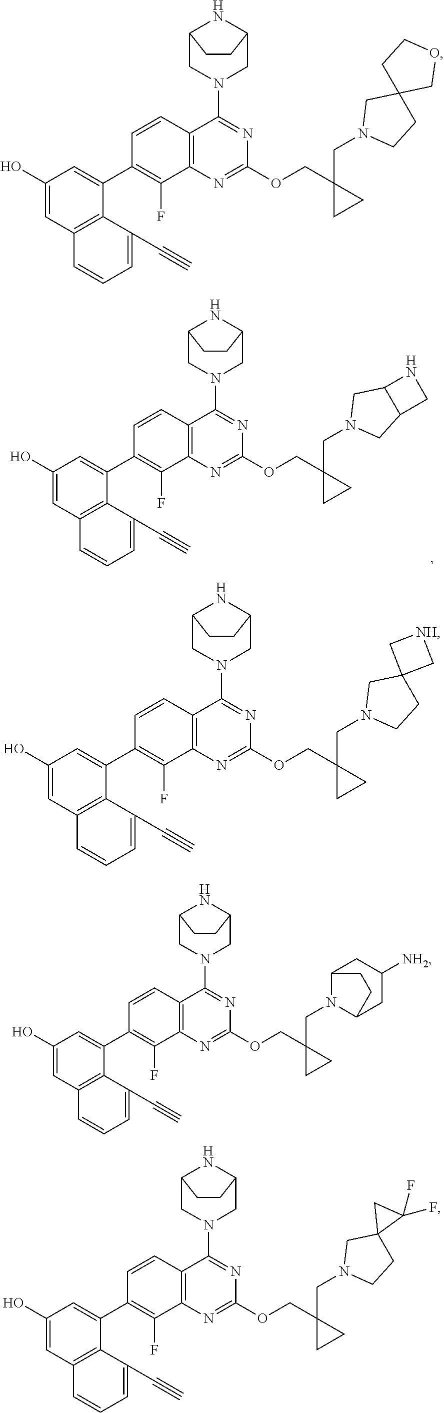 [SMILES: C#Cc1cccc2cc(O)cc(-c3ccc4c(N5CC6CCC(C5)N6)nc(OCC5(CN6CCC7(CCOC7)C6)CC5)nc4c3F)c12 ; C#Cc1cccc2cc(O)cc(-c3ccc4c(N5CC6CCC(C5)N6)nc(OCC5(CN6CC7CNC7C6)CC5)nc4c3F)c12 ; C#Cc1cccc2cc(O)cc(-c3ccc4c(N5CC6CCC(C5)N6)nc(OCC5(CN6CCC7(CNC7)C6)CC5)nc4c3F)c12 ; C#Cc1cccc2cc(O)cc(-c3ccc4c(N5CC6CCC(C5)N6)nc(OCC5(CN6C7CCC6CC(N)C7)CC5)nc4c3F)c12 ; C#Cc1cccc2cc(O)cc(-c3ccc4c(N5CC6CCC(C5)N6)nc(OCC5(CN6CCC7(C6)CC7(F)F)CC5)nc4c3F)c12]

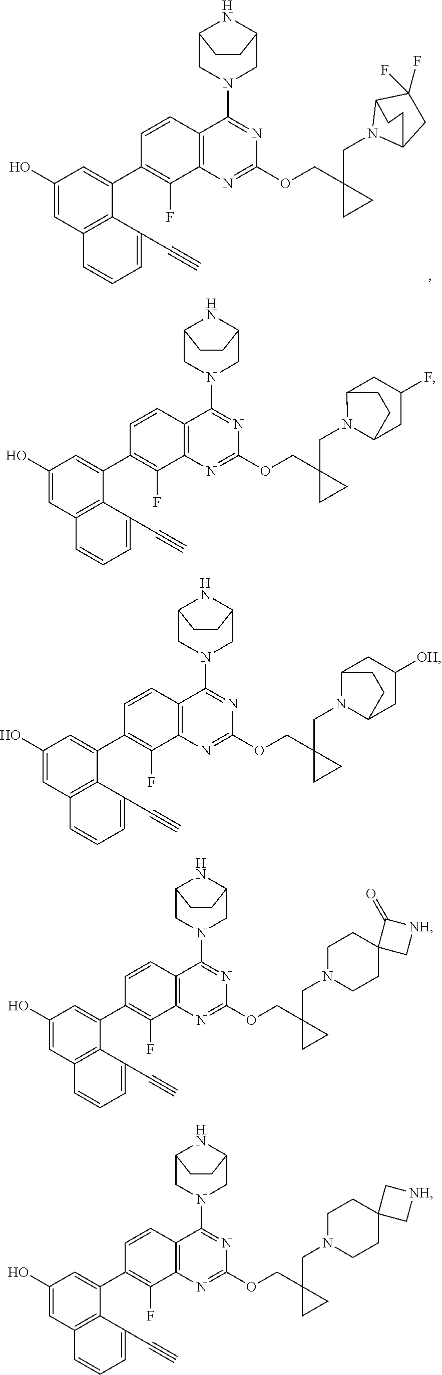 [SMILES: C#Cc1cccc2cc(O)cc(-c3ccc4c(N5CC6CCC(C5)N6)nc(OCC5(CN6C7CCC6C(F)(F)C7)CC5)nc4c3F)c12 ; C#Cc1cccc2cc(O)cc(-c3ccc4c(N5CC6CCC(C5)N6)nc(OCC5(CN6C7CCC6CC(F)C7)CC5)nc4c3F)c12 ; C#Cc1cccc2cc(O)cc(-c3ccc4c(N5CC6CCC(C5)N6)nc(OCC5(CN6C7CCC6CC(O)C7)CC5)nc4c3F)c12 ; C#Cc1cccc2cc(O)cc(-c3ccc4c(N5CC6CCC(C5)N6)nc(OCC5(CN6CCC7(CC6)CNC7=O)CC5)nc4c3F)c12 ; C#Cc1cccc2cc(O)cc(-c3ccc4c(N5CC6CCC(C5)N6)nc(OCC5(CN6CCC7(CC6)CNC7)CC5)nc4c3F)c12]

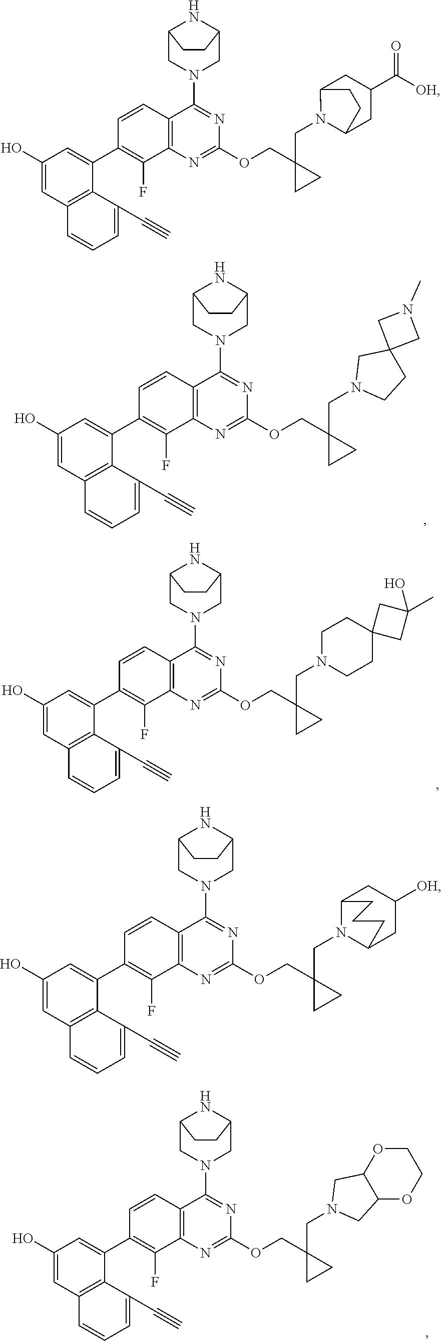 [SMILES: C#Cc1cccc2cc(O)cc(-c3ccc4c(N5CC6CCC(C5)N6)nc(OCC5(CN6C7CCC6CC(C(=O)O)C7)CC5)nc4c3F)c12 ; C#Cc1cccc2cc(O)cc(-c3ccc4c(N5CC6CCC(C5)N6)nc(OCC5(CN6CCC7(CN(C)C7)C6)CC5)nc4c3F)c12 ; C#Cc1cccc2cc(O)cc(-c3ccc4c(N5CC6CCC(C5)N6)nc(OCC5(CN6CCC7(CC6)CC(C)(O)C7)CC5)nc4c3F)c12 ; C#Cc1cccc2cc(O)cc(-c3ccc4c(N5CC6CCC(C5)N6)nc(OCC5(CN6C7CCCC6CC(O)C7)CC5)nc4c3F)c12 ; C#Cc1cccc2cc(O)cc(-c3ccc4c(N5CC6CCC(C5)N6)nc(OCC5(CN6CC7OCCOC7C6)CC5)nc4c3F)c12]

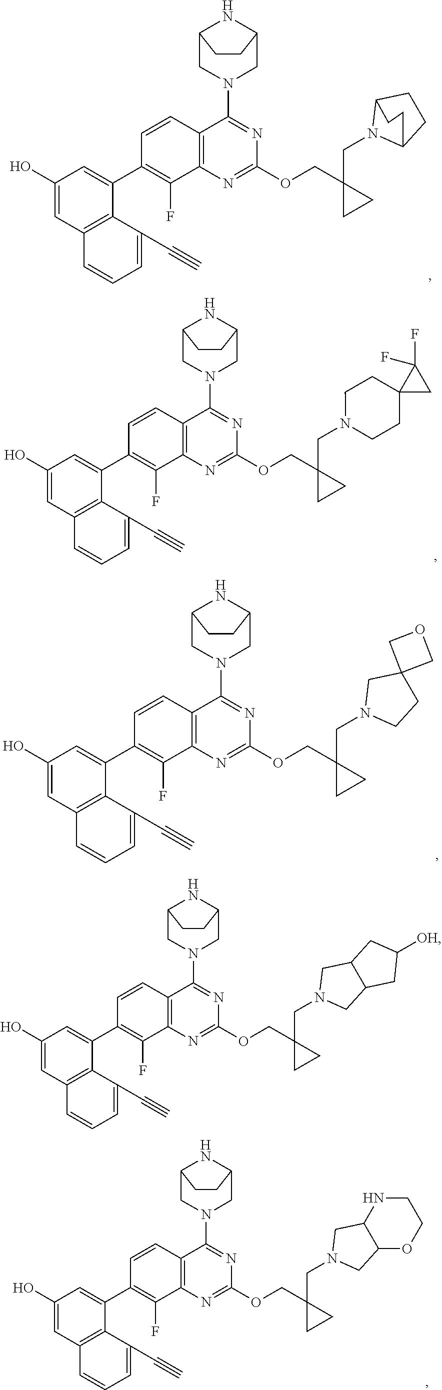 [SMILES: C#Cc1cccc2cc(O)cc(-c3ccc4c(N5CC6CCC(C5)N6)nc(OCC5(CN6C7CCC6CC7)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(COC7)C6)CC5)nc4c3F)c12 ; C#Cc1cccc2cc(O)cc(-c3ccc4c(N5CC6CCC(C5)N6)nc(OCC5(CN6CC7CC(O)CC7C6)CC5)nc4c3F)c12 ; C#Cc1cccc2cc(O)cc(-c3ccc4c(N5CC6CCC(C5)N6)nc(OCC5(CN6CC7NCCOC7C6)CC5)nc4c3F)c12]

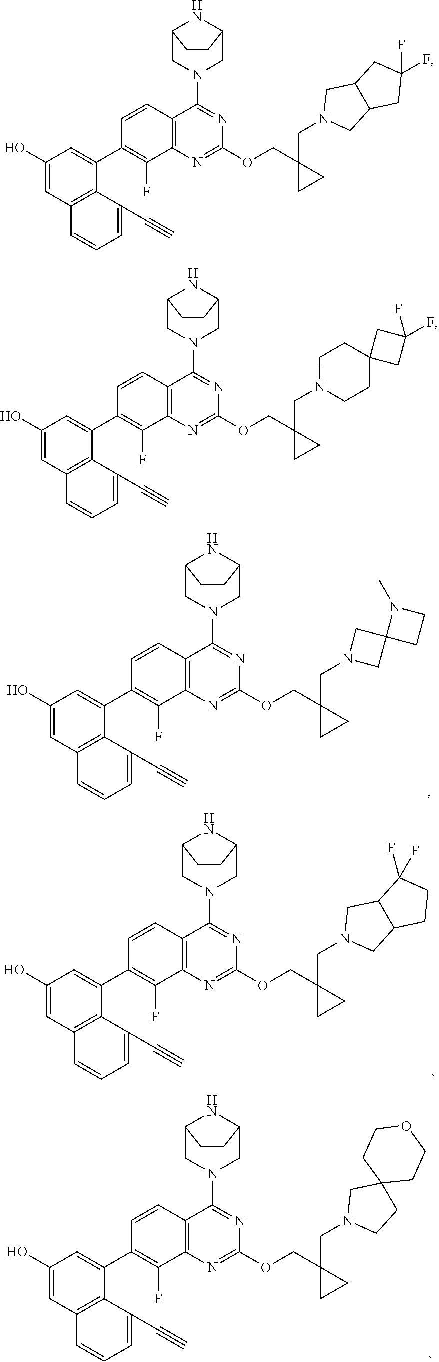 [SMILES: C#Cc1cccc2cc(O)cc(-c3ccc4c(N5CC6CCC(C5)N6)nc(OCC5(CN6CC7CC(F)(F)CC7C6)CC5)nc4c3F)c12 ; C#Cc1cccc2cc(O)cc(-c3ccc4c(N5CC6CCC(C5)N6)nc(OCC5(CN6CCC7(CC6)CC(F)(F)C7)CC5)nc4c3F)c12 ; C#Cc1cccc2cc(O)cc(-c3ccc4c(N5CC6CCC(C5)N6)nc(OCC5(CN6CC7(CCN7C)C6)CC5)nc4c3F)c12 ; C#Cc1cccc2cc(O)cc(-c3ccc4c(N5CC6CCC(C5)N6)nc(OCC5(CN6CC7CCC(F)(F)C7C6)CC5)nc4c3F)c12 ; C#Cc1cccc2cc(O)cc(-c3ccc4c(N5CC6CCC(C5)N6)nc(OCC5(CN6CCC7(CCOCC7)C6)CC5)nc4c3F)c12]

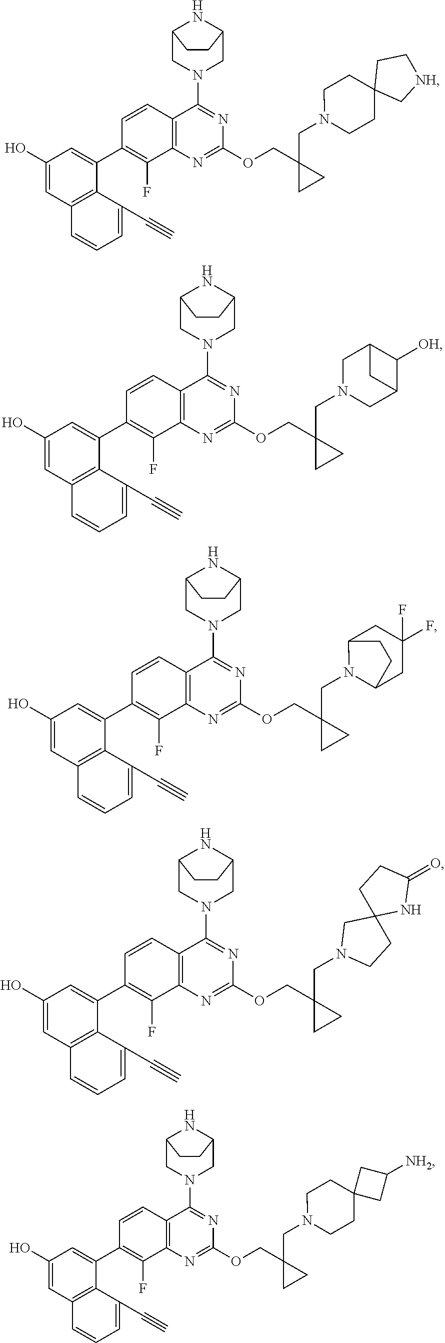 [SMILES: C#Cc1cccc2cc(O)cc(-c3ccc4c(N5CC6CCC(C5)N6)nc(OCC5(CN6CCC7(CCNC7)CC6)CC5)nc4c3F)c12 ; C#Cc1cccc2cc(O)cc(-c3ccc4c(N5CC6CCC(C5)N6)nc(OCC5(CN6CC7CC(C6)C7O)CC5)nc4c3F)c12 ; C#Cc1cccc2cc(O)cc(-c3ccc4c(N5CC6CCC(C5)N6)nc(OCC5(CN6C7CCC6CC(F)(F)C7)CC5)nc4c3F)c12 ; C#Cc1cccc2cc(O)cc(-c3ccc4c(N5CC6CCC(C5)N6)nc(OCC5(CN6CCC7(CCC(=O)N7)C6)CC5)nc4c3F)c12 ; C#Cc1cccc2cc(O)cc(-c3ccc4c(N5CC6CCC(C5)N6)nc(OCC5(CN6CCC7(CC6)CC(N)C7)CC5)nc4c3F)c12]

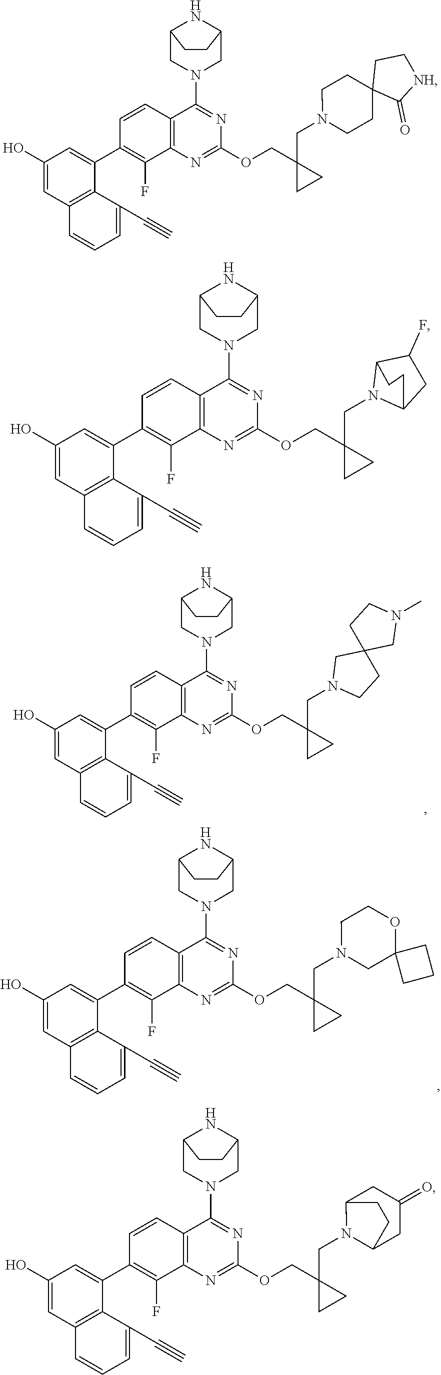 [SMILES: C#Cc1cccc2cc(O)cc(-c3ccc4c(N5CC6CCC(C5)N6)nc(OCC5(CN6CCC7(CCNC7=O)CC6)CC5)nc4c3F)c12 ; C#Cc1cccc2cc(O)cc(-c3ccc4c(N5CC6CCC(C5)N6)nc(OCC5(CN6C7CCC6C(F)C7)CC5)nc4c3F)c12 ; C#Cc1cccc2cc(O)cc(-c3ccc4c(N5CC6CCC(C5)N6)nc(OCC5(CN6CCC7(CCN(C)C7)C6)CC5)nc4c3F)c12 ; C#Cc1cccc2cc(O)cc(-c3ccc4c(N5CC6CCC(C5)N6)nc(OCC5(CN6CCOC7(CCC7)C6)CC5)nc4c3F)c12 ; C#Cc1cccc2cc(O)cc(-c3ccc4c(N5CC6CCC(C5)N6)nc(OCC5(CN6C7CCC6CC(=O)C7)CC5)nc4c3F)c12]

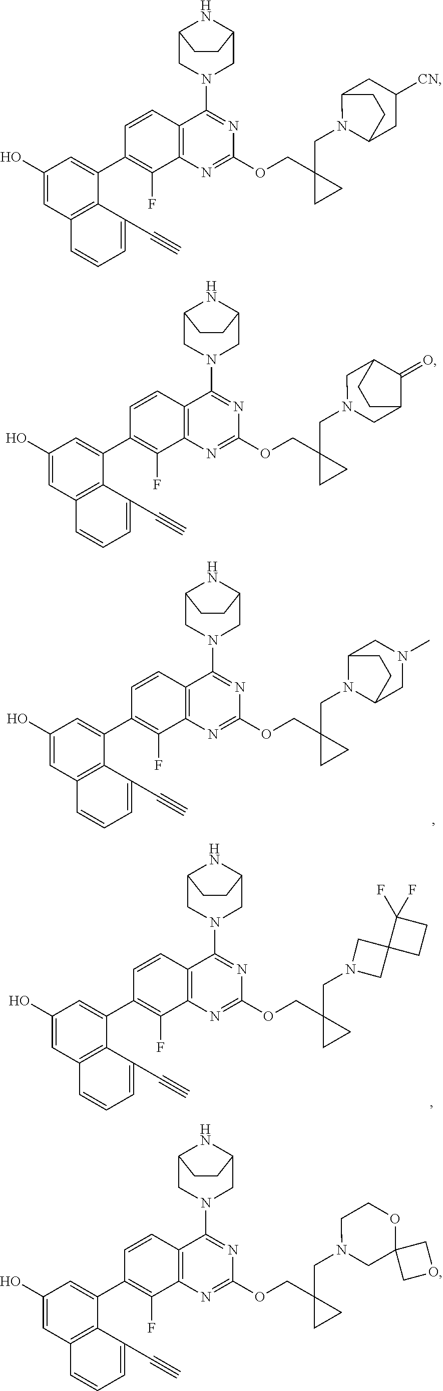 [SMILES: C#Cc1cccc2cc(O)cc(-c3ccc4c(N5CC6CCC(C5)N6)nc(OCC5(CN6C7CCC6CC(C#N)C7)CC5)nc4c3F)c12 ; C#Cc1cccc2cc(O)cc(-c3ccc4c(N5CC6CCC(C5)N6)nc(OCC5(CN6CC7CCC(C6)C7=O)CC5)nc4c3F)c12 ; C#Cc1cccc2cc(O)cc(-c3ccc4c(N5CC6CCC(C5)N6)nc(OCC5(CN6C7CCC6CN(C)C7)CC5)nc4c3F)c12 ; C#Cc1cccc2cc(O)cc(-c3ccc4c(N5CC6CCC(C5)N6)nc(OCC5(CN6CC7(CCC7(F)F)C6)CC5)nc4c3F)c12 ; C#Cc1cccc2cc(O)cc(-c3ccc4c(N5CC6CCC(C5)N6)nc(OCC5(CN6CCOC7(COC7)C6)CC5)nc4c3F)c12]

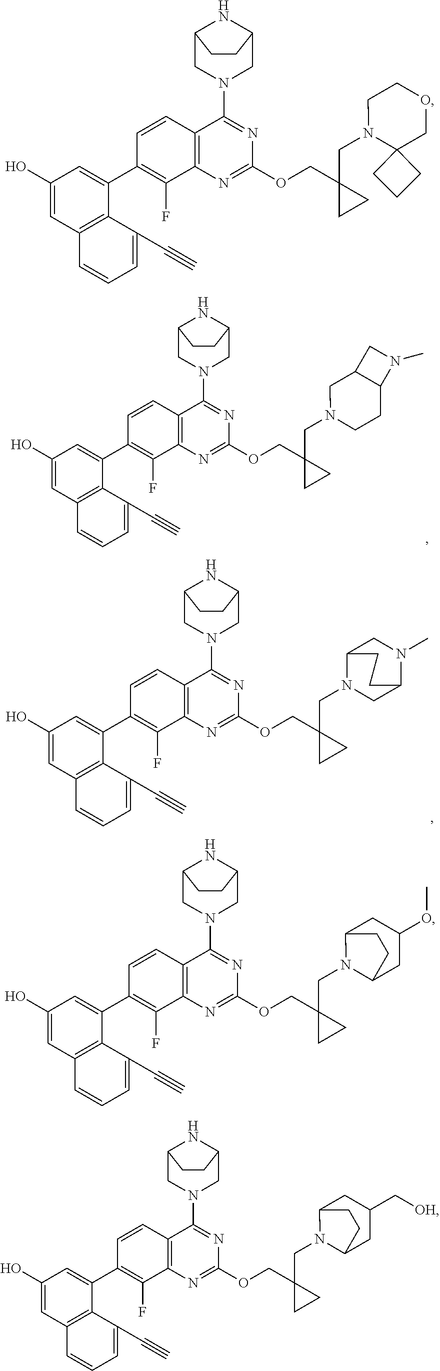 [SMILES: C#Cc1cccc2cc(O)cc(-c3ccc4c(N5CC6CCC(C5)N6)nc(OCC5(CN6CCOCC67CCC7)CC5)nc4c3F)c12 ; C#Cc1cccc2cc(O)cc(-c3ccc4c(N5CC6CCC(C5)N6)nc(OCC5(CN6CCC7C(C6)CN7C)CC5)nc4c3F)c12 ; C#Cc1cccc2cc(O)cc(-c3ccc4c(N5CC6CCC(C5)N6)nc(OCC5(CN6CC7CCC6CN7C)CC5)nc4c3F)c12 ; C#Cc1cccc2cc(O)cc(-c3ccc4c(N5CC6CCC(C5)N6)nc(OCC5(CN6C7CCC6CC(OC)C7)CC5)nc4c3F)c12 ; C#Cc1cccc2cc(O)cc(-c3ccc4c(N5CC6CCC(C5)N6)nc(OCC5(CN6C7CCC6CC(CO)C7)CC5)nc4c3F)c12]

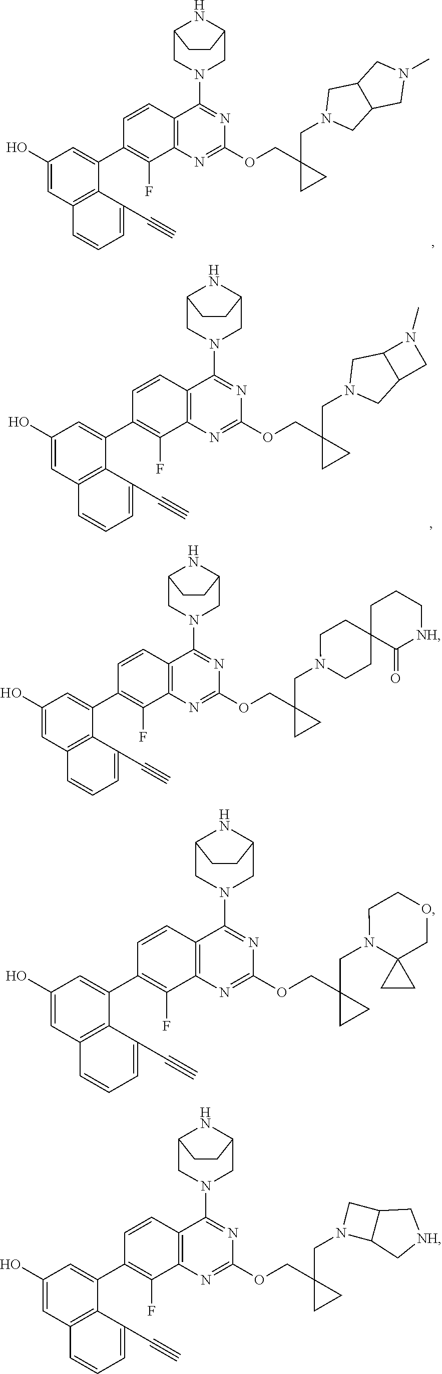 [SMILES: C#Cc1cccc2cc(O)cc(-c3ccc4c(N5CC6CCC(C5)N6)nc(OCC5(CN6CC7CN(C)CC7C6)CC5)nc4c3F)c12 ; C#Cc1cccc2cc(O)cc(-c3ccc4c(N5CC6CCC(C5)N6)nc(OCC5(CN6CC7CN(C)C7C6)CC5)nc4c3F)c12 ; C#Cc1cccc2cc(O)cc(-c3ccc4c(N5CC6CCC(C5)N6)nc(OCC5(CN6CCC7(CCCNC7=O)CC6)CC5)nc4c3F)c12 ; C#Cc1cccc2cc(O)cc(-c3ccc4c(N5CC6CCC(C5)N6)nc(OCC5(CN6CCOCC67CC7)CC5)nc4c3F)c12 ; C#Cc1cccc2cc(O)cc(-c3ccc4c(N5CC6CCC(C5)N6)nc(OCC5(CN6CC7CNCC76)CC5)nc4c3F)c12]

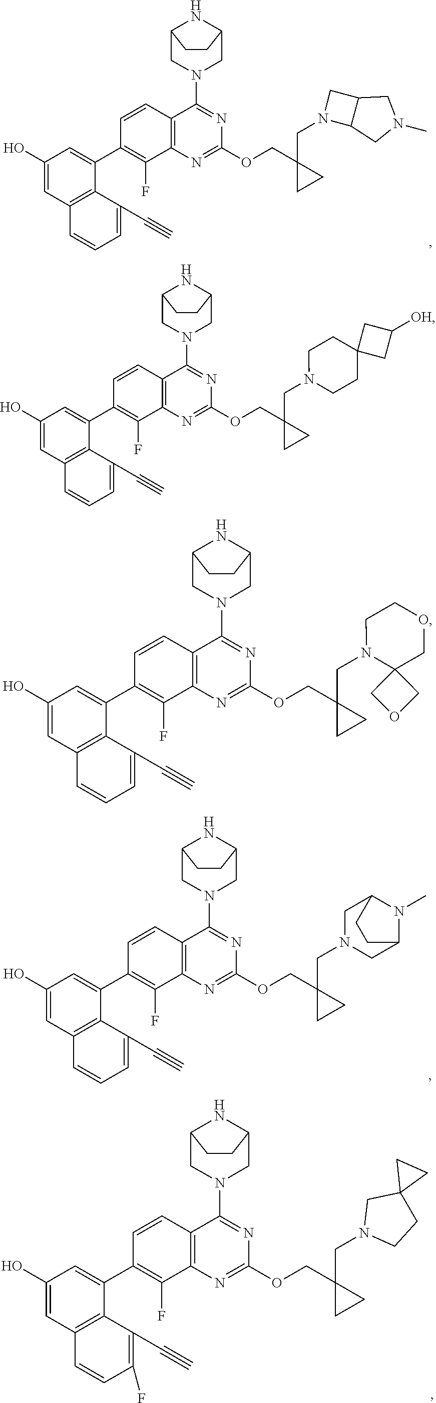 [SMILES: C#Cc1cccc2cc(O)cc(-c3ccc4c(N5CC6CCC(C5)N6)nc(OCC5(CN6CC7CN(C)CC76)CC5)nc4c3F)c12 ; C#Cc1cccc2cc(O)cc(-c3ccc4c(N5CC6CCC(C5)N6)nc(OCC5(CN6CCC7(CC6)CC(O)C7)CC5)nc4c3F)c12 ; C#Cc1cccc2cc(O)cc(-c3ccc4c(N5CC6CCC(C5)N6)nc(OCC5(CN6CCOCC67COC7)CC5)nc4c3F)c12 ; C#Cc1cccc2cc(O)cc(-c3ccc4c(N5CC6CCC(C5)N6)nc(OCC5(CN6CC7CCC(C6)N7C)CC5)nc4c3F)c12 ; C#Cc1c(F)ccc2cc(O)cc(-c3ccc4c(N5CC6CCC(C5)N6)nc(OCC5(CN6CCC7(CC7)C6)CC5)nc4c3F)c12]

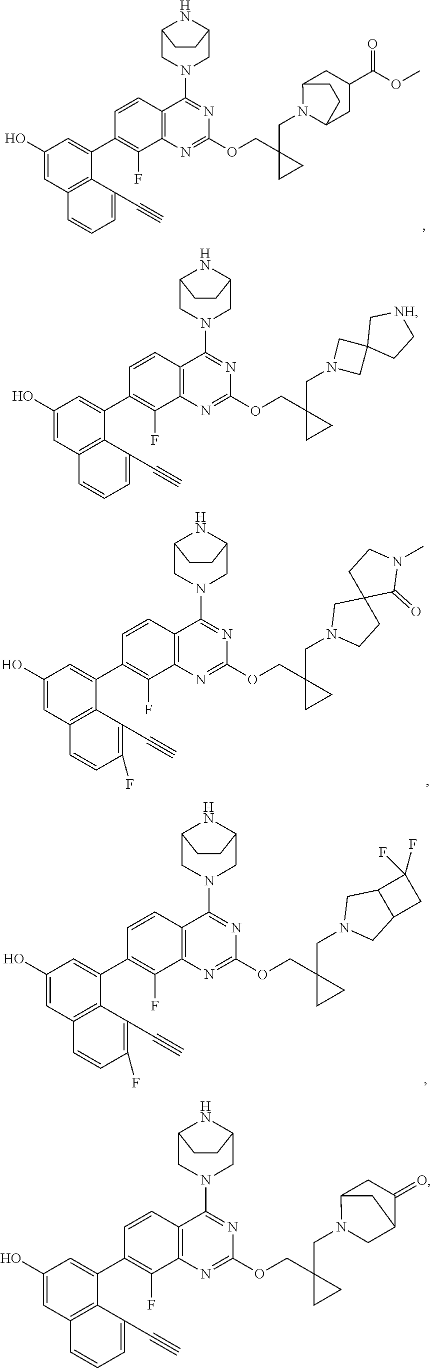 [SMILES: C#Cc1cccc2cc(O)cc(-c3ccc4c(N5CC6CCC(C5)N6)nc(OCC5(CN6C7CCC6CC(C(=O)OC)C7)CC5)nc4c3F)c12 ; C#Cc1cccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CCC7(CCN(C)C7=O)C6)CC5)nc4c3F)c12 ; C#Cc1c(F)ccc2cc(O)cc(-c3ccc4c(N5CC6CCC(C5)N6)nc(OCC5(CN6CC7CC(F)(F)C7C6)CC5)nc4c3F)c12 ; C#Cc1cccc2cc(O)cc(-c3ccc4c(N5CC6CCC(C5)N6)nc(OCC5(CN6CC7CC6CC7=O)CC5)nc4c3F)c12]

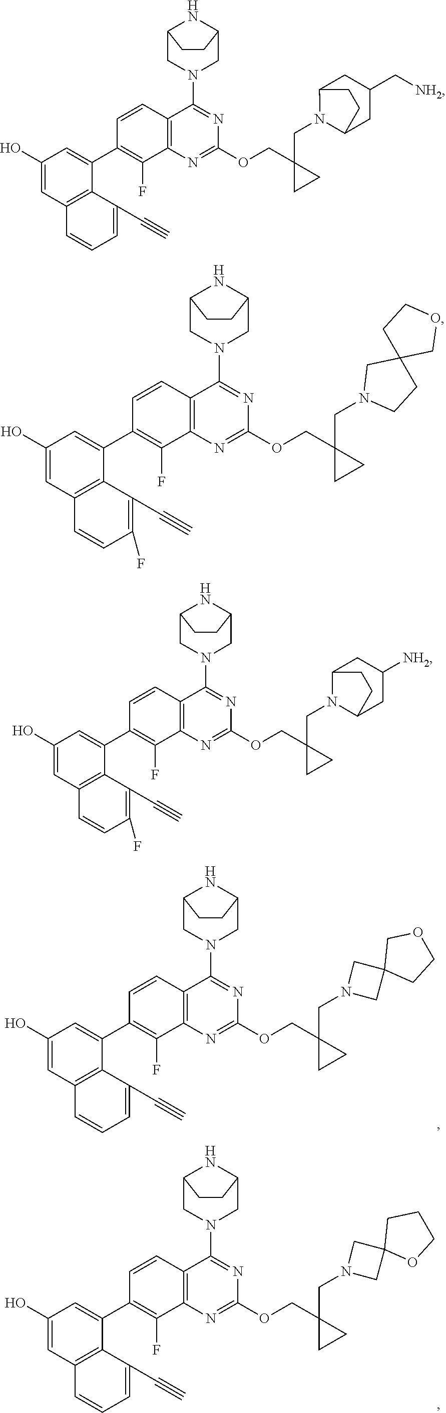 [SMILES: C#Cc1cccc2cc(O)cc(-c3ccc4c(N5CC6CCC(C5)N6)nc(OCC5(CN6C7CCC6CC(CN)C7)CC5)nc4c3F)c12 ; C#Cc1c(F)ccc2cc(O)cc(-c3ccc4c(N5CC6CCC(C5)N6)nc(OCC5(CN6CCC7(CCOC7)C6)CC5)nc4c3F)c12 ; C#Cc1c(F)ccc2cc(O)cc(-c3ccc4c(N5CC6CCC(C5)N6)nc(OCC5(CN6C7CCC6CC(N)C7)CC5)nc4c3F)c12 ; C#Cc1cccc2cc(O)cc(-c3ccc4c(N5CC6CCC(C5)N6)nc(OCC5(CN6CC7(CCOC7)C6)CC5)nc4c3F)c12 ; C#Cc1cccc2cc(O)cc(-c3ccc4c(N5CC6CCC(C5)N6)nc(OCC5(CN6CC7(CCCO7)C6)CC5)nc4c3F)c12]

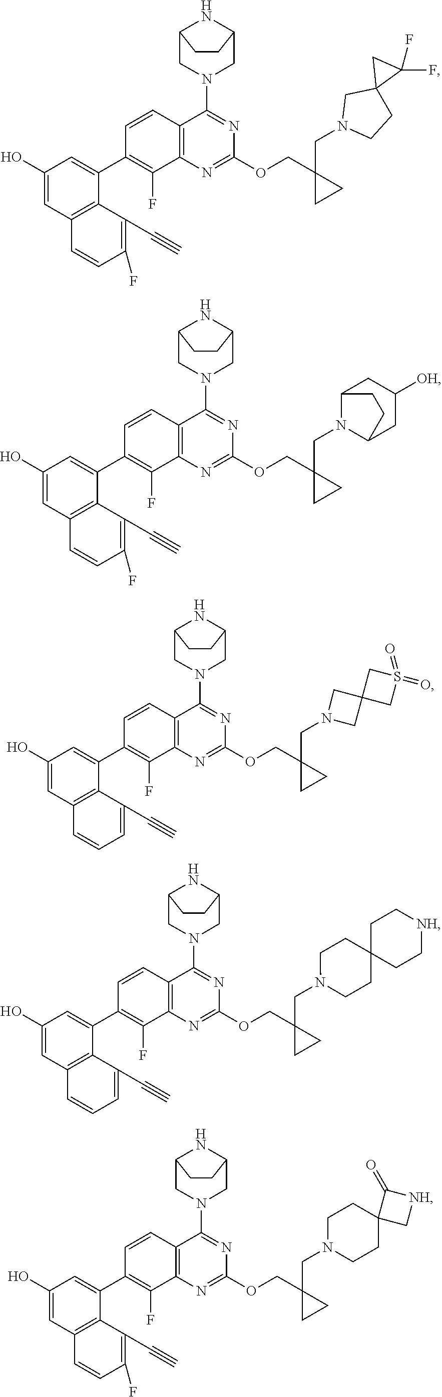 [SMILES: C#Cc1c(F)ccc2cc(O)cc(-c3ccc4c(N5CC6CCC(C5)N6)nc(OCC5(CN6CCC7(C6)CC7(F)F)CC5)nc4c3F)c12 ; C#Cc1c(F)ccc2cc(O)cc(-c3ccc4c(N5CC6CCC(C5)N6)nc(OCC5(CN6C7CCC6CC(O)C7)CC5)nc4c3F)c12 ; C#Cc1cccc2cc(O)cc(-c3ccc4c(N5CC6CCC(C5)N6)nc(OCC5(CN6CC7(C6)CS(=O)(=O)C7)CC5)nc4c3F)c12 ; C#Cc1cccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CCC7(CC6)CNC7=O)CC5)nc4c3F)c12]

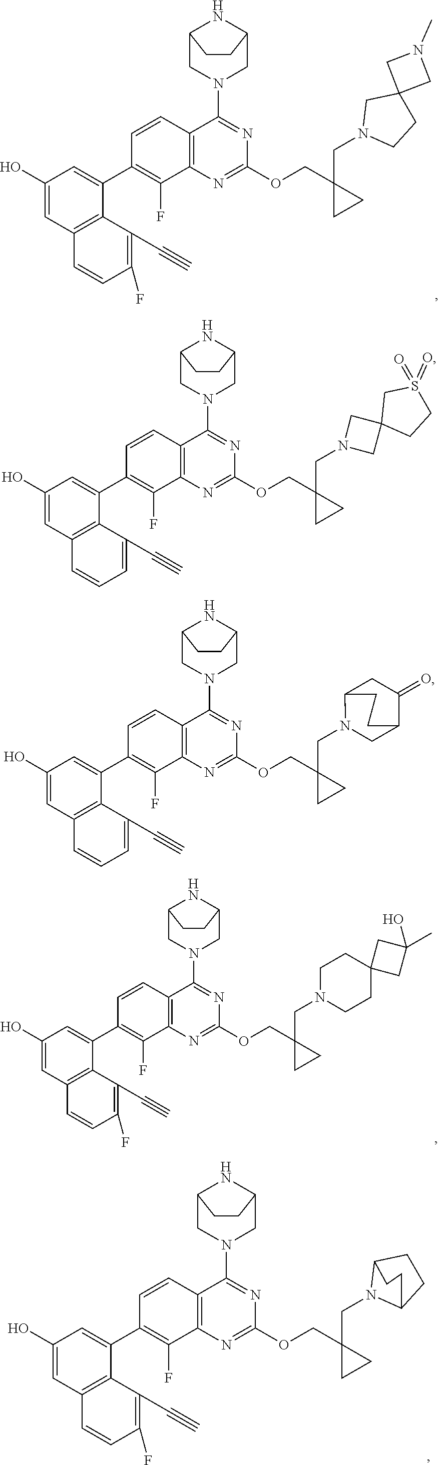 [SMILES: C#Cc1c(F)ccc2cc(O)cc(-c3ccc4c(N5CC6CCC(C5)N6)nc(OCC5(CN6CCC7(CN(C)C7)C6)CC5)nc4c3F)c12 ; C#Cc1cccc2cc(O)cc(-c3ccc4c(N5CC6CCC(C5)N6)nc(OCC5(CN6CC7(CCS(=O)(=O)C7)C6)CC5)nc4c3F)c12 ; C#Cc1cccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6CCC7(CC6)CC(C)(O)C7)CC5)nc4c3F)c12 ; C#Cc1c(F)ccc2cc(O)cc(-c3ccc4c(N5CC6CCC(C5)N6)nc(OCC5(CN6C7CCC6CC7)CC5)nc4c3F)c12]

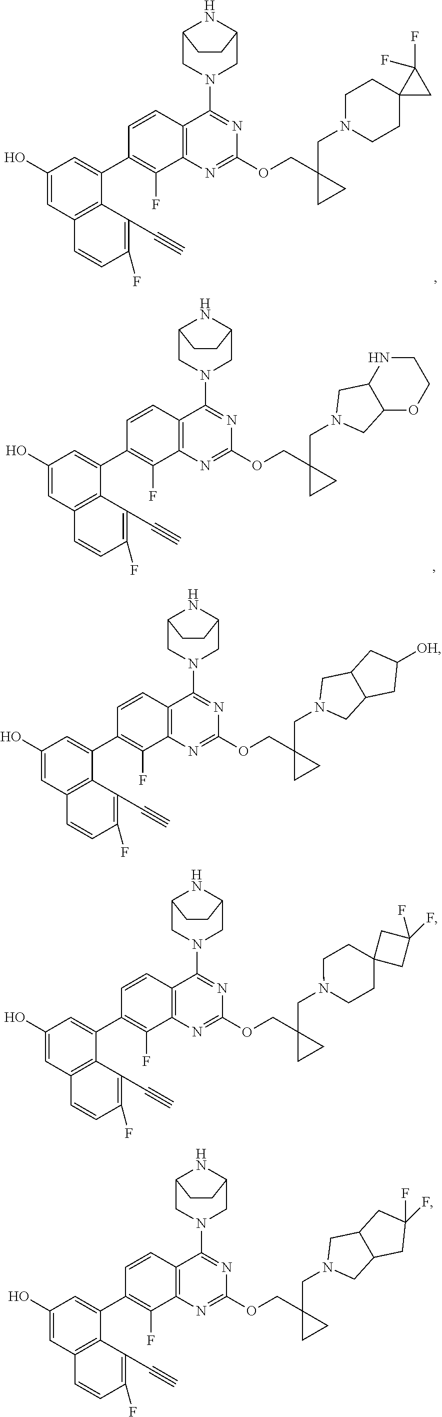 [SMILES: C#Cc1c(F)ccc2cc(O)cc(-c3ccc4c(N5CC6CCC(C5)N6)nc(OCC5(CN6CCC7(CC6)CC7(F)F)CC5)nc4c3F)c12 ; C#Cc1c(F)ccc2cc(O)cc(-c3ccc4c(N5CC6CCC(C5)N6)nc(OCC5(CN6CC7NCCOC7C6)CC5)nc4c3F)c12 ; C#Cc1c(F)ccc2cc(O)cc(-c3ccc4c(N5CC6CCC(C5)N6)nc(OCC5(CN6CC7CC(O)CC7C6)CC5)nc4c3F)c12 ; C#Cc1c(F)ccc2cc(O)cc(-c3ccc4c(N5CC6CCC(C5)N6)nc(OCC5(CN6CCC7(CC6)CC(F)(F)C7)CC5)nc4c3F)c12 ; C#Cc1c(F)ccc2cc(O)cc(-c3ccc4c(N5CC6CCC(C5)N6)nc(OCC5(CN6CC7CC(F)(F)CC7C6)CC5)nc4c3F)c12]

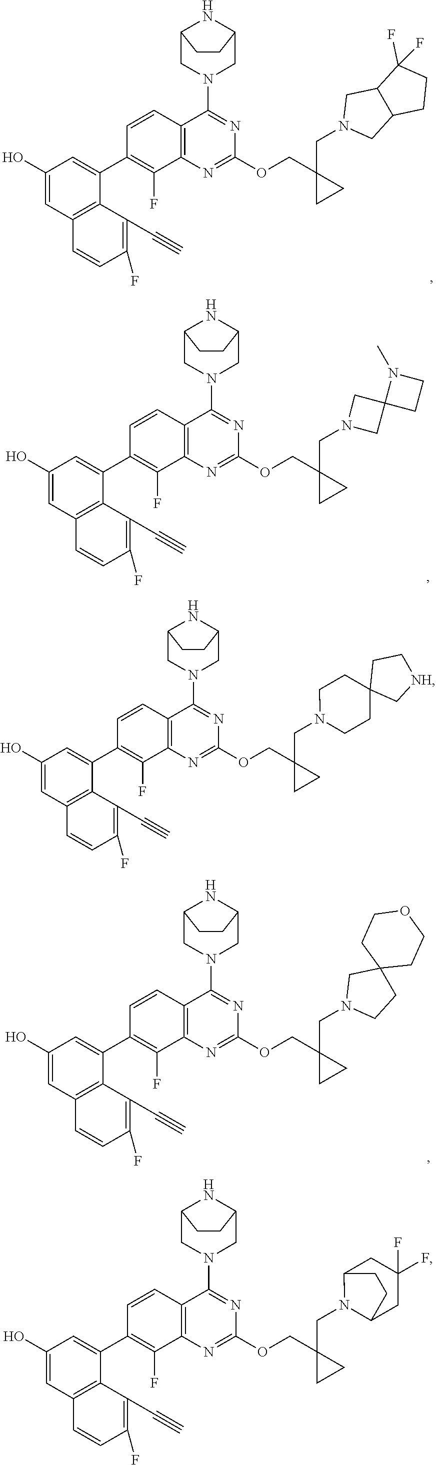 [SMILES: C#Cc1c(F)ccc2cc(O)cc(-c3ccc4c(N5CC6CCC(C5)N6)nc(OCC5(CN6CC7CCC(F)(F)C7C6)CC5)nc4c3F)c12 ; C#Cc1c(F)ccc2cc(O)cc(-c3ccc4c(N5CC6CCC(C5)N6)nc(OCC5(CN6CC7(CCN7C)C6)CC5)nc4c3F)c12 ; C#Cc1c(F)ccc2cc(O)cc(-c3ccc4c(N5CC6CCC(C5)N6)nc(OCC5(CN6CCC7(CCNC7)CC6)CC5)nc4c3F)c12 ; C#Cc1c(F)ccc2cc(O)cc(-c3ccc4c(N5CC6CCC(C5)N6)nc(OCC5(CN6CCC7(CCOCC7)C6)CC5)nc4c3F)c12 ; C#Cc1c(F)ccc2cc(O)cc(-c3ccc4c(N5CC6CCC(C5)N6)nc(OCC5(CN6C7CCC6CC(F)(F)C7)CC5)nc4c3F)c12]

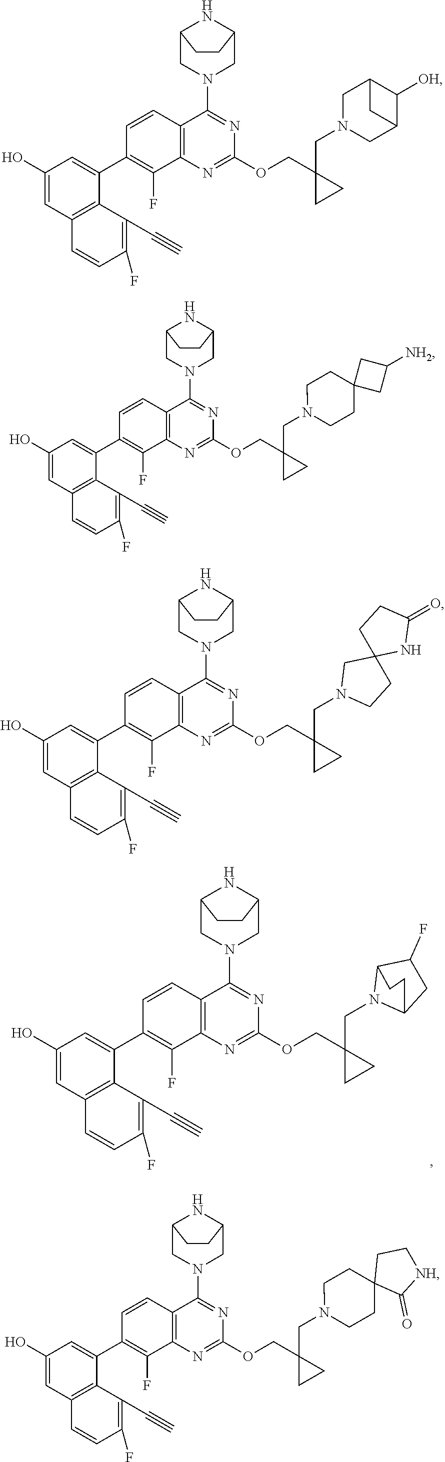 [SMILES: C#Cc1c(F)ccc2cc(O)cc(-c3ccc4c(N5CC6CCC(C5)N6)nc(OCC5(CN6CC7CC(C6)C7O)CC5)nc4c3F)c12 ; C#Cc1c(F)ccc2cc(O)cc(-c3ccc4c(N5CC6CCC(C5)N6)nc(OCC5(CN6CCC7(CC6)CC(N)C7)CC5)nc4c3F)c12 ; C#Cc1c(F)ccc2cc(O)cc(-c3ccc4c(N5CC6CCC(C5)N6)nc(OCC5(CN6CCC7(CCC(=O)N7)C6)CC5)nc4c3F)c12 ; C#Cc1c(F)ccc2cc(O)cc(-c3ccc4c(N5CC6CCC(C5)N6)nc(OCC5(CN6C7CCC6C(F)C7)CC5)nc4c3F)c12 ; C#Cc1c(F)ccc2cc(O)cc(-c3ccc4c(N5CC6CCC(C5)N6)nc(OCC5(CN6CCC7(CCNC7=O)CC6)CC5)nc4c3F)c12]

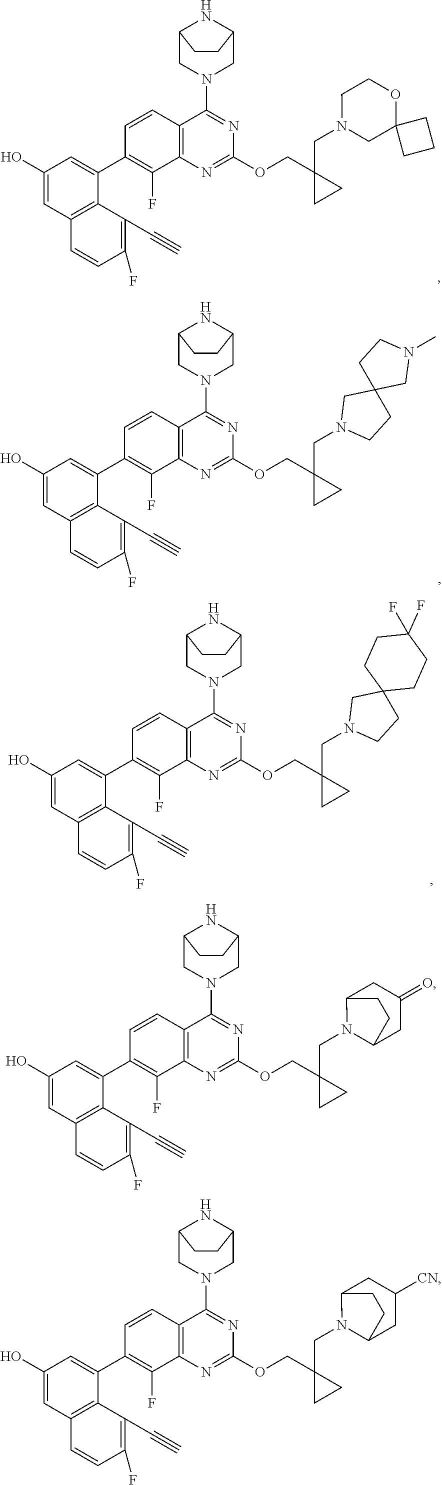 [SMILES: C#Cc1c(F)ccc2cc(O)cc(-c3ccc4c(N5CC6CCC(C5)N6)nc(OCC5(CN6CCOC7(CCC7)C6)CC5)nc4c3F)c12 ; C#Cc1c(F)ccc2cc(O)cc(-c3ccc4c(N5CC6CCC(C5)N6)nc(OCC5(CN6CCC7(CCN(C)C7)C6)CC5)nc4c3F)c12 ; C#Cc1c(F)ccc2cc(O)cc(-c3ccc4c(N5CC6CCC(C5)N6)nc(OCC5(CN6CCC7(CCC(F)(F)CC7)C6)CC5)nc4c3F)c12 ; C#Cc1c(F)ccc2cc(O)cc(-c3ccc4c(N5CC6CCC(C5)N6)nc(OCC5(CN6C7CCC6CC(=O)C7)CC5)nc4c3F)c12 ; C#Cc1c(F)ccc2cc(O)cc(-c3ccc4c(N5CC6CCC(C5)N6)nc(OCC5(CN6C7CCC6CC(C#N)C7)CC5)nc4c3F)c12]

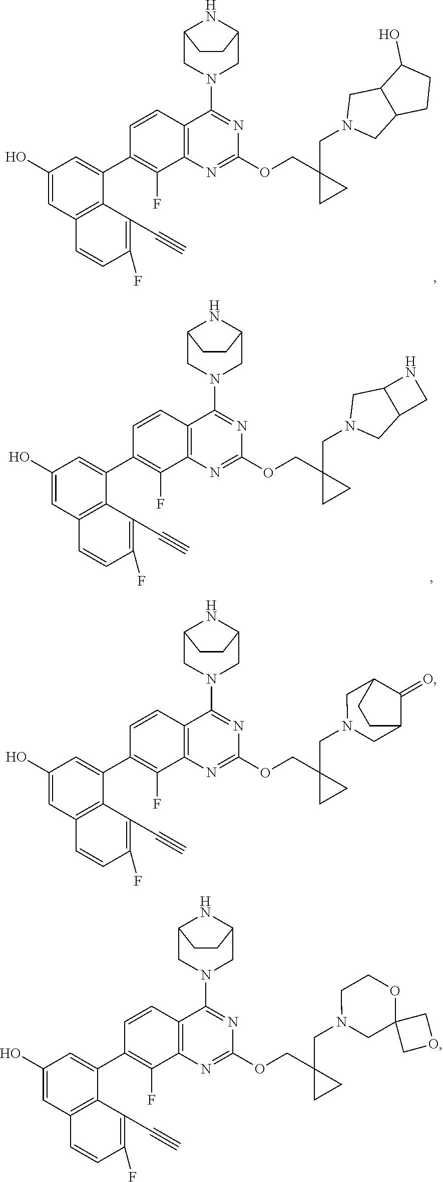 [SMILES: C#Cc1c(F)ccc2cc(O)cc(-c3ccc4c(N5CC6CCC(C5)N6)nc(OCC5(CN6CC7CCC(O)C7C6)CC5)nc4c3F)c12 ; C#Cc1c(F)ccc2cc(O)cc(-c3ccc4c(N5CC6CCC(C5)N6)nc(OCC5(CN6CC7CNC7C6)CC5)nc4c3F)c12 ; C#Cc1c(F)ccc2cc(O)cc(-c3ccc4c(N5CC6CCC(C5)N6)nc(OCC5(CN6CC7CCC(C6)C7=O)CC5)nc4c3F)c12 ; C#Cc1c(F)ccc2cc(O)cc(-c3ccc4c(N5CC6CCC(C5)N6)nc(OCC5(CN6CCOC7(COC7)C6)CC5)nc4c3F)c12]

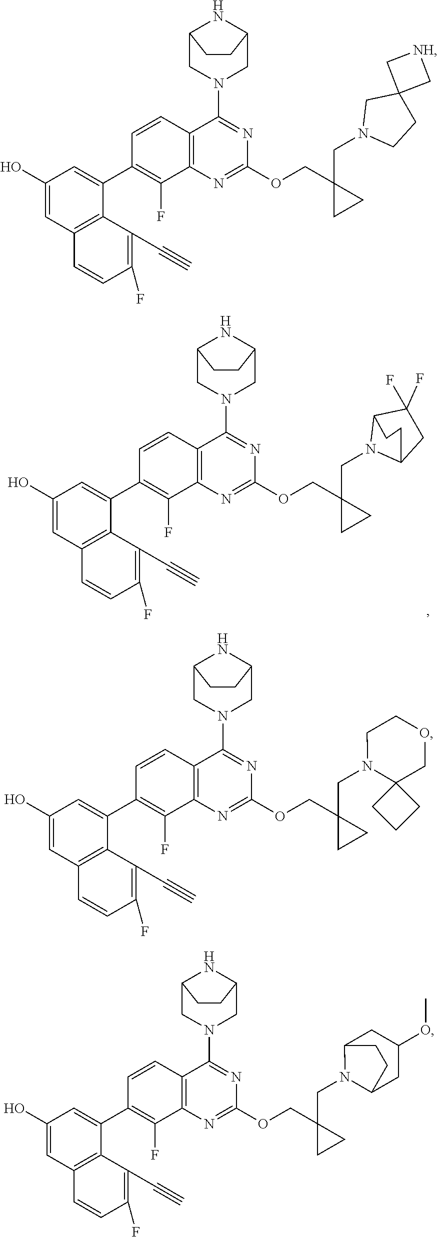 [SMILES: C#Cc1c(F)ccc2cc(O)cc(-c3ccc4c(N5CC6CCC(C5)N6)nc(OCC5(CN6CCC7(CNC7)C6)CC5)nc4c3F)c12 ; C#Cc1c(F)ccc2cc(O)cc(-c3ccc4c(N5CC6CCC(C5)N6)nc(OCC5(CN6C7CCC6C(F)(F)C7)CC5)nc4c3F)c12 ; C#Cc1c(F)ccc2cc(O)cc(-c3ccc4c(N5CC6CCC(C5)N6)nc(OCC5(CN6CCOCC67CCC7)CC5)nc4c3F)c12 ; C#Cc1c(F)ccc2cc(O)cc(-c3ccc4c(N5CC6CCC(C5)N6)nc(OCC5(CN6C7CCC6CC(OC)C7)CC5)nc4c3F)c12]

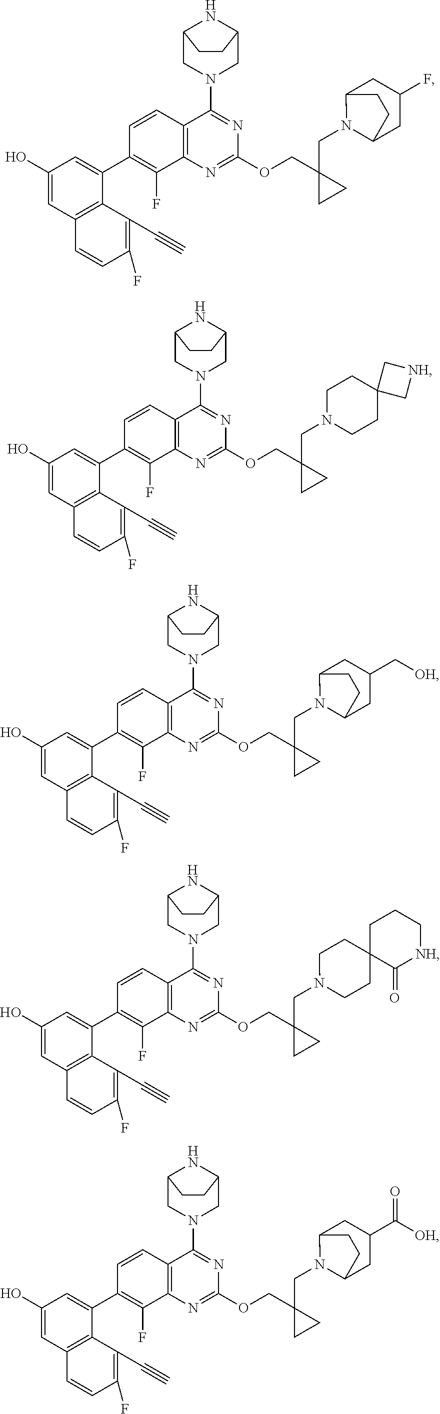 [SMILES: C#Cc1c(F)ccc2cc(O)cc(-c3ccc4c(N5CC6CCC(C5)N6)nc(OCC5(CN6C7CCC6CC(F)C7)CC5)nc4c3F)c12 ; C#Cc1c(F)ccc2cc(O)cc(-c3ccc4c(N5CC6CCC(C5)N6)nc(OCC5(CN6CCC7(CC6)CNC7)CC5)nc4c3F)c12 ; C#Cc1c(F)ccc2cc(O)cc(-c3ccc4c(N5CC6CCC(C5)N6)nc(OCC5(CN6C7CCC6CC(CO)C7)CC5)nc4c3F)c12 ; C#Cc1c(F)ccc2cc(O)cc(-c3ccc4c(N5CC6CCC(C5)N6)nc(OCC5(CN6CCC7(CCCNC7=O)CC6)CC5)nc4c3F)c12 ; C#Cc1c(F)ccc2cc(O)cc(-c3ccc4c(N5CC6CCC(C5)N6)nc(OCC5(CN6C7CCC6CC(C(=O)O)C7)CC5)nc4c3F)c12]

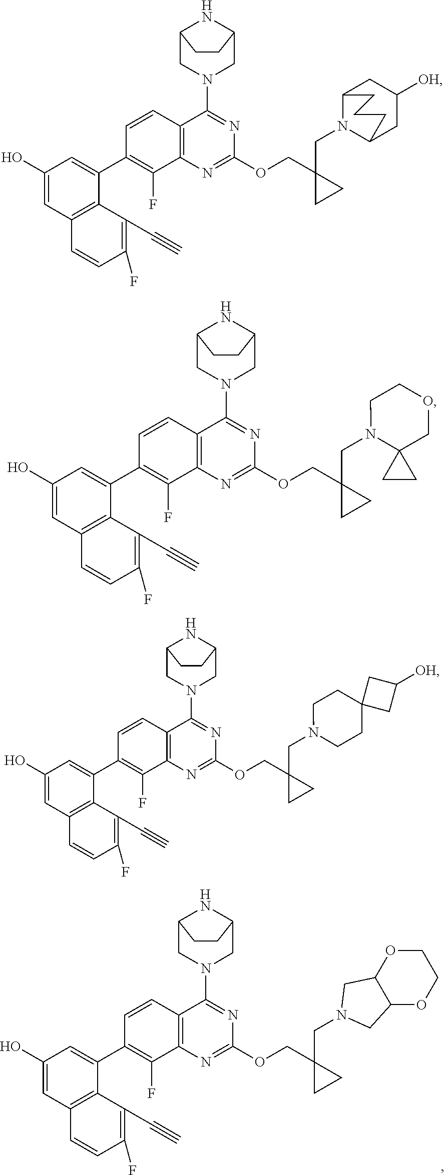 [SMILES: C#Cc1c(F)ccc2cc(O)cc(-c3ccc4c(N5CC6CCC(C5)N6)nc(OCC5(CN6C7CCCC6CC(O)C7)CC5)nc4c3F)c12 ; C#Cc1c(F)ccc2cc(O)cc(-c3ccc4c(N5CC6CCC(C5)N6)nc(OCC5(CN6CCOCC67CC7)CC5)nc4c3F)c12 ; C#Cc1c(F)ccc2cc(O)cc(-c3ccc4c(N5CC6CCC(C5)N6)nc(OCC5(CN6CCC7(CC6)CC(O)C7)CC5)nc4c3F)c12 ; C#Cc1c(F)ccc2cc(O)cc(-c3ccc4c(N5CC6CCC(C5)N6)nc(OCC5(CN6CC7OCCOC7C6)CC5)nc4c3F)c12]

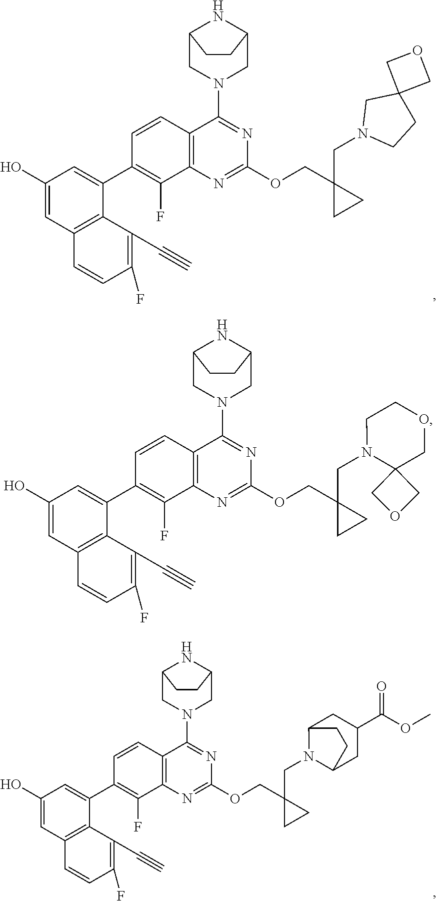 [SMILES: C#Cc1c(F)ccc2cc(O)cc(-c3ccc4c(N5CC6CCC(C5)N6)nc(OCC5(CN6CCC7(COC7)C6)CC5)nc4c3F)c12 ; C#Cc1c(F)ccc2cc(O)cc(-c3ccc4c(N5CC6CCC(C5)N6)nc(OCC5(CN6CCOCC67COC7)CC5)nc4c3F)c12 ; C#Cc1c(F)ccc2cc(O)cc(-c3ccc4c(N5CC6CCC(C5)N6)nc(OCC5(CN6C7CCC6CC(C(=O)OC)C7)CC5)nc4c3F)c12]

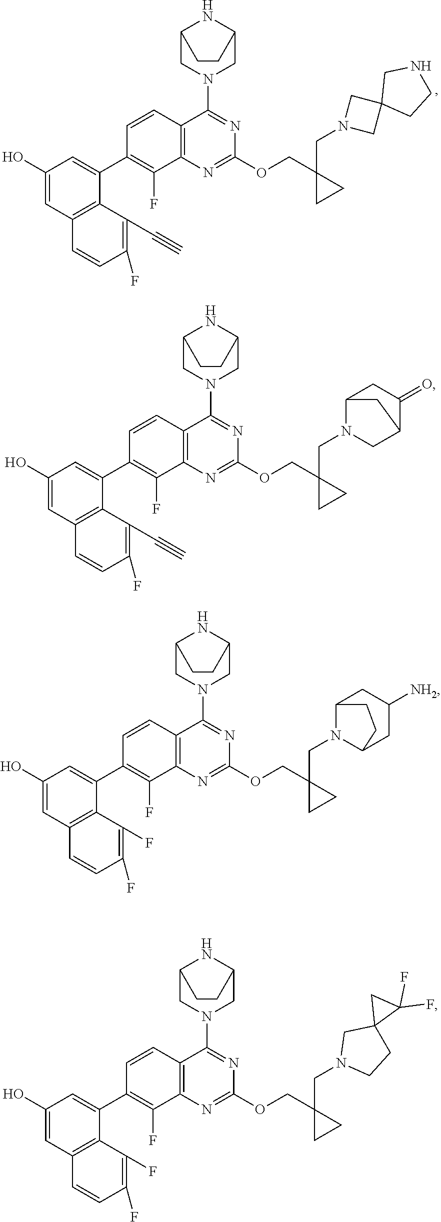 [SMILES: C#Cc1c(F)ccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CC7CC6CC7=O)CC5)nc4c3F)c12 ; NC1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CCC6(C5)CC6(F)F)CC4)nc3c2F)c2c(F)c(F)ccc2c1]

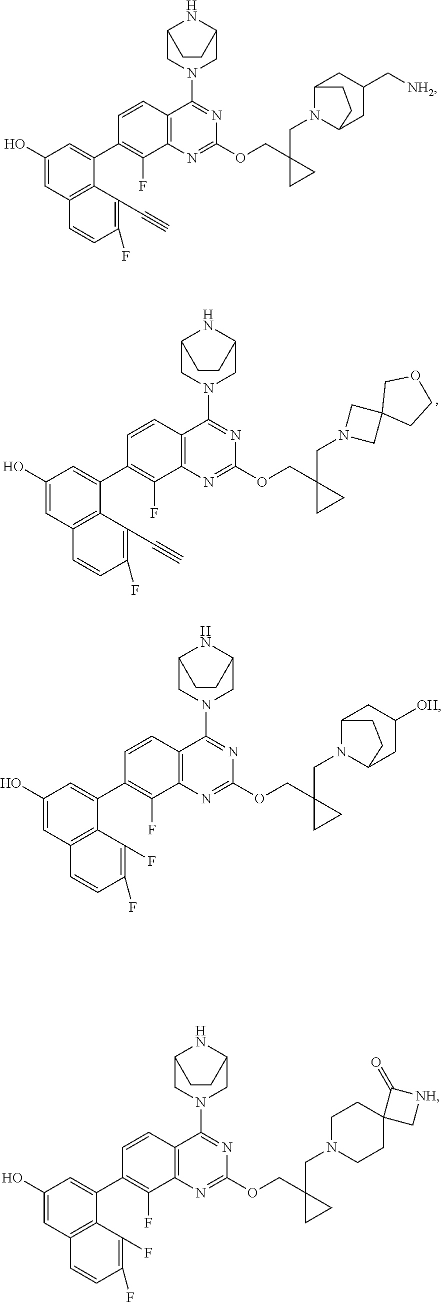 [SMILES: C#Cc1c(F)ccc2cc(O)cc(-c3ccc4c(N5CC6CCC(C5)N6)nc(OCC5(CN6C7CCC6CC(CN)C7)CC5)nc4c3F)c12 ; C#Cc1c(F)ccc2cc(O)cc(-c3ccc4c(N5CC6CCC(C5)N6)nc(OCC5(CN6CC7(CCOC7)C6)CC5)nc4c3F)c12 ; Oc1cc(-c2ccc3c(N4CC5CCC(C4)N5)nc(OCC4(CN5C6CCC5CC(O)C6)CC4)nc3c2F)c2c(F)c(F)ccc2c1 ; O=C1NCC12CCN(CC1(COc3nc(N4CC5CCC(C4)N5)c4ccc(-c5cc(O)cc6ccc(F)c(F)c56)c(F)c4n3)CC1)CC2]

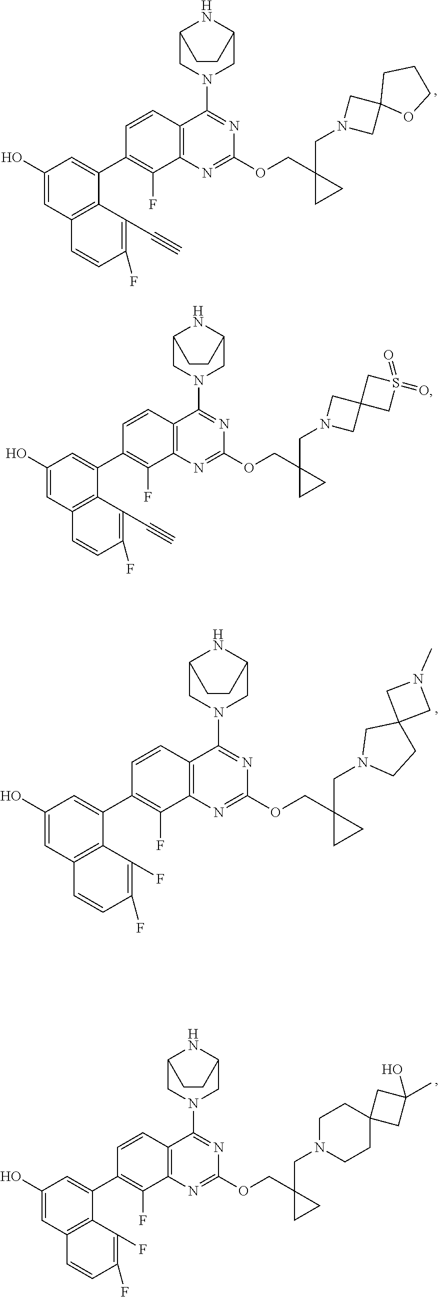 [SMILES: C#Cc1c(F)ccc2cc(O)cc(-c3ccc4c(N5CC6CCC(C5)N6)nc(OCC5(CN6CC7(CCCO7)C6)CC5)nc4c3F)c12 ; C#Cc1c(F)ccc2cc(O)cc(-c3ccc4c(N5CC6CCC(C5)N6)nc(OCC5(CN6CC7(C6)CS(=O)(=O)C7)CC5)nc4c3F)c12 ; CN1CC2(CCN(CC3(COc4nc(N5CC6CCC(C5)N6)c5ccc(-c6cc(O)cc7ccc(F)c(F)c67)c(F)c5n4)CC3)C2)C1 ; CC1(O)CC2(CCN(CC3(COc4nc(N5CC6CCC(C5)N6)c5ccc(-c6cc(O)cc7ccc(F)c(F)c67)c(F)c5n4)CC3)CC2)C1]

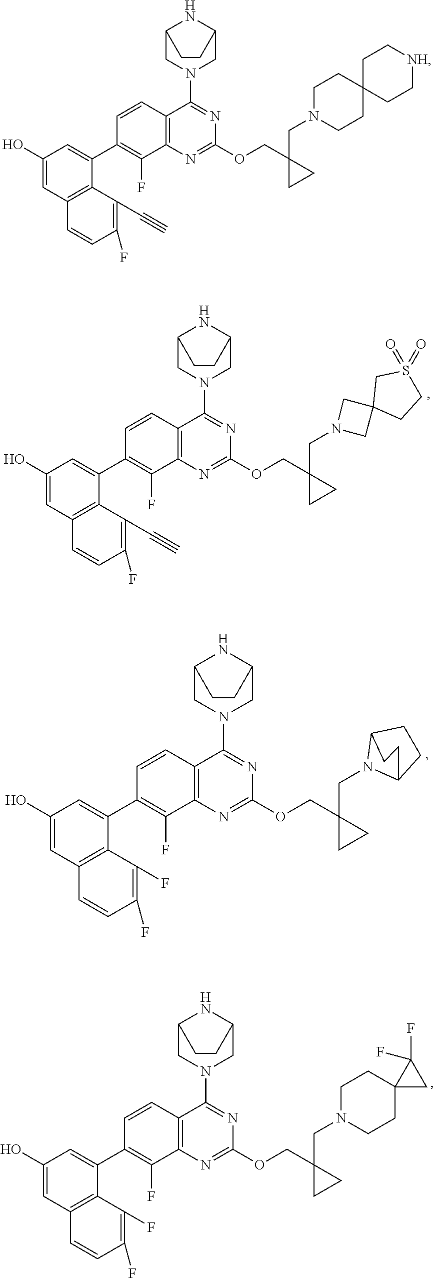 [SMILES: C#Cc1c(F)ccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CC7(CCS(=O)(=O)C7)C6)CC5)nc4c3F)c12 ; Oc1cc(-c2ccc3c(N4CC5CCC(C4)N5)nc(OCC4(CN5C6CCC5CC6)CC4)nc3c2F)c2c(F)c(F)ccc2c1 ; Oc1cc(-c2ccc3c(N4CC5CCC(C4)N5)nc(OCC4(CN5CCC6(CC5)CC6(F)F)CC4)nc3c2F)c2c(F)c(F)ccc2c1]

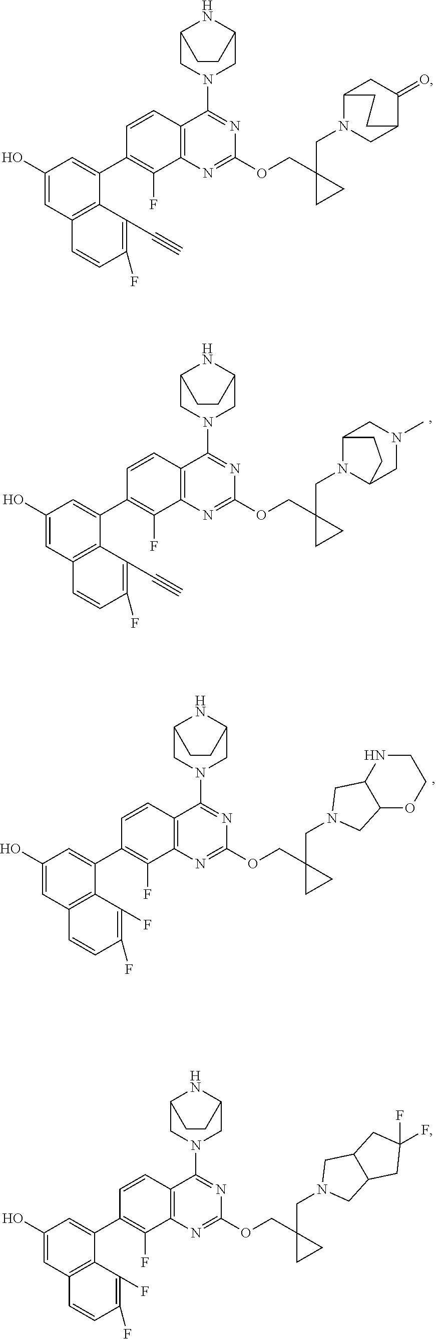 [SMILES: C#Cc1c(F)ccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6C7CCC6CN(C)C7)CC5)nc4c3F)c12 ; Oc1cc(-c2ccc3c(N4CC5CCC(C4)N5)nc(OCC4(CN5CC6NCCOC6C5)CC4)nc3c2F)c2c(F)c(F)ccc2c1 ; Oc1cc(-c2ccc3c(N4CC5CCC(C4)N5)nc(OCC4(CN5CC6CC(F)(F)CC6C5)CC4)nc3c2F)c2c(F)c(F)ccc2c1]

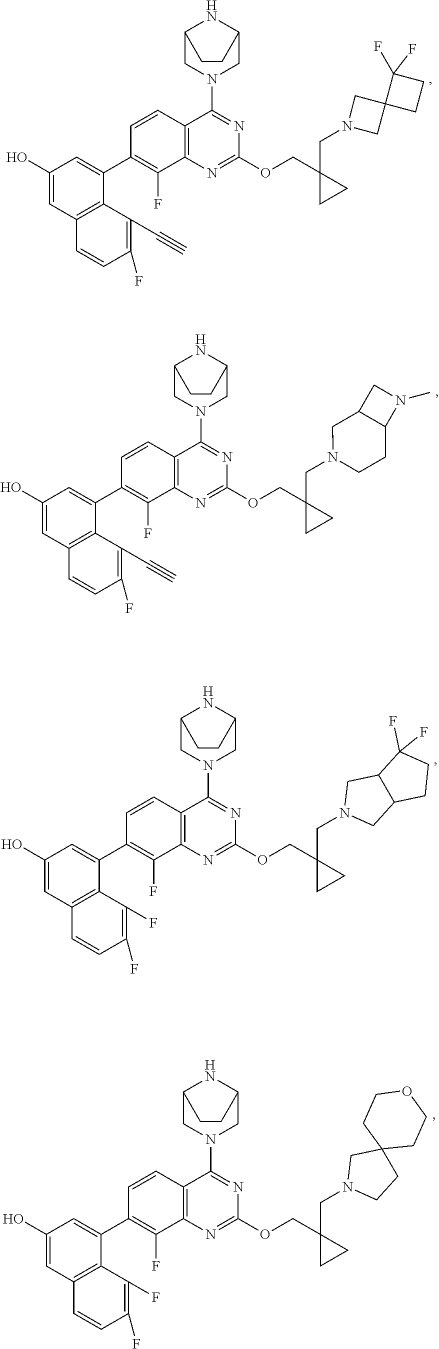 [SMILES: C#Cc1c(F)ccc2cc(O)cc(-c3ccc4c(N5CC6CCC(C5)N6)nc(OCC5(CN6CC7(CCC7(F)F)C6)CC5)nc4c3F)c12 ; C#Cc1c(F)ccc2cc(O)cc(-c3ccc4c(N5CC6CCC(C5)N6)nc(OCC5(CN6CCC7C(C6)CN7C)CC5)nc4c3F)c12 ; Oc1cc(-c2ccc3c(N4CC5CCC(C4)N5)nc(OCC4(CN5CC6CCC(F)(F)C6C5)CC4)nc3c2F)c2c(F)c(F)ccc2c1 ; Oc1cc(-c2ccc3c(N4CC5CCC(C4)N5)nc(OCC4(CN5CCC6(CCOCC6)C5)CC4)nc3c2F)c2c(F)c(F)ccc2c1]

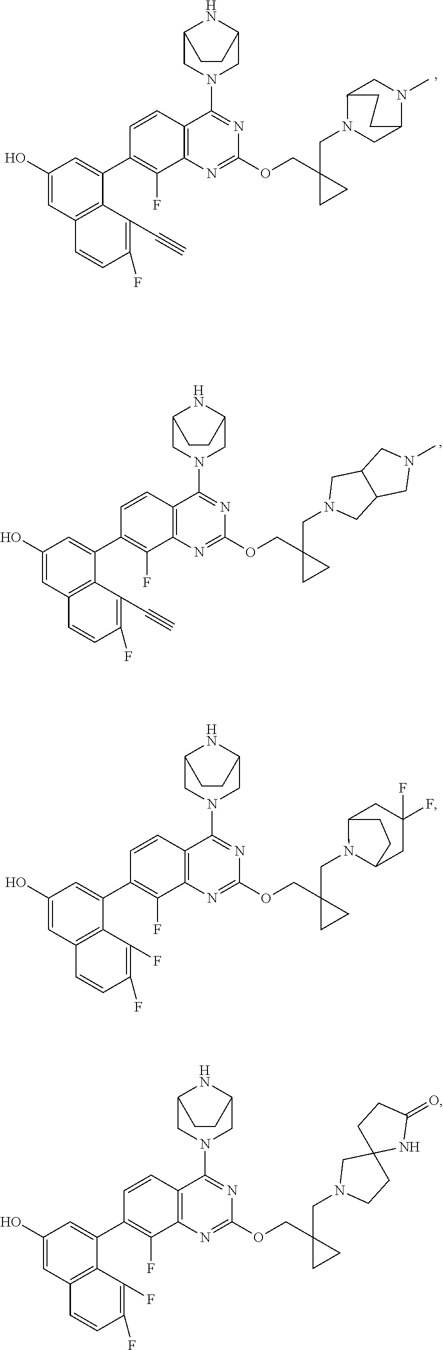 [SMILES: C#Cc1c(F)ccc2cc(O)cc(-c3ccc4c(N5CC6CCC(C5)N6)nc(OCC5(CN6CC7CCC6CN7C)CC5)nc4c3F)c12 ; C#Cc1c(F)ccc2cc(O)cc(-c3ccc4c(N5CC6CCC(C5)N6)nc(OCC5(CN6CC7CN(C)CC7C6)CC5)nc4c3F)c12 ; Oc1cc(-c2ccc3c(N4CC5CCC(C4)N5)nc(OCC4(CN5C6CCC5CC(F)(F)C6)CC4)nc3c2F)c2c(F)c(F)ccc2c1 ; O=C1CCC2(CCN(CC3(COc4nc(N5CC6CCC(C5)N6)c5ccc(-c6cc(O)cc7ccc(F)c(F)c67)c(F)c5n4)CC3)C2)N1]

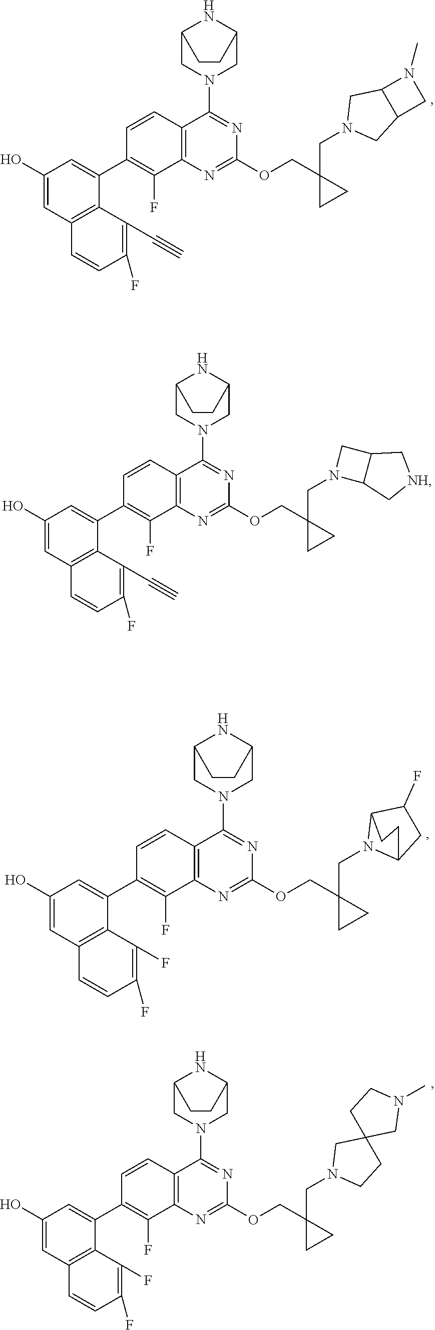 [SMILES: C#Cc1c(F)ccc2cc(O)cc(-c3ccc4c(N5CC6CCC(C5)N6)nc(OCC5(CN6CC7CN(C)C7C6)CC5)nc4c3F)c12 ; C#Cc1c(F)ccc2cc(O)cc(-c3ccc4c(N5CC6CCC(C5)N6)nc(OCC5(CN6CC7CNCC76)CC5)nc4c3F)c12 ; Oc1cc(-c2ccc3c(N4CC5CCC(C4)N5)nc(OCC4(CN5C6CCC5C(F)C6)CC4)nc3c2F)c2c(F)c(F)ccc2c1 ; CN1CCC2(CCN(CC3(COc4nc(N5CC6CCC(C5)N6)c5ccc(-c6cc(O)cc7ccc(F)c(F)c67)c(F)c5n4)CC3)C2)C1]

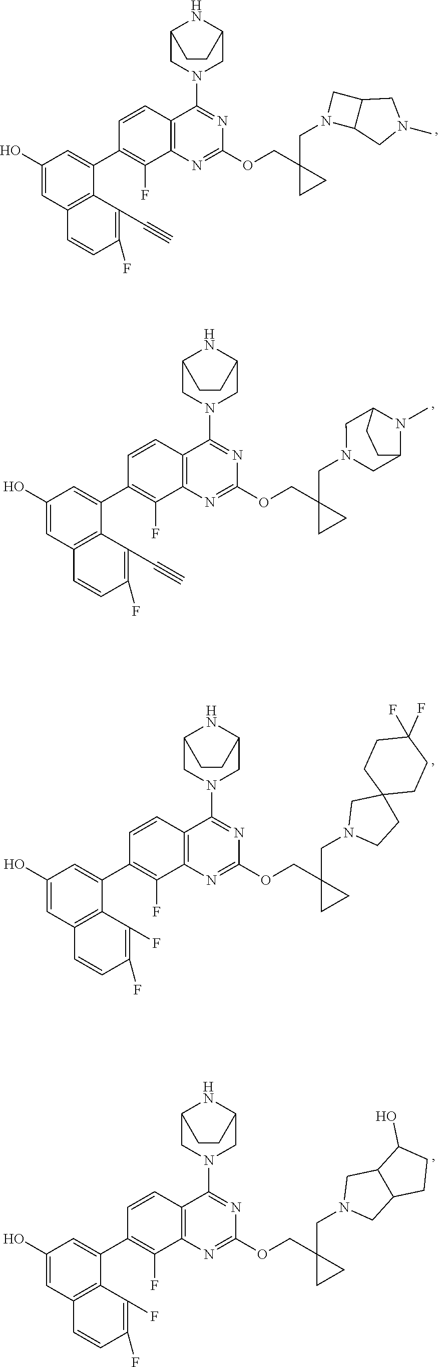 [SMILES: C#Cc1c(F)ccc2cc(O)cc(-c3ccc4c(N5CC6CCC(C5)N6)nc(OCC5(CN6CC7CN(C)CC76)CC5)nc4c3F)c12 ; C#Cc1c(F)ccc2cc(O)cc(-c3ccc4c(N5CC6CCC(C5)N6)nc(OCC5(CN6CC7CCC(C6)N7C)CC5)nc4c3F)c12 ; Oc1cc(-c2ccc3c(N4CC5CCC(C4)N5)nc(OCC4(CN5CCC6(CCC(F)(F)CC6)C5)CC4)nc3c2F)c2c(F)c(F)ccc2c1 ; Oc1cc(-c2ccc3c(N4CC5CCC(C4)N5)nc(OCC4(CN5CC6CCC(O)C6C5)CC4)nc3c2F)c2c(F)c(F)ccc2c1]

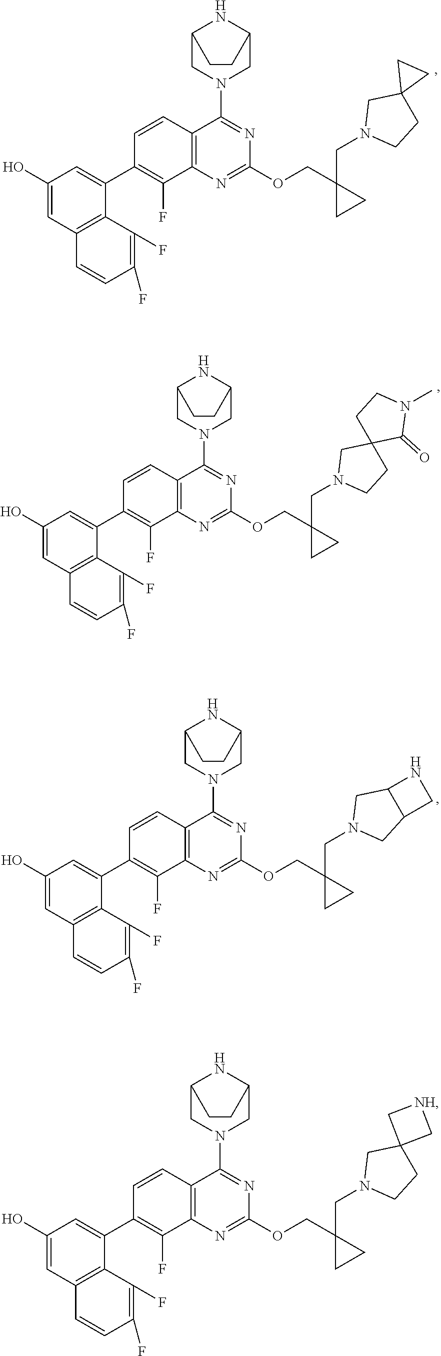 [SMILES: Oc1cc(-c2ccc3c(N4CC5CCC(C4)N5)nc(OCC4(CN5CCC6(CC6)C5)CC4)nc3c2F)c2c(F)c(F)ccc2c1 ; CN1CCC2(CCN(CC3(COc4nc(N5CC6CCC(C5)N6)c5ccc(-c6cc(O)cc7ccc(F)c(F)c67)c(F)c5n4)CC3)C2)C1=O ; Oc1cc(-c2ccc3c(N4CC5CCC(C4)N5)nc(OCC4(CN5CC6CNC6C5)CC4)nc3c2F)c2c(F)c(F)ccc2c1 ; Oc1cc(-c2ccc3c(N4CC5CCC(C4)N5)nc(OCC4(CN5CCC6(CNC6)C5)CC4)nc3c2F)c2c(F)c(F)ccc2c1]

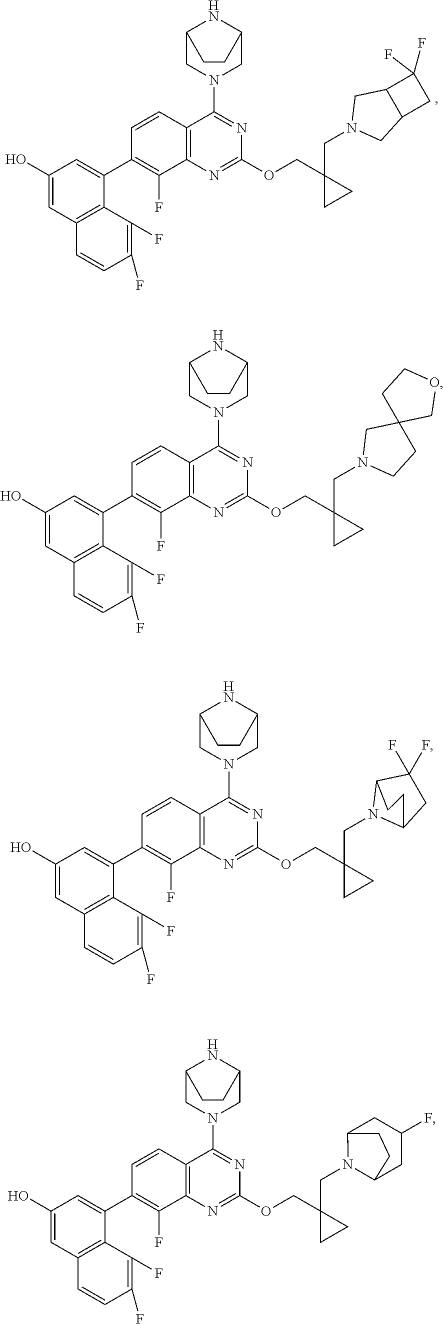 [SMILES: Oc1cc(-c2ccc3c(N4CC5CCC(C4)N5)nc(OCC4(CN5CC6CC(F)(F)C6C5)CC4)nc3c2F)c2c(F)c(F)ccc2c1 ; Oc1cc(-c2ccc3c(N4CC5CCC(C4)N5)nc(OCC4(CN5CCC6(CCOC6)C5)CC4)nc3c2F)c2c(F)c(F)ccc2c1 ; Oc1cc(-c2ccc3c(N4CC5CCC(C4)N5)nc(OCC4(CN5C6CCC5C(F)(F)C6)CC4)nc3c2F)c2c(F)c(F)ccc2c1 ; Oc1cc(-c2ccc3c(N4CC5CCC(C4)N5)nc(OCC4(CN5C6CCC5CC(F)C6)CC4)nc3c2F)c2c(F)c(F)ccc2c1]

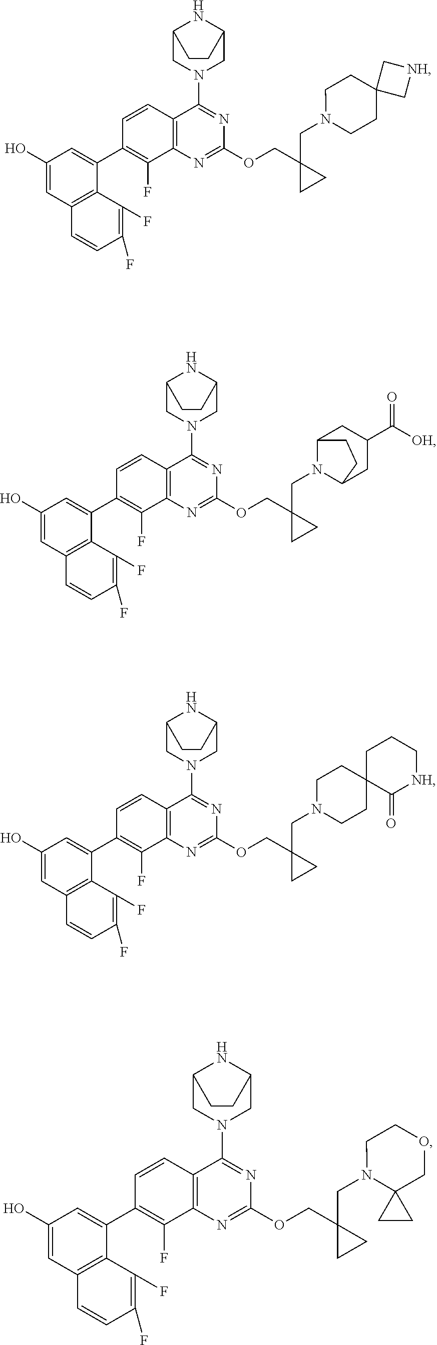 [SMILES: Oc1cc(-c2ccc3c(N4CC5CCC(C4)N5)nc(OCC4(CN5CCC6(CC5)CNC6)CC4)nc3c2F)c2c(F)c(F)ccc2c1 ; O=C(O)C1CC2CCC(C1)N2CC1(COc2nc(N3CC4CCC(C3)N4)c3ccc(-c4cc(O)cc5ccc(F)c(F)c45)c(F)c3n2)CC1 ; O=C1NCCCC12CCN(CC1(COc3nc(N4CC5CCC(C4)N5)c4ccc(-c5cc(O)cc6ccc(F)c(F)c56)c(F)c4n3)CC1)CC2 ; Oc1cc(-c2ccc3c(N4CC5CCC(C4)N5)nc(OCC4(CN5CCOCC56CC6)CC4)nc3c2F)c2c(F)c(F)ccc2c1]

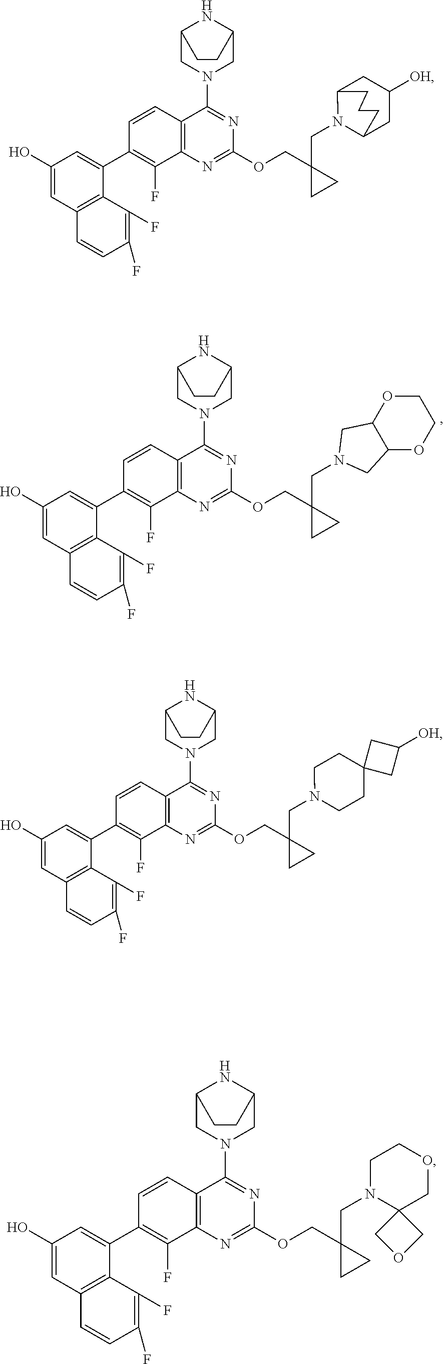 [SMILES: Oc1cc(-c2ccc3c(N4CC5CCC(C4)N5)nc(OCC4(CN5C6CCCC5CC(O)C6)CC4)nc3c2F)c2c(F)c(F)ccc2c1 ; Oc1cc(-c2ccc3c(N4CC5CCC(C4)N5)nc(OCC4(CN5CC6OCCOC6C5)CC4)nc3c2F)c2c(F)c(F)ccc2c1 ; Oc1cc(-c2ccc3c(N4CC5CCC(C4)N5)nc(OCC4(CN5CCC6(CC5)CC(O)C6)CC4)nc3c2F)c2c(F)c(F)ccc2c1 ; Oc1cc(-c2ccc3c(N4CC5CCC(C4)N5)nc(OCC4(CN5CCOCC56COC6)CC4)nc3c2F)c2c(F)c(F)ccc2c1]

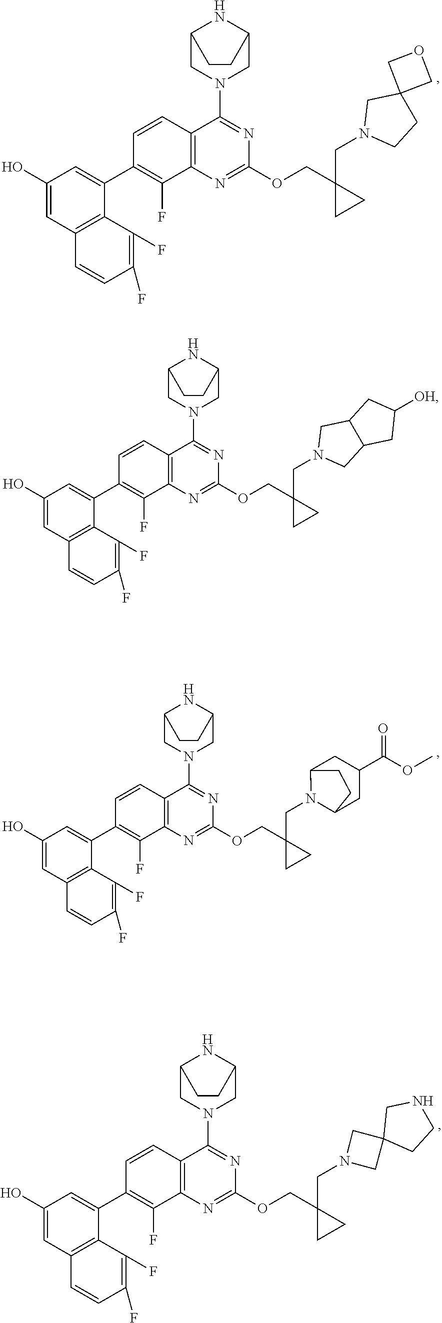 [SMILES: Oc1cc(-c2ccc3c(N4CC5CCC(C4)N5)nc(OCC4(CN5CCC6(COC6)C5)CC4)nc3c2F)c2c(F)c(F)ccc2c1 ; Oc1cc(-c2ccc3c(N4CC5CCC(C4)N5)nc(OCC4(CN5CC6CC(O)CC6C5)CC4)nc3c2F)c2c(F)c(F)ccc2c1 ; COC(=O)C1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCNC6)C5)CC4)nc3c2F)c2c(F)c(F)ccc2c1]

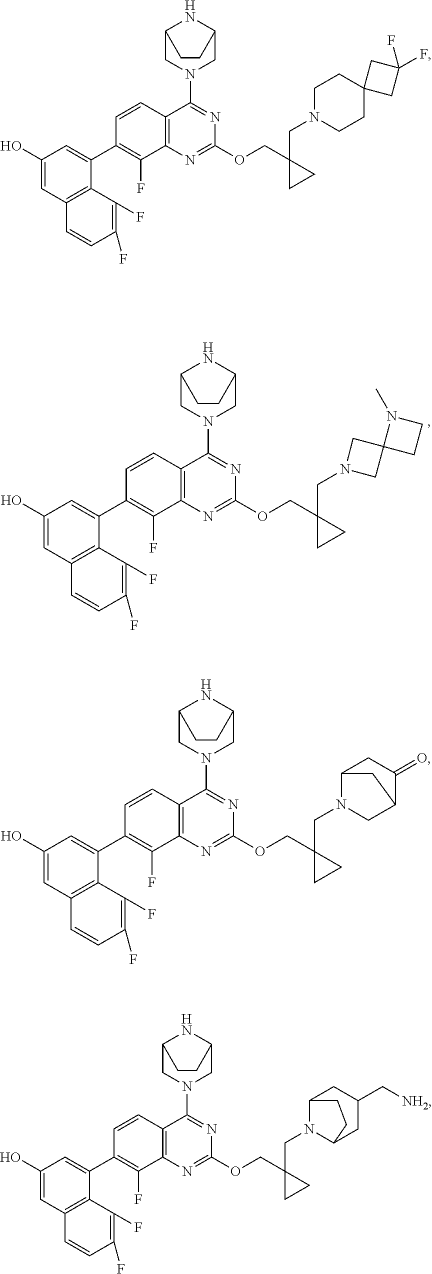 [SMILES: Oc1cc(-c2ccc3c(N4CC5CCC(C4)N5)nc(OCC4(CN5CCC6(CC5)CC(F)(F)C6)CC4)nc3c2F)c2c(F)c(F)ccc2c1 ; CN1CCC12CN(CC1(COc3nc(N4CC5CCC(C4)N5)c4ccc(-c5cc(O)cc6ccc(F)c(F)c56)c(F)c4n3)CC1)C2 ; O=C1CC2CC1CN2CC1(COc2nc(N3CC4CCC(C3)N4)c3ccc(-c4cc(O)cc5ccc(F)c(F)c45)c(F)c3n2)CC1 ; NCC1CC2CCC(C1)N2CC1(COc2nc(N3CC4CCC(C3)N4)c3ccc(-c4cc(O)cc5ccc(F)c(F)c45)c(F)c3n2)CC1]

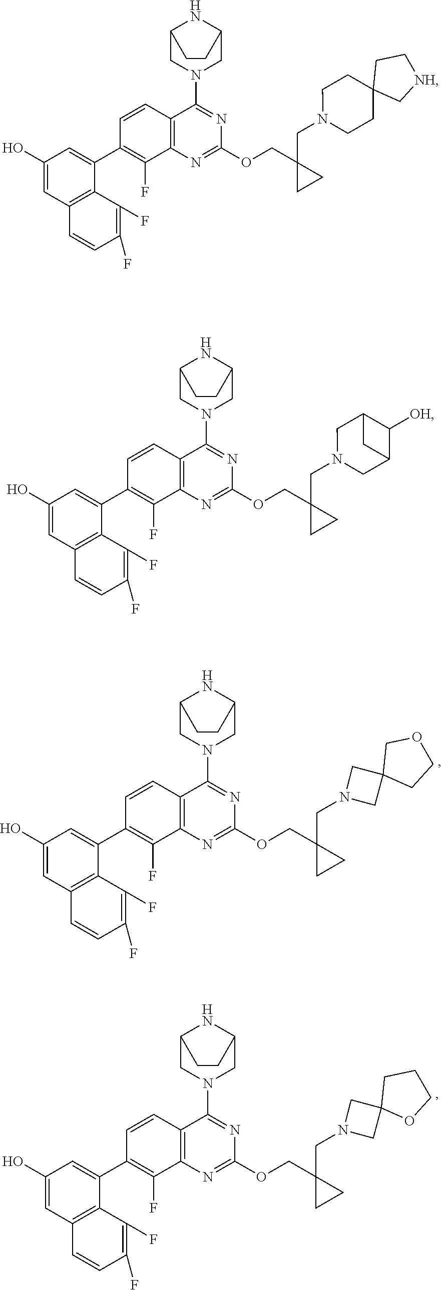 [SMILES: Oc1cc(-c2ccc3c(N4CC5CCC(C4)N5)nc(OCC4(CN5CCC6(CCNC6)CC5)CC4)nc3c2F)c2c(F)c(F)ccc2c1 ; Oc1cc(-c2ccc3c(N4CC5CCC(C4)N5)nc(OCC4(CN5CC6CC(C5)C6O)CC4)nc3c2F)c2c(F)c(F)ccc2c1 ; Oc1cc(-c2ccc3c(N4CC5CCC(C4)N5)nc(OCC4(CN5CC6(CCOC6)C5)CC4)nc3c2F)c2c(F)c(F)ccc2c1 ; Oc1cc(-c2ccc3c(N4CC5CCC(C4)N5)nc(OCC4(CN5CC6(CCCO6)C5)CC4)nc3c2F)c2c(F)c(F)ccc2c1]

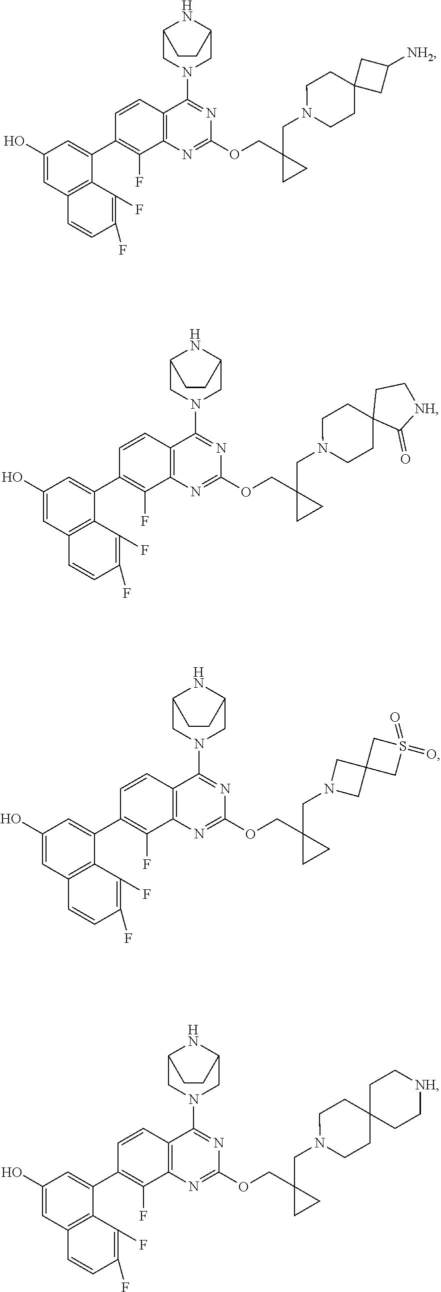 [SMILES: NC1CC2(CCN(CC3(COc4nc(N5CC6CCC(C5)N6)c5ccc(-c6cc(O)cc7ccc(F)c(F)c67)c(F)c5n4)CC3)CC2)C1 ; O=C1NCCC12CCN(CC1(COc3nc(N4CC5CCC(C4)N5)c4ccc(-c5cc(O)cc6ccc(F)c(F)c56)c(F)c4n3)CC1)CC2 ; O=S1(=O)CC2(CN(CC3(COc4nc(N5CC6CCC(C5)N6)c5ccc(-c6cc(O)cc7ccc(F)c(F)c67)c(F)c5n4)CC3)C2)C1 ; Oc1cc(-c2ccc3c(N4CC5CCC(C4)N5)nc(OCC4(CN5CCC6(CCNCC6)CC5)CC4)nc3c2F)c2c(F)c(F)ccc2c1]

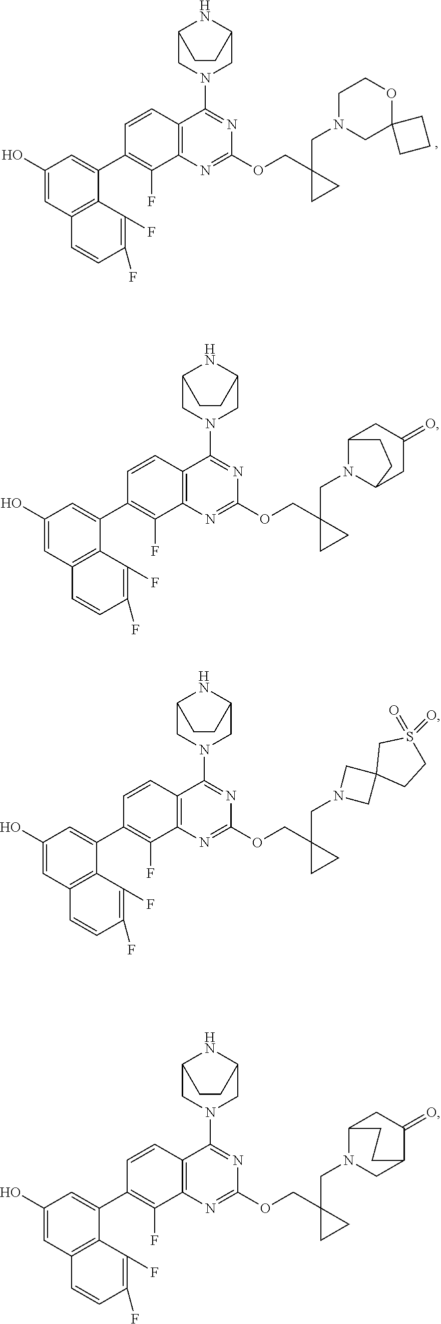 [SMILES: Oc1cc(-c2ccc3c(N4CC5CCC(C4)N5)nc(OCC4(CN5CCOC6(CCC6)C5)CC4)nc3c2F)c2c(F)c(F)ccc2c1 ; O=C1CC2CCC(C1)N2CC1(COc2nc(N3CC4CCC(C3)N4)c3ccc(-c4cc(O)cc5ccc(F)c(F)c45)c(F)c3n2)CC1 ; O=S1(=O)CCC2(CN(CC3(COc4nc(N5CC6CCC(C5)N6)c5ccc(-c6cc(O)cc7ccc(F)c(F)c67)c(F)c5n4)CC3)C2)C1 ; O=C1CC2CCC1CN2CC1(COc2nc(N3CC4CCC(C3)N4)c3ccc(-c4cc(O)cc5ccc(F)c(F)c45)c(F)c3n2)CC1]

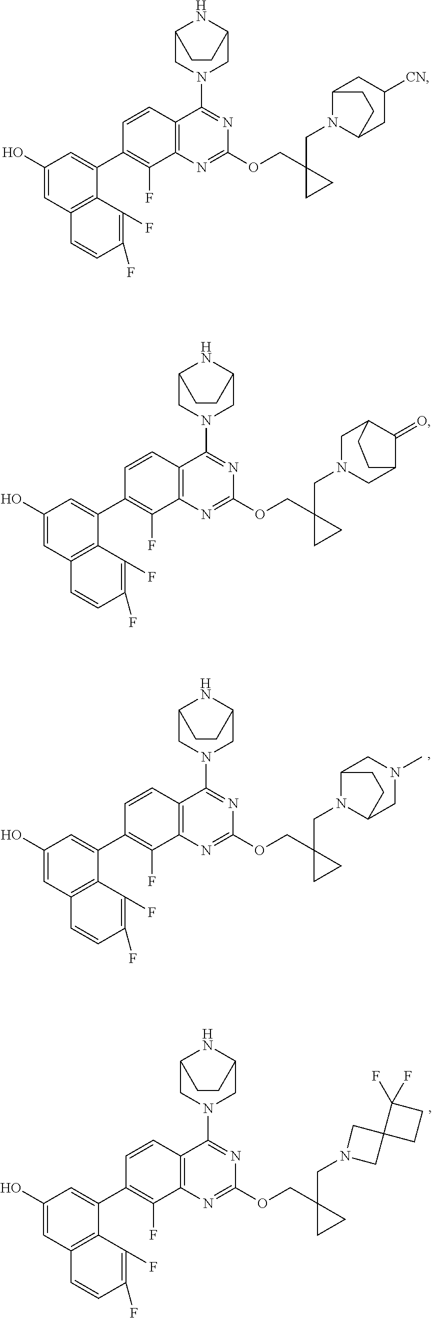 [SMILES: N#CC1CC2CCC(C1)N2CC1(COc2nc(N3CC4CCC(C3)N4)c3ccc(-c4cc(O)cc5ccc(F)c(F)c45)c(F)c3n2)CC1 ; O=C1C2CCC1CN(CC1(COc3nc(N4CC5CCC(C4)N5)c4ccc(-c5cc(O)cc6ccc(F)c(F)c56)c(F)c4n3)CC1)C2 ; CN1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCC6(F)F)C5)CC4)nc3c2F)c2c(F)c(F)ccc2c1]

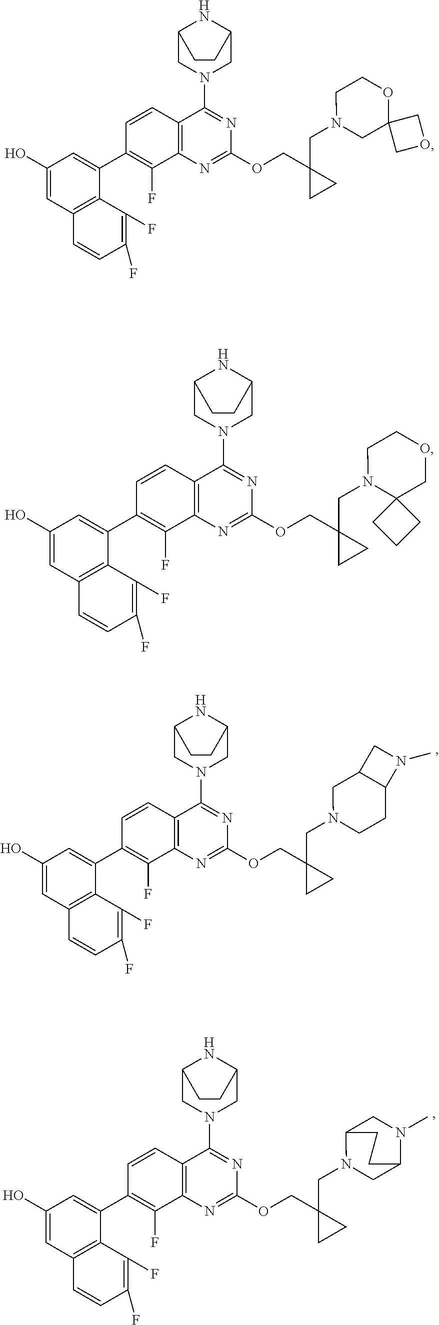 [SMILES: Oc1cc(-c2ccc3c(N4CC5CCC(C4)N5)nc(OCC4(CN5CCOC6(COC6)C5)CC4)nc3c2F)c2c(F)c(F)ccc2c1 ; Oc1cc(-c2ccc3c(N4CC5CCC(C4)N5)nc(OCC4(CN5CCOCC56CCC6)CC4)nc3c2F)c2c(F)c(F)ccc2c1 ; CN1CC2CN(CC3(COc4nc(N5CC6CCC(C5)N6)c5ccc(-c6cc(O)cc7ccc(F)c(F)c67)c(F)c5n4)CC3)CCC21 ; CN1CC2CCC1CN2CC1(COc2nc(N3CC4CCC(C3)N4)c3ccc(-c4cc(O)cc5ccc(F)c(F)c45)c(F)c3n2)CC1]

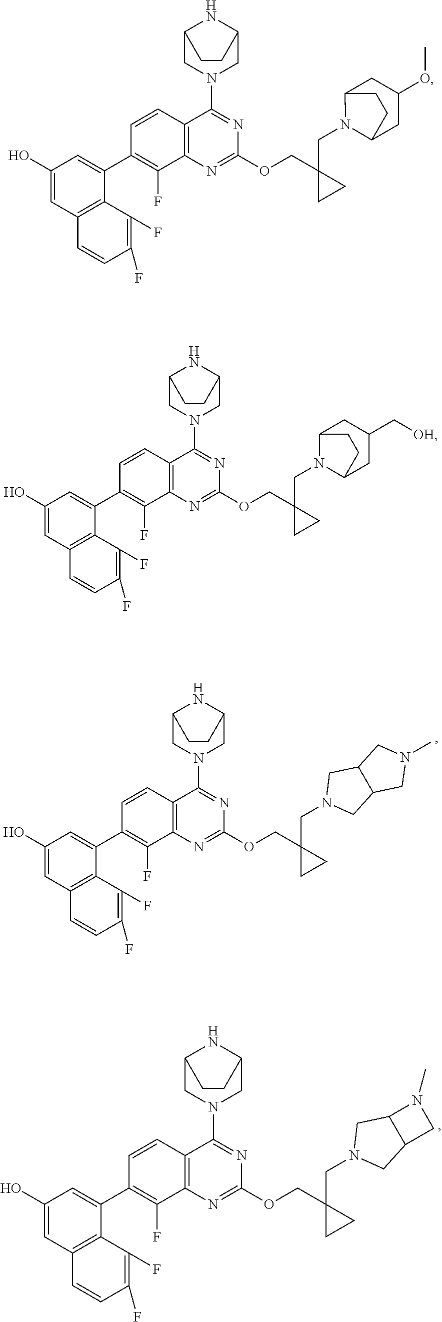 [SMILES: COC1CC2CCC(C1)N2CC1(COc2nc(N3CC4CCC(C3)N4)c3ccc(-c4cc(O)cc5ccc(F)c(F)c45)c(F)c3n2)CC1 ; OCC1CC2CCC(C1)N2CC1(COc2nc(N3CC4CCC(C3)N4)c3ccc(-c4cc(O)cc5ccc(F)c(F)c45)c(F)c3n2)CC1 ; CN1CC2CN(CC3(COc4nc(N5CC6CCC(C5)N6)c5ccc(-c6cc(O)cc7ccc(F)c(F)c67)c(F)c5n4)CC3)CC2C1 ; CN1CC2CN(CC3(COc4nc(N5CC6CCC(C5)N6)c5ccc(-c6cc(O)cc7ccc(F)c(F)c67)c(F)c5n4)CC3)CC21]

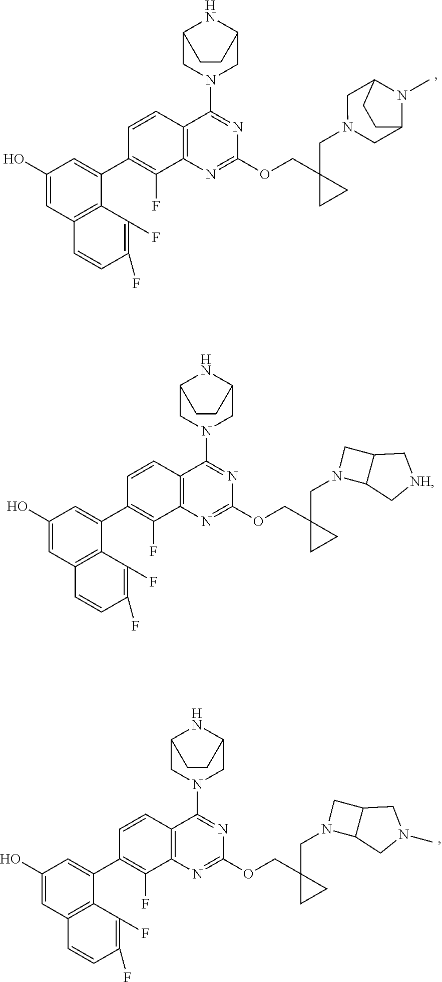 [SMILES: CN1C2CCC1CN(CC1(COc3nc(N4CC5CCC(C4)N5)c4ccc(-c5cc(O)cc6ccc(F)c(F)c56)c(F)c4n3)CC1)C2 ; Oc1cc(-c2ccc3c(N4CC5CCC(C4)N5)nc(OCC4(CN5CC6CNCC65)CC4)nc3c2F)c2c(F)c(F)ccc2c1 ; CN1CC2CN(CC3(COc4nc(N5CC6CCC(C5)N6)c5ccc(-c6cc(O)cc7ccc(F)c(F)c67)c(F)c5n4)CC3)C2C1]

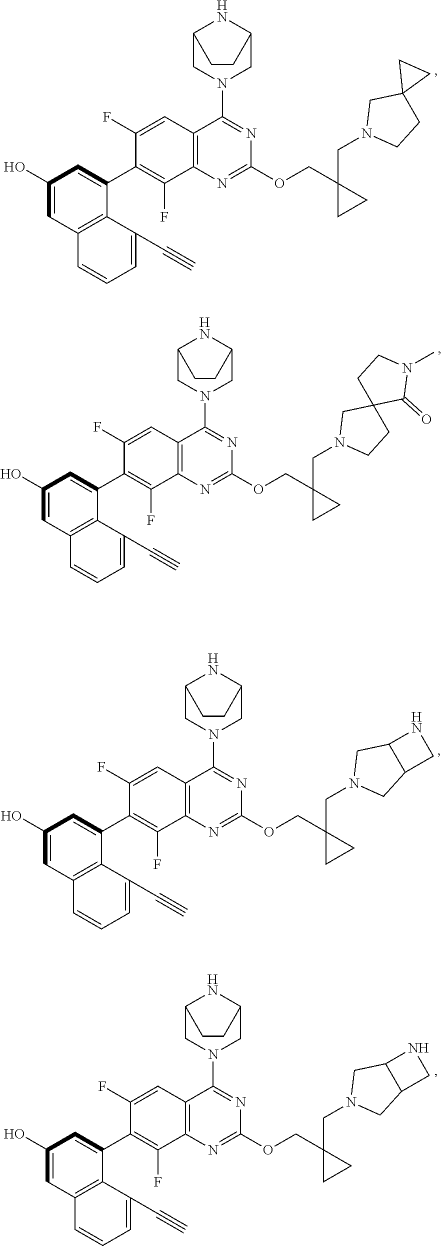

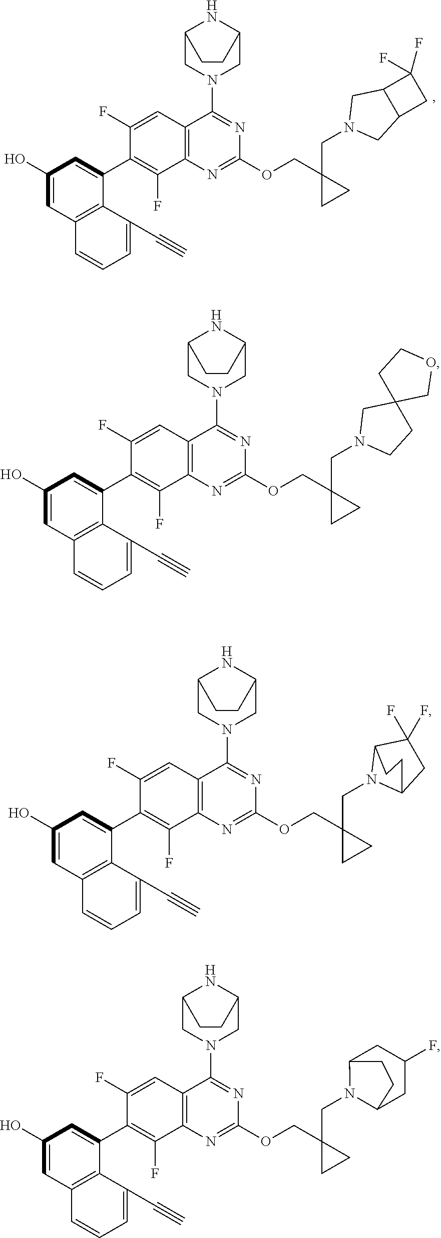

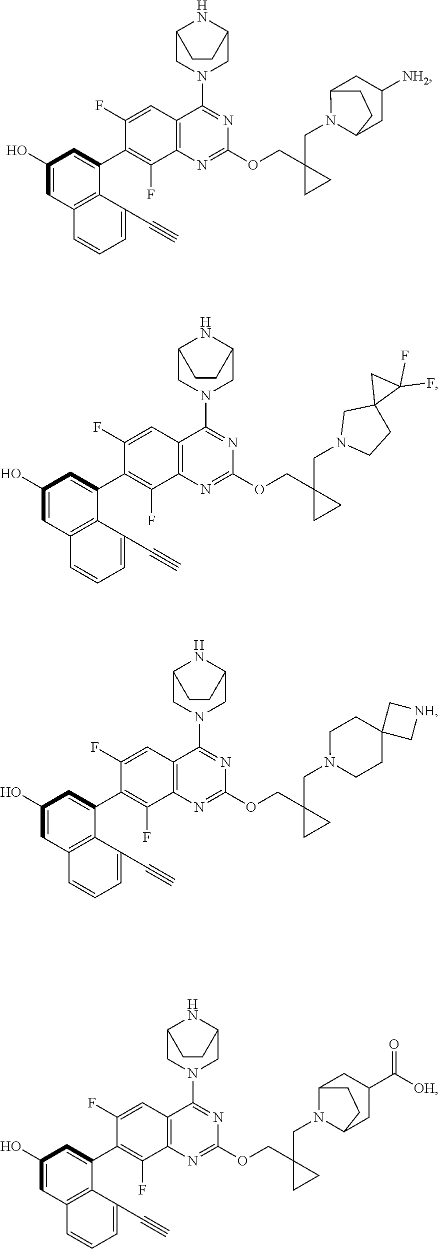

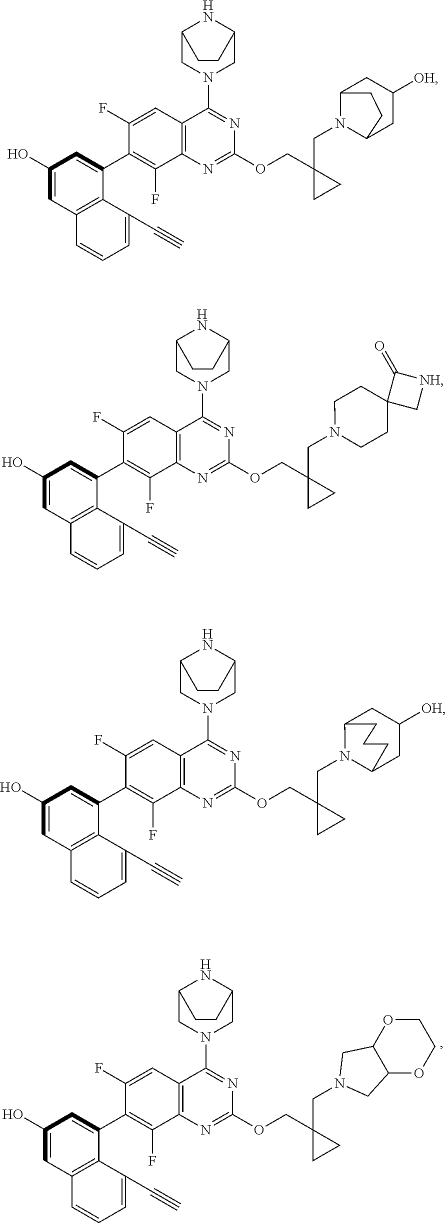

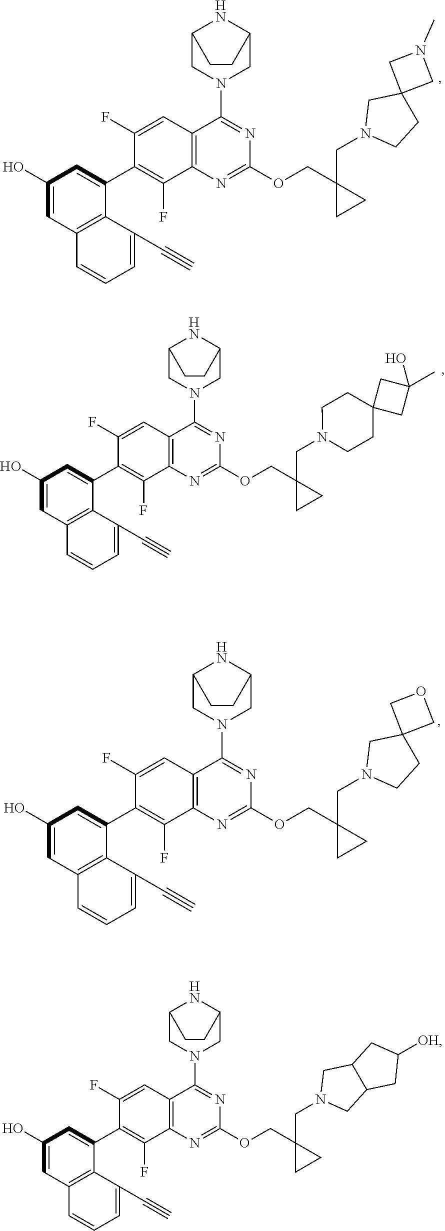

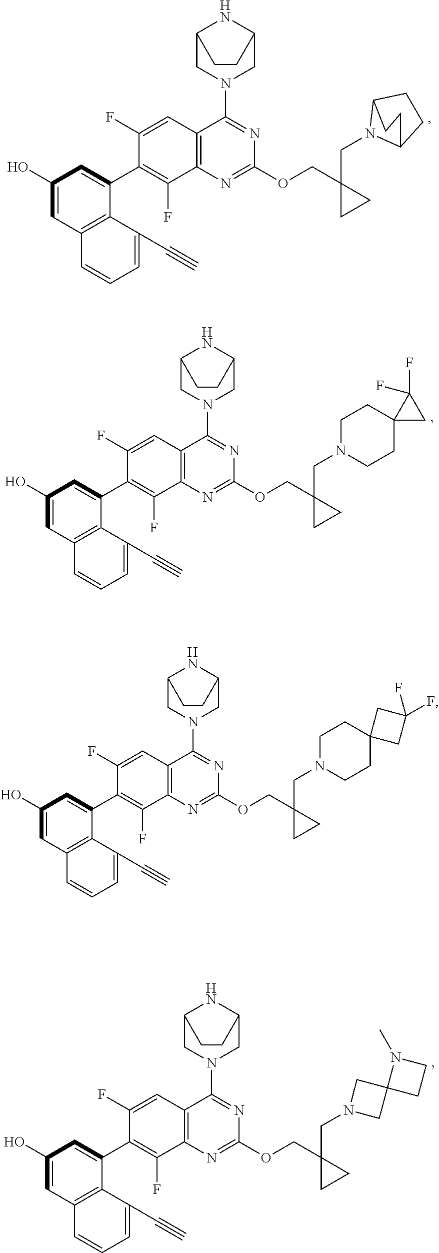

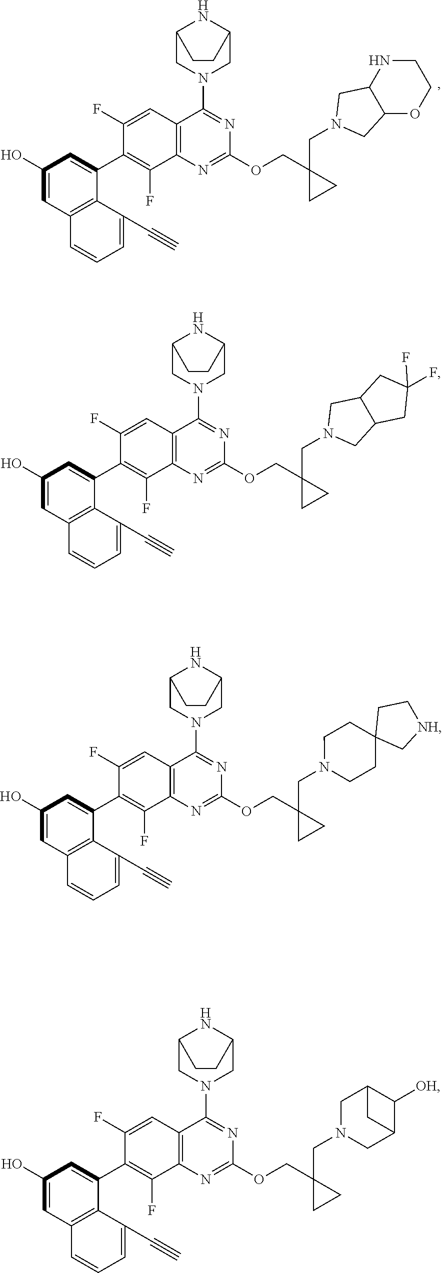

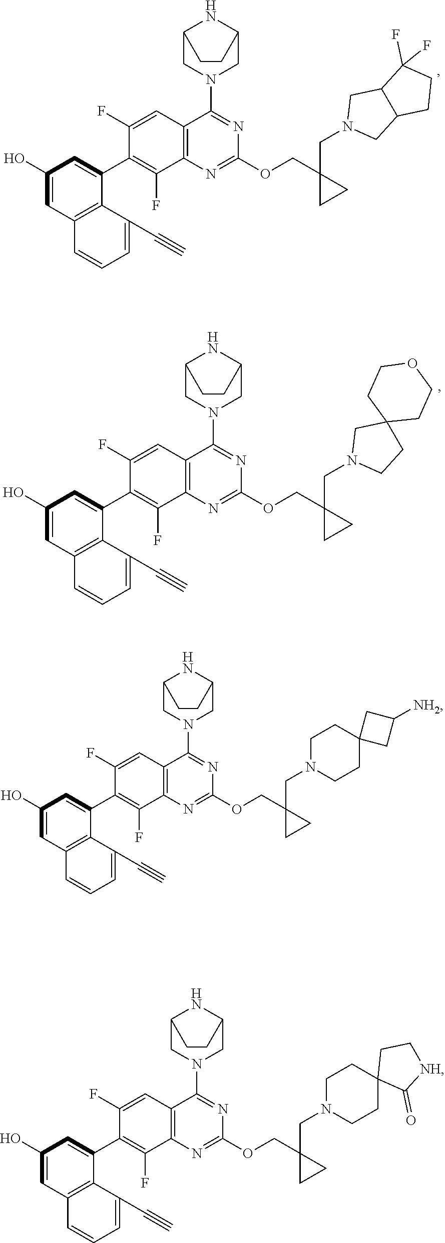

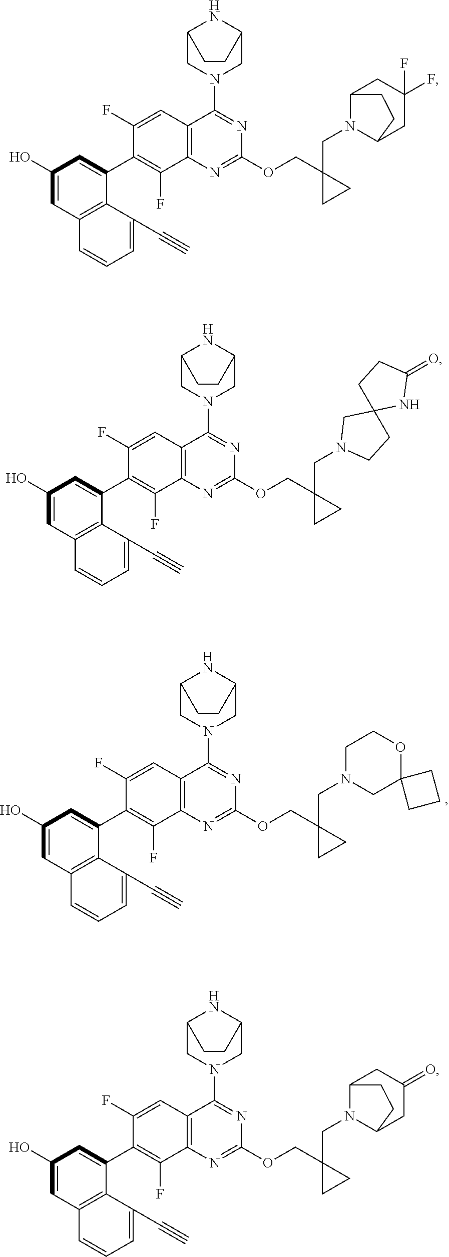

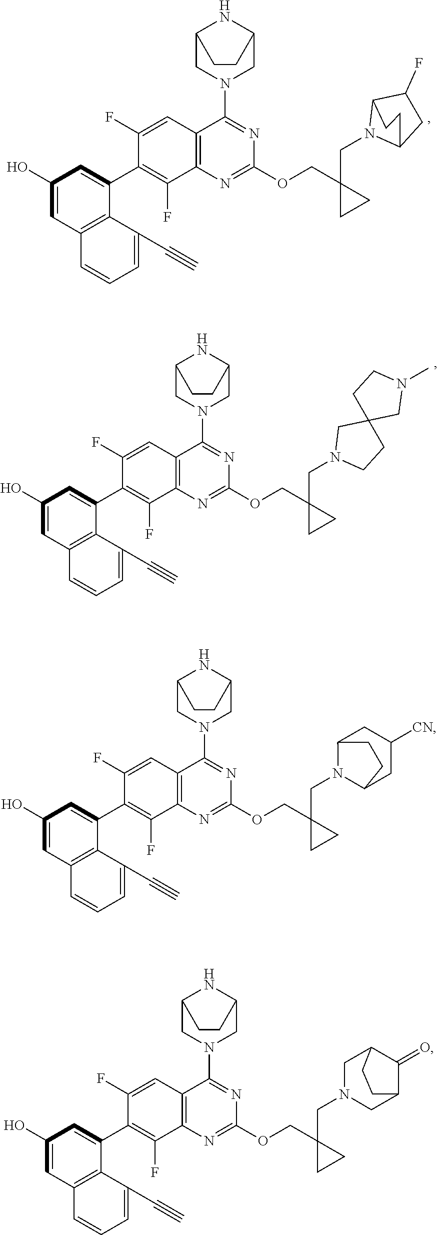

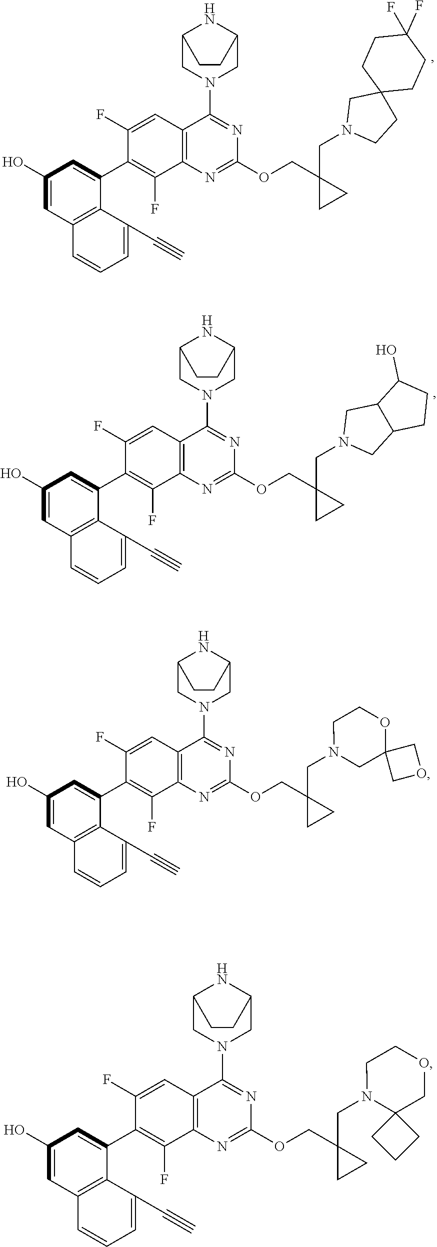

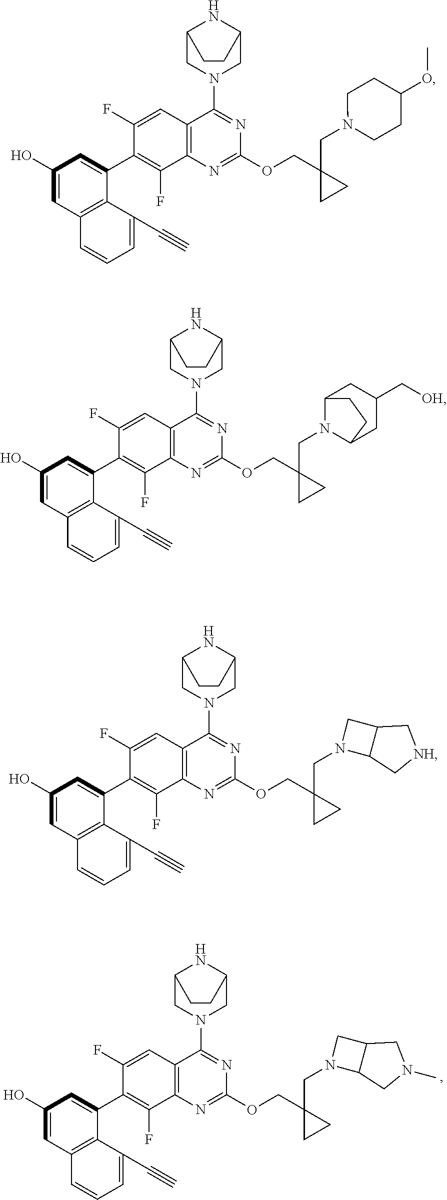

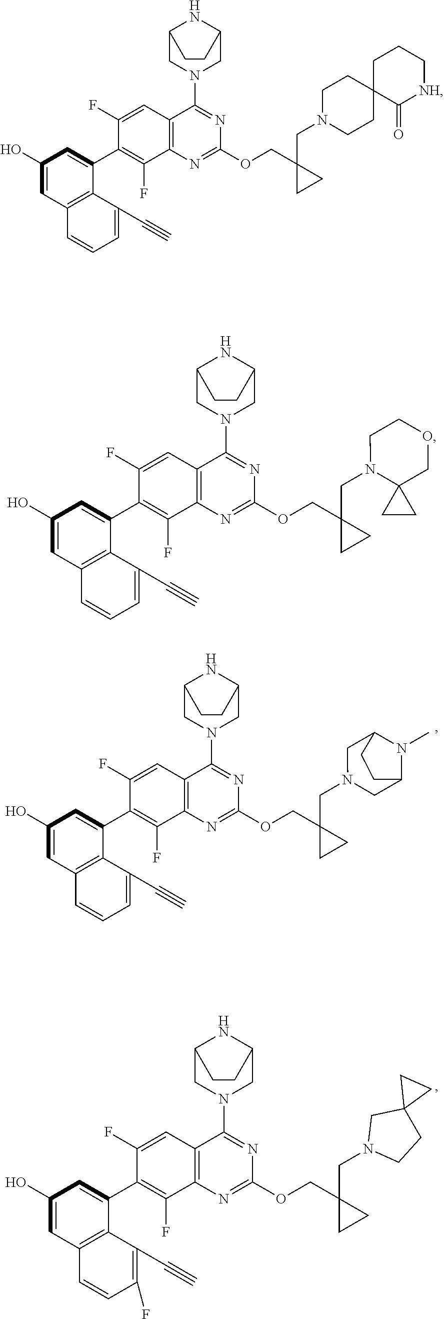

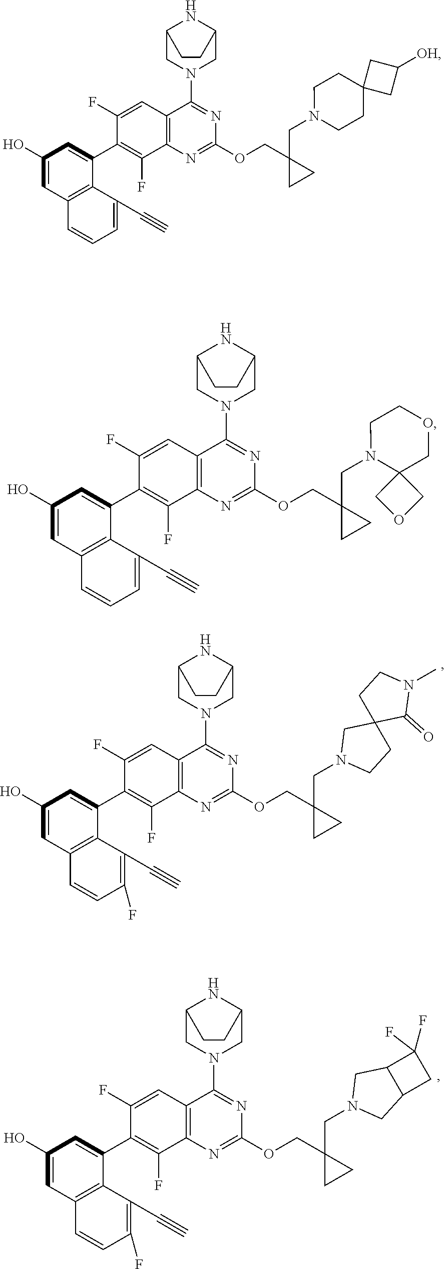

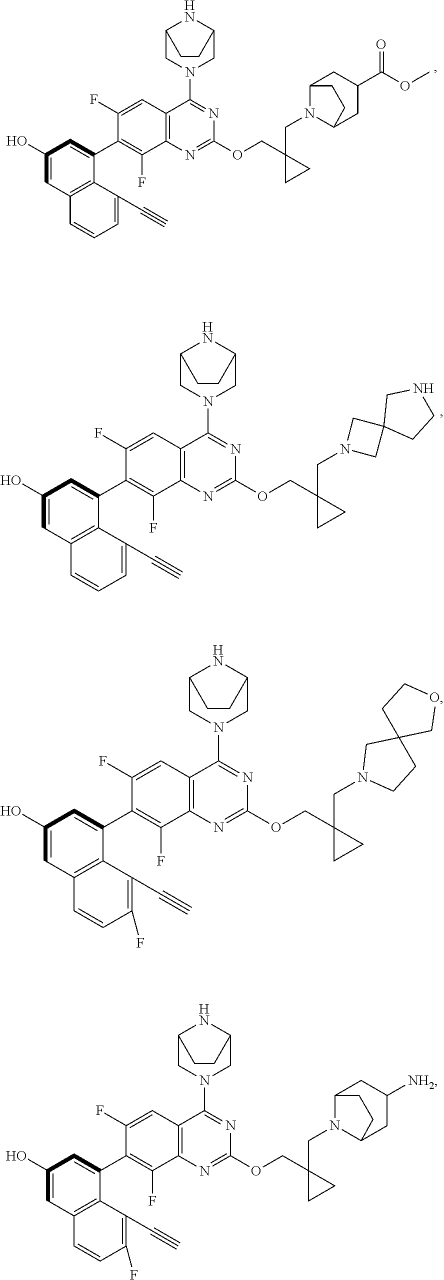

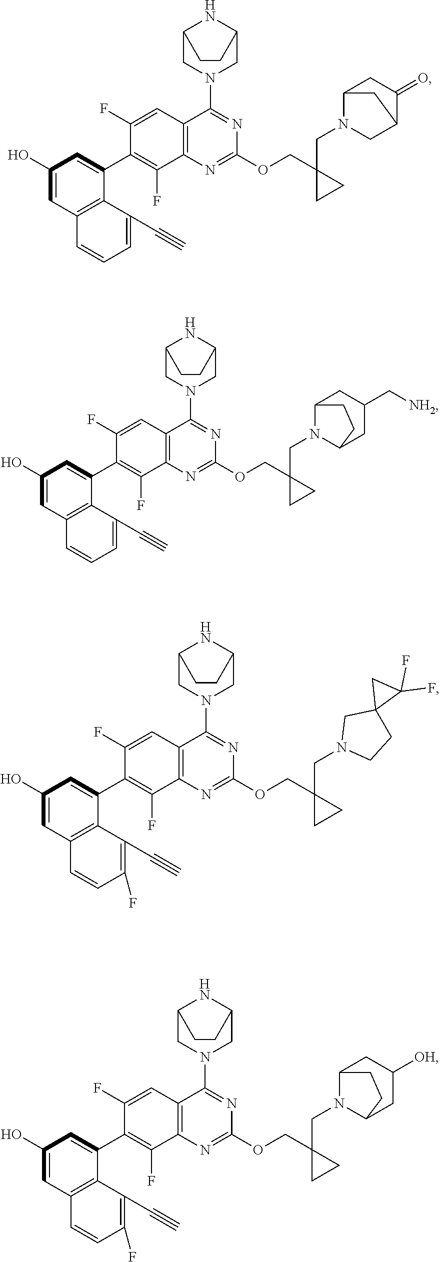

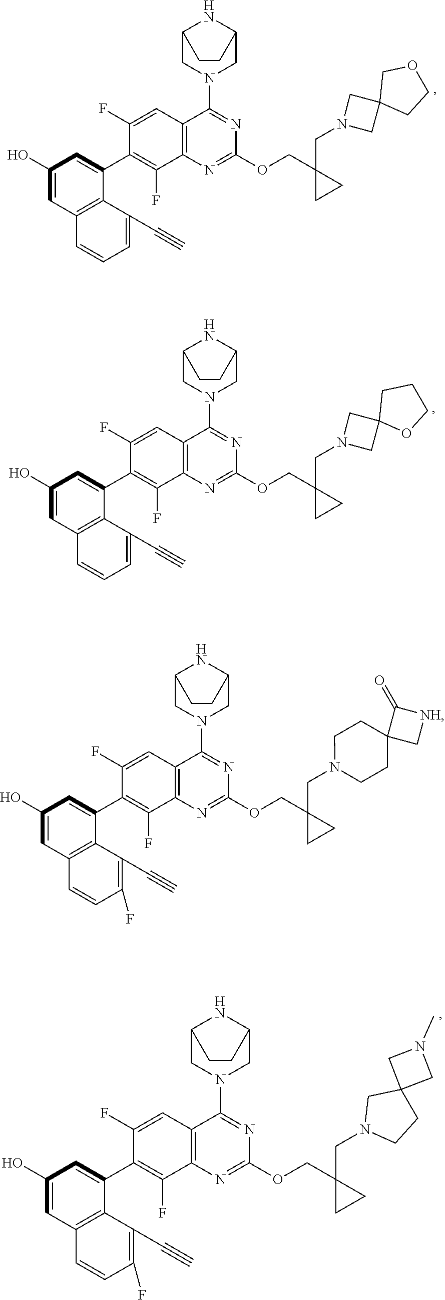

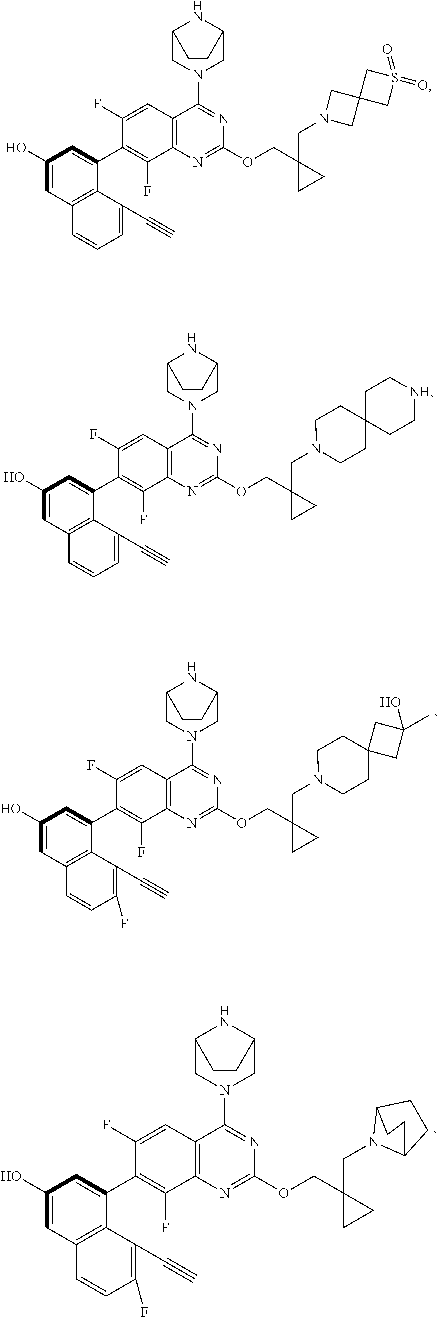

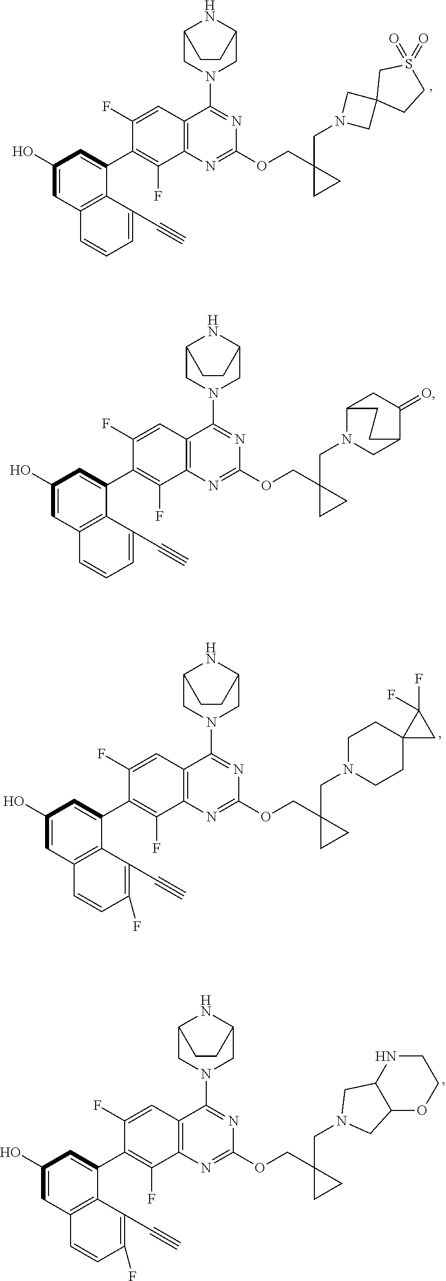

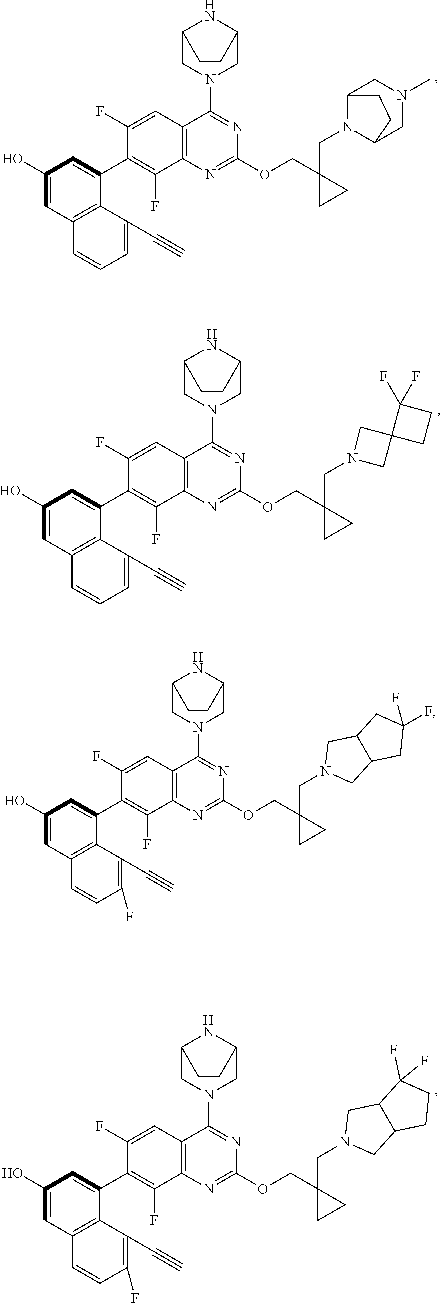

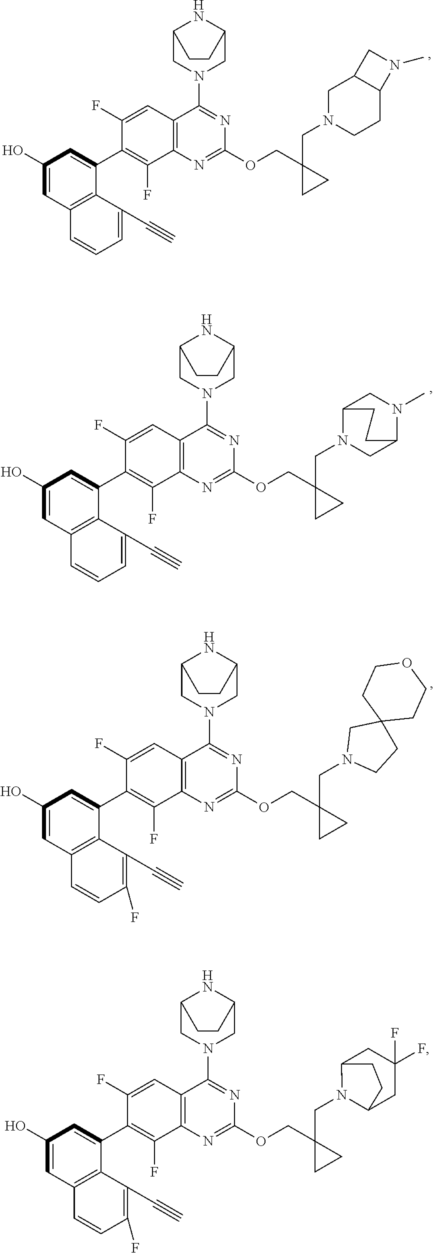

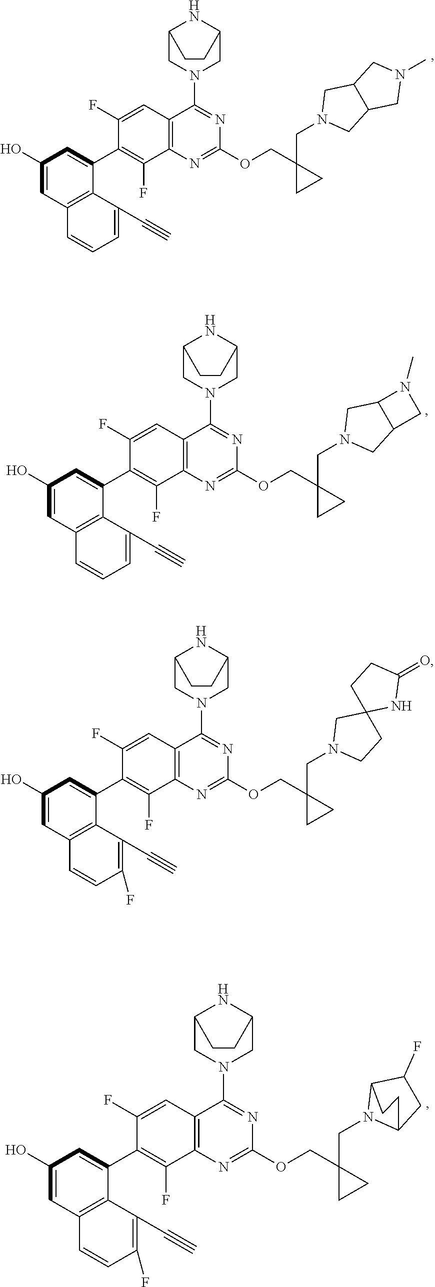

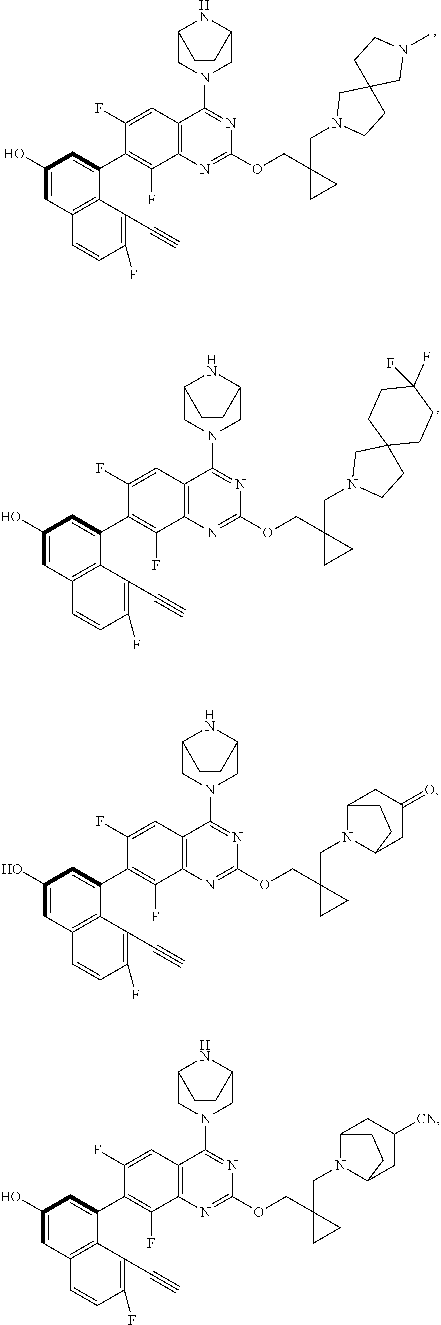

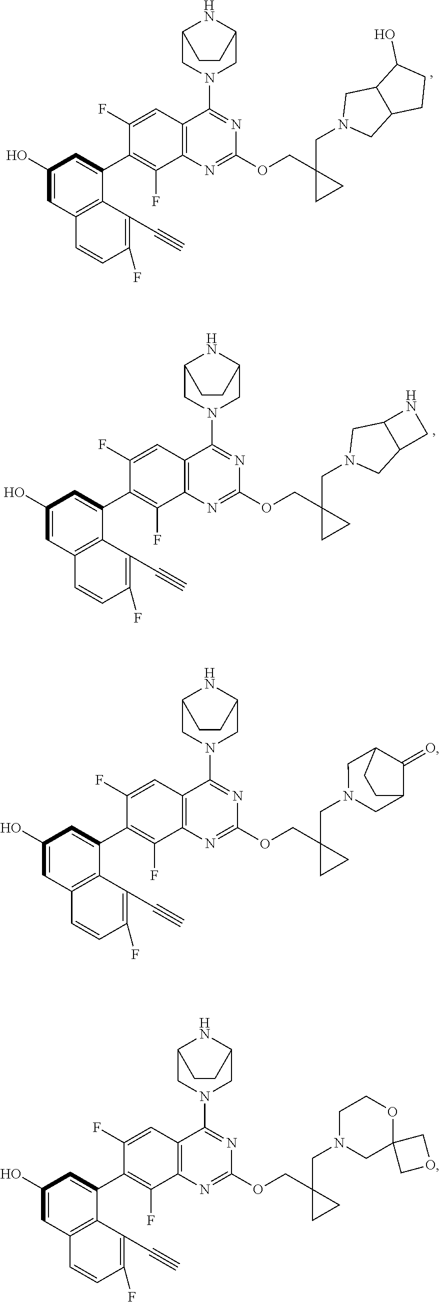

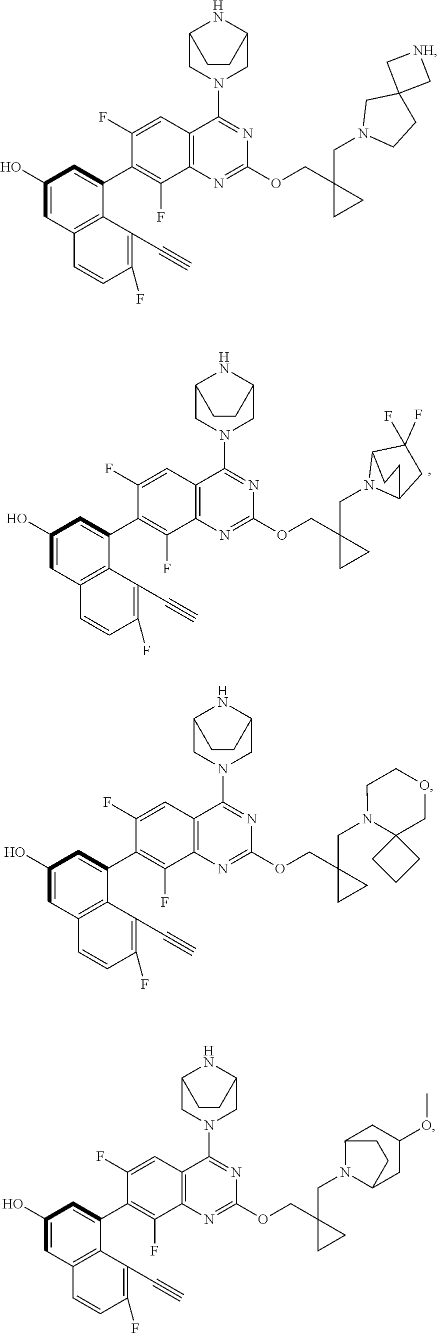

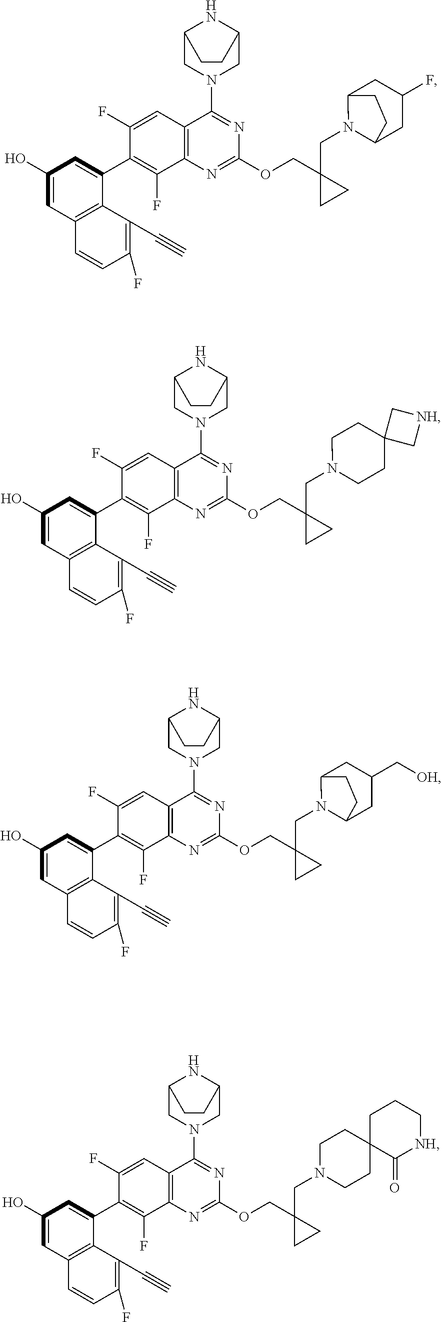

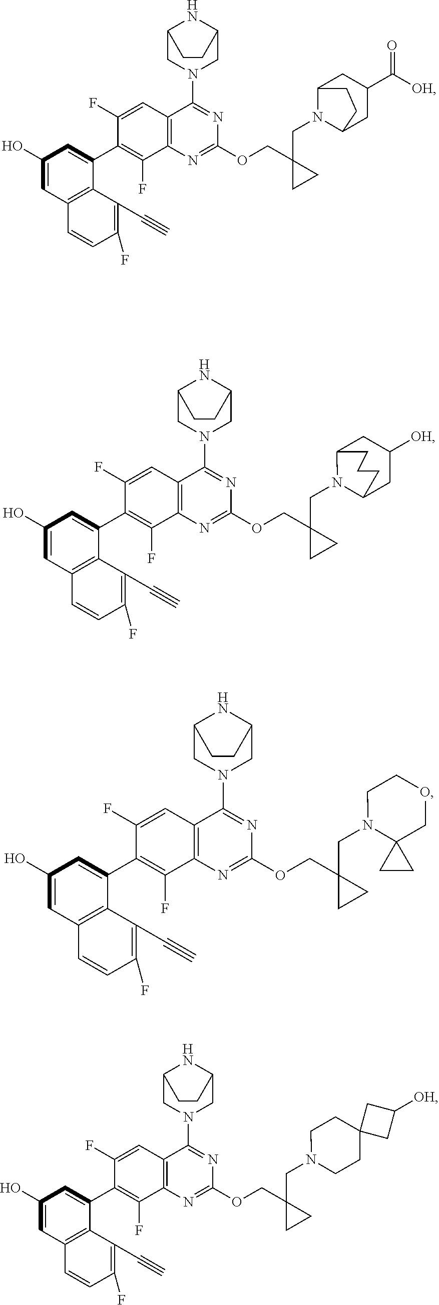

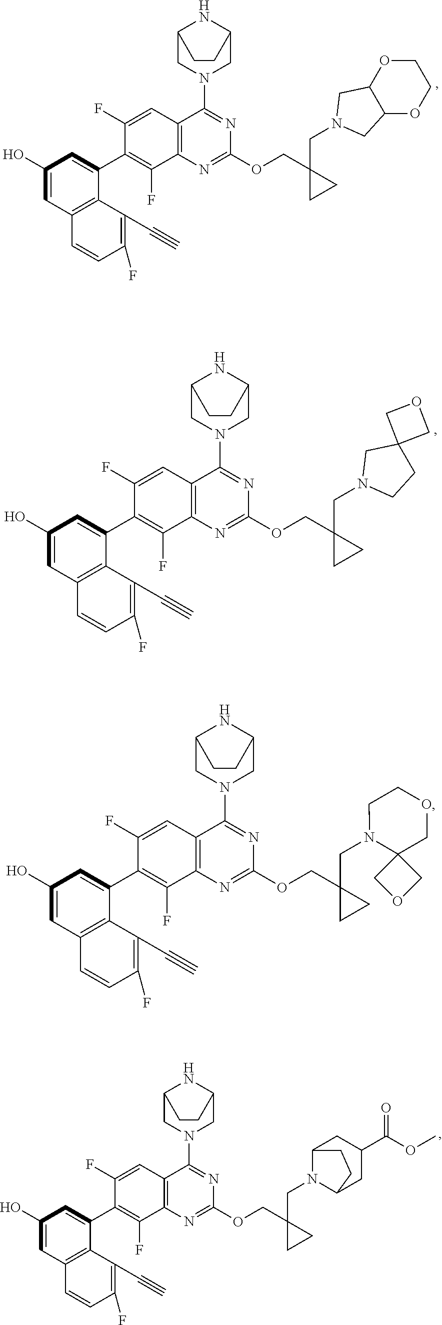

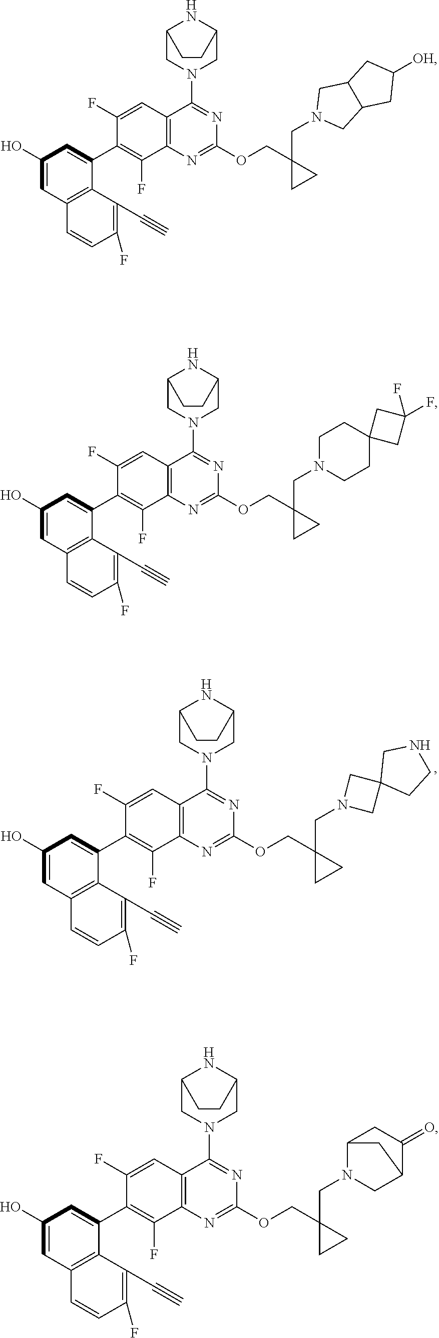

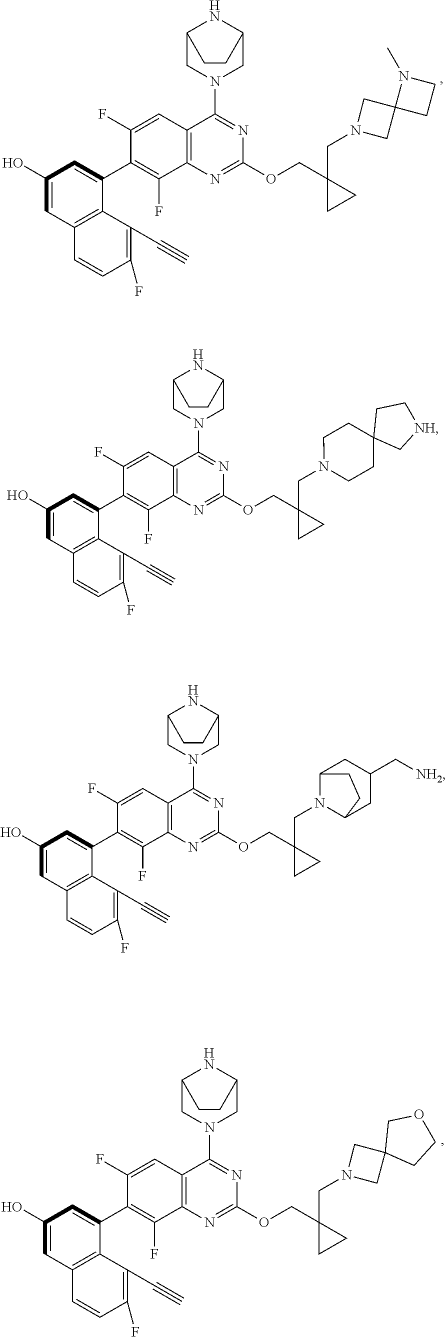

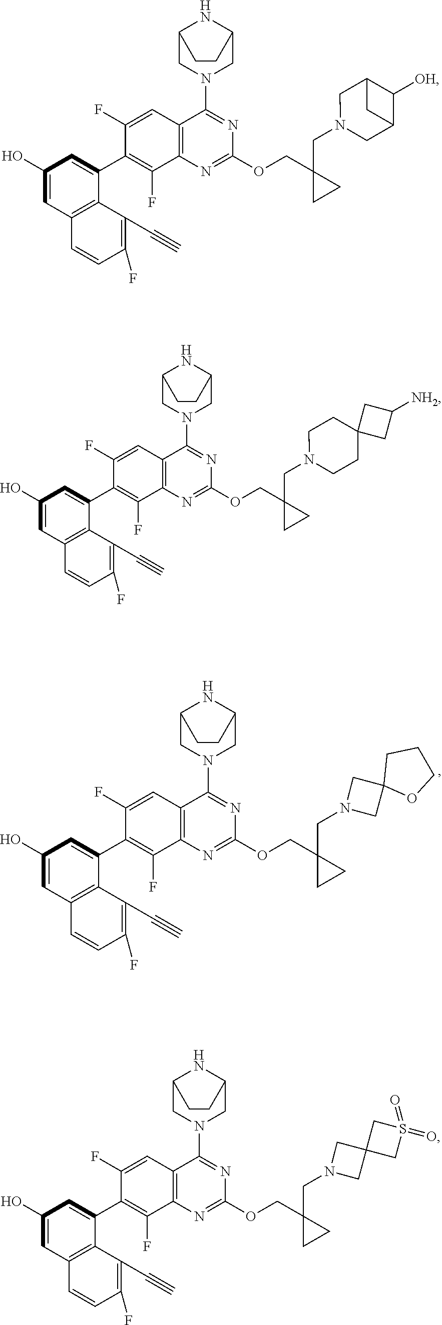

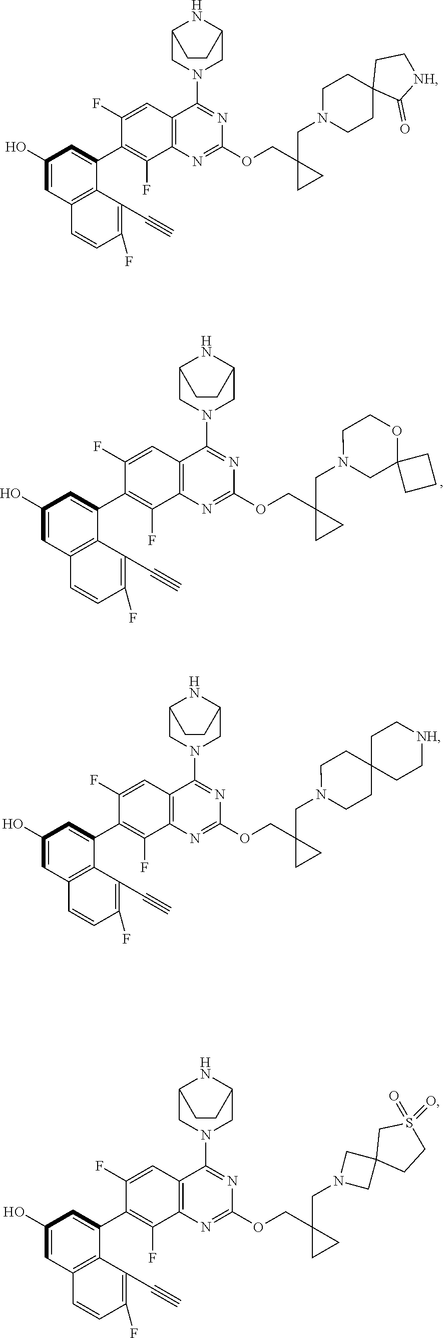

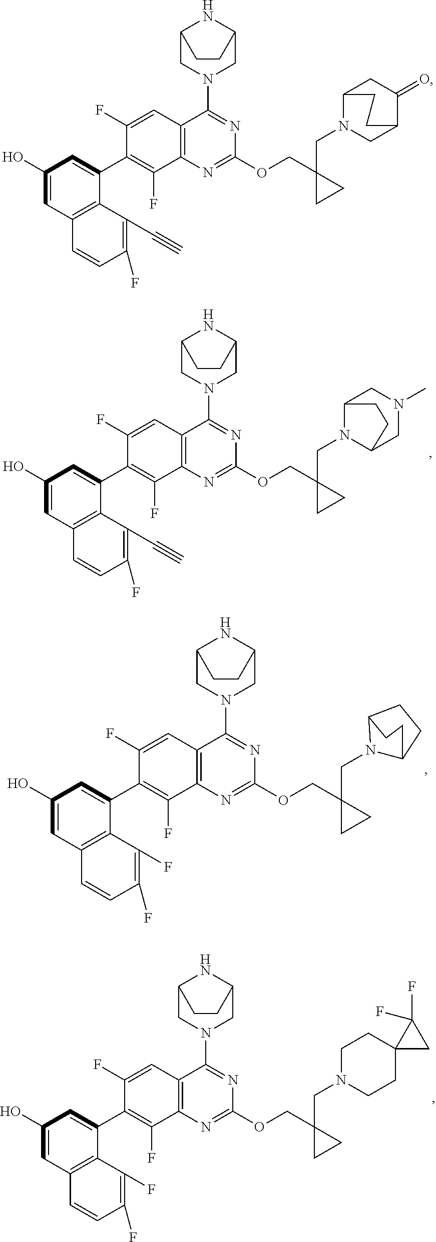

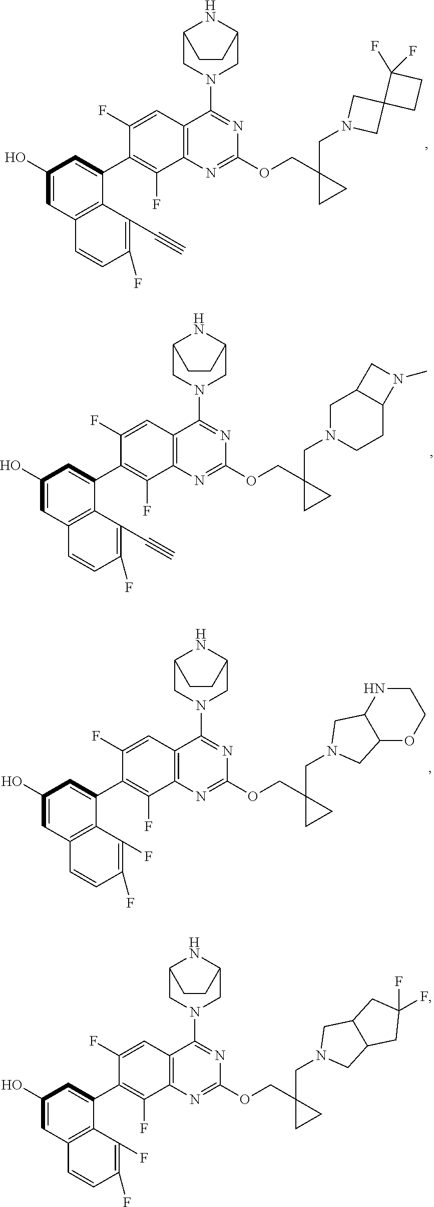

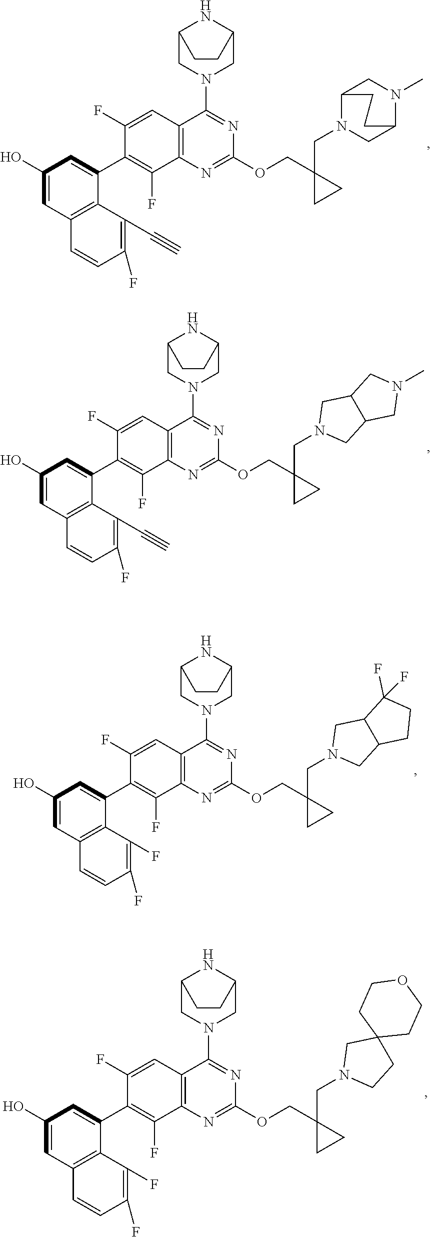

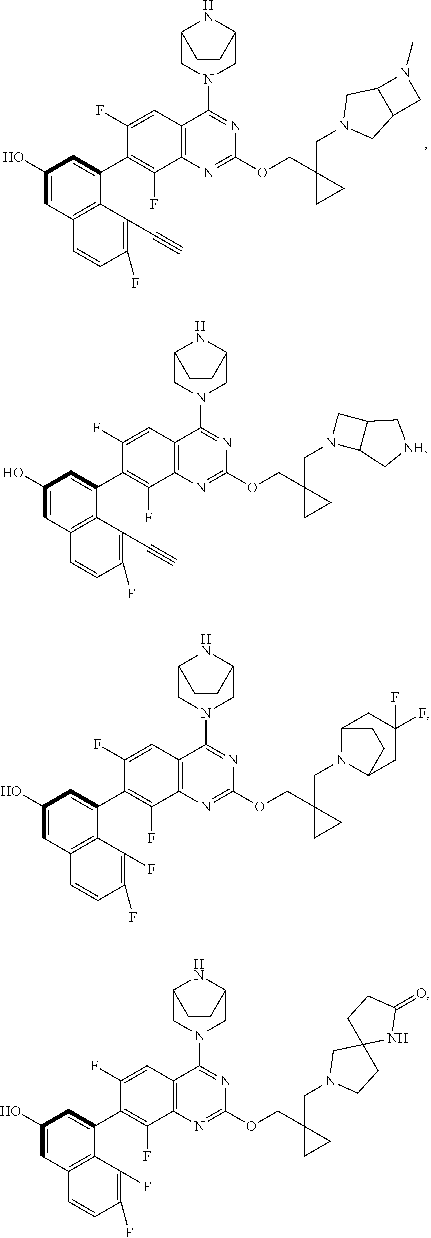

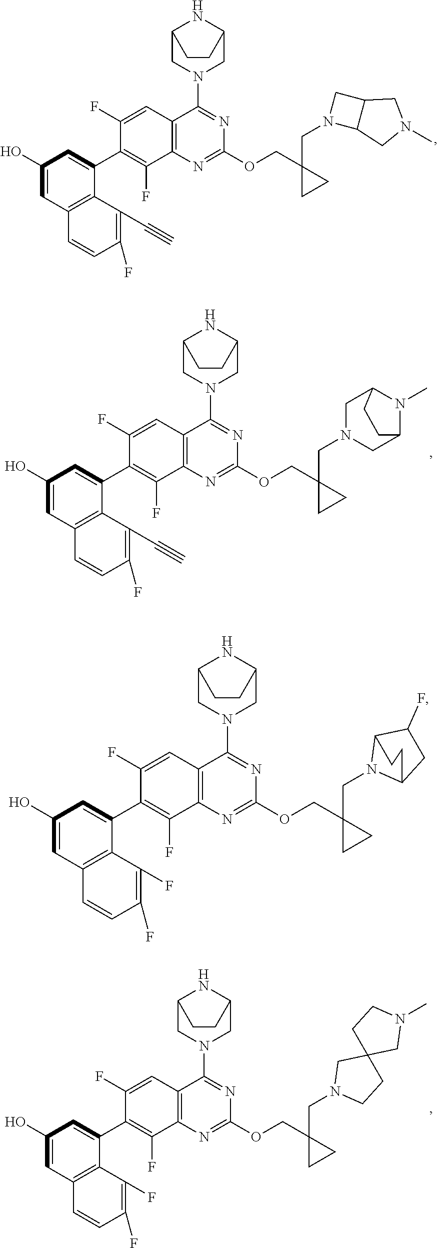

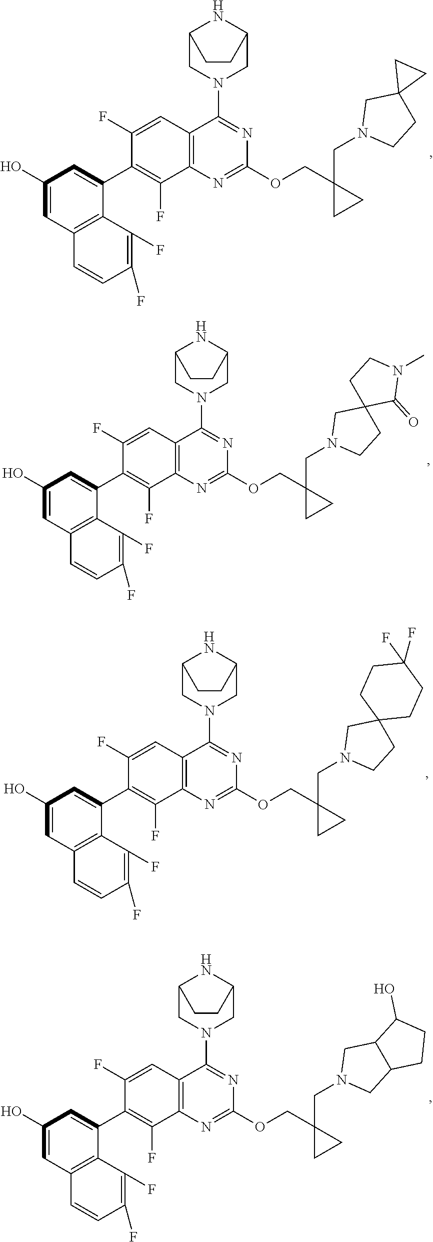

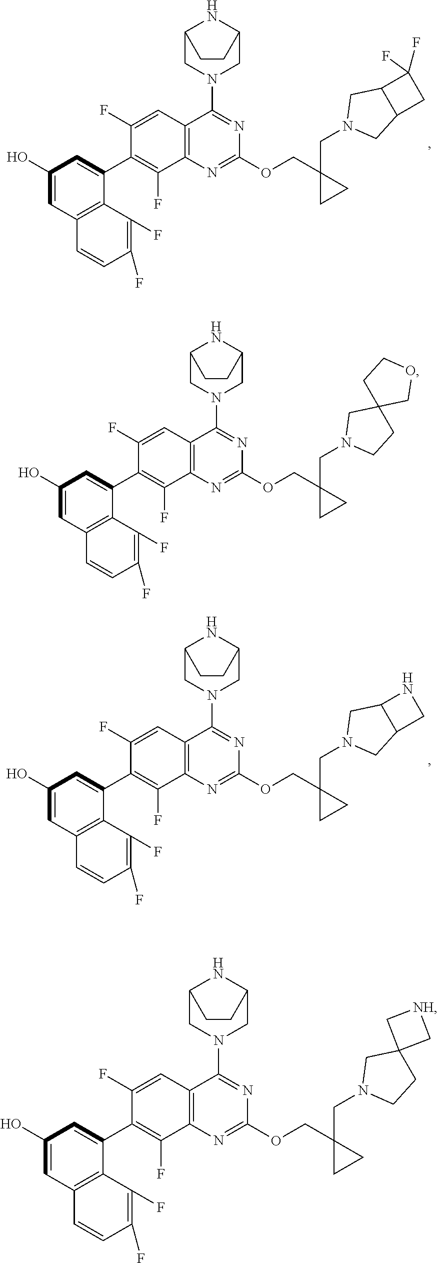

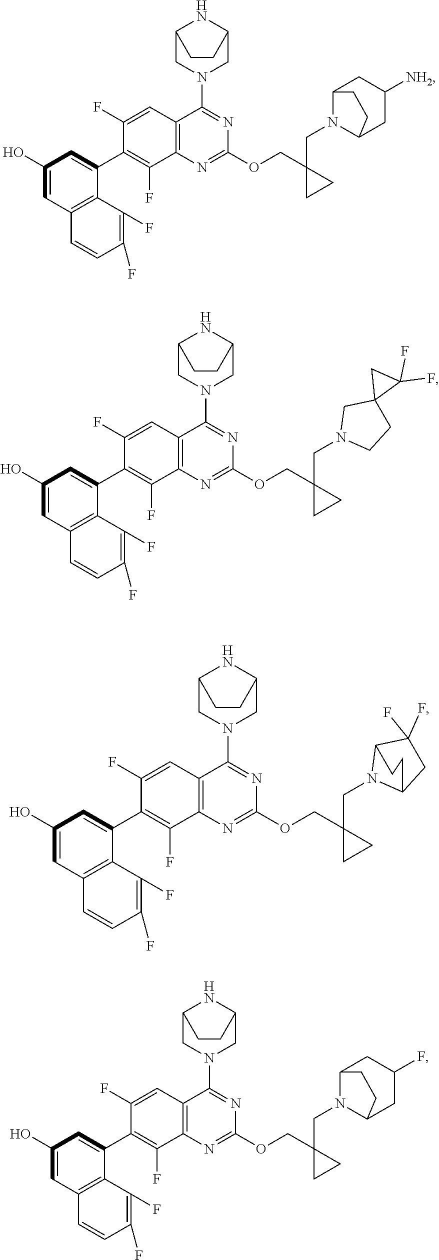

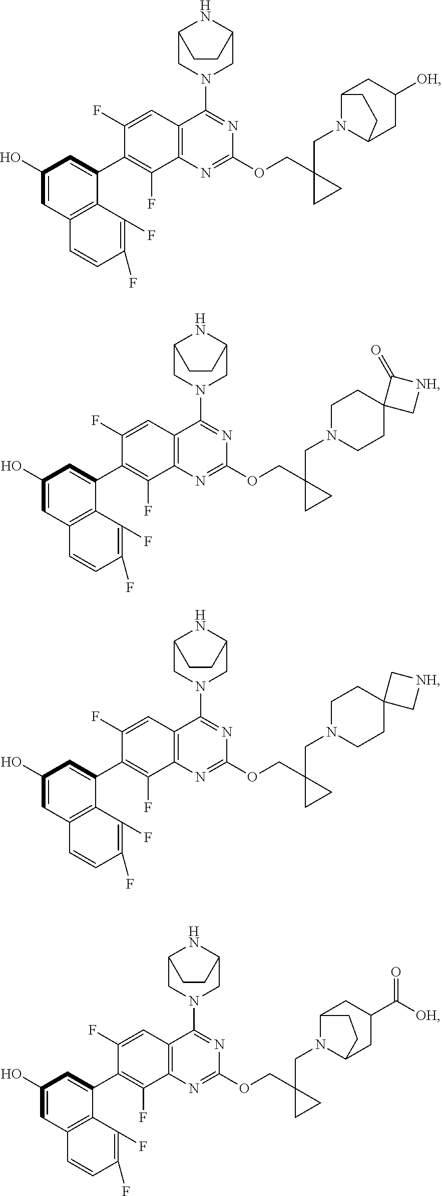

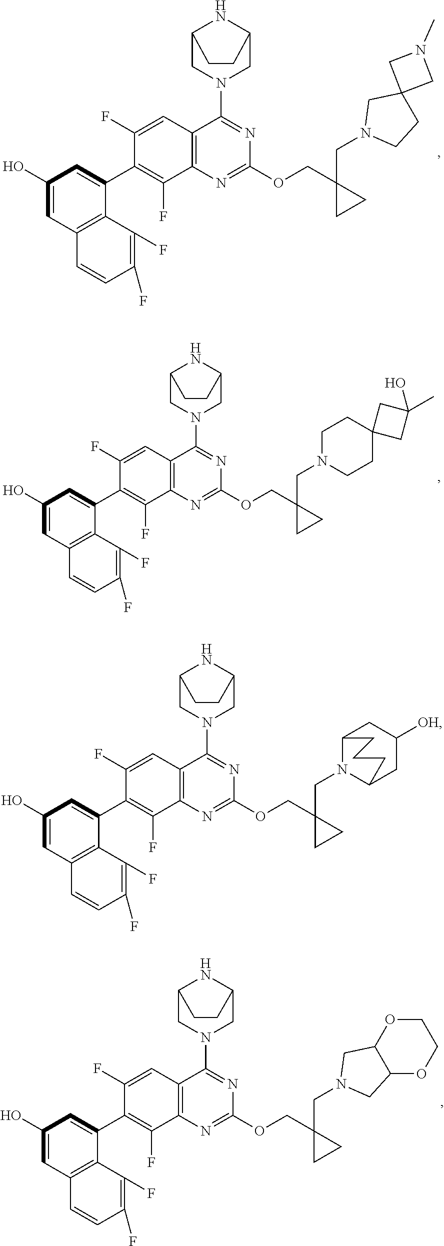

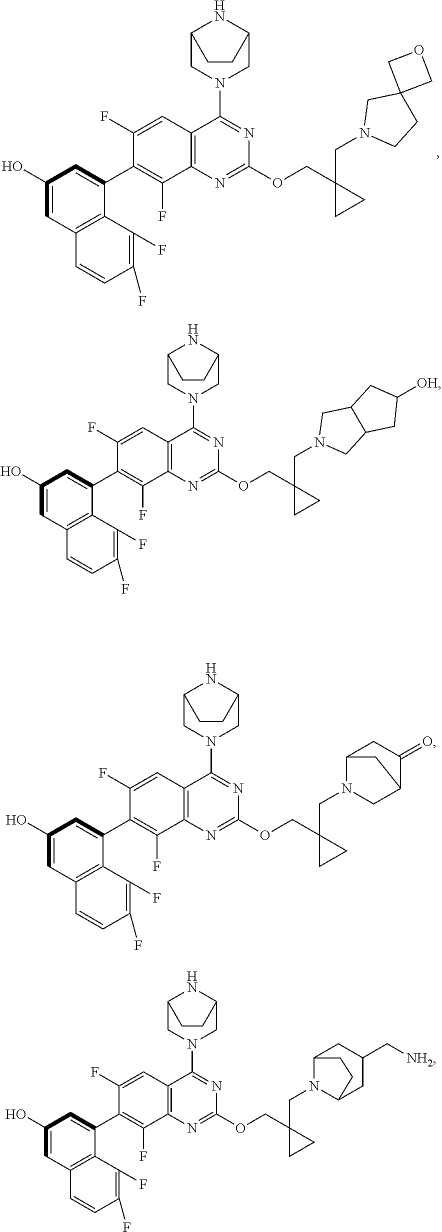

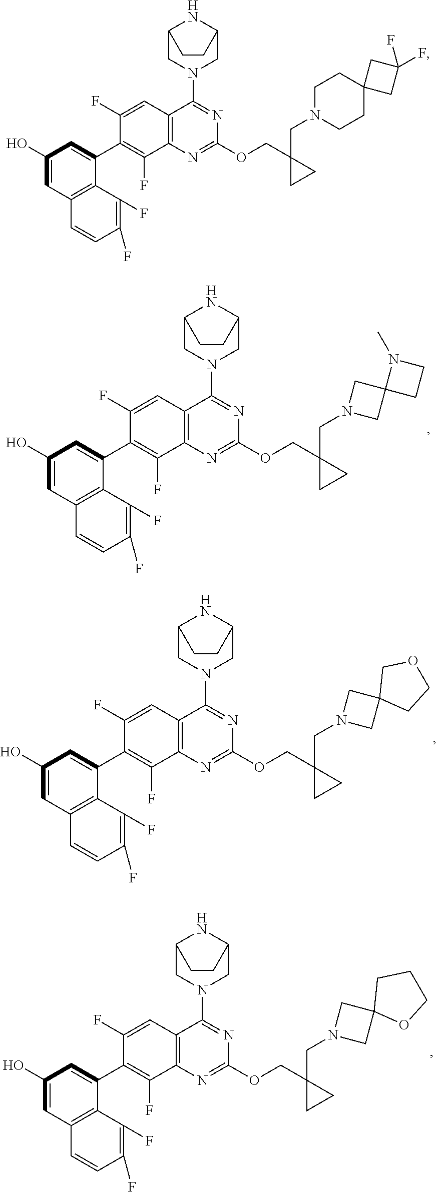

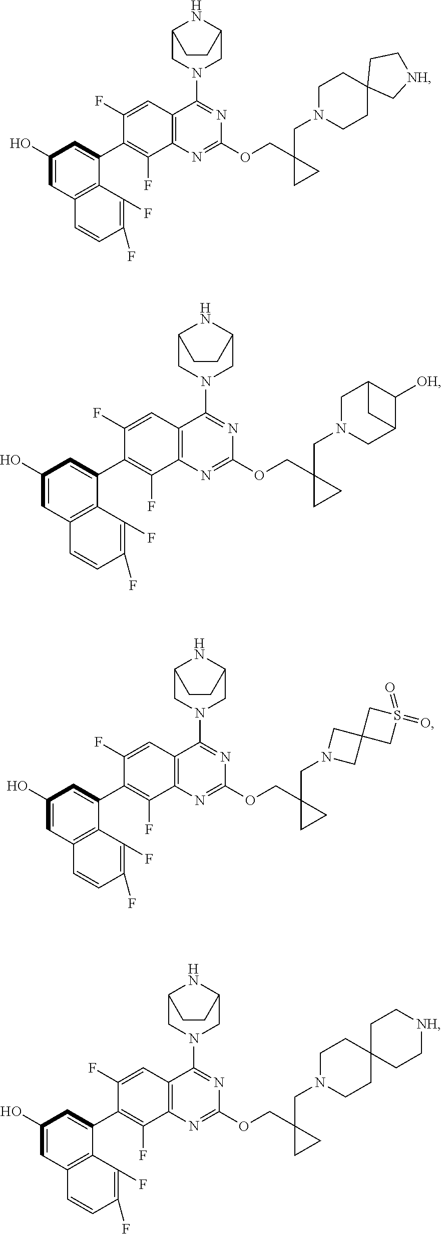

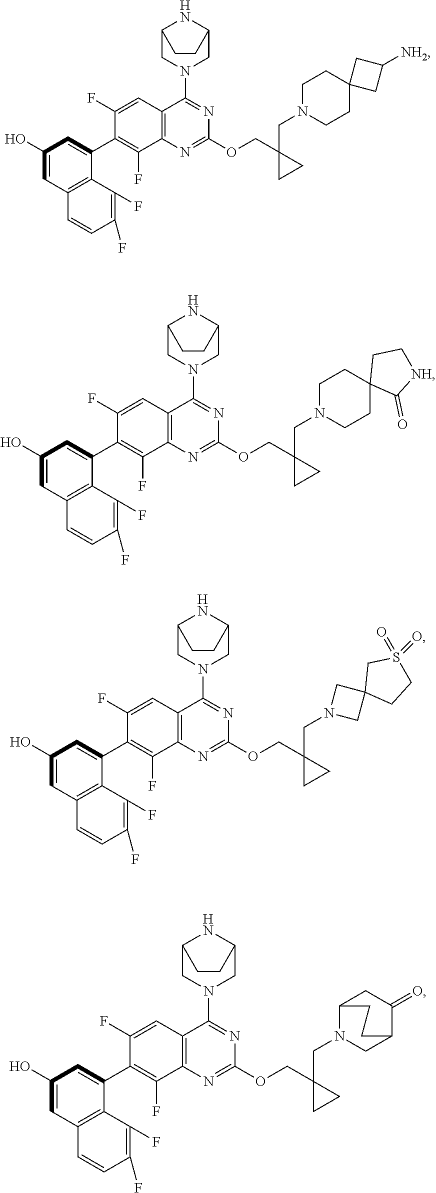

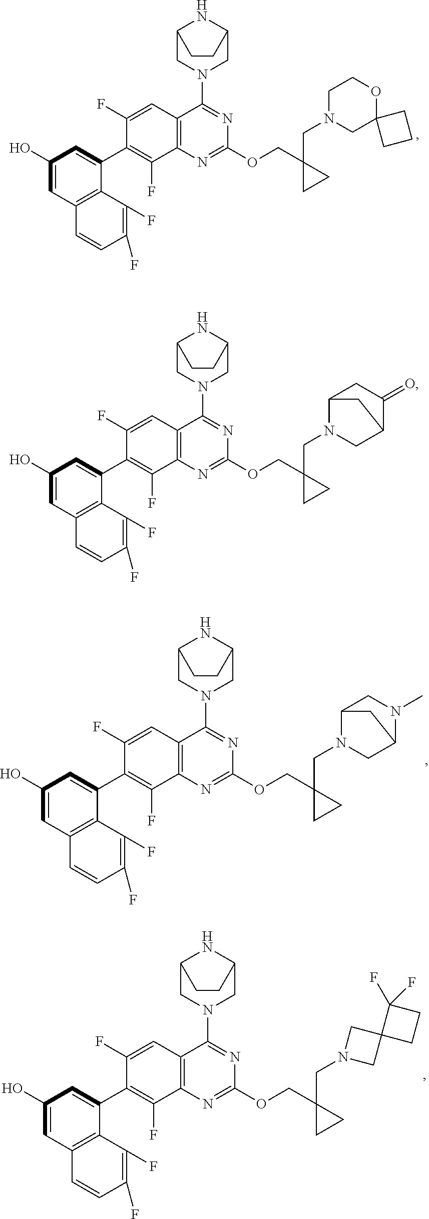

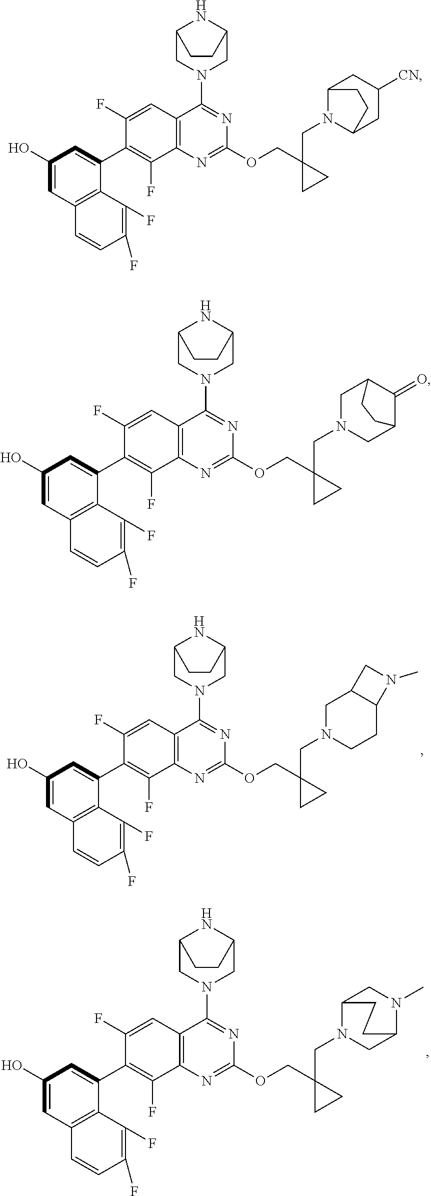

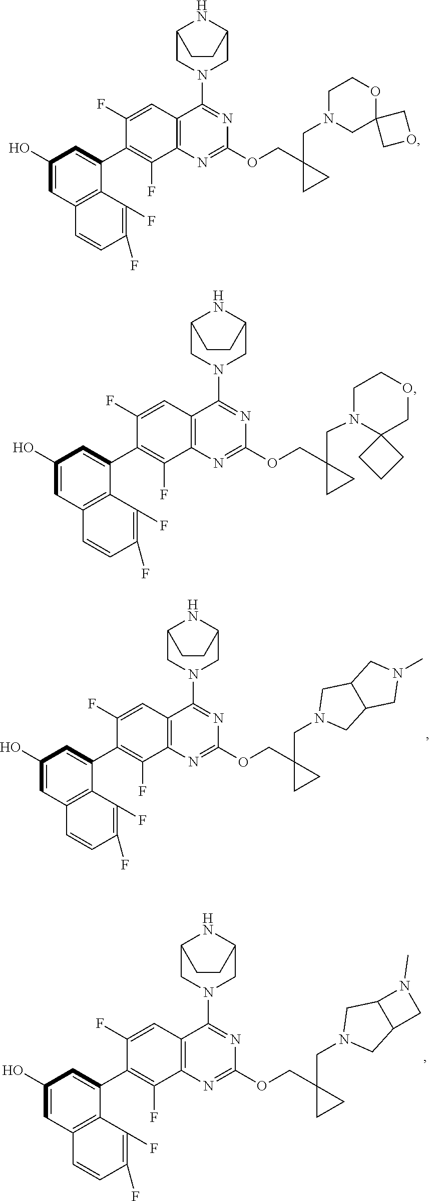

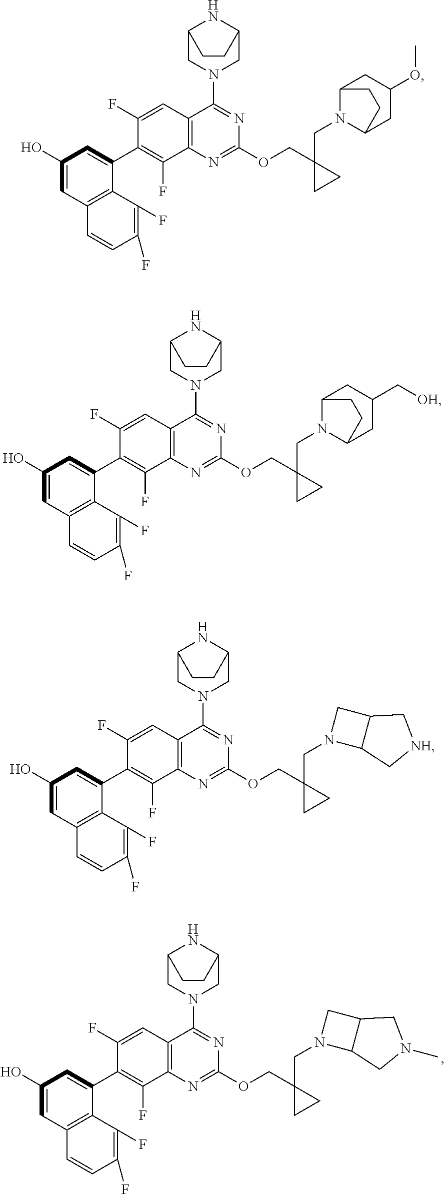

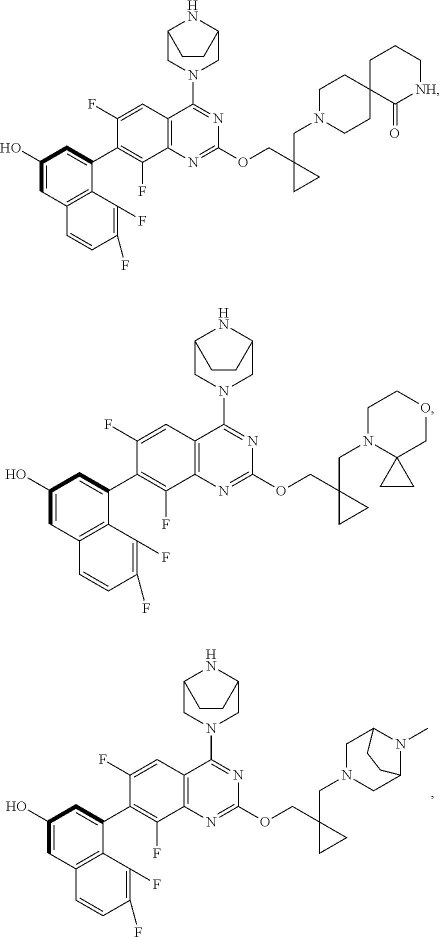

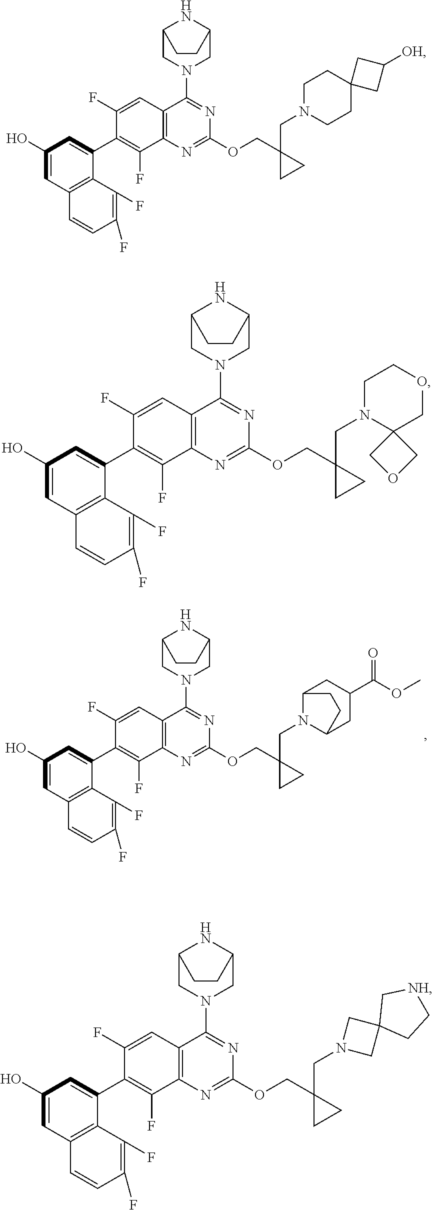

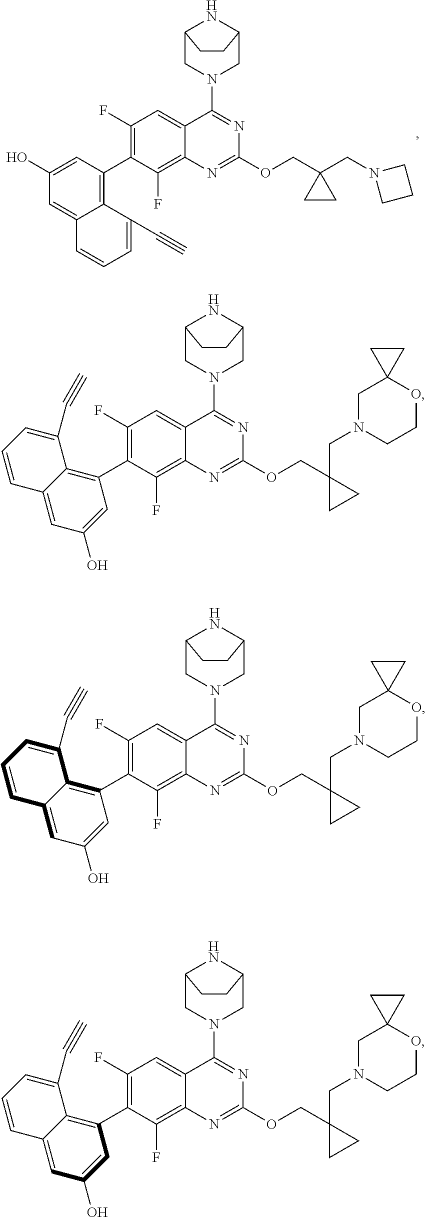

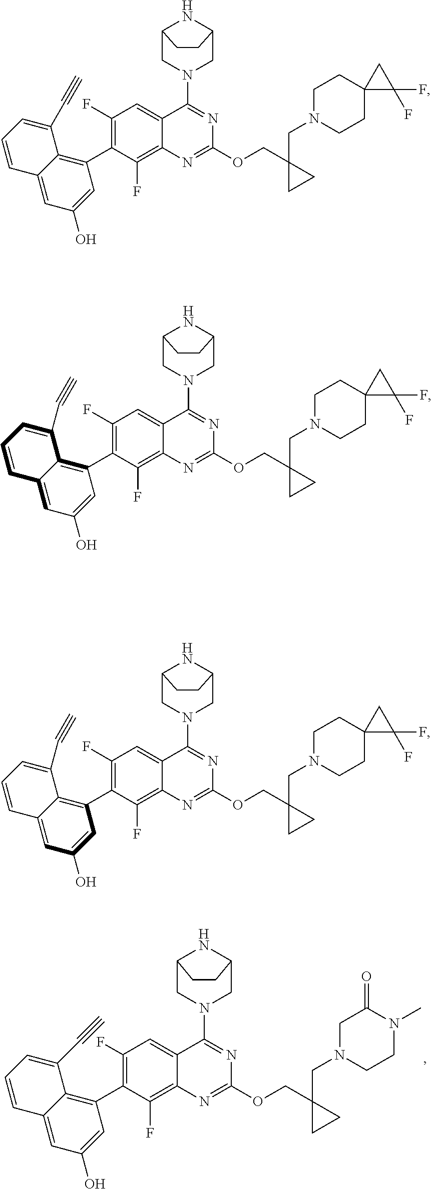

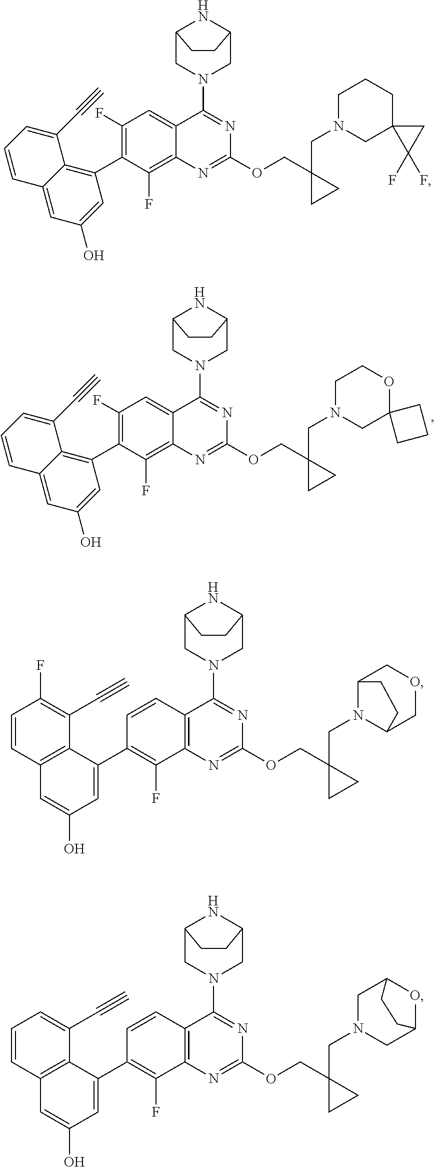 [SMILES: C#Cc1cccc2cc(O)cc(-c3c(F)cc4c(N5CC6CCC(C5)N6)nc(OCC5(CN6CCCC7(C6)CC7(F)F)CC5)nc4c3F)c12 ; C#Cc1cccc2cc(O)cc(-c3c(F)cc4c(N5CC6CCC(C5)N6)nc(OCC5(CN6CCOC7(CCC7)C6)CC5)nc4c3F)c12 ; C#Cc1c(F)ccc2cc(O)cc(-c3ccc4c(N5CC6CCC(C5)N6)nc(OCC5(CN6C7CCC6COC7)CC5)nc4c3F)c12 ; C#Cc1cccc2cc(O)cc(-c3ccc4c(N5CC6CCC(C5)N6)nc(OCC5(CN6CC7CCC(C6)O7)CC5)nc4c3F)c12]

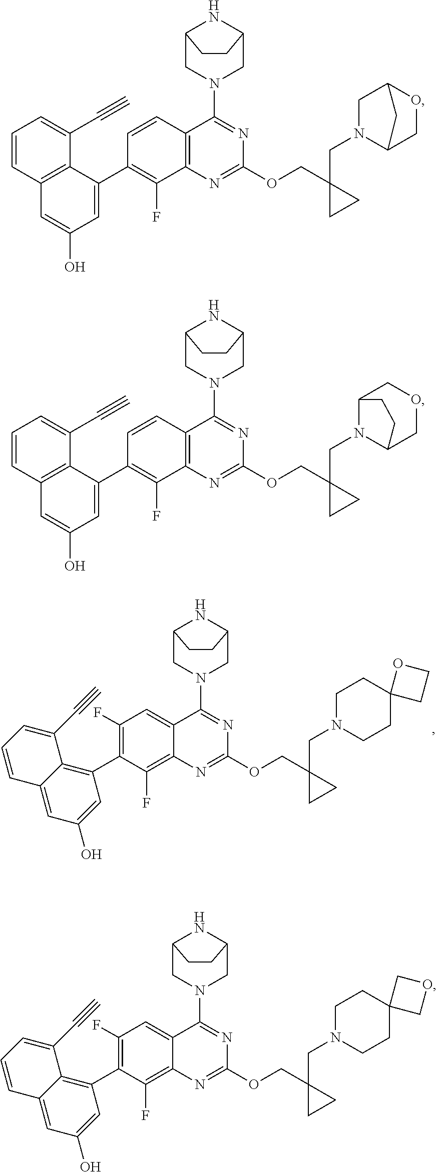 [SMILES: C#Cc1cccc2cc(O)cc(-c3ccc4c(N5CC6CCC(C5)N6)nc(OCC5(CN6CC7CC6CO7)CC5)nc4c3F)c12 ; C#Cc1cccc2cc(O)cc(-c3ccc4c(N5CC6CCC(C5)N6)nc(OCC5(CN6C7CCC6COC7)CC5)nc4c3F)c12 ; C#Cc1cccc2cc(O)cc(-c3c(F)cc4c(N5CC6CCC(C5)N6)nc(OCC5(CN6CCC7(CCO7)CC6)CC5)nc4c3F)c12 ; C#Cc1cccc2cc(O)cc(-c3c(F)cc4c(N5CC6CCC(C5)N6)nc(OCC5(CN6CCC7(CC6)COC7)CC5)nc4c3F)c12]

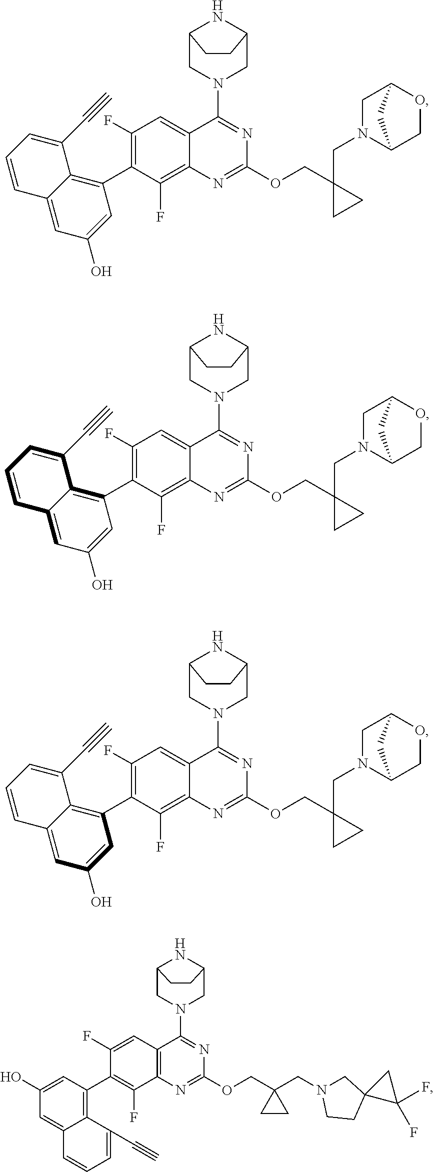

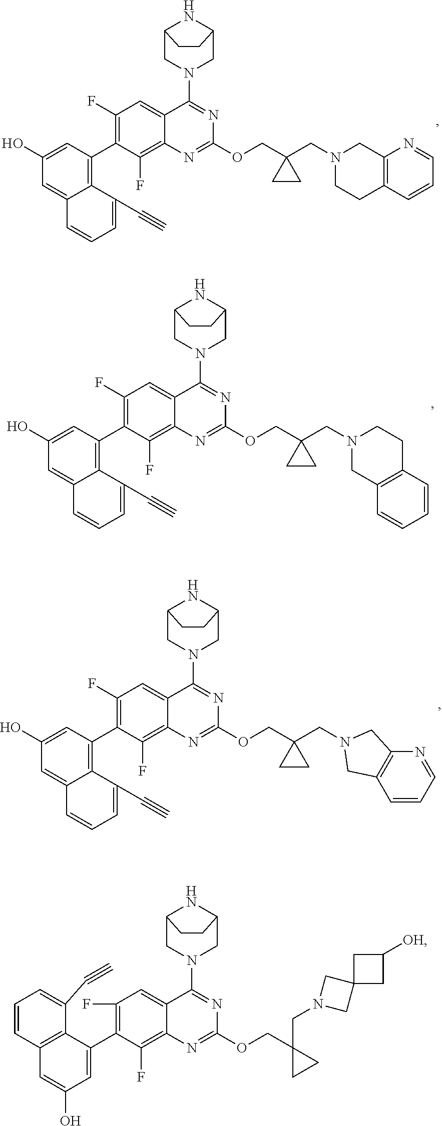 [SMILES: C#Cc1cccc2cc(O)cc(-c3c(F)cc4c(N5CC6CCC(C5)N6)nc(OCC5(CN6CCc7cccnc7C6)CC5)nc4c3F)c12 ; C#Cc1cccc2cc(O)cc(-c3c(F)cc4c(N5CC6CCC(C5)N6)nc(OCC5(CN6CCc7ccccc7C6)CC5)nc4c3F)c12 ; C#Cc1cccc2cc(O)cc(-c3c(F)cc4c(N5CC6CCC(C5)N6)nc(OCC5(CN6Cc7cccnc7C6)CC5)nc4c3F)c12 ; C#Cc1cccc2cc(O)cc(-c3c(F)cc4c(N5CC6CCC(C5)N6)nc(OCC5(CN6CC7(CC(O)C7)C6)CC5)nc4c3F)c12]

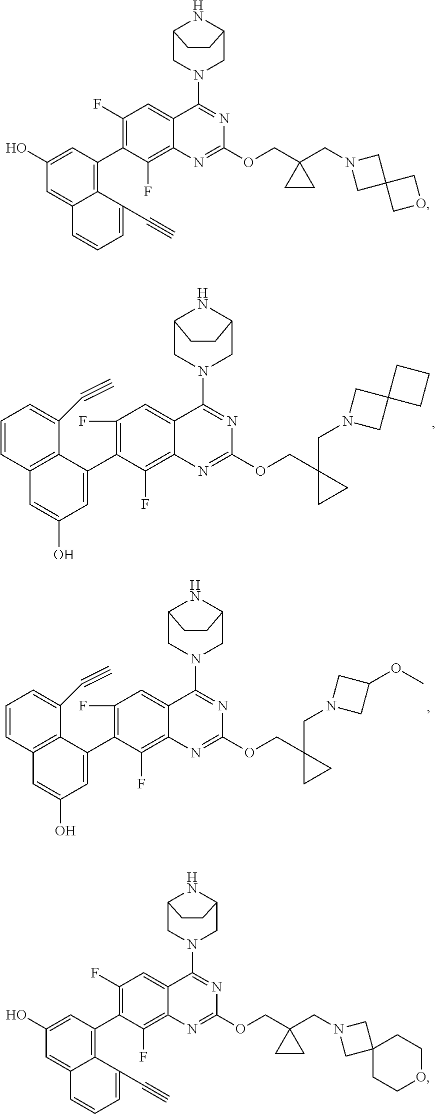 [SMILES: C#Cc1cccc2cc(O)cc(-c3c(F)cc4c(N5CC6CCC(C5)N6)nc(OCC5(CN6CC7(COC7)C6)CC5)nc4c3F)c12 ; C#Cc1cccc2cc(O)cc(-c3c(F)cc4c(N5CC6CCC(C5)N6)nc(OCC5(CN6CC7(CCC7)C6)CC5)nc4c3F)c12 ; C#Cc1cccc2cc(O)cc(-c3c(F)cc4c(N5CC6CCC(C5)N6)nc(OCC5(CN6CC(OC)C6)CC5)nc4c3F)c12 ; C#Cc1cccc2cc(O)cc(-c3c(F)cc4c(N5CC6CCC(C5)N6)nc(OCC5(CN6CC7(CCOCC7)C6)CC5)nc4c3F)c12]

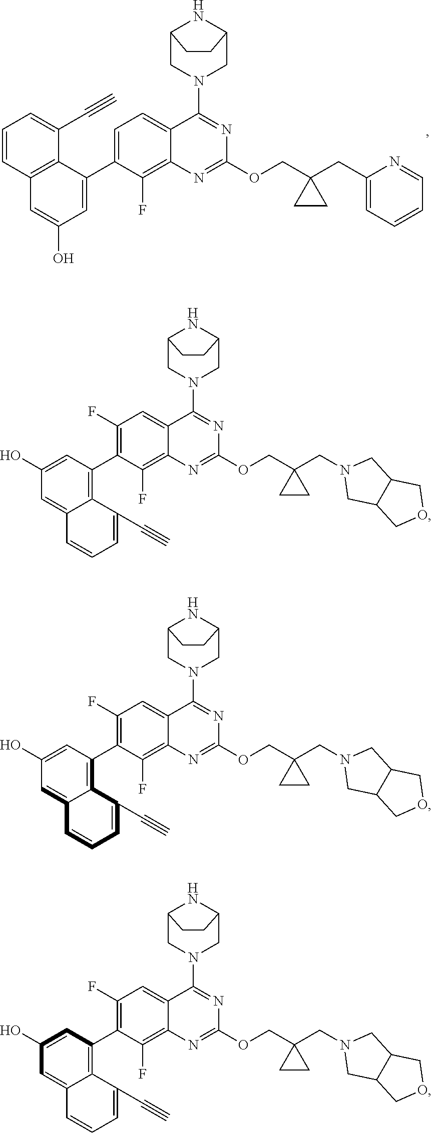

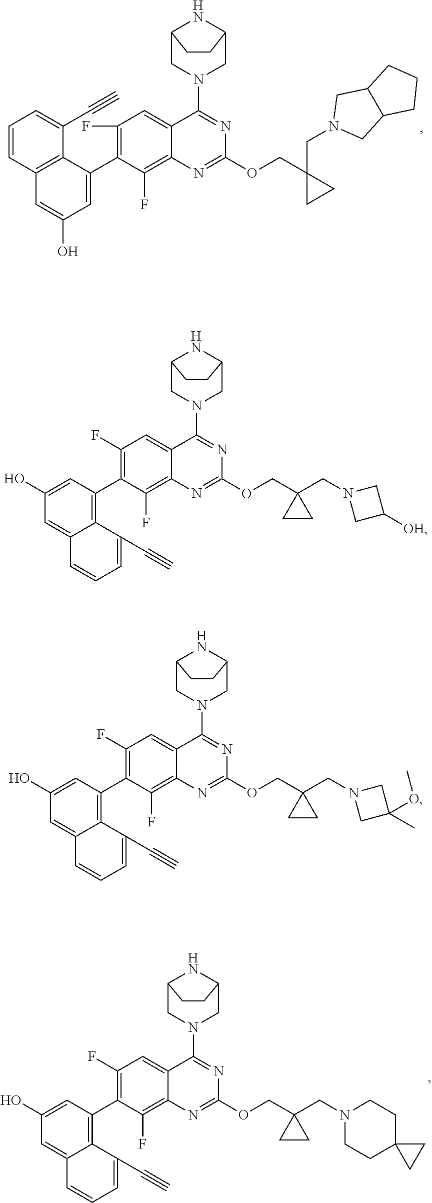 [SMILES: C#Cc1cccc2cc(O)cc(-c3c(F)cc4c(N5CC6CCC(C5)N6)nc(OCC5(CN6CC7CCCC7C6)CC5)nc4c3F)c12 ; C#Cc1cccc2cc(O)cc(-c3c(F)cc4c(N5CC6CCC(C5)N6)nc(OCC5(CN6CC(O)C6)CC5)nc4c3F)c12 ; C#Cc1cccc2cc(O)cc(-c3c(F)cc4c(N5CC6CCC(C5)N6)nc(OCC5(CN6CC(C)(OC)C6)CC5)nc4c3F)c12 ; C#Cc1cccc2cc(O)cc(-c3c(F)cc4c(N5CC6CCC(C5)N6)nc(OCC5(CN6CCC7(CC6)CC7)CC5)nc4c3F)c12]

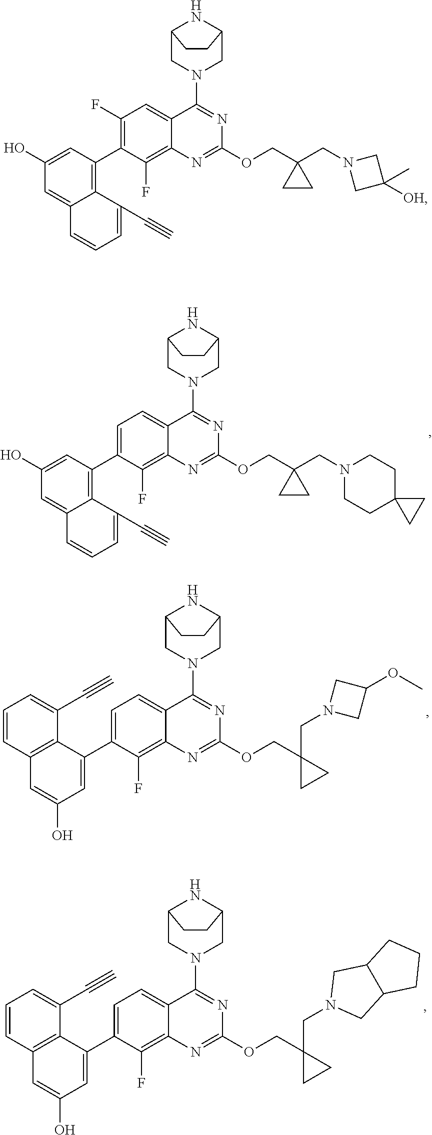 [SMILES: C#Cc1cccc2cc(O)cc(-c3c(F)cc4c(N5CC6CCC(C5)N6)nc(OCC5(CN6CC(C)(O)C6)CC5)nc4c3F)c12 ; C#Cc1cccc2cc(O)cc(-c3ccc4c(N5CC6CCC(C5)N6)nc(OCC5(CN6CCC7(CC6)CC7)CC5)nc4c3F)c12 ; C#Cc1cccc2cc(O)cc(-c3ccc4c(N5CC6CCC(C5)N6)nc(OCC5(CN6CC(OC)C6)CC5)nc4c3F)c12 ; C#Cc1cccc2cc(O)cc(-c3ccc4c(N5CC6CCC(C5)N6)nc(OCC5(CN6CC7CCCC7C6)CC5)nc4c3F)c12]

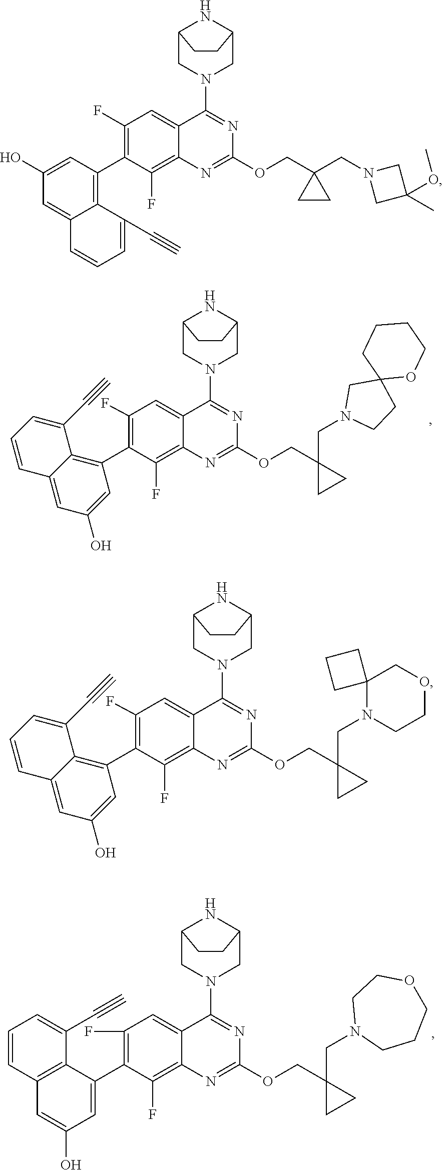 [SMILES: C#Cc1cccc2cc(O)cc(-c3c(F)cc4c(N5CC6CCC(C5)N6)nc(OCC5(CN6CC(C)(OC)C6)CC5)nc4c3F)c12 ; C#Cc1cccc2cc(O)cc(-c3c(F)cc4c(N5CC6CCC(C5)N6)nc(OCC5(CN6CCC7(CCCCO7)C6)CC5)nc4c3F)c12 ; C#Cc1cccc2cc(O)cc(-c3c(F)cc4c(N5CC6CCC(C5)N6)nc(OCC5(CN6CCOCC67CCC7)CC5)nc4c3F)c12 ; C#Cc1cccc2cc(O)cc(-c3c(F)cc4c(N5CC6CCC(C5)N6)nc(OCC5(CN6CCCOCC6)CC5)nc4c3F)c12]

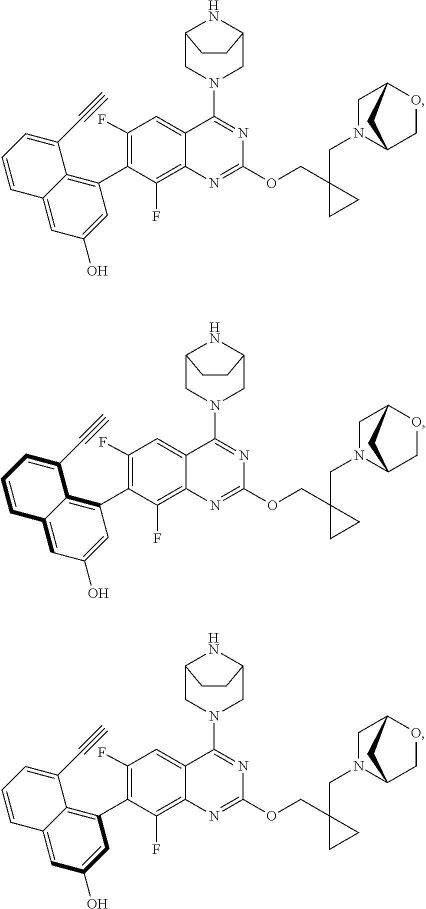

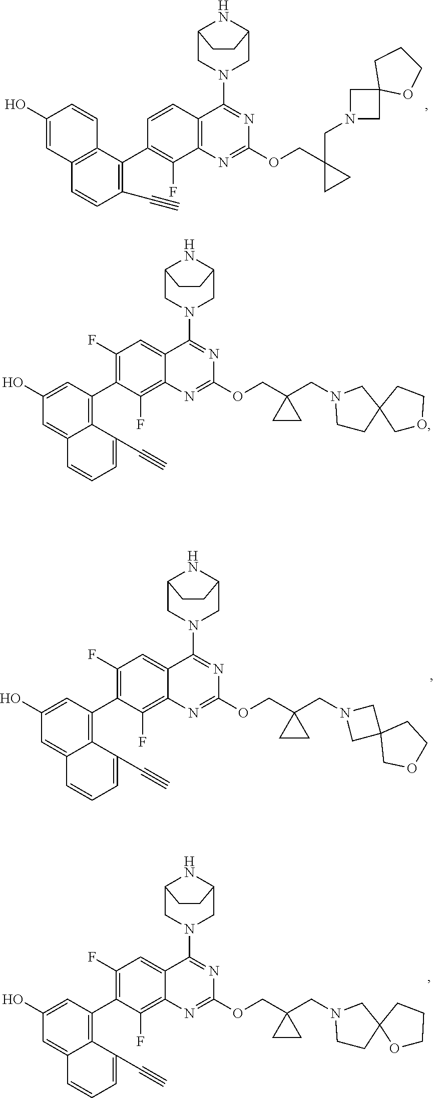 [SMILES: C#Cc1ccc2cc(O)ccc2c1-c1ccc2c(N3CC4CCC(C3)N4)nc(OCC3(CN4CC5(CCCO5)C4)CC3)nc2c1F ; C#Cc1cccc2cc(O)cc(-c3c(F)cc4c(N5CC6CCC(C5)N6)nc(OCC5(CN6CCC7(CCOC7)C6)CC5)nc4c3F)c12 ; C#Cc1cccc2cc(O)cc(-c3c(F)cc4c(N5CC6CCC(C5)N6)nc(OCC5(CN6CC7(CCOC7)C6)CC5)nc4c3F)c12 ; C#Cc1cccc2cc(O)cc(-c3c(F)cc4c(N5CC6CCC(C5)N6)nc(OCC5(CN6CCC7(CCCO7)C6)CC5)nc4c3F)c12]

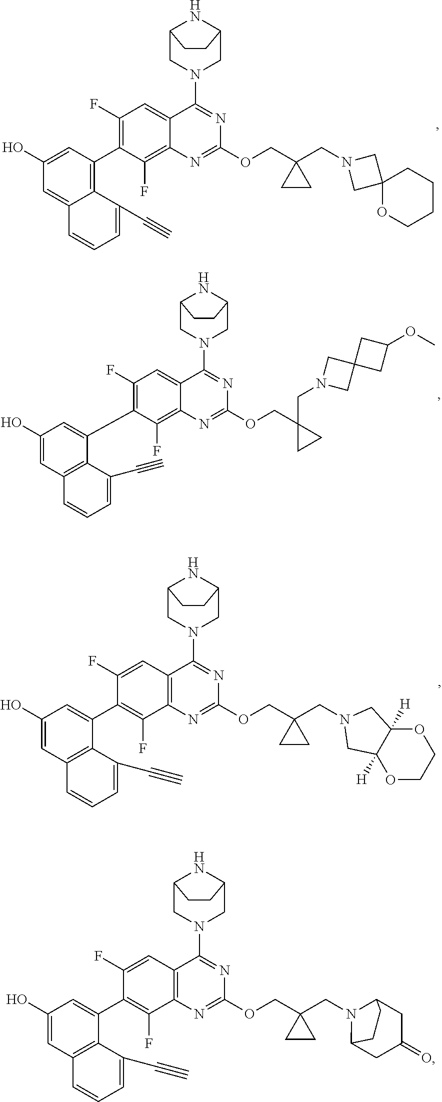 [SMILES: C#Cc1cccc2cc(O)cc(-c3c(F)cc4c(N5CC6CCC(C5)N6)nc(OCC5(CN6CC7(CCCCO7)C6)CC5)nc4c3F)c12 ; C#Cc1cccc2cc(O)cc(-c3c(F)cc4c(N5CC6CCC(C5)N6)nc(OCC5(CN6CC7(CC(OC)C7)C6)CC5)nc4c3F)c12 ; C#Cc1cccc2cc(O)cc(-c3c(F)cc4c(N5CC6CCC(C5)N6)nc(OCC5(CN6C[C@@H]7OCCO[C@@H]7C6)CC5)nc4c3F)c12 ; C#Cc1cccc2cc(O)cc(-c3c(F)cc4c(N5CC6CCC(C5)N6)nc(OCC5(CN6C7CCC6CC(=O)C7)CC5)nc4c3F)c12]

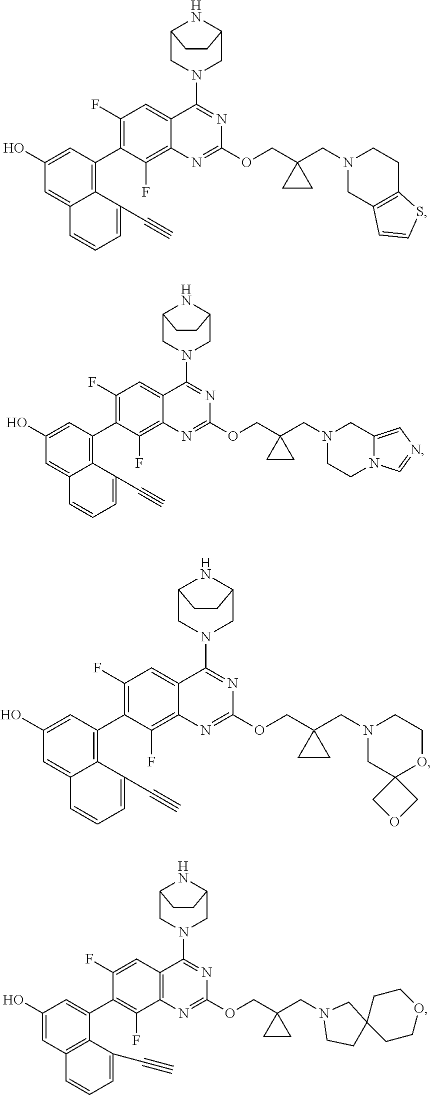 [SMILES: C#Cc1cccc2cc(O)cc(-c3c(F)cc4c(N5CC6CCC(C5)N6)nc(OCC5(CN6CCc7sccc7C6)CC5)nc4c3F)c12 ; C#Cc1cccc2cc(O)cc(-c3c(F)cc4c(N5CC6CCC(C5)N6)nc(OCC5(CN6CCn7cncc7C6)CC5)nc4c3F)c12 ; C#Cc1cccc2cc(O)cc(-c3c(F)cc4c(N5CC6CCC(C5)N6)nc(OCC5(CN6CCOC7(COC7)C6)CC5)nc4c3F)c12 ; C#Cc1cccc2cc(O)cc(-c3c(F)cc4c(N5CC6CCC(C5)N6)nc(OCC5(CN6CCC7(CCOCC7)C6)CC5)nc4c3F)c12]

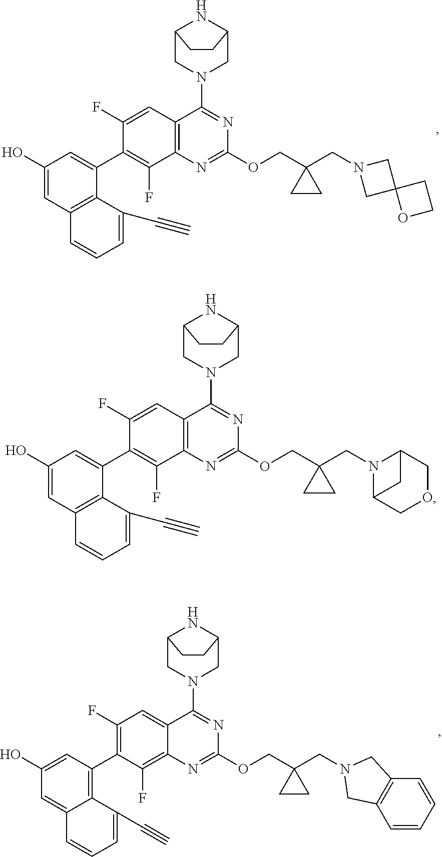 [SMILES: C#Cc1cccc2cc(O)cc(-c3c(F)cc4c(N5CC6CCC(C5)N6)nc(OCC5(CN6CC7(CCO7)C6)CC5)nc4c3F)c12 ; C#Cc1cccc2cc(O)cc(-c3c(F)cc4c(N5CC6CCC(C5)N6)nc(OCC5(CN6C7COCC6C7)CC5)nc4c3F)c12 ; C#Cc1cccc2cc(O)cc(-c3c(F)cc4c(N5CC6CCC(C5)N6)nc(OCC5(CN6Cc7ccccc7C6)CC5)nc4c3F)c12]

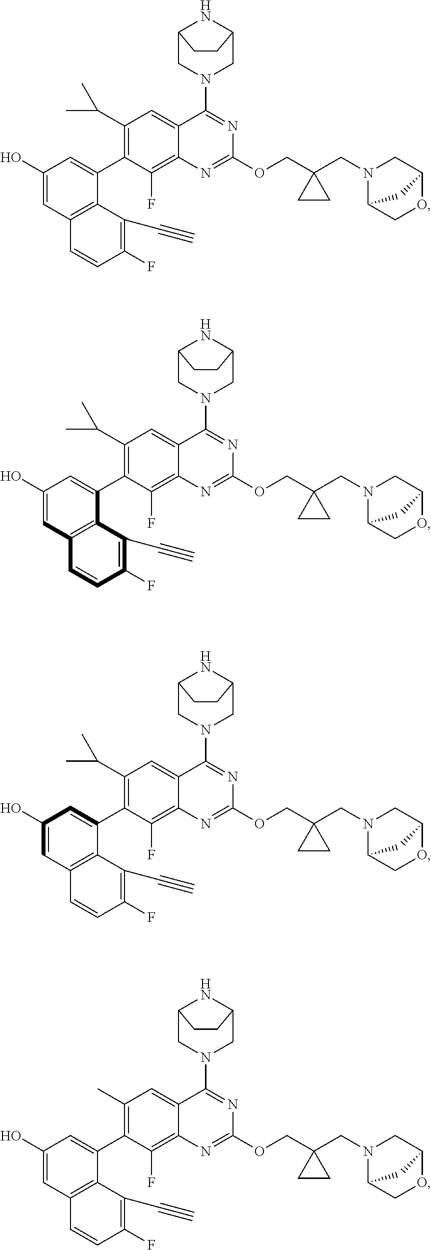

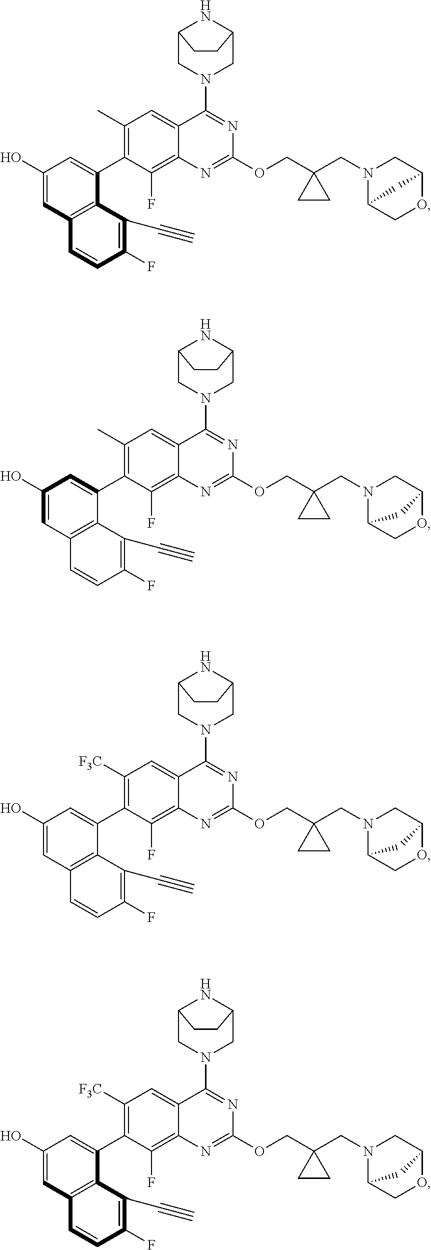

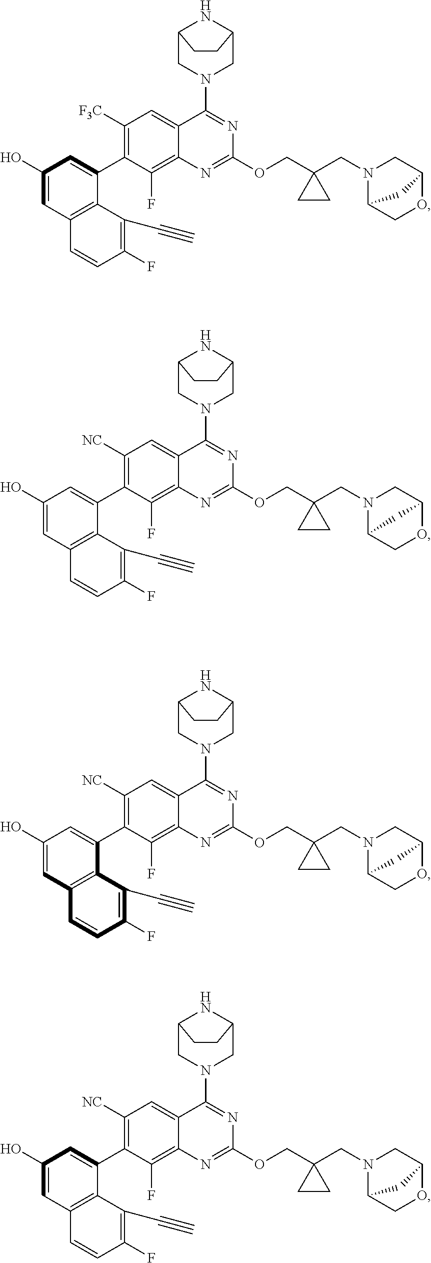

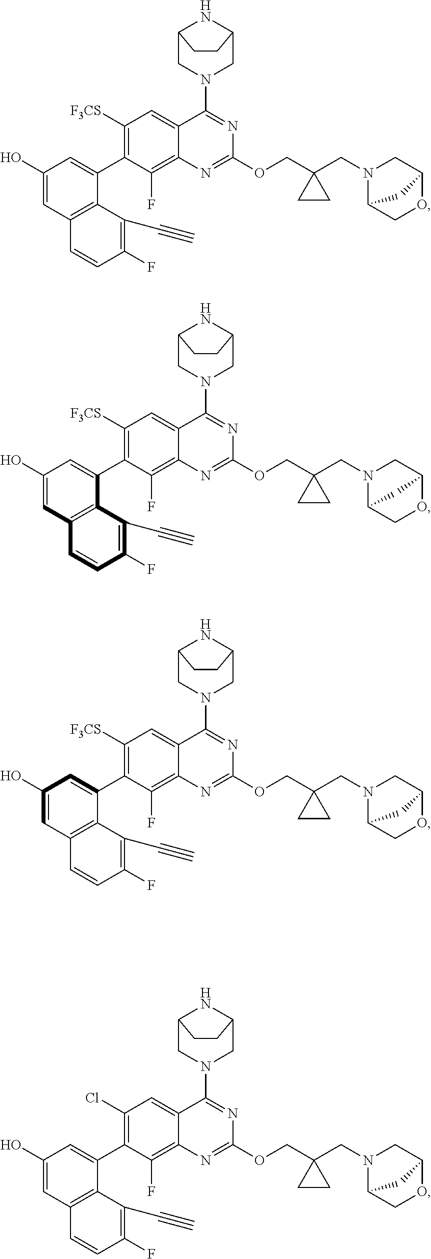

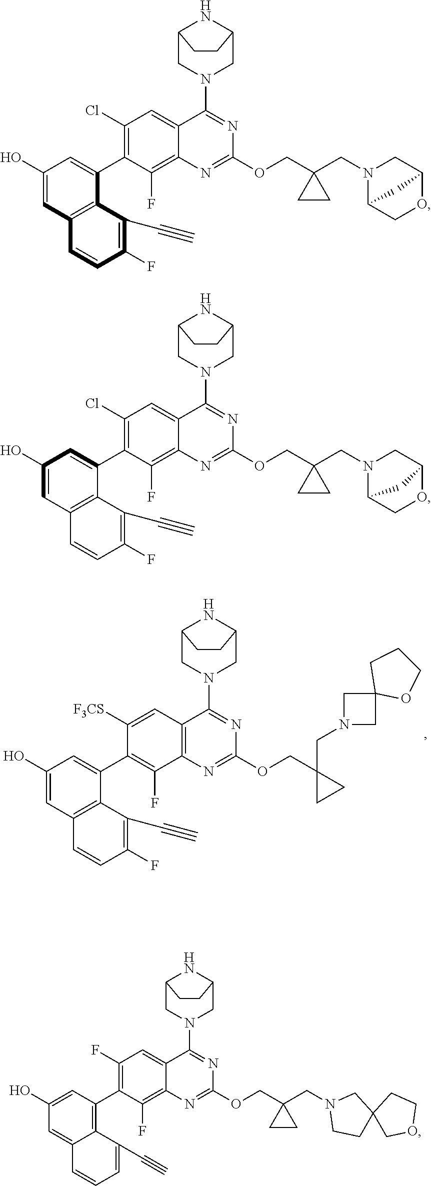

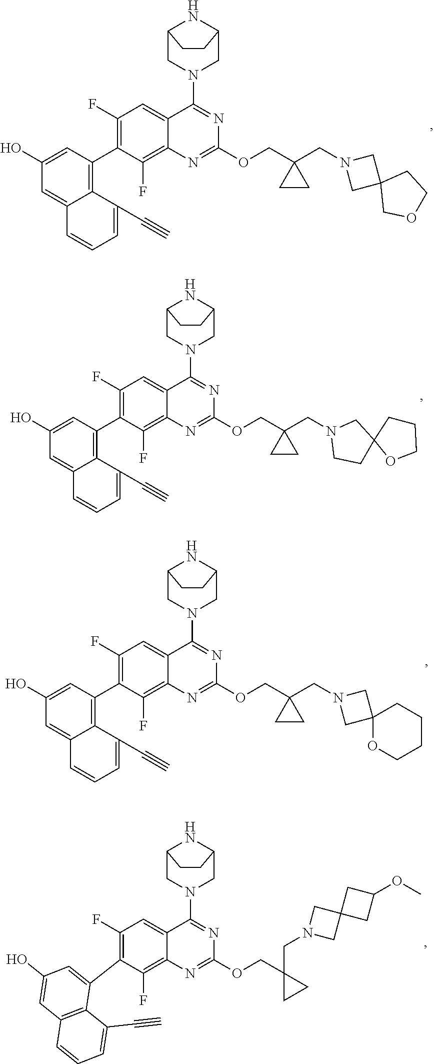 [SMILES: C#Cc1cccc2cc(O)cc(-c3c(F)cc4c(N5CC6CCC(C5)N6)nc(OCC5(CN6CC7(CCOC7)C6)CC5)nc4c3F)c12 ; C#Cc1cccc2cc(O)cc(-c3c(F)cc4c(N5CC6CCC(C5)N6)nc(OCC5(CN6CCC7(CCCO7)C6)CC5)nc4c3F)c12 ; C#Cc1cccc2cc(O)cc(-c3c(F)cc4c(N5CC6CCC(C5)N6)nc(OCC5(CN6CC7(CCCCO7)C6)CC5)nc4c3F)c12 ; C#Cc1cccc2cc(O)cc(-c3c(F)cc4c(N5CC6CCC(C5)N6)nc(OCC5(CN6CC7(CC(OC)C7)C6)CC5)nc4c3F)c12]

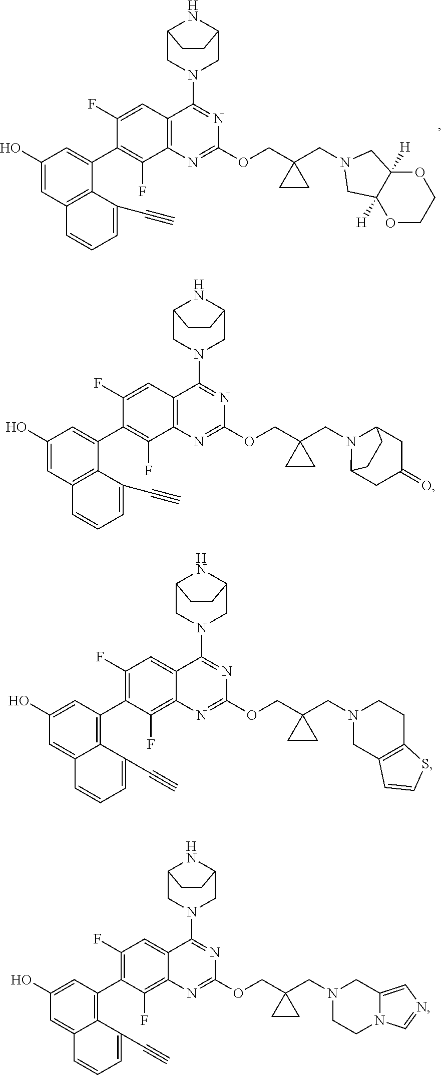 [SMILES: C#Cc1cccc2cc(O)cc(-c3c(F)cc4c(N5CC6CCC(C5)N6)nc(OCC5(CN6C[C@@H]7OCCO[C@@H]7C6)CC5)nc4c3F)c12 ; C#Cc1cccc2cc(O)cc(-c3c(F)cc4c(N5CC6CCC(C5)N6)nc(OCC5(CN6C7CCC6CC(=O)C7)CC5)nc4c3F)c12 ; C#Cc1cccc2cc(O)cc(-c3c(F)cc4c(N5CC6CCC(C5)N6)nc(OCC5(CN6CCc7sccc7C6)CC5)nc4c3F)c12 ; C#Cc1cccc2cc(O)cc(-c3c(F)cc4c(N5CC6CCC(C5)N6)nc(OCC5(CN6CCn7cncc7C6)CC5)nc4c3F)c12]

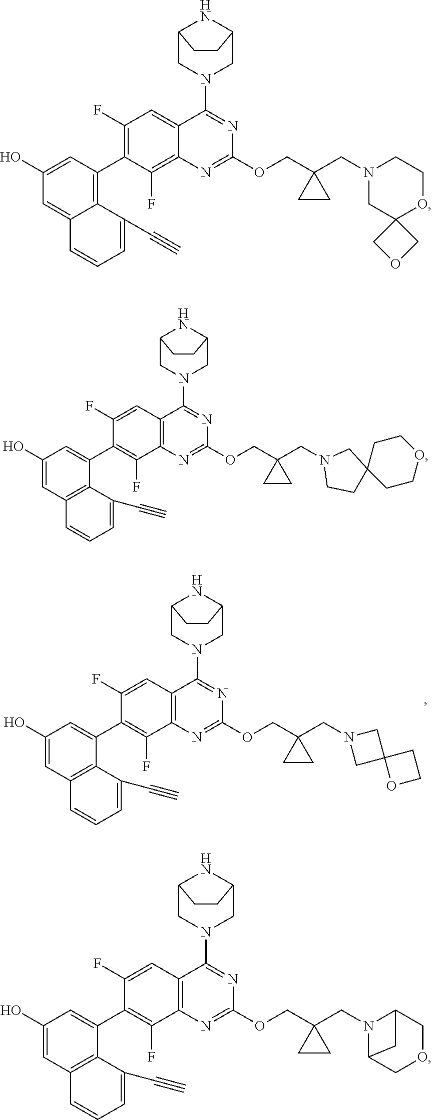 [SMILES: C#Cc1cccc2cc(O)cc(-c3c(F)cc4c(N5CC6CCC(C5)N6)nc(OCC5(CN6CCOC7(COC7)C6)CC5)nc4c3F)c12 ; C#Cc1cccc2cc(O)cc(-c3c(F)cc4c(N5CC6CCC(C5)N6)nc(OCC5(CN6CCC7(CCOCC7)C6)CC5)nc4c3F)c12 ; C#Cc1cccc2cc(O)cc(-c3c(F)cc4c(N5CC6CCC(C5)N6)nc(OCC5(CN6CC7(CCO7)C6)CC5)nc4c3F)c12 ; C#Cc1cccc2cc(O)cc(-c3c(F)cc4c(N5CC6CCC(C5)N6)nc(OCC5(CN6C7COCC6C7)CC5)nc4c3F)c12]

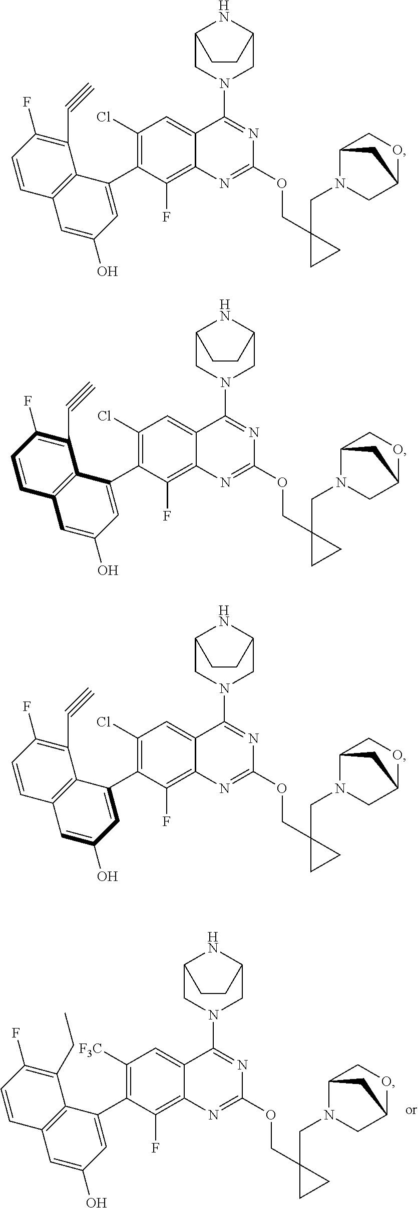

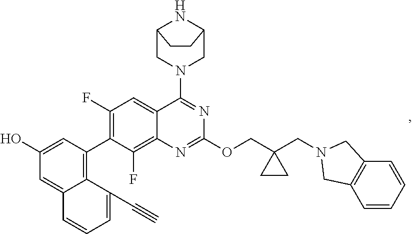 [SMILES: C#Cc1cccc2cc(O)cc(-c3c(F)cc4c(N5CC6CCC(C5)N6)nc(OCC5(CN6Cc7ccccc7C6)CC5)nc4c3F)c12]

[105]. The compound according to any one of [1] to [104], wherein, the compound is selected from any one of the following compounds:

An atropisomer, which is afforded by chiral separation of the compound having the structure

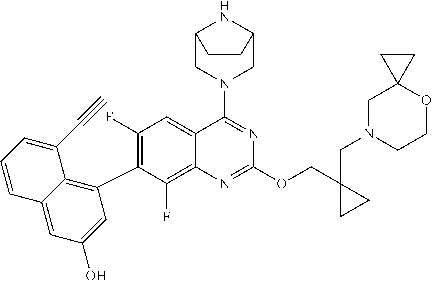 [SMILES: C#Cc1cccc2cc(O)cc(-c3c(F)cc4c(N5CC6CCC(C5)N6)nc(OCC5(CN6CCOC7(CC7)C6)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.266 min;

An atropisomer, which is afforded by chiral separation of the compound having the structure

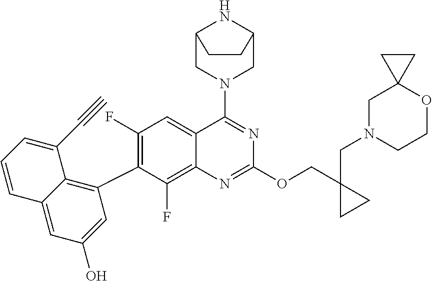 [SMILES: C#Cc1cccc2cc(O)cc(-c3c(F)cc4c(N5CC6CCC(C5)N6)nc(OCC5(CN6CCOC7(CC7)C6)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 6.685 min;

An atropisomer, which is afforded by chiral separation of the compound having the structure

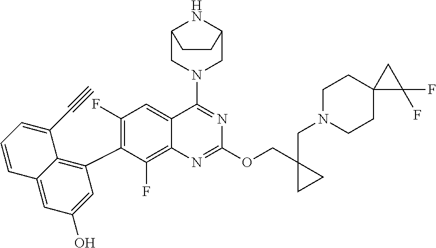 [SMILES: C#Cc1cccc2cc(O)cc(-c3c(F)cc4c(N5CC6CCC(C5)N6)nc(OCC5(CN6CCC7(CC6)CC7(F)F)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 3.665 min;

An atropisomer, which is afforded by chiral separation of the compound having the structure

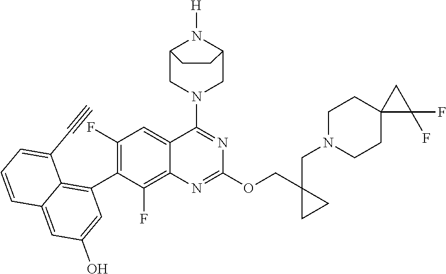 [SMILES: C#Cc1cccc2cc(O)cc(-c3c(F)cc4c(N5CC6CCC(C5)N6)nc(OCC5(CN6CCC7(CC6)CC7(F)F)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 5.532 min;

An atropisomer, which is afforded by chiral separation of the compound having the structure

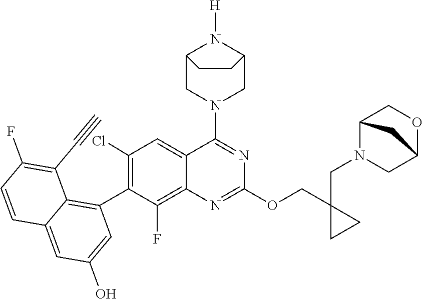 [SMILES: C#Cc1c(F)ccc2cc(O)cc(-c3c(Cl)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.561 min;

An atropisomer, which is afforded by chiral separation of the compound having the structure

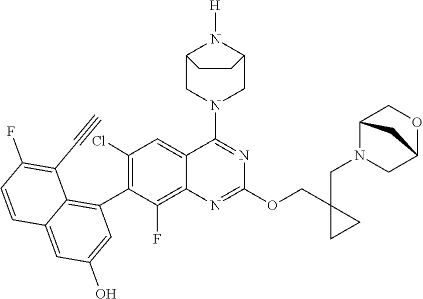 [SMILES: C#Cc1c(F)ccc2cc(O)cc(-c3c(Cl)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.002 min;

An atropisomer, which is afforded by chiral separation of the compound having the structure

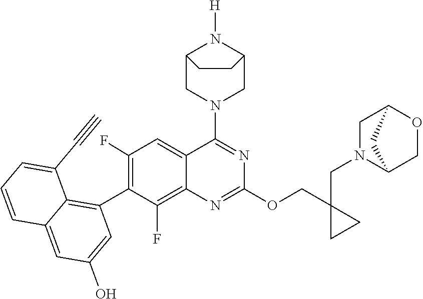 [SMILES: C#Cc1cccc2cc(O)cc(-c3c(F)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.297 min;

An atropisomer, which is afforded by chiral separation of the compound having the structure

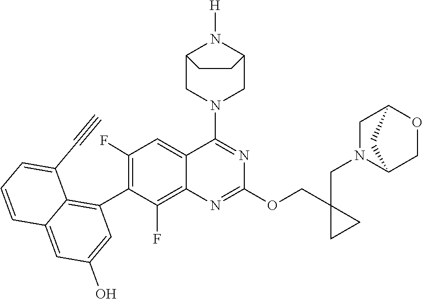 [SMILES: C#Cc1cccc2cc(O)cc(-c3c(F)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.337 min;

An atropisomer, which is afforded by chiral separation of the compound having the structure

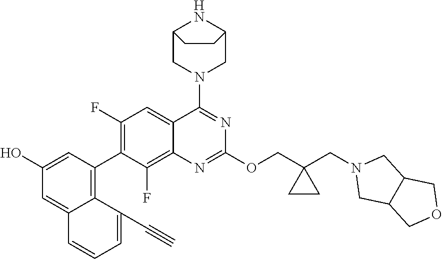 [SMILES: C#Cc1cccc2cc(O)cc(-c3c(F)cc4c(N5CC6CCC(C5)N6)nc(OCC5(CN6CC7COCC7C6)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.597 min;

An atropisomer, which is afforded by chiral separation of the compound having the structure

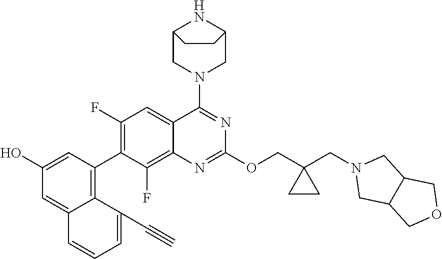 [SMILES: C#Cc1cccc2cc(O)cc(-c3c(F)cc4c(N5CC6CCC(C5)N6)nc(OCC5(CN6CC7COCC7C6)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.228 min;

An atropisomer, which is afforded by chiral separation of the compound having the structure

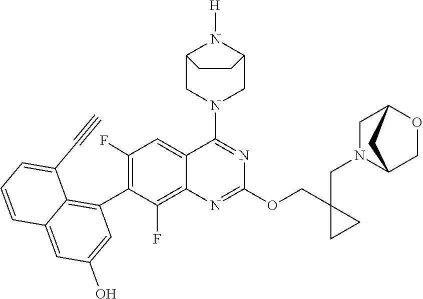 [SMILES: C#Cc1cccc2cc(O)cc(-c3c(F)cc4c(N5CC6CCC(C5)N6)nc(OCC5(CN6C[C@H]7C[C@@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 3.639 min;

An atropisomer, which is afforded by chiral separation of the compound having the structure

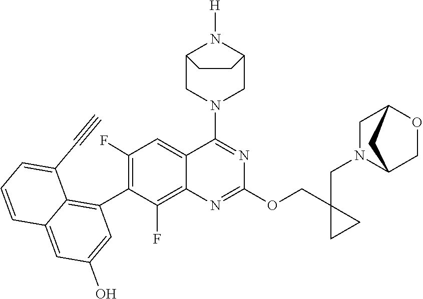 [SMILES: C#Cc1cccc2cc(O)cc(-c3c(F)cc4c(N5CC6CCC(C5)N6)nc(OCC5(CN6C[C@H]7C[C@@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 6.608 min;

An atropisomer, which is afforded by chiral separation of the compound having the structure

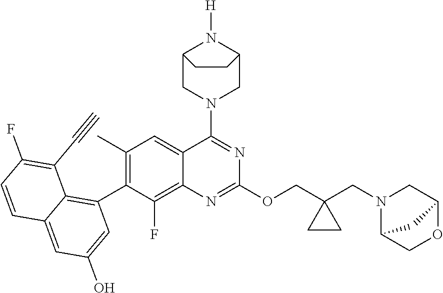 [SMILES: C#Cc1c(F)ccc2cc(O)cc(-c3c(C)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.217 min;

An atropisomer, which is afforded by chiral separation of the compound having the structure

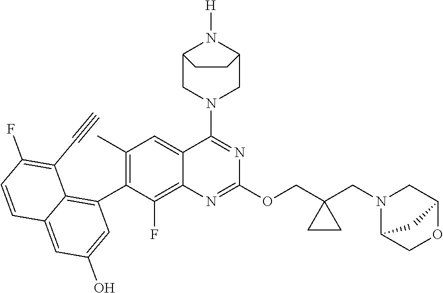 [SMILES: C#Cc1c(F)ccc2cc(O)cc(-c3c(C)cc4c(N5CC6CCC(C5)N6)nc(OCC5(CN6C[C@@H]7C[C@H]6CO7)CC5)nc4c3F)c12]

under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 5.886 min.

In another aspect, the present invention provides an intermediate selected from any one of the following formulas:

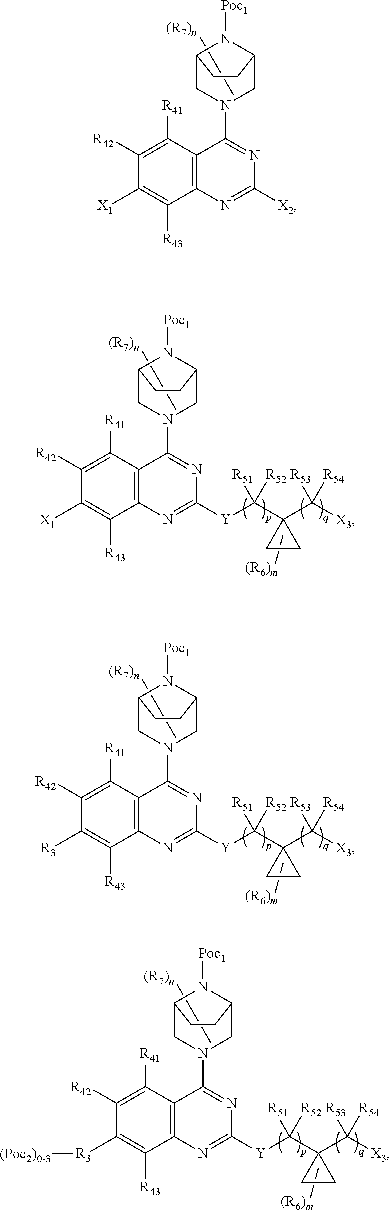

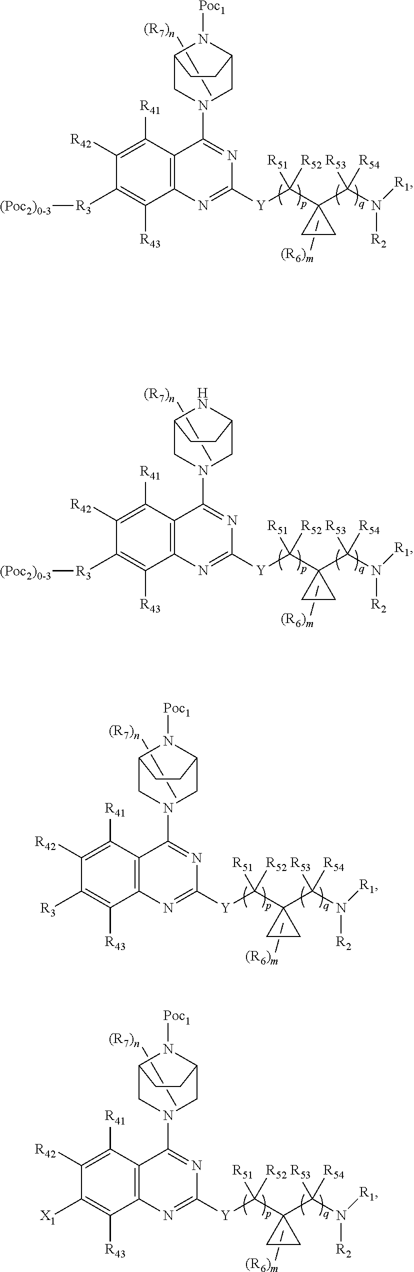

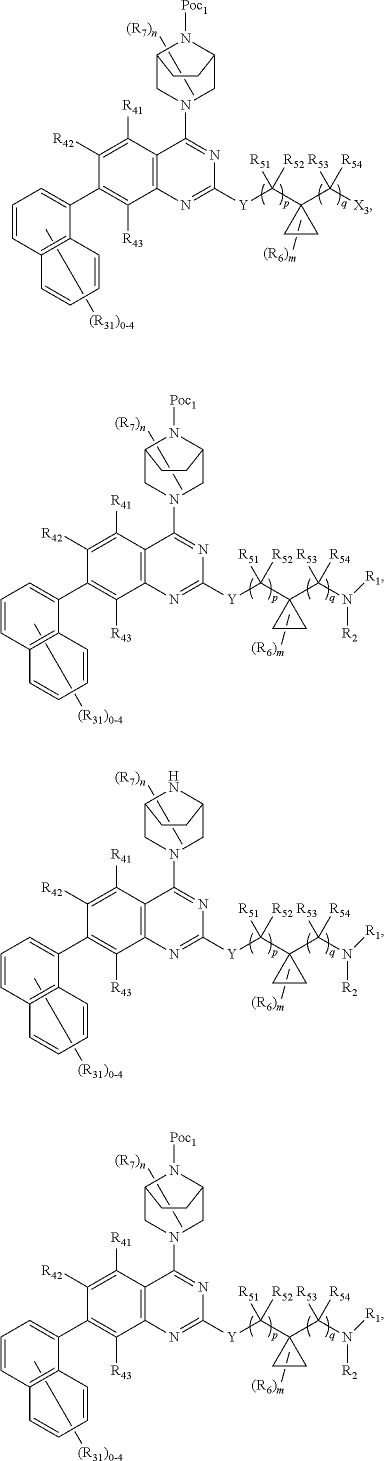

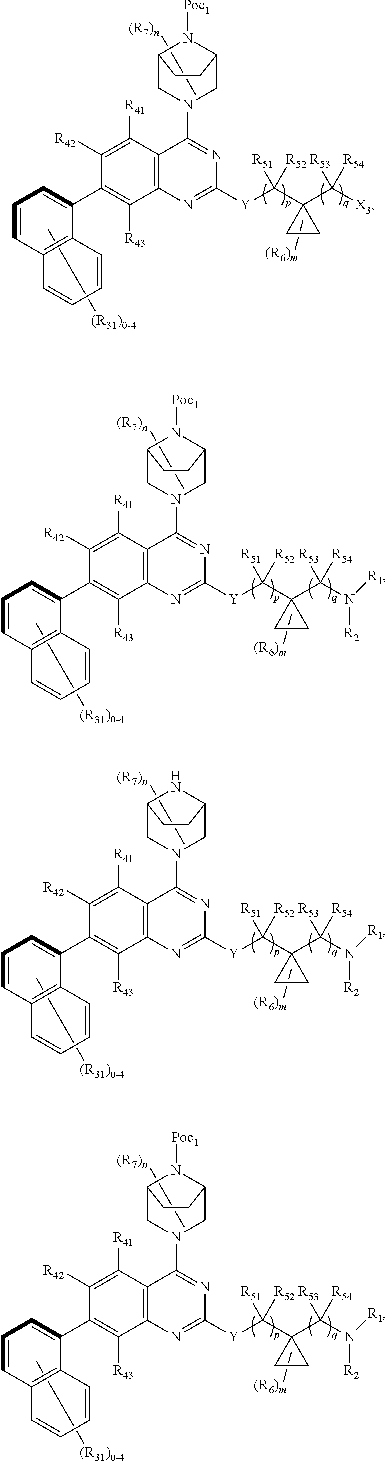

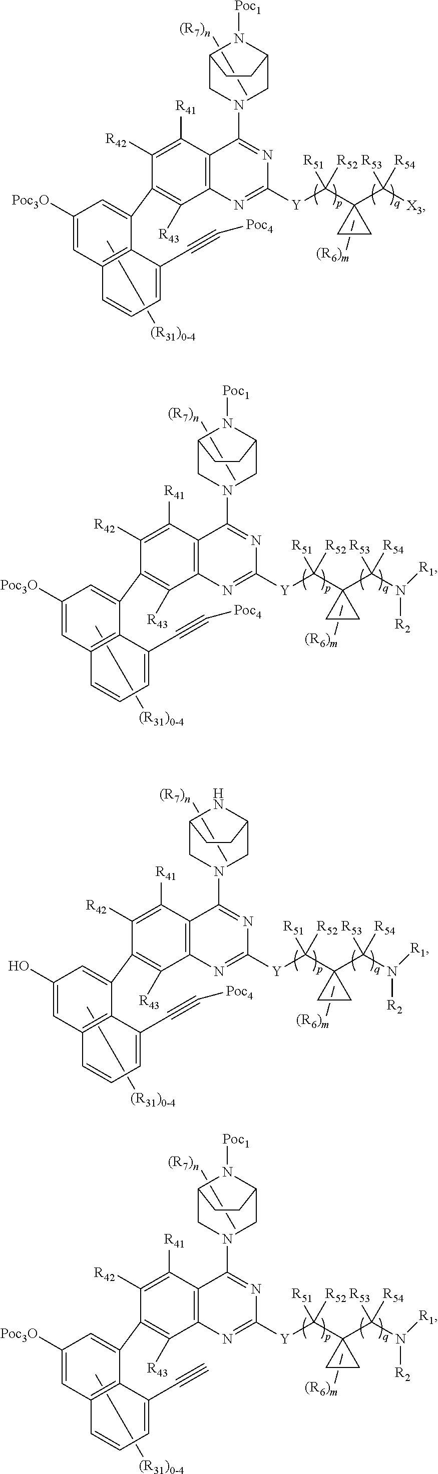

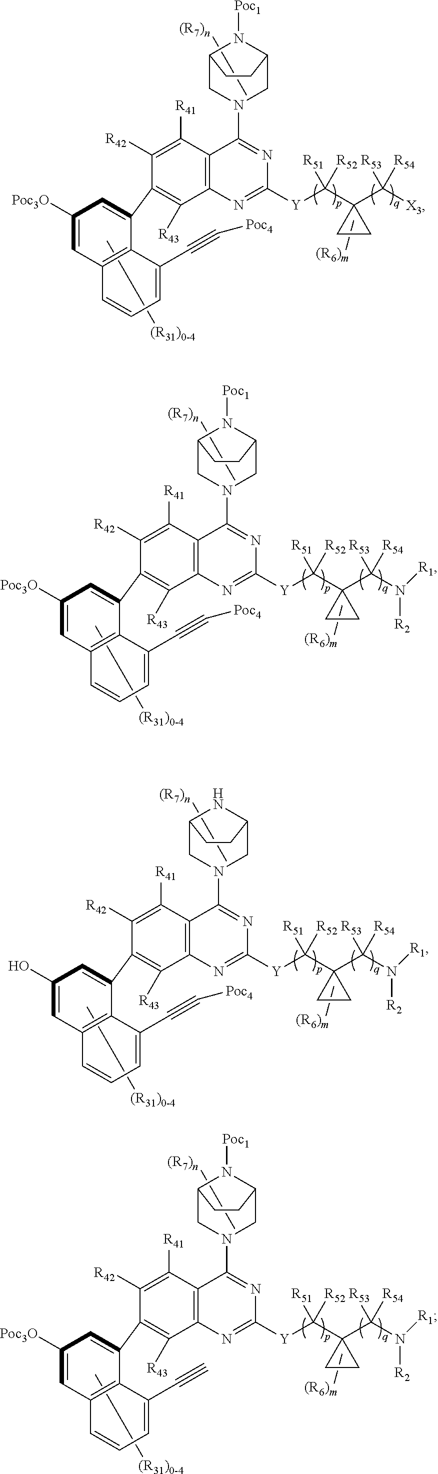

Wherein, the definition of R₁, R₂, R₃, R₃₁, R₄₁, R₄₂, R₄₃, R₅₁, R₅₂, R₅₃, R₅₄, R₆, R₇, Y, m, n, p or q in each of formulas is same as any one of [1] to [105];
X₁ in each of formulas is a leaving group or a group that can be converted to the leaving group, preferably, the leaving group is selected from halogen (such as —Cl, —Br or —I), —OS(═O)₂CH₃ or

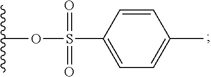 [SMILES: *OS(=O)(=O)c1ccc(C)cc1]

the group that can be converted to the leaving group is selected from —OH;
X₂ in each of formulas is a leaving group (such as —Cl, —Br or —I) or a group that can be converted to the leaving group, preferably, the leaving group is selected from halogen, —OS(═O)₂CH₃ or

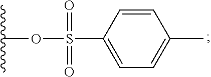 [SMILES: *OS(=O)(=O)c1ccc(C)cc1]

the group that can be converted to the leaving group is selected from —OH;
X₃ in each of formulas is a leaving group (such as —Cl, —Br or —I) or a group that can be converted to the leaving group, preferably, the leaving group is selected from halogen (such as —Cl, —Br or —I), —OS(═O)₂CH₃ or

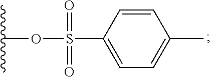 [SMILES: *OS(=O)(=O)c1ccc(C)cc1]

the group that can be converted to the leaving group is selected from —OH;
Poc₁ is the protecting group of the nitrogen atom, preferably, Poc₁ is t-Butyloxycarbonyl;
Poc₂ is the protecting group of R₃₁ substituted on the R₃;
Poc₃ is the protecting group of —OH, preferably, Poc₃ is methoxymethoxy;
Poc₄ is the protecting group of —C≡CH, preferably, Poc₄ is triisopropylsilyl;

Preferably, the intermediate is selected from:

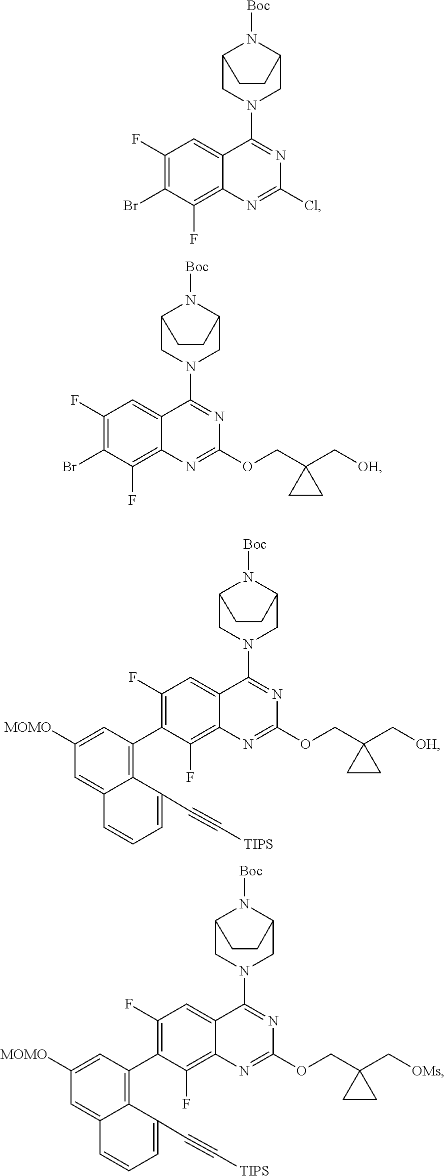

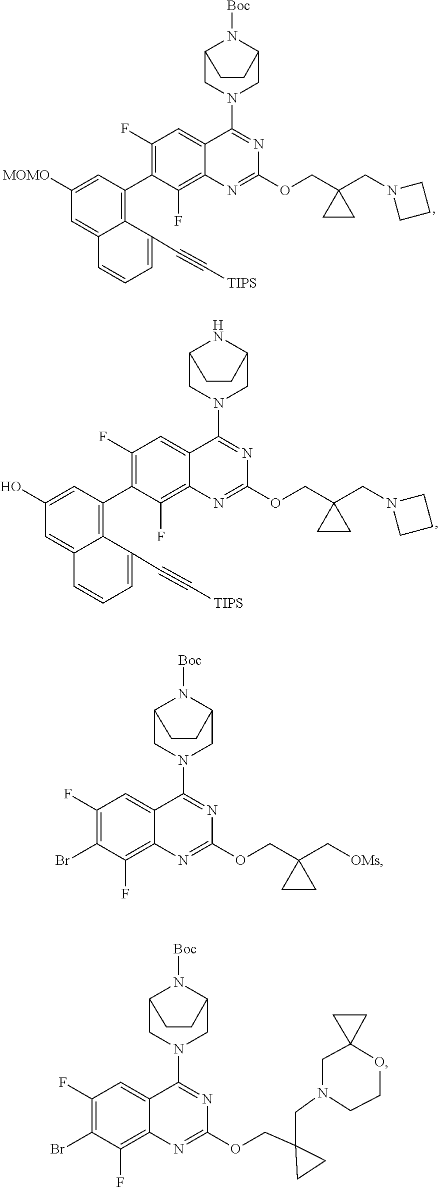

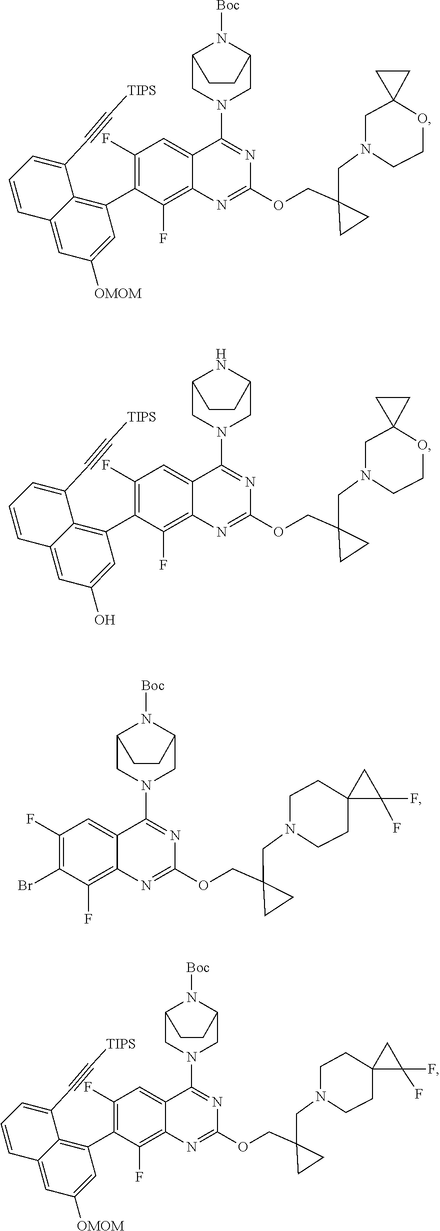

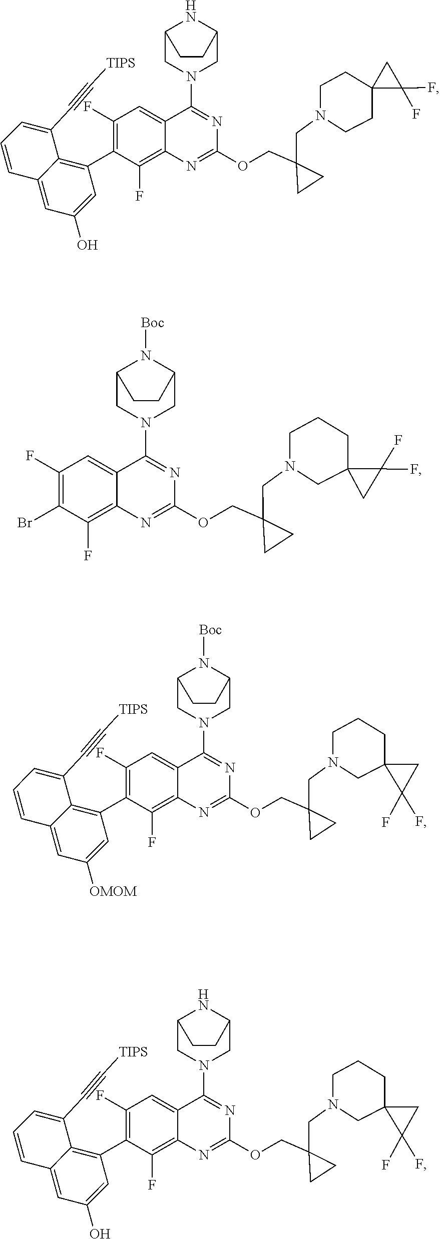

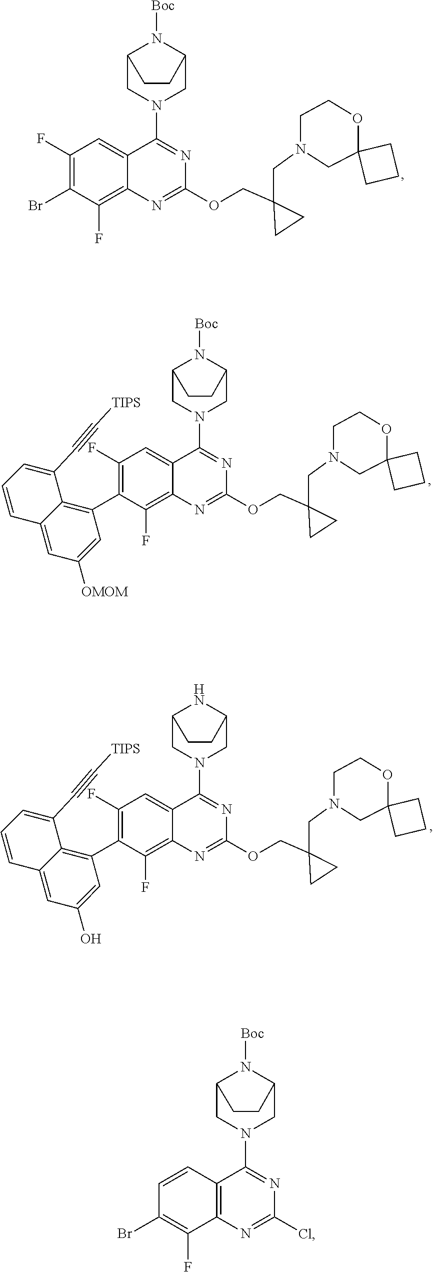

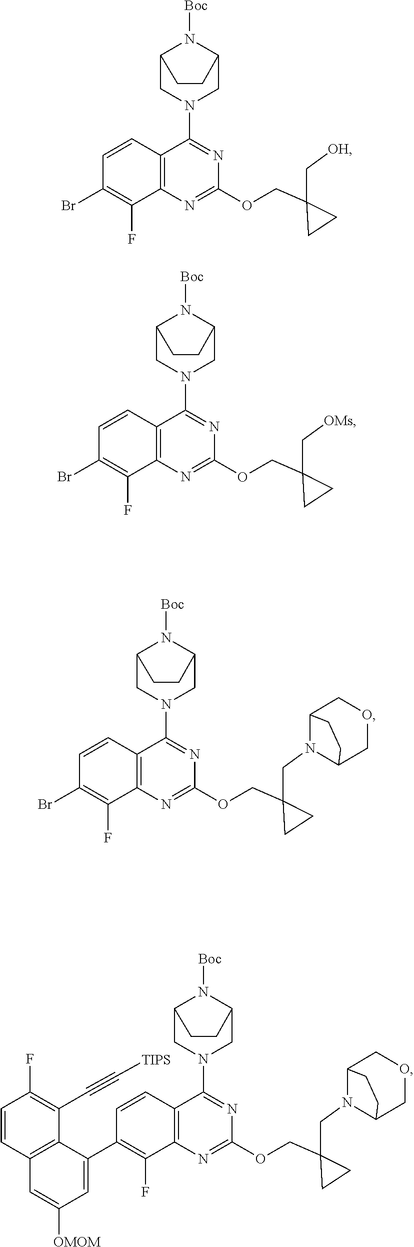

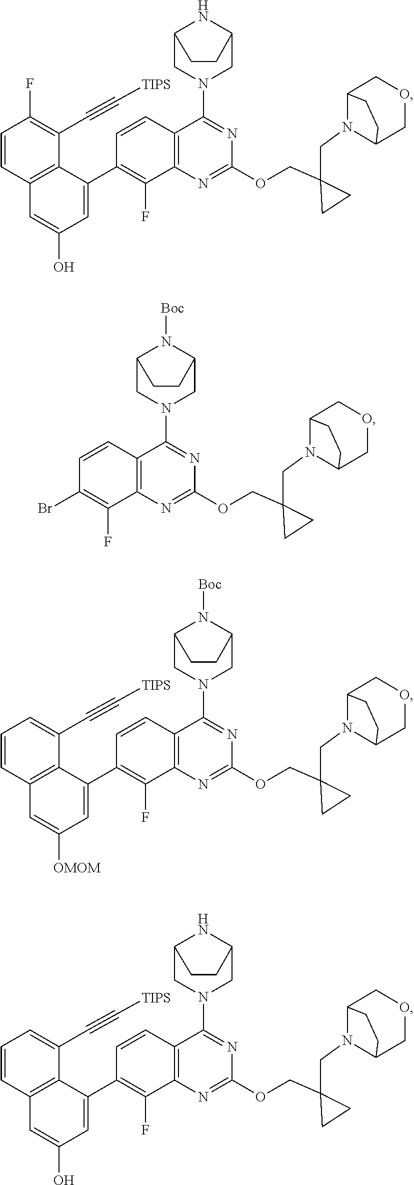

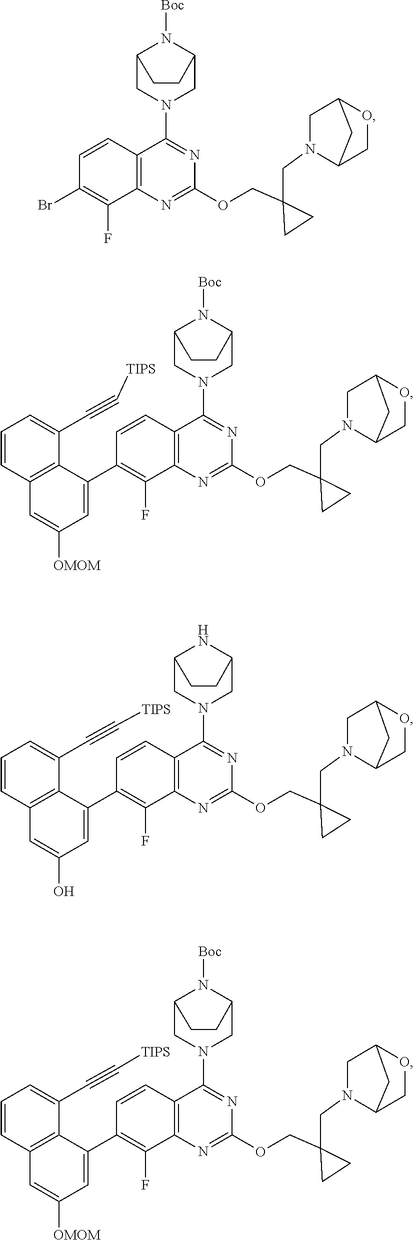

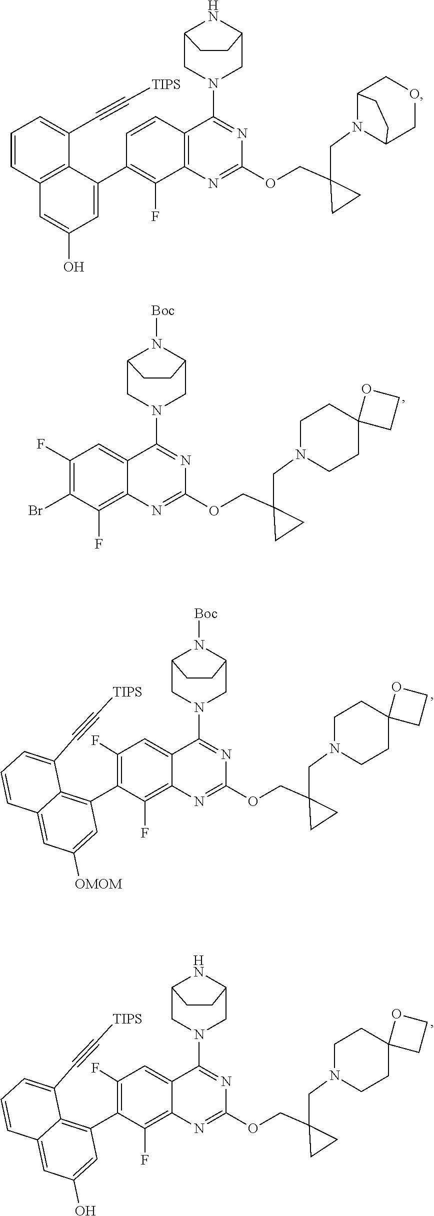

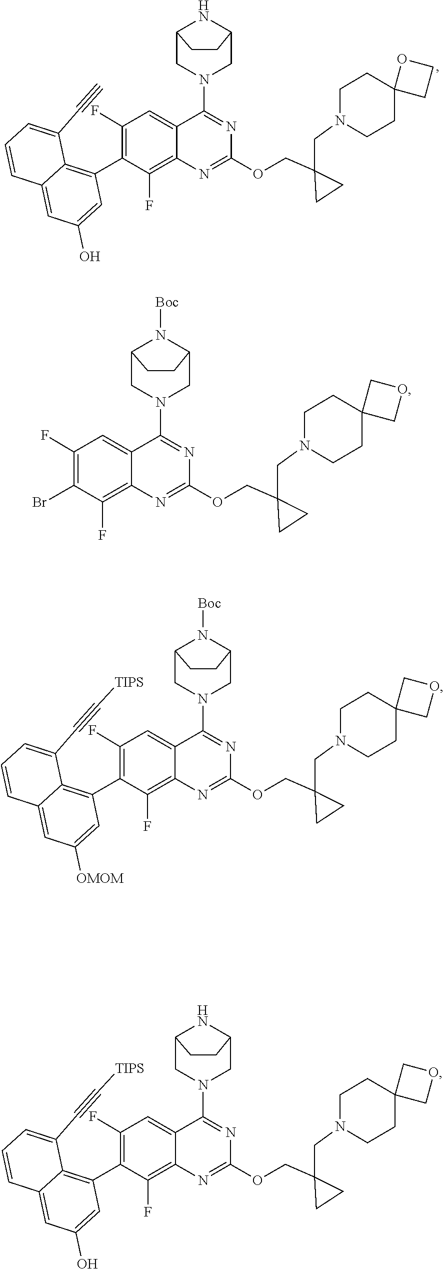

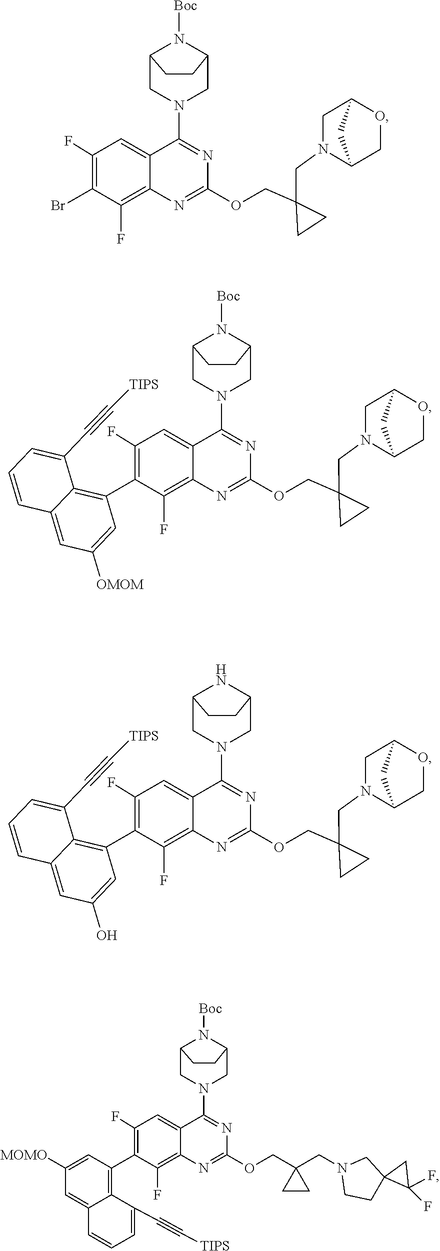

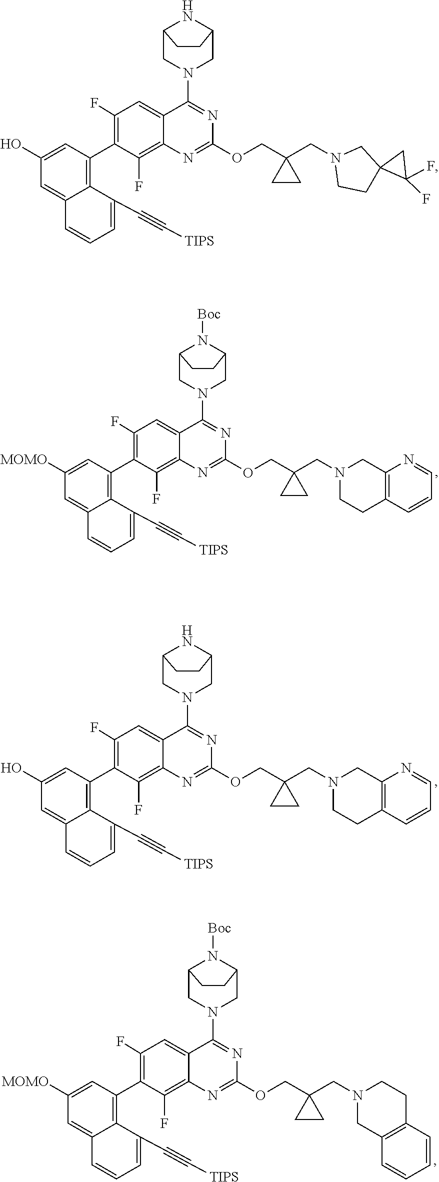

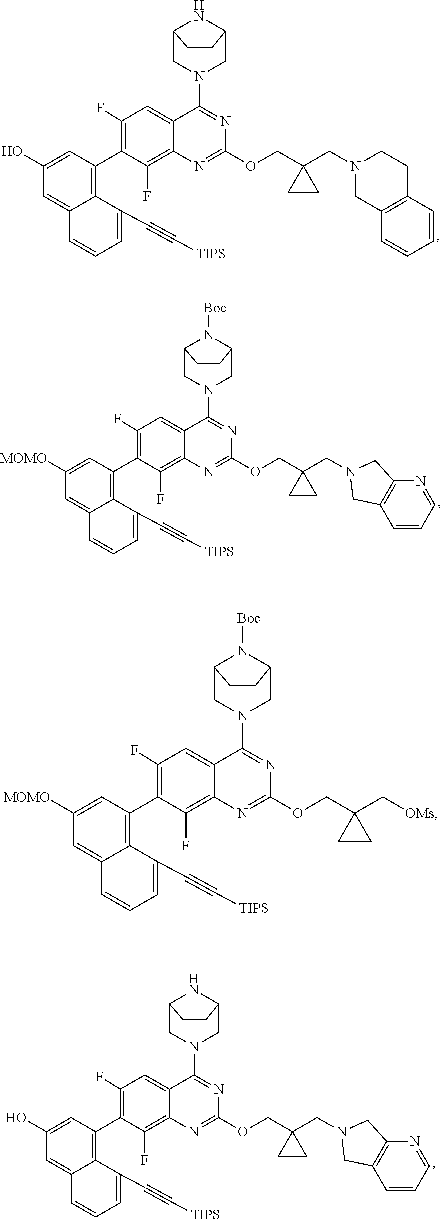

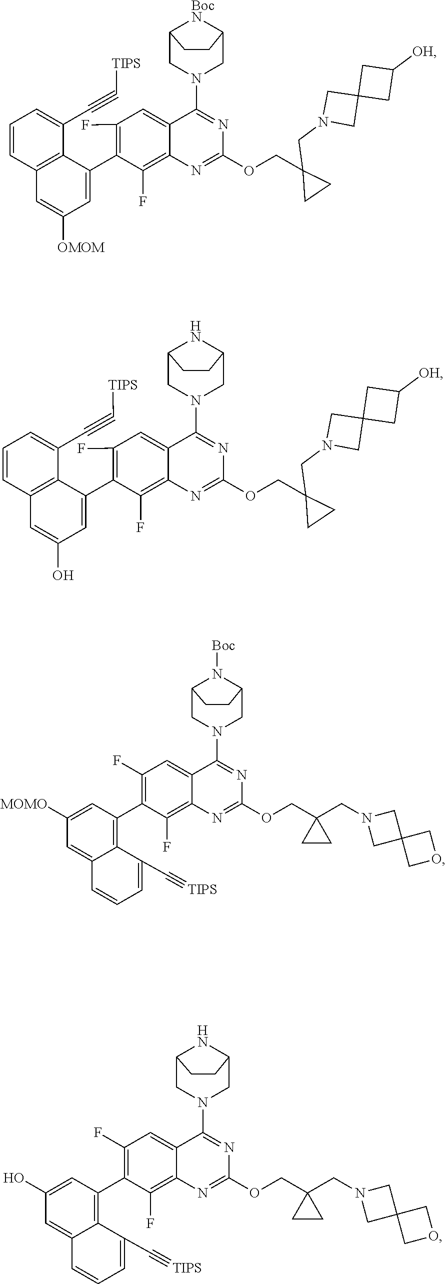

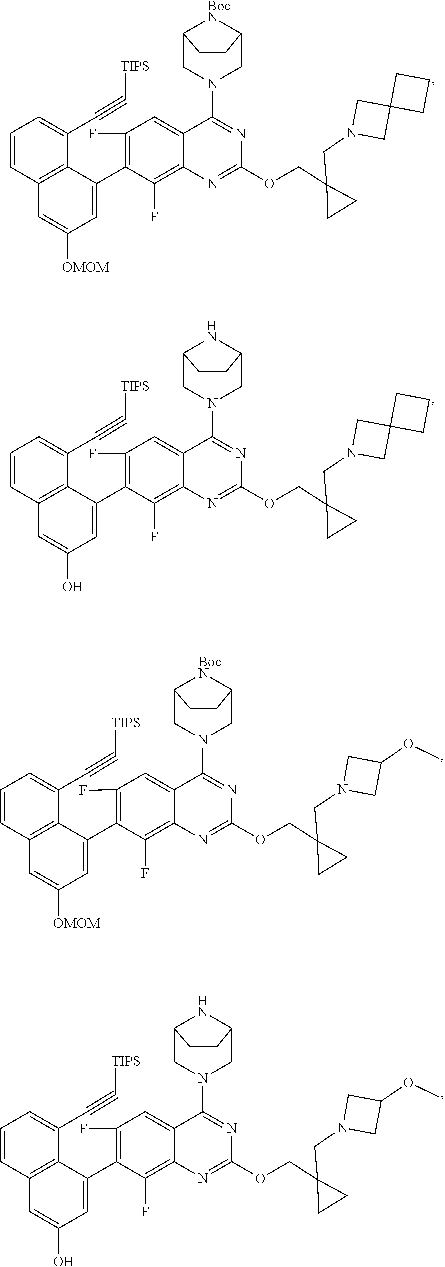

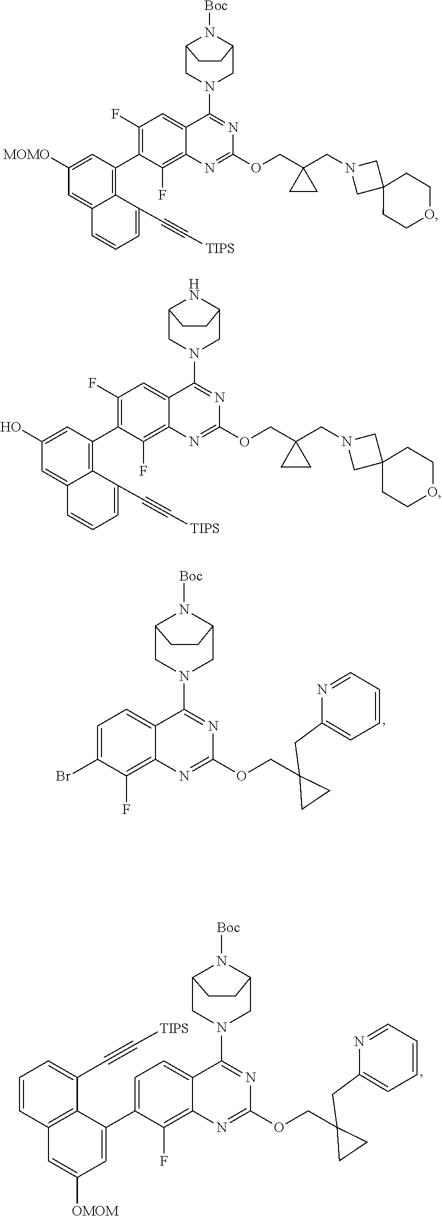

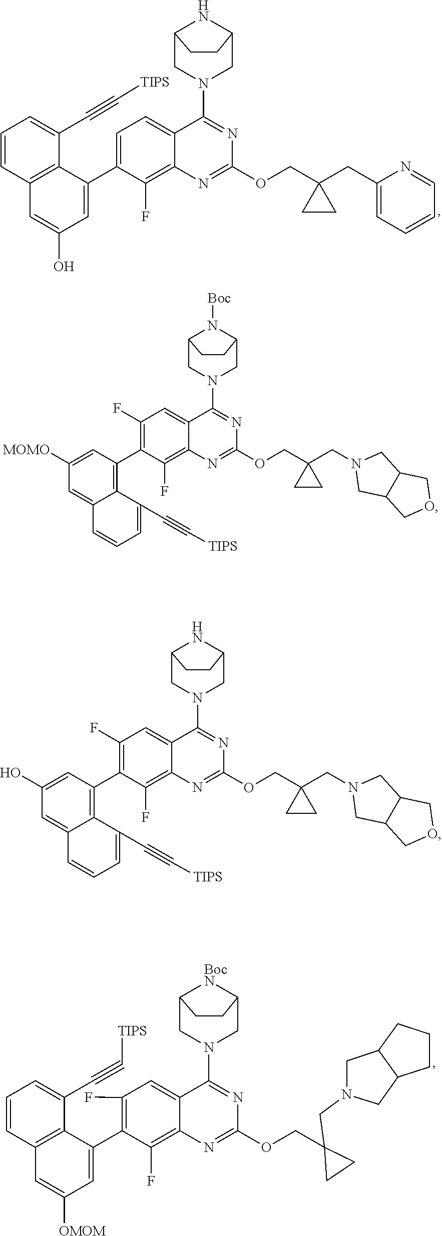

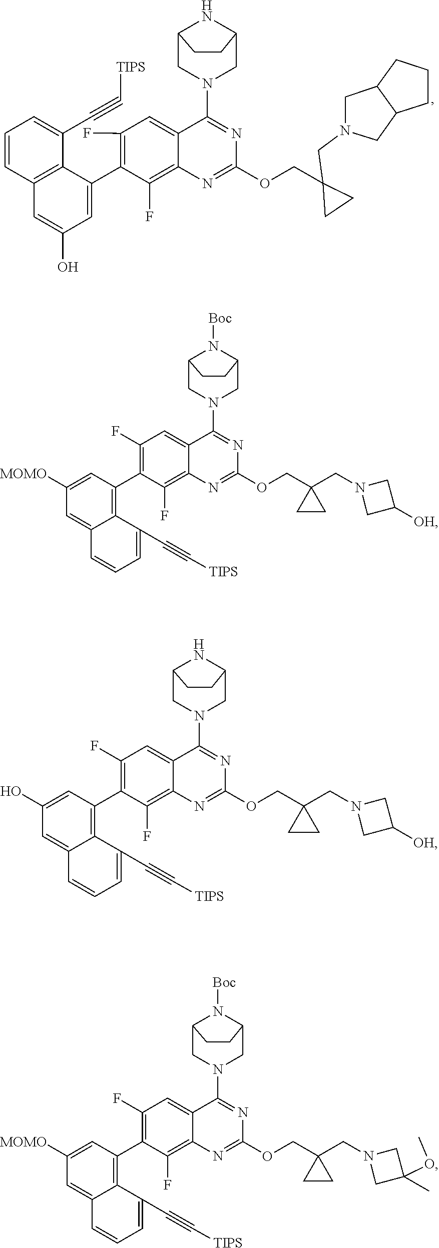

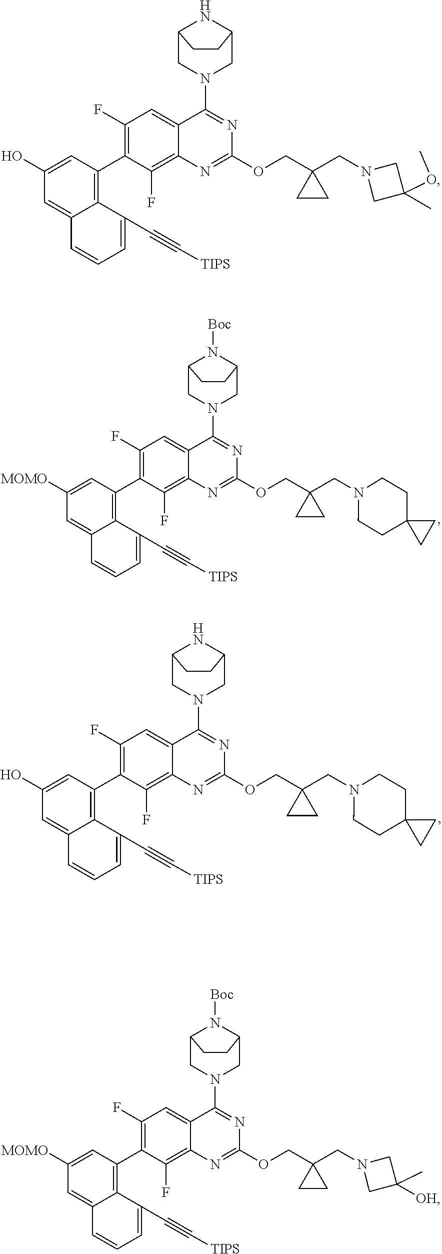

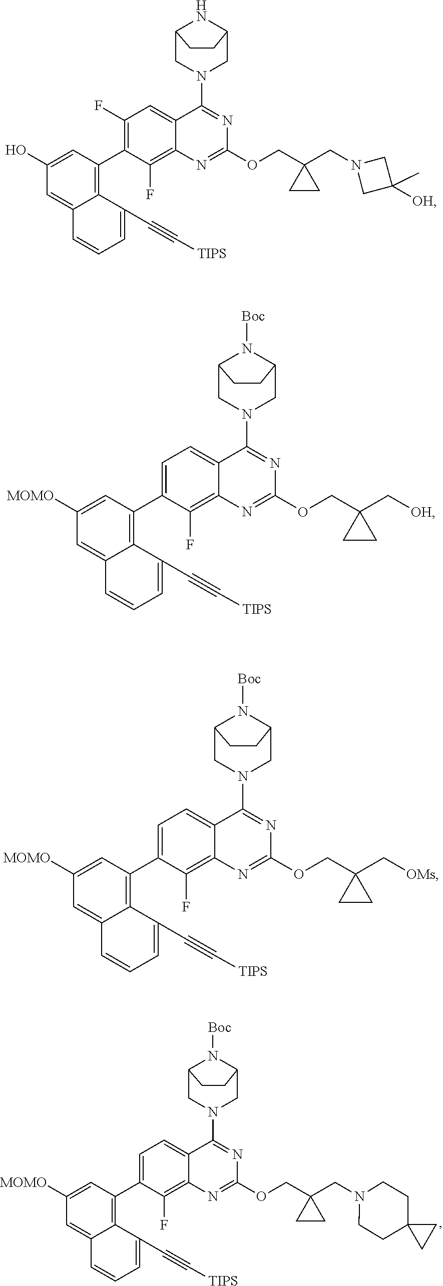

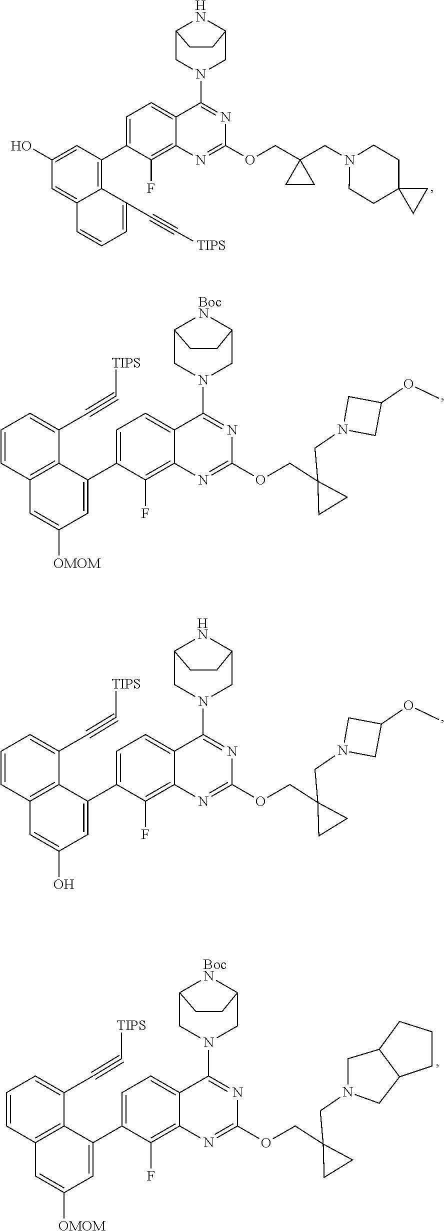

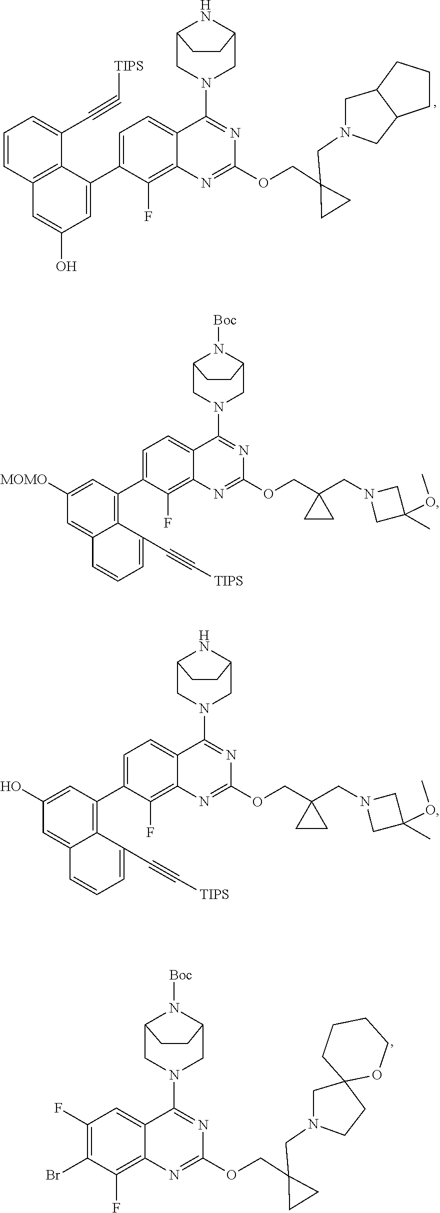

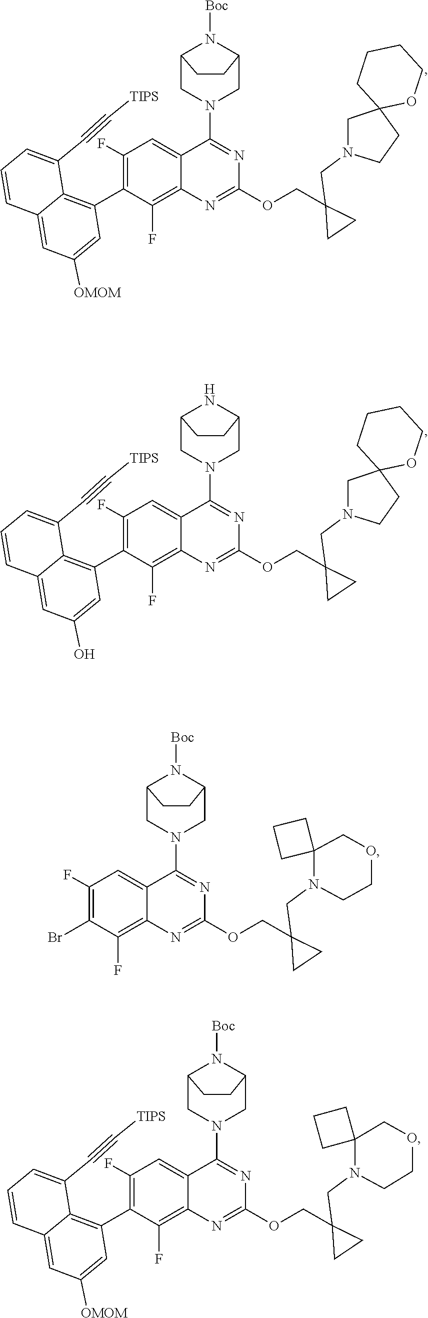

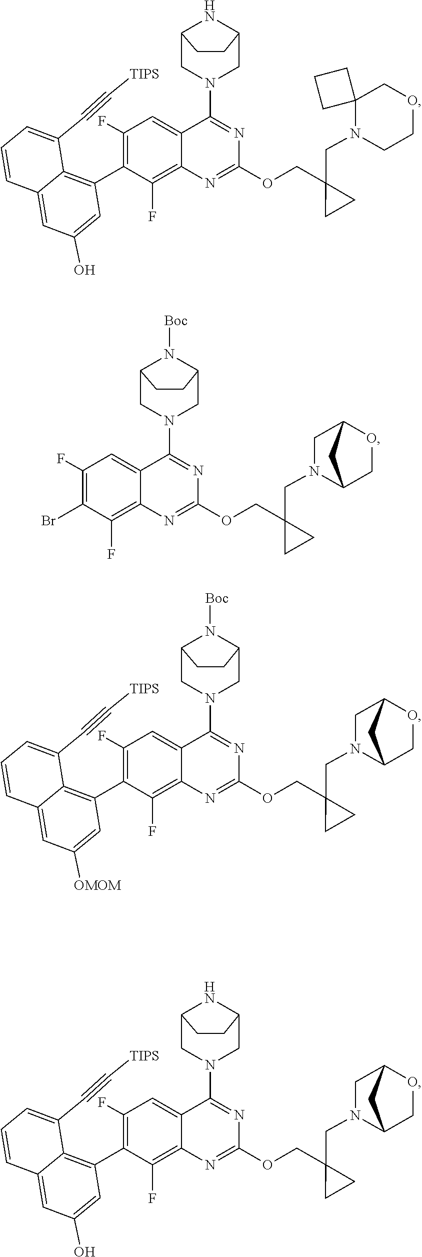

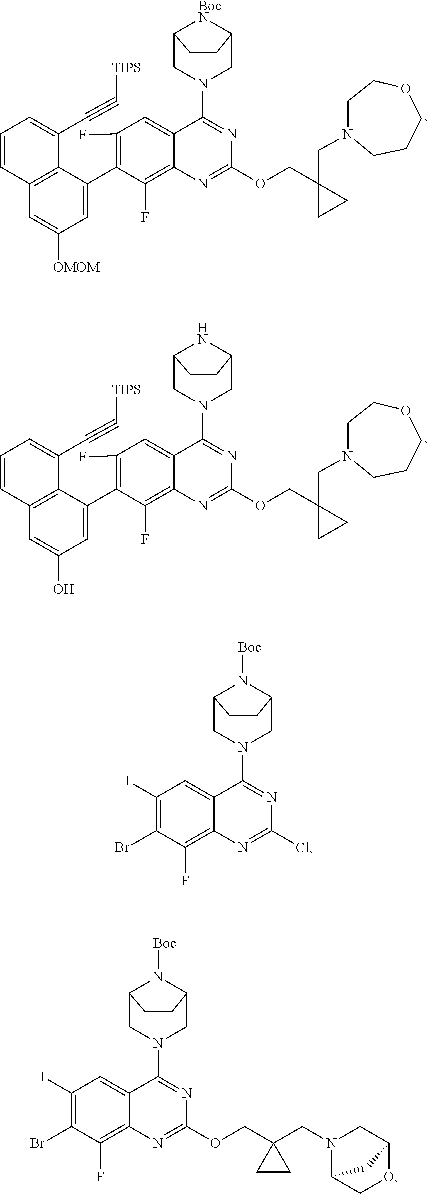

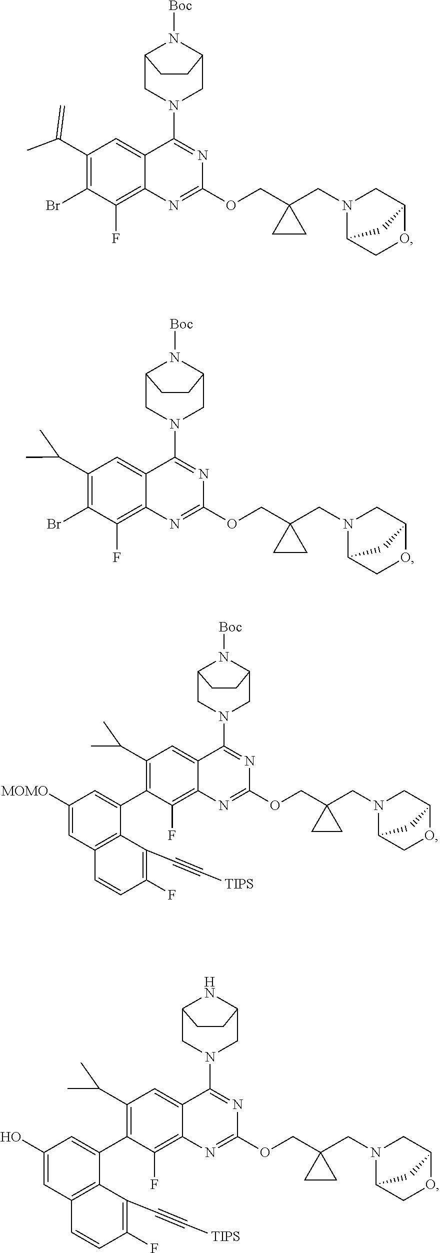

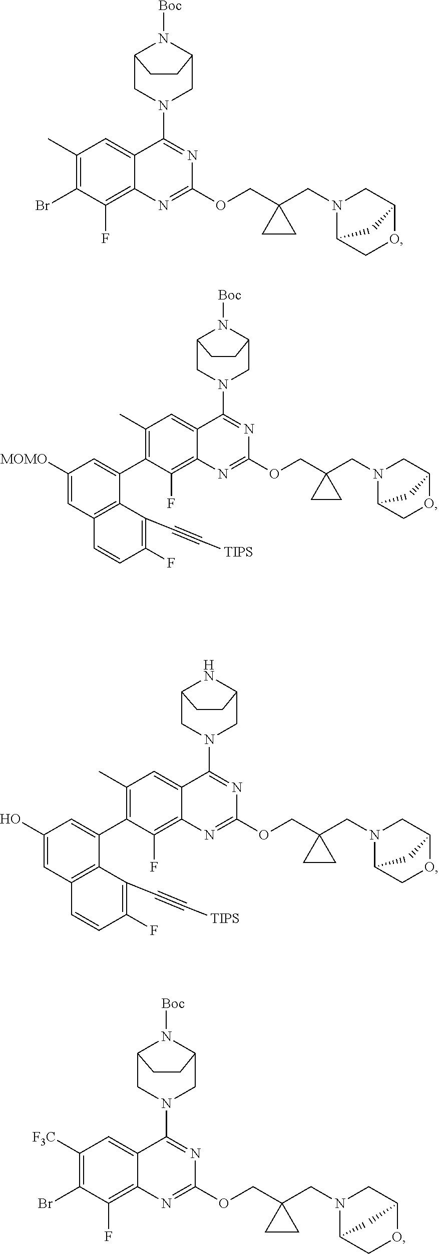

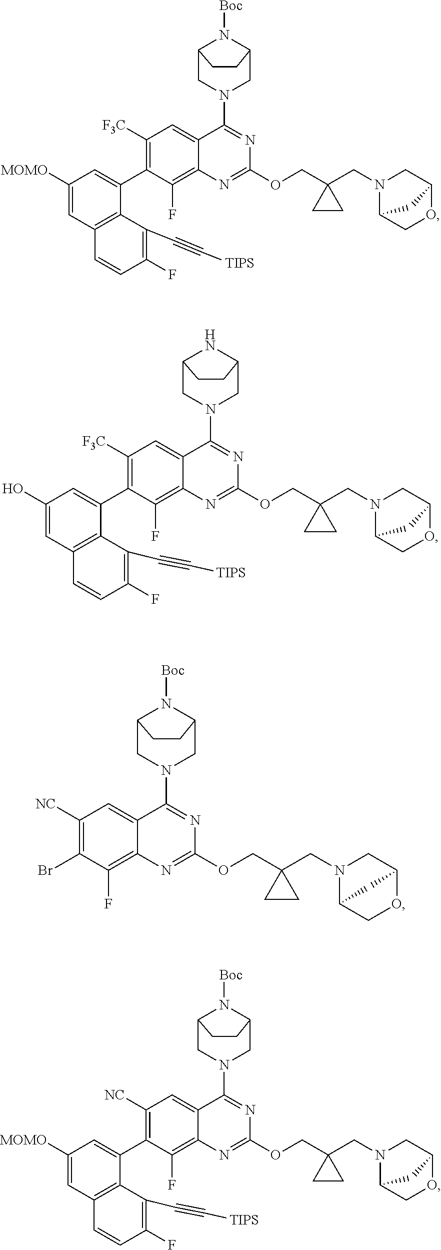

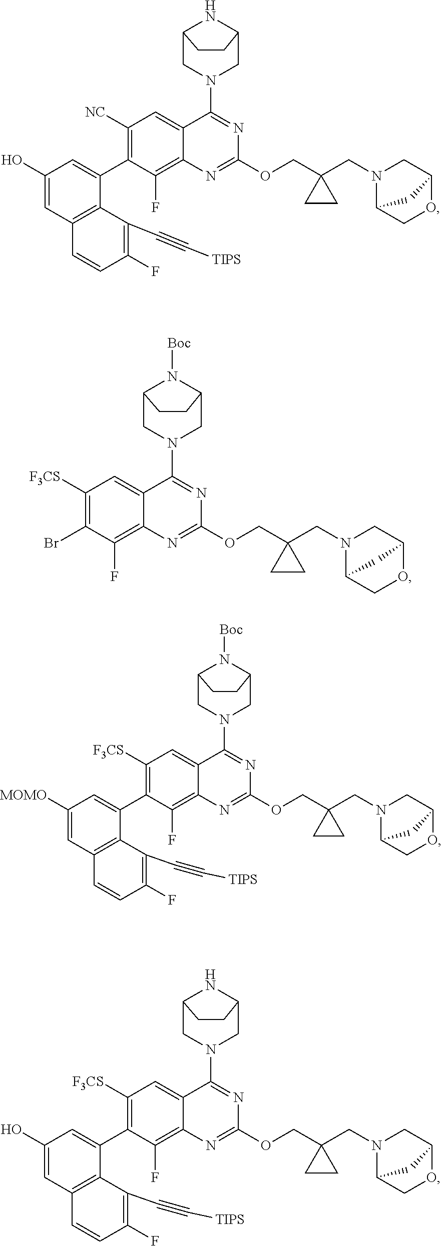

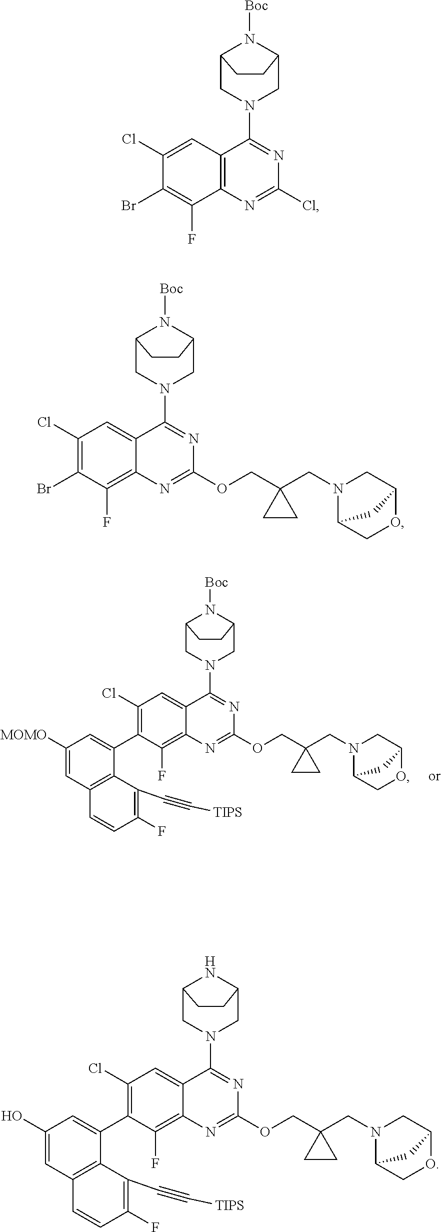

In another aspect, the present invention provides a process for preparing the compound of formula (I), a stereoisomer thereof, an atropisomer thereof, a deuterated derivative thereof, a tautomer thereof, a prodrug thereof, or a pharmaceutically acceptable salt thereof according to any one of [1] to [105], comprising the following scheme 1 or scheme 2:

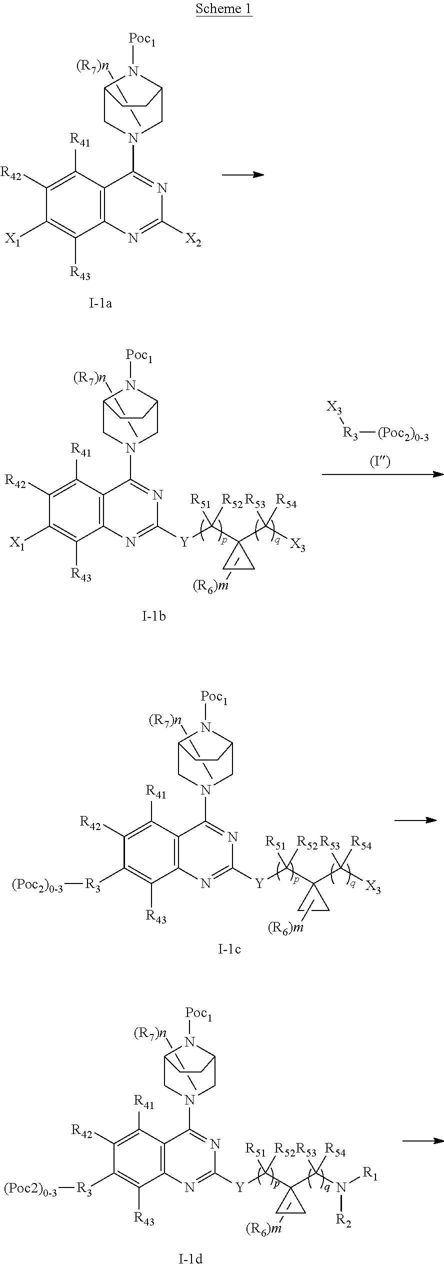

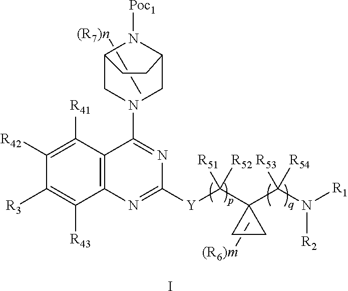

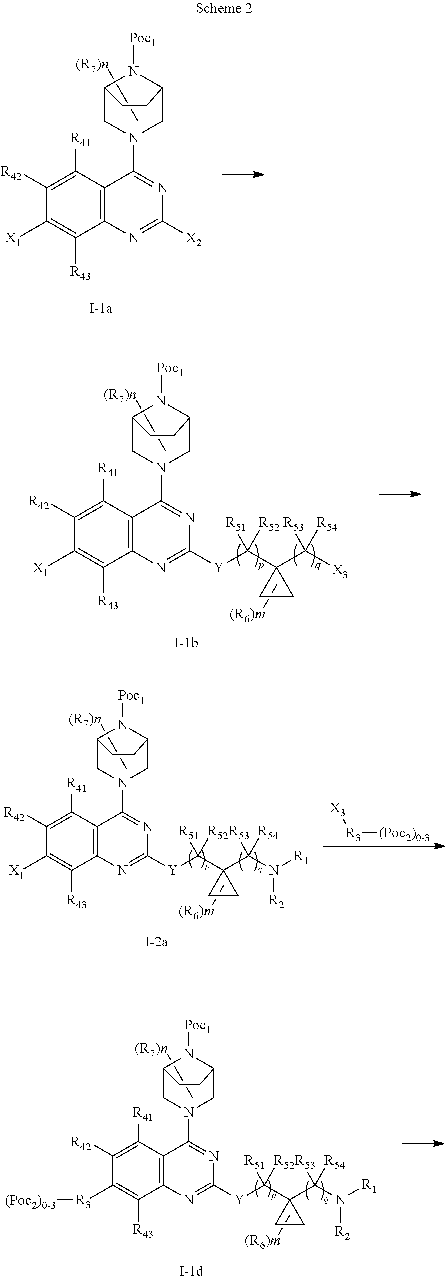

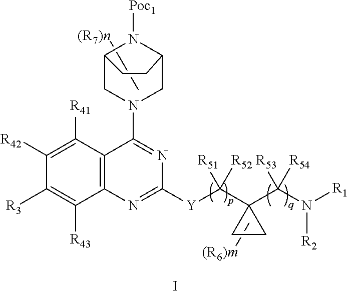

Wherein, the definition of R₁, R₂, R₃, R₃₁, R₄₁, R₄₂, R₄₃, R₅₁, R₅₂, R₅₃, R₅₄, R₆, R₇, Y, m, n, p or q in each of formulas is same as any one of [1] to [105];
X₁ in each of formulas is a leaving group or a group that can be converted to the leaving group, preferably, the leaving group is selected from halogen (such as —Cl, —Br or —I), —OS(═O)₂CH₃ or

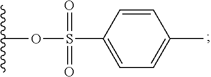 [SMILES: *OS(=O)(=O)c1ccc(C)cc1]

the group that can be converted to the leaving group is selected from —OH;
X₂ in each of formulas is a leaving group (such as —Cl, —Br or —I) or a group that can be converted to the leaving group, preferably, the leaving group is selected from halogen, —OS(═O)₂CH₃ or

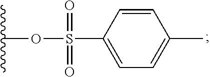 [SMILES: *OS(=O)(=O)c1ccc(C)cc1]

the group that can be converted to the leaving group is selected from —OH;
X₃ in each of formulas is a leaving group (such as —Cl, —Br or —I) or a group that can be converted to the leaving group, preferably, the leaving group is selected from halogen (such as —Cl, —Br or —I), —OS(═O)₂CH₃ or

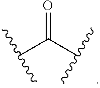 [SMILES: *C(*)=O]

the group that can be converted to the leaving group is selected from —OH;
Poc₁ is the protecting group of the nitrogen atom, preferably, Poc₁ is t-Butyloxycarbonyl;
Poc₂ is the protecting group of R₃₁ substituted on the R₃;
Poc₃ is the protecting group of —OH, preferably, Poc₃ is methoxymethoxy;
Poc₄ is the protecting group of —C≡CH, preferably, Poc₄ is triisopropylsilyl.

In another aspect, the present invention provides a proteolysis targeting chimeric (PROTAC) compound acting as a degradation modulator of KRAS G12D protein, wherein, said PROTAC compound is formed by joining the compound of formula (I), a stereoisomer thereof, an atropisomer thereof, a deuterated derivative thereof, a tautomer thereof, a prodrug thereof, or a pharmaceutically acceptable salt thereof according to any one of [1] to [105] with an E3 ubiquitin ligase ligand with or without a linker; preferably, with a linker.

In another aspect, the present invention provides a prodrug of the compound of formula (I), a stereoisomer thereof, an atropisomer thereof, a deuterated derivative thereof, a tautomer thereof, or a pharmaceutically acceptable salt thereof according to any one of [1] to [105].

In another aspect, the present invention provides a pharmaceutical composition comprising the compound of formula (I), a stereoisomer thereof, an atropisomer thereof, a deuterated derivative thereof, a tautomer thereof, a prodrug thereof, or a pharmaceutically acceptable salt thereof of the present invention; the proteolysis targeting chimeric (PROTAC) compound of the present invention; or the prodrug of the present invention; and at least one pharmaceutically acceptable excipient.

In another aspect, the present invention provides a use of the compound of formula (I), a stereoisomer thereof, an atropisomer thereof, a deuterated derivative thereof, a tautomer thereof, a prodrug thereof, or a pharmaceutically acceptable salt thereof of the present invention; the proteolysis targeting chimeric (PROTAC) compound of the present invention; the prodrug of the present invention; or the pharmaceutical composition of the present invention for the manufacture of a medicament for the treatment of diseases or conditions related to KRAS G12D protein; preferably, the diseases or conditions related to KRAS G12D protein is cancer related to KRAS G12D protein; more preferably, the cancer is selected from pancreatic cancer, colorectal cancer, endometrial cancer or lung cancer; further preferably, the lung cancer is selected from non-small cell lung cancer or small cell lung cancer.

In another aspect, the present invention provides a method of treating a subject having a diseases or conditions related to KRAS G12D protein, said method comprising administering to the subject a therapeutically effective amount of the compound of formula (I), a stereoisomer thereof, an atropisomer thereof, a deuterated derivative thereof, a tautomer thereof, a prodrug thereof, or a pharmaceutically acceptable salt thereof of the present invention; the proteolysis targeting chimeric (PROTAC) compound of the present invention; the prodrug of the present invention; or the pharmaceutical composition of the present invention; preferably, the diseases or conditions related to KRAS G12D protein is cancer related to KRAS G12D protein; more preferably, the cancer is selected from pancreatic cancer, colorectal cancer, endometrial cancer or lung cancer; further preferably, the lung cancer is selected from non-small cell lung cancer or small cell lung cancer.

In another aspect, the present invention provides a compound of formula (I), a stereoisomer thereof, an atropisomer thereof, a deuterated derivative thereof, a tautomer thereof, a prodrug thereof, or a pharmaceutically acceptable salt thereof of the present invention; or the proteolysis targeting chimeric (PROTAC) compound of the present invention; the prodrug of the present invention; or the pharmaceutical composition of the present invention for use in the treatment of diseases or conditions related to KRAS G12D protein. preferably, the diseases or conditions related to KRAS G12D protein is cancer related to KRAS G12D protein; more preferably, the cancer is selected from pancreatic cancer, colorectal cancer, endometrial cancer or lung cancer; further preferably, the lung cancer is selected from non-small cell lung cancer or small cell lung cancer.

In another aspect, the present invention provides a use of the compound of formula (I), a stereoisomer thereof, an atropisomer thereof, a deuterated derivative thereof, a tautomer thereof, a prodrug thereof, or a pharmaceutically acceptable salt thereof of the present invention as a targeting KRAS G12D protein ligand in a PROTAC compound acting as a degradation modulator of KRAS G12D protein.

### Definition

The term “halogen” or “halo”, as used interchangeably herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. C₁₋₆, in —C₁₋₆alkyl is defined to identify the group having 1, 2, 3, 4, 5 or 6 carbon atoms in a linear or branched arrangement.

The term “haloalkyl”, as used herein, unless otherwise indicated, means the above-mentioned alkyl substituted with one or more (for 1, 2, 3, 4, 5, or 6) halogen (—F, —Cl or —Br). In some embodiment, the haloalkyl is interchangeable —C₁₋₆haloalkyl or haloC₁₋₆alkyl, wherein, C₁₋₆ in the —C₁₋₆haloalkyl or haloC₁₋₆alkyl indicates that the total carbon atoms of the alkyl is 1 to 6. In some embodiments, the —C₁₋₆haloalkyl is the —C₁₋₃haloalkyl. In some embodiments, the —C₁₋₃haloalkyl is (methyl, ethyl, propyl or isopropyl) substituted with 1, 2, 3, 4, 5, or 6 —F; preferably, the —C₁₋₃haloalkyl is —CF₃.

The term “alkylene” means a difunctional group obtained by removal of an additional hydrogen atom from an alkyl group defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “—C₂₋₆alkenyl” contains from 2 to 6 carbon atoms. Alkenyl group include, but are not limited to, for example, ethenyl, propenyl, butenyl, 2-methyl-2-buten-1-yl, heptenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “haloalkoxy”, as used herein, unless otherwise indicated, means the above-mentioned alkoxy substituted with one or more (for 1, 2, 3, 4, 5, or 6) halogen (—F, —Cl or —Br). In some embodiment, the haloalkoxy is interchangeable —C₁₋₆haloalkoxy or haloC₁₋₆alkoxy, wherein, C₁₋₆ in the —C₁₋₆haloalkoxy or haloC₁₋₆alkoxy indicates that the total carbon atoms of the alkoxy is 1 to 6. In some embodiments, the —C₁₋₆haloalkoxy is the —C₁₋₃haloalkoxy. In some embodiments, the —C₁₋₃haloalkoxy is (methoxy, ethoxy, propoxy or isopropoxy) substituted with 1, 2, 3, 4, 5, or 6 —F; preferably, the —C₁₋₃haloalkoxy is —OCF₃.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The interchangeable term “heterocyclyl” or “heterocyclic”, as used herein, unless otherwise indicated, refers to unsubstituted and substituted mono or polycyclic non-aromatic ring system containing one or more heteroatoms, which comprising monocyclic heterocyclyl ring, bicyclic heterocyclyl ring, bridged heterocyclyl ring, fused heterocyclyl ring or spiro heterocyclyl ring. Preferred heteroatoms include N, O, and S, including N-oxides, sulfur oxides, and dioxides. Preferably, the ring is three to ten membered and is either fully saturated or has one or more degrees of unsaturation. Multiple degrees of substitution, preferably one, two or three, are included within the present definition. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinylsulfoxide, thiamorpholinylsulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclicheteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “carbocyclic” refers to a substituted or unsubstituted monocyclic ring, bicyclic ring, bridged ring, fused ring, spiro ring non-aromatic ring system only containing carbon atoms. Preferably, the ring is three to ten membered and is either fully saturated or has one or more degrees of unsaturation. Multiple degrees of substitution, preferably one, two or three, are included within the present definition. The carbocyclic includes but not be limited cycloalkyl, cycloalkenyl and cycloalkynyl. Exemplary “cycloalkyl” groups includes but not limited to, cyclopropyl, cyclobutyl, cyclopentyl, cyclohexyl and the like.

The term “one or more” refers to one or more than one. In some embodiments, “one or more” refers to 1, 2, 3, 4, 5 or 6. In some embodiments, “one or more” refers to 1, 2, 3 or 4. In some embodiments, “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

When one or more substituents are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a spiro ring, each of substituents may be respectively independently substituted on every ring atom of the polycyclic ring.

The term “oxo” refers to oxygen atom together with the attached carbon atom forms the group

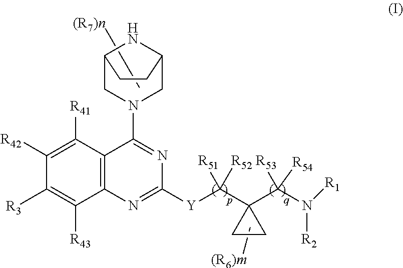

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The invention includes all possible stereoisomers of the compound.

Certain of the compounds provided herein may exist as atropisomers, which are conformational stereoisomers that occur when rotation about a single bond in the molecule is prevented, or greatly slowed, as a result of steric interactions with other parts of the molecule. The compounds provided herein include all atropisomers, both as pure individual atropisomer preparations, enriched preparations of each, or a non-specific mixture of each. Where the rotational barrier about the single bond is high enough, and interconversion between conformations is slow enough, separation and isolation of the isomeric species may be permitted.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

The term “deuterated derivative”, used herein, unless otherwise indicated, refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivative described herein. Thus, unless otherwise stated, when a reference is made to a “deuterated derivative” of a compound of the disclosure, at least one hydrogen is replaced with deuterium at well above its natural isotopic abundance (which is typically about 0.015%) In some embodiments, the deuterated derivative of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

When a tautomer of the compound in the present invention exists, the present invention includes any possible tautomers and pharmaceutically acceptable salts thereof, and mixtures thereof, except where specifically stated otherwise.

The compounds described herein can also inhibit KRAS G12D protein function through incorporation into agents that catalyze the destruction of KRAS G12D protein. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. Preferably, the portion of the PROTAC that engages the E3 ligase is connected to the portion of the PROTAC that engages the target protein via a linker which consists of a variable chain of atoms. Recruitment of KRAS G12D protein to the E3 ligase will thus result in the destruction of the KRAS G12D protein. The variable chain of atoms can include, for example, rings, heteroatoms, and/or repeating polymeric units. It can be rigid or flexible. It can be attached to the two portions described above using standard techniques in the art of organic synthesis.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds in present invention or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of Formula I or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg, 1000 mg, 1500 mg or 2000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Unless otherwise apparent from the context, when a value is expressed as “about” X or “approximately” X, the stated value of X will be understood to be accurate to ±10%, preferably, ±5%, ±2%.

These and other aspects will become apparent from the following written description of the invention.

### Methods of Preparation

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following Examples are provided to better illustrate the present invention. All parts and percentages are by weight and all temperatures are degrees Celsius, unless explicitly stated otherwise. The following abbreviations have been used in the examples:

| | |
|---|---|
| DMF | N,N-Dimethylformamide |
| EA, EtOAc | Ethyl acetate |
| Hex | n-Hexane |
| MeOH | Methanol |
| DCM | Dichloromethane |
| DCE | 1,2-Dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene]di- |
| | chloropalladium(II) |
| TFA | 2,2,2-Trifluoroacetic acid |
| RT/R.T | Room temperature |
| min(s) | minute(s) |
| h/hr(s) | hour(s) |
| aq | aqueous |
| Sat. | saturated |
| TLC | Thin layer chromatography |
| Prep - TLC | Preparative thin layer chromatography |
| MOMO | Methoxymethoxy |
| TIPS | Triisopropylsilyl |
| IPA.M, MIPA, or | Isopropylamine |
| IPAMine | |
| MsCl | Methanesulfonyl chloride |
| TEA, Et₃N | Triethanolamine |
| NIS | N-Iodosuccinimide |
| DMSO | Dimethyl sulfoxide |
| NCS | N-chlorosuccinimide |
| TBSCl | Tert-butyldimethylsilyl chloride\| |
| TMSCl | Trimethylsilyl chloride |
| MOMCl | Methoxymethyl chloride |
| LAH | Lithium aluminum hydride |
| LDA | Lithium diisopropylamide |
| LiHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | N,N-dimethylethanolamine |
| M-CPBA | 3-Chloroperbenzoic acid |
| NMP | N-methylpyrrolidone |
| AcOK | Potassium acetate |
| Tf₂O | Trifluoromethanesulfonic anhydride |

### Intermediate A1 (INT A1)

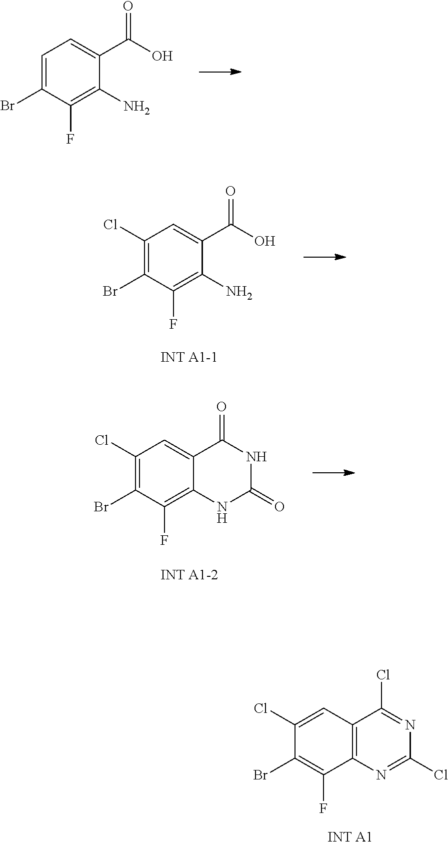

Following the procedure of WO2016164675, INT A1 was synthesized with 2-amino-4-bromo-3-fluorobenzoic acid as starting material.

### Intermediate A2 (INT A2)

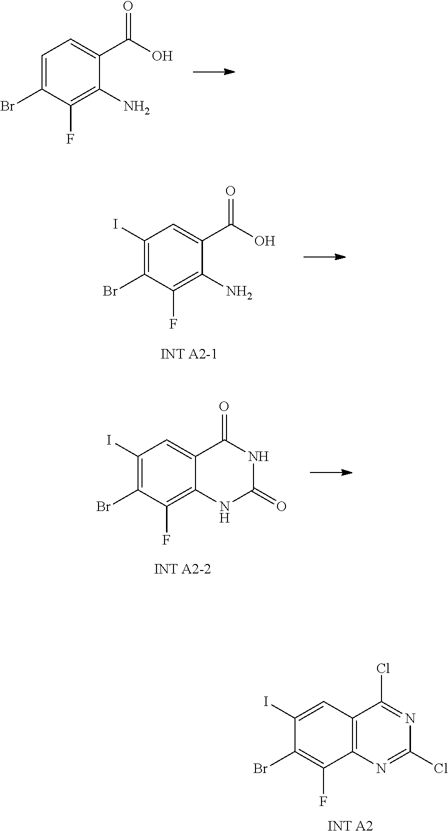

Following the procedure of WO2018143315, INT A2 was synthesized with 2-amino-4-bromo-3-fluorobenzoic acid as starting material.

### Intermediate A3 (INT A3)

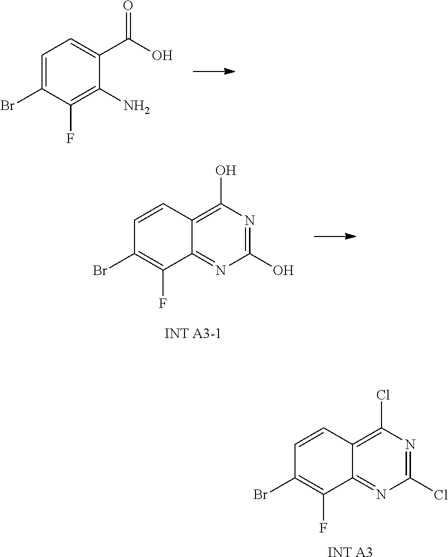

To a solution of 2-amino-4-bromo-3-fluorobenzoic acid (40.40 g, 172.63 mmol) in DCM (650 mL) was added dropwise sulfurisocyanatidic chloride (57.46 g, 405.98 mmol) at 0° C. The reaction mixture was stirred at RT for 5 hrs under an atmosphere of nitrogen, and then concentrated under reduced pressure to obtain a residue. A mixture of the residue and hydrochloric acid (6 N, 600 mL) was stirred overnight at 45° C. under atmosphere of nitrogen, and then filtered. The filter cake was washed with water (200 mL) and Hex (200 mL) successively, and then dried overnight at 45° C. under vacuum to afford INT A3-1 (37.63 g, 145.27 mmol). MS m/z: 257/259 [M−1]⁻.

To a solution of INT A3-1 (37.63 g, 145.27 mmol) in phosphorus oxychloride (115 mL) was added DIEA (46.24 g, 357.78 mmol) dropwise at 0° C. The reaction mixture was stirred at 110° C. for 2 hrs under an atmosphere of nitrogen, and then concentrated under vacuum to obtain a residue. The residue was dissolved in DCM (600 mL) and washed with water (400 mL). The organic layer was concentrated under vacuum to obtain a crude product which was dispersed in Hex:EA (400 mL, 20:1, v/v), and the resulting mixture was filtrated. The filter cake was washed with Hex (100 mL) and dried overnight at 40° C. under vacuum to afford INT A3 (31.29 g, 105.74 mmol) as a yellow solid.

### Intermediate A4 (INT A4)

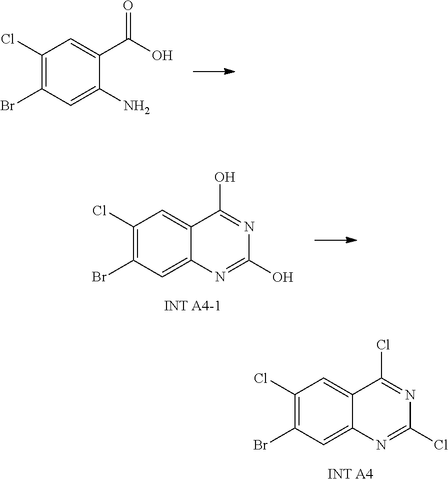

Sulfurisocyanatidic chloride (6.55 g, 46.28 mmol) was added dropwise to a solution of 2-amino-4-bromo-5-chlorobenzoic acid (5.12 g, 20.44 mmol) in DCM (30 mL) at 0° C. The reaction mixture was stirred for 3 hrs at R.T under an atmosphere of nitrogen, and then concentrated under reduced pressure to obtain a residue. A mixture of the residue and aqueous hydrochloric acid solution (1N, 40 mL) was stirred overnight under an atmosphere of nitrogen at 100° C., cooled to room temperature, and then filtered. The filter cake was washed successively with water (30 mL) and Hex (300 mL), dried overnight under vacuum at 45° C. to afford INT A4-1 (5.03 g, 18.26 mmol). MS m/z: 273 [M−H]⁻.

DIPEA (12.26 g, 94.86 mmol) was added dropwise to a mixture of INT A4-1 (5 g, 18.15 mmol) and phosphorus oxychloride (36 mL) at 0° C. The reaction mixture was stirred for 2.5 hrs at 100° C. under an atmosphere of nitrogen, and then concentrated under reduced pressure to obtain a residue. The residue was dissolved with DCM (600 mL) and washed with ice/water (400 mL), and the organic layer was concentrated under reduced pressure to obtain a crude product which was purified with silica gel chromatography to afford the INT A4 (1.6 g, 5.12 mmol).

### Intermediate A5 (INT A5)

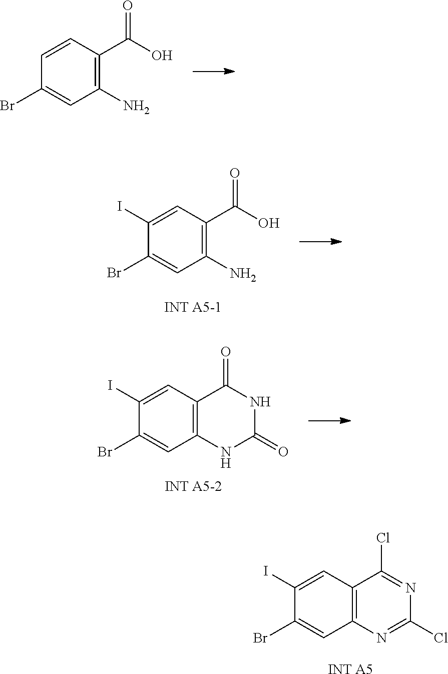

A solution of KI in water (3.93 g, 23.67 mmol) was added dropwise to a mixture of 2-amino-4-bromobenzoic acid (5.06 g, 23.42 mmol), NaCl (2.80 g, 47.91 mmol), NaIO₄ (5.00 g, 23.38 mmol) and AcOH (110 mL). The reaction mixture was stirred for 22 hrs at R.T, then poured into ice/water, diluted with saturated aq. Na₂S₂O₃, and extracted with DCM (3×100 mL). The organic layers were combined, washed with saturated aq. NaHCO₃, dried over Na₂SO₄ and concentrated under reduced pressure to obtain a residue which was purified with silica gel chromatography (eluting with EA) to afford INT A5-1 (6.87 g, yield 85.78%) as a brown solid. MS m/z: 340 [M−H]⁻.

Chlorosulfonyl isocyanate (6.92 g, 48.89 mmol) was added dropwise to a solution of INT A5-1 (6.87 g, 20.09 mmol) in DCM (30 mL) at 0° C. The reaction mixture was stirred for 7 hrs at R.T, and then concentrated under reduced pressure to obtain a residue. A mixture of the residue and aq. HCl (6 N, 50 mL) was stirred overnight at 100° C., cooled to R.T, poured into ice/water, and then filtered. The filter cake was collected and dried to afford INT A5-2 (5.38 g, 72.97%). MS m/z: 365 [M−H]⁻.

DIEA (4.89 g, 37.84 mmol) was added dropwise to a solution of INT A5-2 (5.38 g, 14.66 mmol) in POCl₃ (16 mL) at 0° C. The reaction mixture was stirred for 2 hrs at 110° C. under an atmosphere of nitrogen, and then concentrated under reduced pressure to obtain a residue. Ice-water (100 mL) was added slowly to a mixture of the residue and DCM (100 mL); and the organic layer was washed with brine (2×100 mL), dried over Na₂SO₄ and concentrated under reduced pressure to obtain a residue. A solution of the residue and EA (400 mL) was washed with brine (3×200 mL), dried over Na₂SO₄ and then concentrated under reduced pressure to afford the INT A5 (5.33 g, 13.20 mmol) as a yellow solid. MS m/z: 403/405 [M+H]⁺.

### Intermediate A6 (INT A6)

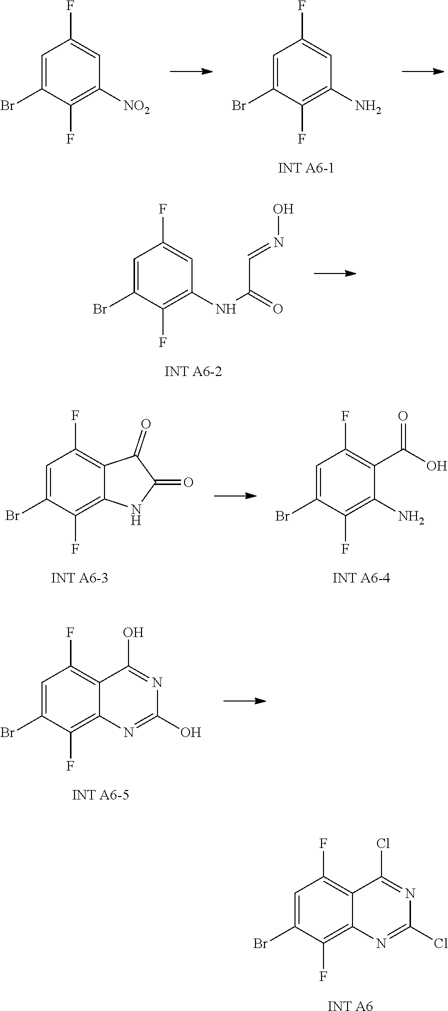

A mixture of 1-bromo-2,5-difluoro-3-nitrobenzene (3.11 g, 13.06 mmol), iron (2.12 g, 37.96 mmol), NH₄Cl (3.49 g, 65.24 mmol), ethanol (60 mL) and water (12 mL) was stirred at 80° C. for 2 hrs, and then filtered. The filtrate was concentrated under reduced pressure to obtain a residue. A solution of the residue in DCM (100 mL) was washed with brine (2×30 mL), dried over anhydrous Na₂SO₄ and concentrated under reduced pressure to afford INT A6-1 (2.49 g, 11.97 mmol, 91.6% yield). MS (ESI, m/z): 208 [M+H]⁺.

Concentrated hydrochloric acid (1.75 mL) was added to a mixture of INT A6-1 (2.49 g, 11.97 mmol), hydroxylammoniumchlorid (2.49 g, 35.83 mmol), Na₂SO₄ (11.64 g, 95.76 mmol), chloral hydrate (2.56 g, 17.95 mmol), water (50 mL) and ethanol (7 mL). The reaction mixture was stirred at 60° C. for 16 hrs, cooled to R.T, and then filtered. The filter cake was dried under vacuum to afford INT A6-2 (3.295 g, 11.80 mmol, 98.6% yield). MS (ESI, m/z): 279 [M+H]⁺.

INT A6-2 (3.295 g, 11.80 mmol) was added to sulfuric acid (29.5 mL) at 60° C. The reaction mixture was stirred at 90° C. for 1 h, cooled to R.T, and added slowly to ice/water to precipitate a solid. The solid was collected by filtration, washed with water and dried under vacuum to afford INT A6-3 (2.173 g, 8.29 mmol, 70.2% yield). MS (ESI, m/z): 262 [M+H]⁺.

Hydrogen dioxide (5.2 mL) was added dropwise to a solution of INT A6-3 (2.173 g, 8.29 mmol) in aq. NaOH (2 M, 46 mL, 93.50 mmol) at ° C. The reaction mixture was stirred at R.T for 16 hrs, quench with excessive sodium sulfite, and then the pH of the reaction mixture was adjusted to 7. The resulting mixture was filtered, and the pH of the filtrate was adjusted to 2 with concentrated hydrochloric acid to precipitate a solid. The solid was collected by filtration, washed with water and dried under vacuum to afford INT A6-4 (1.782 g, 7.07 mmol, 69.8% yield). MS (ESI, m/z): 252 [M+H]⁺.

Chlorosulfonyl isocyanate (1.33 g, 9.39 mmol) was added dropwise to a solution of INT A6-4 (1.782 g, 7.07 mmol) in dichloromethane (20 mL) at 0° C. The reaction mixture was stirred at R.T for 6 hrs and concentrated under reduced pressure to obtain a residue. A mixture of the residue and concentrated hydrochloric acid (20 mL) was stirred at 100° C. for 16 hrs, cooled to R.T, and then filtered. The filter cake was washed by water and dried under vacuum to afford INT A6-5 (0.83 g, 2.99 mmol, 75.5% yield). MS (ESI, m/z): 277 [M+H]⁺.

N,N-diisopropylethylamine (2 mL) was added to a solution of INT A6-5 (0.83 g, 2.99 mmol) in POCl₃ (15 mL). The reaction mixture was stirred at 105° C. for 2 hrs, and then concentrated under reduced pressure to obtain a residue. A solution of residue in DCM (50 mL) was washed by water (2×30 mL), dried over anhydrous Na₂SO₄, and then concentrated under reduced pressure to afford INT A6 (1.87 g, 5.95 mmol, 113.8% yield).

### Intermediate A7 (INT A7)

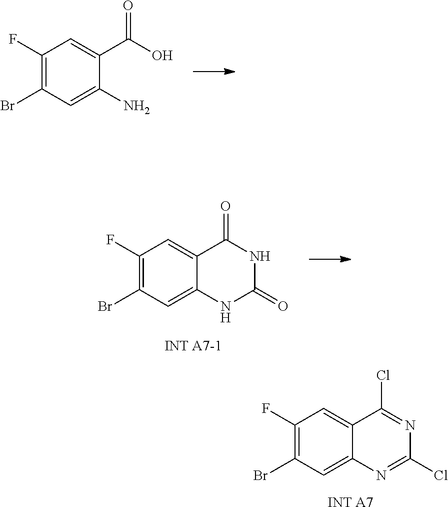

Chlorosulfonyl isocyanate (3.0 g, 21.00 mmol, 2.34 eq) was added dropwise over 15 mins to a solution of 2-amino-4-bromo-5-fluorobenzoic acid (2.1 g, 8.97 mmol, 1.0 eq) in DCM (35 mL) at 0° C. The reaction mixture was stirred for 48 hrs at R.T, and then concentrated to obtain a residue. A mixture of aq. hydrochloric acid (6 N, 70 mL) and the residue was stirred overnight at 105° C., cooled to R.T and then filtered to obtain a crude product containing INT A7-1 (1.15 g, 49.6% yield) as a white solid which was used for next step without any further purification. ¹H NMR (300 MHz, DMSO-d₆): δ 11.51 (s, 1H), 11.24 (s, 1H), 7.75 (d, J=8.4 Hz, 1H), 7.45 (d, J=5.7 Hz, 1H). LCMS: 257 ([M−H]⁻).

DIEA (8.7 g, 67.6 mmol, 5.0 eq) was added to a mixture of the crude product containing INT A7-1 (3.5 g, 13.5 mmol, 1.0 eq) and POCl₃ (35 mL) at R.T. The reaction mixture was concentrated after stirring overnight at 80° C., and then poured into ice/water to precipitate a solid. The solid was collected by filtration, and washed with water, dried to obtain a crude product containing INT A7 (2.7 g) as a yellow solid which was used for next step without any further purification. ¹H NMR (300 MHz, DMSO-d₆): δ 8.61 (d, J=6.6 Hz, 1H), 8.28 (d, J=8.4 Hz, 1H). LCMS: 295 ([M+H]⁺).

### Intermediate A8 (INT A8)

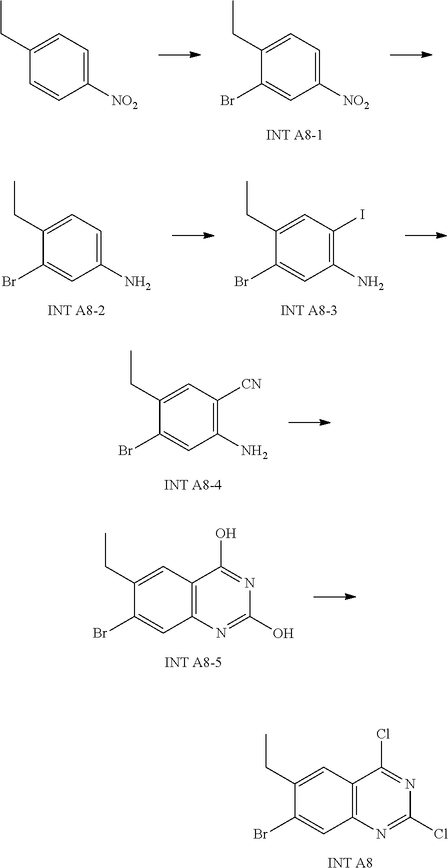

1-ethyl-4-nitrobenzene (10.0 g, 66 mmol, 1.0 eq), Ag₂SO₄ (21.0 g, 66 mmol, 1.0 eq), and Br₂ (11.0 g, 66 mmol, 1.0 eq) were added to a mixture of concentrated sulfuric acid (60 mL) and H₂O (7 mL). The reaction mixture was stirred at R.T for 1 h, and aq. sodium sulfite solution (10%, 100 mL) was added after the reaction was completed. The resulting mixture was extracted with EtOAc (100 mL×3). The organic layers were combined, dried over Na₂SO₄, and concentrated to afford a crude product containing INT A8-1 (16 g) as a brown solid which was used directly for next step without any further purification.

Fe powder (12.0 g, 217.3 mmol, 5.0 eq) was added to a mixture of the crude product containing INT A8-1 (10.0 g), MeOH (25 mL) and AcOH (25 mL). The reaction mixture was stirred at R.T for 1 h, and then filtered after the reaction was completed. Ammonia water (105 mL) was added to the filtrate, and the resulting mixture was extracted with EtOAc (150 mL×3). The organic layers were combined, dried over Na₂SO₄, and concentrated to obtain a crude product containing INT A8-2 (9.8 g) as a brown solid which was used directly for next step without any further purification. LCMS: 200.0 ([M+H]⁺).

NIS (5.87 g, 26.0 mmol, 0.9 eq) was added to a solution of the crude product containing INT A8-2 (5.8 g) in AcOH (58 mL). The reaction mixture was stirred at R.T for 1 h, and then aq. Na₂SO₄ (1 M, 50 mL) was added. The resulting mixture was extracted with EtOAc (50 mL×3). The organic layers were combined, dried over Na₂SO₄ and then concentrated to obtain a residue which was purified with silica gel chromatography (petroleum ether/ethyl acetate=50:1) to afford INT A8-3 (3.7 g, 11.4 mmol, 39.3% yield) as a yellow solid. LCMS: 325.9 ([M+H]⁺).

Zn(CN)₂ (645.8 mg, 5.5 mmol, 0.5 eq) and Pd(pph₃)₄ (638.1 mg, 0.55 mmol, 0.05 eq) were added to a solution of INT A8-3 (3.6 g, 11.0 mmol, 1.0 eq) in DMF (50 mL). The reaction mixture was stirred at 90° C. for 4.5 hrs under N₂ atmosphere, diluted with H₂O (60 mL) after the reaction was completed, and then extracted with EtOAc (60 mL×3). The organic layers were combined, dried over Na₂SO₄, and concentrated to obtain a crude product containing INT A8-4 (2.9 g) as a yellow solid which was used directly for next step without any further purification. LCMS: 225.0 ([M+H]⁺).

A solution of the crude product containing INT A8-4 (1.0 g) in DBU-HOCH₃CF₃ (10 ml) was stirred overnight at 25° C. under an atmosphere of CO₂. The resulting mixture was diluted with H₂O (20 ml), and extracted with EtOAc (20 mL×3). The organic layers were combined, dried over Na₂SO₄ and concentrated to obtain a residue which was triturated and slurried with a mixed solution of DCM and MeOH (V_{DCM}/V_{MeOH}=10:1) to afford INT A8-5 (410 mg, 1.5 mmol, 38% yield) as a white solid. ¹H NMR (300 MHz, DMSO-d₆): δ 11.23 (s, 2H), 7.78 (s, 1H), 7.39 (s, 1H), 2.71 (q, J=7.4 Hz, 2H), 1.17 (t, J=7.5 Hz, 3H). LCMS: 269.0 ([M+H]⁺).

DIEA (0.75 ml) was dropwise added to a solution of INT A8-5 (410 mg, 1.5 mmol, 1.0 eq) in POCl₃ (7.5 mL). The reaction mixture was stirred at 107° C. for 2 hrs, and then concentrated to afford a crude product containing INT A8 as a black solid which was used directly for next step without any further purification. LCMS: 304.9 ([M+H]⁺).

### Intermediate A9 (INT A9)

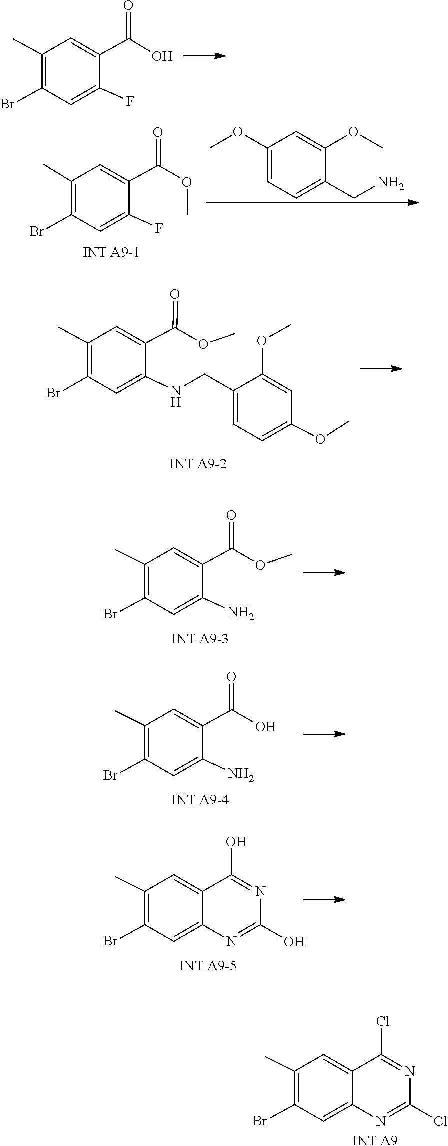

MeI (5.3 g, 37.5 mmol, 1.5 eq) and K₂CO₃ (10.4 g, 75.2 mmol, 3.0 eq) were added to a solution of 4-bromo-2-fluoro-5-methylbenzoic acid (5.8 g, 24.9 mmol, 1.0 eq) in DMF (60 mL) below 10° C. The reaction mixture was stirred for 30 mins at R.T, poured into water (100 mL) and extracted with EtOAc (100 mL×2). The organic layers were combined, washed with water (75 mL×2) and brine (50 mL×2) successively, dried over anhydrous Na₂SO₄, and concentrated to obtain a residue which was slurried with hexane and filtered to afford INT A9-1 (5.4 g, yield 83.3%) as a white solid. ¹H NMR (300 MHz, CDCl₃): δ 7.79 (d, J=7.4 Hz, 1H), 7.36 (d, J=9.9 Hz, 1H), 3.92 (s, 3H), 2.39 (s, 3H). LCMS: 247.0, 249.0 ([M+H]⁺).

2,4-Dimethoxybenzylamine (5.2 g, 30.6 mmol, 1.2 eq) and K₂CO₃ (10.6 g, 76.5 mmol, 3.0 eq) were added to a solution of INT A9-1 (6.3 g, 25.5 mmol, 1.0 eq) in 1,4-dioxane (60 mL) at R.T. The reaction mixture was stirred for 3 hrs at 100° C., cooled to room temperature, poured into ice-water (20 mL) and extracted with EtOAc (20 mL×3). The organic layers were combined, washed with brine (20 mL), dried with anhydrous Na₂SO₄, and concentrated to afford the residue containing INT A9-2 which was used for next step without further purification. ¹H NMR (300 MHz, DMSO-d₆): δ 7.83 (t, J=6.0 Hz, 1H), 7.65 (d, J=0.42, 1H), 7.15 (d, J=8.1 Hz, 1H), 6.99 (s, 1H), 6.61-6.59 (m, 1H), 6.51-6.47 (m, 1H), 4.28 (d, J=6.0 Hz, 2H), 3.83 (s, 3H), 3.79 (s, 3H), 3.75 (s, 3H), 2.21 (s, 3H).

A mixture of the residue containing INT A9-2 (7.4 g, 21.4 mmol, 1.0 eq), TFA (3 mL) and DCM (15 mL) was stirred at the R.T for 3 hrs, and then concentrated to afford a crude product containing INT A9-3 (5.4 g) which was used for next step without further purification. LCMS: 244.2, 246.2 ([M+H]⁺).

NaOH (1.9 g, 46.2 mmol, 3.0 eq) was added to a solution of the crude product containing INT A9-3 (4.6 g, 15.4 mmol, 1.0 eq) in a mixed solution of THF, EtOH, and water (V_{THF}/V_{EtOH}/V_{water}=3:1:1, 50 mL) at R.T. The reaction mixture was stirred for 2.5 hrs at 50° C., then concentrated, diluted with water (20 mL) and extracted with EtOAc (40 mL). The organic layer was washed with brine (20 mL), dried with anhydrous Na₂SO₄, and concentrated to obtain a residue which was purified with silica gel chromatography (eluting with petroleum ether/EtOAc=30:1) to afford a crude product containing INT A9-4 (5.2 g) as a white solid which was used for next step without further purification. LCMS: 227.8, 230.0 ([M−H]⁻).

Chlorosulfonyl isocyanate (7.3 g, 51.6 mmol, 2.4 eq) was added dropwise to a solution of the crude product containing INT A9-4 (5.0 g, 21.5 mmol, 1.0 eq) in DCM (50 mL). The reaction mixture was stirred at room temperature for 1 h, and then concentrated to obtain a residue. A mixture of the residue and aq. hydrochloric acid (6 N, 100 mL) was stirred for 16 hrs at 100° C., cooled to R.T and filtered to afford a solid containing INT A9-5 (4.5 g, yield 82.0%) as a white solid which was used for next step without further purification. ¹H NMR (300 MHz, DMSO-d₆): δ 11.33 (brs, 1H), 11.12 (brs, 1H), 7.83 (s, 1H), 7.38 (s, 1H), 2.36 (s, 3H). LCMS: 252.8, 254.8 ([M−H]⁻).

DIEA (646.0 mg, 5.0 mmol, 5.0 eq) was added to a solution of the solid containing INT A9-5 (1.0 g, 3.9 mmol, 1.0 eq) in POCl₃ (20 mL) at R.T The reaction mixture was heated to reflux and stirred for 16 hrs, and then concentrated under reduced pressure to obtain a residue which was purified with silica gel chromatography (eluting with petroleum ether/EtOAc=100:1) to afford INT A9 (400.0 mg, yield 35.1%) as a white solid. LCMS: 292.9 ([M+H]⁺).

### Intermediate A10 (INT A10)

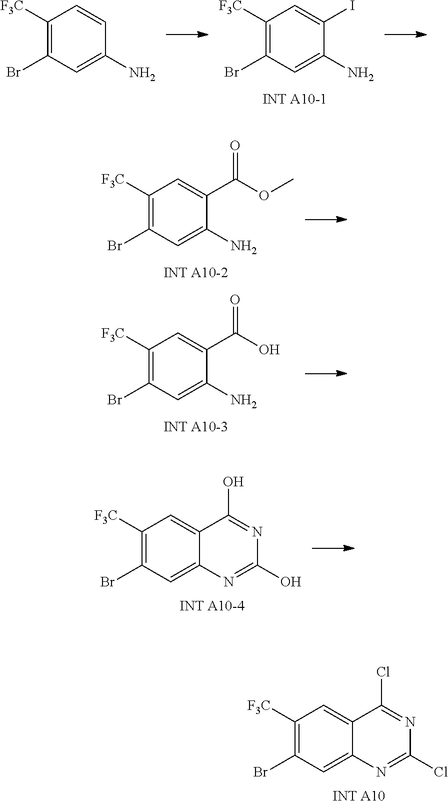

NIS (5.6 g, 25.0 mmol, 1.0 eq) was added in batches to a solution of 3-bromo-4-(trifluoromethyl)aniline (6.0 g, 25.0 mmol, 1.0 eq) in AcOH (60 mL) when the temperature was below 10° C. The reaction mixture was stirred at R.T for 5 hrs, and then concentrated to obtain a residue which was purified with silica gel chromatography (eluting with petroleum ether) to afford INT A10-1 (7.0 g, yield 85.9%) as a white solid. ¹H NMR (300 MHz, DMSO-d₆): δ 7.82 (s, 1H), 7.07 (s, 1H), 6.19 (s, 2H). LCMS: 363.8, 365.8 ([M−H]⁻).

INT A10-1 (3.1 g, 8.5 mmol, 1.0 eq), Pd(dppf)Cl₂-DCM (694 mg, 0.85 mmol, 0.1 eq), TEA (6.0 g, 59.5 mmol, 7.0 eq) and MeOH (30 mL) were placed in a 300 mL stainless steel autoclave. The autoclave was sealed and the reaction mixture was stirred at 30° C. under an atmosphere of carbon monoxide (0.8 MPa) for 13 hrs. After the reaction was completed, the resulting mixture was concentrated to obtain a residue which was purified with silica gel chromatography (eluting with petroleum ether/EtOAc=100:1) to afford INT A10-2 (1.8 g, yield 71.1%) as a white solid. ¹H NMR (300 MHz, CDCl₃): δ 8.15 (s, 1H), 6.99 (s, 1H), 6.11 (brs, 2H), 3.89 (s, 3H). LCMS: 297.8, 299.8 ([M+H]⁺).

NaOH (720.0 mg, 18.0 mmol, 3.0 eq) was added to a solution of INT A10-2 (1.8 g, 6.0 mmol, 1.0 eq) in a mixed solution of THF, EtOH and water (V_{THF}/V_{EtOH}/V_{water}=3:1:1, 40 mL) at room temperature. The reaction mixture was stirred for 3 hrs at 50° C., and then diluted with water (20 mL). The pH of the resulting mixture was adjusted to 2 with 6 N HCl, and then EtOAc (40 mL) was added. The organic layer was washed with brine (20 mL), dried with anhydrous Na₂SO₄, and concentrated to afford a crude product containing INT A10-3 (1.6 g, 93.0%) which was used for next step without further purification. LCMS: 282.0, 283.9 ([M−H]⁻).

Chlorosulfonyl isocyanate (1.9 g, 13.5 mmol, 2.4 eq) was added dropwise to a solution of the crude product containing INT A10-3 (1.6 g) in DCM (20 mL). The reaction mixture was stirred at R.T for 2 hrs, and then concentrated to obtain a residue. A mixture of the residue and aq. HCl (6 N, 100 mL) was stirred for 16 hrs at 100° C., cooled to R.T and filtered to afford a crude product containing INT A10-4 (1.2 g, 69.0%) as a white solid which was used for next step without further purification. LCMS: 306.8, 308.8 ([M−H]⁻).

DIEA (1.9 g, 15.0 mmol, 5.0 eq) was added to a mixture of the crude product containing INT A10-4 (900.0 mg, 3.0 mmol, 1.0 eq) and POCl₃ (20 mL) at R.T. The reaction mixture was heated to refluxed and stirred for 16 hrs, and then concentrated under reduced pressure to obtain a residue which was purified with silica gel chromatography (eluting with petroleum ether/EtOAc=100:1) to afford a crude product containing INT A10 (330 mg) as a yellow solid which was used for next step without further purification.

### Intermediate A11 (INT A11)

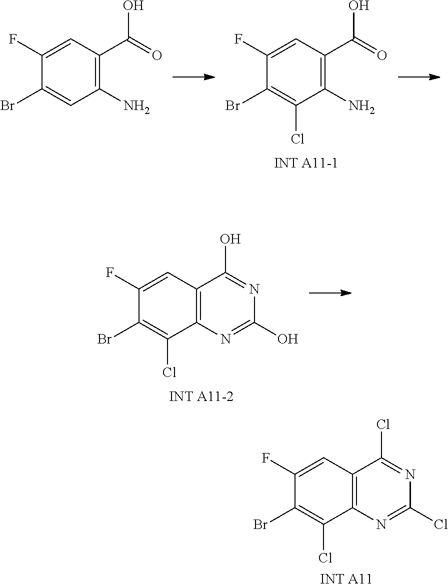

NCS (1.7 g, 13.0 mmol, 1.0 eq) was added in batches to a solution of 2-amino-4-bromo-5-fluorobenzoic acid (3.1 g, 13.0 mmol, 1.0 eq) in DMF (124.0 mL) at R.T. The reaction mixture was stirred overnight, diluted with water (300 mL) after the reaction was completed, extracted with EtOAc (3×300 mL). The organic layers were combined, washed with brine and dried over anhydrous Na₂SO₄, concentrated to afford a crude product containing INT A11-1 (2.4 g) which was used directly to the next step without further purification. ¹H NMR (400 MHz, DMSO-d₆) δ 7.64 (d, J=9.4 Hz, 1H). LCMS: 265.9 ([M−H]⁻).

Chlorosulfonyl isocyanate (2.5 g, 17.5 mmol, 2.3 eq) was added slowly to a solution of the crude product containing INT A11-1 (2.0 g) in DCM (46 mL) at 0° C. The reaction mixture was stirred at R.T for 4 hrs, and then concentrated to afford a residue. A mixture of the residue and aq. HCl (6 N, 54 mL) was heated to reflux, stirred overnight, and then cooled to precipitate the solid. The solid was collected by filtration, dried to afford a crude product containing INT A11-2 (2.1 g) as a yellow solid which was used directly to the next step without further purification. ¹H NMR (300 MHz, DMSO-d₆) δ 11.72 (s, 1H), 10.87 (s, 1H), 7.78 (d, J=8.0 Hz, 1H). LCMS: 290.9 ([M−H]⁻).

DIEA (1.7 mL) was added to a mixture of the crude product containing INT A11-2 (1.6 g) and POCl₃ (17 mL) at R.T. The reaction mixture was stirred at 110° C. for 5 hrs, and then concentrated to afford a crude product containing INT A11 which was used directly to the next step without any further purification.

### Intermediate A12 (INT A12)

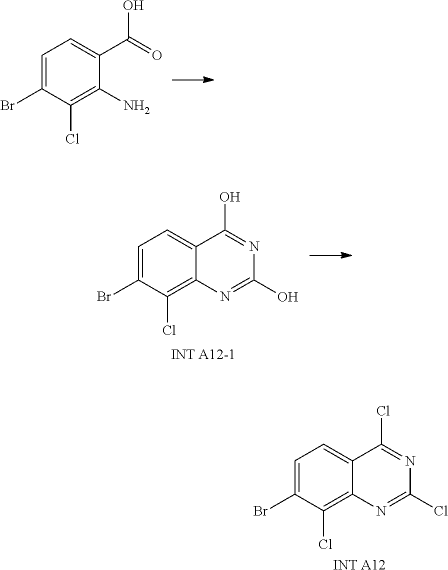

INT A12 was synthesized following the procedure of INT A3 with 2-amino-4-bromo-3-chlorobenzoic acid as starting material.

### Intermediate A13 (INT A13)

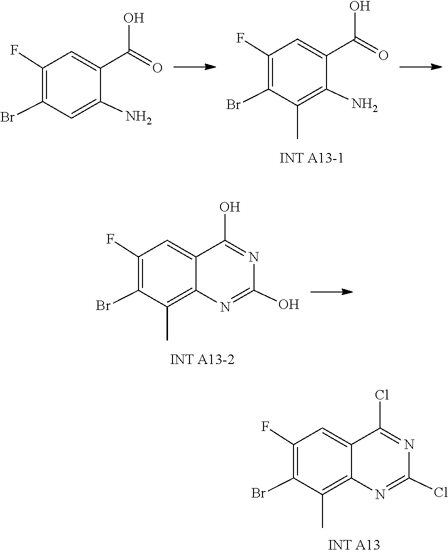

INT A13 was synthesized following the procedure of INT A2 with 2-amino-4-bromo-5-fluorobenzoic acid as starting material.

### Intermediate B1 (INT B1)

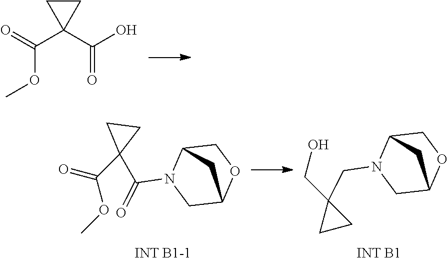

To a solution of 1-(methoxycarbonyl)cyclopropane-1-carboxylic acid (15.18 g, 105.32 mmol) in DCM (200 mL) was added DMF (0.15 mL, 1.93 mmol). The mixture was cooled to 0° C. and then oxalyl chloride (19.50 g, 153.63 mmol) was added dropwise. The reaction mixture was stirred at room temperature for 2 hrs and concentrated under vacuum to obtain a residue. A solution of the residue dissolved in DCM (50 mL) was added dropwise to a solution of (1S,4S)-2-oxa-5-azabicyclo[2.2.1]heptane hydrochloride (19.90 g, 146.76 mmol) and TEA (50 mL, 359.72 mmol) in DCM (200 mL). After the reaction was completed, the resulting mixture was quenched with water (100 mL), and the organic layer was dried over Na₂SO₄ and concentrated under vacuum to afford INT B1-1 (24.93 g, 110.68 mmol, 105.0% yield). MS m/z: 226 [M+H]⁺.

To a solution of INT B1-1 (24.93 g, 110.68 mmol) in THF (200 mL) was added dropwise LAH (200 mL, 110.68 mmol, 1 M in THF) at 0° C. The reaction mixture was stirred at room temperature for 2 hrs; quenched with water (8 mL), aq. NaOH (15%, 8 mL), and water (24 mL) successively; and then filtered. The filtrate was concentrated under reduced pressure and the residue was purified with silica gel column chromatography (eluting with 0-40% EA in Hex) to afford INT B1 (17.15 g, 93.58 mmol, 84.5% yield). MS m/z: 184 [M+H]⁺.

The following intermediates were synthesized following the procedure of INT B1 with corresponding starting material:

| No. | Structure |
|---|---|
| INT B2 | 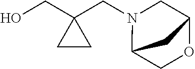 [SMILES: OCC1(CN2C[C@H]3C[C@@H]2CO3)CC1] |
| INT B3 | 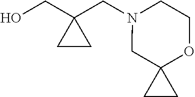 [SMILES: OCC1(CN2CCOC3(CC3)C2)CC1] |
| INT B4 | 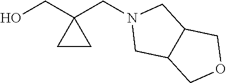 [SMILES: OCC1(CN2CC3COCC3C2)CC1] |
| INT B5 | 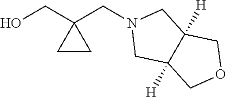 [SMILES: OCC1(CN2C[C@H]3COC[C@H]3C2)CC1] |
| INT B6 | 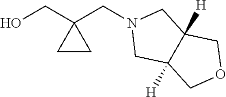 [SMILES: OCC1(CN2C[C@@H]3COC[C@H]3C2)CC1] |
| INT B7 | 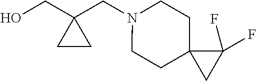 [SMILES: OCC1(CN2CCC3(CC2)CC3(F)F)CC1] |
| INT B8 | 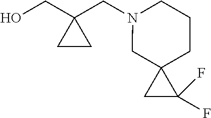 [SMILES: OCC1(CN2CCCC3(C2)CC3(F)F)CC1] |
| INT B9 | 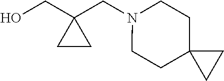 [SMILES: OCC1(CN2CCC3(CC2)CC3)CC1] |
| INT B10 | 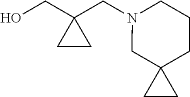 [SMILES: OCC1(CN2CCCC3(CC3)C2)CC1] |
| INT B11 | 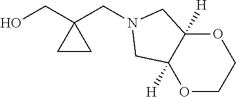 [SMILES: OCC1(CN2C[C@@H]3OCCO[C@@H]3C2)CC1] |
| INT B12 | 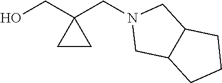 [SMILES: OCC1(CN2CC3CCCC3C2)CC1] |
| INT B13 | 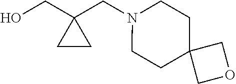 [SMILES: OCC1(CN2CCC3(CC2)COC3)CC1] |
| INT B14 | 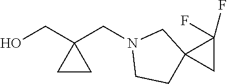 [SMILES: OCC1(CN2CCC3(C2)CC3(F)F)CC1] |
| INT B15 | 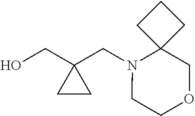 [SMILES: OCC1(CN2CCOCC23CCC3)CC1] |
| INT B16 | 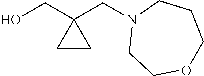 [SMILES: OCC1(CN2CCCOCC2)CC1] |

### Intermediate B17 (INT B17)

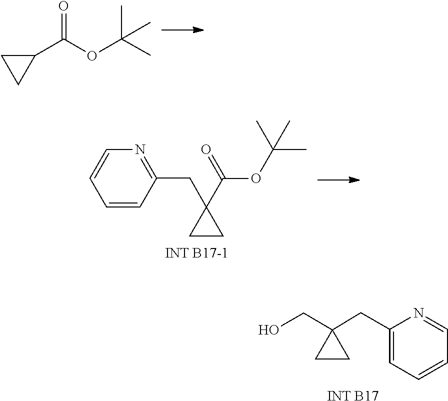

A solution of tert-butyl cyclopropanecarboxylate (5.0 g, 35.2 mmol, 1.0 eq) in THF (120 mL) was purged with nitrogen for 10 mins, and then LDA (2.0 M, 19 mL, 1.1 eq) was added dropwise at −78° C. under an atmosphere of nitrogen. The mixture was stirred for 1 h at −78° C., and maintaining this temperature, a solution of 2-(bromomethyl) pyridine (6.1 g, 56.3 mmol, 1.6 eq) in THF (30 mL) was added dropwise. The resulting mixture was warmed to R.T, stirred for 16 hrs, quenched with saturated NH₄Cl (50 mL), diluted with water (100 mL) and extracted with EtOAc (100 mL×2). The organic layers were combined, washed with brine (100 mL), dried over Na₂SO₄, and then filtered. The filtrate was concentrated to obtain a residue which was purified with silica gel chromatography (eluting with petroleum ether/EtOAc=10:1) to afford INT B17-1 (2.2 g) as a white solid. LCMS: 234.2 ([M+H]⁺).

LAH (1.8 g, 47.1 mmol, 5.0 eq) was added to a solution of INT B17-1 (2.2 g, 9.4 mmol, 1.0 eq) in THF (20 mL) at 0° C. The mixture was stirred at R.T for 2 hrs, cooled to 0° C., and then Na₂SO₄·10H₂O (4.5 g, 14.1 mmol, 1.5 eq) was added in batches. The reaction mixture was stirred at R.T for 2 hrs and then filtered. The filtrate was concentrated to obtain a residue which was purified with silica gel chromatography (eluting with petroleum ether/EtOAc=2:1) to afford INT B17 (800.0 mg, the yield of the above two steps is 14.0%) as a yellow oil. ¹H NMR (300 MHz, CDCl₃): δ 8.52-8.50 (m, 1H), 7.61 (td, J=7.5, 1.8 Hz, 1H), 7.18-7.10 (m, 2H), 3.44 (s, 2H), 2.94 (s, 2H), 0.56-0.53 (m, 2H), 0.46-0.44 (m, 2H). LCMS: 164.20 ([M+H]⁺).

### Intermediate C1 (INT C1)

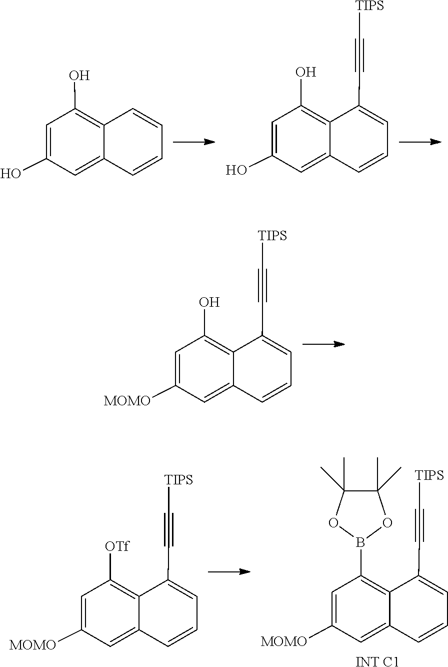

Following the procedure of WO2021041671, INT C1 (Intermediate C1) was synthesized with naphthalene-1,3-diol as starting material.

### Intermediate C2 (INT C2)

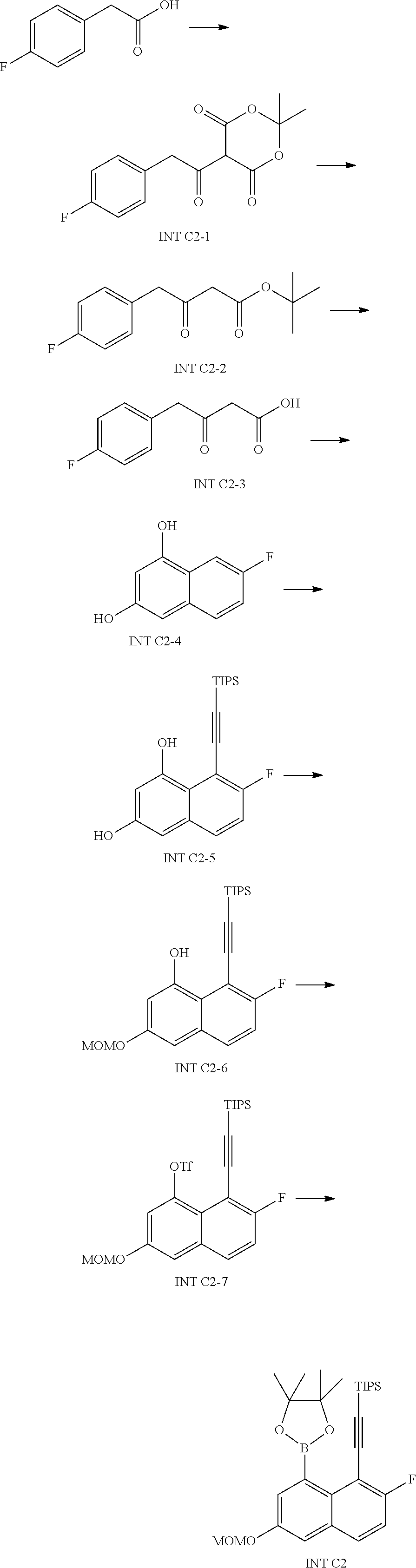

Following the procedure of WO2021041671, INT C2 was synthesized with 2-(4-fluorophenyl) acetic acid as starting material.

### Intermediate C3 (INT C3)

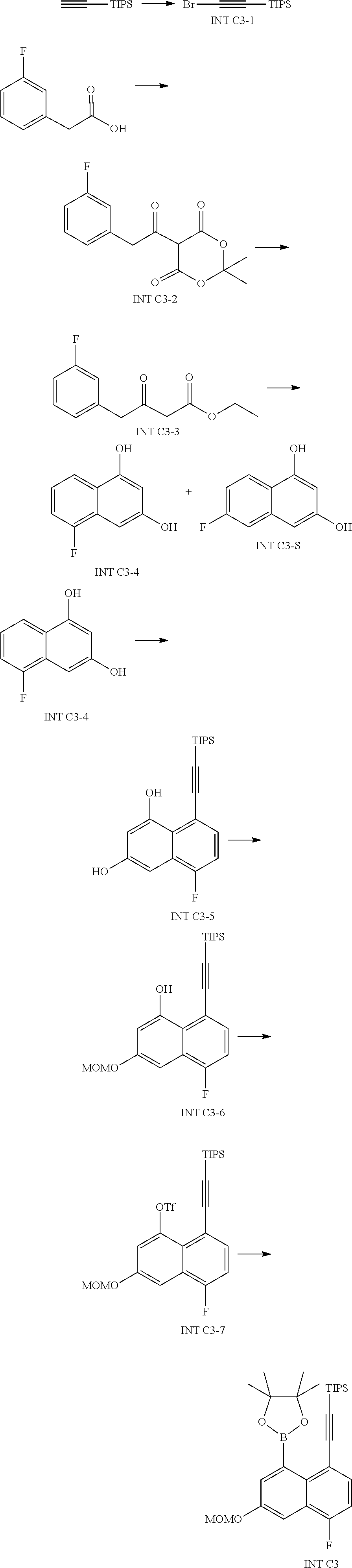

Following the procedure of WO2021041671, INT C3-1 was synthesized with ethynyltriisopropylsilane as starting material.

Maintaining the temperature below 45° C., DIEA (90.1 g, 697.4 mmol, 2.2 eq) was added to a solution of 2-(3-fluorophenyl)acetic acid (50.0 g, 324.4 mmol, 1.0 eq), 2,2-Dimethyl-1,3-dioxane-4,6-dione (51.4.0 g, 356.8 mmol, 1.1 eq) and DMAP (3.4 g, 27.6 mmol, 0.09 eq) in MeCN (150 mL), and then pivaloyl chloride (43.0 g, 356.8 mmol, 1.1 eq) was slowly added over 3 hrs to the mixture. The reaction mixture was stirred at 45° C. for 3 hrs, cooled to 0° C., and then aq. HCl (1 N, 500 mL) was slowly added. The resulting solution was stirred at 0° C. for 2 hrs and a solid was precipitated. The solid was collected by filtration and dried to afford a crude product which was washed with diethyl ether (300 mL) to obtain INT C3-2 (89.0 g, 97.9%) as a white solid. ¹H NMR (300 MHz, CDCl₃): δ 15.34 (s, 1H), 7.30-7.15 (m, 4H), 4.41 (s, 2H), 1.72 (s, 6H). LCMS: 279.1 ([M−H]⁻).

A solution of INT C3-2 (25.0 g, 89.2 mmol, 1.0 eq) in EtOH (75 mL) was stirred at 90° C. for 2 hrs, and then concentrated to obtain a crude containing INT C3-3 (20.0 g, 99.0%) as a yellow oil. LCMS: 225.1 ([M+H]⁺).

INT C3-3 (20.0 g, 89.2 mmol, 1.0 eq) was added in batches to concentrated H₂SO₄ (65.5 g, 668.6 mmol, 7.5 eq) at 0° C. The reaction mixture was stirred at room temperature for 24 hrs, cooled to 0° C. and then poured slowly into ice-water (300 mL). The resulting mixture was filtered, and the filter cake was dispersed in petroleum ether (100 mL). The solid was collected by filtration and dried to afford INT C3-4 (5.0 g) and INT C3-S (7.7 g, 48.5%).

INT C3-S ¹H NMR (400 MHz, DMSO-d₆): δ 10.22 (s, 1H), 9.63 (s, 1H), 7.97 (t, J=2.8 Hz, 1H), 7.32-7.29 (m, 1H), 7.03-6.98 (m, 1H), 6.57 (d, J=2.0 Hz, 1H), 6.45 (d, J=2.0 Hz, 1H). LCMS: 177.1 ([M−H]⁻).

A mixture of INT C3-4 (5.0 g, 28.1 mmol, 1.0 eq), dichloro(p-cymene) ruthenium(II) dimer (1.7 g, 2.8 mmol, 0.1 eq), AcOK (5.5 g, 56 mmol, 2.0 eq) and dioxane (35 mL) was purged with N₂ for three times, heated to 110° C. for 1.5 hrs, cooled to room temperature, and then filtered. The filtrate was concentrated to obtain a residue which was purified by silica gel chromatography (petroleum ether/EtOAc=30:1 to 10:1) to afford INT C3-5 (10.1 g, 28.2 mmol, 98%) as a black oil. ¹H NMR (300 MHz, DMSO-d₆): δ 10.26 (s, 1H), 9.92 (s, 1H), 7.29 (dd, J=8.0, 5.7 Hz, 1H), 7.12 (dd, J=10.7, 8.0 Hz, 1H), 6.69 (d, J=1.7 Hz, 1H), 6.60 (d, J=2.3 Hz, 1H), 1.12 (s, 21H). LCMS: 357.2 ([M−H]⁻).

MOMCl (1.3 g, 16.4 mmol, 1.3 eq) was added dropwise to a mixture of INT C3-5 (4.5 g, 12.6 mmol, 1.0 eq), DIEA (4.9 g, 37.8 mmol, 3.0 eq) and DCM (120 mL) at 0° C. The reaction mixture was stirred at 0° C. for 3 hrs, poured into ice water (100 mL), and then extracted with DCM (100 mL×2). The organic layers were combined, washed with brine (100 mL), dried over Na₂SO₄, filtered and then concentrated to afford a residue which was purified by silica gel chromatography (petroleum ether/EtOAc=80:1) to obtain INT C3-6 (3.1 g, 7.7 mmol, 29.6%) as a yellow oil. ¹H NMR (300 MHz, CDCl₃): δ 9.31 (s, 1H), 7.42 (dd, J=8.0, 5.5 Hz, 1H), 7.17 (d, J=2.4 Hz, 1H), 7.02 (dd, J=10.1, 8.0 Hz, 1H), 6.81 (d, J=2.4 Hz, 1H), 5.28 (s, 2H), 3.51 (s, 3H), 1.18 (s, 21H). LCMS: 401.2 ([M−H]⁻).

Tf₂O (2.1 g, 7.4 mmol, 1.5 eq) was added dropwise to a solution of INT C3-6 (2.0 g, 4.9 mmol, 1.0 eq) and DIEA (1.9 g, 14.9 mmol, 3.0 eq) in DCM (30 mL) at −40° C. The reaction mixture was stirred at −40° C. for 30 mins, quenched with water (80 mL), and the aqueous layer was extracted with DCM (50 mL×2). The organic layers were combined, washed with brine, dried over Na₂SO₄, filtered and then concentrated to obtain a residue which was purified with silica gel chromatography (petroleum ether/EtOAc=50:1 to 10:1) to afford INT C3-7 (3.8 g, 7.1 mmol, 93.5%) as a yellow oil. ¹H NMR (300 MHz, CDCl₃): δ 7.74-7.61 (m, 2H), 7.36 (d, J=2.3 Hz, 1H), 7.15 (dd, J=9.8, 8.2 Hz, 1H), 5.31 (s, 2H), 3.52 (s, 3H), 1.21-1.12 (m, 21H).

A mixture of INT C3-7 (3.0 g, 5.6 mmol, 1.0 eq), Bis(pinacolato)diborane (2.1 g, 8.4 mmol, 1.5 eq), Pd(dppf)Cl₂₋CH₂Cl₂ (228 mg, 0.28 mmol, 0.05 eq), dppf (155.2 mg, 0.28 mmol, 0.05 eq), KOAc (1.7 g, 16.9 mmol, 3.02 eq) and dioxane (22 mL) was stirred overnight at 110° C. under an atmosphere of N₂, cooled to room temperature, filtered and then concentrated under reduced pressure to obtain a residue. The residue was purified with silica gel chromatography (petroleum ether/EtOAc=80:1) to afford INT C3 (670 mg, 1.3 mmol, 23.3%) as a yellow solid. ¹H NMR (300 MHz, DMSO-d₆): δ 7.63-7.59 (m, 2H), 7.50 (d, J=2.4 Hz, 1H), 7.08-7.02 (m, 1H), 5.31 (s, 2H), 3.50 (s, 3H), 1.40 (s, 12H), 1.14 (s, 21H). LCMS: 513.0 ([M+H]⁺).

### Intermediate C4 (INT C4)

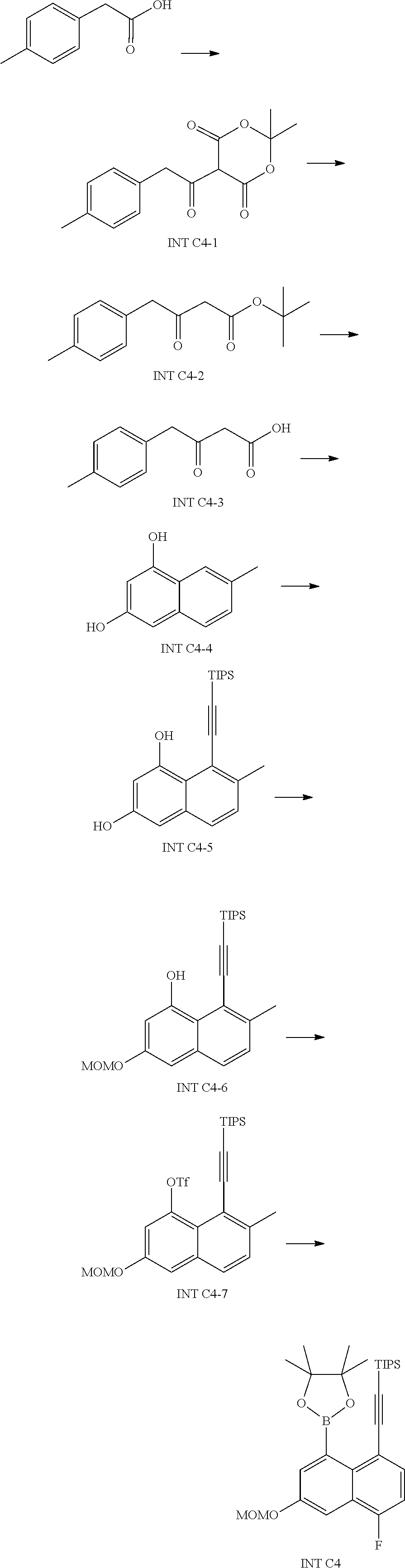

Maintaining temperature below 40° C., DIEA (46.3 g, 357.9 mmol, 2.15 eq.) and pivaloyl chloride (22.1 g, 183.1 mmol, 1.1 eq.) were slowly added successively to a mixture of 2-(p-tolyl)acetic acid (25.0 g, 166.5 mmol, 1.0 eq.), 2,2-dimethyl-1, 3-dioxane-4,6-dione (26.4 g, 183.1 mmol, 1.1 eq.), DMAP (1.7 g, 14.1 mmol, 0.085 eq.) and MeCN (75 mL). The reaction mixture was stirred at 45° C. for 3 hrs, cooled to 0° C., and then 1N HCI (250 mL) was slowly added to precipitate a solid. After stirring for 2 hrs at 0° C., the solid was collected by filtering, washed with diethyl ether (50 mL), and dried to afford INT C4-1 (41.6 g, 90.5%) as a yellow solid. ¹H NMR (300 MHz, DMSO-d₆): δ 7.22-7.12 (m, 4H), 4.32 (s, 2H), 2.28 (s, 3H), 1.69 (s, 6H). LCMS: 277 ([M+H]⁺).

A mixture of INT C4-1 (41.6 g, 150.6 mmol, 1.0 eq.) and t-BuOH (120 mL) was stirred at 90° C. for 2 hrs, and then concentrated to afford a crude containing the INT C4-2 (37.0 g, 98.9%) as a yellow oil which was used into next step without any further purification. ¹H NMR (300 MHz, DMSO-d₆): δ 7.15-7.05 (m, 4H), 3.79 (s, 2H), 3.51 (s, 2H), 2.32 (s, 3H), 1.43 (s, 9H). LCMS: 271.1 ([M+Na]⁺).

A solution of INT C4-2 (37.0 g, 192.5 mmol, 1.0 eq.) and TFA (149.5 g, 1.31 μmol, 6.81 eq.) in DCM (80 mL) was stirred at 20° C. for 1 h, and concentrated to obtain a crude product. The crude product was slurried with petroleum ether (40 mL) at room temperature for 1 h, and then filtered to afford INT C4-3 (23.0 g, 80.3%) as a white solid. ¹H NMR (300 MHz, DMSO-d₆): δ 7.14-7.06 (m, 4H), 3.81 (s, 2H), 3.48 (s, 2H), 2.28 (s, 3H). LCMS: 193.1 ([M+H]⁺).

A mixture of INT C4-3 (23.0 g, 119.7 mmol, 1.0 eq.) and CF₃SO₃H (449.0 g, 2.99 μmol, 25.0 eq.) was stirred overnight at 25° C., cooled to 0° C. and added slowly into ice/water (750 mL). The solid was collected by filtration, dried, slurred with petroleum ether (100 mL), and then filtered to afford INT C4-4 (19.0 g, 91.3%) as a yellow solid. ¹H NMR (400 MHz, DMSO-d₆): δ 9.93 (brs, 1H), 9.30 (brs, 1H), 7.73 (d, J=0.8 Hz, 1H), 7.46 (d, J=8.4 Hz, 1H), 7.18-7.16 (m, 1H), 6.53 (d, J=2.0 Hz, 1H), 6.46 (d, J=2.0 Hz, 1H), 2.39 (s, 3H). LCMS: 175.1 ([M+H]⁺).

KOAc (1.7 g, 17.2 mmol, 2.0 eq.), (bromoethynyl)triisopropylsilane (2.4 g, 9.0 mmol, 1.05 eq.), INT C4-4 (1.5 g, 8.6 mmol, 1.0 eq.), t-BuOH (10.5 mL, 7.0 V), and dichloro(p-cymene)ruthenium(II) dimer (2.6 g, 4.3 mmol, 0.5 eq.) were placed in a sealed vial. The reaction mixture was irradiated in the microwave at 160° C. for 2 hrs under the an atmosphere of N₂, cooled to R.T and filtered through a pad of celite. The filtrate was concentrated to afford a crude product which was purified with silica gel chromatography (petroleum ether/EtOAc=10:1) to afford INT C4-5 (430.3 mg, 14.1%) as a yellow oil. ¹H NMR (300 MHz, DMSO-d₆): δ 9.79 (s, 1H), 9.49 (s, 1H), 7.51 (d, J=8.4 Hz, 1H), 7.26 (d, J=8.4 Hz, 1H), 6.58-6.52 (m, 2H), 2.50 (s, 3H), 1.15-1.05 (m, 21H). LCMS: 355.2 ([M+H]⁺).

MOMCl (295.2 mg, 3.7 mmol, 1.3 eq.) was added dropwise to a mixture of INT C4-5 (1.0 g, 2.8 mmol, 1.0 eq.), DIEA (1.1 g, 8.5 mmol, 3.0 eq.) and DCM (10 mL) at 0° C. The reaction mixture was stirred overnight at R.T, and then poured into ice/water (10 mL) after the reaction was completed. The resulting mixture was extracted with DCM (10 mL×2). The organic layers were combined, washed with brine (10 mL), dried over Na₂SO₄, and concentrated to afford a residue. The residue was purified with silica gel chromatography (eluting with petroleum ether) to afford INT C4-6 (500.0 mg, 44.6%) as a yellow solid. ¹H NMR (300 MHz, DMSO-d₆): δ 10.01 (s, 1H), 7.64 (d, J=8.4 Hz, 1H), 7.35 (d, J=8.4 Hz, 1H), 6.88 (d, J=2.4 Hz, 1H), 6.66 (d, J=2.4 Hz, 1H), 5.23 (s, 2H), 3.40 (s, 3H), 2.52 (s, 3H), 1.15 (s, 21H).

A mixture of INT C4-6 (500.0 mg, 1.3 mmol, 1.0 eq.), DIEA (504.0 mg, 3.9 mmol, 3.0 eq.) and DCM (8 mL) was cooled to −40° C., then Tf₂O (550.2 g, 2.0 mmol, 1.5 eq.) was added. The reaction mixture was stirred at −40° C. for 30 mins and then quenched with water (5 mL) after the reaction was completed. The resulting mixture was extracted with DCM (5 mL×2). The organic layers were combined, washed with brine (5 mL×1), dried over Na₂SO₄, and concentrated to obtain a crude product which was purified with silica gel chromatography (petroleum ether/EtOAc=30:1) to afford INT C4-7 (501.0 mg, 72.6%) as a yellow oil. ¹H NMR (300 MHz, DMSO-d₆): δ 7.91 (d, J=8.4 Hz, 1H), 7.65 (d, J=2.4 Hz, 1H), 7.57 (d, J=8.4 Hz, 1H), 7.38 (d, J=2.1 Hz, 1H), 5.34 (s, 2H), 3.41 (s, 3H), 2.59 (s, 3H), 1.14 (s, 21H).

Pd(dppf)Cl₂ (58.5 mg, 0.08 mmol, 0.1 eq.) and KOAc (220.8 mg, 2.25 mmol, 3.0 eq.) were added to a mixture of INT C4-7 (400.0 mg, 0.75 mmol, 1.0 eq.), Bis(pinacolato)diborane (382.8 mg, 1.51 mmol, 2.0 eq.) and toluene (4 mL). The reaction mixture was stirred at 110° C. for 3 hrs under N₂ atmosphere, cooled to room temperature after the reaction was completed, filtered and concentrated under reduced pressure to obtain a residue. The residue was purified with silica gel chromatography (eluting with petroleum ether/EtOAc=30:1) to afford INT C4 (202.0 mg, 53.0%) as a yellow solid. ¹H NMR (300 MHz, CDCl₃): δ 7.59 (d, J=8.4 Hz, 1H), 7.46 (d, J=1.8 Hz, 1H), 7.33-7.27 (m, 2H), 5.26 (s, 2H), 3.50 (s, 3H), 2.62 (s, 3H), 1.58 (s, 12H), 1.18 (s, 21H). LCMS: 509.2 ([M+H]⁺).

### Intermediate C5 (INT C5)

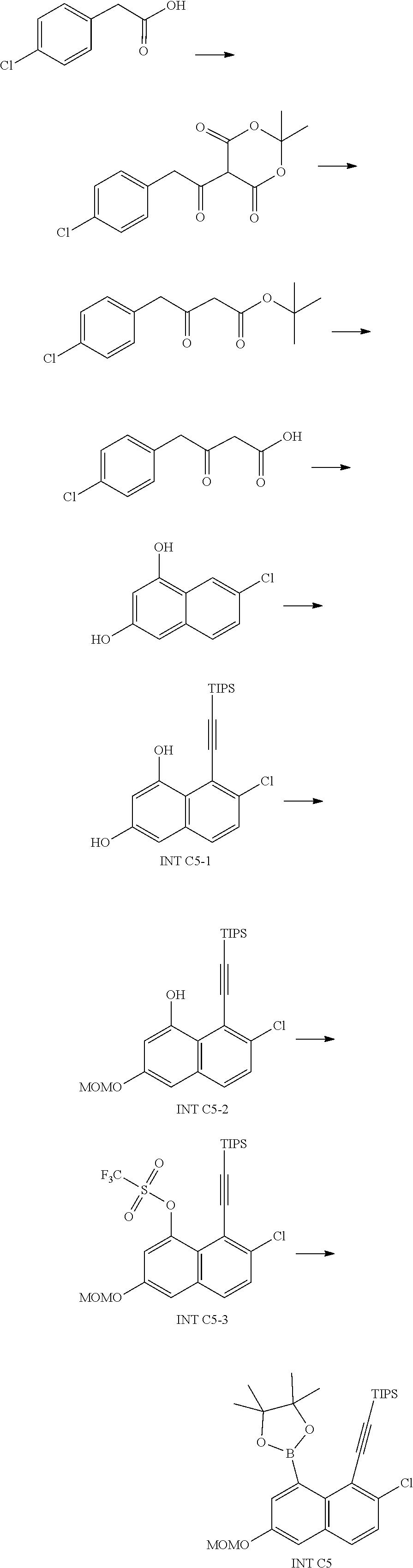

Following an analogous procedure described in procedure of INT C4, 7-chloronaphthalene-1,3-diol was sysnetied with 2-(4-chlorophenyl)acetic acid as start material.

Bis(dichloro(η6-p-cymene)ruthenium) (0.41 g, 0.67 mmol) and potassium acetate (1.63 g, 16.61 mmol) were added to a solution of 7-chloronaphthalene-1,3-diol (1.23 g, 6.32 mmol) and (bromoethynyl)triisopropylsilane (1.94 g, 7.43 mmol) in 1,4-dioxane (20 mL) at R.T. The reaction mixture was purged with nitrogen, stirred at 100° C. for 18 hrs, cooled to R.T, and then filtered. The filtrate was concentrated under reduced pressure to obtain a residue which was purified with silica gel chromatography (EA:Hex=1:25, v/v) to afford INT C5-1 (1.77 g, 74.7%).

Bromomethyl methyl ether (0.79 g, 6.32 mmol) was added dropwise to a solution of INT C5-1 (1.77 g, 4.72 mmol) and DIEA (2.03 g, 15.71 mmol) in DCM (20 mL) at 0° C. The reaction mixture was stirred at 0° C. for 0.5 h, and then quenched with water (50 mL). The organic layer was concentrated under reduced pressure to afford a crude product containing INT C5-2 (1.97 g) which was used to next step directly without further purification.

Tf₂O (2.04 g, 7.23 mmol) was added dropwise to a solution of the crude product containing INT C5-2 (1.97 g) and DIEA (1.91 g, 14.81 mmol) in DCM (80 mL) at −45° C. The reaction mixture was stirred for 3 hrs at −45° C., warmed to R.T and quenched with water (10 mL). The organic layer was concentrated under reduced pressure to obtain a residue which was purified with silica gel chromatography (EA:Hex=1:20-1:10, v/v) to give INT C5-3 (1.94 g, the yield of the above two steps is 74.88%).

A mixture of INT C5-3 (1.98 g, 3.59 mmol), 4,4,4′,4′,5,5,5′,5′-octamethyl-2,2′-bi(1,3,2-dioxaborolane) (1.52 g, 5.99 mmol), Pd(dppf)Cl₂ (0.30 g, 0.41 mmol), potassium acetate (1.30 g, 13.25 mmol) and toluene (20 mL) was purged with nitrogen, stirred at 110° C. for 3.5 hrs, cooled to R.T, and then filtered. The filtrate was concentrated under reduced pressure to obtain a residue which was purified with silica gel chromatography (EA:Hex=1:0-10:1, v/v) to afford INT C5 (232 mg, 12.2%).

### Example 1

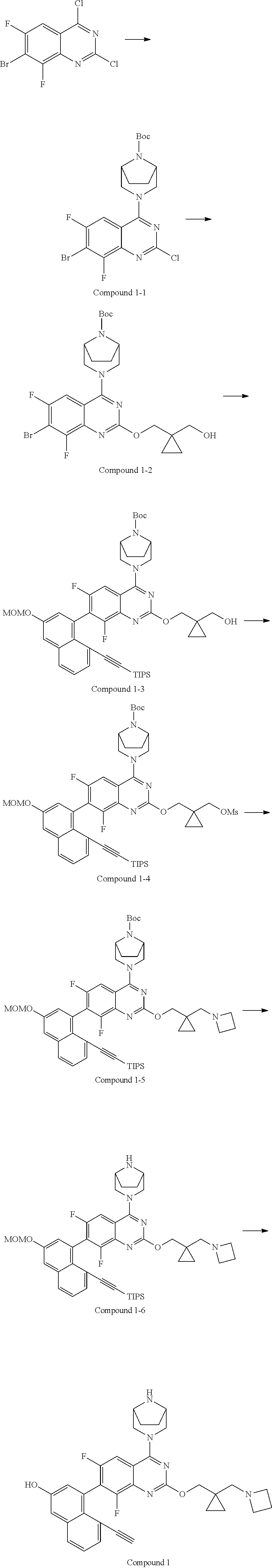

DIEA (8.01 g, 61.98 mmol) was added to a solution of 7-bromo-2,4-dichloro-6,8-difluoroquinazoline (9.39 g, 29.91 mmol) and tert-butyl 3,8-diazabicyclo[3.2.1]octane-8-carboxylate (6.33 g, 29.82 mmol) dissolved in DCM (100 mL) at room temperature. The reaction mixture was stirred for 70 mins, and then water (50 mL) was added. The organic layers were collected, and concentrated under reduced pressure to obtain a residue. The residue was dispersed in EA (15 mL) and Hex (100 mL), and then filtered to afford Compound 1-1 as a white solid (15.13 g, 103.3%).

Compound 1-1 (13.5 g, 27.6 mmol, 1.0 eq.), cyclopropane-1,1-diyldimethanol (8.45 g, 82.6 mmol, 3.0 eq.), triethylenediamine (3.1 g, 27.6 mmol, 1.0 eq.) and Cs₂CO₃ (27 g, 82.6 mmol, 3.0 eq.) were added successively into a mixed solution of THF and DMF (V_{THF}/V_{DMF}=1:1, 200 mL). The reaction mixture was stirred at room temperature for 5 hrs, poured into water (200 mL) after the reaction was completed, and then extracted with EtOAc (200 mL). The organic layer was washed with brine (50 mL), dried with Na₂SO₄, and then concentrated to obtain a residue which was purified with silica gel chromatography (eluting with petroleum ether/EtOAc=5:1) to afford Compound 1-2 (9.75 g, yield 63.6%) as a white solid. LCMS: 555 ([M+H]⁺).

Compound 1-2 (11.0 g, 20.0 mmol, 1.0 eq.), INT C1 (14.6 g, 29.7 mmol, 1.5 eq.), K₃PO₄ (12.6 g, 60.1 mmol, 3.0 eq.) and cataCxium A Pd G₃ (2.16 g, 3.1 mmol, 0.15 eq.) were added successively into a mixed solution of THF and water (V_{THF}/V_{water}=5:1, 120 mL) at R.T. The reaction mixture was purged with argon for 10 mins, stirred at 60° C. for 4 hrs, cooled to room temperature, poured into water (120 mL) and extracted with EtOAc (120 mL×2). The organic layers were combined, washed with brine (60 mL), dried with anhydrous Na₂SO₄, and concentrated to obtain a residue which was purified with silica gel chromatography (eluting with petroleum ether/EtOAc=3:1) to afford Compound 1-3 (5.2 g, yield 30.5%) as a yellow solid. LCMS: 843 ([M+H]⁺).

A mixture of Compound 1-3 (2.0 g, 2.37 mmol, 1.0 eq.), TEA (720.0 mg, 7.11 mmol, 3.0 eq.) and DCM (20 mL) was stirred for 5 mins at R.T, and then methanesulfonyl chloride (543 mg, 4.74 mmol, 2.0 eq.) was added dropwise below 0° C. The reaction mixture was stirred at R.T for 1 h, poured into water (30 mL) and extracted with DCM (30 mL). The organic layer was washed with brine (30 mL), dried with anhydrous Na₂SO₄, and concentrated to obtain a residue which was purified with silica gel chromatography to afford Compound 1-4 (1.9 g, yield 86.9%) as a yellow solid. LCMS: 921 ([M+H]⁺).

A mixture of Compound 1-4 (184.0 mg, 0.20 mmol, 1.0 eq.), azetidine (22.8 mg, 0.40 mmol, 2.0 eq.), TEA (60.6 mg, 0.60 mmol, 3.0 eq.) and DMF (2 mL) was stirred at 80° C. for 2 hrs, cooled to room temperature, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (eluting with ether/EtOAc=3:1) to afford Compound 1-5 (90 mg, yield 51.0%) as a yellow oil. LCMS: 882 ([M+H]⁺).

A solution of Compound 1-5 (80 mg, 0.07 mmol, 1.0 eq.) and HCl/Dioxane (0.6 mL) in MeCN (2 mL) was stirred at R.T for 1 h, and then concentrated under reduced pressure to afford a crude product containing Compound 1-6 (80 mg) as a yellow solid. LCMS: 738 ([M+H]⁺).

A mixture of the crude product containing Compound 1-6 (80 mg), CsF (228 mg, 1.5 mmol, 15.0 eq.) and DMF (2 mL) was stirred overnight at R.T, and then filtered. The filtrate was purified with Prep-HPLC to afford Compound 1 (16.3 mg, yield 26.2%) as a yellow solid.

¹H NMR (400 MHz, CD₃OD-d₄): δ 7.82 (d, J=8 Hz, 1H), 7.55-7.37 (m, 3H), 7.33-7.32 (m, 1H), 7.10-7.03 (m, 1H), 4.43-4.24 (m, 4H), 4.14-4.10 (m, 3H), 3.3-3.56 (m, 4H), 3.31-3.30 (m, 3H), 3.22-2.91 (m, 1H), 2.43-2.41 (m, 2H), 1.90-1.83 (m, 4H), 0.90-0.72 (m, 4H). LCMS: 582.20 ([M+H]⁺).

### Example 2

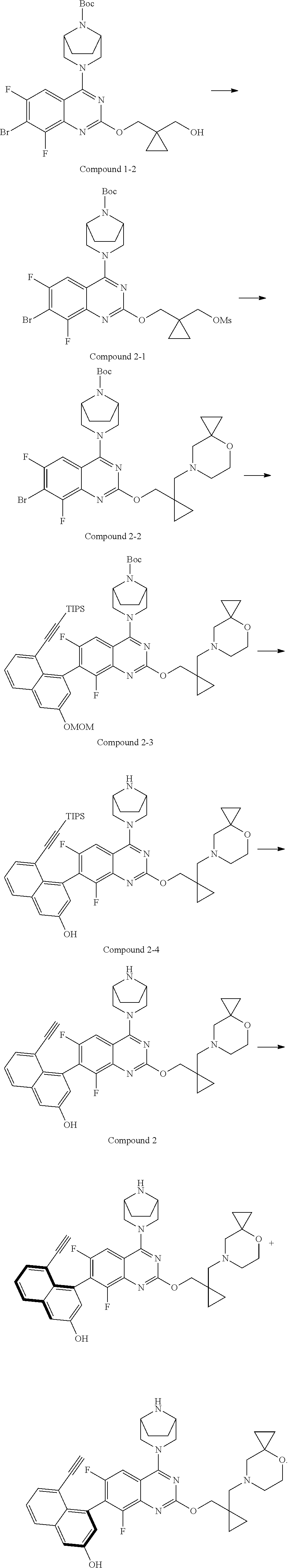

To a mixture of Compound 1-2 (1.20 g, 2.16 mmol) and TEA (0.69 g, 6.81 mmol) in DCM (25 mL) was added MsCl (0.62 g, 5.41 mmol). The reaction mixture was stirred at 0° C. for 0.5 hrs, diluted with DCM (10 mL) and then washed with water (30 mL). The organic layer was dried over anhydrous Na₂SO₄, and concentrated under reduced pressure to afford Compound 2-1 (1.53 g, 2.42 mmol). MS: m/z 633 [M+1]⁺.

A mixture of Compound 2-1 (301 mg, 0.48 mmol), 4-oxa-7-azaspiro[2.5]octane hydrochloride (145 mg, 0.97 mmol), TEA (204 mg, 2.01 mmol) and DMF (5 mL) was stirred at 90° C. for 18 hrs, and then water (30 mL) was added after the reaction was completed. The resulting mixture was extracted with EA. The organic layer was washed with brine, dried over anhydrous Na₂SO₄, concentrated under reduced pressure to afford a residue which was purified with Pre-TLC (Hex:EA=2:1, v/v) to afford Compound 2-2 (169 mg, 0.26 mmol). MS: m/z 650 [M+1]⁺.

A mixture of Compound 2-2 (169 mg, 0.26 mmol), INT C1 (157 mg, 0.32 mmol), Cs₂CO₃ (262 mg, 0.80 mmol), cataCXium A Pd G₃ (21 mg, 0.029 mmol), toluene (8 mL) and water (2 mL) was purged with nitrogen atmosphere, stirred at 100° C. for 15 hrs, and then concentrated under reduced pressure to obtain a residue. The residue was diluted with EA (40 mL) and water (30 mL), and the organic layer was concentrated under reduced pressure to afford a crude product. The crude product was purified with Pre-TLC (Hex:EA=2:1, v/v) to afford Compound 2-3 (204 mg, 0.22 mmol). MS: m/z 938 [M+1]⁺.

A mixture of Compound 2-3 (204 mg, 0.22 mmol), TFA (1.5 mL) and DCM (5 mL) was stirred at 0° C. for 4 hrs, and then concentrated under reduced pressure to obtain a residue. The pH of the mixture of the residue, EA (40 mL) and water (30 mL) was adjusted to 9-10 with solid NaHCO₃, and then the organic layer was concentrated under reduced pressure to afford Compound 2-4 (166 mg, 0.21 mmol). MS: m/z 794 [M+H]⁺.

A mixture of Compound 2-4 (166 mg, 0.21 mmol), CsF (373 mg, 2.45 mmol) and DMF (5 mL) was stirred at R.T for 24 hrs. The pH of the resulting mixture diluting with EA (40 mL) and water (30 mL) was adjusted to 9-10 with aq. NaHCO₃ (5%). The organic layer was concentrated under reduced pressure to obtain a residue which was purified with Pre-HPLC (C18 column; eluent A: 0.1% TFA in water; eluent B: CH₃CN; gradient: 10% B to 40% B in 40 min at a flow rate of 60 mL/min; 240 nm) to afford Compound 2 (12.6 mg, 0.015 mmol). MS: m/z 638 [M+H]⁺.

The enantio separation of the Compound 2 was performed by chiral-HPLC with the following condition: CHIRAL ART Amylose-SA column on Prep-HPLC-Gilson; Mobile phase: Hex (0.1% IPA.M)/EtOH (50:50); Flow rate: 20 mL/min. This resulted in a first eluting stereoisomer (Compound 2A, 3.8 mg, Retention Time 4.266 min) and a second eluting stereoisomer (Compound 2B, 4.2 mg, Retention Time 6.685 min).

### Example 3

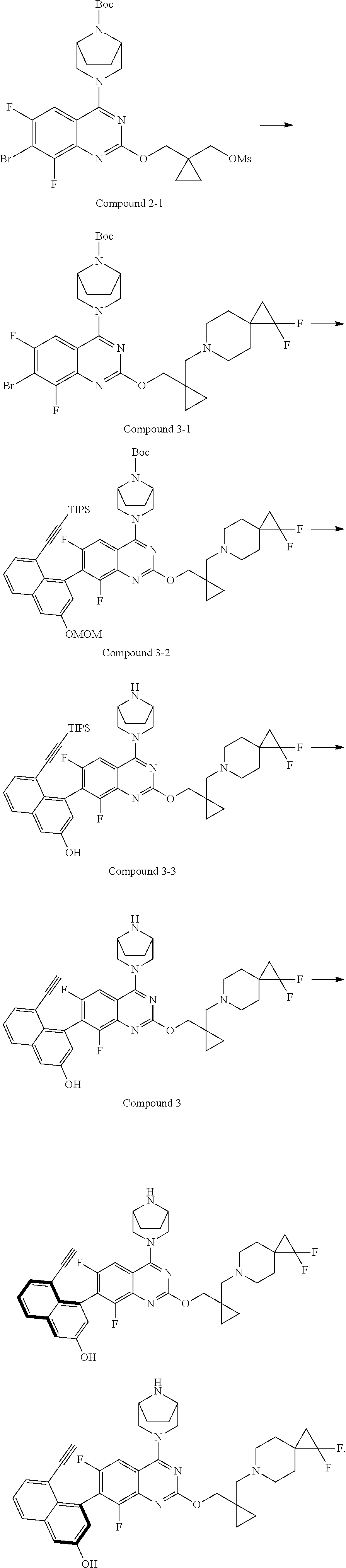

A mixture of 1,1-difluoro-6-azaspiro[2.5]octane hydrochloride (172 mg, 0.94 mmol), TEA (190 mg, 1.88 mmol) and DMF (5 mL) was stirred at RT for 1 h and then Compound 2-1 (306 mg, 0.48 mmol) was added. The reaction mixture was stirred at 90° C. for 18 hrs, diluted with water (30 mL), and then extracted with EA. The organic layer was washed with brine, dried over anhydrous Na₂SO₄, and concentrated under reduced pressure to obtain a residue which was purified with Pre-TLC (Hex:EA=2:1) to afford Compound 3-1 (129 mg, 0.29 mmol). MS: m/z 684 [M+1]⁺.

A mixture of Compound 3-1 (129 mg, 0.29 mmol), INT C1 (115 mg, 0.23 mmol), Cs₂CO₃ (191 mg, 0.59 mmol), cataCXium A Pd G₃ (15 mg, 0.021 mmol), toluene (8 mL) and water (2 mL) was purged with nitrogen, stirred at 100° C. for 16 hrs, and then concentrated under reduced pressure to obtain a residue. The residue was diluted with EA (40 mL) and water (30 mL), and the organic layer was concentrated under reduced pressure to obtain a crude product which was purified with Pre-TLC (Hex:EA=2:1, v/v) to afford Compound 3-2 (161 mg, 0.17 mmol). MS: m/z 972 [M+1]⁺.

A solution of Compound 3-2 (155 mg, 0.16 mmol) and HCl/1, 4-dioxane (4 M, 2 mL) in CH₃CN (5 mL) was stirred at R.T for 1 h, and then concentrated under reduced pressure to obtain a residue. The pH of the mixture of the residue, EA (40 mL) and water (30 mL) was adjusted to 8-9 with aq. NaHCO₃ (5%). The organic layer was washed with brine, dried over anhydrous Na₂SO₄, and concentrated under reduced pressure to afford Compound 3-3 (137 mg, 0.17 mmol). MS: m/z 828 [M+H]⁺.

A mixture of Compound 3-3 (137 mg, 0.17 mmol), CsF (410 mg, 2.70 mmol) and DMF (5 mL) was stirred at R.T for 17 hrs, and then filtered. The filtrate was concentrated under reduced pressure to obtain a residue which was purified with Pre-HPLC (C18 column; eluent A: 0.10% TFA in water; eluent B: CH₃CN; gradient: 15% B to 45% B in 35 min at a flow rate of 60 mL/min; 240 nm) to afford Compound 3 (95.9 mg, 0.11 mmol). MS: m/z 672 [M+H]⁺.

The enantio separation of the Compound 3 was performed by chiral-HPLC with the following condition: CHIRAL ART Amylose-SA column on Prep-HPLC-Gilson; Mobile phase: Hex (0.1% IPA.M)/EtOH (50:50); Flow rate: 20 mL/min. This resulted in a first eluting stereoisomer (Compound 3A, 17.3 mg, Retention Time 3.665 min) and a second eluting stereoisomer (Compound 3B, 25.4 mg, Retention Time 5.532 min).

### Example 4

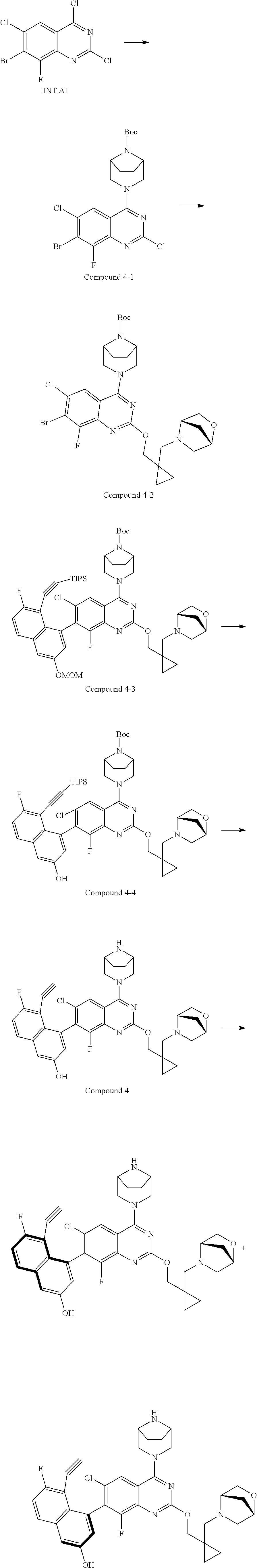

A solution of INT A1 (300 mg, 0.9 mmol), Et₃N (273 mg, 2.7 mmol) and tert-butyl (1R,5S)-3,8-diazabicyclo[3.2.1]octane-8-carboxylate (191 mg, 0.9 mmol) in 1,4-dioxane (3 mL) was stirred at room temperature for 0.5 h, and then concentrated under vacuum to obtain a residue. The residue was dissolved in DCM (200 mL), washed with brine (2×100 mL), dried over Na₂SO₄ and concentrated in vacuum to obtain a crude product which was purified with Prep-TLC (EA/Hex=1:5, v/v) to afford the desired product Compound 4-1 (320 mg) as an off-white solid. MS m/z: 505 [M+H]⁺.

A solution of Compound 4-1 (302 mg, 596.60 μmol), INT B1 (133 mg, 725.79 Mol), DABCO (35 mg, 312.02 μmol) and Cs₂CO₃ (573 mg, 1.75 mmol) in THF (3 mL) and DMF (3 mL) was stirred at room temperature for 16 hrs, concentrated under vacuum, diluted with water (50 mL), and then extracted with EA (2×50 mL). aq. HCl (50 mL, 1 M) was added to the combined organic layers; The pH of the water layer was adjusted to 8 with NaHCO₃, and the water layer was extracted with EA (2×50 mL). The organic layers were combined, dried over Na₂SO₄ and concentrated under vacuum to afford Compound 4-2 (217 mg, 332.32 μmol, 55.7% yield). MS (ESI, m/z): 652 [M+H]⁺.

A mixture of Compound 4-2 (373 mg, 571.22 μmol), INT C2 (450 mg, 841.98 μmol), cataCXium A Pd G₃, (53 mg, 72.77 μmol), Cs₂CO₃ (592 mg, 1.81 mmol) in toluene (8 mL) and water (2 mL) was stirred at 100° C. for 16 hrs under an atmosphere of nitrogen, diluted with EA (50 mL) and then washed with water (3×30 mL). The organic layer was dried over Na₂SO₄ and concentrated under vacuum to obtain a residue which was purified with Pre-TLC (EA:Hex=2:1, v/v) to afford Compound 4-3 (302 mg, 315.02 μmol, 55.2% yield). MS (ESI, m/z): 958 [M+H]⁺.

To a solution of Compound 4-3 (302 mg, 315.02 μmol) in CH₃CN (6 mL) was added HCl (4 M in 1,4-dioxane, 1.5 mL). The reaction mixture was stirred at room temperature for 1 h. The resulting mixture was concentrated under reduced pressure to afford Compound 4-4 (262 mg, 321.67 μmol, 102.1% yield). MS m/z: 814 [M+H]⁺.

To a solution of Compound 4-4 (262 mg, 321.67 μmol) in DMF (3 mL) was added CsF (0.63 g, 4.14 mmol) and the reaction mixture was stirred at 40° C. for 16 hrs. The resulting mixture was concentrated under reduced pressure and the residue was purified with Pre-HPLC (Daisogel-C18 column, 100*250 mm, 10 μm, A: 0.1% TFA in water, B: CH₃CN, gradient: 15% B to 60% B in 35 min at a flow rate of 200 mL/min, 240 nm) to afford Compound 4 (153 mg, 232.47 μmol, 72.2% yield). MS m/z: 658 [M+H]⁺.

Compound 4 (153 mg, 232.47 μmol) was separated with Prep-HPLC-Gilson with the following conditions: Column, CHIRAL ART Cellulose-SA column (2 cm×25 cm, 5 um); mobile phase, Hex (0.1% IPA.M)/EtOH (50:50); flowing rate: 20 ml/min to afford Compound 4A (44.2 mg, Ret Time 4.561 min) and Compound 4B (55.8 mg, Ret Time 7.002 min) respectively.

### Example 5

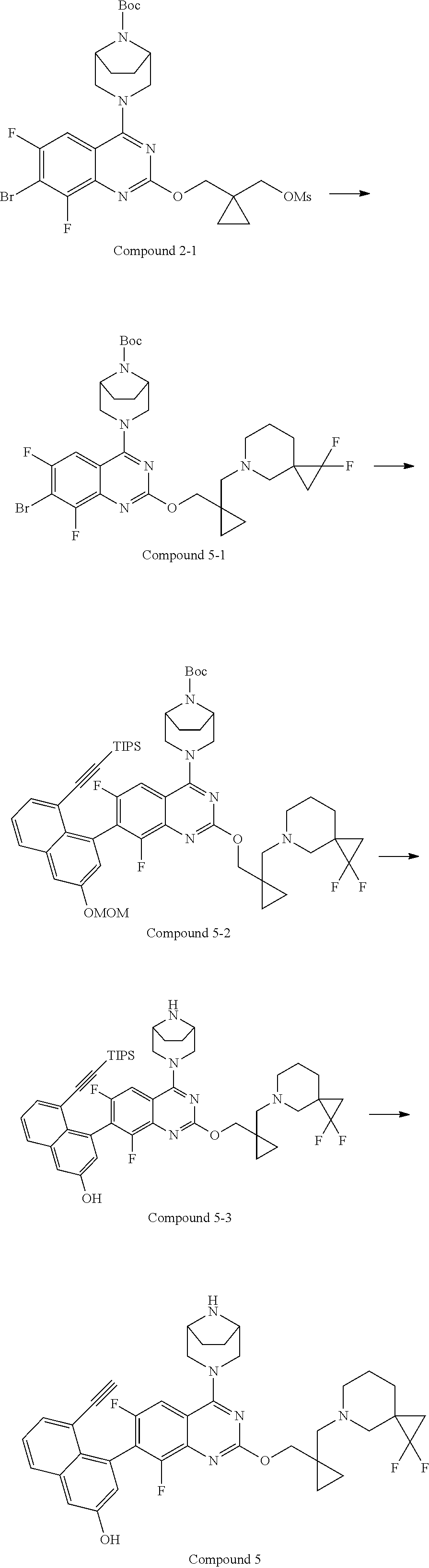

A mixture of 1,1-difluoro-5-azaspiro[2.5]octane hydrochloride (170 mg, 0.93 mmol), TEA (190 mg, 1.88 mmol) and DMF (5 mL) was stirred at R.T for 1 h, and then Compound 2-1 (309 mg, 0.49 mmol) was added. The mixture was stirred at 90° C. for 18 hrs, and then water (30 mL) was added. The resulting mixture was extracted with EA. The organic layers were combined, washed with brine, dried over anhydrous Na₂SO₄, and concentrated under reduced pressure to obtain a residue which was purified with Pre-TLC (Hex:EA=2:1, v/v) to afford Compound 5-1 (188 mg, 0.27 mmol). MS: m/z 684 [M+1]⁺.

A mixture of Compound 5-1 (233 mg, 0.34 mmol), INT C1 (192 mg, 0.39 mmol), cataCXium A Pd G₃ (28 mg, 0.038 mmol), Cs₂CO₃ (331 mg, 1.02 mmol), toluene (8 mL) and water (2 mL) was purged with nitrogen, stirred at 100° C. for 17 hrs, and then concentrated under reduced pressure to obtain a residue. The residue was diluted with EA (40 mL) and water (30 mL), and the organic layer was concentrated under reduced pressure to obtain a residue which was purified with Pre-TLC (Hex:EA=2:1, v/v) to afford Compound 5-2 (259 mg, 0.27 mmol). MS: m/z 972.45 [M+1]⁺.

A solution of Compound 5-2 (259 mg, 0.27 mmol), HCl/1, 4-dioxane (4 M, 2 mL) and CH₃CN (5 mL) was stirred at R.T for 1 h, and then concentrated under reduced pressure to obtain a residue. The pH of the mixture of the residue, EA (40 mL) and water (30 mL) was adjusted to 8-9 with aq. NaHCO₃ (5%). The organic layer was washed with brine, dried over anhydrous Na₂SO₄, and concentrated under reduced pressure to afford Compound 5-3 (222 mg, 0.27 mmol). MS: m/z 828 [M+H]⁺.

A mixture of Compound 5-3 (222 mg, 0.27 mmol), CsF (279 mg, 1.84 mmol) and DMF (5 mL) was stirred at R.T for 18 hrs, and then filtered. The filtrate was concentrated under reduced pressure to obtain a residue which was purified with Pre-HPLC (C18 column; eluent A: 0.1% TFA in water; eluent B: CH₃CN; gradient: 15% B to 60% B in 40 min at a flow rate of 60 mL/min; 230 nm) to afford Compound 5 (164.1 mg, 0.18 mmol). MS: m/z 672 [M+H]⁺.

### Example 6

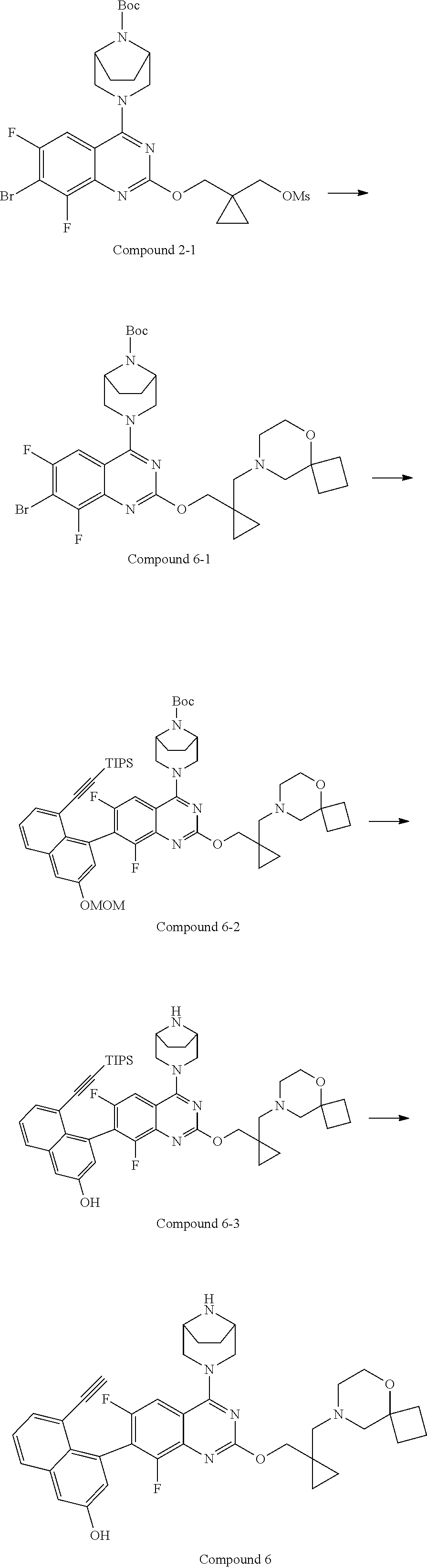

A mixture of Compound 2-1 (252 mg, 0.40 mmol), 5-oxa-8-azaspiro[3.5]nonane (80 mg, 0.63 mmol), TEA (123 mg, 1.22 mmol) and DMF (5 mL) was stirred at 90° C. for 17 hrs, and then water (30 mL) was added. The reaction mixture was extracted with EA (40 mL). The organic layer was washed with brine and concentrated under reduced pressure to obtain a residue which was purified with Pre-TLC (Hex:EA=2:1, v/v) to afford Compound 6-1 (82 mg, 0.12 mmol). MS: m/z 664 [M+1]⁺.

A mixture of Compound 6-1 (82 mg, 0.12 mmol), INT C1 (76 mg, 0.15 mmol), cataCXium A Pd G₃ (10 mg, 0.014 mmol), Cs₂CO₃ (126 mg, 0.39 mmol), toluene (4 mL) and water (1 mL) was purged with nitrogen, stirred at 100° C. for 17 hrs, and then concentrated under reduced pressure to obtain a residue. The residue was diluted with EA (40 mL) and water (30 mL), and the organic layer was concentrated under reduced pressure to obtain a crude product which was purified with Pre-TLC (Hex:EA=2:1, v/v) to afford Compound 6-2 (105 mg, 0.11 mmol). MS: m/z 952 [M+1]⁺.

A solution of Compound 6-2 (105 mg, 0.11 mmol), HCl/1, 4-dioxane (4 M, 2 mL) in CH₃CN (5 mL) was stirred at R.T for 1 h, and then concentrated under reduced pressure to obtain a residue. The residue was diluted with EA (40 mL) and water (30 mL) and the pH of the resulting mixture was adjusted to 8-9 with aq. NaHCO₃ (5%). The organic layer was washed with brine, dried over anhydrous Na₂SO₄, and concentrated under reduced pressure to afford Compound 6-3 (87 mg, 0.11 mmol). MS: m/z 808 [M+H]⁺.

A mixture of Compound 6-3 (87 mg, 0.11 mmol), CsF (336 mg, 2.21 mmol) and DMF (5 mL) was stirred at R.T for 18 hrs, and then diluted with EA (40 mL) and water (30 mL). The pH of the resulting mixture was adjusted to 8-9 with aq. NaHCO₃ (5%). The organic layer was concentrated under reduced pressure which was purified with Pre-HPLC (C18 column; eluent A: 0.1% TFA in water; eluent B: CH₃CN; gradient: 15% B to 45% B in 40 min at a flow rate of 60 mL/min; 240 nm) to afford Compound 6 (66.6 mg, 0.076 mmol). MS: m/z 652 [M+H]⁺.

### Example 7

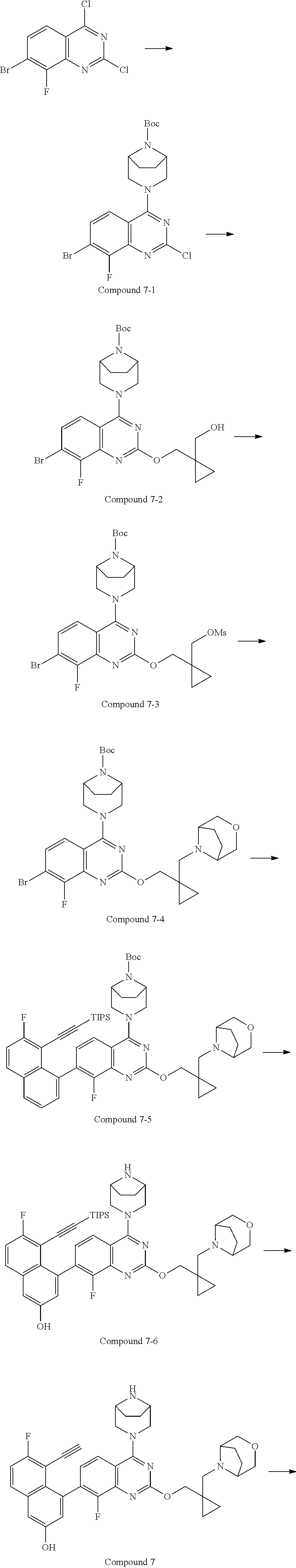

To a solution of INT A3 (7.08 g, 23.93 mmol) and tert-butyl (1R,5S)-3,8-diazabicyclo[3.2.1]octane-3-carboxylate (4.93 g, 23.22 mmol) in DCM (100 mL) was added DIEA (5.98 g, 46.27 mmol) at room temperature. The reaction mixture was stirred for 50 min, then water (50 mL) was added. The organic phase was collected, and concentrated under reduced pressure. The obtained residue was suspended in EA (15 mL) and Hex (100 mL), then filtered. The filter cake was collected and dried to give Compound 7-1 as a white solid (10.56 g, 93.6%). LCMS [M+1]=471.

Compound 7-1 (6.0 g, 12.7 mmol, 1.0 eq.), cyclopropane-1,1-diyldimethanol (3.9 g, 38.1 mmol, 3.0 eq.), triethylenediamine (1.42 g, 12.7 mmol, 1.0 eq.) and Cs₂CO₃ (12.4 g, 38.1 mmol, 3.0 eq.) were added successively into a mixed solution of THF and DMF (V_{THF}/V_{DMF}=1:1, 120 mL). The reaction mixture was stirred overnight at R.T, poured into water (60 mL) and extracted with EtOAc (60 mL). The organic layer was washed with brine (30 mL), dried with anhydrous Na₂SO₄, and concentrated to obtain a residue. The residue was slurried with EtOAc and then filtered to afford Compound 7-2 (5.2 g, yield 76.1%) as a white solid. LCMS: 537 ([M+H]⁺).

To a mixture of Compound 7-2 (1.93 g, 3.59 mmol), TEA (1.13 g, 11.17 mmol) and DCM (50 mL) was added MsCl (0.92 g, 8.03 mmol). The reaction mixture was stirred at 0° C. for 1 h, diluted with DCM (50 mL) and washed with water (60 mL). The organic layer was dried over anhydrous Na₂SO₄, and concentrated under reduced pressure to afford Compound 7-3 (2.19 g, 2.21 mmol). MS: m/z 615 [M+1]⁺.

A mixture of 3-oxa-8-azabicyclo[3.2.1]octane hydrochloride (172 mg, 1.15 mmol), TEA (254 mg, 2.51 mmol) and DMF (5 mL) was stirred at R.T for 1 h, and then Compound 7-3 (354 mg, 0.58 mmol) was added. The reaction mixture was stirred at 90° C. for 16 hrs, and then extracted with EA (40 mL) after water (30 mL) was added. The organic layer was washed with brine, dried over anhydrous Na₂SO₄, and concentrated under reduced pressure to obtain a residue which was purified with Pre-TLC (Hex:EA=1:1, v/v) to afford Compound 7-4 (163 mg, 0.26 mmol). MS: m/z 632 [M+1]⁺.

A mixture of Compound 7-4 (163 mg, 0.26 mmol), INT C2 (182 mg, 0.36 mmol), Cs₂CO₃ (252 mg, 0.77 mmol), Pd(dppf)Cl₂ (22 mg, 0.030 mmol), 1, 4-dioxane (8 mL) and water (2 mL) was purged with nitrogen, stirred at 100° C. for 3 hrs, and then concentrated under reduced pressure to obtain a residue. The residue was diluted with EA (30 mL) and water (30 mL), and the organic layer was concentrated under reduced pressure to obtain a crude product which was purified with Pre-TLC (Hex:EA=2:3, v/v) to afford Compound 7-5 (87 mg, 0.093 mmol). MS: m/z 938 [M+H]⁺.

A solution of Compound 7-5 (87 mg, 0.093 mmol) and HCl/1, 4-dioxane (4 M, 1.5 mL) in CH₃CN (5 mL) was stirred at R.T for 2 hrs, and then concentrated under reduced pressure to obtain a crude product containing Compound 7-6. MS: m/z 794 [M+H]⁺.

A mixture of the crude product containing Compound 7-6, CsF (272 mg, 1.79 mmol) and DMF (5 mL) was stirred at R.T for 16 hrs, and then filtered. The filtrate was concentrated under reduced pressure to obtain a residue which was purified with Pre-HPLC (C18 column; eluent A: 0.1% TFA in water; eluent B: CH₃CN; gradient: 15% B to 40% B in 35 min at a flow rate of 60 mL/min; 240 nm) to afford Compound 7 (33.0 mg, 0.052 mmol). MS: m/z 638 [M+H]⁺.

### Example 8

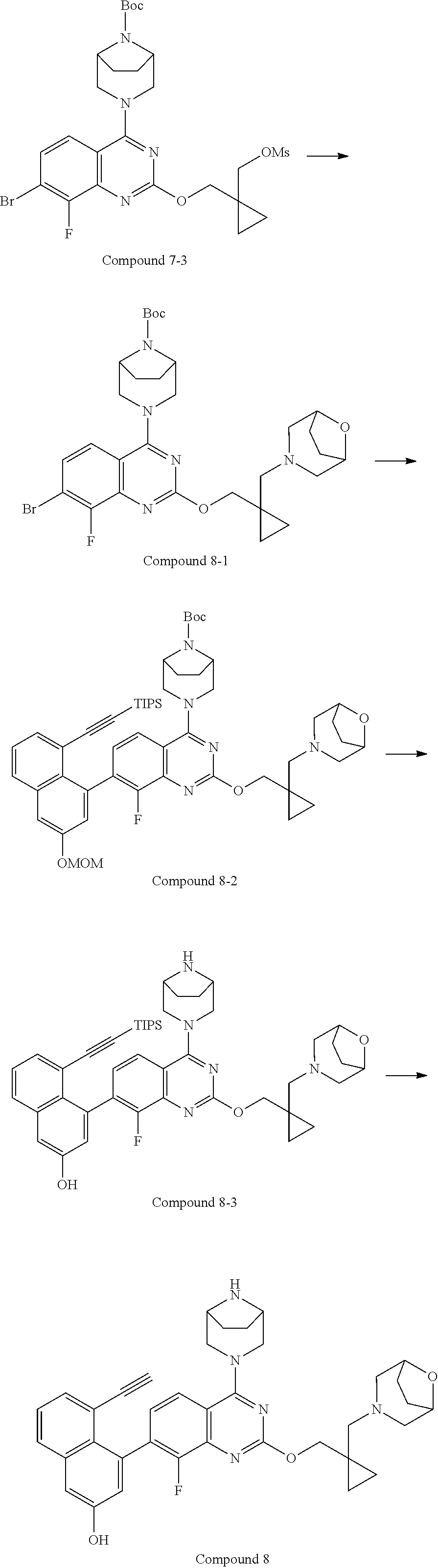

TEA (238 mg, 2.35 mmol) was added to a solution of 8-oxa-3-azabicyclo[3.2.1]octane hydrochloride (172 mg, 1.15 mmol) in DMF (5 mL). The reaction mixture was stirred at R.T for 2 hrs, and then Compound 7-3 (306.1 mg, 0.50 mmol) was added. The resulting mixture was stirred at 90° C. for 16 hrs, diluted with water (30 mL) and then extracted with EA (40 mL). The organic layer was washed with brine, dried over anhydrous Na₂SO₄, and concentrated under reduced pressure to obtain a residue which was purified with Pre-TLC (Hex:EA=2:1, v/v) to afford Compound 8-1 (114 mg, 0.18 mmol). MS: m/z 632 [M+1]⁺.

CataCXium A Pd G₃ (14 mg, 0.019 mmol) was added to a mixture of Compound 8-1 (114 mg, 0.18 mmol), INT C1 (116 mg, 0.23 mmol), K₃PO₄ (162 mg, 0.76 mmol), THF (8 mL) and water (2 mL). The reaction mixture was purged with nitrogen, stirred at 60° C. for 3 hrs, and then concentrated under reduced pressure to obtain a residue. The residue was diluted with EA (40 mL) and water (30 mL), and the organic layer was concentrated under reduced pressure to obtain a crude product which was purified with silica gel chromatography (eluting with Hex:EA=4:1, v/v) to afford Compound 8-2 (136 mg, 0.15 mmol). MS: m/z 920 [M+1]⁺.

A solution of Compound 8-2 (136 mg, 0.15 mmol) and HCl/1, 4-dioxane (4 M, 2 mL) in CH₃CN (5 mL) was stirred at R.T for 1 h, and then concentrated under reduced pressure to obtain a residue. The residue was diluted with EA (40 mL) and water (30 mL) and the pH of the resulting mixture was adjusted to 8-9 with aq. NaHCO₃ (5%). The organic layer was washed with brine, dried over anhydrous Na₂SO₄, and concentrated under reduced pressure to afford Compound 8-3 (119 mg, 0.15 mmol). MS: m/z 776 [M+H]⁺.

A mixture of Compound 8-3 (119 mg, 0.15 mmol), CsF (233 mg, 1.53 mmol) and DMF (5 mL) was stirred at R.T for 20 hrs, and then diluted with EA (40 mL) and water (30 mL). The pH of the resulting mixture was adjusted to 8-9 with aq. NaHCO₃ (5%). The organic layer was separated and concentrated under reduced pressure to obtain a residue which was purified with Pre-HPLC (C18 column; eluent A: 0.1% TFA in water; eluent B: CH₃CN; gradient: 10% B to 40% B in 40 min at a flow rate of 60 mL/min; 234 nm) to afford Compound 8 (82.7 mg, 0.098 mmol). MS: m/z 620 [M+H]⁺.

### Example 9

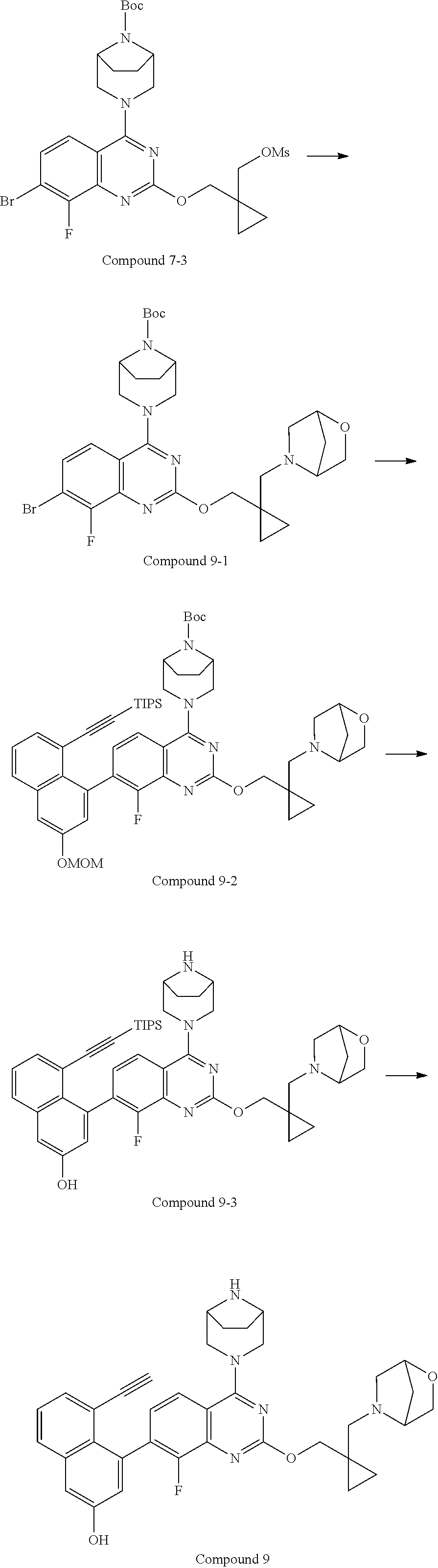

TEA (212 mg, 2.10 mmol) was added to a solution of 2-oxa-5-azabicyclo[2.2.1]heptane hydrochloride (132 mg, 1.33 mmol) in DMF (5 mL), and then the mixture was stirred at R.T for 1.5 hrs. After adding Compound 7-3 (305 mg, 0.50 mmol), the reaction mixture was stirred at 90° C. for 24 hrs, diluted with water (30 mL), and extracted with EA (40 mL). The organic layer was washed with brine, dried over anhydrous Na₂SO₄, and concentrated under reduced pressure to obtain a residue which was purified with Pre-TLC (EA) to afford Compound 9-1 (162 mg, 0.26 mmol). MS: m/z 618 [M+1]⁺.

CataCXium A Pd G₃ (20 mg, 0.027 mmol) was added to a solution of Compound 9-1 (162 mg, 0.26 mmol), INT C1 (166 mg, 0.34 mmol), K₃PO₄ (196 mg, 0.92 mmol), THF (8 mL) and water (2 mL). The reaction mixture was purged with nitrogen, stirred at 60° C. for 2 hrs, and concentrated under reduced pressure to obtain a residue. The residue was diluted with EA (35 mL) and water (30 mL), and the organic layer was separated and concentrated under reduced pressure to obtain a crude product which was purified with silica gel chromatography (eluting with Hex:EA=3:1-0:1, v/v) to afford Compound 9-2 (199 mg, 0.22 mmol). MS: m/z 906 [M+1]⁺.

A solution of Compound 9-2 (199 mg, 0.22 mmol) and HCl/1,4-dioxane (4 M, 2 mL) in CH₃CN (5 mL) was stirred at R.T for 2 h, and then concentrated under reduced pressure to obtain a residue. The residue was diluted with EA (40 mL) and water (30 mL), and the pH of the resulting mixture was adjusted to 8-9 with aq. NaHCO₃ (5%). The organic layer was separated, washed with brine, dried over anhydrous Na₂SO₄, and concentrated under reduced pressure to afford Compound 9-3 (166 mg, 0.22 mmol). MS: m/z 762 [M+H]⁺.

A mixture of Compound 9-3 (166 mg, 0.22 mmol), CsF (286 mg, 1.88 mmol) and DMF (5 mL) was stirred at R.T for 16 hrs, and then diluted with EA (40 mL) and water (30 mL). The pH of the resulting mixture was adjusted to 8-9 with aq. NaHCO₃ (5%). The organic layer was separated and concentrated under reduced pressure to obtain a residue which was purified with Pre-HPLC (C18 column; eluent A: 0.1% TFA in water; eluent B: CH₃CN; gradient: 15% B to 35% B in 35 min at a flow rate of 60 mL/min; 240 nm) to afford Compound 9 (95.1 mg, 0.11 mmol). MS: m/z 606 [M+H]⁺.

### Example 10

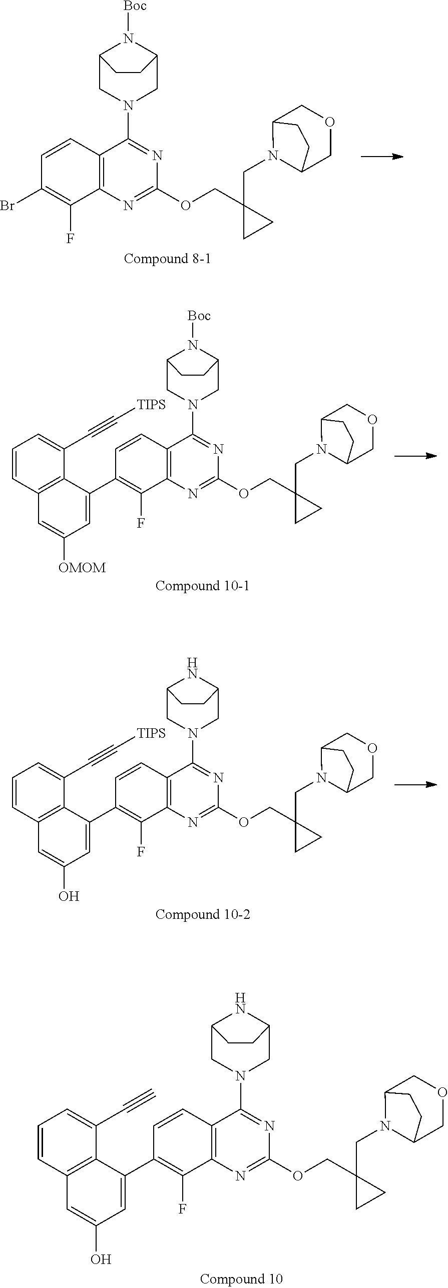

CataCXium A Pd G₃ (13 mg, 0.017 mmol) was added to a mixture of Compound 7-4 (111 mg, 0.18 mmol), INT C1 (101 mg, 0.20 mmol), K₃PO₄ (140 mg, 0.66 mmol), THF (8 mL) and water (2 mL). The reaction mixture was purged with nitrogen, stirred at 65° C. for 18 hrs, and then concentrated under reduced pressure to obtain a residue which was diluted with EA (40 mL) and water (30 mL). The organic layer was separated, and concentrated under reduced pressure to obtain a crude product which was purified with silica gel chromatography (eluting with Hex:EA=4:1˜1:1, v/v) to afford Compound 10-1 (127 mg, 0.14 mmol). MS: m/z 920 [M+H]⁺.

A solution of Compound 10-1 (127 mg, 0.14 mmol), HCl/1,4-dioxane (4 M, 2 mL) in CH₃CN (5 mL) was stirred at R.T for 1 h, and then concentrated under reduced pressure to obtain a residue which was diluted with EA (40 mL) and water (30 mL). The pH of the resulting mixture was adjusted to 9-10 with NaHCO₃. The organic layer was separated and concentrated under reduced pressure to afford Compound 10-2 (96 mg, 0.21 mmol). MS: m/z 776 [M+H]⁺.

A mixture of Compound 10-2 (96 mg, 0.21 mmol), CsF (170 mg, 1.12 mmol) and DMF (5 mL) was stirred at R.T for 18 hrs, and then diluted with EA (40 mL) and water (30 mL). The pH of the resulting mixture was adjusted to 9-10 with aq. NaHCO₃ (5%). The organic layer was separated and concentrated under reduced pressure to obtain a residue which was purified with Pre-HPLC (C18 column; eluent A: 0.1% TFA in water; eluent B: CH₃CN; gradient: 15% B to 40% B in 35 min at a flow rate of 60 mL/min; 290 nm) to afford Compound 10 (16.1 mg, 0.026 mmol). MS: m/z 620 [M+H]⁺.

### Example 11

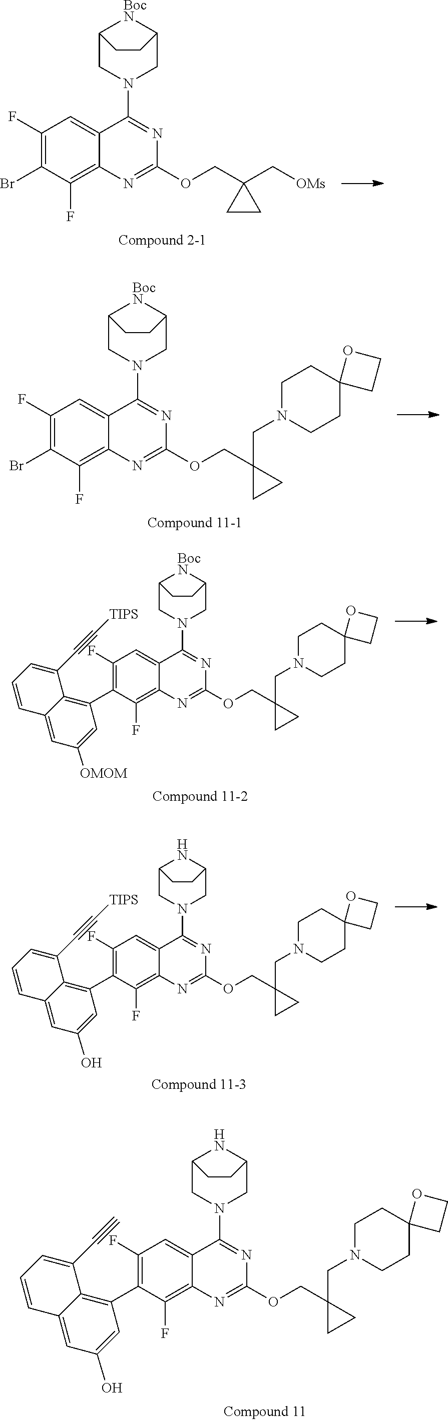

A solution of 1-oxa-7-azaspiro[3.5]nonane hemioxalate (167 mg, 1.31 mmol), triethylamine (0.5 mL) dissolved DMF (5 mL) was stirred at R.T for 2 hrs, then Compound 2-1 (298 mg, 470.40 μmol) was added. The reaction mixture was purged with nitrogen, stirred at 90° C. for 16 hrs, cooled to R.T, diluted with EA (50 mL) and washed with water (2×30 mL). The organic layer was dried over Na₂SO₄ and concentrated under reduced pressure to obtain a residue which was purified with Pre-TLC (EA:Hex=1:2, v/v) to afford Compound 11-1 (100 mg, 150.47 μmol, 31.9% yield). MS m/z: 664 [M+H]⁺.

A mixture of Compound 11-1 (100 mg, 150.47 μmol), INT C1 (115 mg, 232.53 μmol), cataCXium A Pd G₃ (20 mg, 27.46 μmol), Cs₂CO₃ (148 mg, 454.24 μmol), toluene (8 mL) and water (2.0 mL) was stirred at 100° C. for 6 hrs under an atmosphere of nitrogen, diluted with EA (100 mL) and washed with water (2×40 mL). The organic layer was dried over Na₂SO₄ and concentrated under reduced pressure to obtain a residue which was purified with Pre-TLC (MeOH:DCM=1:15, v/v) to afford Compound 11-2 (76 mg, 79.81 μmol, 53.0% yield). MS m/z: 952 [M+H]⁺.

4-toluenesulfonic acid (56 mg, 325.20 μmol) was added to a solution of Compound 11-2 (76 mg, 79.81 μmol) in 1,4-dioxane (3 mL). The reaction mixture was purged with nitrogen, stirred at 60° C. for 16 hrs, diluted with EA (50 mL), washed with saturated aq. NaHCO₃ (2×30 mL). The organic layer was dried over Na₂SO₄ and concentrated under reduced pressure to afford Compound 11-3 (59 mg, 73.01 μmol). MS m/z: 808 [M+H]⁺.

A mixture of Compound 11-3 (59 mg, 73.01 μmol), CsF (172 mg, 1.13 mmol) and DMF (5 mL) was stirred at 30° C. for 16 hrs. The resulting mixture was concentrated under reduced pressure to obtain a residue which was purified with Pre-HPLC (Daisogel-C18 column, 50*250 mm, 10 μm; eluent A: 10 mM/L NH₄HCO₃ in water; eluent B: CH₃CN; gradient: 20% B to 70% B in 50 min at a flow rate of 60 mL/min; 240 nm) to afford Compound 11 (6.6 mg, 10.12 μmol, 13.8% yield). MS m/z: 652 [M+H]⁺.

### Example 12

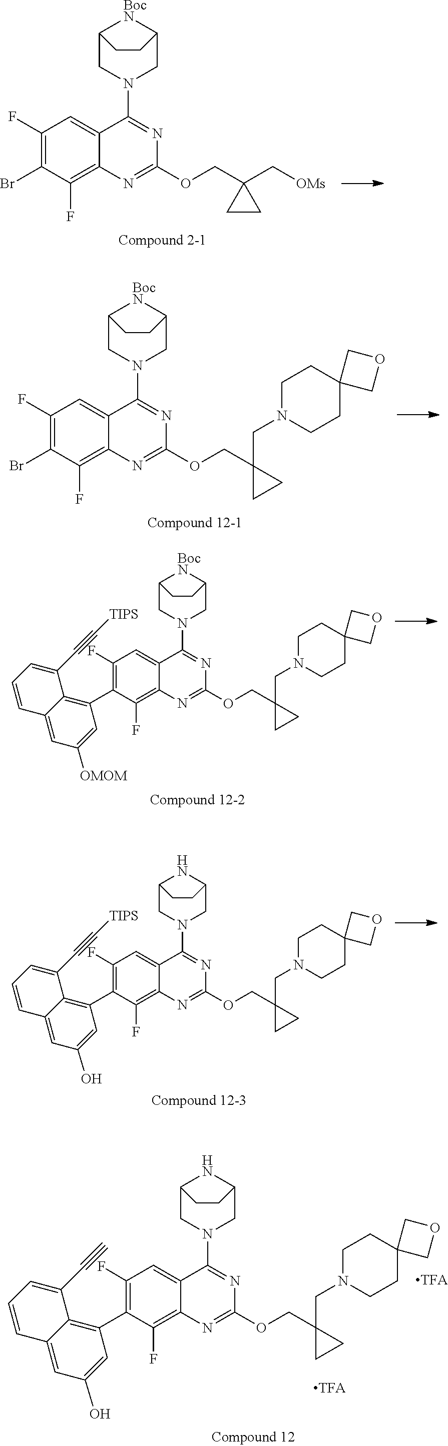

A solution of 2-oxa-7-azaspiro[3.5]nonane oxalate (168 mg, 487.80 μmol) and triethylamine (0.5 mL) in DMF (5 mL) was stirred at room temperature for 1 h, then Compound 2-1 (311 mg, 490.92 μmol) was added. The reaction mixture was purged with nitrogen, stirred at 90° C. for 16 hrs, cooled to R.T, diluted with EA (50 mL) and washed with water (2×30 mL). The organic layer was dried over Na₂SO₄ and concentrated under reduced pressure to obtain a residue which was purified with Pre-TLC (EA:Hex=1:2, v/v) to afford Compound 12-1 (76 mg, 114.35 μmol, 23.2% yield). MS m/z: 664 [M+H]⁺.

A mixture of Compound 12-1 (76 mg, 114.35 μmol), INT C1 (77 mg, 155.69 μmol), cataCXium A Pd G₃ (16 mg, 21.96 μmol), Cs₂CO₃ (118 mg, 362.16 μmol), toluene (8 mL) and water (2 mL) was stirred at 100° C. for 16 hrs under nitrogen atmosphere, diluted with EA (50 mL) and washed with water (2×30 mL). The organic layer was dried over Na₂SO₄ and concentrated under reduced pressure to obtain a residue which was purified with Pre-TLC (MeOH:DCM=1:15, v/v) to afford Compound 12-2 (80 mg, 84.01 μmol, 73.4% yield). MS m/z: 952 [M+H]⁺.

TFA (3 mL) was added to a solution of Compound 12-2 (80 mg, 84.01 μmol) in DCM (5 mL). The reaction mixture was stirred at R.T for 1 h, diluted with DCM (50 mL), washed with saturated aq. NaHCO₃ (2×30 mL). The organic layer was dried over Na₂SO₄ and concentrated under reduced pressure to afford Compound 12-3 (60 mg, 74.24 μmol, 88.3% yield). MS m/z: 808 [M+H]⁺.

CsF (307 mg, 2.02 mmol) was added to a solution of Compound 12-3 (60 mg, 74.24 μmol) in DMF (5 mL). The reaction mixture was stirred at 30° C. for 16 hrs, and then concentrated under reduced pressure to obtain a residue which was purified with Pre-HPLC (Daisogel-C18 column, 50*250 mm, 10 μm; eluent A: 0.1% TFA in water; eluent B: CH₃CN; gradient: 15% B to 45% B in 40 min at a flow rate of 60 mL/min; 240 nm) to afford a TFA salt of Compound 12 (20.8 mg, 23.64 μmol, 31.8% yield). MS m/z: 652 [M+H]⁺.

### Example 13

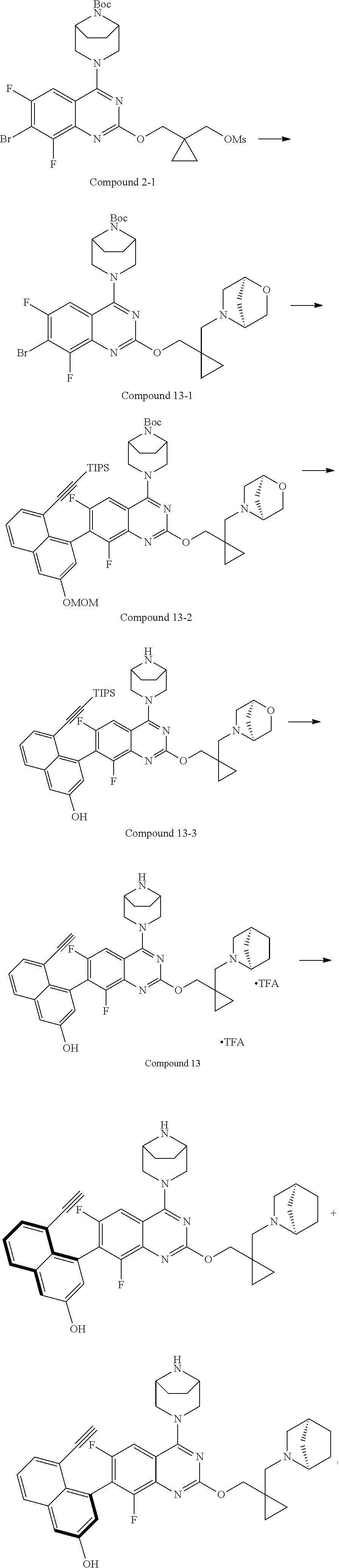

A solution of (1S,4S)-2-oxa-5-azabicyclo[2.2.1]heptane hydrochloride (116 mg, 855.50 μmol), triethylamine (0.3 mL) and Compound 2-1 (268 mg, 423.04 μmol) in DMF (5 mL) was purged with nitrogen, stirred at 90° C. for 24 h, cooled to R.T, diluted with EA (50 mL) and washed with water (2×30 mL). The organic layer was dried over Na₂SO₄ and concentrated under reduced pressure to obtain a residue which was purified with Pre-TLC (EA:Hex=1:5, v/v) to afford Compound 13-1 (91 mg, 142.96 μmol, 33.7% yield). MS m/z: 636 [M+H]⁺.

A mixture of Compound 13-1 (91 mg, 142.96 μmol), INT C1 (116 mg, 234.55 μmol), cataCXium A Pd G₃ (20 mg, 27.46 μmol), Cs₂CO₃ (158 mg, 484.93 μmol), toluene (8 mL) and water (2.0 mL) was stirred at 100° C. for 6 hrs under nitrogen atmosphere, diluted with EA (100 mL) and then washed with water (2×40 mL). The organic layer was dried over Na₂SO₄ and concentrated under reduced pressure to obtain a residue which was purified with Pre-TLC (MeOH:DCM=1:20, v/v) to afford Compound 13-2 (67 mg, 72.49 μmol, 50.7% yield). MS m/z: 924 [M+H]⁺.

TFA (1 mL) was added to a solution of Compound 13-2 (67 mg, 72.49 μmol) in DCM (5 mL). The reaction mixture was stirred at R.T for 1 h, and then concentrated under reduced pressure to obtain a crude product containing Compound 13-3 which was used in next step directly without purification.

A mixture of the crude product containing Compound 13-3, CsF (0.18 g, 1.18 mmol) and DMF (5 mL) was stirred at 40° C. for 16 hrs, and then concentrated under reduced pressure to obtain a residue which was purified with Pre-HPLC (Daisogel-C18 column, 50*250 mm, 10 μm; eluent A: 0.1% TFA in water; eluent B: CH₃CN; gradient: 15% B to 40% B in 40 min at a flow rate of 60 mL/min; 240 nm) to afford a TFA salt of Compound 13 (27.2 mg, 36.87 μmol, 50.8% yield). MS m/z: 624 [M+H]⁺.

The enantio separation of the Compound 13 was performed by chiral-HPLC with the following condition: CHIRAL ART Cellulose-SA column (2 cm×25 cm, 5 um) on Prep-HPLC-Gilson; Mobile phase: Hex (0.1% IPA.M)/EtOH (50:50); Flow rate: 20 mL/min. This resulted in a first eluting stereoisomer (Compound 13A, 5 mg, Retention Time 4.297 min) and a second eluting stereoisomer (Compound 13B, 4.8 mg, Retention Time 7.337 min).

### Example 14

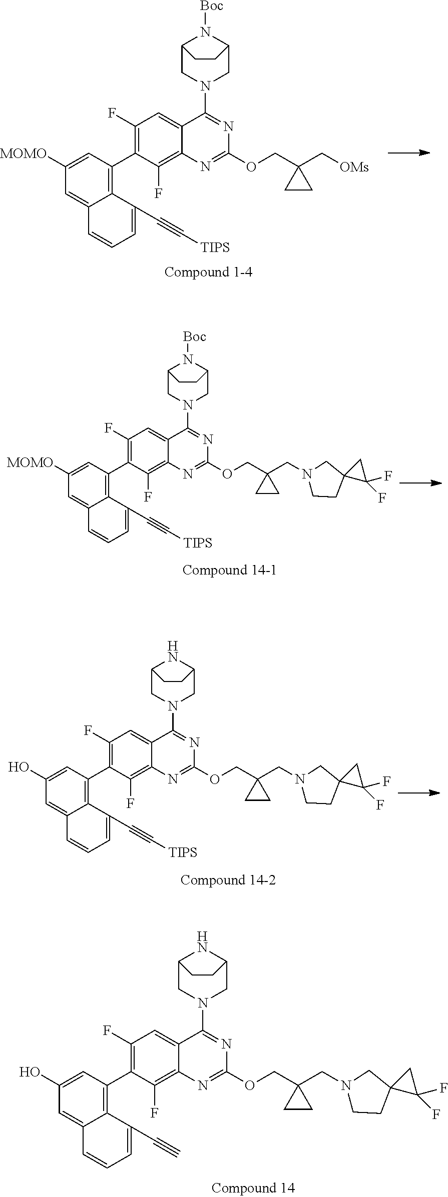

A mixture of 1,1-difluoro-5-azaspiro[2.4]heptane (55 mg, 0.3 mmol, 1.5 eq), TEA (102 mg, 1.0 mmol, 5.0 eq) and DMF (5 mL) was stirred at R.T for 2 hrs, and then Compound 1-4 (200 mg, 0.2 mmol, 1.0 eq.) was added. The reaction mixture was stirred for 2 hrs at 85° C., cooled to R.T, poured into water (5 mL) and then extracted with EtOAc (5 mL×2). The organic layers were combined, washed with water (5 mL) and brine (5 mL) successively, dried with anhydrous Na₂SO₄, and then filtered. The filtrate was concentrated to obtain a residue which purified with Pre-TLC (eluting with DCM/MeOH=10:1) to afford Compound 14-1 (48 mg) as a yellow oil. LCMS: 958.4 ([M+H]⁺).

A solution of Compound 14-1 (50 mg, 0.05 mmol, 1.0 eq) and HCl/1,4-Dioxane (2.5 mL) in MeCN (5 mL) was stirred for 1 h at 30° C., and then concentrated to afford a crude product containing Compound 14-2 (50 mg) as a yellow solid. LCMS: 814 ([M+H]⁺).

CsF (80 mg, 0.5 mmol, 10.0 eq) was added to a solution of the crude product containing Compound 14-2 (50 mg crude, 0.05 mmol, 1.0 eq) in DMF (3 mL) at R.T. The reaction mixture was stirred for 1 h at 45° C., and then filtered. The filtrate was purified with Prep-HPLC to afford Compound 14 (5 mg, the yield of the above three steps is 2.8%) as a white solid.

¹H NMR (400 MHz, CD₃OD-d₄): δ 7.80 (d, J=8.0 Hz, 1H), 7.50-7.47 (m, 2H), 7.40-7.36 (m, 1H), 7.31 (d, J=2.4 Hz, 1H), 7.06 (d, J=2.4 Hz, 1H), 4.51-4.42 (m, 2H), 4.39-4.29 (m, 1H), 3.89-3.87 (m, 2H), 3.4-3.63 (m, 2H), 2.20-2.85 (m, 4H), 2.4-2.61 (m, 3H), 2.14-2.09 (m, 1H), 2.00-1.99 (m, 4H), 1.38-1.28 (m, 4H), 0.6-0.71 (m, 2H), 0.56-0.55 (m, 2H). LCMS: 658.0 ([M+H]⁺).

### Example 15

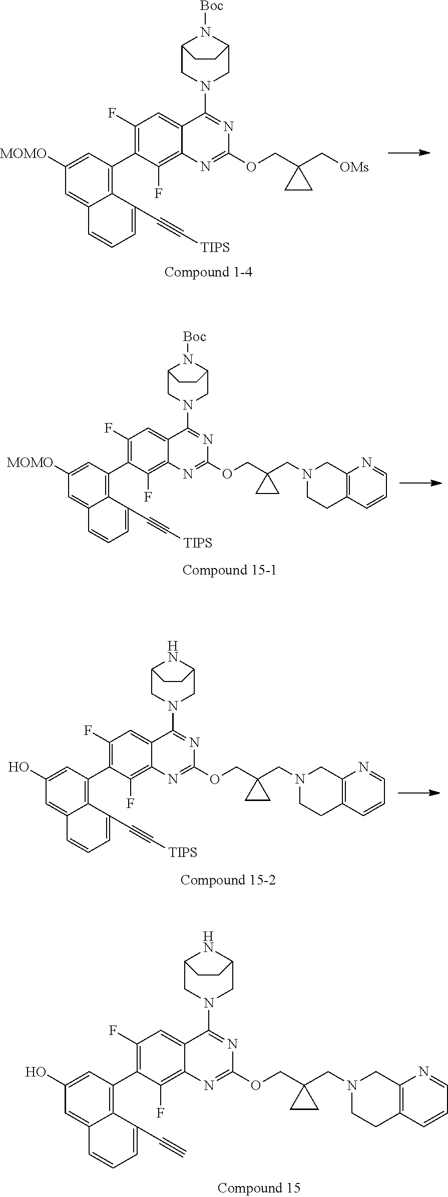

5,6,7,8-tetrahydro-1,7-naphthyridine (48.3 mg, 0.36 mmol, 1.5 eq) and TEA (48.6 mg, 0.48 mmol, 2.0 eq) were added to a solution of Compound 1-4 (220.0 mg, 0.24 mmol, 1.0 eq) in DMF (4 mL) at R.T. The reaction mixture was stirred for 2 hrs at 85° C., poured into water (10 mL) and then extracted with EtOAc (15 mL×2). The organic layers were combined, washed with brine (10 mL), dried over Na₂SO₄, and then filtered. The filtrate was concentrated to obtain a residue which was purified with Prep-TLC (eluting with petroleum ether/EtOAc=1:1) to afford Compound 15-1 (50.0 mg, 21.8%) as a yellow solid. LCMS: 959 ([M+H]⁺).

HCL/Dioxane (4M 1 mL) was added to the solution of Compound 15-1 (50.0 mg, 0.05 mmol, 1.0 eq) in MeCN (2 mL) at 0° C. The reaction mixture was stirred at R.T for 1 h, and then concentrated to afford a crude product containing Compound 15-2 (60.0 mg) as a yellow solid. LCMS: 815.40 ([M+H]⁺).

CsF (28.8 mg, 0.2 mmol, 10.0 eq) was added to a solution of Compound 15-2 (50.0 mg crude, 0.02 mmol, 1.0 eq) in DMF (1 mL) at R.T. The reaction mixture was stirred at R.T for 3 hrs, and then filtered. The filtrate was purified with Prep-HPLC to afford Compound 15 (9.2 mg, the yield of the above two steps is 26.8%) as a white solid.

¹H NMR (400 MHz, CD₃OD-d₄): δ 8.27 (d, J=4.0 Hz, 1H), 7.81 (d, J=8.0 Hz, 1H), 7.51-7.48 (m, 2H), 7.42-7.38 (m, 2H), 7.31 (d, J=2.4 Hz, 1H), 7.18-7.17 (m, 1H), 7.06 (d, J=2.4 Hz, 1H), 4.53-4.40 (m, 4H), 4.03 (s, 2H), 3.75-3.66 (m, 4H), 2.19-2.93 (m, 5H), 2.4-2.66 (m, 2H), 2.09-2.02 (m, 4H), 0.80-0.79 (m, 2H), 0.61-0.60 (m, 2H). LCMS: 659.0 ([M+H]⁺).

### Example 16

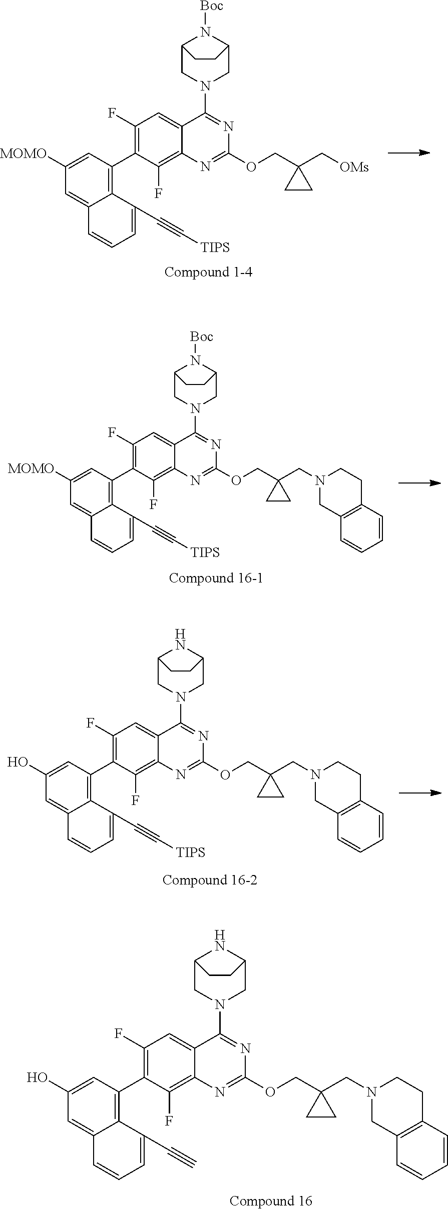

TEA (102 mg, 1.0 mmol, 5.0 eq) was added to the solution of 1,2,3,4-tetrahydroisoquinoline (43 mg, 0.3 mmol, 1.5 eq) in DMF (5 mL), and then Compound 1-4 (200 mg, 0.2 mmol, 1.0 eq) was added. The reaction mixture was stirred for 2 hrs at 85° C., cooled to R.T, poured into water (5 mL) and extracted with EtOAc (5 mL×2). The organic layers were combined, washed with water (5 mL) and brine (5 mL) successively, dried with anhydrous Na₂SO₄, and then filtered. The filtrate was concentrated to obtain a residue which was purified with Pre-TLC (eluting with petroleum ether/EtOAc=1:1) to afford Compound 16-1 (80 mg) as a yellow liquid. LCMS: 958.4 ([M+H]⁺).

HCl/Dioxane (4M, 2.5 mL) was added to a solution of Compound 16-1 (80 mg, 0.08 mmol, 1.0 eq) in MeCN (5 mL) at R.T. The reaction mixture was stirred for 1 h, and then concentrated to afford a crude product containing Compound 16-2 (80 mg) as a yellow solid. LCMS: 814.1 ([M+H]⁺).

CsF (152 mg, 1.0 mmol, 10.0 eq) was added to a solution of the crude product containing Compound 16-2 (80 mg) in DMF (3 mL) at R.T. The reaction mixture was stirred at R.T for 14 hrs, and then filtered. The filtrate was purified with Prep-HPLC to afford Compound 16 (13 mg, the yield of the above three steps is 2.8%) as a black solid. LCMS: 658.3 ([M+H]⁺).

### Example 17

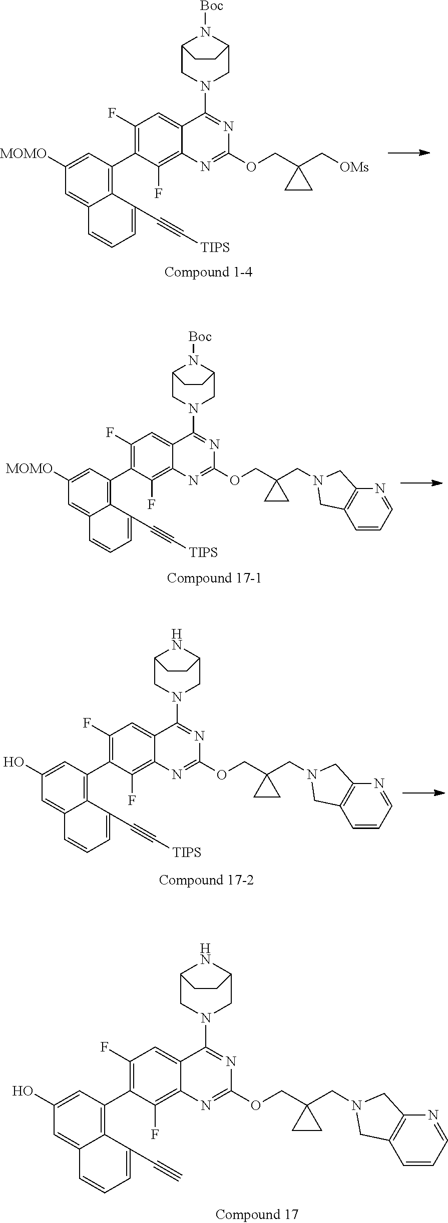

TEA (110 mg, 1.0 mmol, 5.0 eq) was added to a solution of 6,7-dihydro-5H-pyrrolo[3,4-b]pyridine (39 mg, 0.3 mmol, 1.5 eq) in DMF (5 mL), and then Compound 1-4 (200 mg, 0.2 mmol, 1.0 eq) was added. The reaction mixture was stirred for 2 hrs at 85° C., cooled to R.T, poured into water (5 mL) and extracted with EtOAc (5 mL×2). The organic layers were combined, washed with water (5 mL) and brine (5 mL) successively, dried with anhydrous Na₂SO₄, and then filtered. The filtrate was concentrated to obtain a residue which was purified with Pre-TLC (eluting with petroleum ether/EtOAc=2:1) to afford Compound 17-1 (72 mg, yield 35.1%) as a yellow liquid. LCMS: 945.5 ([M+H]⁺).

HCl/1,4-dioxane (2.5 mL) was added to a solution of Compound 17-1 (70 mg, 0.07 mmol, 1.0 eq) in MeCN (5 mL) at 30° C. The reaction mixture was stirred for 1 h, and then concentrated to afford a crude product containing Compound 17-2 (75 mg) as a yellow solid. LCMS: 801.4 ([M+H]⁺).

CsF (152 mg, 1.0 mmol, 10.0 eq) was added to a solution of the crude product containing Compound 17-2 (80 mg) in DMF (3 mL) at R.T. The reaction mixture was stirred at R.T for 14 hrs, and then filtered. The filtrate was purified with Prep-HPLC to afford Compound 17 (3 mg, the yield of the above two steps is 4.9%) as a white solid.

¹H NMR (400 MHz, CD₃OD-d₄): δ 8.51-8.52 (m, 1H), 7.80 (d, J=8.0 Hz, 1H), 7.64 (d, J=7.8 Hz, 1H), 7.49-7.47 (m, 1H), 7.44-7.36 (m, 2H), 7.36 (d, J=2.4 Hz, 1H), 7.23-7.20 (m, 1H), 7.05-7.04 (m, 1H), 4.52-4.49 (m, 1H), 4.43-4.39 (m, 1H), 4.04-3.98 (m, 4H), 3.90-3.87 (m, 2H), 3.63 (t, J=13.6 Hz, 2H), 2.95 (s, 1H), 2.86 (q, J=12.8 Hz, 2H), 2.00-1.94 (m, 4H), 1.30-1.28 (m, 2H), 0.77-0.75 (m, 2H), 0.63-0.61 (m, 2H). LCMS: 645.3 ([M+H]⁺).

### Example 18

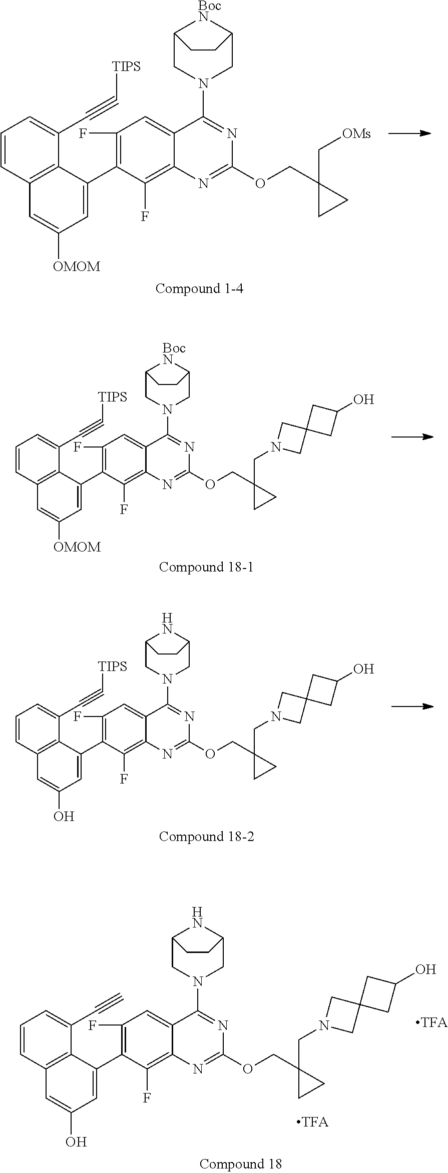

Compound 1-4 (150 mg, 0.16 mmol, 1.0 eq), 2-azaspiro[3.3]heptan-6-ol hydrogen chloride (28.7 mg, 0.19 mmol, 1.2 eq), NaHCO₃ (37.8 mg, 0.48 mmol, 3.0 eq), KI (29.7 mg, 0.208 mmol, 1.3 eq) were added successively into DMF (2.1 mL). The reaction mixture was stirred at 85° C. or 3 hrs, cooled to R.T, poured into water (5 mL) and extracted with EtOAc (5 mL×2). The organic layers were combined, washed with brine (5 mL), dried with anhydrous Na₂SO₄, and then filtered. The filtrate was concentrated to obtain a residue which was purified with Pre-TLC (DCM/MeOH=20:1) to afford Compound 18-1 (42 mg, 0.045 mmol, 27.9% yield) as a yellow oil. LCMS: 938.4 ([M+H]⁺).

TFA (0.2 mL) was added to a solution of Compound 18-1 (32 mg, 0.045 mmol, 1.0 eq) in DCM (1.0 mL) at R.T. The reaction mixture was stirred at R.T for 1 h, and then concentrated to afford a crude product containing Compound 18-2 as a yellow solid which was used directly for the next step without any further purification. LCMS: 794 ([M+H]⁺).

CsF (50.1 mg, 0.33 mmol, 15.0 eq) was added to a solution of the crude product containing Compound 18-2 in DMF (1 mL). The reaction mixture was stirred overnight at 50° C., and then filtered. The filtrate was purified with Prep-HPLC to afford the TFA salt of Compound 18 (1.2 mg) as a white solid. LCMS: 638.3 ([M+H]⁺).

### Example 19

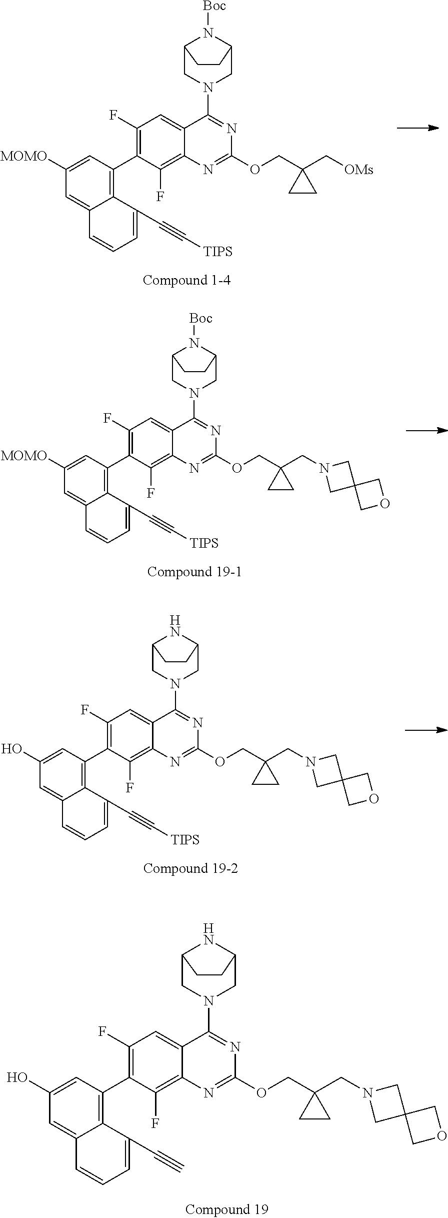

2-oxa-6-azaspiro [3.3] heptane (32.0 mg, 0.33 mmol, 1.5 eq) and TEA (45.0 mg, 0.44 mmol, 2.0 eq) was added to a solution of Compound 1-4 (200.0 mg, 0.22 mmol, 1.0 eq) in DMF (4 mL) at R.T. The reaction mixture was stirred for 2 hrs at 85° C., poured into water (10 mL) and then extracted with EtOAc (15 mL×2). The organic layers were combined, washed with brine (10 mL), dried over Na₂SO₄, and then filtered. The filtrate was concentrated to obtain a residue which was purified with Prep-TLC (eluting with petroleum ether/EtOAc=1:1) to afford Compound 19-1 (35.0 mg, 17.4%) as a yellow solid. LCMS: 924 ([M+H]⁺).

TFA (0.4 mL) was added to a solution of Compound 19-1 (35.0 mg, 0.04 mmol, 1.0 eq) in DCM (2 mL) at 0° C. The reaction mixture was stirred at R.T for 1 h and then diluted with DCM (4.0 mL). The pH of the resulting mixture was adjusted to 8 with saturated sodium bicarbonate. The organic layer was separated, washed with brine (10 mL), dried over Na₂SO₄, and then filtered. The filtrate was concentrated to afford a crude product containing Compound 19-2 (40.0 mg) as a yellow solid. LCMS: 780 ([M+H]⁺).

CsF (57.5 mg, 0.4 mmol, 10.0 eq) was added to a solution of the crude product containing Compound 19-2 (40.0 mg) in DMF (1 mL) at R.T. The reaction mixture was stirred for 3 hrs at R.T, and then filtered. The filtrate was purified with Prep-HPLC to afford Compound 19 (2.5 mg, the yield of the above two steps is 10.6%) as a white solid. ¹H NMR (300 MHz, CD₃OD-d₄): δ 7.79 (dd, J=8.4 Hz, 1.2 Hz, 1H), 7.50-7.44 (m, 2H), 7.37-7.34 (m, 1H), 7.29 (d, J=2.7 Hz, 1H), 7.05 (d, J=2.7 Hz, 1H), 4.2-4.68 (m, 4H), 4.46-4.36 (m, 2H), 4.25 (q, J=9.0 Hz, 2H), 3.61-3.51 (m, 4H), 3.45-3.44 (m, 4H), 2.95 (s, 1H), 2.54 (s, 2H), 1.89-1.85 (m, 4H), 0.61-0.51 (m, 4H). LCMS: 624.3 ([M+H]⁺).

### Example 20

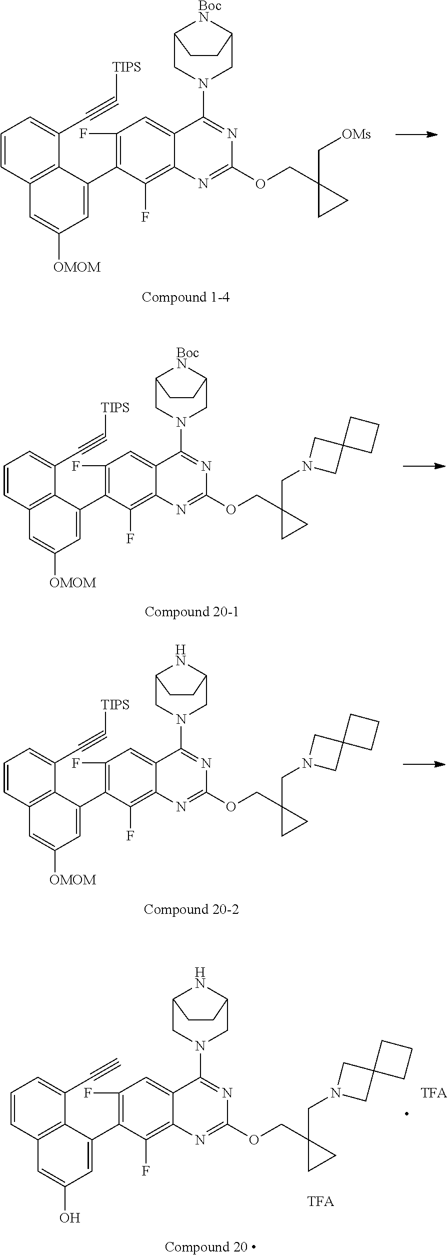

Compound 1-4 (150 mg, 0.16 mmol, 1.0 eq), 2-azaspiro[3.3]heptane oxalic acid (55.5 mg, 0.19 mmol, 1.2 eq), NaHCO₃ (37.8 mg, 0.48 mmol, 3.0 eq), KI (29.7 mg, 0.208 mmol, 1.3 eq) were added successively into DMF (2.1 mL). The reaction mixture was stirred at 85° C. for 3 hrs, cooled to R.T, poured into water (5 mL) and extracted with EtOAc (5 mL×2). The organic layers were combined, washed with brine (5 mL), dried with anhydrous Na₂SO₄, and then filtered. The filtrate was concentrated to obtain a residue which was purified with Pre-TLC (DCM/MeOH=10:1) to afford Compound 20-1 (74 mg, 50.1% yield) as a yellow oil. LCMS: 922.4 ([M+H]⁺).

A solution of Compound 20-1 (74 mg, 0.08 mmol, 1.0 eq) and HCl/Dioxane (0.6 mL) in MeCN (2.7 mL) was stirred at R.T for 1 h, and then concentrated under reduced pressure to afford a crude product containing Compound 20-2 as a yellow solid which was used directly for the next step without any further purification. LCMS: 778.4 ([M+H]⁺).

CsF (182.3 mg, 1.2 mmol, 15.0 eq) was added to a solution of the crude product containing Compound 20-2 in DMF (3 mL). The reaction mixture was stirred overnight at 50° C., and then filtered. The filtrate was purified with Prep-HPLC to afford a TFA salt of Compound 20 (9.3 mg) as a white solid.

¹H NMR (300 MHz, DMSO-d₆): δ 10.18 (s, 1H), 8.17 (s, 1H), 7.90 (dd, J=7.9, 1.6 Hz, 1H), 7.57-7.39 (m, 3H), 7.36 (d, J=2.5 Hz, 1H), 7.07 (d, J=2.5 Hz, 1H), 4.25 (d, J=7.9 Hz, 2H), 4.13 (s, 2H), 3.64 (s, 2H), 3.61-3.60 (m, 1H), 3.55 (s, 1H), 3.51 (s, 2H), 3.47 (s, 1H), 2.45 (s, 2H), 2.02 (t, J=7.5 Hz, 6H), 1.78-1.68 (m, 6H), 0.52-0.51 (m, 2H), 0.44-0.43 (m, 2H). LCMS: 622.3 ([M+H]⁺).

### Example 21

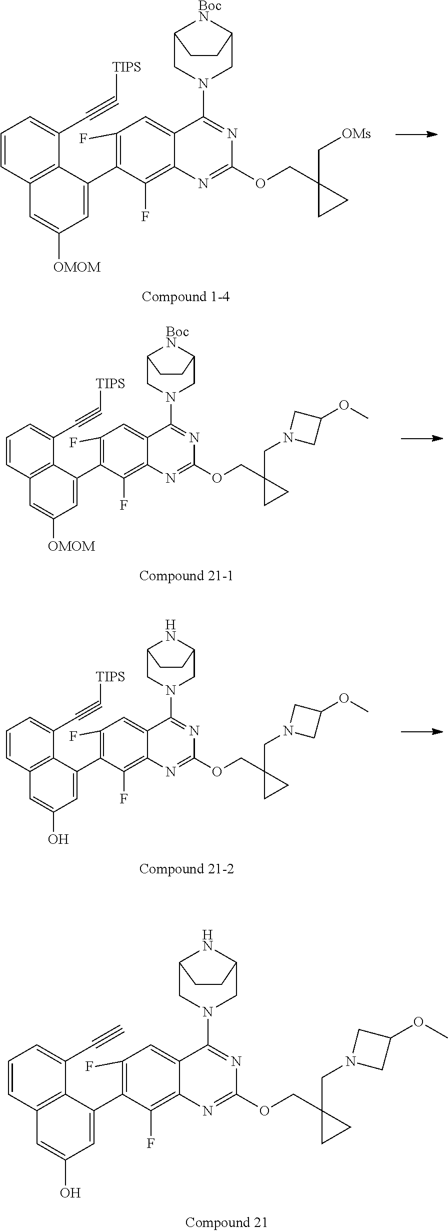

A mixture of 3-methoxyazetidine hydrogen chloride (39.5 mg, 0.32 mmol, 2.0 eq), TEA (81.0 mg, 0.85 mmol, 5.0 eq) and DMF (1.5 mL) was stirred at room temperature for 2 hrs, and then Compound 1-4 (150.0 mg, 0.16 mmol, 1.0 eq) was added. The reaction mixture was stirred for 2 hrs at 85° C., cooled to R.T, poured into water (5 mL) and extracted with EtOAc (5 mL×2). The organic layers were combined, washed with brine (5 mL), dried with anhydrous Na₂SO₄, and then filtered. The filtrate was concentrated to obtain a residue which was purified with Pre-TLC (DCM/MeOH=10:1) to afford Compound 21-1 (27 mg, 0.03 mmol, 18.5% yield) as a yellow oil. LCMS: 912.4 ([M+H]⁺).

A solution of Compound 21-1 (27 mg, 0.03 mmol, 1.0 eq), HCl/Dioxane (0.23 mL) and MeCN (0.9 mL) was stirred at R.T for 1 h, and then concentrated under reduced pressure to afford a crude product containing Compound 21-2 as a yellow solid which was used directly for the next step without any further purification. LCMS: 768.4 ([M+H]⁺).

CsF (68.4 mg, 0.45 mmol, 15.0 eq) was added to a solution of the crude product containing Compound 21-2 in DMF (1 mL). The reaction mixture was stirred overnight at 50° C., and then filtered. The filtrate was purified with Prep-HPLC to afford Compound 21 (3.7 mg) as a white solid.

¹H NMR (300 MHz, DMSO-d₆): δ 10.19 (s, 1H), 7.89 (d, J=8.0 Hz, 1H), 7.47 (ddd, J=19.2, 14.8, 8.6 Hz, 3H), 7.36 (d, J=2.6 Hz, 1H), 7.08 (d, J=2.5 Hz, 1H), 4.24 (d, J=11.5 Hz, 2H), 4.13 (s, 2H), 3.92 (d, J=5.8 Hz, 1H), 3.65-3.42 (m, 7H), 3.11 (s, 3H), 2.83-2.74 (m, 2H), 2.29-2.24 (m, 1H), 2.02-1.96 (m, 2H), 1.73 (d, J=21.9 Hz, 4H), 0.51-0.50 (m, 2H), 0.42-0.41 (m, 2H). LCMS: 612.3 ([M+H]⁺).

### Example 22

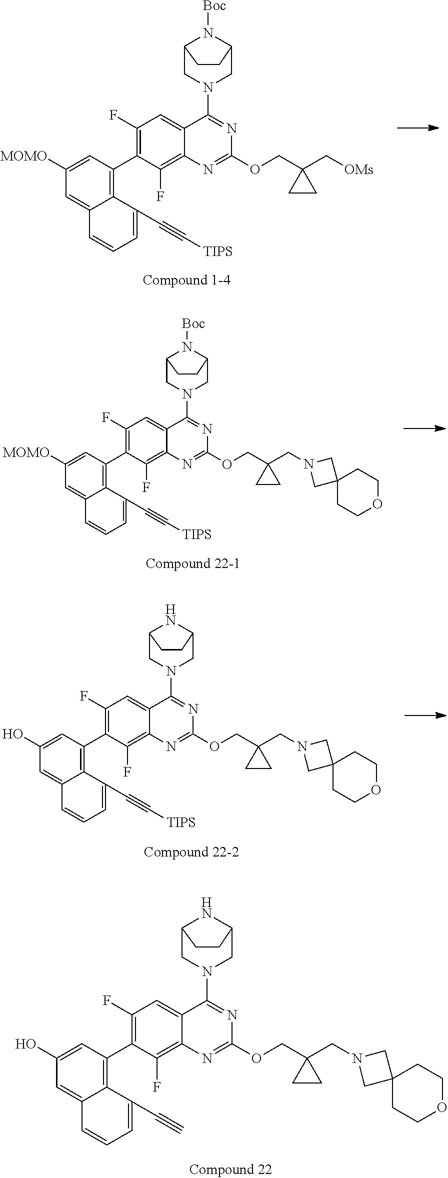

TEA (132 mg, 1.3 mmol, 5.0 eq) and Compound 1-4 (240 mg, 0.26 mmol, 1.0 eq) were added successively to a solution of 7-oxa-2-azaspiro [3.5] nonane (50 mg, 0.39 mmol, 1.5 eq.) in DMF (5 mL). The reaction mixture was stirred for 2 hrs at 85° C., cooled to R.T, poured into water (5 mL) and then extracted with EtOAc (5 mL×2). The organic layers were combined, washed with water (5 mL) and brine (5 mL) successively, dried with anhydrous Na₂SO₄, and then filtered. The filtrate was concentrated to obtain a residue which was purified with Pre-TLC (eluting with DCM/MeOH=10:1) to afford Compound 22-1 (72 mg, yield 34.5%) as a yellow solid. LCMS: 952.4 ([M+H]⁺).

A solution of HCl/Dioxane (4M, 2.5 mL) and Compound 22-1 (100 mg, 0.1 mmol, 1.0 eq.) in MeCN (5 mL) was stirred at R.T for 1 h, and then concentrated to afford a crude product containing Compound 22-2 (110 mg) as a yellow solid. LCMS: 808.4 ([M+H]⁺).

CsF (152 mg, 1 mmol, 10.0 eq) was added to a solution of the crude product containing Compound 22-2 (110 mg) in DMF (3 mL) at R.T. The reaction mixture was stirred for 3 hrs at 50° C., and then filtered. The filtrate was purified with Prep-HPLC to afford Compound 22 (34.5 mg, the yield of the above two steps is 42.8%) as a white solid.

¹H NMR (400 MHz, CD₃OD-d₄): δ 7.80 (d, J=8.0 Hz, 1H), 7.50-7.47 (m, 2H), 7.40-7.36 (m, 1H), 7.30 (d, J=2.4 Hz, 1H), 7.11 (d, J=2.4 Hz, 1H), 4.47-4.33 (m, 3H), 4.27-4.24 (m, 1H), 3.65-3.60 (m, 3H), 3.57-3.54 (m, 5H), 3.49-3.47 (m, 4H), 2.96 (s, 1H), 2.87 (brs, 2H), 1.90-1.86 (m, 4H), 1.78-1.75 (m, 4H), 0.4-0.69 (m, 2H), 0.65-0.63 (m, 2H). LCMS: 652.3 ([M+H]⁺).

### Example 23

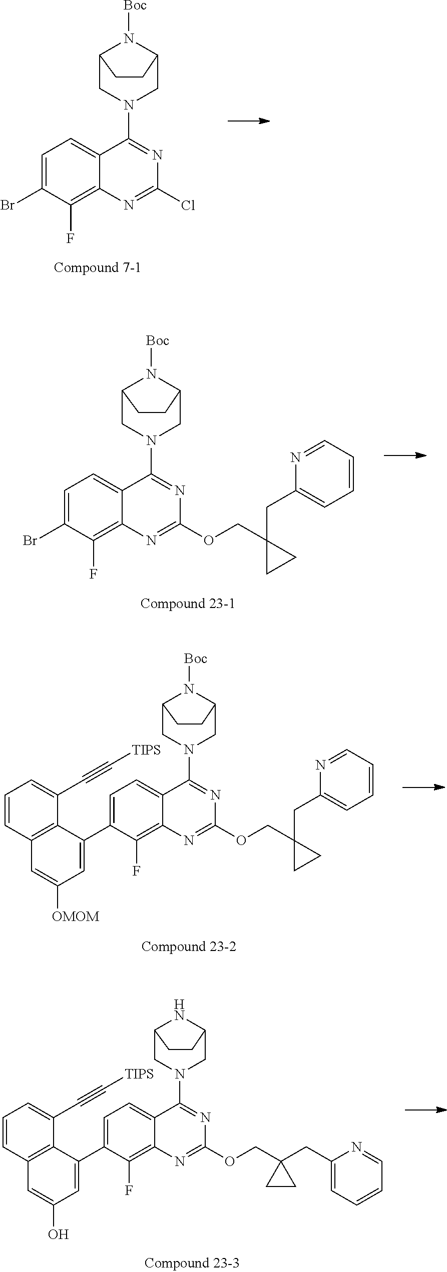

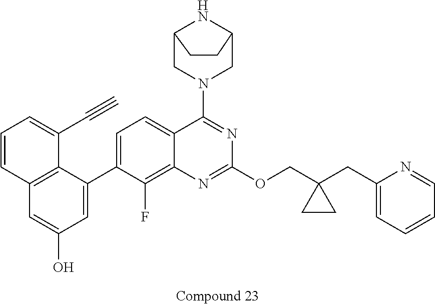 [SMILES: C#Cc1cccc2cc(O)cc(-c3ccc4c(N5CC6CCC(C5)N6)nc(OCC5(Cc6ccccn6)CC5)nc4c3F)c12]

A solution of INT B17 (207.6 mg, 1.3 mmol, 2.0 eq) in toluene (5 mL) was added dropwise to a suspension of NaH (60%, 72.0 mg, 1.8 mmol, 3.0 eq) in toluene (5 mL) at 0° C. The mixture was stirred at R.T for 1 h, and then Compound 7-1 (300.0 mg, 0.6 mmol, 1.0 eq) was added. The reaction mixture was stirred for 2 hrs at R.T, poured into ice/water and extracted with EtOAc (20 mL×2). The organic layers were combined, washed with brine (10 mL), dried over Na₂SO₄, and then filtered. The filtrate was concentrated to obtain a residue which was purified with Prep-TLC (eluting with petroleum ether/EtOAc=1:1) to afford Compound 23-1 (200.0 mg, 47.8%) as a yellow solid. LCMS: 597.8, 598.9 ([M+H]⁺).

Compound 23-1 (200 mg, 0.33 mmol, 1.0 eq), INT C1 (244.8 mg, 0.5 mmol, 1.5 eq), K₃PO₄ (210.1 mg, 1.0 mmol, 3.0 eq) and CataCxium A Pd G₃ (36.1 mg, 0.05 mmol, 0.15 eq) were added successively into a mixed solution of THF and H₂O (V_{THF}/V_{H2O}=1:1, 4 mL). The reaction mixture was purged with argon for 10 mins, stirred for 3 hrs at 60° C. under an atmosphere of argon, cooled to R.T, poured into water (5 mL) and extracted with DCM (10 mL×2). The organic layers were combined, washed with water (10 mL) and brine (10 mL) successively, dried over Na₂SO₄, and then filtered. The filtrate was concentrated to obtain a residue which was purified with Prep-TLC (eluting with EtOAc) to afford Compound 23-2 (200.0 mg, 67.6%) as a yellow solid. LCMS: 886 ([M+H]⁺).

TFA (1.0 mL) was added to a solution of Compound 23-2 (200.0 mg, 0.2 mmol, 1.0 eq) in DCM (5 mL) at 0° C. The mixture was stirred at R.T for 1 h, and then the pH of the reaction mixture was adjusted to 8 with saturated sodium bicarbonate. The resulting mixture was extracted with DCM (10 mL). The organic layer was washed with water (10 mL) and brine (10 mL) successively, dried over Na₂SO₄, and concentrated to afford a crude product containing Compound 23-3 (150.0 mg) as a yellow solid. LCMS: 742 ([M+H]⁺).

CsF (307.0 mg, 2.0 mmol, 10.0 eq) was added to a solution of the crude product containing Compound 23-3 (150.0 mg) in DMF (4 mL) at R.T. The reaction mixture was stirred for 3 hrs at 50° C., then filtered, and the filtrate was purified by Prep-HPLC to afford Compound 23 (13.4 mg, the yield of the above two steps is 10.1%) as a white solid.

¹H NMR (400 MHz, CD₃OD): δ 8.40 (d, J=4.8 Hz, 1H), 7.80-7.77 (m, 1H), 7.74-7.71 (m, 2H), 7.49-7.47 (m, 2H), 7.46-7.45 (m, 1H), 7.39-7.35 (m, 3H), 7.03 (s, 1H), 4.48-4.47 (m, 2H), 4.26-4.25 (m, 2H), 3.96 (s, 2H), 3.66-3.63 (m, 2H), 3.03 (s, 2H), 2.89 (s, 1H), 2.03-2.02 (m, 4H), 0.05-0.04 (m, 4H). LCMS: 586.20 ([M+H]⁺).

### Example 24

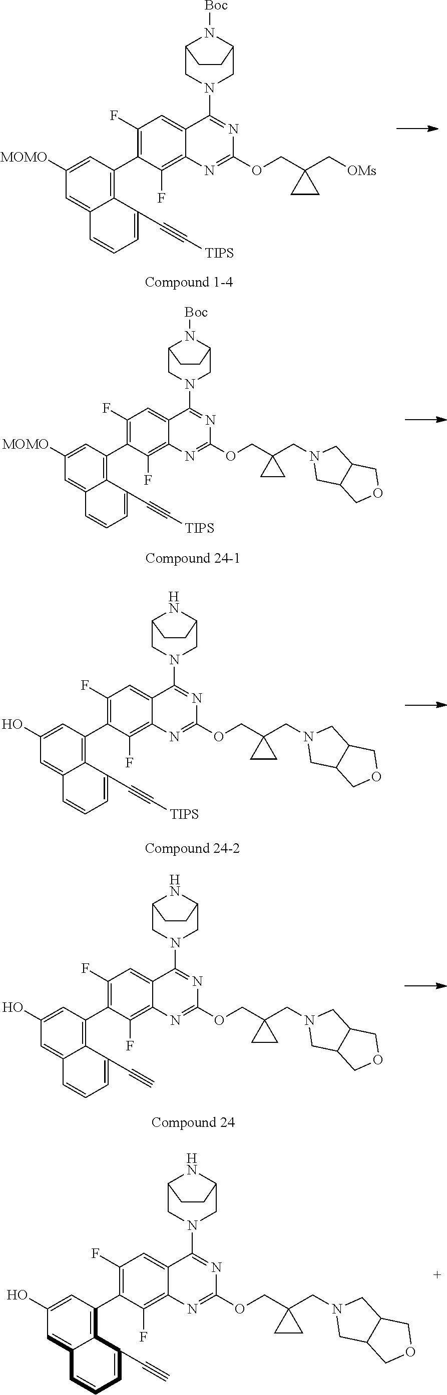

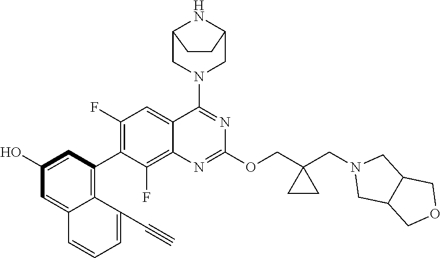

TEA (110 mg, 1.0 mmol, 5.0 eq) and Compound 1-4 (200 mg, 0.2 mmol, 1.0 eq) were added successively to a solution of Hexahydro-1H-furo[3,4-c] pyrrole (49 mg, 0.3 mmol, 1.5 eq) in DMF (3 mL). The reaction mixture was stirred for 2 hrs at 85° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which purified with Pre-TLC (eluting with DCM/MeOH=10:1) to afford Compound 24-1 (72 mg, yield 35.3%) as a yellow liquid. LCMS: 938.5 ([M+H]⁺).

A solution of Compound 24-1 (90 mg, 0.09 mmol, 1.0 eq) and HCl/Dioxane (4M. 2.5 mL) in MeCN (5 mL) was stirred for 1 h at 30° C., and then concentrated to afford a crude product containing Compound 24-2 (80 mg) as a yellow solid. LCMS: 794 ([M+H]⁺).

CsF (152 mg, 1.0 mmol, 10.0 eq) was added to a solution of the crude product containing Compound 24-2 (80 mg) in DMF (3 mL) at R.T. The reaction mixture was stirred for 3 hrs at 50° C., and then filtered. The filtrate was purified with Prep-HPLC to afford Compound 24 (16 mg, 26.3%) as a white solid.

¹H NMR (400 MHz, CD₃OD-d₄): δ 7.80 (d, J=8.0 Hz, 1H), 7.50-7.45 (m, 2H), 7.40-7.36 (m, 1H), 7.30 (d, J=2.4 Hz, 1H), 7.06 (d, J=2.4 Hz, 1H), 4.45-4.34 (m, 4H), 3.77-3.75 (m, 2H), 3.64-3.48 (m, 6H), 2.96 (s, 1H), 2.78-2.76 (m, 4H), 2.58-2.49 (m, 2H), 2.44-2.42 (m, 2H), 1.90-1.87 (m, 4H), 0.3-0.67 (m, 2H), 0.51-0.49 (m, 2H). LCMS: 638.3 ([M+H]⁺).

The enantio separation of the Compound 24 was performed by chiral-HPLC with the following condition: CHIRAL ART Amylose-SA column on Prep-HPLC-Gilson (2 cm×25 cm, 5 um); Mobile phase: Hex (0.1% IPA.M)/EtOH (50:50); Flow rate: 20 mL/min. This resulted in a first eluting stereoisomer (Compound 24A, 2.1 mg, retention time 4.597 min) and a second eluting stereoisomer (Compound 24B, 2.2 mg, retention time 7.228 min).

### Example 25

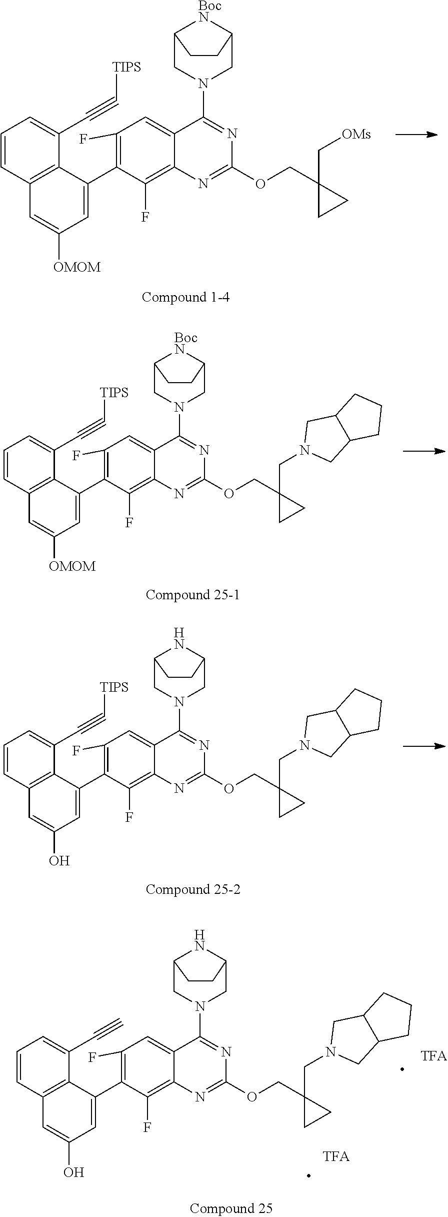

TEA (80.9 mg, 0.8 mmol, 5.0 eq.) was added to the solution of octahydrocyclopenta[c]pyrrole hydrogen chloride (47.2 mg, 0.32 mmol, 2.0 eq) in DMF (1.5 mL). The mixture was stirred for 2 hrs at R.T, and then Compound 1-4 (150.0 mg, 0.16 mmol, 1.0 eq) was added. The reaction mixture was stirred for 2 hrs at 80° C., cooled to R.T, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (DCM/MeOH=10:1) to afford Compound 25-1 (22 mg, 14.7% yield) as a yellow oil. LCMS: 936.4 ([M+H]⁺).

A solution of Compound 25-1 (22 mg, 0.023 mmol, 1.0 eq) and HCl/dioxane (4M, 0.2 mL) in MeCN (0.9 mL) was stirred at R.T for 1 h, and then concentrated under reduced pressure to afford a crude product containing Compound 25-2 as a yellow solid which was used directly for the next step without any further purification. LCMS: 792.4 ([M+H]⁺).

CsF (52.4 mg, 0.345 mmol, 15.0 eq) was added to a solution of the crude product containing Compound 25-2 in DMF (2 mL). The reaction mixture was stirred overnight at 50° C., and then filtered. The filtrate was purified with Prep-HPLC to afford Compound 25 (1.46 mg) as a white solid. LCMS: 636.3 ([M+H]⁺).

### Example 26

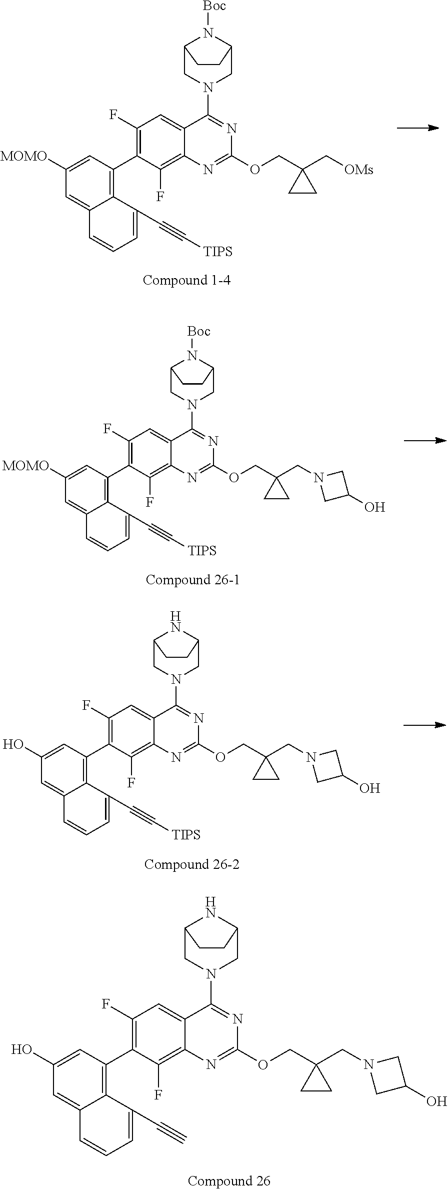

TEA (126.5 mg, 1.3 mmol, 5.0 eq) was added to a solution of azetidin-3-ol hydrochloride (41.0 mg, 0.37 mmol, 1.5 eq) in DMF (4 mL) at R.T. The mixture was stirred for 2 hrs, and then Compound 1-4 (230.0 mg, 0.25 mmol, 1.0 eq) was added. The reaction mixture was stirred for 2 hrs at 85° C., poured into water (10 mL) and extracted with EtOAc (15 mL×2). The organic layers were combined, washed with brine (10 mL), dried over Na₂SO₄, and concentrated to obtain a residue which was purified with Prep-TLC (eluting with petroleum ether/EtOAc=1:1) to afford Compound 26-1 (20.0 mg, 8.9%) as a yellow solid. LCMS: 898 ([M+H]⁺).

HCl/1,4-dioxane (4M, 1 mL) was added to a solution of Compound 26-1 (20.0 mg, 0.02 mmol, 1.0 eq) in MeCN (2 mL) at 0° C. The reaction mixture was stirred at R.T for 1 h, and then concentrated to afford a crude product containing Compound 26-2 (50.0 mg) as a yellow solid. LCMS: 754 ([M+H]⁺).

CsF (28.8 mg, 0.2 mmol, 10.0 eq) was added to a solution of the crude product containing Compound 26-2 (50.0 mg) in DMF (1 mL) at R.T. The reaction mixture was stirred for 3 hrs at 45° C., and then filtered. The filtrate was purified with Prep-HPLC to afford Compound 26 (2.5 mg, the yield of the above two steps is 16.8%) as a white solid.

¹H NMR (400 MHz, CD₃OD-d₄): δ 8.46 (s, 2H), 7.82 (d, J=8.4 Hz, 1H), 7.56-7.50 (m, 2H), 7.1-7.38 (m, 1H), 7.33 (d, J=2.4 Hz, 1H), 7.07 (d, J=2.4 Hz, 1H), 4.13-4.47 (m, 6H), 4.39-4.29 (m, 2H), 4.07-4.06 (m, 2H), 3.19-3.94 (m, 2H), 3.9-3.70 (m, 2H), 2.09-2.07 (m, 4H), 0.12-0.84 (m, 4H). LCMS: 598.20 ([M+H]⁺).

### Example 27

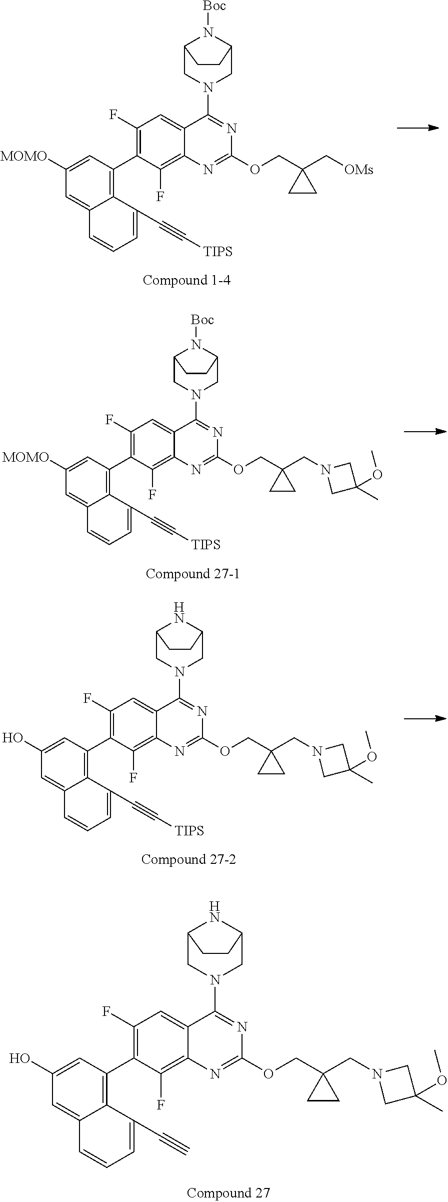

TEA (109 mg, 1.08 mmol, 5.0 eq) was added to a solution of 3-methoxy-3-methylazetidine hydrochloride (59.7 mg, 0.43 mmol, 2.0 eq.) in DMF (2 mL). The mixture was stirred for 2 hrs, and then Compound 1-4 (200 mg, 0.21 mmol, 1.0 eq) was added. The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with DCM/MeOH=10:1) to afford Compound 27-1 (45 mg, yield 19.8%) as a yellow oil. LCMS: 926 ([M+H]⁺).

A solution of Compound 27-1 (45 mg, 0.04 mmol, 1.0 eq), HCl/Dioxane (4M, 0.4 mL) in MeCN (2 mL) was stirred for 2 hrs at R.T, and then concentrated to afford a crude product containing Compound 27-2 (40 mg) as a yellow solid. LCMS: 782 ([M+H]⁺).

CsF (105 mg, 0.75 mmol, 15.0 eq) was added to a solution of the crude product containing Compound 27-2 (40 mg) in DMF (1 mL) at R.T. The reaction mixture was stirred for 3 hrs at 40° C., and then filtered. The filtrate was purified with Prep-HPLC to afford Compound 27 (6 mg, yield 22.0%) as a white solid.

¹H NMR (400 MHz, CD₃OD-d₄): δ 7.82 (d, J=8.0 Hz, 1H), 7.55-7.49 (m, 2H), 7.39 (t, J=7.6 Hz, 1H), 7.33-7.32 (m, 1H), 7.07 (d, J=2.4 Hz, 1H), 4.64-4.52 (m, 2H), 4.39-4.35 (m, 4H), 4.30-4.20 (m, 2H), 3.13-3.83 (m, 1H), 3.78-3.75 (m, 1H), 3.60-3.40 (m, 3H), 3.23-3.13 (m, 4H), 3.04 (s, 1H), 2.26-2.17 (m, 4H), 1.48 (s, 3H), 0.88-0.86 (m, 4H). LCMS: 626.30 ([M+H]⁺).

### Example 28

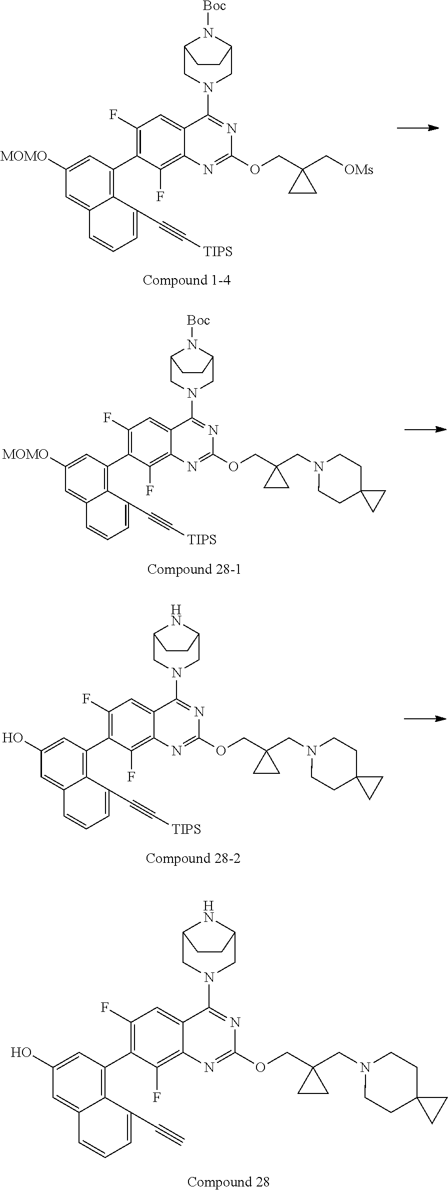

TEA (109 mg, 1.08 mmol, 5.0 eq) was added to a solution of 6-azaspiro [2.5] octane hydrochloride (64.1 mg, 0.43 mmol, 2.0 eq) in DMF (2 mL). The mixture was stirred for 2 hrs at R.T, and then Compound 1-4 (200 mg, 0.21 mmol, 1.0 eq) was added. The reaction mixture was stirred for 2 hrs at 80° C., cooled to R.T, poured in to water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with DCM/MeOH=10:1) to afford Compound 28-1 (40 mg, yield 19.8%) as a yellow oil. LCMS: 936 ([M+H]⁺).

A solution of Compound 28-1 (40 mg, 0.04 mmol, 1.0 eq) and HCl/dioxane (4 M, 0.4 mL) in MeCN (2 mL) was stirred for 2 hrs at R.T, and then concentrated to afford a crude product containing Compound 28-2 (40 mg) as a yellow solid. LCMS: 792 ([M+H]⁺).

CsF (115 mg, 0.75 mmol, 15.0 eq) was added to a solution of the crude product containing Compound 28-2 (40 mg) in DMF (1 mL) at R.T. The reaction mixture was stirred for 3 hrs at 40° C., and then filtered. The filtrate was purified with Prep-HPLC to afford Compound 28 (6 mg, yield 22.0%) as a white solid.

¹H NMR (400 MHz, CD₃OD-d₄): δ 7.82 (d, J=8.0 Hz, 1H), 7.45-7.59 (m, 2H), 7.39 (t, J=8.0 Hz, 1H), 7.32-7.31 (m, 1H), 7.06 (d, J=2.4 Hz, 1H), 4.53-4.41 (m, 3H), 4.35 (d, J=12 Hz, 1H), 3.8-3.76 (m, 2H), 3.2-3.59 (m, 3H), 3.20-3.10 (m, 4H), 2.97 (s, 1H), 1.18-1.92 (m, 5H), 1.68-1.64 (m, 3H), 1.32-1.28 (m, 1H), 0.15-0.93 (m, 2H), 0.78-0.81 (m, 2H), 0.43-0.41 (m, 4H). LCMS: 636.30 ([M+H]⁺).

### Example 29

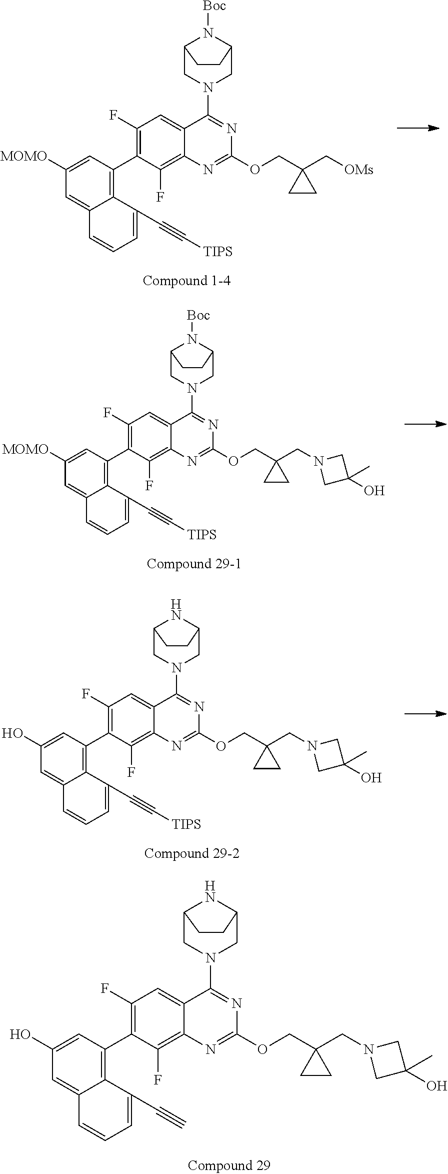

TEA (101 mg, 1.0 mmol, 5.0 eq) was added to a solution of 3-methylazetidin-3-ol (37 mg, 0.3 mmol, 1.5 eq) in DMF (3 mL). The mixture was stirred for 2 hrs at R.T, and then Compound 1-4 (184 mg, 0.2 mmol, 1.0 eq) was added. The reaction mixture was stirred for 2 hrs at 85° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 29-1 (40 mg, yield 21.9%) as a yellow liquid. LCMS: 912.4 ([M+H]⁺).

A solution of Compound 29-1 (50 mg, 0.05 mmol, 1.0 eq) and HCl/1,4-dioxane (2.5 mL) in MeCN (5 mL) was stirred for 1 h at 30° C., and then concentrated to afford a crude product containing Compound 29-2 (70 mg) as a yellow solid. LCMS: 768.4 ([M+H]⁺).

CsF (137 mg, 0.9 mmol, 10.0 eq) was added to a solution of the crude product containing Compound 29-2 (70 mg) in DMF (3 mL) at R.T. The reaction mixture was stirred for 3 hrs at 45° C., and then filtered. The filtrate was purified with Prep-HPLC to afford Compound 29 (2.4 mg, the yield of the above two steps is 7.2%) as a white solid.

¹H NMR (400 MHz, CD₃OD-d₄): δ 7.80 (d, J=8.0 Hz, 1H), 7.50-7.49 (m, 2H), 7.40-7.36 (m, 1H), 7.31 (d, J=2.4 Hz, 1H), 7.06 (d, J=2.4 Hz, 1H), 4.48-4.39 (m, 2H), 4.35-4.26 (m, 2H), 3.8-3.77 (m, 2H), 3.5-3.70 (m, 2H), 3.65-3.52 (m, 4H), 2.96 (s, 3H), 1.14-1.89 (m, 4H), 1.46 (s, 3H), 0.6-0.66 (m, 4H). LCMS: 612.3 ([M+H]⁺).

### Example 30

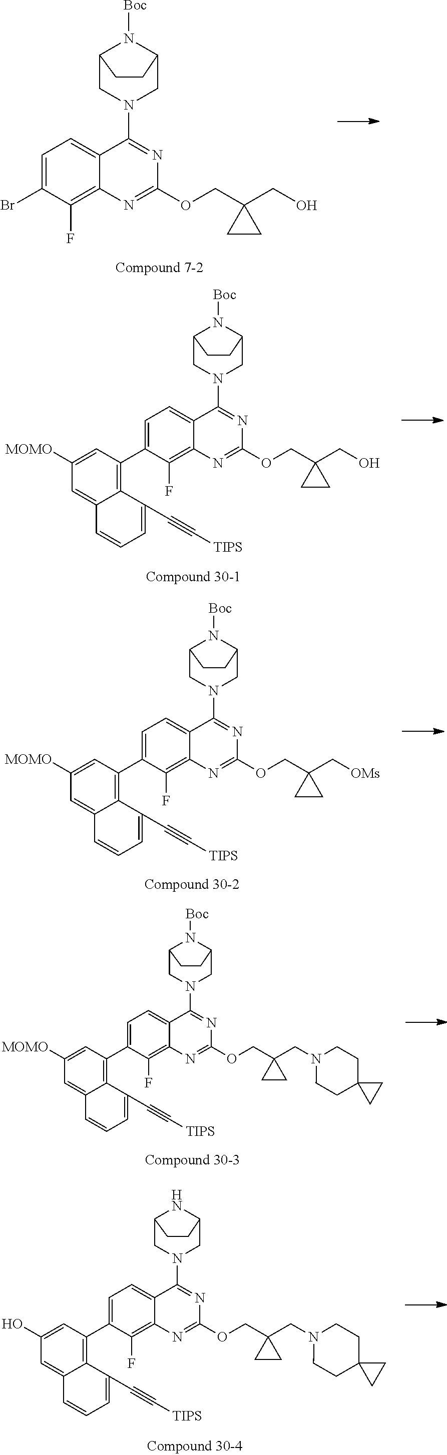

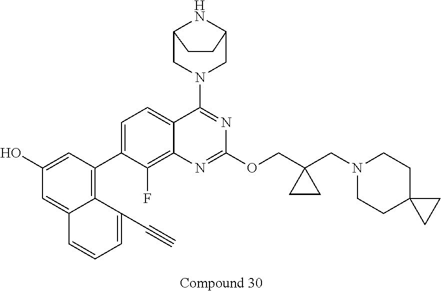 [SMILES: C#Cc1cccc2cc(O)cc(-c3ccc4c(N5CC6CCC(C5)N6)nc(OCC5(CN6CCC7(CC6)CC7)CC5)nc4c3F)c12]

Compound 7-2 (5.2 g, 9.7 mmol, 1.0 eq.), INT C1 (7.17 g, 14.5 mmol, 1.5 eq.), K₃PO₄ (6.15 g, 29.0 mmol, 3.0 eq.) and cataCxium A Pd G₃ (1.06 g, 1.45 mmol, 0.15 eq.) were added successively into a mixed solution of THF and water (V_{THF}/V_{water}=5:1, 60 mL) at R.T. The reaction mixture was purged with argon for 10 mins, stirred at 60° C. for 3 hrs, cooled to R.T, poured into water (60 mL) and extracted with EtOAc (60 mL×2). The organic layers were combined, washed with brine (30 mL), dried with anhydrous Na₂SO₄, and then concentrated to obtain a residue which was purified with silica gel chromatography (eluting with petroleum ether/EtOAc=3:1) to afford Compound 30-1 (3.2 g, yield 40.0%) as a yellow solid. LCMS: 825 ([M+H]⁺).

A mixture of Compound 30-1 (3.2 g, 3.88 mmol, 1.0 eq.), MsCl (533 mg, 4.65 mmol, 1.2 eq.), TEA (1.17 g, 11.6 mmol, 3.0 eq.) and DCM (32 mL) was stirred for 2 hrs at R.T, poured into water (30 mL) and extracted with DCM (20 mL). The organic layer was washed with brine (20 mL), dried with anhydrous Na₂SO₄, and concentrated to obtain a crude product containing Compound 30-2 (3.2 g, yield 91.3%) as a yellow solid which was used directly for next step without purification. LCMS: 903 ([M+H]⁺).

TEA (83.9 mg, 0.83 mmol, 5.0 eq) was added to a solution of 6-azaspiro [2.5] octane hydrochloride (49.1 mg, 0.33 mmol, 2.0 eq) in DMF (2 mL). The mixture was stirred for 2 hrs at R.T, and then Compound 30-2 (150 mg, 0.16 mmol, 1.0 eq) was added. The reaction mixture was stirred for 2 hrs at 80° C., cooled to R.T, poured into water (5 mL) and extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluted with DCM/MeOH=10:1) to afford Compound 30-3 (60 mg, yield 39.3%) as a yellow oil. LCMS: 918 ([M+H]⁺).

A solution of Compound 30-3 (60 mg, 0.06 mmol, 1.0 eq), HCl/1,4-dioxane (0.4 mL) in MeCN (2 mL) was stirred for 2 hrs at R.T, and then concentrated to afford a crude product containing Compound 30-4 (40 mg) as a yellow solid. LCMS: 774 ([M+H]⁺).

CsF (162 mg, 1.16 mmol, 15.0 eq) was added to a solution of the crude product containing Compound 30-4 (60 mg) in DMF (1 mL) at R.T. The reaction mixture was stirred for 3 hrs at 40° C., and then filtered. The filtrate was purified with Prep-HPLC to afford Compound 30 (6 mg, yield 14.8%) as a white solid.

¹H NMR (400 MHz, CD₃OD-d₄): δ 7.79 (t, J=8.0 Hz, 1H), 7.79 (d, J=7.2 Hz, 1H), 7.37 (t, J=7.6 Hz, 1H), 7.29-7.25 (m, 2H), 7.03 (d, J=2.52 Hz, 1H), 4.59-4.45 (m, 4H), 4.32 (d, J=10.8 Hz, 1H), 3.68-3.57 (m, 4H), 3.13-3.10 (m, 5H), 2.87 (s, 1H), 2.00-1.80 (m, 5H), 1.61-1.55 (m, 4H), 1.31-1.28 (m, 4H), 0.15-0.86 (m, 2H), 0.5-0.70 (m, 2H). LCMS: 618.30 ([M+H]⁺).

### Example 31

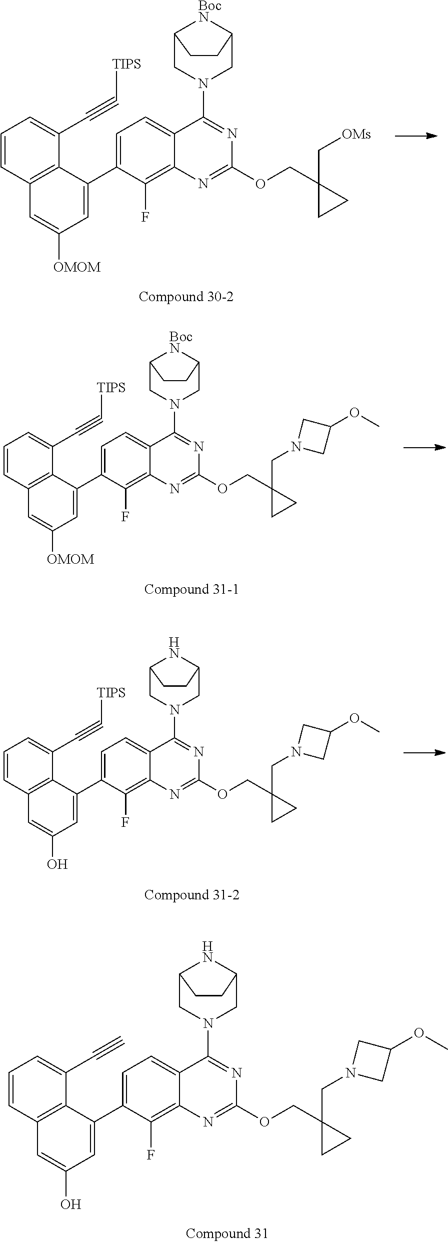

TEA (84.0 mg, 0.85 mmol, 5.0 eq) was added to a solution of 3-methoxyazetidine-HCl (42 mg, 0.34 mmol, 2.0 eq) in DMF (2 mL). The mixture was stirred for 2 hrs at R.T, and then Compound 30-2 (150.0 mg, 0.17 mmol, 1.0 eq) was added. The reaction mixture was stirred for 2 hrs at 80° C., cooled to R.T, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (DCM/MeOH=10:1) to afford Compound 31-1 (32 mg, yield 21.0%) as a yellow oil. LCMS: 894.5 ([M+H]⁺).

A solution of Compound 31-1 (32 mg, 0.035 mmol, 1.0 eq) and HCl/1,4-dioxane (0.4 mL) in MeCN (1.2 mL) was stirred at R.T for 1 h, and then concentrated under reduced pressure to afford a crude product containing Compound 31-2 as a yellow solid which was used directly for the next step without any further purification. LCMS: 750.3 ([M+H]⁺).

A mixture of the crude product containing Compound 31-2, CsF (81 mg, 0.53 mmol, 15.0 eq) and DMF (2 mL) was stirred overnight at 45° C., and then filtered. The filtrate was purified with Prep-HPLC to afford Compound 31 (1.7 mg) as a white solid. LCMS: 594.3 ([M+H]⁺).

### Example 32

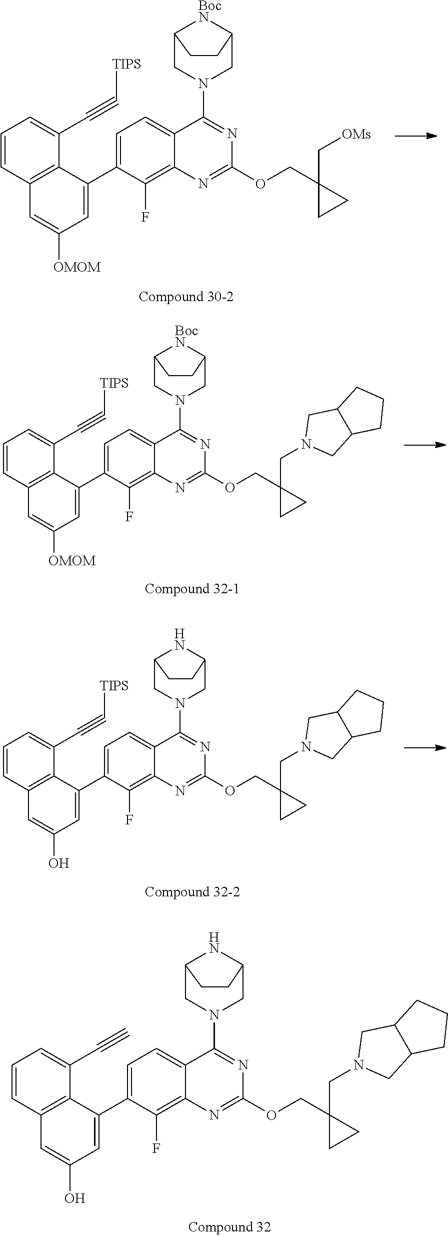

TEA (84.0 mg, 0.85 mmol, 5.0 eq) was added to a solution of octahydrocyclopenta[c]pyrrole-HCl (50 mg, 0.34 mmol, 2.0 eq) in DMF (2 mL). The mixture was stirred for 2 hrs at R.T, and then Compound 30-2 (150.0 mg, 0.17 mmol, 1.0 eq) was added. The reaction mixture was stirred for 2 hrs at 80° C., cooled to R.T, poured into water (5 mL) and extracted with EtOAc (5 mL×2). The organic layers were combined, washed with brine (5 mL), dried with anhydrous Na₂SO₄, and concentrated to obtain a residue which was purified with Pre-TLC (DCM/MeOH=10:1) to afford Compound 32-1 (38 mg, yield 24.0%) as a yellow oil. LCMS: 918.4 ([M+H]⁺).

A solution of Compound 32-1 (38 mg, 0.04 mmol, 1.0 eq) and HCl/dioxane (0.4 mL) in MeCN (1.2 mL) was stirred at R.T for 1 h, and then concentrated under reduced pressure to afford a crude product containing Compound 32-2 as a yellow solid which was used directly for the next step without any further purification. LCMS: 774.4 ([M+H]⁺).

CsF (91 mg, 0.6 mmol, 15.0 eq) was added to a solution of the crude product containing Compound 32-2 in DMF (2 mL) at R.T. The reaction mixture was stirred overnight at 45° C., and then filtered. The filtrate was purified with Prep-HPLC to afford Compound 32 (2.4 mg) as a white solid.

¹H NMR (300 MHz, DMSO-d₆): δ 10.15 (s, 1H), 7.88 (dd, J=7.7, 1.9 Hz, 1H), 7.75 (d, J=8.6 Hz, 1H), 7.50-7.36 (m, 2H), 7.32 (d, J=2.5 Hz, 1H), 7.27-7.15 (m, 1H), 7.00 (d, J=2.5 Hz, 1H), 4.42 (d, J=12.8 Hz, 1H), 4.25 (t, J=7.7 Hz, 3H), 4.05 (s, 2H), 3.62 (dd, J=12.9, 9.1 Hz, 3H), 3.49 (s, 1H), 2.27 (s, 2H), 2.05-1.90 (m, 5H), 1.56 (s, 3H), 1.37 (d, J=20.0 Hz, 4H), 1.24 (s, 3H), 0.65-0.64 (m, 2H), 0.48-0.47 (m, 2H). LCMS: 618.3 ([M+H]⁺).

### Example 33

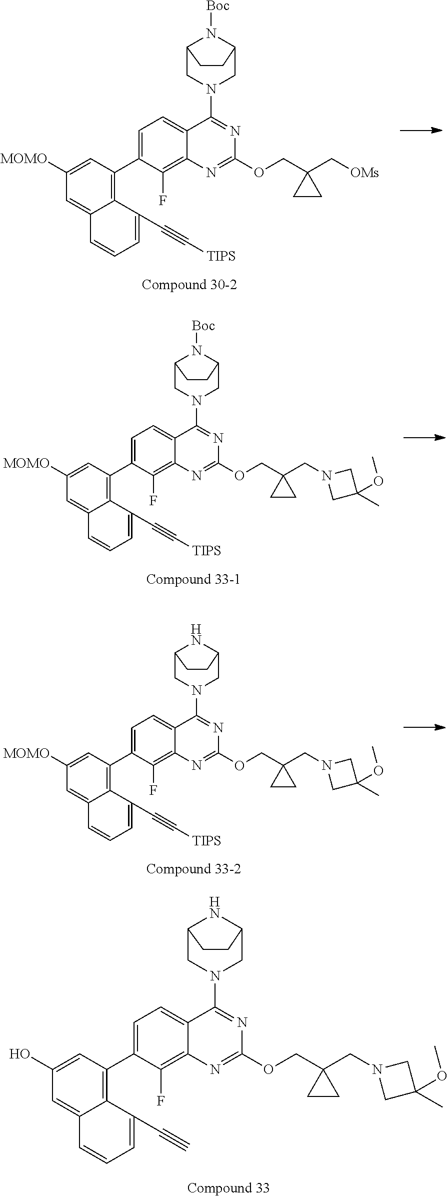

TEA (82.4 mg, 0.81 mmol, 5.0 eq) was added to a solution of 3-methoxy-3-methylazetidine hydrochloride (44.8 mg, 0.33 mmol, 2.0 eq), in DMF (2 mL). The mixture was stirred for 2 hrs at R.T, and then Compound 30-2 (150 mg, 0.16 mmol, 1.0 eq) was added. The reaction mixture was stirred for 2 hrs at 80° C., cooled to R.T, poured into water (5 mL) and then extracted with EtOAc (5 mL×2). The organic layers were combined, washed with water (5 mL) and brine (5 mL) successively, dried with anhydrous Na₂SO₄, and concentrated to obtain a residue which was purified with Pre-TLC (eluting with DCM/MeOH=10:1) to afford Compound 33-1 (45 mg, yield 29.3%) as a yellow oil. LCMS: 908 ([M+H]⁺).

A solution of Compound 33-1 (45 mg, 0.04 mmol, 1.0 eq) and HCl/dioxane (4M, 0.4 mL) in MeCN (2 mL) was stirred for 2 hrs, and then concentrated to afford a crude product containing Compound 33-2 (40 mg) as a yellow solid. LCMS: 764 ([M+H]⁺).

A mixture of the crude product containing Compound 33-2 (45 mg crude, 0.059 mmol, 1.0 eq) and CsF (123 mg, 0.88 mmol, 15.0 eq) in DMF (1 mL) was stirred for 3 hrs at 40° C., and then filtered. The filtrate was purified with Prep-HPLC to afford Compound 33 (6 mg, yield 11.2%) as a white solid.

¹H NMR (400 MHz, CD₃OD-d₄): δ 7.10-7.80 (m, 2H), 7.48-7.47 (m, 1H), 7.40-7.33 (m, 2H), 7.29 (d, J=2.4 Hz, 1H), 7.04 (d, J=2.4 Hz, 1H), 4.64-4.60 (m, 2H), 4.47 (d, J=12.0 Hz, 1H), 4.30-4.27 (m, 1H), 4.22-4.16 (m, 3H), 4.15-4.13 (m, 1H), 3.90-3.84 (m, 2H), 3.21 (s, 3H), 2.92 (s, 1H), 2.18-2.14 (m, 4H), 1.47 (s, 3H), 1.37-1.28 (m, 4H), 0.90-0.85 (m, 4H). LCMS: 608.20 ([M+H]⁺).

### Example 34

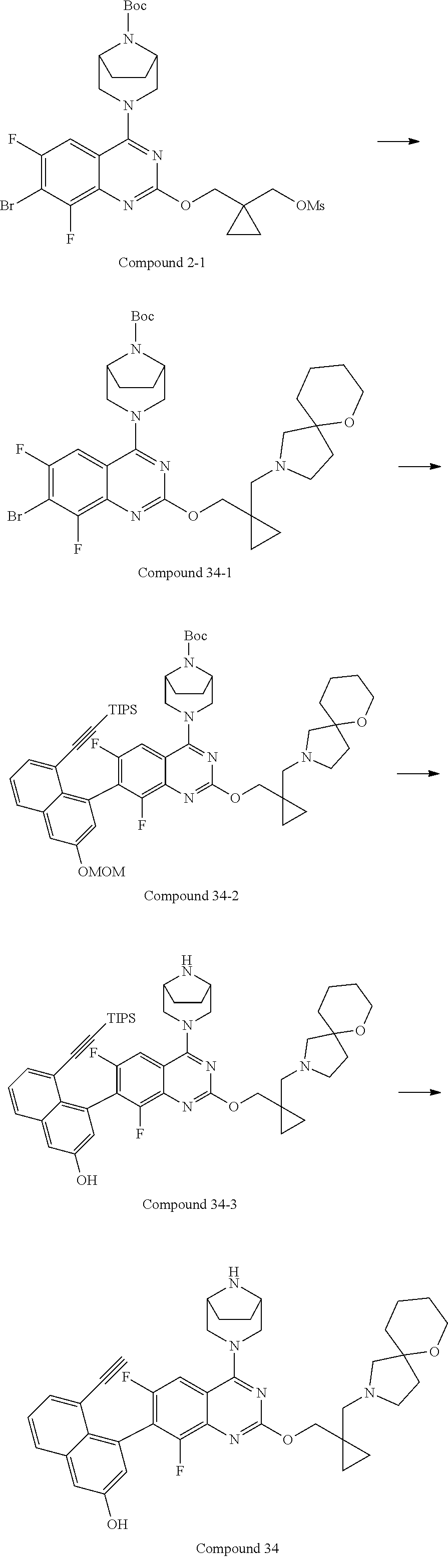

A solution of 6-oxa-2-azaspiro[4.5]decane hydrochloride (127 mg, 714.80 μmol) and triethylamine (0.5 mL) in DMF (5 mL) was stirred at R.T for 2 hrs, and then Compound 2-1 (304 mg, 479.87 μmol) was added. The reaction mixture was purged with nitrogen, stirred at 90° C. for 16 hrs, cooled to R.T, then diluted with EA (50 mL). The resulting mixture was washed with water (2×30 mL). The organic layer was dried over Na₂SO₄ and concentrated under reduced pressure to obtain a residue which was purified with Pre-TLC (EA:HEX=1:1, v/v) to afford Compound 34-1 (154 mg, 226.93 μmol, 47.3% yield). MS m/z: 678 [M+H]⁺.

A mixture of Compound 34-1 (154 mg, 226.93 μmol), INT C1 (163 mg, 329.59 μmol), cataCXium A Pd G₃ (31 mg, 42.56 μmol), Cs₂CO₃ (212 mg, 650.66 μmol), and a mixed solution of toluene (8 mL) and water (2.0 mL) was stirred at 100° C. for 6 hrs under nitrogen atmosphere, and then diluted with EA (50 mL). The resulting mixture was washed with water (2×30 mL). The organic layer were combined, dried over Na₂SO₄ and concentrated under reduced pressure to afford a residue which was purified with Pre-TLC (Hex:EA=1:1, v/v) to afford Compound 34-2 (154 mg, 159.37 μmol, 70.2% yield). MS m/z: 966 [M+H]⁺.

A mixture of Compound 34-2 (154 mg, 159.37 μmol), TFA (1.5 mL) and DCM (5 mL) was stirred at R.T for 1 h, and then diluted with DCM (50 mL). The resulting mixture was washed with saturated aq. NaHCO₃ (2×30 mL), and the organic layer was dried over Na₂SO₄ and concentrated under reduced pressure to afford a crude product containing Compound 34-3 (145 mg). MS m/z: 822 [M+H]⁺.

A mixture of the crude product containing Compound 34-3 (145 mg, 176.37 μmol), CsF (0.49 g, 3.22 mmol) and DMF (5 mL) was stirred at 30° C. for 16 hrs, and then concentrated under reduced pressure to obtain a residue which was purified with Pre-HPLC (YMC-Triart C18 column, 50*250 mm, 7 μm; eluent A: 0.1% TFA in water; eluent B: CH₃CN; gradient: 15% B to 50% B in 40 min at a flow rate of 70 mL/min; 244 nm) to afford Compound 34 (16.8 mg, 25.23 μmol, 14.3% yield). MS m/z: 666 [M+H]⁺.

### Example 35

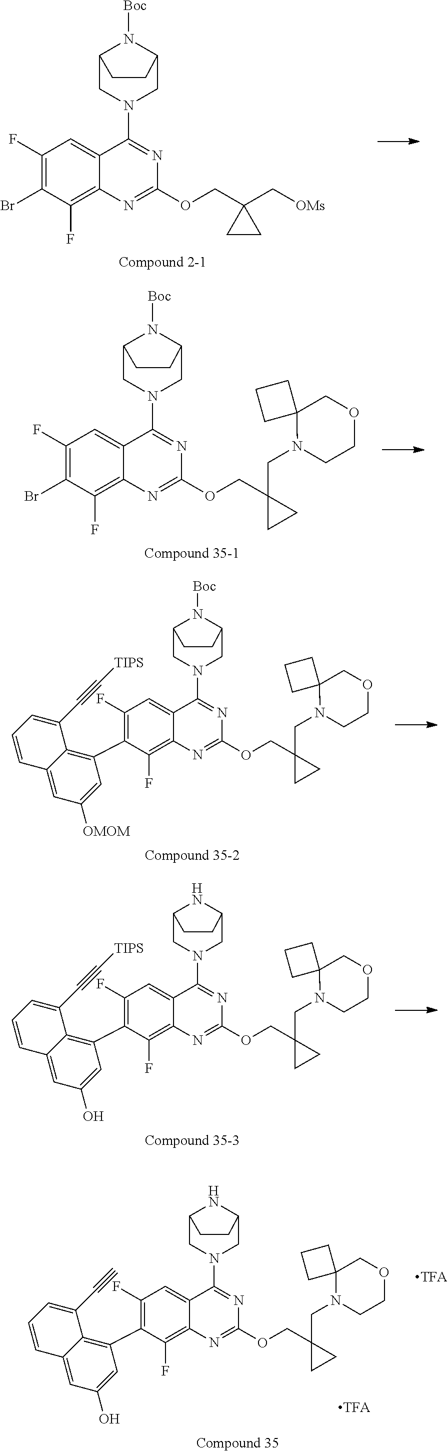

A mixture of 8-oxa-5-azaspiro[3.5]nonane (100 mg, 786.26 μmol), triethylamine (0.5 mL), Compound 2-1 (290 mg, 457.77 μmol) and DMF (5 mL) was purged with nitrogen, stirred at 90° C. for 16 hrs, cooled to R.T, and then diluted with EA (50 mL). The resulting mixture was washed with water (2×30 mL). The organic layer was dried over Na₂SO₄ and concentrated under reduced pressure to obtain a residue which was purified with pre-HPLC (eluting with 0-90% MeCN in water (0.1% TFA)) to afford Compound 35-1 (48 mg, 72.22 μmol, 15.7% yield). MS m/z: 664 [M+H]⁺.

A mixture of Compound 35-1 (48 mg, 72.22 μmol), INT C1 (56 mg, 113.23 μmol), cataCXium A Pd G₃ (12 mg, 16.47 μmol), Cs₂CO₃ (70 mg, 214.84 μmol), and a mixed solution of toluene (4 mL) and water (1.0 mL) was stirred at 100° C. for 16 hrs under nitrogen atmosphere, and then diluted with EA (50 mL). The resulting mixture was washed with water (2×30 mL). The organic layer was dried over Na₂SO₄ and concentrated under reduced pressure to obtain a residue which was purified with Pre-TLC (MeOH:DCM=1:15, v/v) to afford Compound 35-2 (57 mg, 59.85 μmol, 82.8% yield). MS m/z: 952 [M+H]⁺.

A mixture of Compound 35-2 (57 mg, 59.85 μmol), HCl (4 M in 1,4-dioxane, 1.5 mL) and CH₃CN (5 mL) was stirred at R.T for 1 h, and then diluted with EA (50 mL). The resulting mixture was washed with saturated aq. NaHCO₃ (2×30 mL). The organic layer was dried over Na₂SO₄ and concentrated under reduced pressure to afford a crude product containing Compound 35-3 (58 mg, 71.77 μmol, 119.9% yield). MS m/z: 808 [M+H]⁺.

A mixture of the crude product containing Compound 35-3 (58 mg, 71.77 μmol), CsF (242 mg, 1.59 mmol) and DMF (4 mL) was stirred at 30° C. for 16 hrs, and then concentrated under reduced pressure to obtain a residue which was purified with Pre-HPLC (Agela Durashell C18 column, 30 mm*250 mm, 10 um; eluent A: 0.1% TFA in water, eluent B: CH₃CN; gradient: 10% B to 40% B in 35 min at a flow rate of 40 mL/min; 235 nm) to afford a TFA salt of Compound 35 (20.6 mg, 23.41 μmol, 32.6% yield, TFA salt). MS m/z: 652 [M+H]⁺.

### Example 36

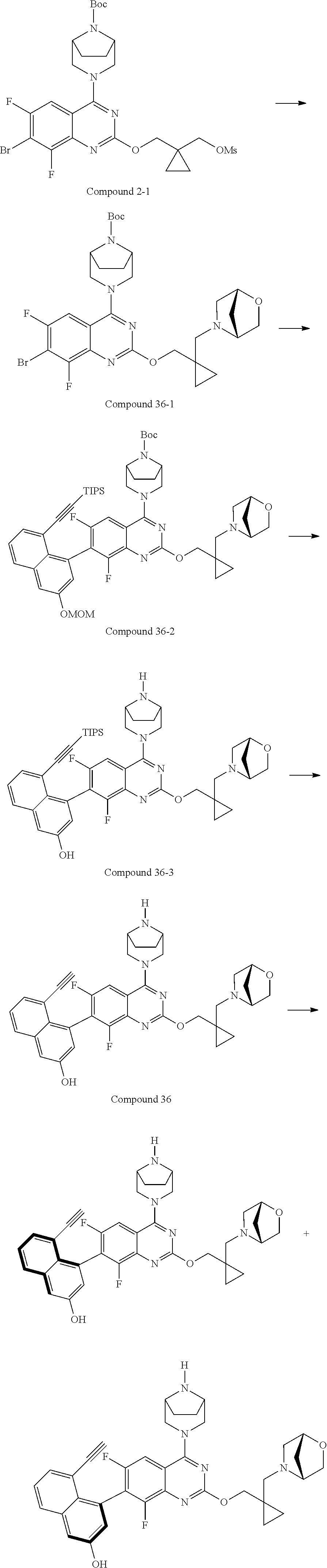

A mixture of (1R,4R)-2-oxa-5-azabicyclo[2.2.1]heptane (95 mg, 958.33 μmol), triethylamine (0.5 mL), Compound 2-1 (306 mg, 483.03 μmol) and DMF (5 mL) was purged with nitrogen, stirred at 90° C. for 18 hrs, cooled to R.T and then diluted with EA (50 mL). The resulting mixture was washed with water (2×30 mL). The organic layer was dried over Na₂SO₄ and concentrated under reduced pressure to obtain a residue which was purified with Pre-TLC (EA:Hex=2:3, v/v) to afford Compound 36-1 (157 mg, 246.65 μmol, 51.06% yield). MS m/z: 636.19 [M+H]⁺.

A mixture of Compound 36-1 (157 mg, 246.65 μmol), INT C1 (164 mg, 331.6 μmol), cataCXium A Pd G₃ (28 mg, 38.45 μmol), Cs₂CO₃ (270 mg, 828.68 μmol), toluene (4 mL) and water (1.0 mL) was stirred at 100° C. for 16 hrs under nitrogen atmosphere, and then diluted with EA (100 mL). The resulting mixture was washed with water (2×40 mL). The organic layer was dried over Na₂SO₄ and concentrated under reduced pressure to obtain a residue which was purified with Pre-TLC (MeOH:DCM=1:15, v/v) to afford Compound 36-2 (173 mg, 187.19 μmol, 75.89% yield). MS m/z: 924.22 [M+H]⁺.

A mixture of Compound 36-2 (173 mg, 187.19 μmol) and HCl (4 M in 1,4-dioxane, 2 mL) and CH₃CN (6 mL) was stirred at R.T for 2 hrs, and then diluted with EA (50 mL). The resulting mixture was washed with saturated aq. NaHCO₃ (2×20 mL). The organic layer was dried over Na₂SO₄ and concentrated under reduced pressure to afford a crude product containing Compound 36-3 (153 mg). MS m/z: 780.05 [M+H]⁺.

A mixture of Compound 36-3 (153 mg, 196.15 μmol), CsF (711 mg, 4.68 mmol) and DMF (5 mL) was stirred at R.T for 16 hrs, and then concentrated under reduced pressure to obtain a residue which was purified with Pre-HPLC (Daisogel-C18 column, 50*250 mm, 10 μm; eluent A: 0.1% TFA in water; eluent B: CH₃CN; gradient: 20% B to 35% B in 30 min at a flow rate of 60 mL/min; 254 nm) to afford Compound 36 (83.9 mg, 98.50 μmol, 50.2% yield). MS m/z: 624 [M+H]⁺.

The enantio separation of the Compound 36 was performed by chiral-HPLC with the following condition: CHIRAL ART Cellulose-SA column (2 cm×25 cm, 5 um) on Prep-HPLC-Gilson; Mobile phase: Hex (0.1% IPA.M)/EtOH (50:50); Flow rate: 20 mL/min. This resulted in a first eluting stereoisomer (Compound 36A, 22.3 mg, Retention Time 3.639 min) and a second eluting stereoisomer (Compound 36B, 17.9 mg, Retention Time 6.608 min).

### Example 37

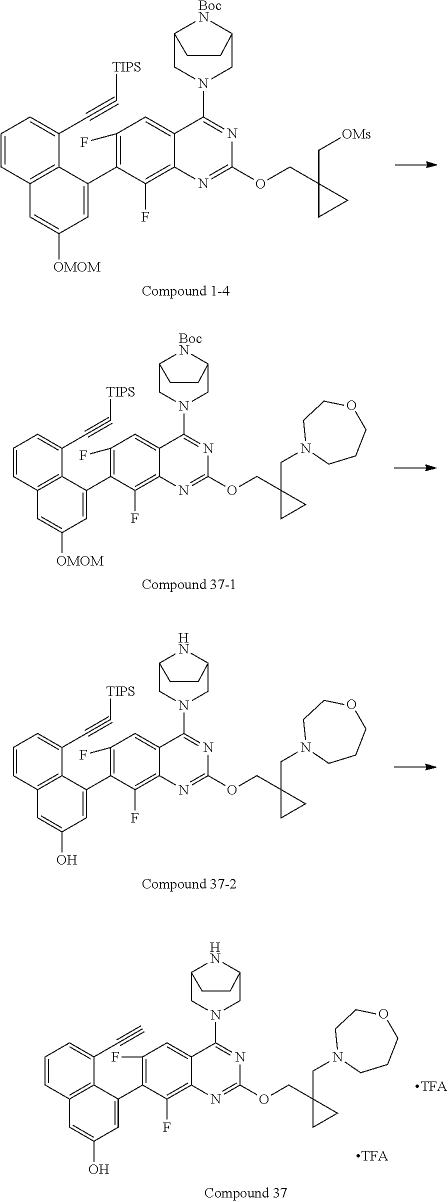

A mixture of Compound 1-4 (150.0 mg, 0.16 mmol, 1.0 eq), 1,4-oxazepane (19.7 mg, 0.195 mmol, 1.2 eq) and DMF (1.5 mL) was stirred at 85° C. for 3 hrs, cooled to R.T, poured into water (5 mL) and then extracted with EtOAc (5 mL×2). The organic layers were combined, washed with brine (5 mL), dried with anhydrous Na₂SO₄, and concentrated to obtain a residue which was purified with Pre-TLC (V_{DCM}/V_{MeOH}=30:1) to afford Compound 37-1 (53 mg, 0.057 mmol, 35.1% yield) as a yellow oil. LCMS m/z: 926.4 [M+H]⁺.

A solution of Compound 37-1 (53 mg, 0.057 mmol, 1.0 eq) and HCl/dioxane (0.54 mL) in MeCN (2.4 mL) was stirred at R.T for 1 h, and then concentrated under reduced pressure to afford a crude product containing Compound 37-2 as a yellow solid which was used directly for the next step without any further purification. LCMS m/z: 782.4 [M+H]⁺.

A mixture of the crude product containing Compound 37-2, CsF (130.4 mg, 0.858 mmol, 15.0 eq) and DMF (1.9 mL) was stirred at 40° C. for 2.5 hrs, and then filtered. The filtrate was purified with Prep-HPLC to afford the TFA salt of Compound 37 (4.5 mg) as a white solid. LCMS m/z: 626.3 [M+H]⁺.

### Example 38

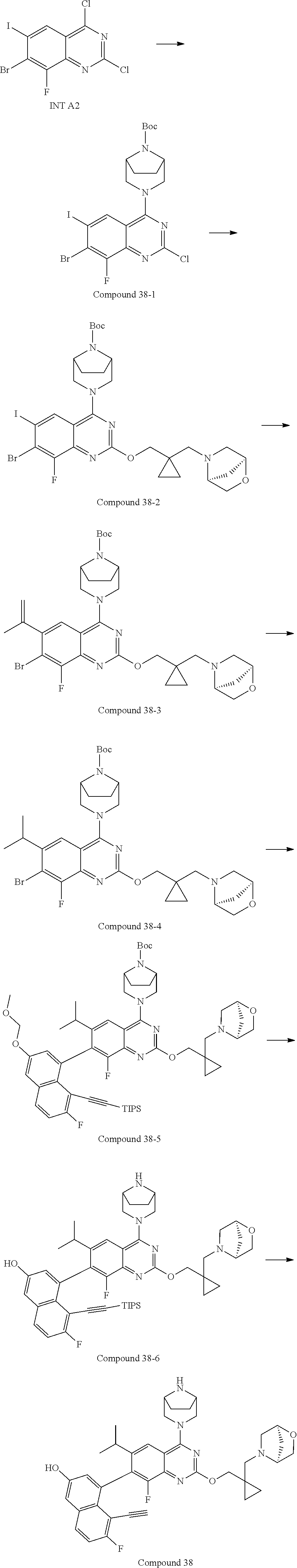

A mixture of INT A2 (18.24 g, 43.24 mmol), DIEA (10.61 g, 82.09 mmol) and tert-butyl (1R,5S)-3,8-diazabicyclo[3.2.1]octane-8-carboxylate (8.90 g, 41.92 mmol) and DCM (150 mL) was stirred at room temperature for 2 hrs. The resulting mixture was diluted with water (200 mL), and then extracted with DCM (200 mL). The organic layer was dried over Na₂SO₄ and concentrated in vacuum to obtain a residue. The residue was dispersed in Hex/EA (20:1, v/v, 240 ml) and then filtered. The filter cake was collected and dried to afford Compound 38-1 (20.48 g, 34.27 mmol). MS m/z: 597 [M+H]⁺.

A mixture of Compound 38-1 (8.00 g, 13.38 mmol), INT B1 (5.00 g, 27.28 mmol), potassium fluoride (2.37 g, 40.79 mmol), and methyl sulfoxide (120 mL) was stirred at 120° C. for 16 hrs, cooled to room temperature and then water (100 mL) was added. The resulting mixture was extracted with EA (2×200 mL). The combined organic layers were dried over Na₂SO₄ and concentrated in vacuum to obtain a residue which was purified with silica gel column chromatography (eluted with 0-75% EA in Hex) to afford Compound 38-2 (5.31 g, 7.13 mmol, 53.2% yield). MS m/z: 744 [M+H]⁺.

To a solution of Compound 38-2 (1372 mg, 1.84 mmol), 4,4,5,5-tetramethyl-2-(prop-1-en-2-yl)-1,3,2-dioxaborolane (332 mg, 1.98 mmol) in 1,4-dioxane (10 mL) were added K₃PO₄ (813 mg, 3.83 mmol) and Pd(dppf)Cl₂·CH₂Cl₂ (176 mg, 0.22 mmol). The reaction mixture was stirred at 80° C. for 3.5 hrs under nitrogen atmosphere, and then filtered. The filtrate was concentrated under vacuum to obtain a residue which was purified with Pre-HPLC (chromatographic column: C18 column; mobile phase A: 0.1% TFA in water; mobile phase B: CH₃CN; gradient: 25% B to 60% B in 35 min; flow rate: 80 mL/min) to afford Compound 38-3 (571 mg, 0.87 mmol). MS m/z: 658/660 [M+H]⁺.

To a solution of Compound 38-3 (571 mg, 0.87 mmol) in EA (30 mL) was added PtO₂ (108 mg). The reaction mixture was stirred at RT for 3 hrs under an atmosphere of H₂, and then filtered. The filtrate was concentrated under vacuum to obtain a residue which was purified with Pre-HPLC (chromatographic column: C18 column; mobile phase A: 0.1% TFA in water; mobile phase B: CH₃CN; gradient: 25% B to 60% B in 35 min; flow rate: 80 mL/min) to afford Compound 38-4 (318 mg, 0.48 mmol). MS m/z: 660/662 [M+H]⁺.

To a solution of Compound 38-4 (318 mg, 0.48 mmol), INT C2 (349 mg, 0.68 mmol) in toluene (10 mL) and water (2.5 mL) were added Cs₂CO₃ (369 mg, 1.13 mmol) and cataCXium A Pd G3 (76 mg, 0.10 mmol). The reaction mixture was stirred overnight at 100° C. under an atmosphere of nitrogen, diluted with water (20 mL) and extracted with EA (2×20 mL). The organic layers were combined, dried over Na₂SO₄ and concentrated under vacuum to obtain a residue. The residue was purified with Pre-HPLC (chromatographic column: C18 column; mobile phase A: 0.1% TFA in water; mobile phase B: CH₃CN; gradient: 25% B to 60% B in 35 min and 60% B to 100% B in 15 min; flow rate: 50 mL/min) to afford Compound 38-5 (286 mg, 0.30 mmol). MS m/z: 966 [M+H]⁺.

A solution of Compound 38-5 (286 mg, 0.30 mmol) and HCl (1.5 mL, 4 M in dioxane) in MeCN (4.5 mL) was stirred at RT for 1 h, and then concentrated in vacuum to obtain a residue. The residue was diluted with sat. NaHCO₃ (20 mL) and extracted with EA (2×20 mL). The organic layers were combined, dried over Na₂SO₄ and concentrated under vacuum to afford Compound 38-6 (191 mg, 0.23 mmol). MS m/z: 822 [M+H]⁺.

To a solution of Compound 38-6 (191 mg, 0.23 mmol) in DMF (5 ml) was added CsF (0.83 g, 5.46 mmol). The reaction mixture was stirred overnight at 40° C., and then filtered. The filtrate was concentrated under vacuum to obtain a residue. The residue was purified with Prep-HPLC (chromatographic column: C18 column; mobile phase A: 0.1% TFA in water; mobile phase B: CH₃CN; gradient: 15% B to 55% B in 60 min; flow rate: 200 mL/min; ultraviolet wavelength: 240 nm) to afford a TFA of Compound 38 (101.4 mg, 0.11 mmol). MS m/z: 666 [M+H]⁺.

### Example 39

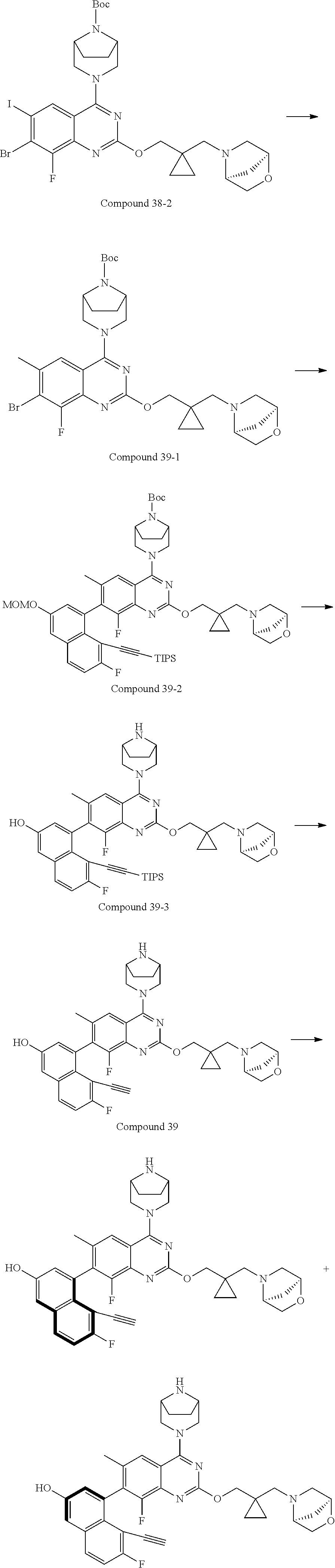

A mixture of Compound 38-2 (504 mg, 677.02 μmol), potassium carbonate (200 mg, 1.44 mmol), Pd(dppf)Cl₂ (69 mg, 84.91 μmol), 2,4,6-trimethyl-1,3,5,2,4,6-trioxatriborinane (268 mg, 1.06 mmol), 1,4-dioxane (8 mL) and water (2 mL) was stirred at 90° C. for 16 hrs under an atmosphere of nitrogen, cooled to room temperature, diluted with EA (50 mL) and washed with water (30 mL). The organic layer was dried over Na₂SO₄ and concentrated under vacuum to obtain a residue which was purified with Pre-TLC (DCM:MeOH=1:20, v/v) to afford Compound 39-1 (206 mg, 325.65 μmol, 48.1% yield). MS m/z: 632 [M+H]⁺.

A mixture of Compound 39-1 (206 mg, 325.65 μmol), INT C2 (252 mg, 491.67 μmol), cataCXium A Pd G3 (36 mg, 49.43 μmol), Cs₂CO₃ (337 mg, 1.03 mmol), toluene (8 mL) and water (2 mL) was stirred at 100° C. for 16 hrs under an atmosphere of nitrogen, diluted with EA (50 mL) and washed with water (2×30 mL). The organic layer was dried over Na₂SO₄ and concentrated under vacuum to obtain a residue which was purified with Pre-TLC (MeOH:DCM=1:20, v/v) to afford Compound 39-2 (258 mg, 274.98 μmol, 84.4% yield). MS m/z: 938 [M+H]⁺.

To a solution of Compound 39-2 (258 mg, 274.98 μmol) in CH₃CN (6 mL) was added HCl (4 M in 1,4-dioxane, 2 mL). The reaction mixture was stirred at room temperature for 1 h, and then concentrated under reduced pressure to afford Compound 39-3 (224 mg, 282.09 μmol, 102.5% yield). MS m/z: 794 [M+H]⁺.

To a solution of Compound 39-3 (224 mg, 282.09 μmol) in DMF (3 mL) was added CsF (0.60 g, 3.94 mmol). The reaction mixture was stirred at 40° C. for 16 hrs, and then concentrated under reduced pressure to obtain a residue which was purified with Pre-HPLC (chromatographic column: YMC-Triart C18-S12 nm, 50*250 mm, 7 μm; mobile phase A: 0.1% TFA in water; mobile phase B: CH₃CN; gradient: 15% B to 40% B in 30 min; flow rate of 70 mL/min; ultraviolet wavelength: 240 nm) to afford Compound 39 (213 mg, 334.00 μmol, 118.4% yield). MS m/z: 638 [M+H]⁺.

Compound 39 (213 mg, 334.00 μmol) was separated by Prep-HPLC-Gilson with the following conditions: column, CHIRAL ART Cellulose-SA column (2 cm×25 cm, 5 um); mobile phase, Hex (0.1% IPA.M)/EtOH (50:50); Flowing rate: 20 ml/min to afford Compound 39A (32.2 mg, retention time 4.217 min) and Compound 39B (28.4 mg, retention time 5.886 min) respectively.

### Example 40

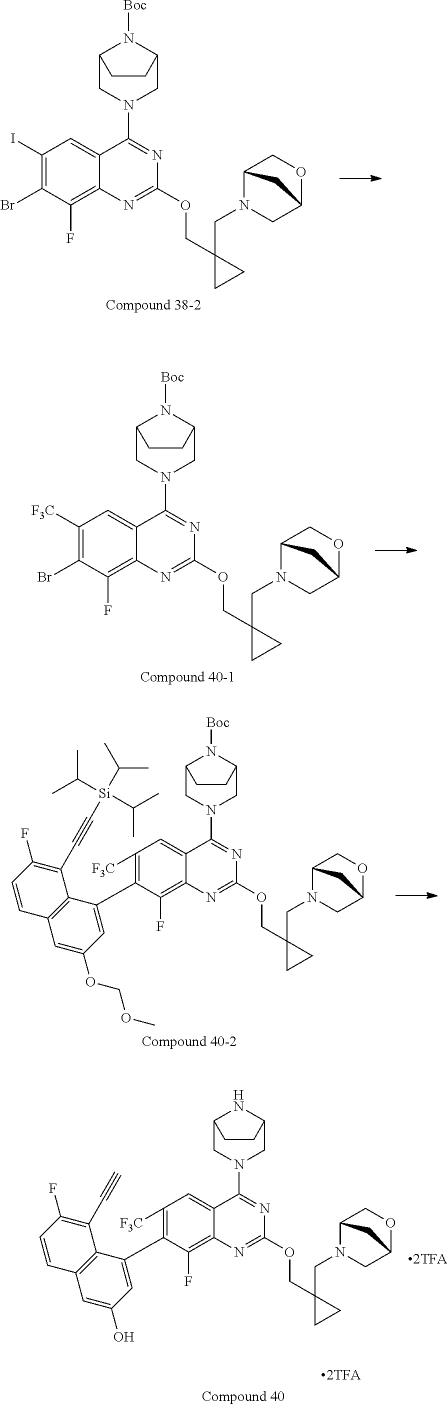

To a solution of Compound 38-2 (1022 mg, 1.37 mmol), diphenyl-(trifluoromethyl)-sulfonium trifluoromethanesulfonate (890 mg, 2.20 mmol) in NMP (10 mL) was added copper powder (263 mg, 4.14 mmol). The reaction mixture was stirred overnight at 60° C. under an atmosphere of nitrogen, and then filtered. The filtrate was concentrated under vacuum to obtain a residue. The residue was purified by Pre-HPLC (chromatographic column: C18 column; mobile phase A: 0.1% TFA in water; mobile phase B: CH₃CN; gradient: 30% B to 60% B in 30 min; flow rate: 80 mL/min; ultraviolet wavelength: 254 nm) to afford Compound 40-1 (466 mg, 0.679 mmol). MS m/z: 686/688 [M+H]⁺.

To a solution of Compound 40-1 (213 mg, 0.310 mmol), INT C2 (252 mg, 0.492 mmol) in toluene (5 mL) and water (1 mL) were added Cs₂CO₃ (270 mg, 0.829 mmol) and cataCXium A Pd G3 (24 mg, 0.330 mmol). The reaction mixture was stirred overnight at 100° C. under an atmosphere of nitrogen, diluted with water (20 mL) and extracted with EA (2×20 mL). The organic layers were combined, dried over Na₂SO₄ and concentrated under vacuum to obtain a residue which was purified with Pre-TLC (Hex:EA=1:15, v/v) to afford Compound 40-2: (88 mg, 0.308 mmol). MS m/z: 992 [M+H]⁺.

A solution of Compound 40-2 (88 mg, 0.308 mmol), HCl (1 mL, 1 M in dioxane) in MeCN (3 mL) was stirred at RT for 1 h, and then concentrated in vacuum to obtain a residue. The residue was dispersed in sat. NaHCO₃ (10 mL) and extracted with EA (2×10 mL). The organic layers were combined, dried over Na₂SO₄ and concentrated under vacuum to obtain a residue. CsF (310 mg, 2.04 mmol) was added to a mixture of the residue and DMF (3 mL), and the reaction mixture was stirred overnight at 40° C. under an atmosphere of nitrogen. The reaction mixture was purified with Prep-HPLC (chromatographic column: C18 column; mobile phase A: 0.1% TFA in water; mobile phase B: CH₃CN; gradient: 10% B to 40% B in 40 min; flow rate: 70 mL/min; ultraviolet wavelength: 240 nm) to afford a TFA of Compound 40 (40.1 mg, 0.436 mmol). MS m/z: 692 [M+H]⁺.

### Example 41

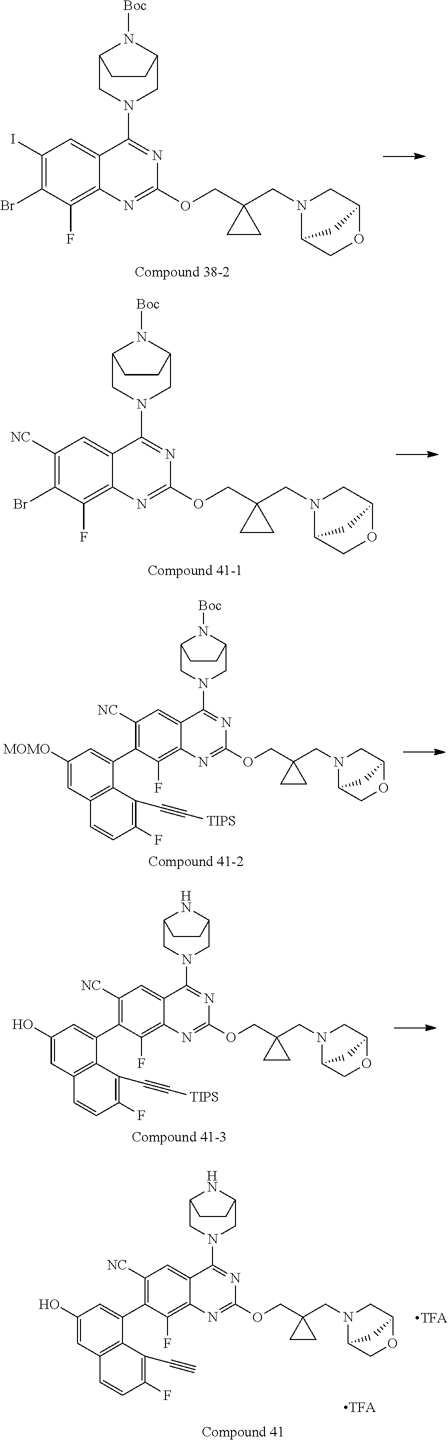

To a mixture of Compound 38-2 (500 mg, 0.67 mmol), zinc cyanide (125 mg, 1.06 mmol) in DMF (15 mL) was added Pd(PPh₃)₄ (104 mg, 0.090 mmol). The reaction mixture was stirred overnight at 100° C. under an atmosphere of nitrogen, and then filtered. The filtrate concentrated under vacuum to obtain a residue which purified with Pre-TLC to afford Compound 41-1 (364 mg, 0.57 mmol). MS m/z: 643/645 [M+H]⁺.

Cs₂CO₃ (405 mg, 1.24 mmol) and cataCXium A Pd G3 (77 mg, 0.11 mmol) were added to a solution of Compound 41-1 (364 mg, 0.57 mmol) and INT C2 (377 mg, 0.74 mmol) dissolved in a mixed solvent of toluene (10 mL) and water (2.5 mL). The reaction mixture was stirred overnight at 100° C. under an atmosphere of nitrogen, diluted with water (20 mL) and extracted with EA (2×20 mL). The organic layers were combined, dried over Na₂SO₄ and concentrated under vacuum. The residue was purified with Pre-TLC to afford Compound 41-2 (363 mg, 0.54 mmol). MS m/z: 949 [M+H]⁺.

A solution of Compound 41-2 (363 mg, 0.54 mmol), HCl (1.5 mL, 4 M in dioxane) in MeCN (4.5 mL) was stirred at RT for 1 h, and then concentrated in vacuum to obtain a residue. The residue was dispersed in sat. NaHCO₃ (20 mL) and extracted with EA (2×20 mL). The organic layers were combined, dried over Na₂SO₄ and concentrated under vacuum to afford Compound 41-3 (299 mg, 0.37 mmol). MS m/z: 805 [M+H]⁺.

To a solution of Compound 41-3 (299 mg, 0.37 mmol) in DMF (5 ml) was added CsF (0.98 g, 6.45 mmol). The reaction mixture was stirred overnight at 40° C., and then filtered. The filtrate was concentrated under vacuum to obtain a residue which was purified with Prep-HPLC (chromatographic column: C18 column; mobile phase A: 0.1% TFA in water; mobile phase B: CH₃CN; gradient: 15% B to 55% B in 60 min; flow rate: 200 mL/min; ultraviolet wavelength: 240 nm) to afford a TFA of Compound 41 (198.7 mg, 0.23 mmol). MS m/z: 649 [M+H]⁺.

### Example 42

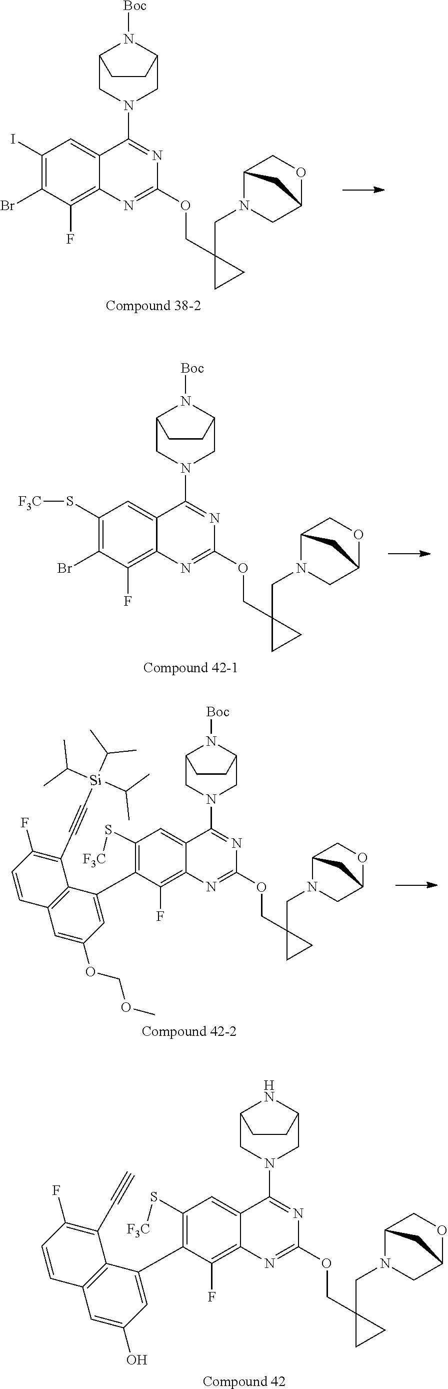

To a solution of Compound 38-2 (510 mg, 0.685 mmol), (Trifluoromethylthio) silver (I) (416 mg, 1.99 mmol) in DMF (7.5 mL) was added CuI (125 mg, 0.656 mmol). The reaction mixture was stirred overnight at 95° C. under an atmosphere of nitrogen, and then filtrated. The filtrate concentrated under vacuum to obtain a residue which was purified with Pre-TLC (EA) to afford Compound 42-1 (164 mg, 0.228 mmol). MS m/z: 718/720 [M+H]⁺.

To a solution of Compound 42-1 (164 mg, 0.228 mmol) and INT C2 (156 mg, 0.304 mmol) dissolved in a mixed solvent of toluene (5 mL) and water (1 mL) were added Cs₂CO₃ (206 mg, 0.632 mmol) and cataCXium A Pd G3 (17 mg, 0.023 mmol). The reaction mixture was stirred overnight at 100° C. under an atmosphere of nitrogen, diluted with water (20 mL) and extracted with EA (2×20 mL). The organic layers were combined, dried over Na₂SO₄ and concentrated under vacuum. The residue was purified by Pre-TLC (Hex:EA=1:15, v/v) to give the desired product Compound 42-2 (127 mg, 0.0955 mmol). MS m/z: 1024 [M+H]⁺.

A solution of Compound 42-1 (127 mg, 0.0955 mmol), HCl (1 mL, 1 M in dioxane) in MeCN (3 mL) was stirred at RT for 1 h, and then concentrated in vacuum to obtain a mixture. The mixture was dispersed in sat. NaHCO₃ (10 mL) and extracted with EA (2×10 mL). The organic layers were combined, dried over Na₂SO₄ and concentrated under vacuum to obtain a residue. A mixture of the residue, DMF (3 mL) and CsF (410 mg, 2.70 mmol) was stirred overnight at 40° C. under an atmosphere of nitrogen. The resulting mixture was purified with Prep-HPLC (chromatographic column: C18 column; mobile phase A: 0.1% NH₃*H₂O in water; mobile phase B: CH₃CN; gradient: 20% B to 55% B in 30 min; flow rate: 70 mL/min; ultraviolet wavelength: 240 nm) to afford Compound 42 (25.7 mg, 0.036 mmol). MS m/z: 724 [M+H]⁺.

### Example 43

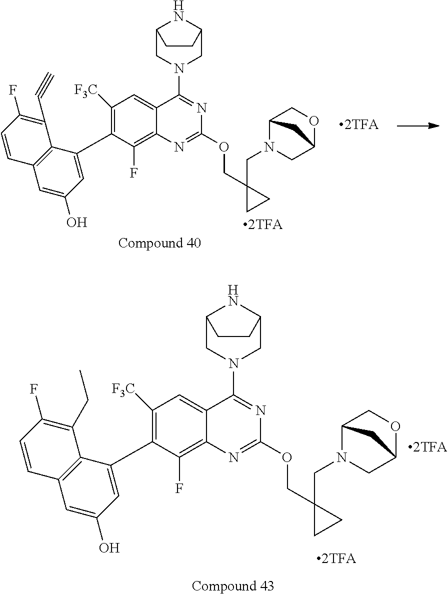 [SMILES: C#Cc1c(F)ccc2cc(O)cc(-c3c(C(F)(F)F)cc4c(N5CC6CCC(C5)N6)nc(OCC5(CN6C[C@@H]7C[C@H]6CO7)CC5)nc4c3F)c12 ; "Compound 40" ; CCc1c(F)ccc2cc(O)cc(-c3c(C(F)(F)F)cc4c(N5CC6CCC(C5)N6)nc(OCC5(CN6C[C@@H]7C[C@H]6CO7)CC5)nc4c3F)c12 ; "Compound 43"]

To a solution of the 2 TFA salt of Compound 40 (17.7 mg, 0.019 mmol) in MeOH (5 mL) was added Pd(OH)₂/C (16.4 mg, 20% wt). The reaction mixture was stirred at RT for 3 hrs under an atmosphere of hydrogen, and then filtered. The filtrate was concentrated under vacuum to obtain a residue which was lyophilized to afford the 2 TFA of Compound 43 (15.4 mg, 0.017 mmol). MS m/z: 696 [M+H]⁺.

The following compounds could be synthesized with reference to the synthesis procedures of Example 1:

| Compound | Structure |
|---|---|
| 44 | 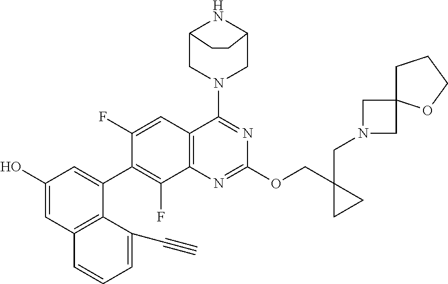 [SMILES: C#Cc1cccc2cc(O)cc(-c3c(F)cc4c(N5CC6CCC(C5)N6)nc(OCC5(CN6CC7(CCCO7)C6)CC5)nc4c3F)c12] |
| 45 | 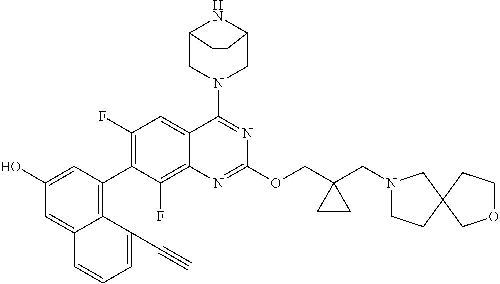 [SMILES: C#Cc1cccc2cc(O)cc(-c3c(F)cc4c(N5CC6CCC(C5)N6)nc(OCC5(CN6CCC7(CCOC7)C6)CC5)nc4c3F)c12] |
| 46 | 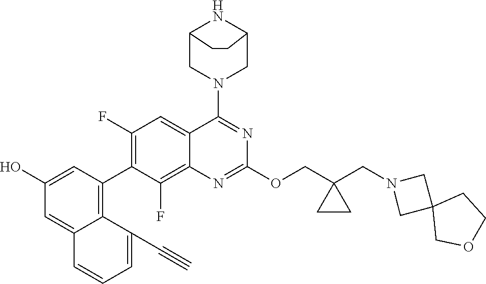 [SMILES: C#Cc1cccc2cc(O)cc(-c3c(F)cc4c(N5CC6CCC(C5)N6)nc(OCC5(CN6CC7(CCOC7)C6)CC5)nc4c3F)c12] |
| 47 | 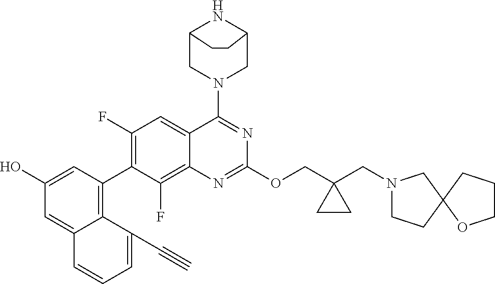 [SMILES: C#Cc1cccc2cc(O)cc(-c3c(F)cc4c(N5CC6CCC(C5)N6)nc(OCC5(CN6CCC7(CCCO7)C6)CC5)nc4c3F)c12] |
| 48 | 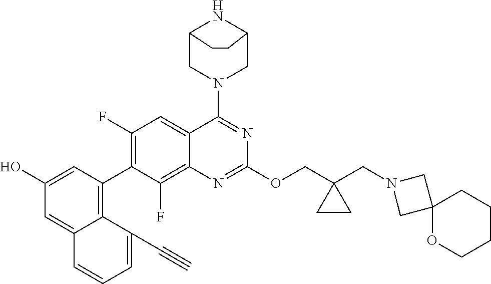 [SMILES: C#Cc1cccc2cc(O)cc(-c3c(F)cc4c(N5CC6CCC(C5)N6)nc(OCC5(CN6CC7(CCCCO7)C6)CC5)nc4c3F)c12] |
| 49 | 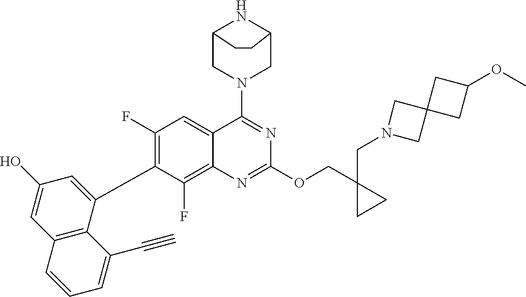 [SMILES: C#Cc1cccc2cc(O)cc(-c3c(F)cc4c(N5CC6CCC(C5)N6)nc(OCC5(CN6CC7(CC(OC)C7)C6)CC5)nc4c3F)c12] |
| 50 | 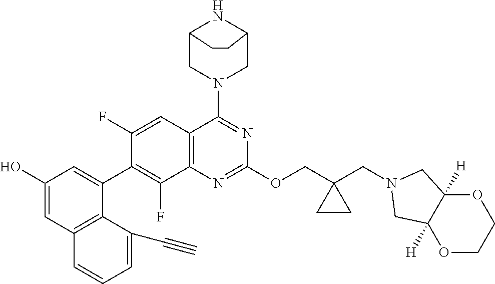 [SMILES: C#Cc1cccc2cc(O)cc(-c3c(F)cc4c(N5CC6CCC(C5)N6)nc(OCC5(CN6C[C@@H]7OCCO[C@@H]7C6)CC5)nc4c3F)c12] |
| 51 | 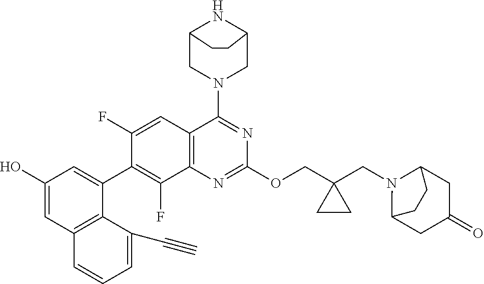 [SMILES: C#Cc1cccc2cc(O)cc(-c3c(F)cc4c(N5CC6CCC(C5)N6)nc(OCC5(CN6C7CCC6CC(=O)C7)CC5)nc4c3F)c12] |
| 52 | 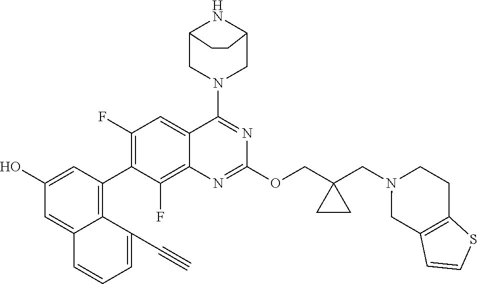 [SMILES: C#Cc1cccc2cc(O)cc(-c3c(F)cc4c(N5CC6CCC(C5)N6)nc(OCC5(CN6CCc7sccc7C6)CC5)nc4c3F)c12] |
| 53 | 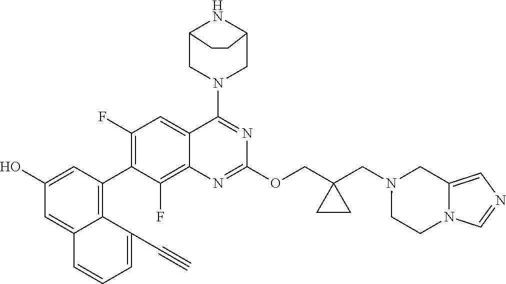 [SMILES: C#Cc1cccc2cc(O)cc(-c3c(F)cc4c(N5CC6CCC(C5)N6)nc(OCC5(CN6CCn7cncc7C6)CC5)nc4c3F)c12] |
| 54 | 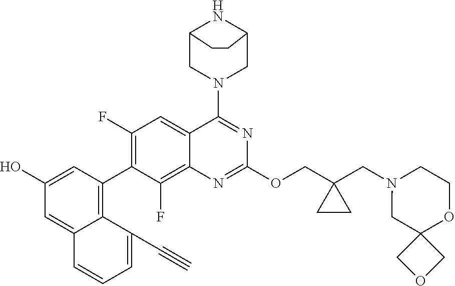 [SMILES: C#Cc1cccc2cc(O)cc(-c3c(F)cc4c(N5CC6CCC(C5)N6)nc(OCC5(CN6CCOC7(COC7)C6)CC5)nc4c3F)c12] |
| 55 | 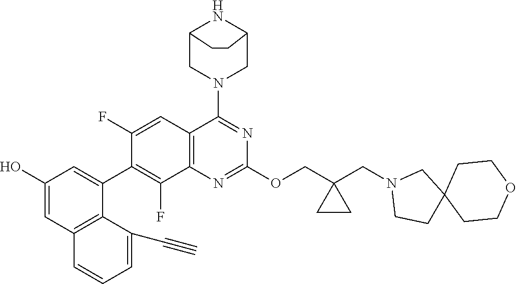 [SMILES: C#Cc1cccc2cc(O)cc(-c3c(F)cc4c(N5CC6CCC(C5)N6)nc(OCC5(CN6CCC7(CCOCC7)C6)CC5)nc4c3F)c12] |
| 56 | 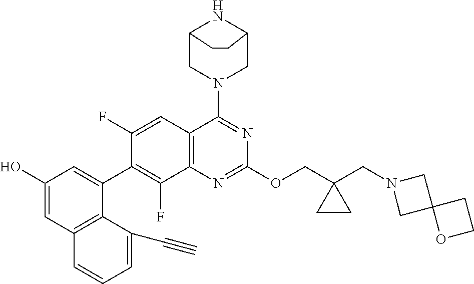 [SMILES: C#Cc1cccc2cc(O)cc(-c3c(F)cc4c(N5CC6CCC(C5)N6)nc(OCC5(CN6CC7(CCO7)C6)CC5)nc4c3F)c12] |
| 57 | 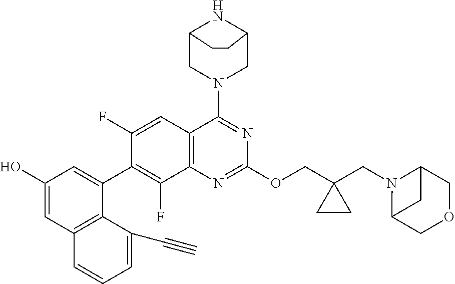 [SMILES: C#Cc1cccc2cc(O)cc(-c3c(F)cc4c(N5CC6CCC(C5)N6)nc(OCC5(CN6C7COCC6C7)CC5)nc4c3F)c12] |
| 58 | 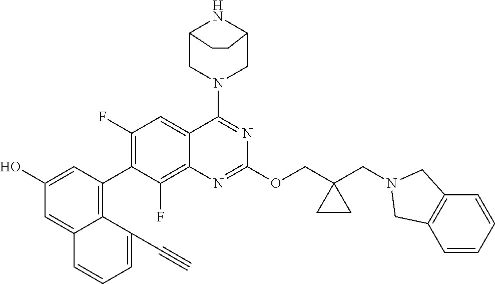 [SMILES: C#Cc1cccc2cc(O)cc(-c3c(F)cc4c(N5CC6CCC(C5)N6)nc(OCC5(CN6Cc7ccccc7C6)CC5)nc4c3F)c12] |

The following compounds could be synthesized with reference to the synthesis procedures of Example 4 and Example 43:

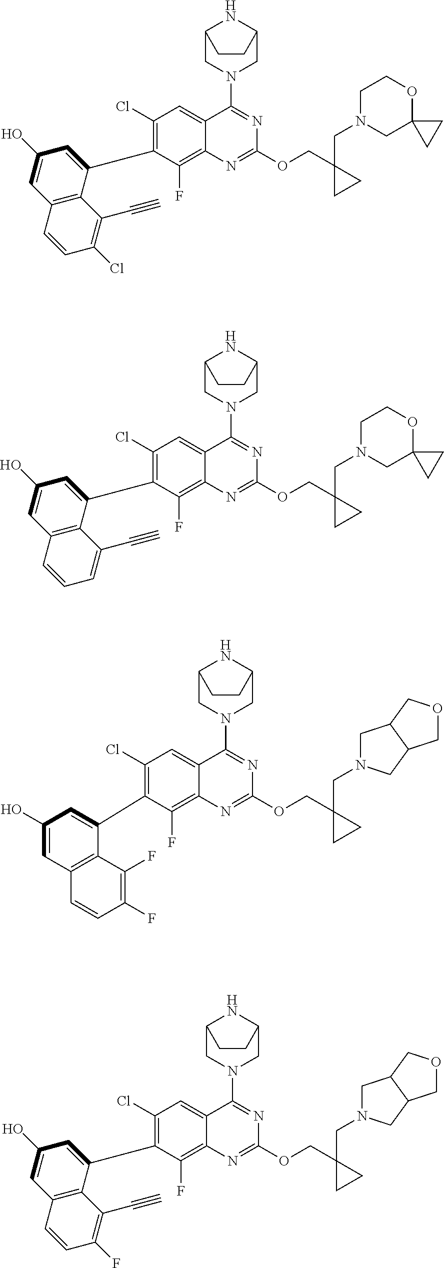

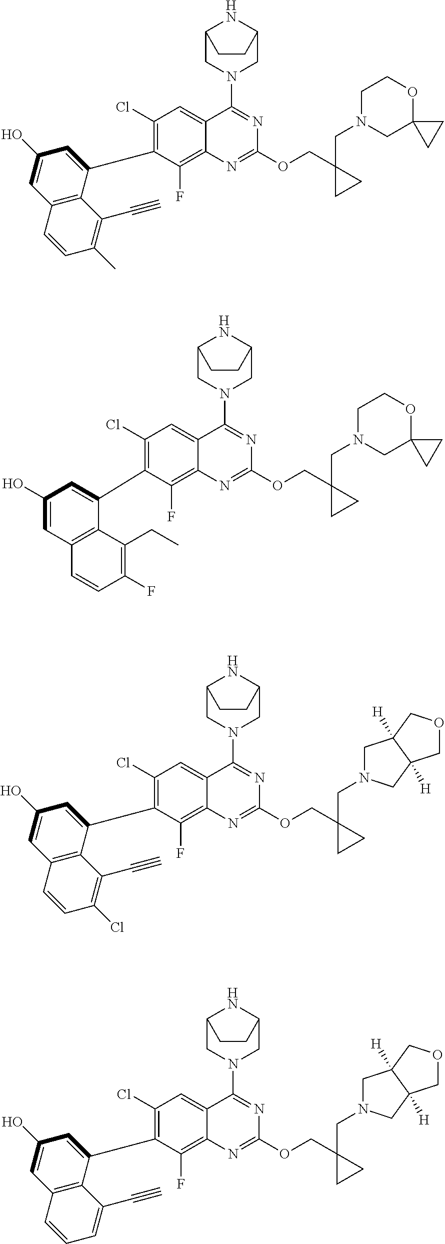

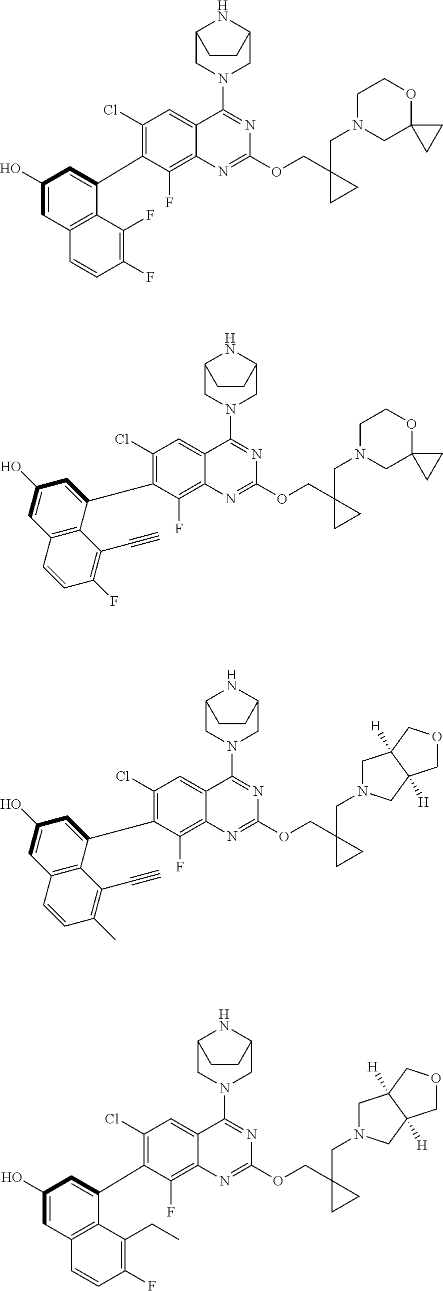

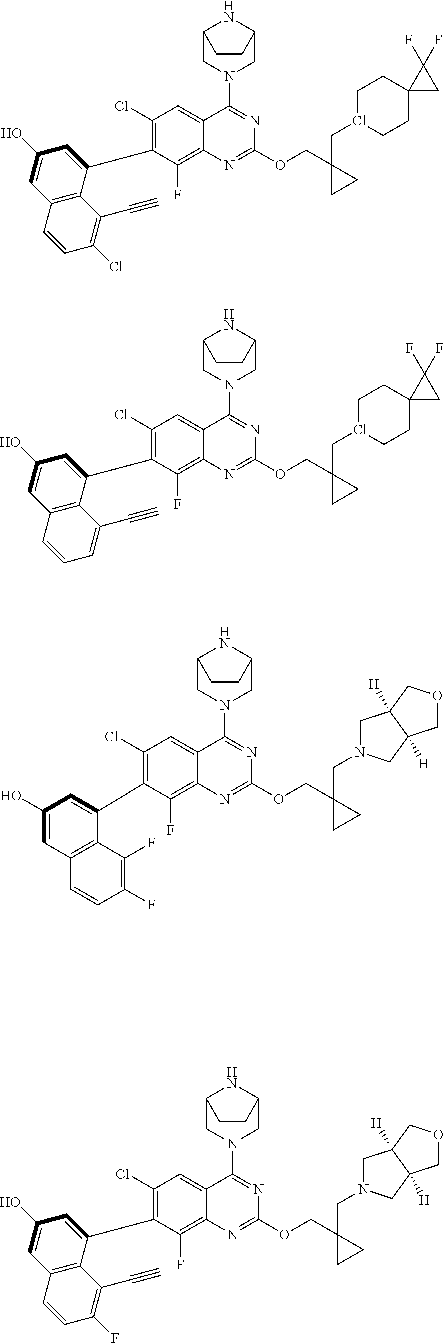

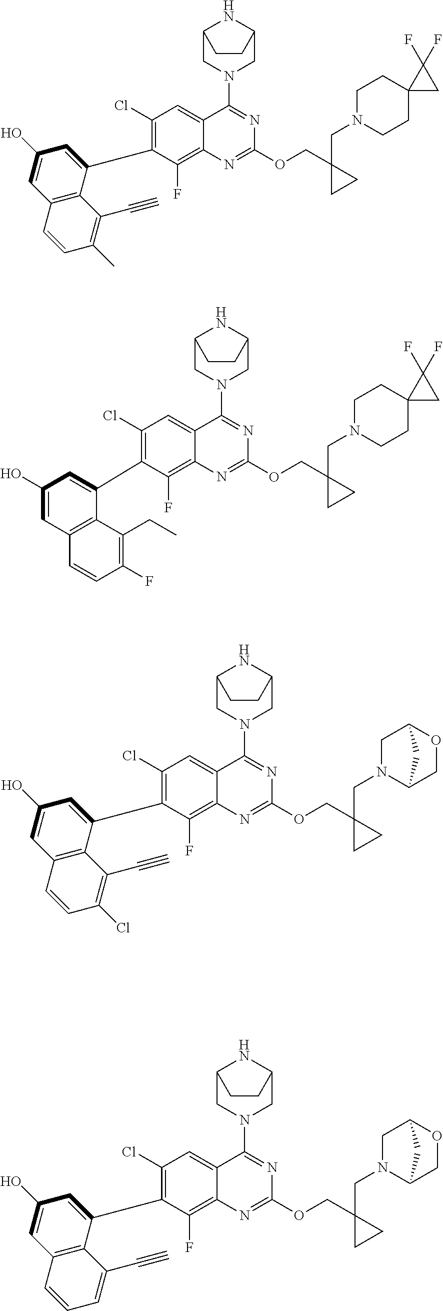

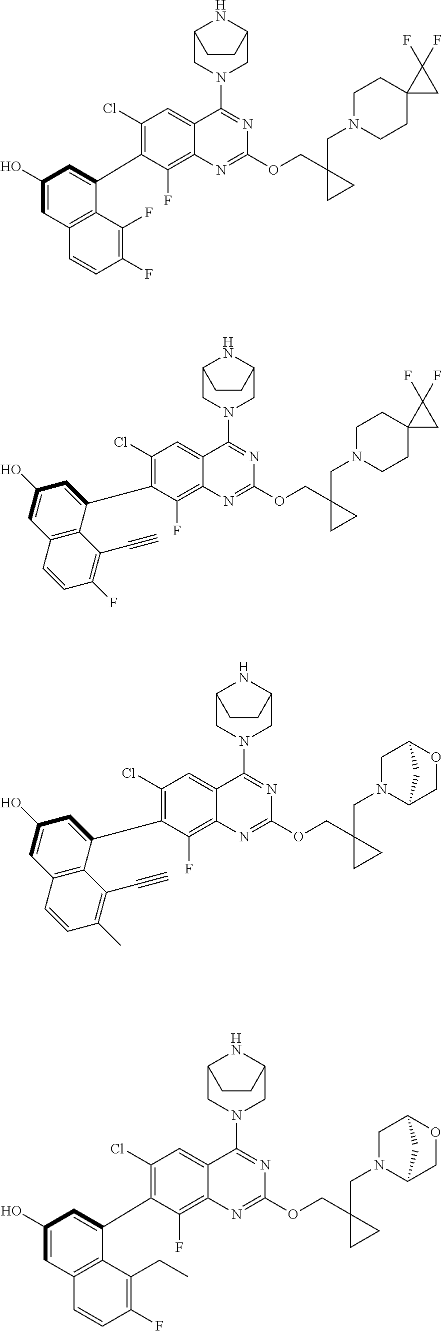

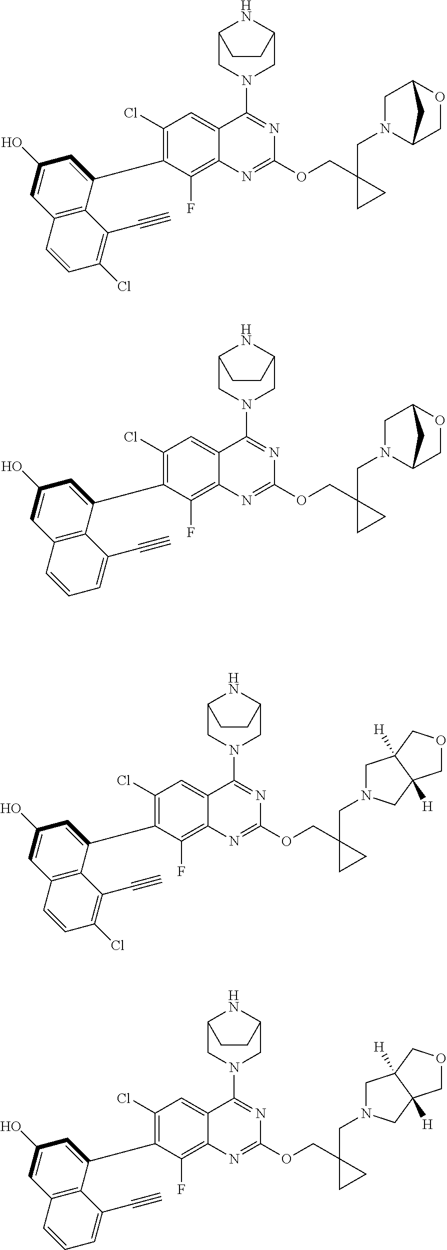

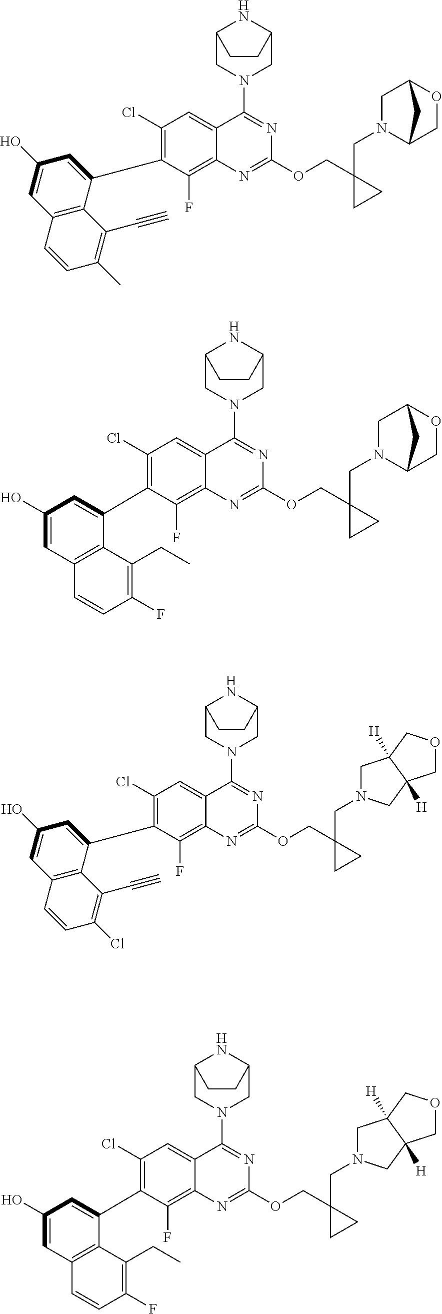

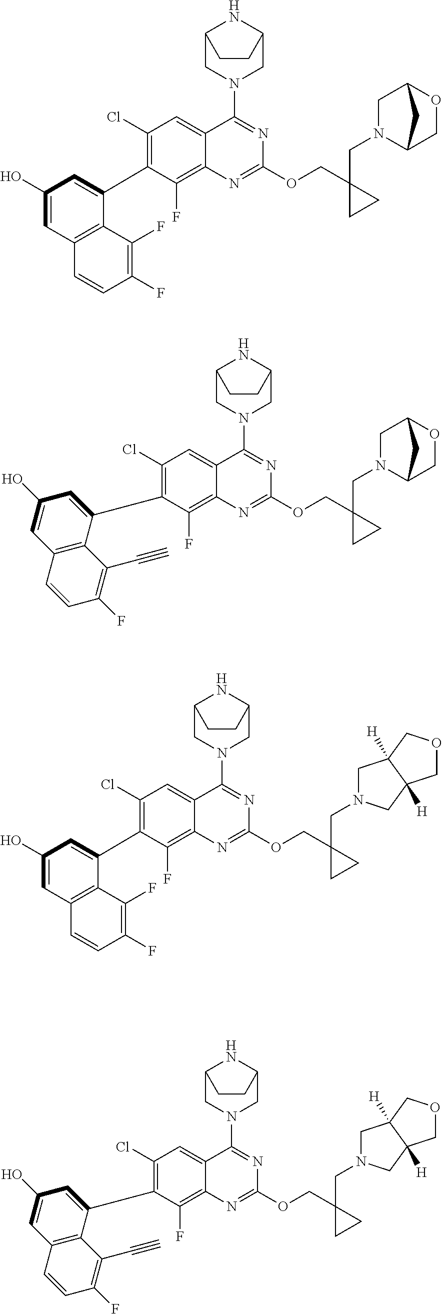

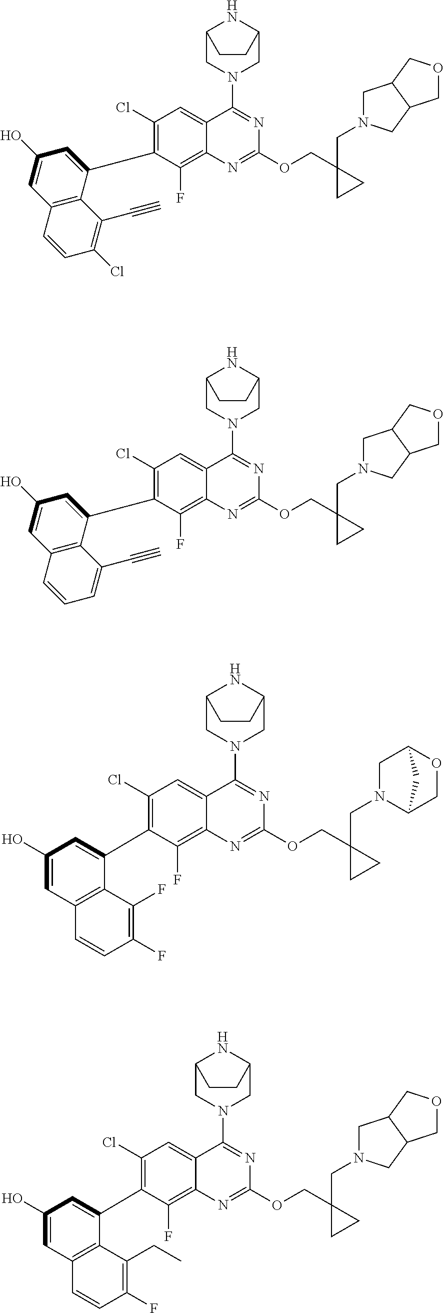

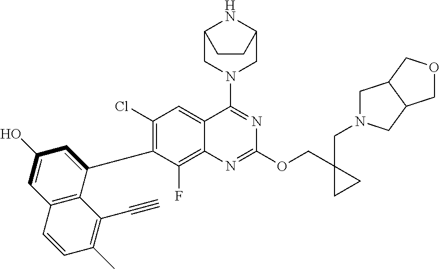

The following compounds could be synthesized with reference to the synthesis procedures of Example 39 and Example 43:

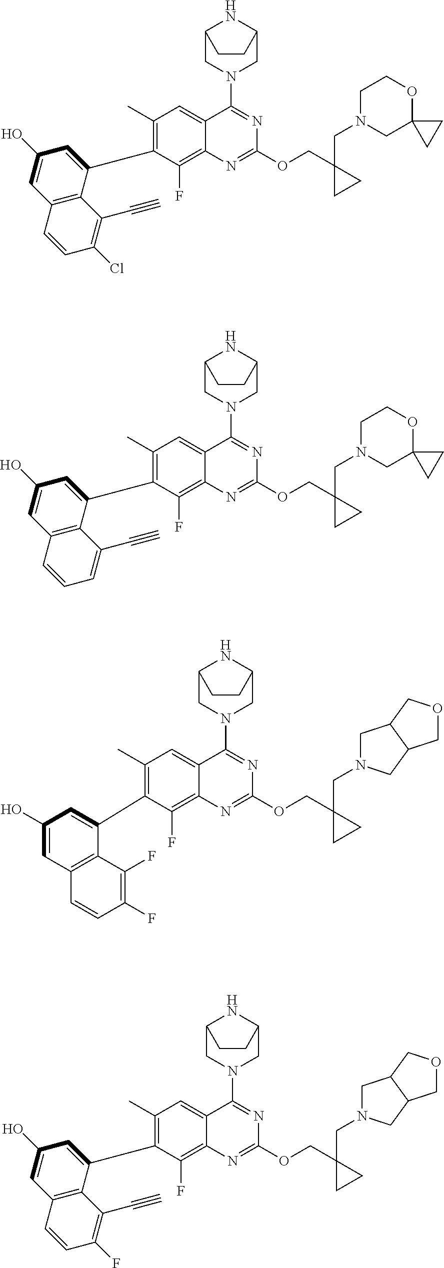

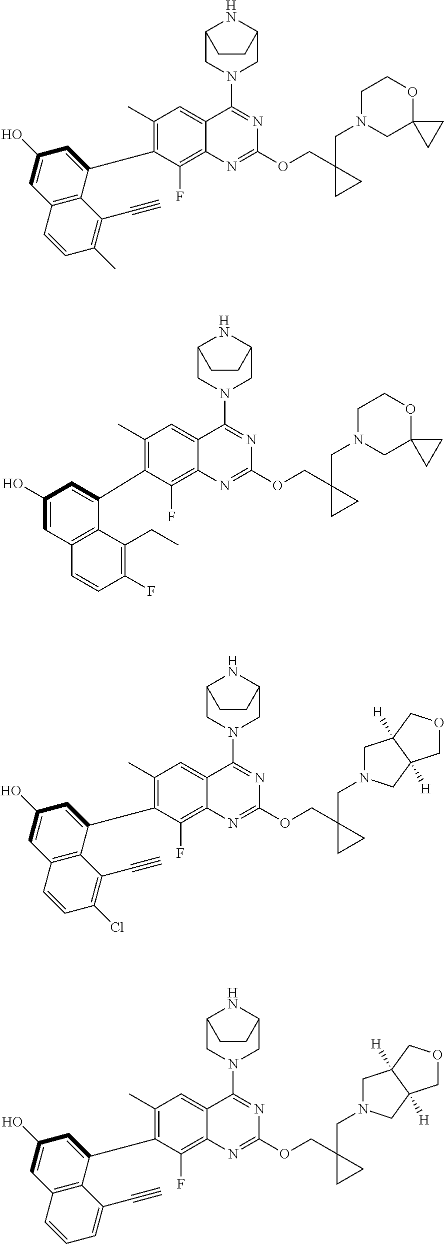

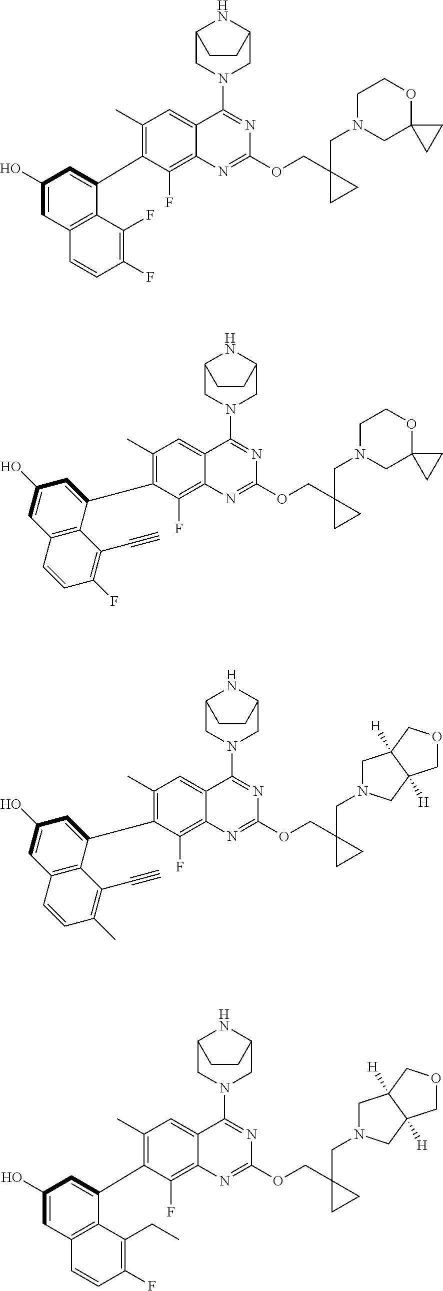

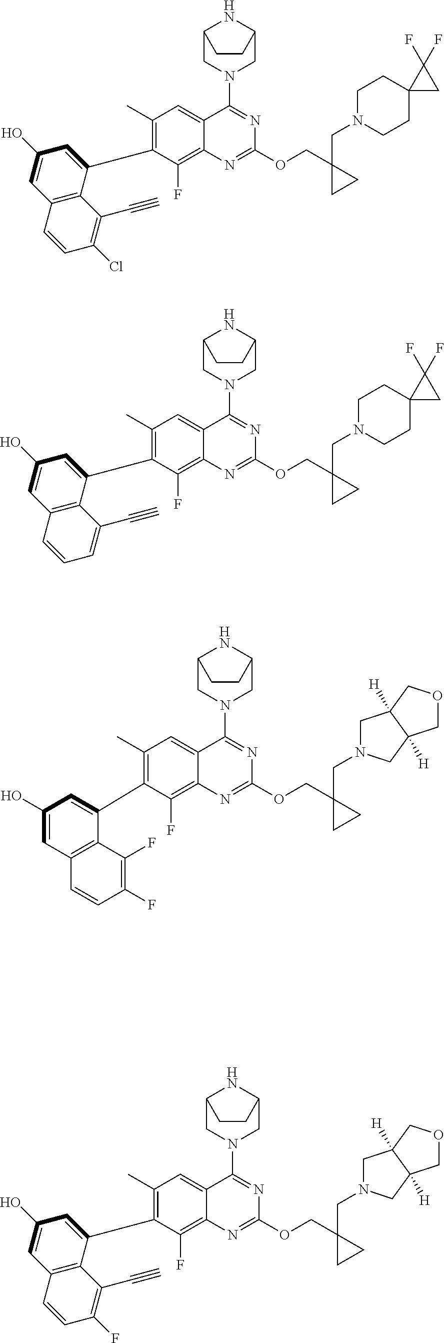

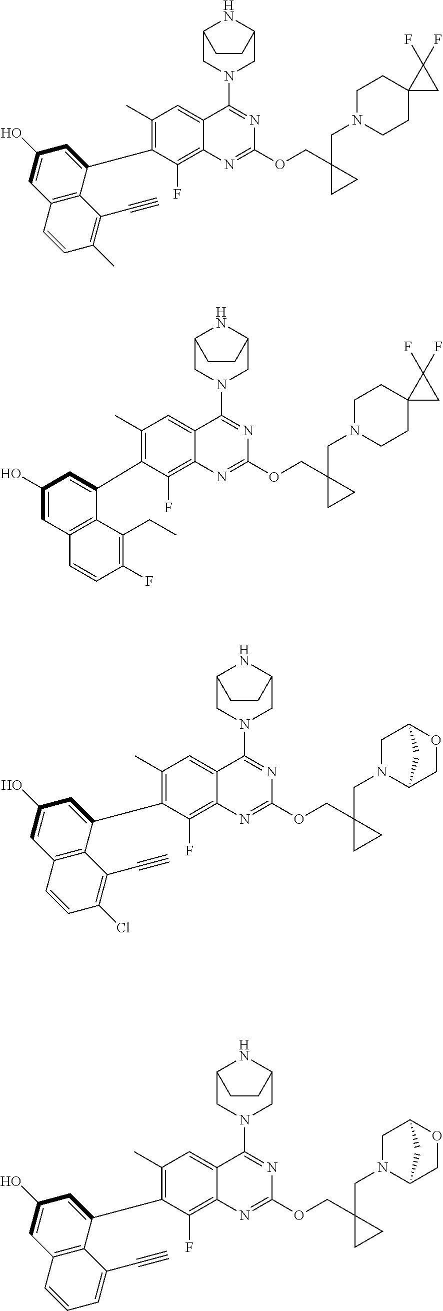

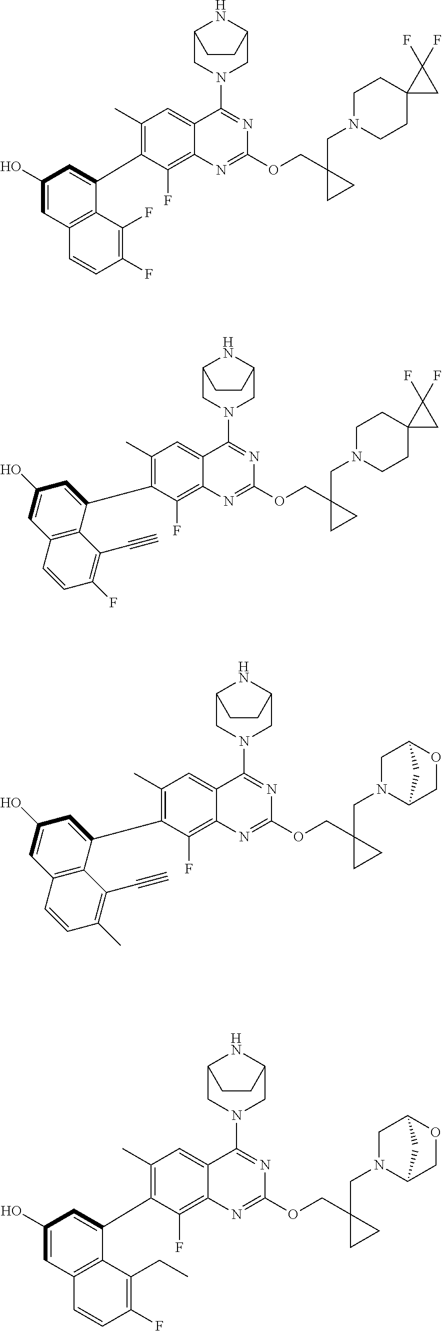

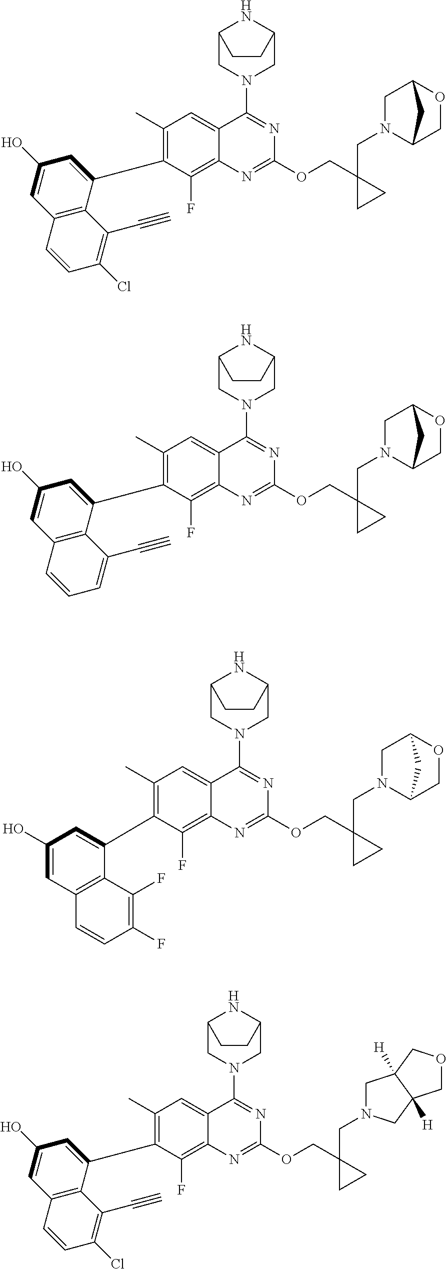

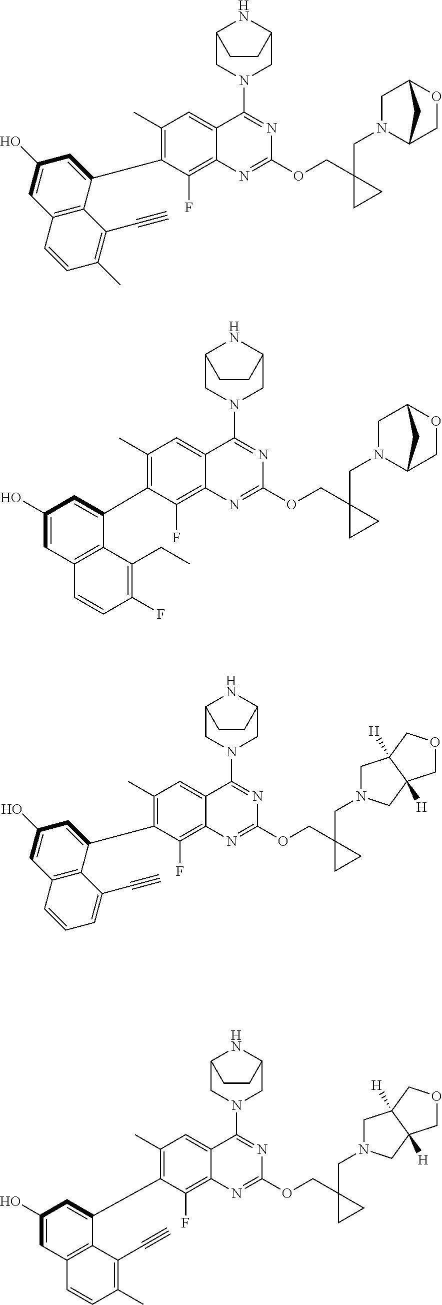

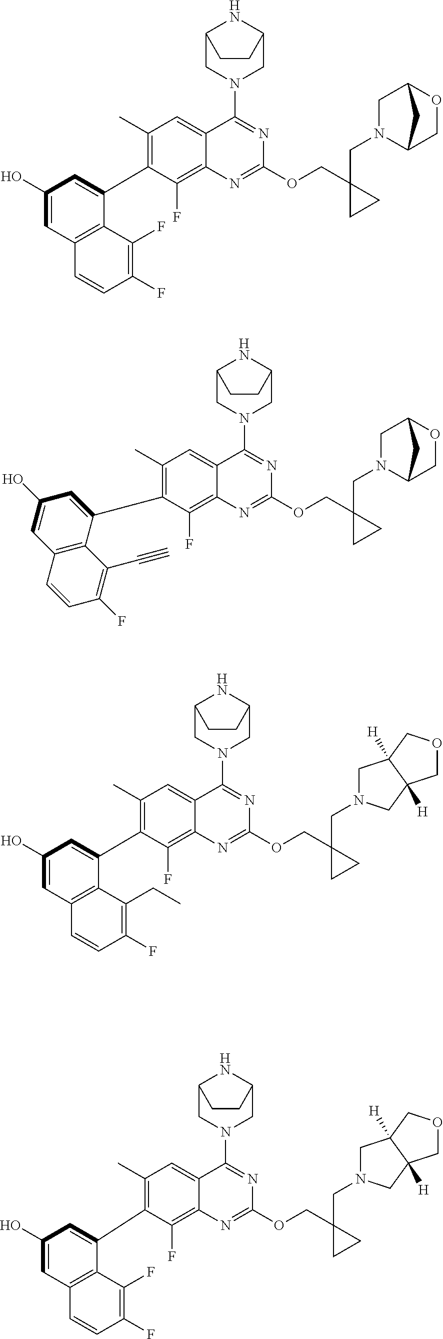

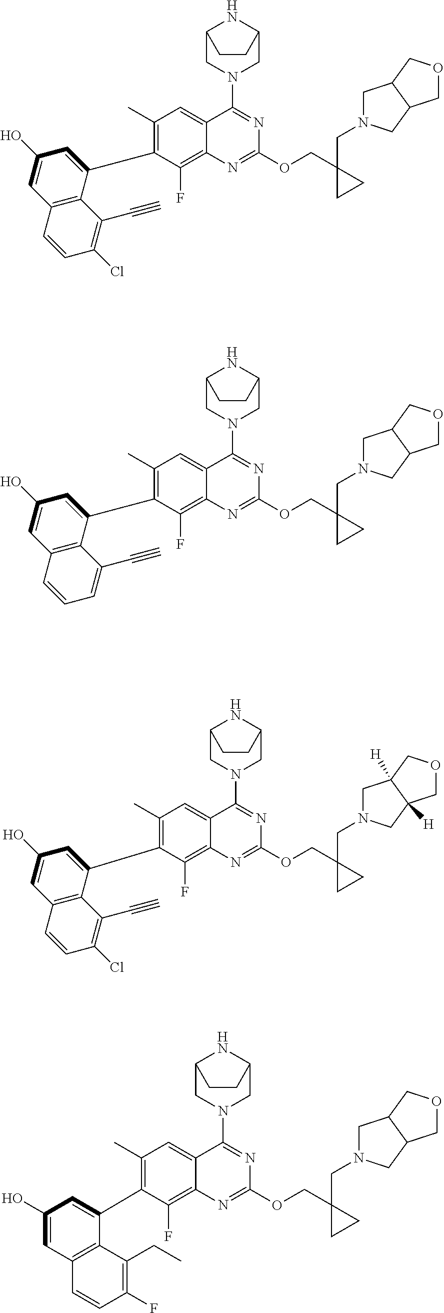

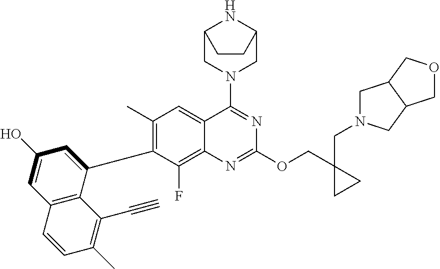

The following compounds could be synthesized with reference to the synthesis procedures of Example 42 and Example 43:

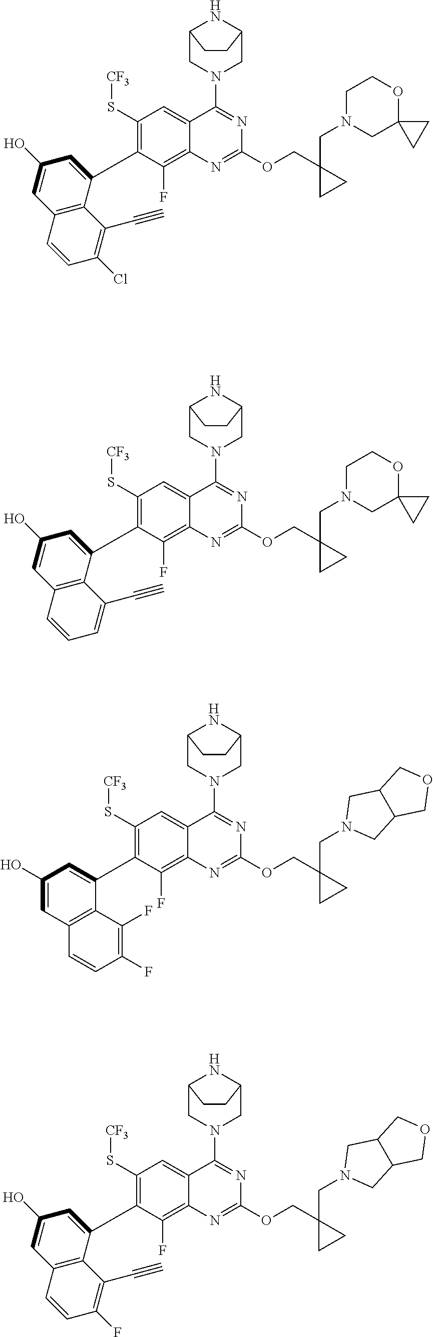

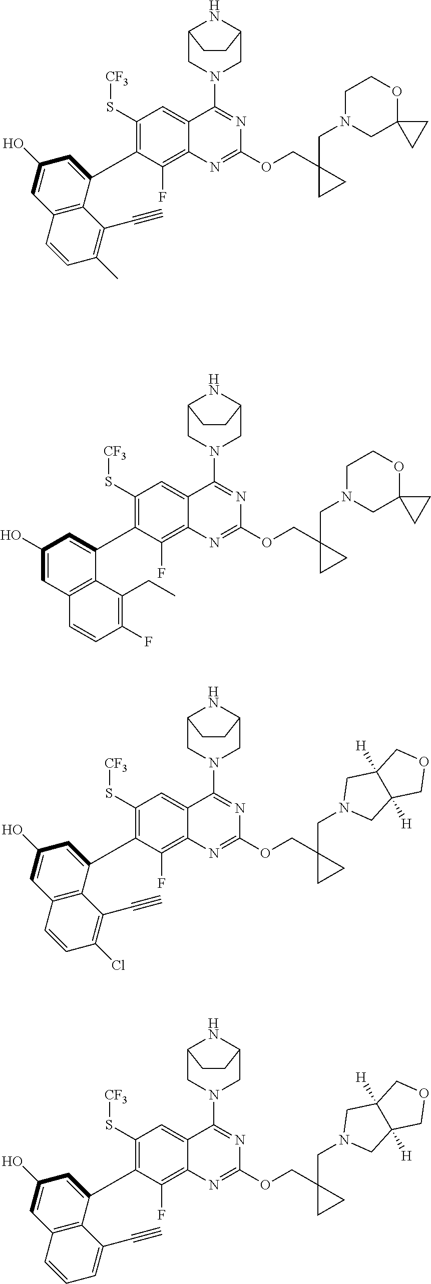

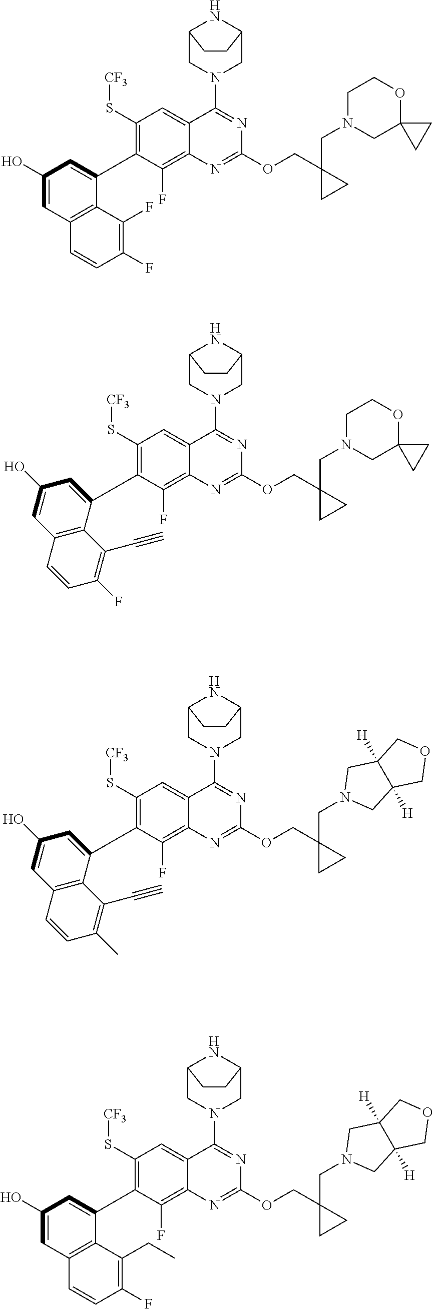

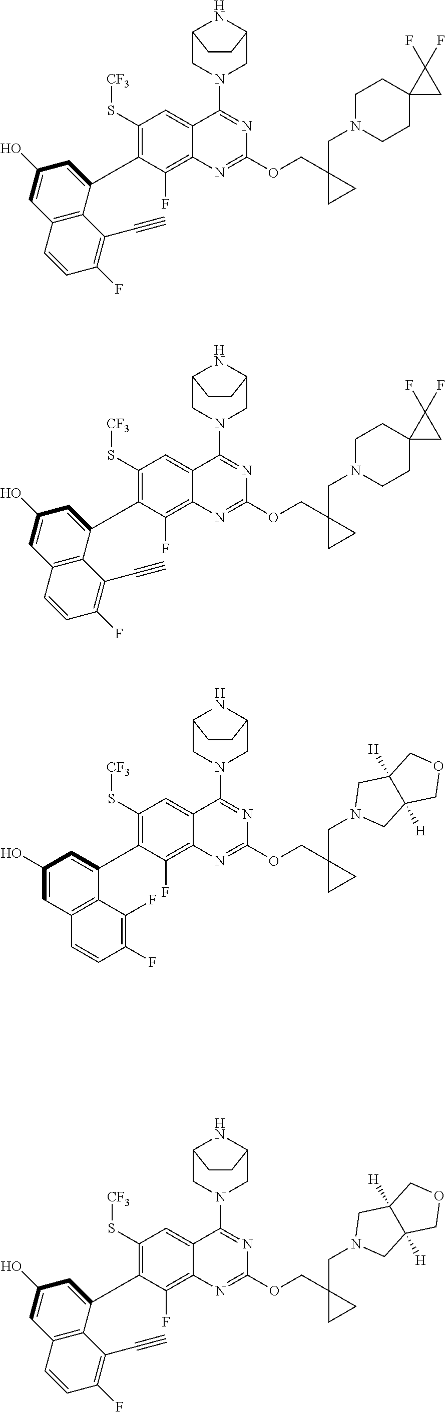

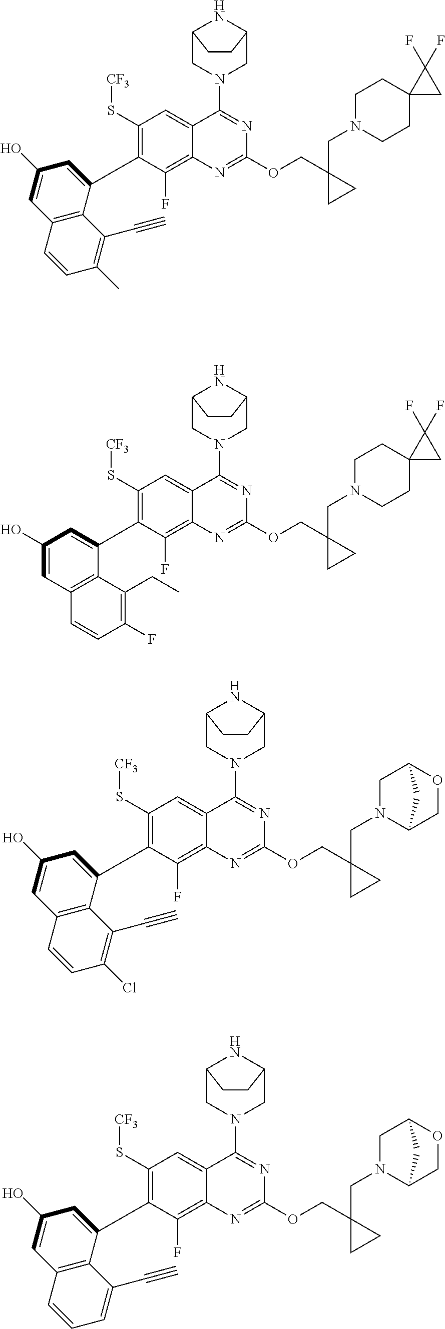

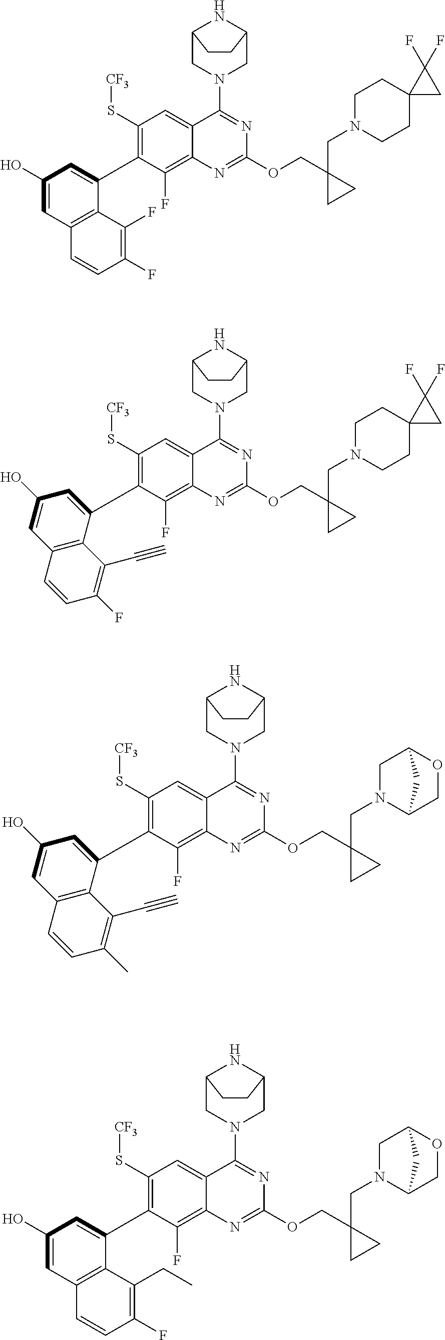

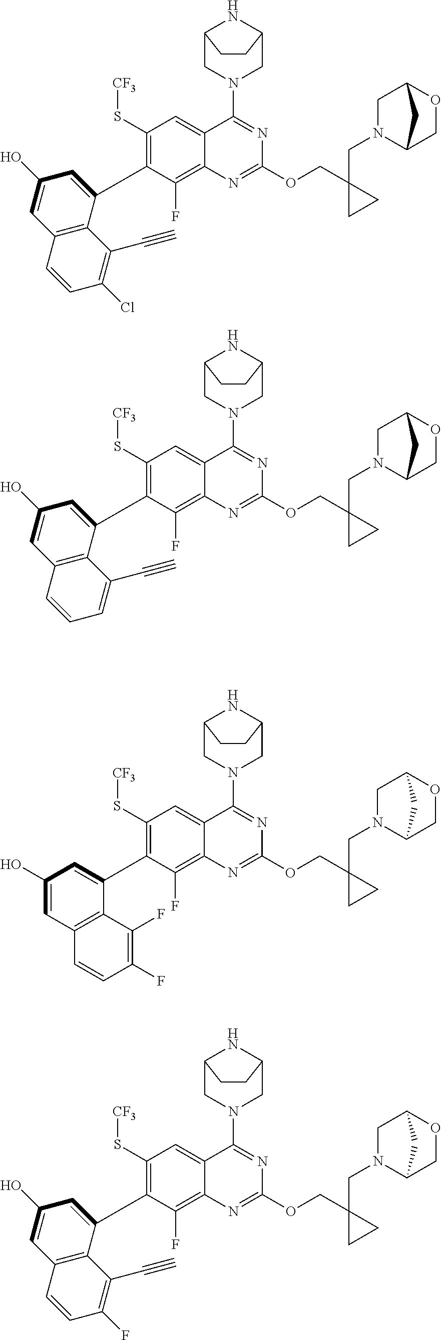

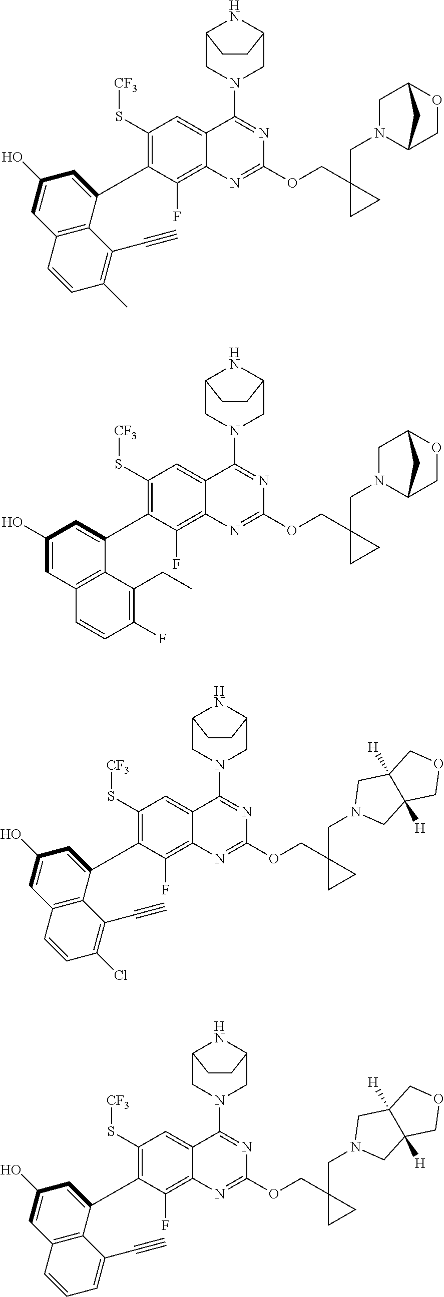

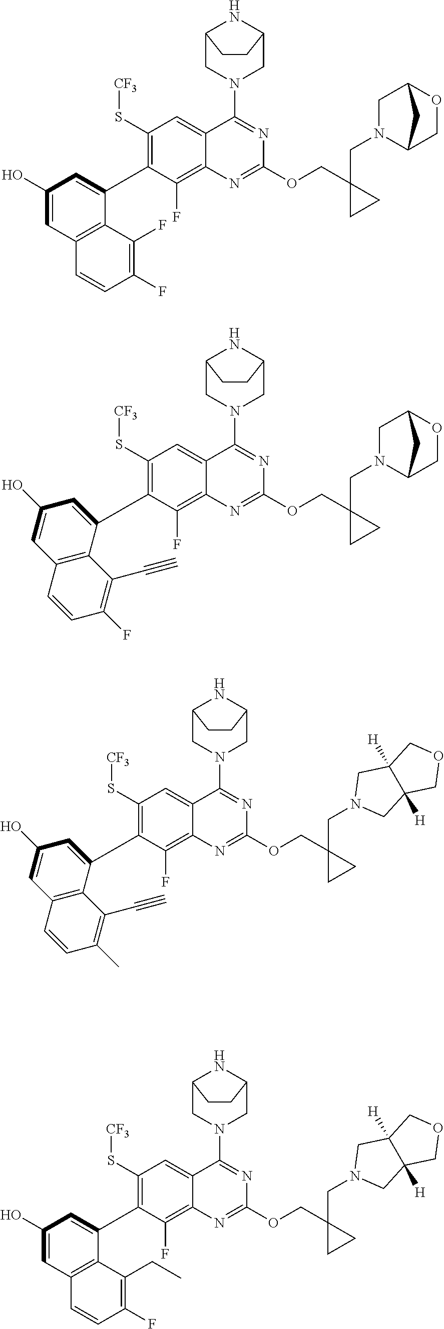

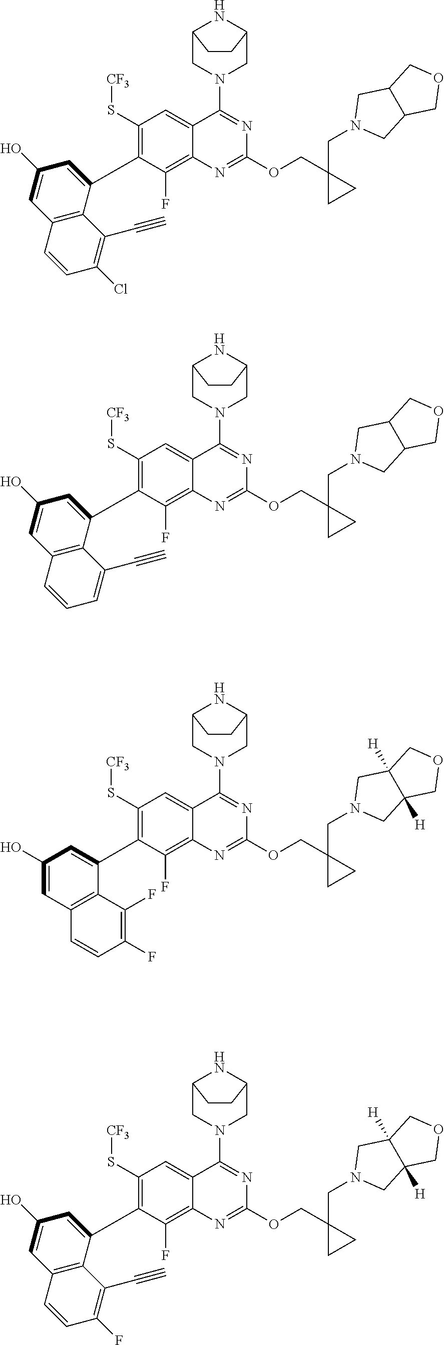

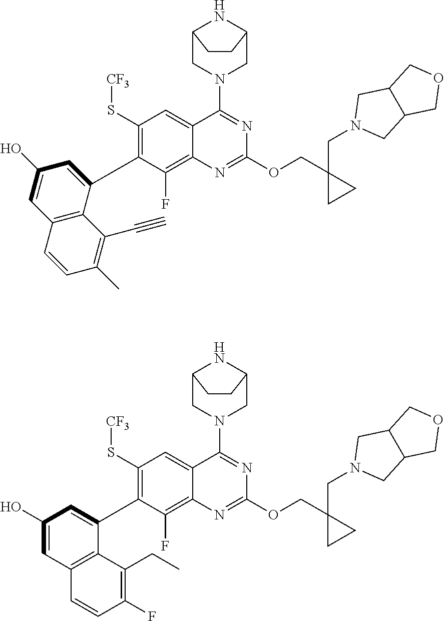

The following compounds could be synthesized with reference to the synthesis procedures of Example 2 and Example 43:

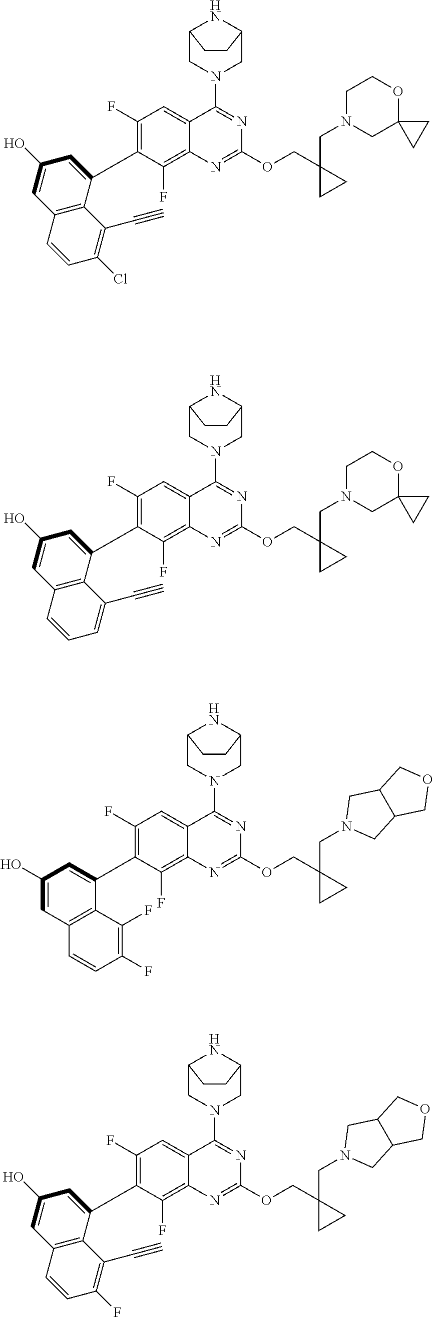

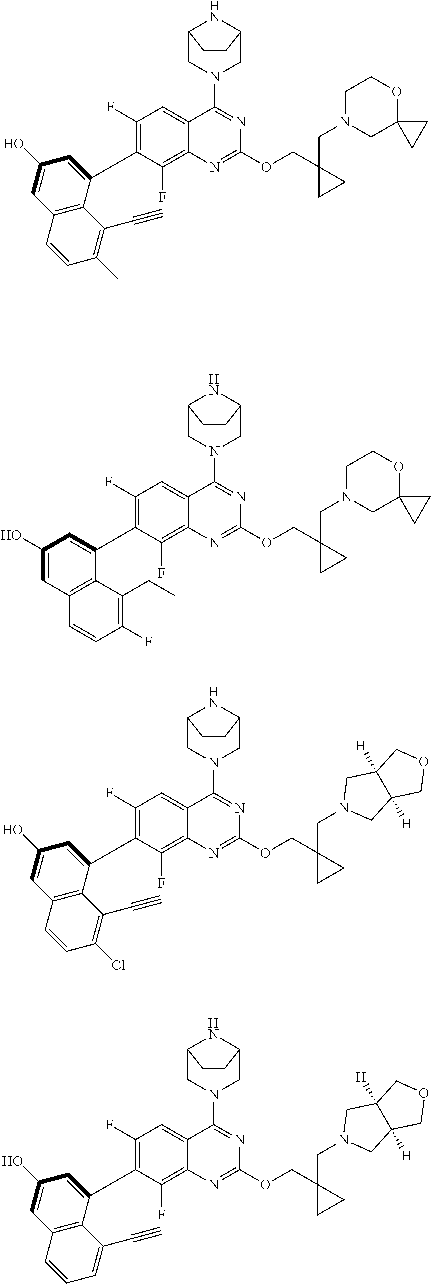

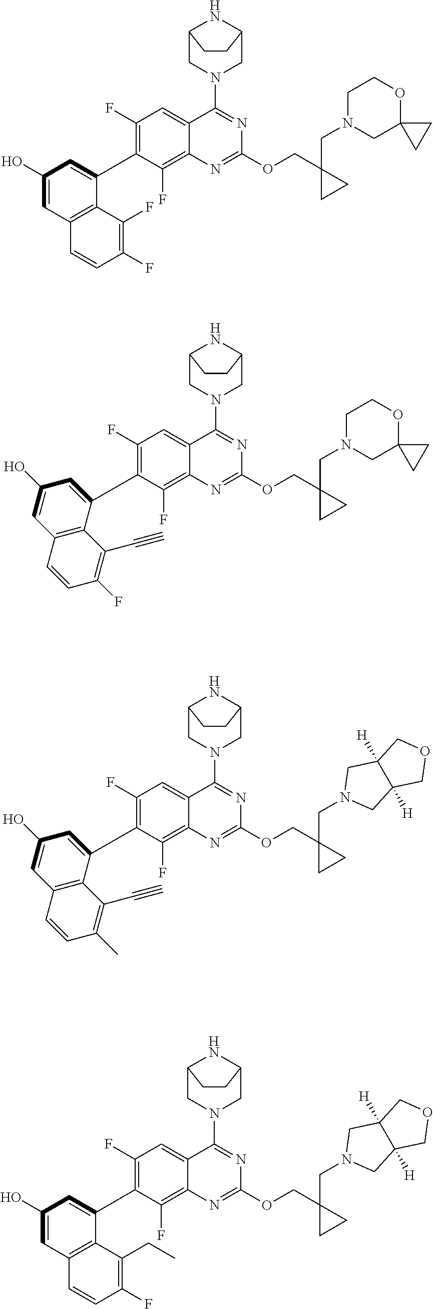

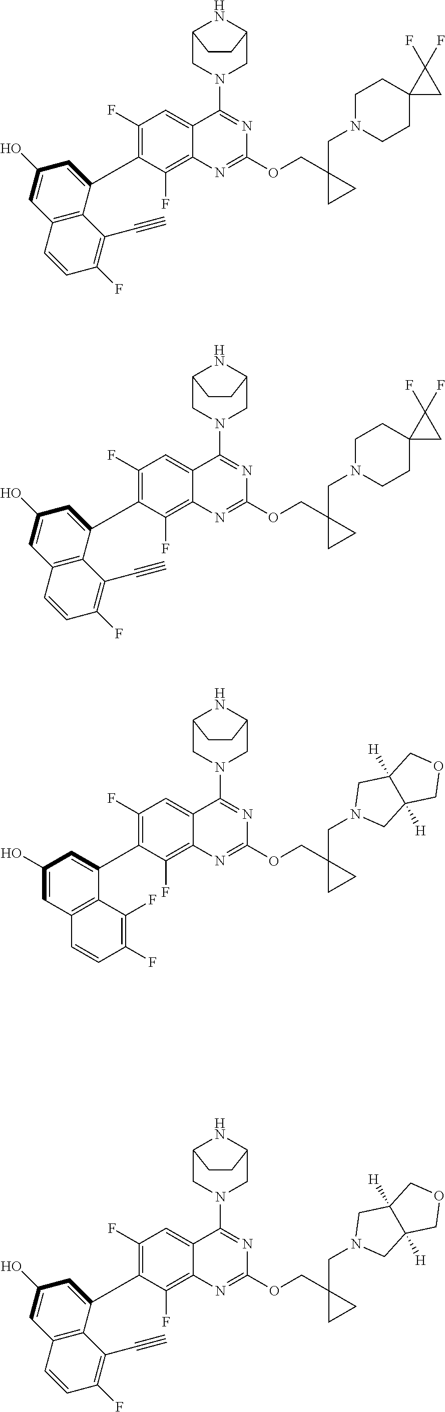

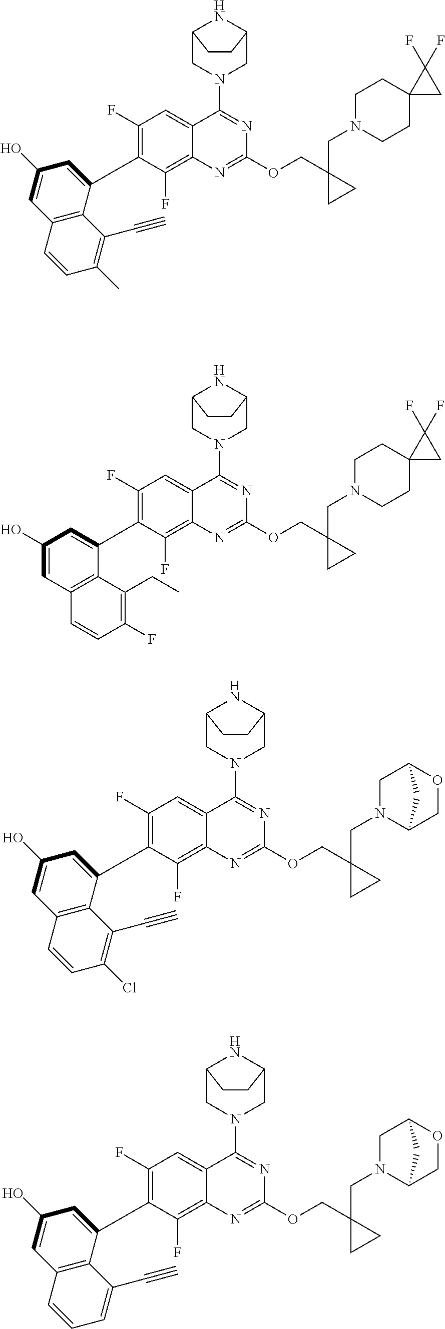

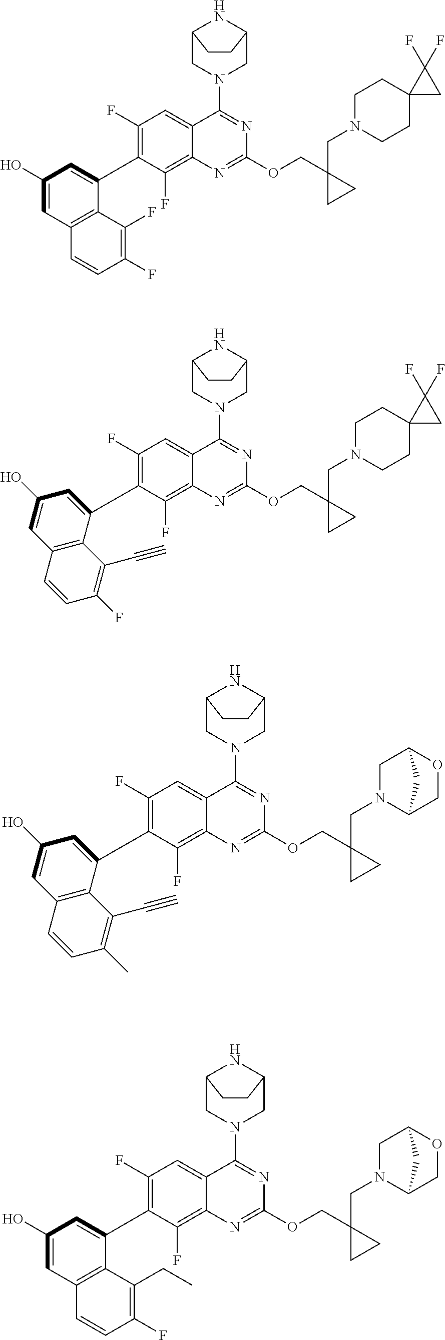

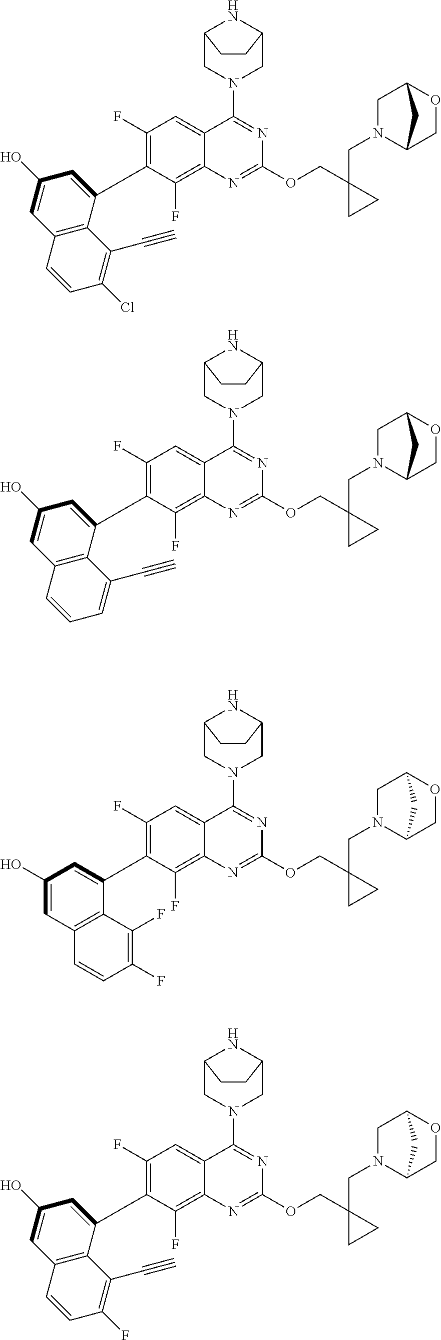

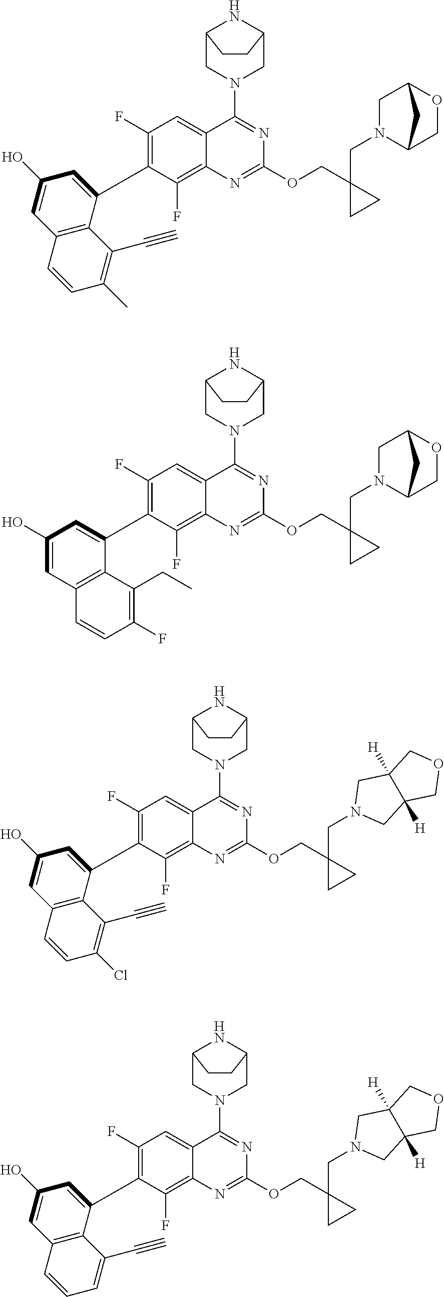

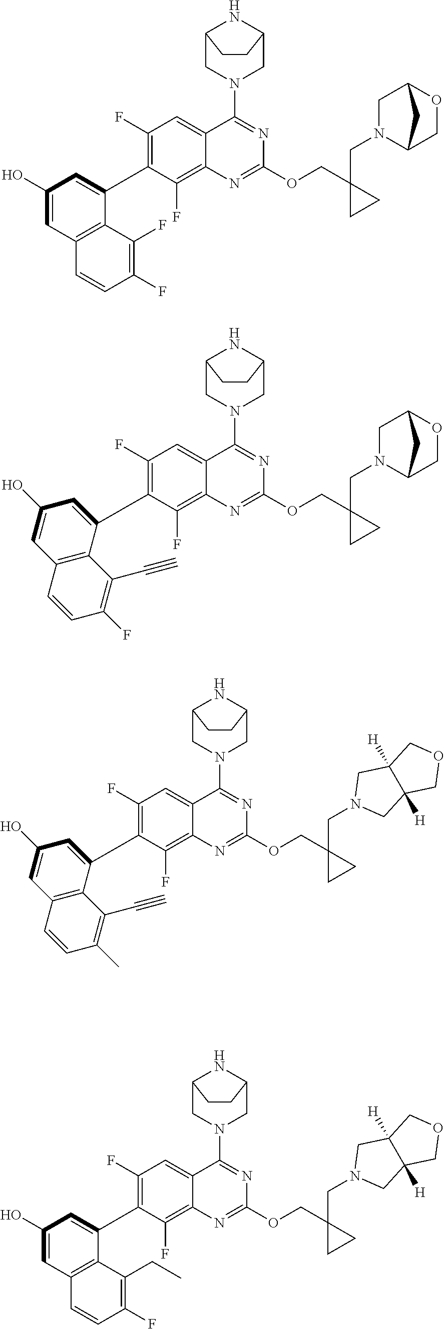

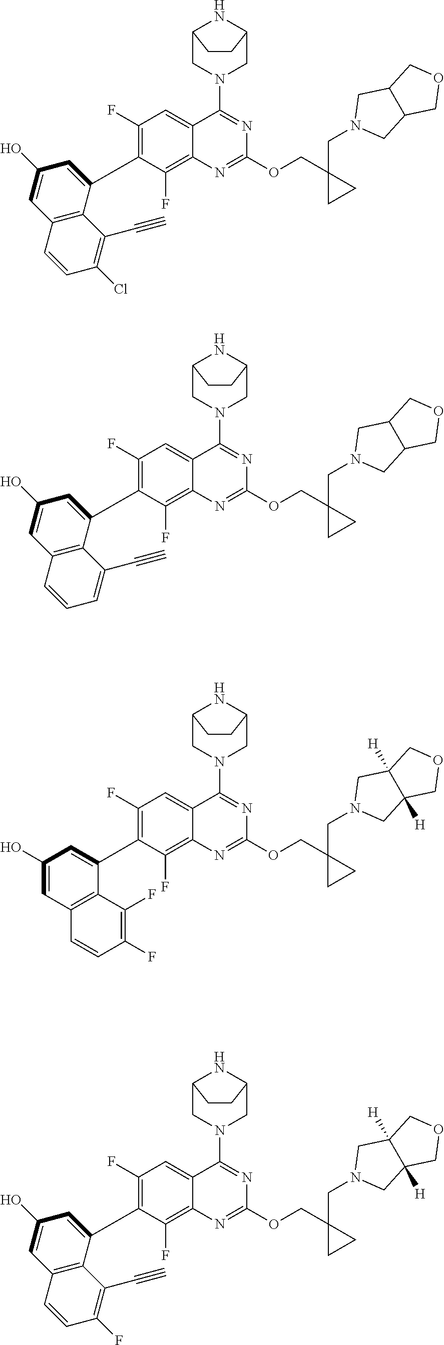

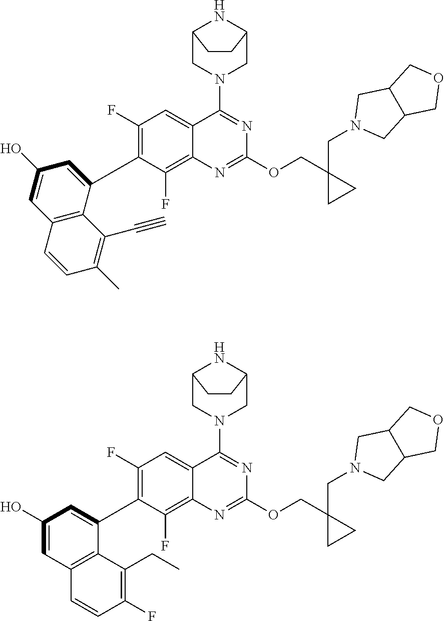

The following compounds could be synthesized with reference to the synthesis procedures of Example 40 and Example 43:

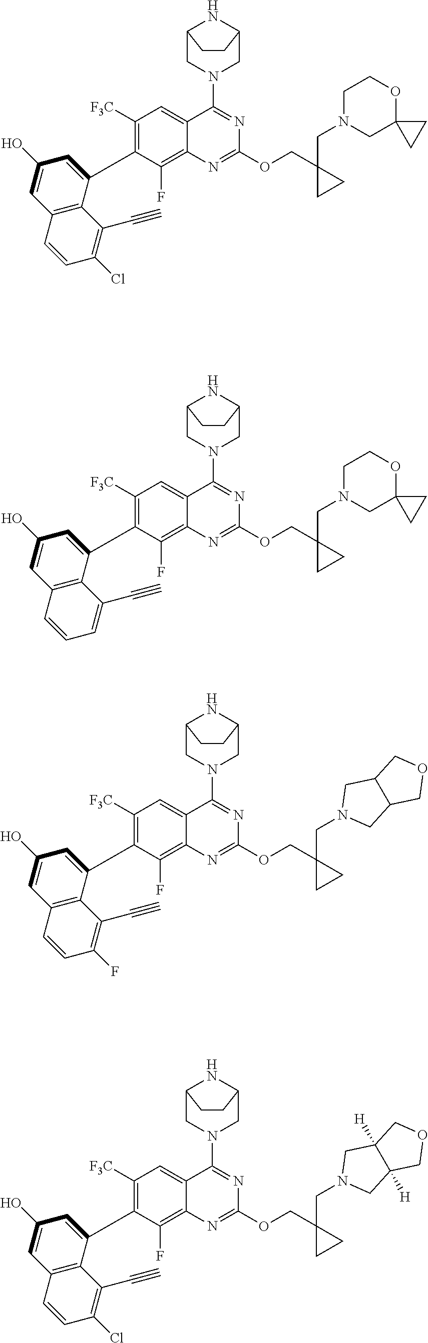

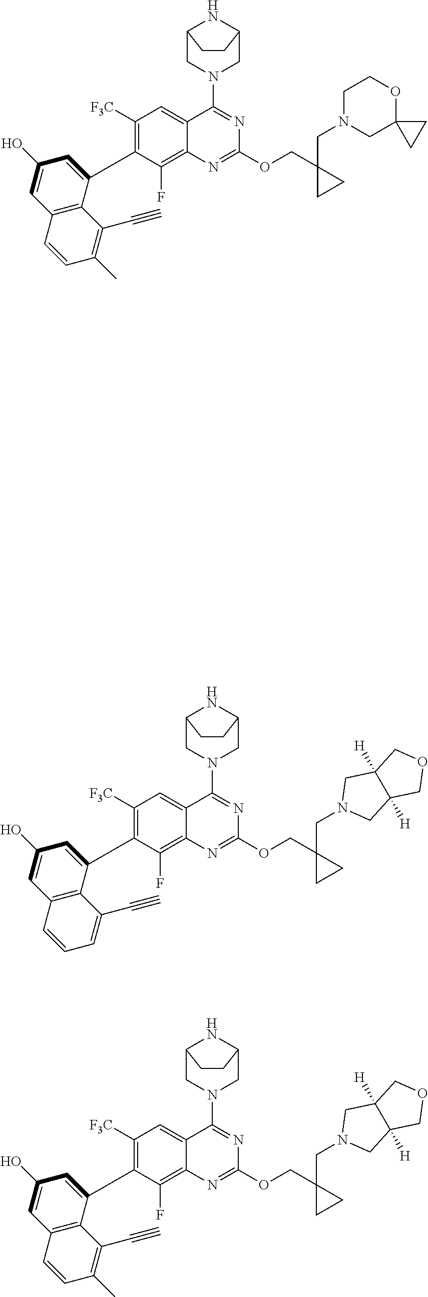

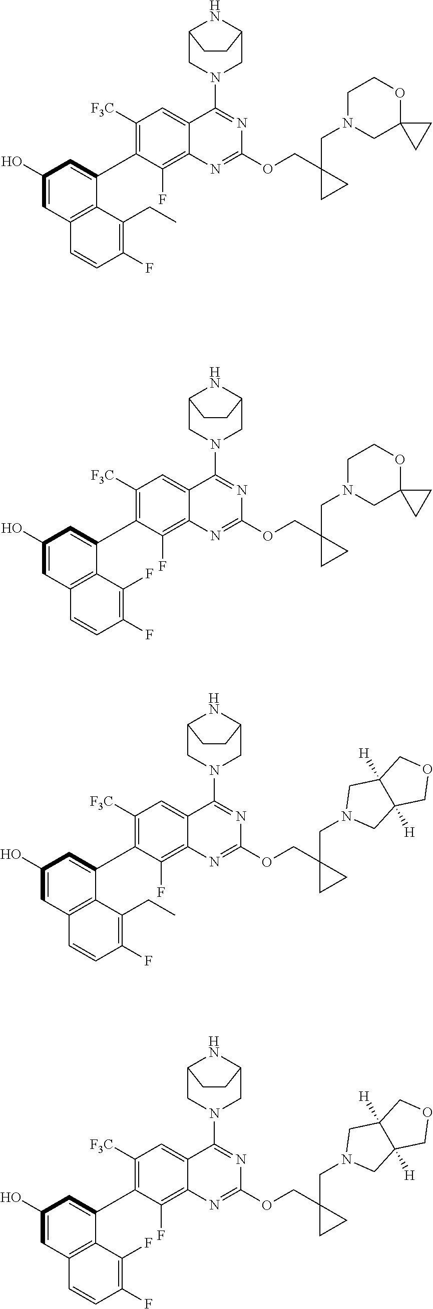

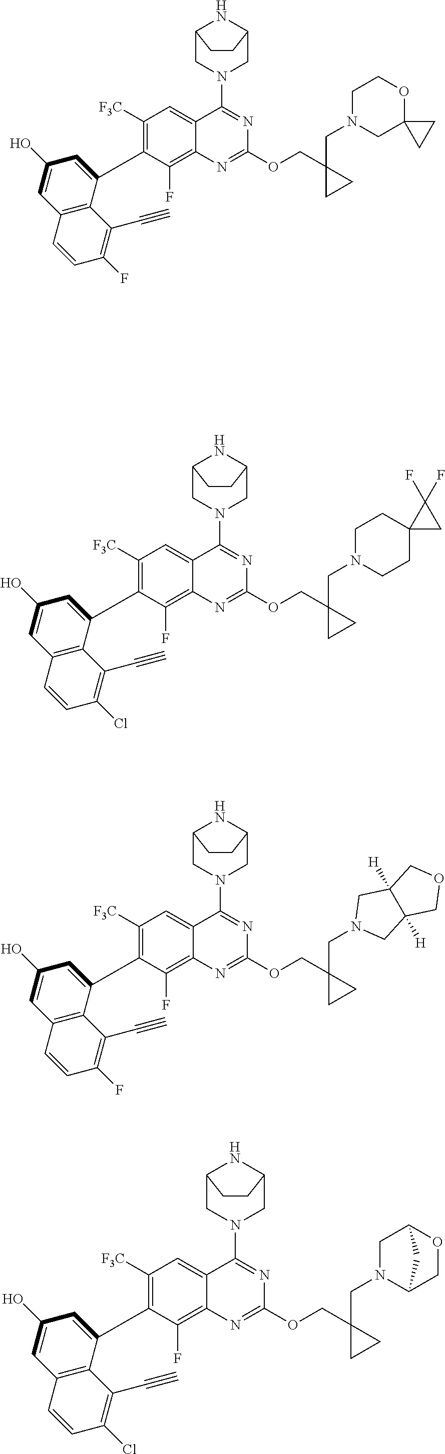

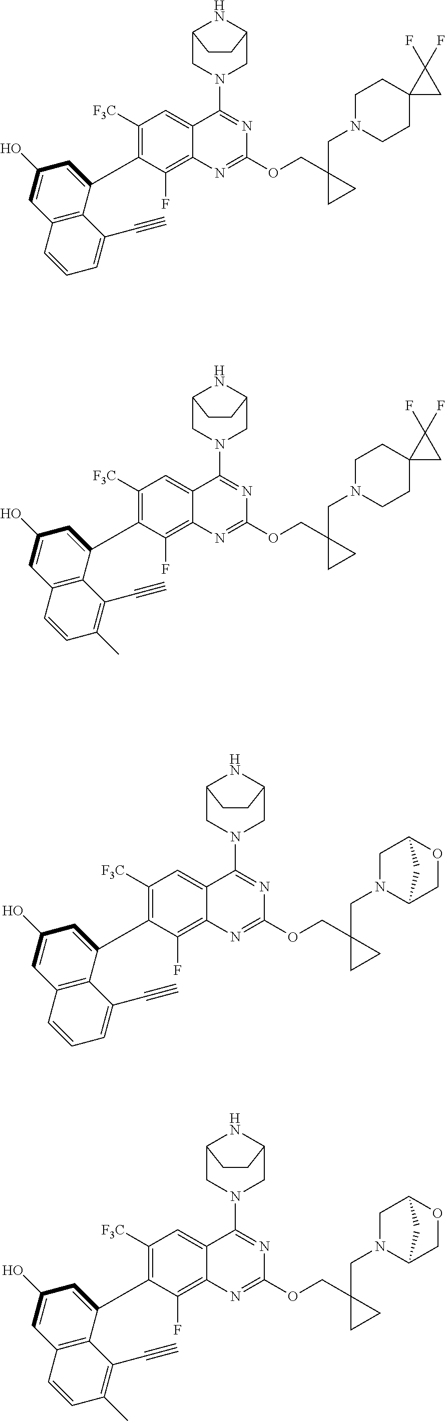

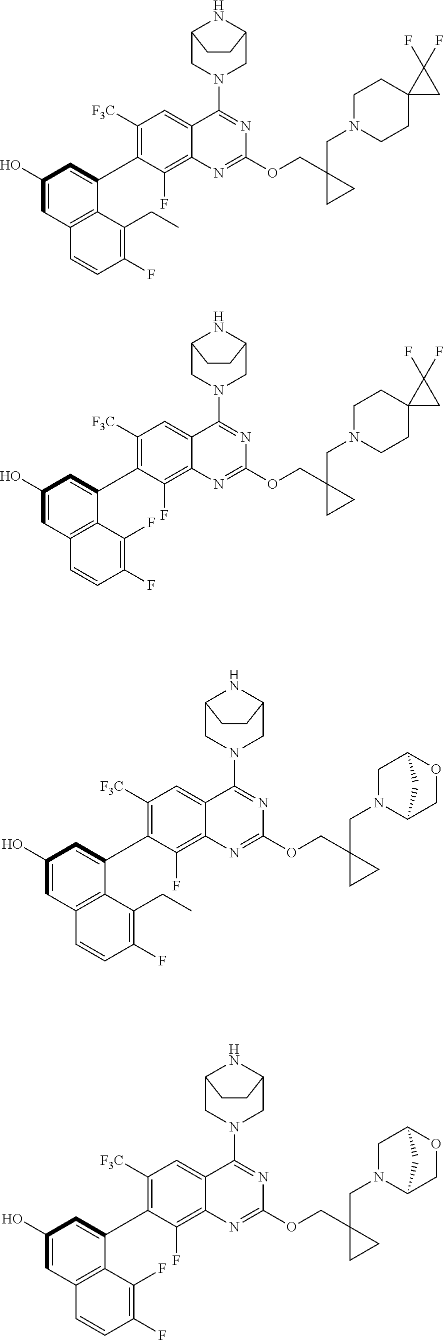

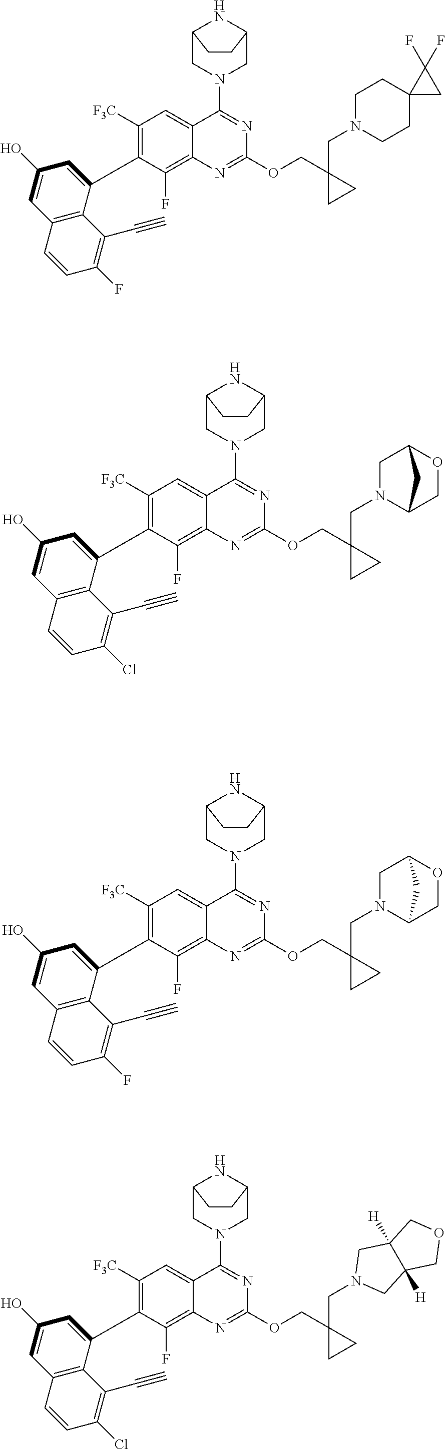

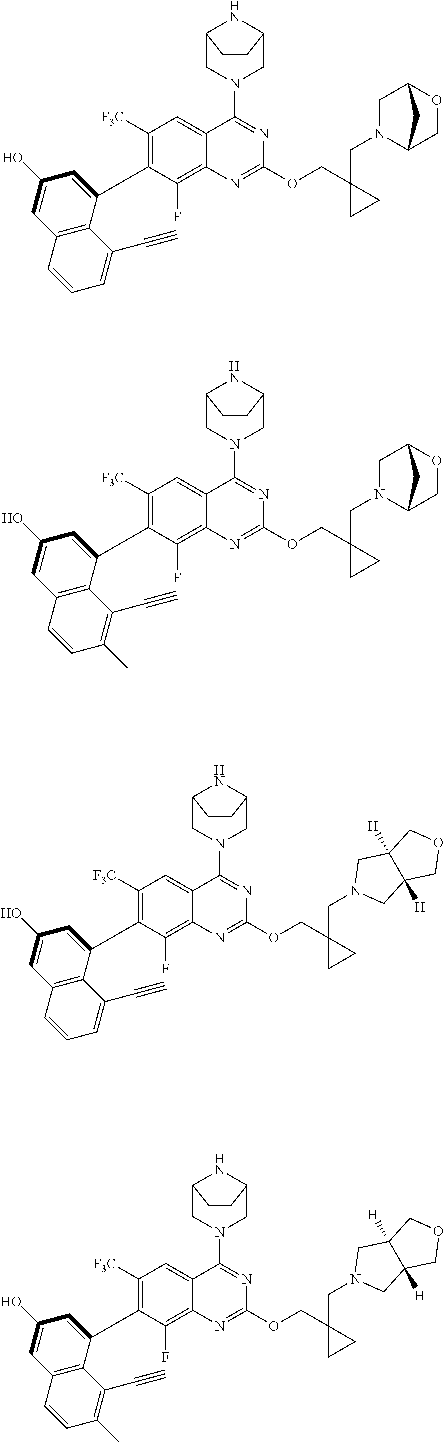

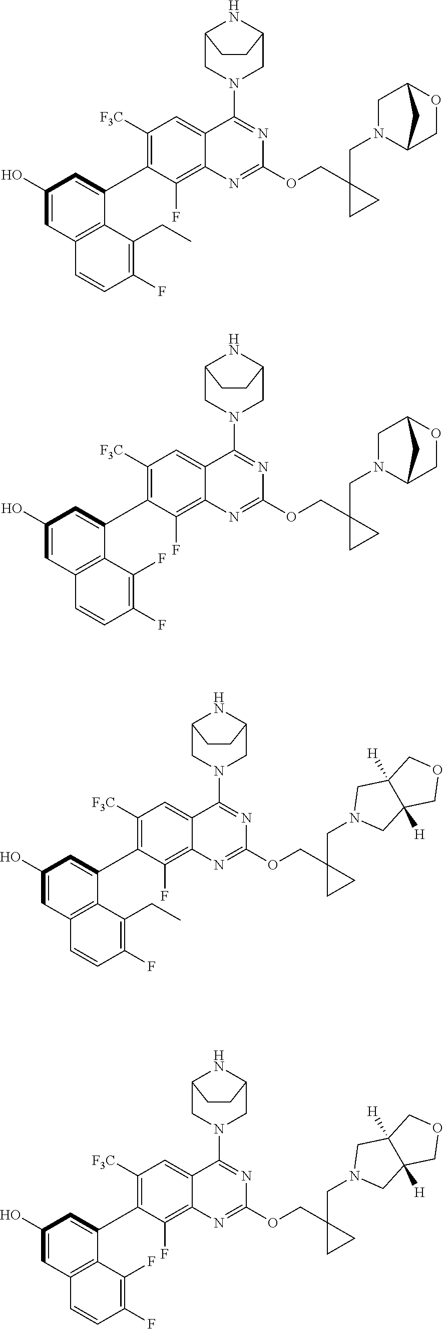

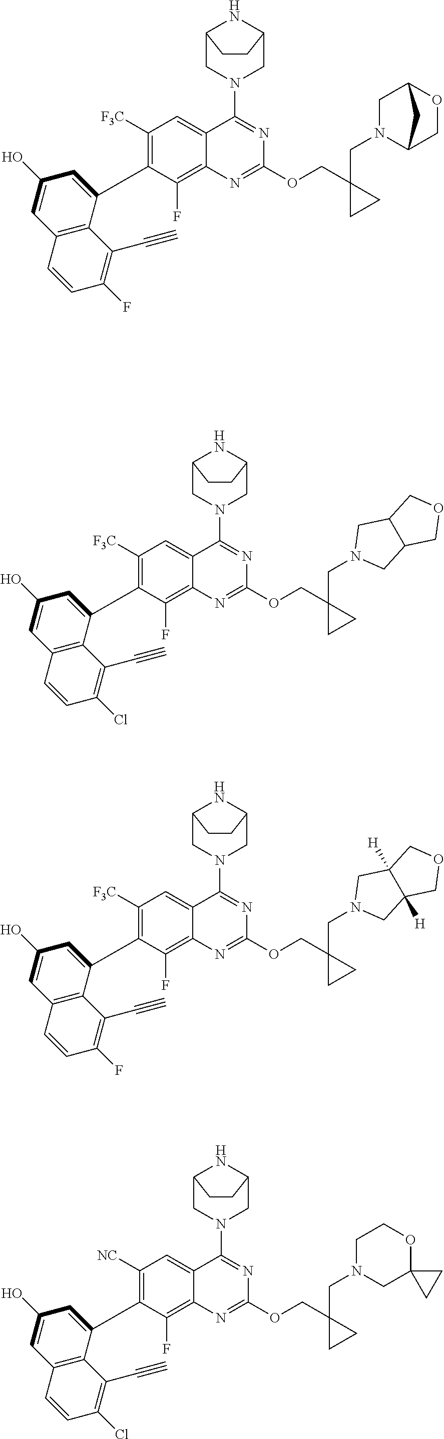

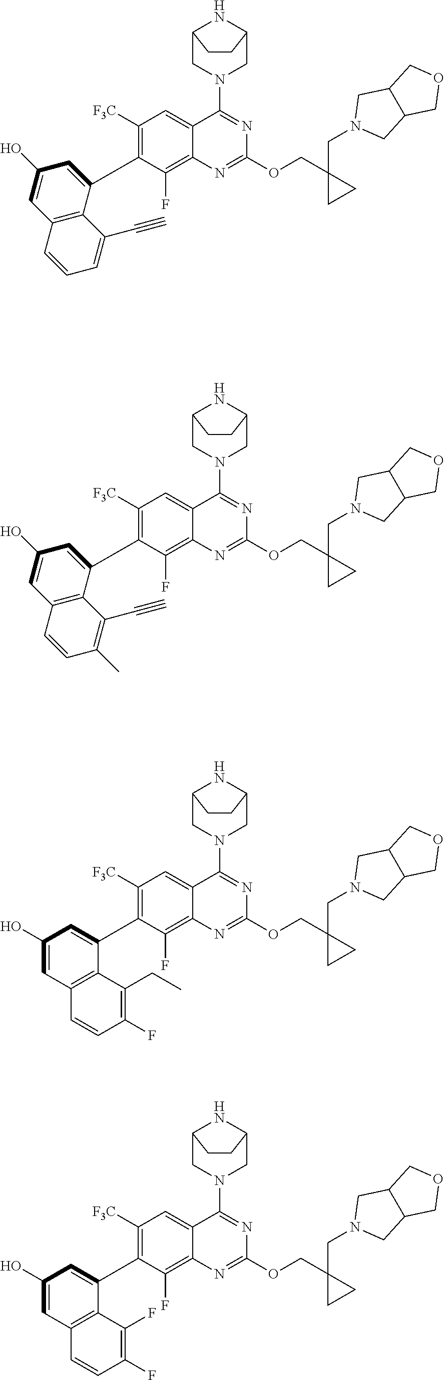

The following compounds could be synthesized with reference to the synthesis procedures of Example 41 and Example 43:

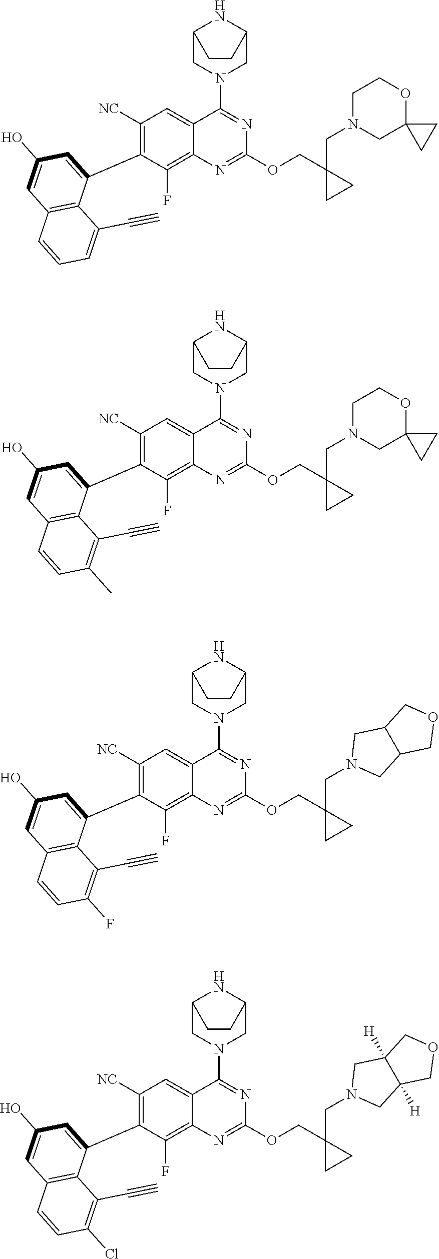

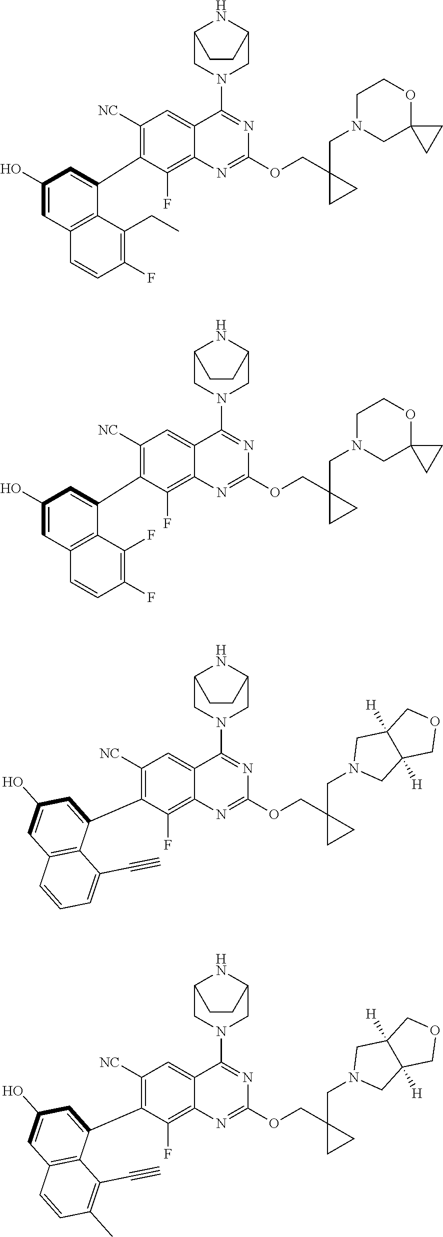

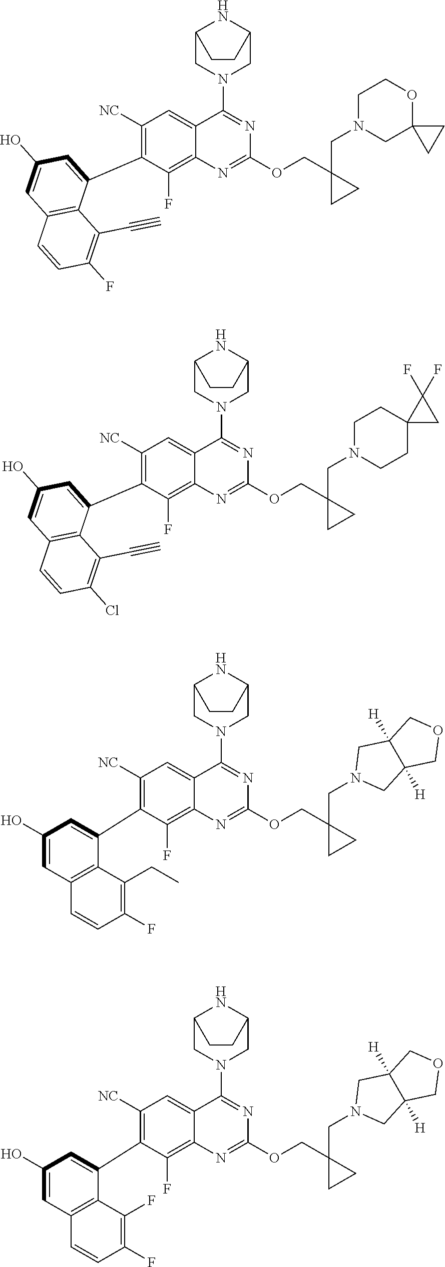

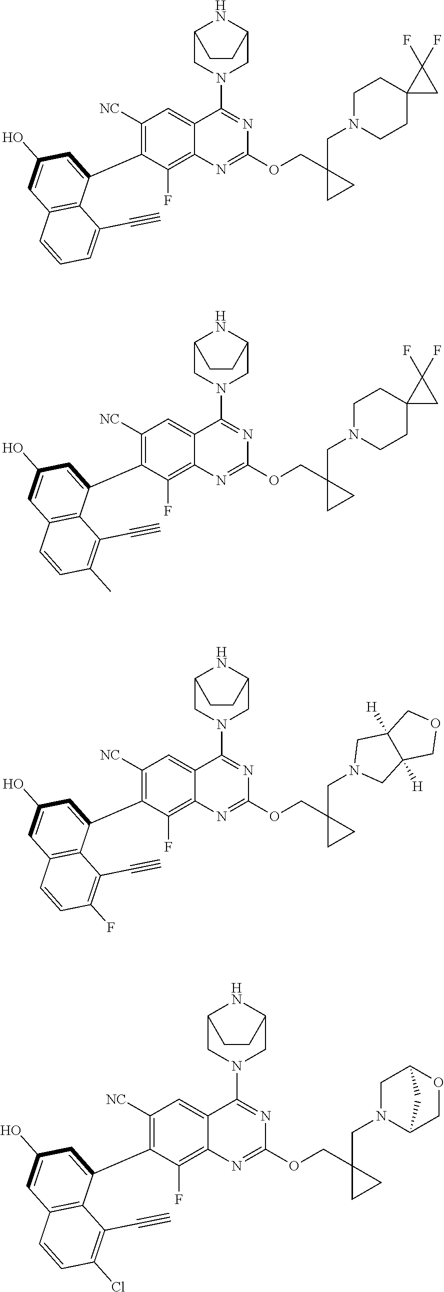

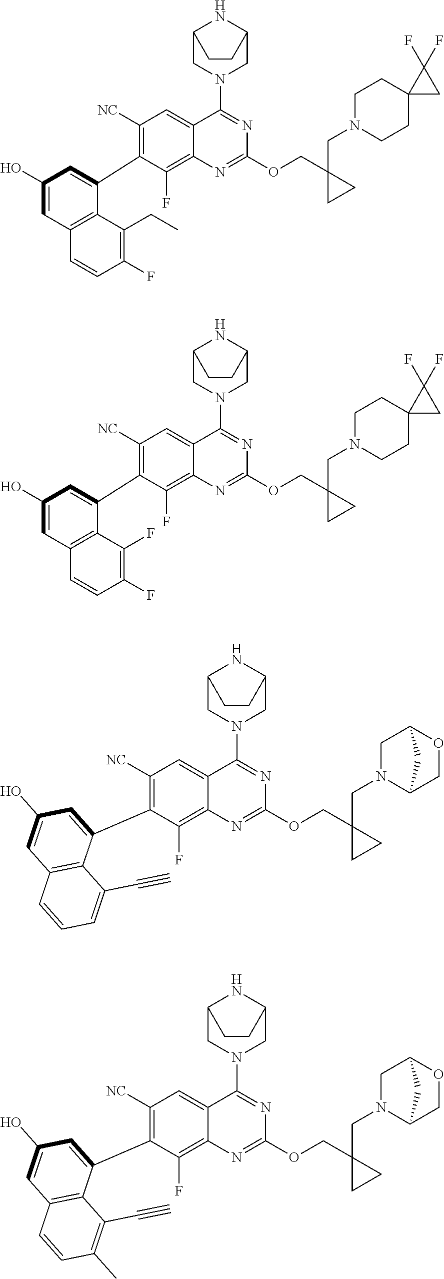

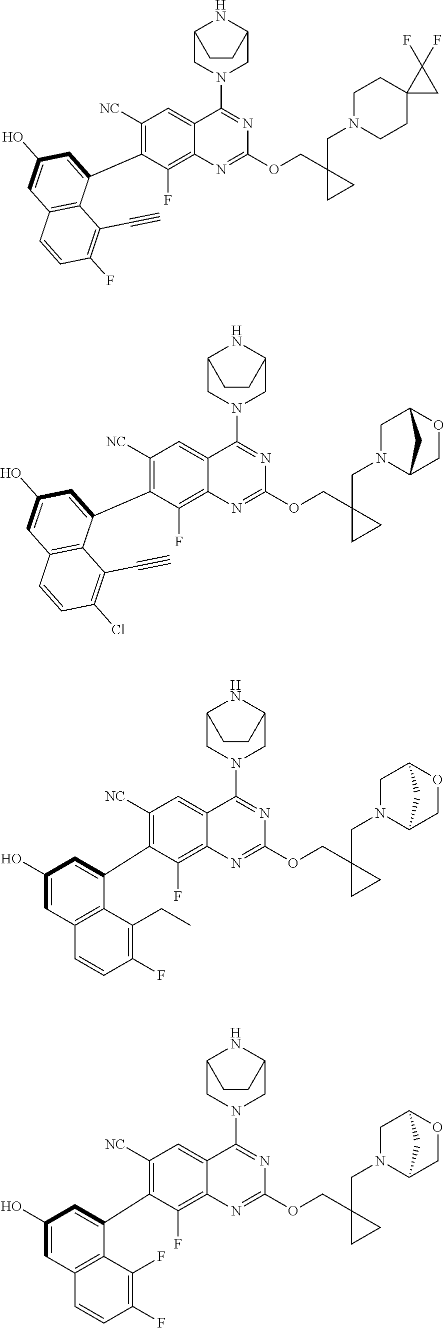

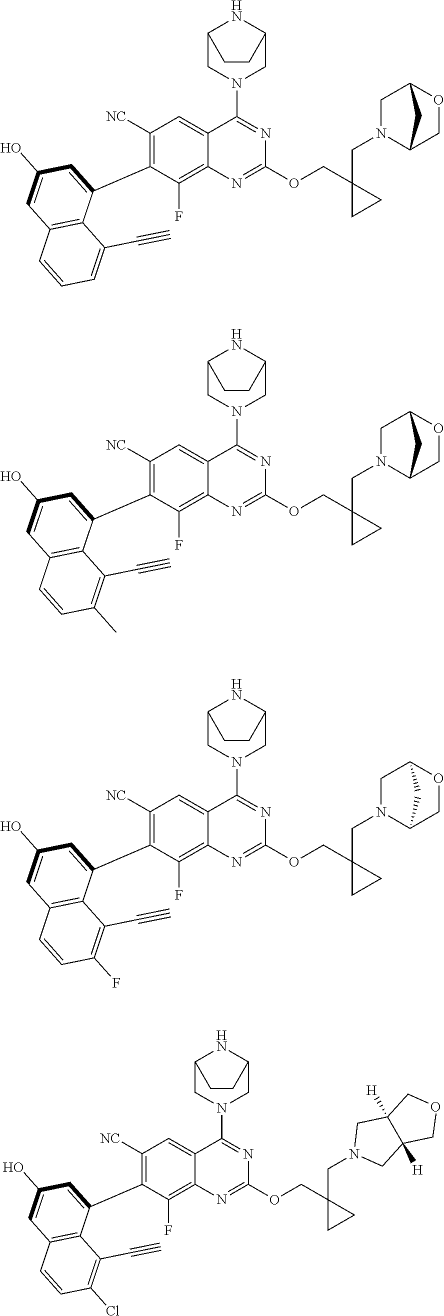

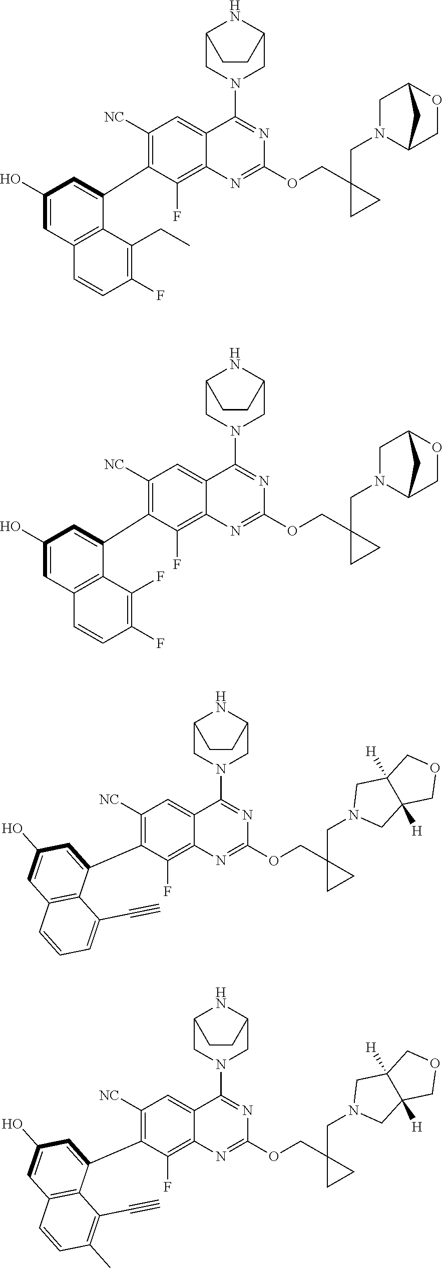

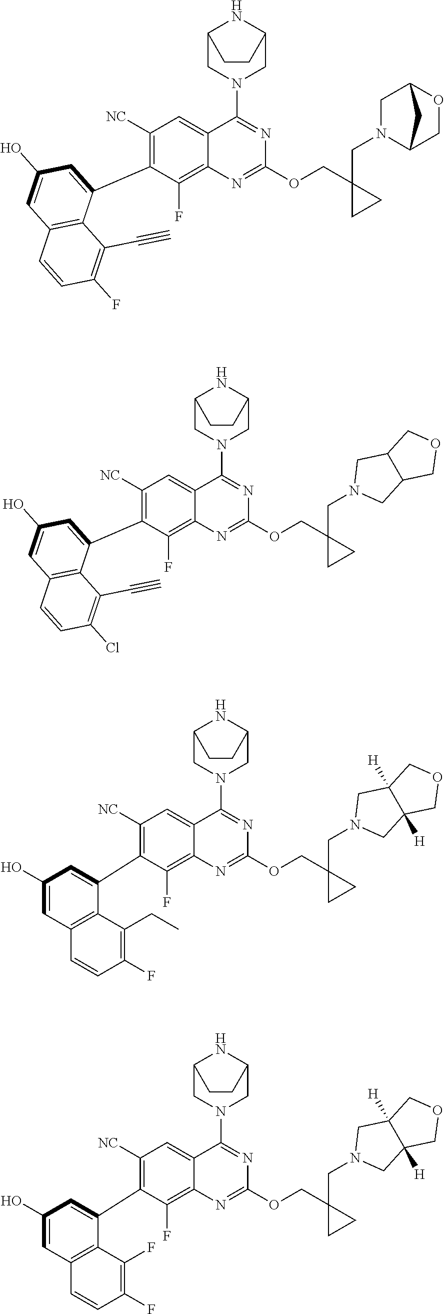

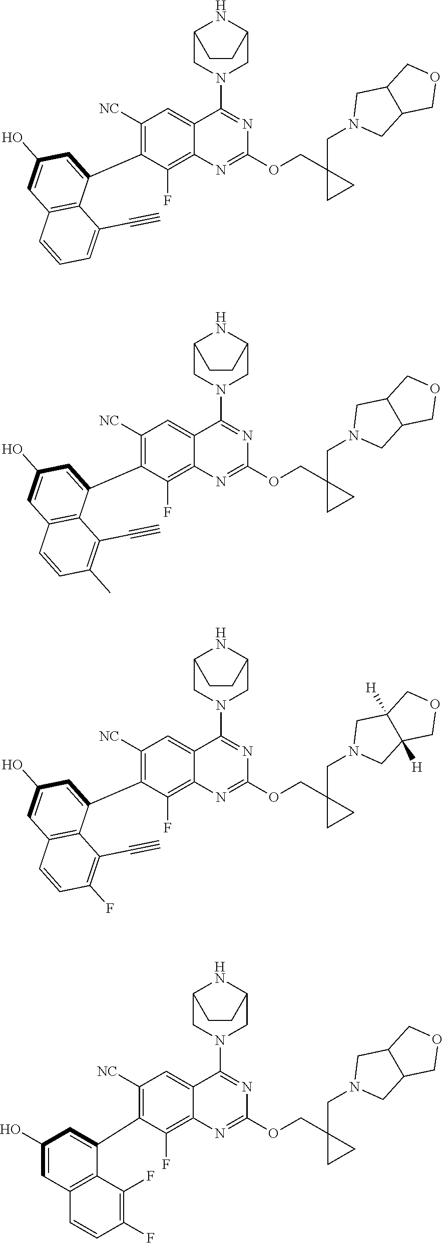

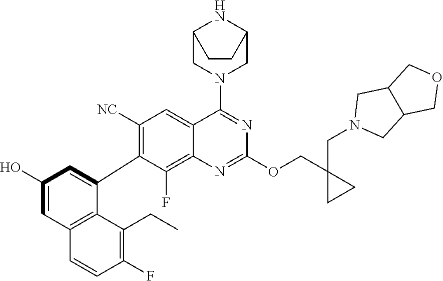

The following compounds could be synthesized with reference to the synthesis procedures of Example 38 and Example 43:

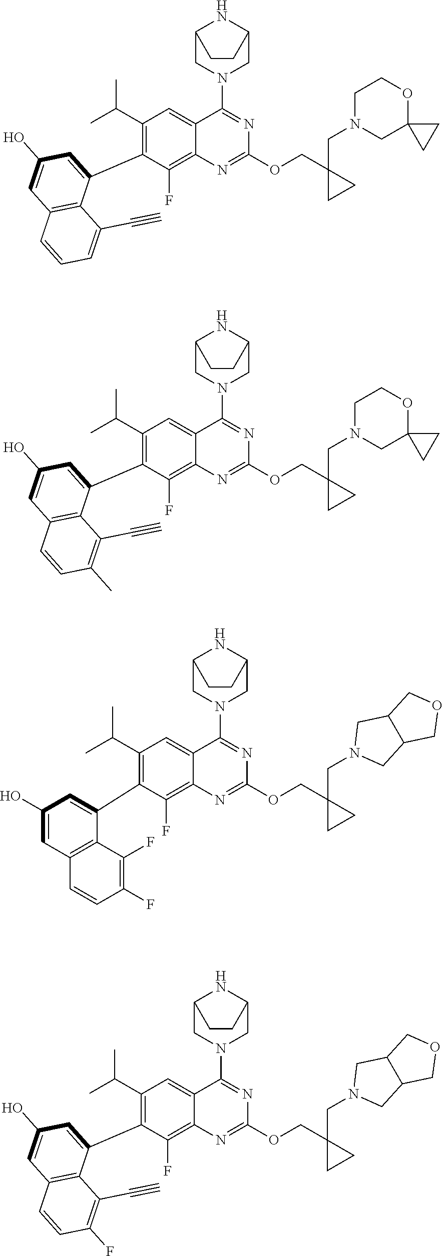

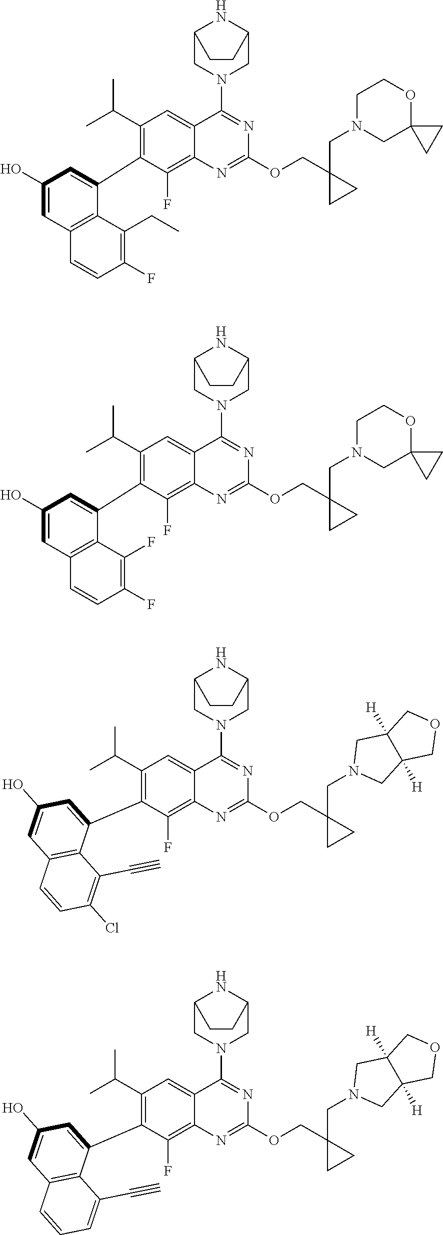

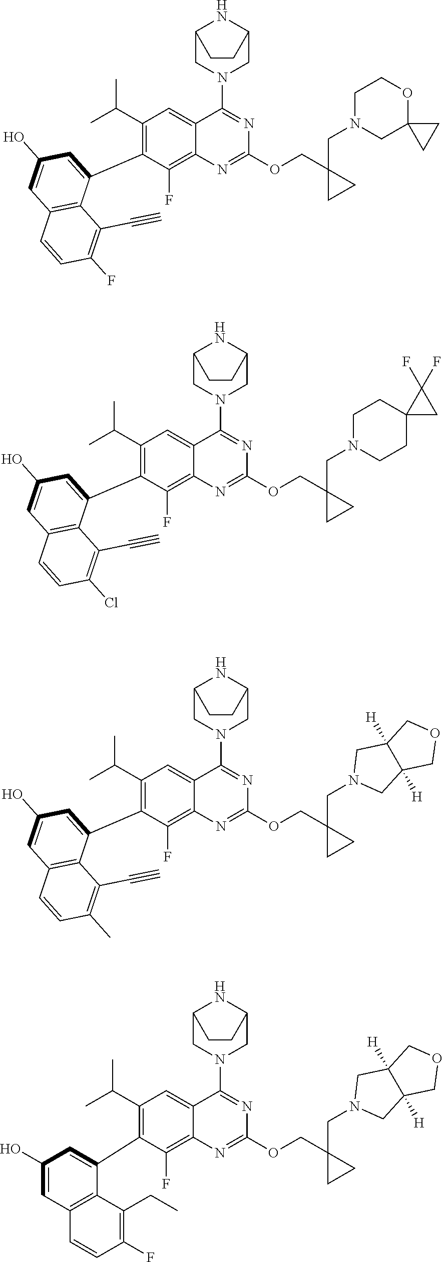

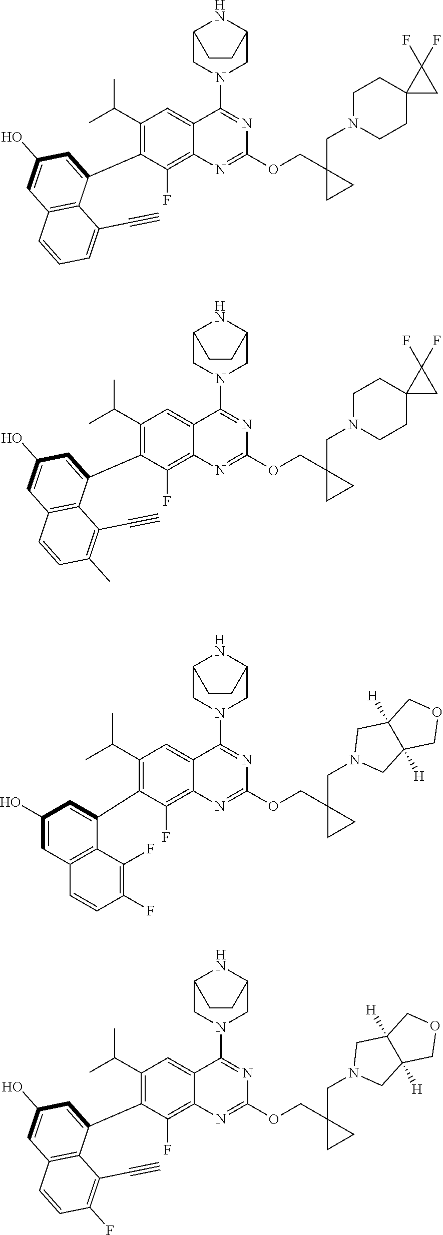

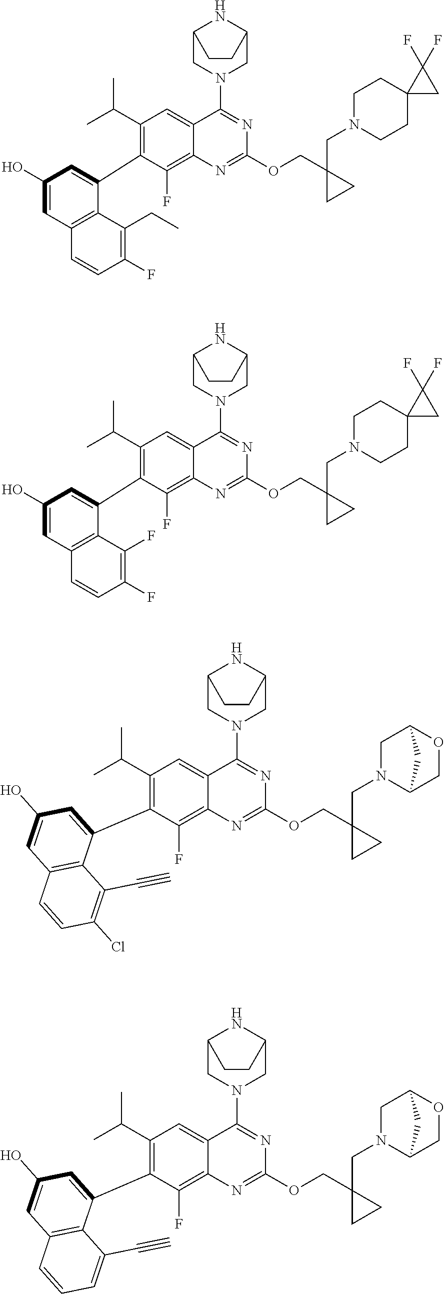

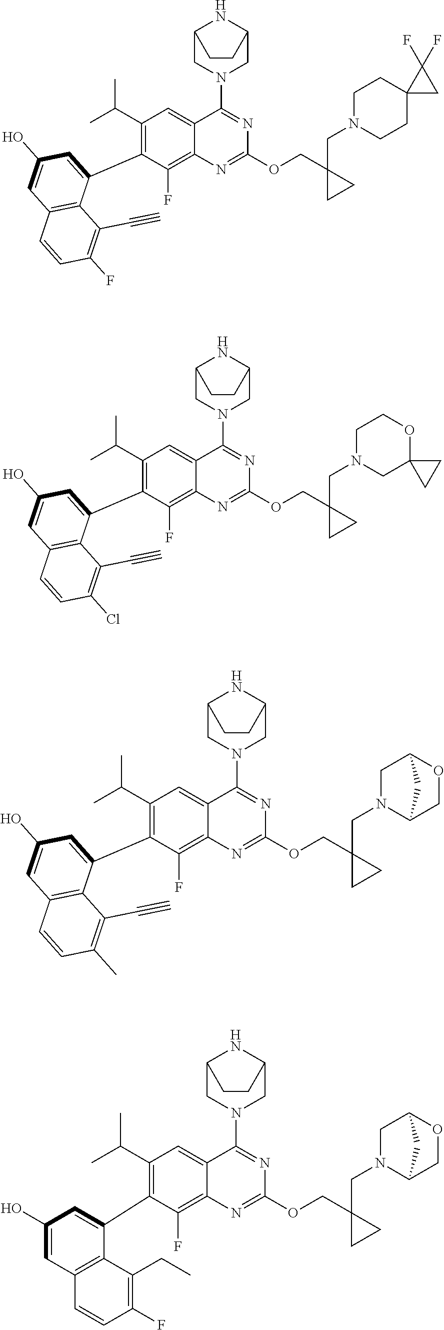

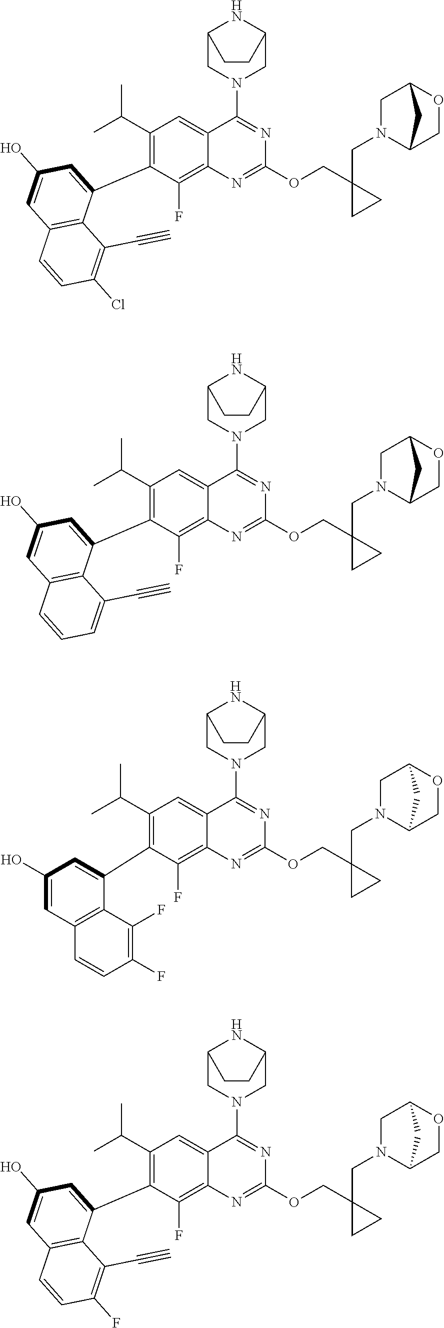

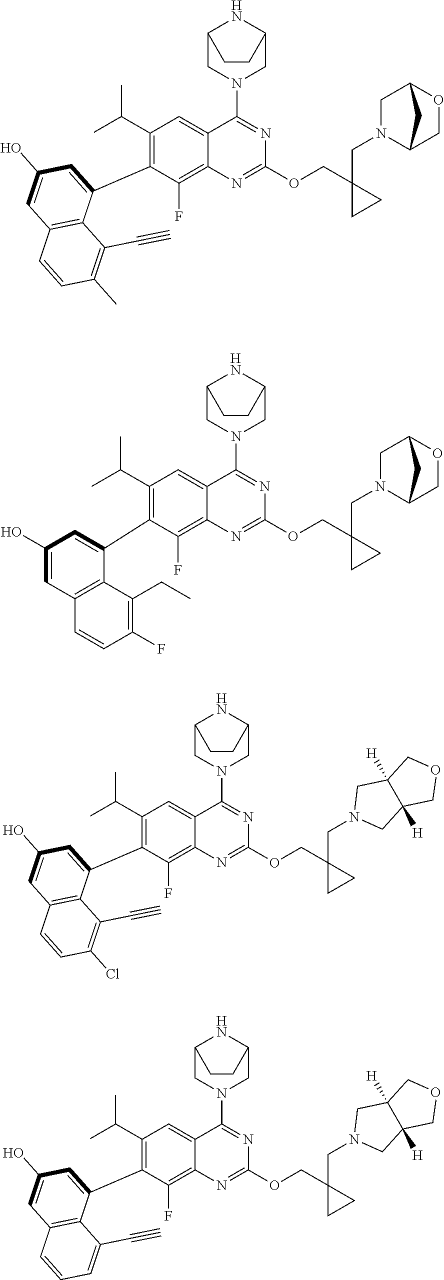

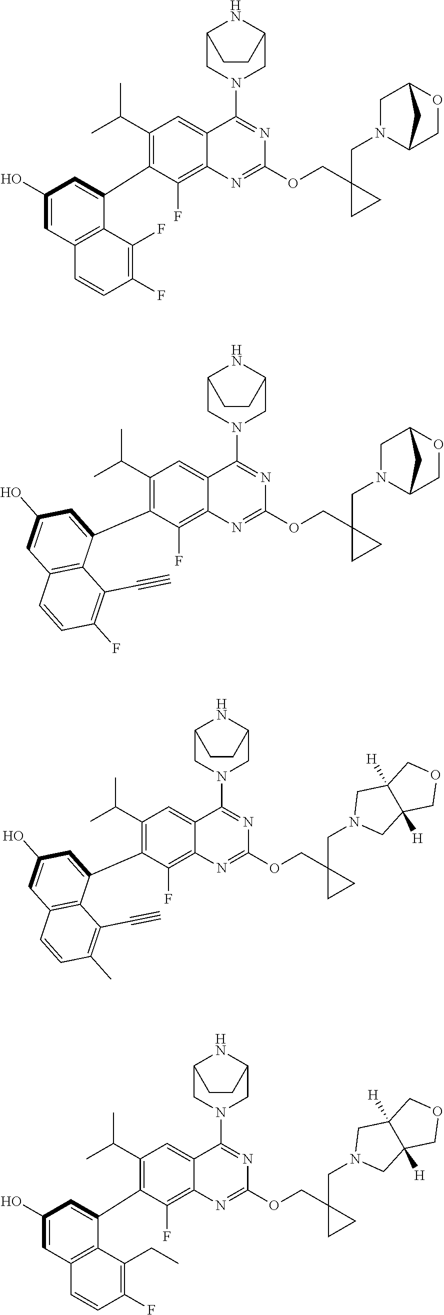

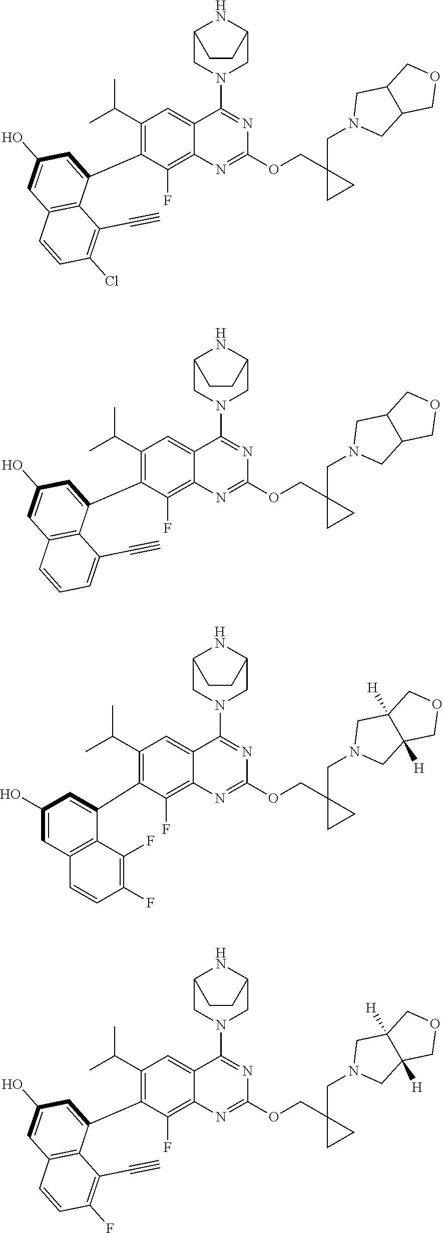

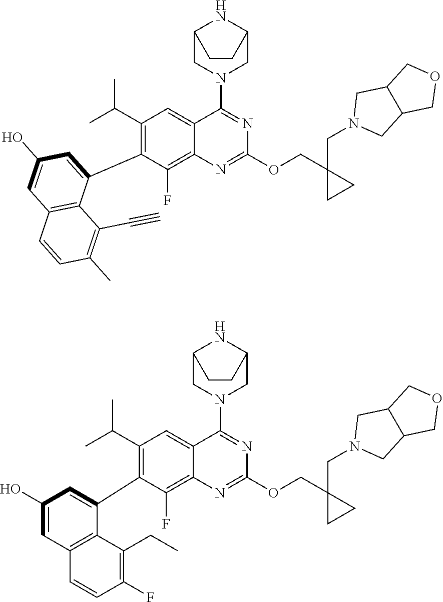

The following compounds could be synthesized with reference to the synthesis procedures of Example 7 and Example 43:

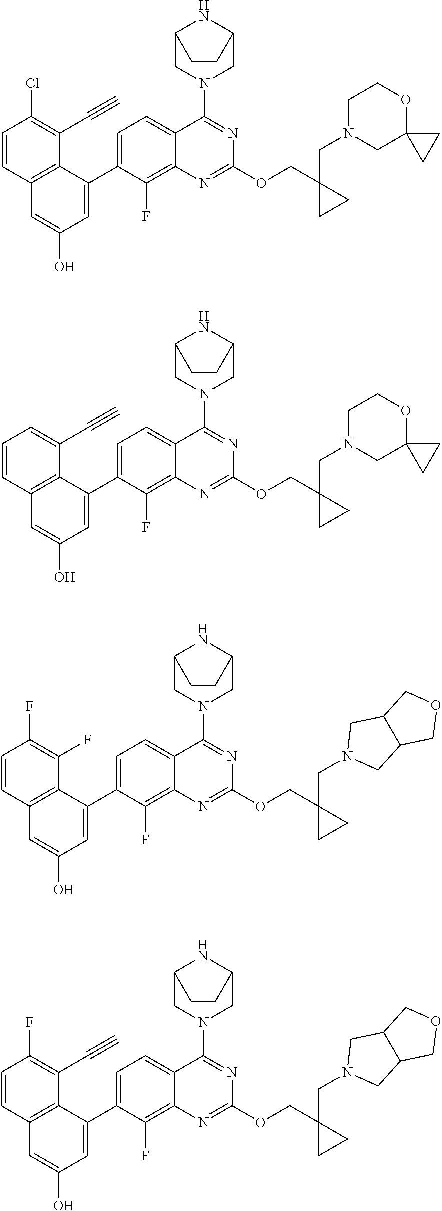 [SMILES: C#Cc1c(Cl)ccc2cc(O)cc(-c3ccc4c(N5CC6CCC(C5)N6)nc(OCC5(CN6CCOC7(CC7)C6)CC5)nc4c3F)c12 ; C#Cc1cccc2cc(O)cc(-c3ccc4c(N5CC6CCC(C5)N6)nc(OCC5(CN6CCOC7(CC7)C6)CC5)nc4c3F)c12 ; Oc1cc(-c2ccc3c(N4CC5CCC(C4)N5)nc(OCC4(CN5CC6COCC6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7COCC7C6)CC5)nc4c3F)c12]

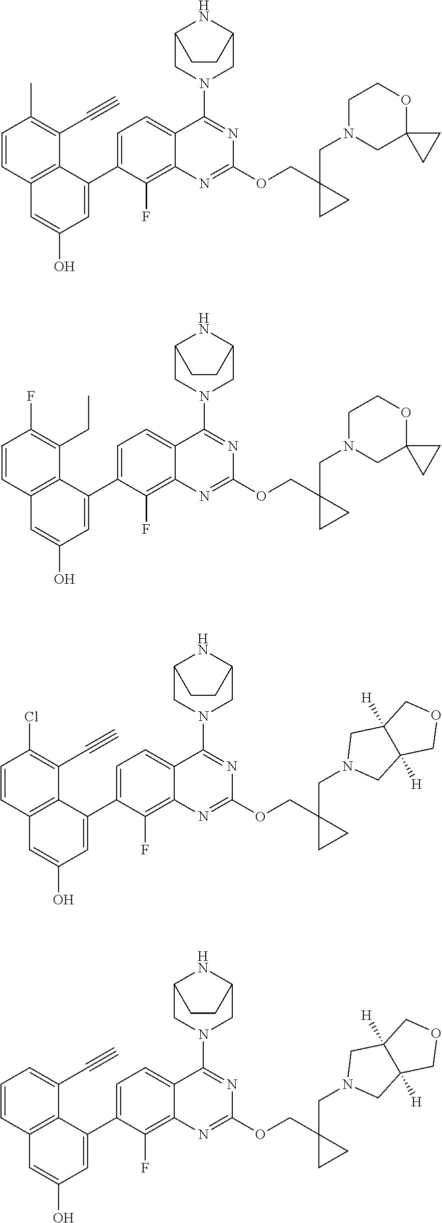 [SMILES: C#Cc1c(C)ccc2cc(O)cc(-c3ccc4c(N5CC6CCC(C5)N6)nc(OCC5(CN6CCOC7(CC7)C6)CC5)nc4c3F)c12 ; CCc1c(F)ccc2cc(O)cc(-c3ccc4c(N5CC6CCC(C5)N6)nc(OCC5(CN6CCOC7(CC7)C6)CC5)nc4c3F)c12 ; C#Cc1c(Cl)ccc2cc(O)cc(-c3ccc4c(N5CC6CCC(C5)N6)nc(OCC5(CN6C[C@H]7COC[C@H]7C6)CC5)nc4c3F)c12 ; C#Cc1cccc2cc(O)cc(-c3ccc4c(N5CC6CCC(C5)N6)nc(OCC5(CN6C[C@H]7COC[C@H]7C6)CC5)nc4c3F)c12]

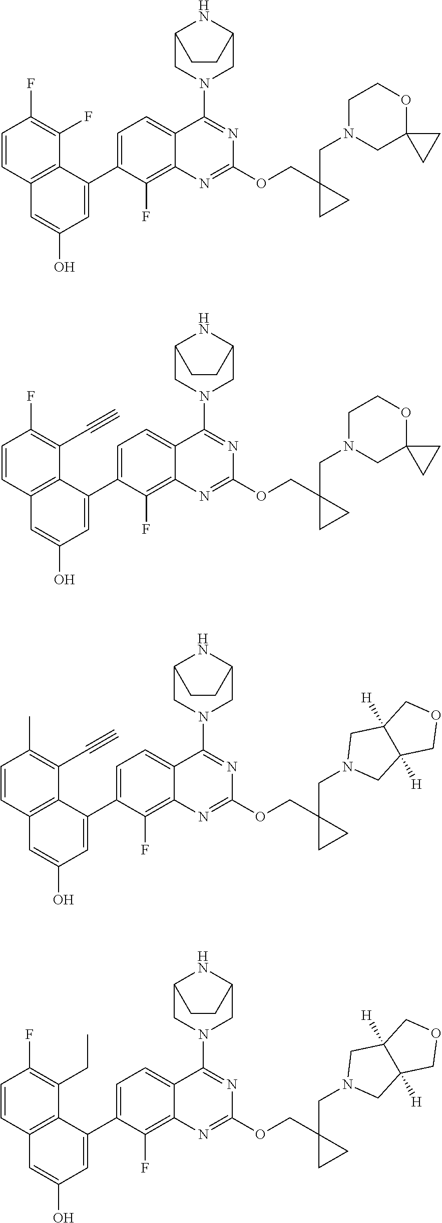 [SMILES: Oc1cc(-c2ccc3c(N4CC5CCC(C4)N5)nc(OCC4(CN5CCOC6(CC6)C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CCOC7(CC7)C6)CC5)nc4c3F)c12 ; C#Cc1c(C)ccc2cc(O)cc(-c3ccc4c(N5CC6CCC(C5)N6)nc(OCC5(CN6C[C@H]7COC[C@H]7C6)CC5)nc4c3F)c12 ; CCc1c(F)ccc2cc(O)cc(-c3ccc4c(N5CC6CCC(C5)N6)nc(OCC5(CN6C[C@H]7COC[C@H]7C6)CC5)nc4c3F)c12]

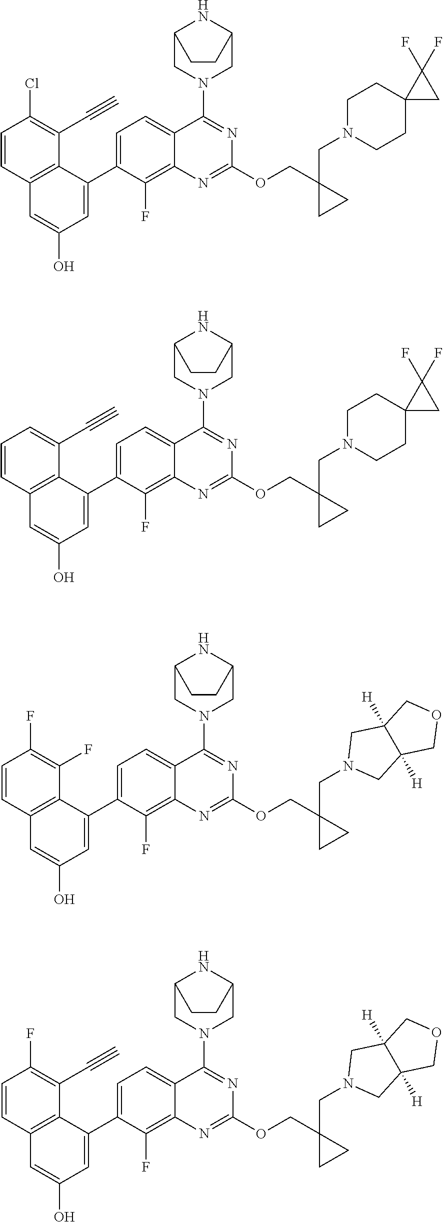 [SMILES: C#Cc1c(Cl)ccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; Oc1cc(-c2ccc3c(N4CC5CCC(C4)N5)nc(OCC4(CN5C[C@H]6COC[C@H]6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7COC[C@H]7C6)CC5)nc4c3F)c12]

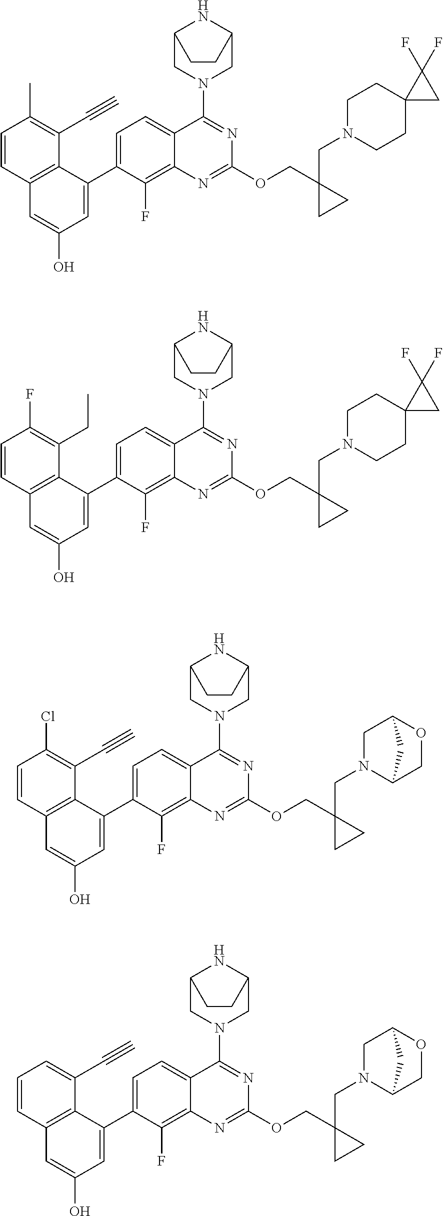 [SMILES: C#Cc1c(C)ccc2cc(O)cc(-c3ccc4c(N5CC6CCC(C5)N6)nc(OCC5(CN6CCC7(CC6)CC7(F)F)CC5)nc4c3F)c12 ; CCc1c(F)ccc2cc(O)cc(-c3ccc4c(N5CC6CCC(C5)N6)nc(OCC5(CN6CCC7(CC6)CC7(F)F)CC5)nc4c3F)c12 ; C#Cc1c(Cl)ccc2cc(O)cc(-c3ccc4c(N5CC6CCC(C5)N6)nc(OCC5(CN6C[C@@H]7C[C@H]6CO7)CC5)nc4c3F)c12 ; C#Cc1cccc2cc(O)cc(-c3ccc4c(N5CC6CCC(C5)N6)nc(OCC5(CN6C[C@@H]7C[C@H]6CO7)CC5)nc4c3F)c12]

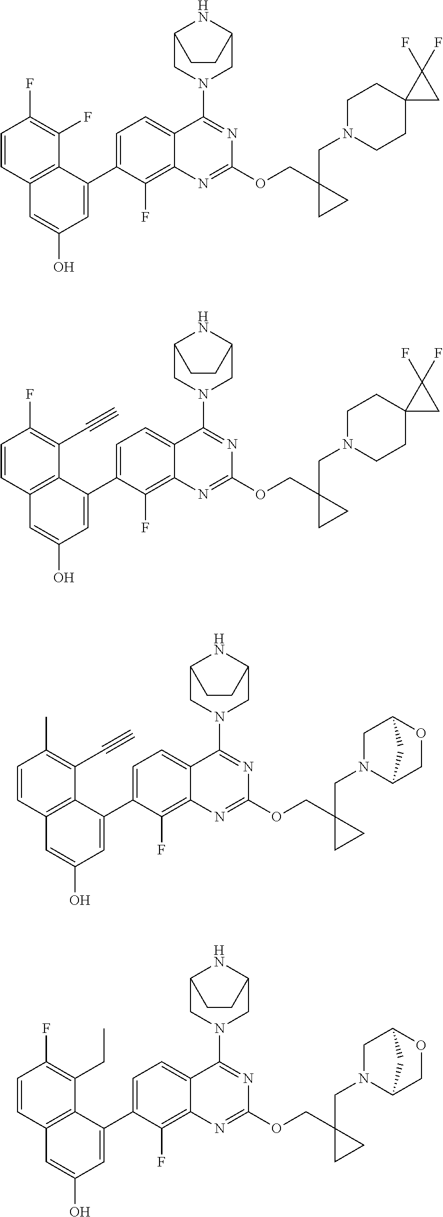 [SMILES: Oc1cc(-c2ccc3c(N4CC5CCC(C4)N5)nc(OCC4(CN5CCC6(CC5)CC6(F)F)CC4)nc3c2F)c2c(F)c(F)ccc2c1 ; C#Cc1c(F)ccc2cc(O)cc(-c3ccc4c(N5CC6CCC(C5)N6)nc(OCC5(CN6CCC7(CC6)CC7(F)F)CC5)nc4c3F)c12 ; C#Cc1c(C)ccc2cc(O)cc(-c3ccc4c(N5CC6CCC(C5)N6)nc(OCC5(CN6C[C@@H]7C[C@H]6CO7)CC5)nc4c3F)c12 ; CCc1c(F)ccc2cc(O)cc(-c3ccc4c(N5CC6CCC(C5)N6)nc(OCC5(CN6C[C@@H]7C[C@H]6CO7)CC5)nc4c3F)c12]

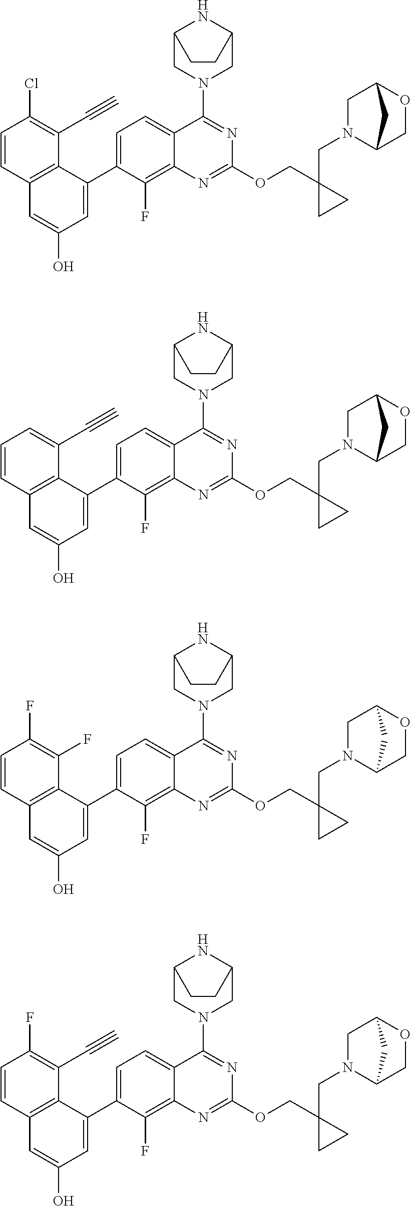 [SMILES: C#Cc1c(Cl)ccc2cc(O)cc(-c3ccc4c(N5CC6CCC(C5)N6)nc(OCC5(CN6C[C@H]7C[C@@H]6CO7)CC5)nc4c3F)c12 ; C#Cc1cccc2cc(O)cc(-c3ccc4c(N5CC6CCC(C5)N6)nc(OCC5(CN6C[C@H]7C[C@@H]6CO7)CC5)nc4c3F)c12 ; Oc1cc(-c2ccc3c(N4CC5CCC(C4)N5)nc(OCC4(CN5C[C@@H]6C[C@H]5CO6)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@@H]7C[C@H]6CO7)CC5)nc4c3F)c12]

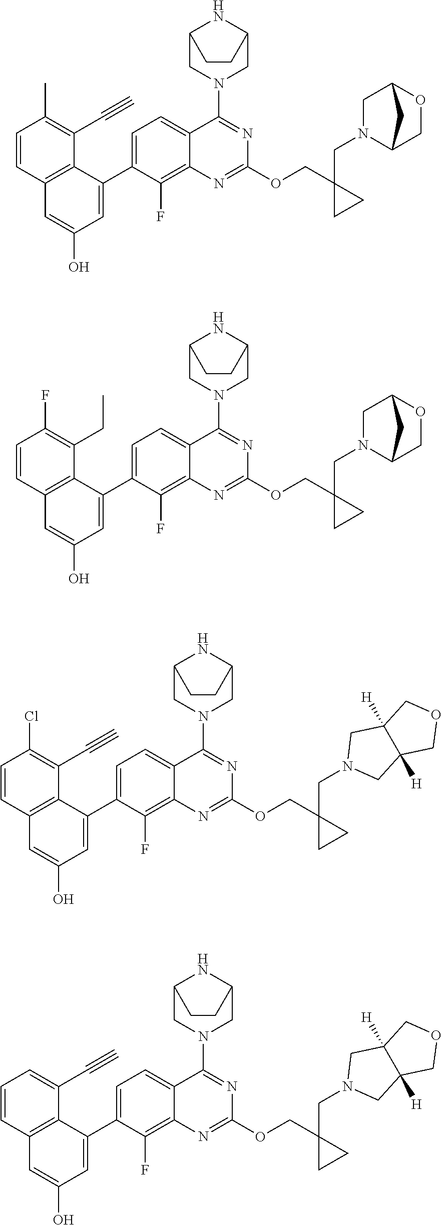 [SMILES: C#Cc1c(C)ccc2cc(O)cc(-c3ccc4c(N5CC6CCC(C5)N6)nc(OCC5(CN6C[C@H]7C[C@@H]6CO7)CC5)nc4c3F)c12 ; CCc1c(F)ccc2cc(O)cc(-c3ccc4c(N5CC6CCC(C5)N6)nc(OCC5(CN6C[C@H]7C[C@@H]6CO7)CC5)nc4c3F)c12 ; C#Cc1c(Cl)ccc2cc(O)cc(-c3ccc4c(N5CC6CCC(C5)N6)nc(OCC5(CN6C[C@H]7COC[C@@H]7C6)CC5)nc4c3F)c12 ; C#Cc1cccc2cc(O)cc(-c3ccc4c(N5CC6CCC(C5)N6)nc(OCC5(CN6C[C@H]7COC[C@@H]7C6)CC5)nc4c3F)c12]

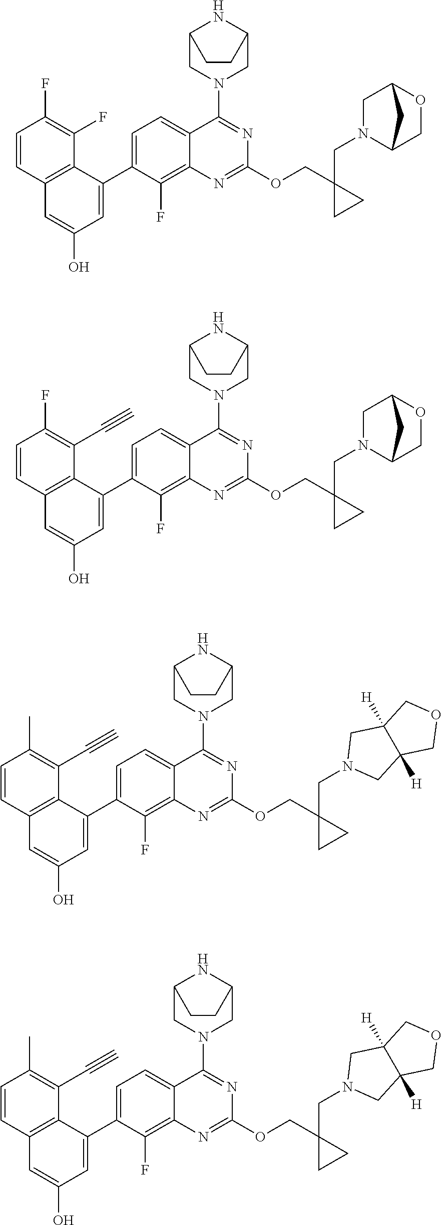 [SMILES: Oc1cc(-c2ccc3c(N4CC5CCC(C4)N5)nc(OCC4(CN5C[C@H]6C[C@@H]5CO6)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7C[C@@H]6CO7)CC5)nc4c3F)c12 ; C#Cc1c(C)ccc2cc(O)cc(-c3ccc4c(N5CC6CCC(C5)N6)nc(OCC5(CN6C[C@H]7COC[C@@H]7C6)CC5)nc4c3F)c12 ; C#Cc1c(C)ccc2cc(O)cc(-c3ccc4c(N5CC6CCC(C5)N6)nc(OCC5(CN6C[C@H]7COC[C@@H]7C6)CC5)nc4c3F)c12]

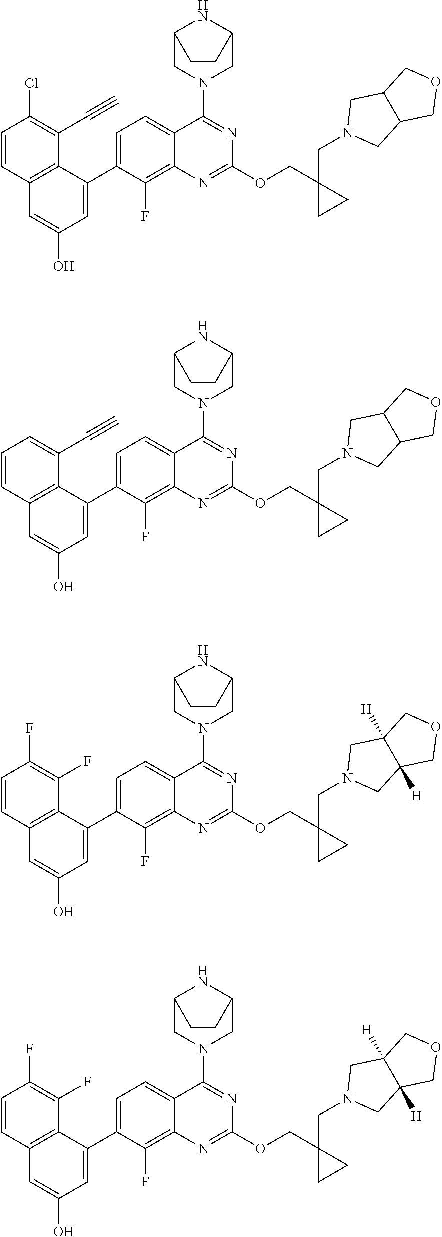 [SMILES: C#Cc1c(Cl)ccc2cc(O)cc(-c3ccc4c(N5CC6CCC(C5)N6)nc(OCC5(CN6CC7COCC7C6)CC5)nc4c3F)c12 ; C#Cc1cccc2cc(O)cc(-c3ccc4c(N5CC6CCC(C5)N6)nc(OCC5(CN6CC7COCC7C6)CC5)nc4c3F)c12 ; Oc1cc(-c2ccc3c(N4CC5CCC(C4)N5)nc(OCC4(CN5C[C@H]6COC[C@@H]6C5)CC4)nc3c2F)c2c(F)c(F)ccc2c1 ; Oc1cc(-c2ccc3c(N4CC5CCC(C4)N5)nc(OCC4(CN5C[C@H]6COC[C@@H]6C5)CC4)nc3c2F)c2c(F)c(F)ccc2c1]

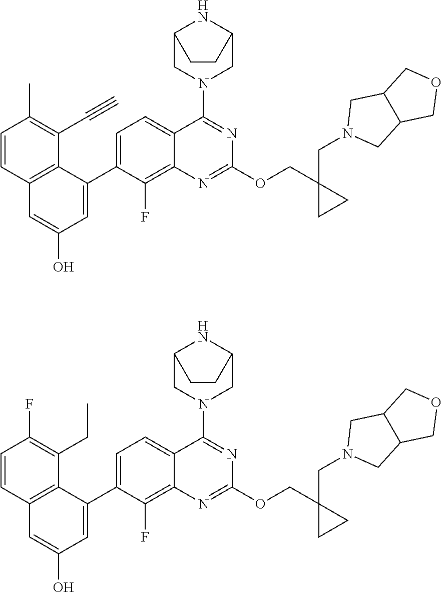 [SMILES: C#Cc1c(C)ccc2cc(O)cc(-c3ccc4c(N5CC6CCC(C5)N6)nc(OCC5(CN6CC7COCC7C6)CC5)nc4c3F)c12 ; CCc1c(F)ccc2cc(O)cc(-c3ccc4c(N5CC6CCC(C5)N6)nc(OCC5(CN6CC7COCC7C6)CC5)nc4c3F)c12]

The following compounds could be synthesized with reference to the synthesis procedures of above Examples.

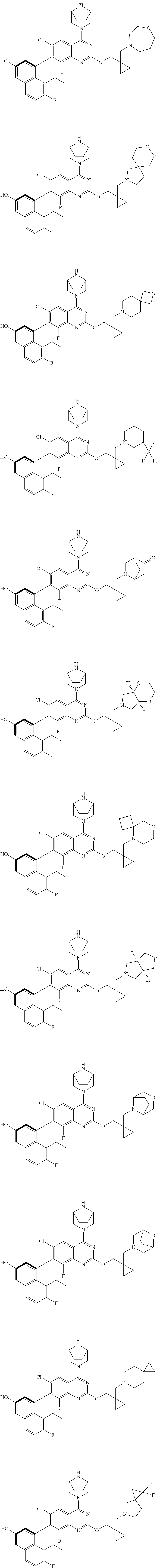

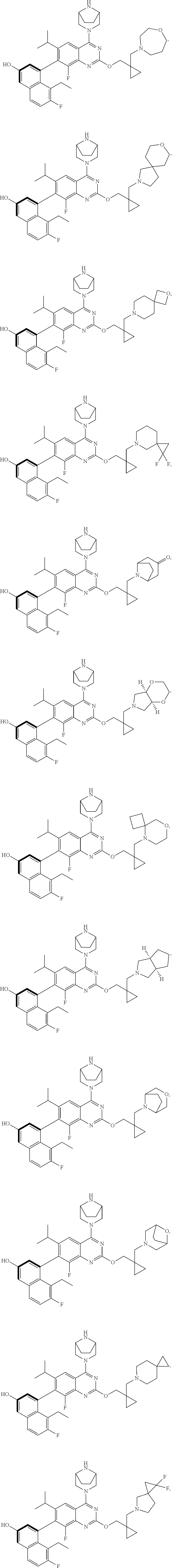

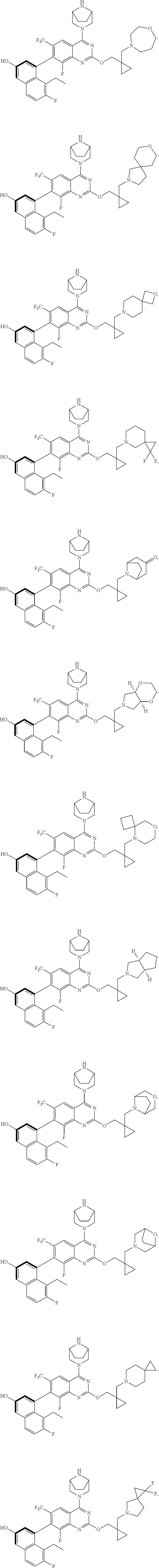

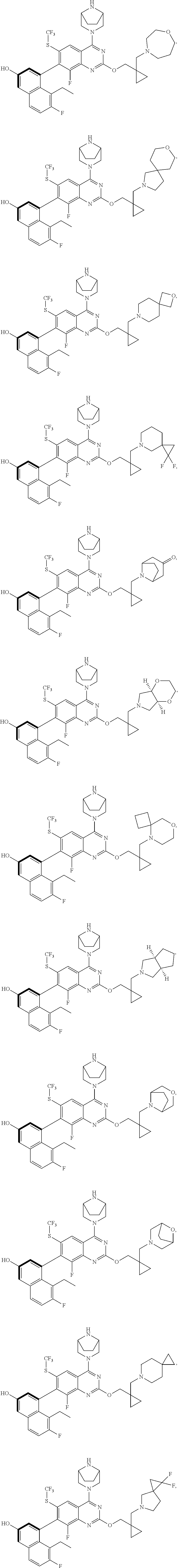

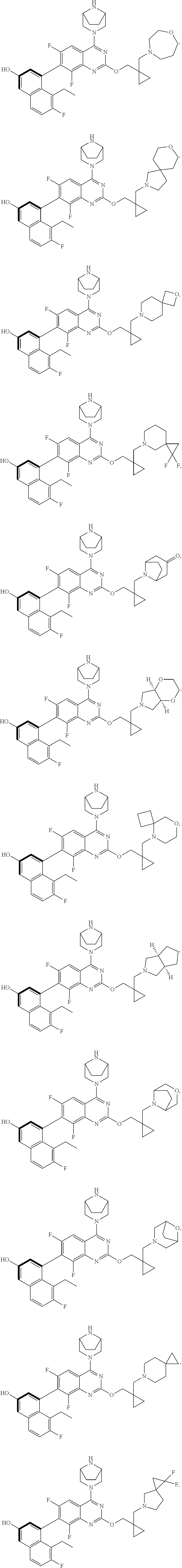

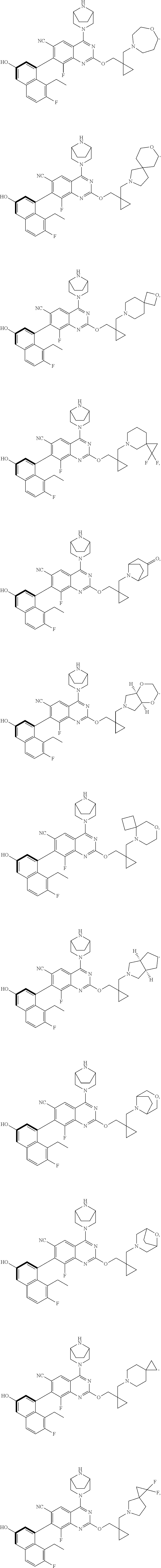

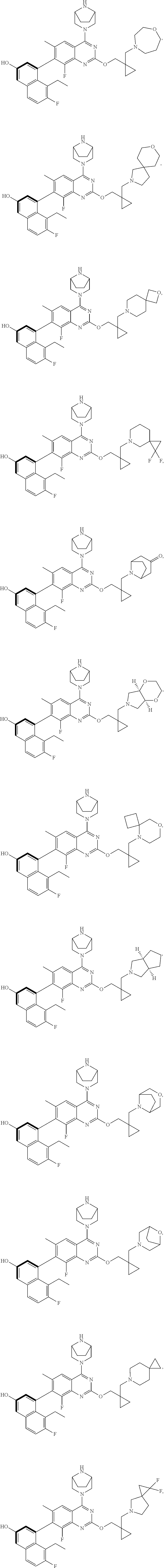

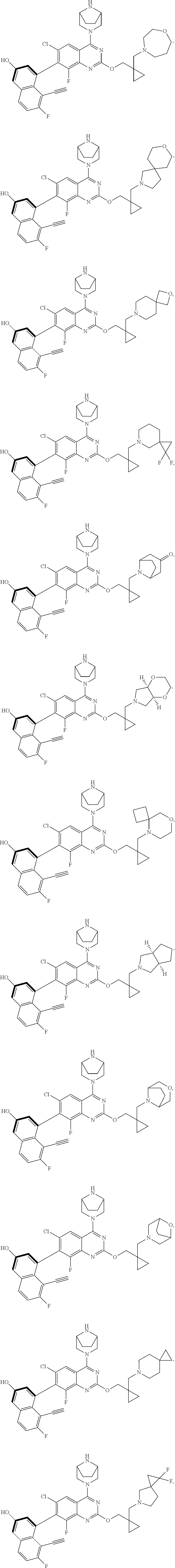

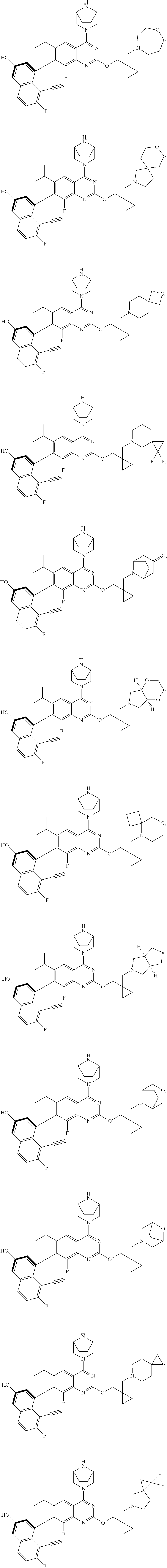

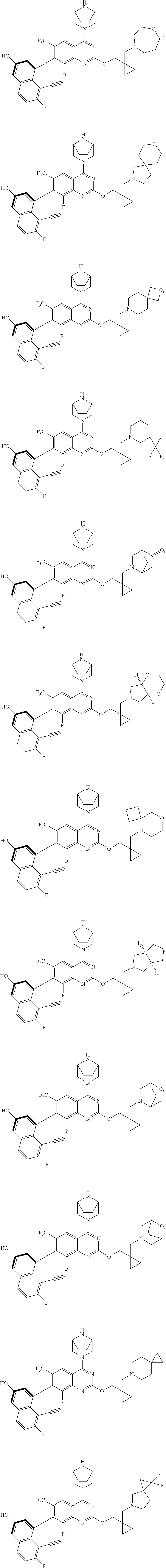

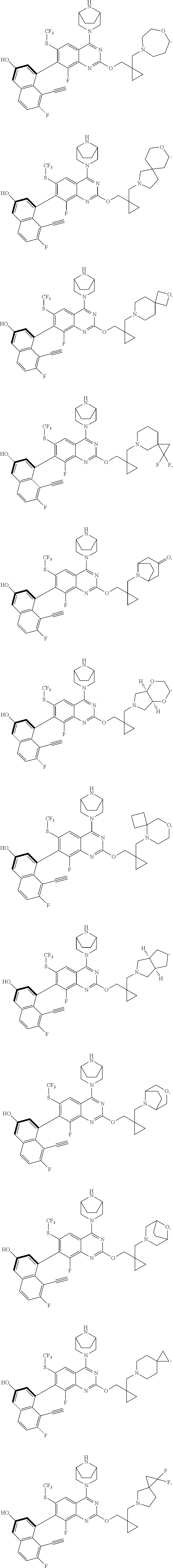

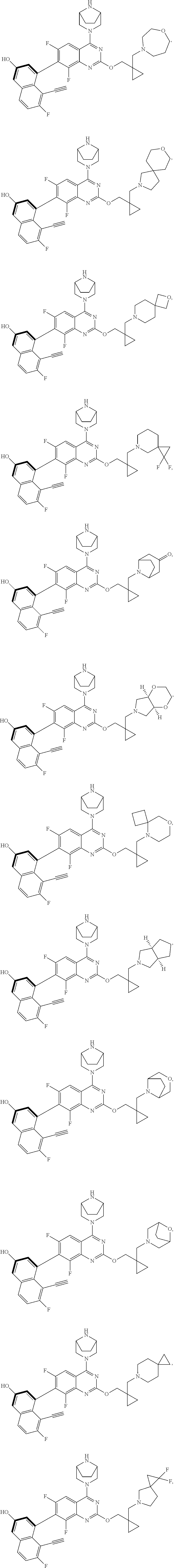

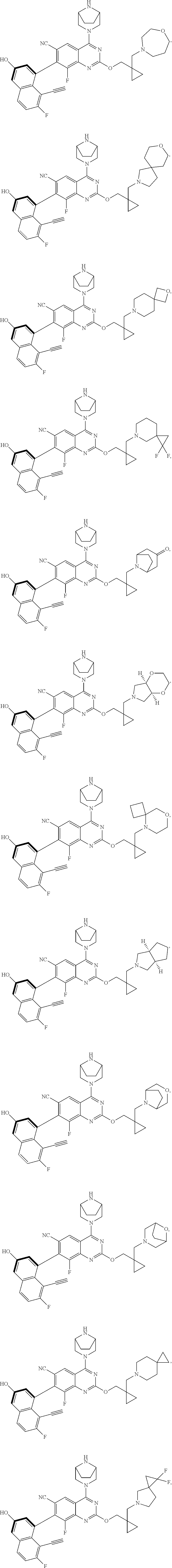

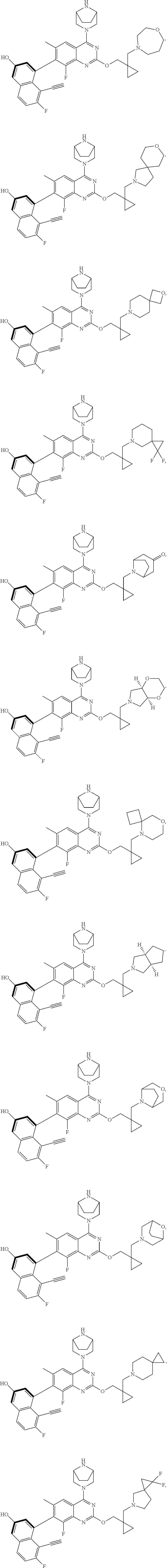

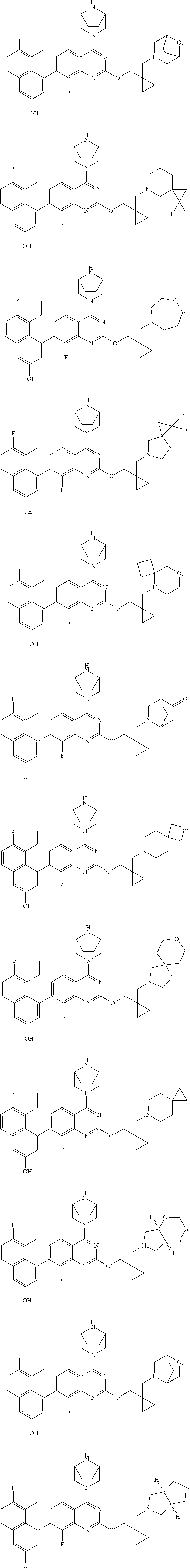

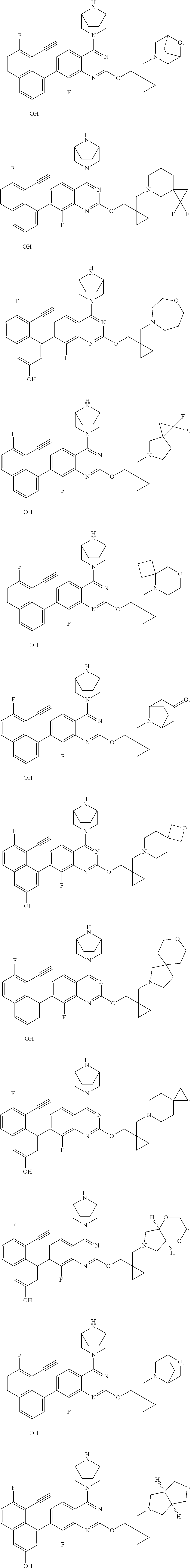

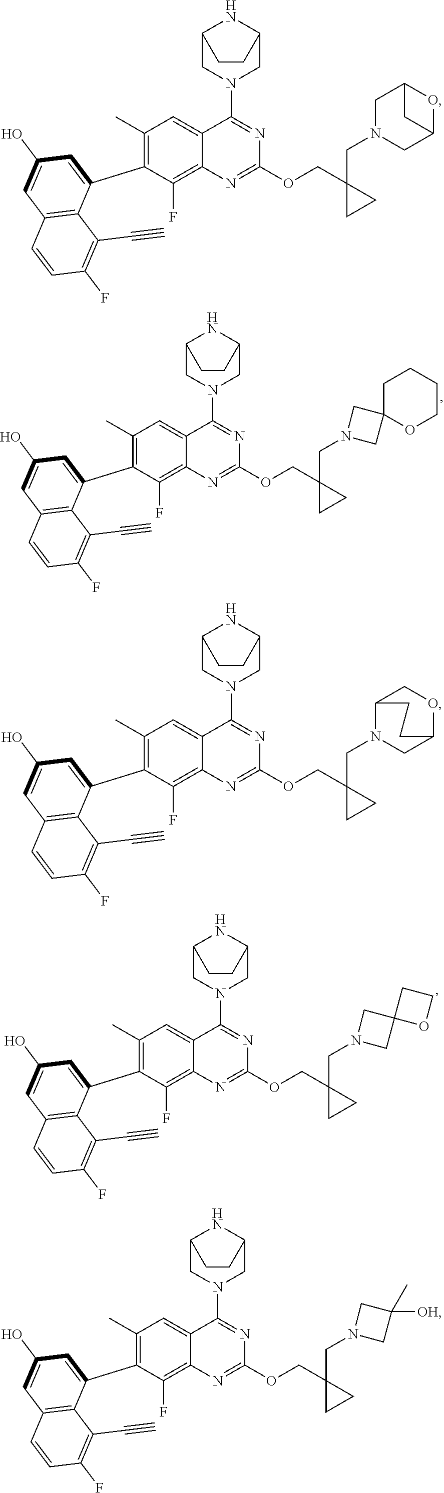

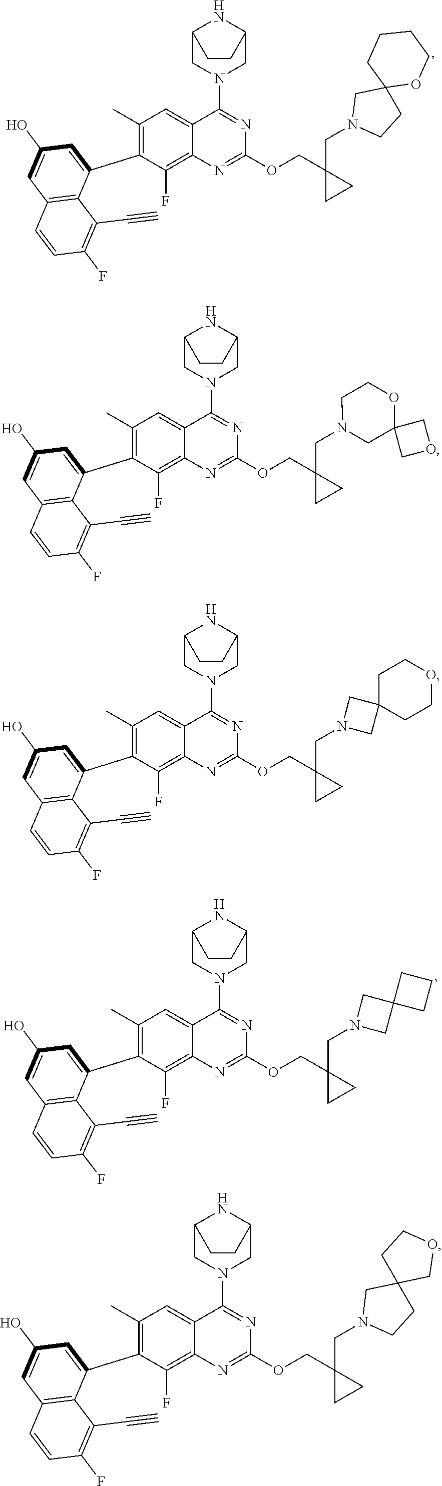

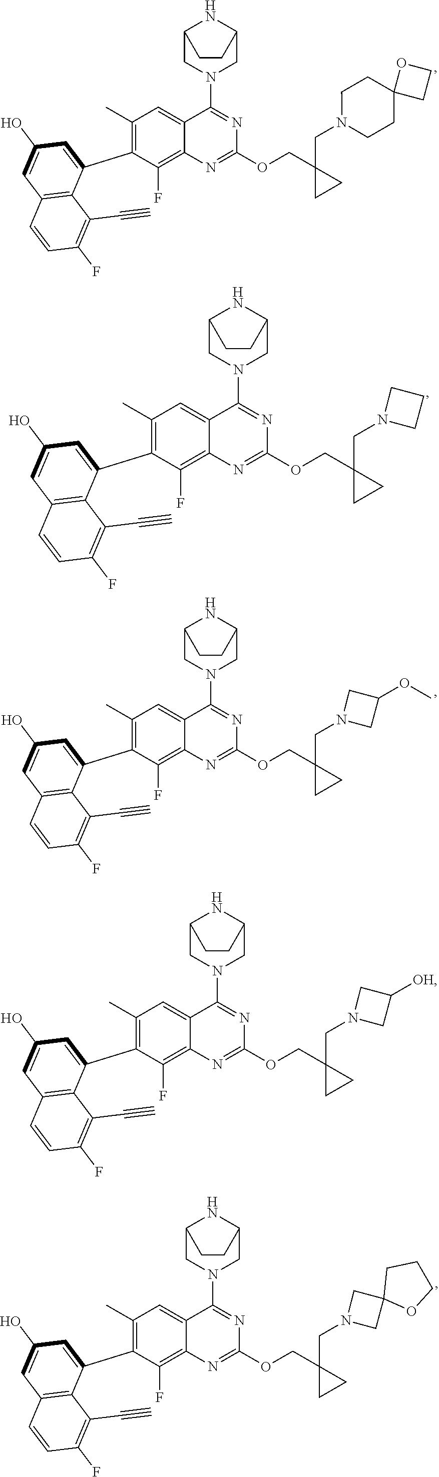

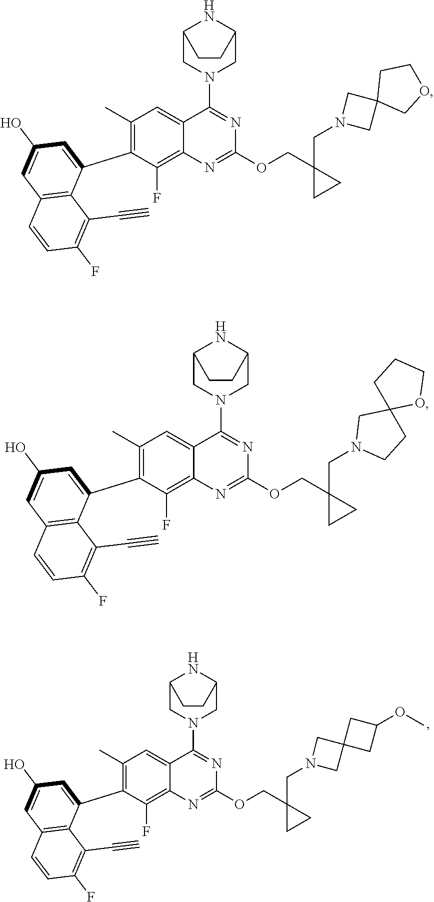

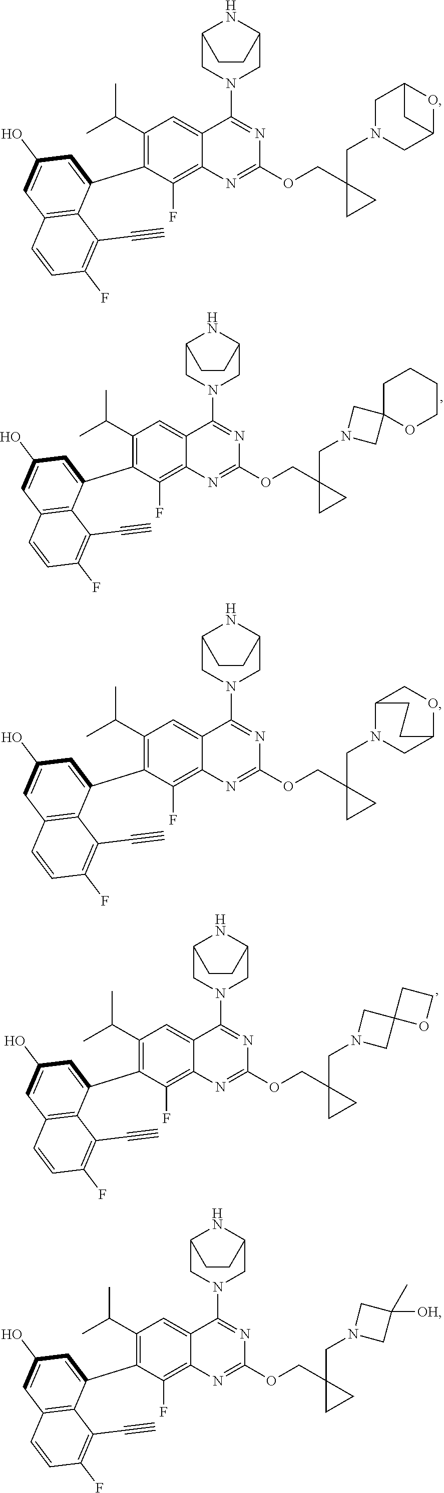

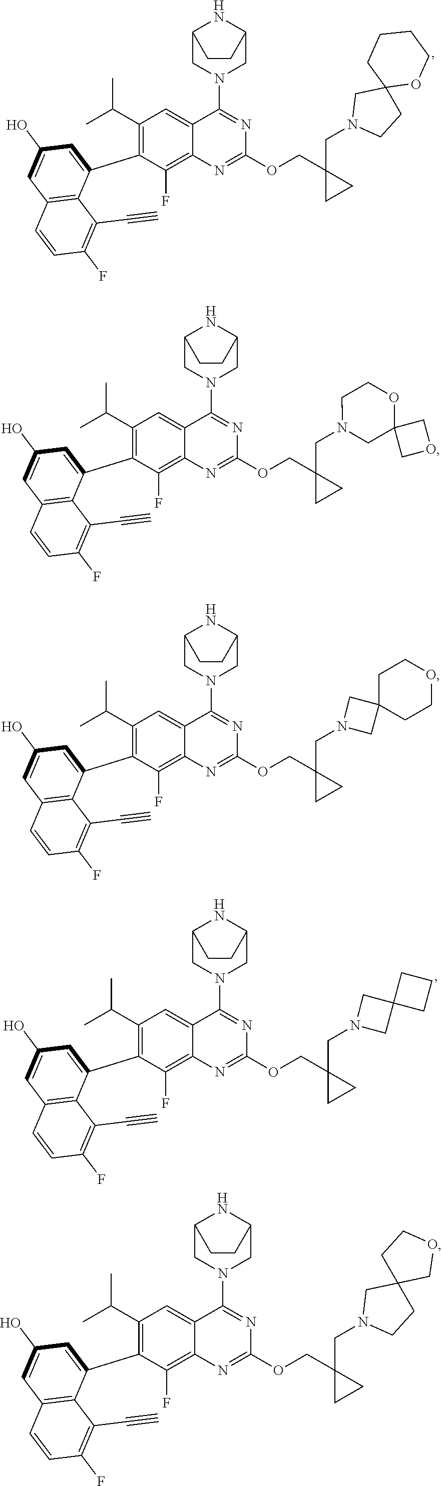

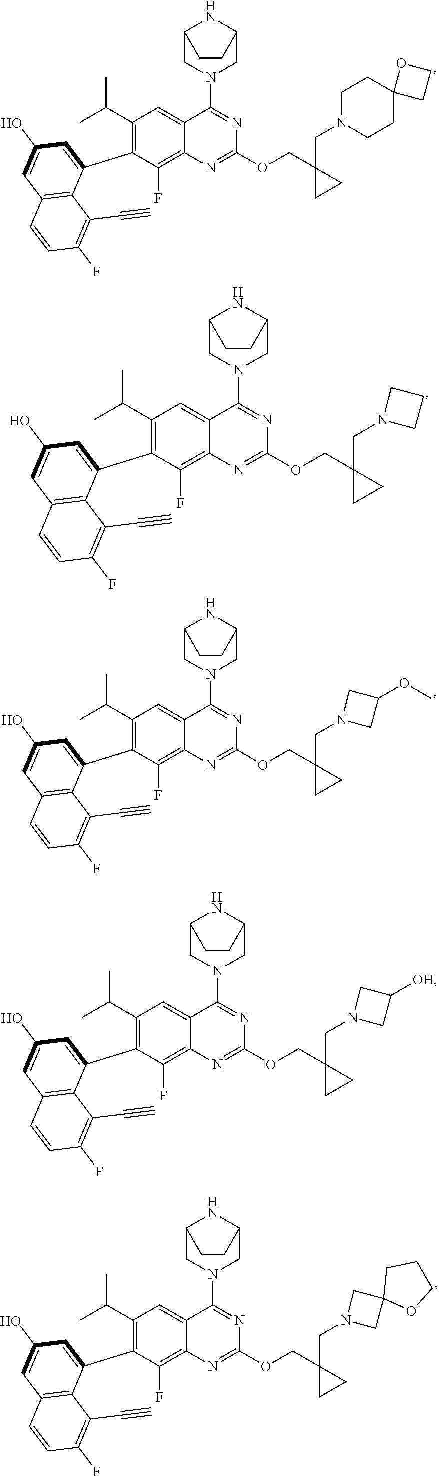

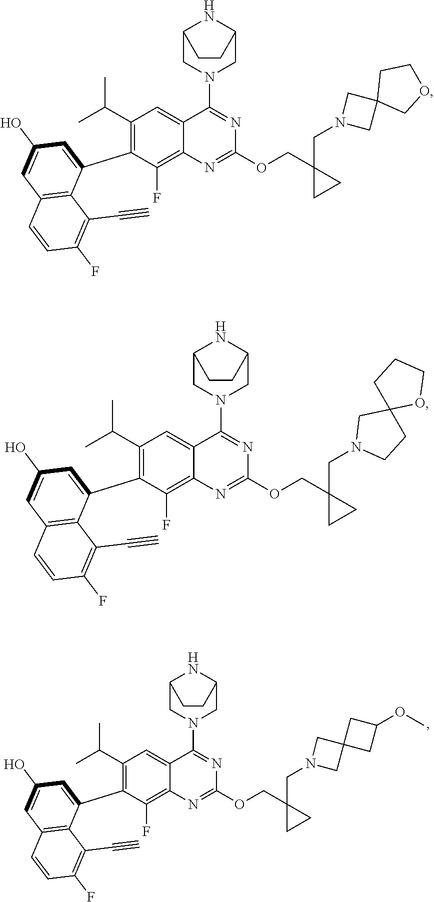

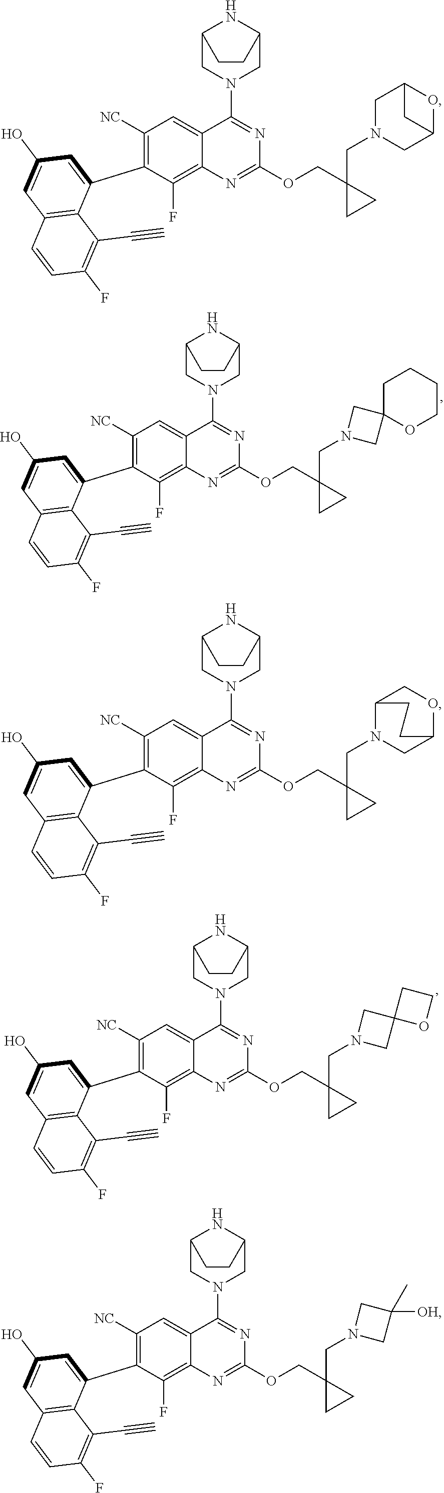

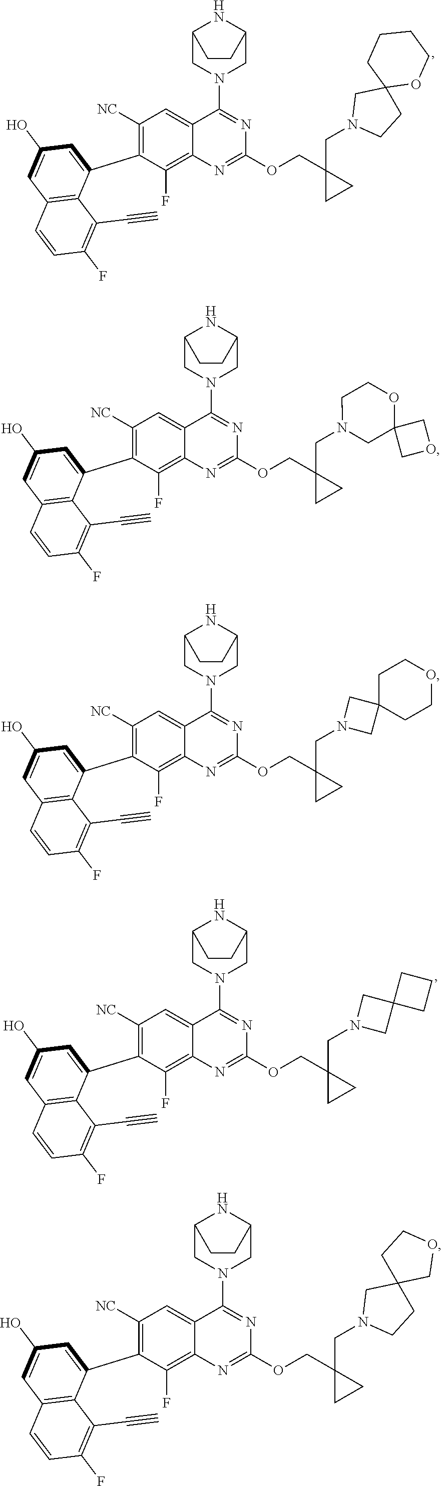

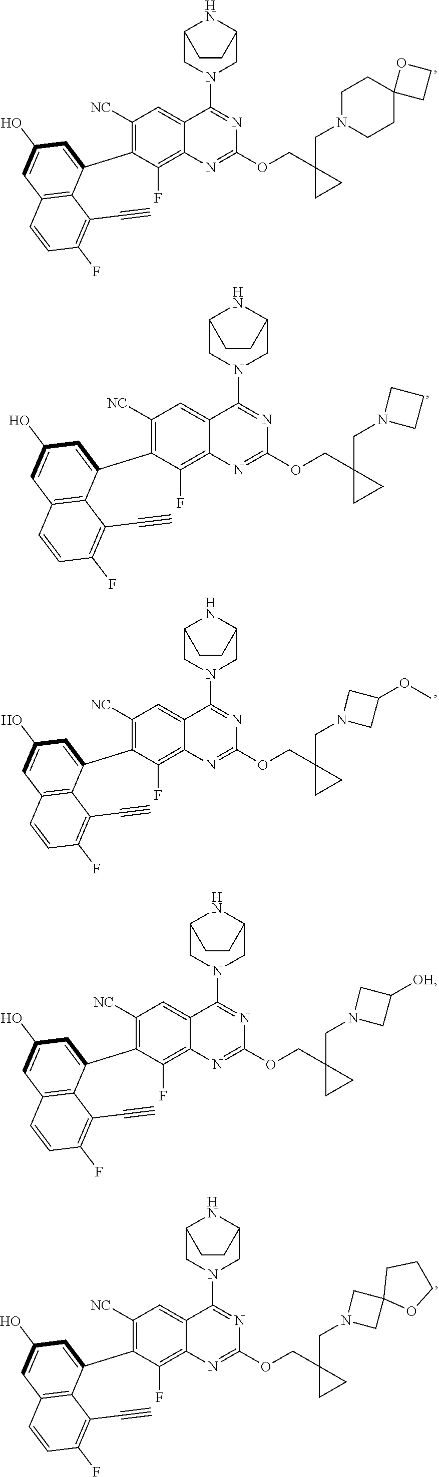

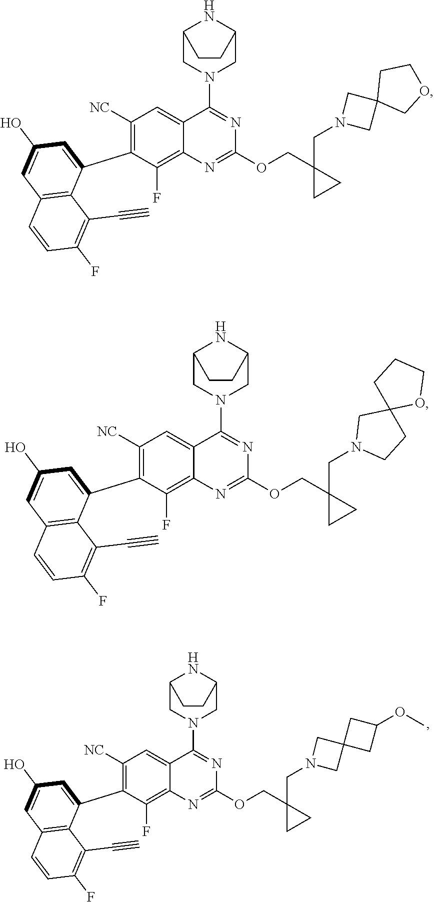

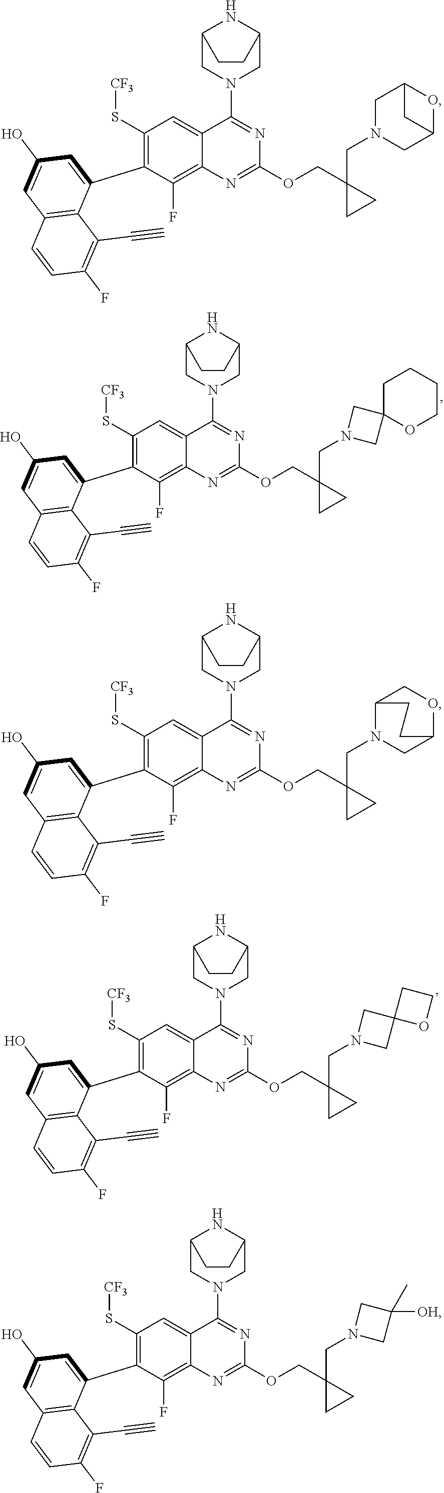

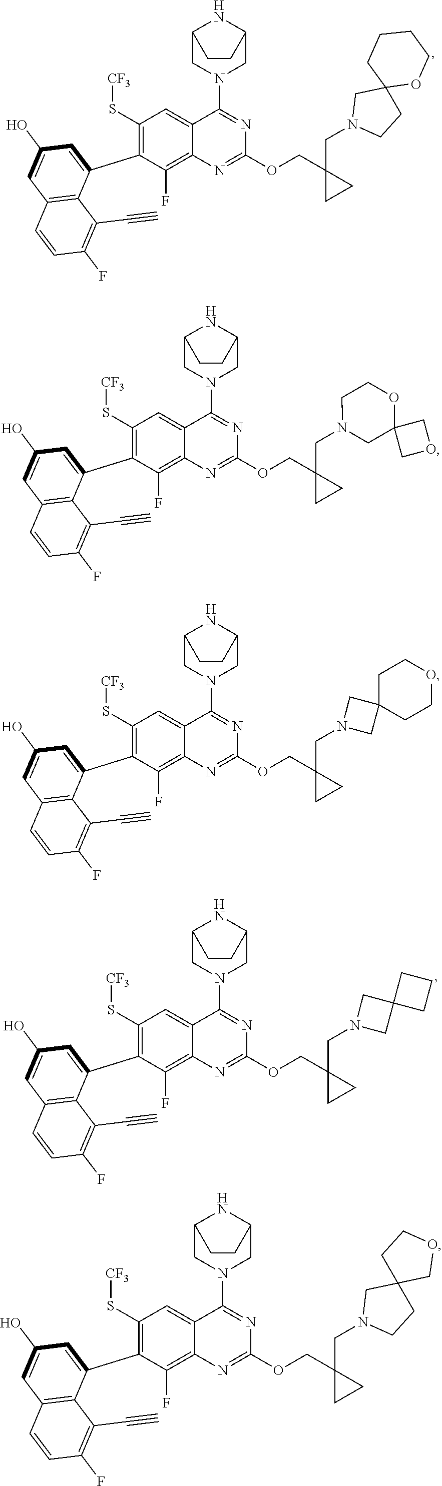

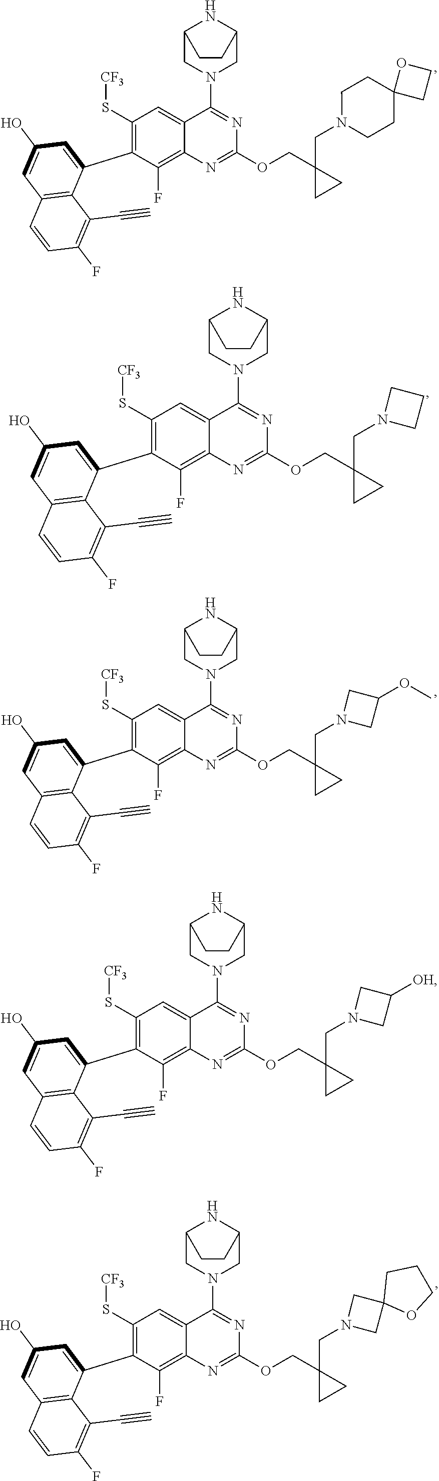

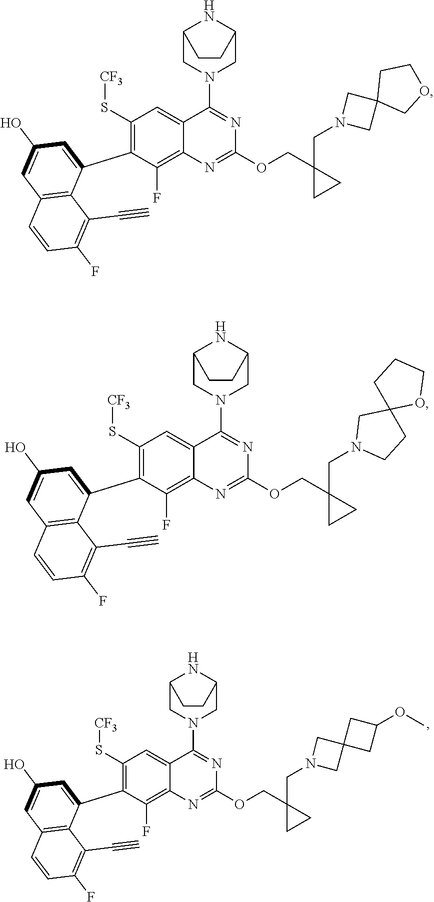

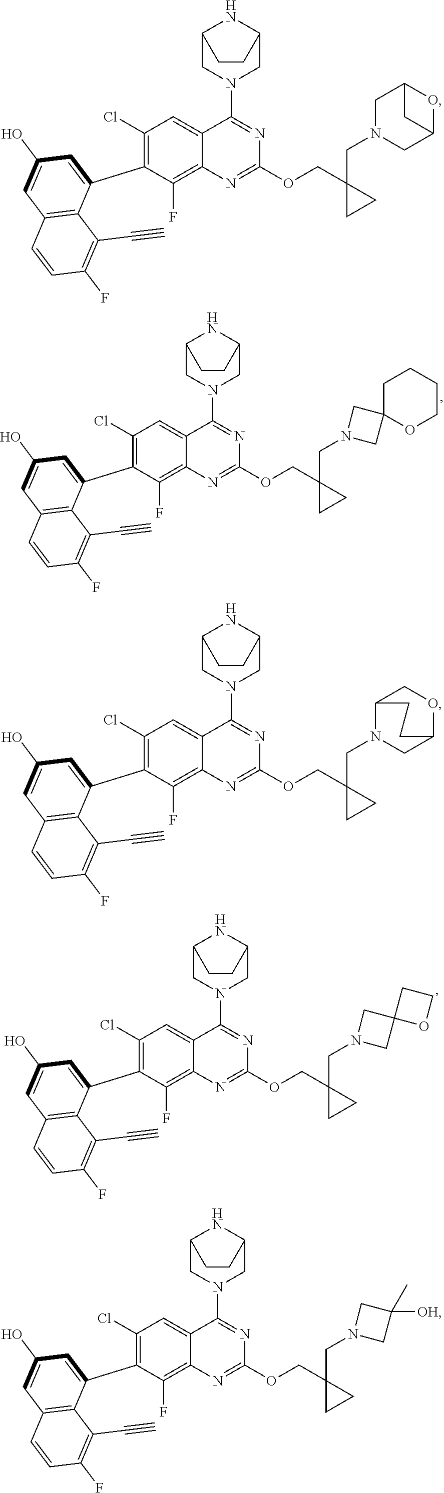

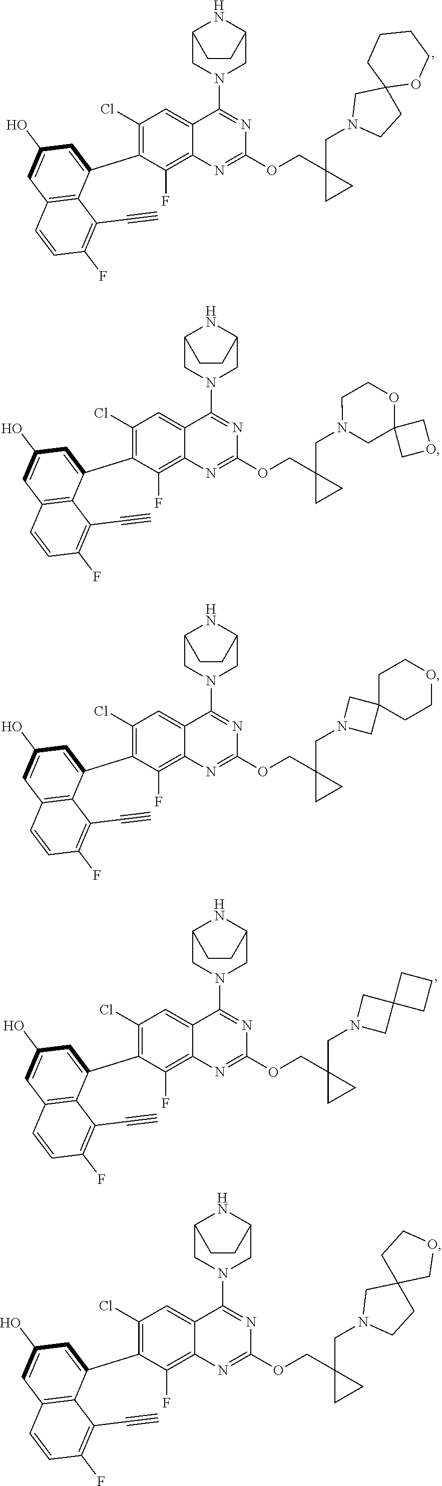

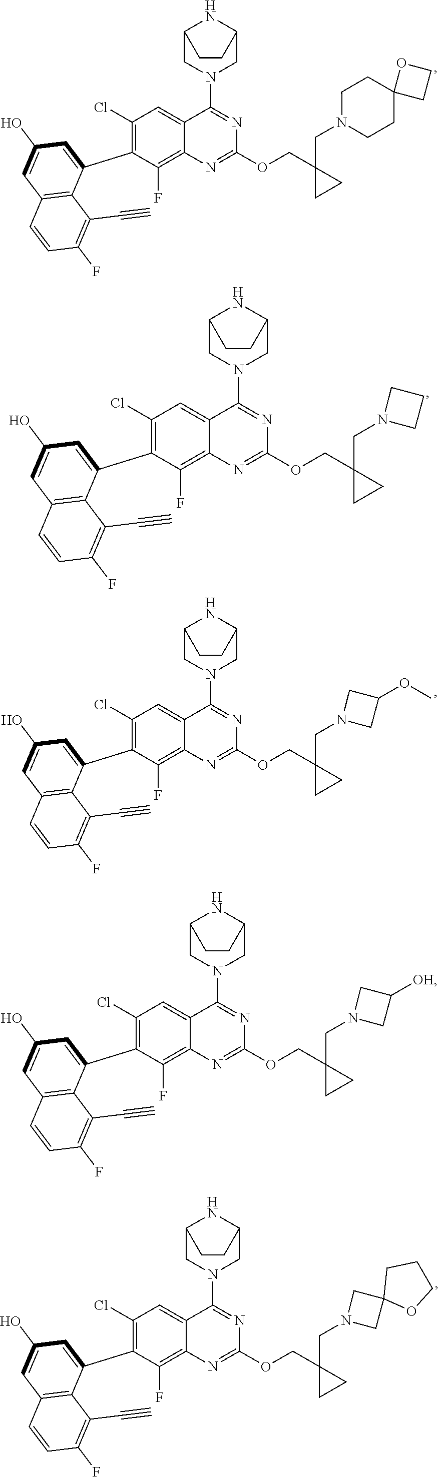

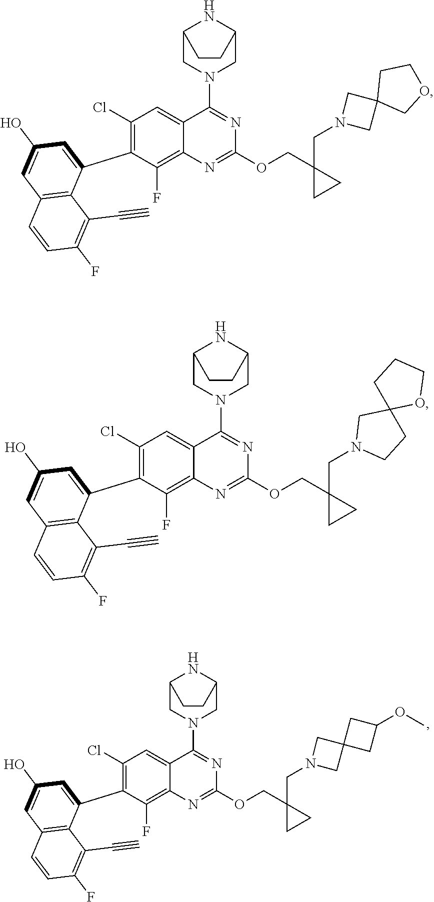

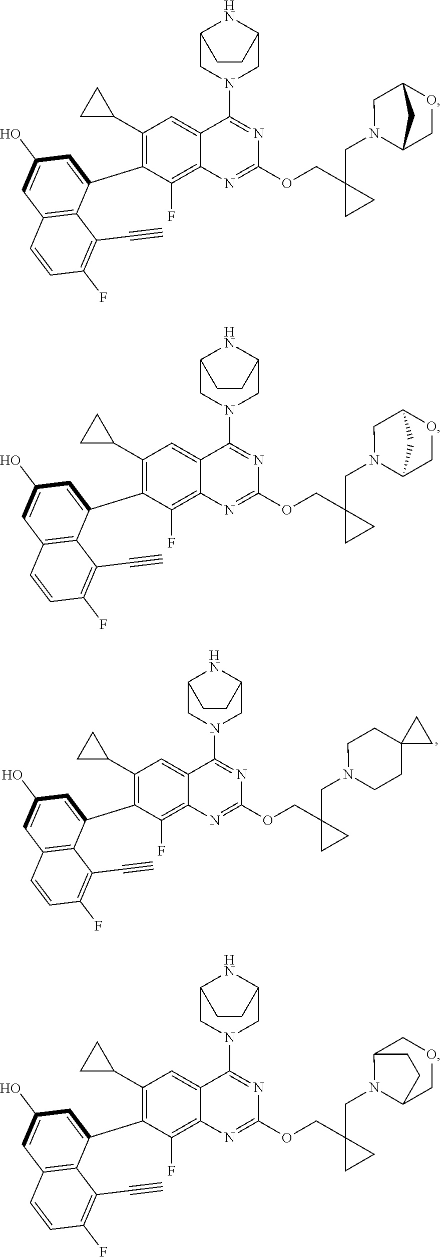

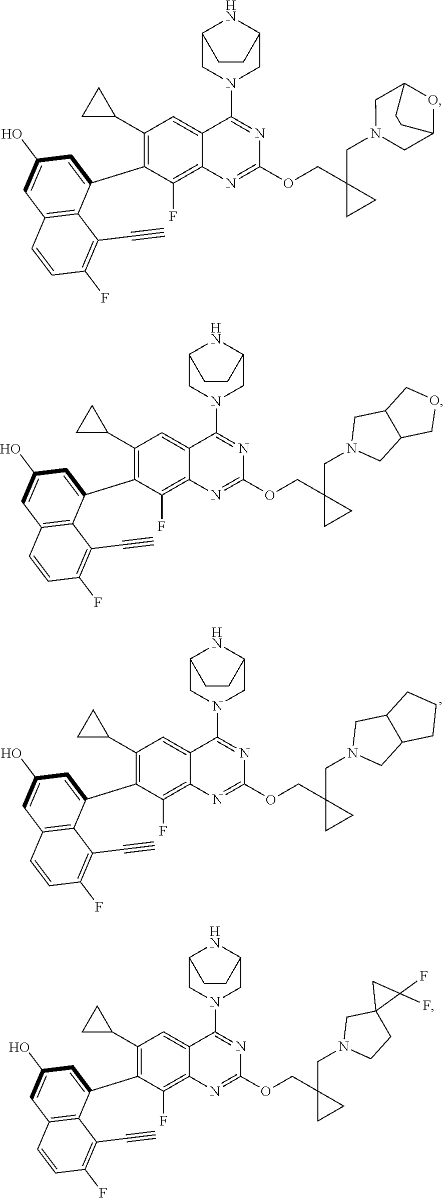

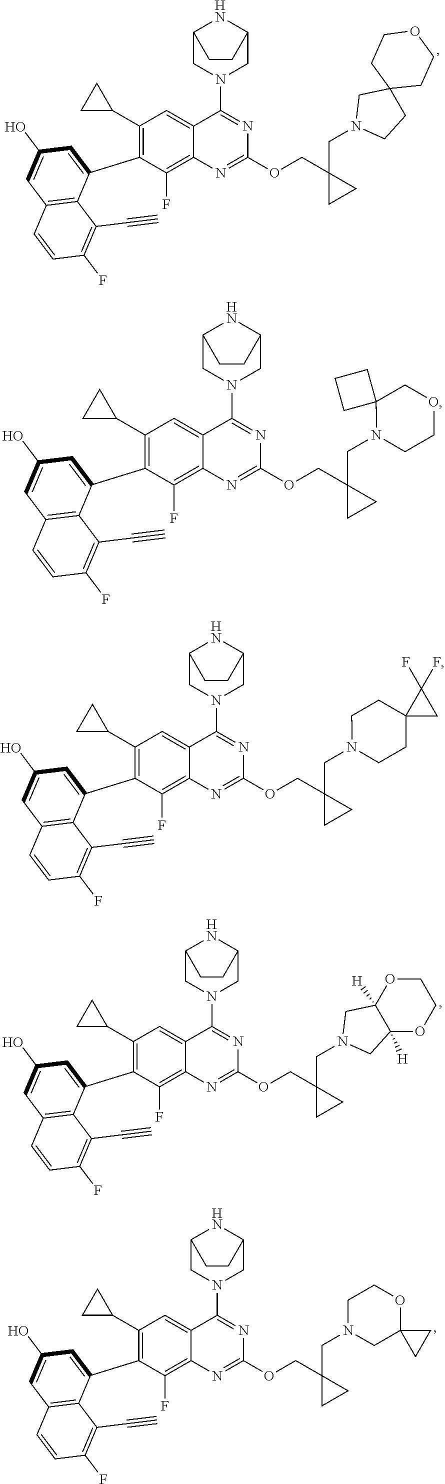

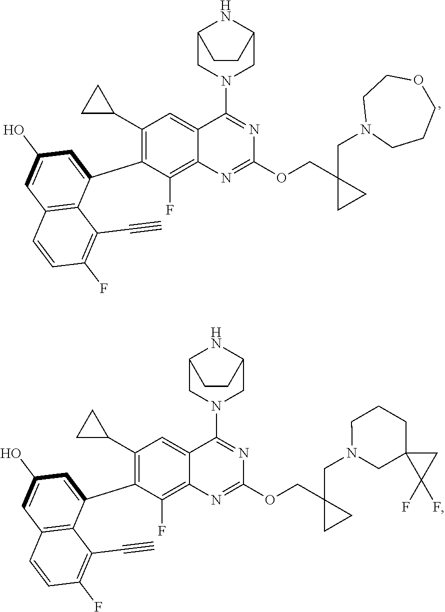

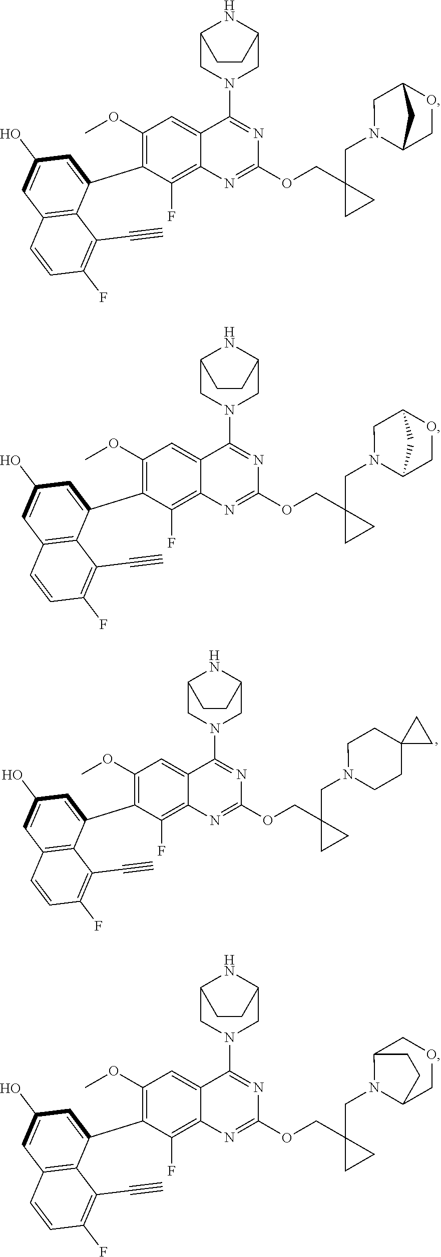

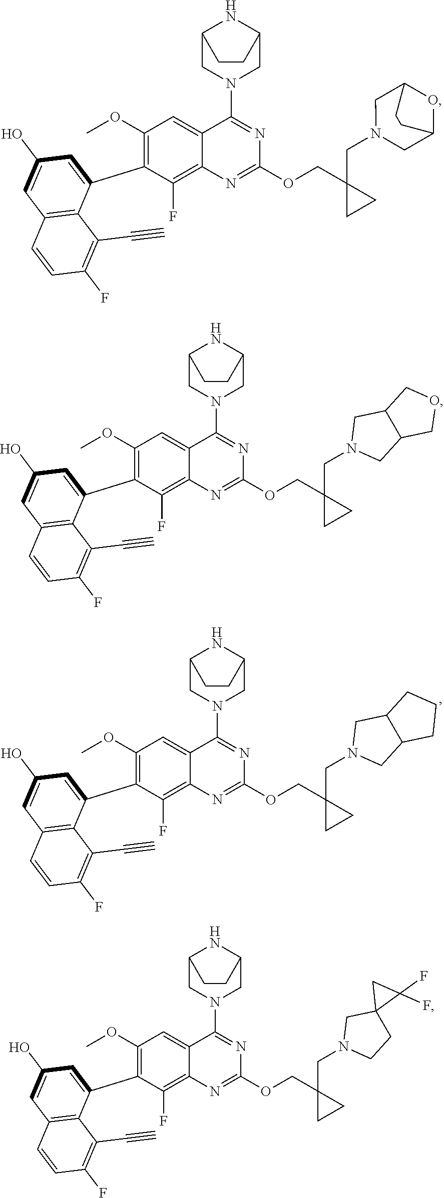

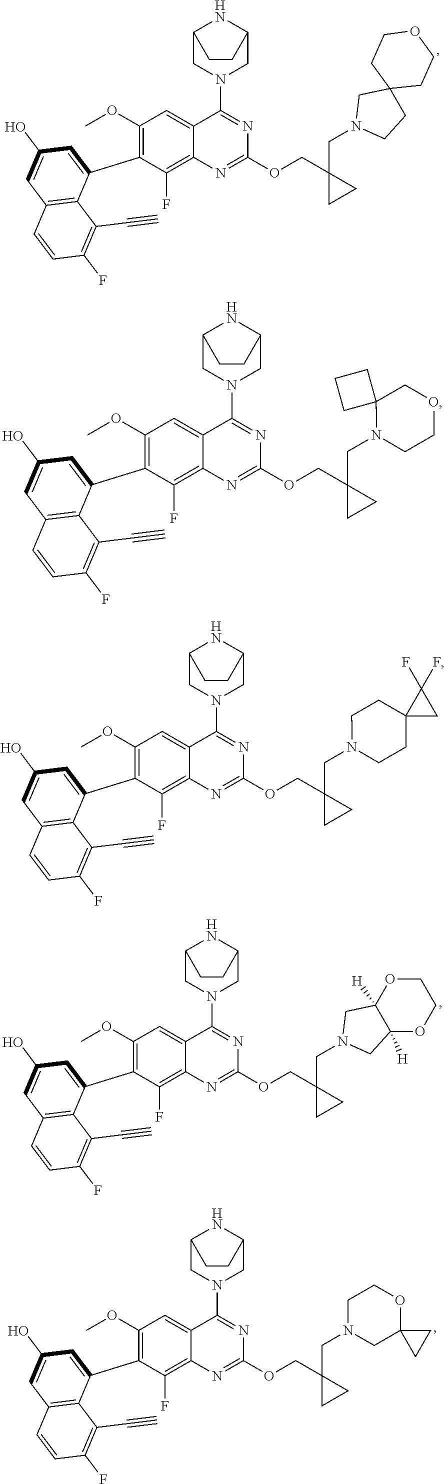

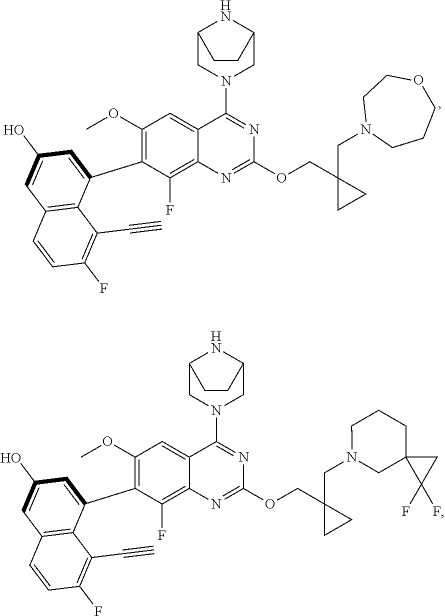

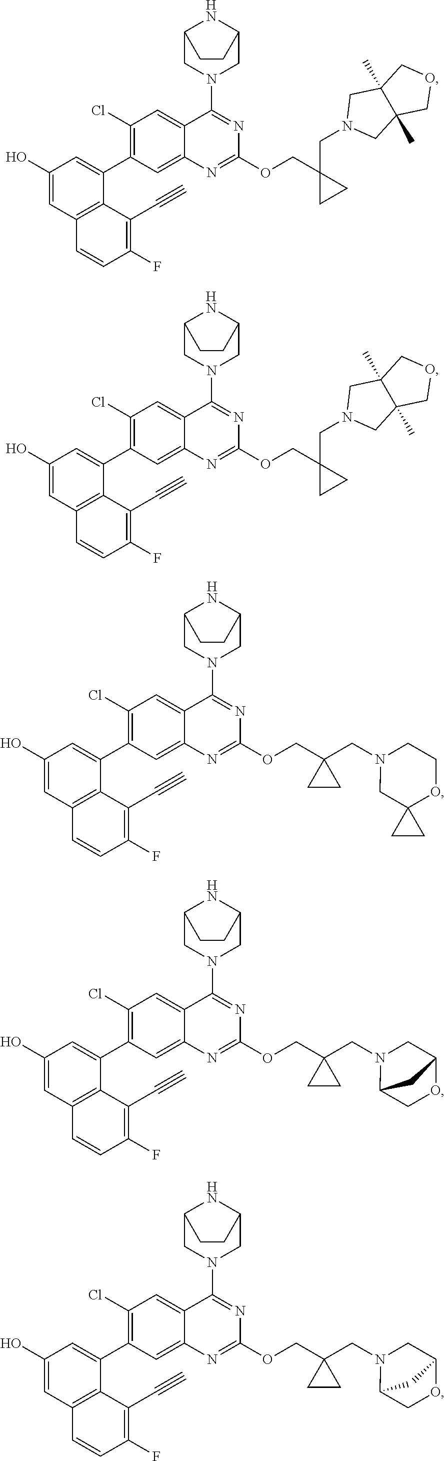 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3Cl)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3Cl)c12 ; C#Cc1c(F)ccc2cc(O)cc(-c3cc4nc(OCC5(CN6CCOC7(CC7)C6)CC5)nc(N5CC6CCC(C5)N6)c4cc3Cl)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3Cl)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3Cl)c12]

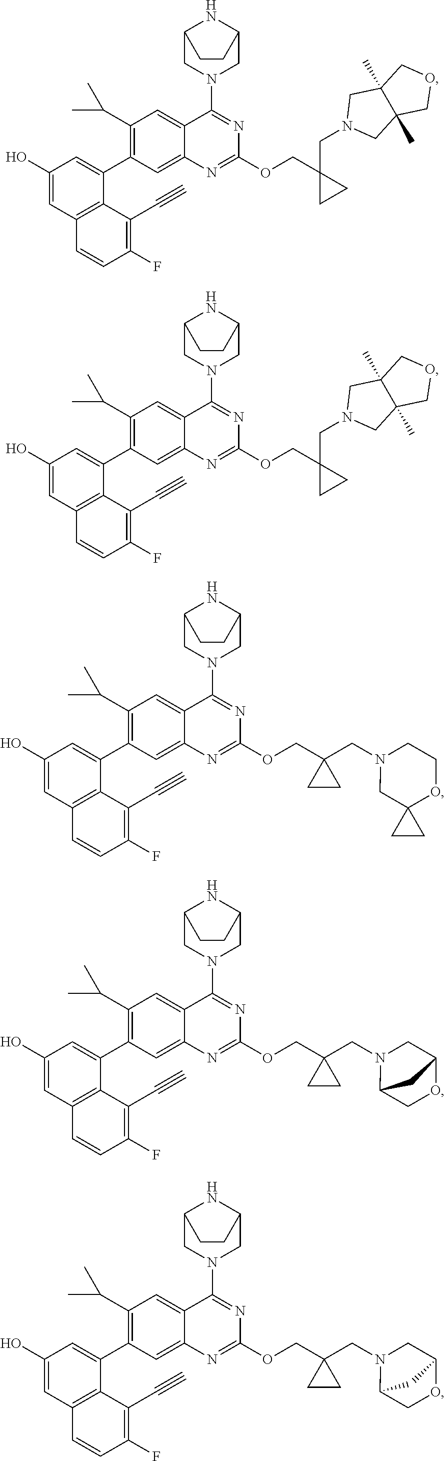 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3C(C)C)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3C(C)C)c12 ; C#Cc1c(F)ccc2cc(O)cc(-c3cc4nc(OCC5(CN6CCOC7(CC7)C6)CC5)nc(N5CC6CCC(C5)N6)c4cc3C(C)C)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3C(C)C)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3C(C)C)c12]

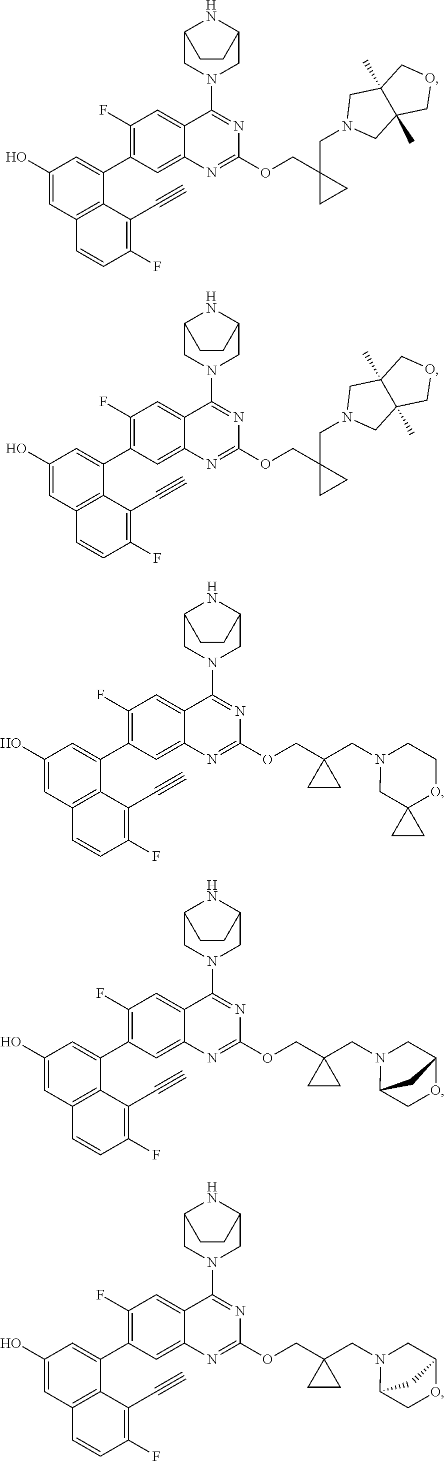 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3F)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3F)c12 ; C#Cc1c(F)ccc2cc(O)cc(-c3cc4nc(OCC5(CN6CCOC7(CC7)C6)CC5)nc(N5CC6CCC(C5)N6)c4cc3F)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3F)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3F)c12]

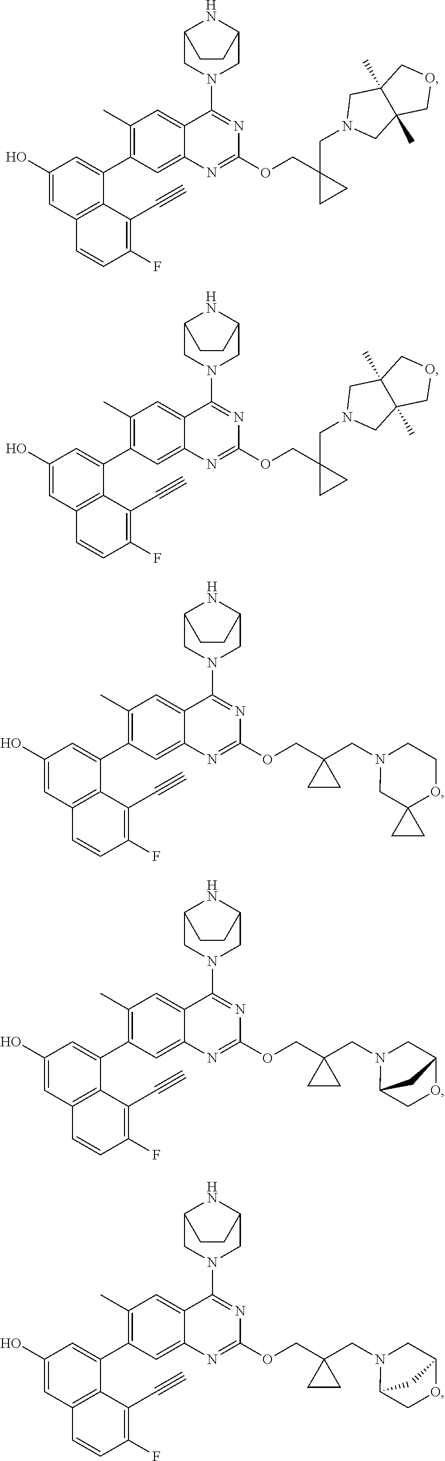 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3C)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3C)c12 ; C#Cc1c(F)ccc2cc(O)cc(-c3cc4nc(OCC5(CN6CCOC7(CC7)C6)CC5)nc(N5CC6CCC(C5)N6)c4cc3C)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3C)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3C)c12]

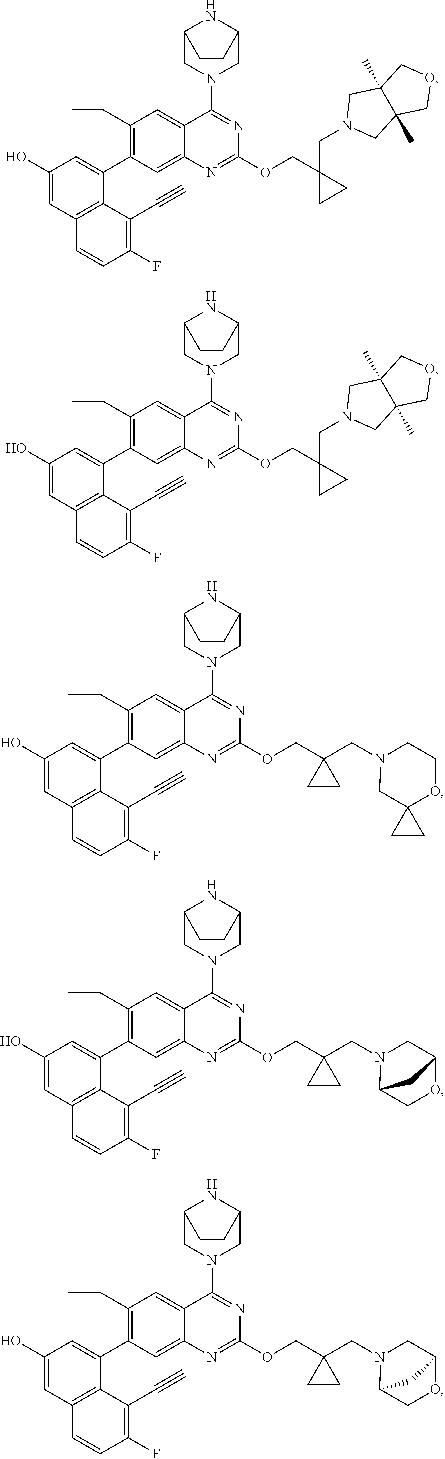 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3CC)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3CC)c12 ; C#Cc1c(F)ccc2cc(O)cc(-c3cc4nc(OCC5(CN6CCOC7(CC7)C6)CC5)nc(N5CC6CCC(C5)N6)c4cc3CC)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3CC)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3CC)c12]

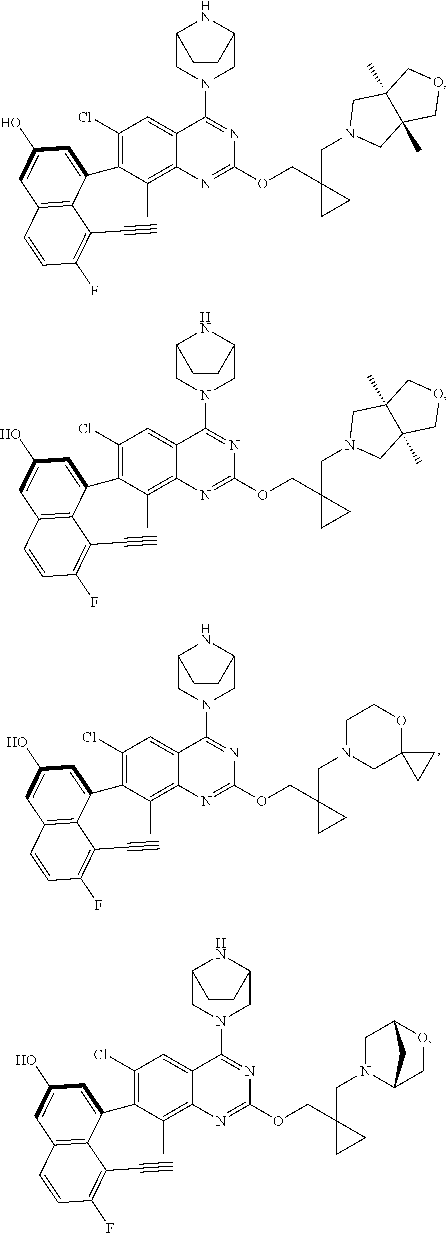

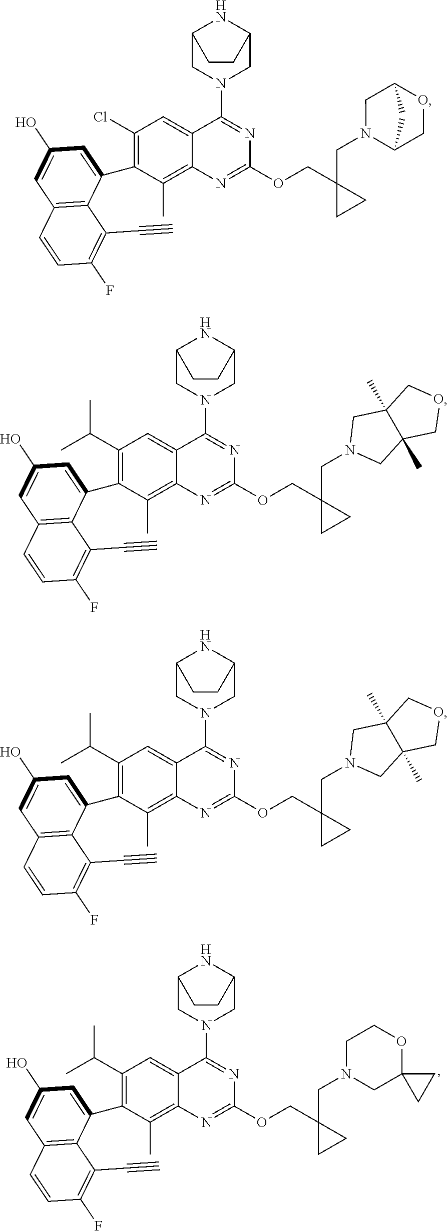

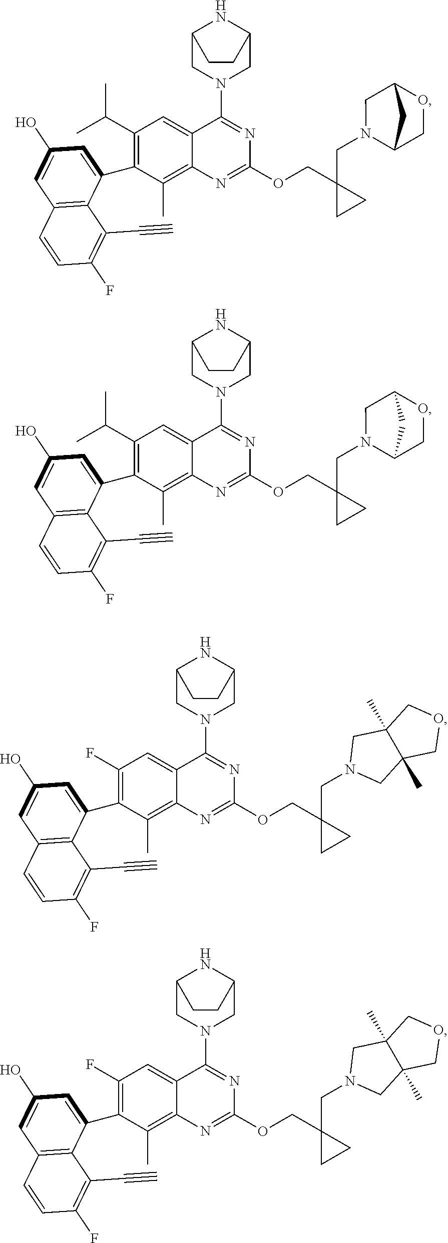

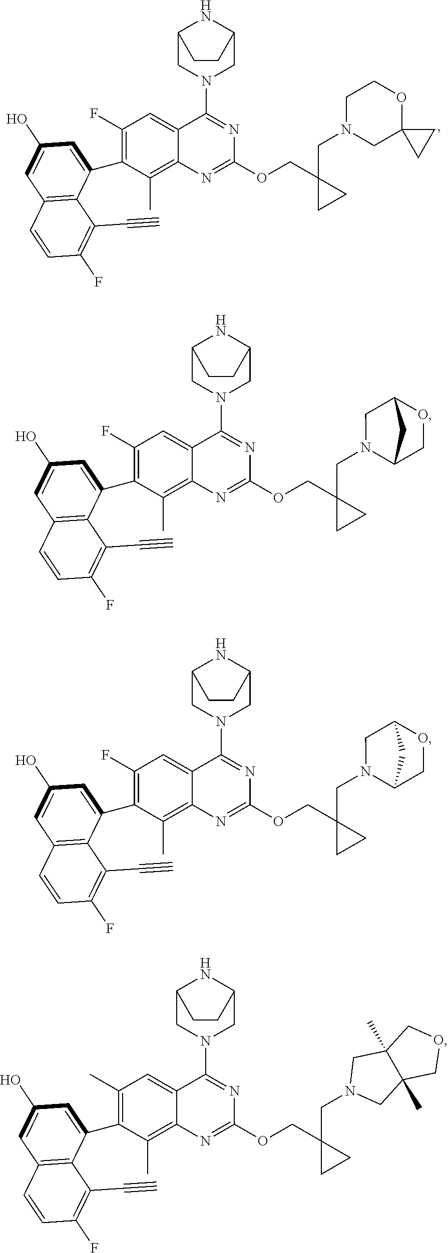

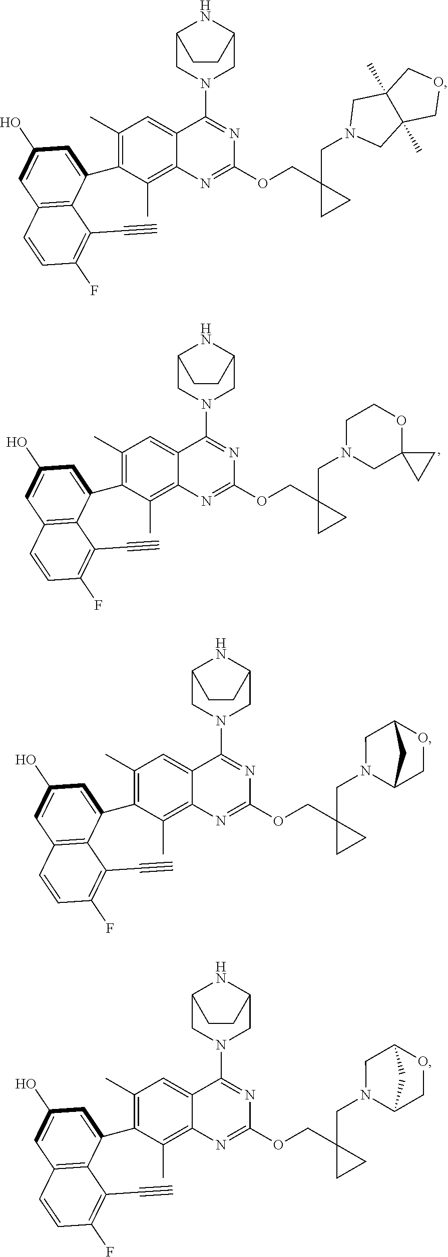

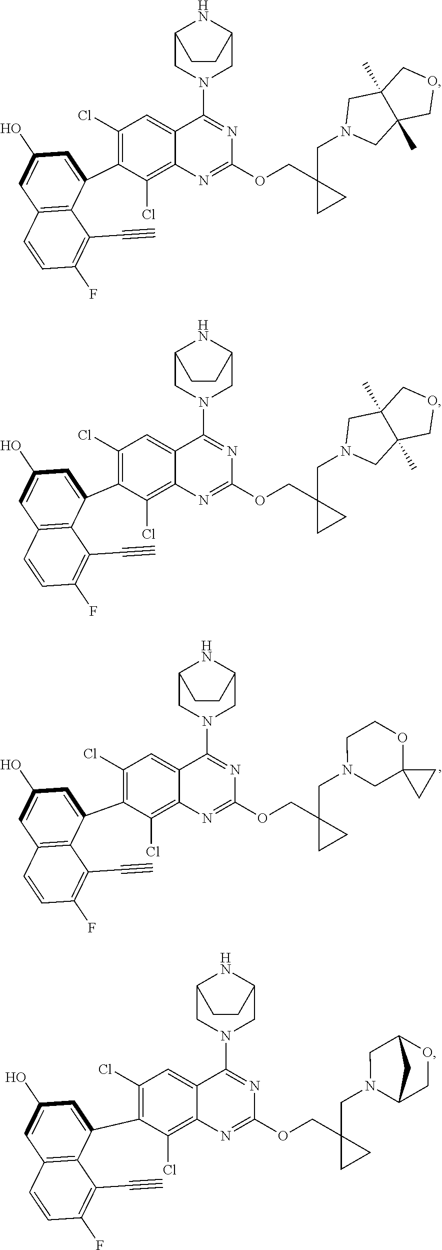

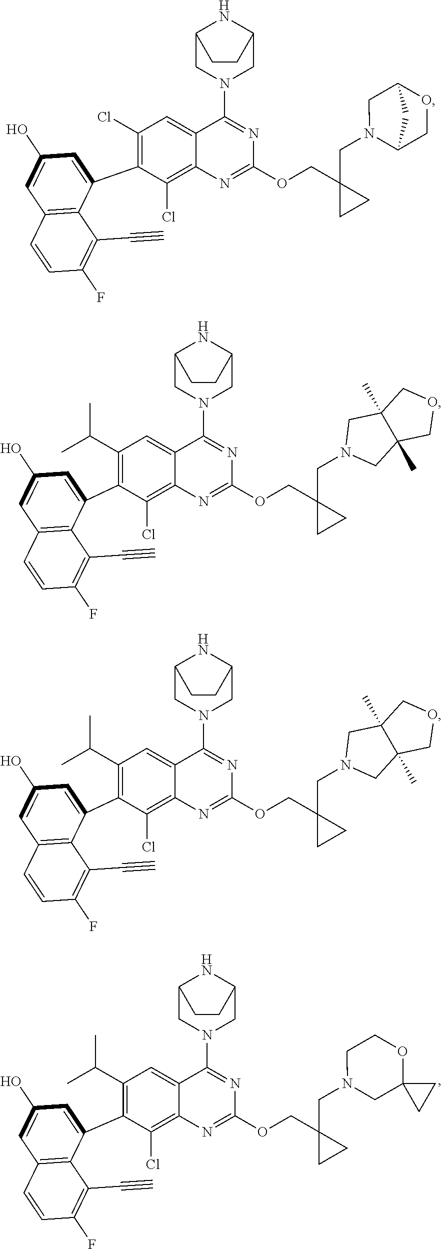

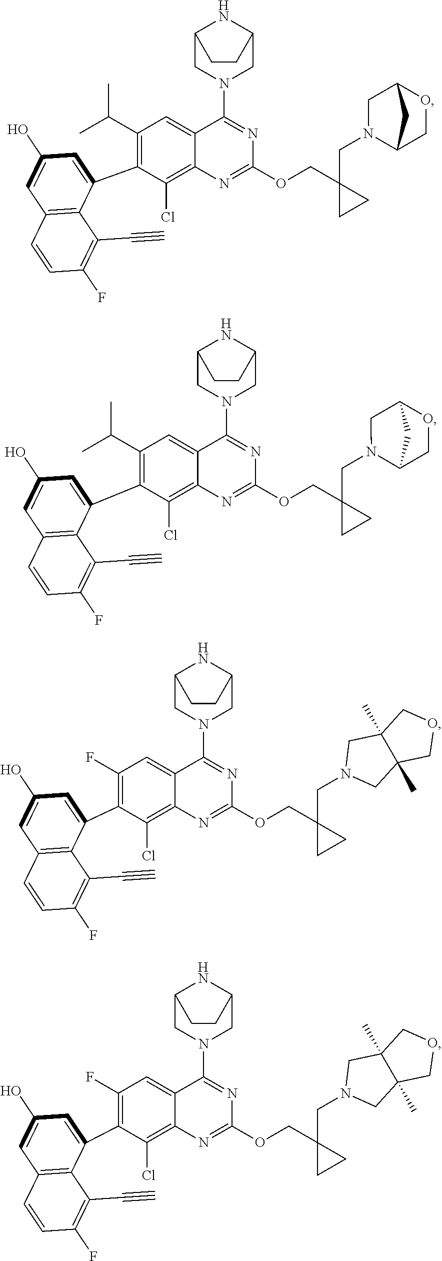

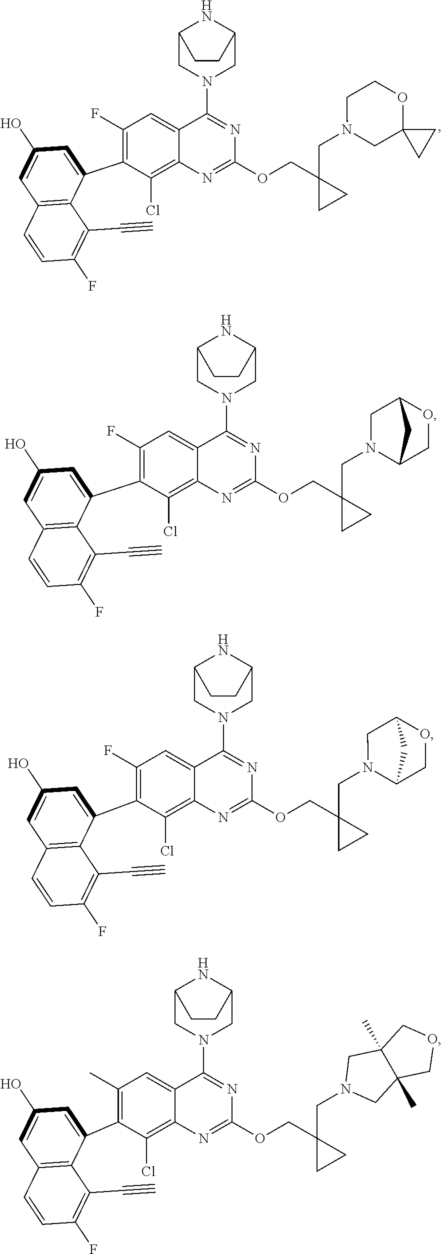

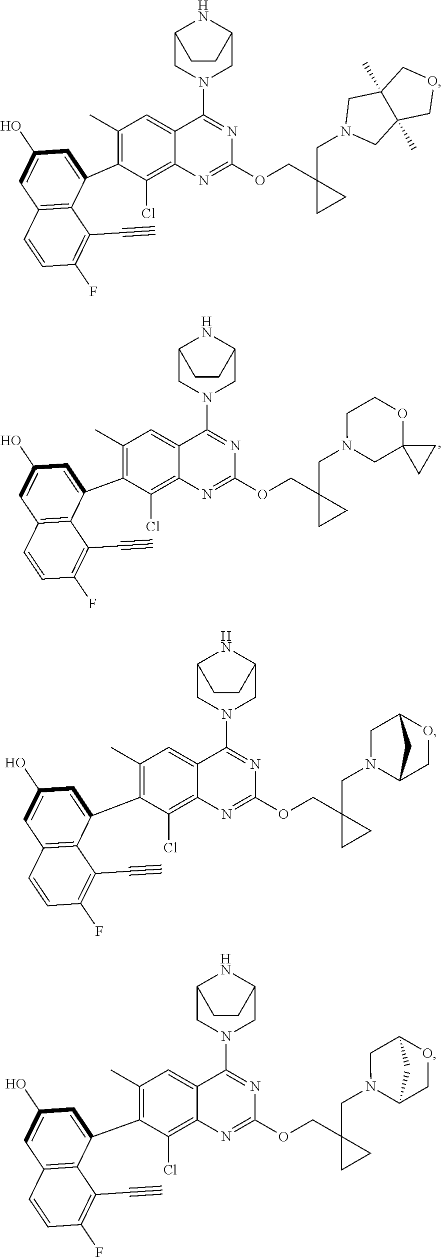

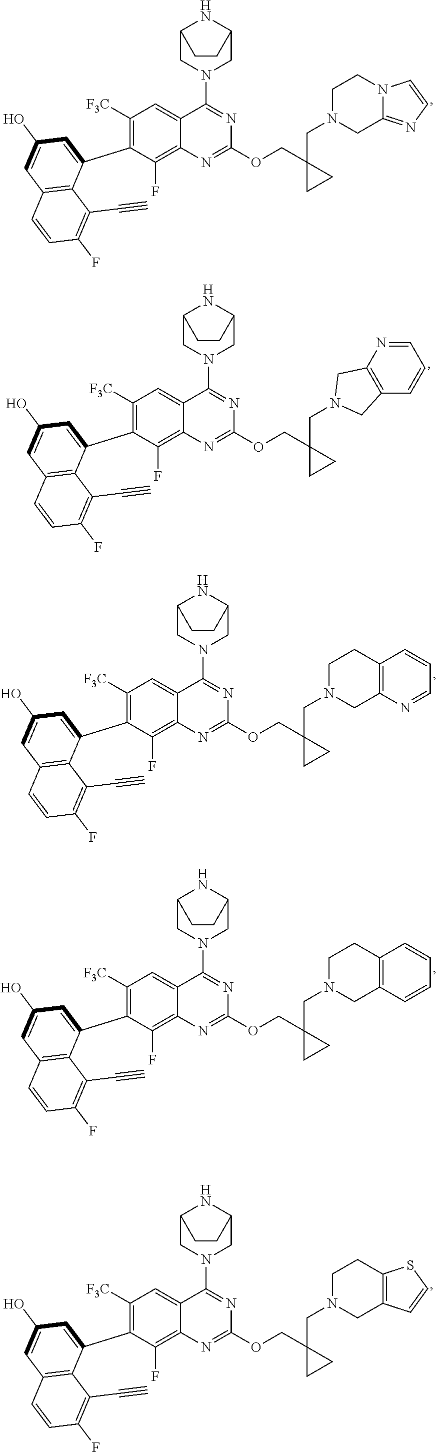

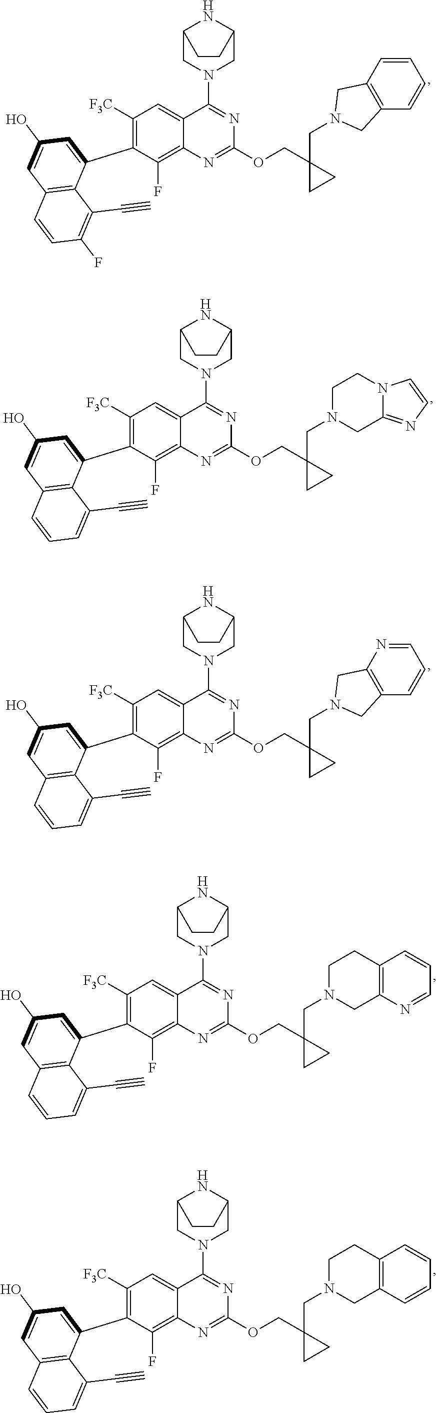

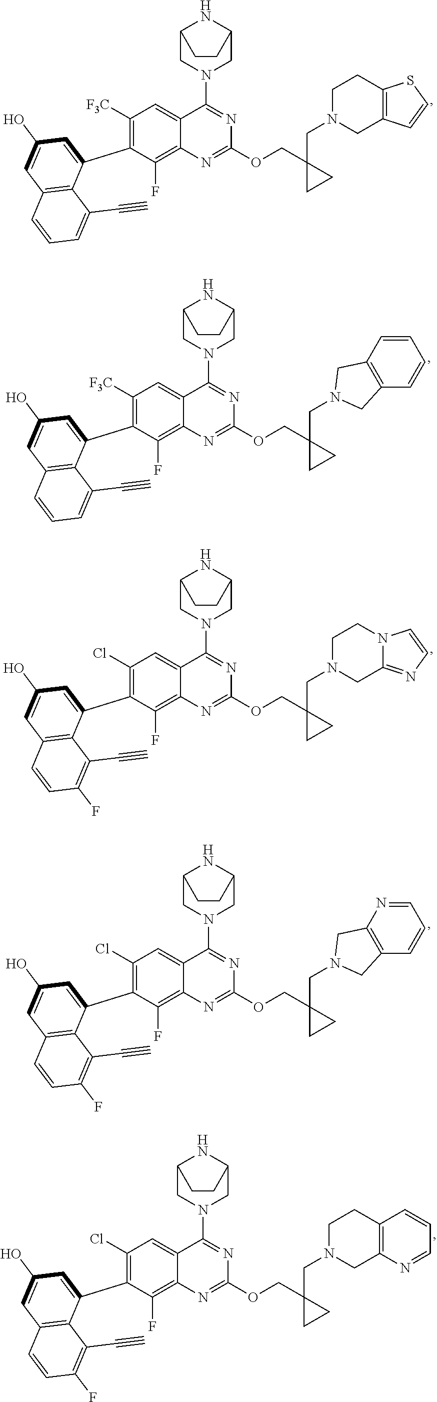

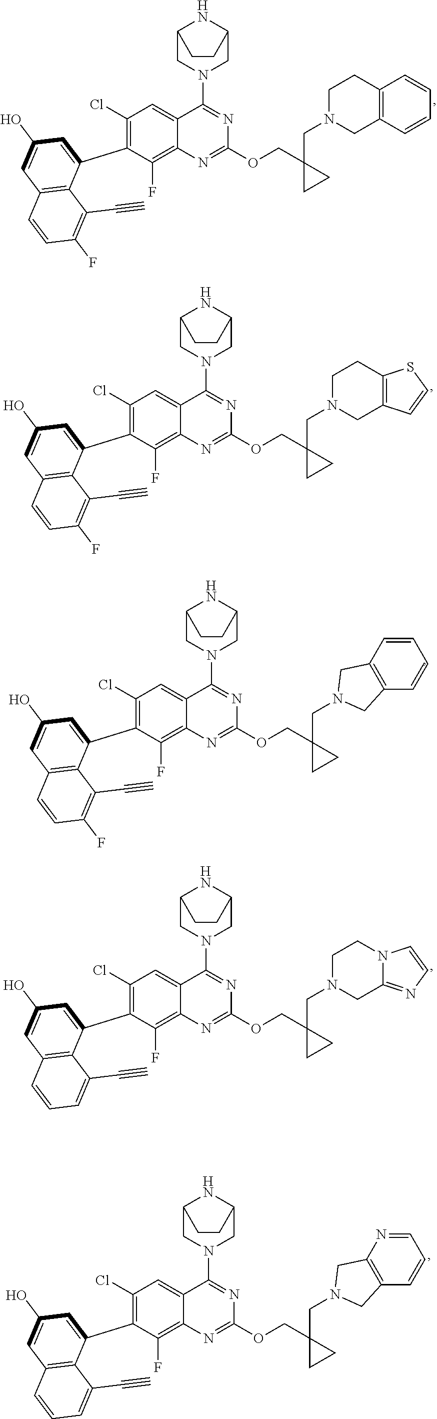

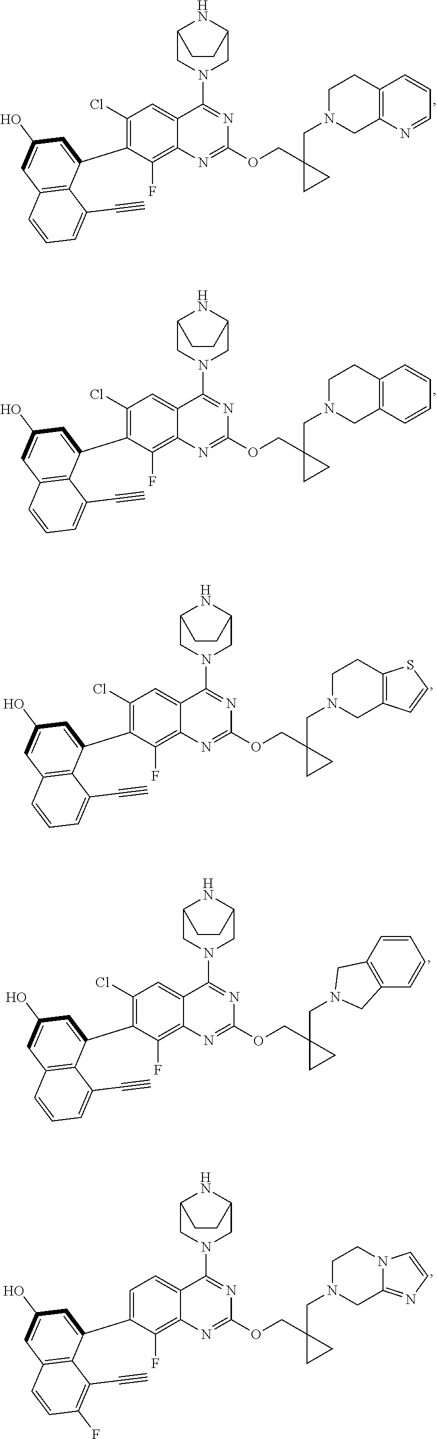

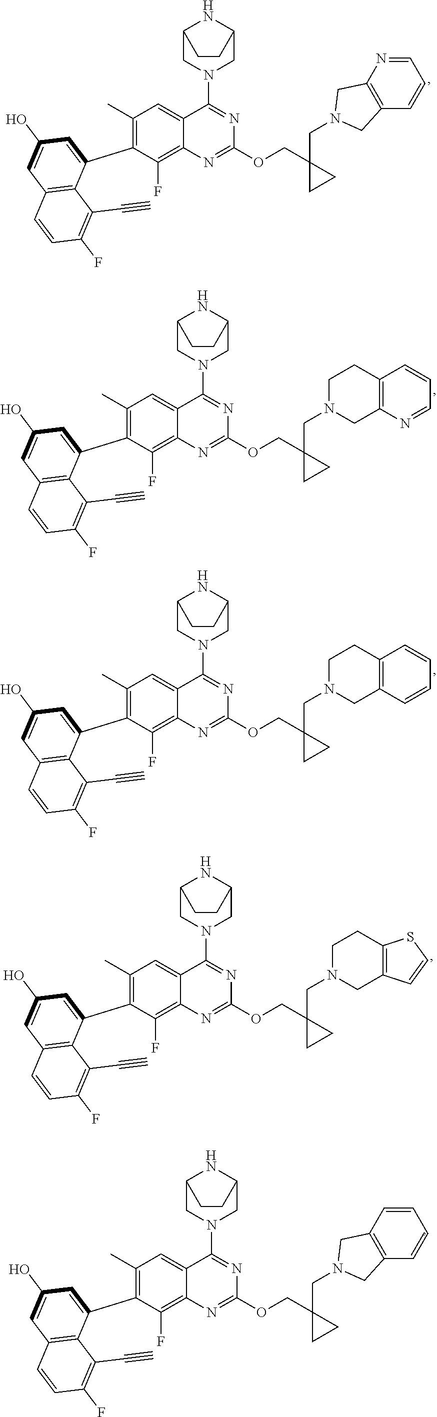

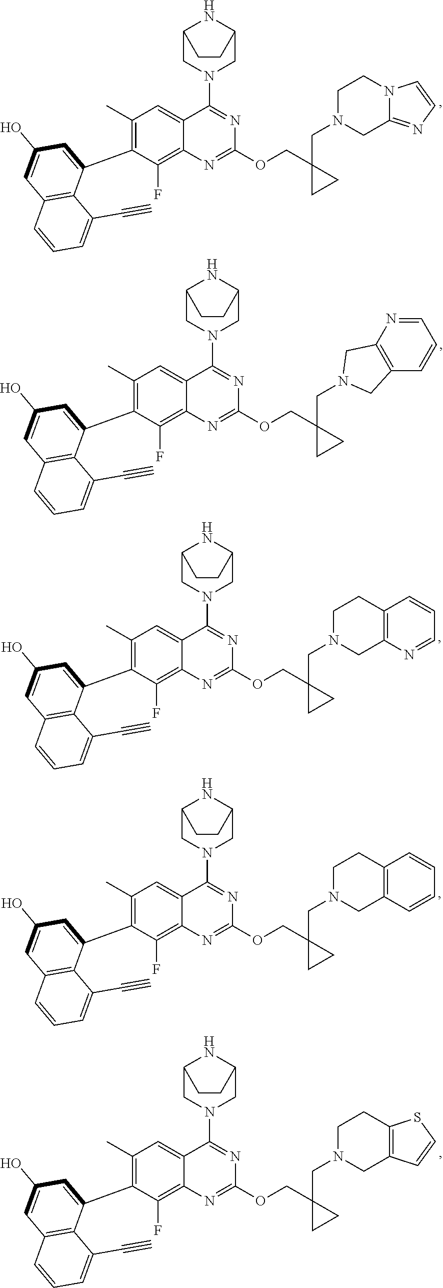

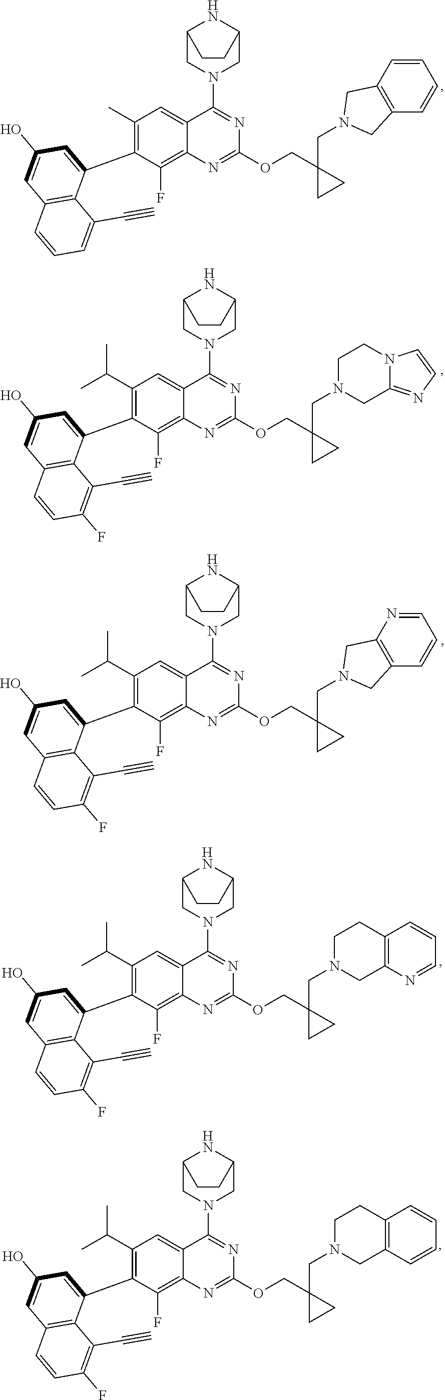

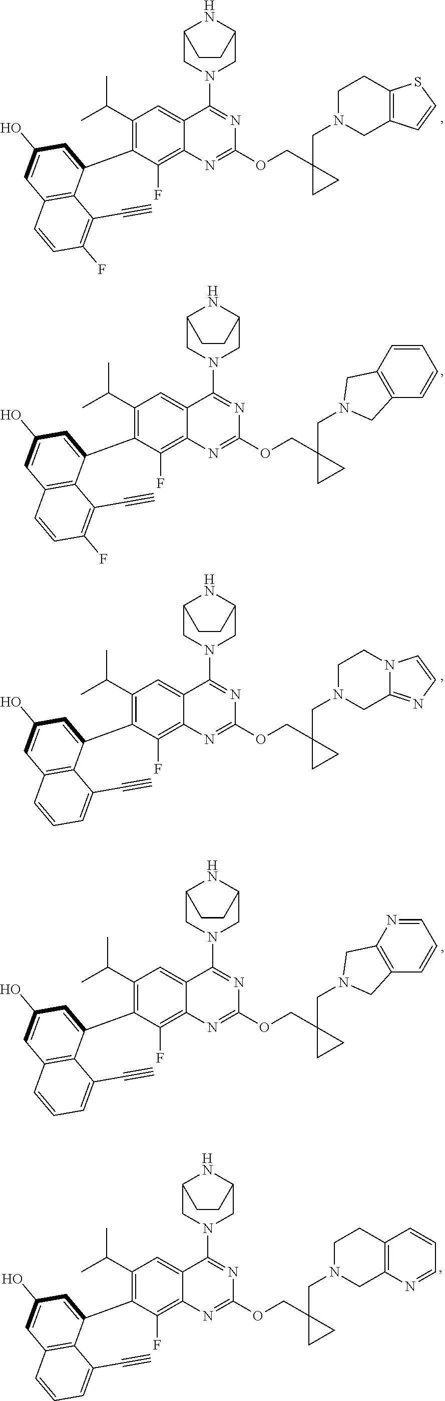

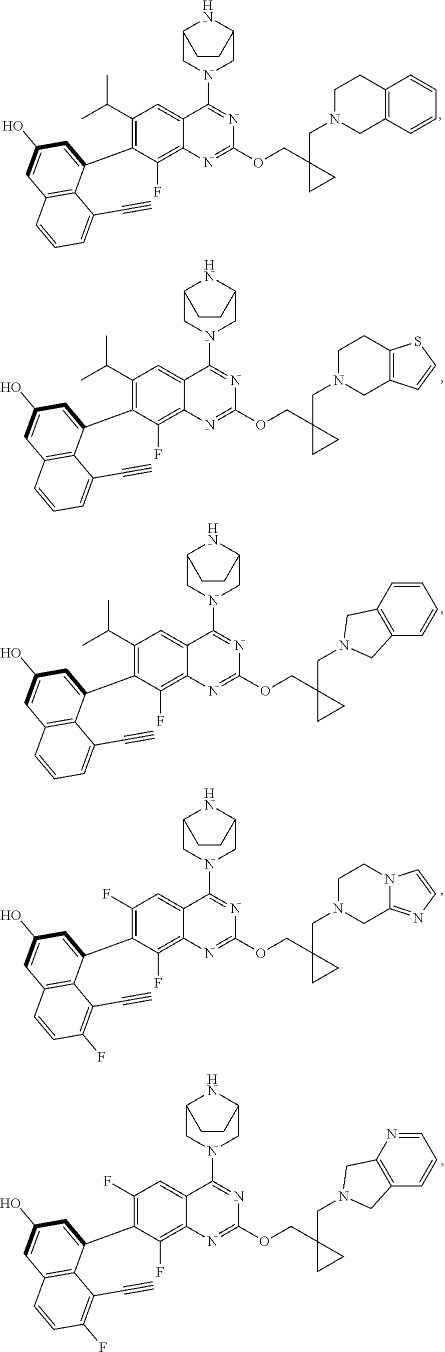

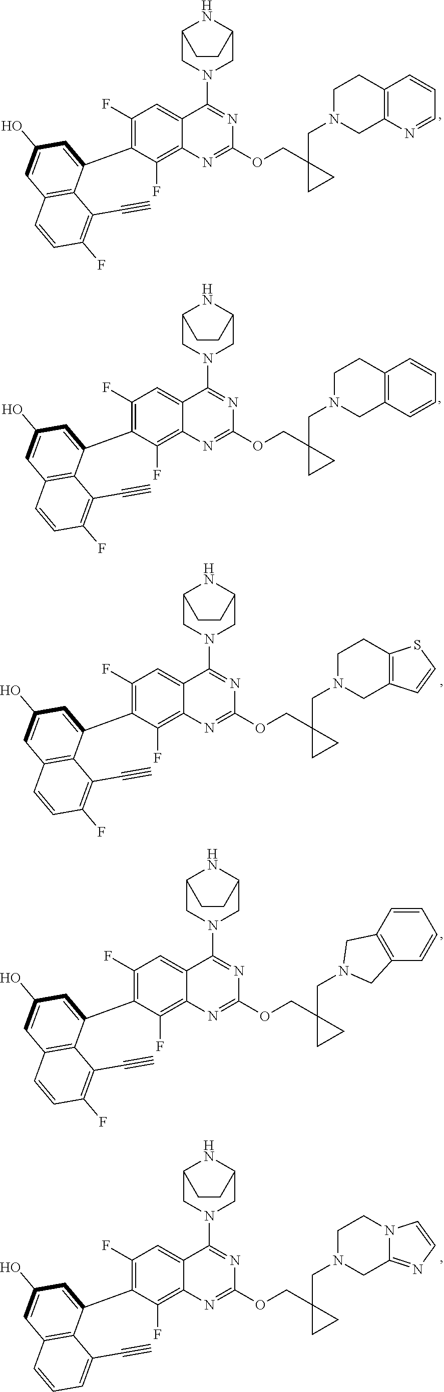

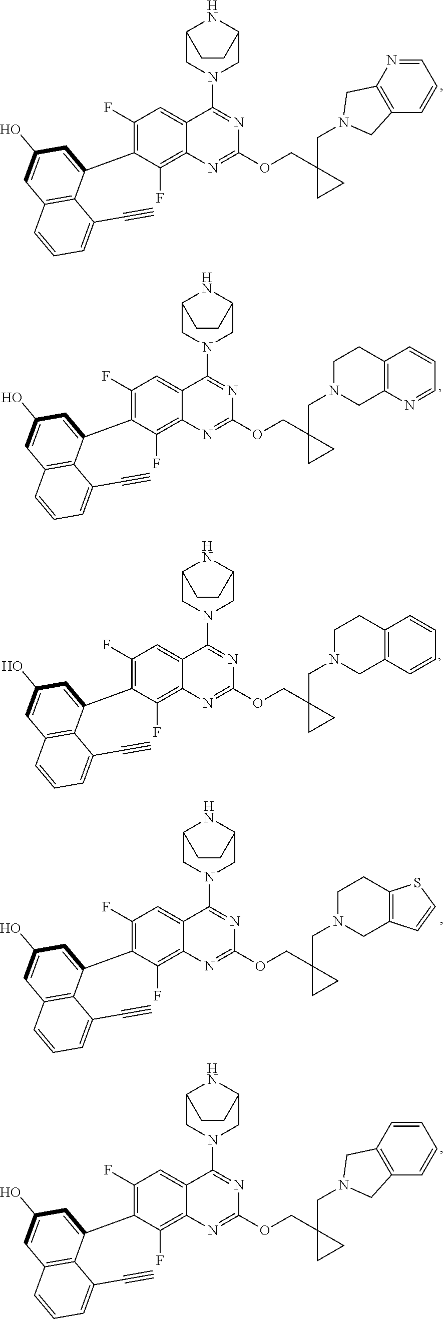

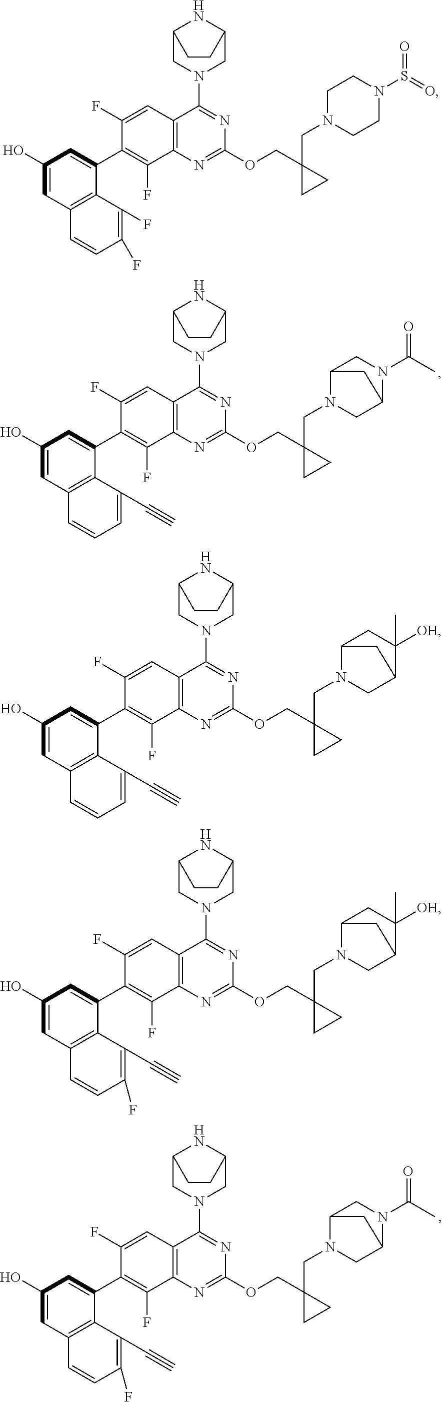

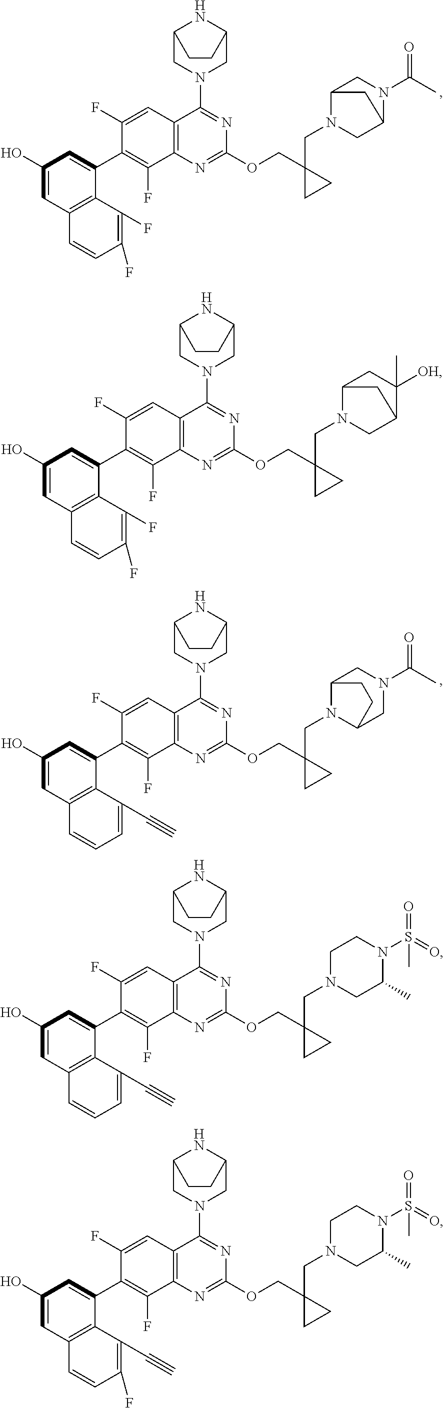

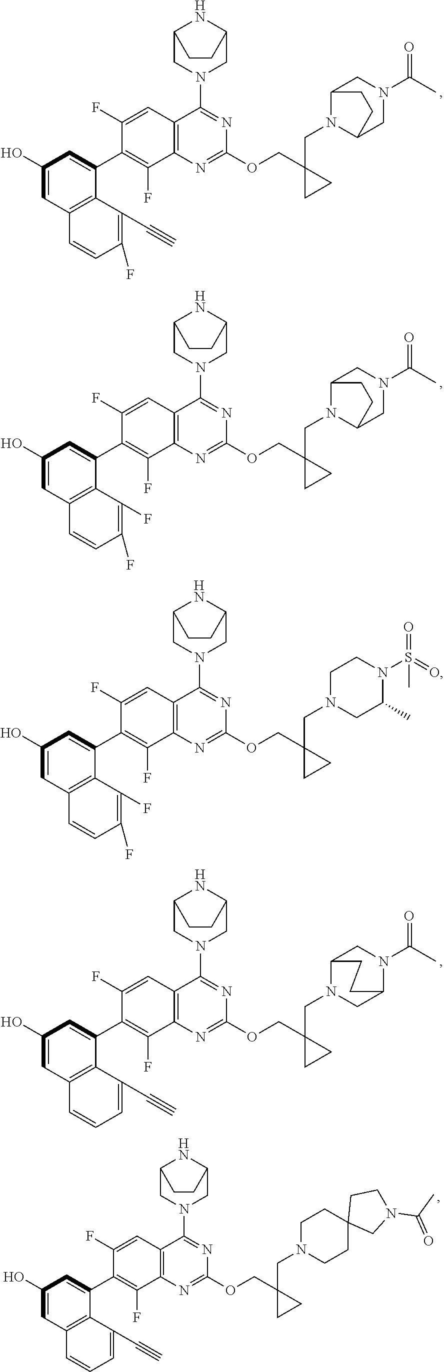

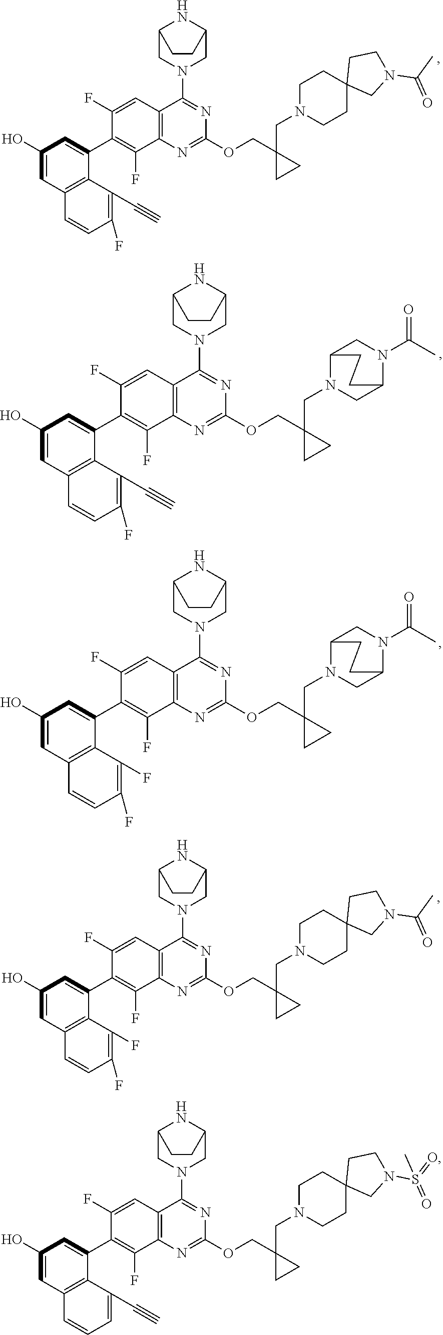

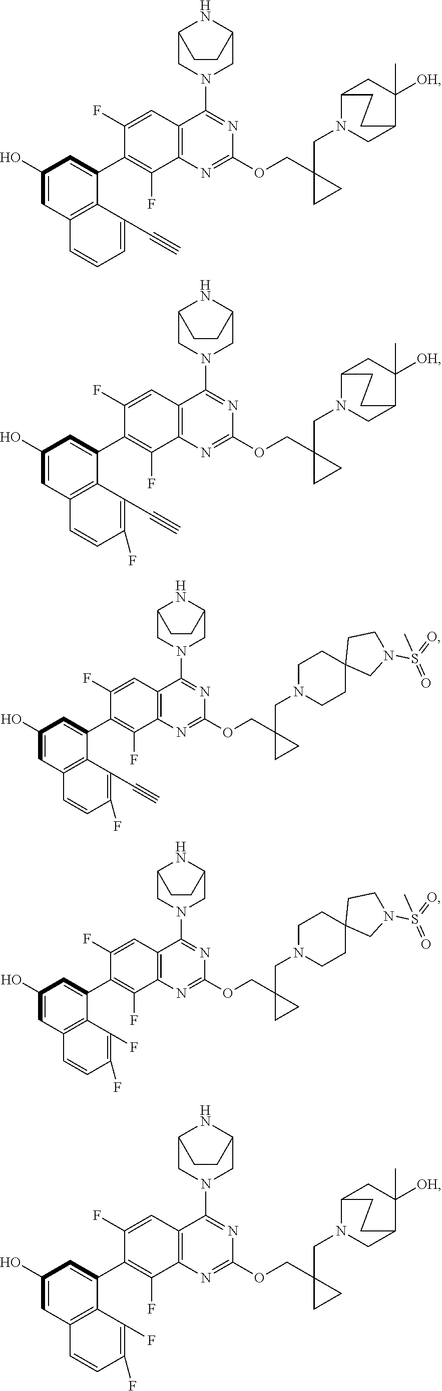

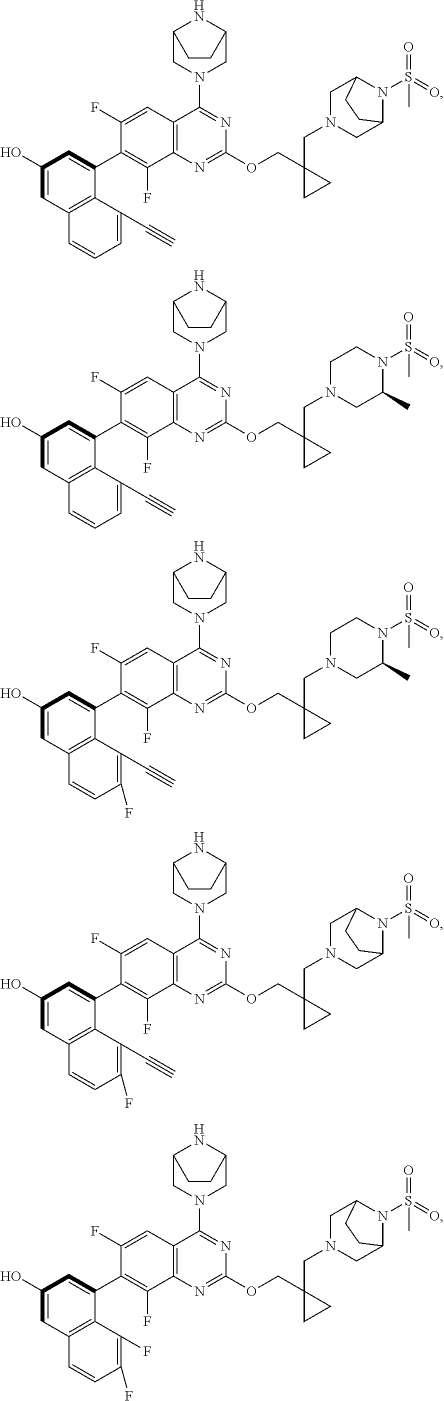

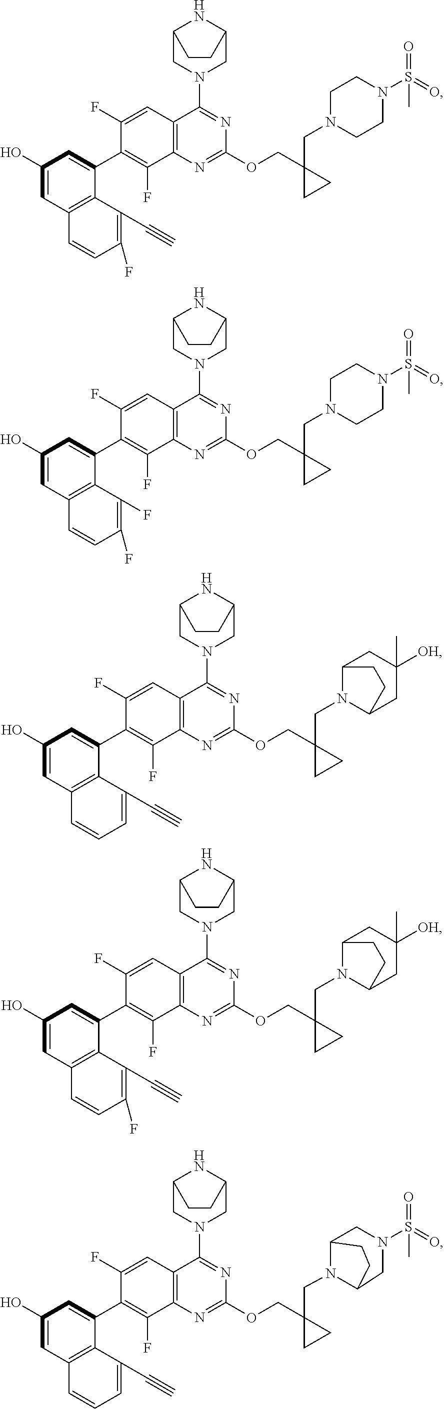

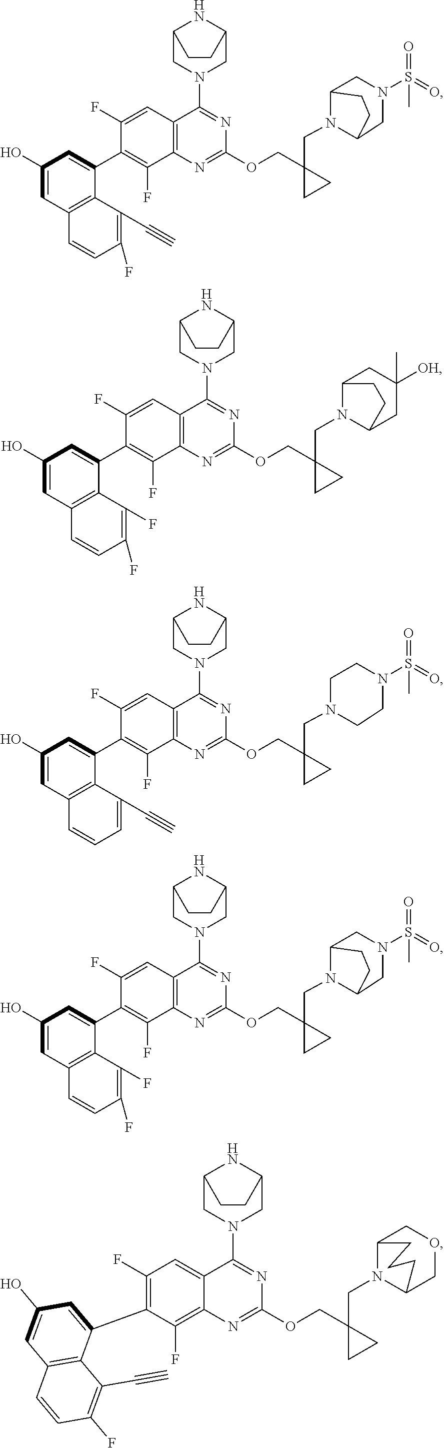

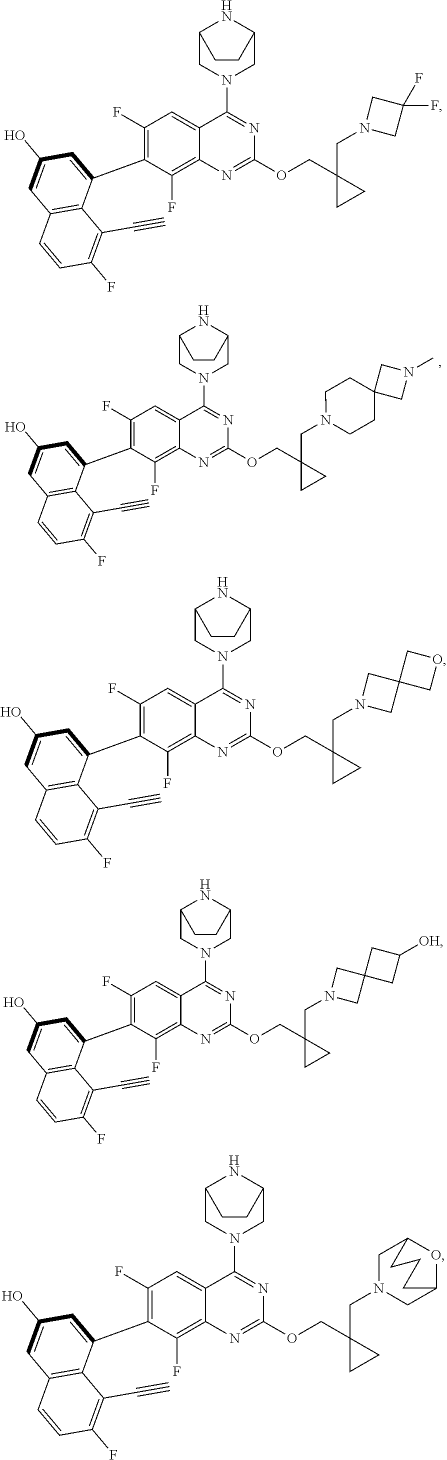

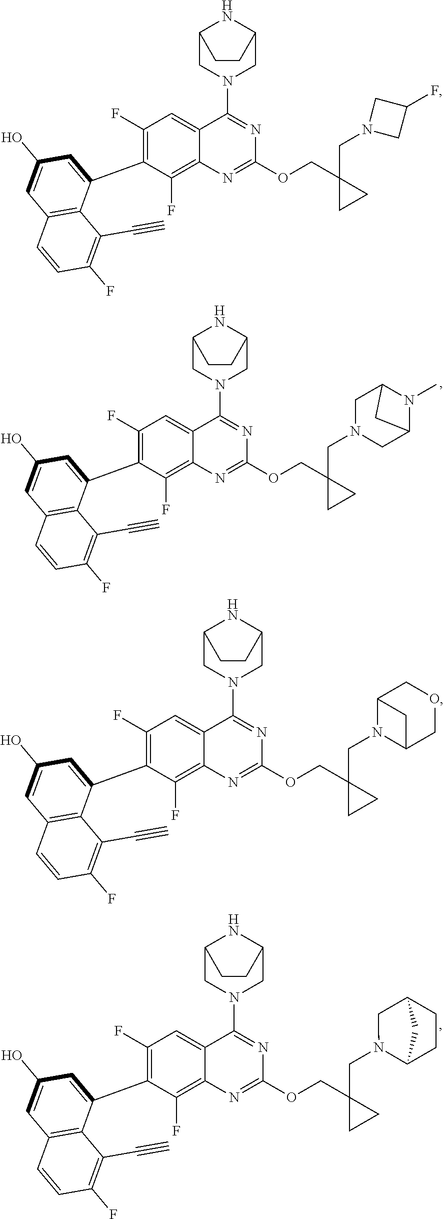

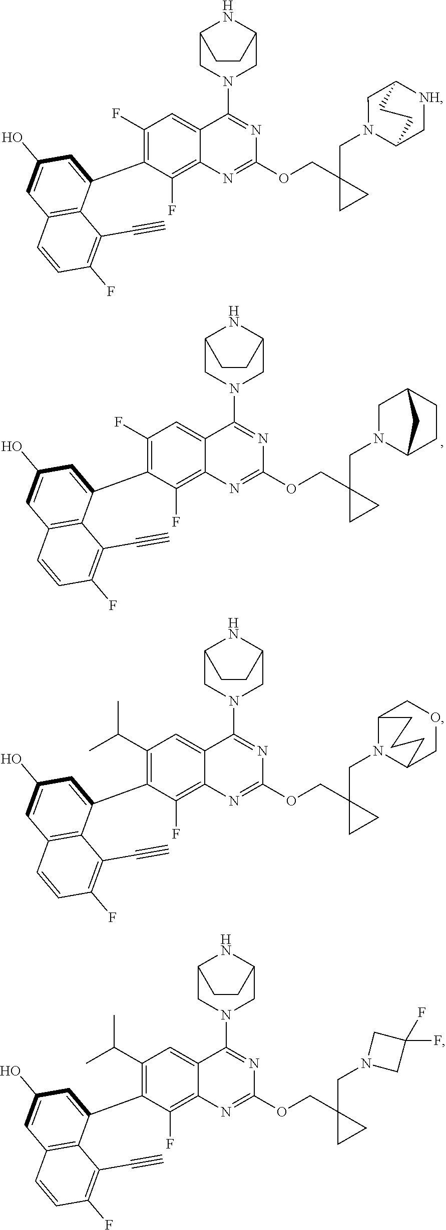

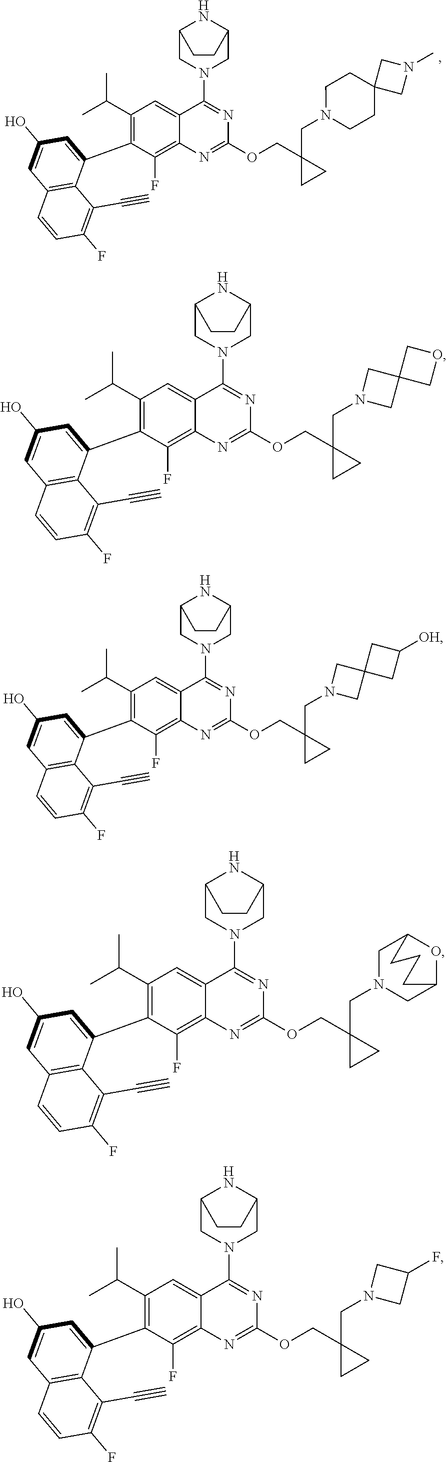

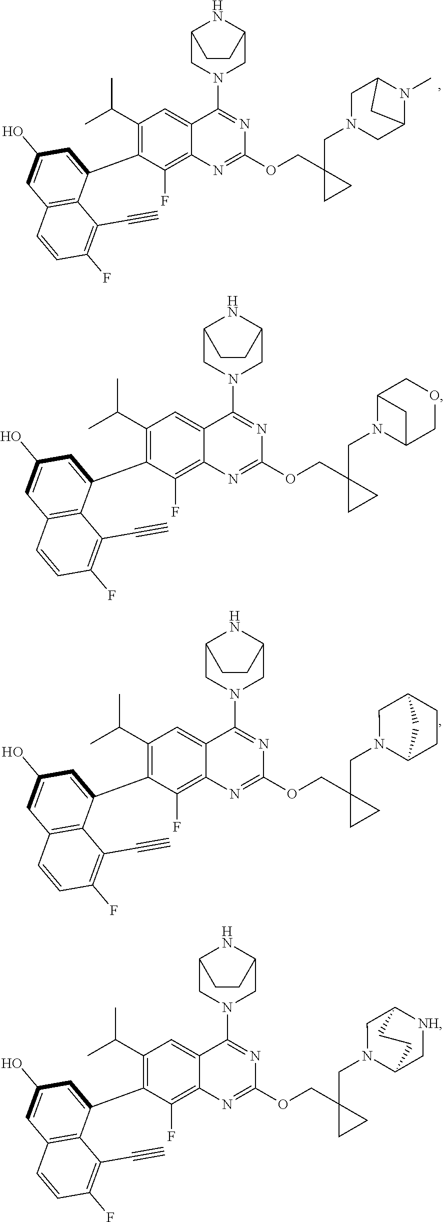

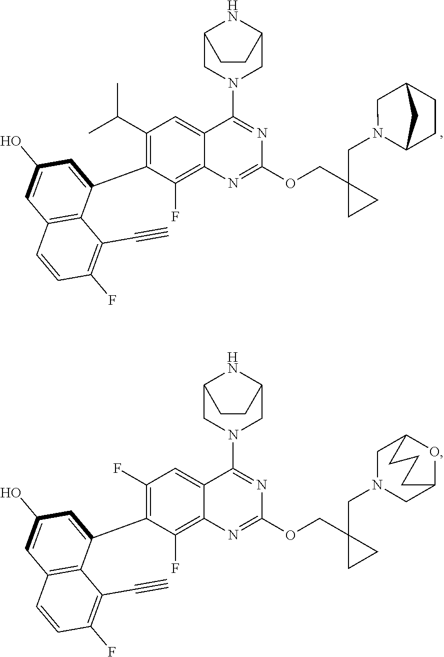

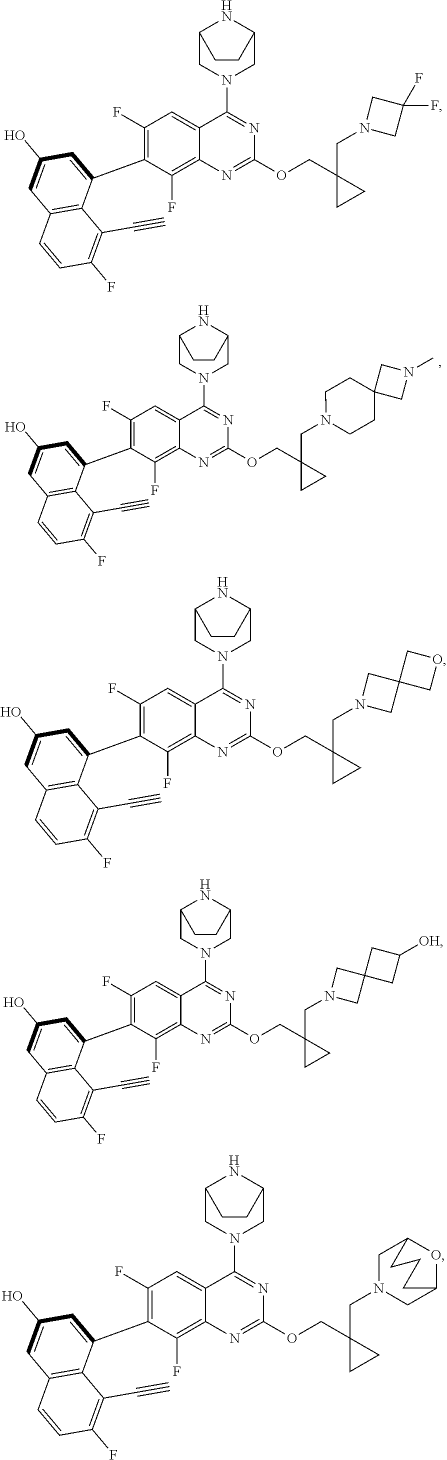

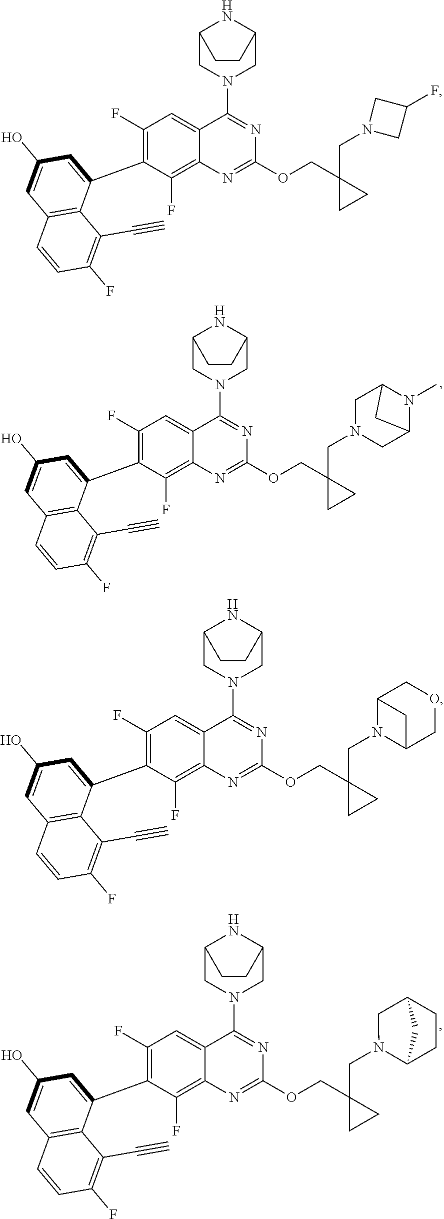

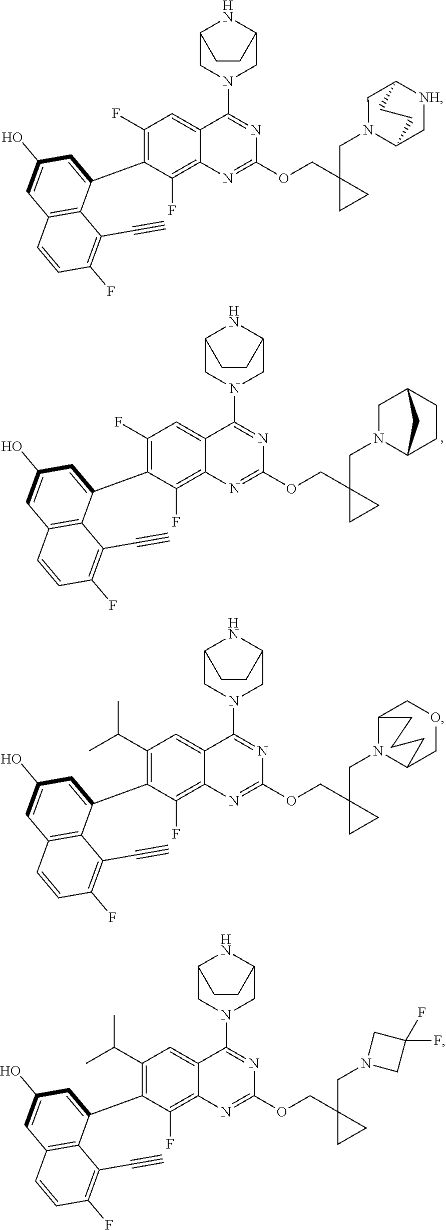

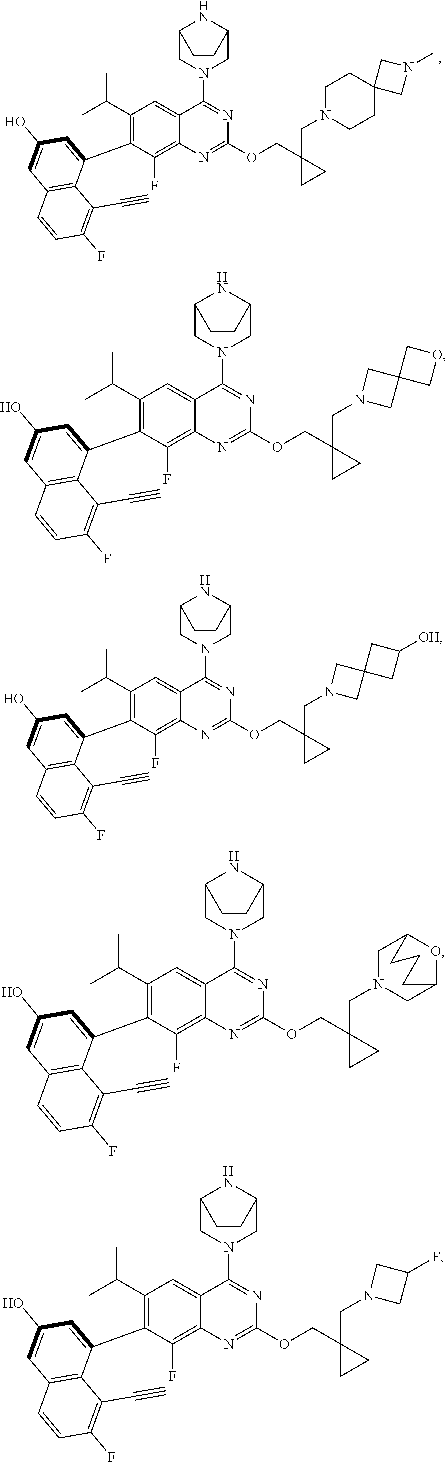

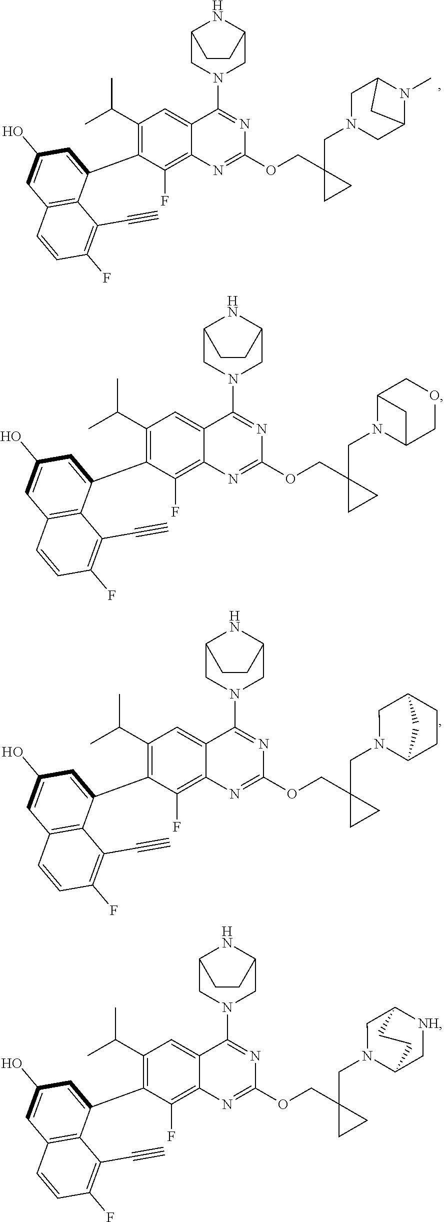

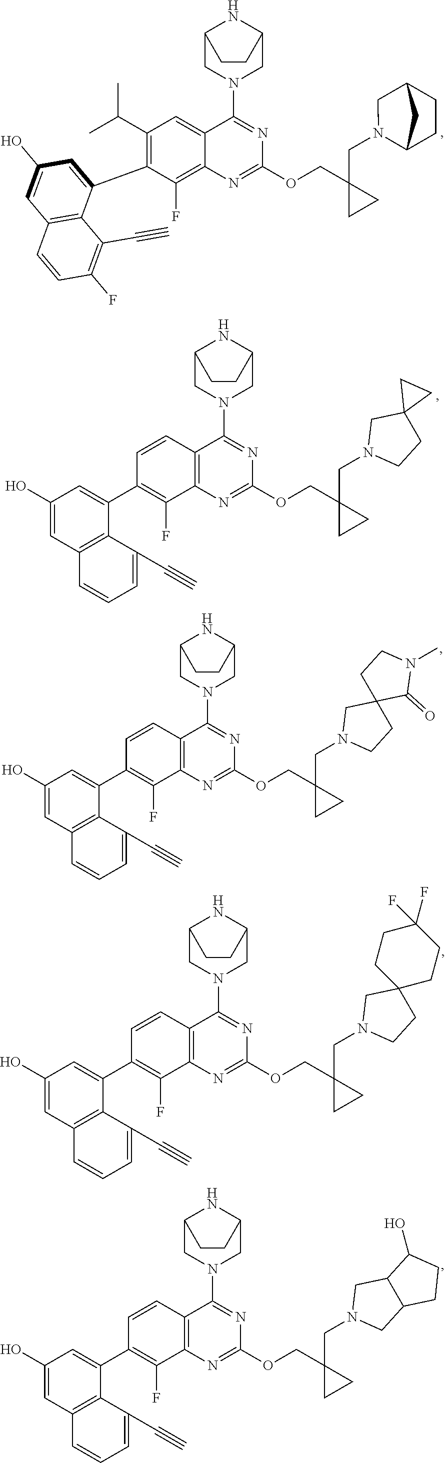

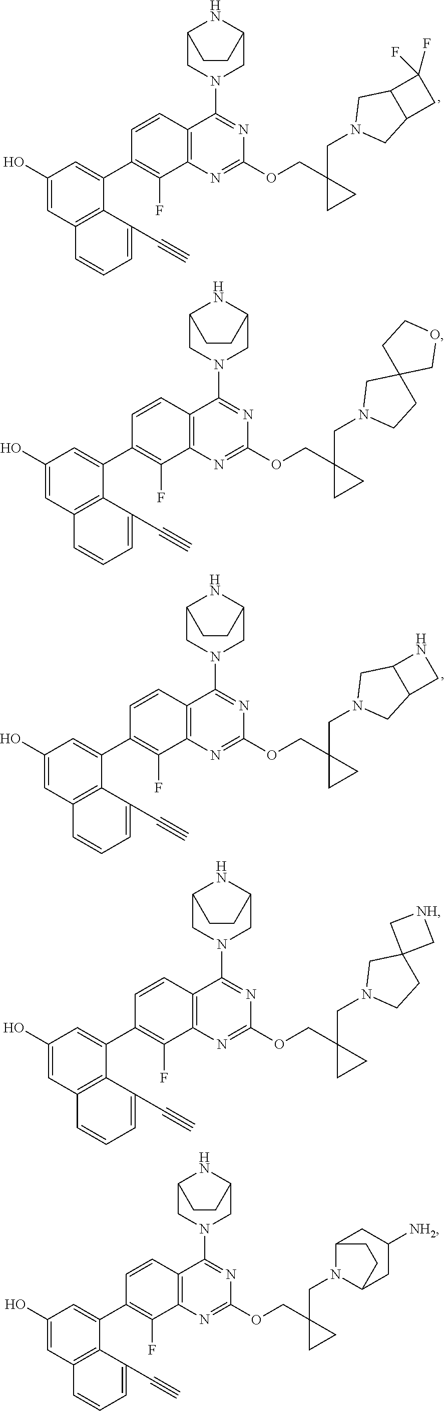

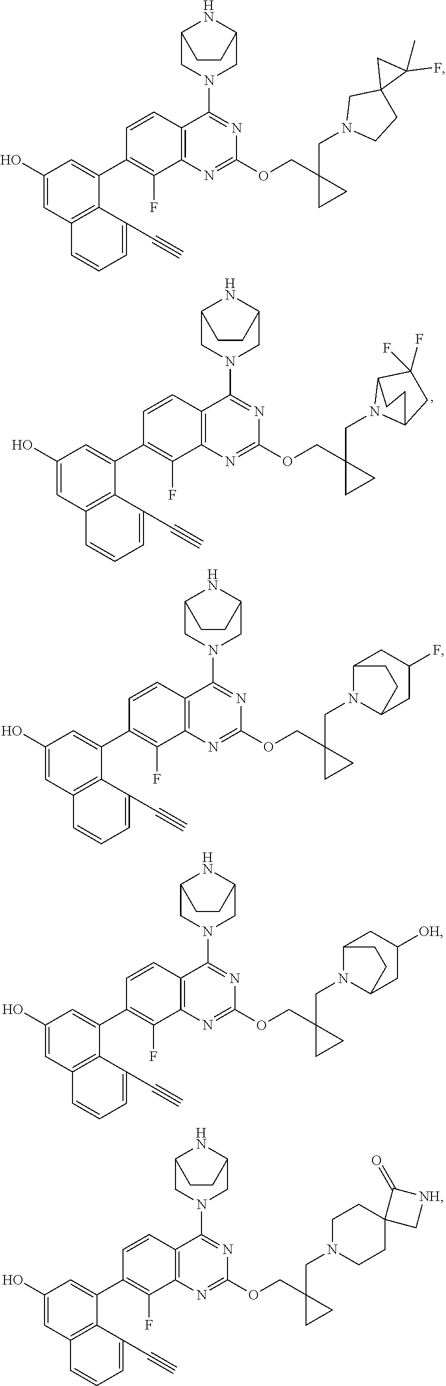

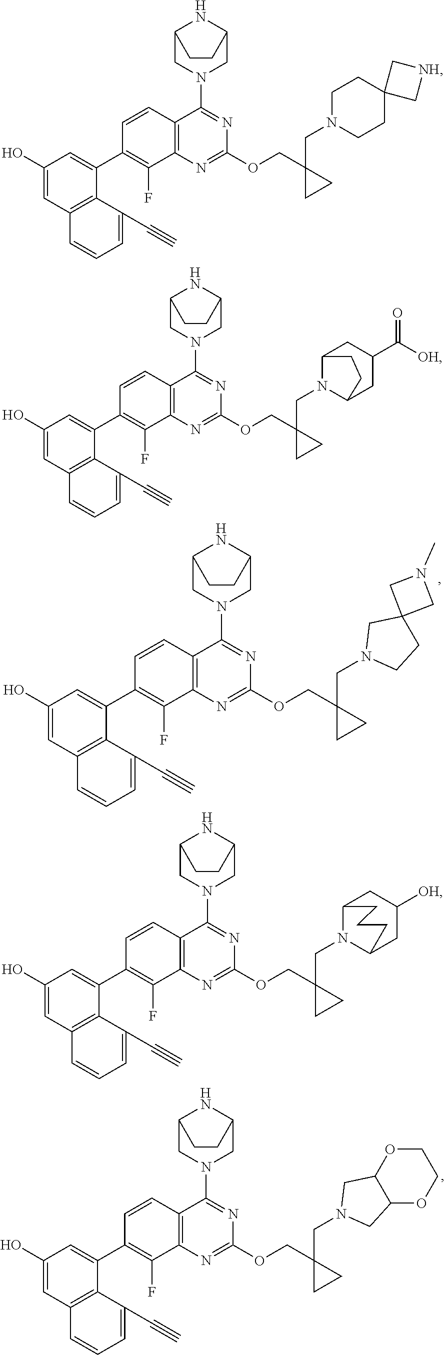

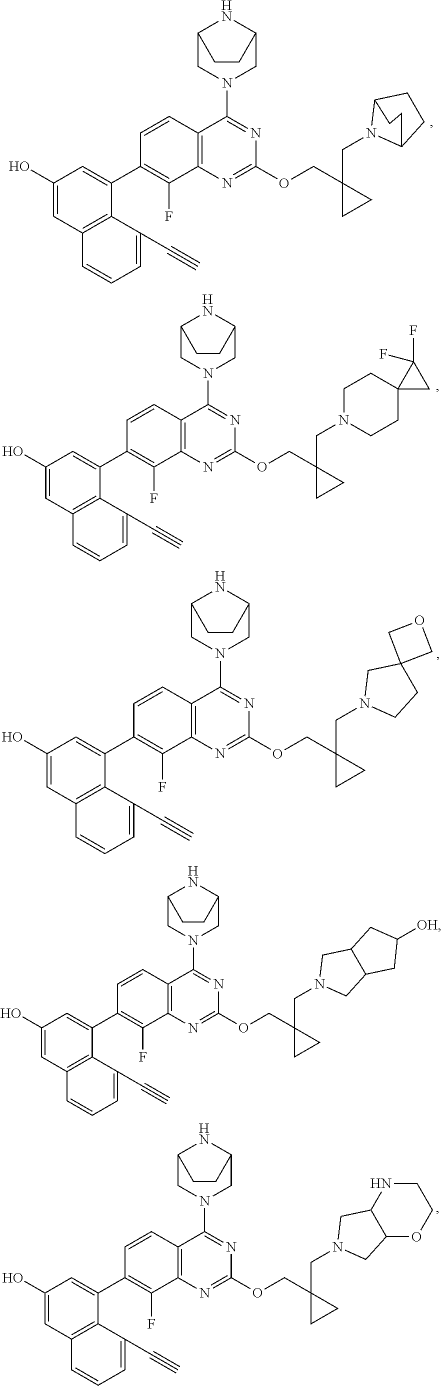 [SMILES: C#Cc1cccc2cc(O)cc(-c3ccc4c(N5CC6CCC(C5)N6)nc(OCC5(CN6C7CCC6CC7)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(COC7)C6)CC5)nc4c3F)c12 ; C#Cc1cccc2cc(O)cc(-c3ccc4c(N5CC6CCC(C5)N6)nc(OCC5(CN6CC7CC(O)CC7C6)CC5)nc4c3F)c12 ; C#Cc1cccc2cc(O)cc(-c3ccc4c(N5CC6CCC(C5)N6)nc(OCC5(CN6CC7NCCOC7C6)CC5)nc4c3F)c12]

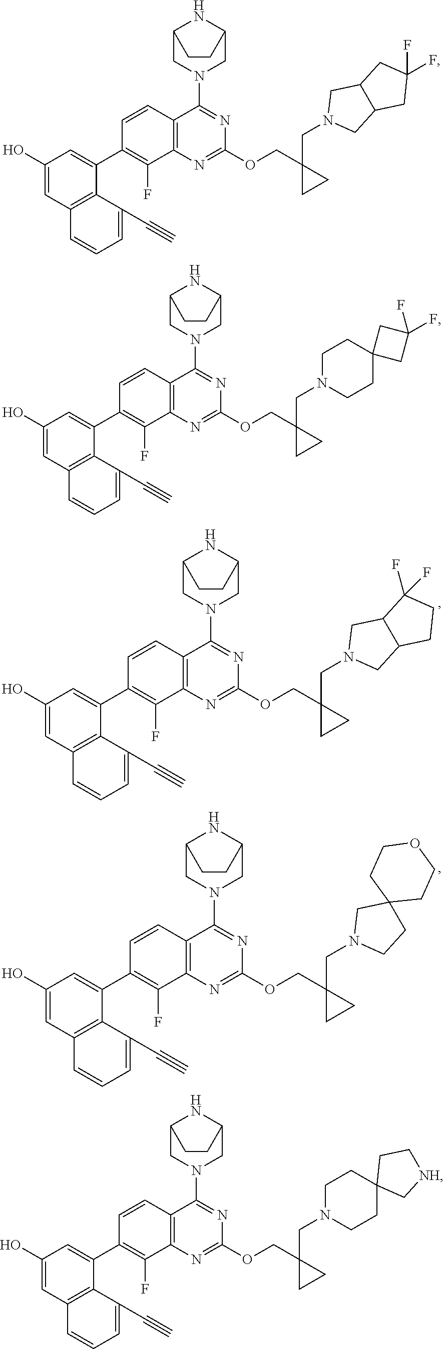 [SMILES: C#Cc1cccc2cc(O)cc(-c3ccc4c(N5CC6CCC(C5)N6)nc(OCC5(CN6CC7CC(F)(F)CC7C6)CC5)nc4c3F)c12 ; C#Cc1cccc2cc(O)cc(-c3ccc4c(N5CC6CCC(C5)N6)nc(OCC5(CN6CCC7(CC6)CC(F)(F)C7)CC5)nc4c3F)c12 ; C#Cc1cccc2cc(O)cc(-c3ccc4c(N5CC6CCC(C5)N6)nc(OCC5(CN6CC7CCC(F)(F)C7C6)CC5)nc4c3F)c12 ; C#Cc1cccc2cc(O)cc(-c3ccc4c(N5CC6CCC(C5)N6)nc(OCC5(CN6CCC7(CCOCC7)C6)CC5)nc4c3F)c12 ; C#Cc1cccc2cc(O)cc(-c3ccc4c(N5CC6CCC(C5)N6)nc(OCC5(CN6CCC7(CCNC7)CC6)CC5)nc4c3F)c12]

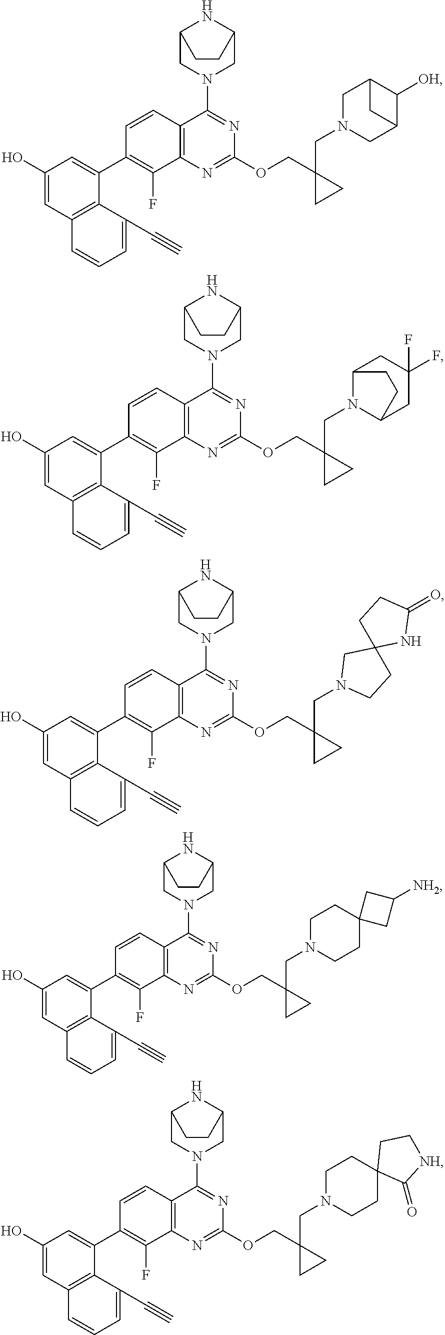

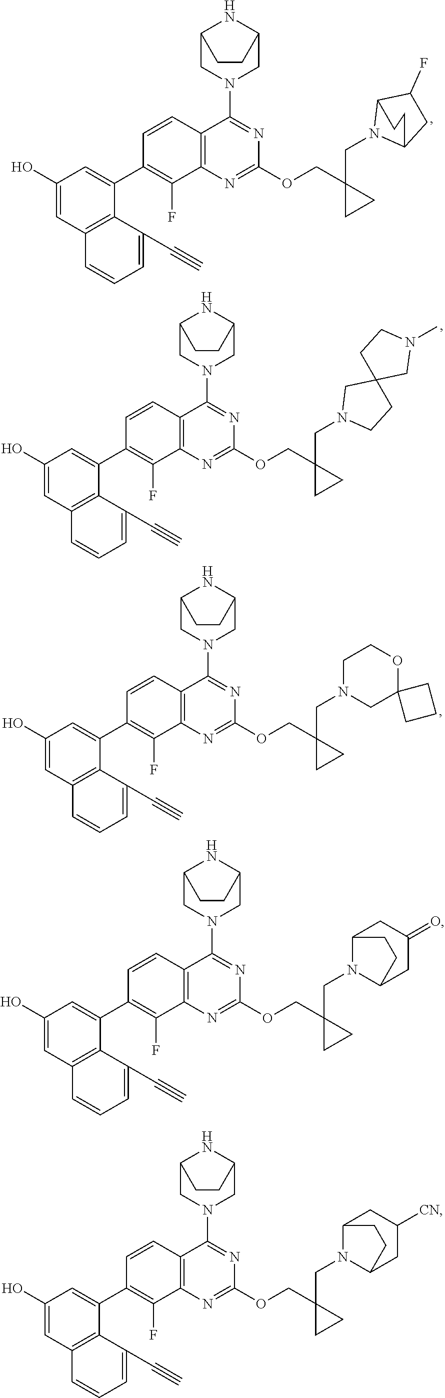

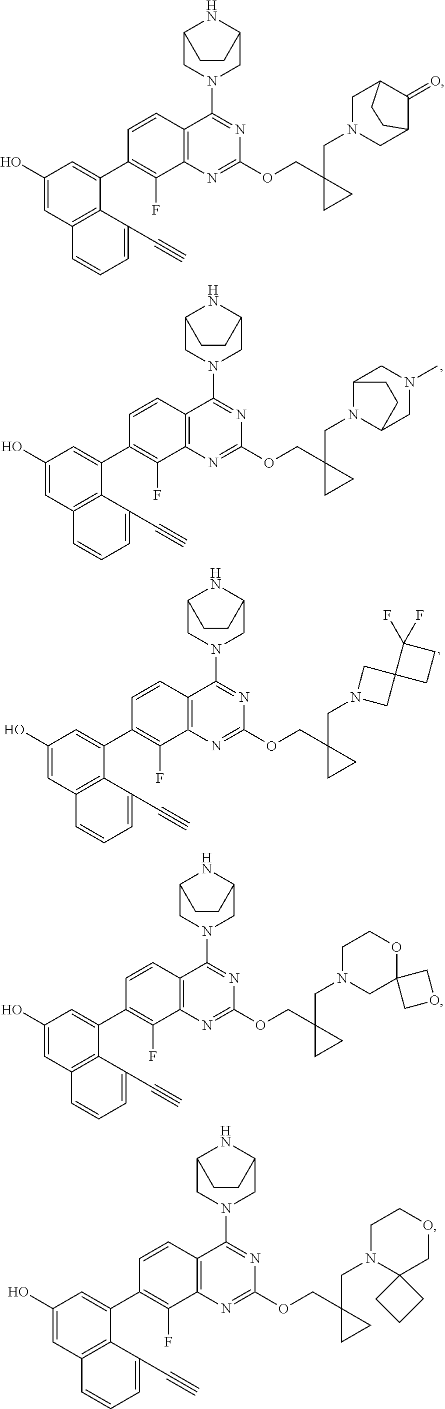 [SMILES: C#Cc1cccc2cc(O)cc(-c3ccc4c(N5CC6CCC(C5)N6)nc(OCC5(CN6CC7CCC(C6)C7=O)CC5)nc4c3F)c12 ; C#Cc1cccc2cc(O)cc(-c3ccc4c(N5CC6CCC(C5)N6)nc(OCC5(CN6C7CCC6CN(C)C7)CC5)nc4c3F)c12 ; C#Cc1cccc2cc(O)cc(-c3ccc4c(N5CC6CCC(C5)N6)nc(OCC5(CN6CC7(CCC7(F)F)C6)CC5)nc4c3F)c12 ; C#Cc1cccc2cc(O)cc(-c3ccc4c(N5CC6CCC(C5)N6)nc(OCC5(CN6CCOC7(COC7)C6)CC5)nc4c3F)c12 ; C#Cc1cccc2cc(O)cc(-c3ccc4c(N5CC6CCC(C5)N6)nc(OCC5(CN6CCOCC67CCC7)CC5)nc4c3F)c12]

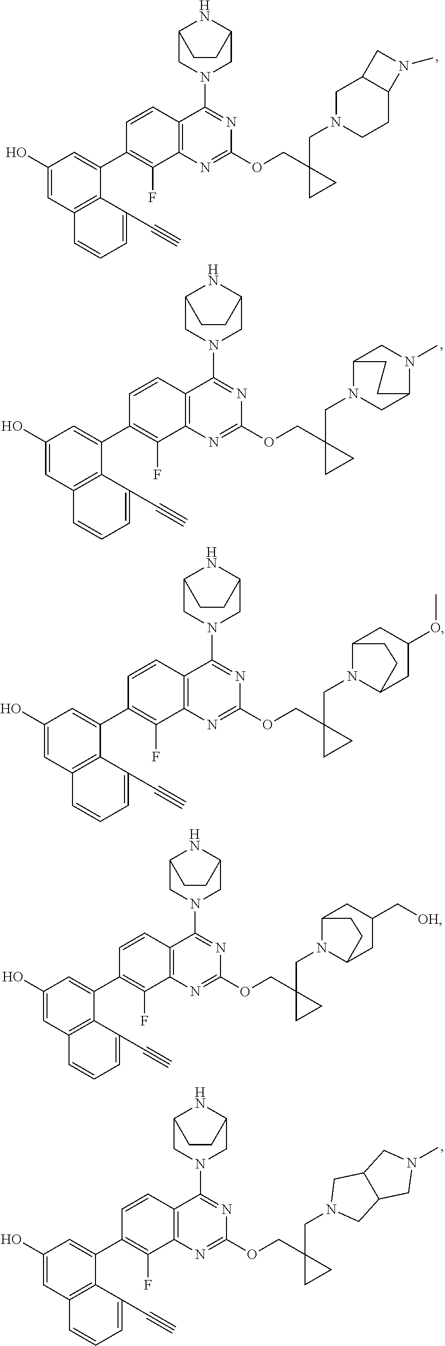

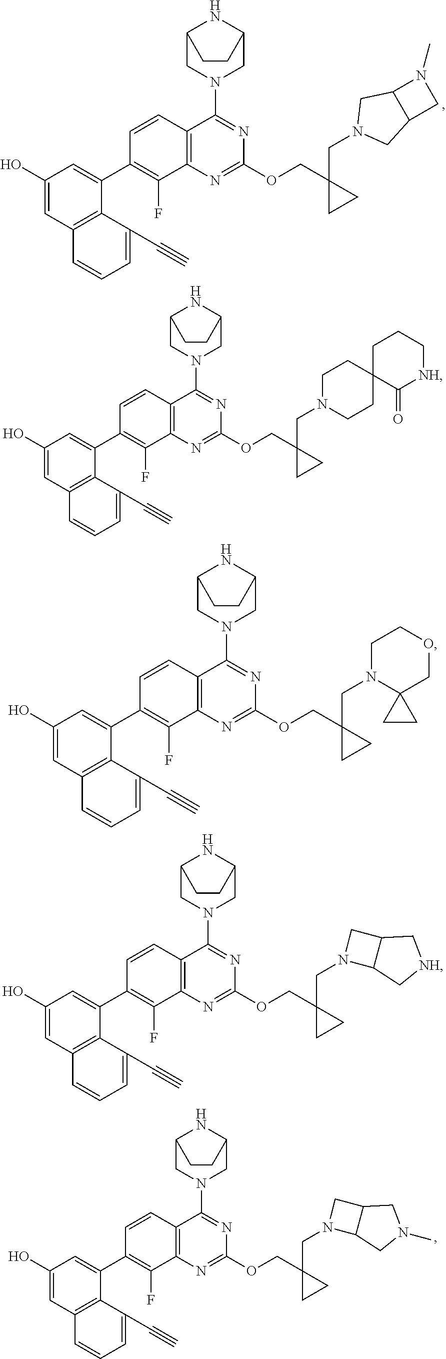

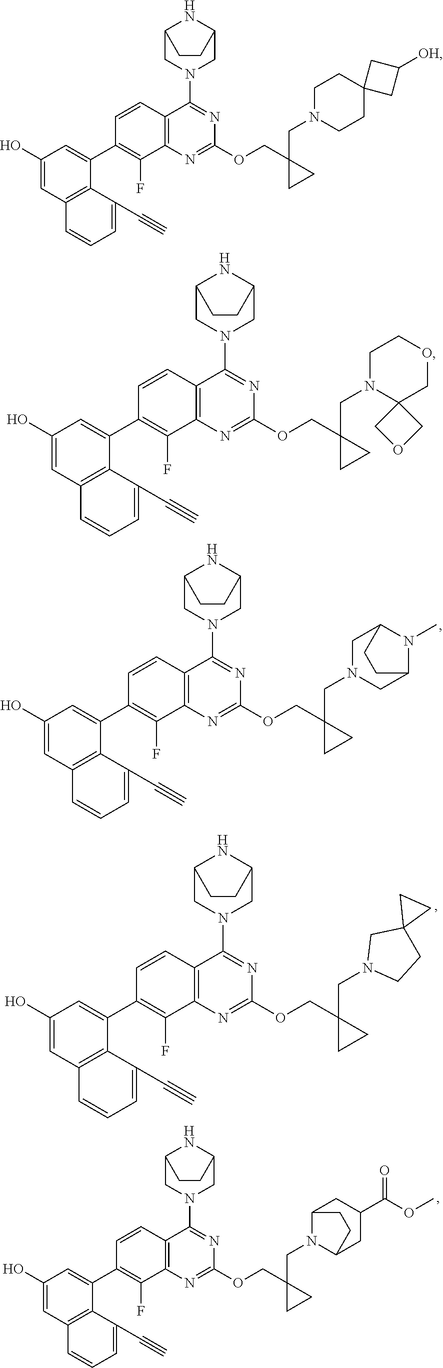

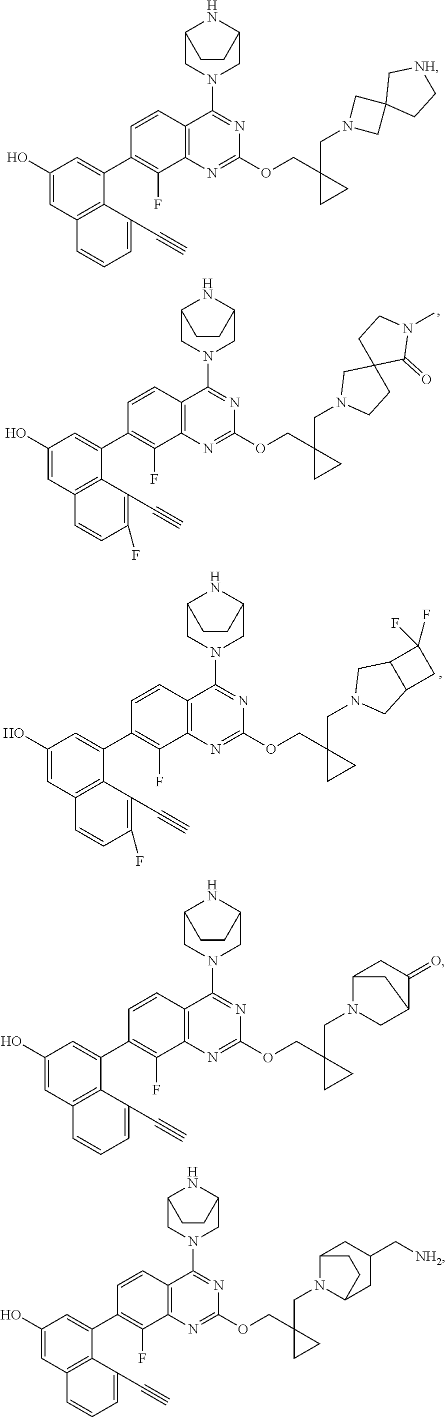 [SMILES: C#Cc1cccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CCC7(CCN(C)C7=O)C6)CC5)nc4c3F)c12 ; C#Cc1c(F)ccc2cc(O)cc(-c3ccc4c(N5CC6CCC(C5)N6)nc(OCC5(CN6CC7CC(F)(F)C7C6)CC5)nc4c3F)c12 ; C#Cc1cccc2cc(O)cc(-c3ccc4c(N5CC6CCC(C5)N6)nc(OCC5(CN6CC7CC6CC7=O)CC5)nc4c3F)c12 ; C#Cc1cccc2cc(O)cc(-c3ccc4c(N5CC6CCC(C5)N6)nc(OCC5(CN6C7CCC6CC(CN)C7)CC5)nc4c3F)c12]

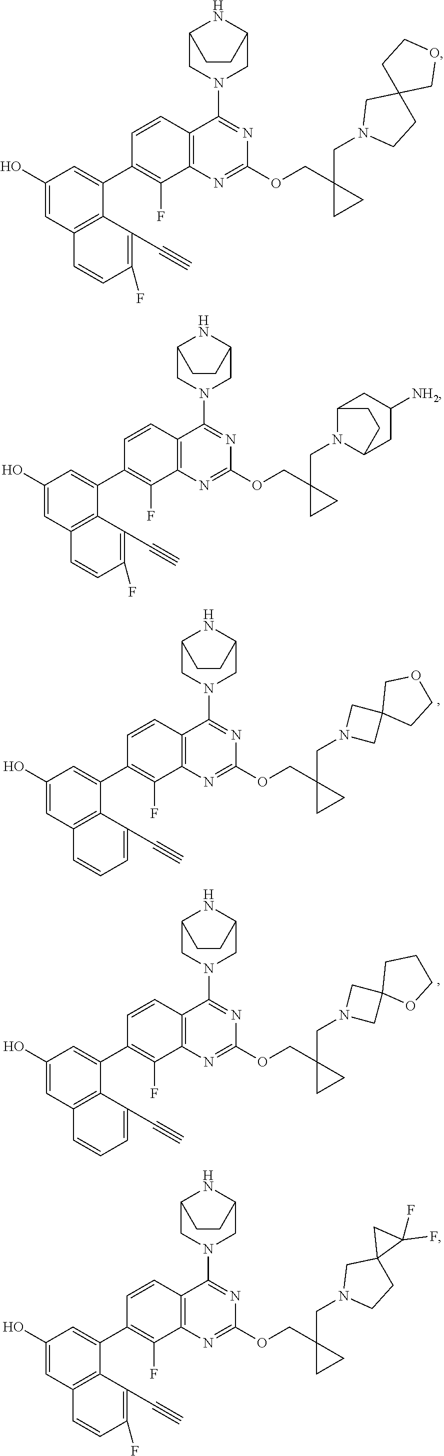 [SMILES: C#Cc1c(F)ccc2cc(O)cc(-c3ccc4c(N5CC6CCC(C5)N6)nc(OCC5(CN6CCC7(CCOC7)C6)CC5)nc4c3F)c12 ; C#Cc1c(F)ccc2cc(O)cc(-c3ccc4c(N5CC6CCC(C5)N6)nc(OCC5(CN6C7CCC6CC(N)C7)CC5)nc4c3F)c12 ; C#Cc1cccc2cc(O)cc(-c3ccc4c(N5CC6CCC(C5)N6)nc(OCC5(CN6CC7(CCOC7)C6)CC5)nc4c3F)c12 ; C#Cc1cccc2cc(O)cc(-c3ccc4c(N5CC6CCC(C5)N6)nc(OCC5(CN6CC7(CCCO7)C6)CC5)nc4c3F)c12 ; C#Cc1c(F)ccc2cc(O)cc(-c3ccc4c(N5CC6CCC(C5)N6)nc(OCC5(CN6CCC7(C6)CC7(F)F)CC5)nc4c3F)c12]

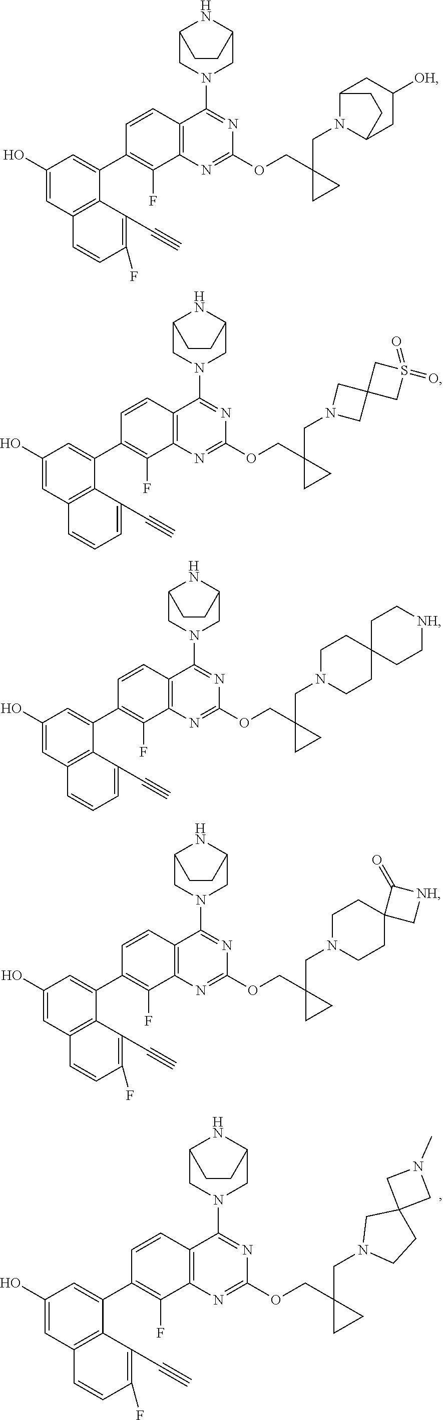 [SMILES: C#Cc1c(F)ccc2cc(O)cc(-c3ccc4c(N5CC6CCC(C5)N6)nc(OCC5(CN6C7CCC6CC(O)C7)CC5)nc4c3F)c12 ; C#Cc1cccc2cc(O)cc(-c3ccc4c(N5CC6CCC(C5)N6)nc(OCC5(CN6CC7(C6)CS(=O)(=O)C7)CC5)nc4c3F)c12 ; C#Cc1cccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CCC7(CC6)CNC7=O)CC5)nc4c3F)c12 ; C#Cc1c(F)ccc2cc(O)cc(-c3ccc4c(N5CC6CCC(C5)N6)nc(OCC5(CN6CCC7(CN(C)C7)C6)CC5)nc4c3F)c12]

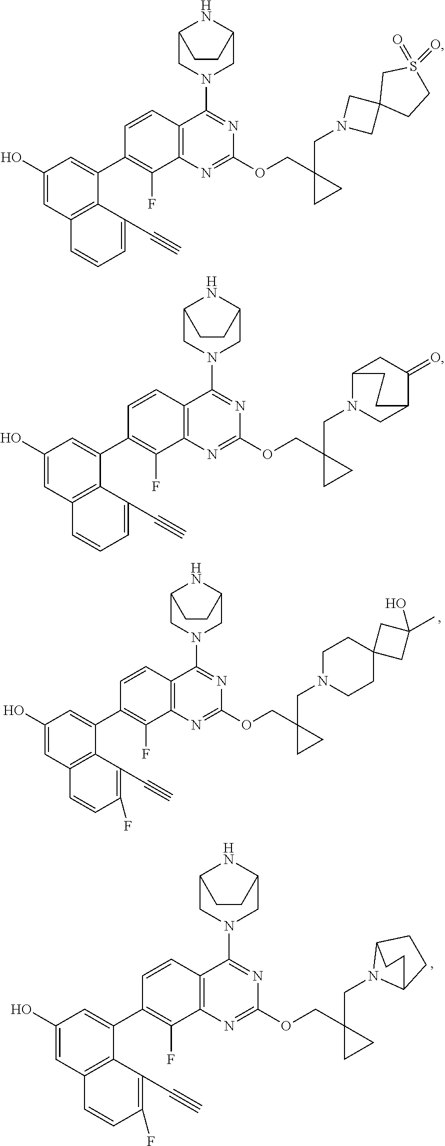 [SMILES: C#Cc1cccc2cc(O)cc(-c3ccc4c(N5CC6CCC(C5)N6)nc(OCC5(CN6CC7(CCS(=O)(=O)C7)C6)CC5)nc4c3F)c12 ; C#Cc1cccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6CCC7(CC6)CC(C)(O)C7)CC5)nc4c3F)c12 ; C#Cc1c(F)ccc2cc(O)cc(-c3ccc4c(N5CC6CCC(C5)N6)nc(OCC5(CN6C7CCC6CC7)CC5)nc4c3F)c12]

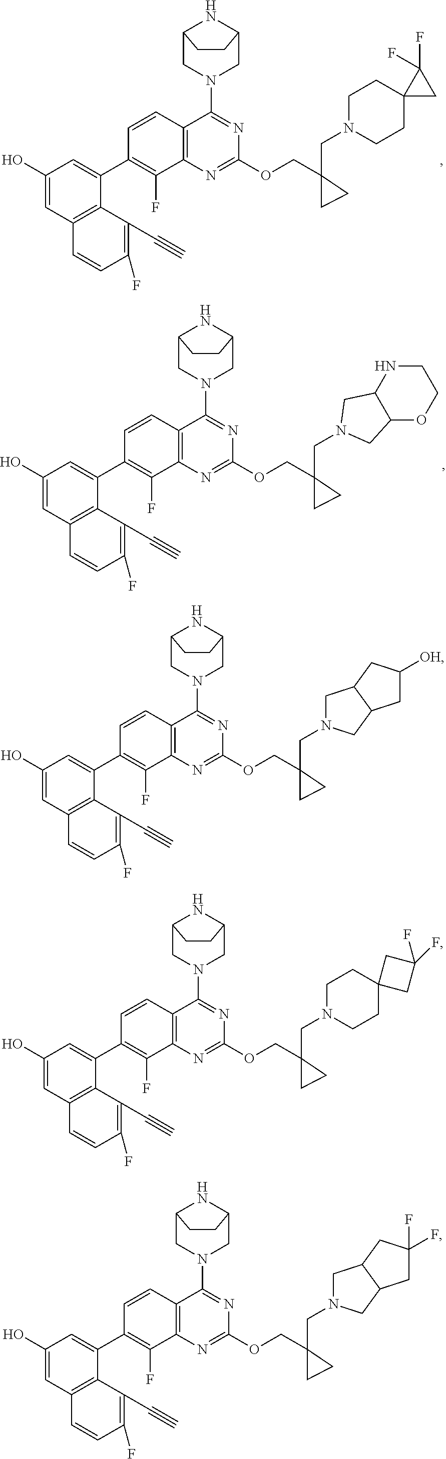 [SMILES: C#Cc1c(F)ccc2cc(O)cc(-c3ccc4c(N5CC6CCC(C5)N6)nc(OCC5(CN6CCC7(CC6)CC7(F)F)CC5)nc4c3F)c12 ; C#Cc1c(F)ccc2cc(O)cc(-c3ccc4c(N5CC6CCC(C5)N6)nc(OCC5(CN6CC7NCCOC7C6)CC5)nc4c3F)c12 ; C#Cc1c(F)ccc2cc(O)cc(-c3ccc4c(N5CC6CCC(C5)N6)nc(OCC5(CN6CC7CC(O)CC7C6)CC5)nc4c3F)c12 ; C#Cc1c(F)ccc2cc(O)cc(-c3ccc4c(N5CC6CCC(C5)N6)nc(OCC5(CN6CCC7(CC6)CC(F)(F)C7)CC5)nc4c3F)c12 ; C#Cc1c(F)ccc2cc(O)cc(-c3ccc4c(N5CC6CCC(C5)N6)nc(OCC5(CN6CC7CC(F)(F)CC7C6)CC5)nc4c3F)c12]

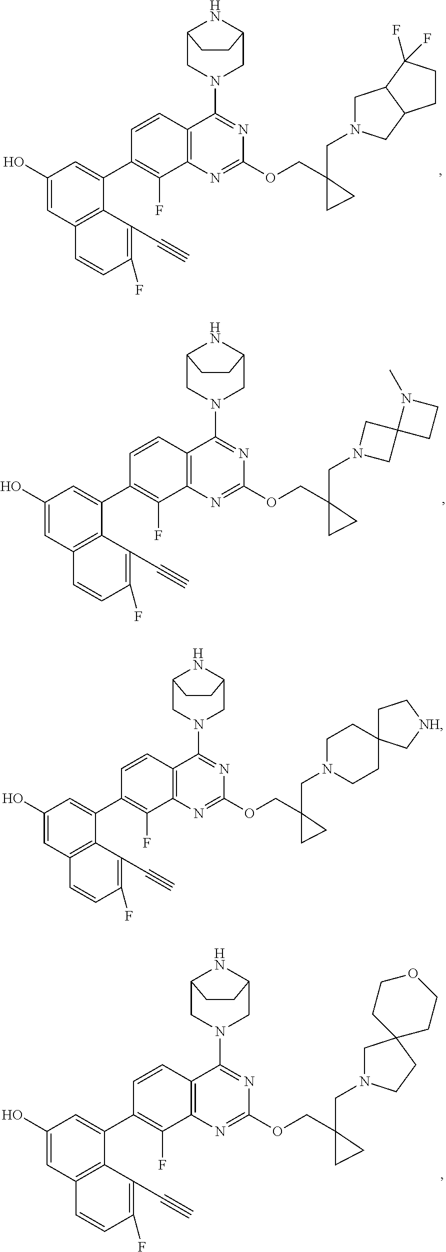 [SMILES: C#Cc1c(F)ccc2cc(O)cc(-c3ccc4c(N5CC6CCC(C5)N6)nc(OCC5(CN6CC7CCC(F)(F)C7C6)CC5)nc4c3F)c12 ; C#Cc1c(F)ccc2cc(O)cc(-c3ccc4c(N5CC6CCC(C5)N6)nc(OCC5(CN6CC7(CCN7C)C6)CC5)nc4c3F)c12 ; C#Cc1c(F)ccc2cc(O)cc(-c3ccc4c(N5CC6CCC(C5)N6)nc(OCC5(CN6CCC7(CCNC7)CC6)CC5)nc4c3F)c12 ; C#Cc1c(F)ccc2cc(O)cc(-c3ccc4c(N5CC6CCC(C5)N6)nc(OCC5(CN6CCC7(CCOCC7)C6)CC5)nc4c3F)c12]

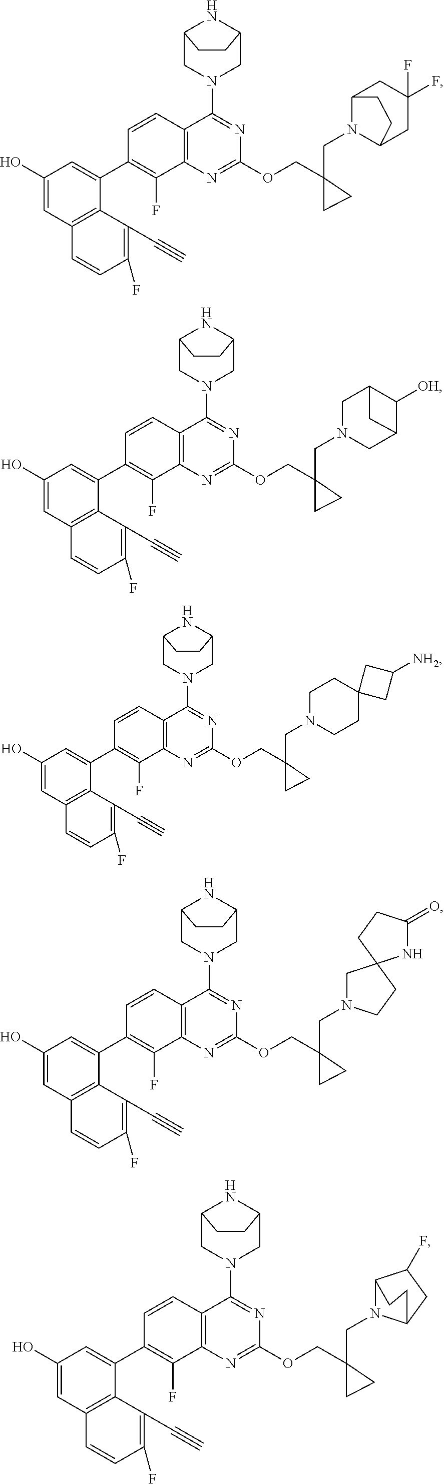 [SMILES: C#Cc1c(F)ccc2cc(O)cc(-c3ccc4c(N5CC6CCC(C5)N6)nc(OCC5(CN6C7CCC6CC(F)(F)C7)CC5)nc4c3F)c12 ; C#Cc1c(F)ccc2cc(O)cc(-c3ccc4c(N5CC6CCC(C5)N6)nc(OCC5(CN6CC7CC(C6)C7O)CC5)nc4c3F)c12 ; C#Cc1c(F)ccc2cc(O)cc(-c3ccc4c(N5CC6CCC(C5)N6)nc(OCC5(CN6CCC7(CC6)CC(N)C7)CC5)nc4c3F)c12 ; C#Cc1c(F)ccc2cc(O)cc(-c3ccc4c(N5CC6CCC(C5)N6)nc(OCC5(CN6CCC7(CCC(=O)N7)C6)CC5)nc4c3F)c12 ; C#Cc1c(F)ccc2cc(O)cc(-c3ccc4c(N5CC6CCC(C5)N6)nc(OCC5(CN6C7CCC6C(F)C7)CC5)nc4c3F)c12]

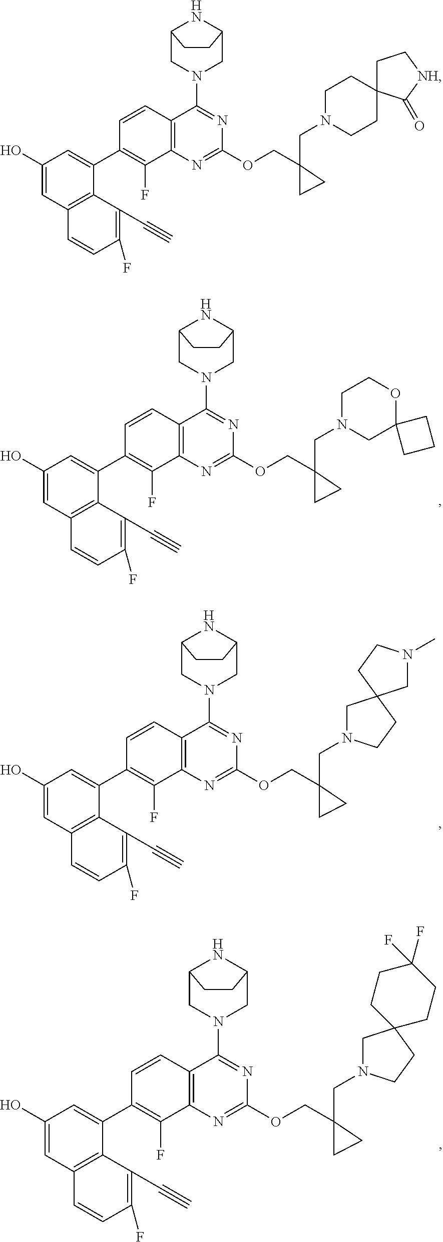 [SMILES: C#Cc1c(F)ccc2cc(O)cc(-c3ccc4c(N5CC6CCC(C5)N6)nc(OCC5(CN6CCC7(CCNC7=O)CC6)CC5)nc4c3F)c12 ; C#Cc1c(F)ccc2cc(O)cc(-c3ccc4c(N5CC6CCC(C5)N6)nc(OCC5(CN6CCOC7(CCC7)C6)CC5)nc4c3F)c12 ; C#Cc1c(F)ccc2cc(O)cc(-c3ccc4c(N5CC6CCC(C5)N6)nc(OCC5(CN6CCC7(CCN(C)C7)C6)CC5)nc4c3F)c12 ; C#Cc1c(F)ccc2cc(O)cc(-c3ccc4c(N5CC6CCC(C5)N6)nc(OCC5(CN6CCC7(CCC(F)(F)CC7)C6)CC5)nc4c3F)c12]

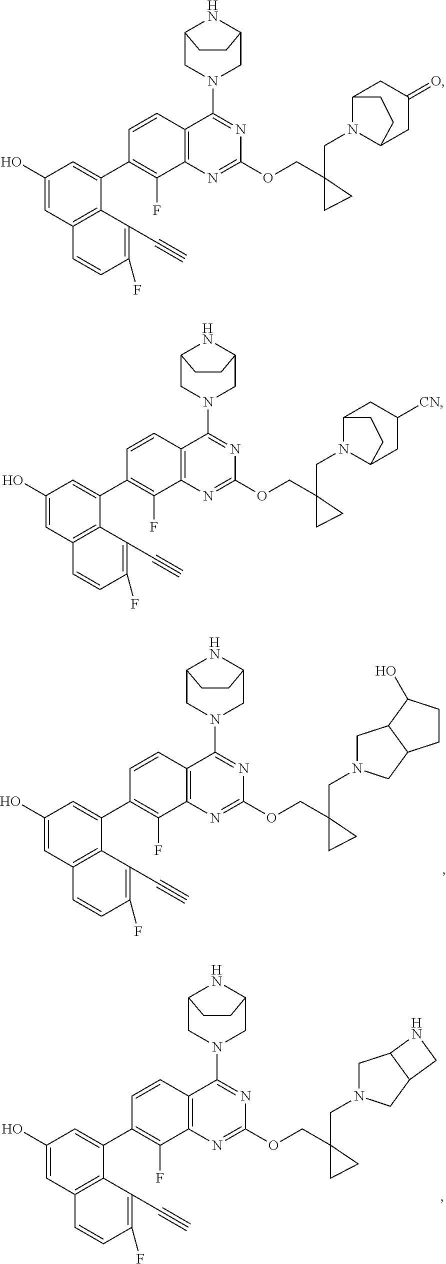 [SMILES: C#Cc1c(F)ccc2cc(O)cc(-c3ccc4c(N5CC6CCC(C5)N6)nc(OCC5(CN6C7CCC6CC(=O)C7)CC5)nc4c3F)c12 ; C#Cc1c(F)ccc2cc(O)cc(-c3ccc4c(N5CC6CCC(C5)N6)nc(OCC5(CN6C7CCC6CC(C#N)C7)CC5)nc4c3F)c12 ; C#Cc1c(F)ccc2cc(O)cc(-c3ccc4c(N5CC6CCC(C5)N6)nc(OCC5(CN6CC7CCC(O)C7C6)CC5)nc4c3F)c12 ; C#Cc1c(F)ccc2cc(O)cc(-c3ccc4c(N5CC6CCC(C5)N6)nc(OCC5(CN6CC7CNC7C6)CC5)nc4c3F)c12]

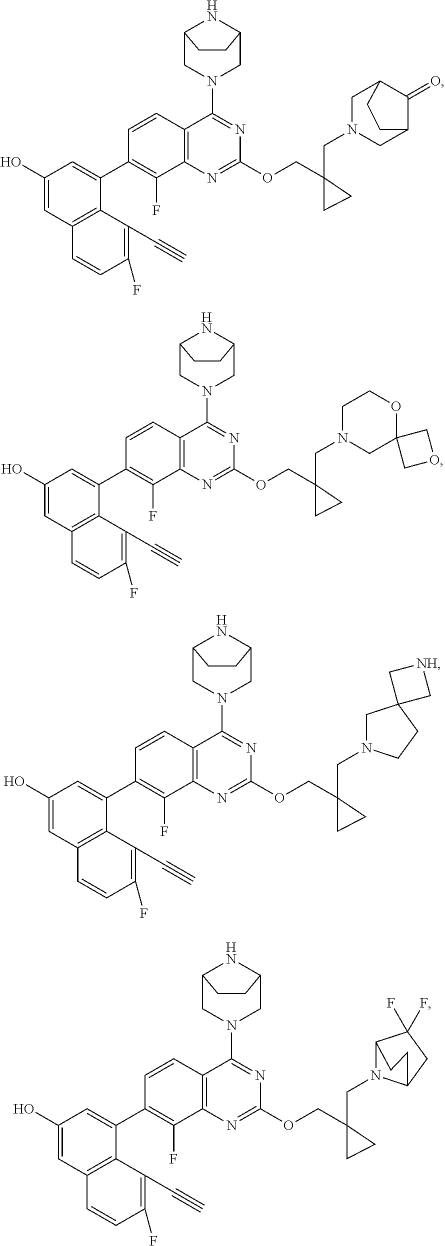 [SMILES: C#Cc1c(F)ccc2cc(O)cc(-c3ccc4c(N5CC6CCC(C5)N6)nc(OCC5(CN6CC7CCC(C6)C7=O)CC5)nc4c3F)c12 ; C#Cc1c(F)ccc2cc(O)cc(-c3ccc4c(N5CC6CCC(C5)N6)nc(OCC5(CN6CCOC7(COC7)C6)CC5)nc4c3F)c12 ; C#Cc1c(F)ccc2cc(O)cc(-c3ccc4c(N5CC6CCC(C5)N6)nc(OCC5(CN6CCC7(CNC7)C6)CC5)nc4c3F)c12 ; C#Cc1c(F)ccc2cc(O)cc(-c3ccc4c(N5CC6CCC(C5)N6)nc(OCC5(CN6C7CCC6C(F)(F)C7)CC5)nc4c3F)c12]

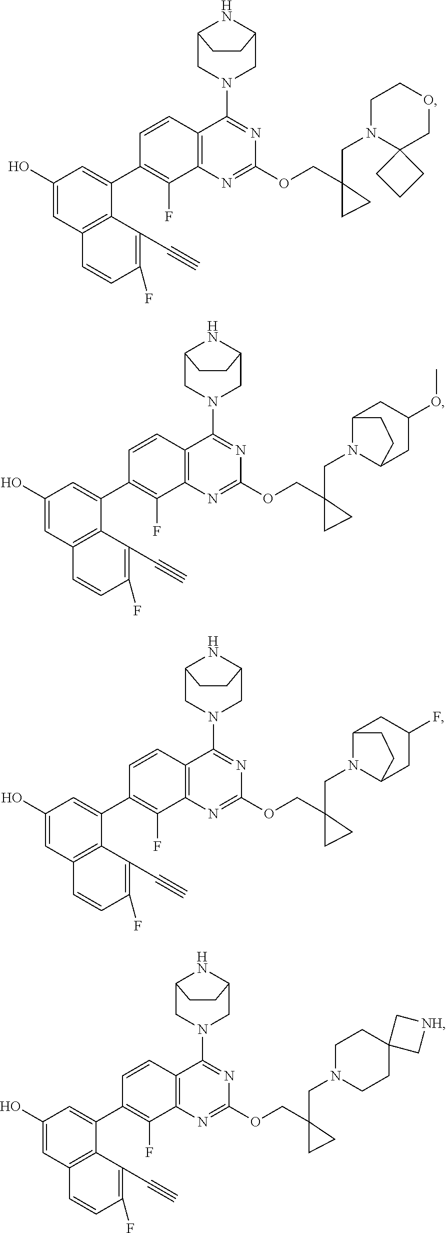 [SMILES: C#Cc1c(F)ccc2cc(O)cc(-c3ccc4c(N5CC6CCC(C5)N6)nc(OCC5(CN6CCOCC67CCC7)CC5)nc4c3F)c12 ; C#Cc1c(F)ccc2cc(O)cc(-c3ccc4c(N5CC6CCC(C5)N6)nc(OCC5(CN6C7CCC6CC(OC)C7)CC5)nc4c3F)c12 ; C#Cc1c(F)ccc2cc(O)cc(-c3ccc4c(N5CC6CCC(C5)N6)nc(OCC5(CN6C7CCC6CC(F)C7)CC5)nc4c3F)c12 ; C#Cc1c(F)ccc2cc(O)cc(-c3ccc4c(N5CC6CCC(C5)N6)nc(OCC5(CN6CCC7(CC6)CNC7)CC5)nc4c3F)c12]

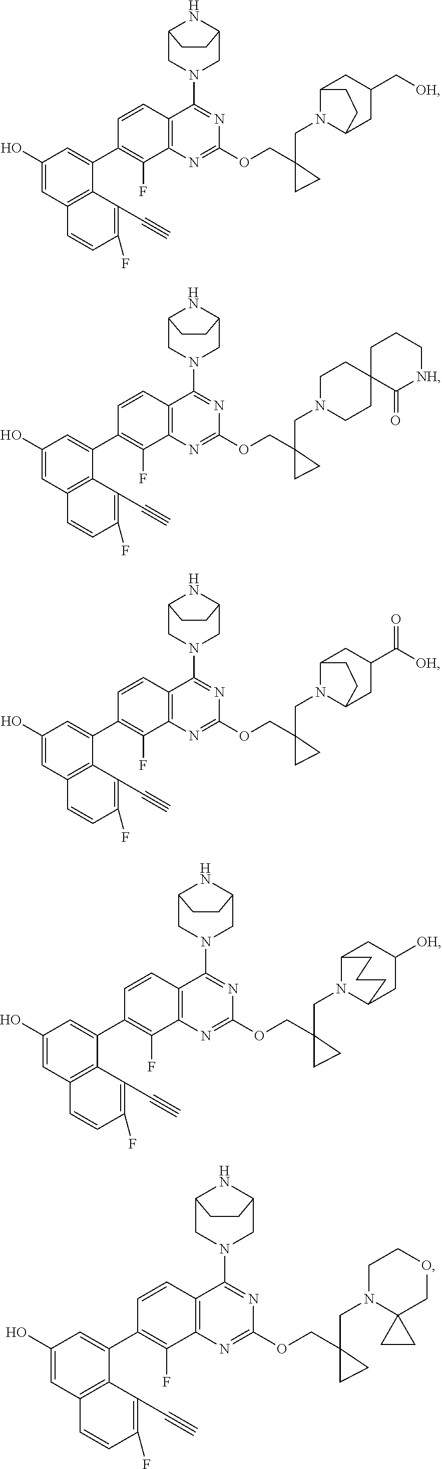 [SMILES: C#Cc1c(F)ccc2cc(O)cc(-c3ccc4c(N5CC6CCC(C5)N6)nc(OCC5(CN6C7CCC6CC(CO)C7)CC5)nc4c3F)c12 ; C#Cc1c(F)ccc2cc(O)cc(-c3ccc4c(N5CC6CCC(C5)N6)nc(OCC5(CN6CCC7(CCCNC7=O)CC6)CC5)nc4c3F)c12 ; C#Cc1c(F)ccc2cc(O)cc(-c3ccc4c(N5CC6CCC(C5)N6)nc(OCC5(CN6C7CCC6CC(C(=O)O)C7)CC5)nc4c3F)c12 ; C#Cc1c(F)ccc2cc(O)cc(-c3ccc4c(N5CC6CCC(C5)N6)nc(OCC5(CN6C7CCCC6CC(O)C7)CC5)nc4c3F)c12 ; C#Cc1c(F)ccc2cc(O)cc(-c3ccc4c(N5CC6CCC(C5)N6)nc(OCC5(CN6CCOCC67CC7)CC5)nc4c3F)c12]

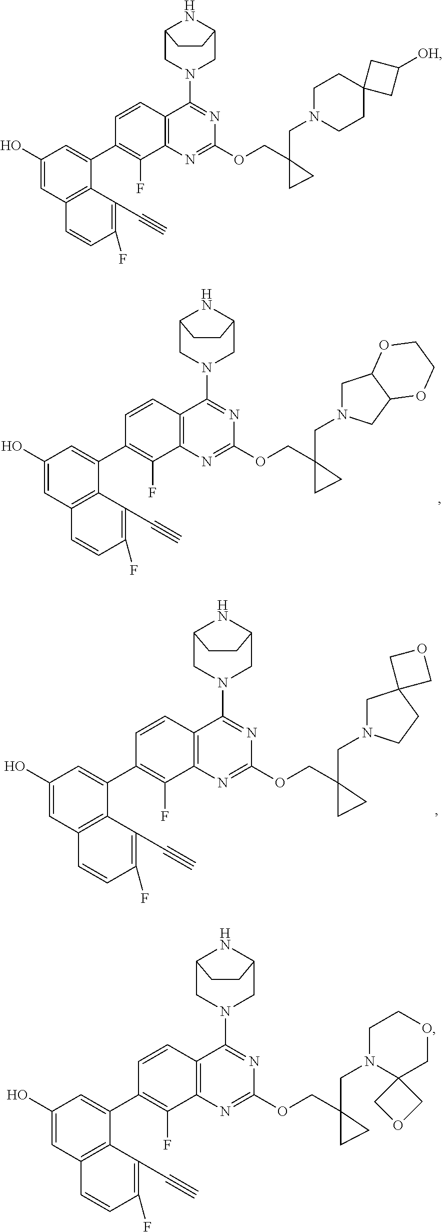 [SMILES: C#Cc1c(F)ccc2cc(O)cc(-c3ccc4c(N5CC6CCC(C5)N6)nc(OCC5(CN6CCC7(CC6)CC(O)C7)CC5)nc4c3F)c12 ; C#Cc1c(F)ccc2cc(O)cc(-c3ccc4c(N5CC6CCC(C5)N6)nc(OCC5(CN6CC7OCCOC7C6)CC5)nc4c3F)c12 ; C#Cc1c(F)ccc2cc(O)cc(-c3ccc4c(N5CC6CCC(C5)N6)nc(OCC5(CN6CCC7(COC7)C6)CC5)nc4c3F)c12 ; C#Cc1c(F)ccc2cc(O)cc(-c3ccc4c(N5CC6CCC(C5)N6)nc(OCC5(CN6CCOCC67COC7)CC5)nc4c3F)c12]

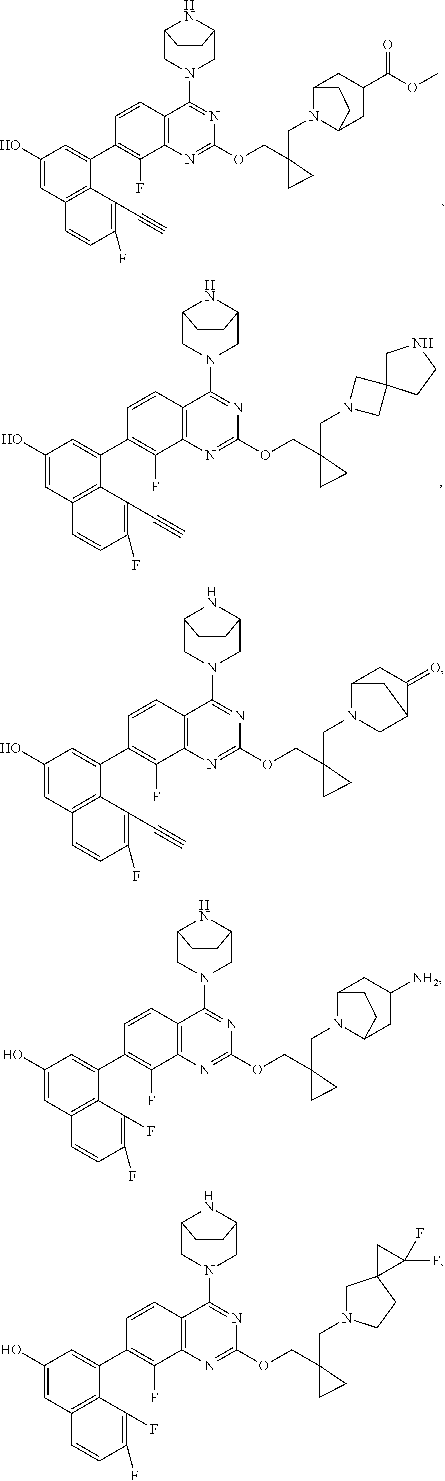 [SMILES: C#Cc1c(F)ccc2cc(O)cc(-c3ccc4c(N5CC6CCC(C5)N6)nc(OCC5(CN6C7CCC6CC(C(=O)OC)C7)CC5)nc4c3F)c12 ; C#Cc1c(F)ccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CC7CC6CC7=O)CC5)nc4c3F)c12 ; NC1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CCC6(C5)CC6(F)F)CC4)nc3c2F)c2c(F)c(F)ccc2c1]

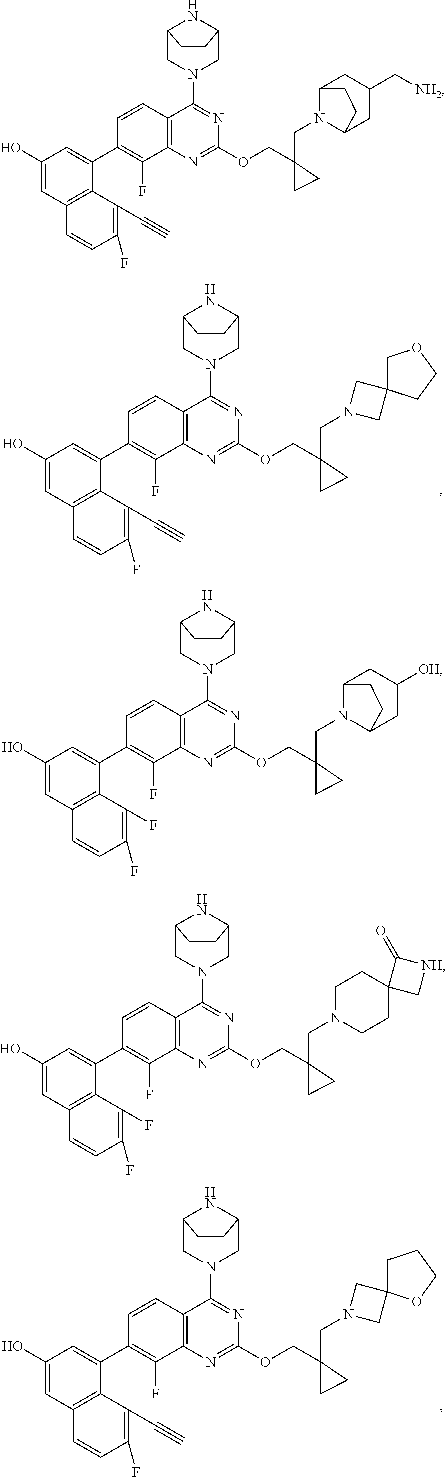 [SMILES: C#Cc1c(F)ccc2cc(O)cc(-c3ccc4c(N5CC6CCC(C5)N6)nc(OCC5(CN6C7CCC6CC(CN)C7)CC5)nc4c3F)c12 ; C#Cc1c(F)ccc2cc(O)cc(-c3ccc4c(N5CC6CCC(C5)N6)nc(OCC5(CN6CC7(CCOC7)C6)CC5)nc4c3F)c12 ; Oc1cc(-c2ccc3c(N4CC5CCC(C4)N5)nc(OCC4(CN5C6CCC5CC(O)C6)CC4)nc3c2F)c2c(F)c(F)ccc2c1 ; O=C1NCC12CCN(CC1(COc3nc(N4CC5CCC(C4)N5)c4ccc(-c5cc(O)cc6ccc(F)c(F)c56)c(F)c4n3)CC1)CC2 ; C#Cc1c(F)ccc2cc(O)cc(-c3ccc4c(N5CC6CCC(C5)N6)nc(OCC5(CN6CC7(CCCO7)C6)CC5)nc4c3F)c12]

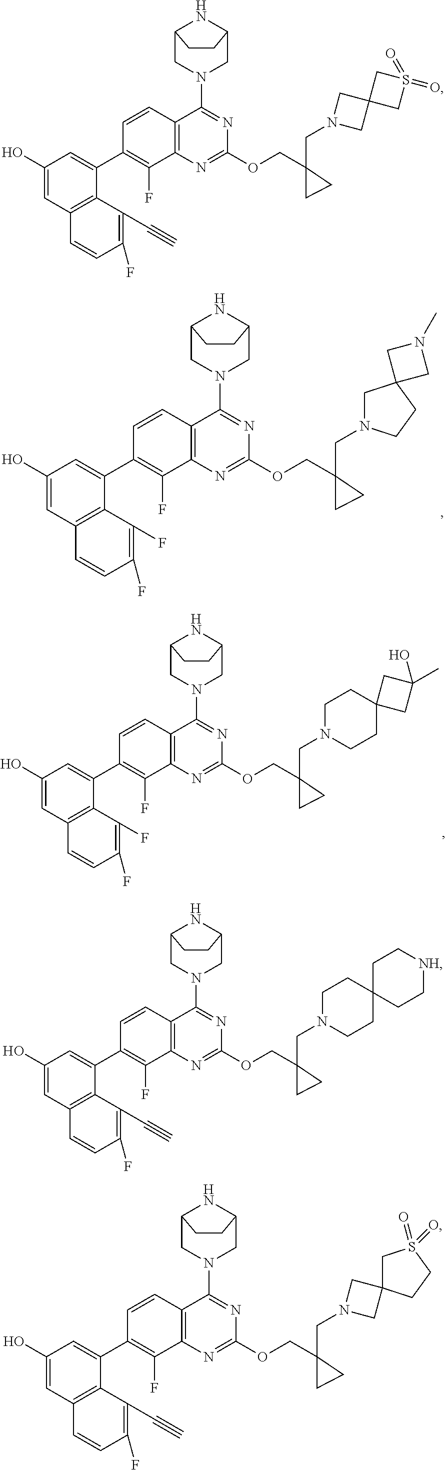 [SMILES: C#Cc1c(F)ccc2cc(O)cc(-c3ccc4c(N5CC6CCC(C5)N6)nc(OCC5(CN6CC7(C6)CS(=O)(=O)C7)CC5)nc4c3F)c12 ; CN1CC2(CCN(CC3(COc4nc(N5CC6CCC(C5)N6)c5ccc(-c6cc(O)cc7ccc(F)c(F)c67)c(F)c5n4)CC3)C2)C1 ; CC1(O)CC2(CCN(CC3(COc4nc(N5CC6CCC(C5)N6)c5ccc(-c6cc(O)cc7ccc(F)c(F)c67)c(F)c5n4)CC3)CC2)C1 ; C#Cc1c(F)ccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CC7(CCS(=O)(=O)C7)C6)CC5)nc4c3F)c12]

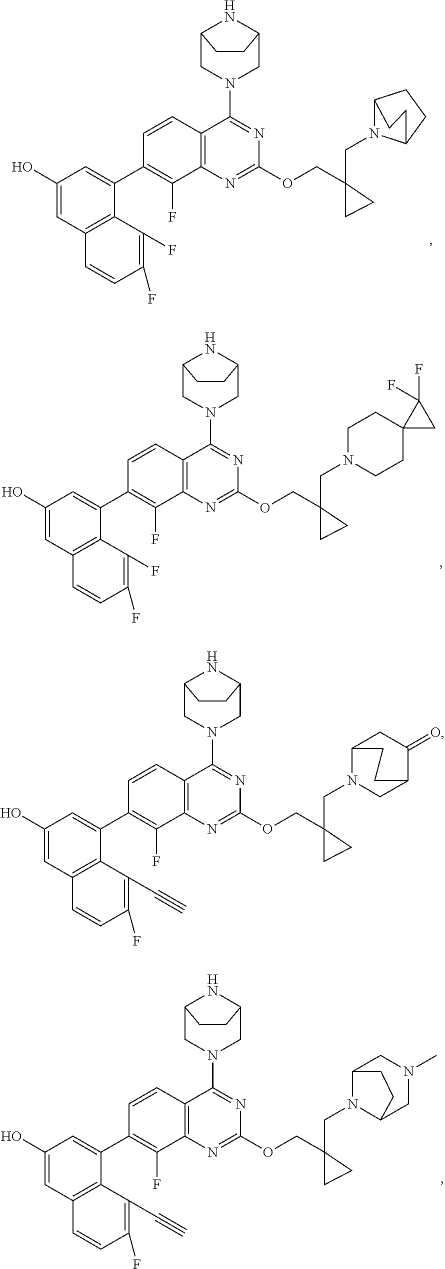 [SMILES: Oc1cc(-c2ccc3c(N4CC5CCC(C4)N5)nc(OCC4(CN5C6CCC5CC6)CC4)nc3c2F)c2c(F)c(F)ccc2c1 ; Oc1cc(-c2ccc3c(N4CC5CCC(C4)N5)nc(OCC4(CN5CCC6(CC5)CC6(F)F)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6C7CCC6CN(C)C7)CC5)nc4c3F)c12]

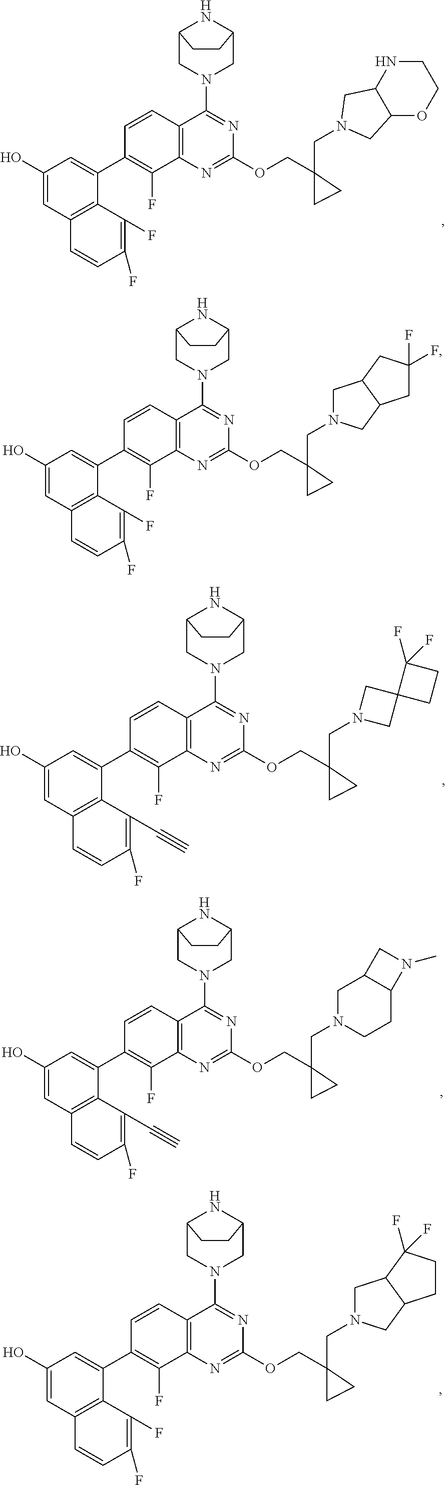 [SMILES: Oc1cc(-c2ccc3c(N4CC5CCC(C4)N5)nc(OCC4(CN5CC6NCCOC6C5)CC4)nc3c2F)c2c(F)c(F)ccc2c1 ; Oc1cc(-c2ccc3c(N4CC5CCC(C4)N5)nc(OCC4(CN5CC6CC(F)(F)CC6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7(CCC7(F)F)C6)CC5)nc4c3F)c12 ; C#Cc1c(F)ccc2cc(O)cc(-c3ccc4c(N5CC6CCC(C5)N6)nc(OCC5(CN6CCC7C(C6)CN7C)CC5)nc4c3F)c12 ; Oc1cc(-c2ccc3c(N4CC5CCC(C4)N5)nc(OCC4(CN5CC6CCC(F)(F)C6C5)CC4)nc3c2F)c2c(F)c(F)ccc2c1]

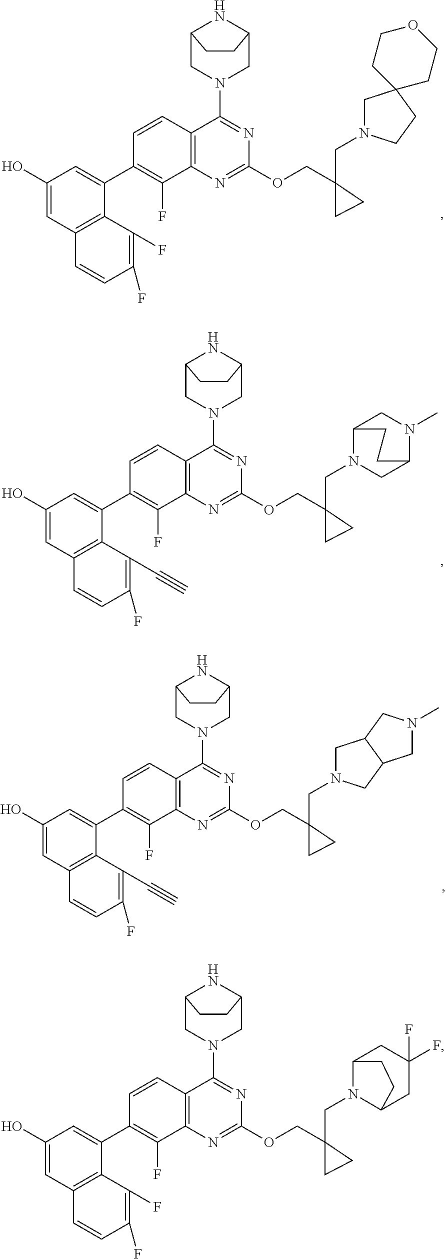 [SMILES: Oc1cc(-c2ccc3c(N4CC5CCC(C4)N5)nc(OCC4(CN5CCC6(CCOCC6)C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7CCC6CN7C)CC5)nc4c3F)c12 ; C#Cc1c(F)ccc2cc(O)cc(-c3ccc4c(N5CC6CCC(C5)N6)nc(OCC5(CN6CC7CN(C)CC7C6)CC5)nc4c3F)c12 ; Oc1cc(-c2ccc3c(N4CC5CCC(C4)N5)nc(OCC4(CN5C6CCC5CC(F)(F)C6)CC4)nc3c2F)c2c(F)c(F)ccc2c1]

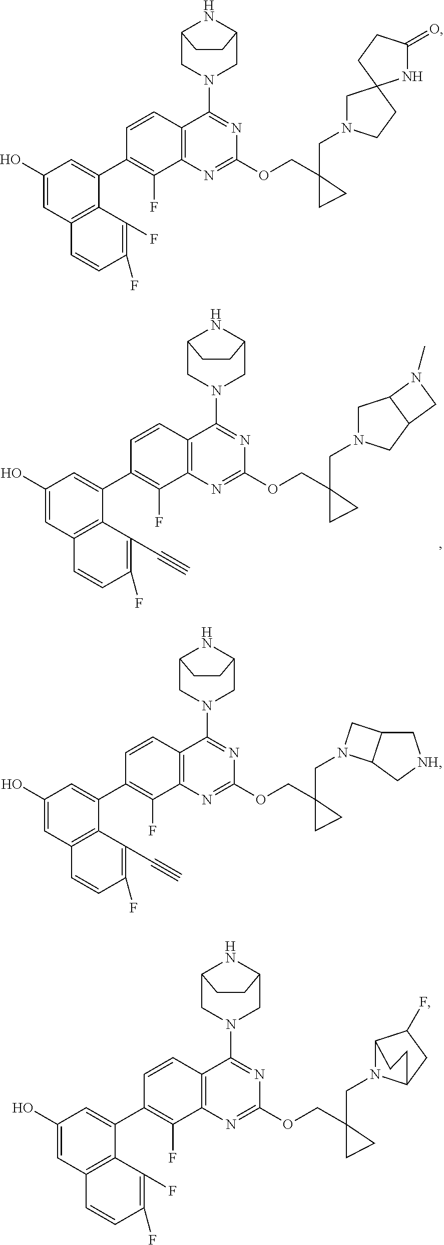 [SMILES: O=C1CCC2(CCN(CC3(COc4nc(N5CC6CCC(C5)N6)c5ccc(-c6cc(O)cc7ccc(F)c(F)c67)c(F)c5n4)CC3)C2)N1 ; C#Cc1c(F)ccc2cc(O)cc(-c3ccc4c(N5CC6CCC(C5)N6)nc(OCC5(CN6CC7CN(C)C7C6)CC5)nc4c3F)c12 ; C#Cc1c(F)ccc2cc(O)cc(-c3ccc4c(N5CC6CCC(C5)N6)nc(OCC5(CN6CC7CNCC76)CC5)nc4c3F)c12 ; Oc1cc(-c2ccc3c(N4CC5CCC(C4)N5)nc(OCC4(CN5C6CCC5C(F)C6)CC4)nc3c2F)c2c(F)c(F)ccc2c1]

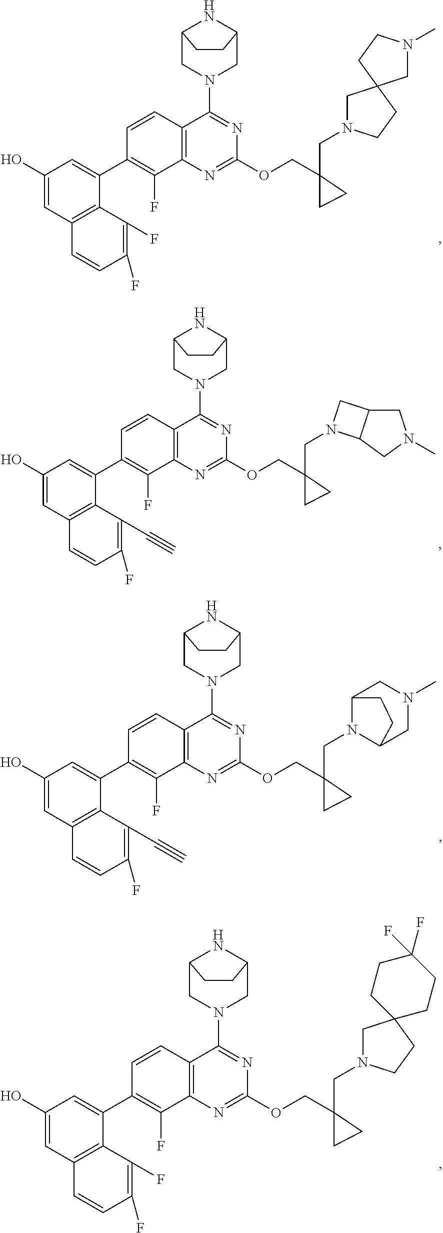 [SMILES: CN1CCC2(CCN(CC3(COc4nc(N5CC6CCC(C5)N6)c5ccc(-c6cc(O)cc7ccc(F)c(F)c67)c(F)c5n4)CC3)C2)C1 ; C#Cc1c(F)ccc2cc(O)cc(-c3ccc4c(N5CC6CCC(C5)N6)nc(OCC5(CN6CC7CN(C)CC76)CC5)nc4c3F)c12 ; C#Cc1c(F)ccc2cc(O)cc(-c3ccc4c(N5CC6CCC(C5)N6)nc(OCC5(CN6C7CCC6CN(C)C7)CC5)nc4c3F)c12 ; Oc1cc(-c2ccc3c(N4CC5CCC(C4)N5)nc(OCC4(CN5CCC6(CCC(F)(F)CC6)C5)CC4)nc3c2F)c2c(F)c(F)ccc2c1]

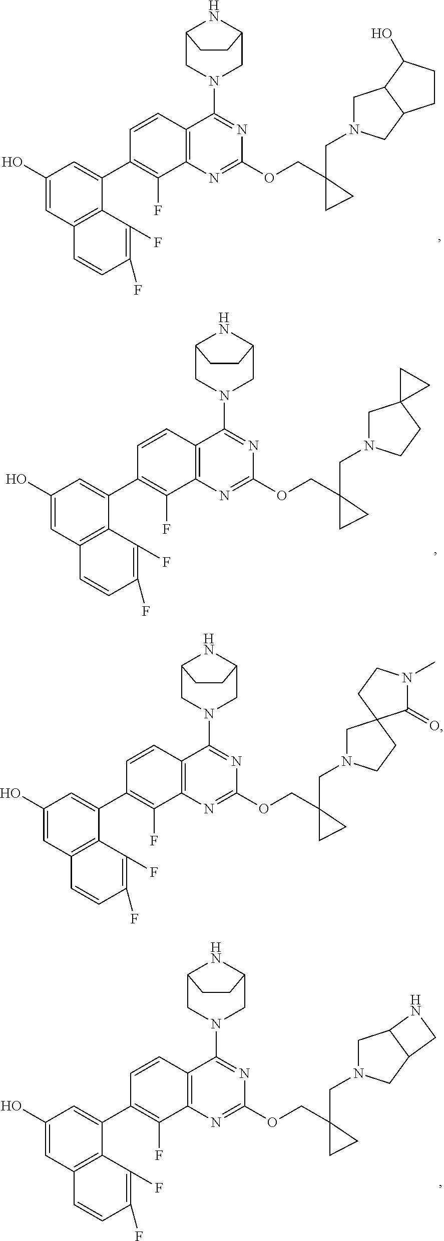 [SMILES: Oc1cc(-c2ccc3c(N4CC5CCC(C4)N5)nc(OCC4(CN5CC6CCC(O)C6C5)CC4)nc3c2F)c2c(F)c(F)ccc2c1 ; Oc1cc(-c2ccc3c(N4CC5CCC(C4)N5)nc(OCC4(CN5CCC6(CC6)C5)CC4)nc3c2F)c2c(F)c(F)ccc2c1 ; CN1CCC2(CCN(CC3(COc4nc(N5CC6CCC(C5)N6)c5ccc(-c6cc(O)cc7ccc(F)c(F)c67)c(F)c5n4)CC3)C2)C1=O ; Oc1cc(-c2ccc3c(N4CC5CCC(C4)N5)nc(OCC4(CN5CC6CNC6C5)CC4)nc3c2F)c2c(F)c(F)ccc2c1]

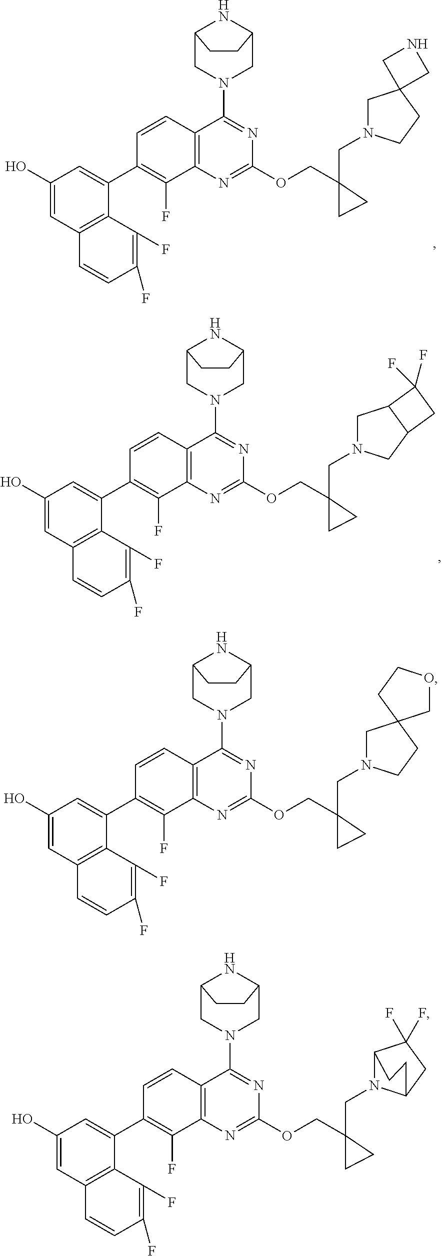 [SMILES: Oc1cc(-c2ccc3c(N4CC5CCC(C4)N5)nc(OCC4(CN5CCC6(CNC6)C5)CC4)nc3c2F)c2c(F)c(F)ccc2c1 ; Oc1cc(-c2ccc3c(N4CC5CCC(C4)N5)nc(OCC4(CN5CC6CC(F)(F)C6C5)CC4)nc3c2F)c2c(F)c(F)ccc2c1 ; Oc1cc(-c2ccc3c(N4CC5CCC(C4)N5)nc(OCC4(CN5CCC6(CCOC6)C5)CC4)nc3c2F)c2c(F)c(F)ccc2c1 ; Oc1cc(-c2ccc3c(N4CC5CCC(C4)N5)nc(OCC4(CN5C6CCC5C(F)(F)C6)CC4)nc3c2F)c2c(F)c(F)ccc2c1]

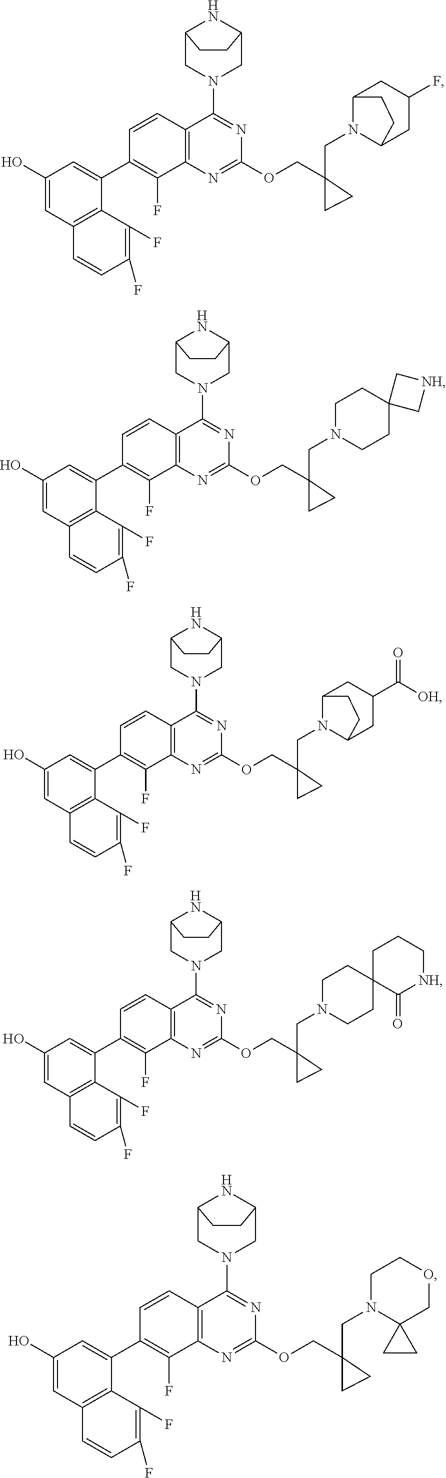 [SMILES: Oc1cc(-c2ccc3c(N4CC5CCC(C4)N5)nc(OCC4(CN5C6CCC5CC(F)C6)CC4)nc3c2F)c2c(F)c(F)ccc2c1 ; Oc1cc(-c2ccc3c(N4CC5CCC(C4)N5)nc(OCC4(CN5CCC6(CC5)CNC6)CC4)nc3c2F)c2c(F)c(F)ccc2c1 ; O=C(O)C1CC2CCC(C1)N2CC1(COc2nc(N3CC4CCC(C3)N4)c3ccc(-c4cc(O)cc5ccc(F)c(F)c45)c(F)c3n2)CC1 ; O=C1NCCCC12CCN(CC1(COc3nc(N4CC5CCC(C4)N5)c4ccc(-c5cc(O)cc6ccc(F)c(F)c56)c(F)c4n3)CC1)CC2 ; Oc1cc(-c2ccc3c(N4CC5CCC(C4)N5)nc(OCC4(CN5CCOCC56CC6)CC4)nc3c2F)c2c(F)c(F)ccc2c1]

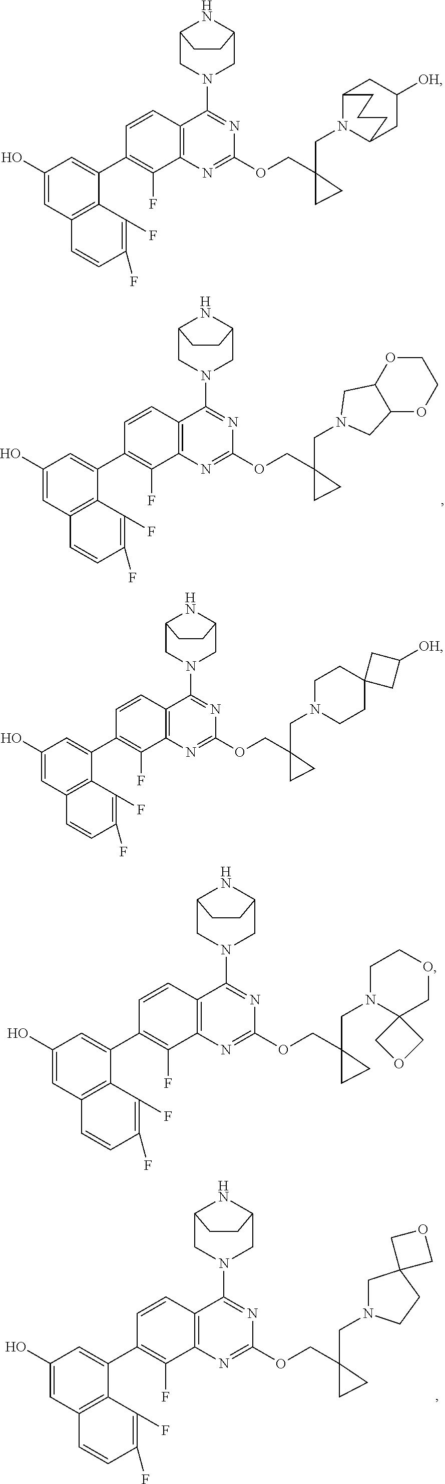 [SMILES: Oc1cc(-c2ccc3c(N4CC5CCC(C4)N5)nc(OCC4(CN5C6CCCC5CC(O)C6)CC4)nc3c2F)c2c(F)c(F)ccc2c1 ; Oc1cc(-c2ccc3c(N4CC5CCC(C4)N5)nc(OCC4(CN5CC6OCCOC6C5)CC4)nc3c2F)c2c(F)c(F)ccc2c1 ; Oc1cc(-c2ccc3c(N4CC5CCC(C4)N5)nc(OCC4(CN5CCC6(CC5)CC(O)C6)CC4)nc3c2F)c2c(F)c(F)ccc2c1 ; Oc1cc(-c2ccc3c(N4CC5CCC(C4)N5)nc(OCC4(CN5CCOCC56COC6)CC4)nc3c2F)c2c(F)c(F)ccc2c1 ; Oc1cc(-c2ccc3c(N4CC5CCC(C4)N5)nc(OCC4(CN5CCC6(COC6)C5)CC4)nc3c2F)c2c(F)c(F)ccc2c1]

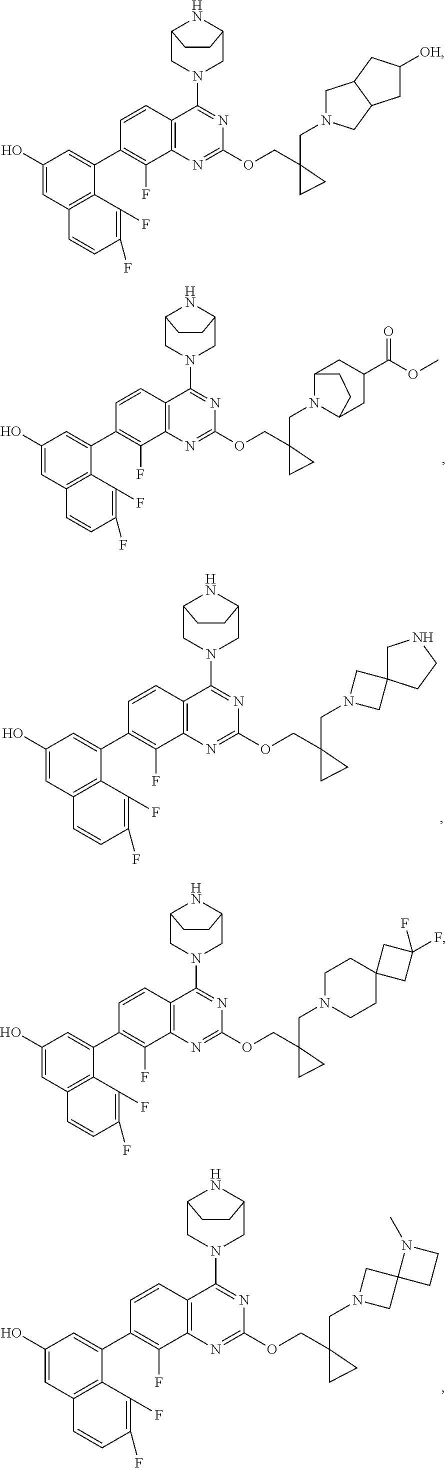 [SMILES: Oc1cc(-c2ccc3c(N4CC5CCC(C4)N5)nc(OCC4(CN5CC6CC(O)CC6C5)CC4)nc3c2F)c2c(F)c(F)ccc2c1 ; COC(=O)C1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCNC6)C5)CC4)nc3c2F)c2c(F)c(F)ccc2c1 ; Oc1cc(-c2ccc3c(N4CC5CCC(C4)N5)nc(OCC4(CN5CCC6(CC5)CC(F)(F)C6)CC4)nc3c2F)c2c(F)c(F)ccc2c1 ; CN1CCC12CN(CC1(COc3nc(N4CC5CCC(C4)N5)c4ccc(-c5cc(O)cc6ccc(F)c(F)c56)c(F)c4n3)CC1)C2]

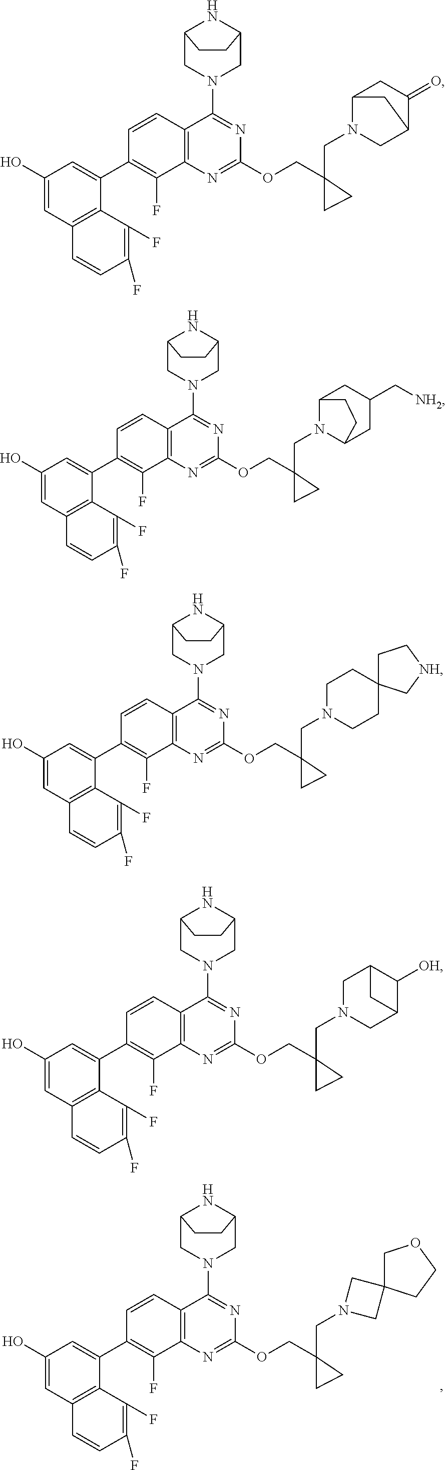 [SMILES: O=C1CC2CC1CN2CC1(COc2nc(N3CC4CCC(C3)N4)c3ccc(-c4cc(O)cc5ccc(F)c(F)c45)c(F)c3n2)CC1 ; NCC1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CCC6(CCNC6)CC5)CC4)nc3c2F)c2c(F)c(F)ccc2c1 ; Oc1cc(-c2ccc3c(N4CC5CCC(C4)N5)nc(OCC4(CN5CC6CC(C5)C6O)CC4)nc3c2F)c2c(F)c(F)ccc2c1 ; Oc1cc(-c2ccc3c(N4CC5CCC(C4)N5)nc(OCC4(CN5CC6(CCOC6)C5)CC4)nc3c2F)c2c(F)c(F)ccc2c1]

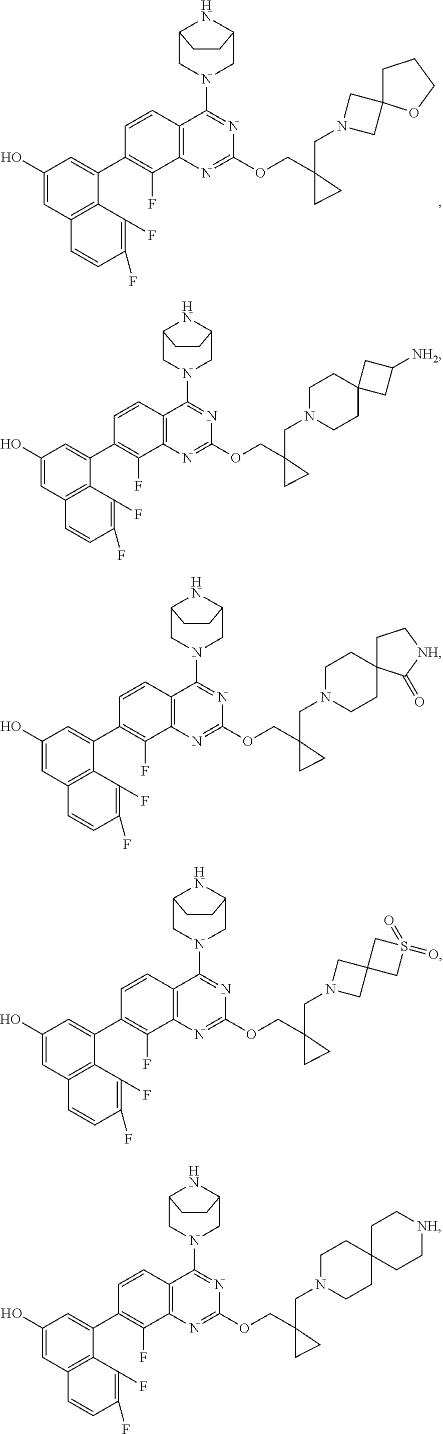 [SMILES: Oc1cc(-c2ccc3c(N4CC5CCC(C4)N5)nc(OCC4(CN5CC6(CCCO6)C5)CC4)nc3c2F)c2c(F)c(F)ccc2c1 ; NC1CC2(CCN(CC3(COc4nc(N5CC6CCC(C5)N6)c5ccc(-c6cc(O)cc7ccc(F)c(F)c67)c(F)c5n4)CC3)CC2)C1 ; O=C1NCCC12CCN(CC1(COc3nc(N4CC5CCC(C4)N5)c4ccc(-c5cc(O)cc6ccc(F)c(F)c56)c(F)c4n3)CC1)CC2 ; O=S1(=O)CC2(CN(CC3(COc4nc(N5CC6CCC(C5)N6)c5ccc(-c6cc(O)cc7ccc(F)c(F)c67)c(F)c5n4)CC3)C2)C1 ; Oc1cc(-c2ccc3c(N4CC5CCC(C4)N5)nc(OCC4(CN5CCC6(CCNCC6)CC5)CC4)nc3c2F)c2c(F)c(F)ccc2c1]

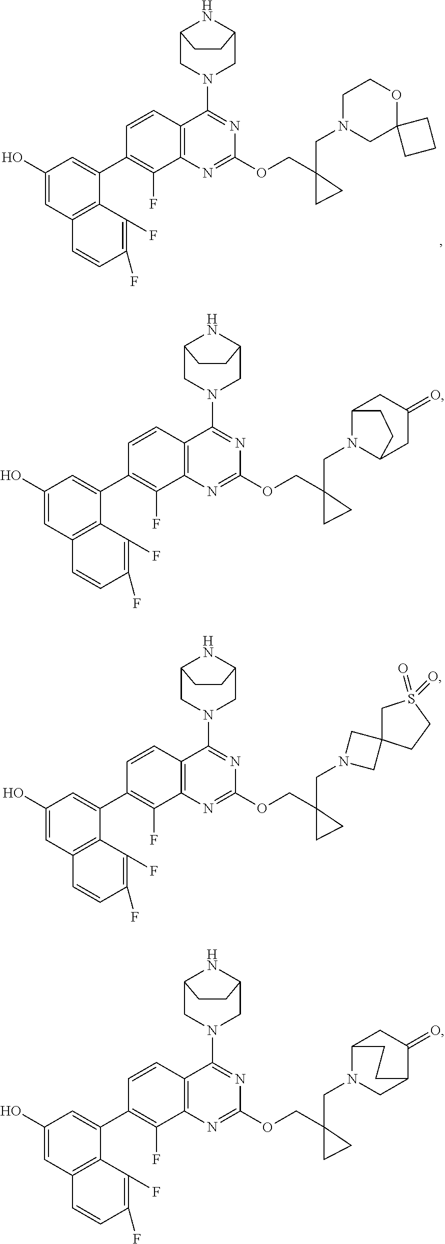 [SMILES: Oc1cc(-c2ccc3c(N4CC5CCC(C4)N5)nc(OCC4(CN5CCOC6(CCC6)C5)CC4)nc3c2F)c2c(F)c(F)ccc2c1 ; O=C1CC2CCC(C1)N2CC1(COc2nc(N3CC4CCC(C3)N4)c3ccc(-c4cc(O)cc5ccc(F)c(F)c45)c(F)c3n2)CC1 ; O=S1(=O)CCC2(CN(CC3(COc4nc(N5CC6CCC(C5)N6)c5ccc(-c6cc(O)cc7ccc(F)c(F)c67)c(F)c5n4)CC3)C2)C1 ; O=C1CC2CCC1CN2CC1(COc2nc(N3CC4CCC(C3)N4)c3ccc(-c4cc(O)cc5ccc(F)c(F)c45)c(F)c3n2)CC1]

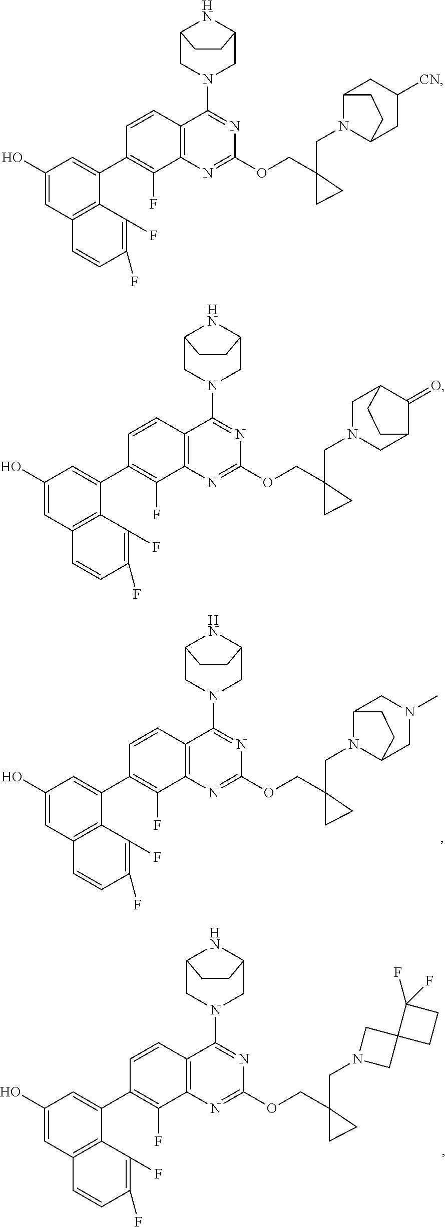 [SMILES: N#CC1CC2CCC(C1)N2CC1(COc2nc(N3CC4CCC(C3)N4)c3ccc(-c4cc(O)cc5ccc(F)c(F)c45)c(F)c3n2)CC1 ; O=C1C2CCC1CN(CC1(COc3nc(N4CC5CCC(C4)N5)c4ccc(-c5cc(O)cc6ccc(F)c(F)c56)c(F)c4n3)CC1)C2 ; CN1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCC6(F)F)C5)CC4)nc3c2F)c2c(F)c(F)ccc2c1]

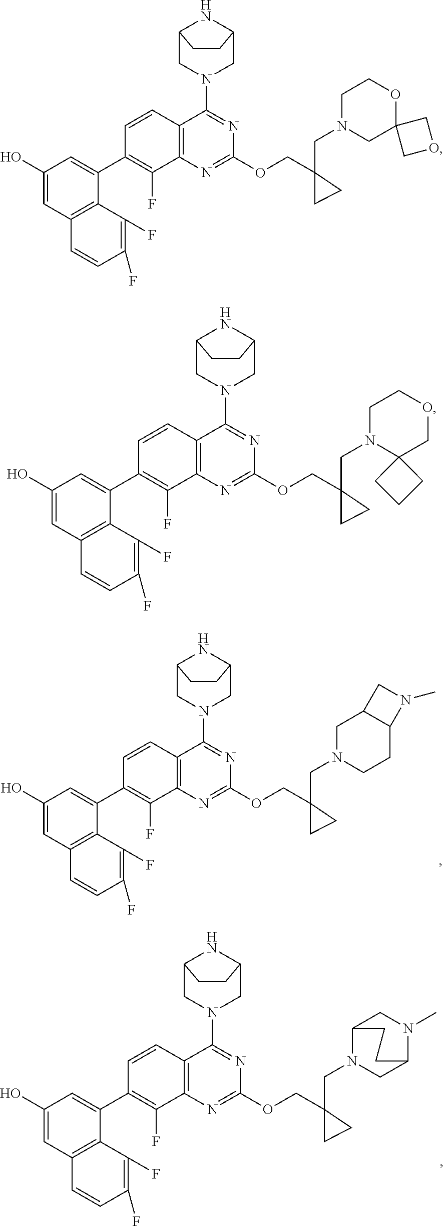 [SMILES: Oc1cc(-c2ccc3c(N4CC5CCC(C4)N5)nc(OCC4(CN5CCOC6(COC6)C5)CC4)nc3c2F)c2c(F)c(F)ccc2c1 ; Oc1cc(-c2ccc3c(N4CC5CCC(C4)N5)nc(OCC4(CN5CCOCC56CCC6)CC4)nc3c2F)c2c(F)c(F)ccc2c1 ; CN1CC2CN(CC3(COc4nc(N5CC6CCC(C5)N6)c5ccc(-c6cc(O)cc7ccc(F)c(F)c67)c(F)c5n4)CC3)CCC21 ; CN1CC2CCC1CN2CC1(COc2nc(N3CC4CCC(C3)N4)c3ccc(-c4cc(O)cc5ccc(F)c(F)c45)c(F)c3n2)CC1]

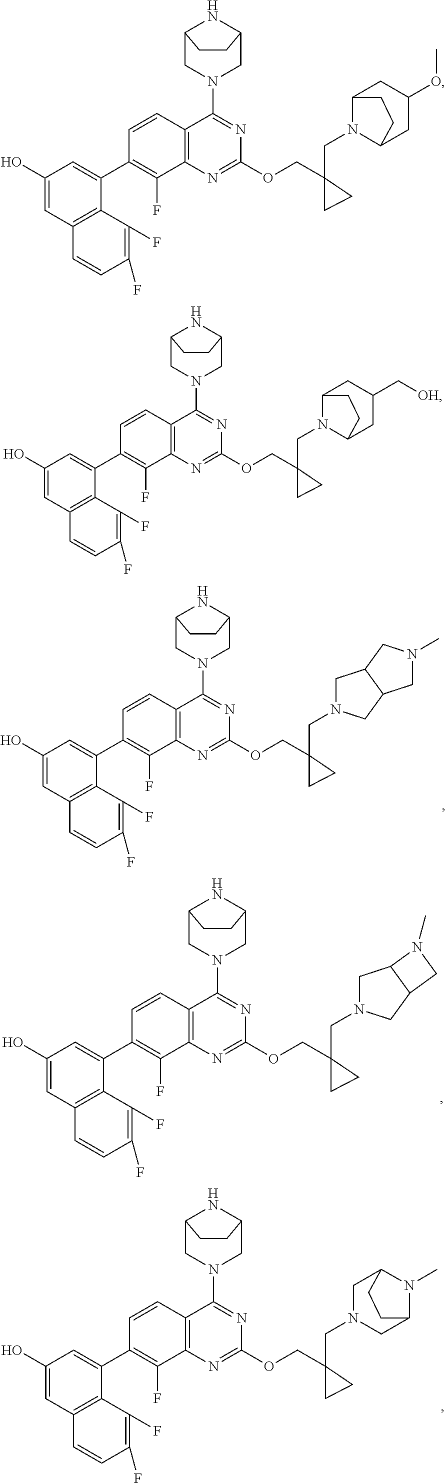 [SMILES: COC1CC2CCC(C1)N2CC1(COc2nc(N3CC4CCC(C3)N4)c3ccc(-c4cc(O)cc5ccc(F)c(F)c45)c(F)c3n2)CC1 ; OCC1CC2CCC(C1)N2CC1(COc2nc(N3CC4CCC(C3)N4)c3ccc(-c4cc(O)cc5ccc(F)c(F)c45)c(F)c3n2)CC1 ; CN1CC2CN(CC3(COc4nc(N5CC6CCC(C5)N6)c5ccc(-c6cc(O)cc7ccc(F)c(F)c67)c(F)c5n4)CC3)CC2C1 ; CN1CC2CN(CC3(COc4nc(N5CC6CCC(C5)N6)c5ccc(-c6cc(O)cc7ccc(F)c(F)c67)c(F)c5n4)CC3)CC21 ; CN1C2CCC1CN(CC1(COc3nc(N4CC5CCC(C4)N5)c4ccc(-c5cc(O)cc6ccc(F)c(F)c56)c(F)c4n3)CC1)C2]

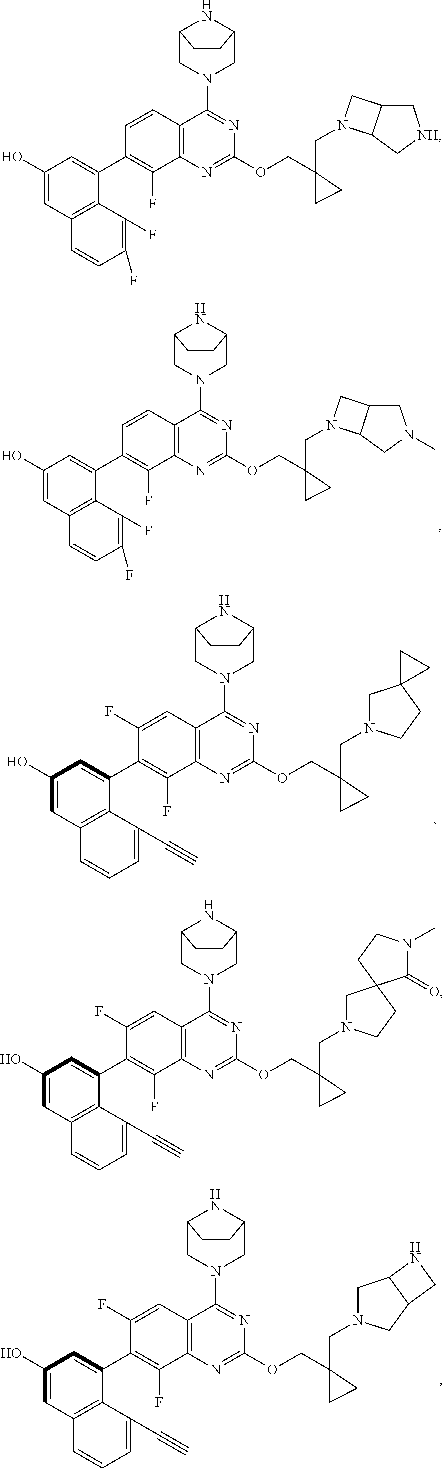

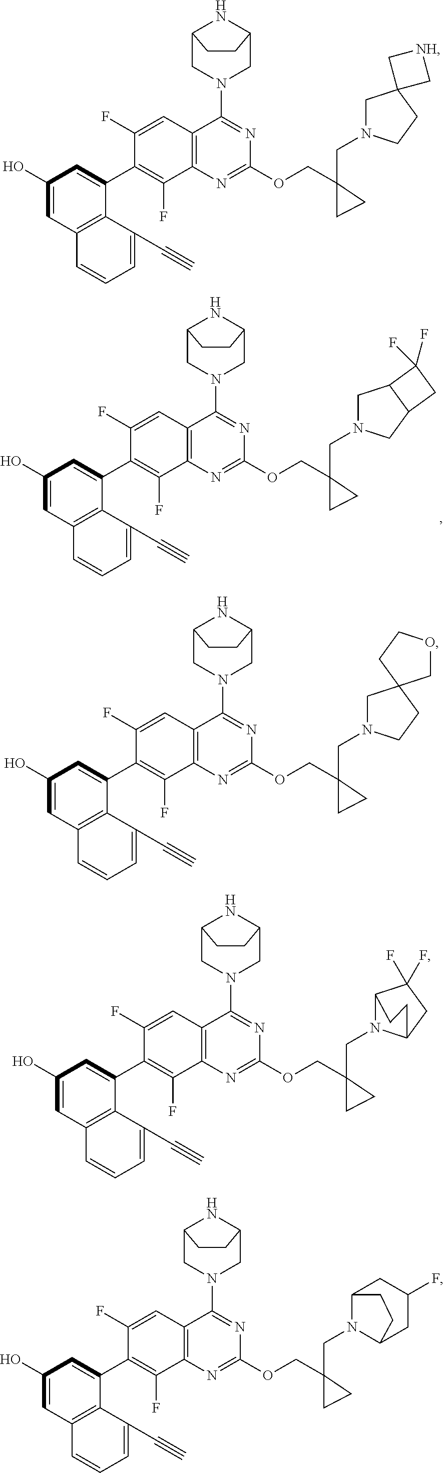

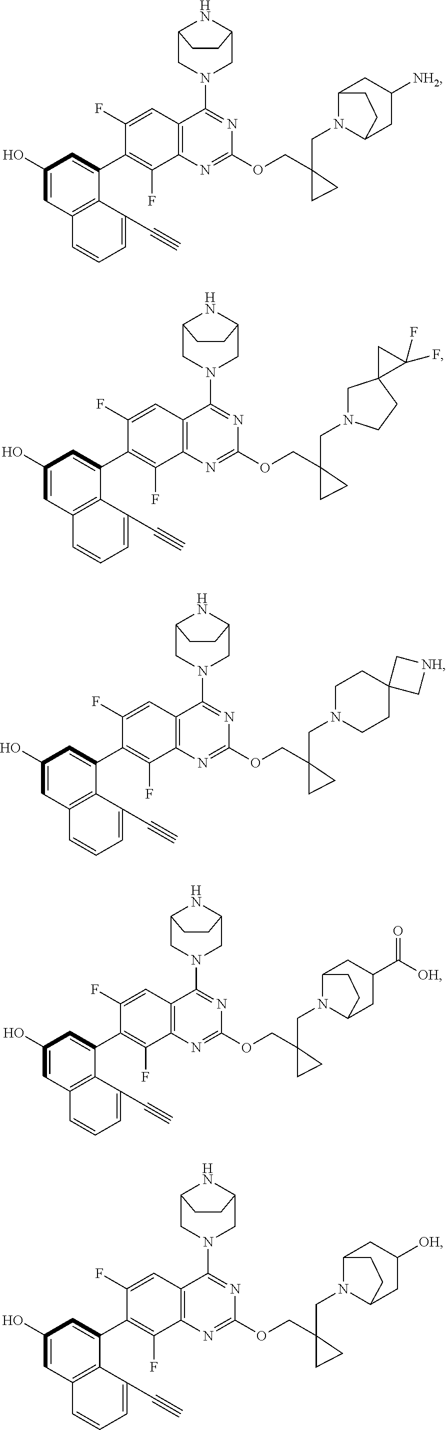

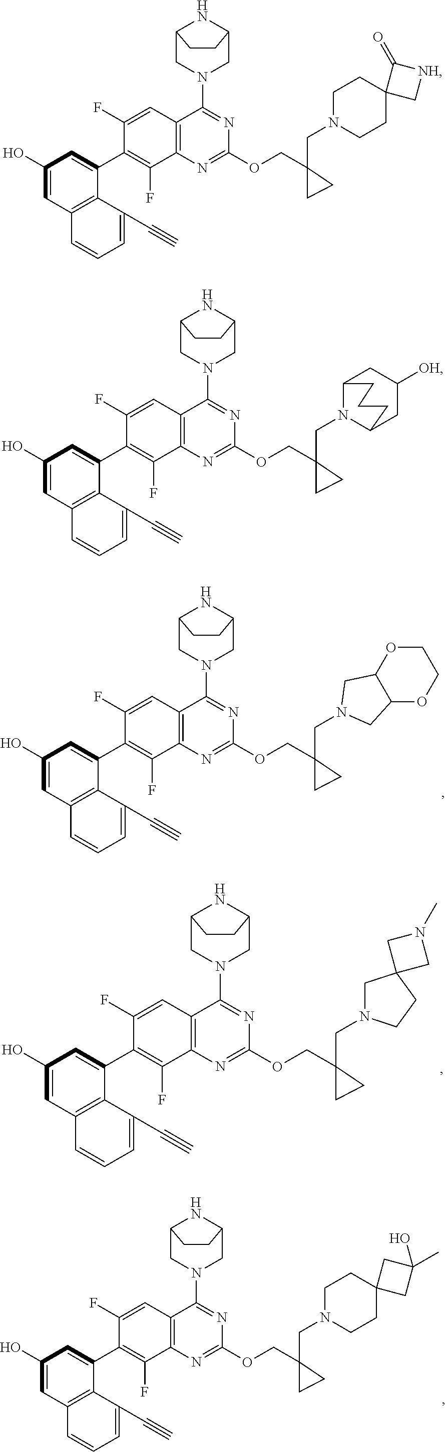

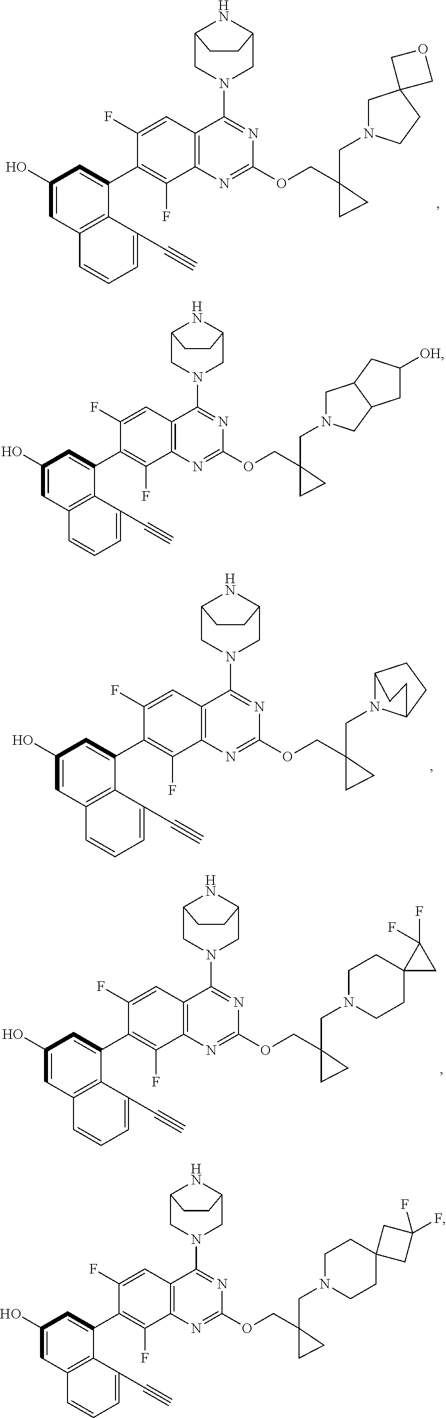

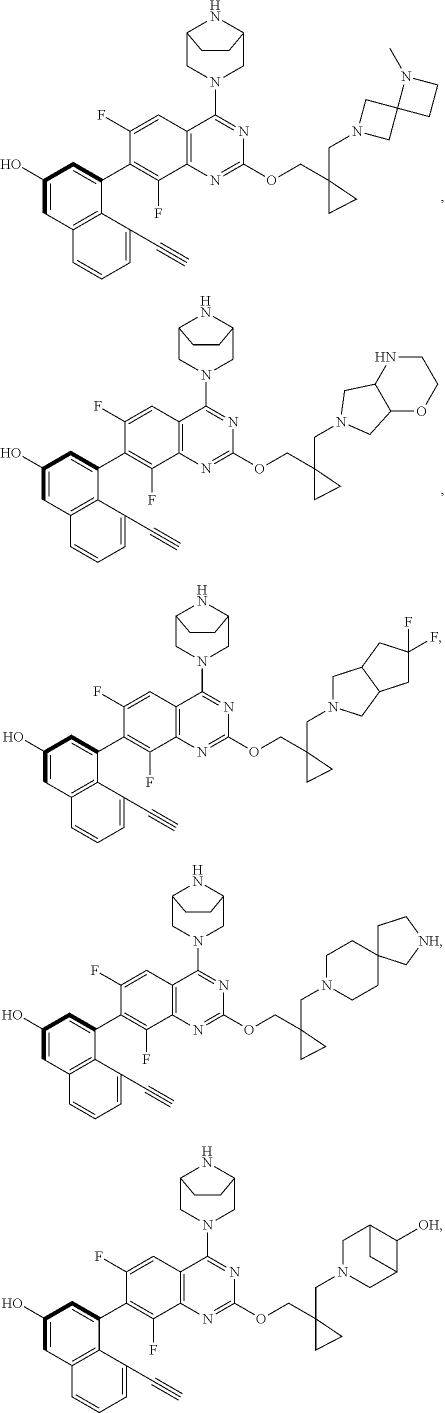

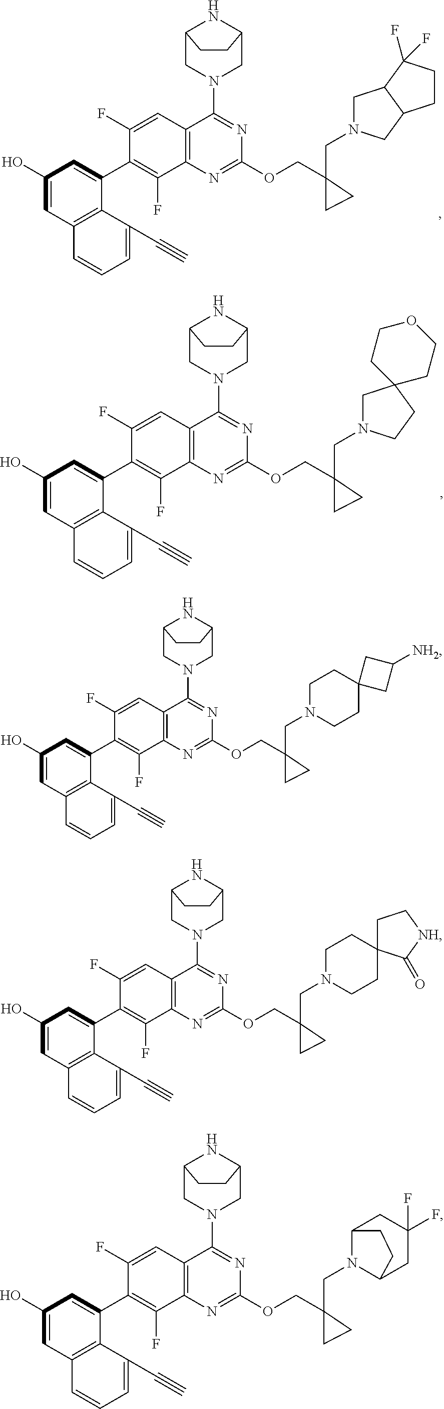

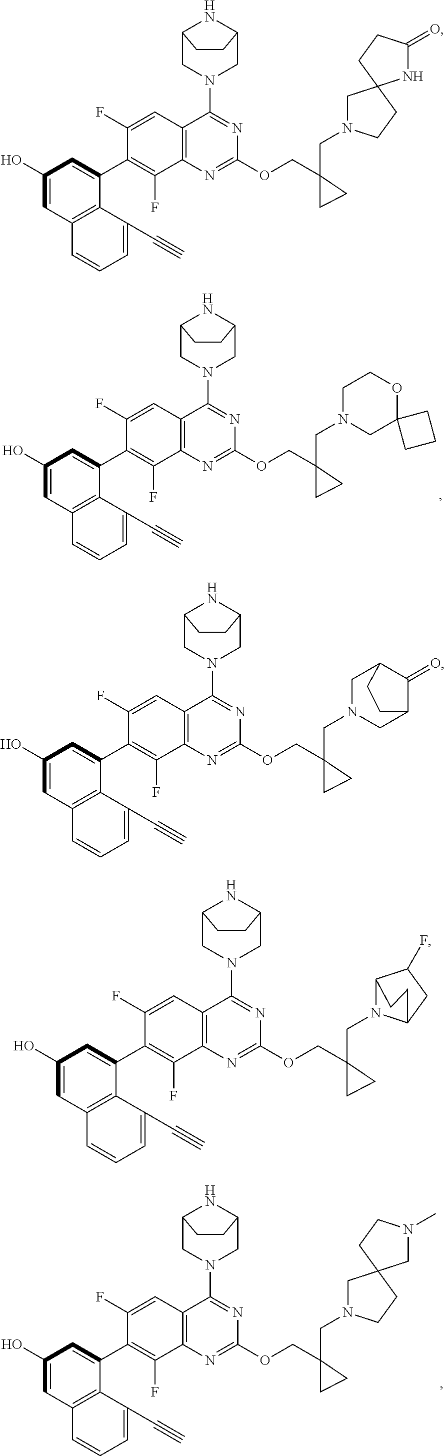

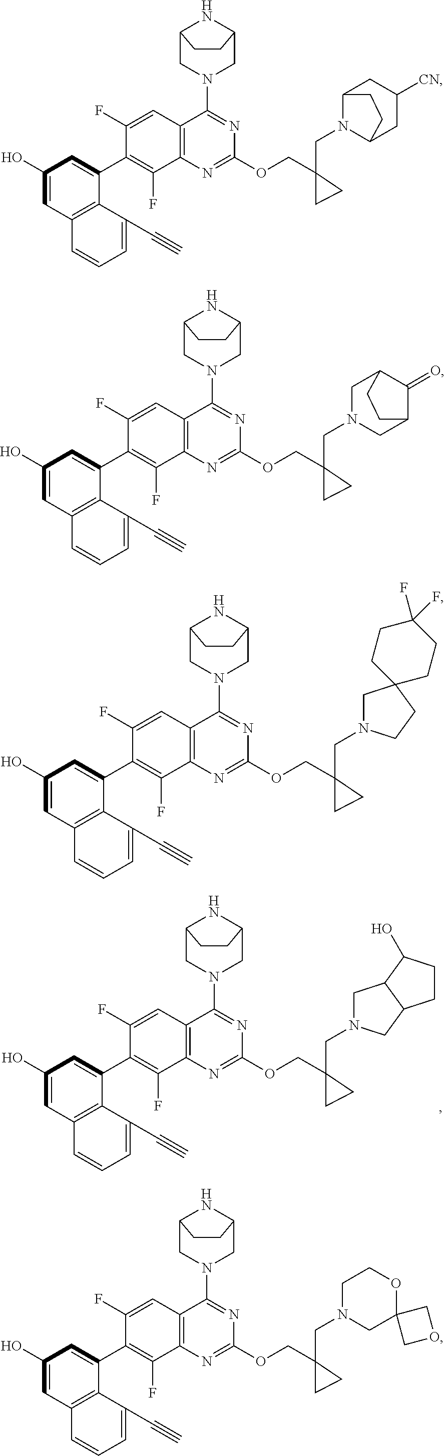

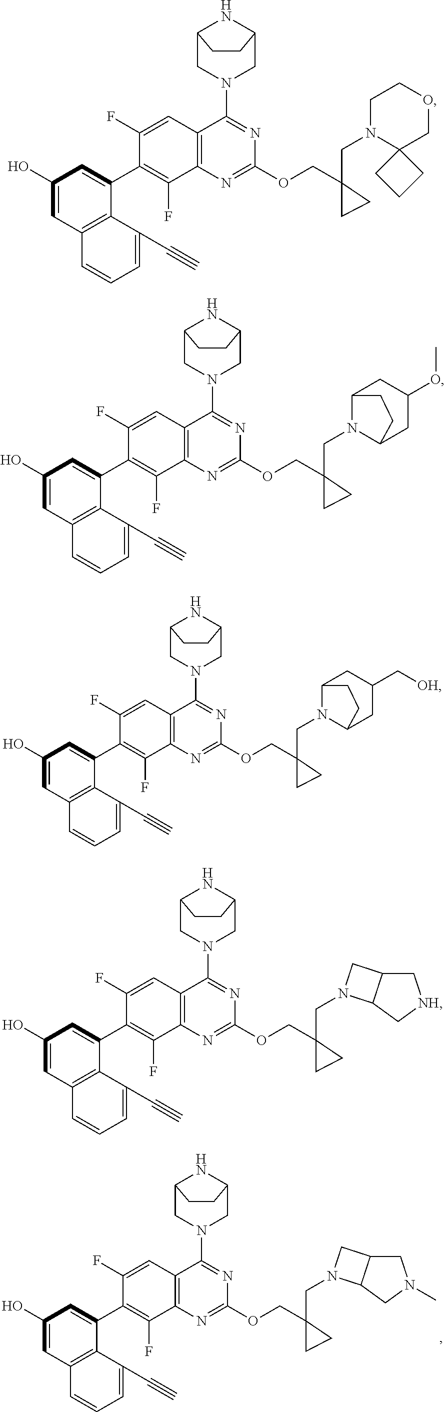

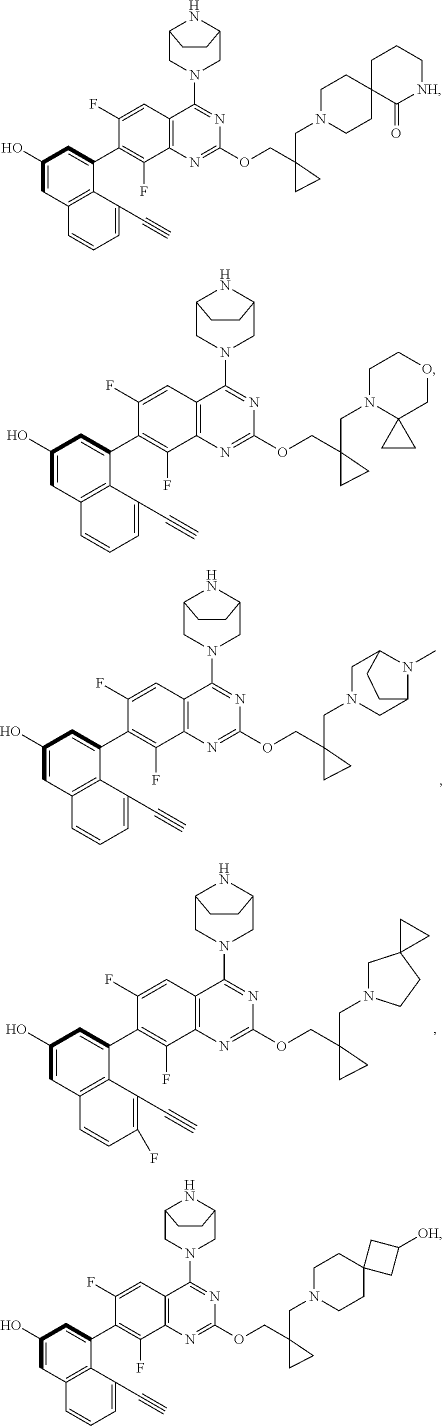

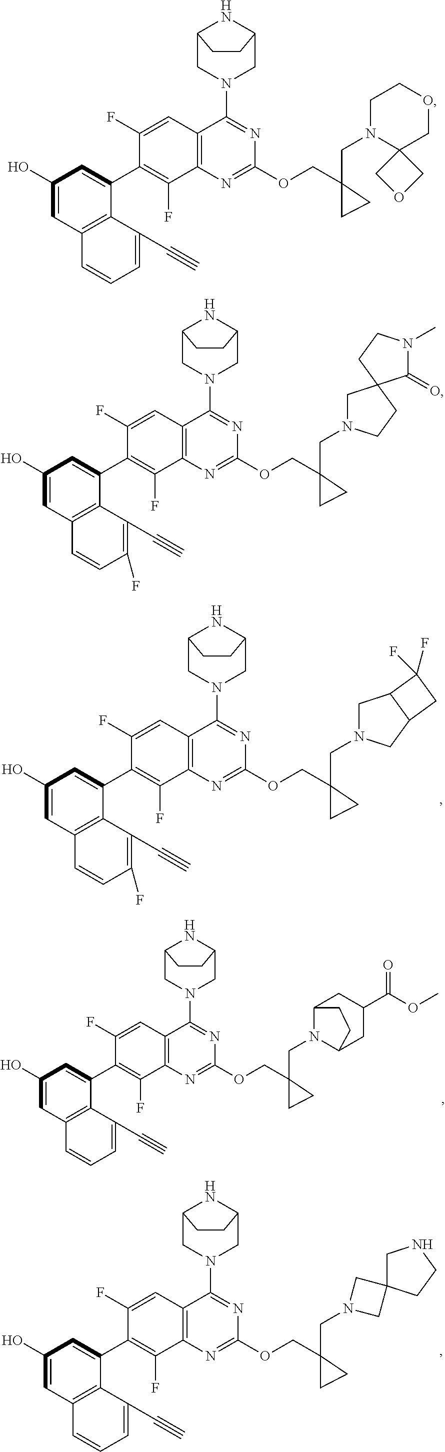

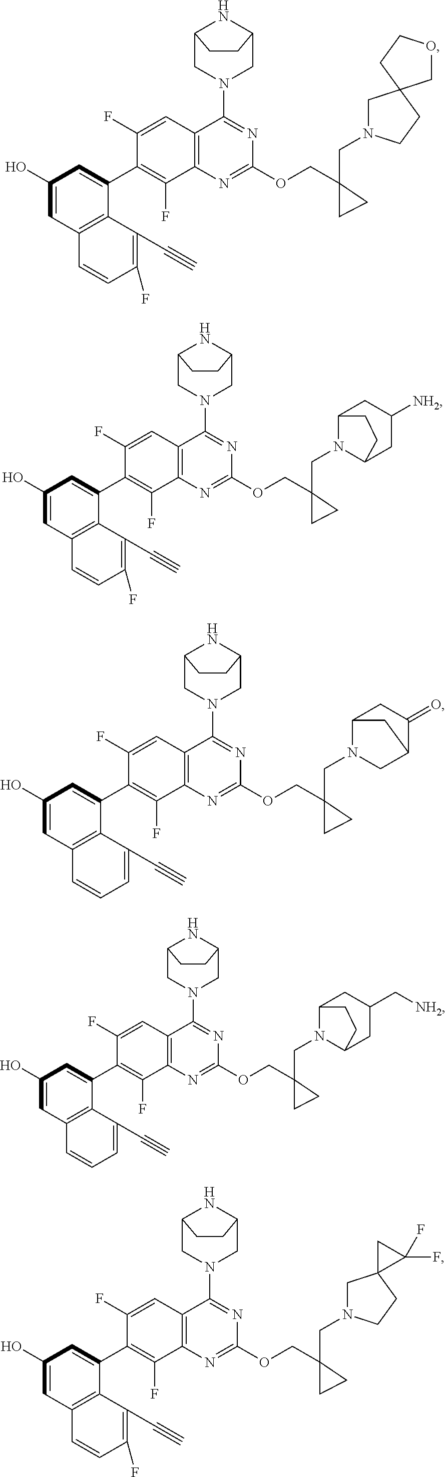

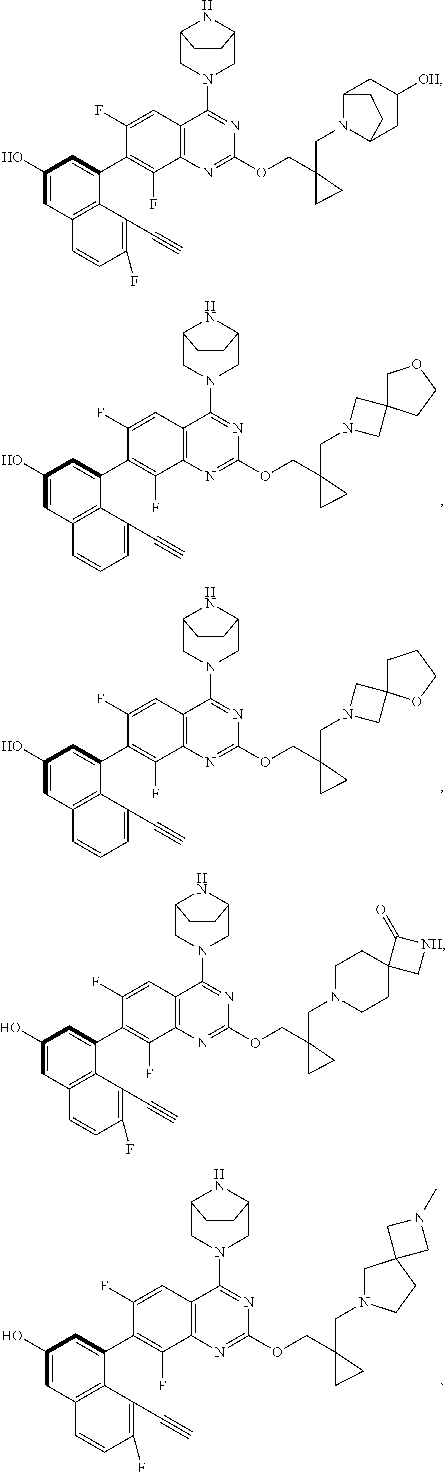

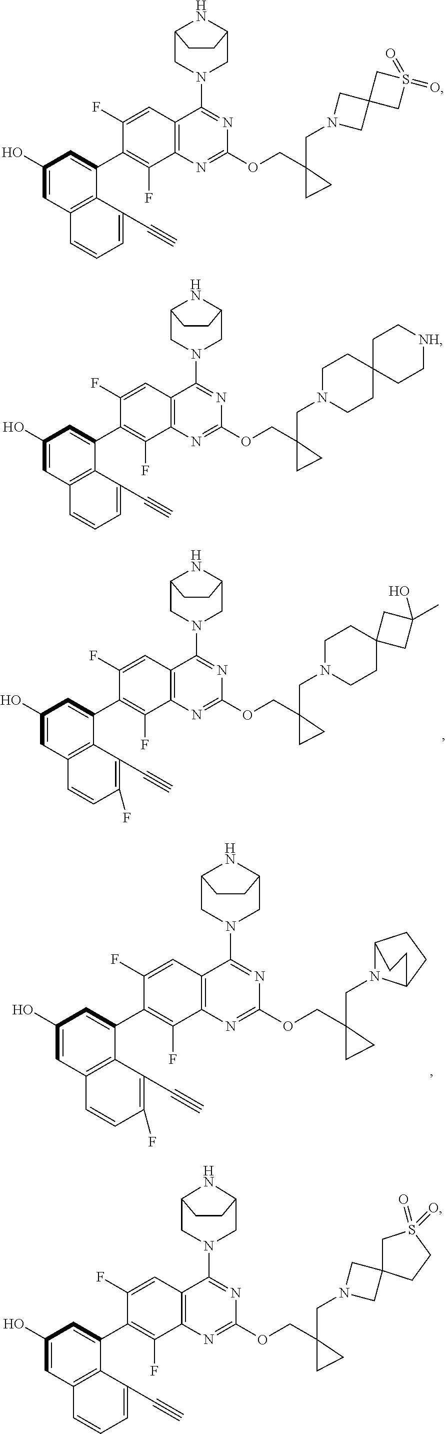

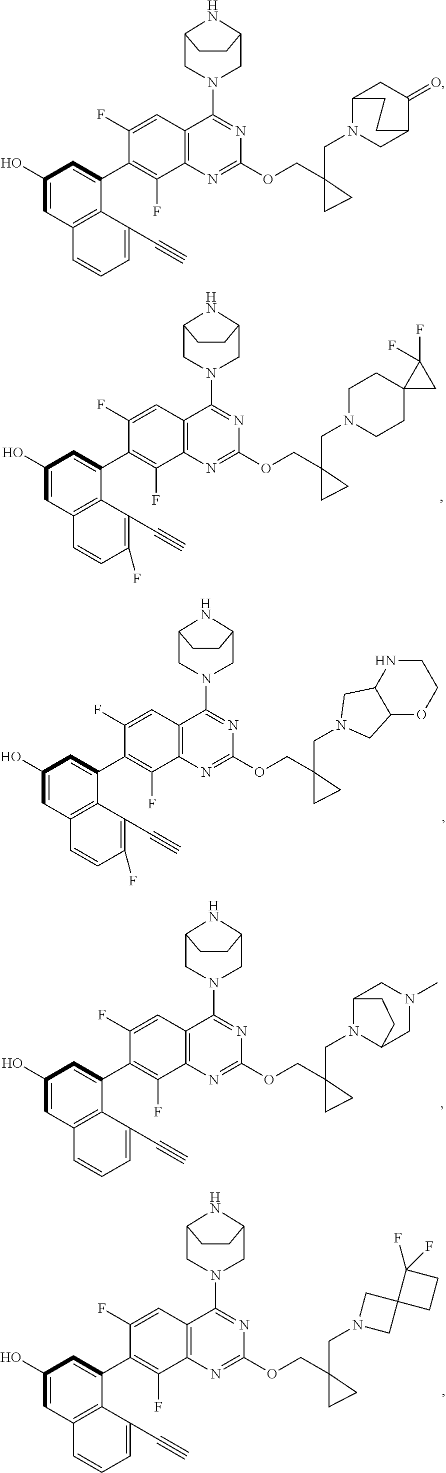

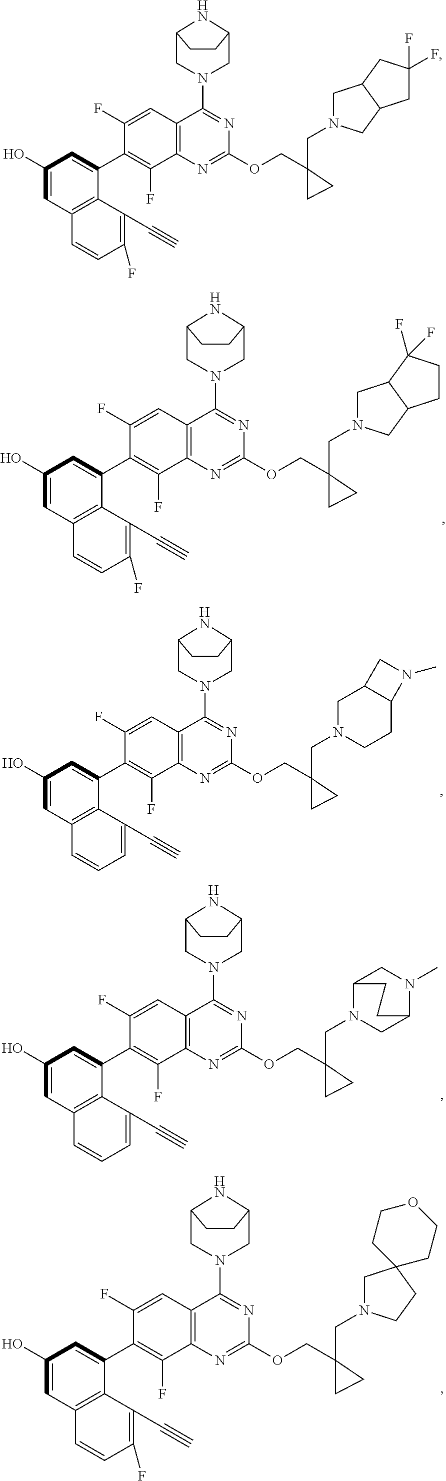

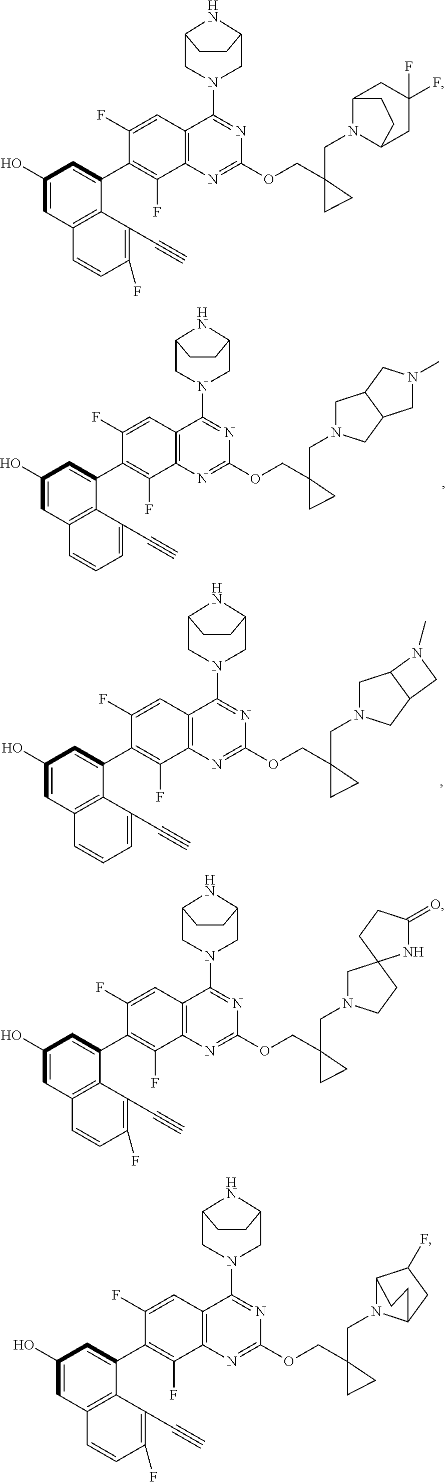

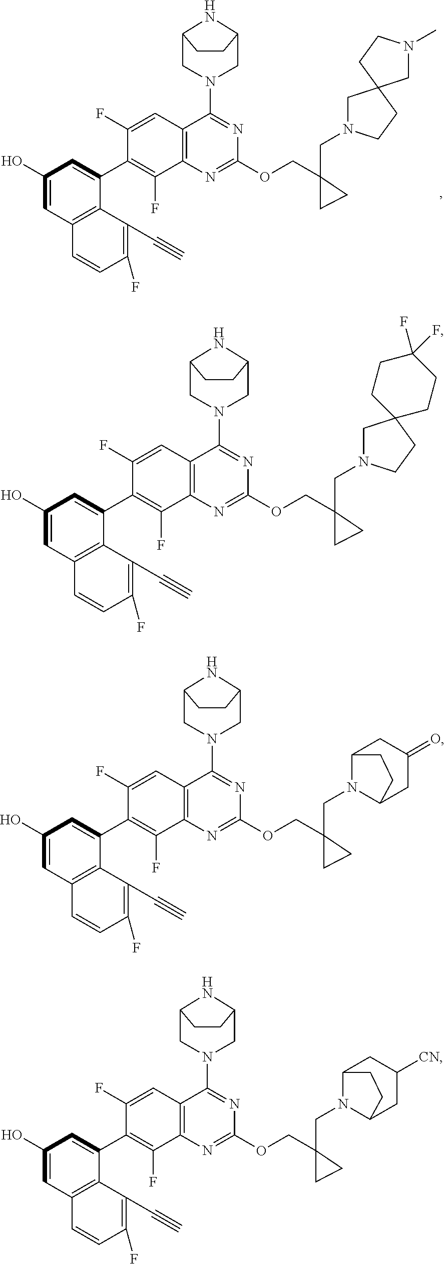

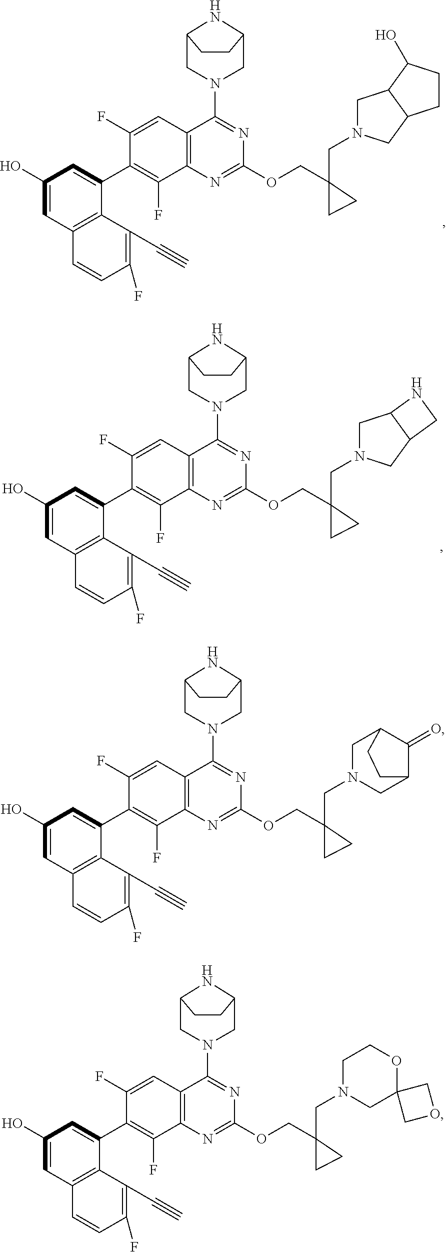

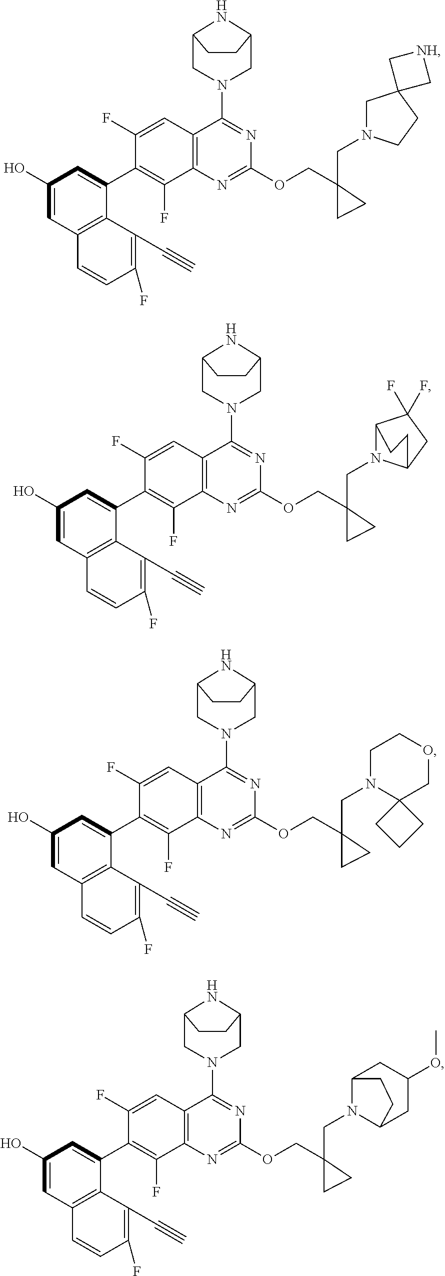

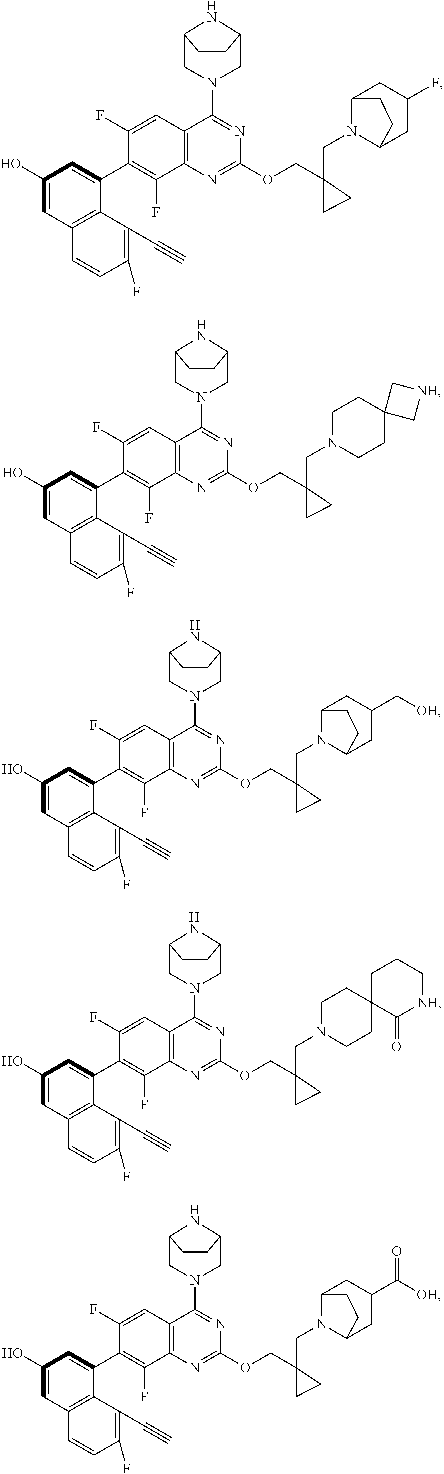

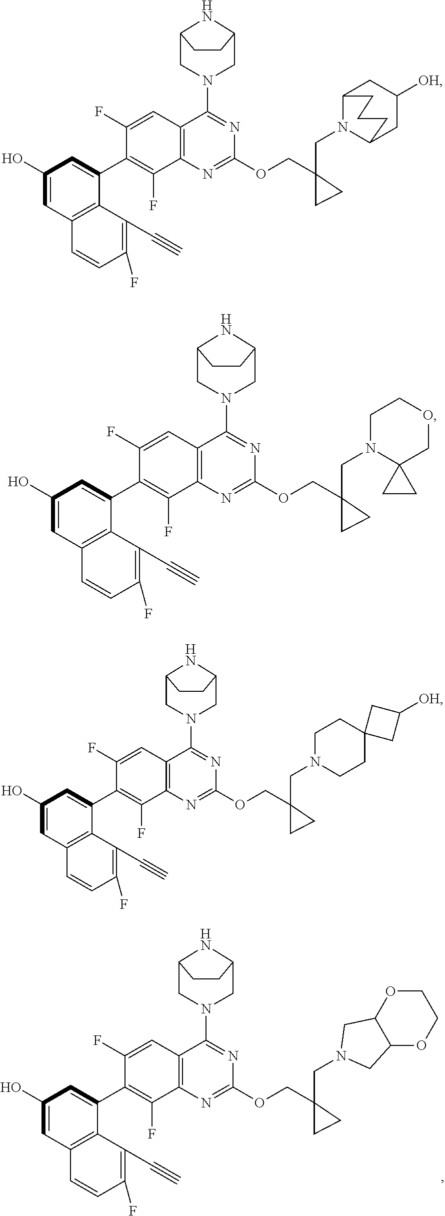

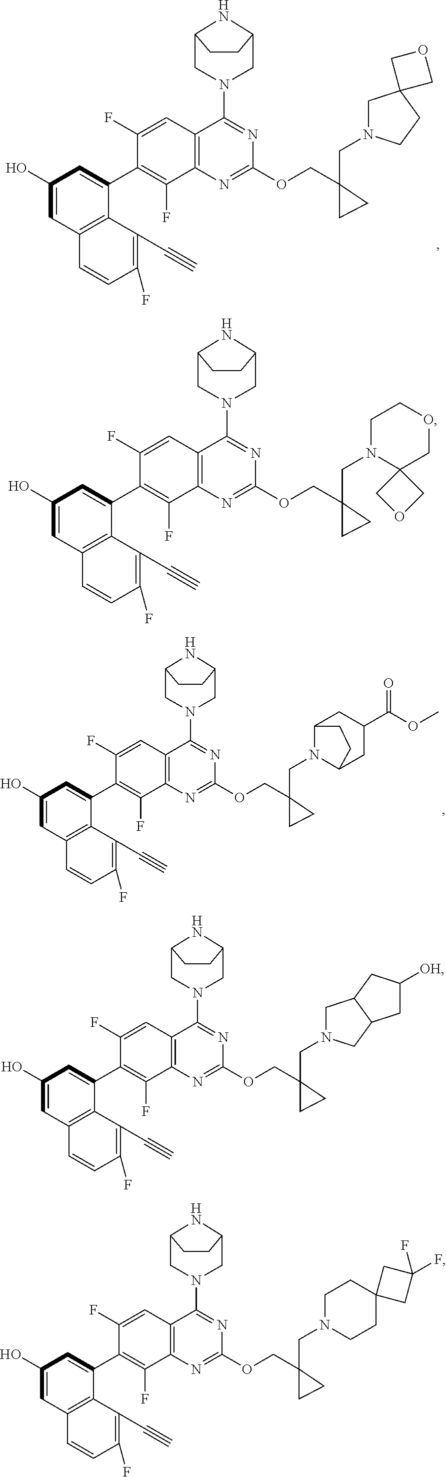

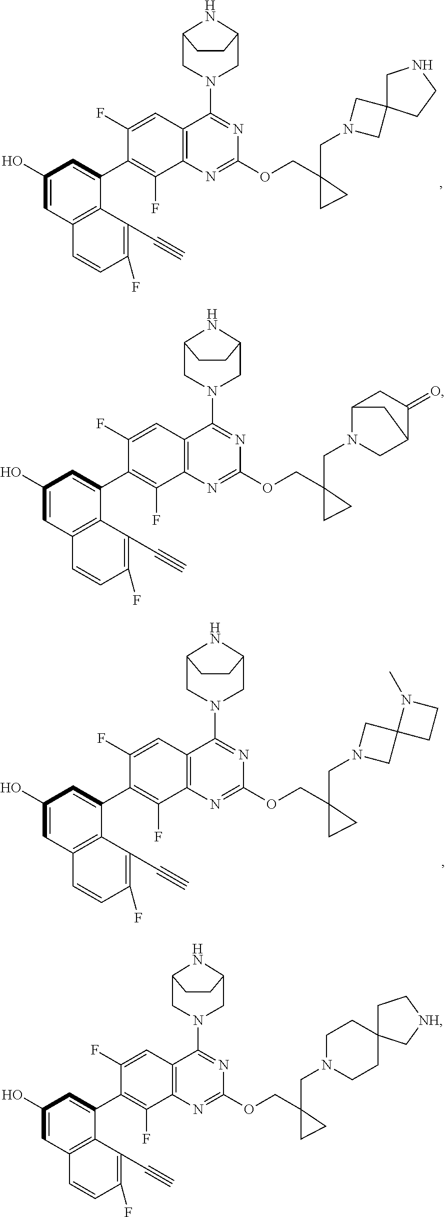

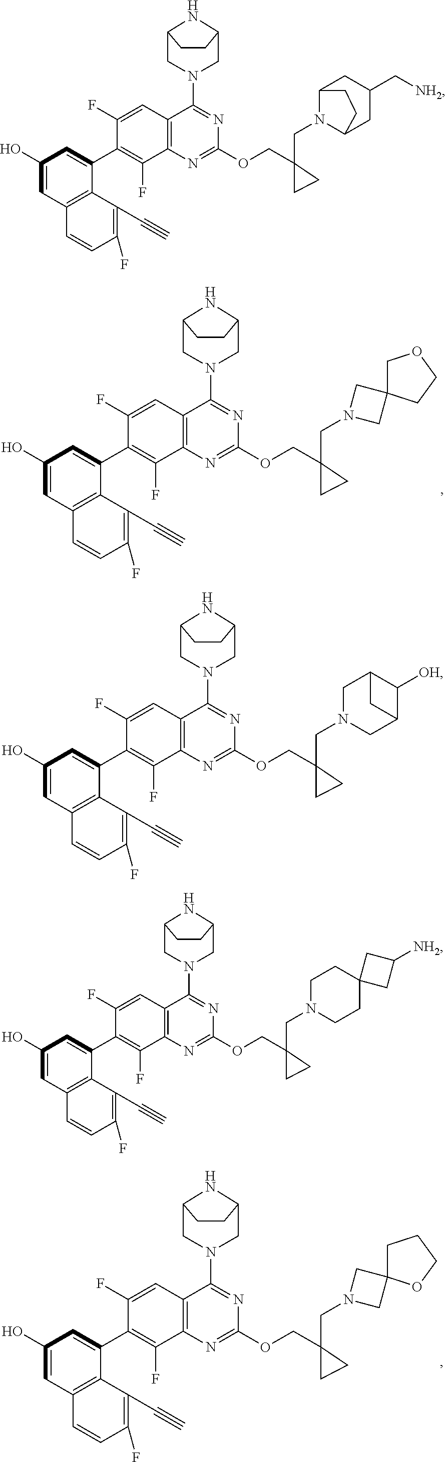

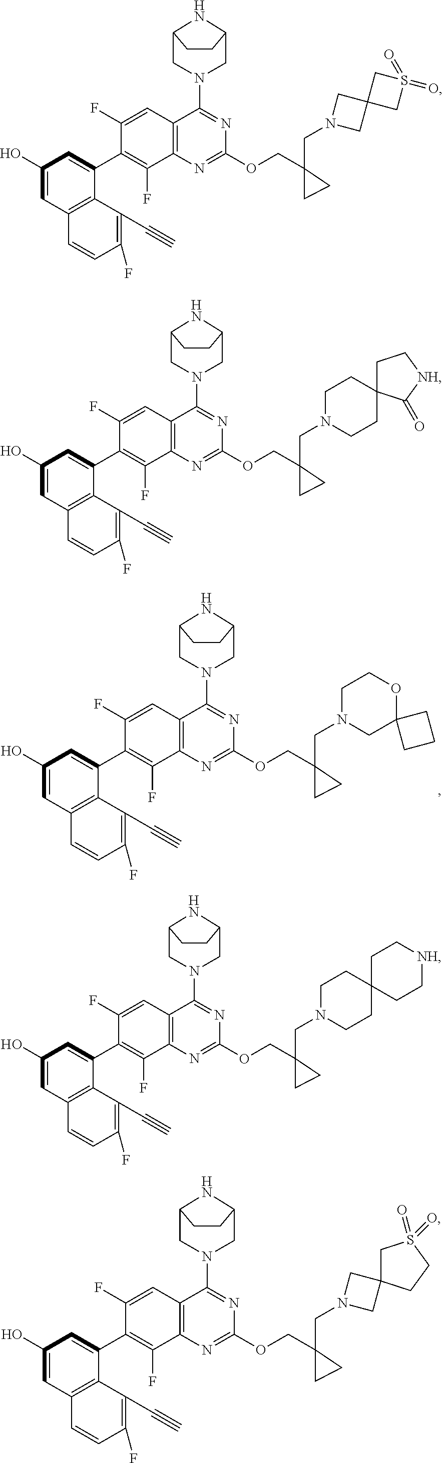

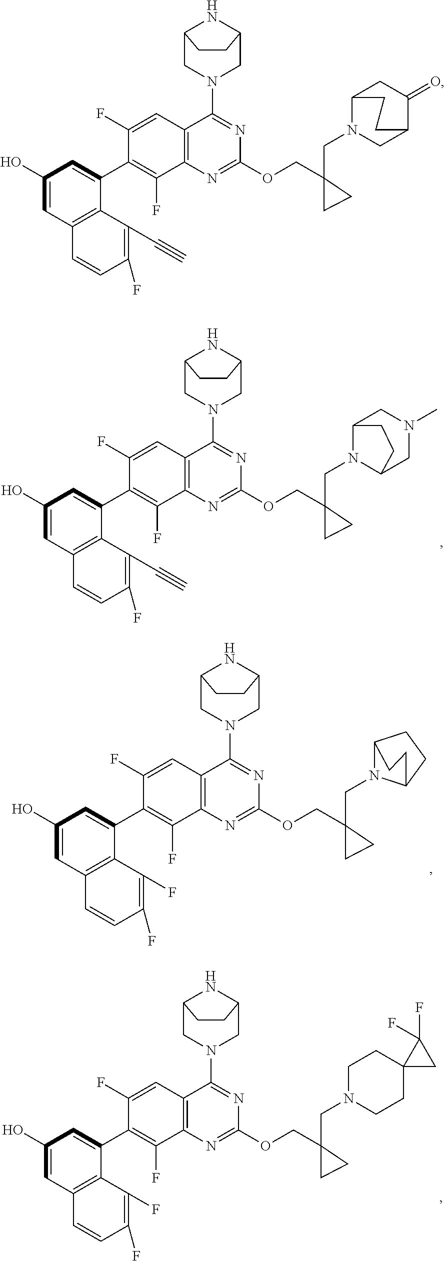

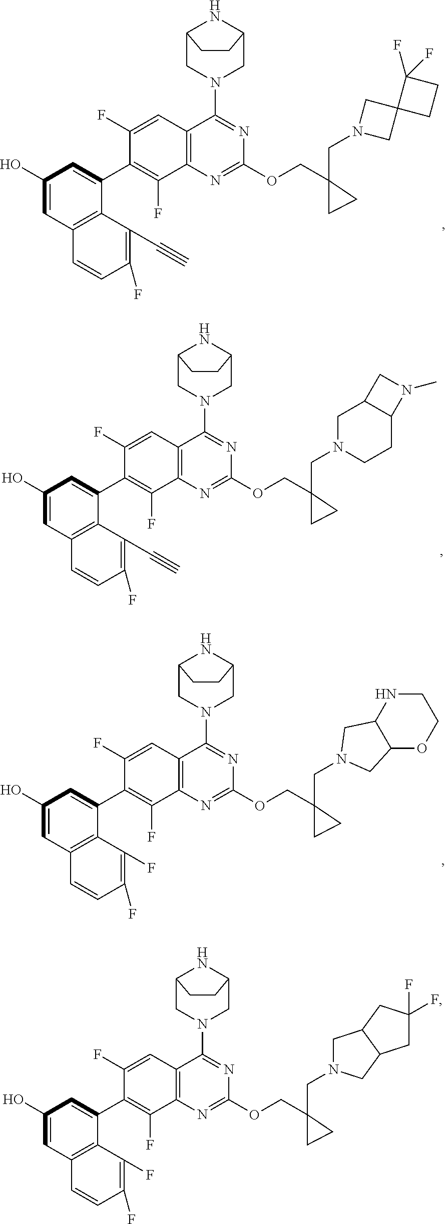

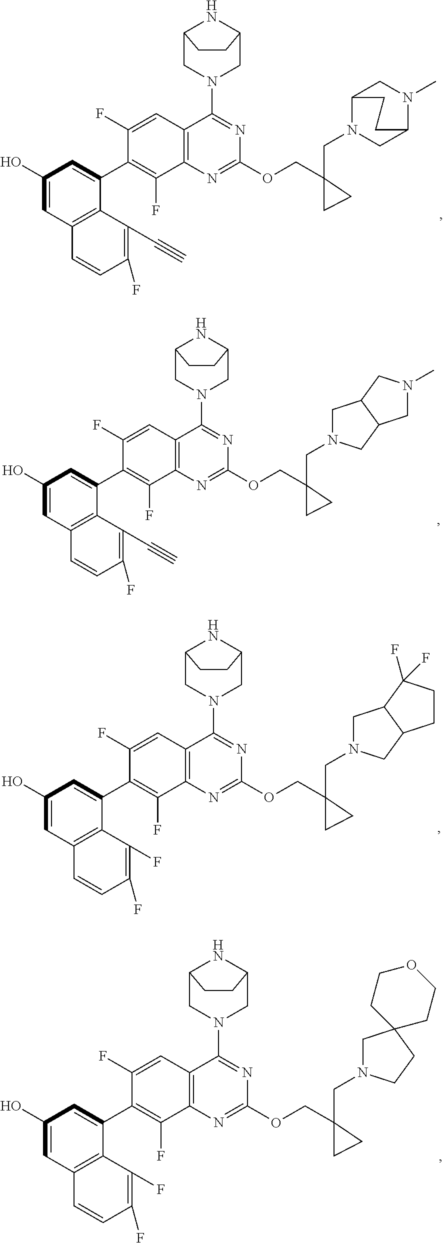

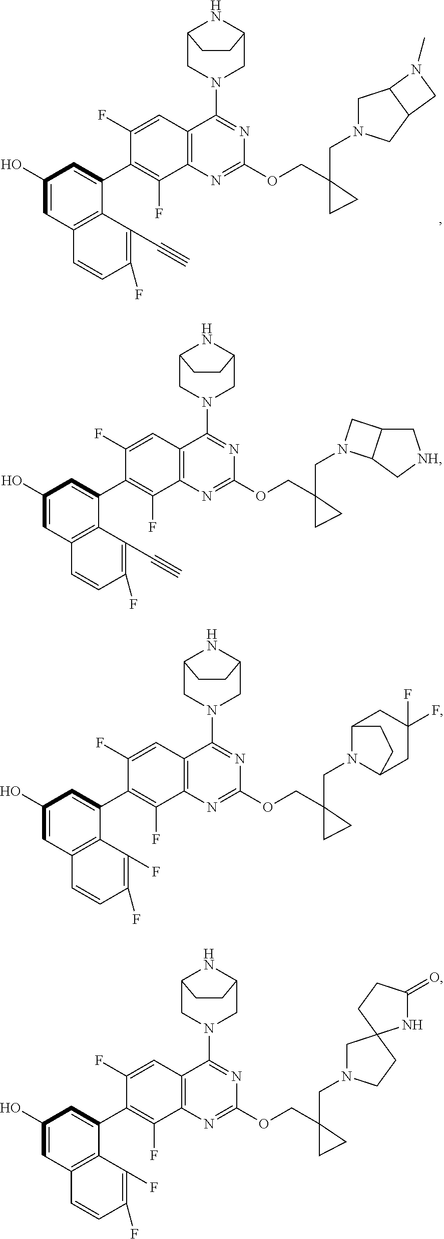

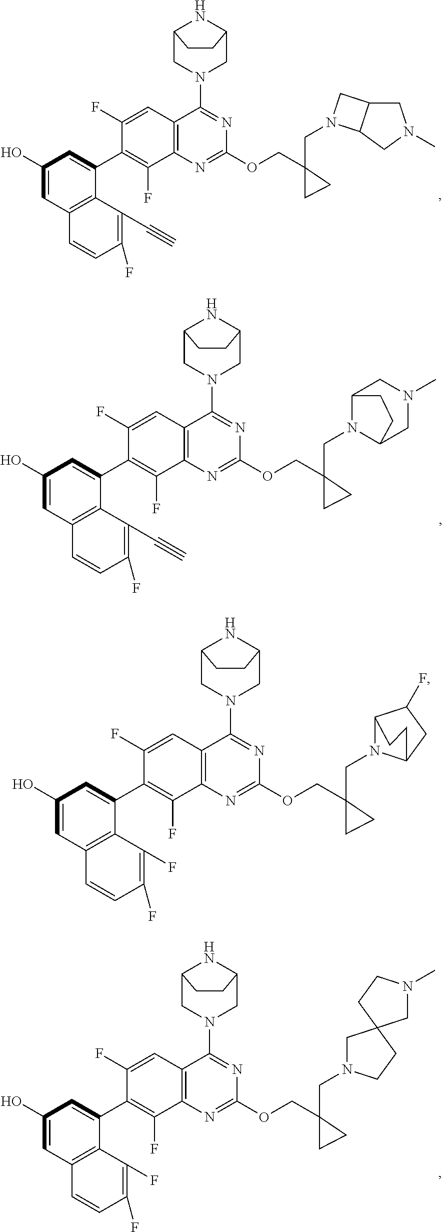

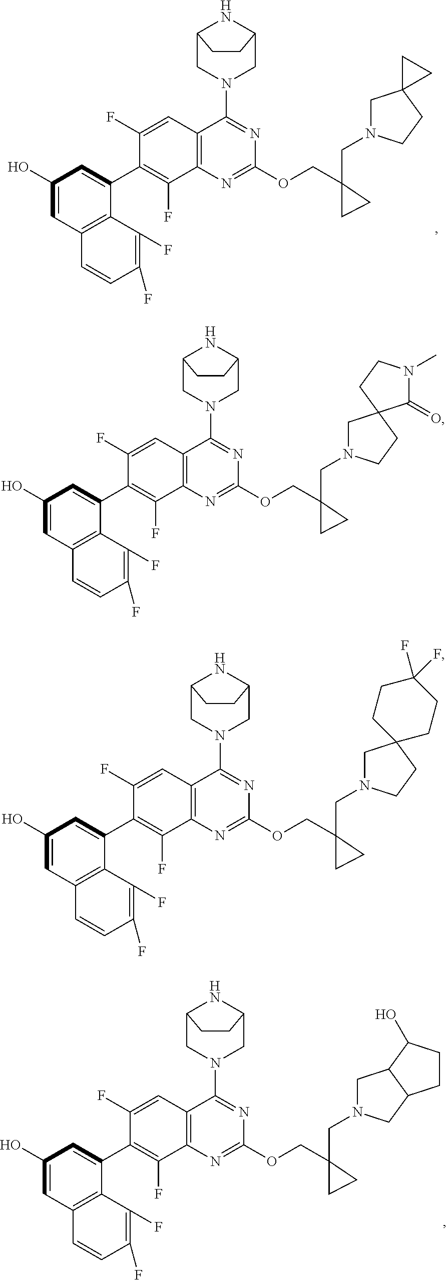

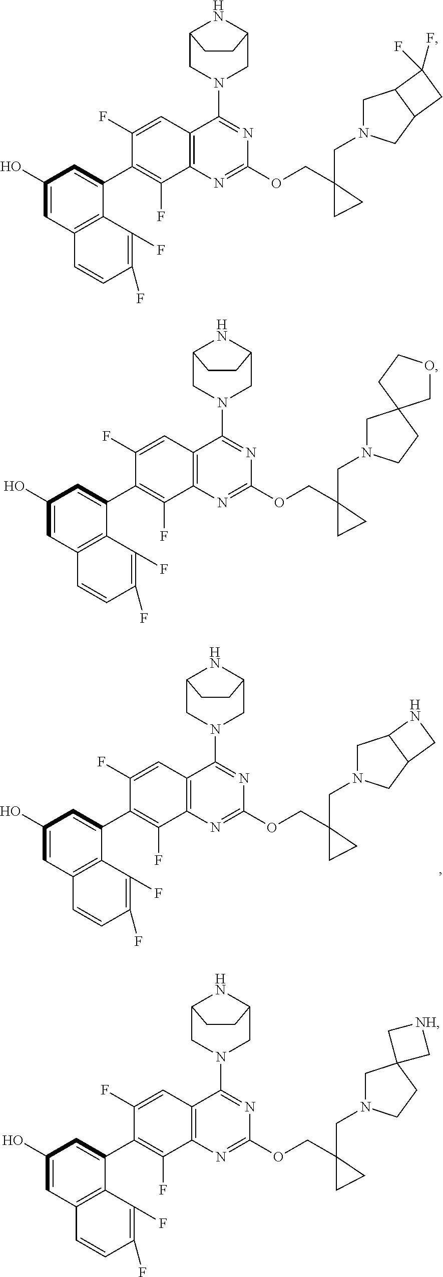

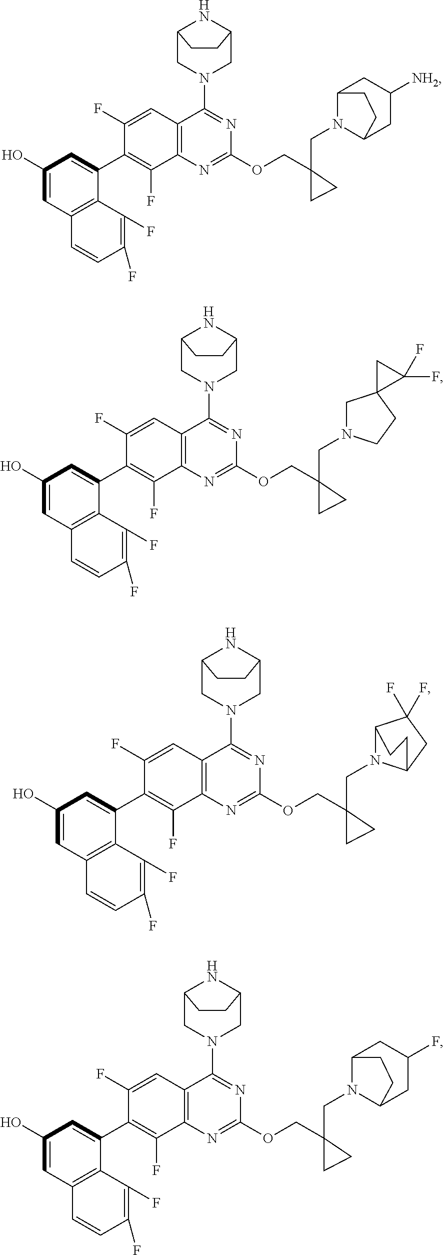

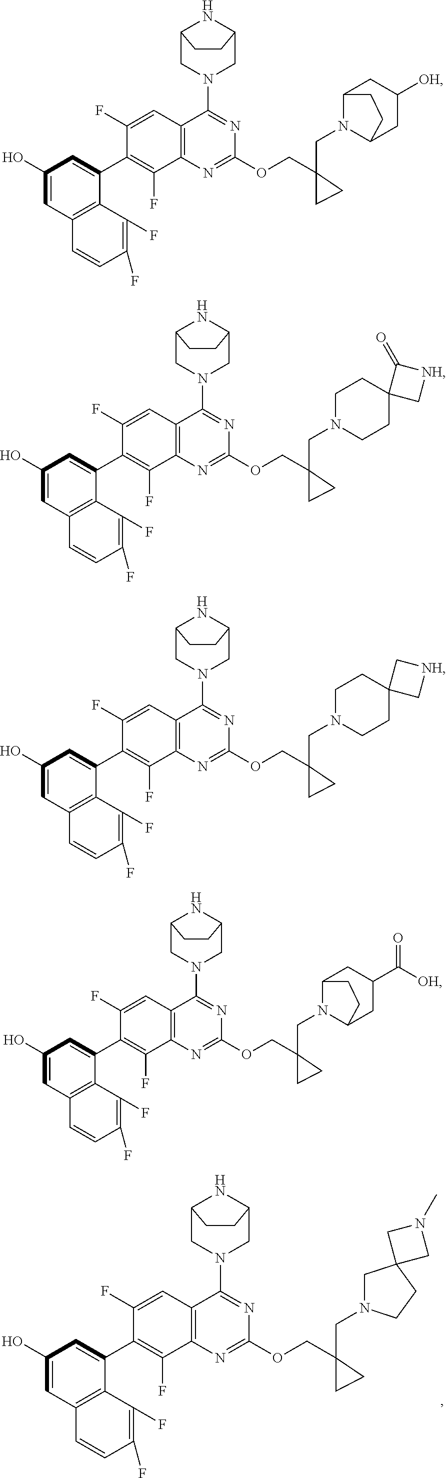

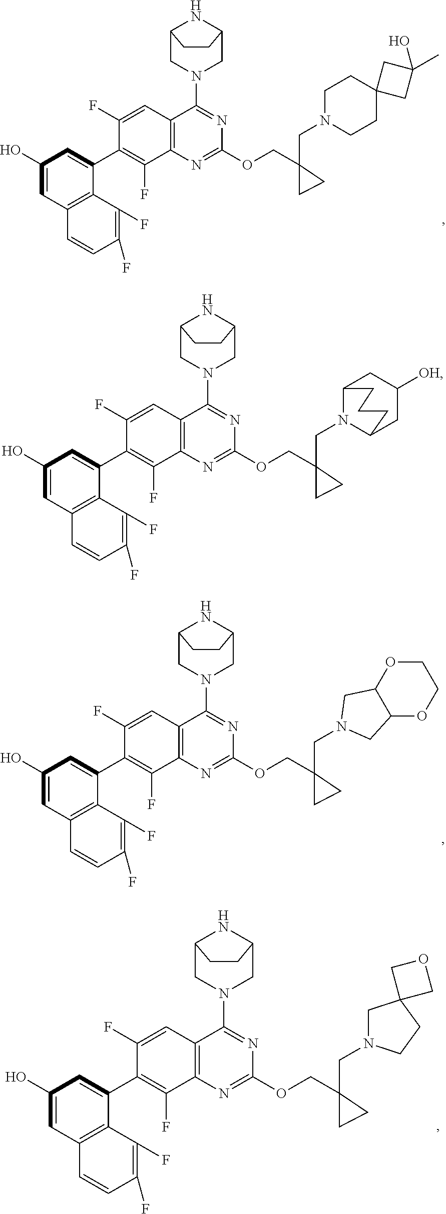

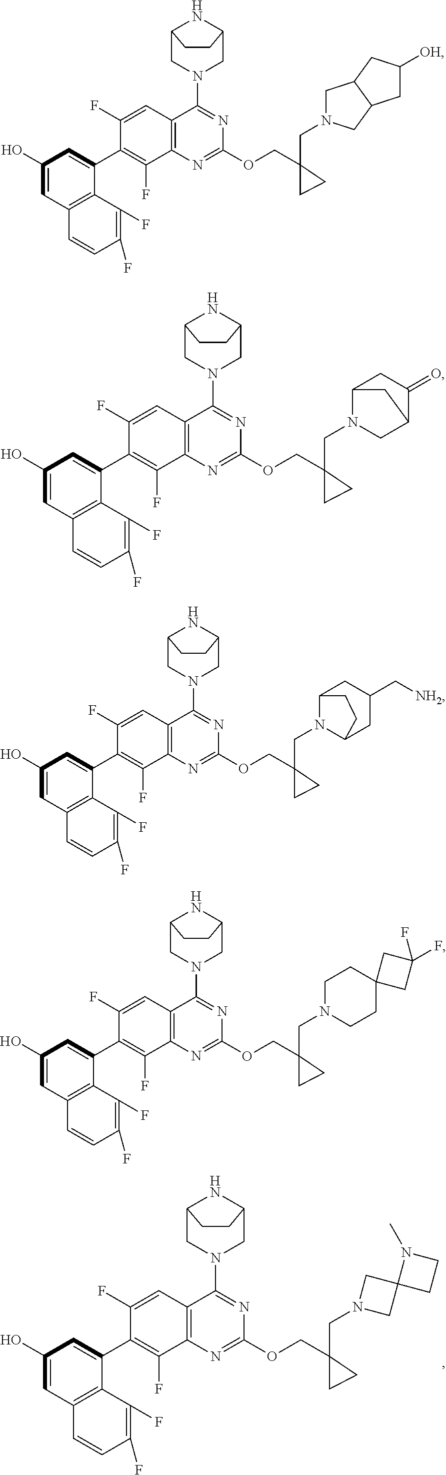

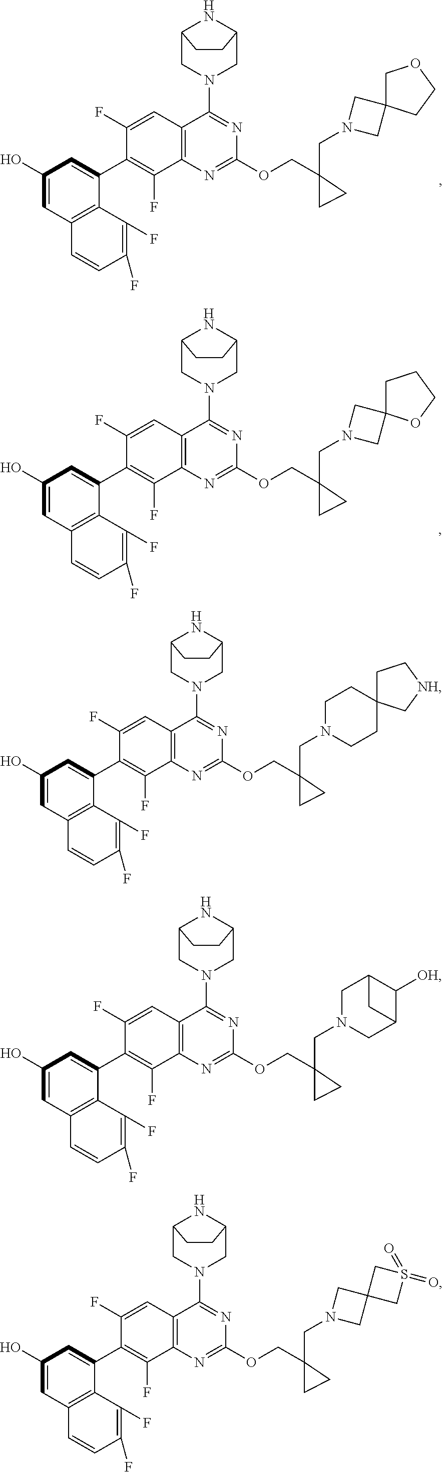

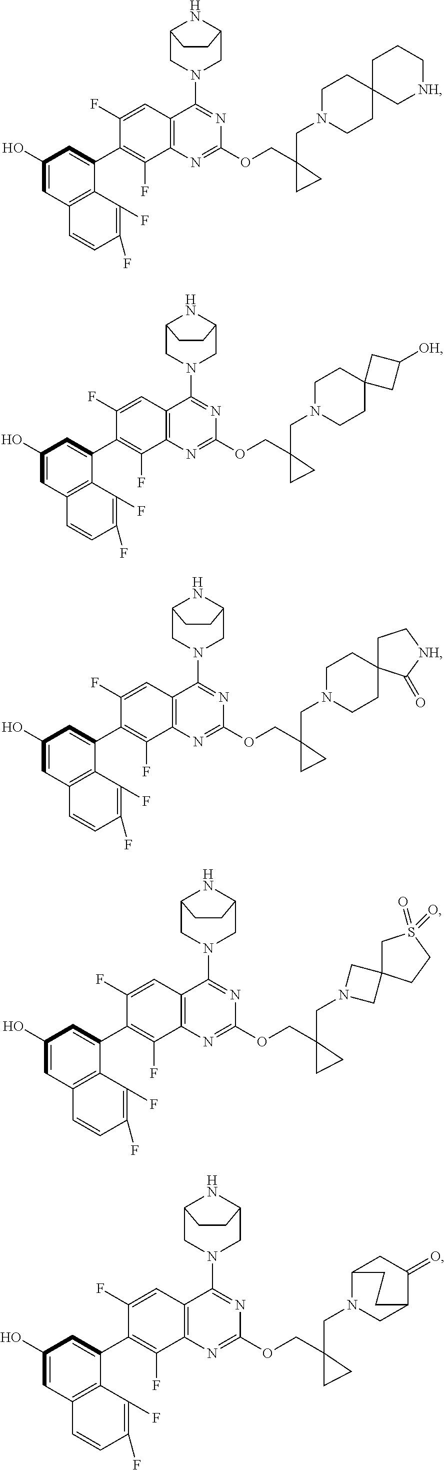

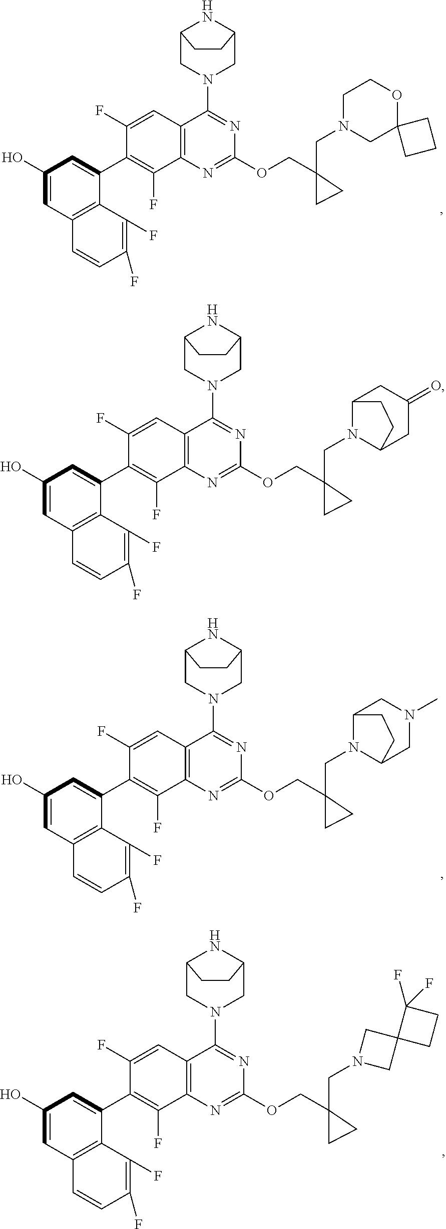

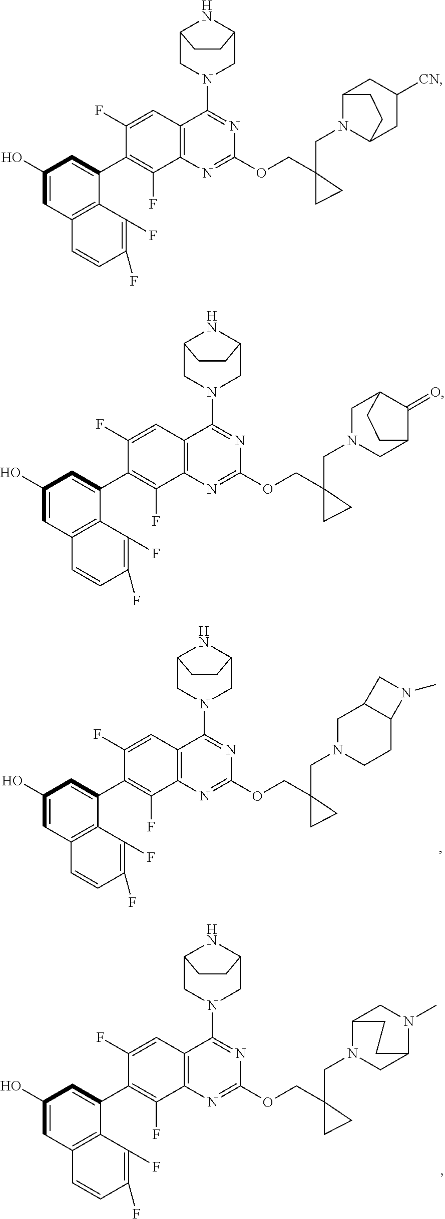

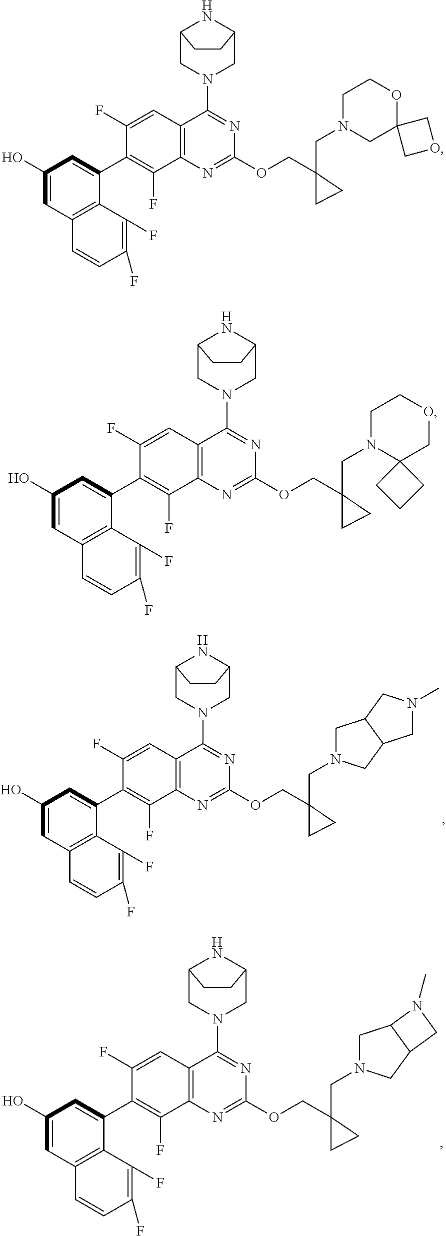

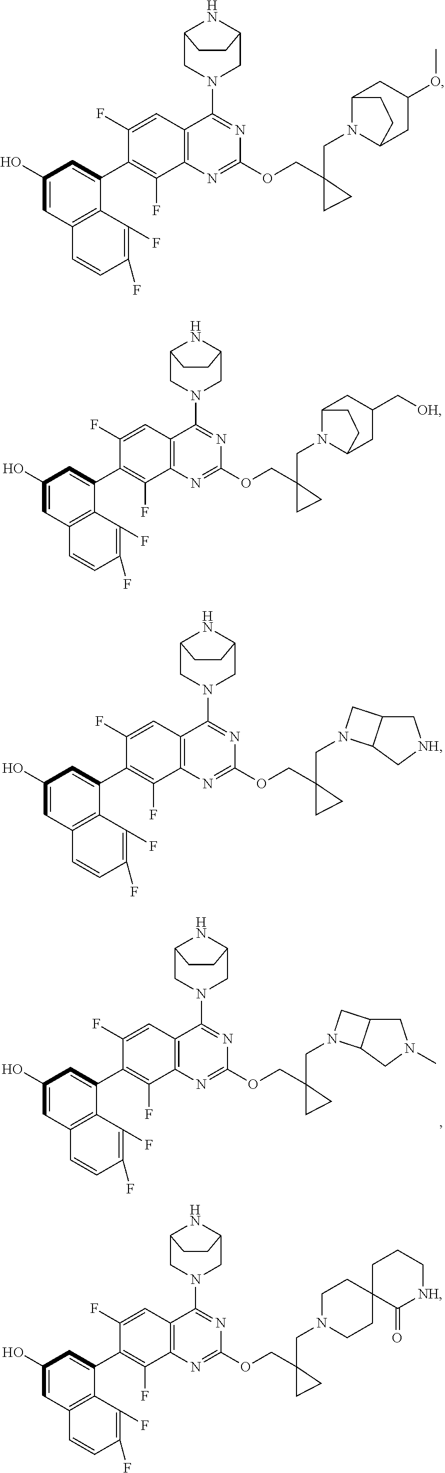

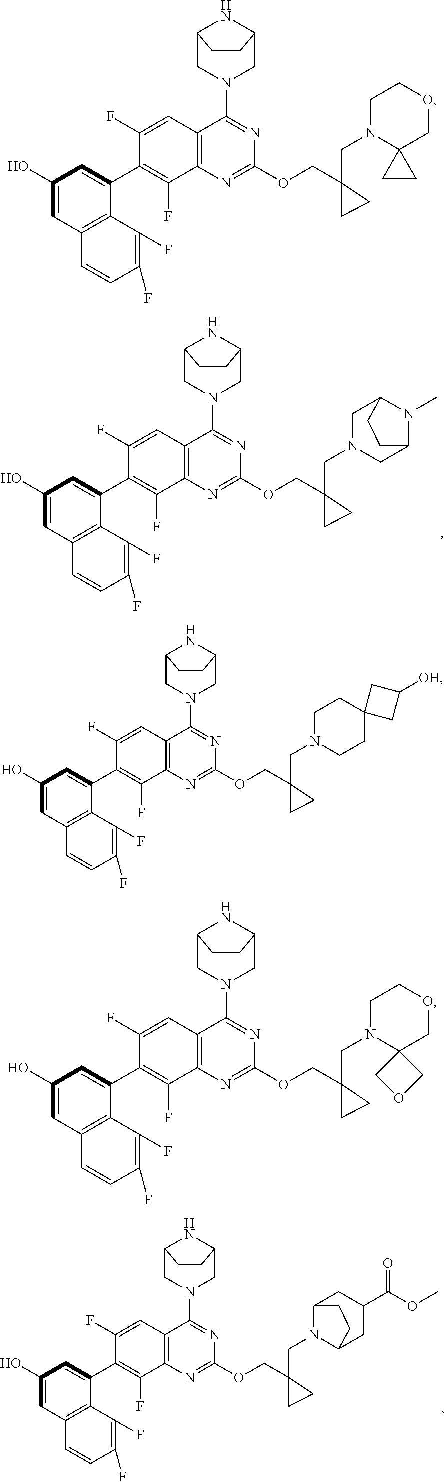

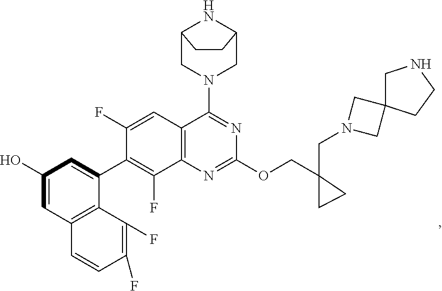

### Pharmacological Experiments

### 1. SOS1 Catalyzed Nucleotide Exchange Assay

GDP-loaded HIS-KRAS(G12D, aa 1-169) was pre-incubated with a compound in the presence of 10 nM GDPin a 384-well plate (Greiner) for 15 min, then purified SOS1 ExD(Flag tag, aa 564-1049), BODIPY™ FL GTP (Invitrogen) and MAb (monoclonal antibody) Anti 6HIS-Tb cryptate Gold (Cisbio) were added to the assay wells (Final concentration: 1.5 nM GDP-loaded HIS-KRAS(G12D), 5 nM GDP, 0.5 μM SOS1 ExD, 80 nM BODIPY™ FL GTP, 52.5 ng/mL MAb Anti 6HIS-Tb cryptate Gold) and incubated for 4 hours at 25° C. Wells containing same percent of DMSO served as vehicle control, and wells without KRAS served as low control. TR-FRET signals were read on Tecan Spark multimode microplate reader. The parameters were F486: Excitation 340 nm, Emission 486 nm, Lag time 100 μs, Integration time 200 μs; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalized between vehicle control and low control (% Activation=(TR-FRET ratio_{Compound treated}−TR-FRET ratio_{Low control})/(TR-FRET ratio_{Vehicle control}−TR-FRET ratio_{Low control})*100%). Then the data were analyzed by fitting a 4-parameter logistic model or calculating in Excel to calculate IC₅₀ values. The results of SOS1 catalyzed nucleotide exchange assay are shown in the following Table 1:

TABLE 1

| Compound | IC₅₀ (μM) |
|---|---|
| Compound 1 | 0.00541 |
| Compound 2 | 0.026 |
| Compound 2A | >0.1 |
| Compound 2B | 0.00124 |
| Compound 3 | 0.0371 |
| Compound 3A | >0.1 |
| Compound 3B | 0.00148 |
| Compound 4A | 0.244 |
| Compound 4B | 0.00031 |
| Compound 5 | 0.0555 |
| Compound 6 | 0.0662 |
| Compound 7 | 0.00627 |
| Compound 8 | 0.0168 |
| Compound 9 | 0.00777 |
| Compound 10 | 0.0121 |
| Compound 11 | 0.0316 |
| Compound 12 | 0.0142 |
| Compound 13 | 0.012 |
| Compound 13A | >0.1 |
| Compound 13B | 0.000874 |
| Compound 14 | 0.00436 |
| Compound 15 | 0.00606 |
| Compound 16 | 0.0273 |
| Compound 17 | 0.00199 |
| Compound 18 | 0.000975 |
| Compound 19 | 0.00227 |
| Compound 20 | 0.00133 |
| Compound 21 | 0.00194 |
| Compound 22 | 0.00139 |
| Compound 23 | >0.1 |
| Compound 24 | 0.00126 |
| Compound 24A | >0.1 |
| Compound 24B | 0.000912 |
| Compound 25 | 0.00148 |
| Compound 26 | 0.00258 |
| Compound 27 | 0.00961 |
| Compound 28 | 0.00176 |
| Compound 29 | 0.00350 |
| Compound 30 | 0.0286 |
| Compound 31 | 0.0232 |
| Compound 32 | 0.0355 |
| Compound 33 | 0.0316 |
| Compound 34 | 0.00307 |
| Compound 35 | 0.00217 |
| Compound 36 | 0.00210 |
| Compound 36A | >0.1 |
| Compound 36B | 0.00101 |
| Compound 37 | 0.00436 |
| Compound 38 | 0.00115 |
| Compound 39A | 0.0949 |
| Compound 39B | 0.00076 |
| Compound 40 | 0.00129 |
| Compound 41 | 0.00104 |
| Compound 42 | 0.00097 |
| Compound 43 | 0.00464 |
| Compound 44 | 0.0357 |
| Compound 45 | 0.0264 |
| Compound 46 | 0.00592 |
| Compound 47 | 0.00258 |
| Compound 48 | 0.00149 |
| Compound 49 | 0.00395 |
| Compound 50 | 0.00139 |
| Compound 51 | 0.00126 |
| Compound 52 | 0.00426 |
| Compound 53 | 0.00259 |
| Compound 54 | 0.011.4 |
| Compound 55 | 0.00137 |
| Compound 56 | 0.00205 |
| Compound 57 | 0.00681 |
| Compound 58 | 0.00204 |

2. GTP-KRAS and cRAF Interaction Assay

GppNp-loaded HIS-KRAS(G12D, aa 1-169) was pre-incubated with a compound in the presence of 200 μM GTP in a 384-well plate (Greiner) for 15 min, then cRAF RBD(GST tag, aa 50-132, CreativeBioMart), MAb Anti GST-d2 (Cisbio) and MAb Anti 6HIS-Tb cryptate Gold (Cisbio) were added to the assay wells (Final concentration: 2.0 nM GppNp-loaded HIS-KRAS(G12D), 100 μM GTP, 35 nM cRAF RBD, 1 μg/mL MAb Anti GST-d2, 52.5 ng/mL MAb Anti 6HIS-Tb cryptate Gold) and incubated for 2 hours at 25° C. Wells containing same percent of DMSO served as vehicle control, and wells without KRAS served as low control. HTRF signals were read on Tecan Spark multimode microplate reader and HTRF ratios were calculated under manufacturer's instructions. The percent of activation of compounds treated wells were normalized between vehicle control and low control (% Activation=(HTRF ratio_{Compound treated}−HTRF ratio_{Low control})/(HTRF ratio_{Vehicle control}−HTRF ratio_{Low control})*100%). Then the data were analyzed by fitting a 4-parameter logistic model or calculating in Excel to calculate IC₅₀ values. The results are shown in the Table 2:

TABLE 2

| Compound | IC₅₀ (μM) |
|---|---|
| Compound 1 | 0.247 |
| Compound 2 | 0.228 |
| Compound 2A | >10 |
| Compound 2B | 0.0413 |
| Compound 3 | 0.115 |
| Compound 3A | 9.078 |
| Compound 3B | 0.0173 |
| Compound 4A | 1.572 |
| Compound 4B | 0.00919 |
| Compound 5 | 0.31 |
| Compound 6 | 0.437 |
| Compound 7 | 0.0272 |
| Compound 8 | 0.307 |
| Compound 9 | 0.062 |
| Compound 10 | 0.0773 |
| Compound 11 | 0.0866 |
| Compound 12 | 0.0808 |
| Compound 13 | 64 |
| Compound 13A | >10 |
| Compound 13B | 0.0192 |
| Compound 14 | 0.0792 |
| Compound 15 | 0.157 |
| Compound 17 | 0.273 |
| Compound 18 | 0.0778 |
| Compound 19 | 0.0669 |
| Compound 20 | 0.0581 |
| Compound 21 | 0.0745 |
| Compound 22 | 0.0402 |
| Compound 23 | >10 |
| Compound 24 | 0.0252 |
| Compound 24A | >10 |
| Compound 24B | 0.0215 |
| Compound 25 | 0.051 |
| Compound 26 | 0.113 |
| Compound 27 | 0.19 |
| Compound 28 | 0.0525 |
| Compound 29 | 0.0716 |
| Compound 30 | 0.133 |
| Compound 31 | 0.108 |
| Compound 32 | 0.0931 |
| Compound 33 | 0.126 |
| Compound 34 | 0.0826 |
| Compound 35 | 0.157 |
| Compound 36 | 0.0351 |
| Compound 36A | 0.0202 |
| Compound 36B | 27331 |
| Compound 37 | 0.0501 |
| Compound 38 | 0.00582 |
| Compound 39A | 0.84 |
| Compound 39B | 0.00618 |
| Compound 40 | 0.00809 |
| Compound 41 | 0.00718 |
| Compound 42 | 0.00404 |
| Compound 43 | 0.436 |
| Compound 45 | 0.408 |
| Compound 46 | 0.242 |
| Compound 47 | 0.0921 |
| Compound 48 | 0.118 |
| Compound 49 | 0.0732 |
| Compound 50 | 0.0425 |
| Compound 51 | 0.0594 |
| Compound 52 | 0.286 |
| Compound 53 | 0.818 |
| Compound 55 | 0.0231 |
| Compound 56 | 0.0532 |
| Compound 57 | 0.439 |
| Compound 58 | 0.188 |

### 3. Phospho-ERK1/2(THR202/TYR204) HTRF Assay

AGS cells expressing KRAS G12D were cultured in F12K medium (Gibco) containing 10% fetal bovine serum (Gibco). The AGS cells in culture medium were seeded in 96-well plates at a concentration of 40,000 cells/well and then put in a 37° C./5% CO₂ cell incubator to incubate overnight. The next day, culture medium was removed and the compound diluted in assay medium (F12K, 0.1% FBS) was added in each well. After 2 hours incubation in a 37° C./5% CO₂ cell incubator, the assay medium in 96-well plates was removed, then 50 μL of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 μL/well HTRF® pre-mixed antibodies (Cisbio 64AERPEH). Incubate 4 hours at 25° C. and then read HTRF signals on Tecan Spark multimode microplate reader. The data were analyzed using a 4-parameter logistic model to calculate IC₅₀ values. The results are shown in the Table 3:

TABLE 3

| Compound | IC₅₀ (μM) |
|---|---|
| Compound 1 | 0.0583 |
| Compound 2B | 0.0303 |
| Compound 3 | 0.0955 |
| Compound 3B | 0.0202 |
| Compound 4 | 0.0388 |
| Compound 7 | 0.00353 |
| Compound 8 | 0.0212 |
| Compound 9 | 0.00259 |
| Compound 10 | 0.00618 |
| Compound 11 | 0.00949 |
| Compound 12 | 0.00194 |
| Compound 13A | 0.271 |
| Compound 13B | 0.00171 |
| Compound 14 | 0.182 |
| Compound 15 | 0.0689 |
| Compound 17 | 0.0563 |
| Compound 19 | 0.0198 |
| Compound 20 | 0.0392 |
| Compound 21 | 0.0135 |
| Compound 22 | 0.00695 |
| Compound 24 | 0.00248 |
| Compound 24B | 0.00767 |
| Compound 25 | 0.0147 |
| Compound 26 | 0.0563 |
| Compound 27 | 0.0193 |
| Compound 28 | 0.0262 |
| Compound 29 | 0.025 |
| Compound 30 | 0.0315 |
| Compound 31 | 0.0134 |
| Compound 32 | 0.0348 |
| Compound 33 | 0.0128 |
| Compound 34 | 0.0389 |
| Compound 35 | 0.0202 |
| Compound 36 | 0.00307 |
| Compound 36B | 0.00379 |
| Compound 37 | 0.0109 |
| Compound 45 | 0.125 |
| Compound 46 | 0.0231 |
| Compound 47 | 0.0314 |
| Compound 48 | 0.0386 |
| Compound 49 | 0.0464 |
| Compound 50 | 0.00939 |
| Compound 51 | 0.0121 |
| Compound 52 | 0.271 |
| Compound 53 | 0.0803 |
| Compound 55 | 0.00539 |
| Compound 56 | 0.00766 |
| Compound 57 | 0.0463 |

### 4. Cell Growth Inhibition Assay

AGS cultured in F12K medium (Gibco) containing 10% fetal bovine serum (Gibco). Cells in culture medium were plated in 96-well plates at a concentration of 500 cells/well (100 μL/well) and allowed to attach overnight.

AsPC-1 cultured in RPMI-1640 medium (Gibco) containing 10% fetal bovine serum (Gibco). Cells in culture medium were plated in 96-well plates at a concentration of 1000 cells/well (100 μL/well) and allowed to attach overnight.

The next day, compounds were diluted in culture medium and added to the plates. After 6 days incubation in a 37° C./5% CO₂ cell incubator, the cell viability was detected by CellTiter-Glo® Luminescent Cell Viability Assay kit (Promega). Luminescent signals were read on Tecan Spark multimode microplate reader and analyzed using a 4-parameter logistic model to calculate IC₅₀ values. The results are shown in the Table 4:

TABLE 4

| Compound | AsPC-1 IC₅₀ (μM) | AGS IC₅₀ (μM) |
|---|---|---|
| Compound 2B | 0.0135 | 0.000892 |
| Compound 3 | 0.0228 | 0.00676 |
| Compound 3B | 0.0144 | 0.00461 |
| Compound 5 | 0.0621 | 0.021 |
| Compound 7 | 0.0722 | 0.00566 |
| Compound 8 | 0.0553 | 0.0316 |
| Compound 9 | 0.138 | 0.0105 |
| Compound 10 | 0.0576 | 0.0111 |
| Compound 11 | 0.214 | 0.0157 |
| Compound 12 | 0.0832 | 0.00369 |
| Compound 13B | 0.0118 | 0.00277 |
| Compound 14 | 0.0265 | 0.0107 |
| Compound 15 | 0.0973 | 0.0139 |
| Compound 17 | 0.158 | 0.0118 |
| Compound 19 | 0.71 | 0.0129 |
| Compound 20 | 0.264 | 0.0233 |
| Compound 21 | 0.334 | 0.0163 |
| Compound 22 | 0.413 | 0.0197 |
| Compound 24 | 0.036 | 0.00366 |
| Compound 24B | 0.0601 | 0.00282 |
| Compound 25 | 0.0966 | 0.0132 |
| Compound 27 | 0.533 | 0.0293 |
| Compound 28 | 0.0986 | 0.00837 |
| Compound 30 | 0.0817 | 0.00977 |
| Compound 31 | 0.215 | 0.0262 |
| Compound 32 | 0.0671 | 0.00882 |
| Compound 33 | 0.151 | 0.0153 |
| Compound 34 | 0.208 | 0.0169 |
| Compound 35 | 0.0596 | 0.00843 |
| Compound 36 | 0.0774 | 0.00871 |
| Compound 36B | 0.0126 | 0.000688 |
| Compound 37 | 0.0899 | 0.00648 |
| Compound 45 | 0.653 | 0.0742 |
| Compound 46 | 0.488 | 0.0554 |
| Compound 47 | 0.227 | 0.029 |
| Compound 48 | 0.315 | 0.0364 |
| Compound 50 | 0.07 | 0.00349 |
| Compound 51 | 0.066 | 0.00125 |
| Compound 52 | 0.093 | 0.0324 |
| Compound 55 | 0.077 | 0.00771 |
| Compound 56 | 0.182 | 0.0149 |
| Compound 58 | 0.091 | 0.034 |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

1-114. (canceled)

115. A compound of formula (I), a stereoisomer thereof, an atropisomer thereof, a deuterated derivative thereof, a tautomer thereof, a prodrug thereof, or a pharmaceutically acceptable salt thereof: 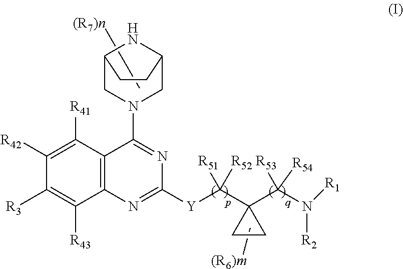
Wherein,
Y is selected from a bond, O, NR₅₅, S, S═O, or S(═O)₂;
R₁ and R₂ together with the nitrogen atom to which they are both attached form 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-20 membered monocyclic heterocyclic ring; said 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-20 membered monocyclic heterocyclic ring optionally further contains ring members selected from —O—, —S—, —S(═O)—, —S(═O)₂—, —C(═O)—, —NH—, —CH₂—, —CHF—, —CF₂—, —C(═O)NH—, —NHC(═O)—, —S(═O)NH—, —NHS(═O)—, —S(═O)₂NH— or —NHS(═O)₂—; said 5-20 membered spirocyclic heterocyclic ring, 5-20 membered fused heterocyclic ring, 5-20 membered bridged heterocyclic ring, 4 membered monocyclic heterocyclic ring, 7 membered monocyclic heterocyclic ring, or 8-15 membered monocyclic heterocyclic is independently optionally substituted with one or more R_{S};
R_{S} at each occurrence is independently selected from halogen —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein said —C₁₋₆alkyl, —C₁₋₆haloalkyl, —C₁₋₆haloalkoxy, —C₂₋₆alkenyl, —C₂₋₆alkynyl, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₃ is selected from phenyl, naphthyl, 5 membered heteroaryl, 6 membered heteroaryl, 8 membered heteroaryl, 9 membered heteroaryl or 10 membered heteroaryl; each of which is independently optionally substituted with one or more R₃₁;
R₃₁ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₄₁, R₄₂ or R₄₃ at each occurrence is independently selected from hydrogen, halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₅₁, R₅₂, R₅₃, R₅₄ or R₅₅ at each occurrence is independently selected from hydrogen, halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₆ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
R₇ at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein each of which is independently optionally substituted with 1, 2, 3, 4, 5 or 6 substituents selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
m, n, p or q is independently selected from 0, 1, 2, 3, 4, 5, 6, 7, 8, 9 or 10;
said heterocyclyl, heterocyclic, or heteroaryl at each occurrence contains 1, 2, 3, 4, or 5 ring members selected from N, O, S, S(═O) or S(═O)₂.

116. The compound according to claim 115, wherein, the compound of formula (I) is selected from any one of formula (I-A) to formula (I-F): 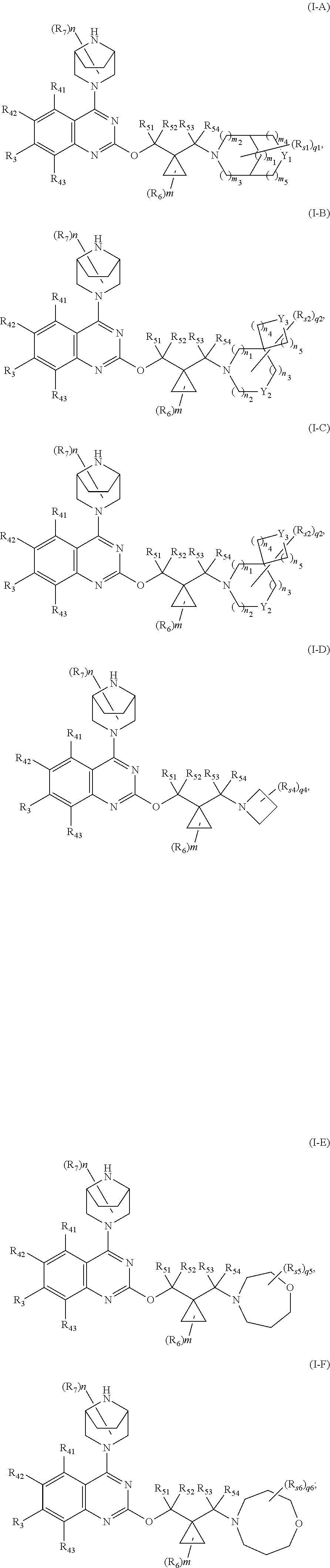
Y₁ is selected from —O—, —S—, —S(═O)—, —S(═O)₂—, —C(═O)—, —NH—, —CH₂—, —CHF— or —CF₂—;
m₁, m₂, m₃, m₄ or m₅ is independently selected from 0, 1, 2, 3, 4, 5 or 6;
Y₂ is selected from —O—, —S—, —S(═O)—, —S(═O)₂—, —C(═O)—, —NH—, —CH₂—, —CHF— or —CF₂—;
Y₃ are each independently selected from —O—, —S—, —S(═O)—, —S(═O)₂—, —C(═O)—, —NH—, —CH₂—, —CHF—, —CF₂—, —C(═O)NH—, —NHC(═O)—, —S(═O)NH—, —NHS(═O)—, —S(═O)₂NH— or —NHS(═O)₂—;
n₁, n₂, n₃, n₄ or n₅ is independently selected from 0, 1, 2, 3, 4, 5 or 6;
Ring A is selected from a 3-7 membered carbocyclic ring; 3-7 membered heterocyclic including 1, 2 or 3 ring members selected from —O—, —S—, —S(═O)—, —S(═O)₂—, —C(═O), —NH—, —CH₂—, —CHF— or —CF₂—; phenyl ring; or a 5-6 membered heteroaryl ring including 1, 2 or 3 ring members selected from N, O or S;
Z₁ is selected from C, CH or N;
r₁ or r₂ is independently selected from 0, 1, 2, 3, 4, 5 or 6;
R_{S1}, R_{S2}, R_{S3}, R_{S4}, R_{S5} or R_{S6} at each occurrence is independently selected from halogen, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl, wherein said —C₁₋₆alkyl, —C₁₋₆haloalkyl, —C₁₋₆haloalkoxy, —C₂₋₆alkenyl, —C₂₋₆alkynyl, 3-6 membered cycloalkyl, 3-6 membered heterocyclyl, 6-10 membered aryl or 5-10 membered heteroaryl is independently optionally substituted with 1, 2, 3, 4, 5 or 6 substituents selected from —F, —Cl, —Br, —C₁₋₆alkyl, —C₁₋₆haloalkyl, —C₁₋₆haloalkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered heterocyclyl, 6-10 membered aryl or 5-10 membered heteroaryl;
q₁, q₂, q₃, q₄, q₅, or q₆ at each occurrence is independently selected from 0, 1, 2, 3, 4, 5, 6, 7, 8, 9 or 10;
said heterocyclyl, heterocyclic, or heteroaryl at each occurrence contains 1, 2, 3, 4, or 5 ring members selected from N, O, S, S(═O) or S(═O)₂.

117. The compound according to claim 116, wherein, the compound of formula (I-A) is selected from any one of formula (I-A1) to formula (I-A6): 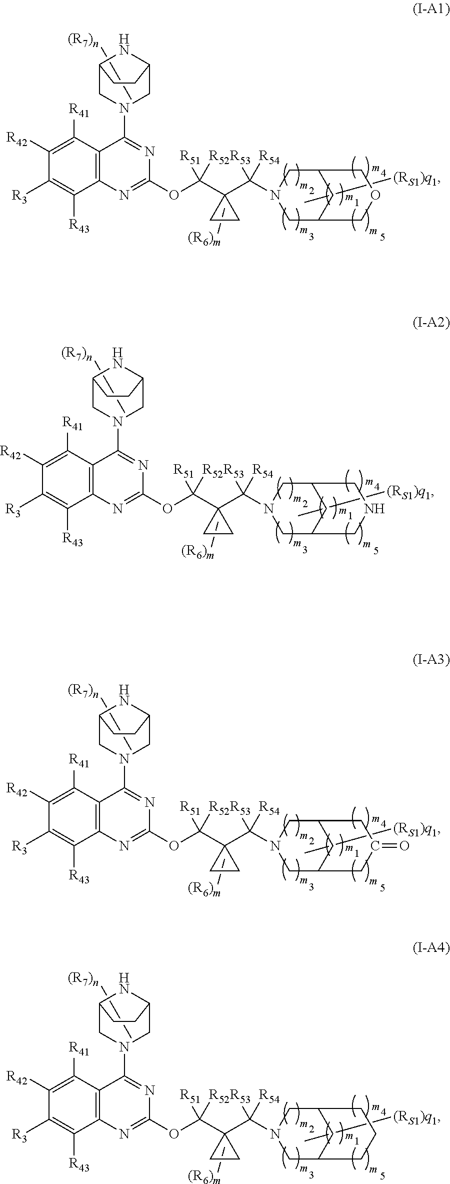 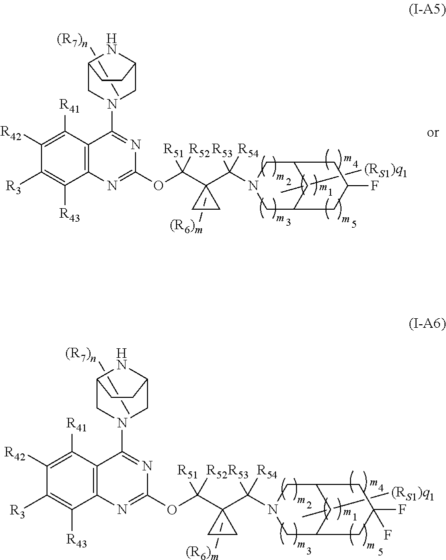
wherein,
the moiety of 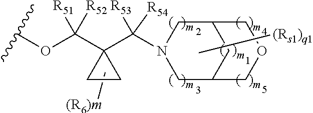
in the formula (I-A1) is selected from 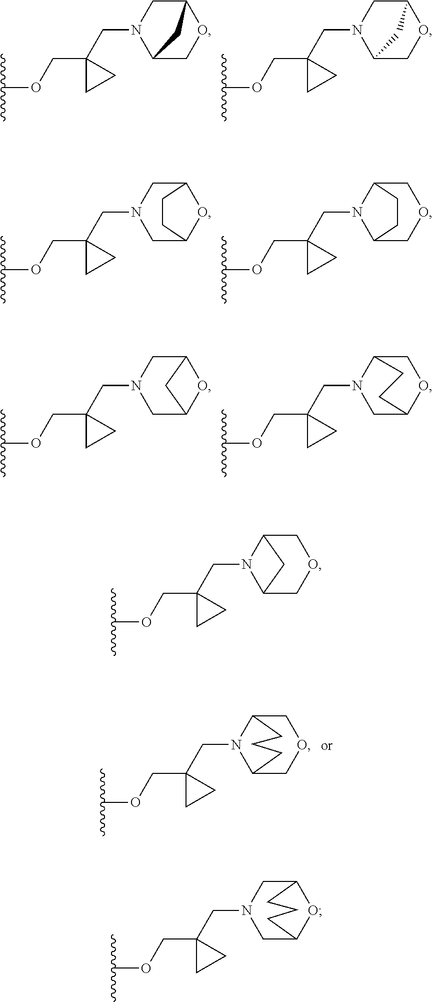
the moiety of 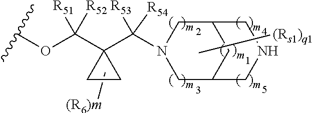
in the formula (I-A2) is selected from 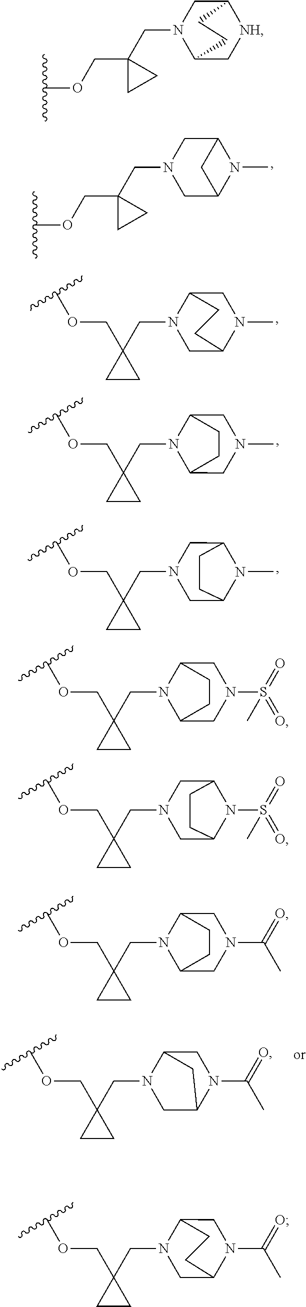
the moiety of 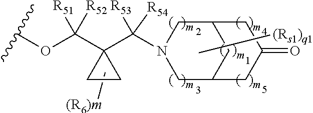
in the formula (I-A3) is selected from 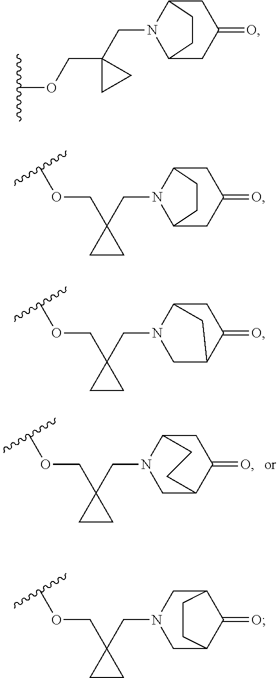 [SMILES: *OCC1(CN2C3CCC2CC(=O)C3)CC1 ; *OCC1(CN2C3CCC2CC(=O)C3)CC1 ; *OCC1(CN2CC3CC2CC3=O)CC1 ; *OCC1(CN2CC3CCC2CC3=O)CC1 ; *OCC1(CN2CC3CCC(C2)C3=O)CC1]
the moiety of 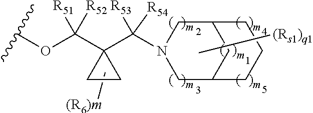
in the formula (I-A4) is selected from 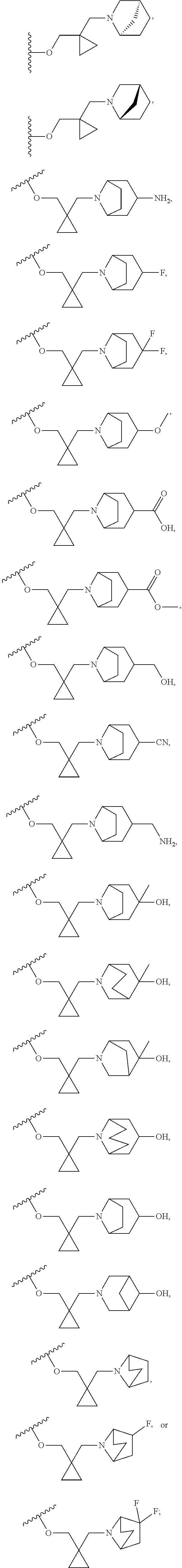
the moiety of 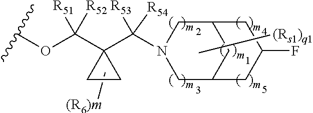
in the formula (I-A5) is selected from 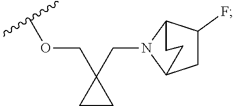 [SMILES: *OCC1(CN2C3CCC2C(F)C3)CC1]
and
the moiety of 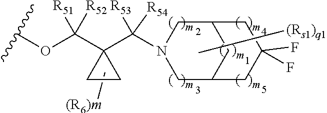
in the formula (I-A6) is selected from

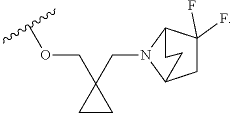 [SMILES: *OCC1(CN2C3CCC2C(F)(F)C3)CC1]

118. The compound according to claim 116, wherein, the compound of formula (I-B) is selected from any one of formula (I-B1) to (I-B9): 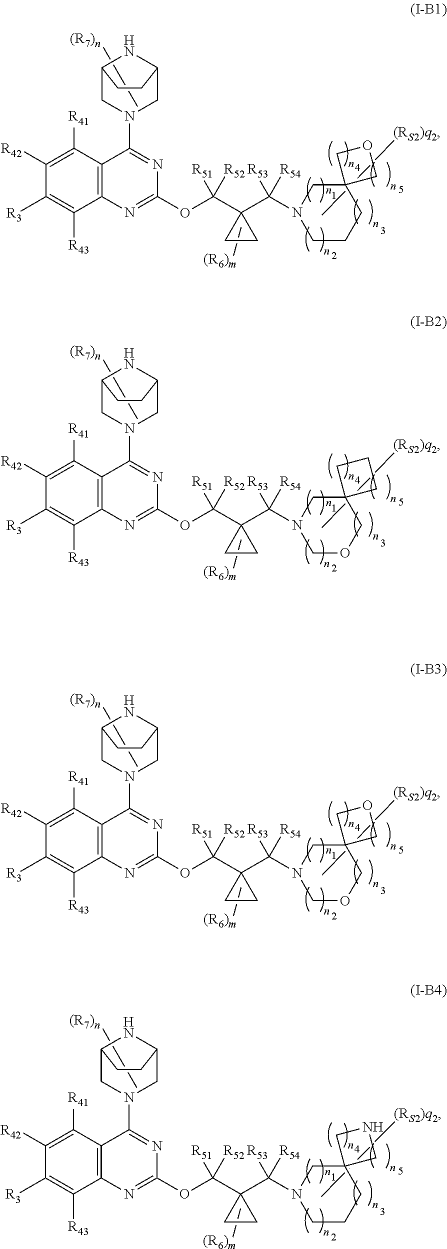 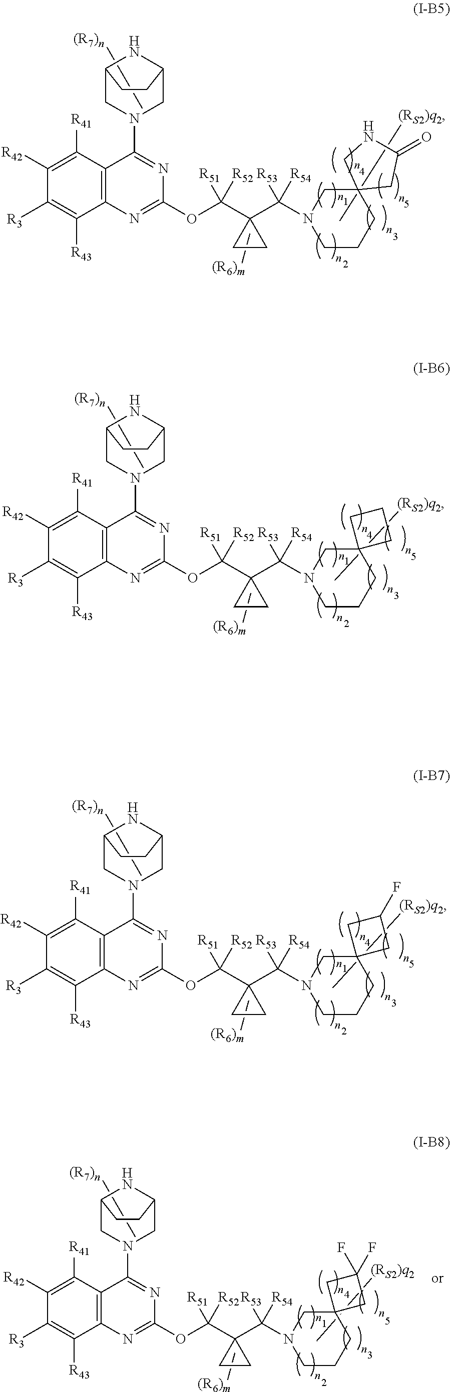 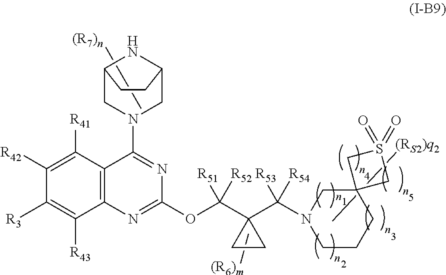
wherein,
the moiety of 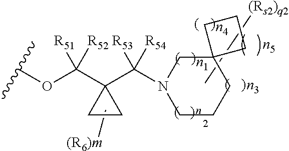
in formula (I-B1) is selected from 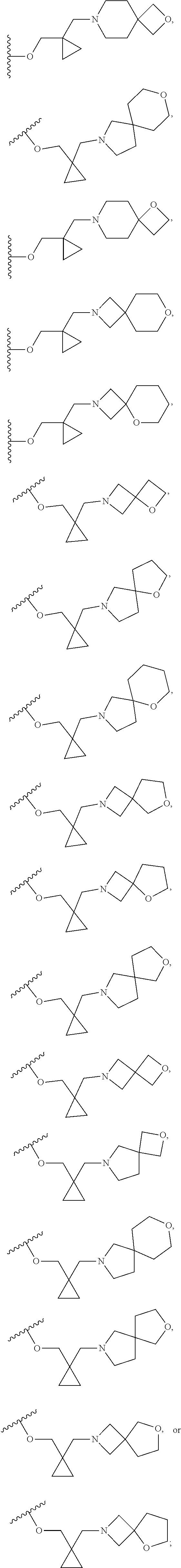
the moiety of 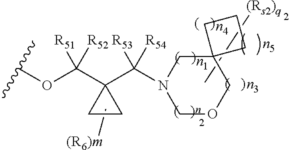
in the formula (I-B2) is selected from 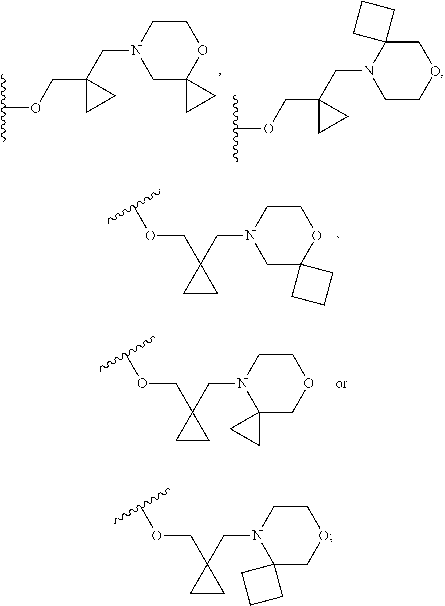
the moiety of 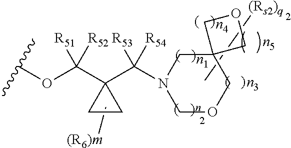
in the formula (I-B3) is selected from 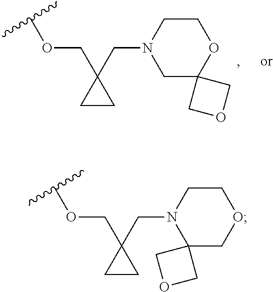 [SMILES: *OCC1(CN2CCOC3(COC3)C2)CC1 ; *OCC1(CN2CCOCC23COC3)CC1]
the moiety of 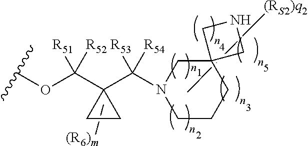
in the formula (I-B4) is selected from 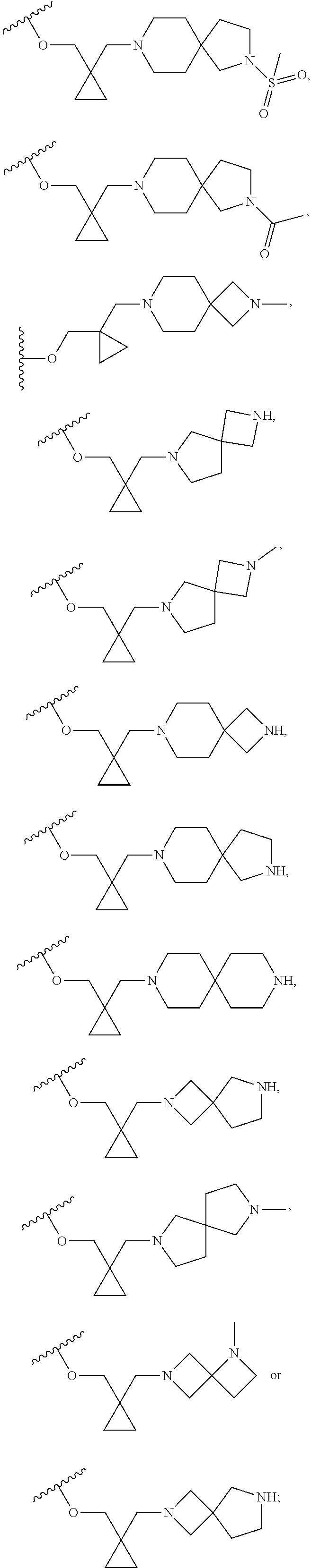
the moiety of 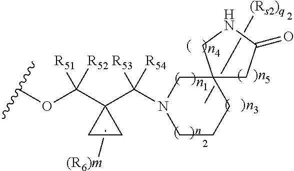
in the formula (I-B5) is selected from 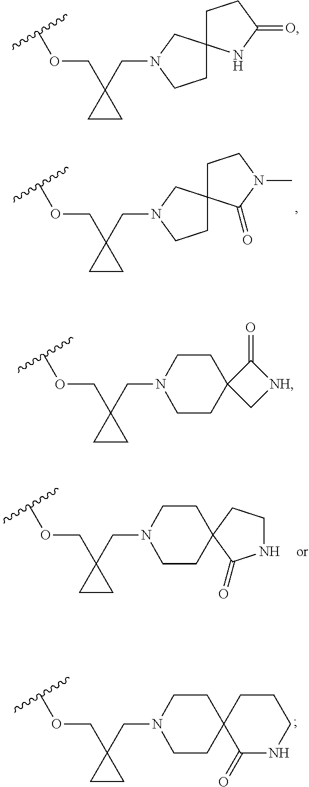
wherein, the moiety of 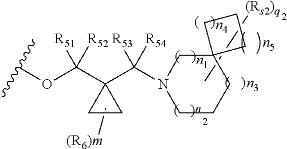
in the formula (I-B6) is selected from 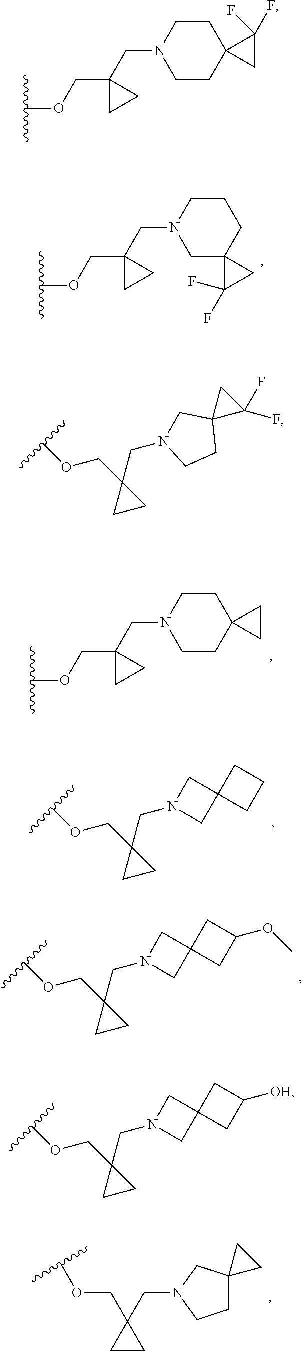 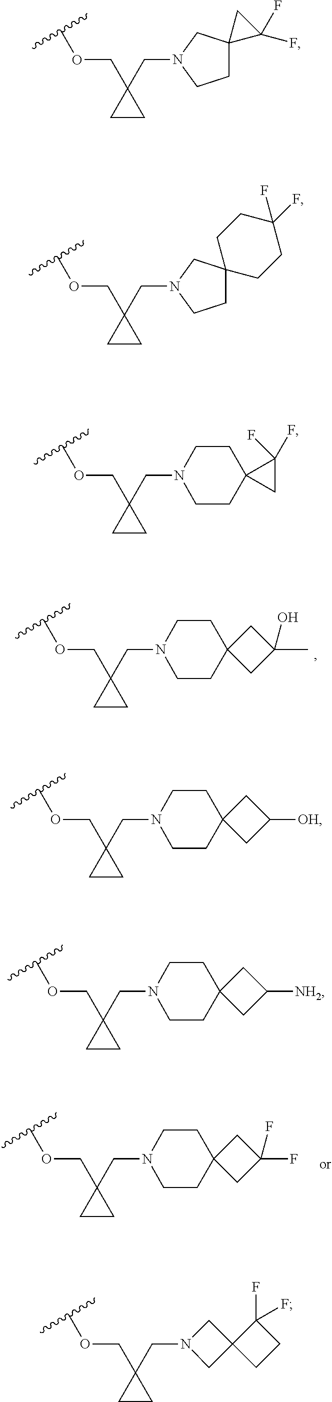
the moiety of 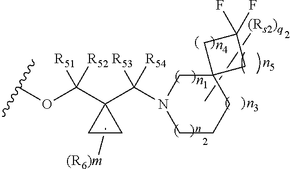
in the formula (I-B8) is selected from 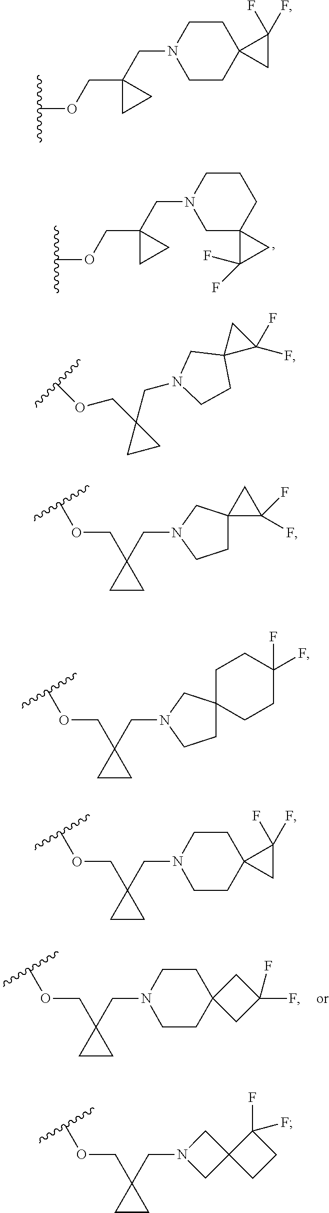
and
wherein, the moiety of 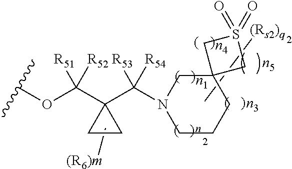
in the formula (I-B9) is selected from

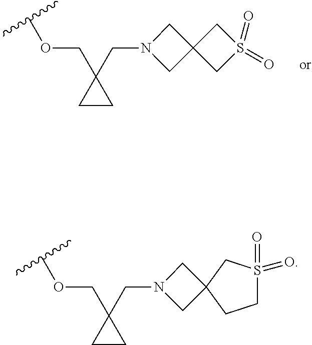 [SMILES: *OCC1(CN2CC3(C2)CS(=O)(=O)C3)CC1 ; *OCC1(CN2CC3(CCS(=O)(=O)C3)C2)CC1]

119. The compound according to claim 116, wherein, the compound of formula (I-C) is selected from any one of formula (I-C1-1) to formula (I-C1-5): 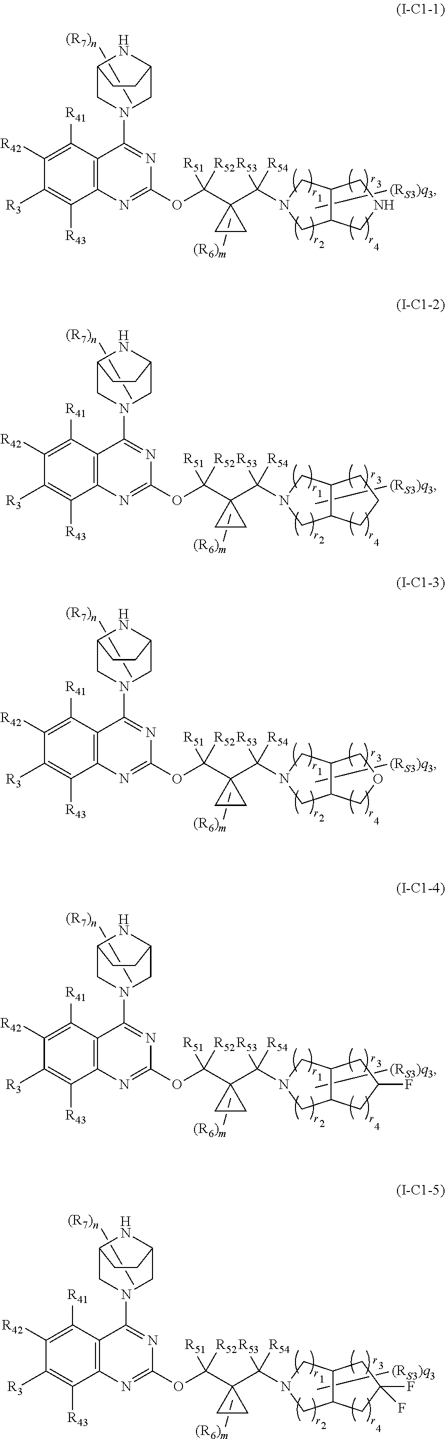
wherein,
the moiety of 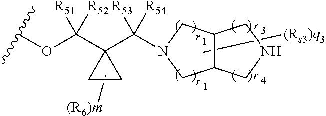
in formula (I-C1-1) is selected from 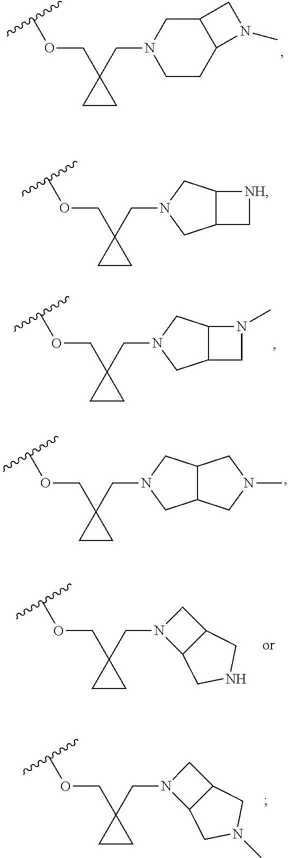 [SMILES: *OCC1(CN2CCC3C(C2)CN3C)CC1 ; *OCC1(CN2CC3CNC3C2)CC1 ; *OCC1(CN2CC3CN(C)C3C2)CC1 ; *OCC1(CN2CC3CN(C)CC3C2)CC1 ; *OCC1(CN2CC3CNCC32)CC1 ; *OCC1(CN2CC3CN(C)CC32)CC1]
the moiety of 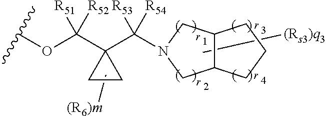
in formula (I-C1-2) is selected from 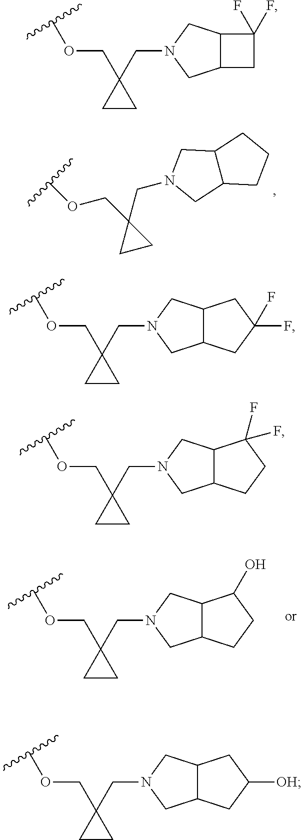 [SMILES: *OCC1(CN2CC3CC(F)(F)C3C2)CC1 ; *OCC1(CN2CC3CCCC3C2)CC1 ; *OCC1(CN2CC3CC(F)(F)CC3C2)CC1 ; *OCC1(CN2CC3CCC(F)(F)C3C2)CC1 ; *OCC1(CN2CC3CCC(O)C3C2)CC1 ; *OCC1(CN2CC3CC(O)CC3C2)CC1]
the moiety of 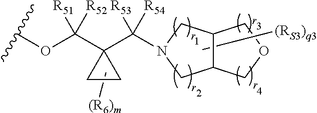
in formula (I-C1-3) is selected from 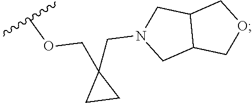 [SMILES: *OCC1(CN2CC3COCC3C2)CC1]
and
the moiety of 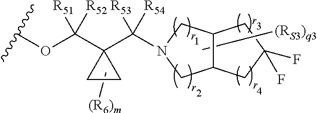
in formula (I-C1-5) is selected from 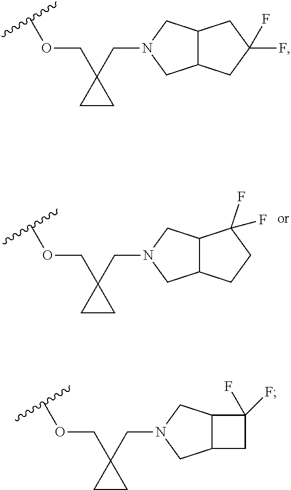 [SMILES: *OCC1(CN2CC3CC(F)(F)CC3C2)CC1 ; *OCC1(CN2CC3CCC(F)(F)C3C2)CC1 ; "or" ; *OCC1(CN2CC3CC(F)(F)C3C2)CC1]
or
the compound of formula (I-C) is selected from formula (I-C2-1) or formula (I-C2-2): 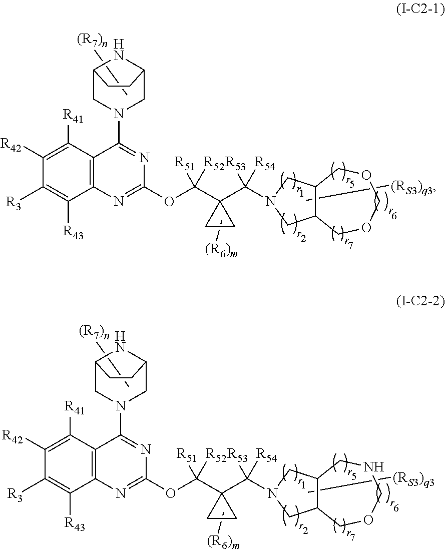
wherein,
the moiety of 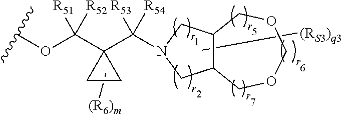
in formula (I-C2-1) is selected from 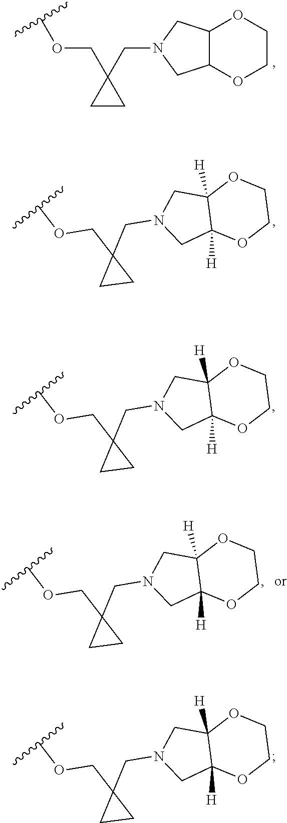 [SMILES: *OCC1(CN2CC3OCCOC3C2)CC1 ; *OCC1(CN2C[C@@H]3OCCO[C@@H]3C2)CC1 ; *OCC1(CN2C[C@@H]3OCCO[C@H]3C2)CC1 ; *OCC1(CN2C[C@H]3OCCO[C@@H]3C2)CC1 ; *OCC1(CN2C[C@@H]3OCCO[C@@H]3C2)CC1]
and
the moiety of 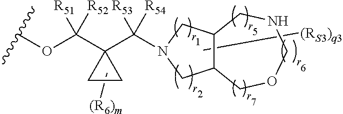
in the formula (I-C2-2) is selected from 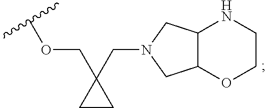 [SMILES: *OCC1(CN2CC3NCCOC3C2)CC1]
or
the compound of formula (I-C) is selected from formula (I-C3-1), formula (I-C3-2), or formula (I-C3-3): 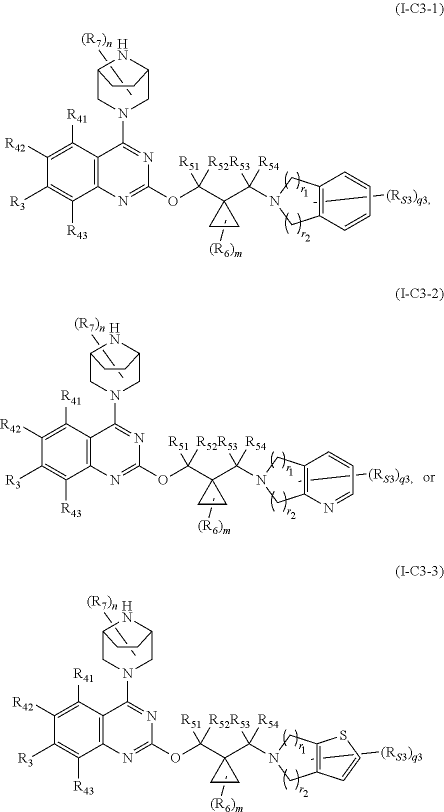
wherein,
r₁ is selected from 1 or 2; r₂ is selected from 1;
the moiety of 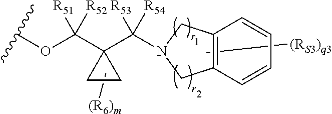
in formula (I-C3-1) is selected from 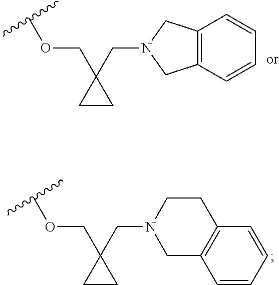 [SMILES: *OCC1(CN2Cc3ccccc3C2)CC1 ; *OCC1(CN2CCc3ccccc3C2)CC1]
the moiety of 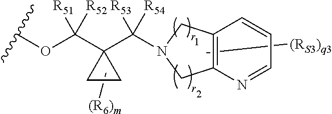
in formula (I-C3-2) is selected from 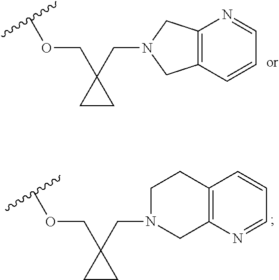 [SMILES: *OCC1(CN2Cc3cccnc3C2)CC1 ; *OCC1(CN2CCc3cccnc3C2)CC1]
and
the moiety of 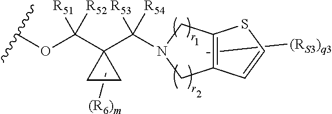
in formula (I-C3-3) is selected from 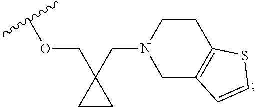 [SMILES: *OCC1(CN2CCc3sccc3C2)CC1]
or
the compound of formula (I-C) is selected from formula (I-C4-1), 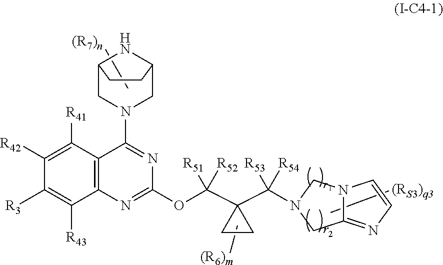
wherein,
r₁ is selected from 1 or 2; r₂ is selected from 1;
the moiety of 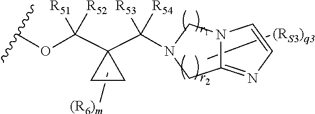
in formula (I-C4-1) is selected from

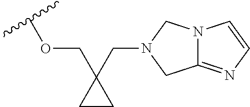 [SMILES: *OCC1(CN2Cc3nccn3C2)CC1]

[symbol]

120. The compound according to claim 116, wherein, the moiety of 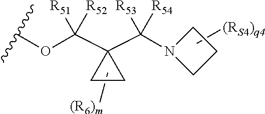
in the formula (I-D) is independently selected from

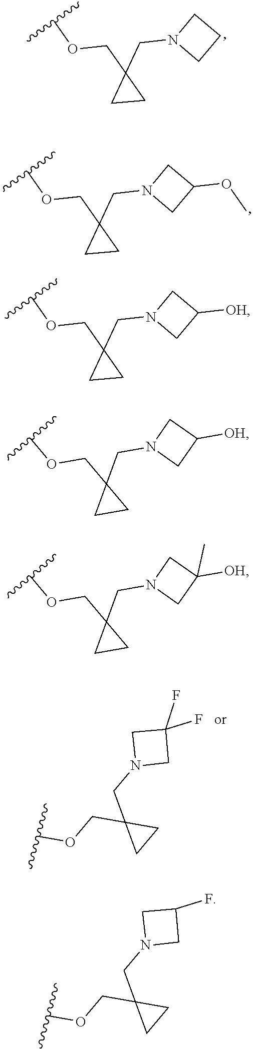

121. The compound according to claim 116, wherein, the moiety of 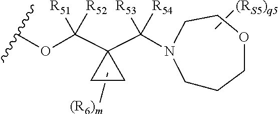
in formula (I-E) is selected from

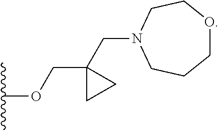 [SMILES: *OCC1(CN2CCCOCC2)CC1]

122. The compound according to claim 116, wherein, the moiety of 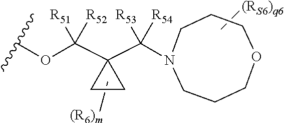
in formula (I-F) is selected from

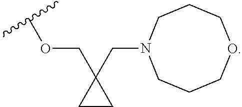 [SMILES: *OCC1(CN2CCCOCCC2)CC1]

123. The compound according to claim 115, wherein, R₃ is selected from: 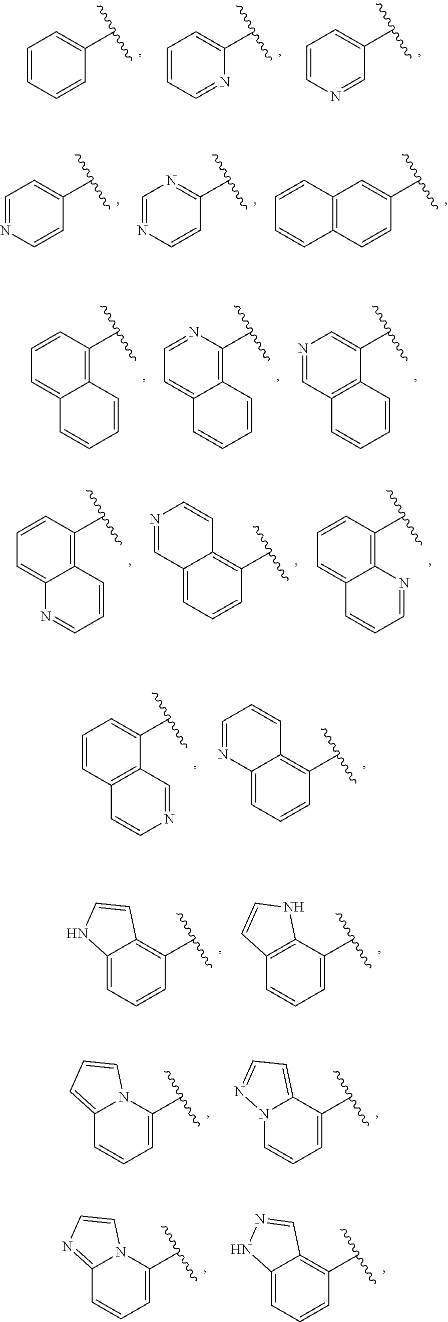 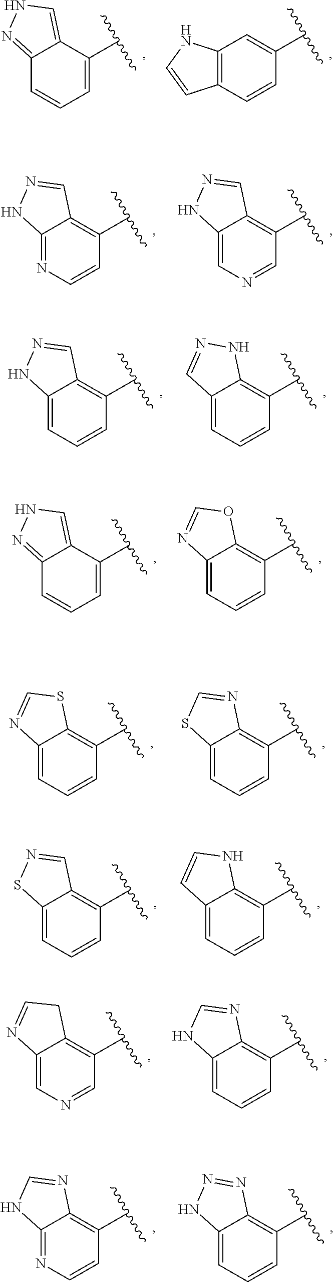 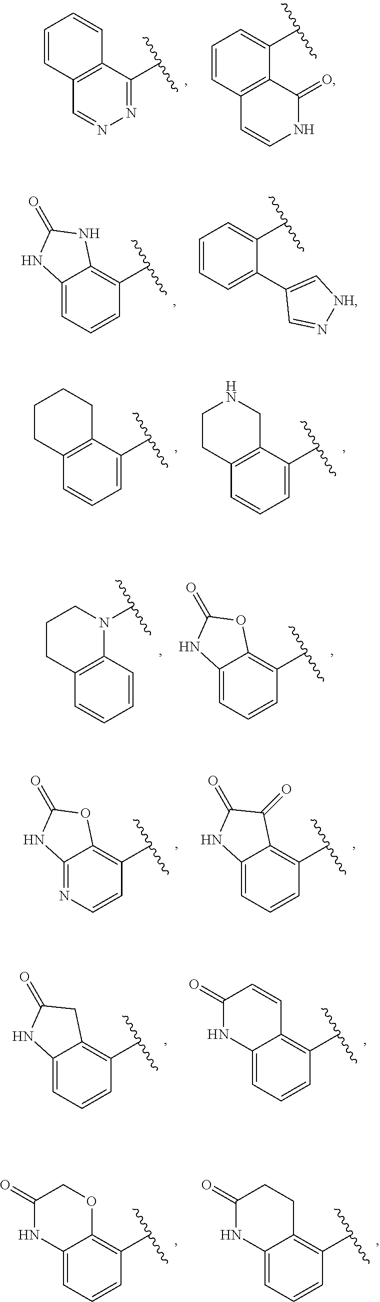 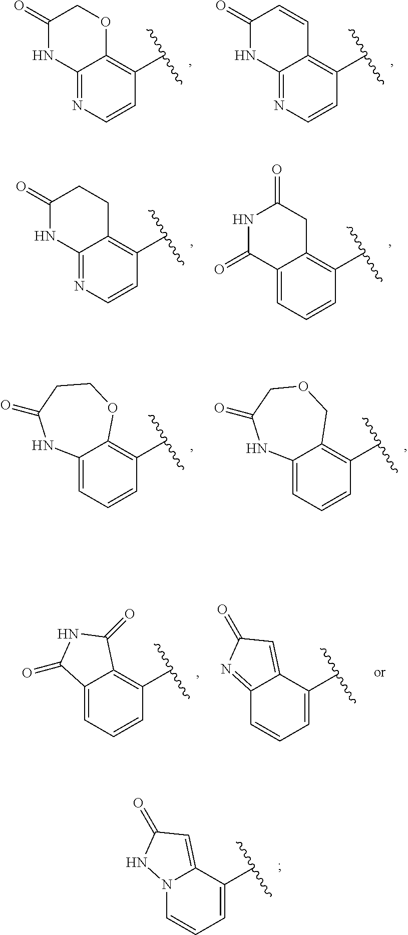
Each of which is independently optionally substituted with 1, 2, 3, 4, 5 or 6 R₃₁.

124. The compound according to claim 115, wherein, R₃ is selected from

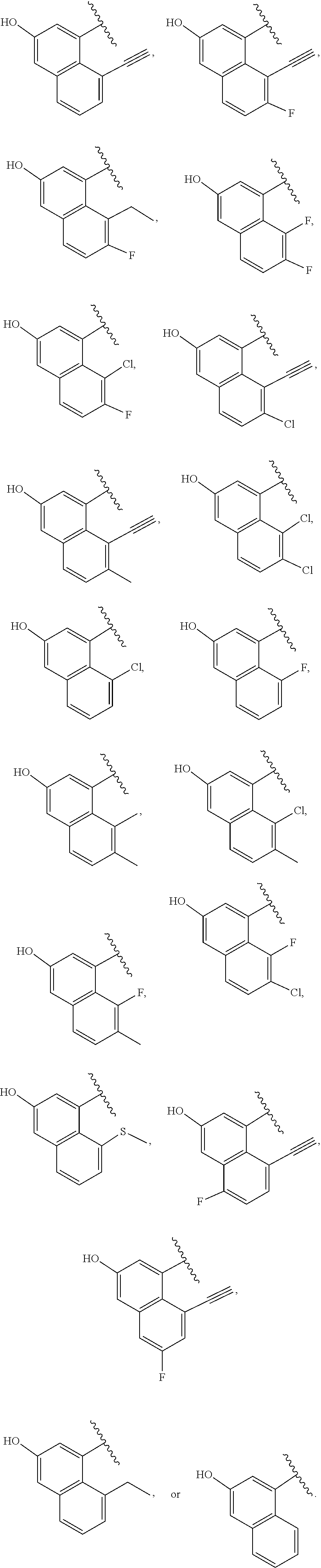

125. The compound according to claim 124, wherein, R₃ is selected from

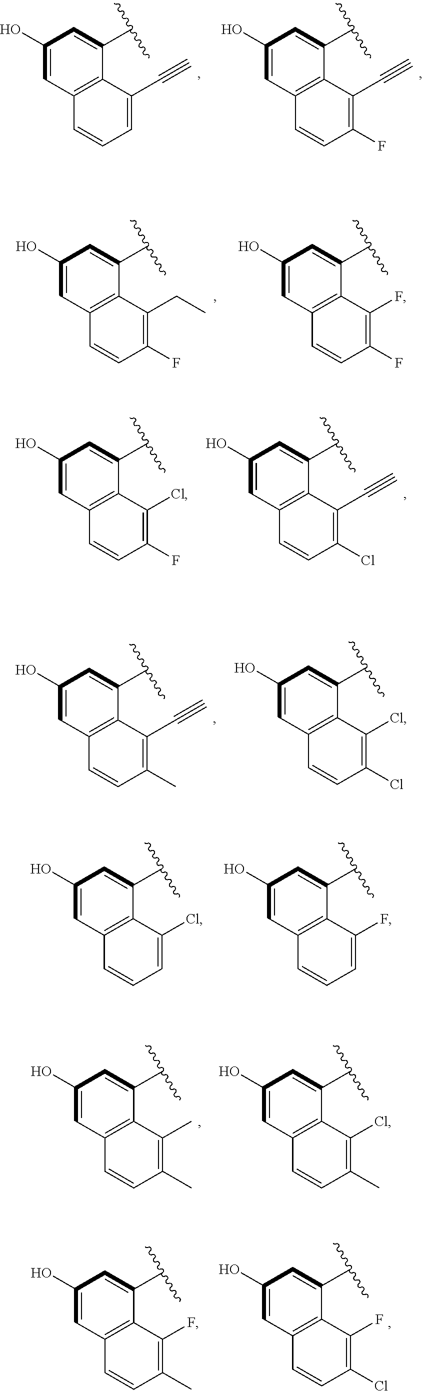

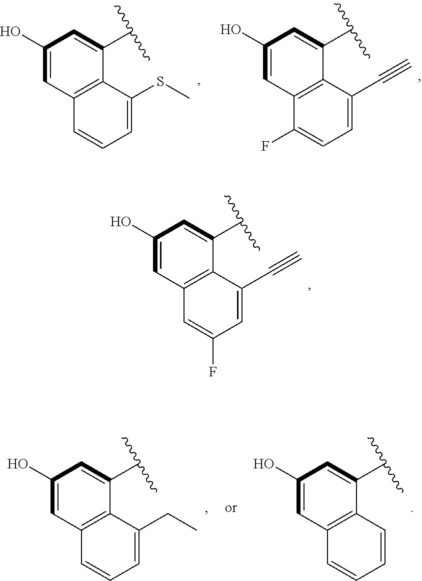

126. The compound according to claim 115, wherein,
the compound of formula (I) is selected from any one of the following formulas:

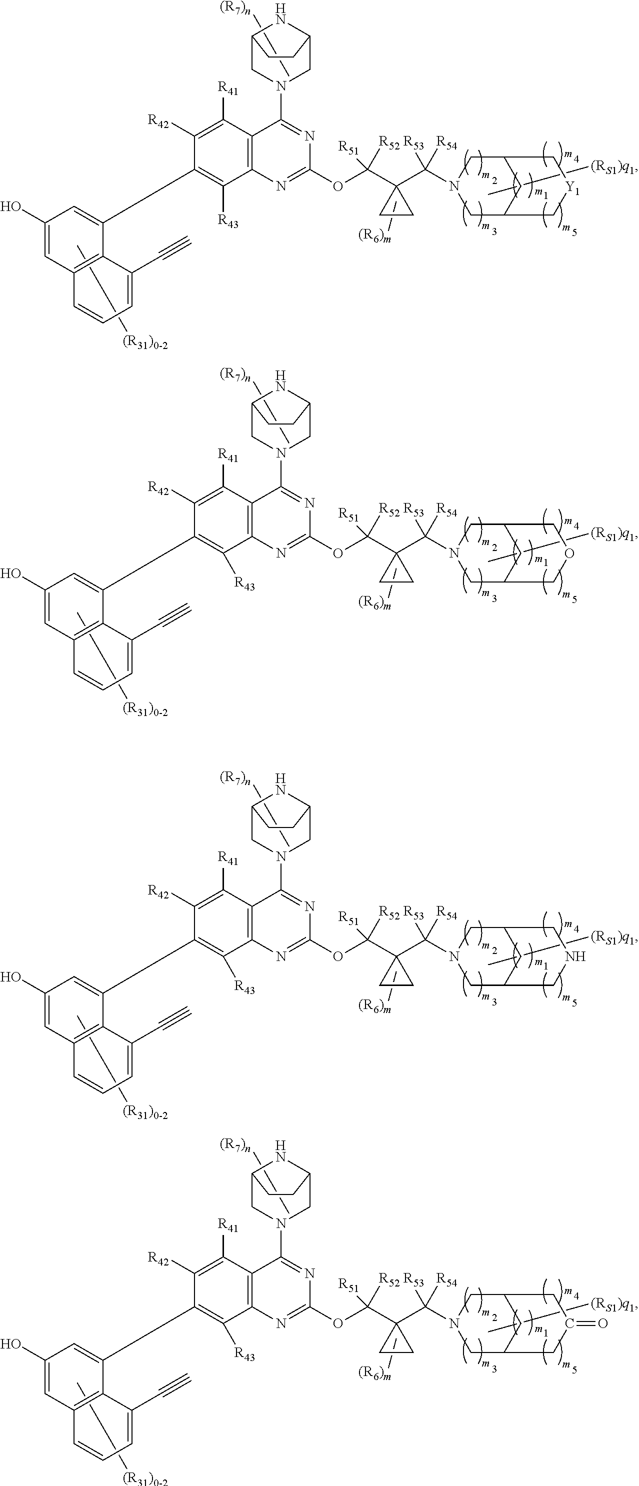

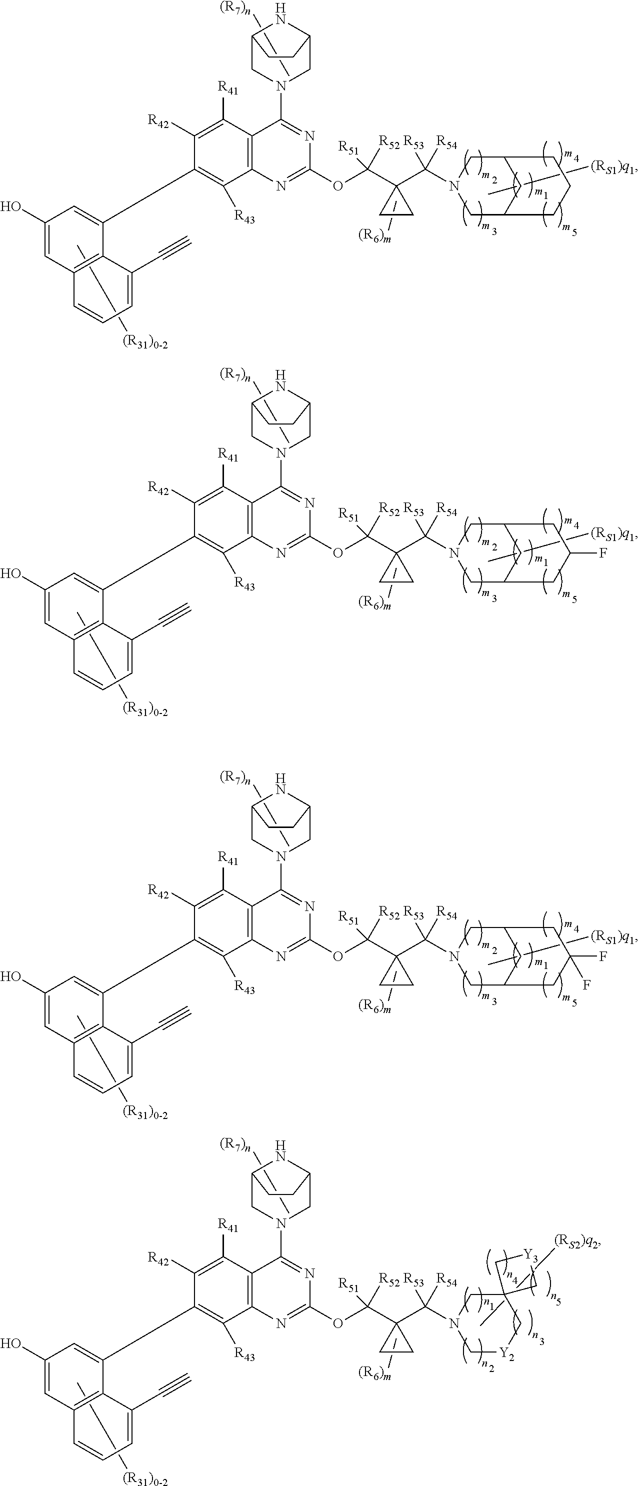

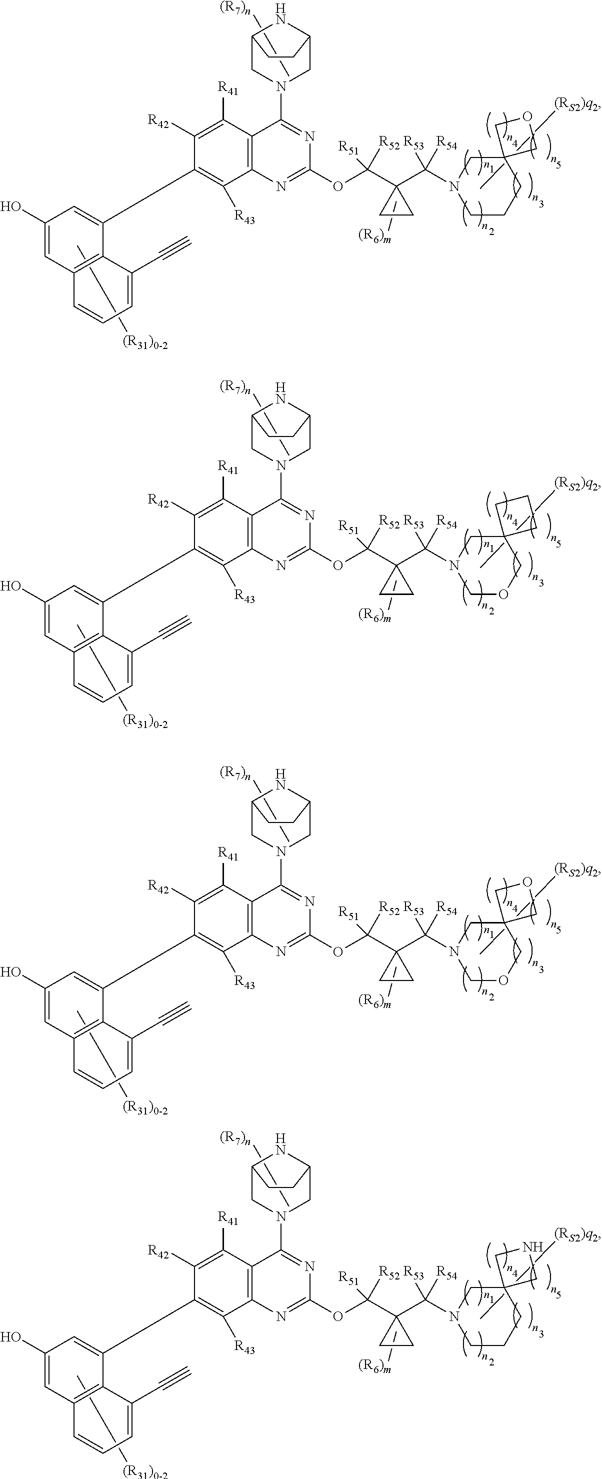

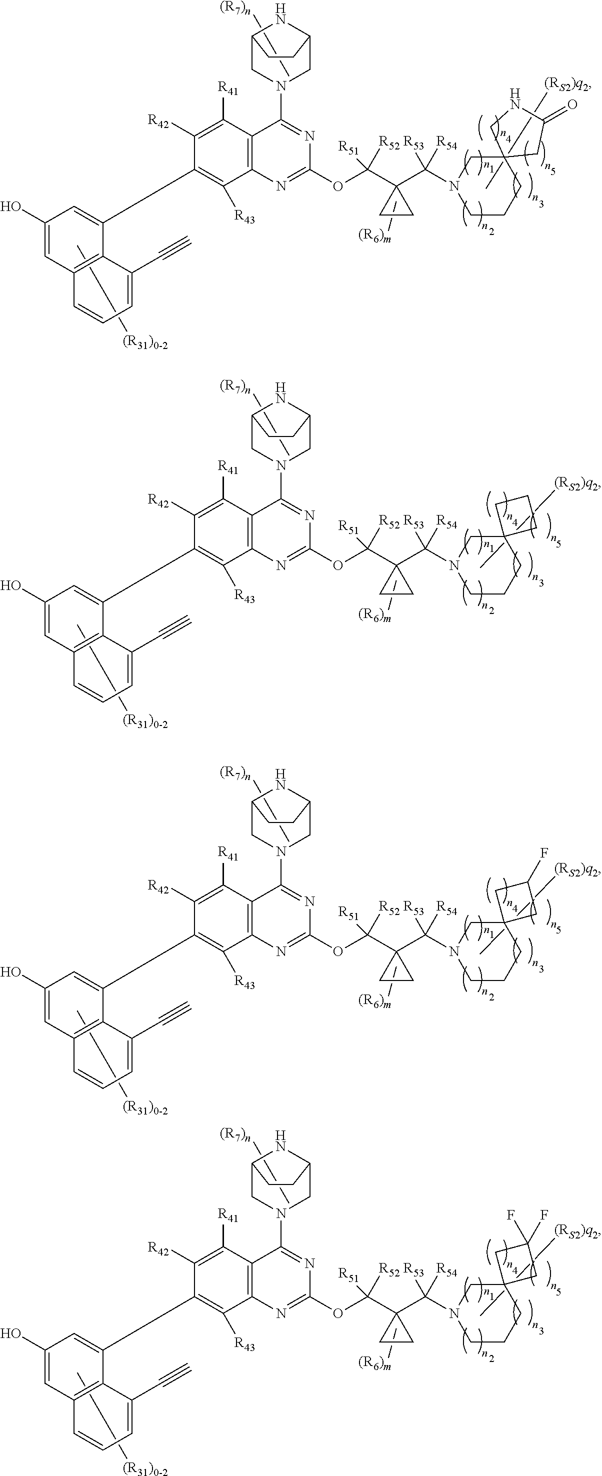

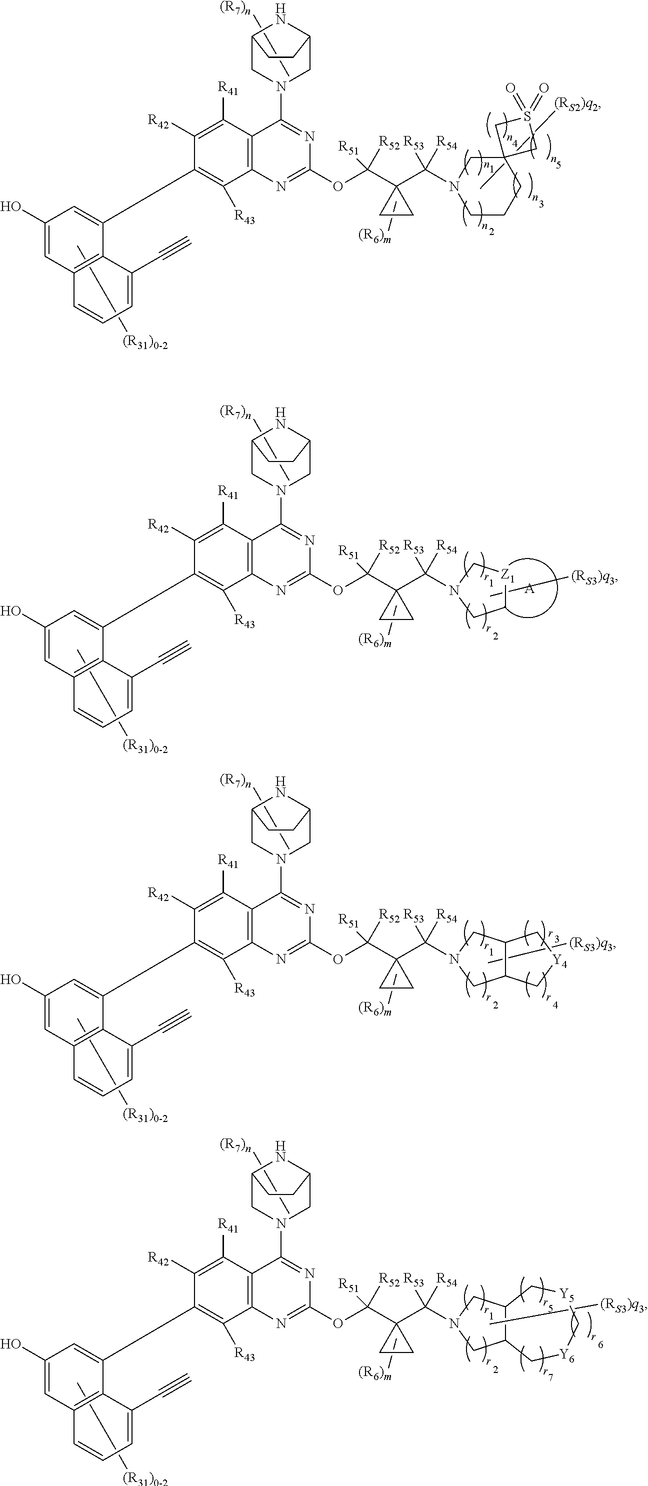

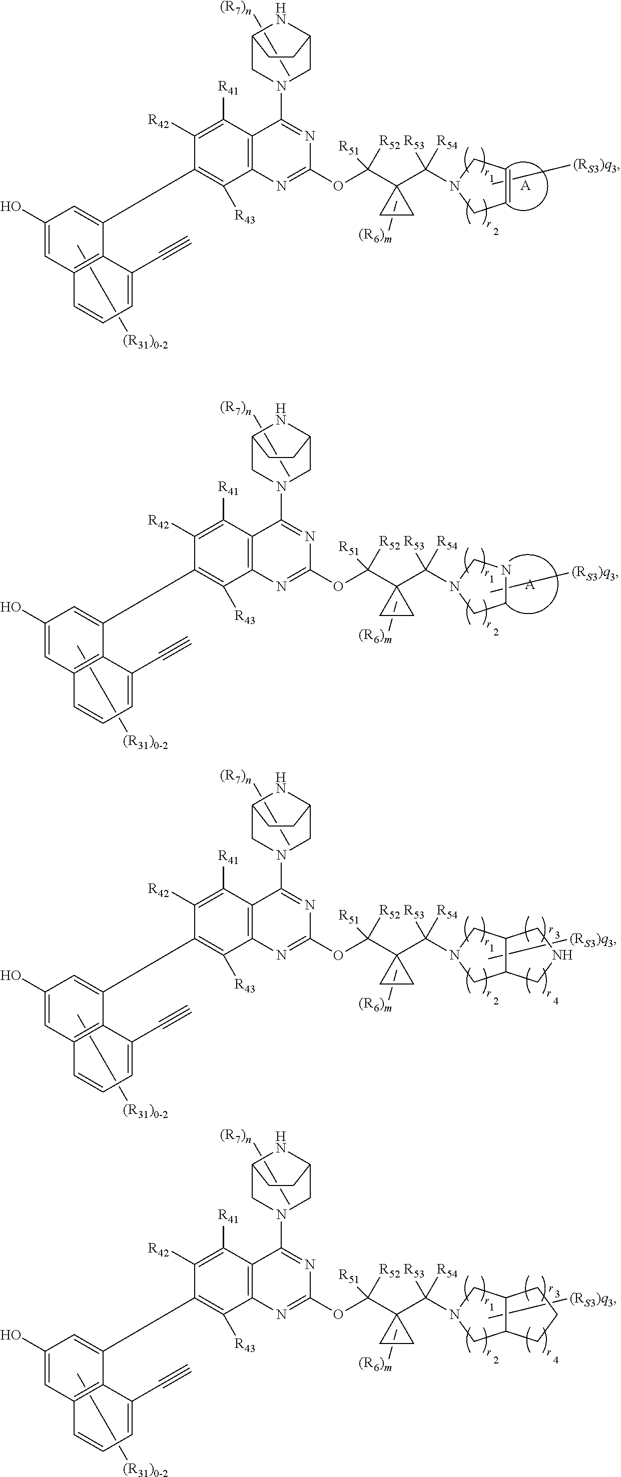

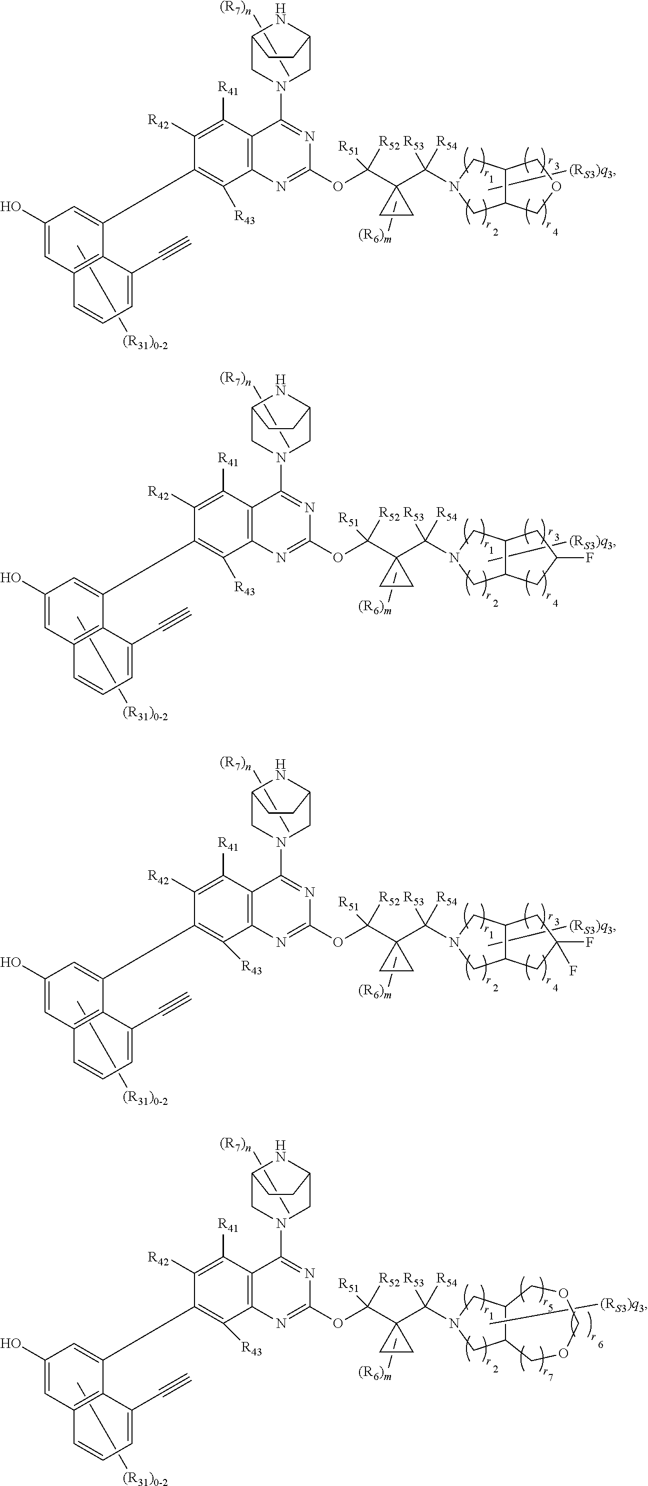

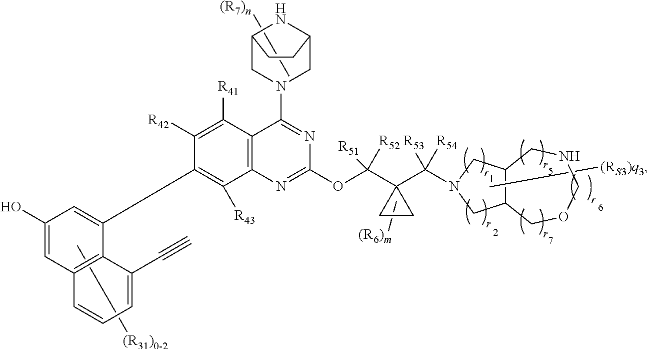

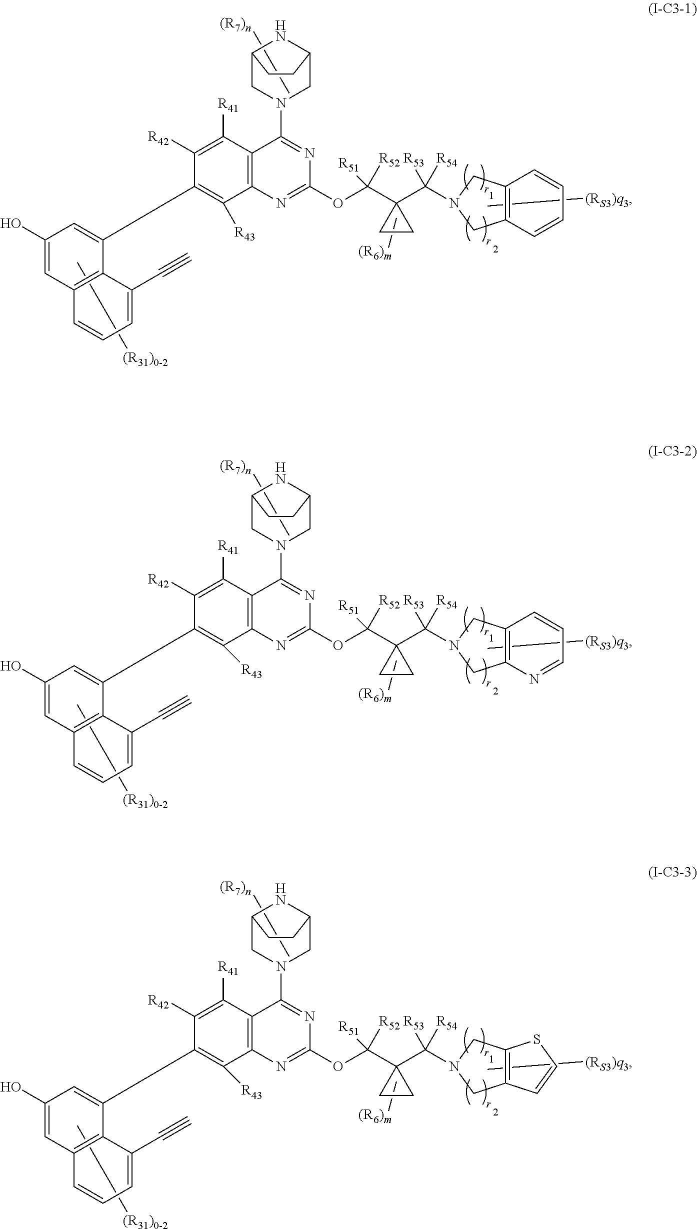

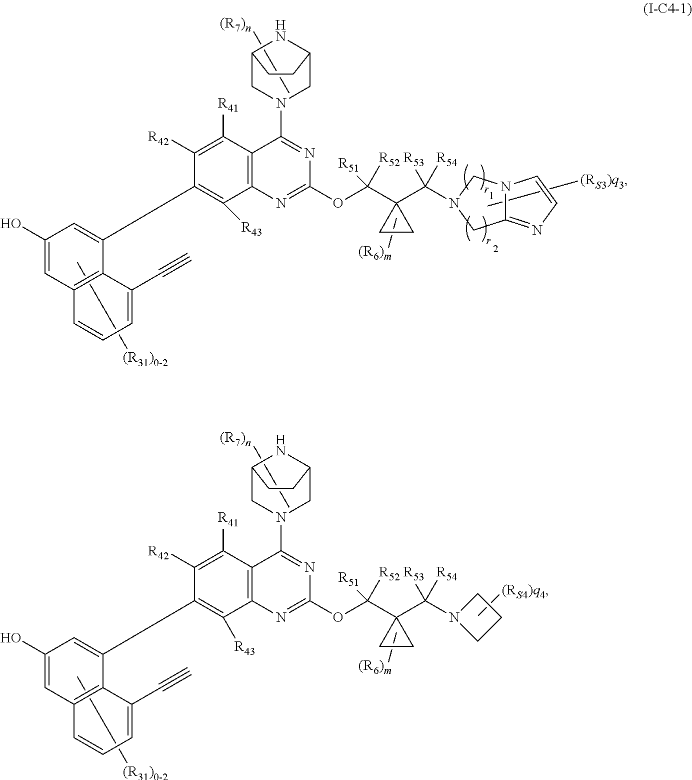

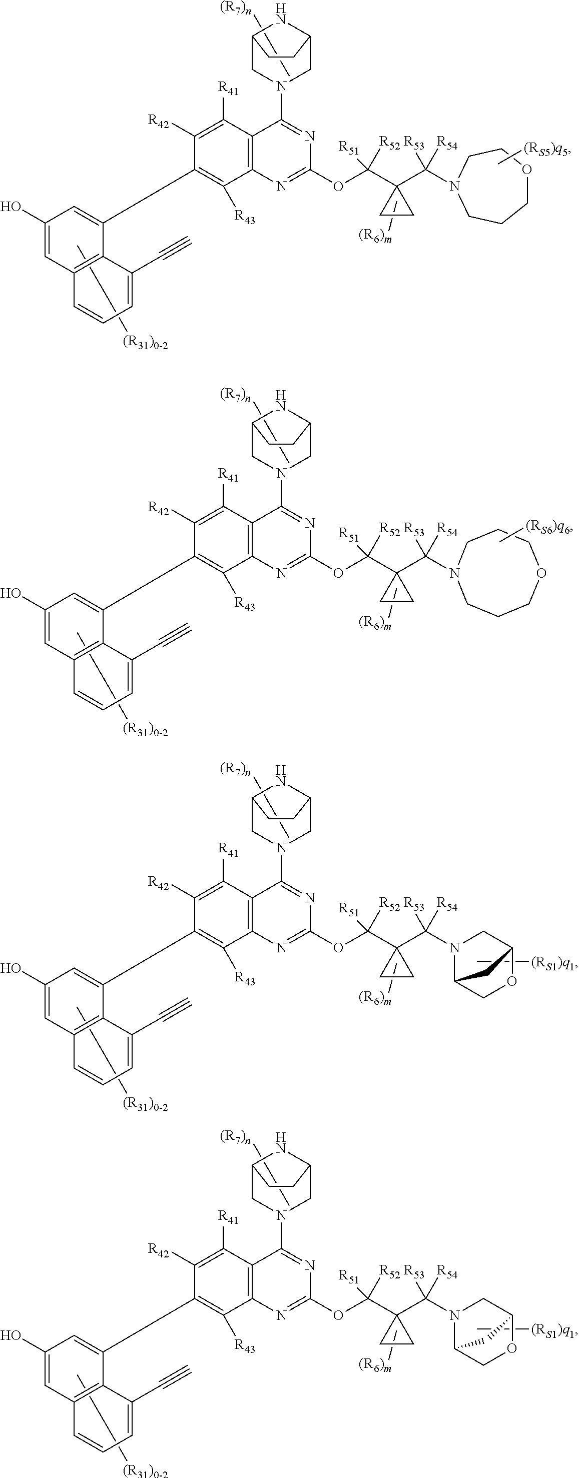

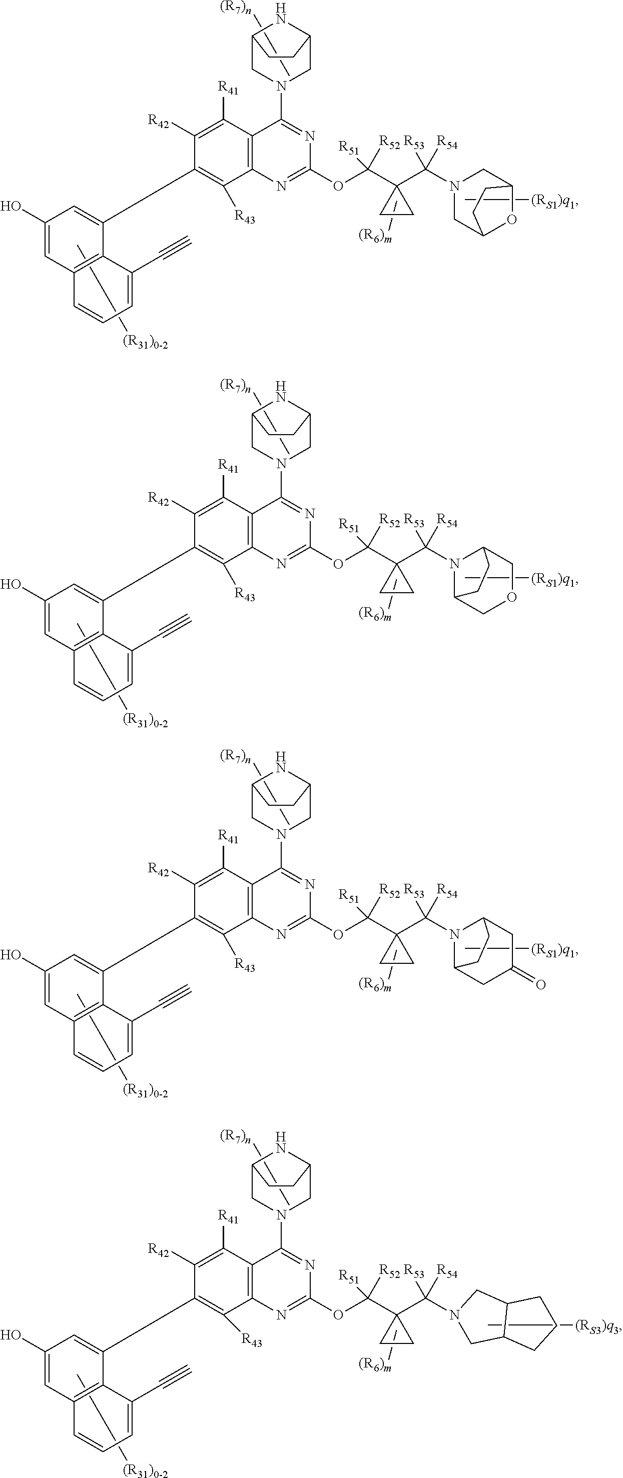

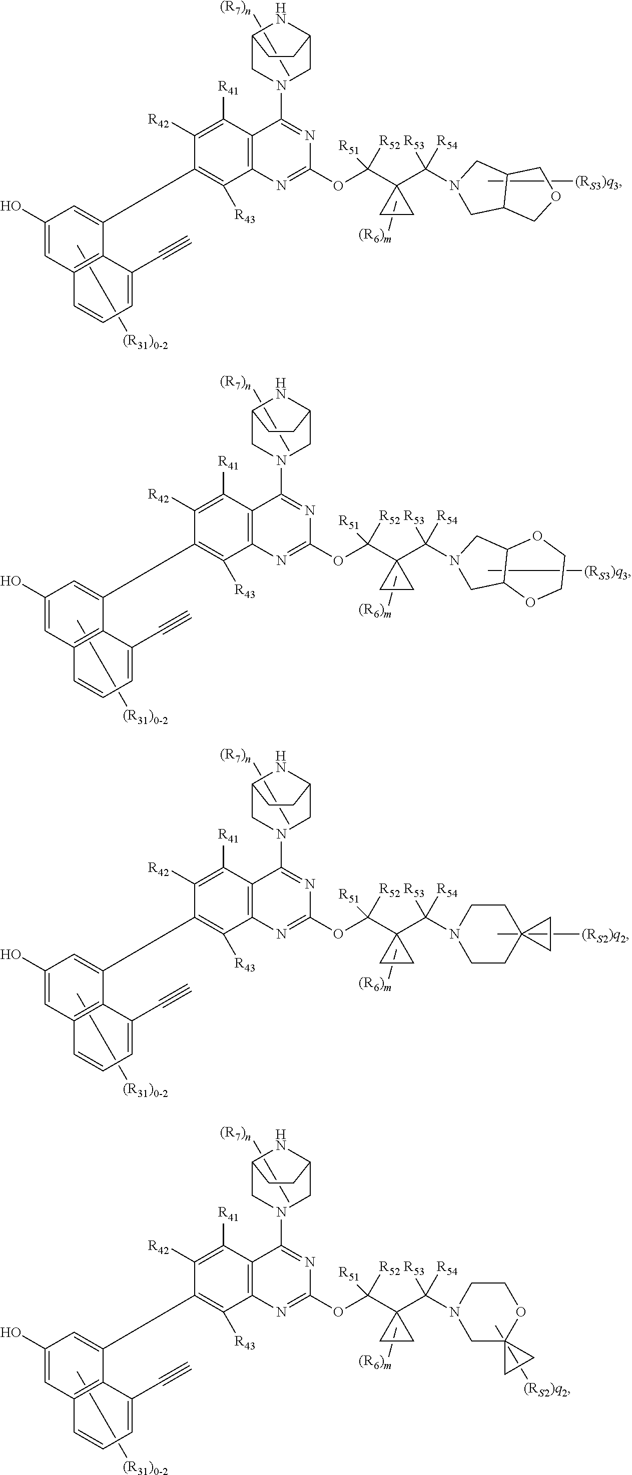

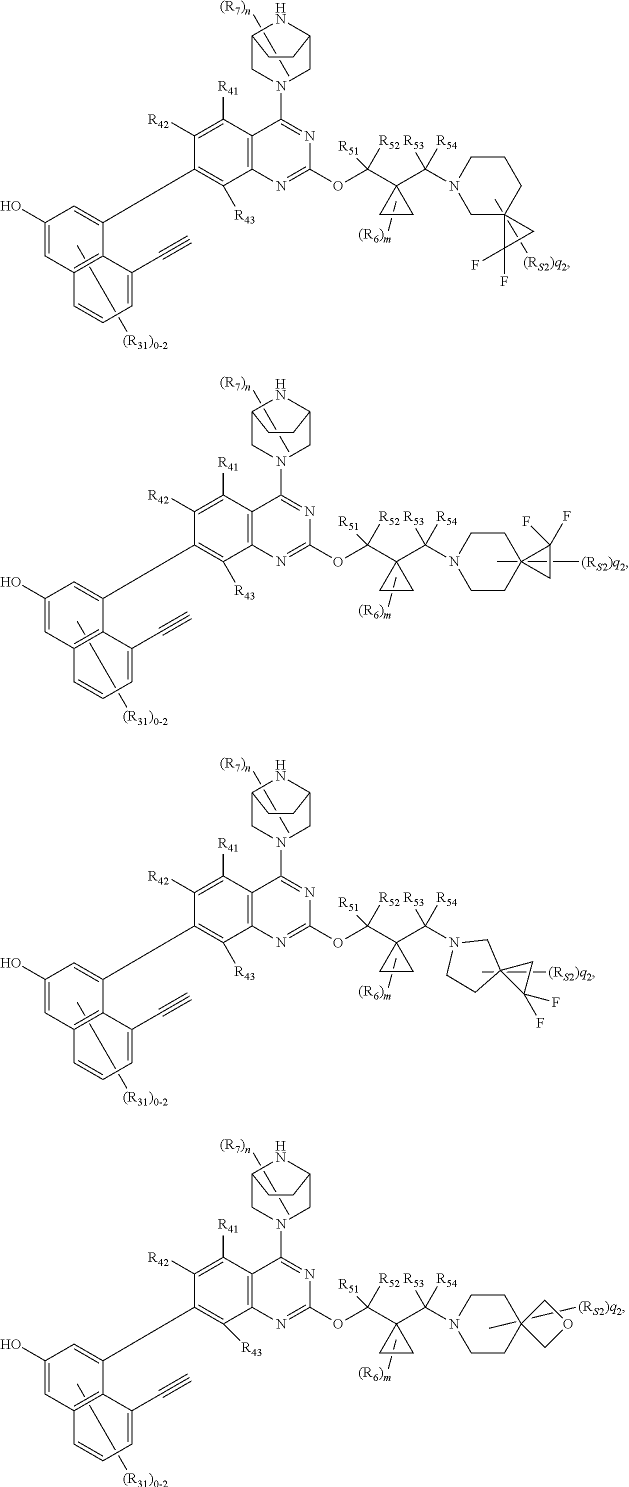

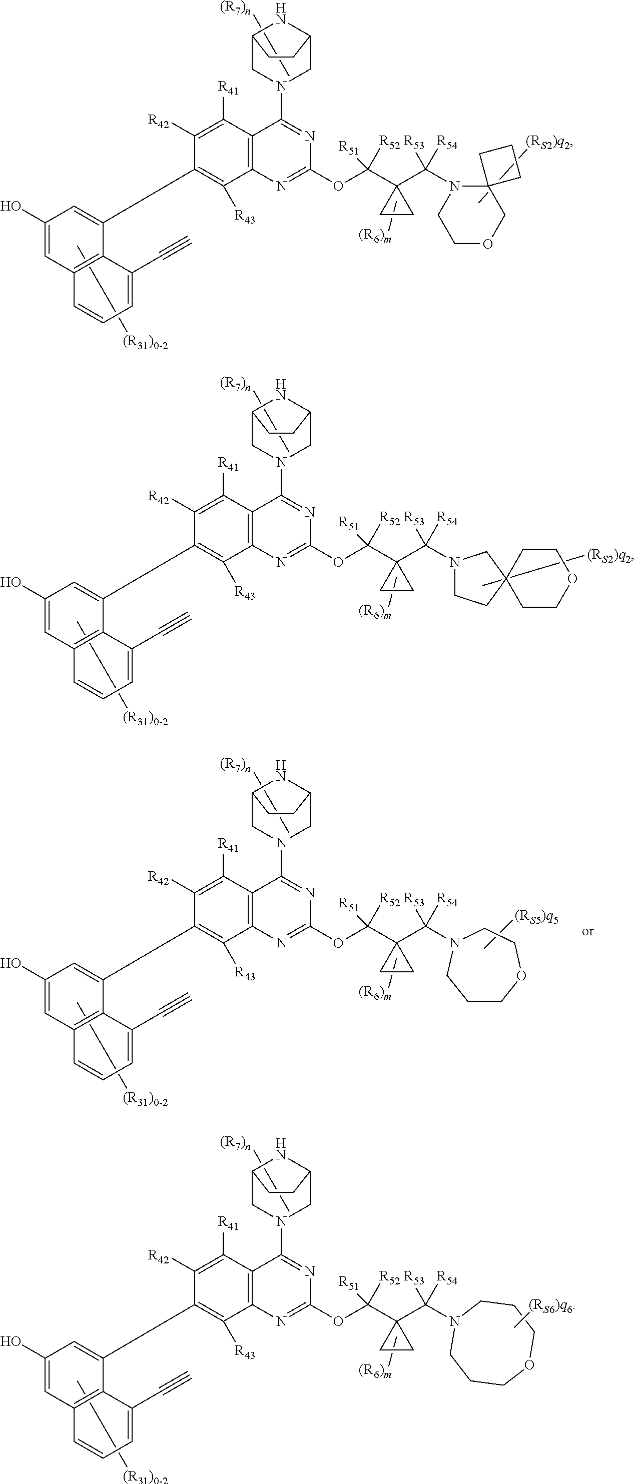

127. The compound according to claim 115, wherein,
R₄₁ at each occurrence is independently selected from —H;
R₄₂ at each occurrence is independently selected from —H, —Cl, —F, —CH₃, —CH₂CH₃, —CH(CH₃)₂, —C(CH₃)₃, 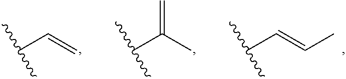
—CF₃, —CN, —NH₂, —NH(CH₃), —N(CH₃)₂, —OH, —O—CH₃, —S(═O)CH₃, —S(═O)₂CH₃, —P(═O)(CH₃)₂, —S—CF₃, —CH₂—OH, —CH₂CH₂—CN, —C(═O)(CH₃), 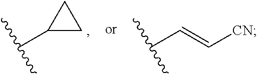
R₄₃ at each occurrence is independently selected from —H, —Cl, —F, —CH₃, —CH₂CH₃, —CH(CH₃)₂, —C(CH₃)₃, 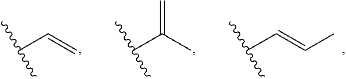
—CF₃, —CN, —NH₂, —NH(CH₃), —N(CH₃)₂, —OH, —O—CH₃, —S(═O)CH₃, —S(═O)₂CH₃, —P(═O)(CH₃)₂, —S—CF₃, —CH₂—OH, —CH₂CH₂—CN, —COOH, —C(═O)(CH₃),

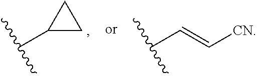

128. The compound according to claim 115, wherein, R₅₁, R₅₂, R₅₃, R₅₄ or R₅₅ at each occurrence is independently selected from —H.

129. The compound according to claim 115, wherein, R₆ at each occurrence is independently selected from —H, —Cl, —F, —Br, —CH₃, —CH₂CH₃, —CH(CH₃)₂, —C(CH₃)₃, —O—CF₃, —S—CF₃, —CF₃, —CN, oxo, —NH₂, —NH(CH₃), —N(CH₃)₂, —OH, —O—CH₃, —O—CH(CH₃)₂, —SH, —S—CH₃, —S—CH(CH₃)₂, —S(═O)CH₃, —S(═O)₂CH₃, —COOH, —C(═O)(CH₃), —C(═O)(CH₂CH₃), —C(═O)(CF₃), —C(═O)NH₂, —C(═O)NH(CH₃), —NHC(═O)(CH₃), —S(═O)NH₂, —S(═O)NH(CH₃), —NHS(═O)(CH₃), —S(═O)₂NH₂, —S(═O)₂NH(CH₃), —NHS(═O)₂(CH₃), —P(═O)H(CH₃), —P(═O)(CH₃)₂, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, or 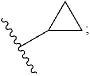 [SMILES: *C1CC1 ; ";"]
m is selected from 0, 1, 2, 3, 4, 5, or 6; preferably, m is selected from 0, 1, 2, or 3, more preferably, m is selected from 0.

130. The compound according to claim 115, wherein, R₇ at each occurrence is independently selected from —H, —Cl, —F, —Br, —CH₃, —CH₂CH₃, —CH(CH₃)₂, —C(CH₃)₃, —O—CF₃, —S—CF₃, —CF₃, —CN, oxo, —NH₂, —NH(CH₃), —N(CH₃)₂, —OH, —O—CH₃, —O—CH(CH₃)₂, —SH, —S—CH₃, —S—CH(CH₃)₂, —S(═O)CH₃, —S(═O)₂CH₃, —COOH, —C(═O)(CH₃), —C(═O)(CH₂CH₃), —C(═O)(CF₃), —C(═O)NH₂, —C(═O)NH(CH₃), —NHC(═O)(CH₃), —S(═O)NH₂, —S(═O)NH(CH₃), —NHS(═O)(CH₃), —S(═O)₂NH₂, —S(═O)₂NH(CH₃), —NHS(═O)₂(CH₃), —P(═O)H(CH₃), —P(═O)(CH₃)₂, —CH₂—OH, —CH₂CH₂—OH, —CH(CH₃)—OH, —CH₂—NH₂, —CH₂CH₂—NH₂, —CH(CH₃)—NH₂, —CH₂—CN, —CH₂CH₂—CN, —CH(CH₃)—CN, or 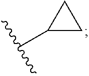 [SMILES: *C1CC1]
n is selected from 0, 1, 2, 3, 4, 5, or 6; preferably, n is selected from 0, 1, 2, or 3, more preferably, n is selected from 0.

131. The compound according to claim 115, wherein, the compound is selected from:

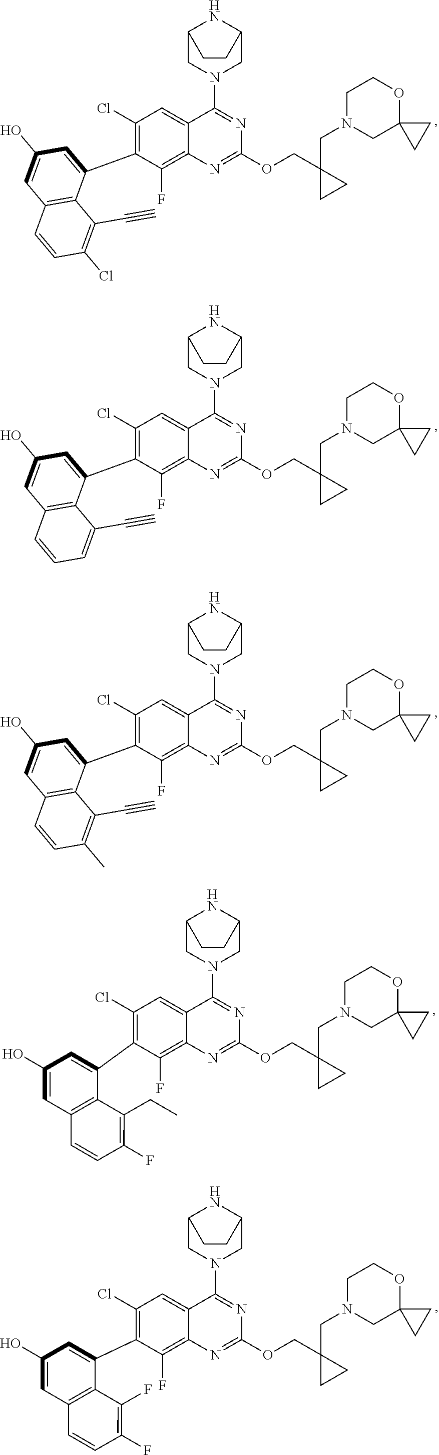

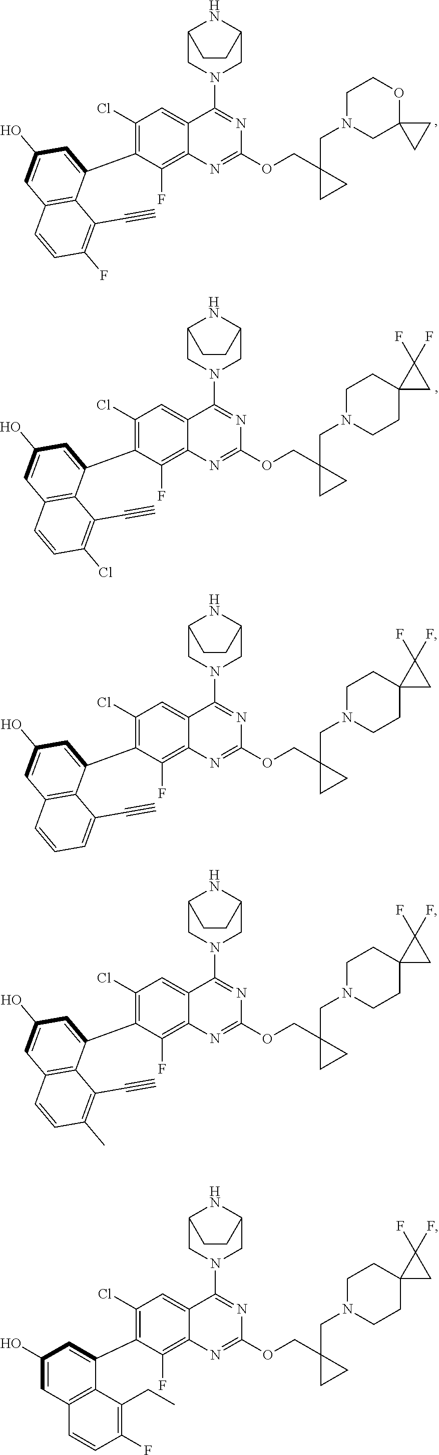

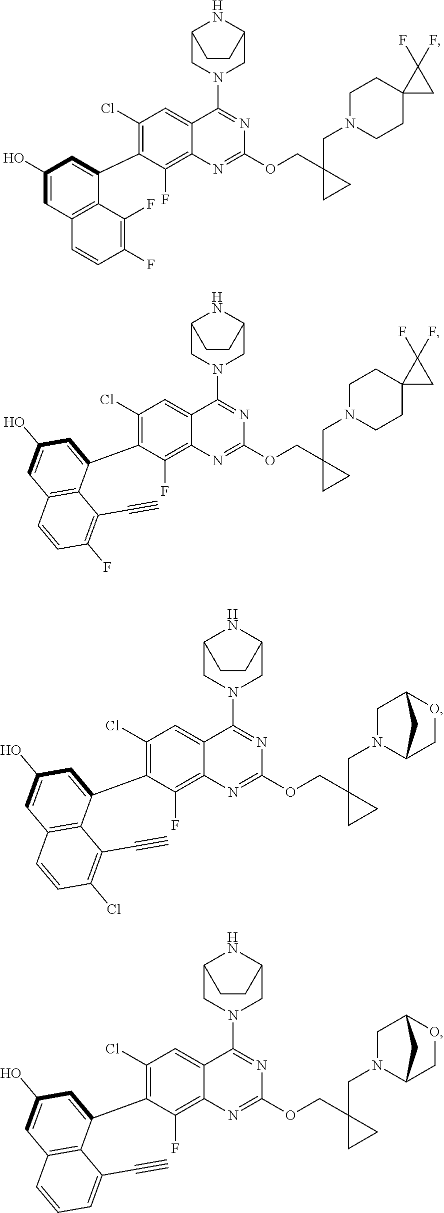

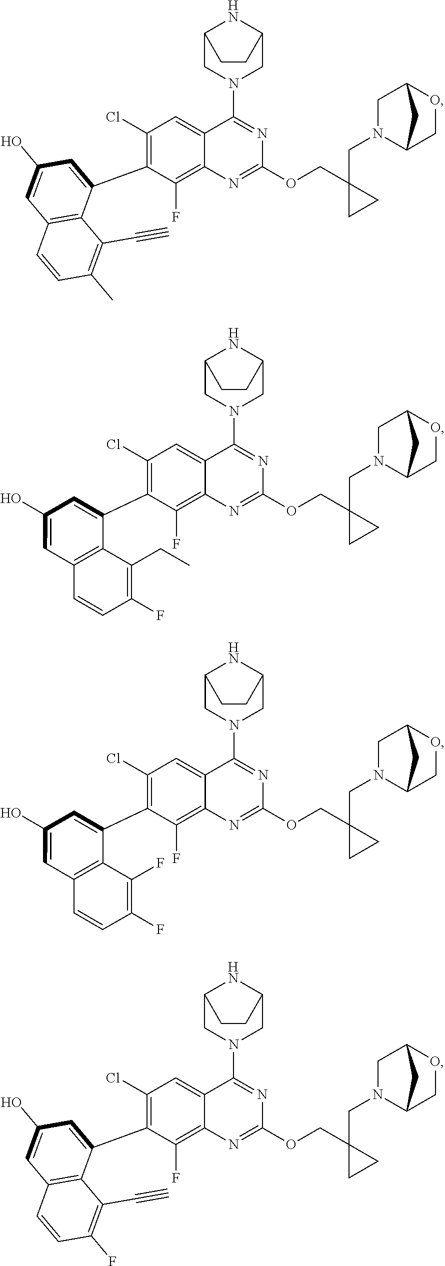

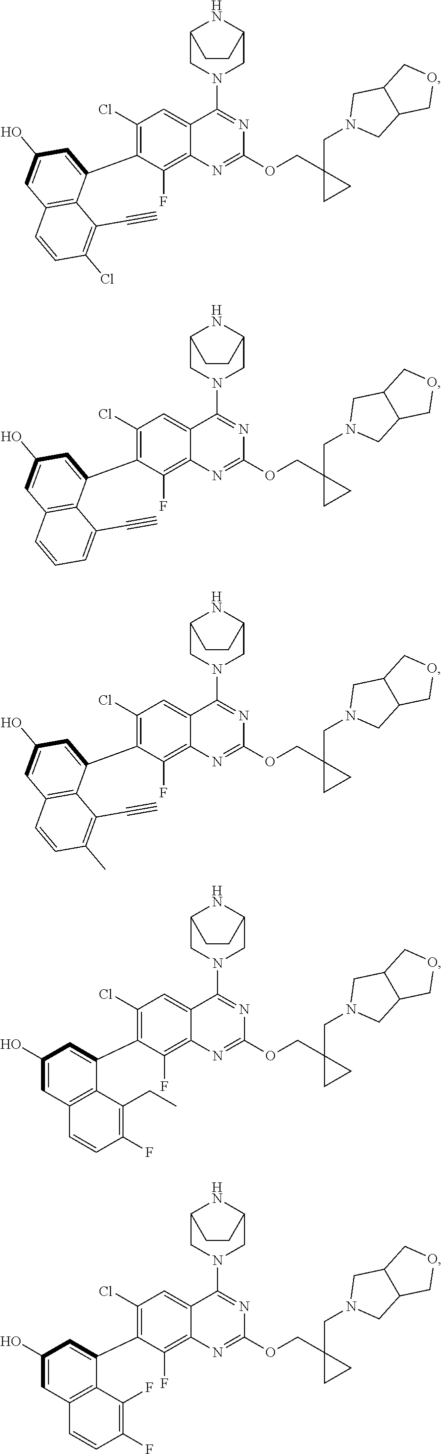

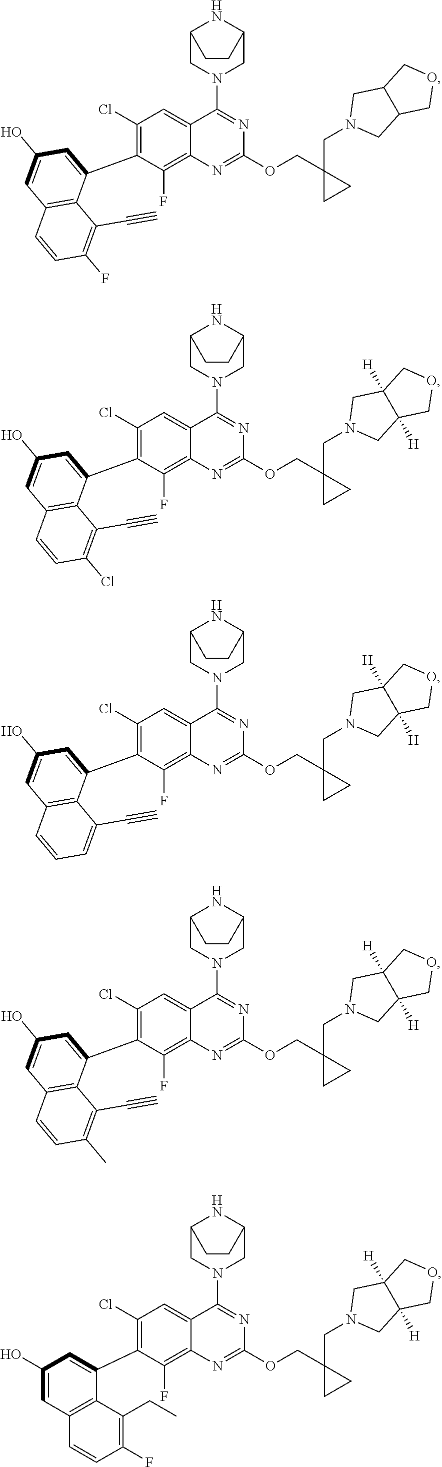

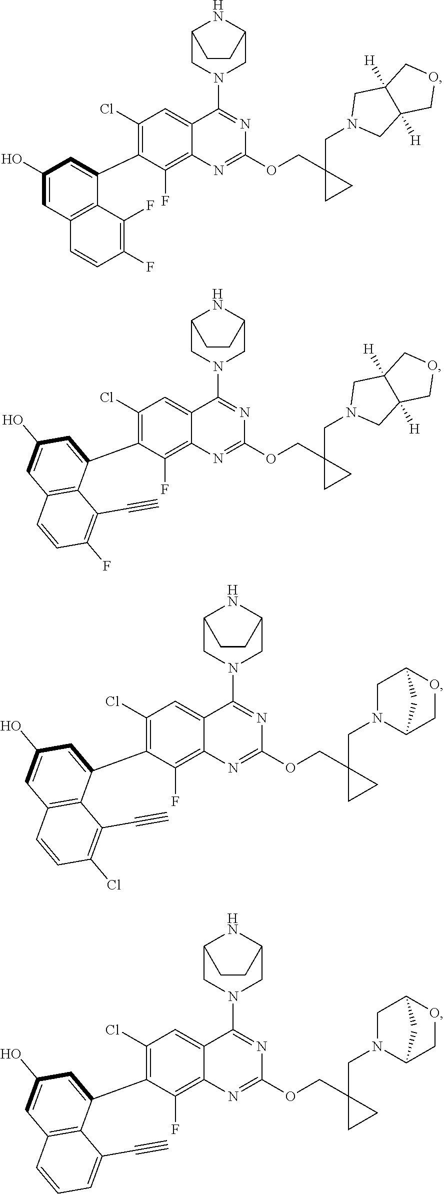

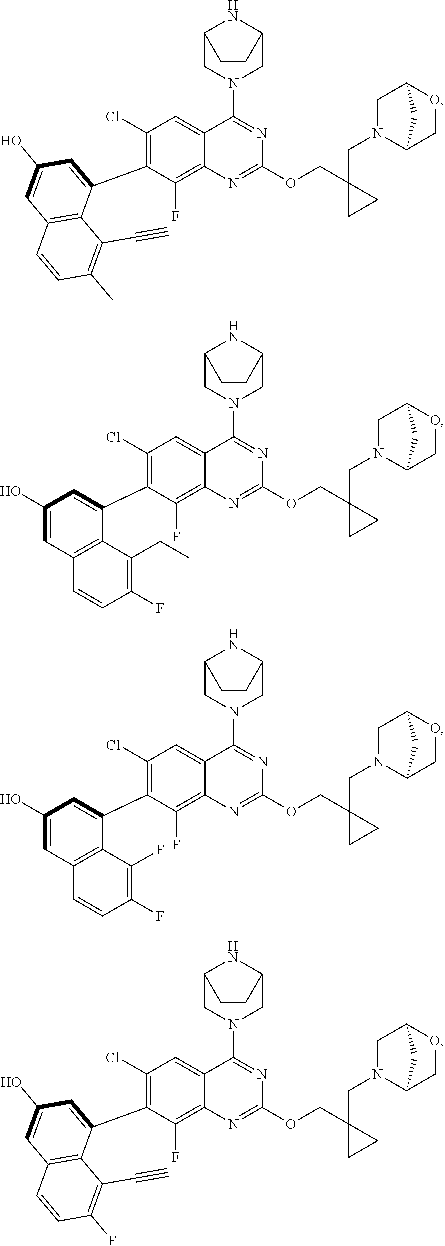

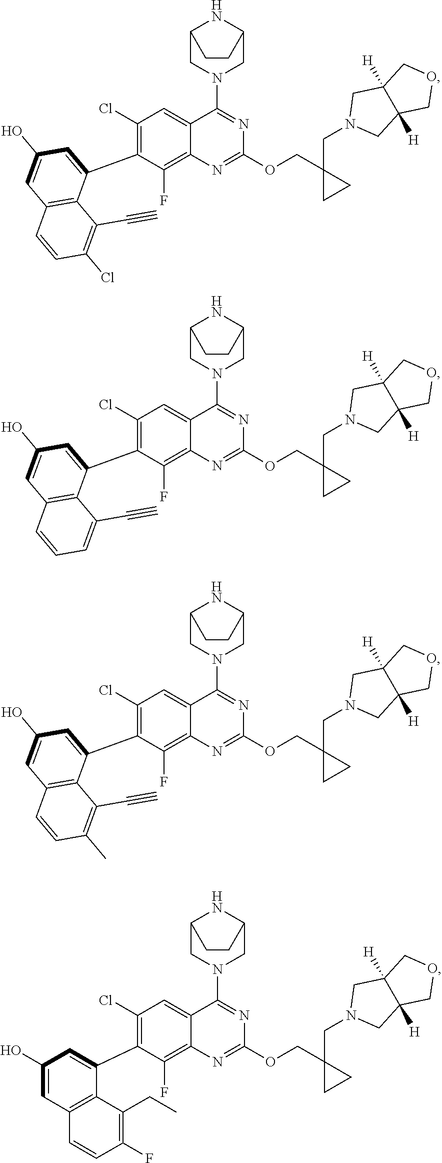

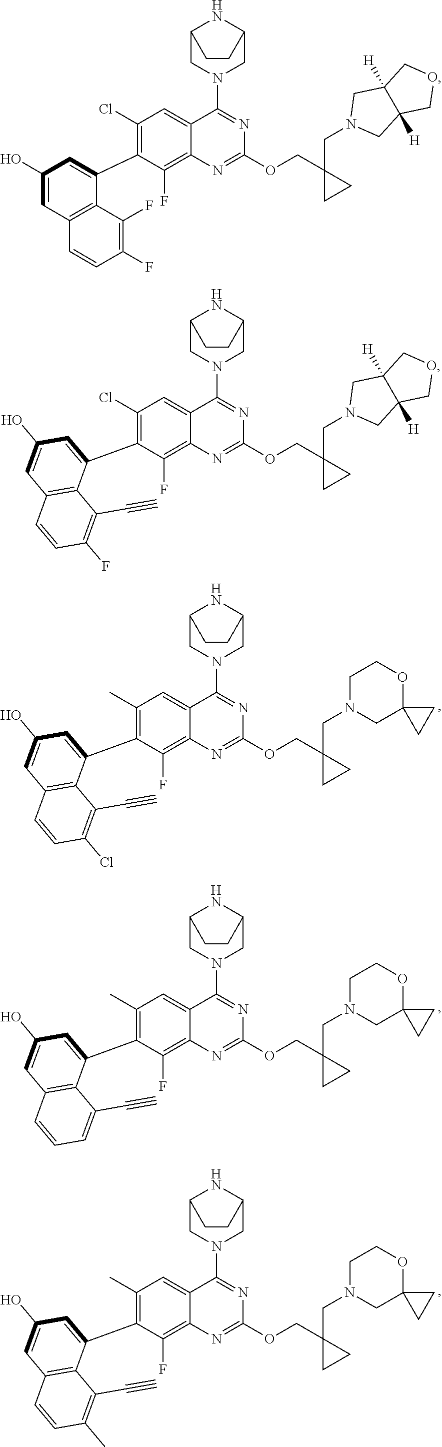

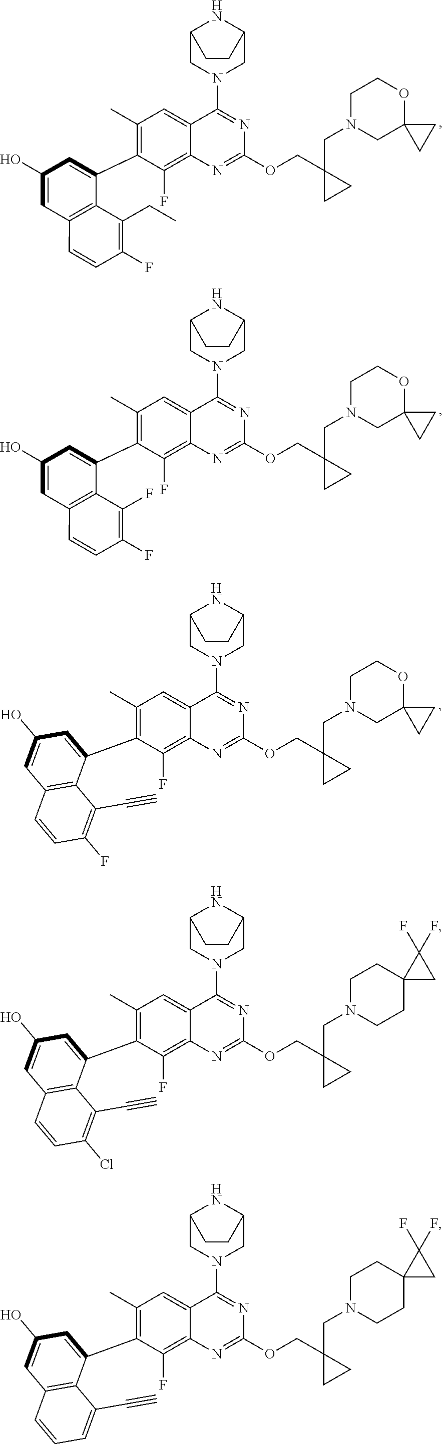

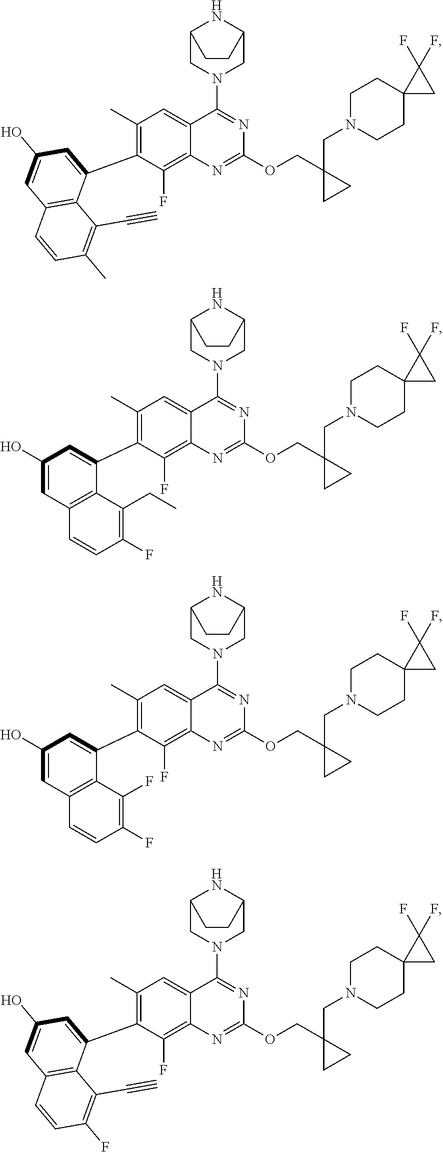

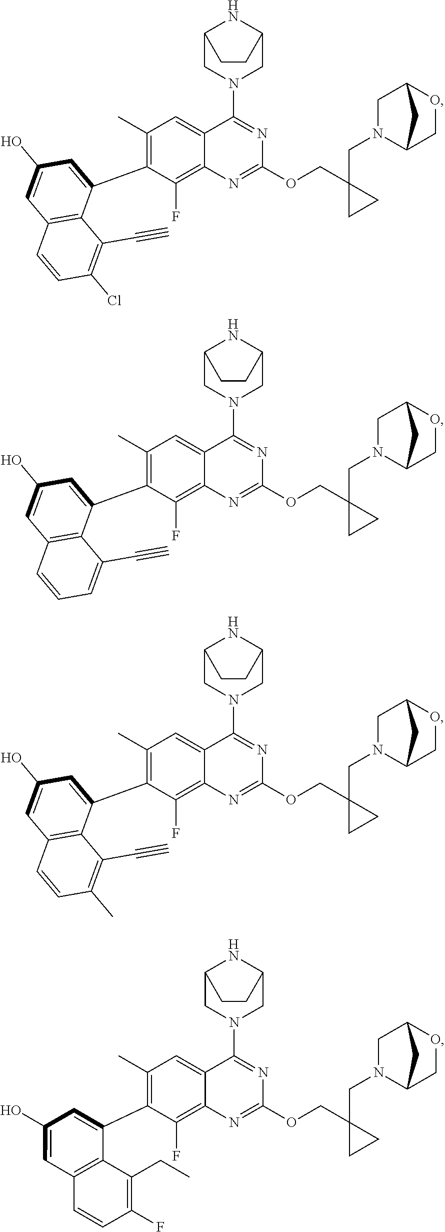

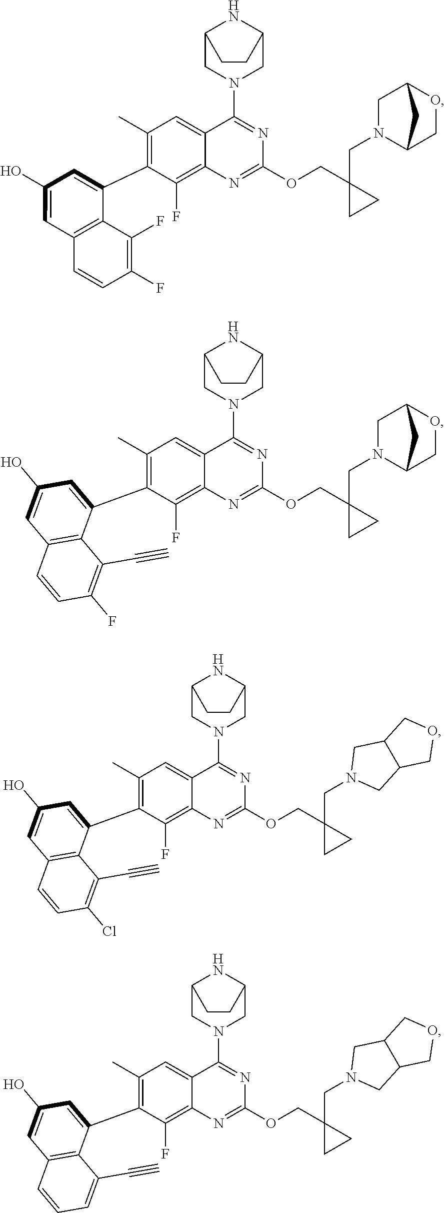

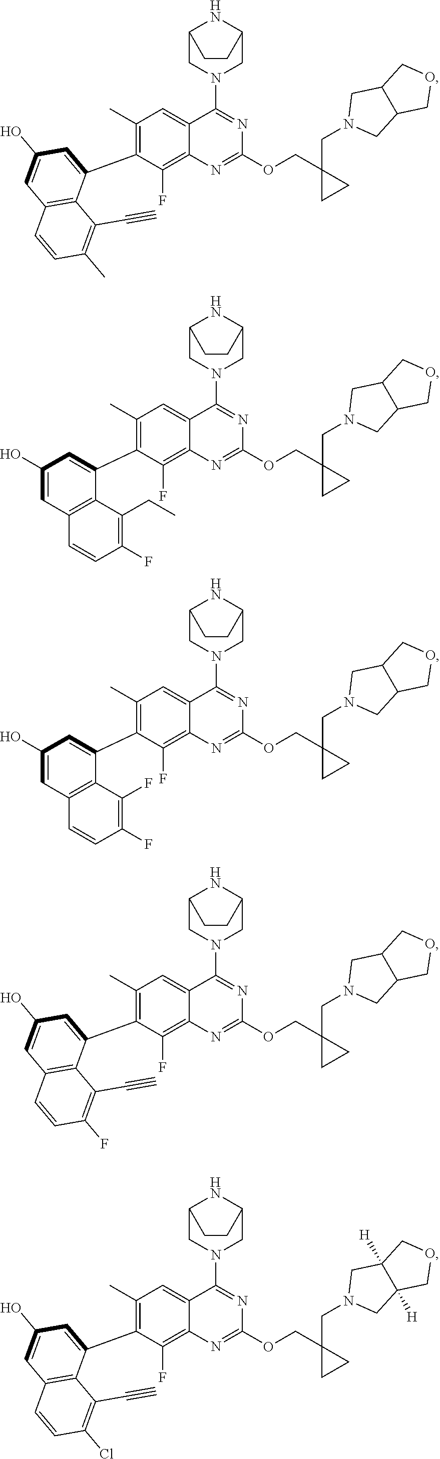

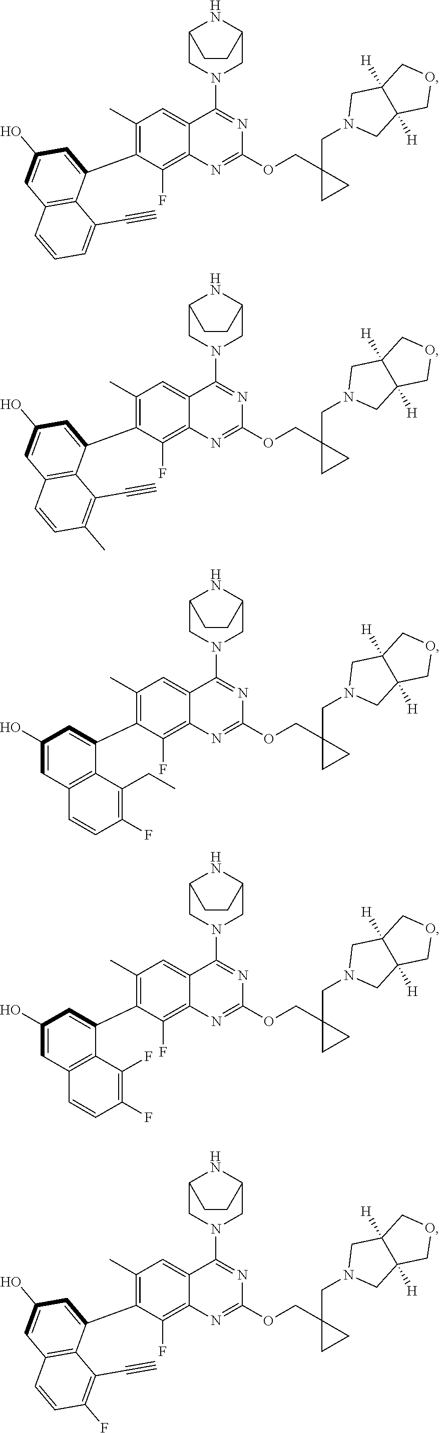

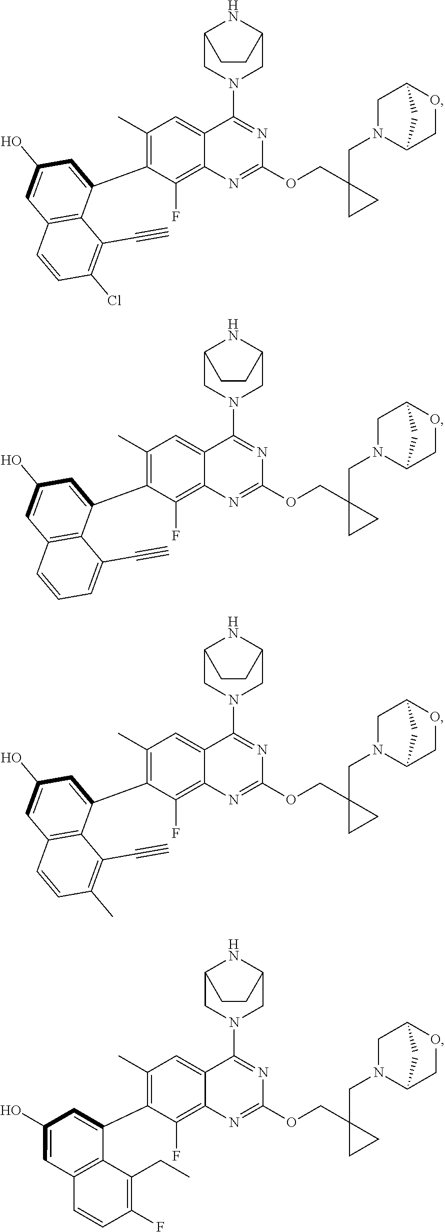

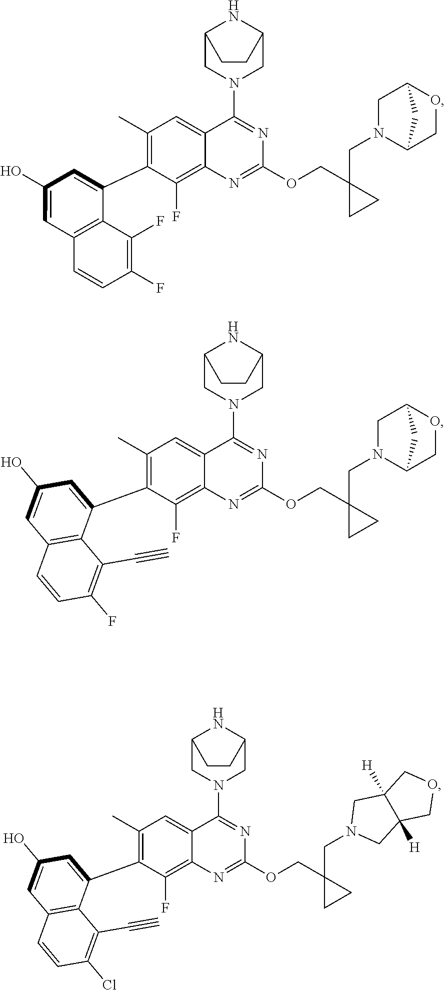

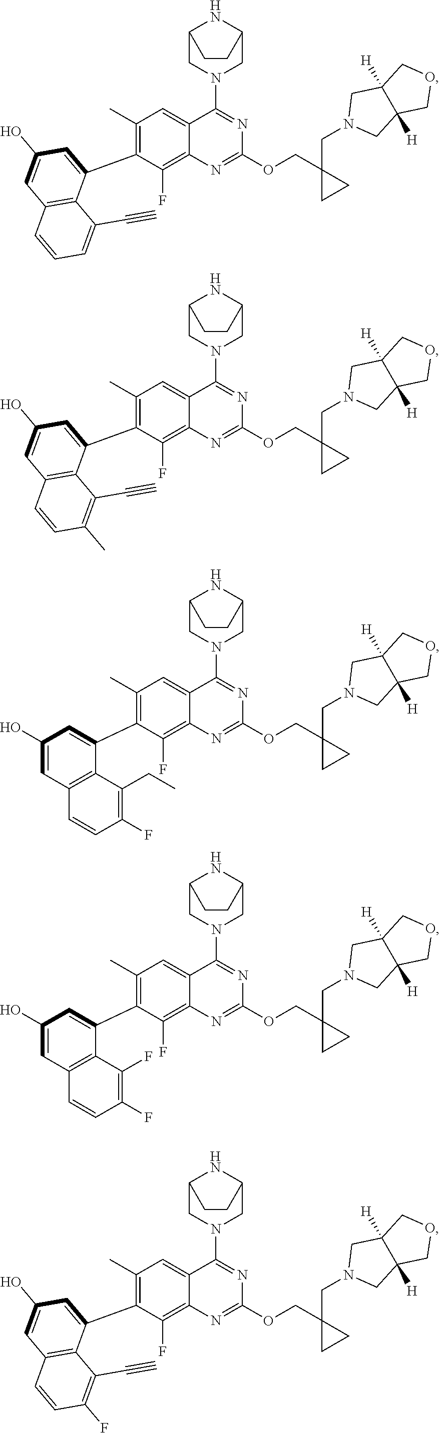

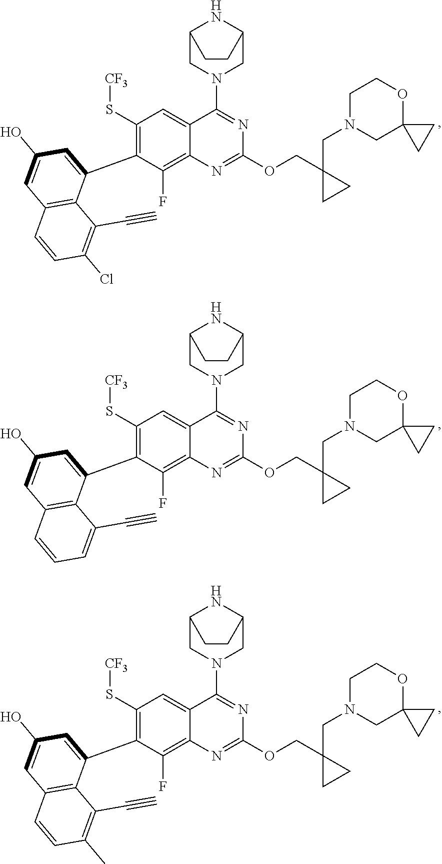

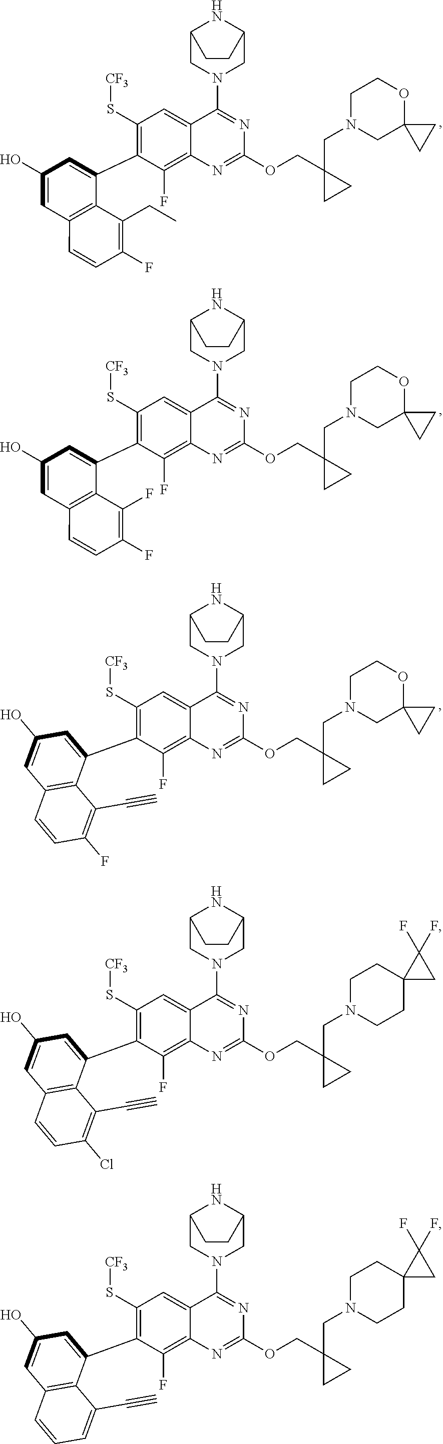

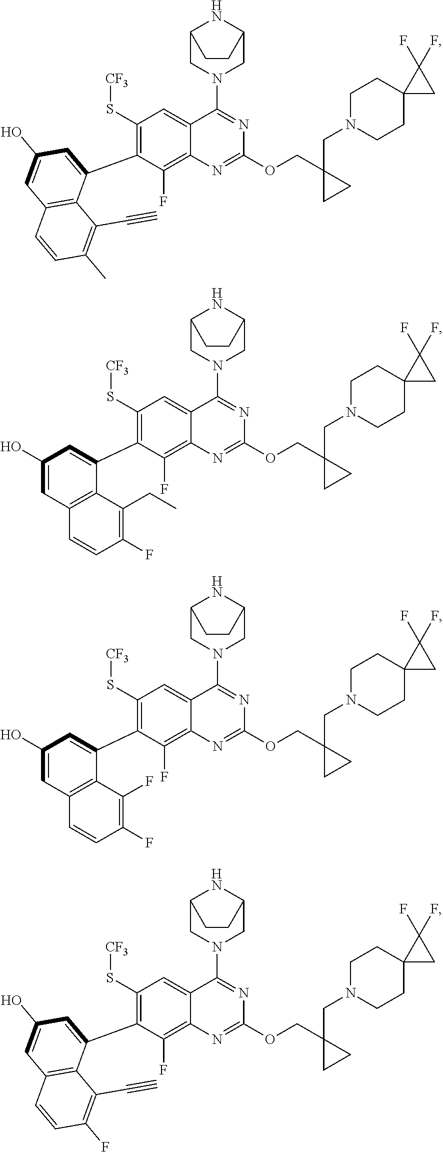

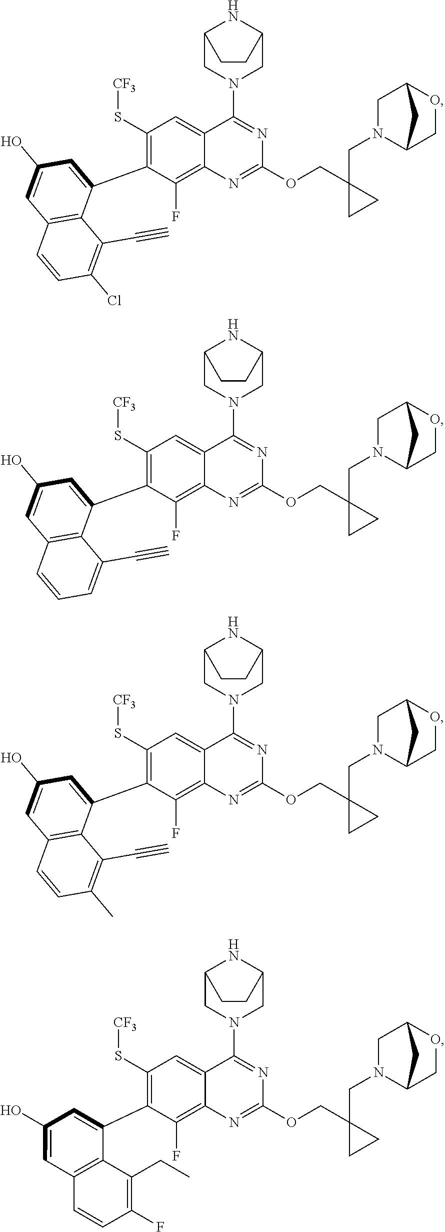

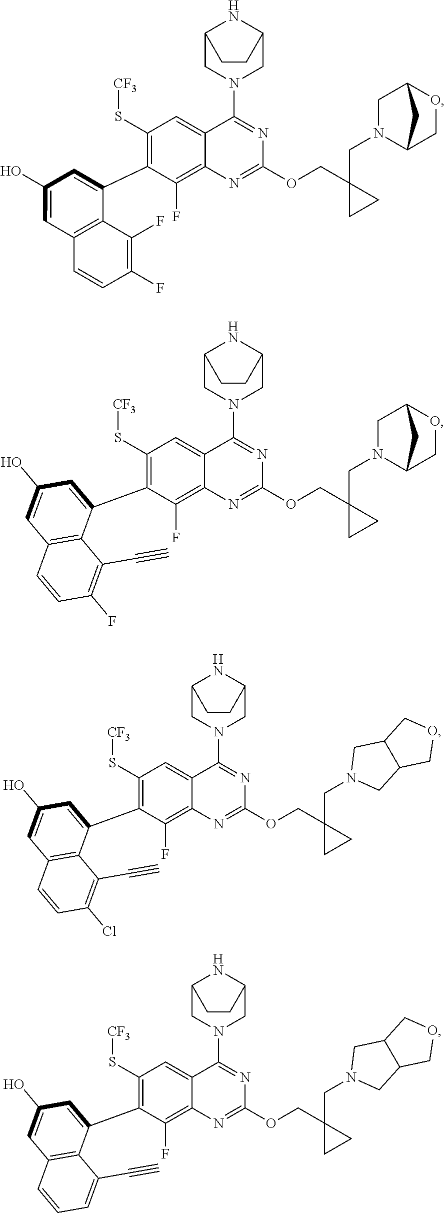

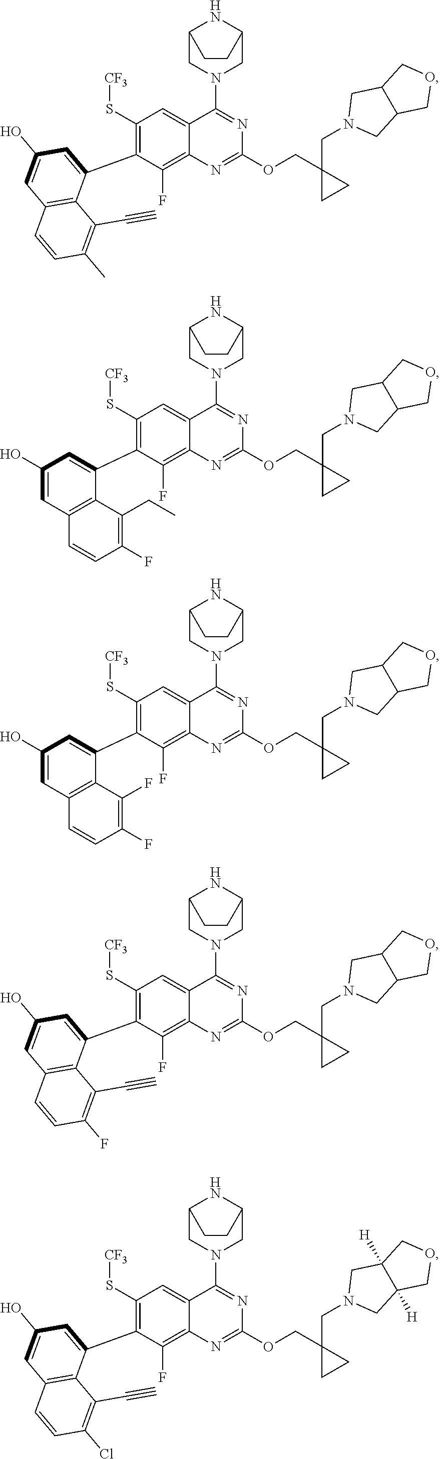

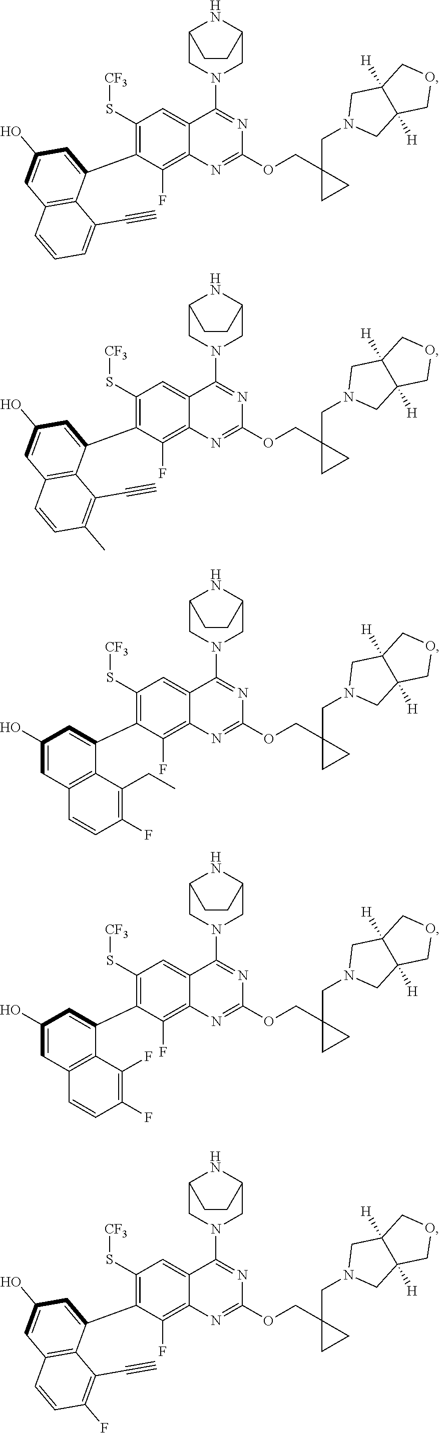

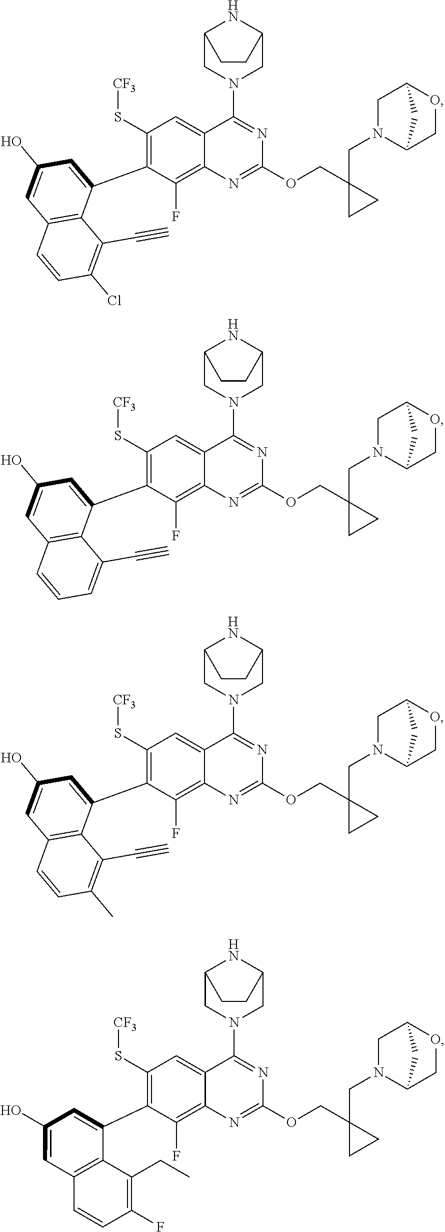

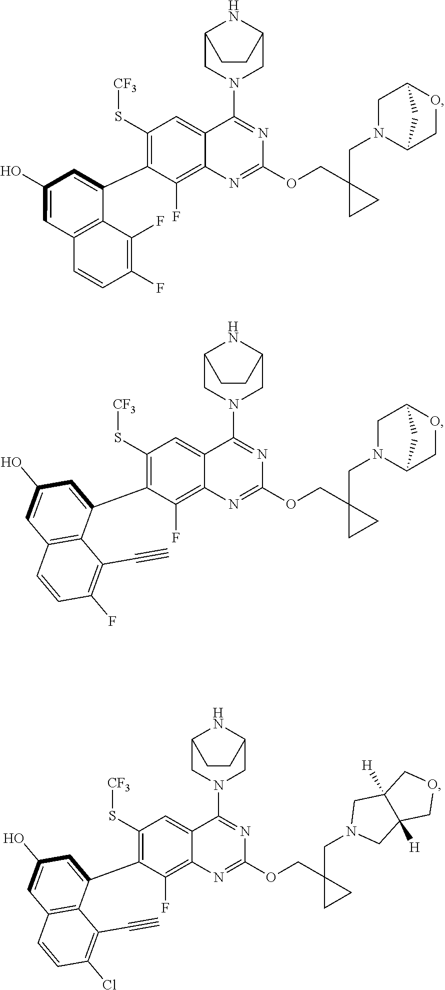

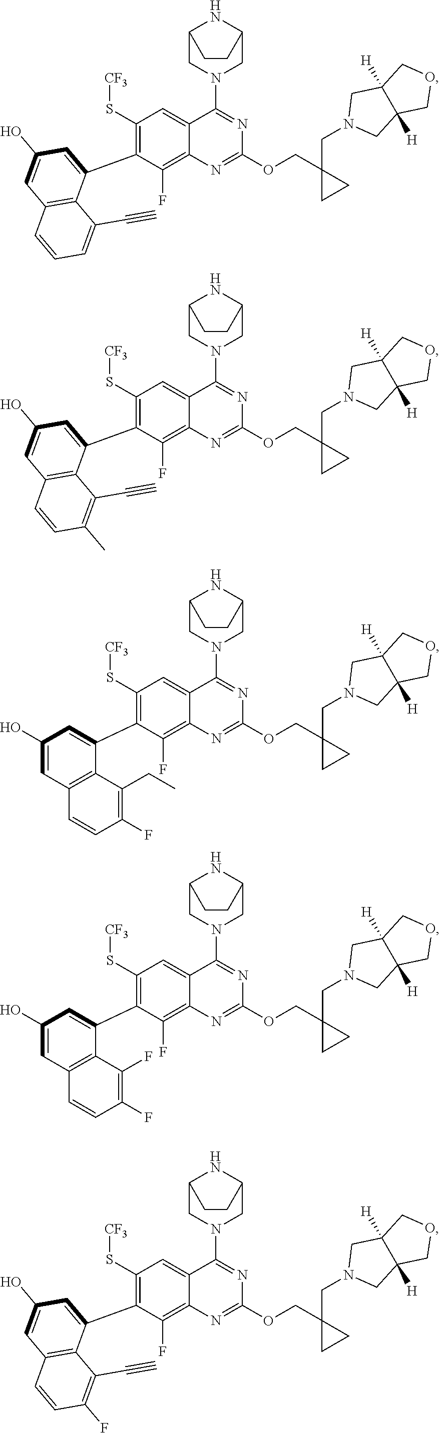

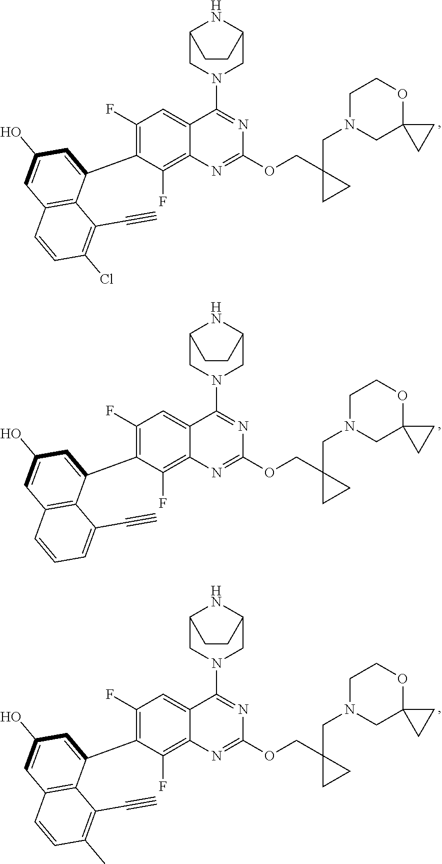

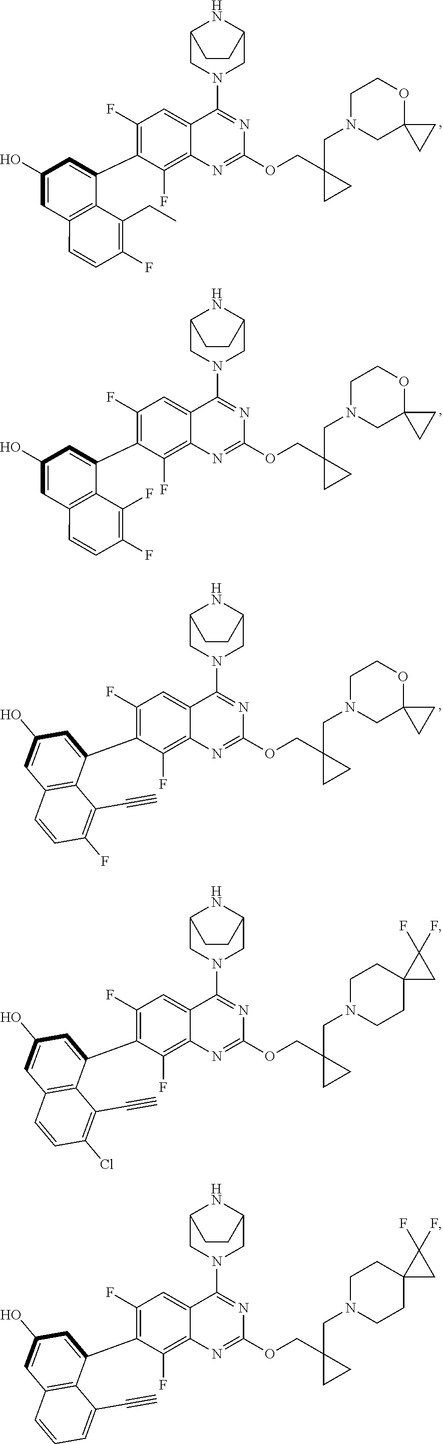

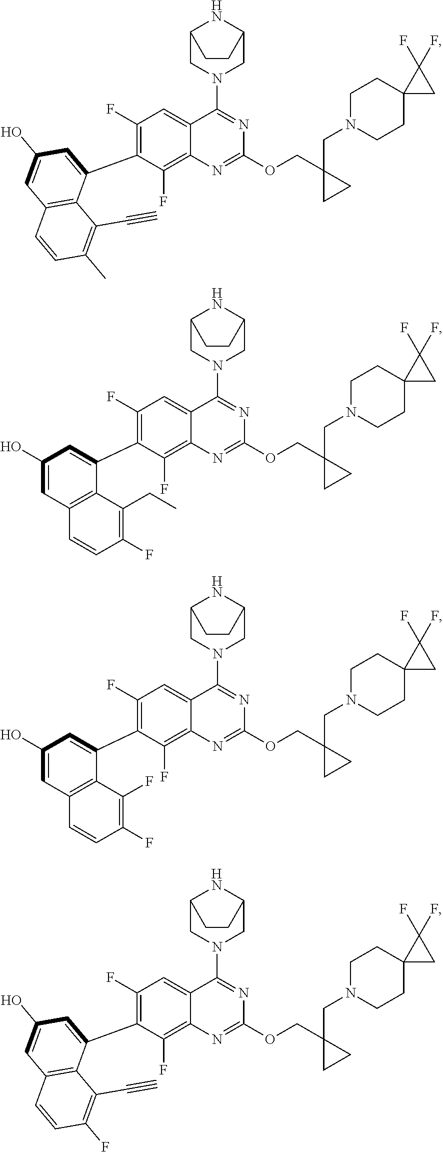

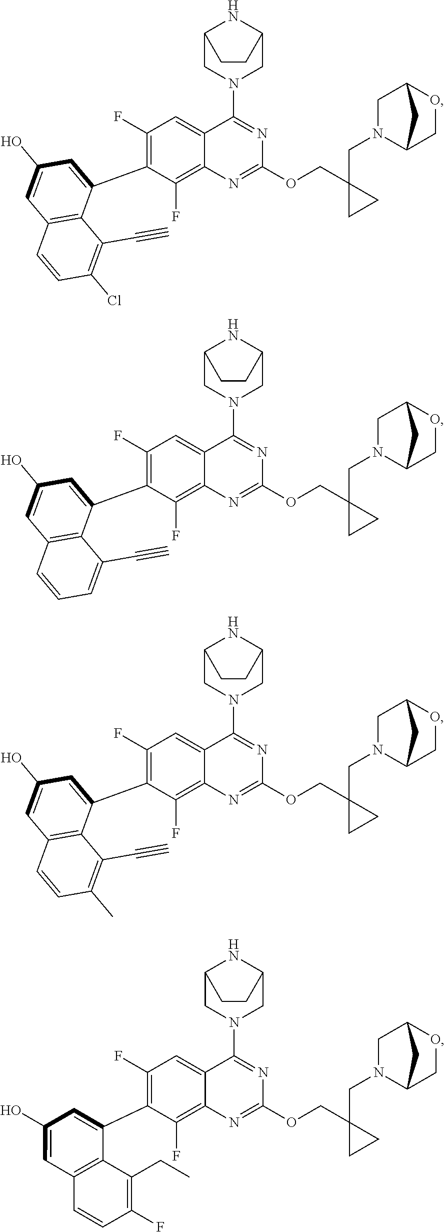

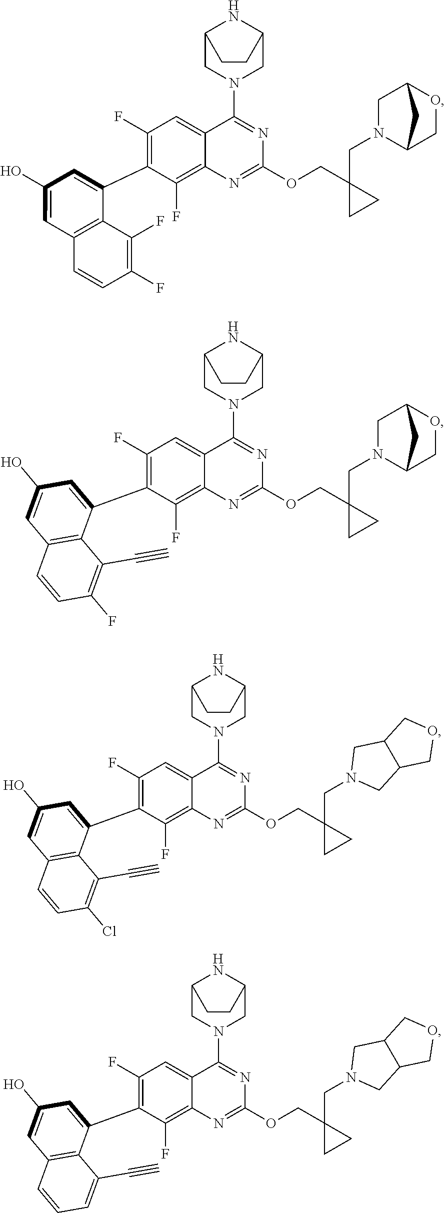

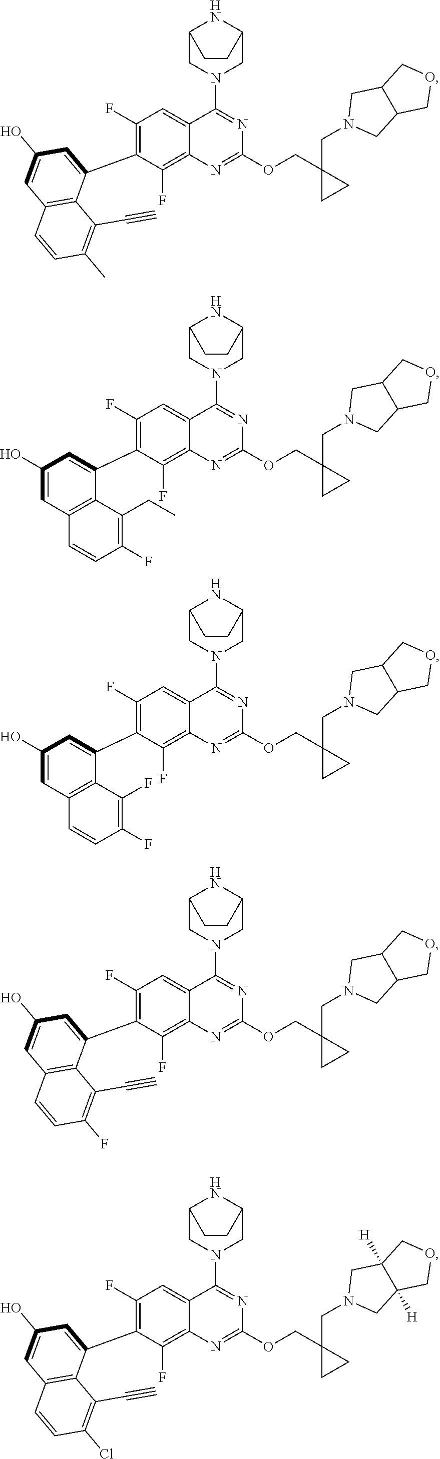

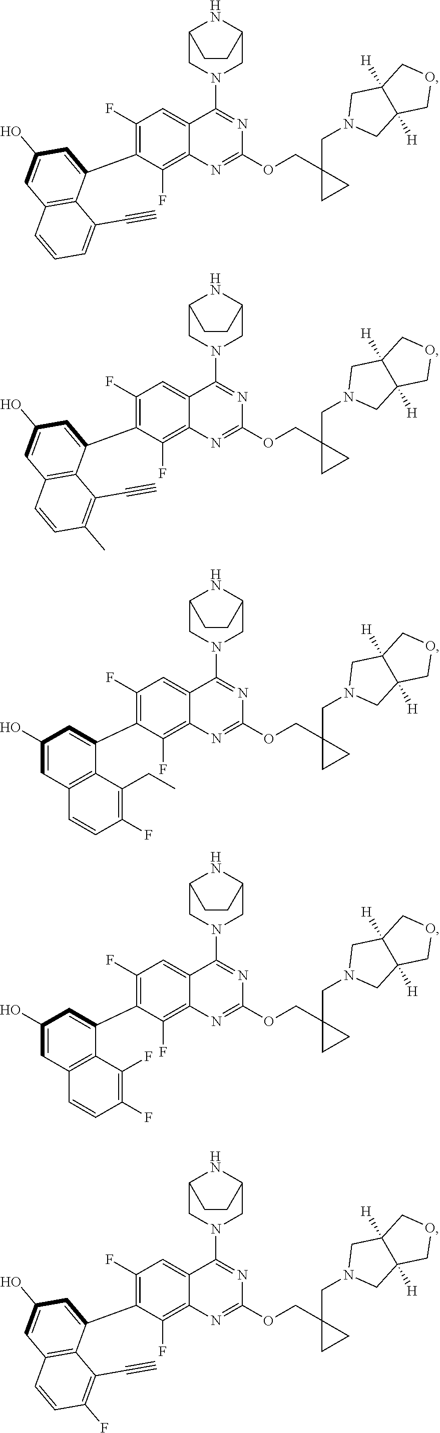

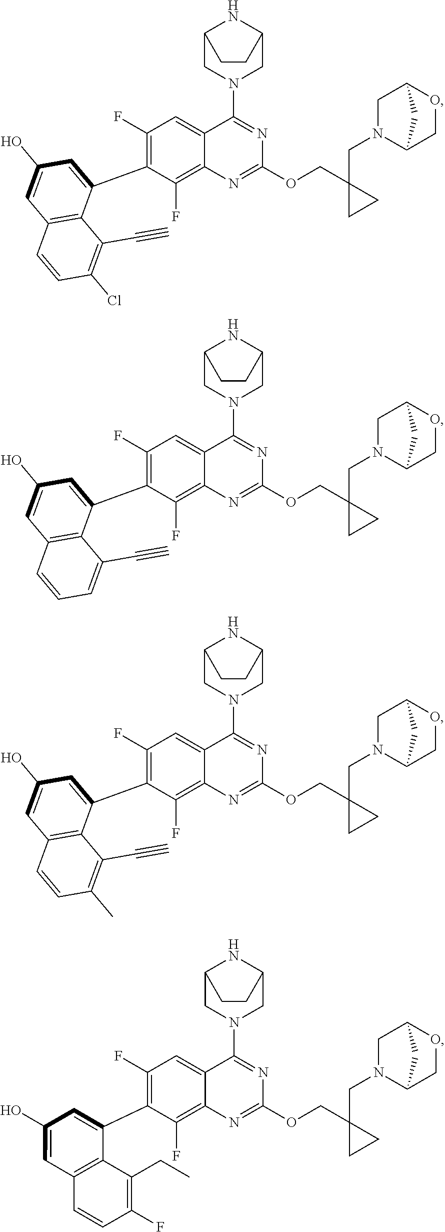

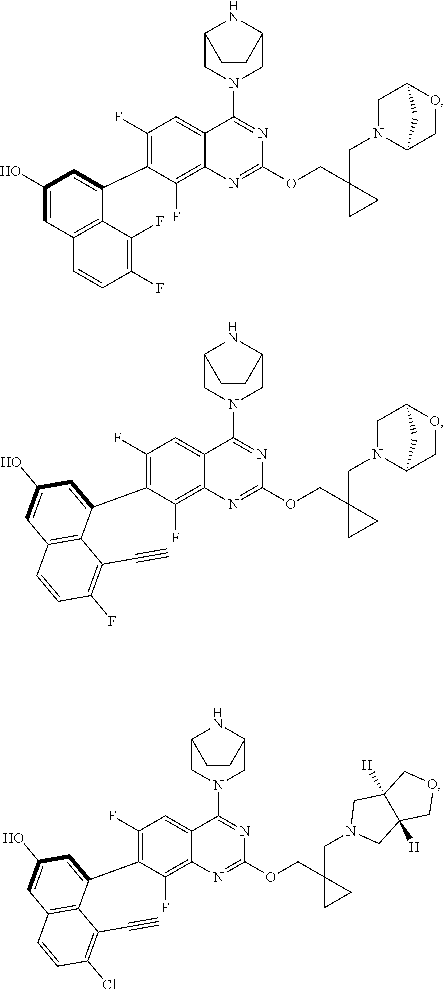

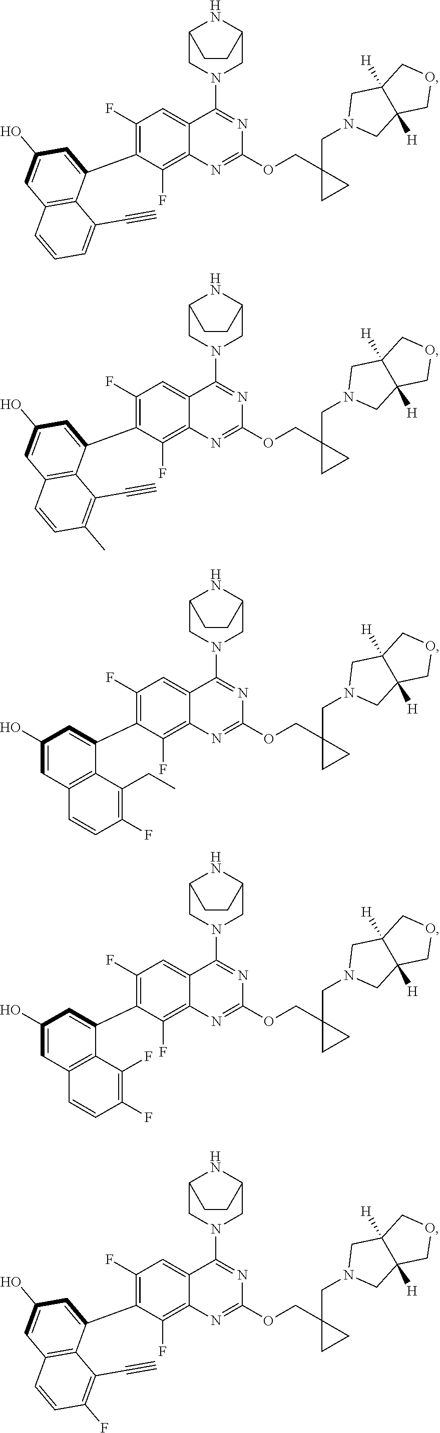

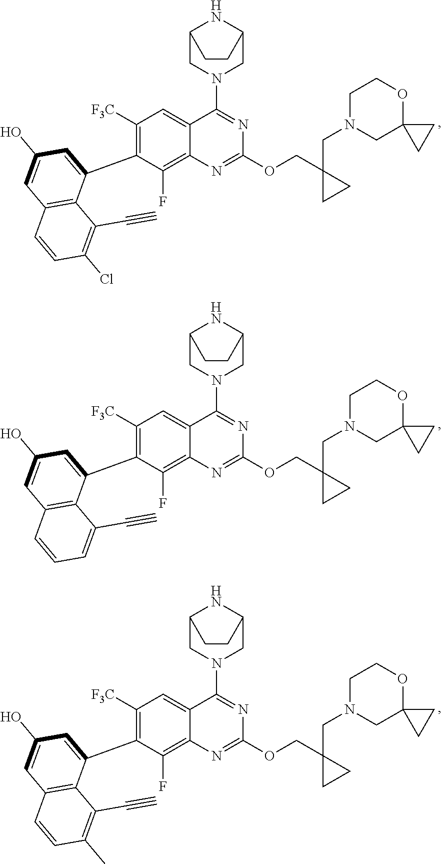

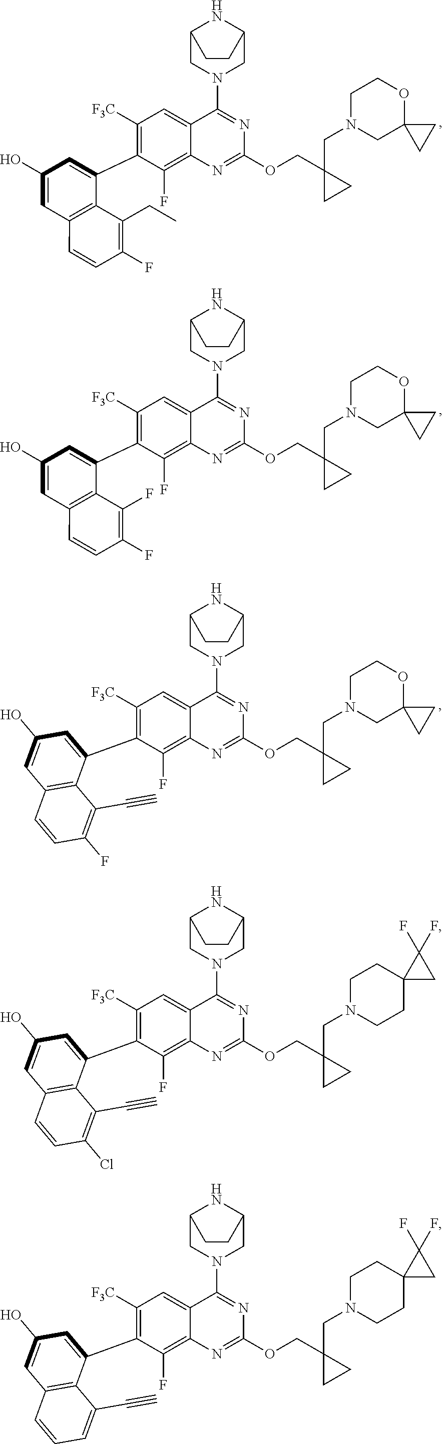

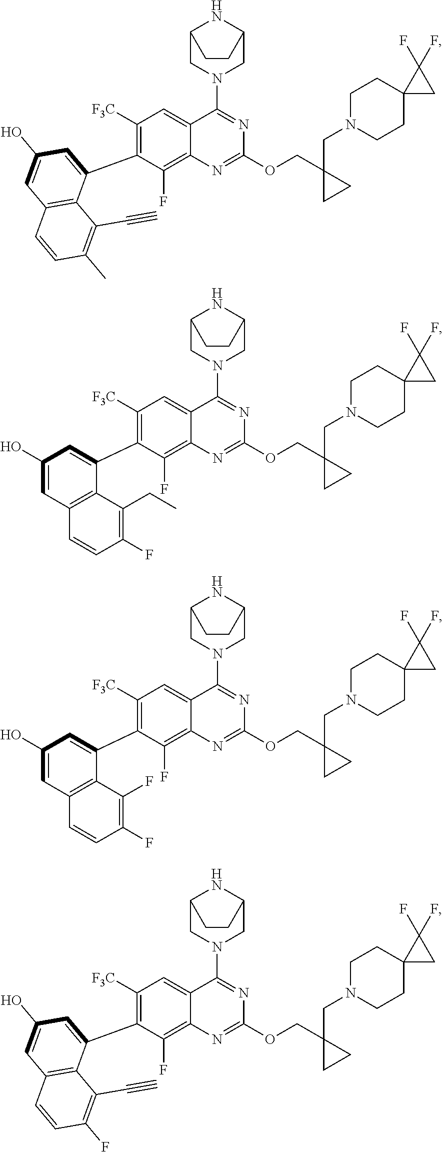

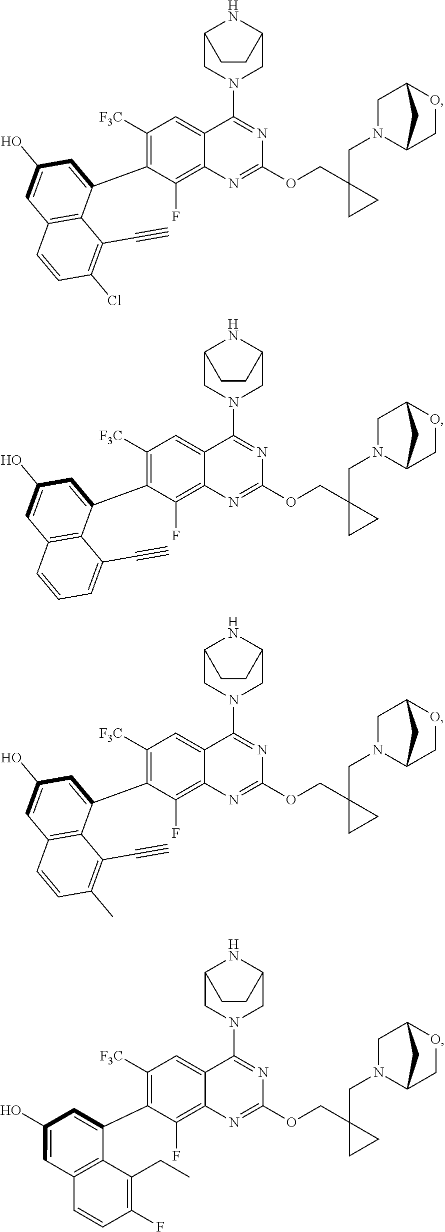

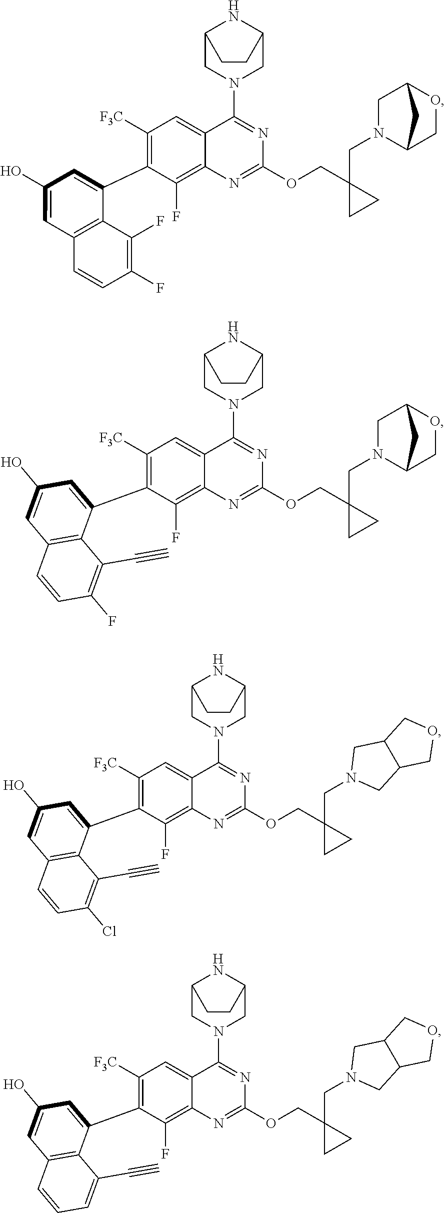

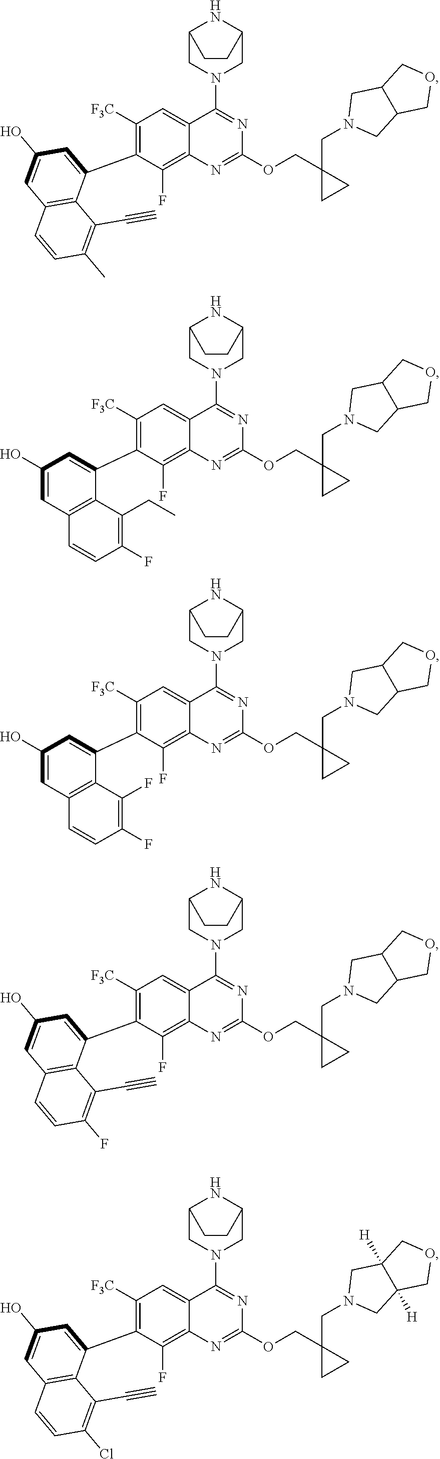

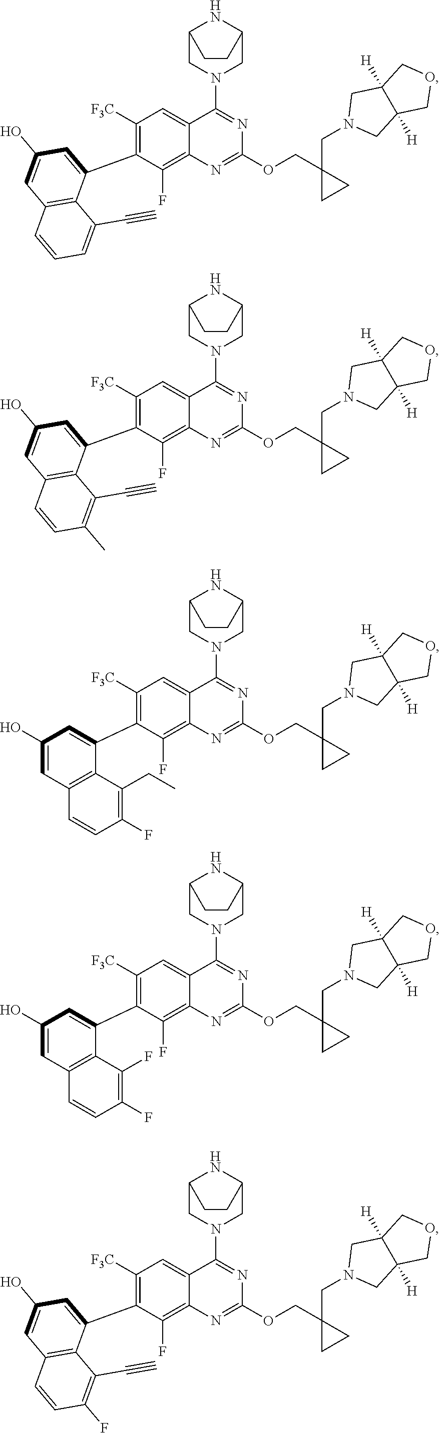

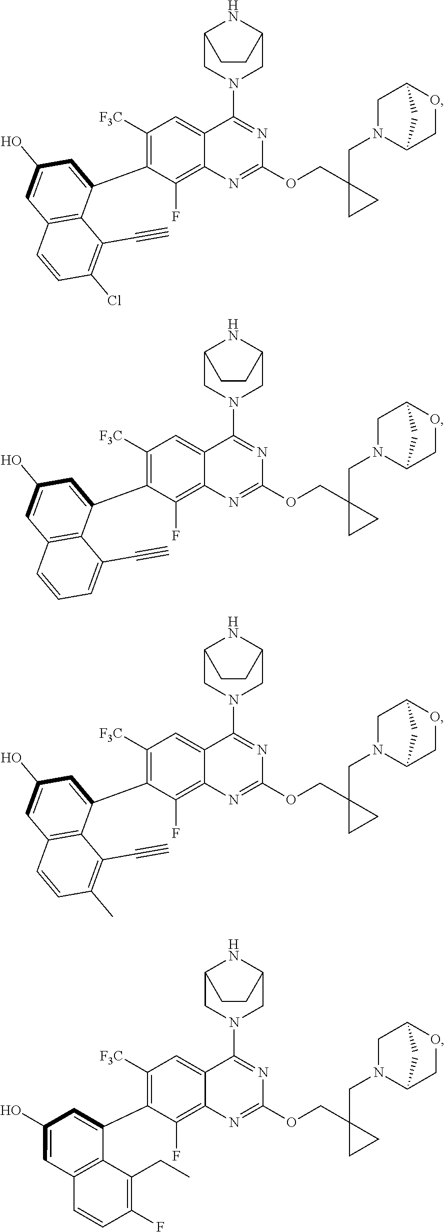

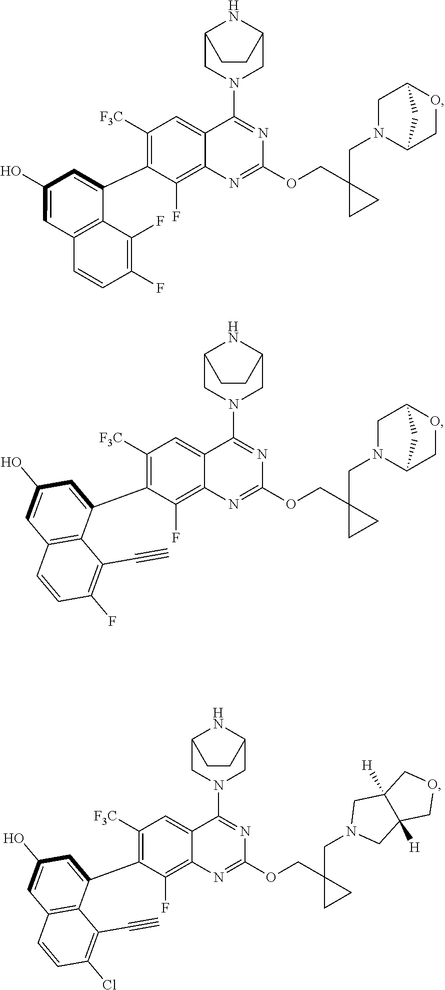

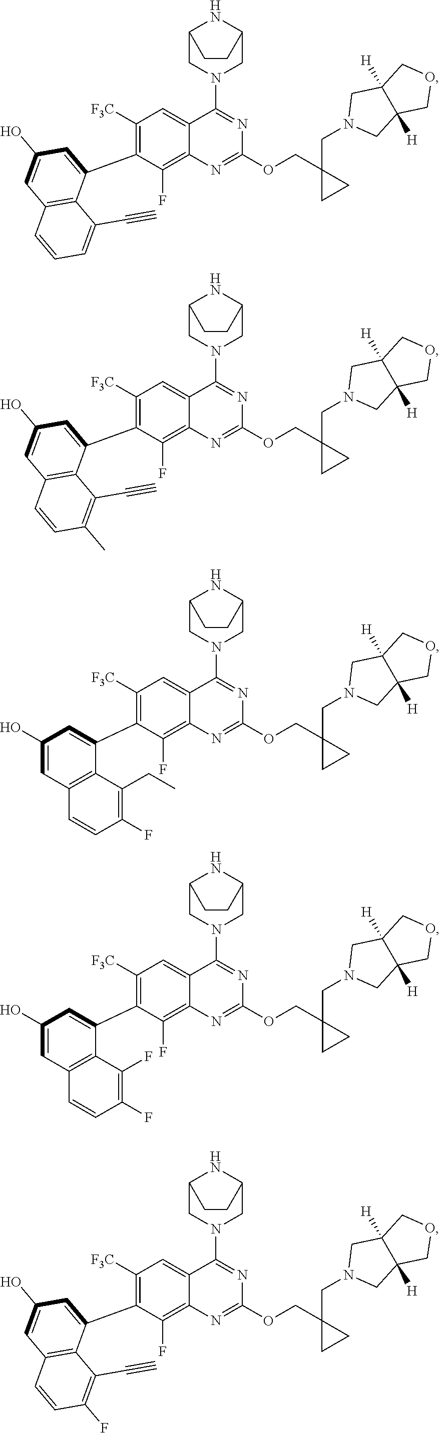

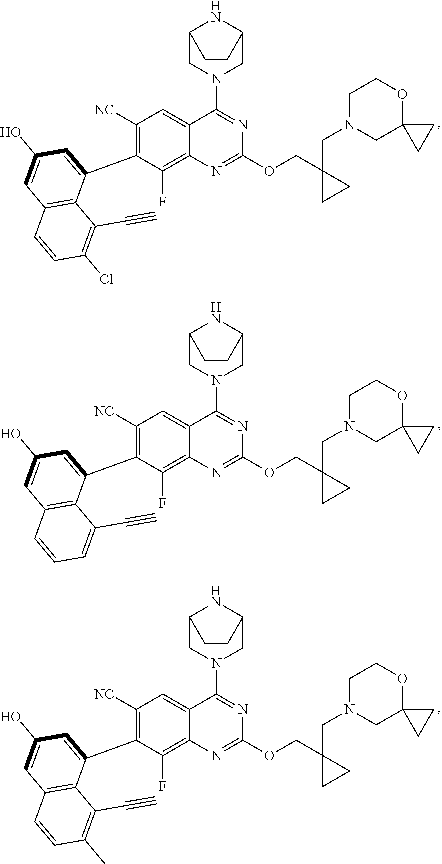

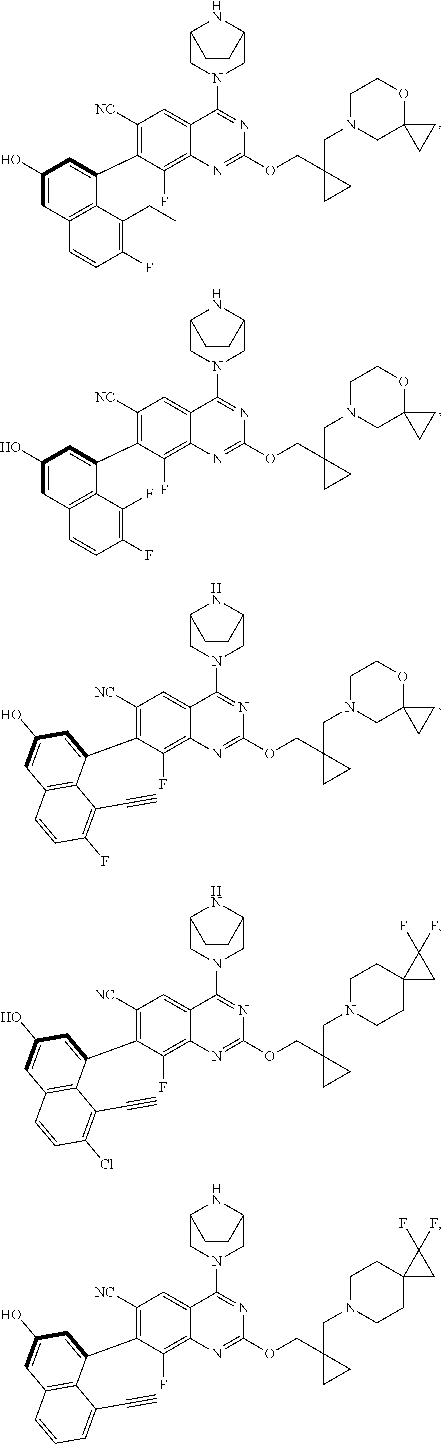

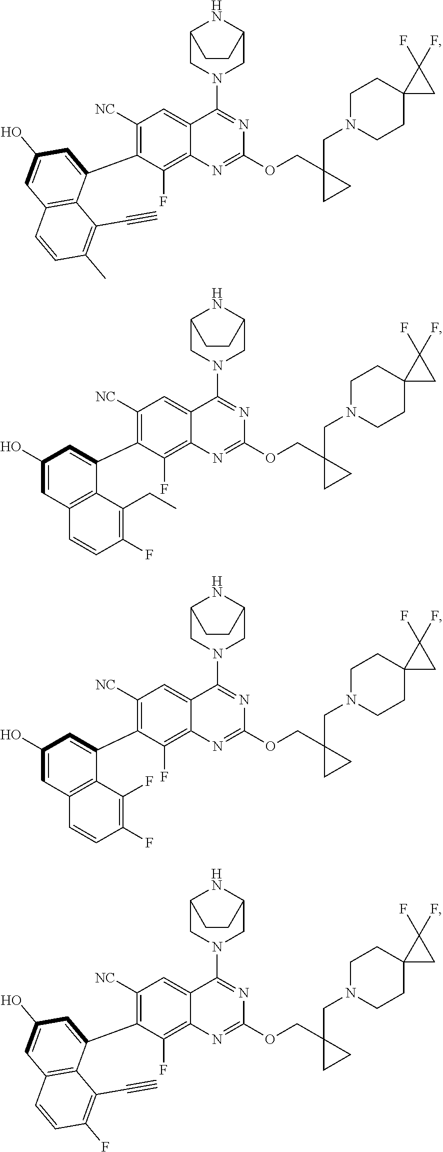

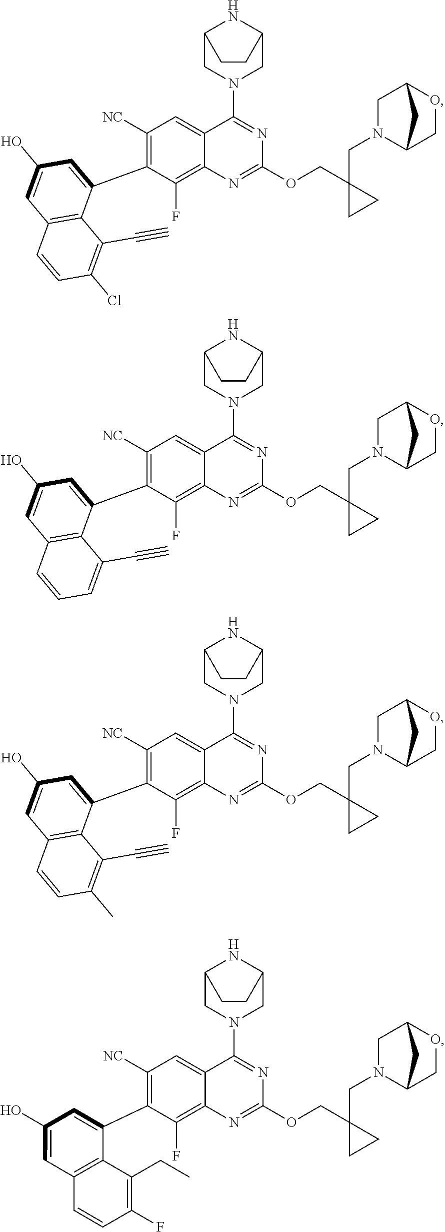

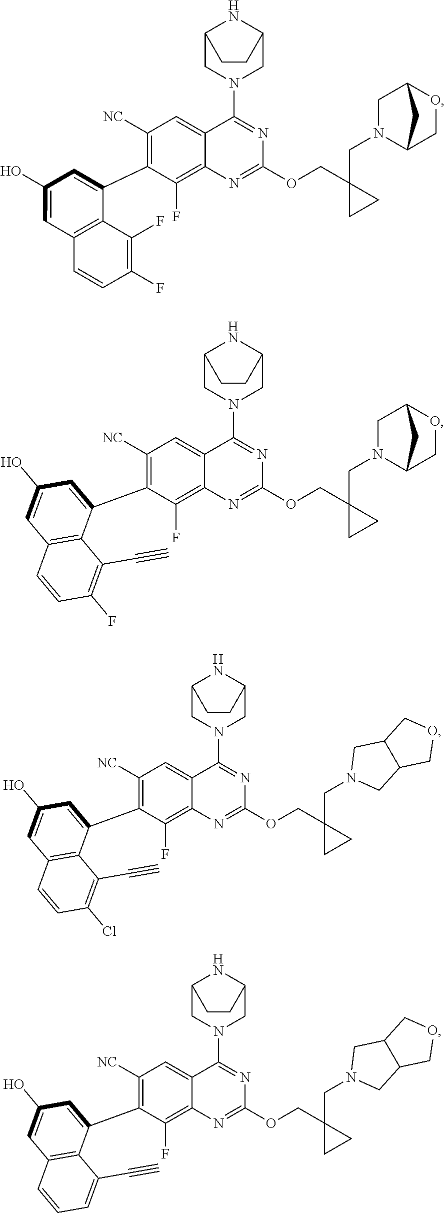

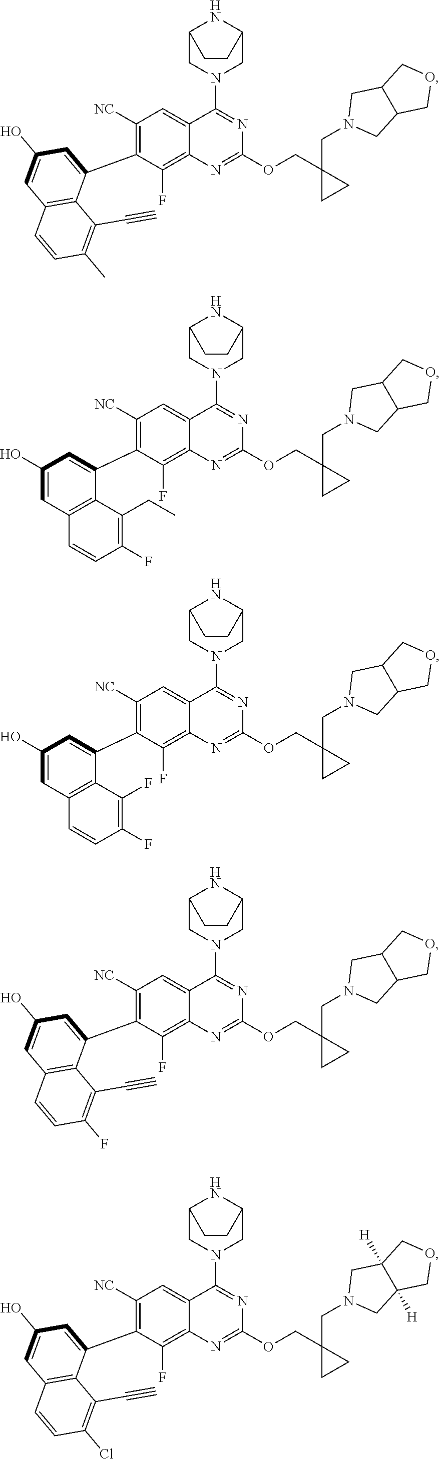

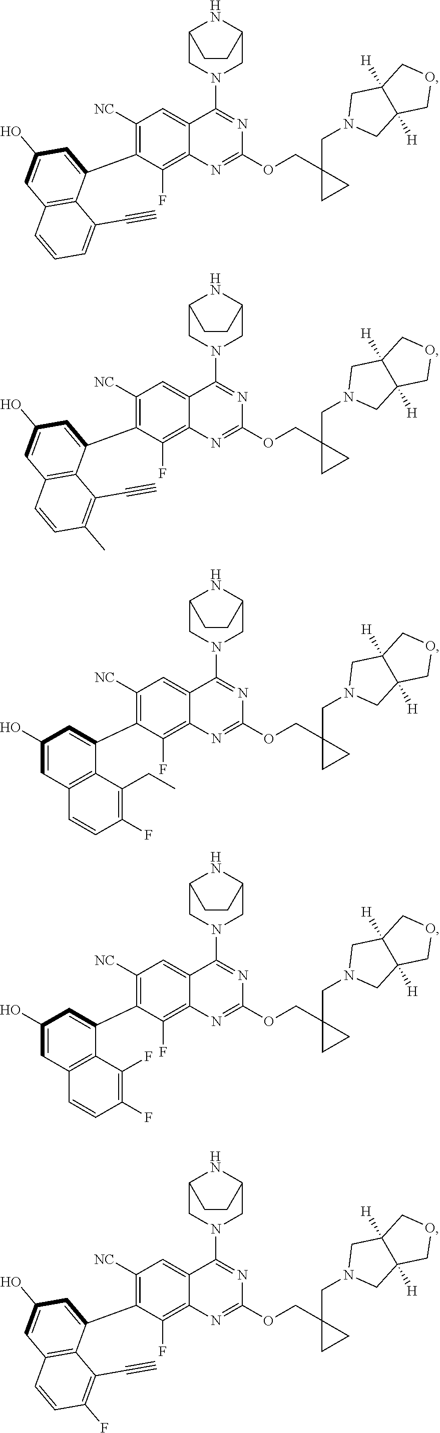

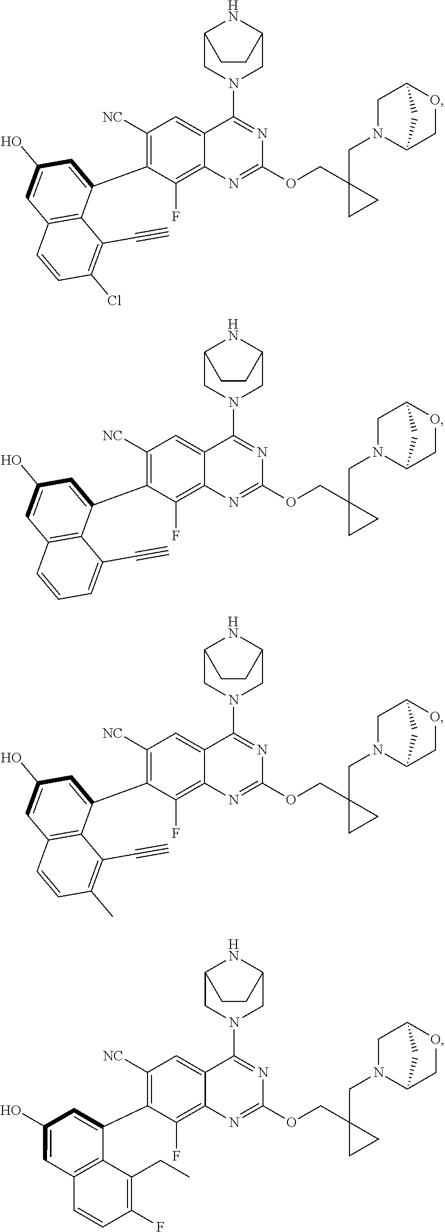

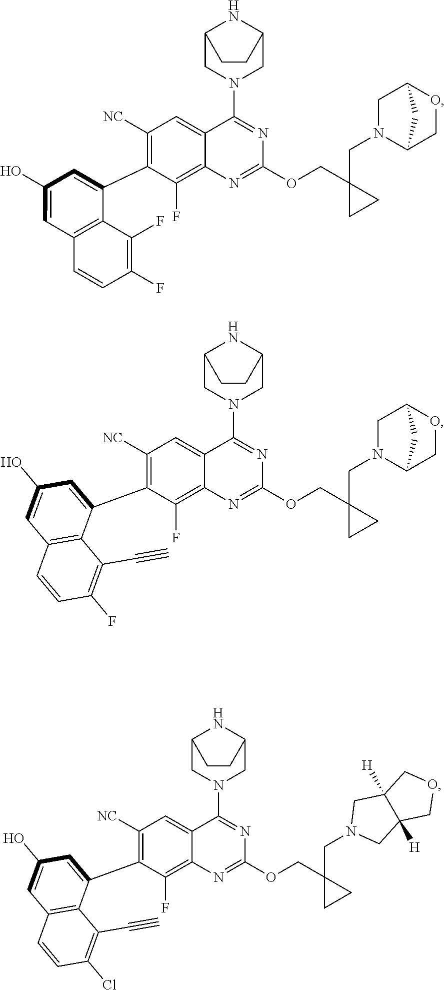

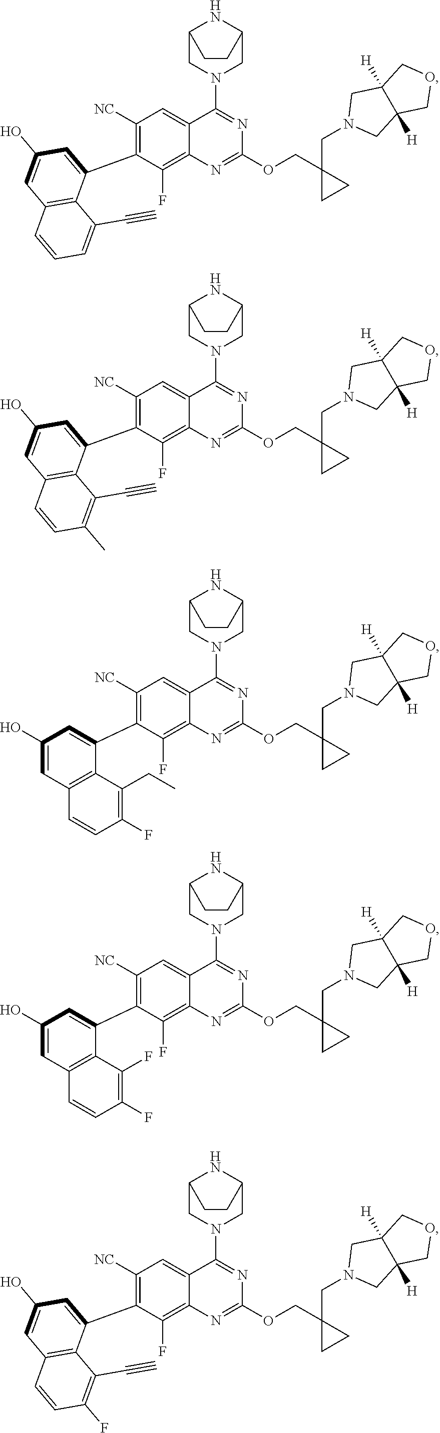

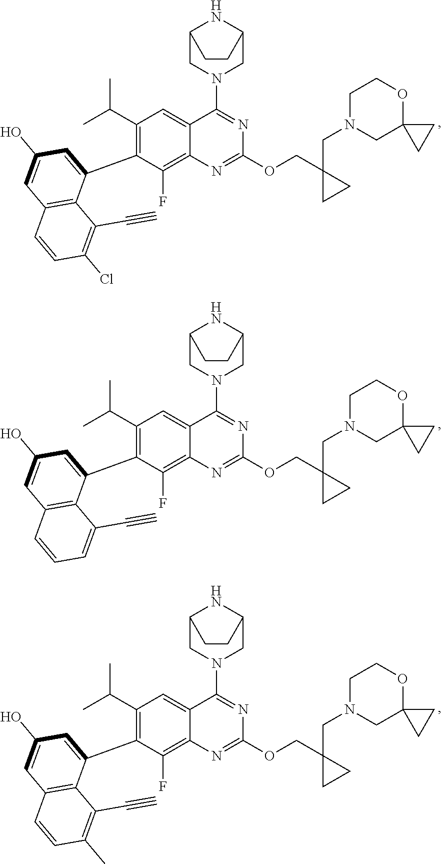

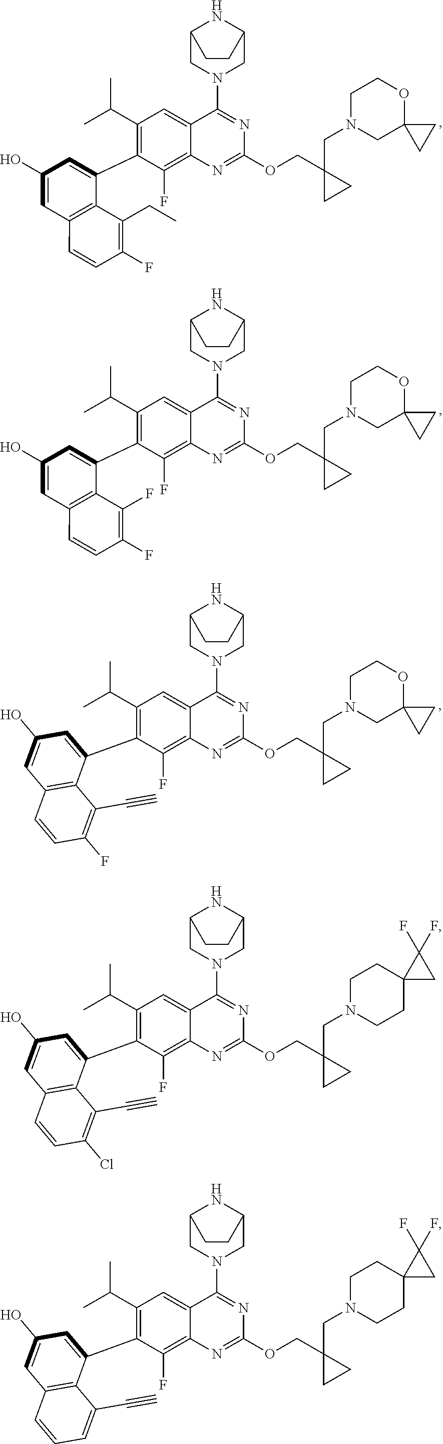

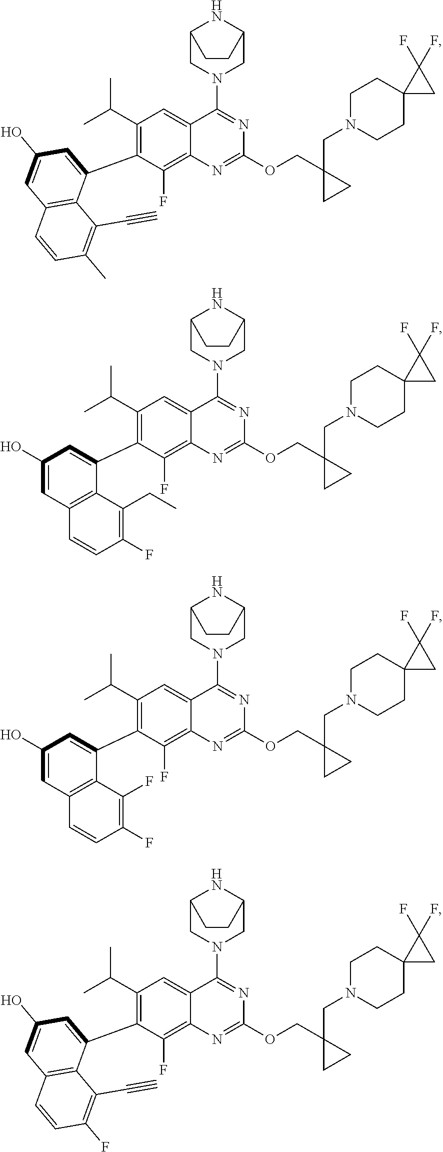

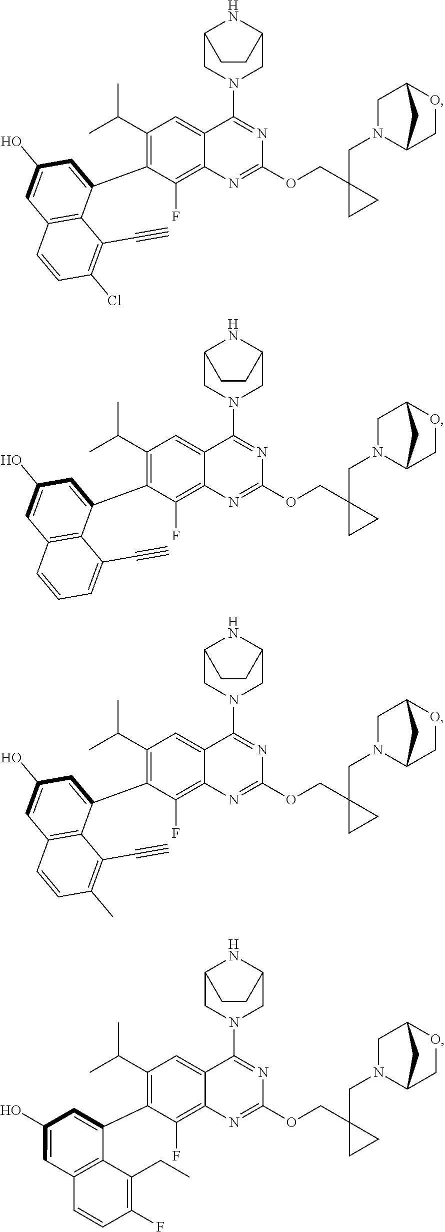

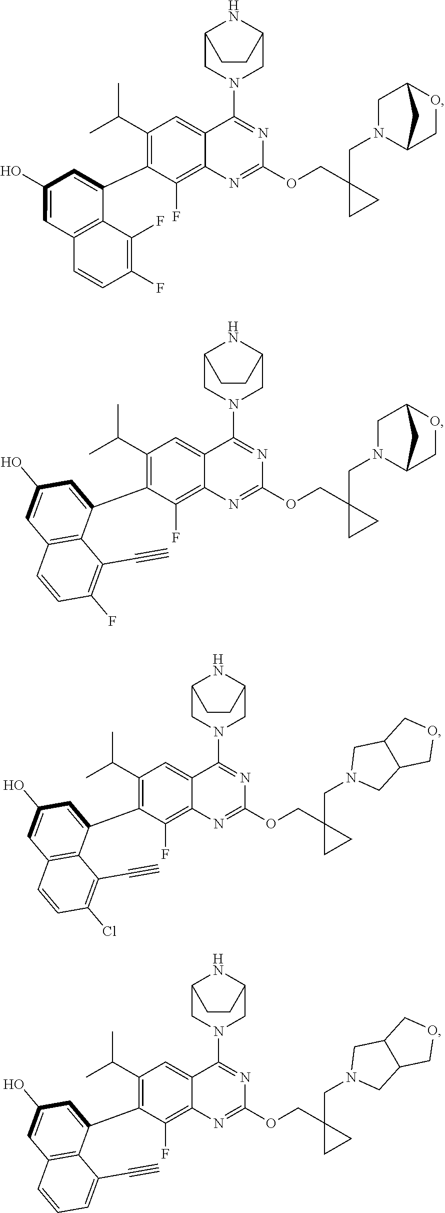

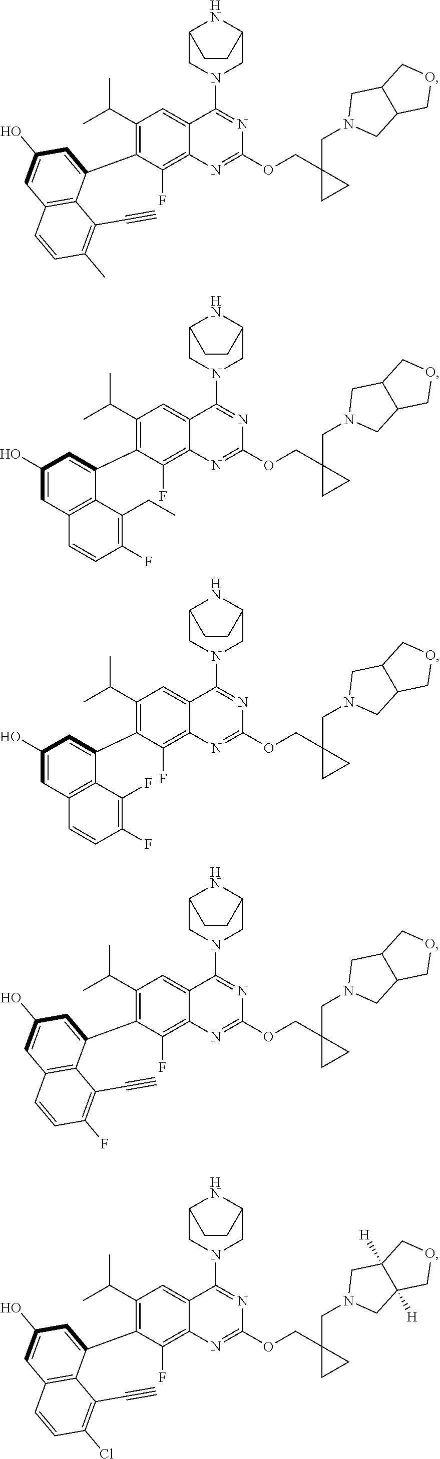

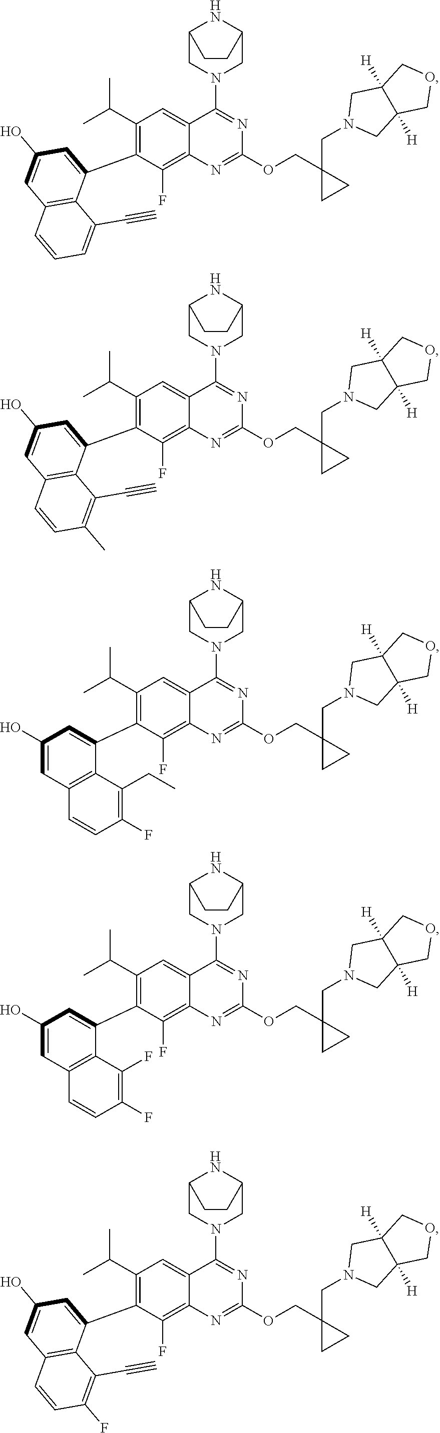

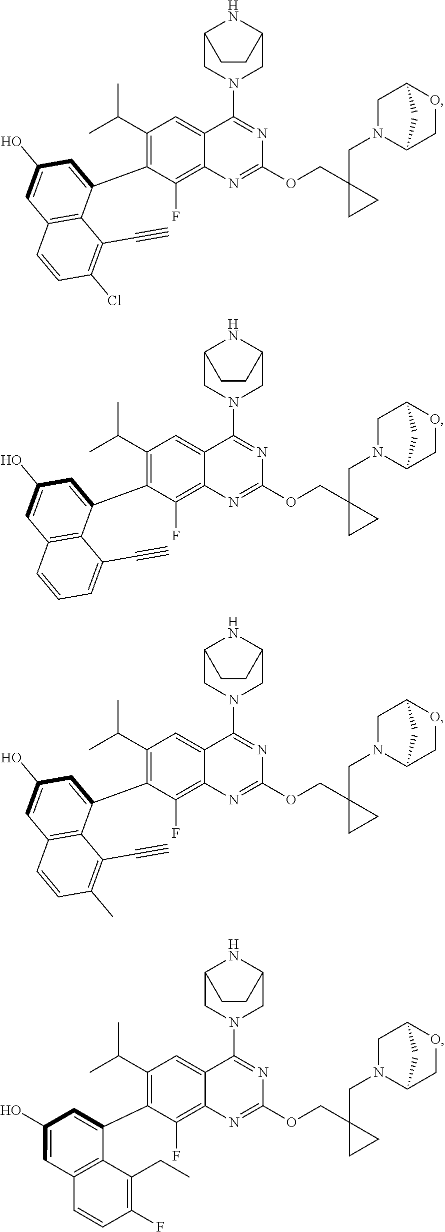

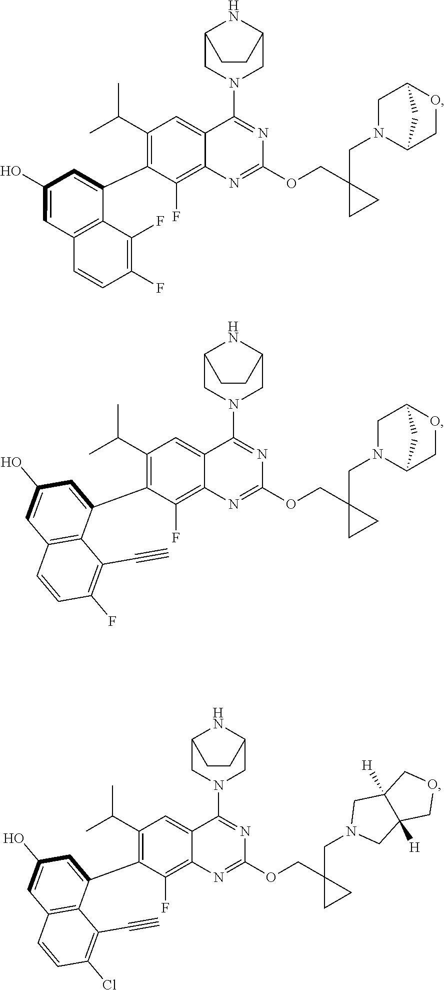

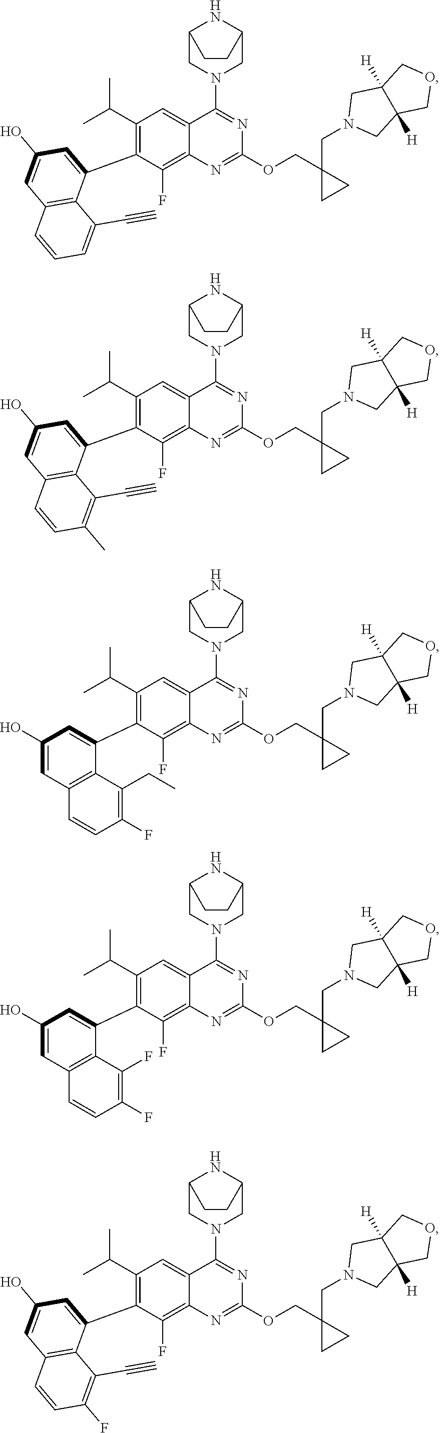

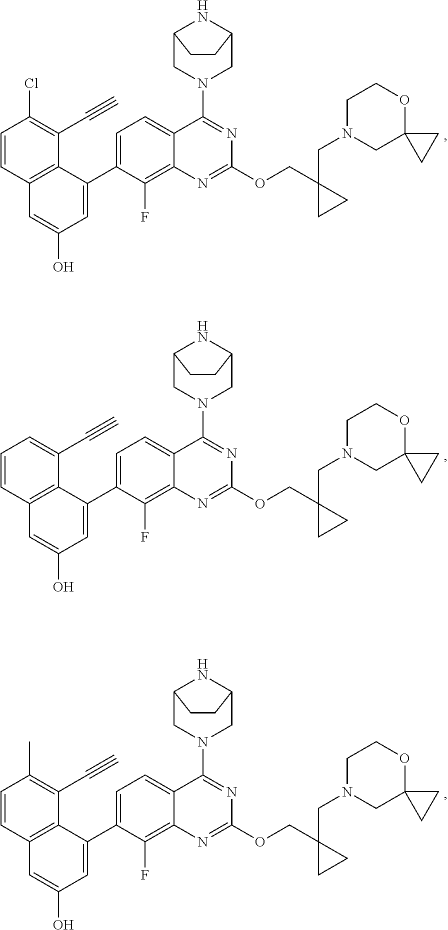 [SMILES: C#Cc1c(Cl)ccc2cc(O)cc(-c3ccc4c(N5CC6CCC(C5)N6)nc(OCC5(CN6CCOC7(CC7)C6)CC5)nc4c3F)c12 ; C#Cc1cccc2cc(O)cc(-c3ccc4c(N5CC6CCC(C5)N6)nc(OCC5(CN6CCOC7(CC7)C6)CC5)nc4c3F)c12 ; C#Cc1c(C)ccc2cc(O)cc(-c3ccc4c(N5CC6CCC(C5)N6)nc(OCC5(CN6CCOC7(CC7)C6)CC5)nc4c3F)c12]

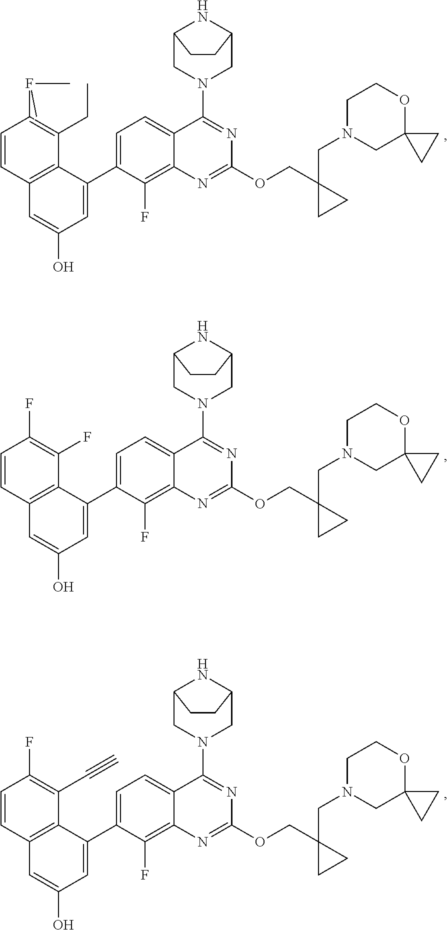

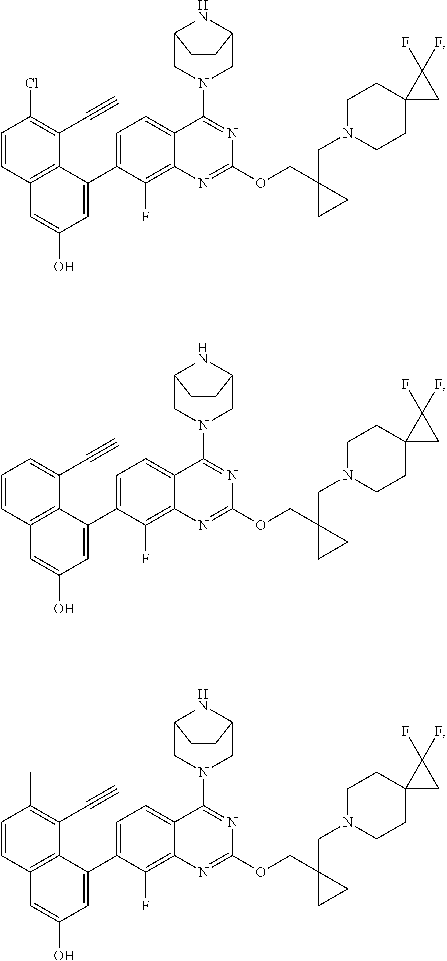 [SMILES: C#Cc1c(Cl)ccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; C#Cc1c(C)ccc2cc(O)cc(-c3ccc4c(N5CC6CCC(C5)N6)nc(OCC5(CN6CCC7(CC6)CC7(F)F)CC5)nc4c3F)c12]

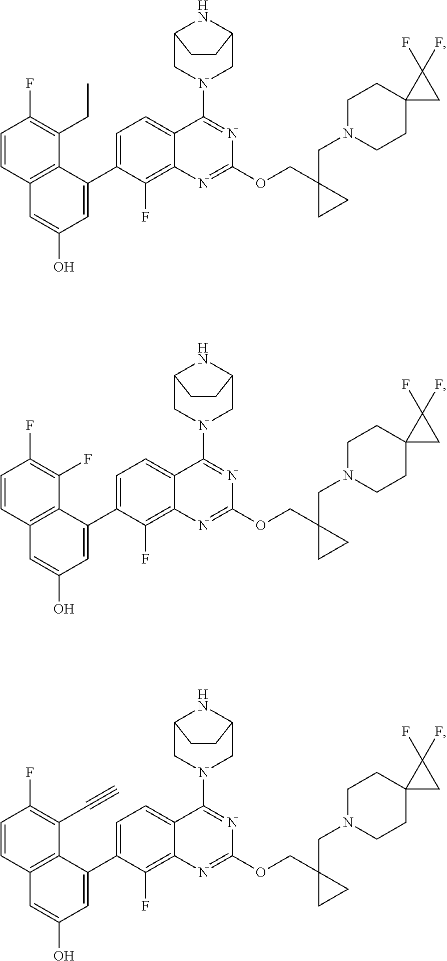 [SMILES: CCc1c(F)ccc2cc(O)cc(-c3ccc4c(N5CC6CCC(C5)N6)nc(OCC5(CN6CCC7(CC6)CC7(F)F)CC5)nc4c3F)c12 ; Oc1cc(-c2ccc3c(N4CC5CCC(C4)N5)nc(OCC4(CN5CCC6(CC5)CC6(F)F)CC4)nc3c2F)c2c(F)c(F)ccc2c1 ; C#Cc1c(F)ccc2cc(O)cc(-c3ccc4c(N5CC6CCC(C5)N6)nc(OCC5(CN6CCC7(CC6)CC7(F)F)CC5)nc4c3F)c12]

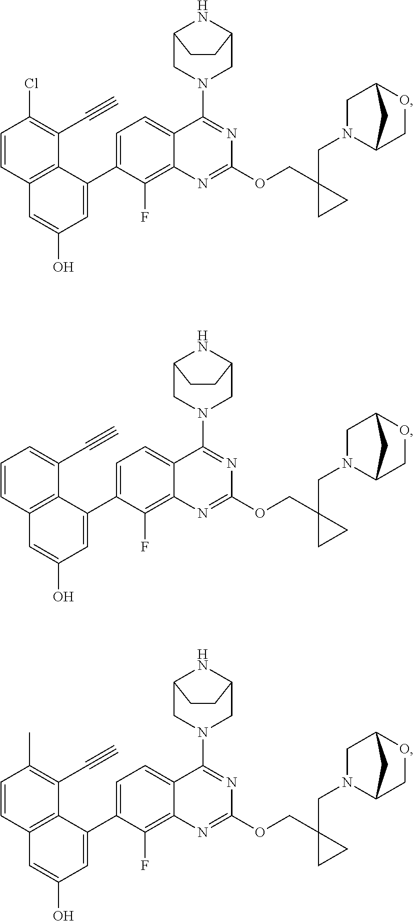 [SMILES: C#Cc1c(Cl)ccc2cc(O)cc(-c3ccc4c(N5CC6CCC(C5)N6)nc(OCC5(CN6C[C@H]7C[C@@H]6CO7)CC5)nc4c3F)c12 ; C#Cc1cccc2cc(O)cc(-c3ccc4c(N5CC6CCC(C5)N6)nc(OCC5(CN6C[C@H]7C[C@@H]6CO7)CC5)nc4c3F)c12 ; C#Cc1c(C)ccc2cc(O)cc(-c3ccc4c(N5CC6CCC(C5)N6)nc(OCC5(CN6C[C@H]7C[C@@H]6CO7)CC5)nc4c3F)c12]

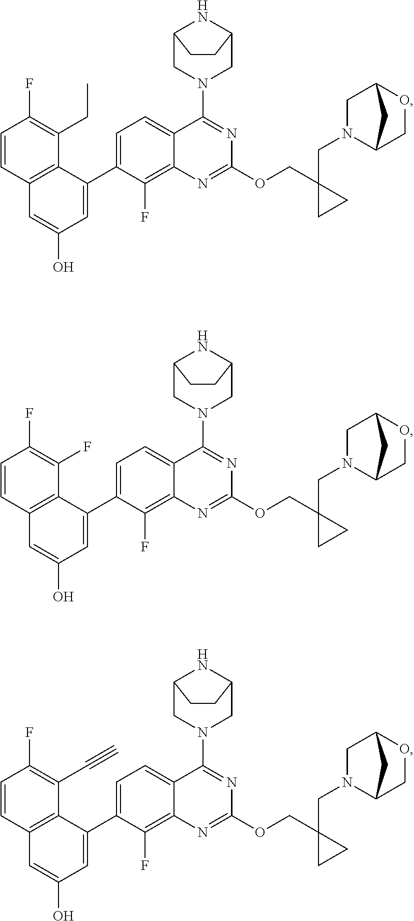 [SMILES: CCc1c(F)ccc2cc(O)cc(-c3ccc4c(N5CC6CCC(C5)N6)nc(OCC5(CN6C[C@H]7C[C@@H]6CO7)CC5)nc4c3F)c12 ; Oc1cc(-c2ccc3c(N4CC5CCC(C4)N5)nc(OCC4(CN5C[C@H]6C[C@@H]5CO6)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7C[C@@H]6CO7)CC5)nc4c3F)c12]

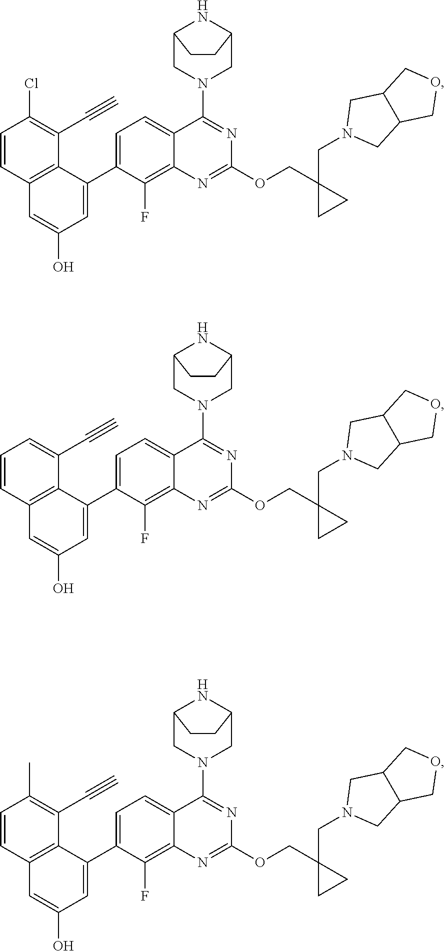 [SMILES: C#Cc1c(Cl)ccc2cc(O)cc(-c3ccc4c(N5CC6CCC(C5)N6)nc(OCC5(CN6CC7COCC7C6)CC5)nc4c3F)c12 ; C#Cc1cccc2cc(O)cc(-c3ccc4c(N5CC6CCC(C5)N6)nc(OCC5(CN6CC7COCC7C6)CC5)nc4c3F)c12 ; C#Cc1c(C)ccc2cc(O)cc(-c3ccc4c(N5CC6CCC(C5)N6)nc(OCC5(CN6CC7COCC7C6)CC5)nc4c3F)c12]

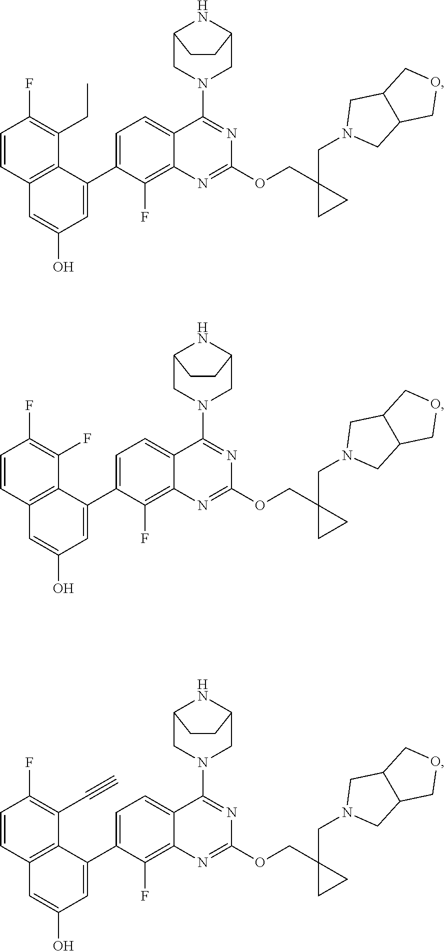 [SMILES: CCc1c(F)ccc2cc(O)cc(-c3ccc4c(N5CC6CCC(C5)N6)nc(OCC5(CN6CC7COCC7C6)CC5)nc4c3F)c12 ; Oc1cc(-c2ccc3c(N4CC5CCC(C4)N5)nc(OCC4(CN5CC6COCC6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6CC7COCC7C6)CC5)nc4c3F)c12]

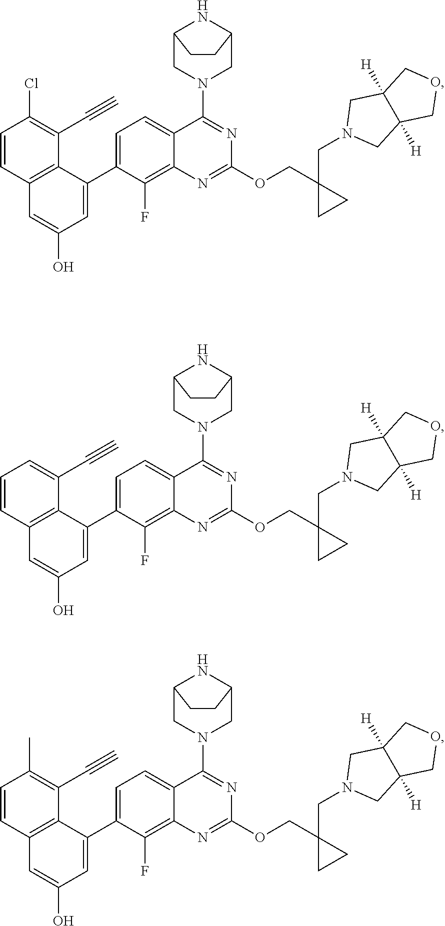 [SMILES: C#Cc1c(Cl)ccc2cc(O)cc(-c3ccc4c(N5CC6CCC(C5)N6)nc(OCC5(CN6C[C@H]7COC[C@H]7C6)CC5)nc4c3F)c12 ; C#Cc1cccc2cc(O)cc(-c3ccc4c(N5CC6CCC(C5)N6)nc(OCC5(CN6C[C@H]7COC[C@H]7C6)CC5)nc4c3F)c12 ; C#Cc1c(C)ccc2cc(O)cc(-c3ccc4c(N5CC6CCC(C5)N6)nc(OCC5(CN6C[C@H]7COC[C@H]7C6)CC5)nc4c3F)c12]

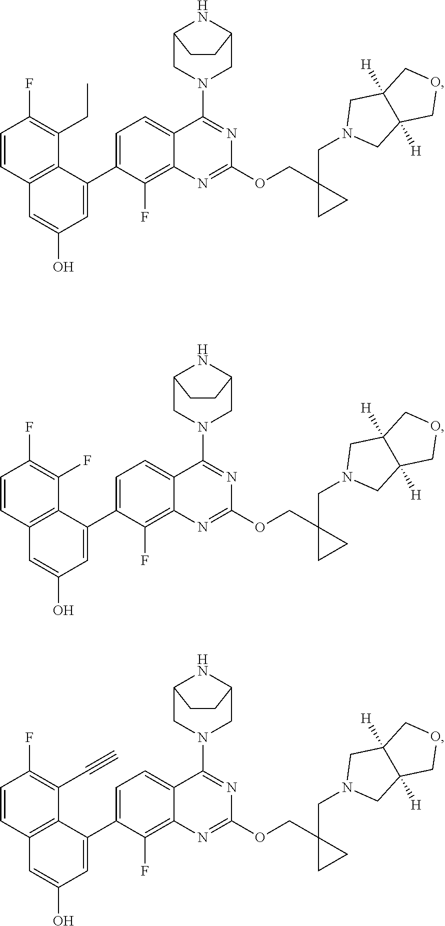 [SMILES: CCc1c(F)ccc2cc(O)cc(-c3ccc4c(N5CC6CCC(C5)N6)nc(OCC5(CN6C[C@H]7COC[C@H]7C6)CC5)nc4c3F)c12 ; Oc1cc(-c2ccc3c(N4CC5CCC(C4)N5)nc(OCC4(CN5C[C@H]6COC[C@H]6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7COC[C@H]7C6)CC5)nc4c3F)c12]

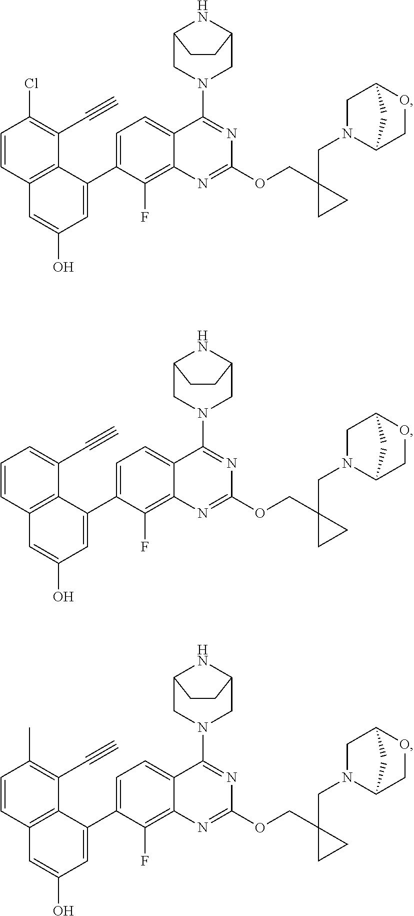 [SMILES: C#Cc1c(Cl)ccc2cc(O)cc(-c3ccc4c(N5CC6CCC(C5)N6)nc(OCC5(CN6C[C@@H]7C[C@H]6CO7)CC5)nc4c3F)c12 ; C#Cc1cccc2cc(O)cc(-c3ccc4c(N5CC6CCC(C5)N6)nc(OCC5(CN6C[C@@H]7C[C@H]6CO7)CC5)nc4c3F)c12 ; C#Cc1c(C)ccc2cc(O)cc(-c3ccc4c(N5CC6CCC(C5)N6)nc(OCC5(CN6C[C@@H]7C[C@H]6CO7)CC5)nc4c3F)c12]

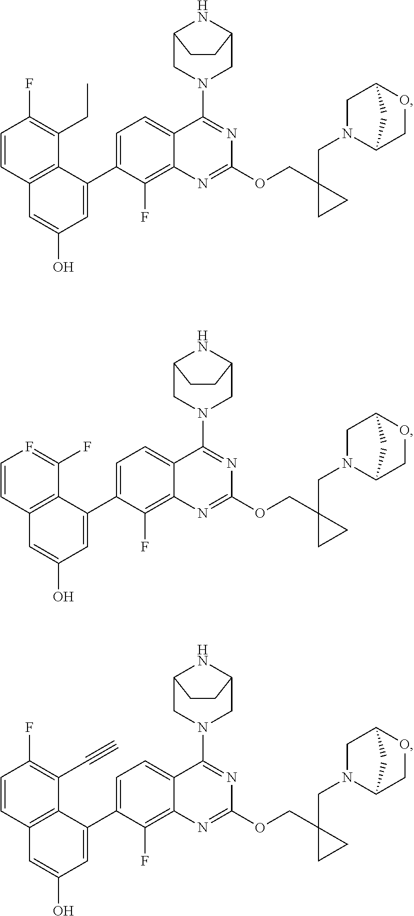

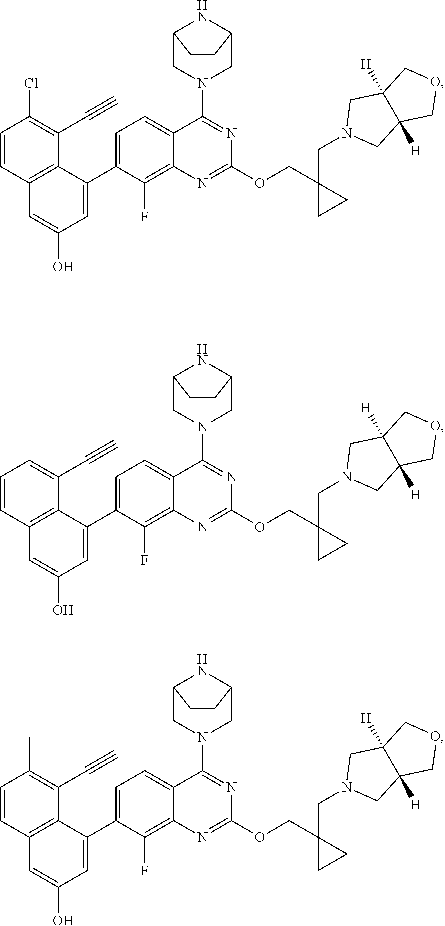 [SMILES: C#Cc1c(Cl)ccc2cc(O)cc(-c3ccc4c(N5CC6CCC(C5)N6)nc(OCC5(CN6C[C@H]7COC[C@@H]7C6)CC5)nc4c3F)c12 ; C#Cc1cccc2cc(O)cc(-c3ccc4c(N5CC6CCC(C5)N6)nc(OCC5(CN6C[C@H]7COC[C@@H]7C6)CC5)nc4c3F)c12 ; C#Cc1c(C)ccc2cc(O)cc(-c3ccc4c(N5CC6CCC(C5)N6)nc(OCC5(CN6C[C@H]7COC[C@@H]7C6)CC5)nc4c3F)c12]

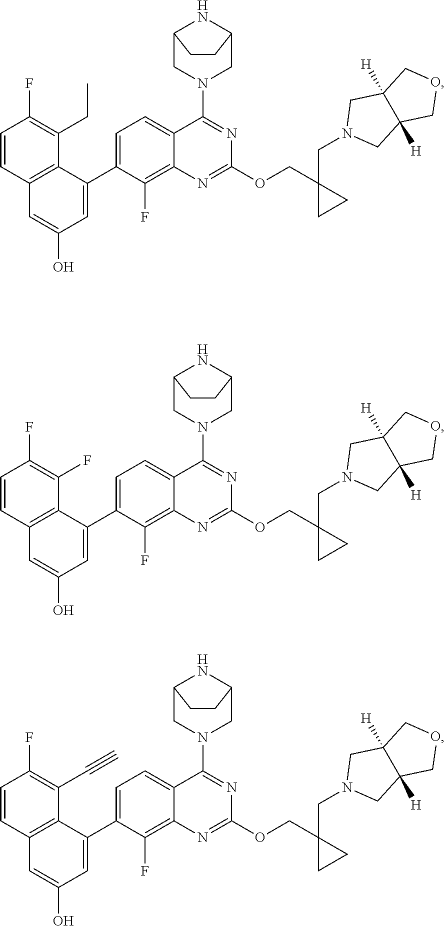 [SMILES: CCc1c(F)ccc2cc(O)cc(-c3ccc4c(N5CC6CCC(C5)N6)nc(OCC5(CN6C[C@H]7COC[C@@H]7C6)CC5)nc4c3F)c12 ; Oc1cc(-c2ccc3c(N4CC5CCC(C4)N5)nc(OCC4(CN5C[C@H]6COC[C@@H]6C5)CC4)nc3c2F)c2c(F)c(F)ccc2c1 ; C#Cc1c(F)ccc2cc(O)cc(-c3ccc4c(N5CC6CCC(C5)N6)nc(OCC5(CN6C[C@H]7COC[C@@H]7C6)CC5)nc4c3F)c12]

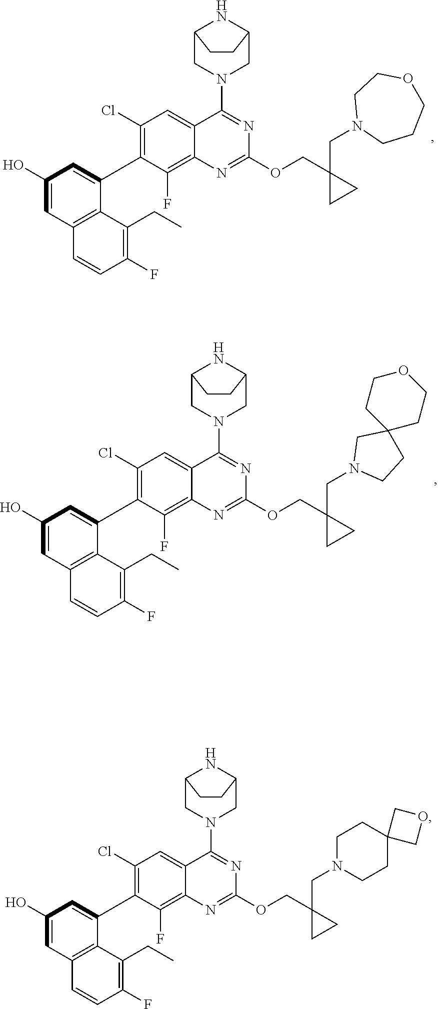

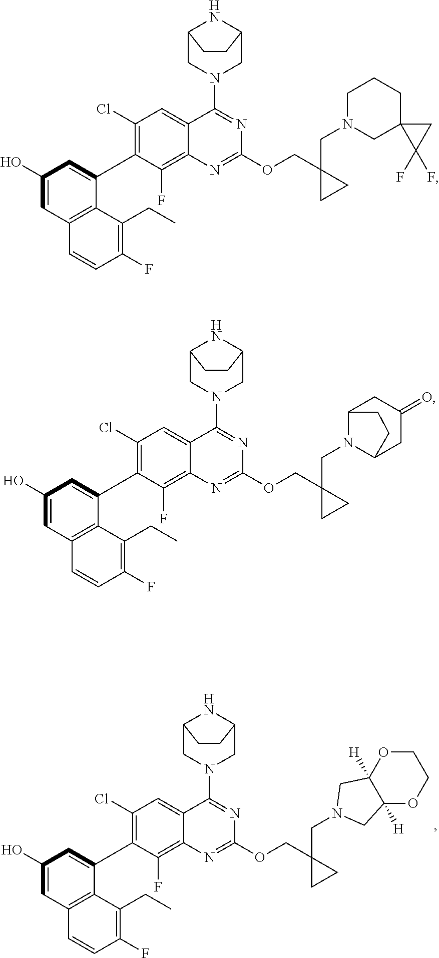

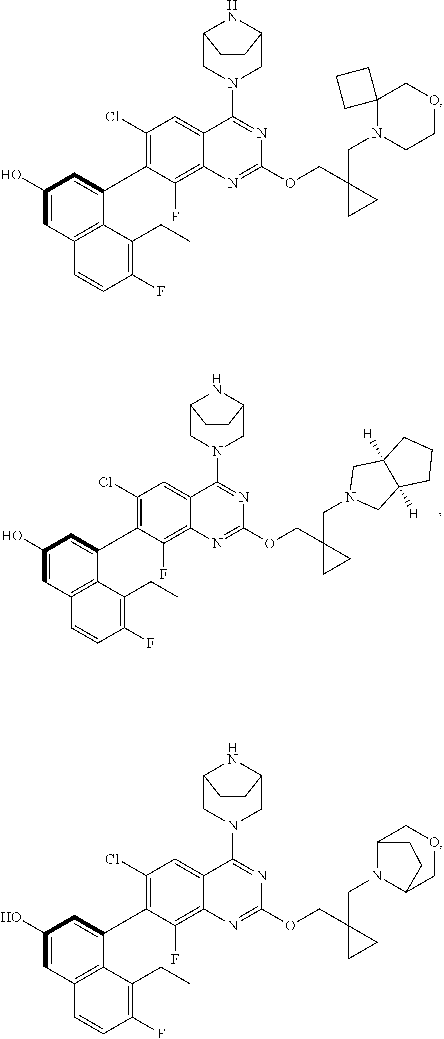

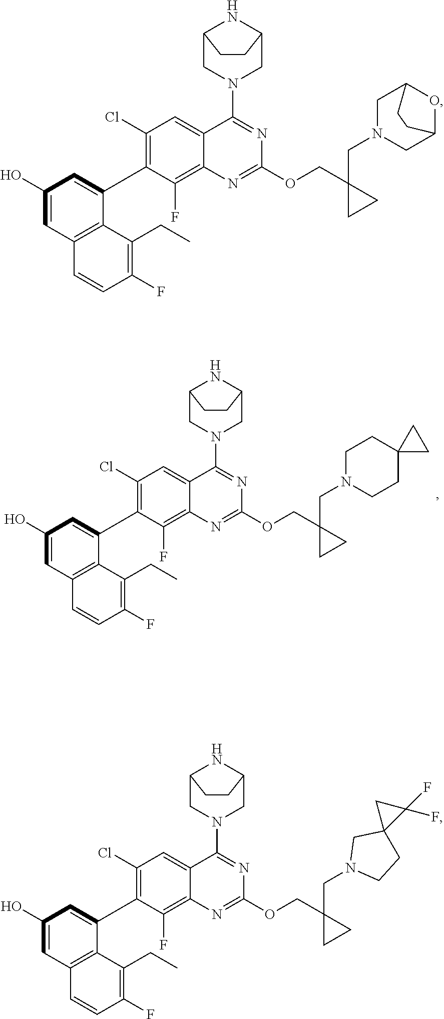

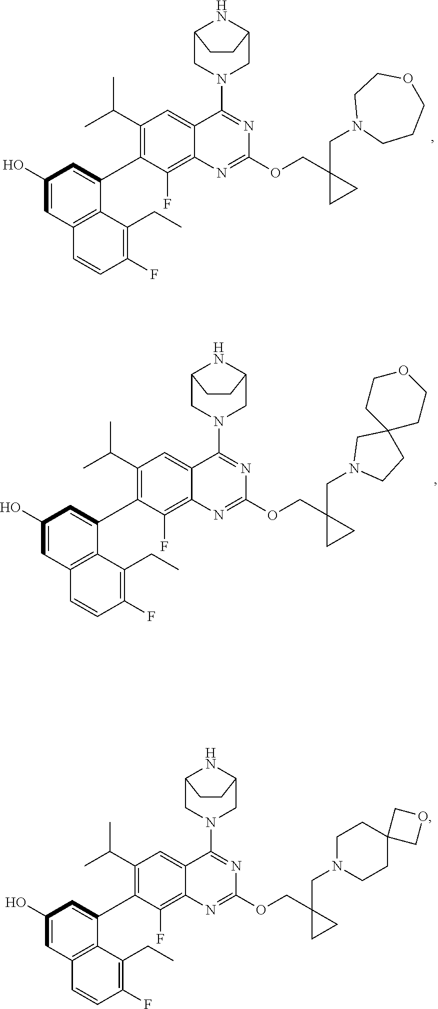

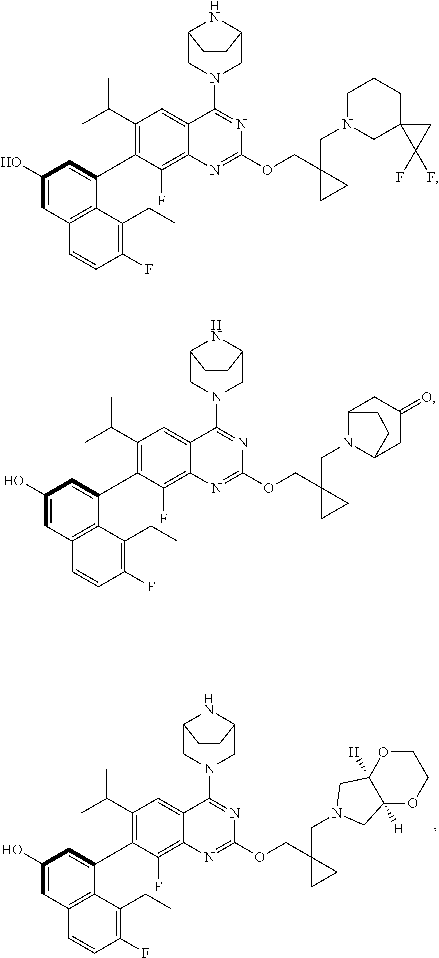

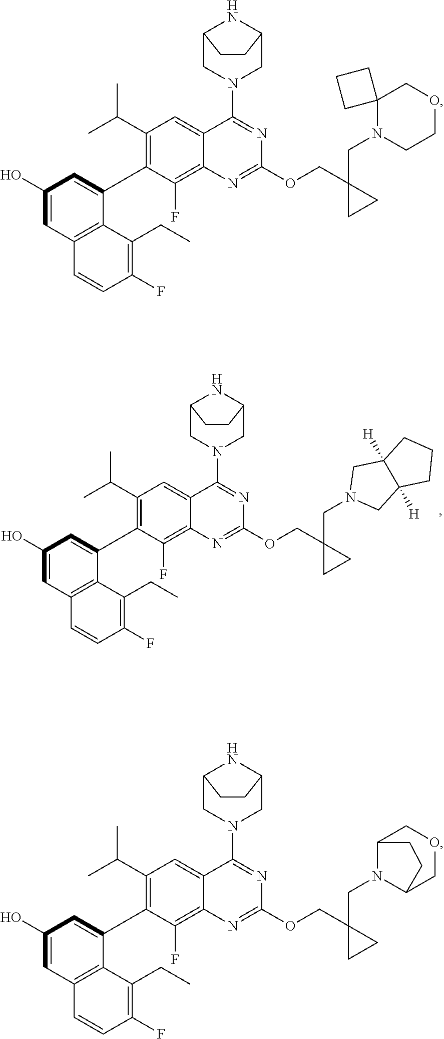

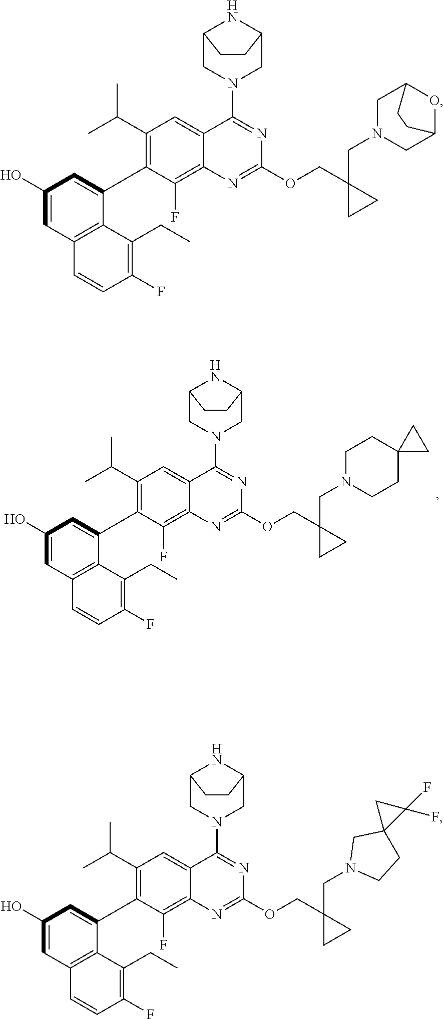

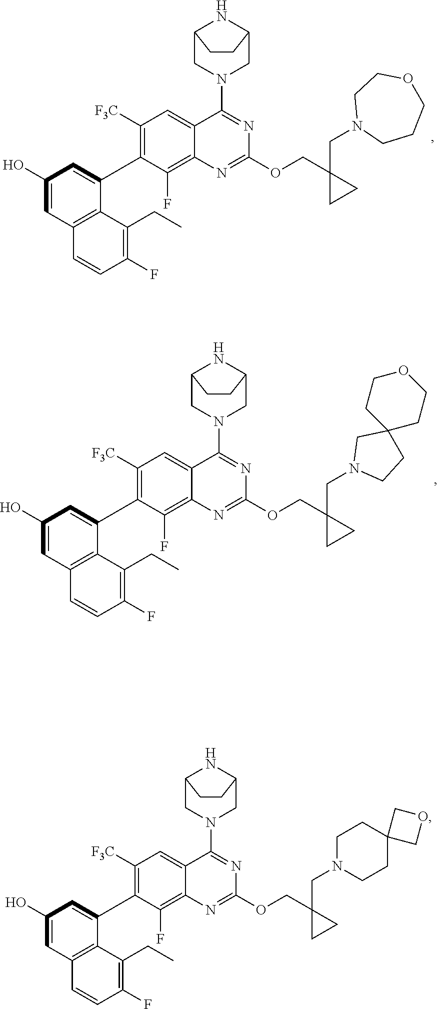

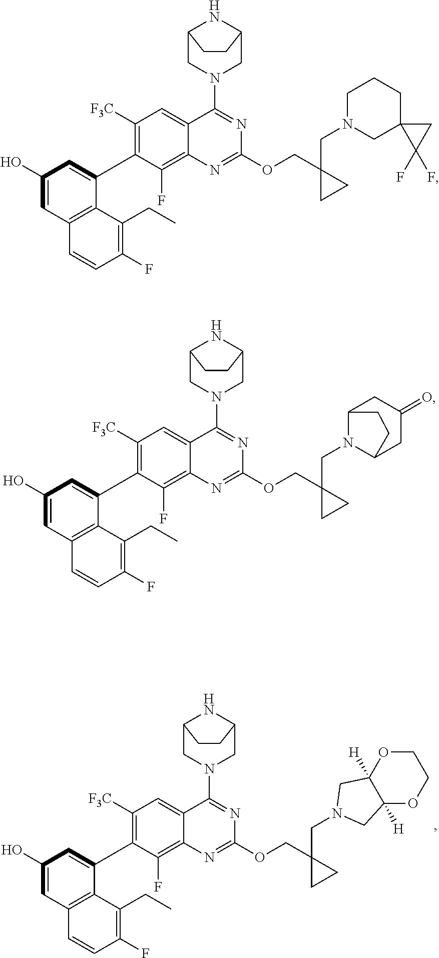

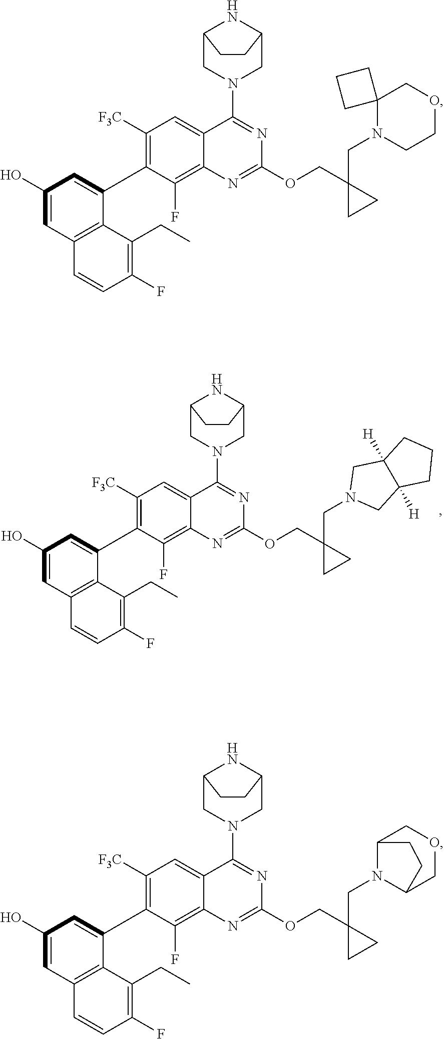

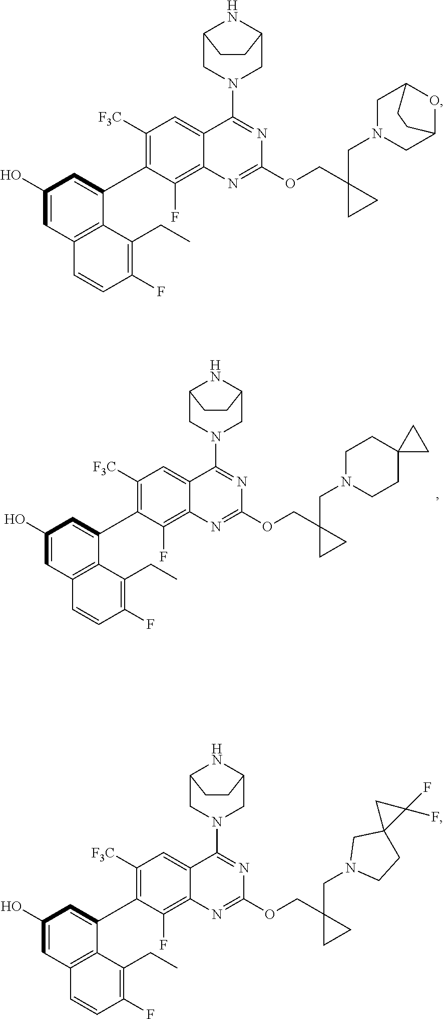

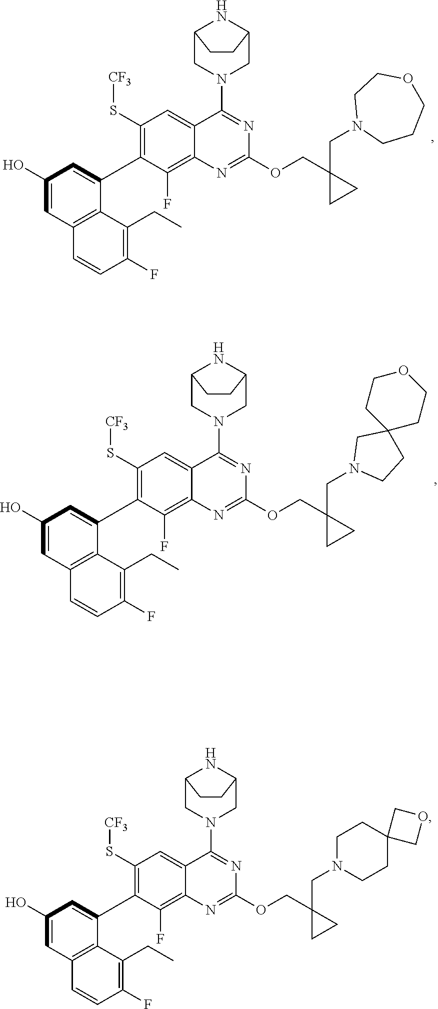

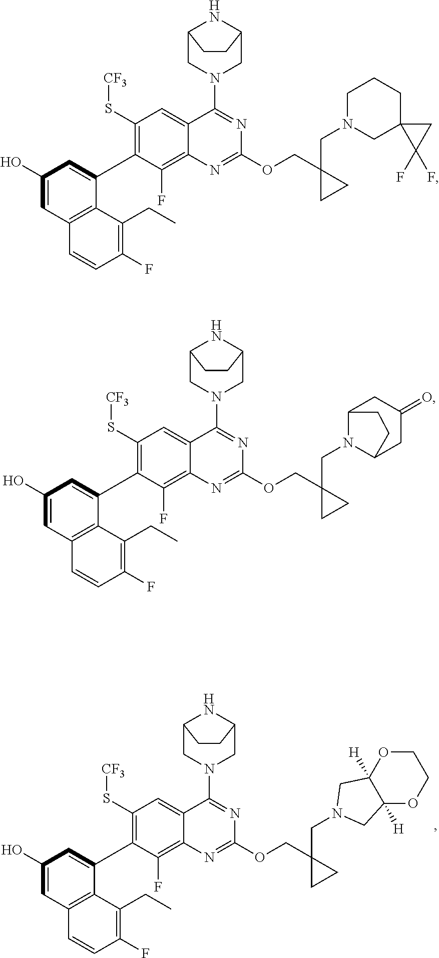

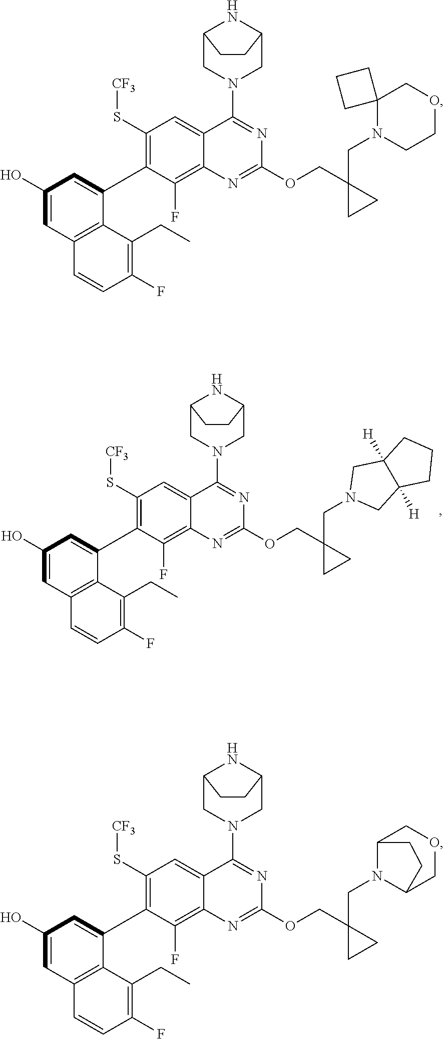

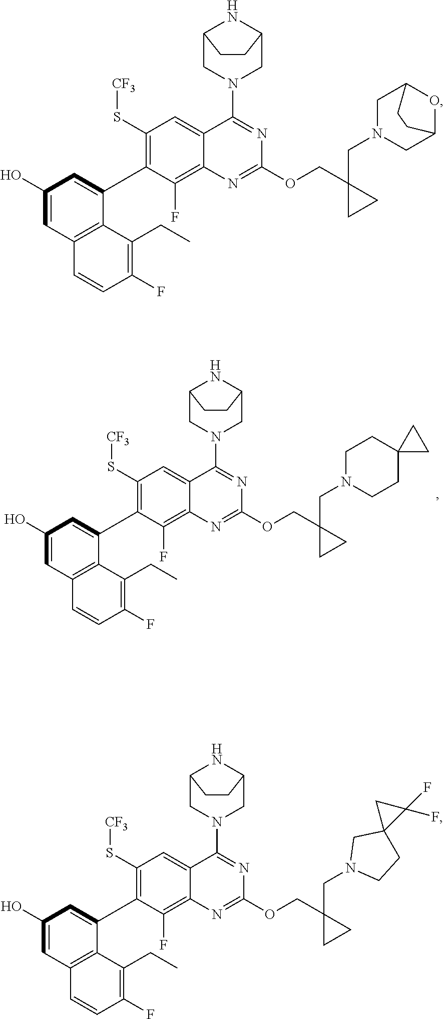

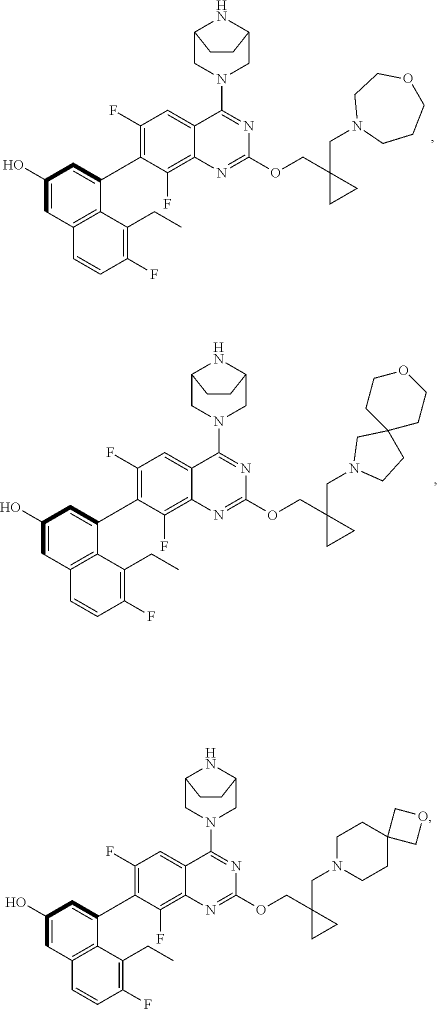

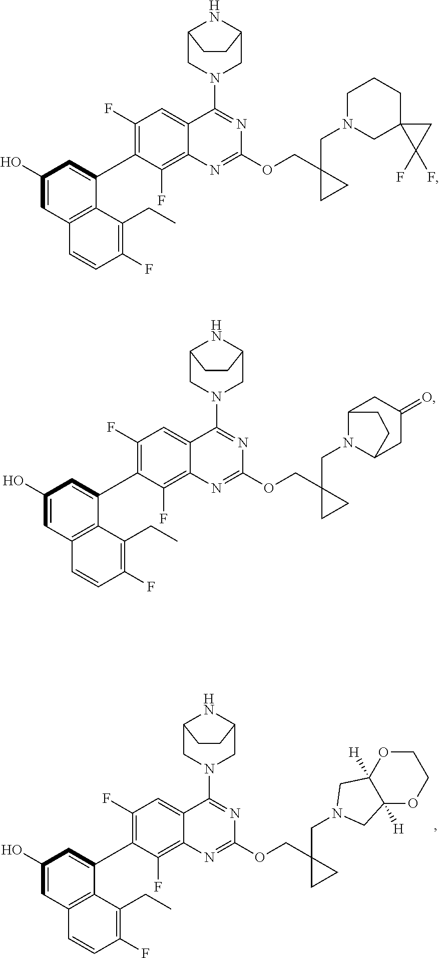

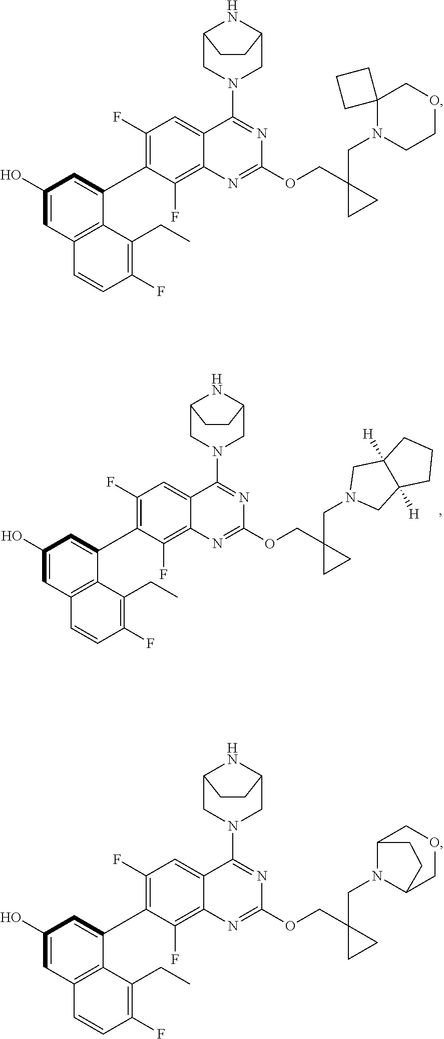

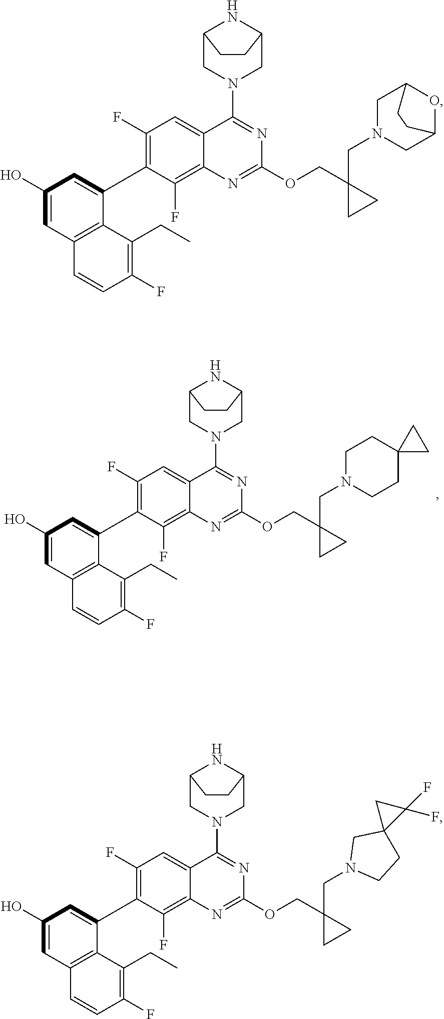

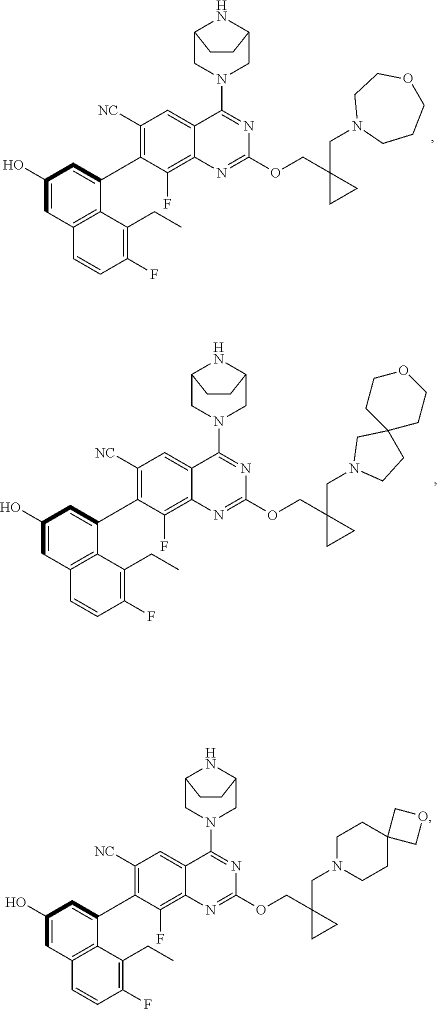

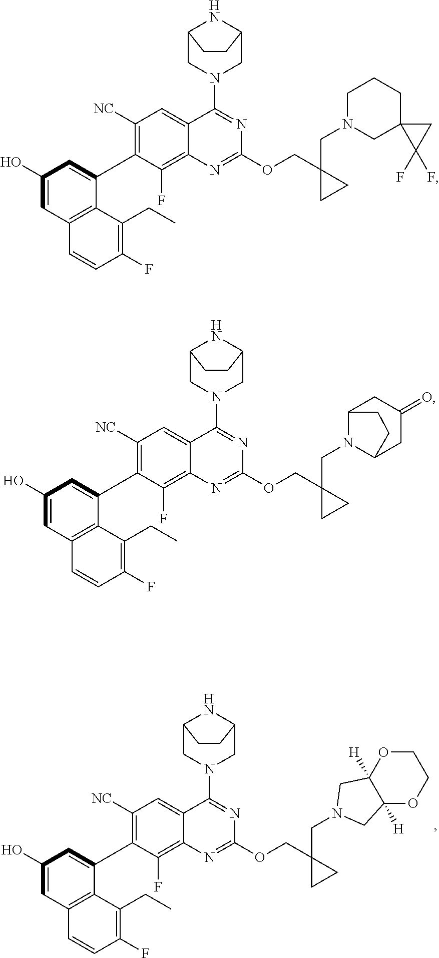

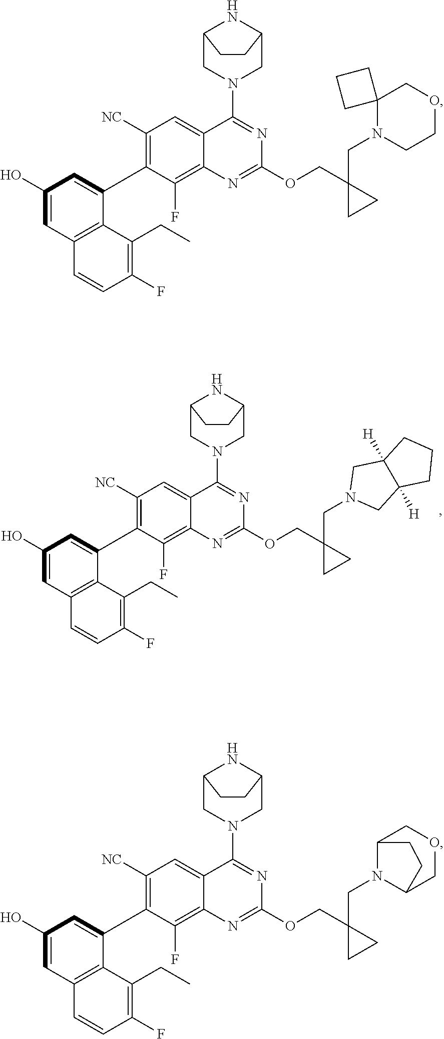

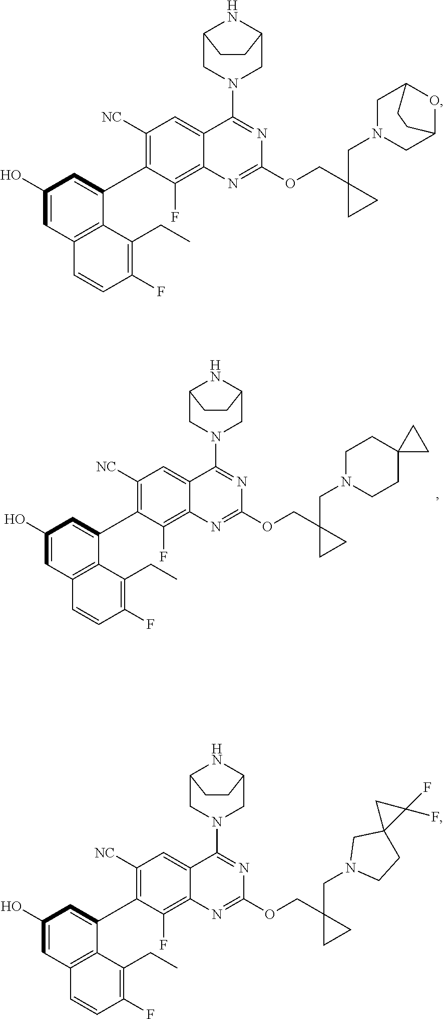

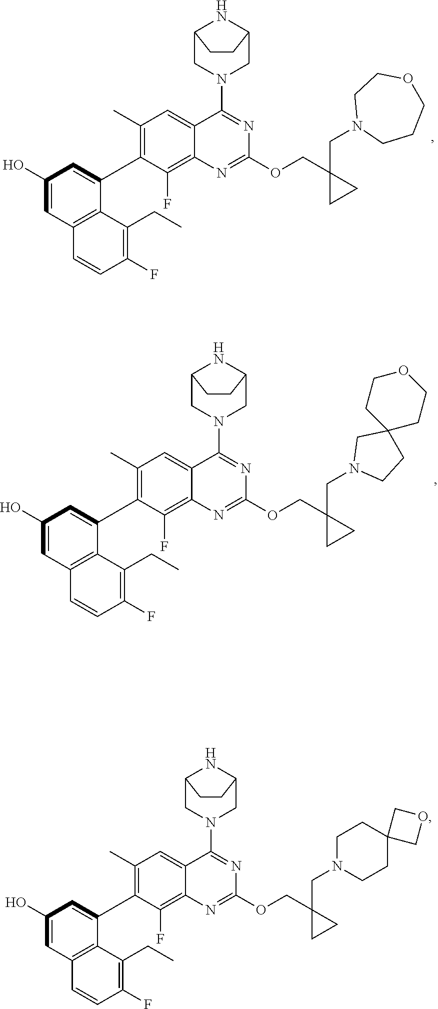

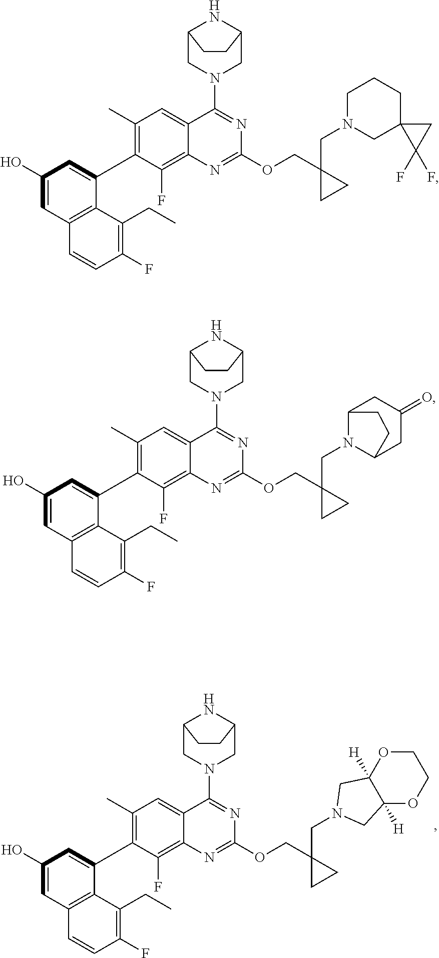

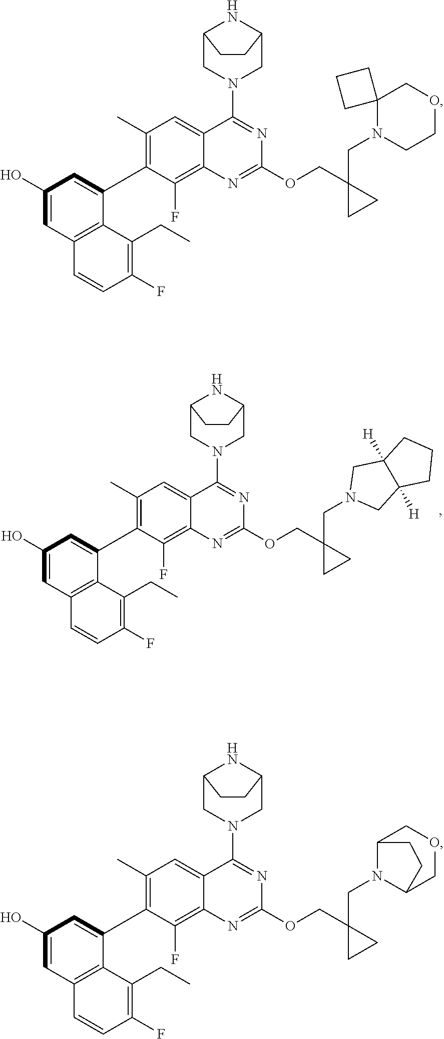

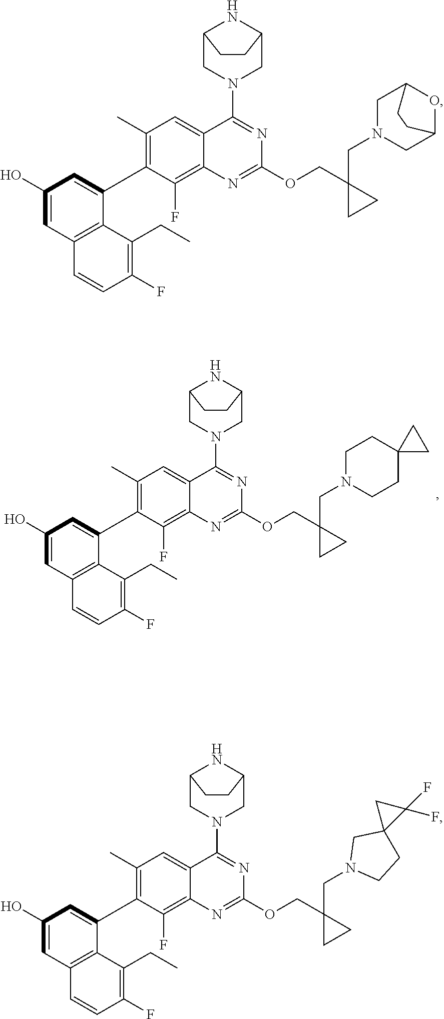

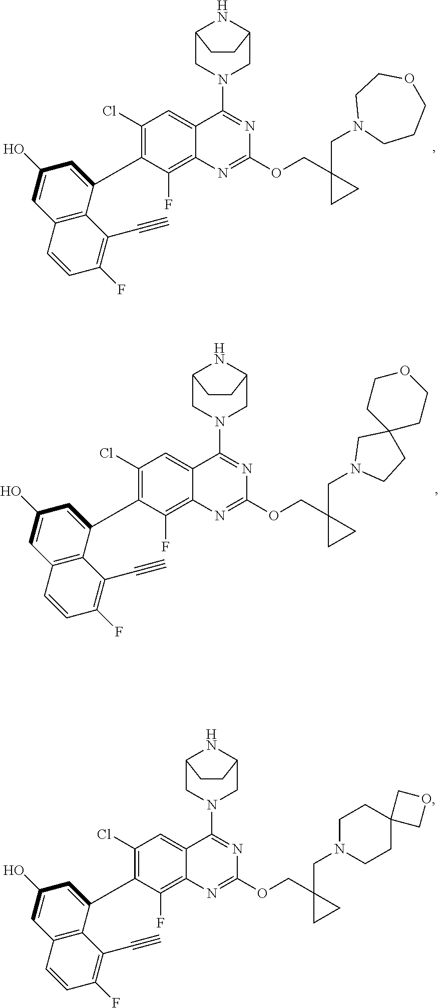

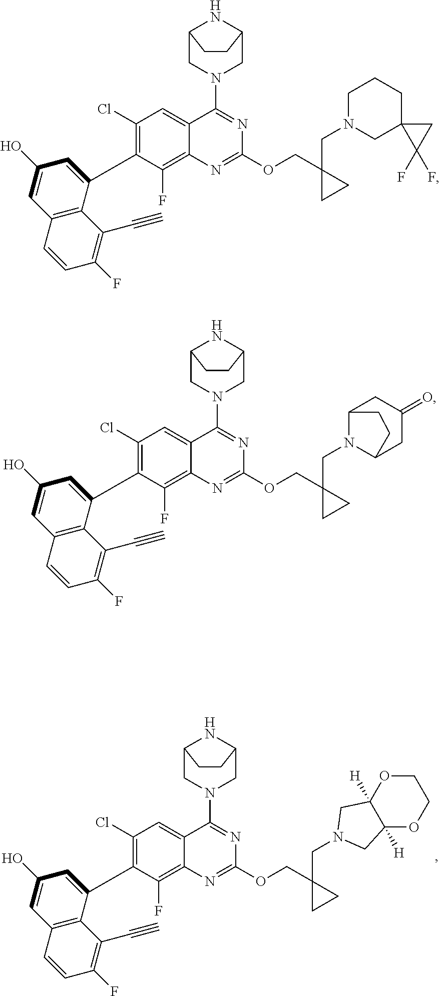

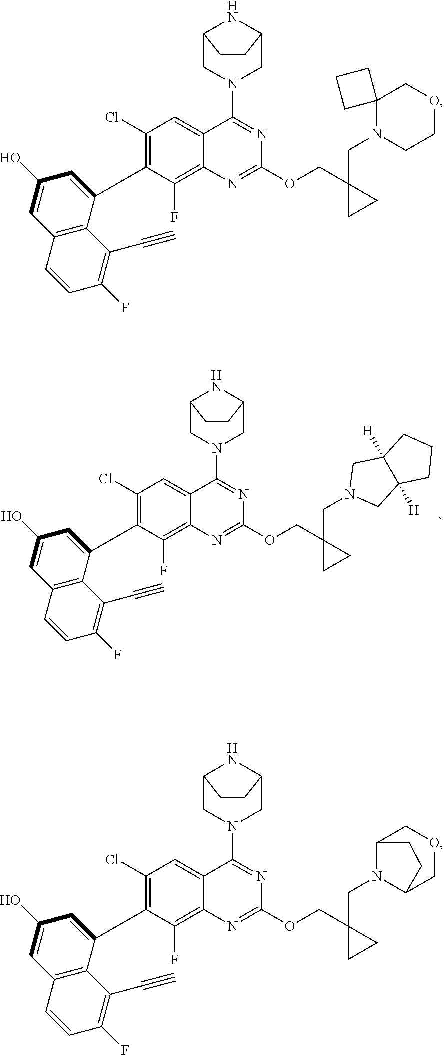

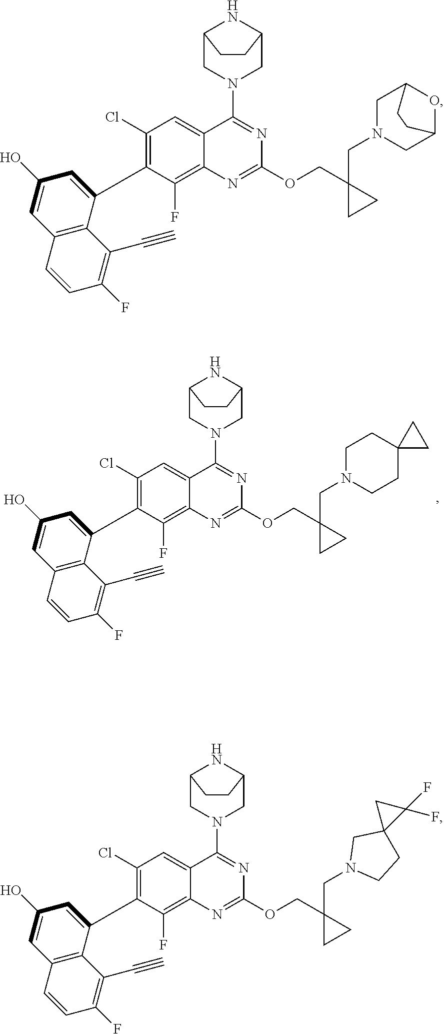

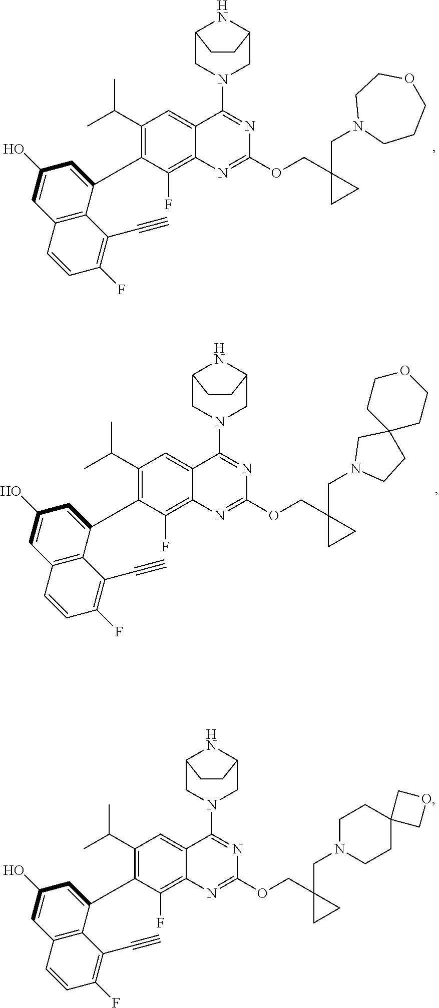

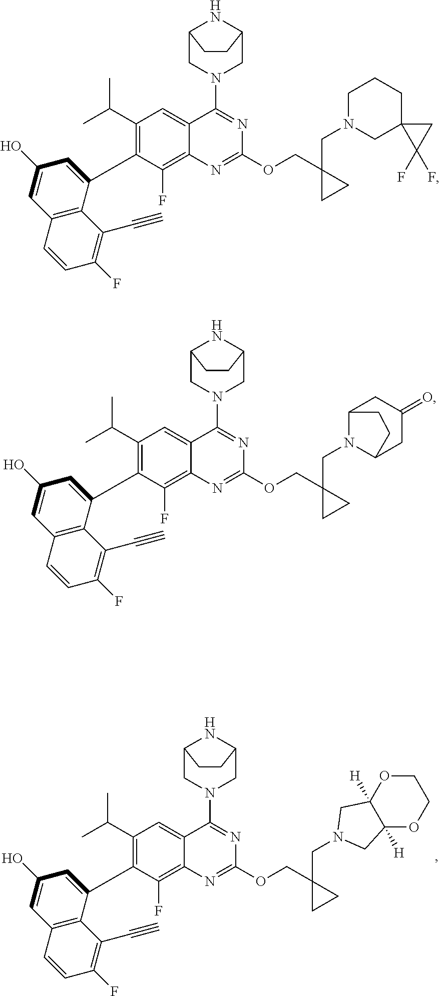

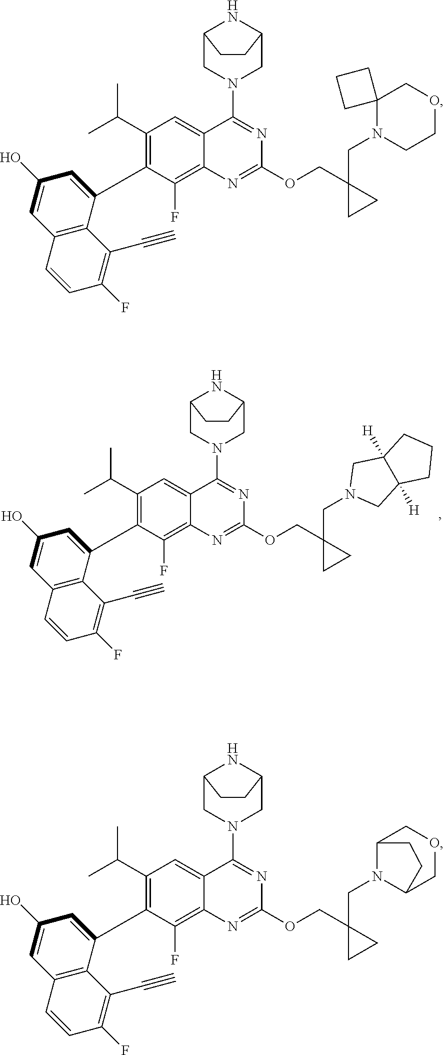

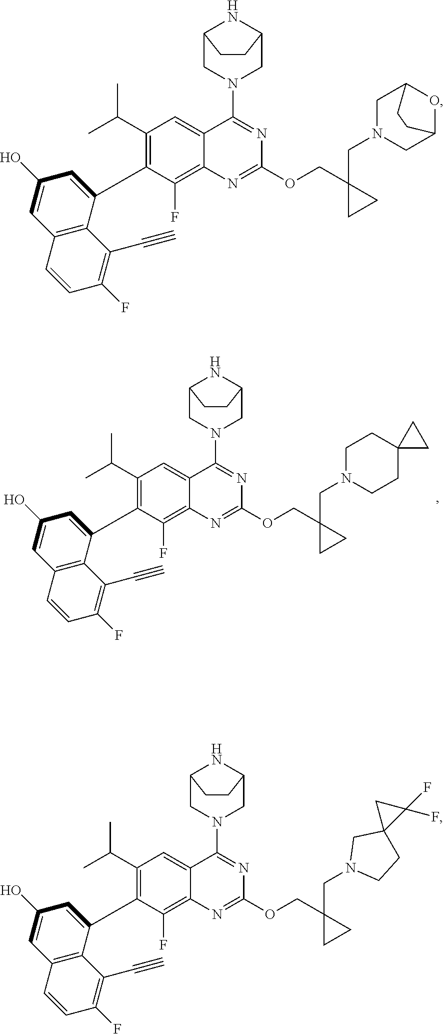

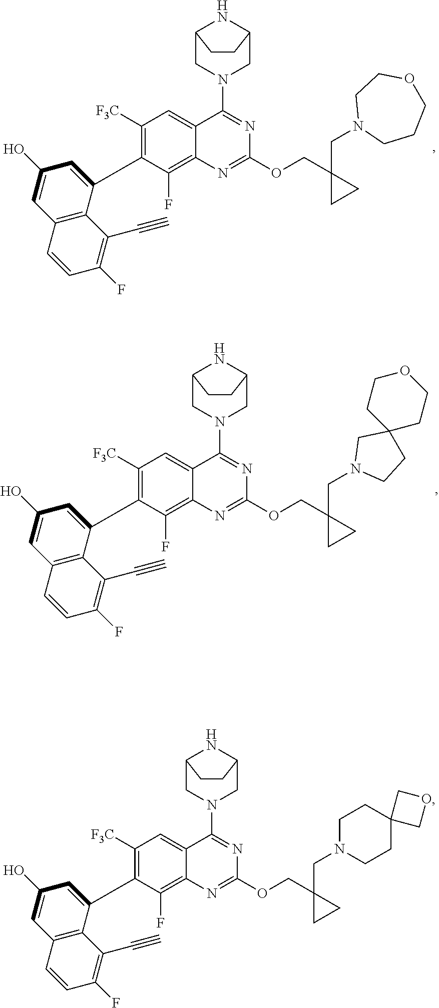

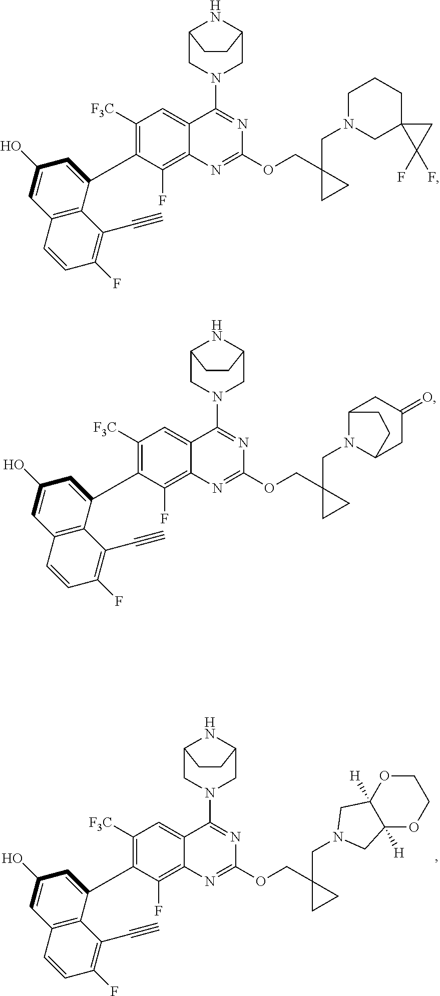

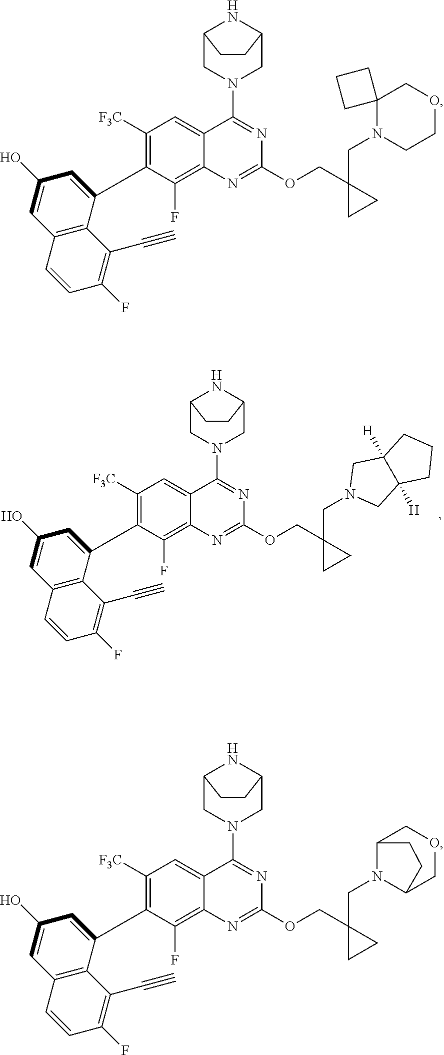

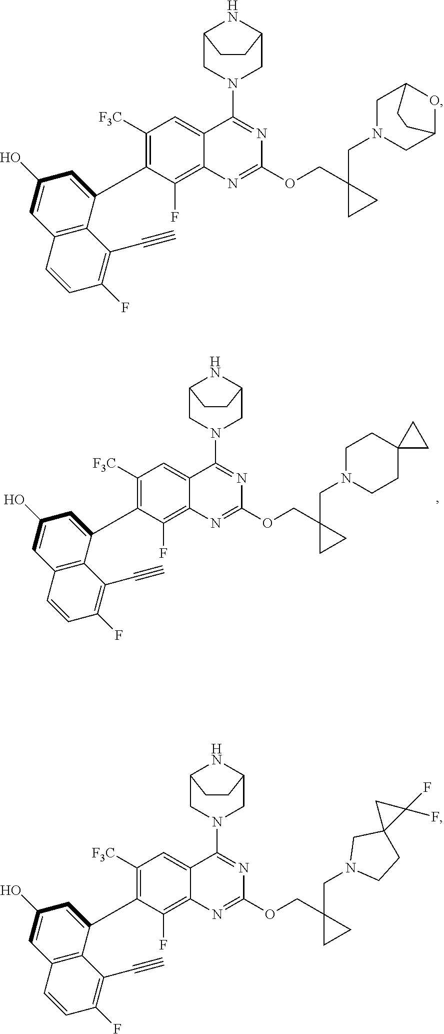

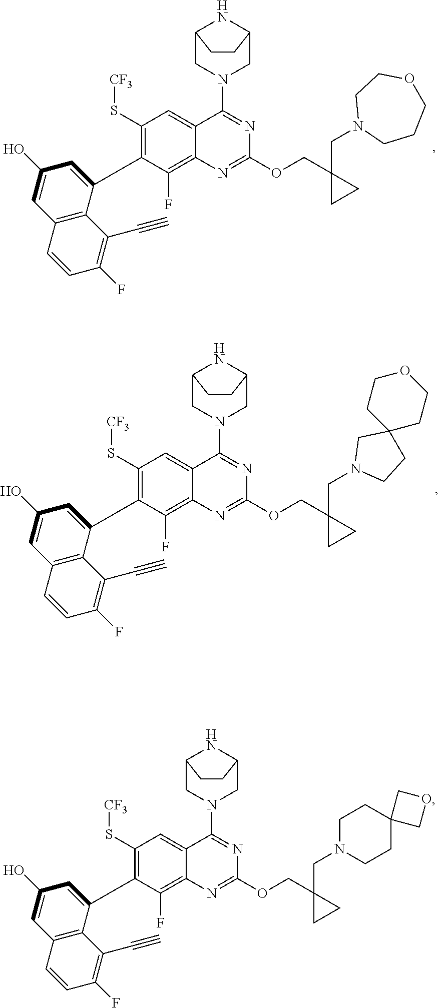

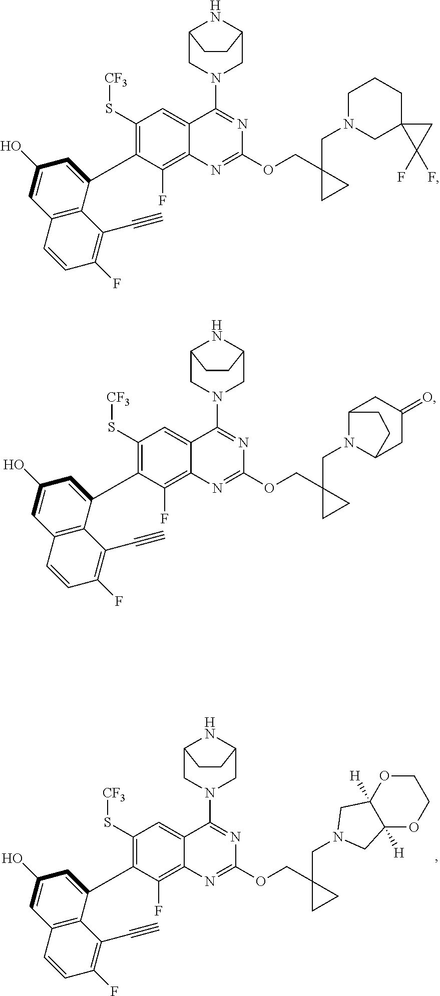

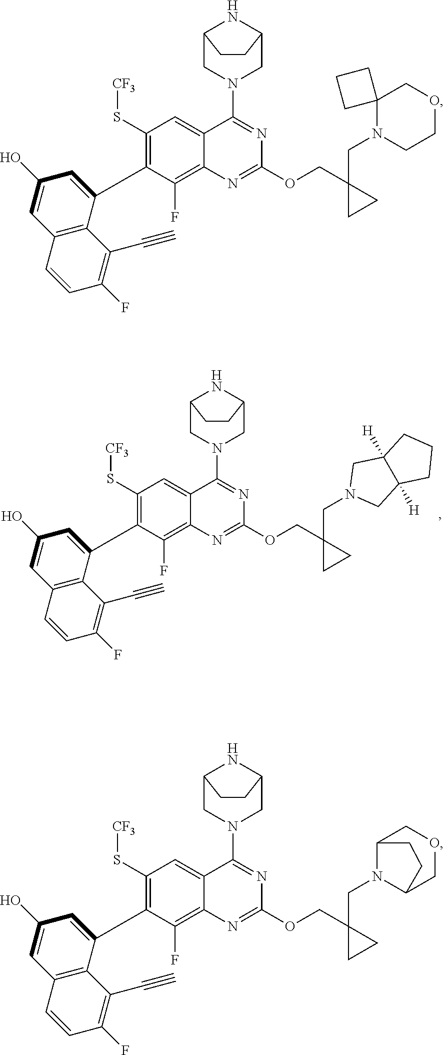

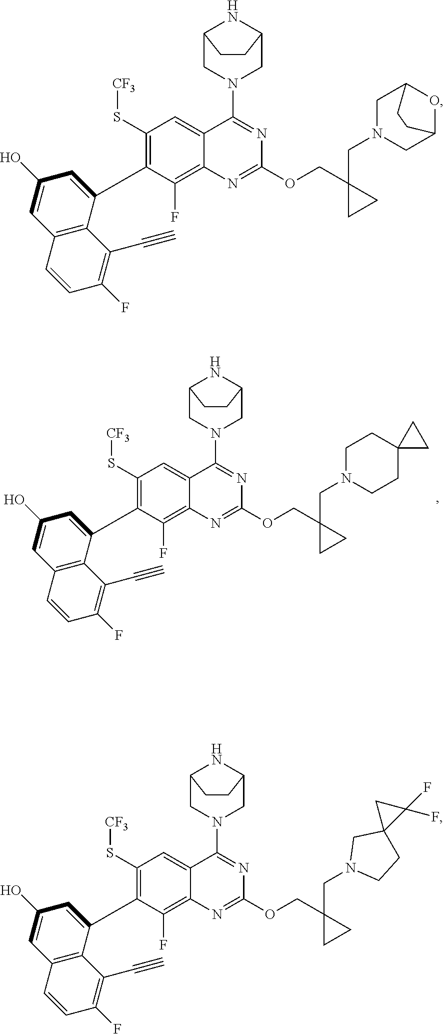

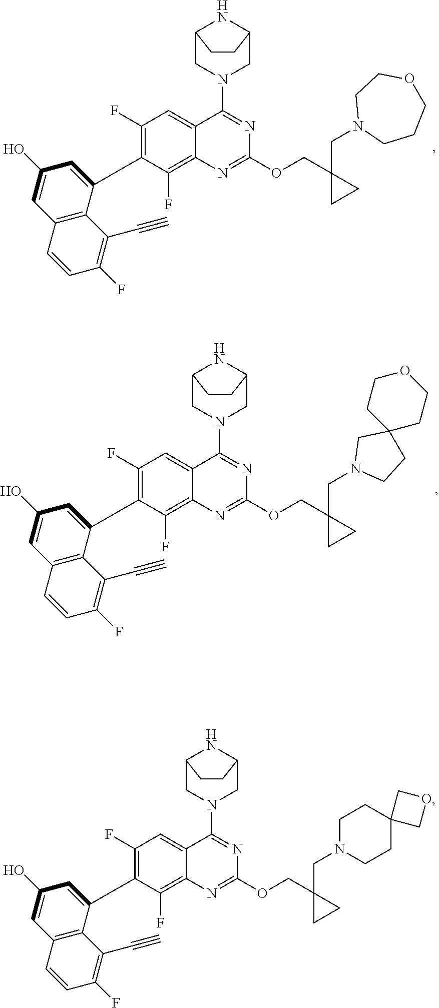

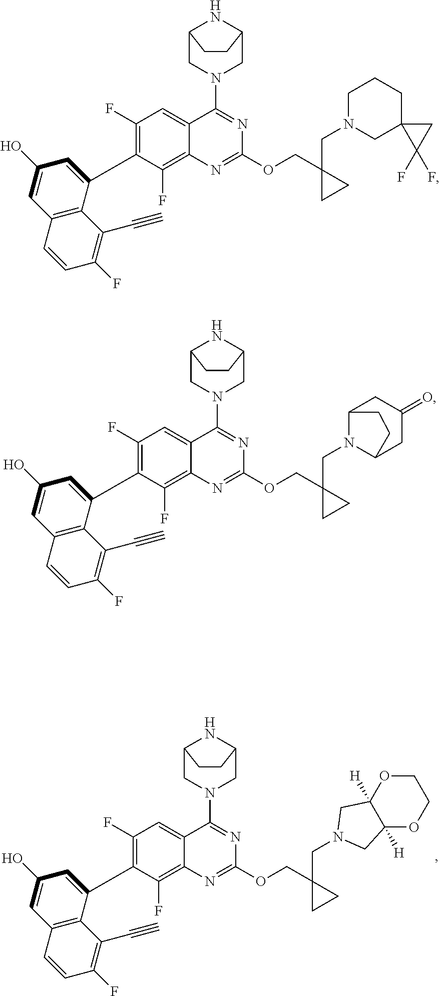

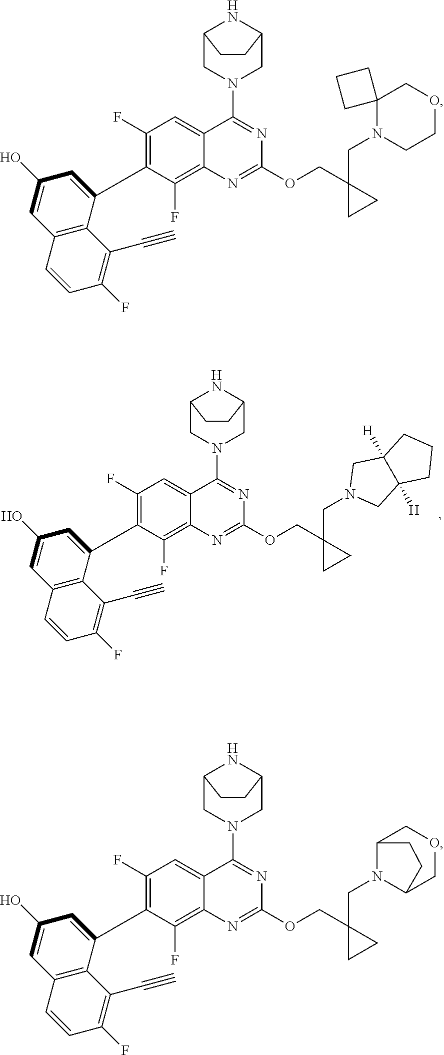

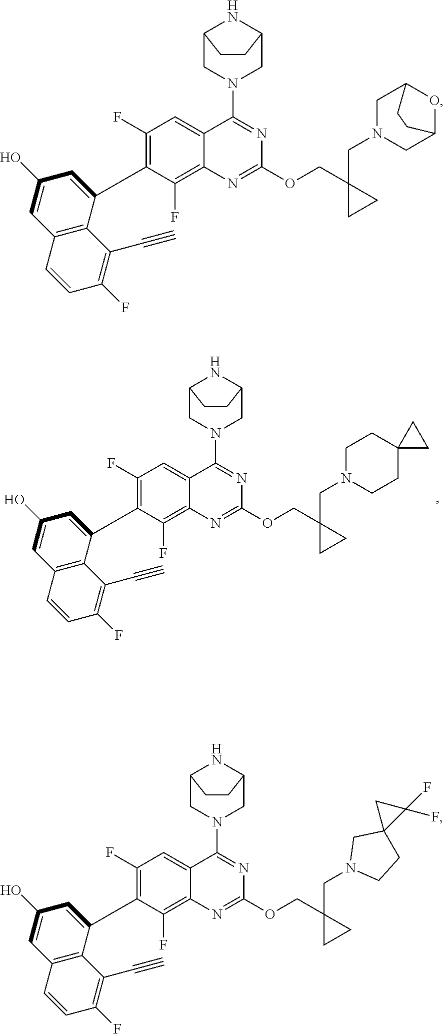

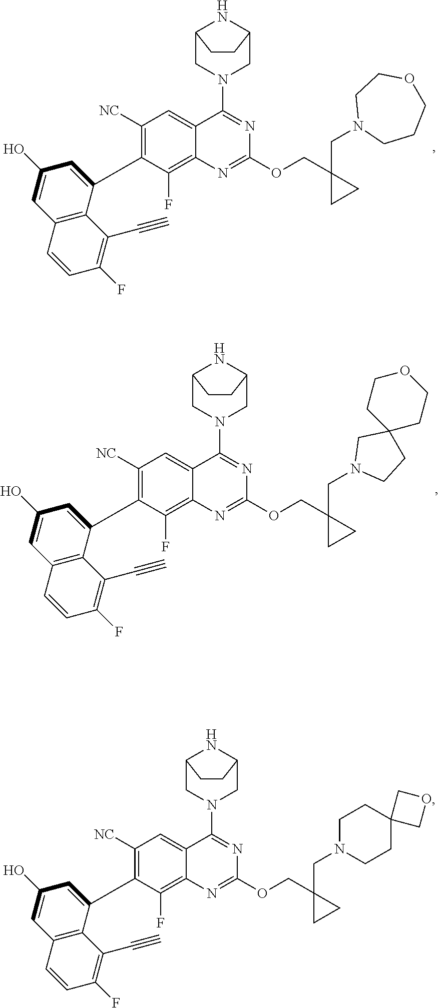

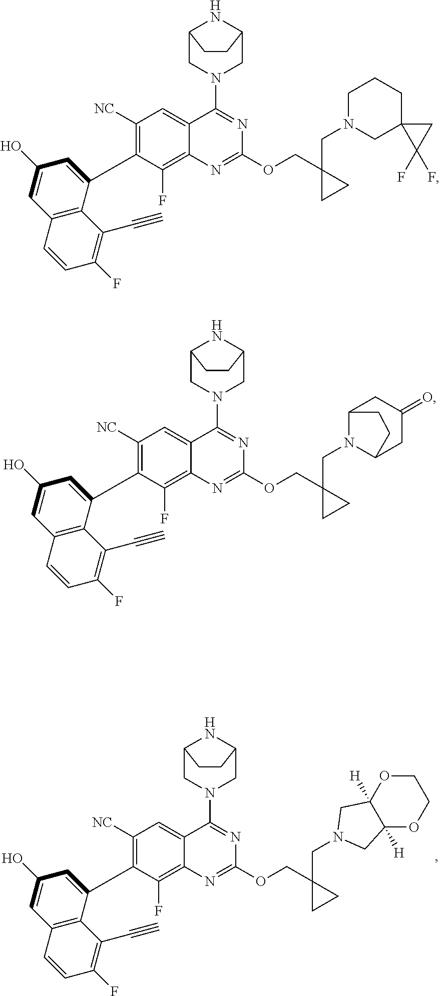

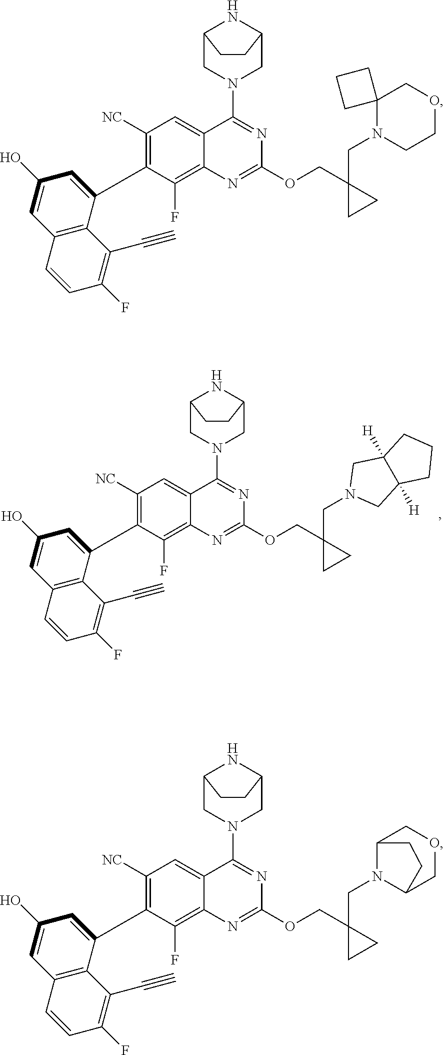

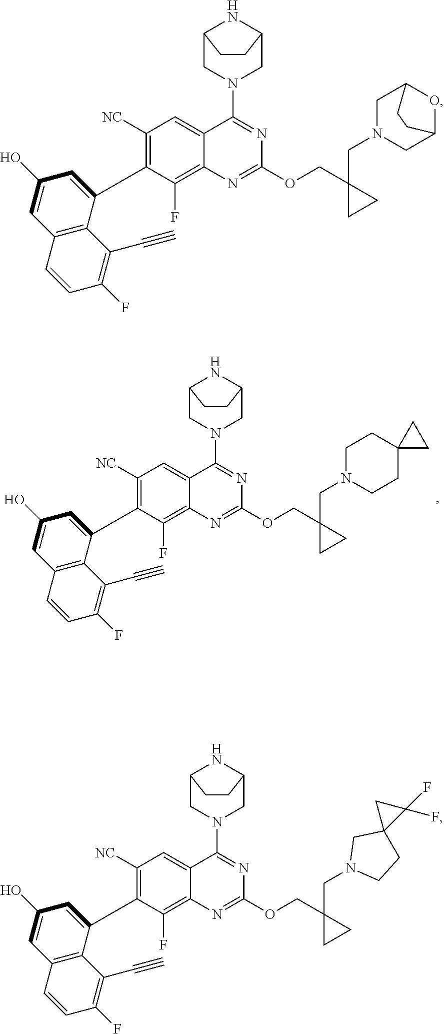

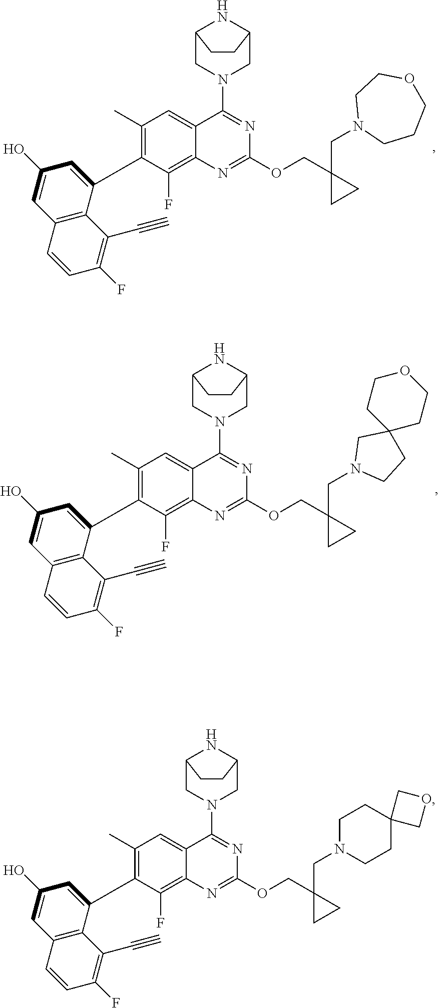

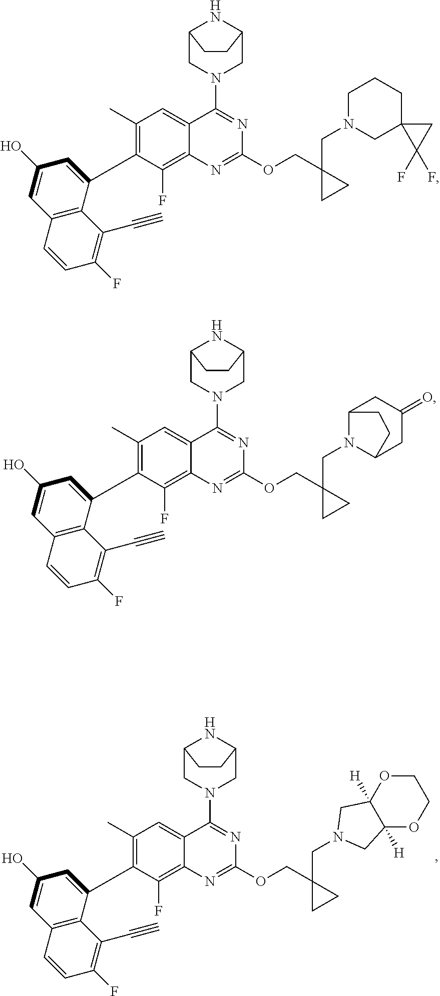

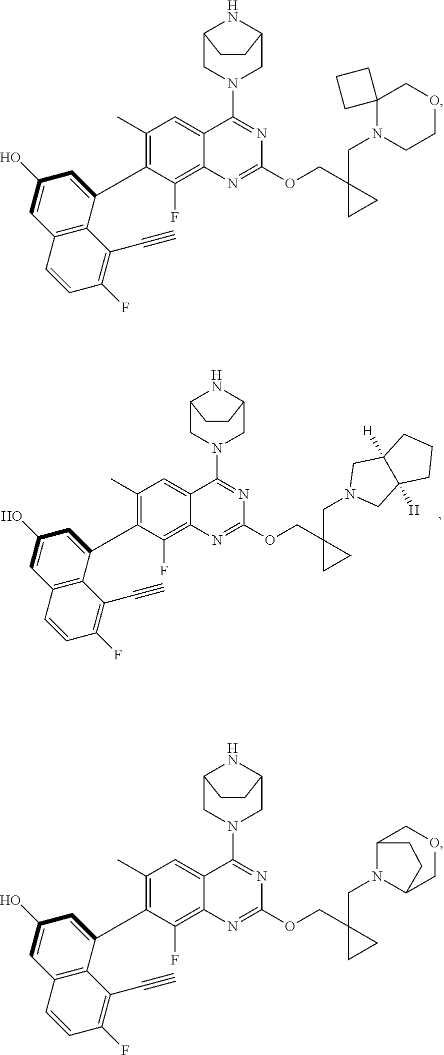

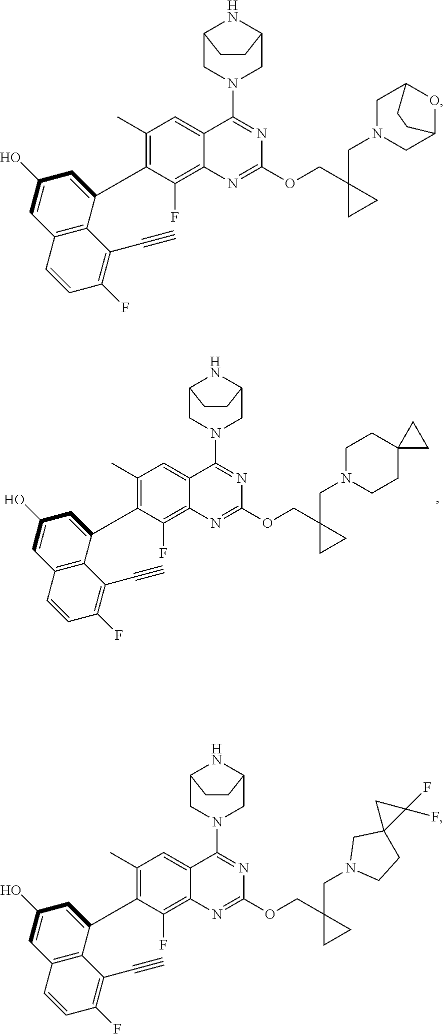

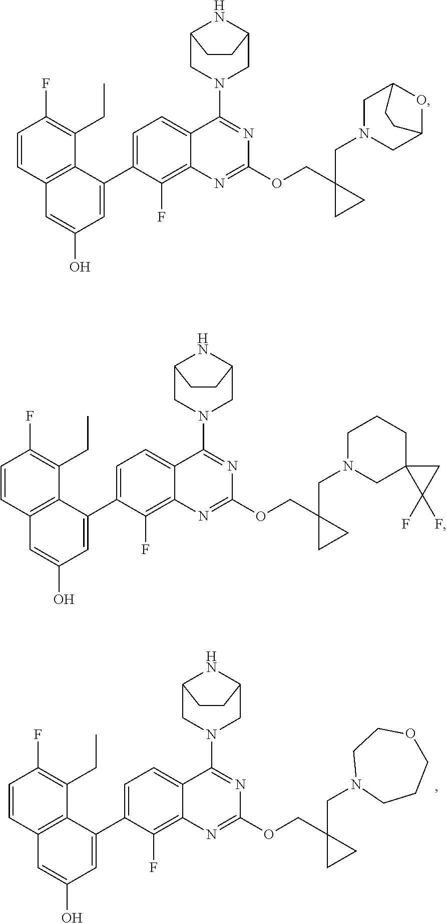 [SMILES: CCc1c(F)ccc2cc(O)cc(-c3ccc4c(N5CC6CCC(C5)N6)nc(OCC5(CN6CC7CCC(C6)O7)CC5)nc4c3F)c12 ; CCc1c(F)ccc2cc(O)cc(-c3ccc4c(N5CC6CCC(C5)N6)nc(OCC5(CN6CCCC7(C6)CC7(F)F)CC5)nc4c3F)c12 ; CCc1c(F)ccc2cc(O)cc(-c3ccc4c(N5CC6CCC(C5)N6)nc(OCC5(CN6CCCOCC6)CC5)nc4c3F)c12]

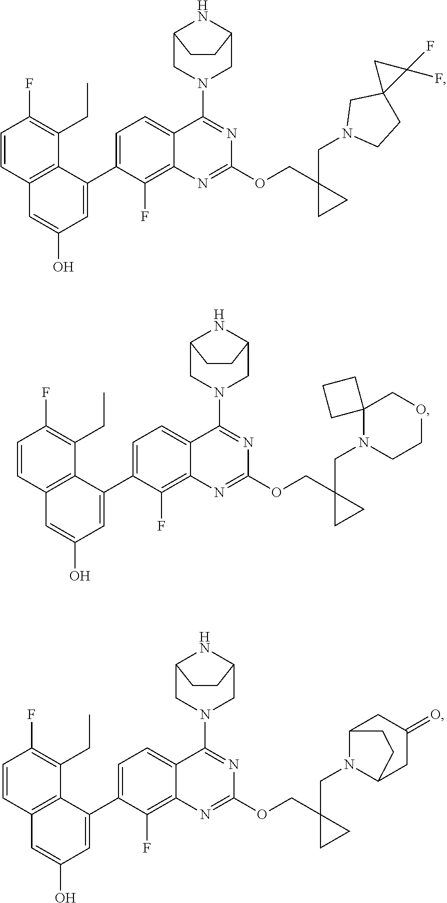 [SMILES: CCc1c(F)ccc2cc(O)cc(-c3ccc4c(N5CC6CCC(C5)N6)nc(OCC5(CN6CCC7(C6)CC7(F)F)CC5)nc4c3F)c12 ; CCc1c(F)ccc2cc(O)cc(-c3ccc4c(N5CC6CCC(C5)N6)nc(OCC5(CN6CCOCC67CCC7)CC5)nc4c3F)c12 ; CCc1c(F)ccc2cc(O)cc(-c3ccc4c(N5CC6CCC(C5)N6)nc(OCC5(CN6C7CCC6CC(=O)C7)CC5)nc4c3F)c12]

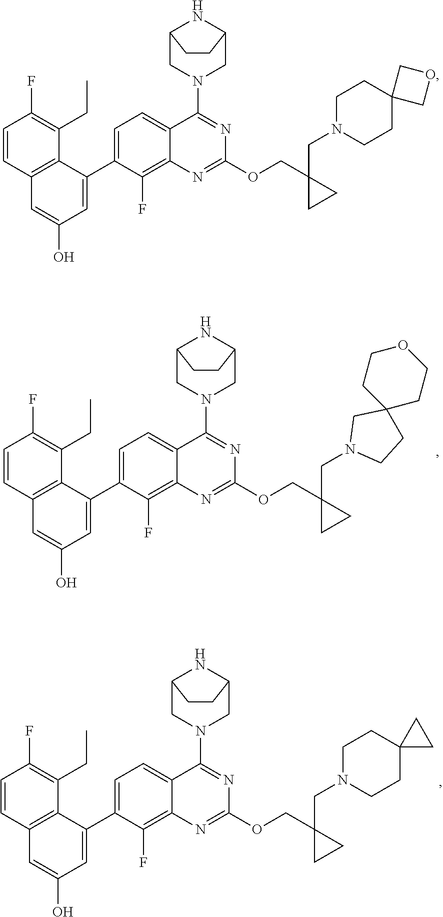 [SMILES: CCc1c(F)ccc2cc(O)cc(-c3ccc4c(N5CC6CCC(C5)N6)nc(OCC5(CN6CCC7(CC6)COC7)CC5)nc4c3F)c12 ; CCc1c(F)ccc2cc(O)cc(-c3ccc4c(N5CC6CCC(C5)N6)nc(OCC5(CN6CCC7(CCOCC7)C6)CC5)nc4c3F)c12 ; CCc1c(F)ccc2cc(O)cc(-c3ccc4c(N5CC6CCC(C5)N6)nc(OCC5(CN6CCC7(CC6)CC7)CC5)nc4c3F)c12]

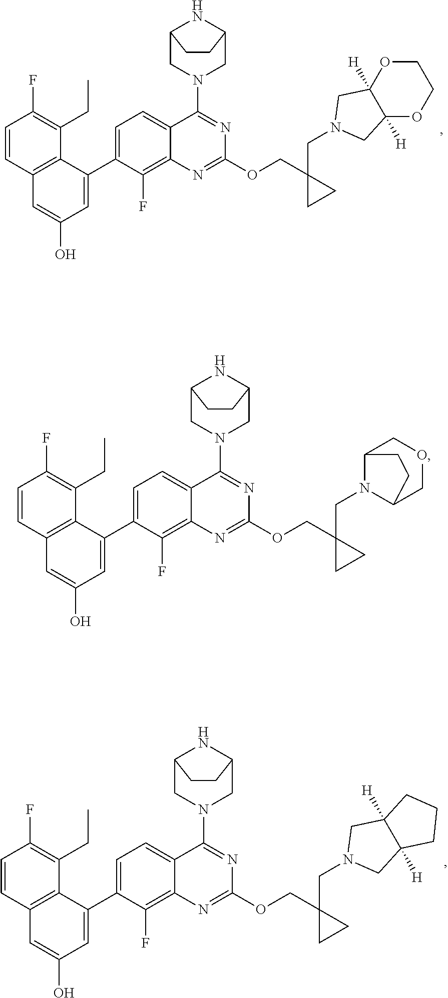 [SMILES: CCc1c(F)ccc2cc(O)cc(-c3ccc4c(N5CC6CCC(C5)N6)nc(OCC5(CN6C[C@@H]7OCCO[C@@H]7C6)CC5)nc4c3F)c12 ; CCc1c(F)ccc2cc(O)cc(-c3ccc4c(N5CC6CCC(C5)N6)nc(OCC5(CN6C7CCC6COC7)CC5)nc4c3F)c12 ; CCc1c(F)ccc2cc(O)cc(-c3ccc4c(N5CC6CCC(C5)N6)nc(OCC5(CN6C[C@H]7CCC[C@H]7C6)CC5)nc4c3F)c12]

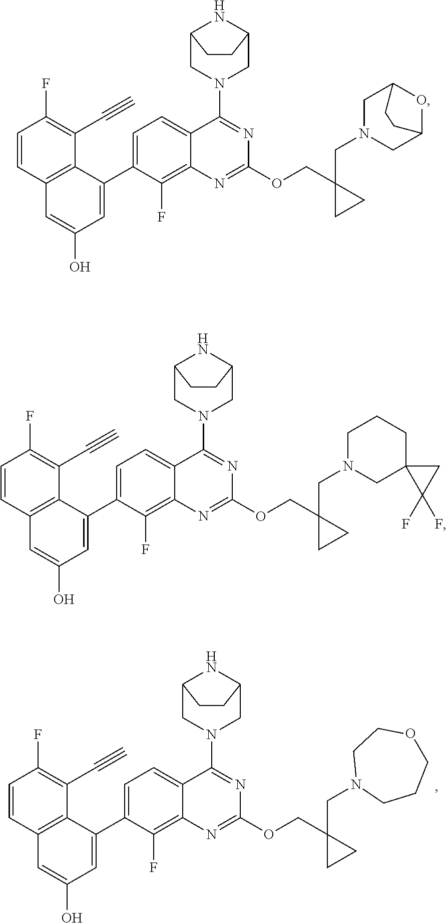 [SMILES: C#Cc1c(F)ccc2cc(O)cc(-c3ccc4c(N5CC6CCC(C5)N6)nc(OCC5(CN6CC7CCC(C6)O7)CC5)nc4c3F)c12 ; C#Cc1c(F)ccc2cc(O)cc(-c3ccc4c(N5CC6CCC(C5)N6)nc(OCC5(CN6CCCC7(C6)CC7(F)F)CC5)nc4c3F)c12 ; C#Cc1c(F)ccc2cc(O)cc(-c3ccc4c(N5CC6CCC(C5)N6)nc(OCC5(CN6CCCOCC6)CC5)nc4c3F)c12]

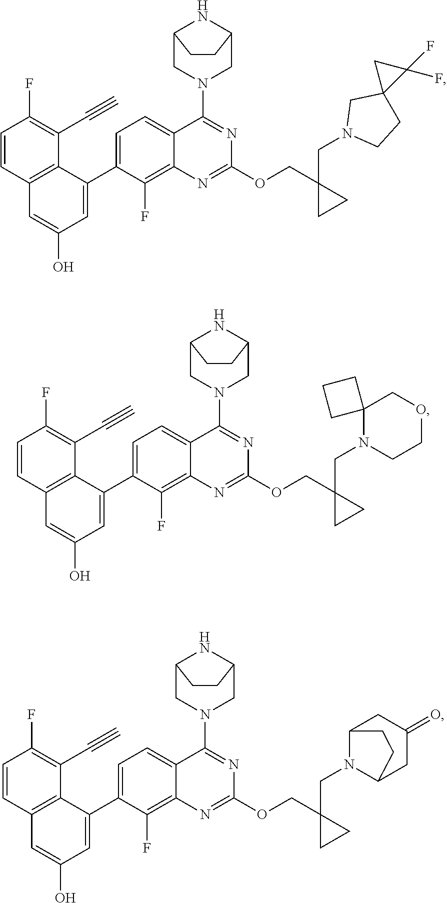 [SMILES: C#Cc1c(F)ccc2cc(O)cc(-c3ccc4c(N5CC6CCC(C5)N6)nc(OCC5(CN6CCC7(C6)CC7(F)F)CC5)nc4c3F)c12 ; C#Cc1c(F)ccc2cc(O)cc(-c3ccc4c(N5CC6CCC(C5)N6)nc(OCC5(CN6CCOCC67CCC7)CC5)nc4c3F)c12 ; C#Cc1c(F)ccc2cc(O)cc(-c3ccc4c(N5CC6CCC(C5)N6)nc(OCC5(CN6C7CCC6CC(=O)C7)CC5)nc4c3F)c12]

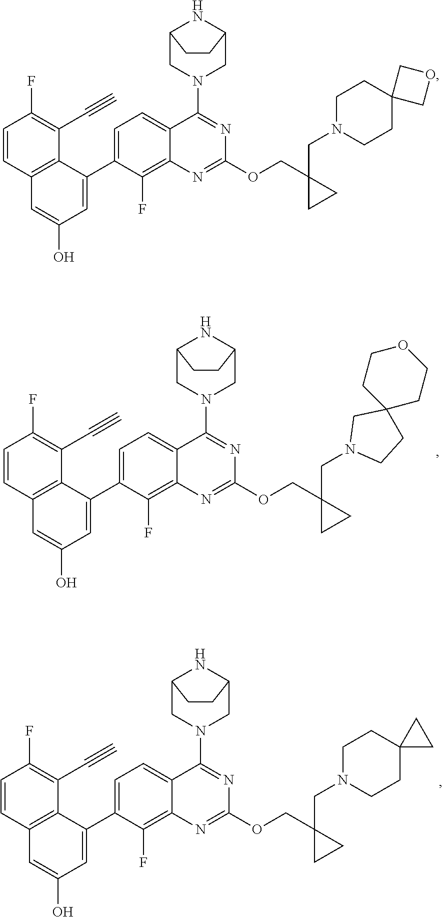 [SMILES: C#Cc1c(F)ccc2cc(O)cc(-c3ccc4c(N5CC6CCC(C5)N6)nc(OCC5(CN6CCC7(CC6)COC7)CC5)nc4c3F)c12 ; C#Cc1c(F)ccc2cc(O)cc(-c3ccc4c(N5CC6CCC(C5)N6)nc(OCC5(CN6CCC7(CCOCC7)C6)CC5)nc4c3F)c12 ; C#Cc1c(F)ccc2cc(O)cc(-c3ccc4c(N5CC6CCC(C5)N6)nc(OCC5(CN6CCC7(CC6)CC7)CC5)nc4c3F)c12]

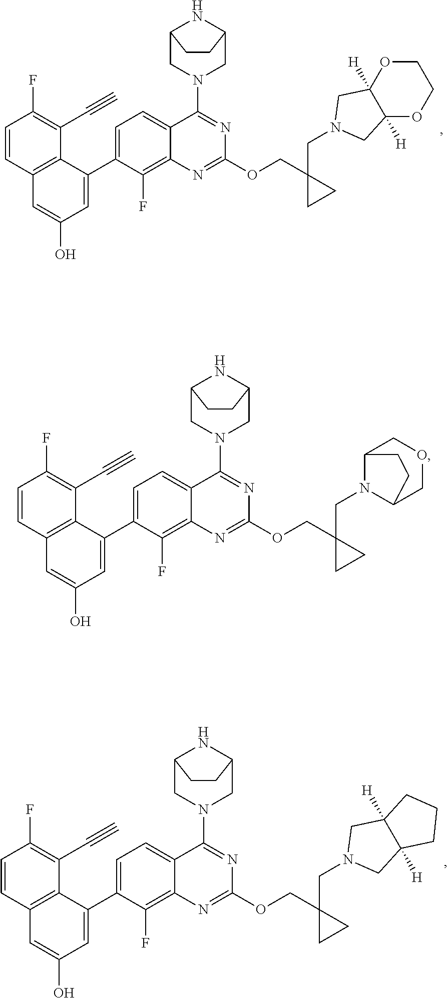 [SMILES: C#Cc1c(F)ccc2cc(O)cc(-c3ccc4c(N5CC6CCC(C5)N6)nc(OCC5(CN6C[C@@H]7OCCO[C@@H]7C6)CC5)nc4c3F)c12 ; C#Cc1c(F)ccc2cc(O)cc(-c3ccc4c(N5CC6CCC(C5)N6)nc(OCC5(CN6C7CCC6COC7)CC5)nc4c3F)c12 ; C#Cc1c(F)ccc2cc(O)cc(-c3ccc4c(N5CC6CCC(C5)N6)nc(OCC5(CN6C[C@H]7CCC[C@H]7C6)CC5)nc4c3F)c12]

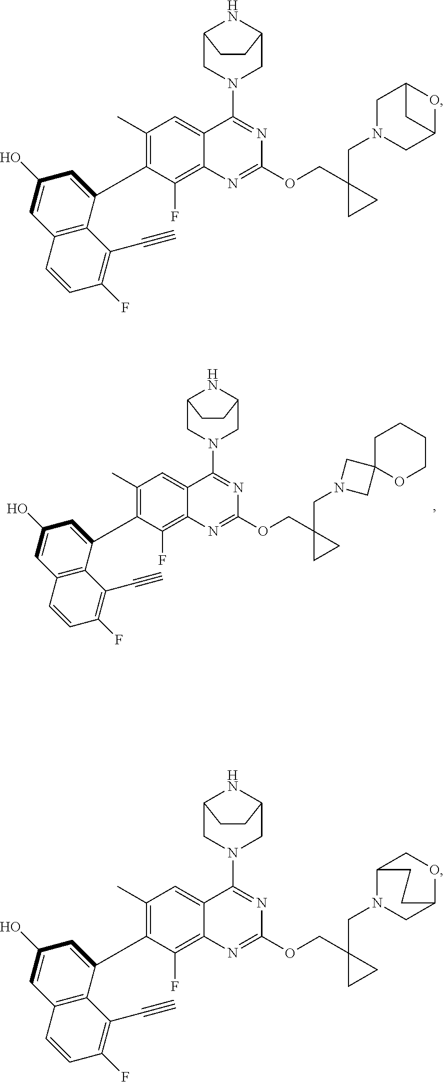

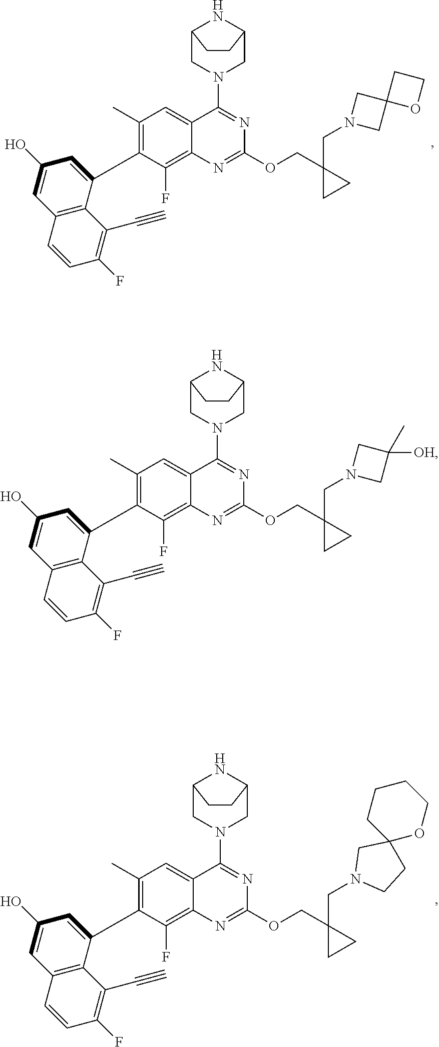

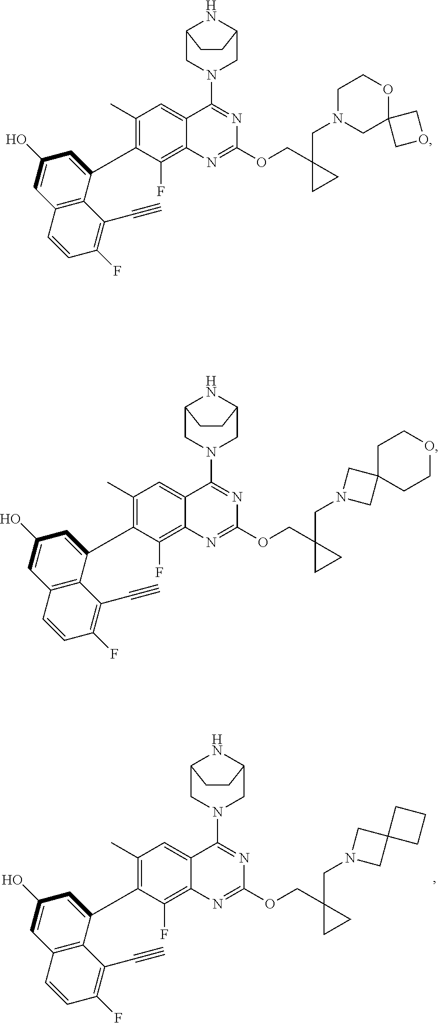

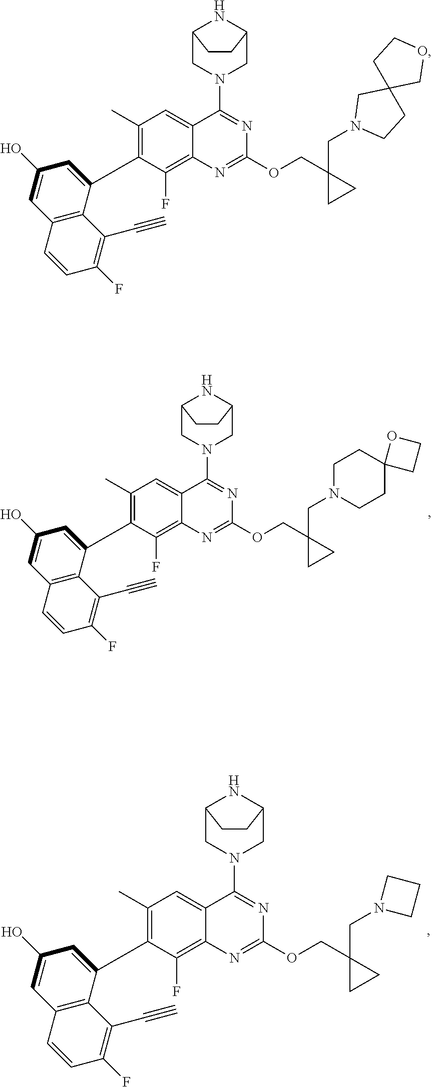

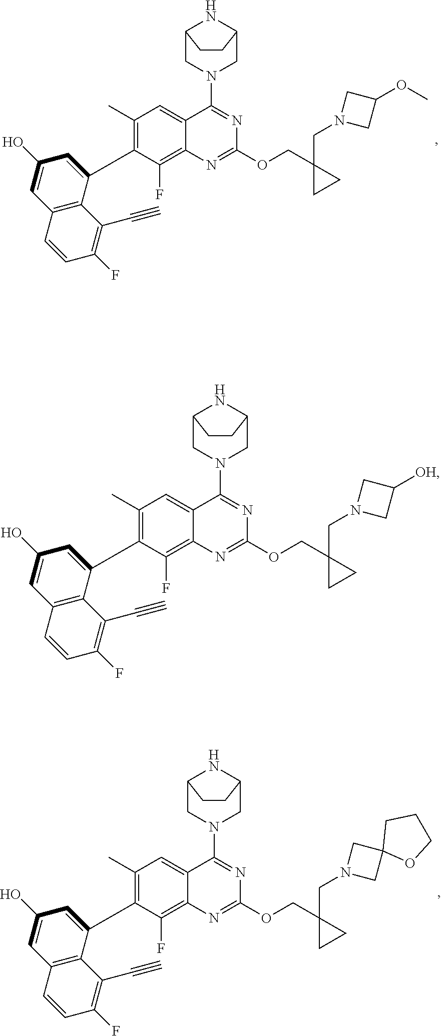

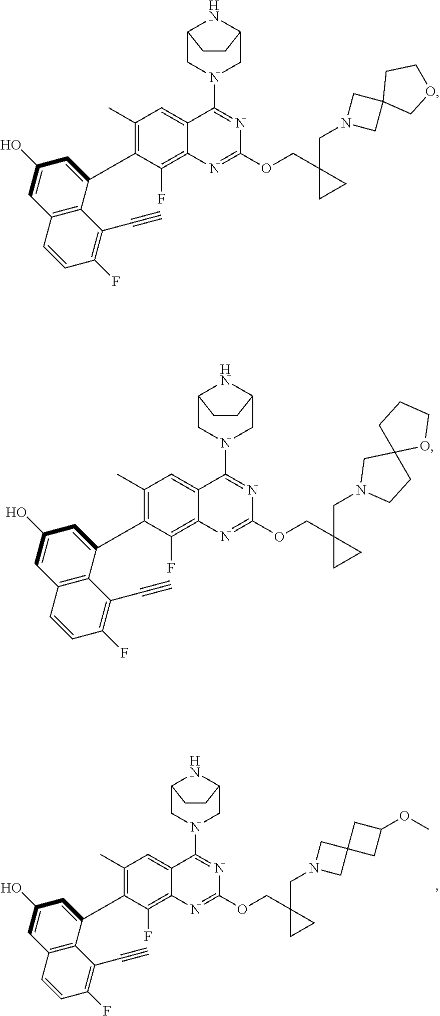

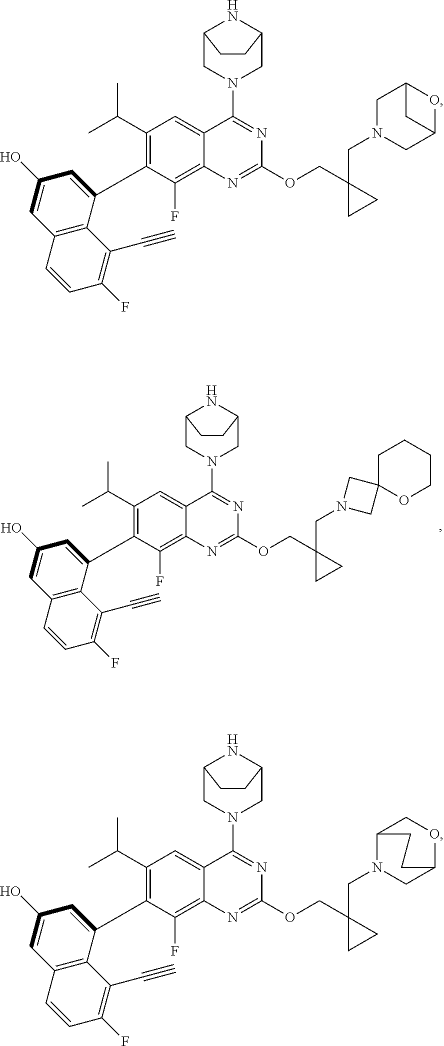

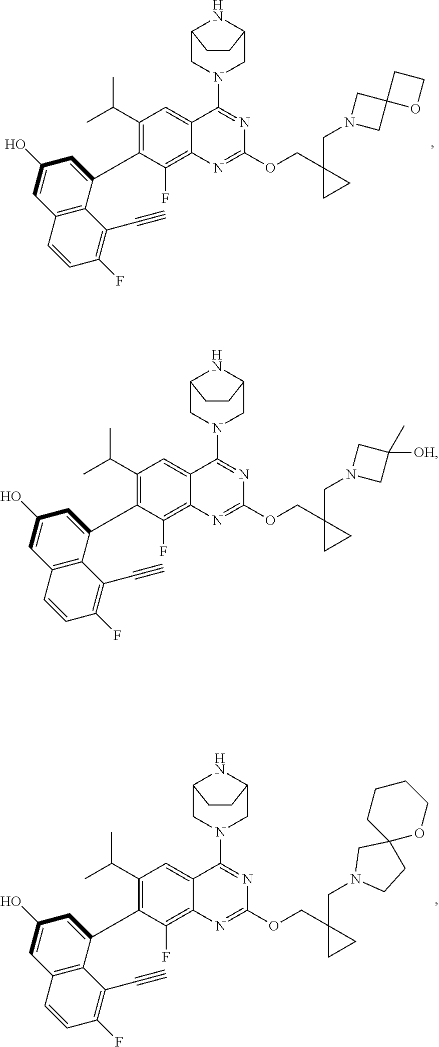

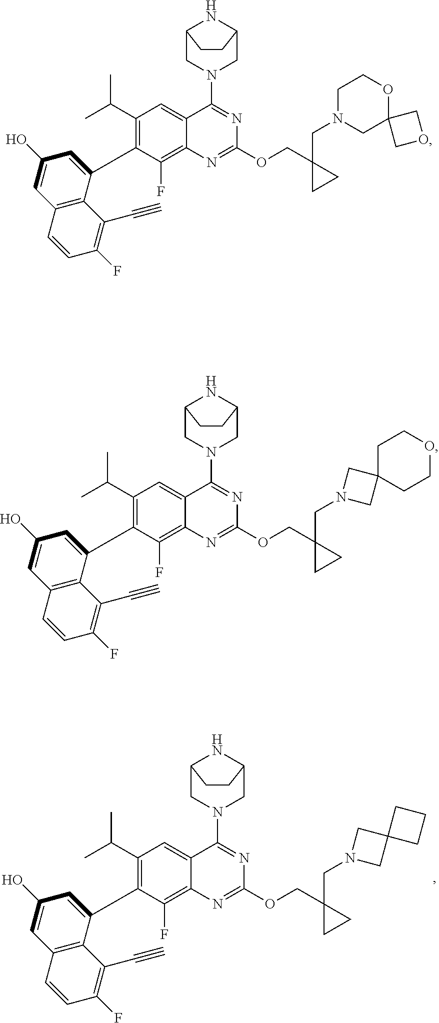

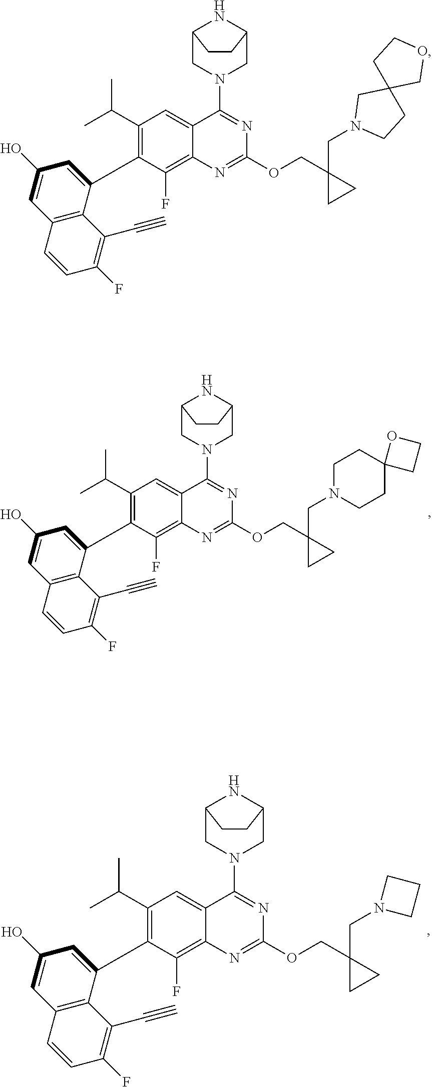

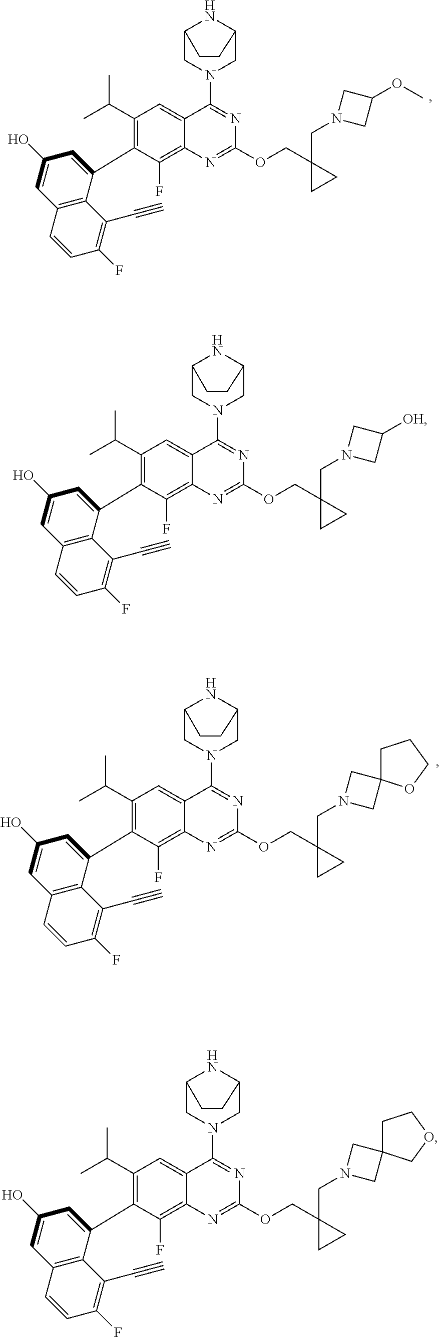

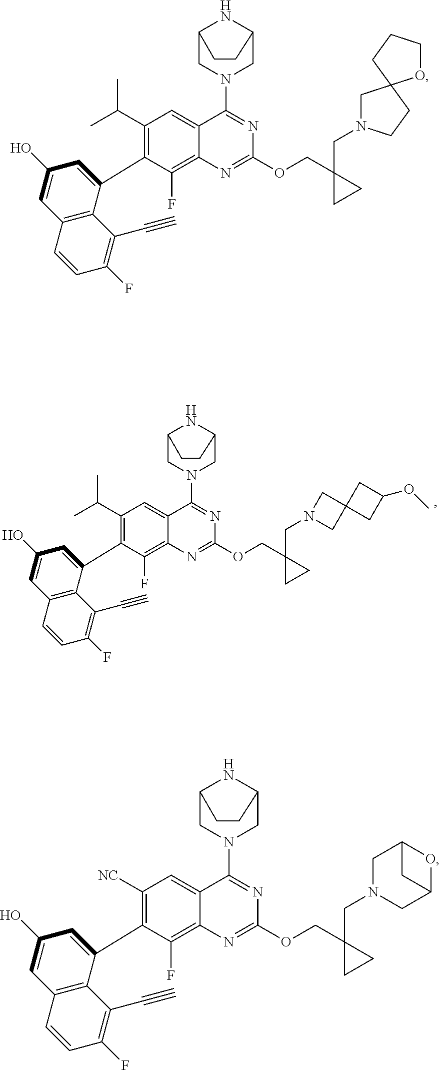

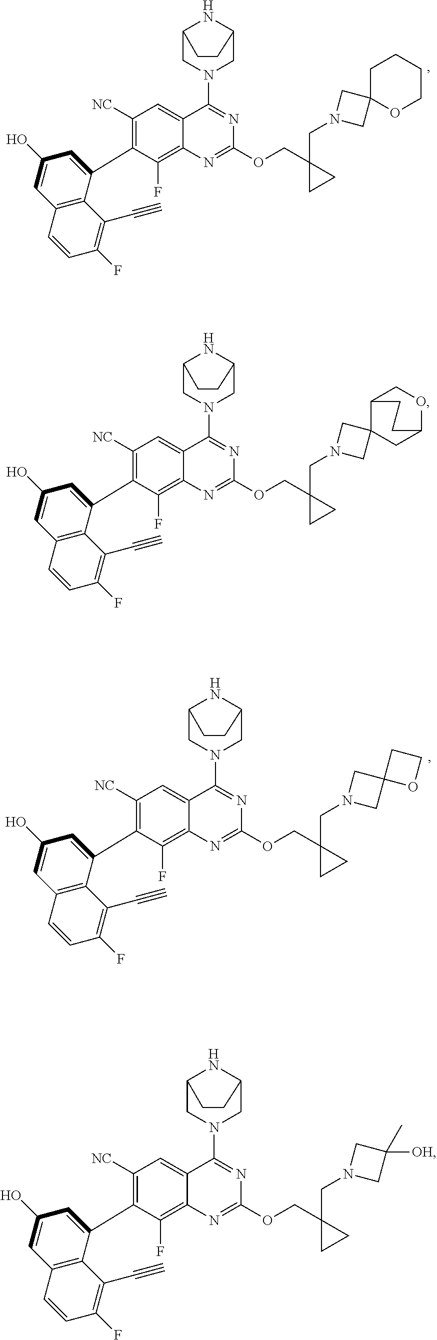

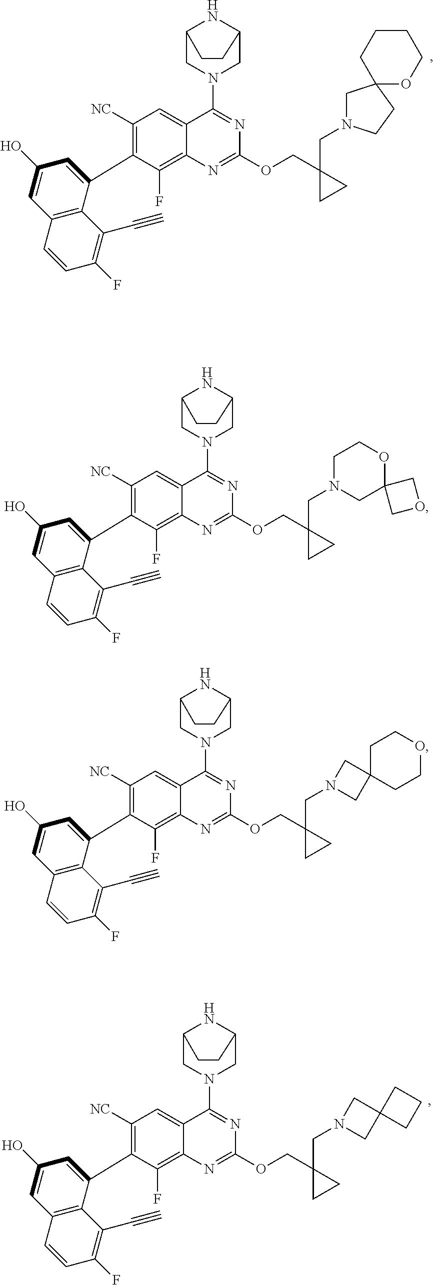

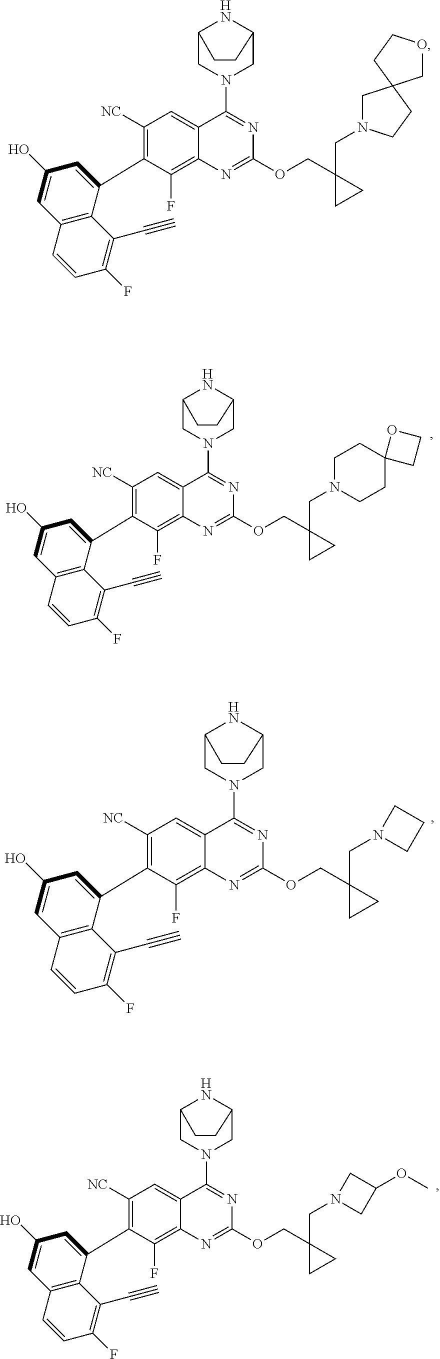

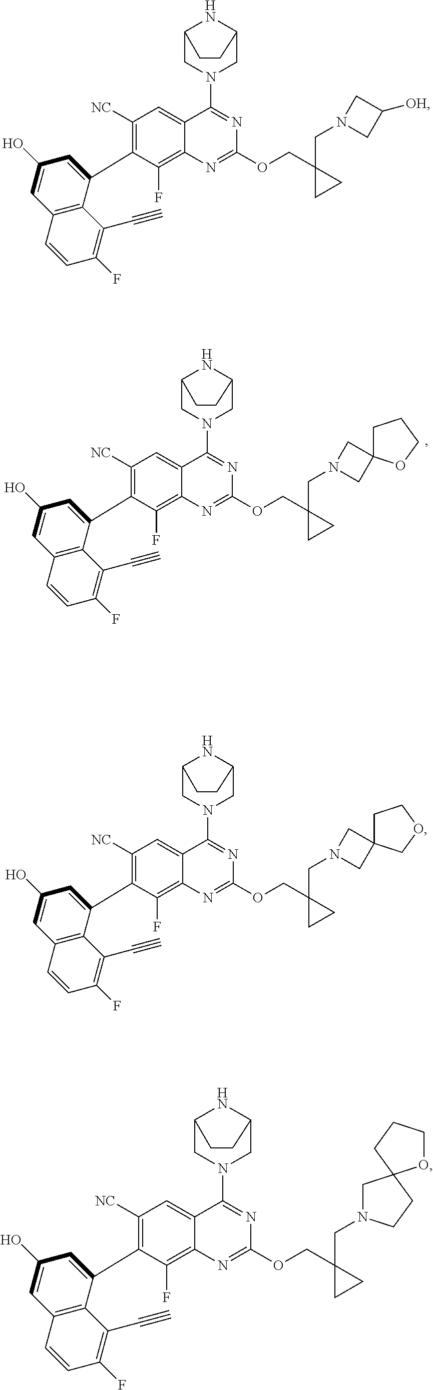

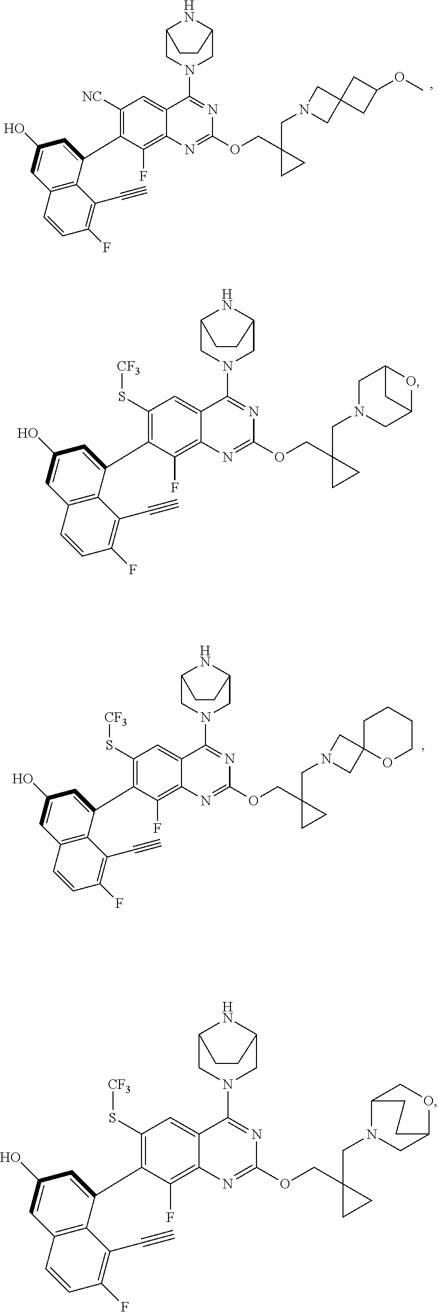

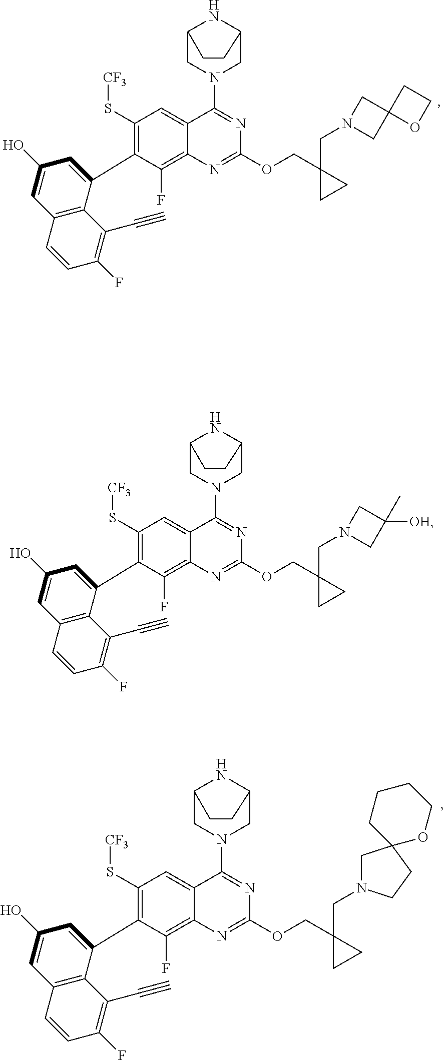

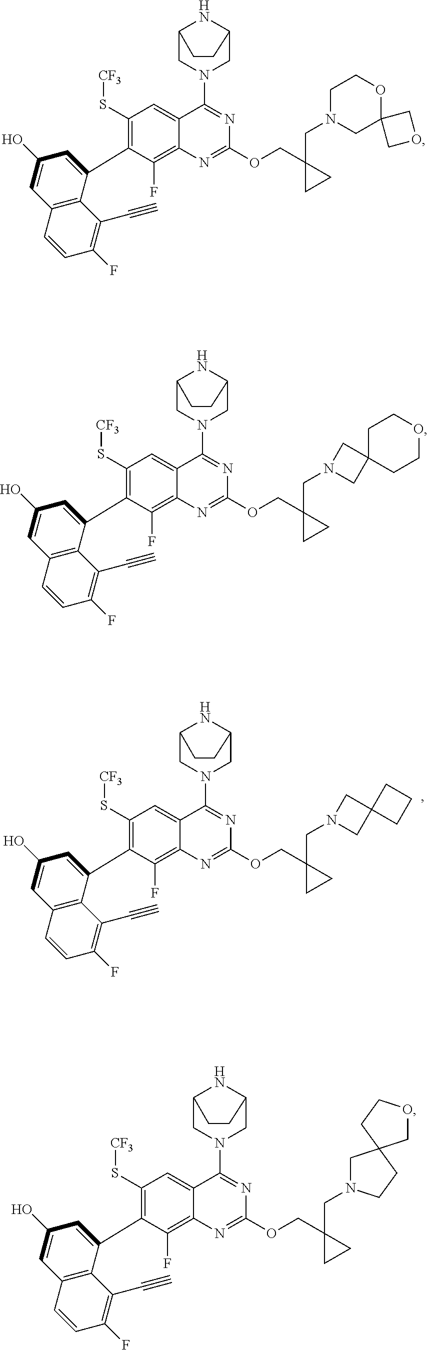

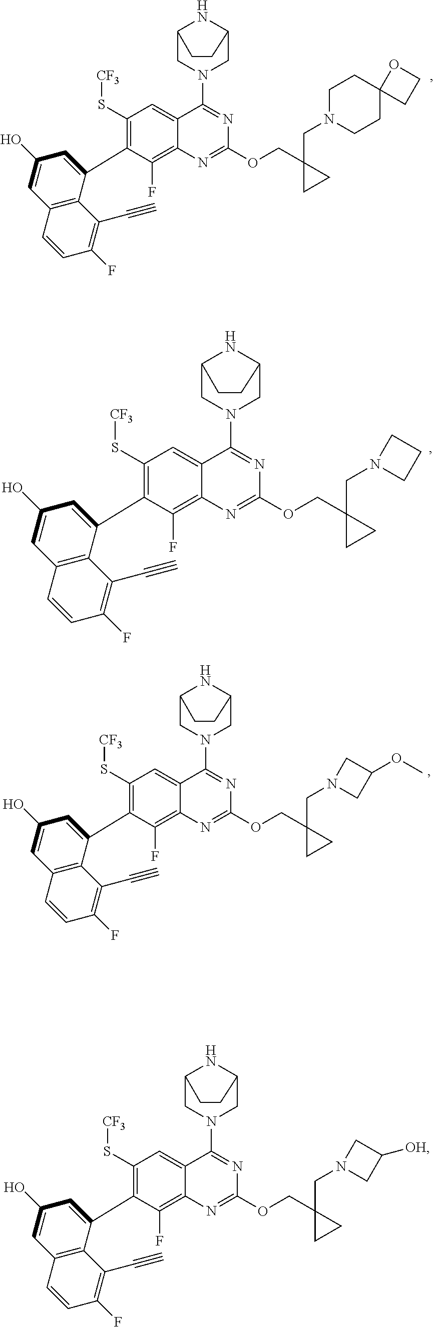

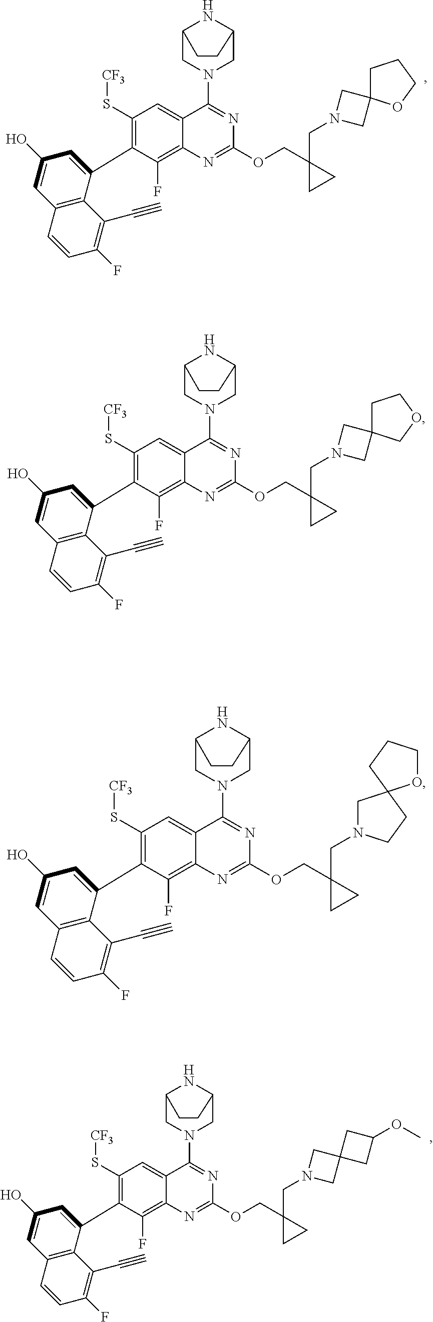

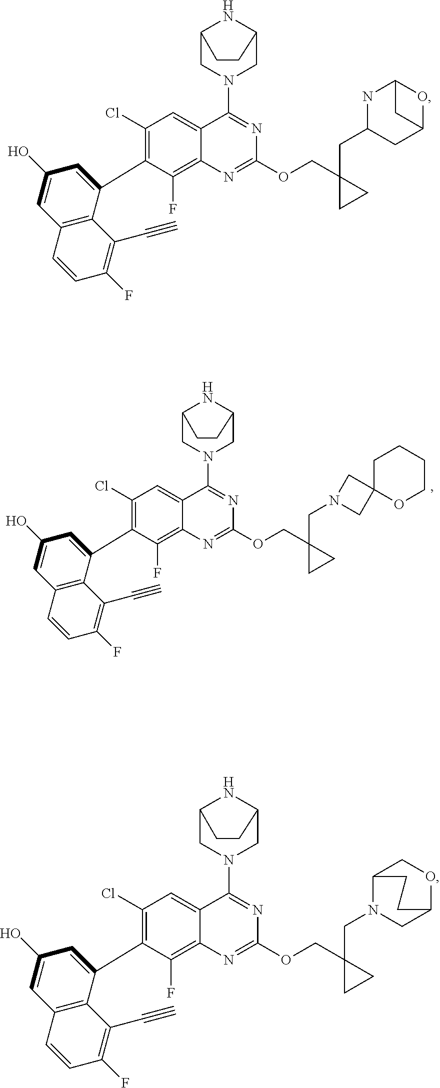

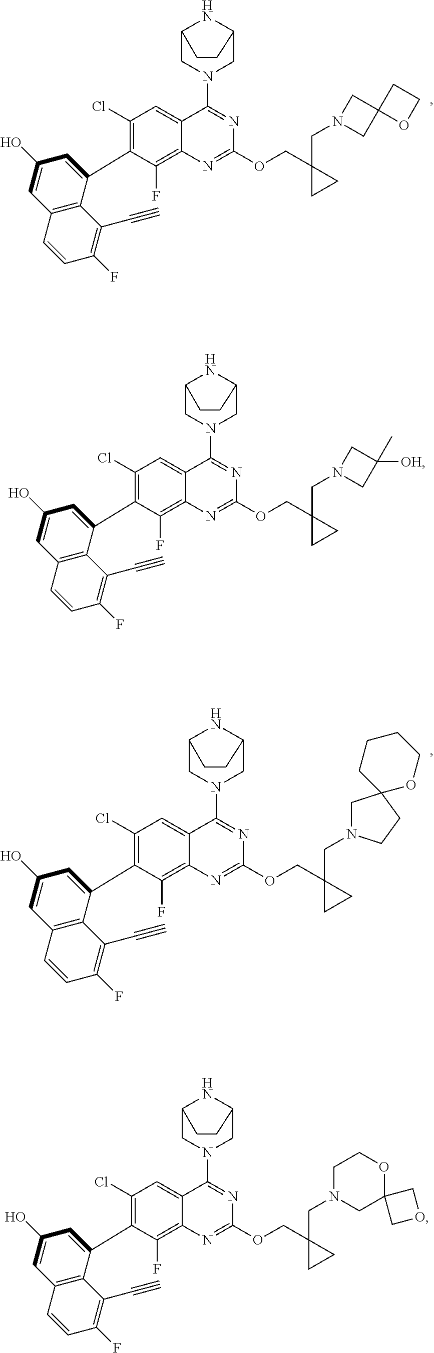

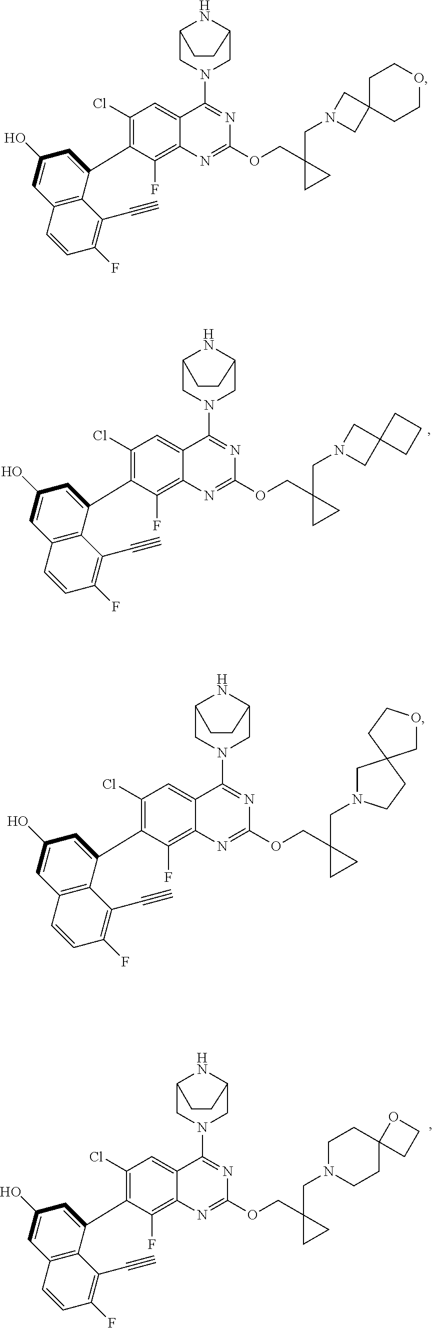

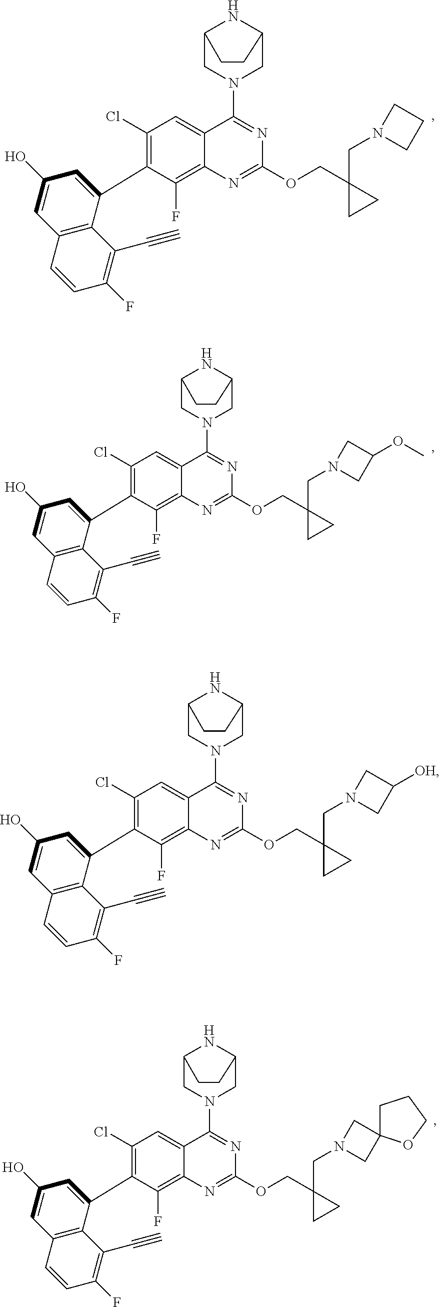

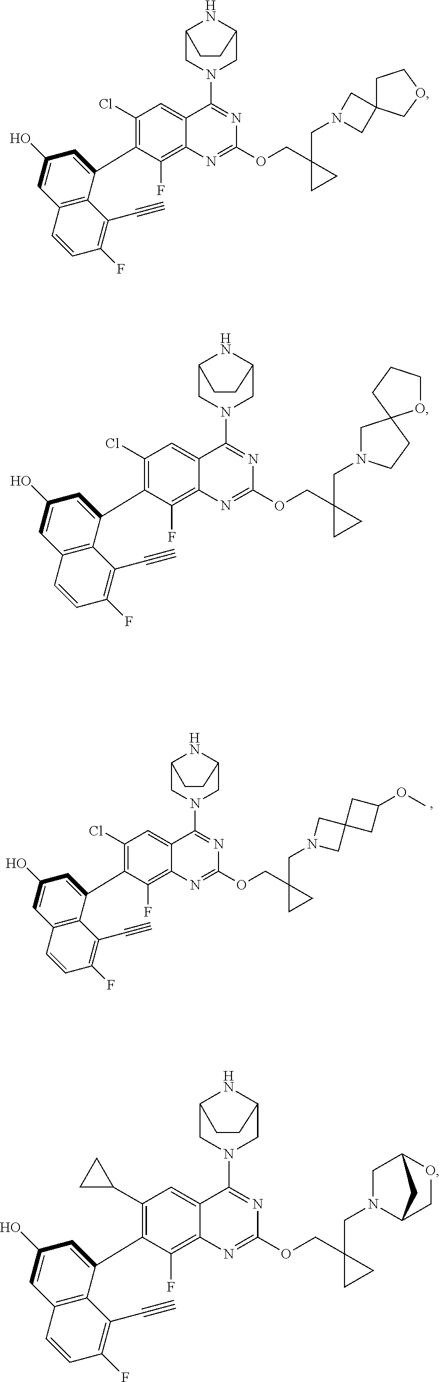

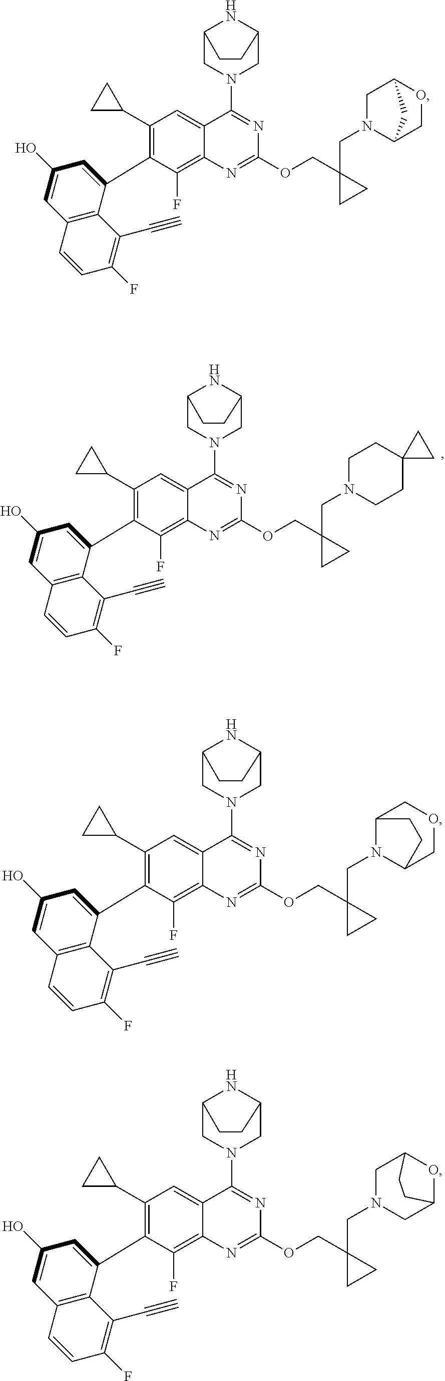

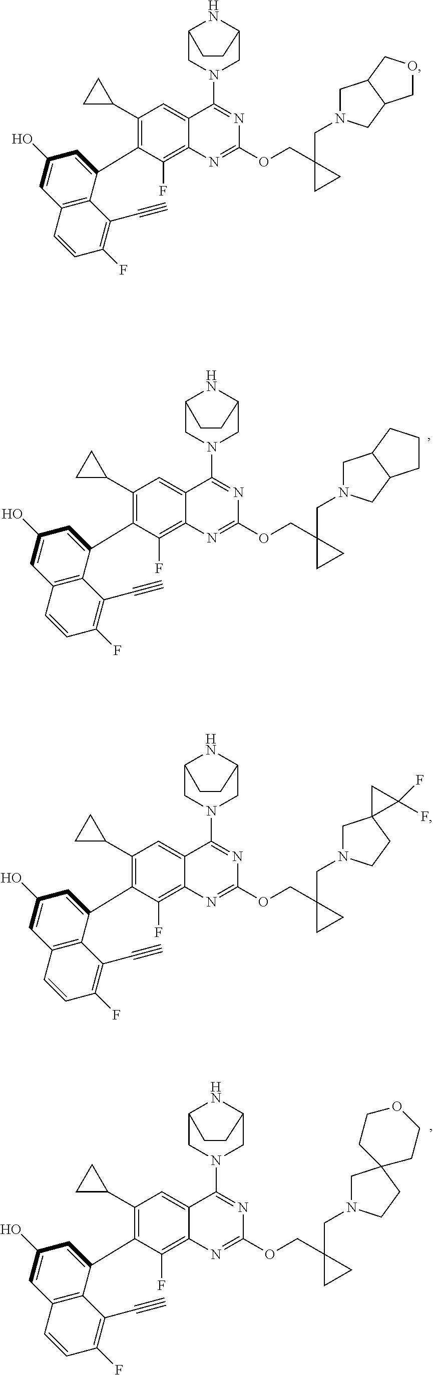

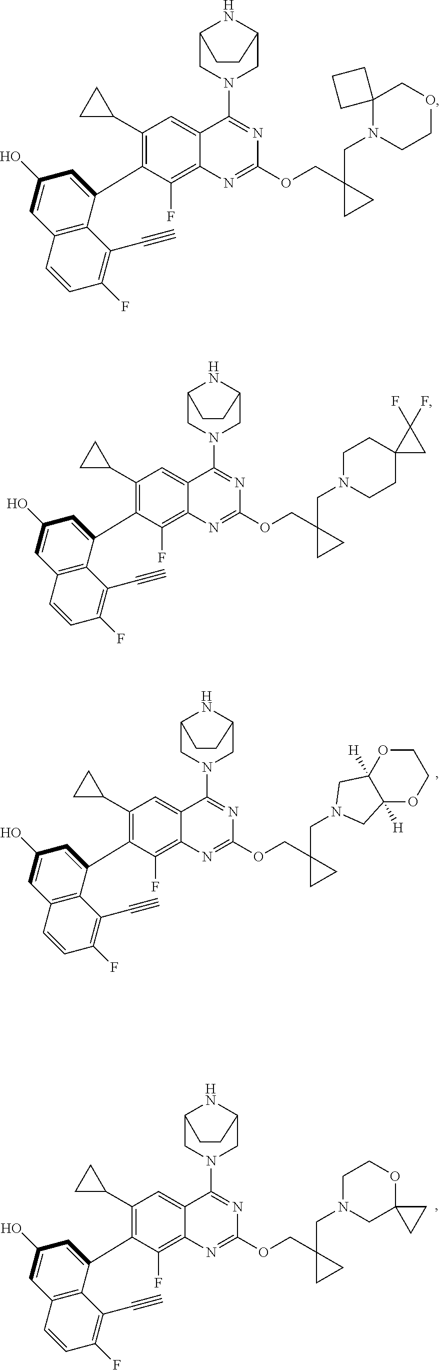

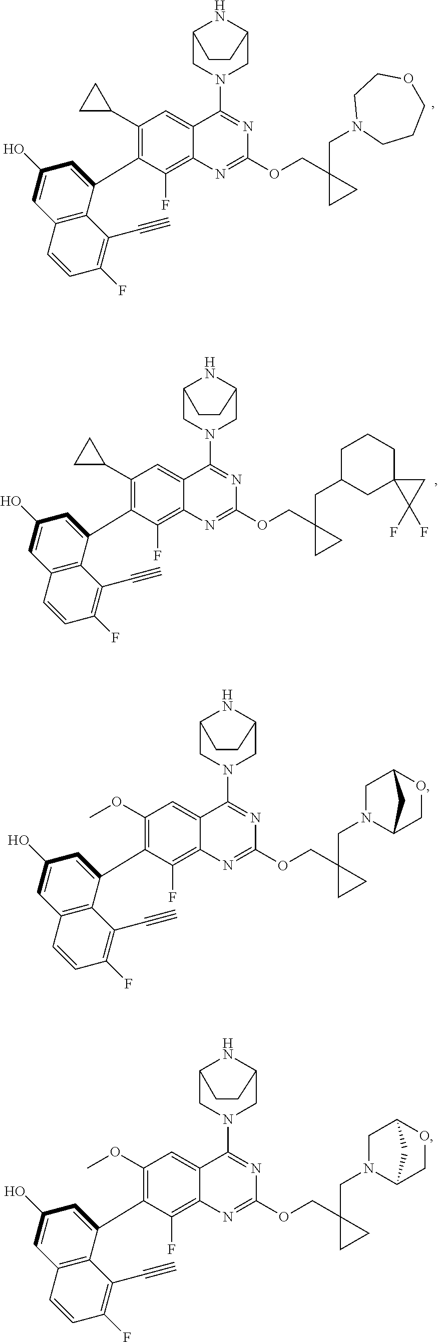

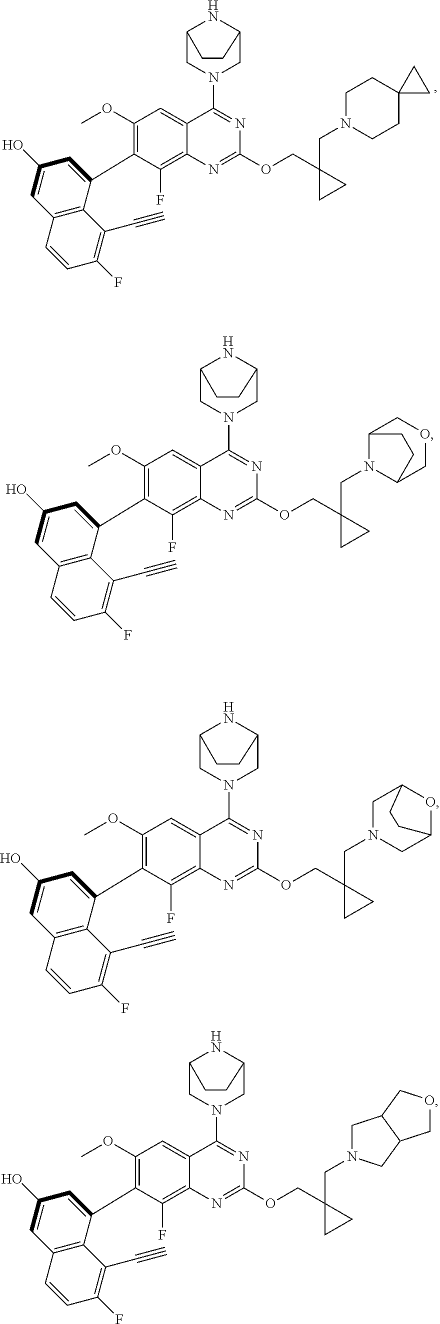

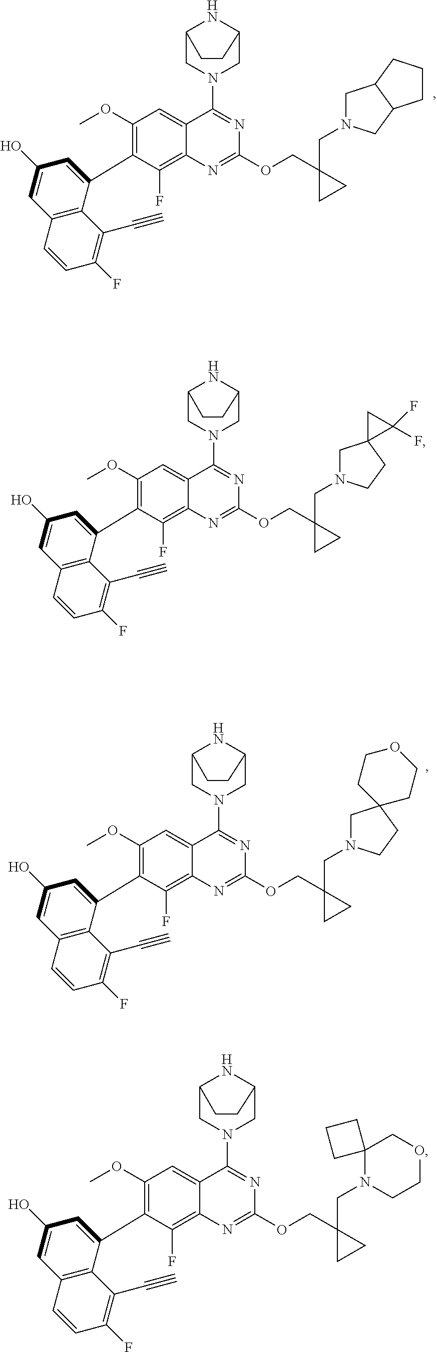

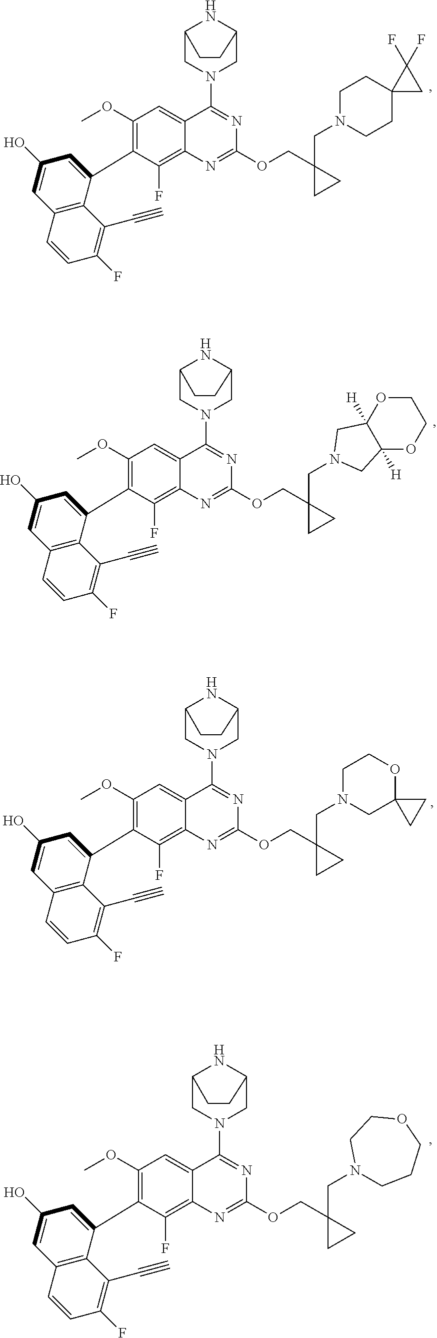

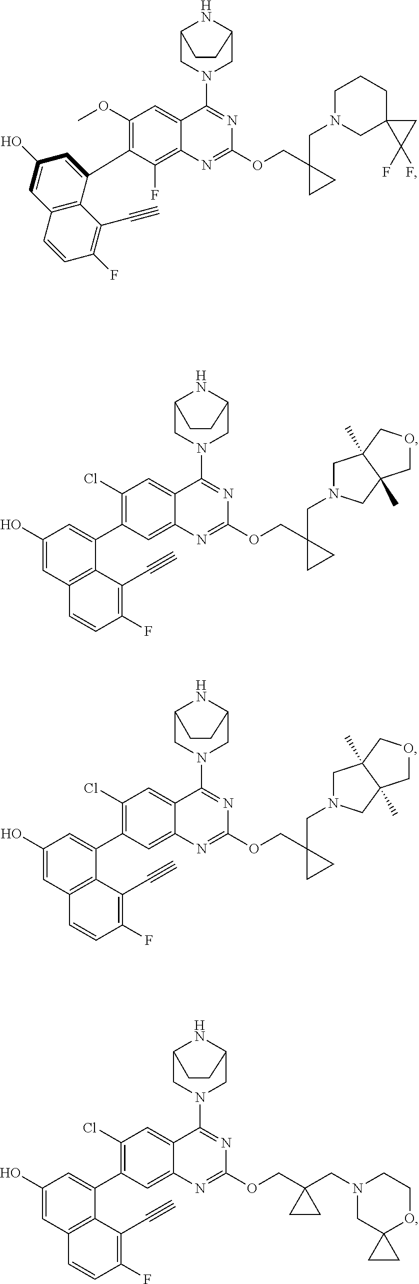

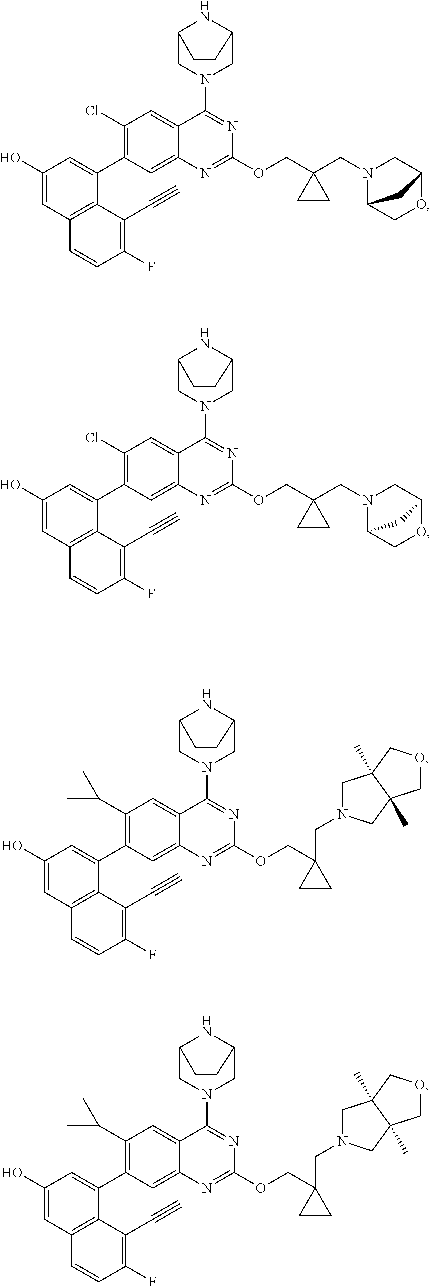 [SMILES: C#Cc1c(F)ccc2cc(O)cc(-c3cc4nc(OCC5(CN6C[C@H]7C[C@@H]6CO7)CC5)nc(N5CC6CCC(C5)N6)c4cc3Cl)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3Cl)c12 ; C#Cc1c(F)ccc2cc(O)cc(-c3cc4nc(OCC5(CN6C[C@@]7(C)COC[C@]7(C)C6)CC5)nc(N5CC6CCC(C5)N6)c4cc3C(C)C)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3C(C)C)c12]

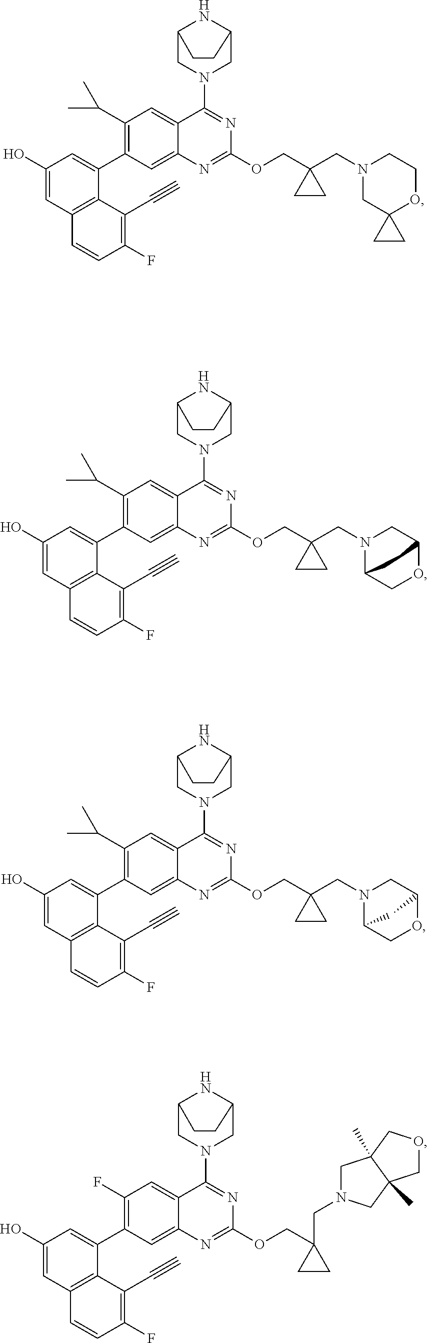

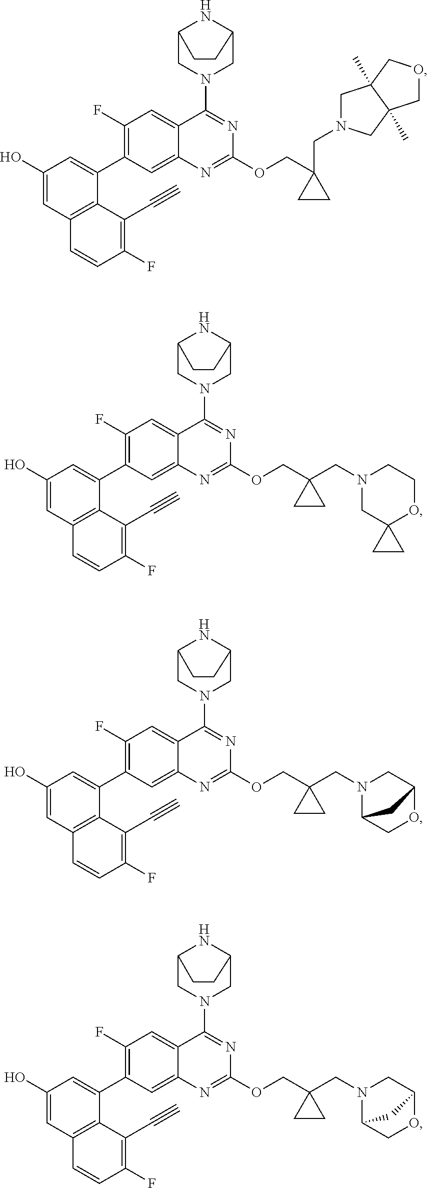 [SMILES: C#Cc1c(F)ccc2cc(O)cc(-c3cc4nc(OCC5(CN6C[C@]7(C)COC[C@]7(C)C6)CC5)nc(N5CC6CCC(C5)N6)c4cc3F)c12 ; C#Cc1c(F)ccc2cc(O)cc(-c3cc4nc(OCC5(CN6CCOC7(CC7)C6)CC5)nc(N5CC6CCC(C5)N6)c4cc3F)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3F)c12 ; C#Cc1c(F)ccc2cc(O)cc(-c3cc4nc(OCC5(CN6C[C@@H]7C[C@H]6CO7)CC5)nc(N5CC6CCC(C5)N6)c4cc3F)c12]

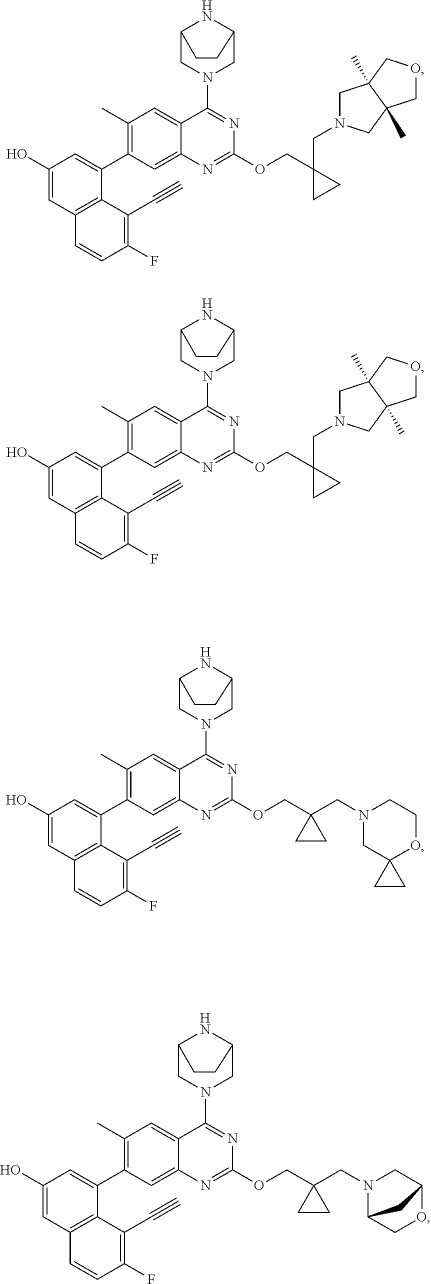 [SMILES: C#Cc1c(F)ccc2cc(O)cc(-c3cc4nc(OCC5(CN6C[C@@]7(C)COC[C@]7(C)C6)CC5)nc(N5CC6CCC(C5)N6)c4cc3C)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3C)c12 ; C#Cc1c(F)ccc2cc(O)cc(-c3cc4nc(OCC5(CN6CCOC7(CC7)C6)CC5)nc(N5CC6CCC(C5)N6)c4cc3C)c12 ; C#Cc1c(F)ccc2cc(O)cc(-c3cc4nc(OCC5(CN6C[C@H]7C[C@@H]6CO7)CC5)nc(N5CC6CCC(C5)N6)c4cc3C)c12]

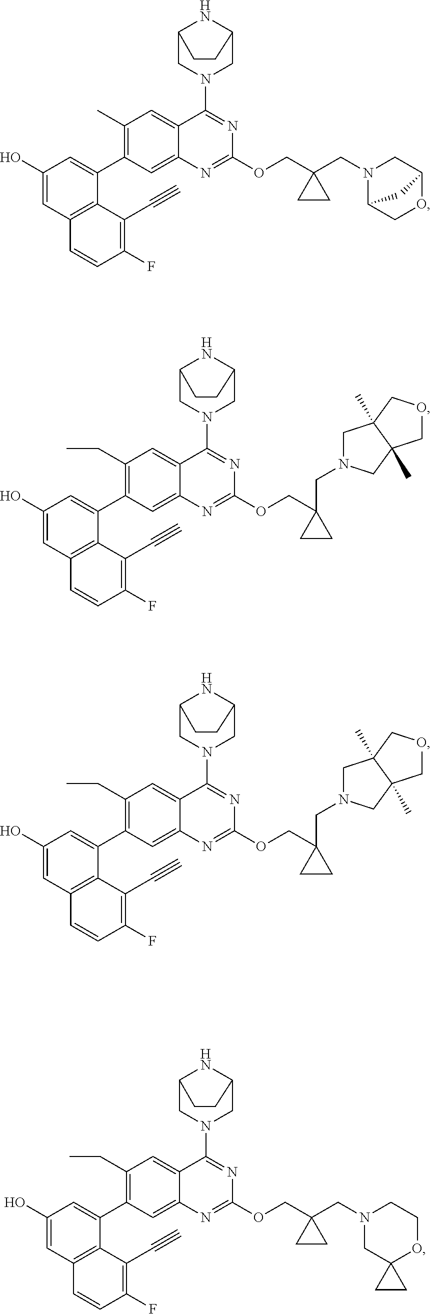 [SMILES: C#Cc1c(F)ccc2cc(O)cc(-c3cc4nc(OCC5(CN6C[C@@H]7C[C@H]6CO7)CC5)nc(N5CC6CCC(C5)N6)c4cc3C)c12 ; C#Cc1c(F)ccc2cc(O)cc(-c3cc4nc(OCC5(CN6C[C@@]7(C)COC[C@]7(C)C6)CC5)nc(N5CC6CCC(C5)N6)c4cc3CC)c12 ; C#Cc1c(F)ccc2cc(O)cc(-c3cc4nc(OCC5(CN6C[C@]7(C)COC[C@]7(C)C6)CC5)nc(N5CC6CCC(C5)N6)c4cc3CC)c12 ; C#Cc1c(F)ccc2cc(O)cc(-c3cc4nc(OCC5(CN6CCOC7(CC7)C6)CC5)nc(N5CC6CCC(C5)N6)c4cc3CC)c12]

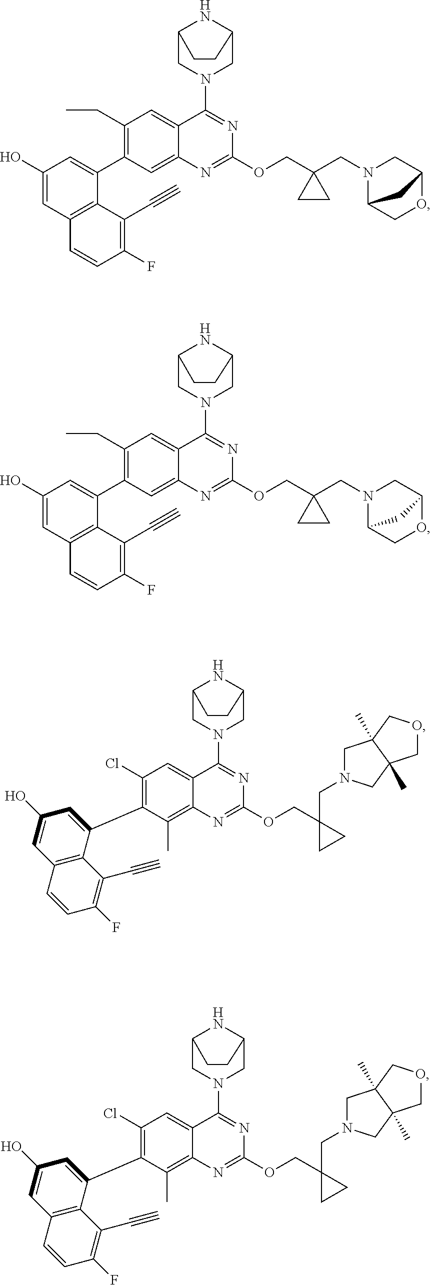

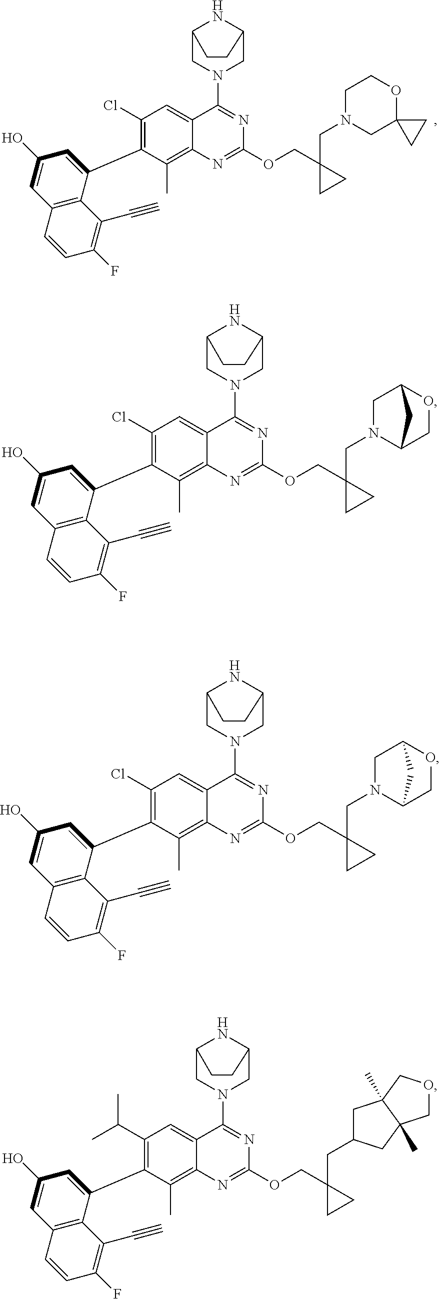

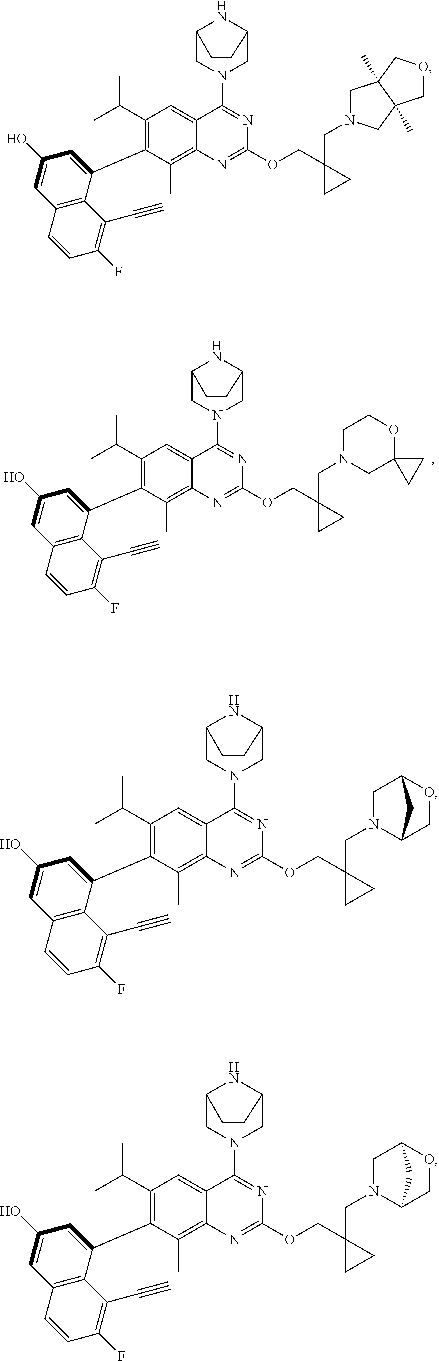

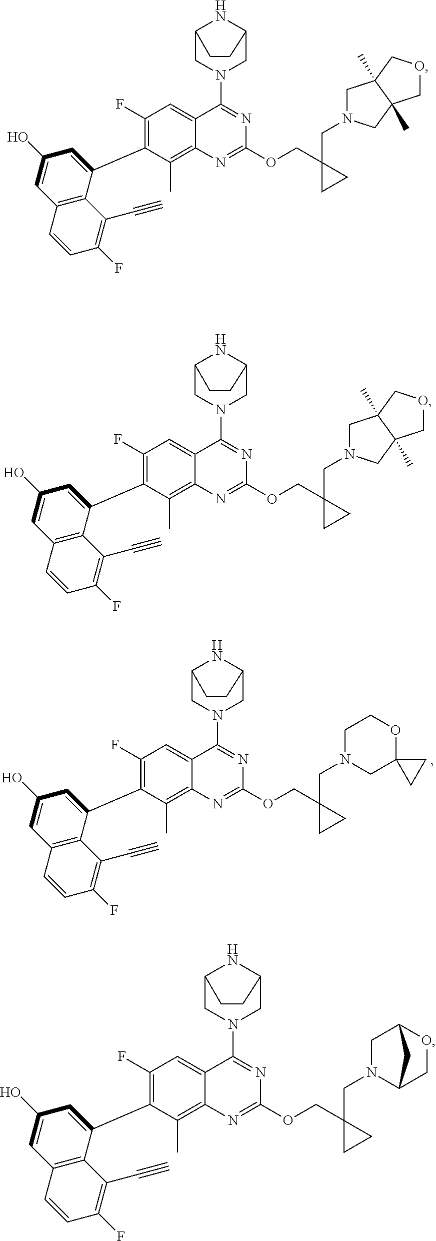

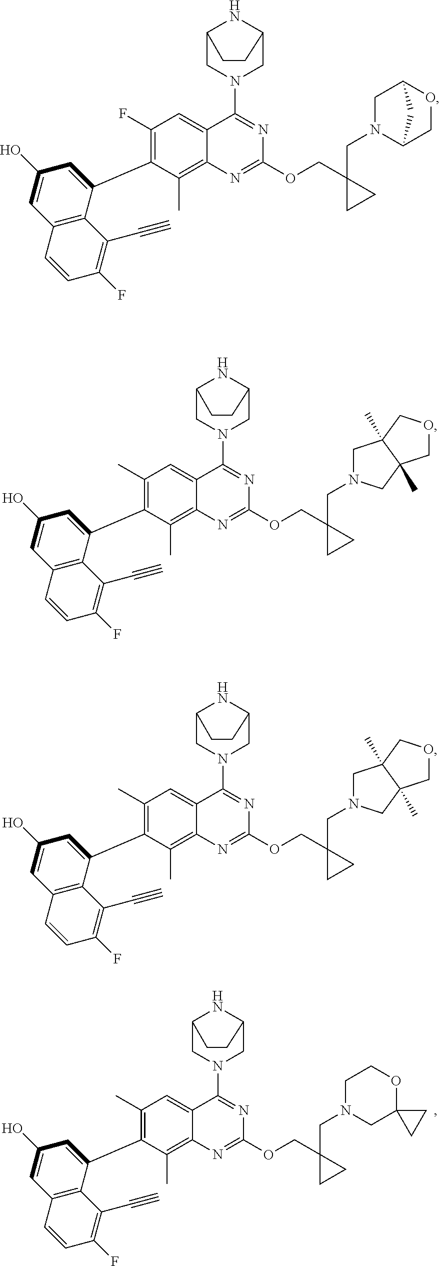

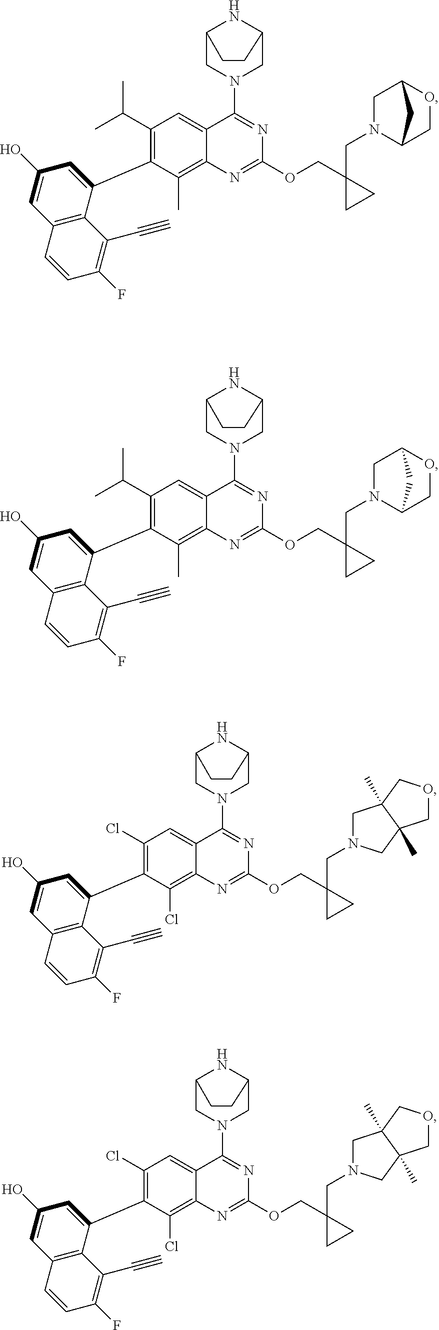

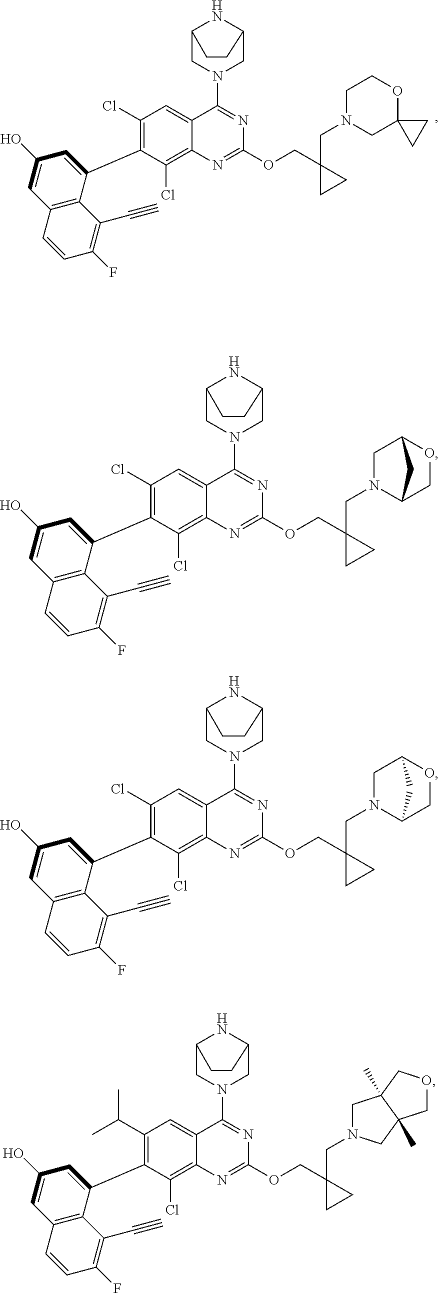

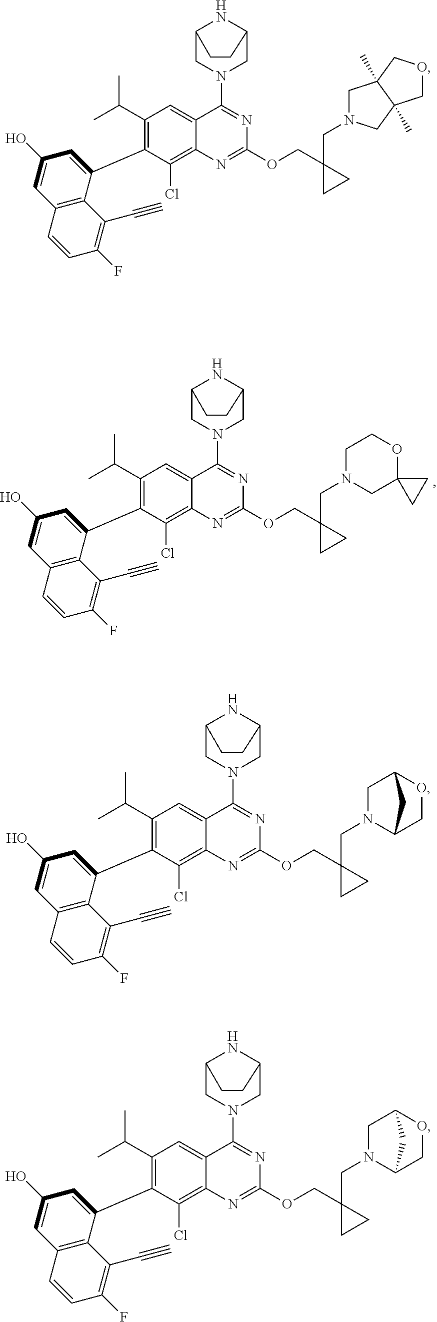

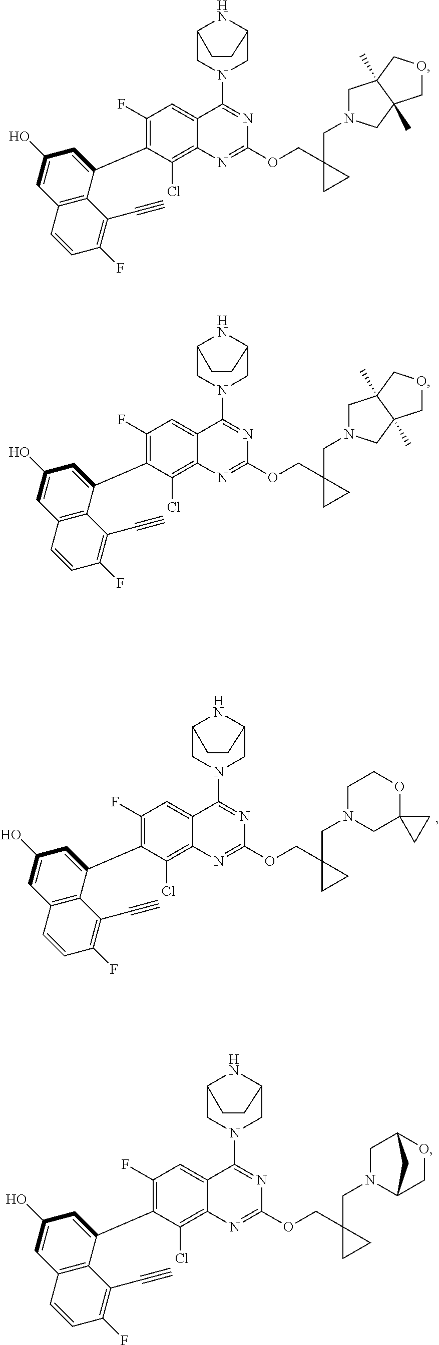

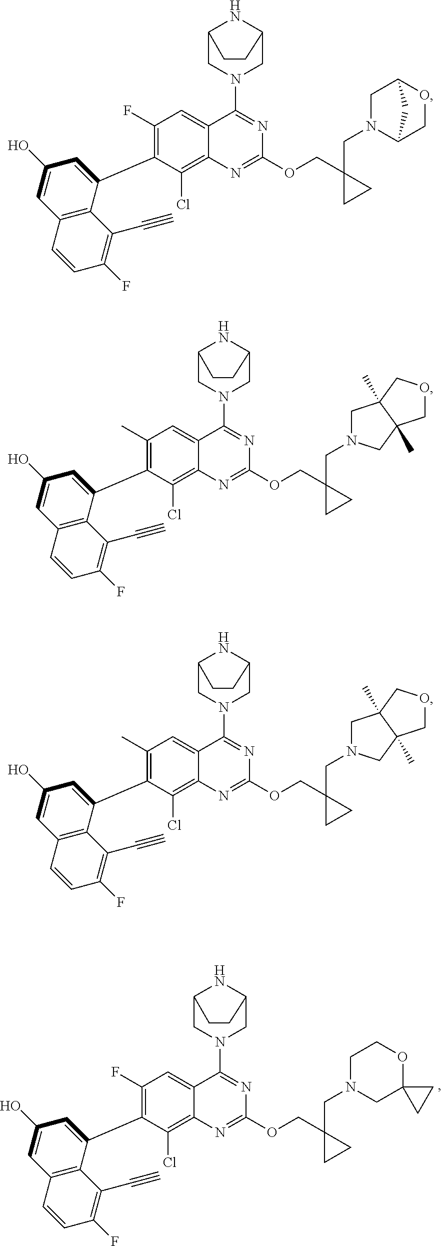

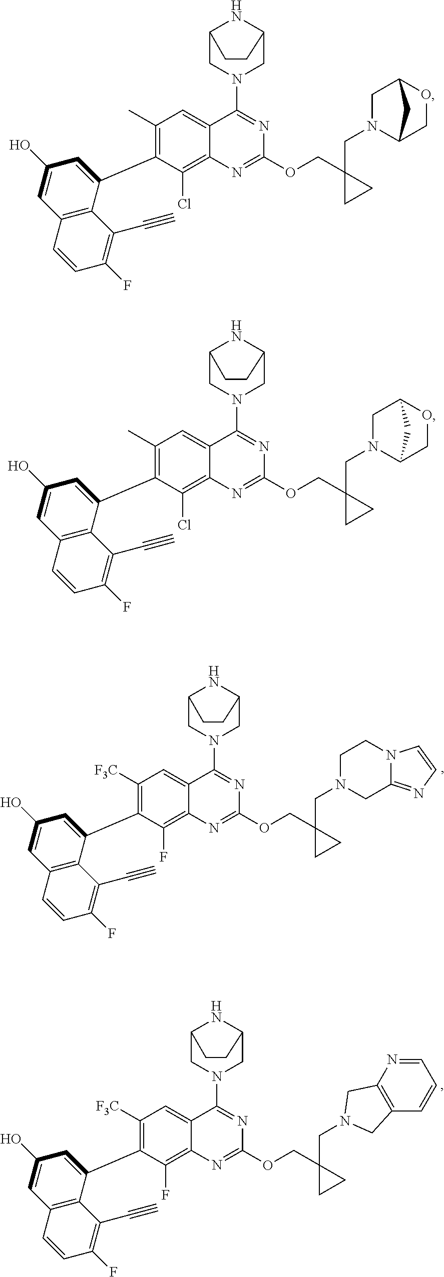

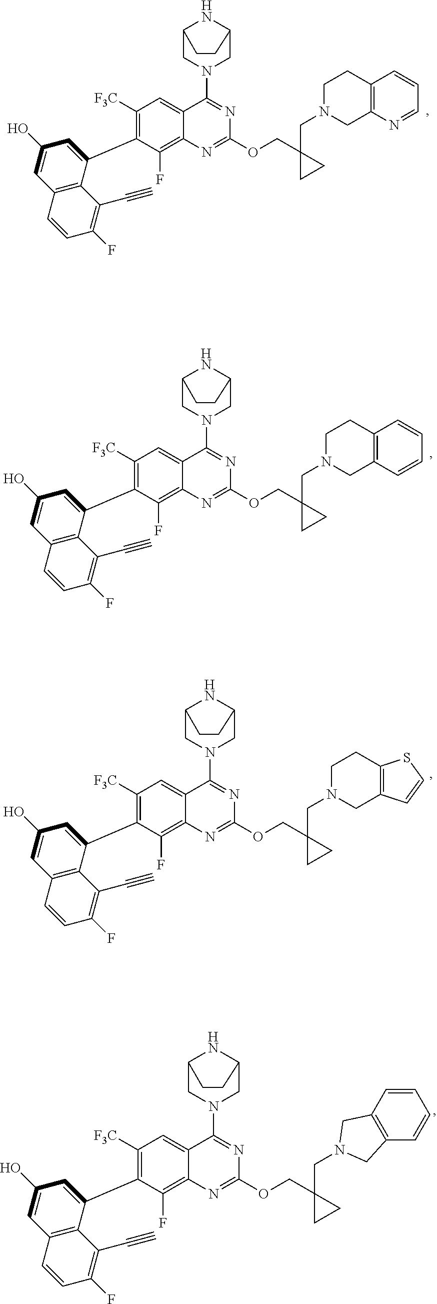

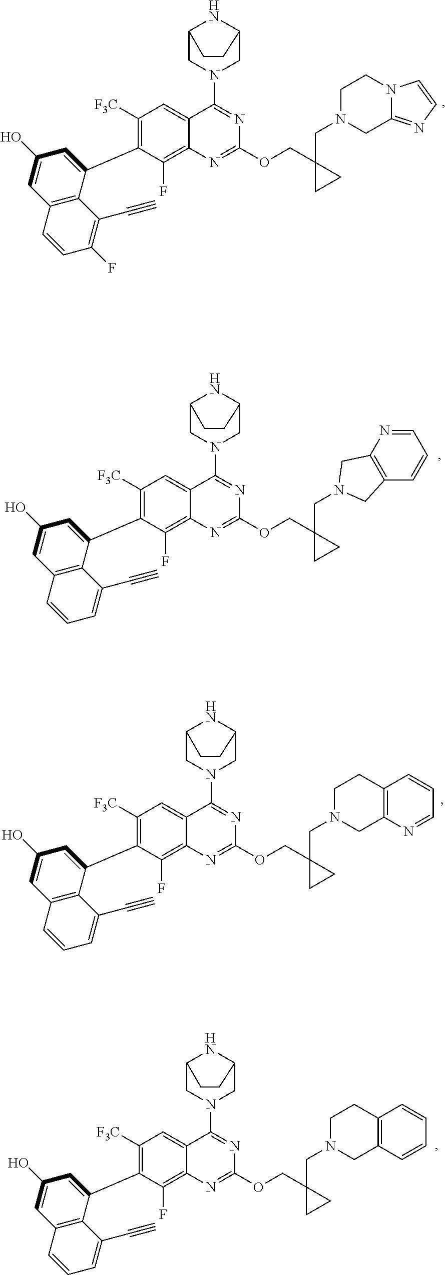

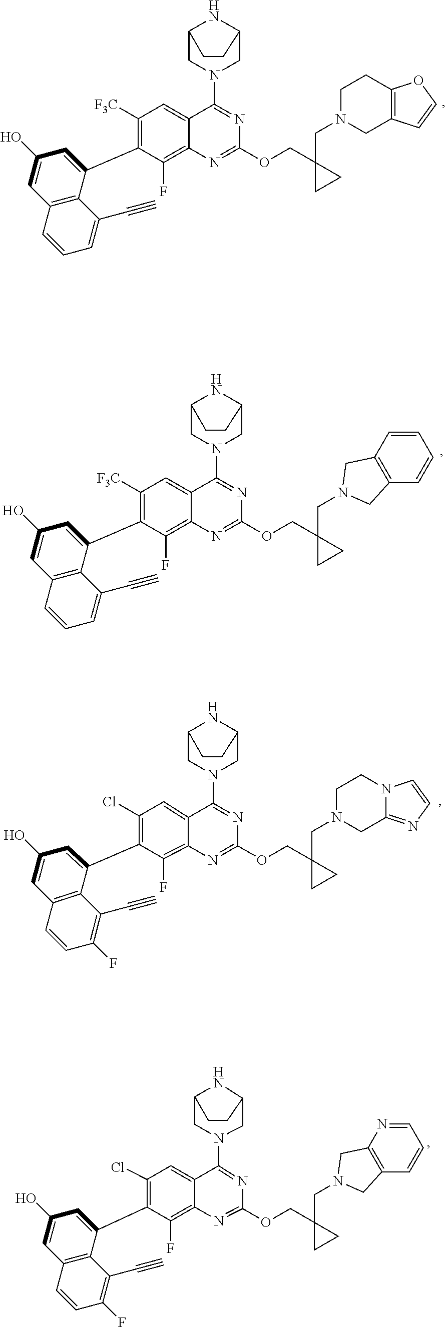

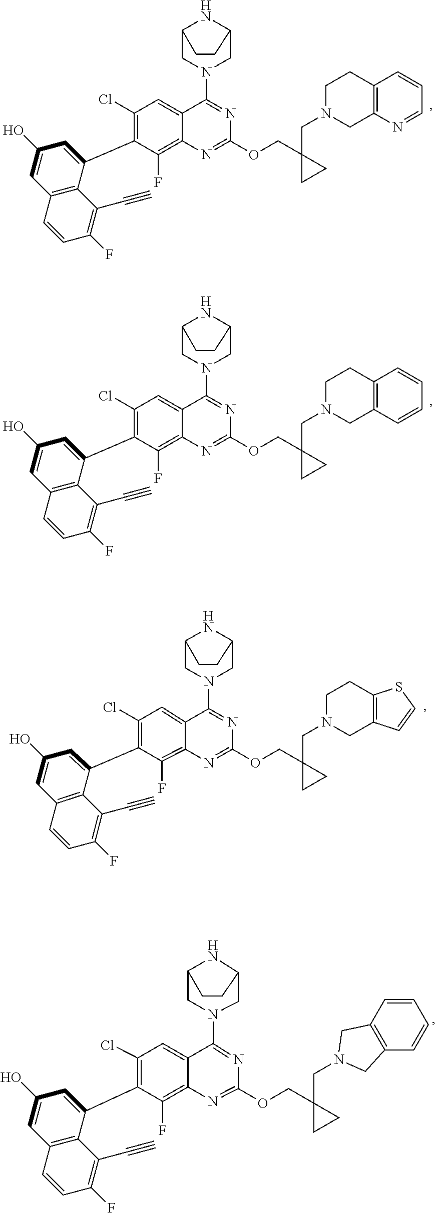

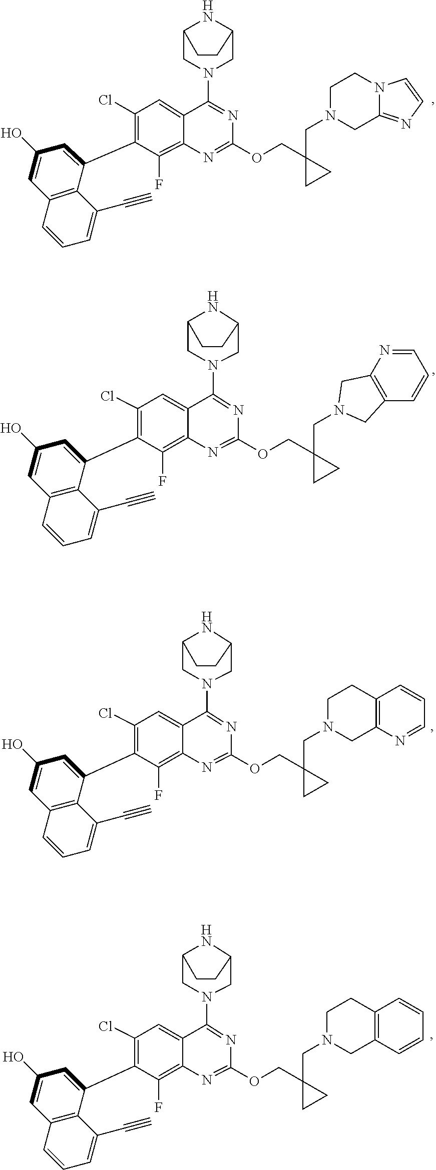

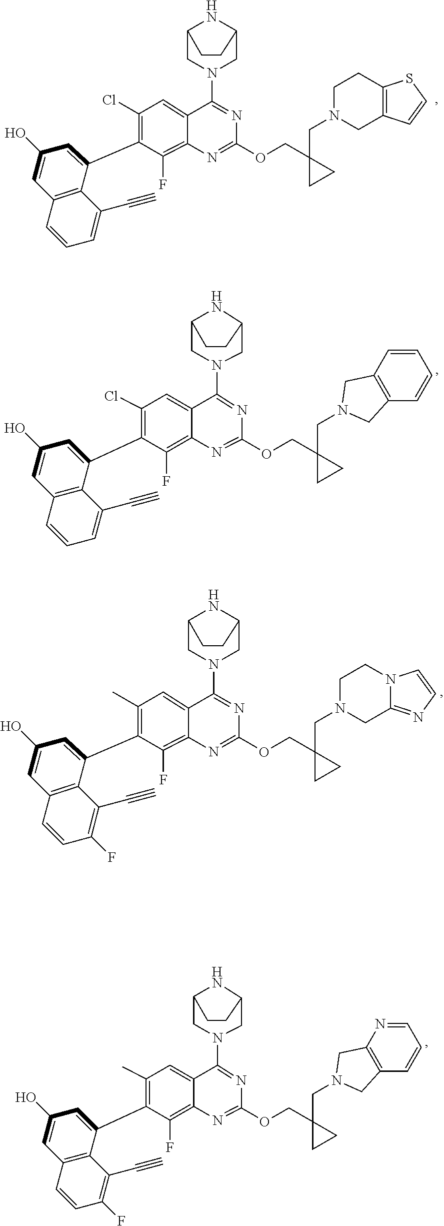

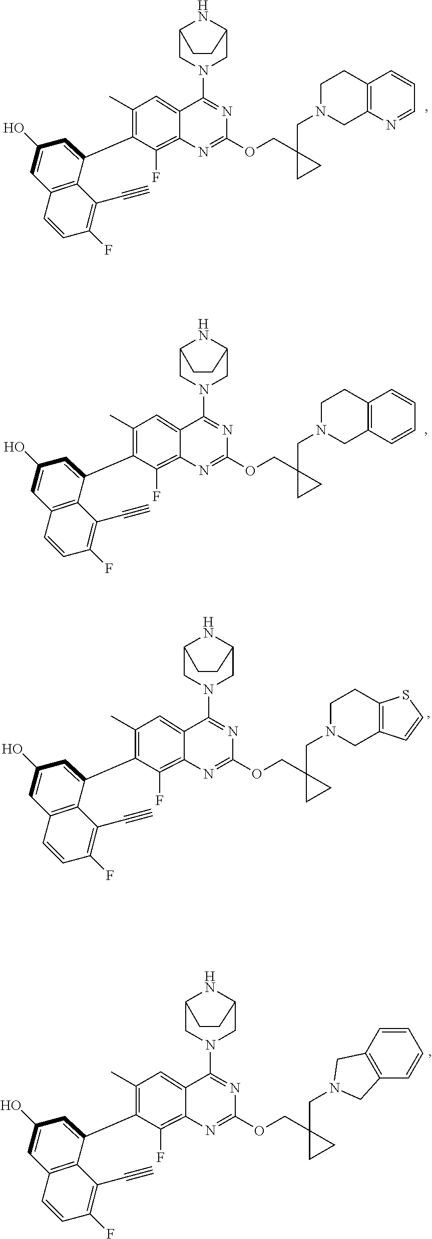

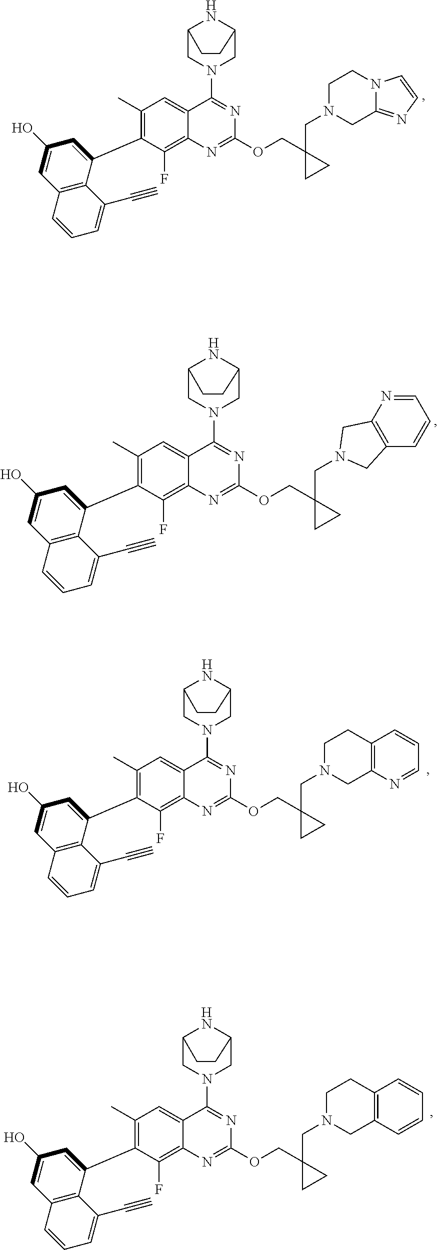

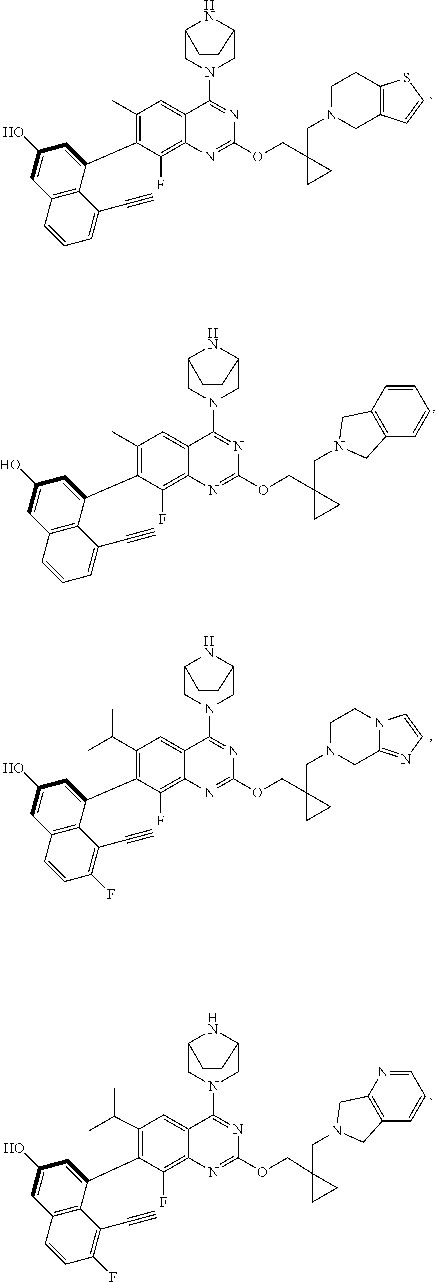

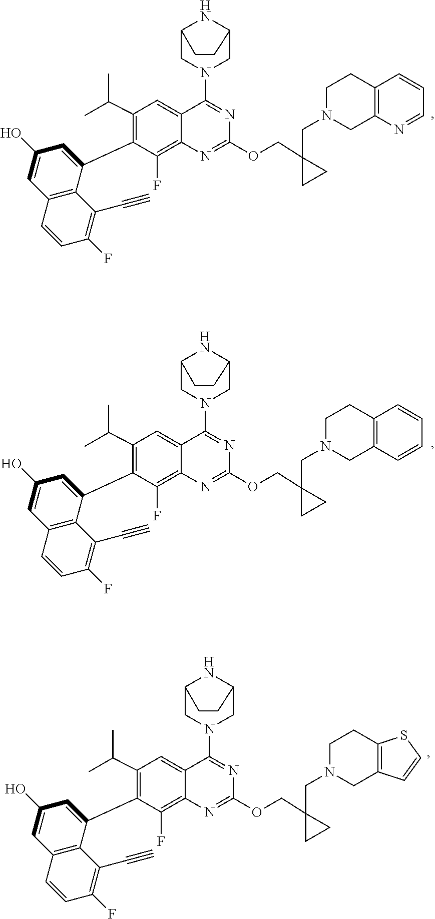

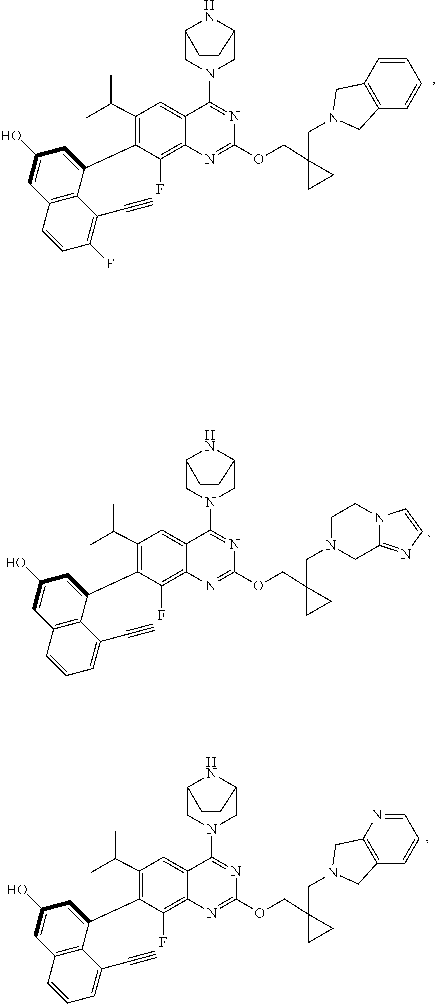

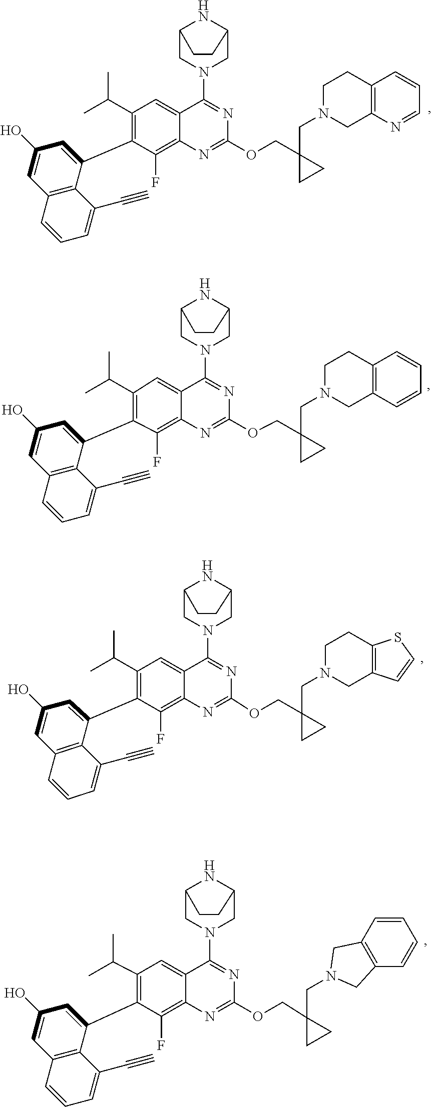

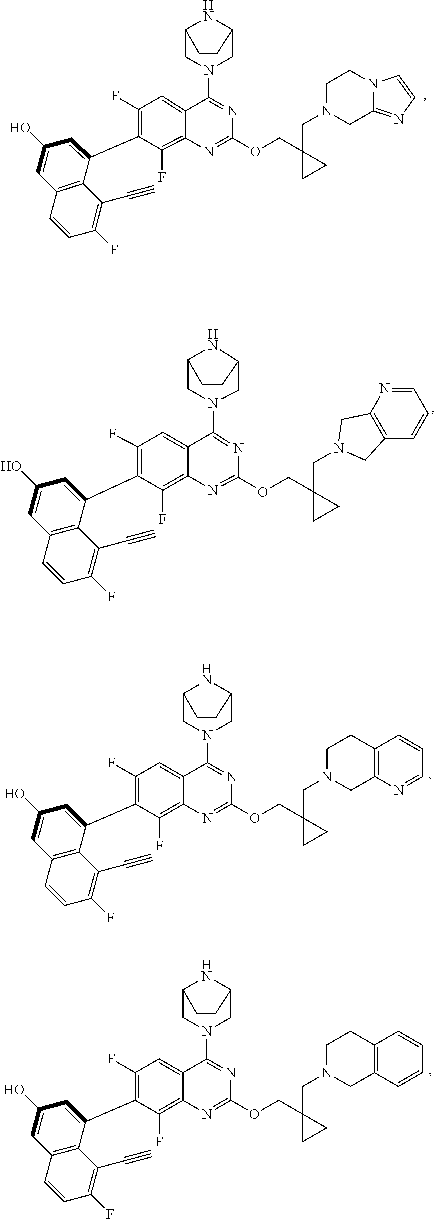

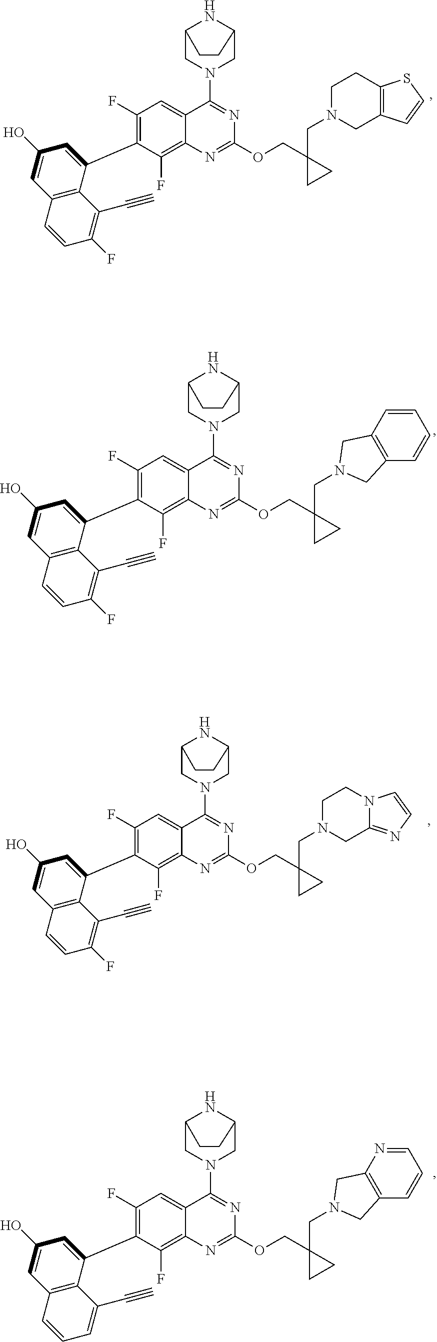

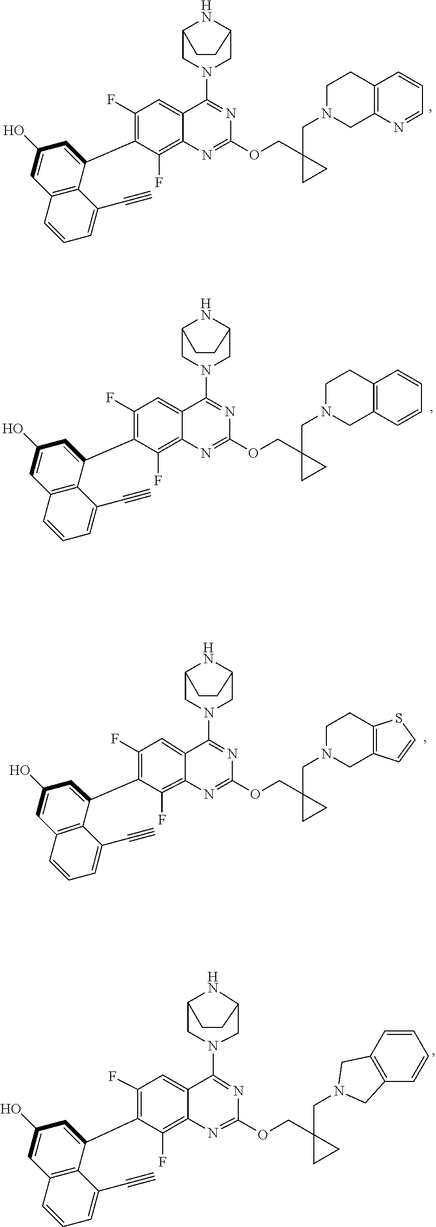

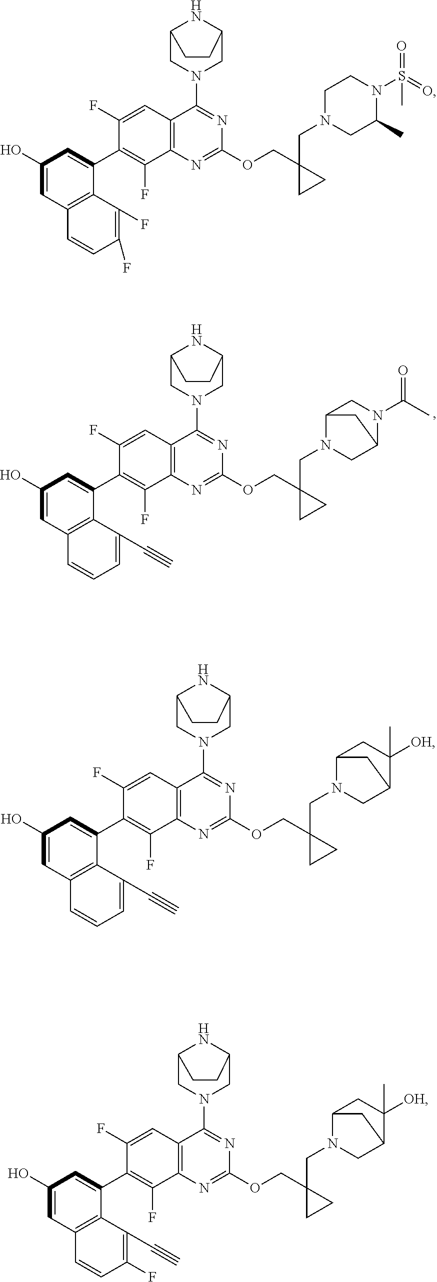

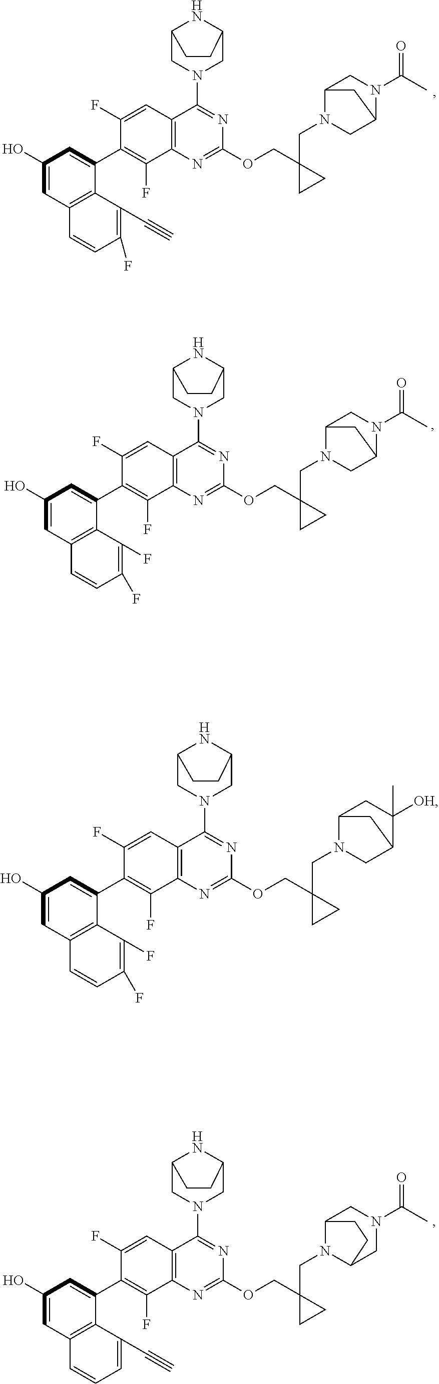

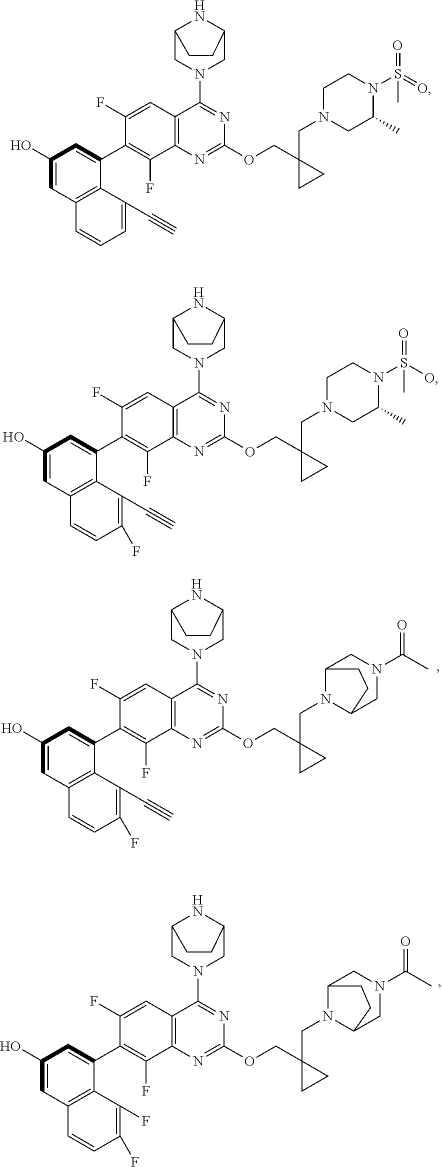

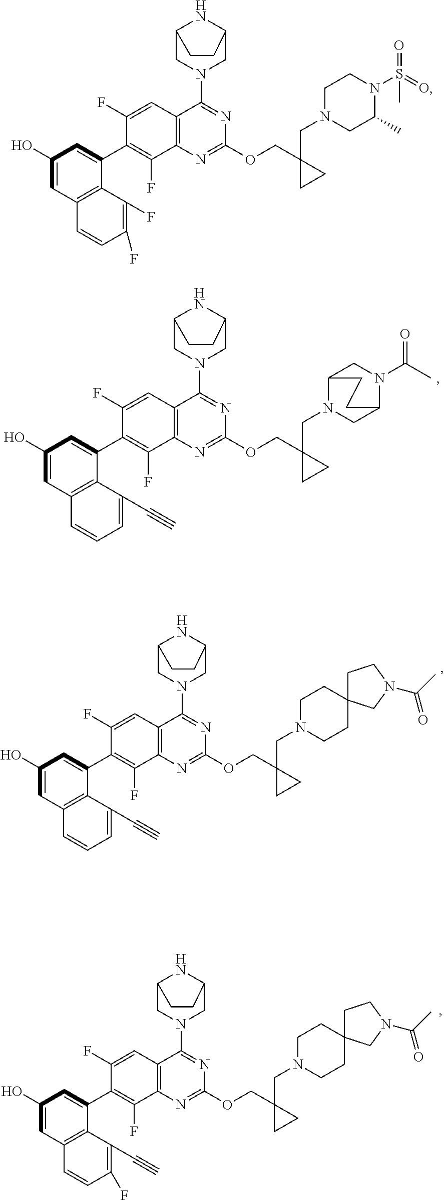

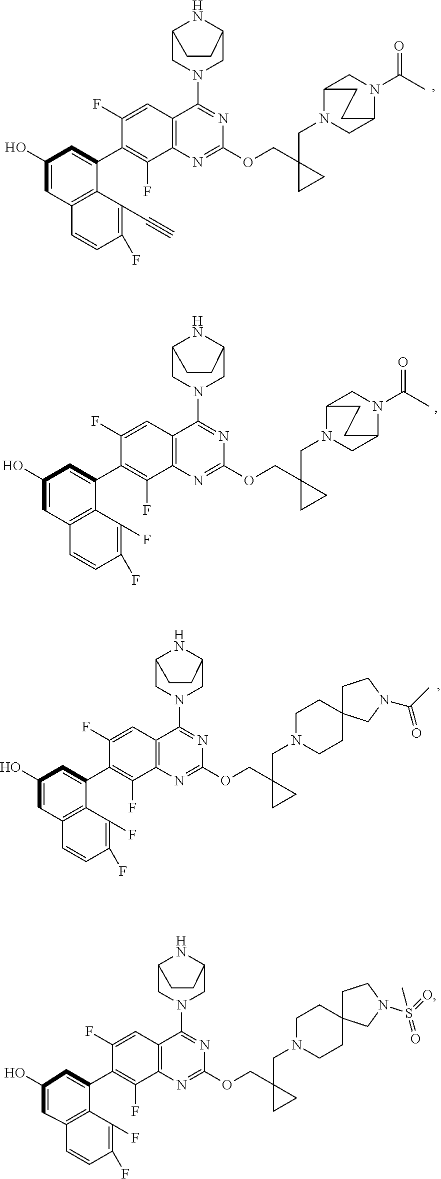

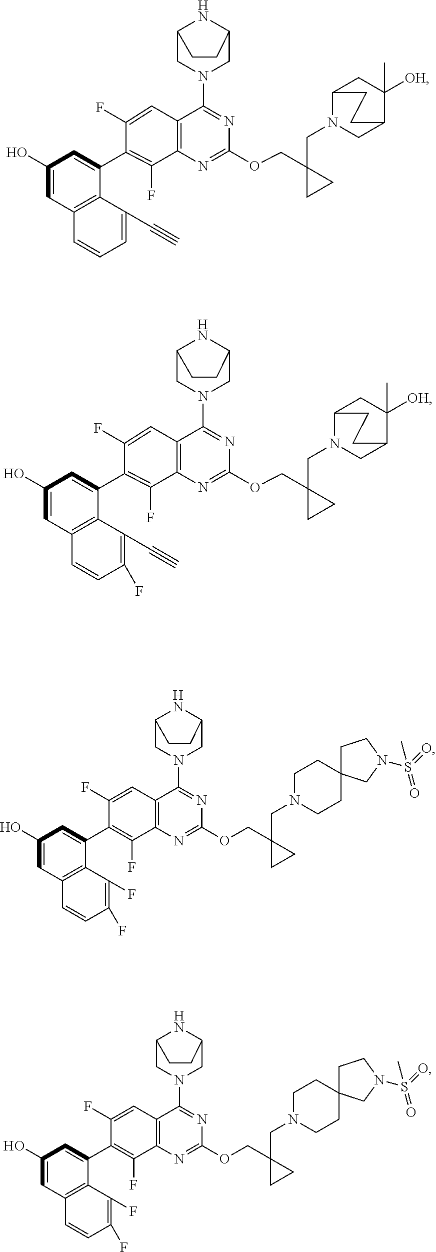

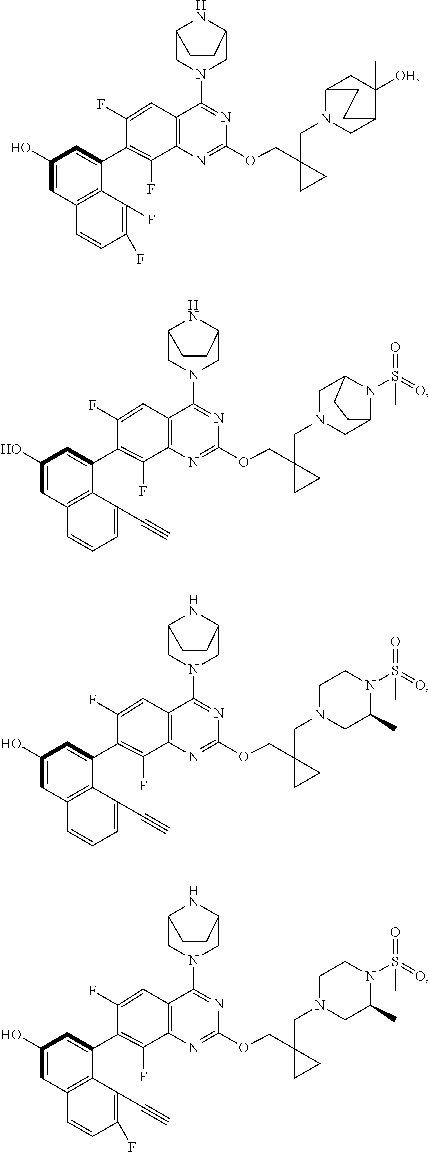

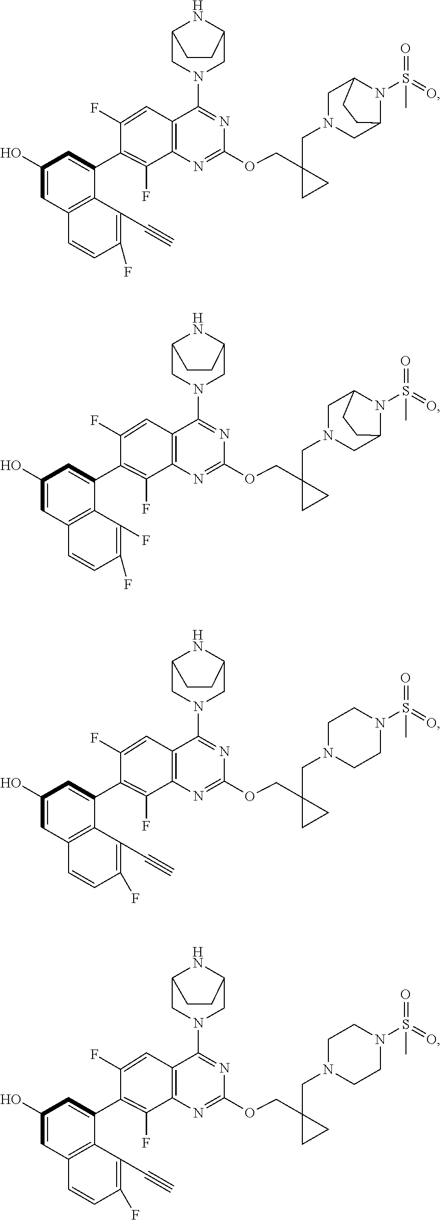

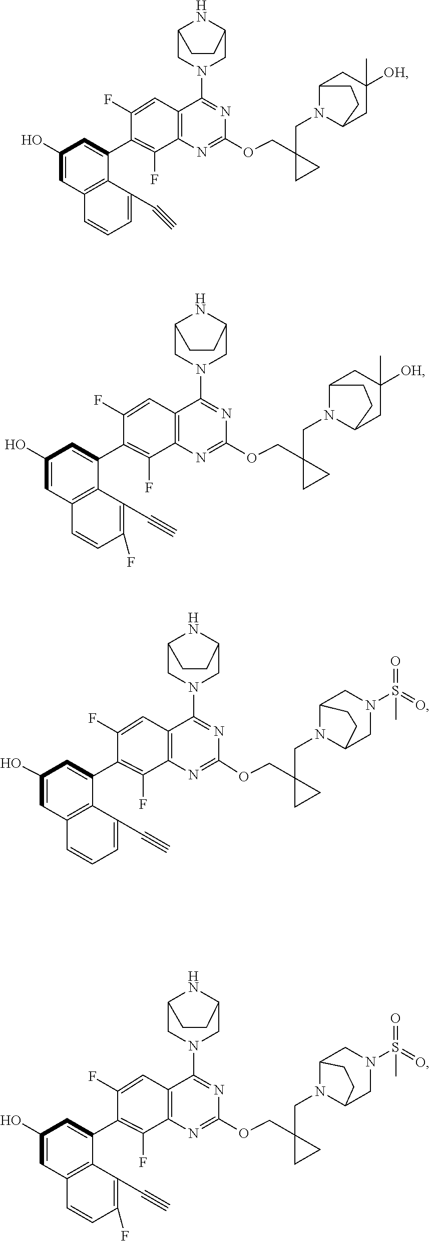

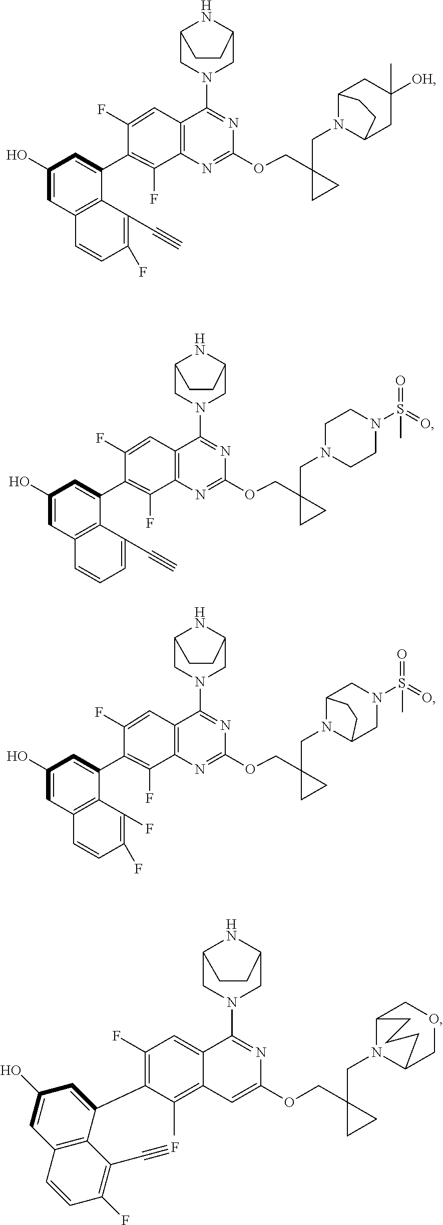

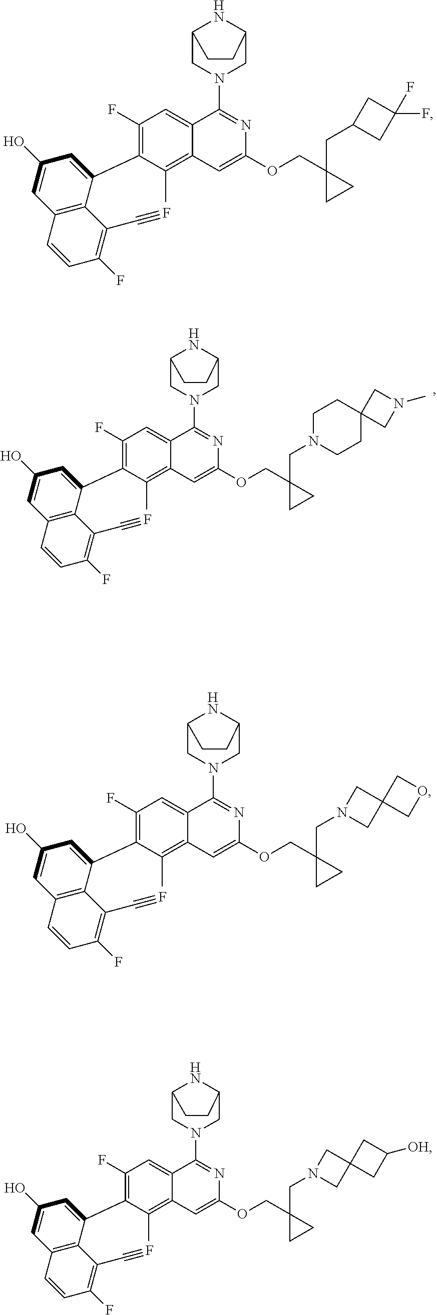

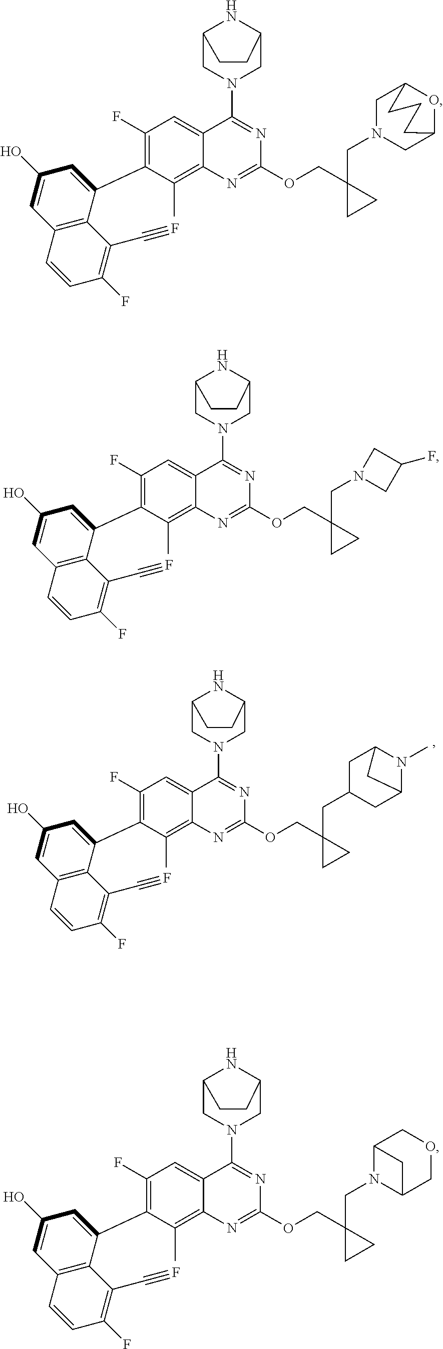

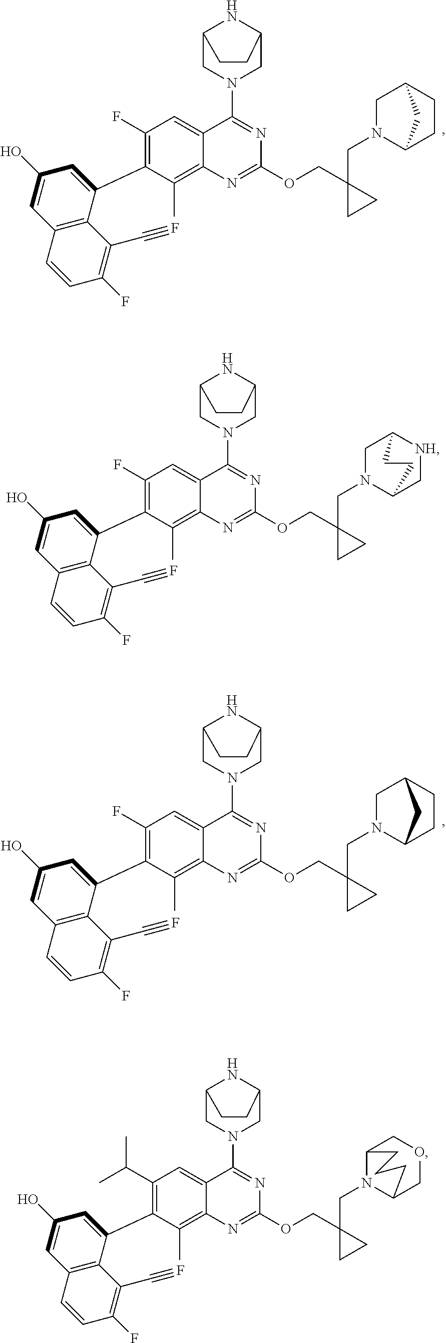

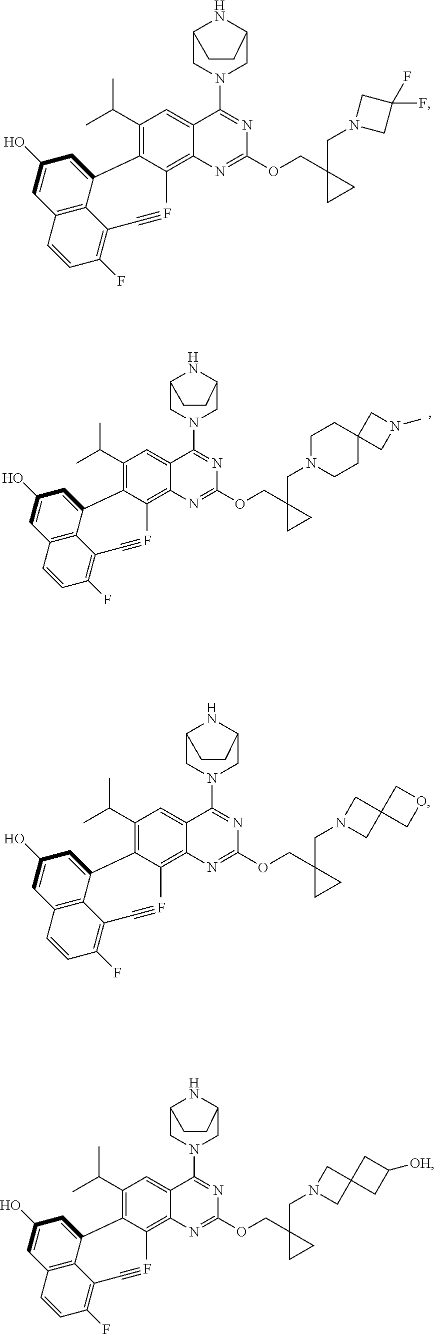

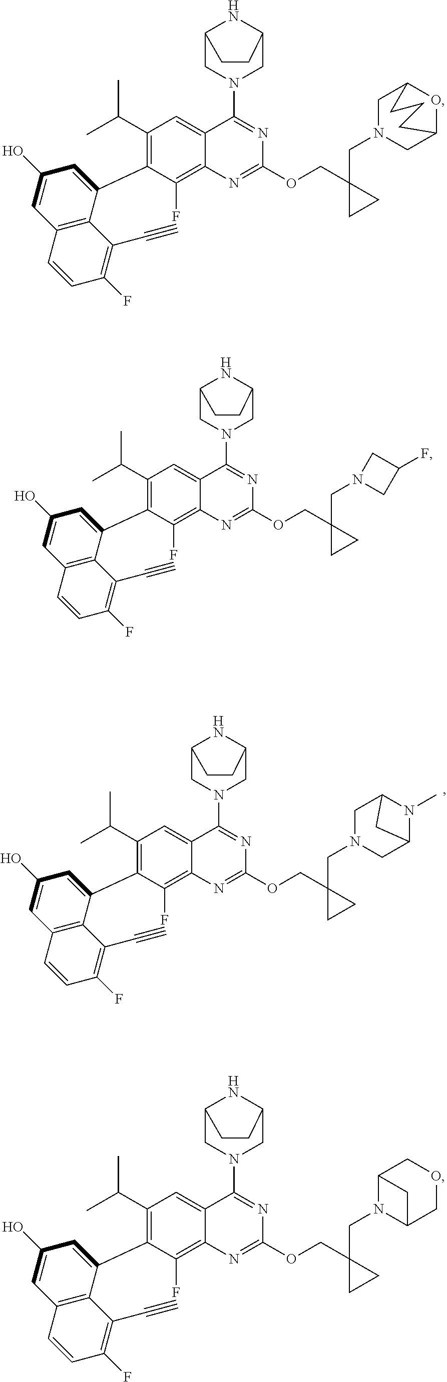

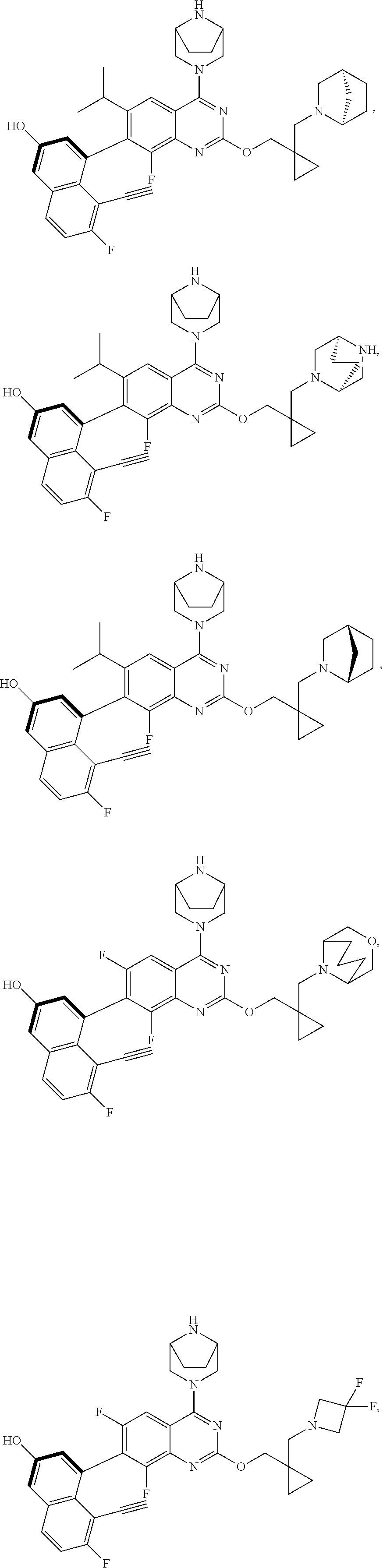

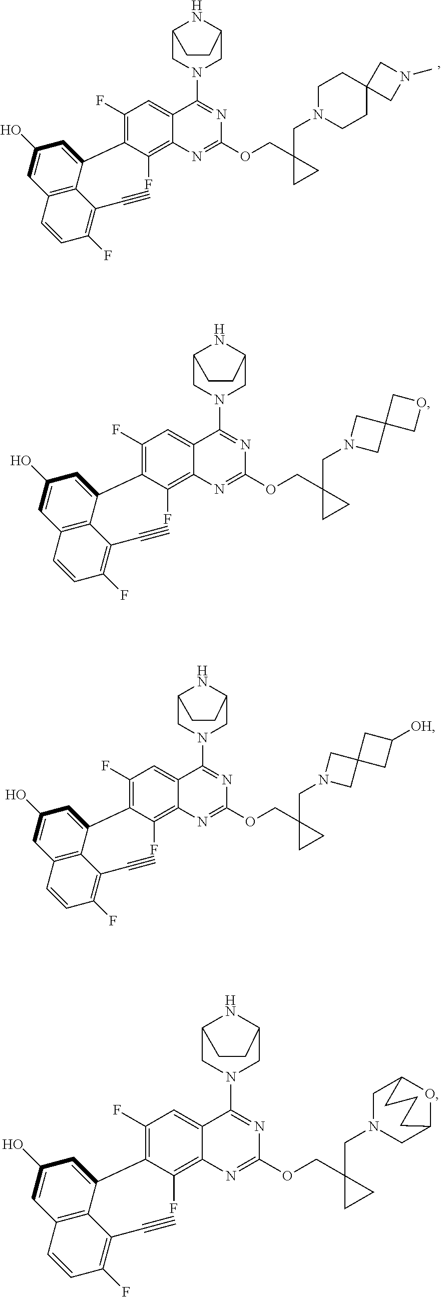

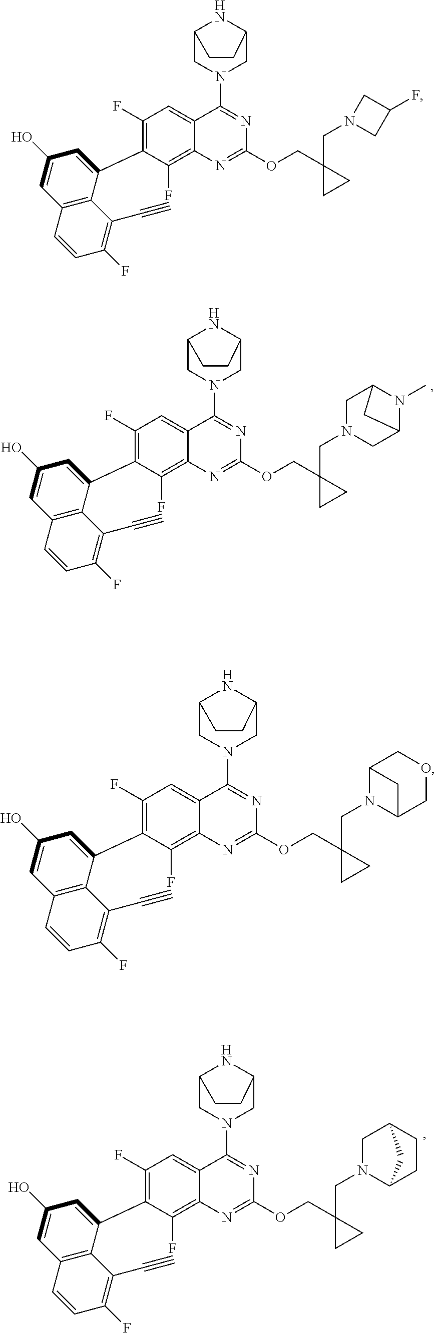

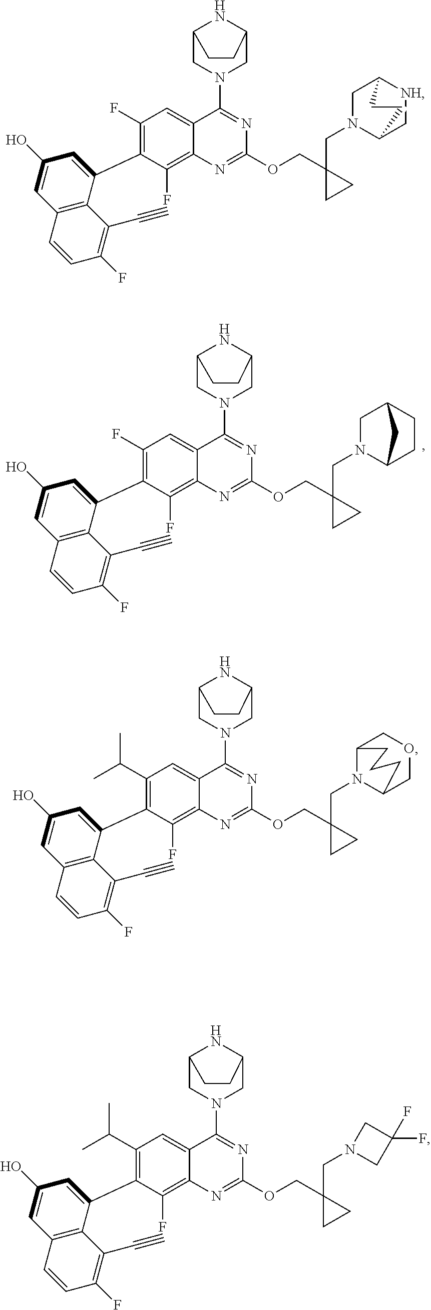

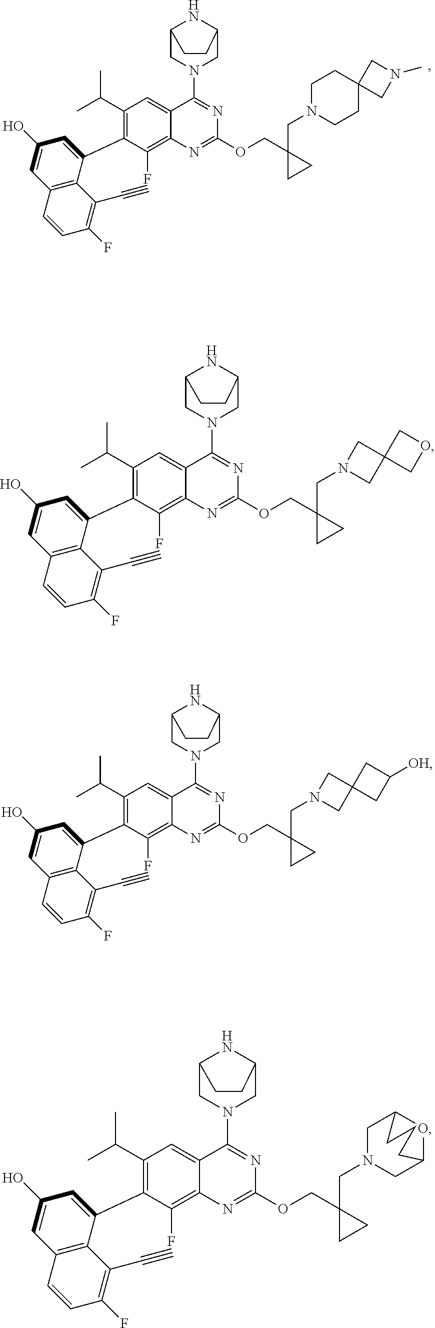

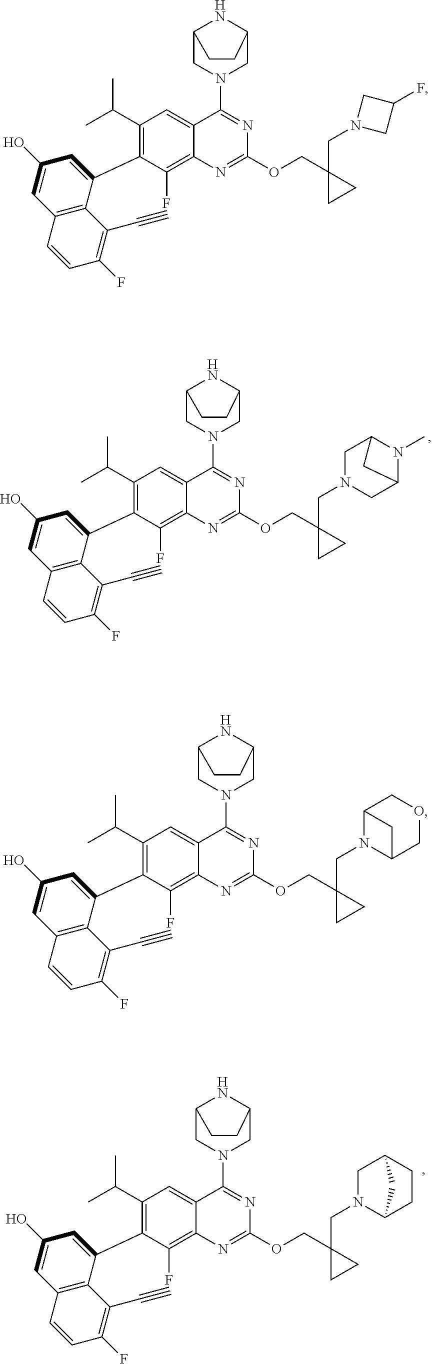

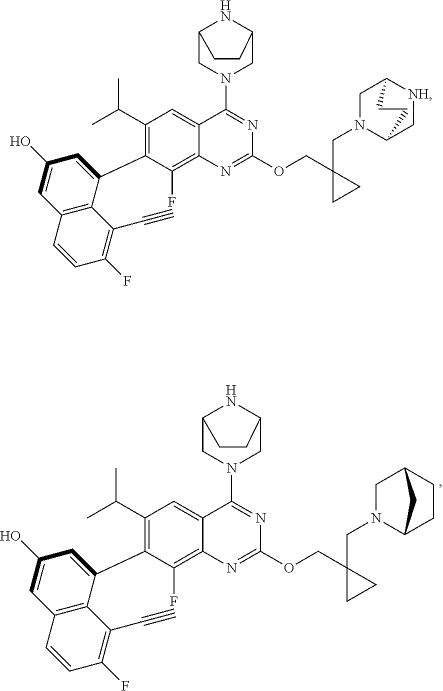

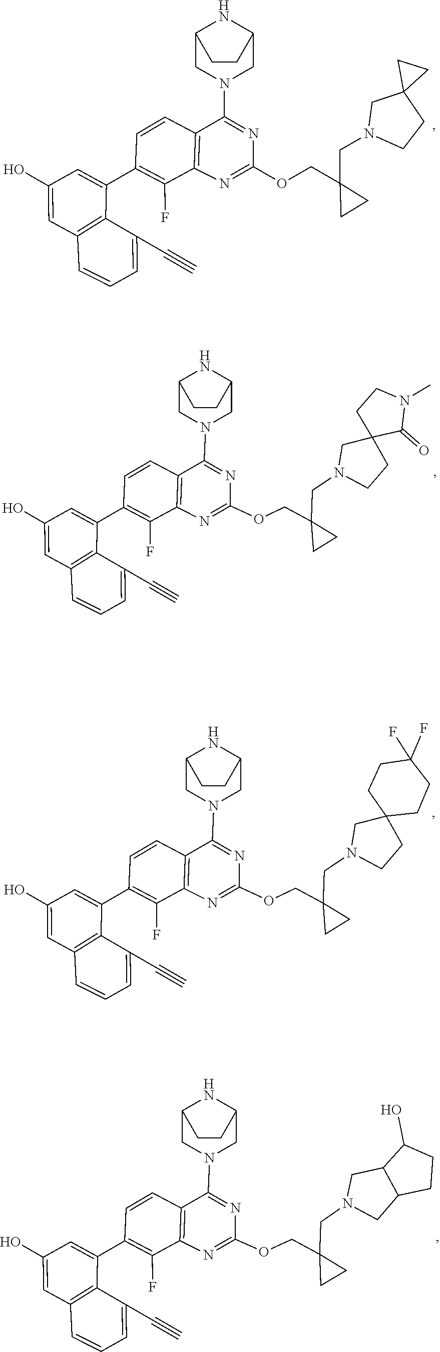 [SMILES: C#Cc1cccc2cc(O)cc(-c3ccc4c(N5CC6CCC(C5)N6)nc(OCC5(CN6CCC7(CC7)C6)CC5)nc4c3F)c12 ; C#Cc1cccc2cc(O)cc(-c3ccc4c(N5CC6CCC(C5)N6)nc(OCC5(CN6CCC7(CCN(C)C7=O)C6)CC5)nc4c3F)c12 ; C#Cc1cccc2cc(O)cc(-c3ccc4c(N5CC6CCC(C5)N6)nc(OCC5(CN6CCC7(CCC(F)(F)CC7)C6)CC5)nc4c3F)c12 ; C#Cc1cccc2cc(O)cc(-c3ccc4c(N5CC6CCC(C5)N6)nc(OCC5(CN6CC7CCC(O)C7C6)CC5)nc4c3F)c12]

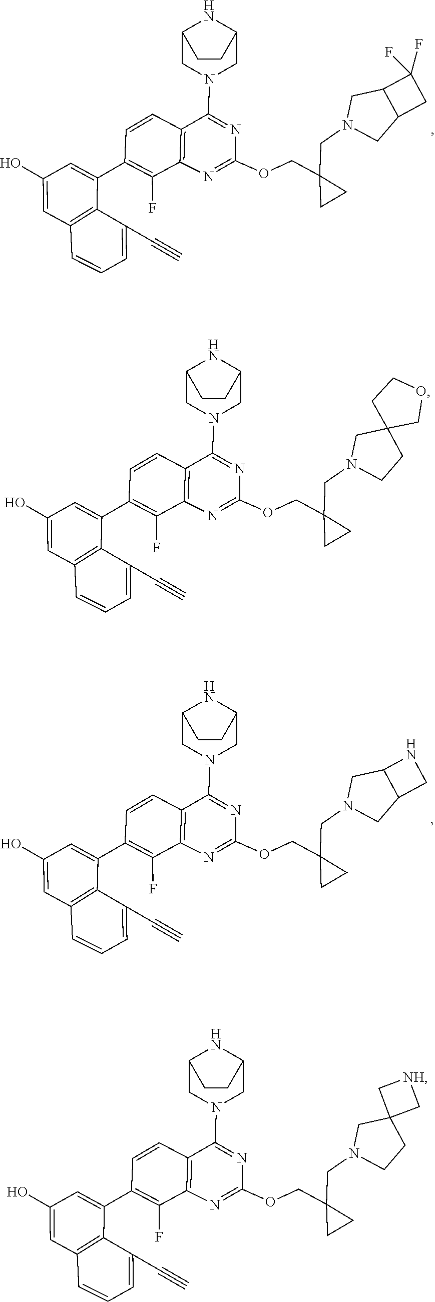 [SMILES: C#Cc1cccc2cc(O)cc(-c3ccc4c(N5CC6CCC(C5)N6)nc(OCC5(CN6CC7CC(F)(F)C7C6)CC5)nc4c3F)c12 ; C#Cc1cccc2cc(O)cc(-c3ccc4c(N5CC6CCC(C5)N6)nc(OCC5(CN6CCC7(CCOC7)C6)CC5)nc4c3F)c12 ; C#Cc1cccc2cc(O)cc(-c3ccc4c(N5CC6CCC(C5)N6)nc(OCC5(CN6CC7CNC7C6)CC5)nc4c3F)c12 ; C#Cc1cccc2cc(O)cc(-c3ccc4c(N5CC6CCC(C5)N6)nc(OCC5(CN6CCC7(CNC7)C6)CC5)nc4c3F)c12]

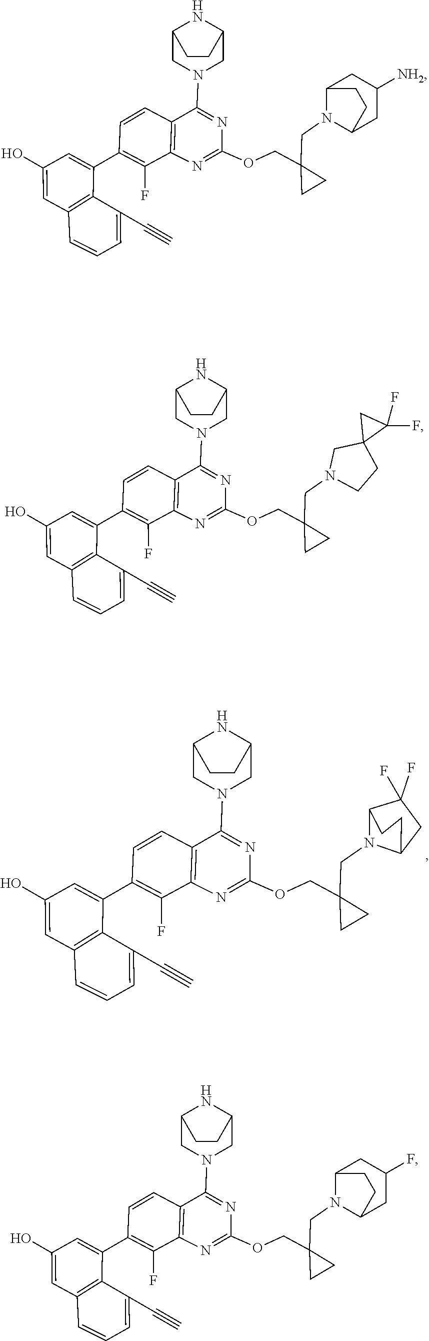 [SMILES: C#Cc1cccc2cc(O)cc(-c3ccc4c(N5CC6CCC(C5)N6)nc(OCC5(CN6C7CCC6CC(N)C7)CC5)nc4c3F)c12 ; C#Cc1cccc2cc(O)cc(-c3ccc4c(N5CC6CCC(C5)N6)nc(OCC5(CN6CCC7(C6)CC7(F)F)CC5)nc4c3F)c12 ; C#Cc1cccc2cc(O)cc(-c3ccc4c(N5CC6CCC(C5)N6)nc(OCC5(CN6C7CCC6C(F)(F)C7)CC5)nc4c3F)c12 ; C#Cc1cccc2cc(O)cc(-c3ccc4c(N5CC6CCC(C5)N6)nc(OCC5(CN6C7CCC6CC(F)C7)CC5)nc4c3F)c12]

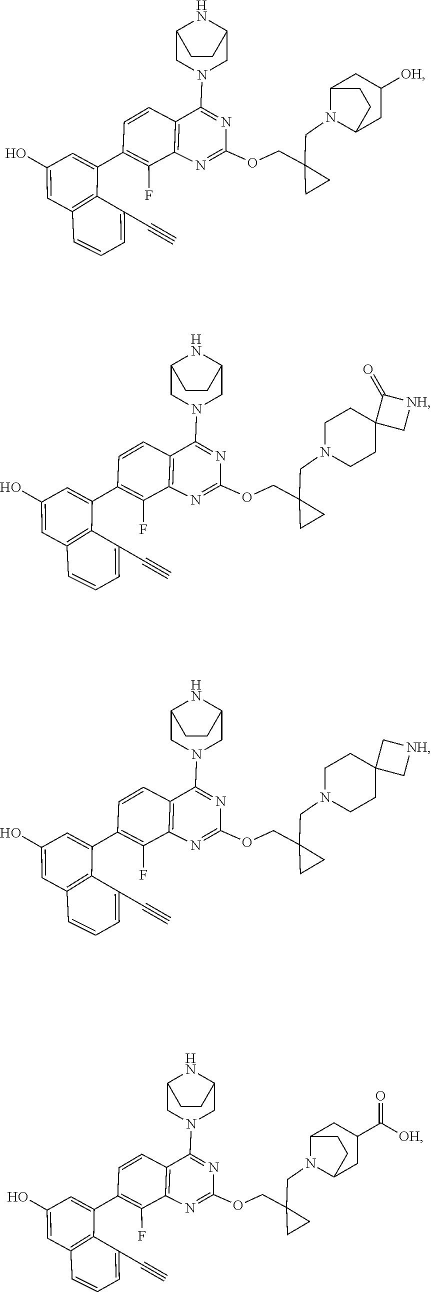 [SMILES: C#Cc1cccc2cc(O)cc(-c3ccc4c(N5CC6CCC(C5)N6)nc(OCC5(CN6C7CCC6CC(O)C7)CC5)nc4c3F)c12 ; C#Cc1cccc2cc(O)cc(-c3ccc4c(N5CC6CCC(C5)N6)nc(OCC5(CN6CCC7(CC6)CNC7=O)CC5)nc4c3F)c12 ; C#Cc1cccc2cc(O)cc(-c3ccc4c(N5CC6CCC(C5)N6)nc(OCC5(CN6CCC7(CC6)CNC7)CC5)nc4c3F)c12 ; C#Cc1cccc2cc(O)cc(-c3ccc4c(N5CC6CCC(C5)N6)nc(OCC5(CN6C7CCC6CC(C(=O)O)C7)CC5)nc4c3F)c12]

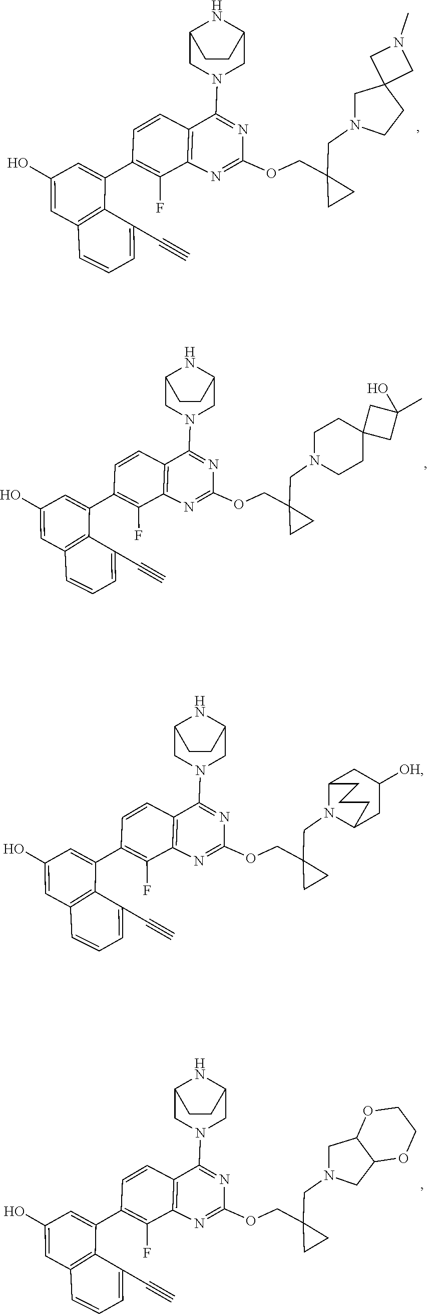 [SMILES: C#Cc1cccc2cc(O)cc(-c3ccc4c(N5CC6CCC(C5)N6)nc(OCC5(CN6CCC7(CN(C)C7)C6)CC5)nc4c3F)c12 ; C#Cc1cccc2cc(O)cc(-c3ccc4c(N5CC6CCC(C5)N6)nc(OCC5(CN6CCC7(CC6)CC(C)(O)C7)CC5)nc4c3F)c12 ; C#Cc1cccc2cc(O)cc(-c3ccc4c(N5CC6CCC(C5)N6)nc(OCC5(CN6C7CCCC6CC(O)C7)CC5)nc4c3F)c12 ; C#Cc1cccc2cc(O)cc(-c3ccc4c(N5CC6CCC(C5)N6)nc(OCC5(CN6CC7OCCOC7C6)CC5)nc4c3F)c12]

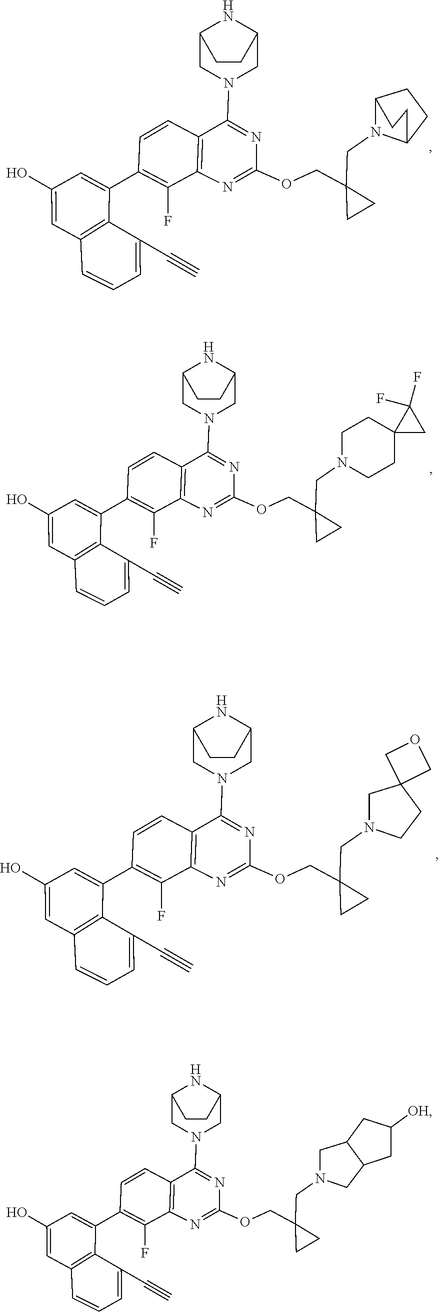 [SMILES: C#Cc1cccc2cc(O)cc(-c3ccc4c(N5CC6CCC(C5)N6)nc(OCC5(CN6C7CCC6CC7)CC5)nc4c3F)c12 ; C#Cc1cccc2cc(O)cc(-c3ccc4c(N5CC6CCC(C5)N6)nc(OCC5(CN6CCC7(CC6)CC7(F)F)CC5)nc4c3F)c12 ; C#Cc1cccc2cc(O)cc(-c3ccc4c(N5CC6CCC(C5)N6)nc(OCC5(CN6CCC7(COC7)C6)CC5)nc4c3F)c12 ; C#Cc1cccc2cc(O)cc(-c3ccc4c(N5CC6CCC(C5)N6)nc(OCC5(CN6CC7CC(O)CC7C6)CC5)nc4c3F)c12]

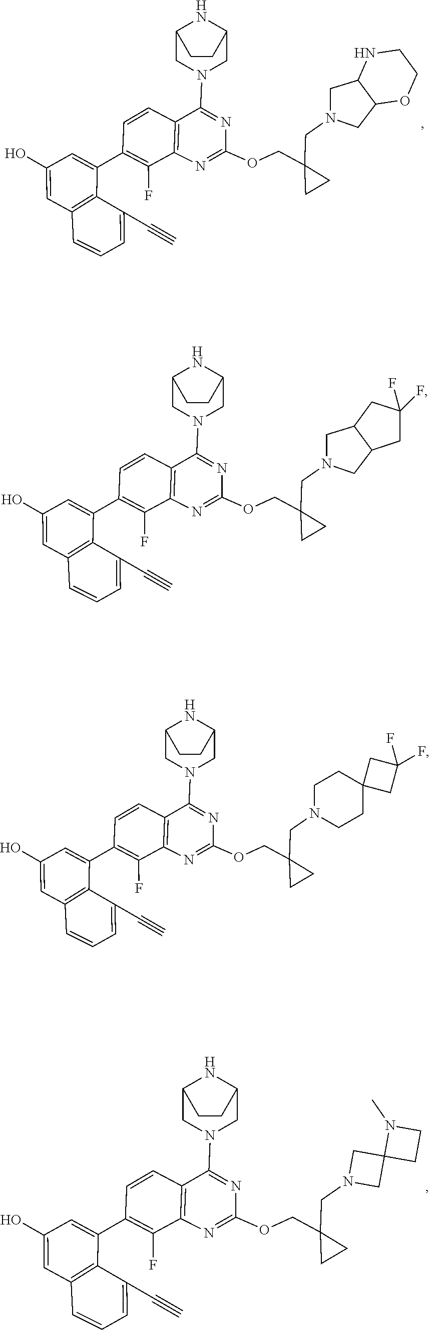 [SMILES: C#Cc1cccc2cc(O)cc(-c3ccc4c(N5CC6CCC(C5)N6)nc(OCC5(CN6CC7NCCOC7C6)CC5)nc4c3F)c12 ; C#Cc1cccc2cc(O)cc(-c3ccc4c(N5CC6CCC(C5)N6)nc(OCC5(CN6CC7CC(F)(F)CC7C6)CC5)nc4c3F)c12 ; C#Cc1cccc2cc(O)cc(-c3ccc4c(N5CC6CCC(C5)N6)nc(OCC5(CN6CCC7(CC6)CC(F)(F)C7)CC5)nc4c3F)c12 ; C#Cc1cccc2cc(O)cc(-c3ccc4c(N5CC6CCC(C5)N6)nc(OCC5(CN6CC7(CCN7C)C6)CC5)nc4c3F)c12]

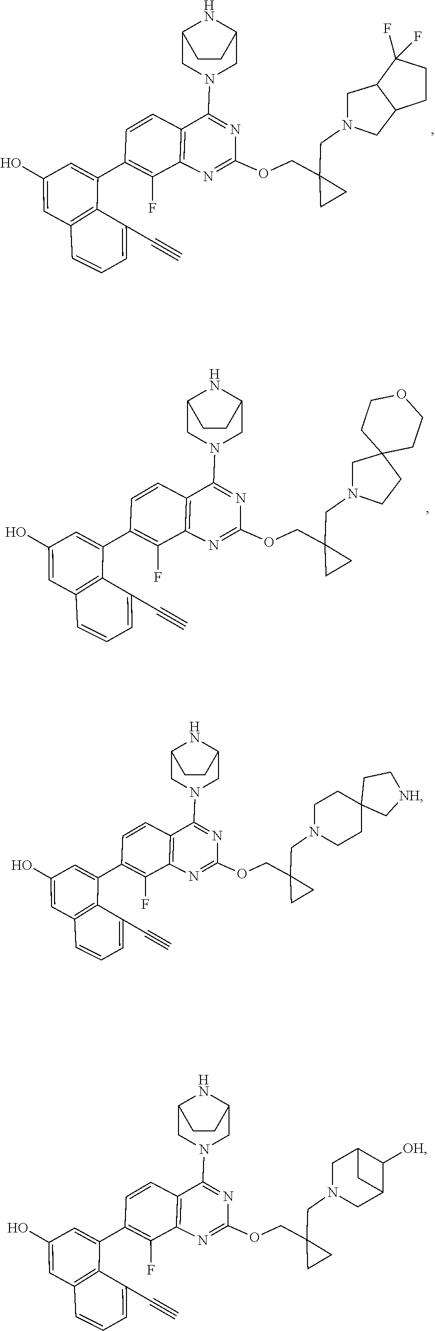 [SMILES: C#Cc1cccc2cc(O)cc(-c3ccc4c(N5CC6CCC(C5)N6)nc(OCC5(CN6CC7CCC(F)(F)C7C6)CC5)nc4c3F)c12 ; C#Cc1cccc2cc(O)cc(-c3ccc4c(N5CC6CCC(C5)N6)nc(OCC5(CN6CCC7(CCOCC7)C6)CC5)nc4c3F)c12 ; C#Cc1cccc2cc(O)cc(-c3ccc4c(N5CC6CCC(C5)N6)nc(OCC5(CN6CCC7(CCNC7)CC6)CC5)nc4c3F)c12 ; C#Cc1cccc2cc(O)cc(-c3ccc4c(N5CC6CCC(C5)N6)nc(OCC5(CN6CC7CC(C6)C7O)CC5)nc4c3F)c12]

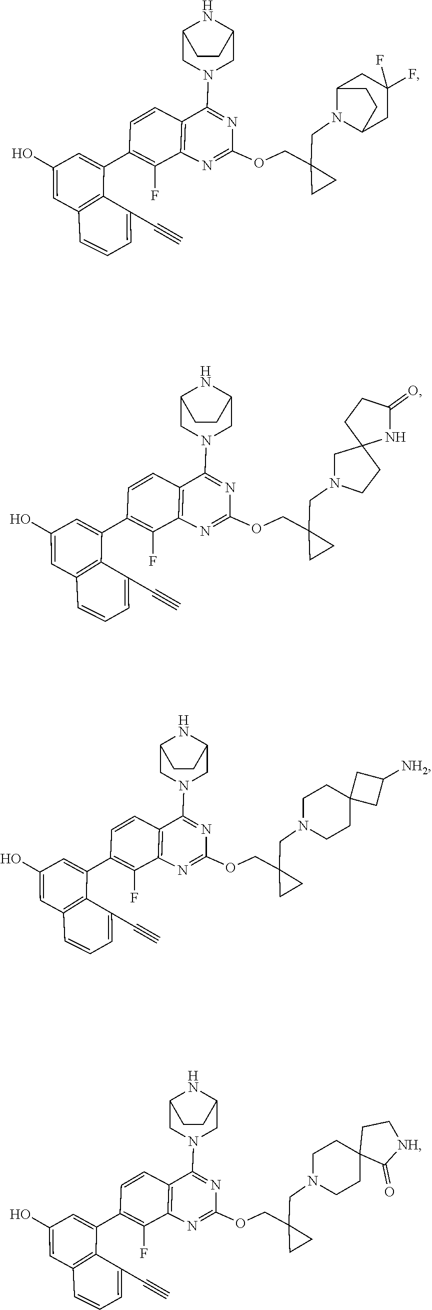 [SMILES: C#Cc1cccc2cc(O)cc(-c3ccc4c(N5CC6CCC(C5)N6)nc(OCC5(CN6C7CCC6CC(F)(F)C7)CC5)nc4c3F)c12 ; C#Cc1cccc2cc(O)cc(-c3ccc4c(N5CC6CCC(C5)N6)nc(OCC5(CN6CCC7(CCC(=O)N7)C6)CC5)nc4c3F)c12 ; C#Cc1cccc2cc(O)cc(-c3ccc4c(N5CC6CCC(C5)N6)nc(OCC5(CN6CCC7(CC6)CC(N)C7)CC5)nc4c3F)c12 ; C#Cc1cccc2cc(O)cc(-c3ccc4c(N5CC6CCC(C5)N6)nc(OCC5(CN6CCC7(CCNC7=O)CC6)CC5)nc4c3F)c12]

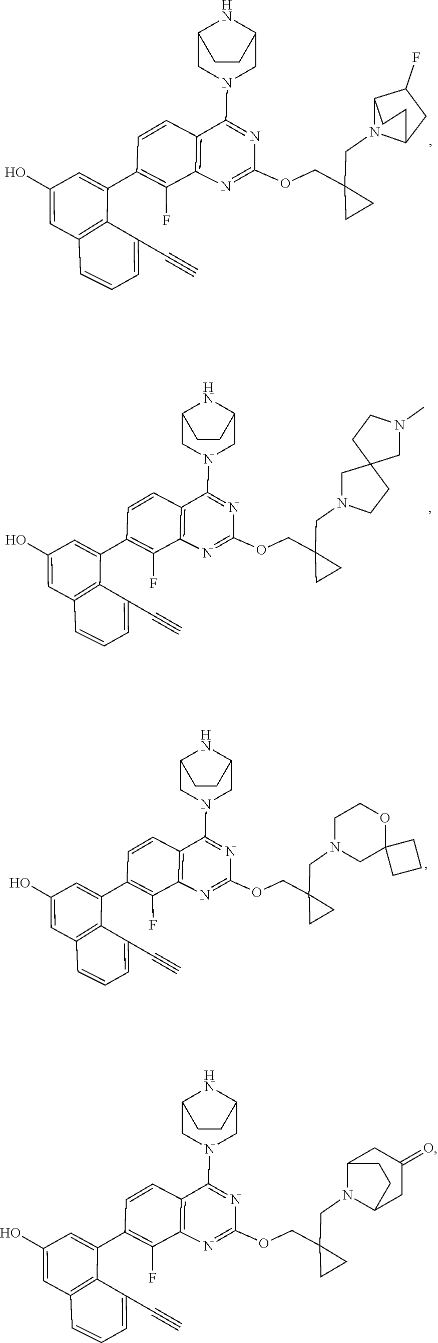 [SMILES: C#Cc1cccc2cc(O)cc(-c3ccc4c(N5CC6CCC(C5)N6)nc(OCC5(CN6C7CCC6C(F)C7)CC5)nc4c3F)c12 ; C#Cc1cccc2cc(O)cc(-c3ccc4c(N5CC6CCC(C5)N6)nc(OCC5(CN6CCC7(CCN(C)C7)C6)CC5)nc4c3F)c12 ; C#Cc1cccc2cc(O)cc(-c3ccc4c(N5CC6CCC(C5)N6)nc(OCC5(CN6CCOC7(CCC7)C6)CC5)nc4c3F)c12 ; C#Cc1cccc2cc(O)cc(-c3ccc4c(N5CC6CCC(C5)N6)nc(OCC5(CN6C7CCC6CC(=O)C7)CC5)nc4c3F)c12]

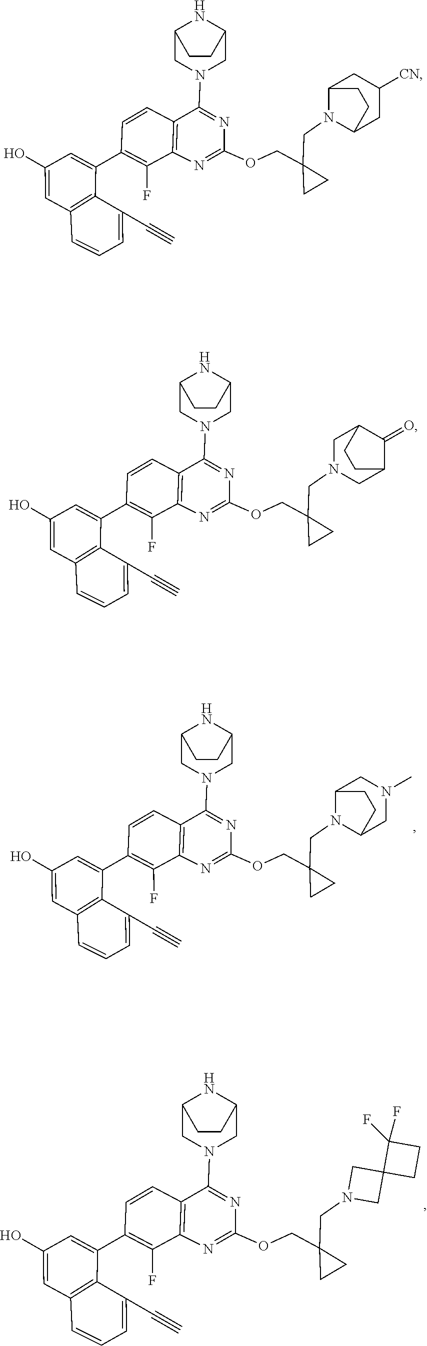 [SMILES: C#Cc1cccc2cc(O)cc(-c3ccc4c(N5CC6CCC(C5)N6)nc(OCC5(CN6C7CCC6CC(C#N)C7)CC5)nc4c3F)c12 ; C#Cc1cccc2cc(O)cc(-c3ccc4c(N5CC6CCC(C5)N6)nc(OCC5(CN6CC7CCC(C6)C7=O)CC5)nc4c3F)c12 ; C#Cc1cccc2cc(O)cc(-c3ccc4c(N5CC6CCC(C5)N6)nc(OCC5(CN6C7CCC6CN(C)C7)CC5)nc4c3F)c12 ; C#Cc1cccc2cc(O)cc(-c3ccc4c(N5CC6CCC(C5)N6)nc(OCC5(CN6CC7(CCC7(F)F)C6)CC5)nc4c3F)c12]

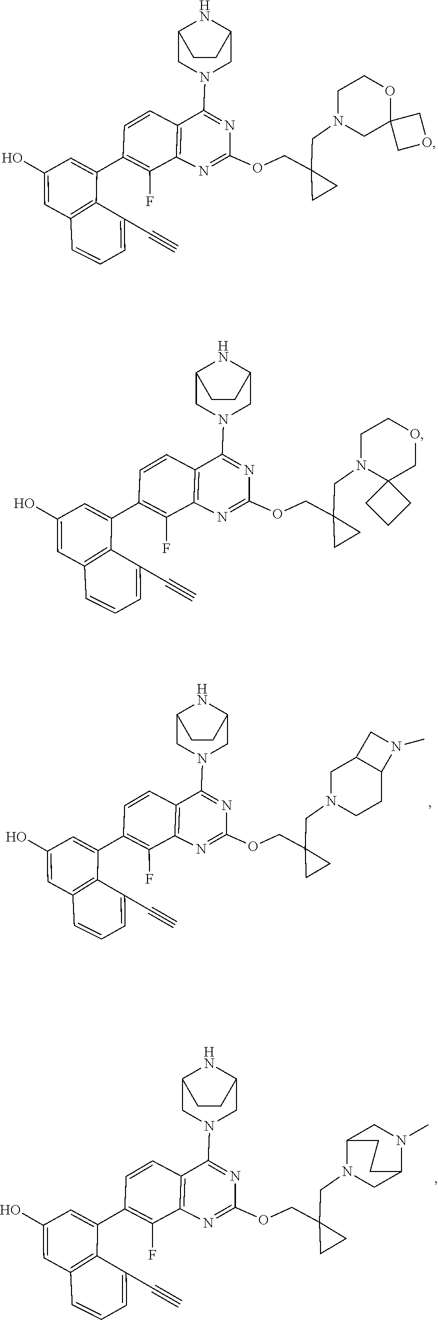 [SMILES: C#Cc1cccc2cc(O)cc(-c3ccc4c(N5CC6CCC(C5)N6)nc(OCC5(CN6CCOC7(COC7)C6)CC5)nc4c3F)c12 ; C#Cc1cccc2cc(O)cc(-c3ccc4c(N5CC6CCC(C5)N6)nc(OCC5(CN6CCOCC67CCC7)CC5)nc4c3F)c12 ; C#Cc1cccc2cc(O)cc(-c3ccc4c(N5CC6CCC(C5)N6)nc(OCC5(CN6CCC7C(C6)CN7C)CC5)nc4c3F)c12 ; C#Cc1cccc2cc(O)cc(-c3ccc4c(N5CC6CCC(C5)N6)nc(OCC5(CN6CC7CCC6CN7C)CC5)nc4c3F)c12]

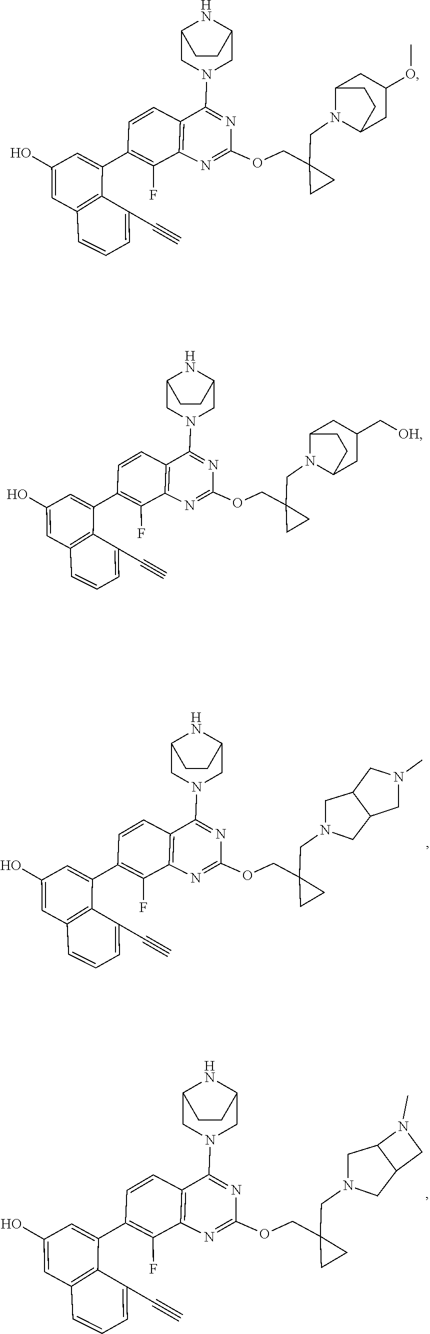 [SMILES: C#Cc1cccc2cc(O)cc(-c3ccc4c(N5CC6CCC(C5)N6)nc(OCC5(CN6C7CCC6CC(OC)C7)CC5)nc4c3F)c12 ; C#Cc1cccc2cc(O)cc(-c3ccc4c(N5CC6CCC(C5)N6)nc(OCC5(CN6C7CCC6CC(CO)C7)CC5)nc4c3F)c12 ; C#Cc1cccc2cc(O)cc(-c3ccc4c(N5CC6CCC(C5)N6)nc(OCC5(CN6CC7CN(C)CC7C6)CC5)nc4c3F)c12 ; C#Cc1cccc2cc(O)cc(-c3ccc4c(N5CC6CCC(C5)N6)nc(OCC5(CN6CC7CN(C)C7C6)CC5)nc4c3F)c12]

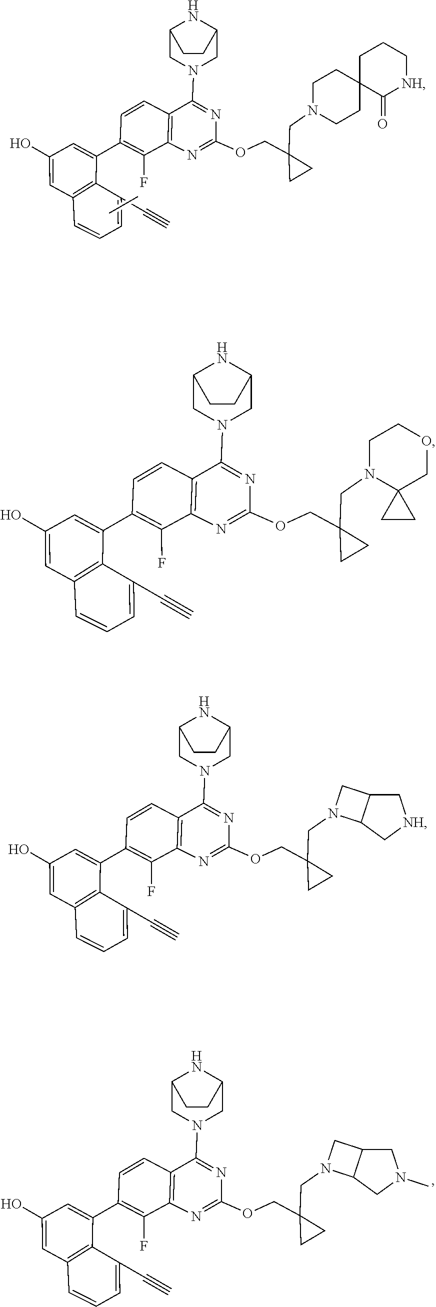

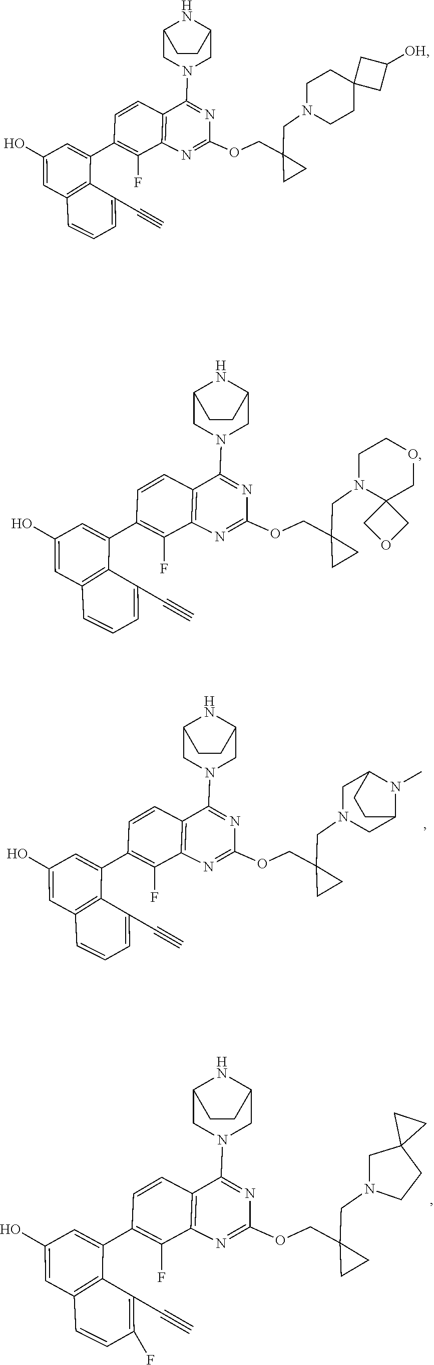 [SMILES: C#Cc1cccc2cc(O)cc(-c3ccc4c(N5CC6CCC(C5)N6)nc(OCC5(CN6CCC7(CC6)CC(O)C7)CC5)nc4c3F)c12 ; C#Cc1cccc2cc(O)cc(-c3ccc4c(N5CC6CCC(C5)N6)nc(OCC5(CN6CCOCC67COC7)CC5)nc4c3F)c12 ; C#Cc1cccc2cc(O)cc(-c3ccc4c(N5CC6CCC(C5)N6)nc(OCC5(CN6CC7CCC(C6)N7C)CC5)nc4c3F)c12 ; C#Cc1c(F)ccc2cc(O)cc(-c3ccc4c(N5CC6CCC(C5)N6)nc(OCC5(CN6CCC7(CC7)C6)CC5)nc4c3F)c12]

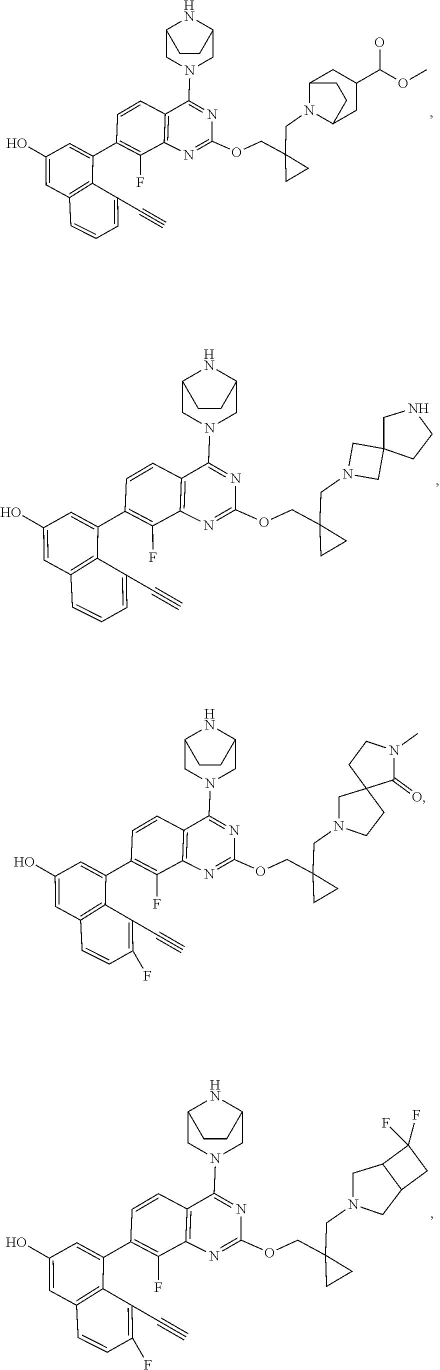 [SMILES: C#Cc1cccc2cc(O)cc(-c3ccc4c(N5CC6CCC(C5)N6)nc(OCC5(CN6C7CCC6CC(C(O)OC)C7)CC5)nc4c3F)c12 ; C#Cc1cccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CCC7(CCN(C)C7=O)C6)CC5)nc4c3F)c12 ; C#Cc1c(F)ccc2cc(O)cc(-c3ccc4c(N5CC6CCC(C5)N6)nc(OCC5(CN6CC7CC(F)(F)C7C6)CC5)nc4c3F)c12]

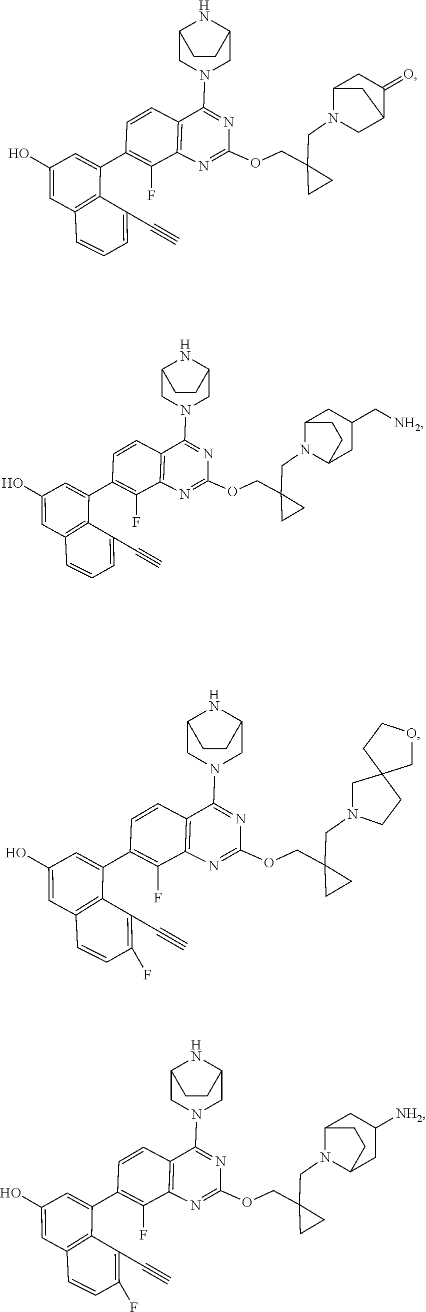 [SMILES: C#Cc1cccc2cc(O)cc(-c3ccc4c(N5CC6CCC(C5)N6)nc(OCC5(CN6CC7CC6CC7=O)CC5)nc4c3F)c12 ; C#Cc1cccc2cc(O)cc(-c3ccc4c(N5CC6CCC(C5)N6)nc(OCC5(CN6C7CCC6CC(CN)C7)CC5)nc4c3F)c12 ; C#Cc1c(F)ccc2cc(O)cc(-c3ccc4c(N5CC6CCC(C5)N6)nc(OCC5(CN6CCC7(CCOC7)C6)CC5)nc4c3F)c12 ; C#Cc1c(F)ccc2cc(O)cc(-c3ccc4c(N5CC6CCC(C5)N6)nc(OCC5(CN6C7CCC6CC(N)C7)CC5)nc4c3F)c12]

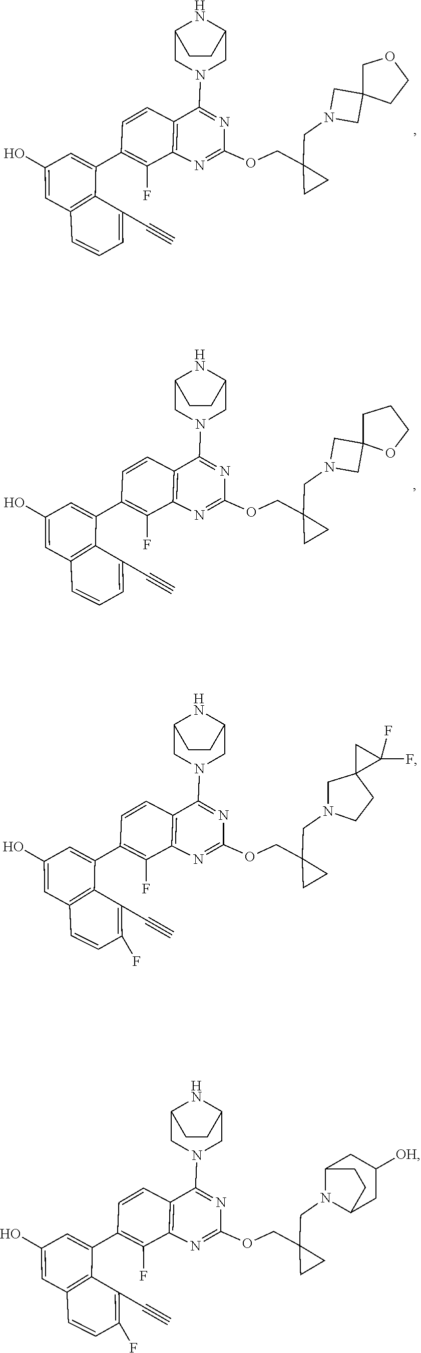 [SMILES: C#Cc1cccc2cc(O)cc(-c3ccc4c(N5CC6CCC(C5)N6)nc(OCC5(CN6CC7(CCOC7)C6)CC5)nc4c3F)c12 ; C#Cc1cccc2cc(O)cc(-c3ccc4c(N5CC6CCC(C5)N6)nc(OCC5(CN6CC7(CCCO7)C6)CC5)nc4c3F)c12 ; C#Cc1c(F)ccc2cc(O)cc(-c3ccc4c(N5CC6CCC(C5)N6)nc(OCC5(CN6CCC7(C6)CC7(F)F)CC5)nc4c3F)c12 ; C#Cc1c(F)ccc2cc(O)cc(-c3ccc4c(N5CC6CCC(C5)N6)nc(OCC5(CN6C7CCC6CC(O)C7)CC5)nc4c3F)c12]

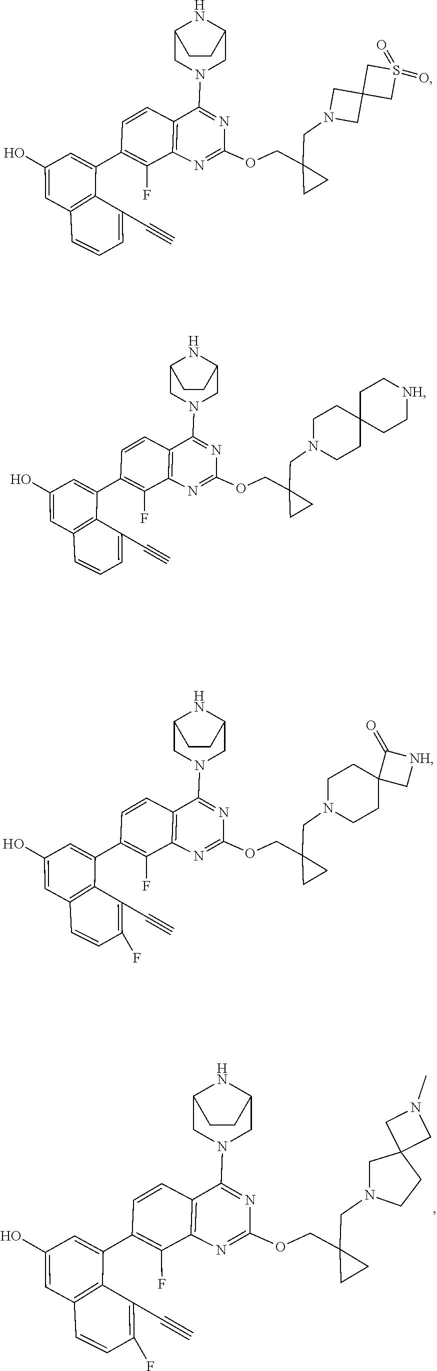 [SMILES: C#Cc1cccc2cc(O)cc(-c3ccc4c(N5CC6CCC(C5)N6)nc(OCC5(CN6CC7(C6)CS(=O)(=O)C7)CC5)nc4c3F)c12 ; C#Cc1cccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CCC7(CC6)CNC7=O)CC5)nc4c3F)c12 ; C#Cc1c(F)ccc2cc(O)cc(-c3ccc4c(N5CC6CCC(C5)N6)nc(OCC5(CN6CCC7(CN(C)C7)C6)CC5)nc4c3F)c12]

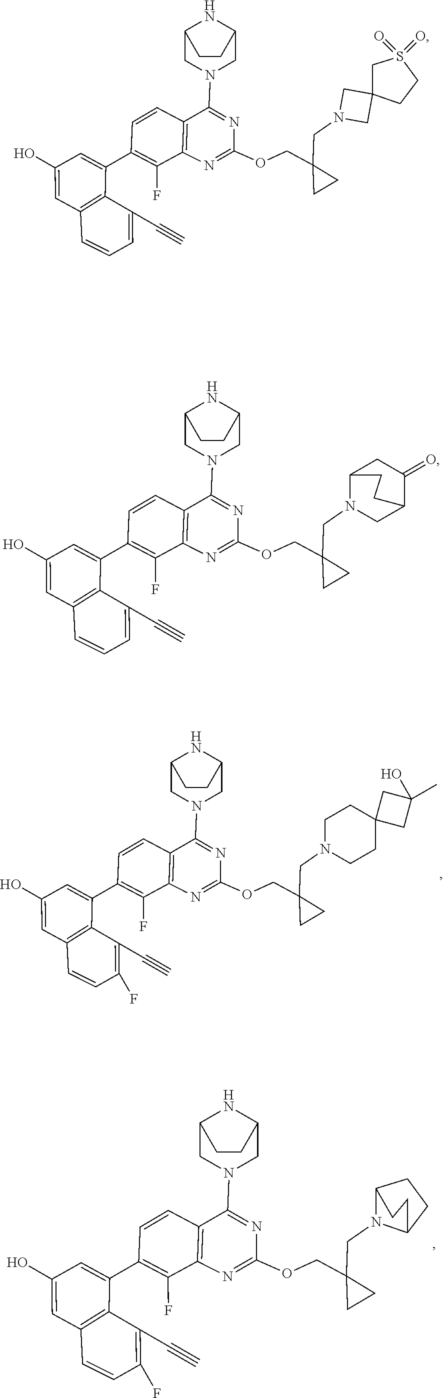 [SMILES: C#Cc1cccc2cc(O)cc(-c3ccc4c(N5CC6CCC(C5)N6)nc(OCC5(CN6CC7(CCS(=O)(=O)C7)C6)CC5)nc4c3F)c12 ; C#Cc1cccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6CCC7(CC6)CC(C)(O)C7)CC5)nc4c3F)c12 ; C#Cc1c(F)ccc2cc(O)cc(-c3ccc4c(N5CC6CCC(C5)N6)nc(OCC5(CN6C7CCC6CC7)CC5)nc4c3F)c12]

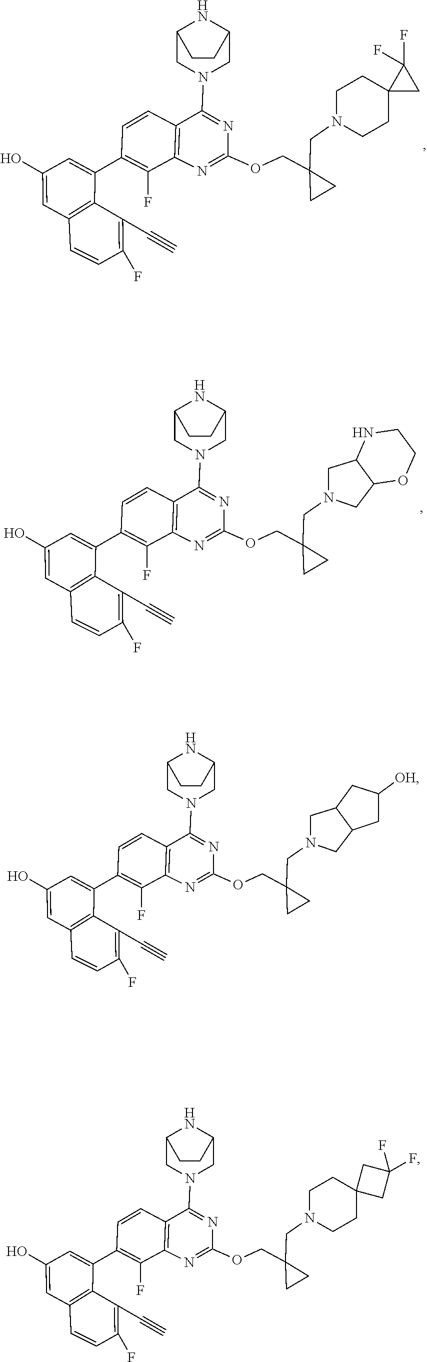 [SMILES: C#Cc1c(F)ccc2cc(O)cc(-c3ccc4c(N5CC6CCC(C5)N6)nc(OCC5(CN6CCC7(CC6)CC7(F)F)CC5)nc4c3F)c12 ; C#Cc1c(F)ccc2cc(O)cc(-c3ccc4c(N5CC6CCC(C5)N6)nc(OCC5(CN6CC7NCCOC7C6)CC5)nc4c3F)c12 ; C#Cc1c(F)ccc2cc(O)cc(-c3ccc4c(N5CC6CCC(C5)N6)nc(OCC5(CN6CC7CC(O)CC7C6)CC5)nc4c3F)c12 ; C#Cc1c(F)ccc2cc(O)cc(-c3ccc4c(N5CC6CCC(C5)N6)nc(OCC5(CN6CCC7(CC6)CC(F)(F)C7)CC5)nc4c3F)c12]

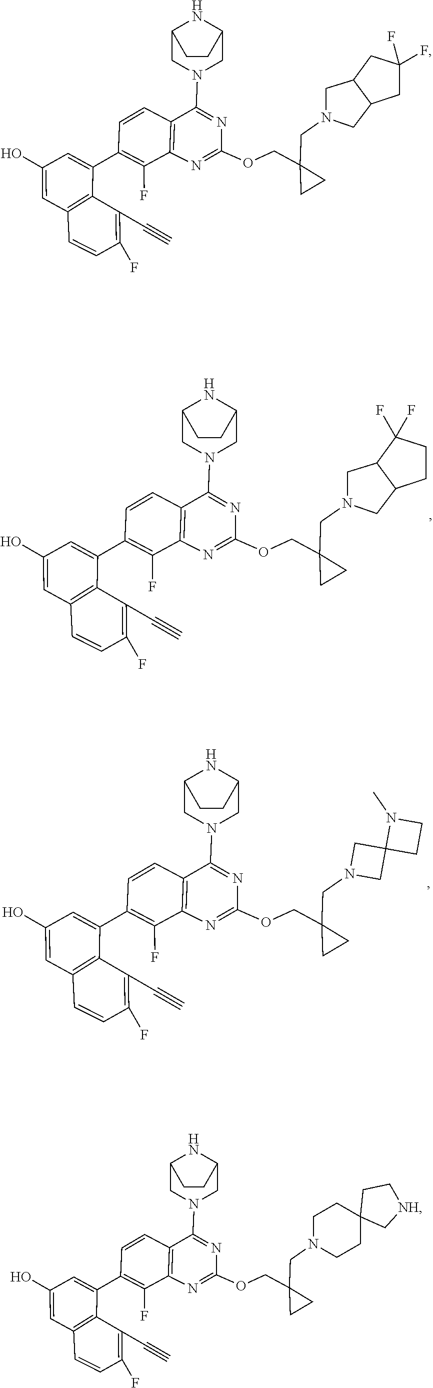 [SMILES: C#Cc1c(F)ccc2cc(O)cc(-c3ccc4c(N5CC6CCC(C5)N6)nc(OCC5(CN6CC7CC(F)(F)CC7C6)CC5)nc4c3F)c12 ; C#Cc1c(F)ccc2cc(O)cc(-c3ccc4c(N5CC6CCC(C5)N6)nc(OCC5(CN6CC7CCC(F)(F)C7C6)CC5)nc4c3F)c12 ; C#Cc1c(F)ccc2cc(O)cc(-c3ccc4c(N5CC6CCC(C5)N6)nc(OCC5(CN6CC7(CCN7C)C6)CC5)nc4c3F)c12 ; C#Cc1c(F)ccc2cc(O)cc(-c3ccc4c(N5CC6CCC(C5)N6)nc(OCC5(CN6CCC7(CCNC7)CC6)CC5)nc4c3F)c12]

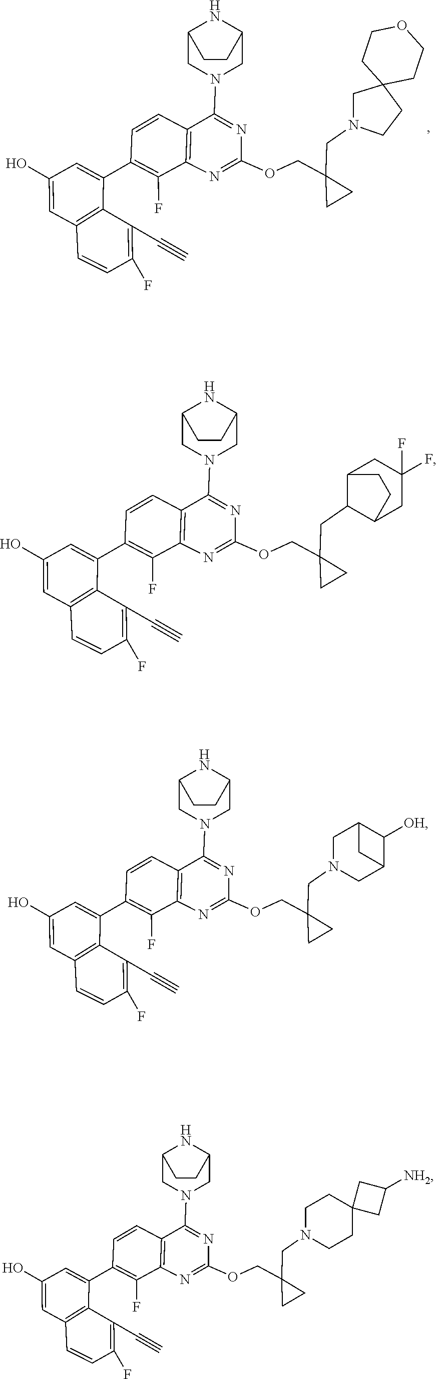 [SMILES: C#Cc1c(F)ccc2cc(O)cc(-c3ccc4c(N5CC6CCC(C5)N6)nc(OCC5(CN6CCC7(CCOCC7)C6)CC5)nc4c3F)c12 ; C#Cc1c(F)ccc2cc(O)cc(-c3ccc4c(N5CC6CCC(C5)N6)nc(OCC5(CC6C7CCC6CC(F)(F)C7)CC5)nc4c3F)c12 ; C#Cc1c(F)ccc2cc(O)cc(-c3ccc4c(N5CC6CCC(C5)N6)nc(OCC5(CN6CC7CC(C6)C7O)CC5)nc4c3F)c12 ; C#Cc1c(F)ccc2cc(O)cc(-c3ccc4c(N5CC6CCC(C5)N6)nc(OCC5(CN6CCC7(CC6)CC(N)C7)CC5)nc4c3F)c12]

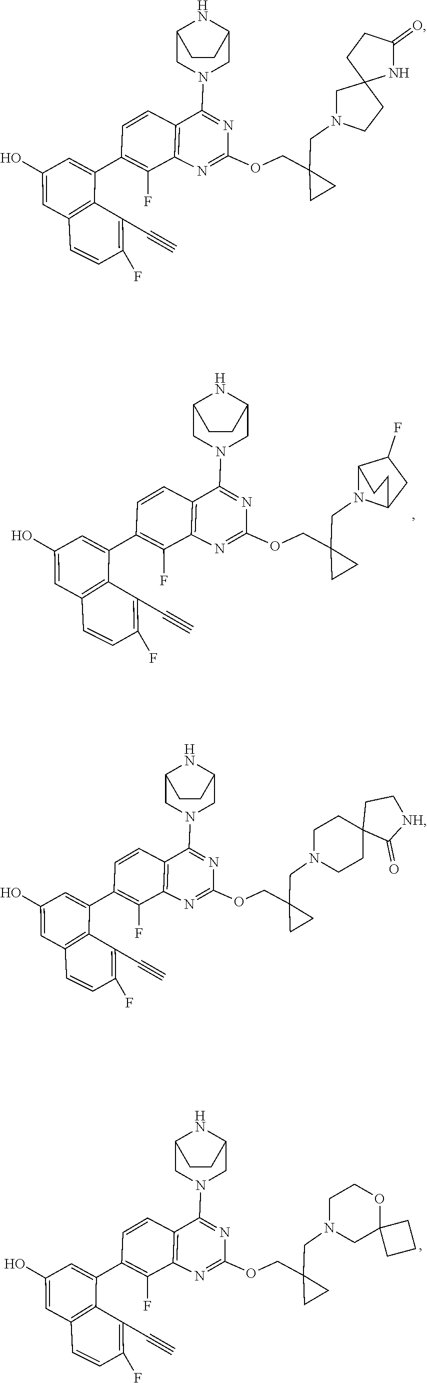 [SMILES: C#Cc1c(F)ccc2cc(O)cc(-c3ccc4c(N5CC6CCC(C5)N6)nc(OCC5(CN6CCC7(CCC(=O)N7)C6)CC5)nc4c3F)c12 ; C#Cc1c(F)ccc2cc(O)cc(-c3ccc4c(N5CC6CCC(C5)N6)nc(OCC5(CN6C7CCC6C(F)C7)CC5)nc4c3F)c12 ; C#Cc1c(F)ccc2cc(O)cc(-c3ccc4c(N5CC6CCC(C5)N6)nc(OCC5(CN6CCC7(CCNC7=O)CC6)CC5)nc4c3F)c12 ; C#Cc1c(F)ccc2cc(O)cc(-c3ccc4c(N5CC6CCC(C5)N6)nc(OCC5(CN6CCOC7(CCC7)C6)CC5)nc4c3F)c12]

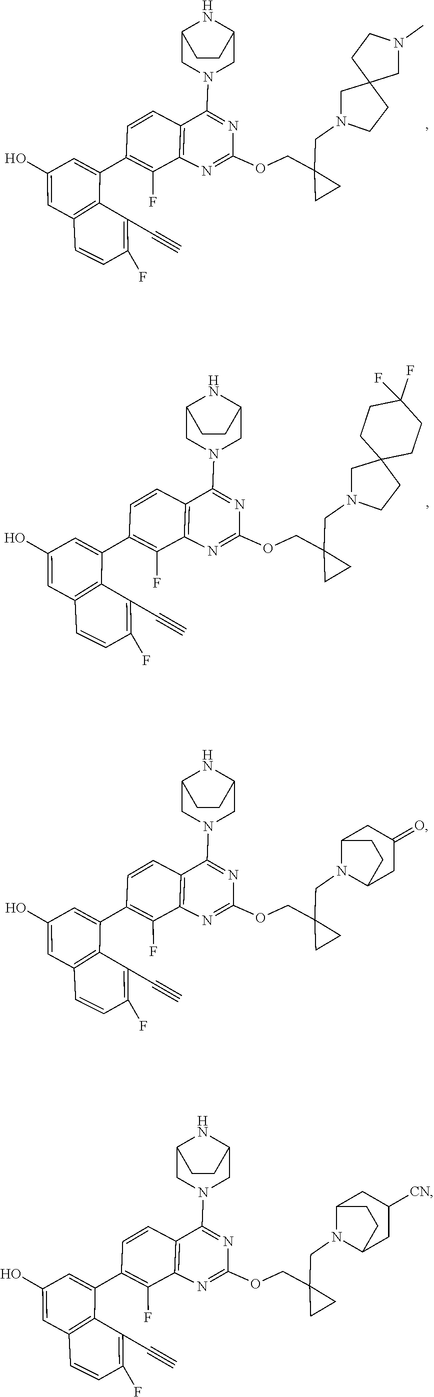 [SMILES: C#Cc1c(F)ccc2cc(O)cc(-c3ccc4c(N5CC6CCC(C5)N6)nc(OCC5(CN6CCC7(CCN(C)C7)C6)CC5)nc4c3F)c12 ; C#Cc1c(F)ccc2cc(O)cc(-c3ccc4c(N5CC6CCC(C5)N6)nc(OCC5(CN6CCC7(CCC(F)(F)CC7)C6)CC5)nc4c3F)c12 ; C#Cc1c(F)ccc2cc(O)cc(-c3ccc4c(N5CC6CCC(C5)N6)nc(OCC5(CN6C7CCC6CC(=O)C7)CC5)nc4c3F)c12 ; C#Cc1c(F)ccc2cc(O)cc(-c3ccc4c(N5CC6CCC(C5)N6)nc(OCC5(CN6C7CCC6CC(C#N)C7)CC5)nc4c3F)c12]

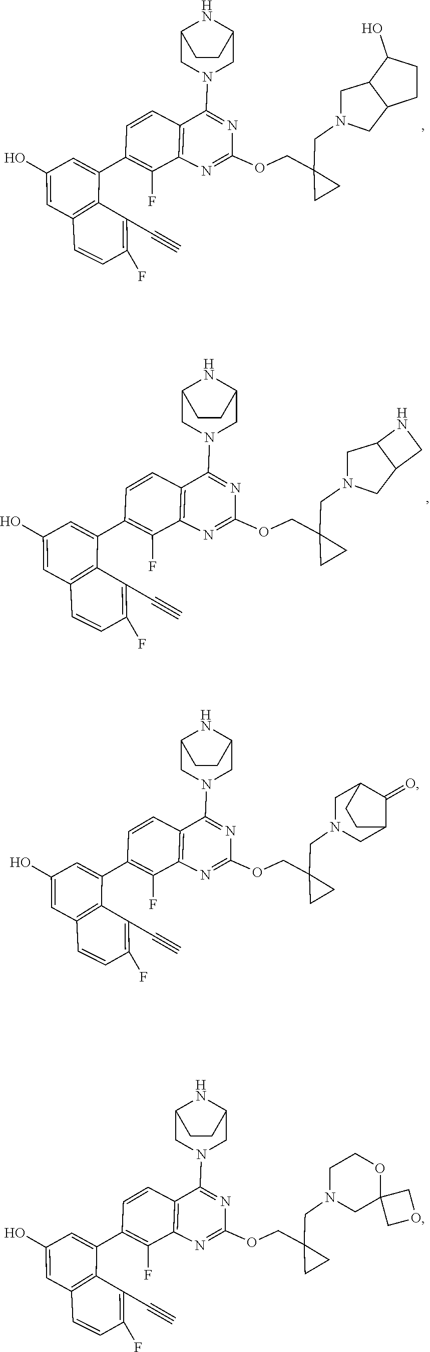 [SMILES: C#Cc1c(F)ccc2cc(O)cc(-c3ccc4c(N5CC6CCC(C5)N6)nc(OCC5(CN6CC7CCC(O)C7C6)CC5)nc4c3F)c12 ; C#Cc1c(F)ccc2cc(O)cc(-c3ccc4c(N5CC6CCC(C5)N6)nc(OCC5(CN6CC7CNC7C6)CC5)nc4c3F)c12 ; C#Cc1c(F)ccc2cc(O)cc(-c3ccc4c(N5CC6CCC(C5)N6)nc(OCC5(CN6CC7CCC(C6)C7=O)CC5)nc4c3F)c12 ; C#Cc1c(F)ccc2cc(O)cc(-c3ccc4c(N5CC6CCC(C5)N6)nc(OCC5(CN6CCOC7(COC7)C6)CC5)nc4c3F)c12]

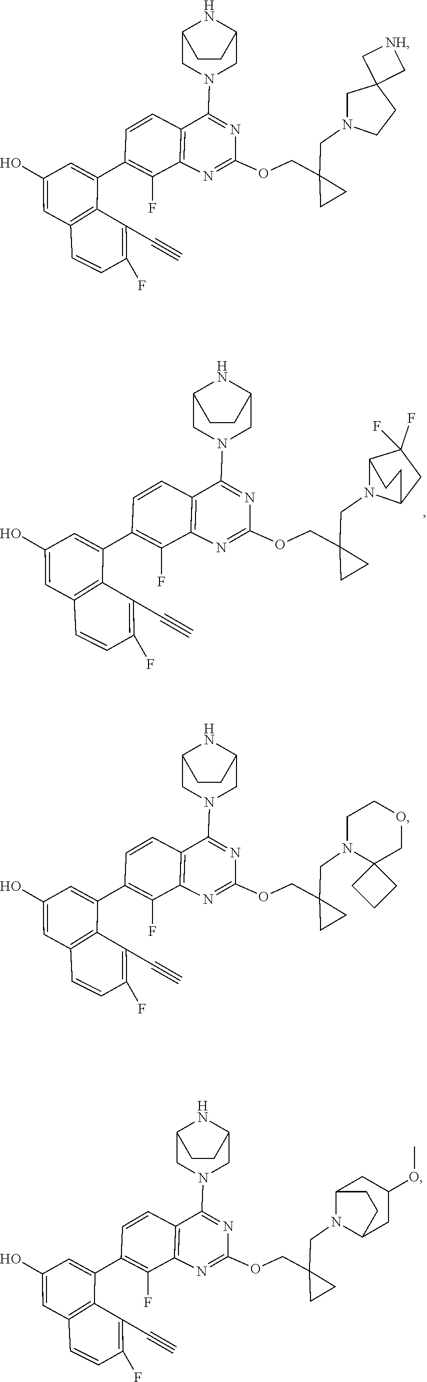 [SMILES: C#Cc1c(F)ccc2cc(O)cc(-c3ccc4c(N5CC6CCC(C5)N6)nc(OCC5(CN6CCC7(CNC7)C6)CC5)nc4c3F)c12 ; C#Cc1c(F)ccc2cc(O)cc(-c3ccc4c(N5CC6CCC(C5)N6)nc(OCC5(CN6C7CCC6C(F)(F)C7)CC5)nc4c3F)c12 ; C#Cc1c(F)ccc2cc(O)cc(-c3ccc4c(N5CC6CCC(C5)N6)nc(OCC5(CN6CCOCC67CCC7)CC5)nc4c3F)c12 ; C#Cc1c(F)ccc2cc(O)cc(-c3ccc4c(N5CC6CCC(C5)N6)nc(OCC5(CN6C7CCC6CC(OC)C7)CC5)nc4c3F)c12]

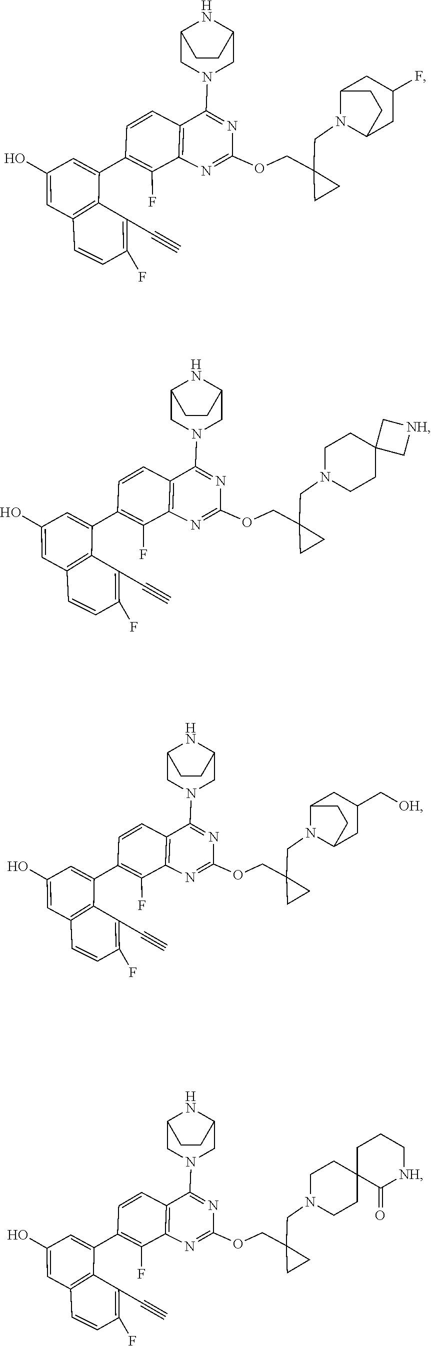 [SMILES: C#Cc1c(F)ccc2cc(O)cc(-c3ccc4c(N5CC6CCC(C5)N6)nc(OCC5(CN6C7CCC6CC(F)C7)CC5)nc4c3F)c12 ; C#Cc1c(F)ccc2cc(O)cc(-c3ccc4c(N5CC6CCC(C5)N6)nc(OCC5(CN6CCC7(CC6)CNC7)CC5)nc4c3F)c12 ; C#Cc1c(F)ccc2cc(O)cc(-c3ccc4c(N5CC6CCC(C5)N6)nc(OCC5(CN6C7CCC6CC(CO)C7)CC5)nc4c3F)c12 ; C#Cc1c(F)ccc2cc(O)cc(-c3ccc4c(N5CC6CCC(C5)N6)nc(OCC5(CN6CCC7(CCCNC7=O)CC6)CC5)nc4c3F)c12]

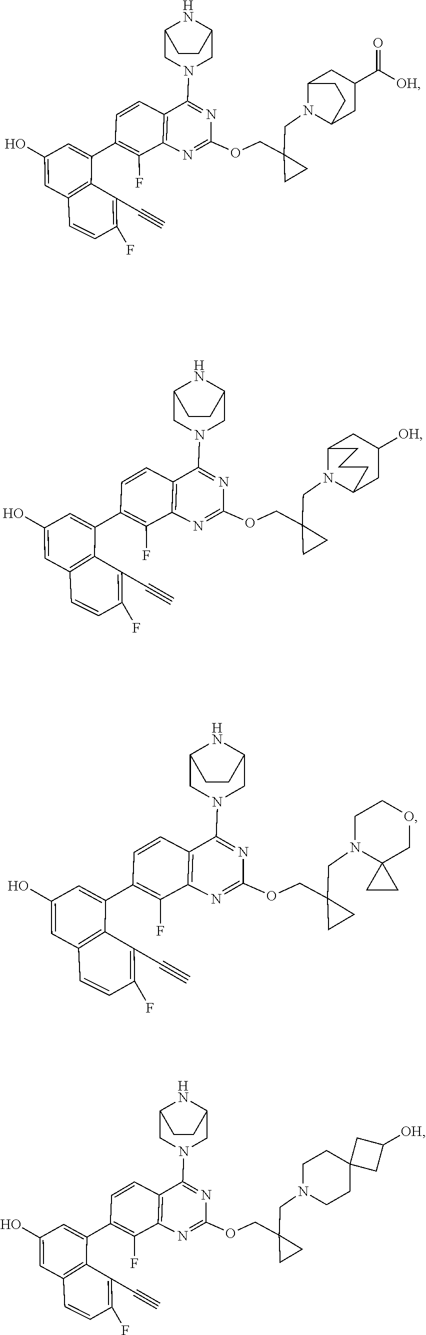 [SMILES: C#Cc1c(F)ccc2cc(O)cc(-c3ccc4c(N5CC6CCC(C5)N6)nc(OCC5(CN6C7CCC6CC(C(=O)O)C7)CC5)nc4c3F)c12 ; C#Cc1c(F)ccc2cc(O)cc(-c3ccc4c(N5CC6CCC(C5)N6)nc(OCC5(CN6C7CCCC6CC(O)C7)CC5)nc4c3F)c12 ; C#Cc1c(F)ccc2cc(O)cc(-c3ccc4c(N5CC6CCC(C5)N6)nc(OCC5(CN6CCOCC67CC7)CC5)nc4c3F)c12 ; C#Cc1c(F)ccc2cc(O)cc(-c3ccc4c(N5CC6CCC(C5)N6)nc(OCC5(CN6CCC7(CC6)CC(O)C7)CC5)nc4c3F)c12]

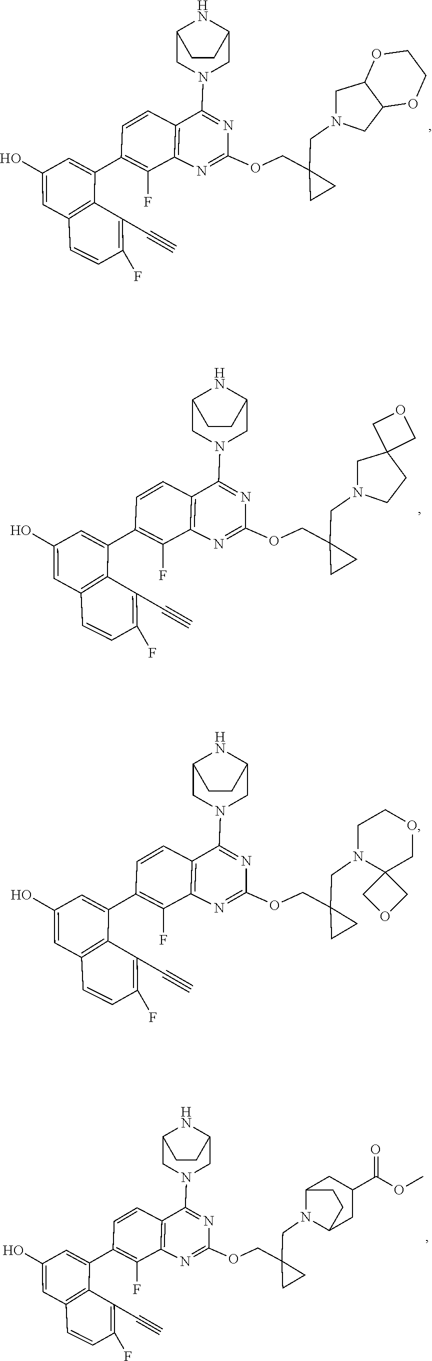 [SMILES: C#Cc1c(F)ccc2cc(O)cc(-c3ccc4c(N5CC6CCC(C5)N6)nc(OCC5(CN6CC7OCCOC7C6)CC5)nc4c3F)c12 ; C#Cc1c(F)ccc2cc(O)cc(-c3ccc4c(N5CC6CCC(C5)N6)nc(OCC5(CN6CCC7(COC7)C6)CC5)nc4c3F)c12 ; C#Cc1c(F)ccc2cc(O)cc(-c3ccc4c(N5CC6CCC(C5)N6)nc(OCC5(CN6CCOCC67COC7)CC5)nc4c3F)c12 ; C#Cc1c(F)ccc2cc(O)cc(-c3ccc4c(N5CC6CCC(C5)N6)nc(OCC5(CN6C7CCC6CC(C(=O)OC)C7)CC5)nc4c3F)c12]

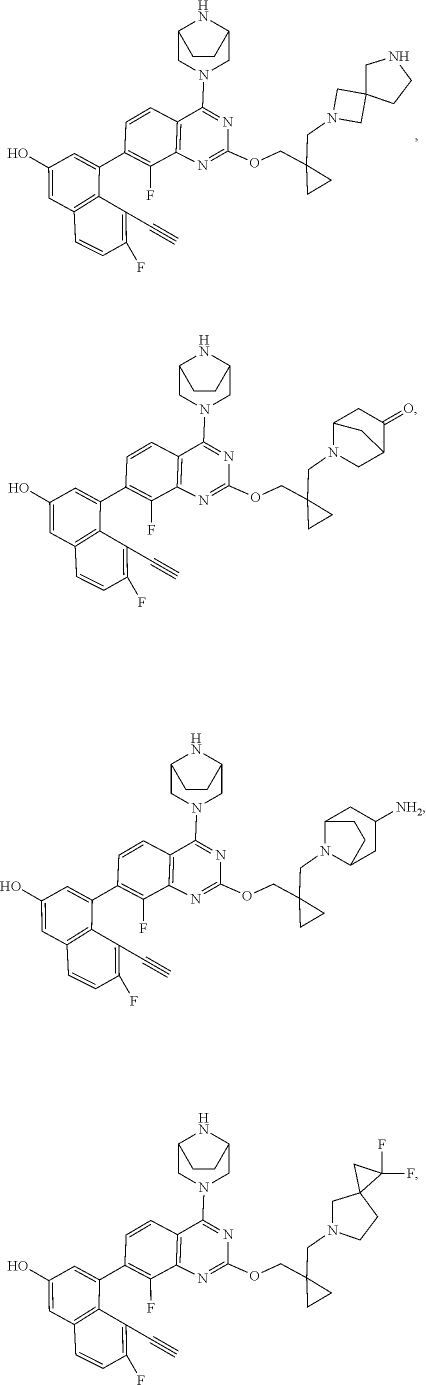 [SMILES: C#Cc1c(F)ccc2cc(O)cc(-c3ccc4c(N5CC6CCC(C5)N6)nc(OCC5(CN6CC7(CCNC7)C6)CC5)nc4c3F)c12 ; C#Cc1c(F)ccc2cc(O)cc(-c3ccc4c(N5CC6CCC(C5)N6)nc(OCC5(CN6CC7CC6CC7=O)CC5)nc4c3F)c12 ; C#Cc1c(F)ccc2cc(O)cc(-c3ccc4c(N5CC6CCC(C5)N6)nc(OCC5(CN6C7CCC6CC(N)C7)CC5)nc4c3F)c12 ; C#Cc1c(F)ccc2cc(O)cc(-c3ccc4c(N5CC6CCC(C5)N6)nc(OCC5(CN6CCC7(C6)CC7(F)F)CC5)nc4c3F)c12]

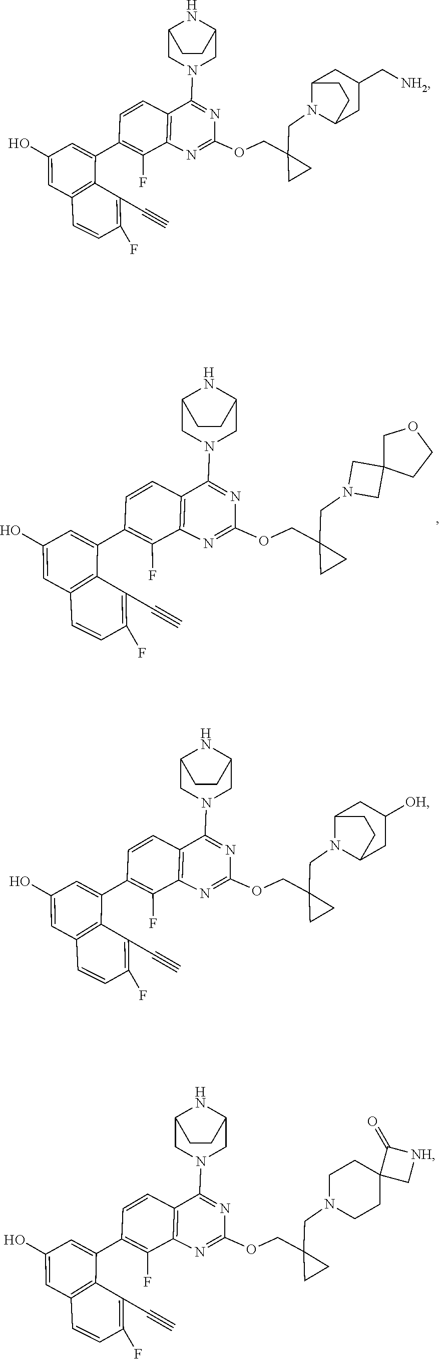 [SMILES: C#Cc1c(F)ccc2cc(O)cc(-c3ccc4c(N5CC6CCC(C5)N6)nc(OCC5(CN6C7CCC6CC(CN)C7)CC5)nc4c3F)c12 ; C#Cc1c(F)ccc2cc(O)cc(-c3ccc4c(N5CC6CCC(C5)N6)nc(OCC5(CN6CC7(CCOC7)C6)CC5)nc4c3F)c12 ; C#Cc1c(F)ccc2cc(O)cc(-c3ccc4c(N5CC6CCC(C5)N6)nc(OCC5(CN6C7CCC6CC(O)C7)CC5)nc4c3F)c12 ; C#Cc1c(F)ccc2cc(O)cc(-c3ccc4c(N5CC6CCC(C5)N6)nc(OCC5(CN6CCC7(CC6)CNC7=O)CC5)nc4c3F)c12]

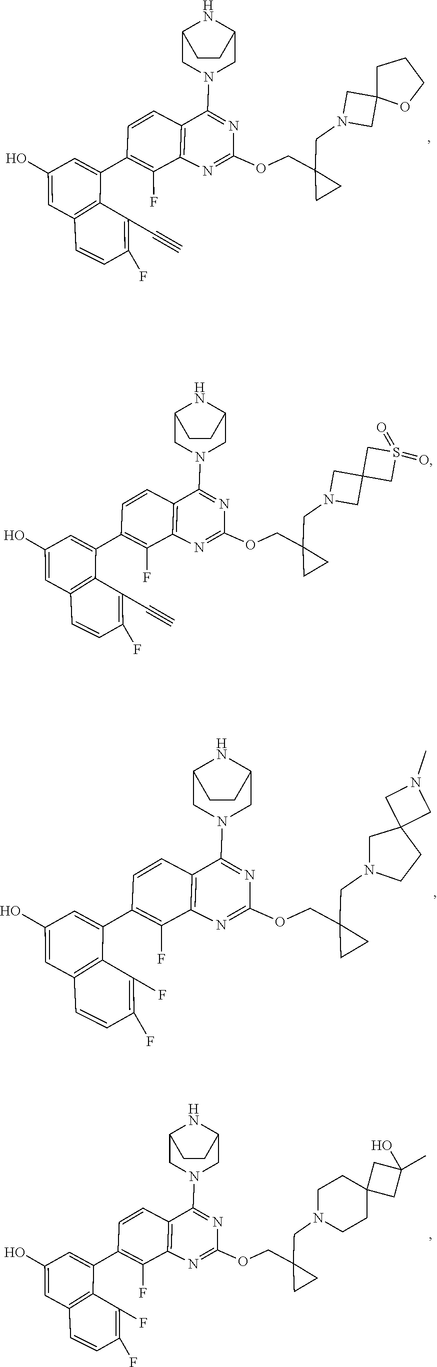 [SMILES: C#Cc1c(F)ccc2cc(O)cc(-c3ccc4c(N5CC6CCC(C5)N6)nc(OCC5(CN6CC7(CCCO7)C6)CC5)nc4c3F)c12 ; C#Cc1c(F)ccc2cc(O)cc(-c3ccc4c(N5CC6CCC(C5)N6)nc(OCC5(CN6CC7(C6)CS(=O)(=O)C7)CC5)nc4c3F)c12 ; CN1CC2(CCN(CC3(COc4nc(N5CC6CCC(C5)N6)c5ccc(-c6cc(O)cc7ccc(F)c(F)c67)c(F)c5n4)CC3)C2)C1 ; CC1(O)CC2(CCN(CC3(COc4nc(N5CC6CCC(C5)N6)c5ccc(-c6cc(O)cc7ccc(F)c(F)c67)c(F)c5n4)CC3)CC2)C1]

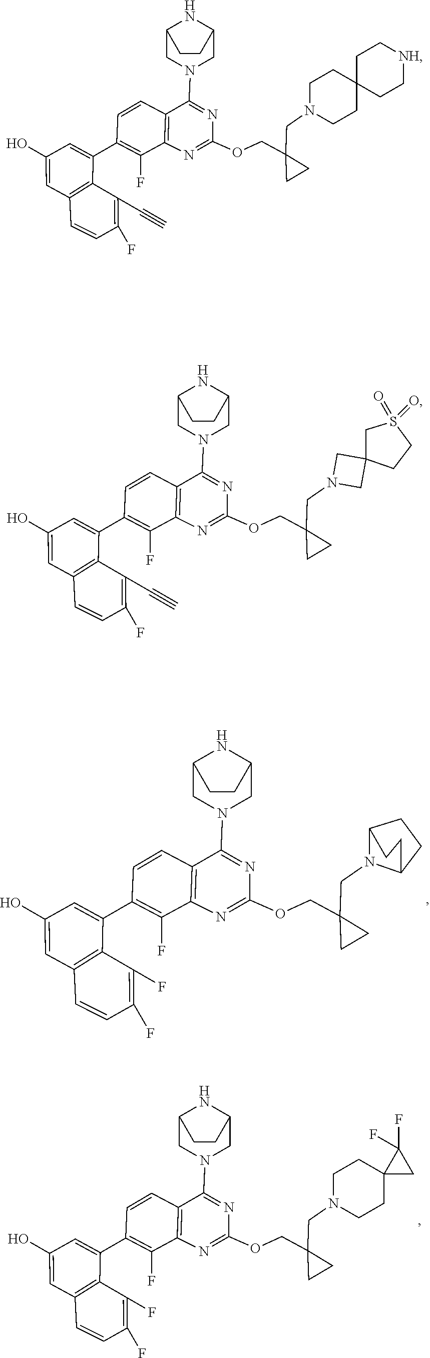 [SMILES: C#Cc1c(F)ccc2cc(O)cc(-c3ccc4c(N5CC6CCC(C5)N6)nc(OCC5(CN6CCC7(CCNCC7)CC6)CC5)nc4c3F)c12 ; C#Cc1c(F)ccc2cc(O)cc(-c3ccc4c(N5CC6CCC(C5)N6)nc(OCC5(CN6CC7(CCS(=O)(=O)C7)C6)CC5)nc4c3F)c12 ; Oc1cc(-c2ccc3c(N4CC5CCC(C4)N5)nc(OCC4(CN5C6CCC5CC6)CC4)nc3c2F)c2c(F)c(F)ccc2c1 ; Oc1cc(-c2ccc3c(N4CC5CCC(C4)N5)nc(OCC4(CN5CCC6(CC5)CC6(F)F)CC4)nc3c2F)c2c(F)c(F)ccc2c1]

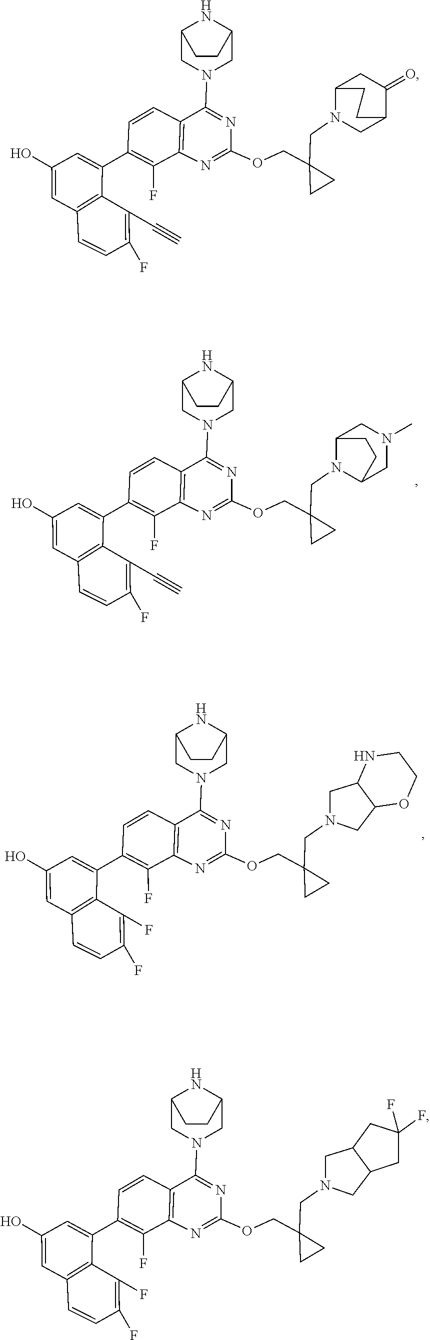 [SMILES: C#Cc1c(F)ccc2cc(O)cc(-c3ccc4c(N5CC6CCC(C5)N6)nc(OCC5(CN6CC7CCC6CC7=O)CC5)nc4c3F)c12 ; C#Cc1c(F)ccc2cc(O)cc(-c3ccc4c(N5CC6CCC(C5)N6)nc(OCC5(CN6C7CCC6CN(C)C7)CC5)nc4c3F)c12 ; Oc1cc(-c2ccc3c(N4CC5CCC(C4)N5)nc(OCC4(CN5CC6NCCOC6C5)CC4)nc3c2F)c2c(F)c(F)ccc2c1 ; Oc1cc(-c2ccc3c(N4CC5CCC(C4)N5)nc(OCC4(CN5CC6CC(F)(F)CC6C5)CC4)nc3c2F)c2c(F)c(F)ccc2c1]

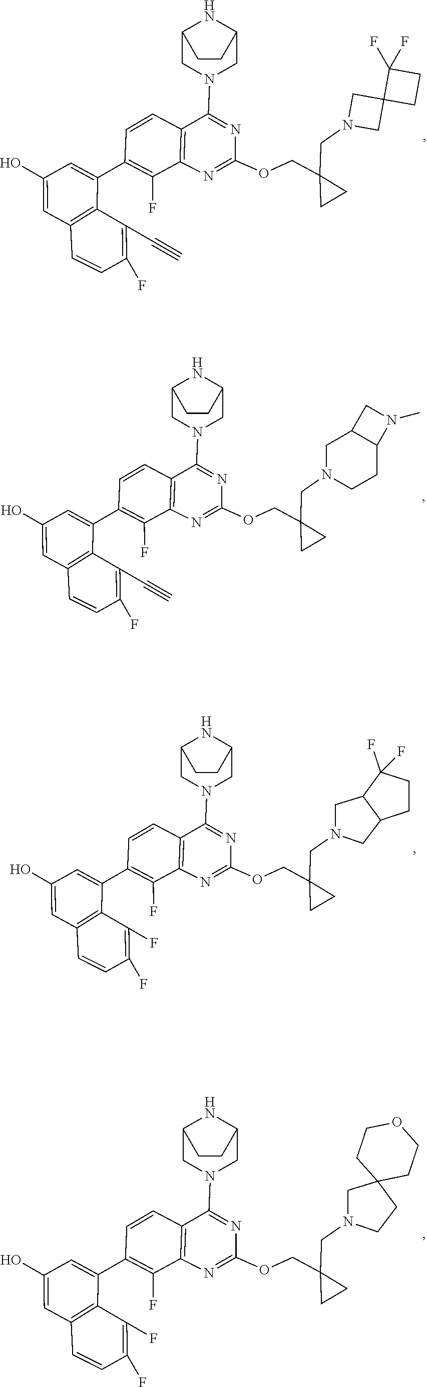 [SMILES: C#Cc1c(F)ccc2cc(O)cc(-c3ccc4c(N5CC6CCC(C5)N6)nc(OCC5(CN6CC7(CCC7(F)F)C6)CC5)nc4c3F)c12 ; C#Cc1c(F)ccc2cc(O)cc(-c3ccc4c(N5CC6CCC(C5)N6)nc(OCC5(CN6CCC7C(C6)CN7C)CC5)nc4c3F)c12 ; Oc1cc(-c2ccc3c(N4CC5CCC(C4)N5)nc(OCC4(CN5CC6CCC(F)(F)C6C5)CC4)nc3c2F)c2c(F)c(F)ccc2c1 ; Oc1cc(-c2ccc3c(N4CC5CCC(C4)N5)nc(OCC4(CN5CCC6(CCOCC6)C5)CC4)nc3c2F)c2c(F)c(F)ccc2c1]

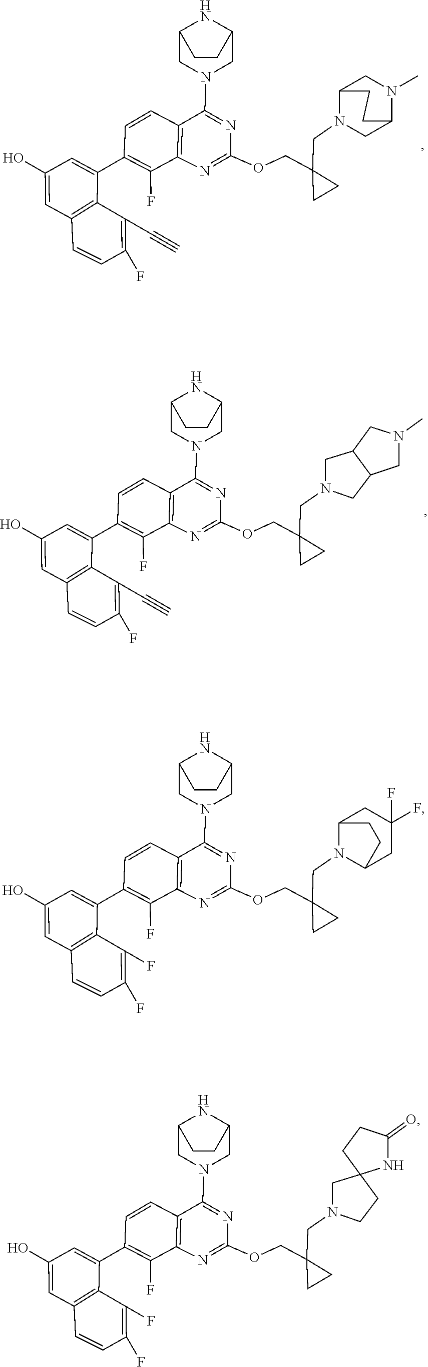 [SMILES: C#Cc1c(F)ccc2cc(O)cc(-c3ccc4c(N5CC6CCC(C5)N6)nc(OCC5(CN6CC7CCC6CN7C)CC5)nc4c3F)c12 ; C#Cc1c(F)ccc2cc(O)cc(-c3ccc4c(N5CC6CCC(C5)N6)nc(OCC5(CN6CC7CN(C)CC7C6)CC5)nc4c3F)c12 ; Oc1cc(-c2ccc3c(N4CC5CCC(C4)N5)nc(OCC4(CN5C6CCC5CC(F)(F)C6)CC4)nc3c2F)c2c(F)c(F)ccc2c1 ; O=C1CCC2(CCN(CC3(COc4nc(N5CC6CCC(C5)N6)c5ccc(-c6cc(O)cc7ccc(F)c(F)c67)c(F)c5n4)CC3)C2)N1]

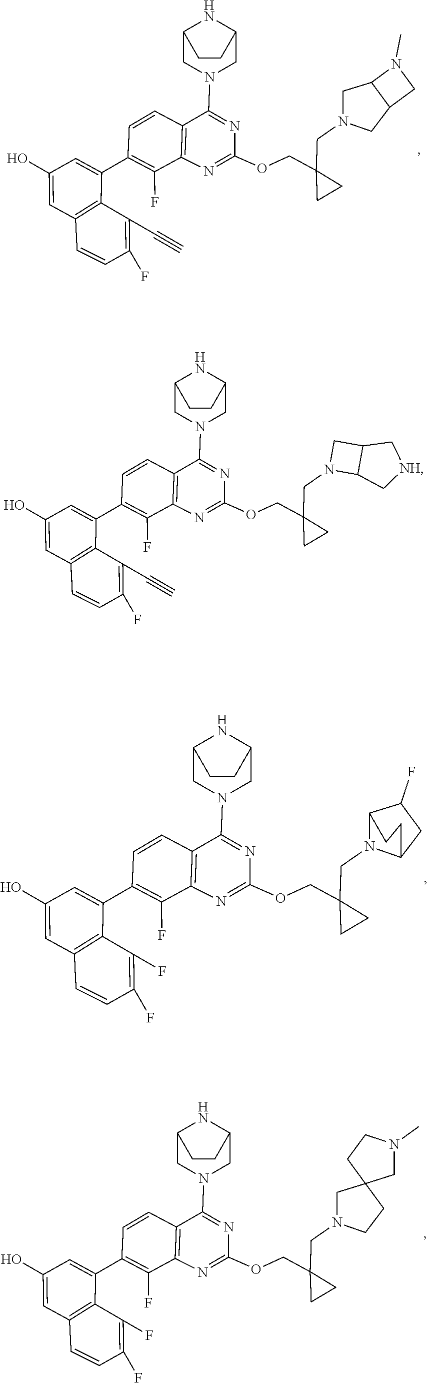 [SMILES: C#Cc1c(F)ccc2cc(O)cc(-c3ccc4c(N5CC6CCC(C5)N6)nc(OCC5(CN6CC7CN(C)C7C6)CC5)nc4c3F)c12 ; C#Cc1c(F)ccc2cc(O)cc(-c3ccc4c(N5CC6CCC(C5)N6)nc(OCC5(CN6CC7CNCC76)CC5)nc4c3F)c12 ; Oc1cc(-c2ccc3c(N4CC5CCC(C4)N5)nc(OCC4(CN5C6CCC5C(F)C6)CC4)nc3c2F)c2c(F)c(F)ccc2c1 ; CN1CCC2(CCN(CC3(COc4nc(N5CC6CCC(C5)N6)c5ccc(-c6cc(O)cc7ccc(F)c(F)c67)c(F)c5n4)CC3)C2)C1]

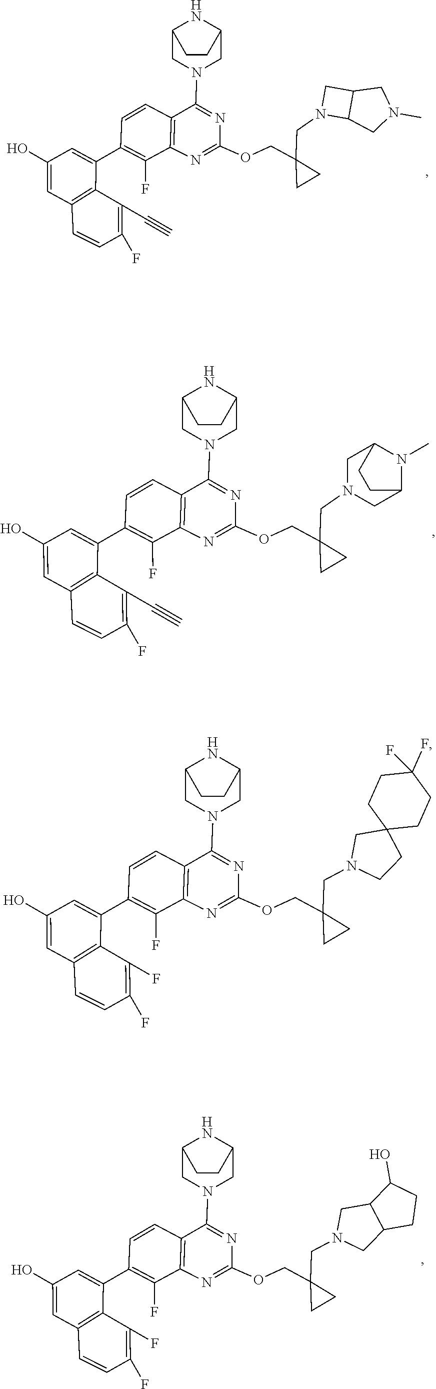 [SMILES: C#Cc1c(F)ccc2cc(O)cc(-c3ccc4c(N5CC6CCC(C5)N6)nc(OCC5(CN6CC7CN(C)CC76)CC5)nc4c3F)c12 ; C#Cc1c(F)ccc2cc(O)cc(-c3ccc4c(N5CC6CCC(C5)N6)nc(OCC5(CN6CC7CCC(C6)N7C)CC5)nc4c3F)c12 ; Oc1cc(-c2ccc3c(N4CC5CCC(C4)N5)nc(OCC4(CN5CCC6(CCC(F)(F)CC6)C5)CC4)nc3c2F)c2c(F)c(F)ccc2c1 ; Oc1cc(-c2ccc3c(N4CC5CCC(C4)N5)nc(OCC4(CN5CC6CCC(O)C6C5)CC4)nc3c2F)c2c(F)c(F)ccc2c1]

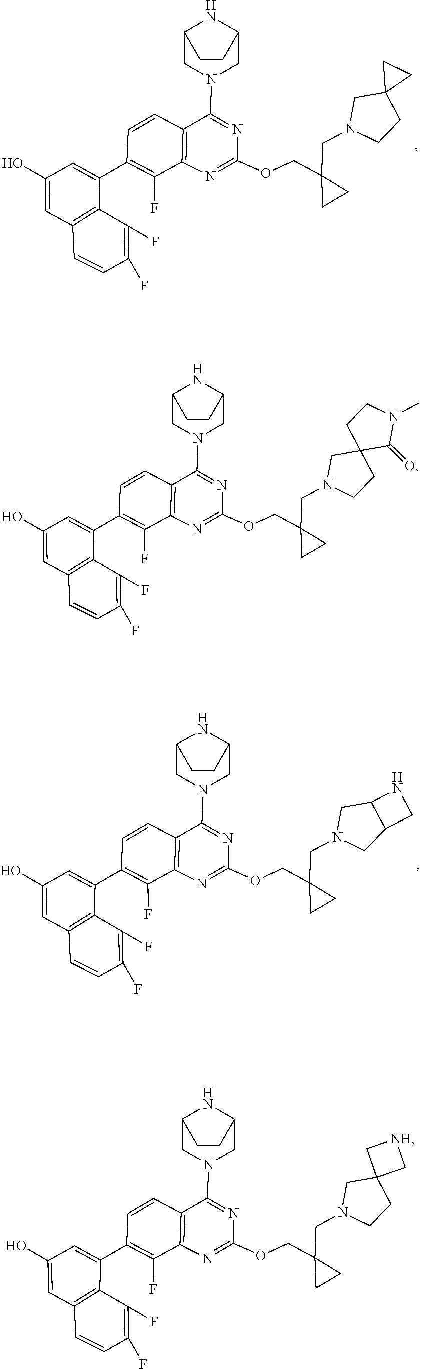 [SMILES: Oc1cc(-c2ccc3c(N4CC5CCC(C4)N5)nc(OCC4(CN5CCC6(CC6)C5)CC4)nc3c2F)c2c(F)c(F)ccc2c1 ; CN1CCC2(CCN(CC3(COc4nc(N5CC6CCC(C5)N6)c5ccc(-c6cc(O)cc7ccc(F)c(F)c67)c(F)c5n4)CC3)C2)C1=O ; Oc1cc(-c2ccc3c(N4CC5CCC(C4)N5)nc(OCC4(CN5CC6CNC6C5)CC4)nc3c2F)c2c(F)c(F)ccc2c1 ; Oc1cc(-c2ccc3c(N4CC5CCC(C4)N5)nc(OCC4(CN5CCC6(CNC6)C5)CC4)nc3c2F)c2c(F)c(F)ccc2c1]

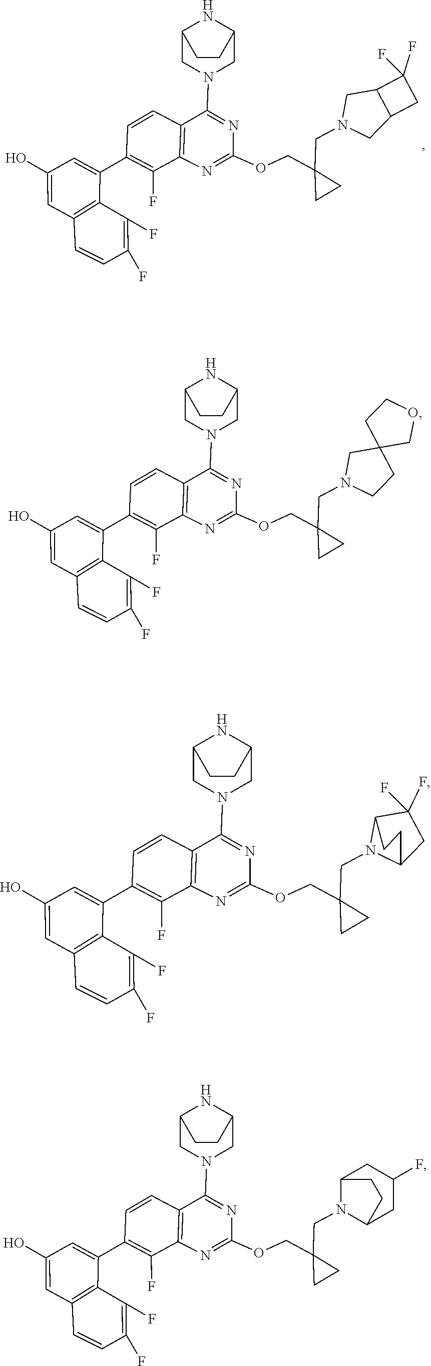 [SMILES: Oc1cc(-c2ccc3c(N4CC5CCC(C4)N5)nc(OCC4(CN5CC6CC(F)(F)C6C5)CC4)nc3c2F)c2c(F)c(F)ccc2c1 ; Oc1cc(-c2ccc3c(N4CC5CCC(C4)N5)nc(OCC4(CN5CCC6(CCOC6)C5)CC4)nc3c2F)c2c(F)c(F)ccc2c1 ; Oc1cc(-c2ccc3c(N4CC5CCC(C4)N5)nc(OCC4(CN5C6CCC5C(F)(F)C6)CC4)nc3c2F)c2c(F)c(F)ccc2c1 ; Oc1cc(-c2ccc3c(N4CC5CCC(C4)N5)nc(OCC4(CN5C6CCC5CC(F)C6)CC4)nc3c2F)c2c(F)c(F)ccc2c1]

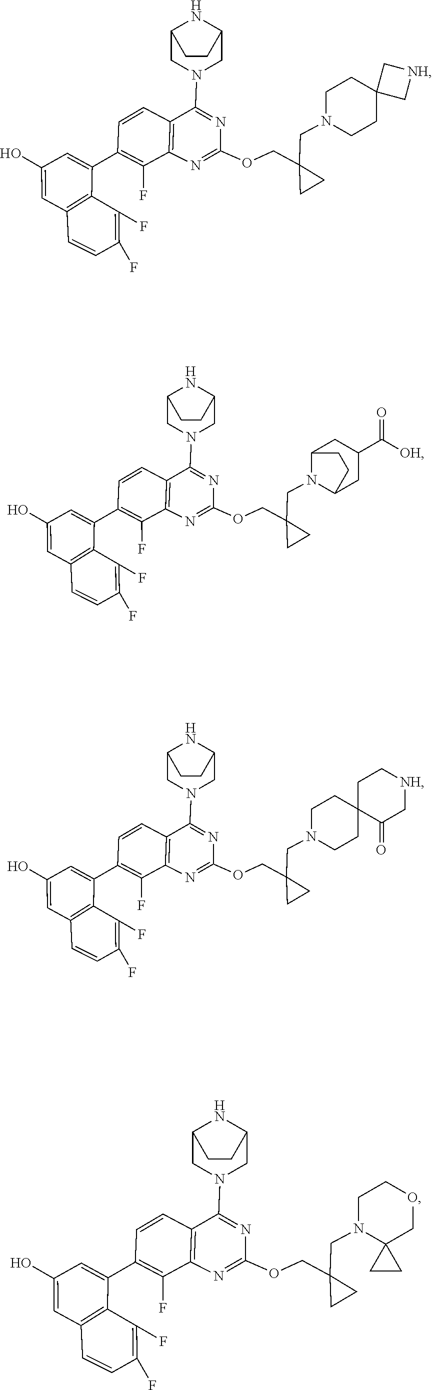 [SMILES: Oc1cc(-c2ccc3c(N4CC5CCC(C4)N5)nc(OCC4(CN5CCC6(CC5)CNC6)CC4)nc3c2F)c2c(F)c(F)ccc2c1 ; O=C(O)C1CC2CCC(C1)N2CC1(COc2nc(N3CC4CCC(C3)N4)c3ccc(-c4cc(O)cc5ccc(F)c(F)c45)c(F)c3n2)CC1 ; O=C1CNCCC12CCN(CC1(COc3nc(N4CC5CCC(C4)N5)c4ccc(-c5cc(O)cc6ccc(F)c(F)c56)c(F)c4n3)CC1)CC2 ; Oc1cc(-c2ccc3c(N4CC5CCC(C4)N5)nc(OCC4(CN5CCOCC56CC6)CC4)nc3c2F)c2c(F)c(F)ccc2c1]

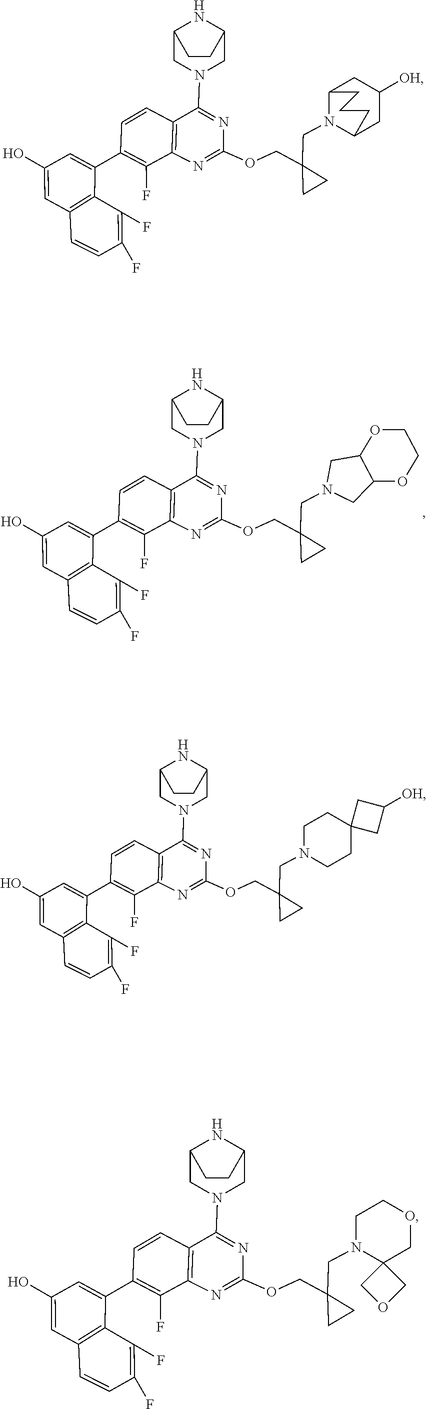 [SMILES: Oc1cc(-c2ccc3c(N4CC5CCC(C4)N5)nc(OCC4(CN5C6CCCC5CC(O)C6)CC4)nc3c2F)c2c(F)c(F)ccc2c1 ; Oc1cc(-c2ccc3c(N4CC5CCC(C4)N5)nc(OCC4(CN5CC6OCCOC6C5)CC4)nc3c2F)c2c(F)c(F)ccc2c1 ; Oc1cc(-c2ccc3c(N4CC5CCC(C4)N5)nc(OCC4(CN5CCC6(CC5)CC(O)C6)CC4)nc3c2F)c2c(F)c(F)ccc2c1 ; Oc1cc(-c2ccc3c(N4CC5CCC(C4)N5)nc(OCC4(CN5CCOCC56COC6)CC4)nc3c2F)c2c(F)c(F)ccc2c1]

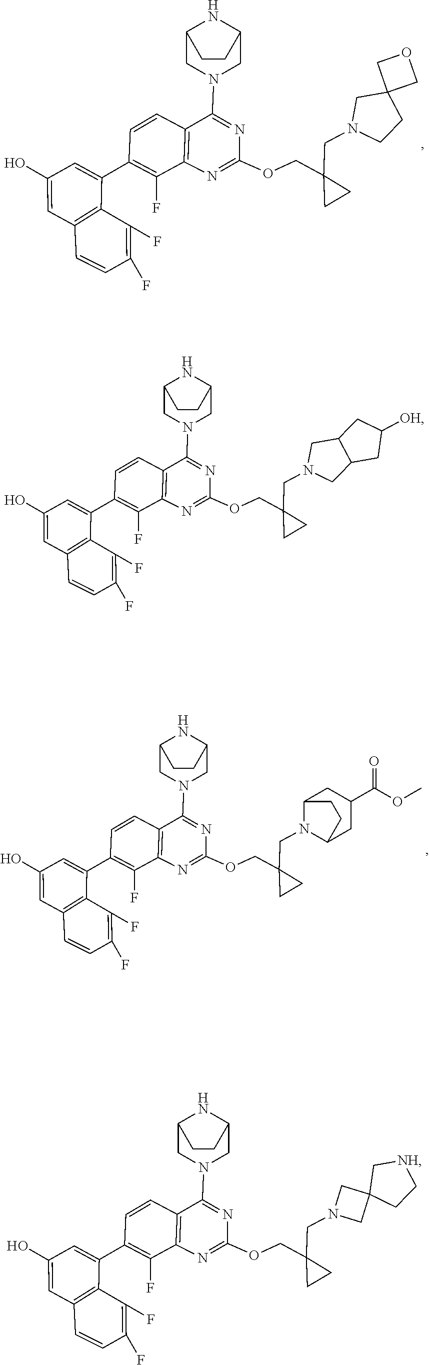 [SMILES: Oc1cc(-c2ccc3c(N4CC5CCC(C4)N5)nc(OCC4(CN5CCC6(COC6)C5)CC4)nc3c2F)c2c(F)c(F)ccc2c1 ; Oc1cc(-c2ccc3c(N4CC5CCC(C4)N5)nc(OCC4(CN5CC6CC(O)CC6C5)CC4)nc3c2F)c2c(F)c(F)ccc2c1 ; COC(=O)C1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCNC6)C5)CC4)nc3c2F)c2c(F)c(F)ccc2c1]

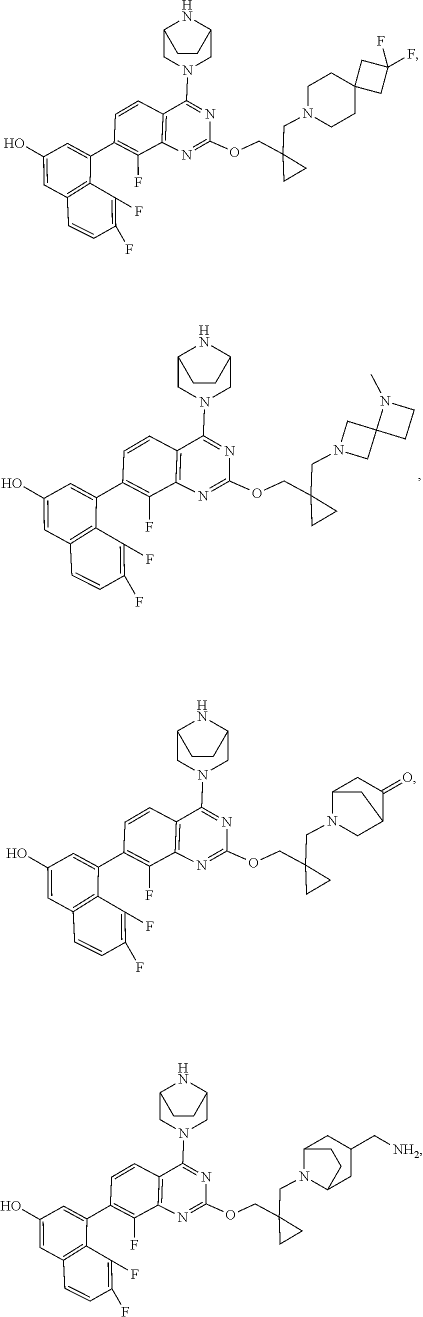 [SMILES: Oc1cc(-c2ccc3c(N4CC5CCC(C4)N5)nc(OCC4(CN5CCC6(CC5)CC(F)(F)C6)CC4)nc3c2F)c2c(F)c(F)ccc2c1 ; CN1CCC12CN(CC1(COc3nc(N4CC5CCC(C4)N5)c4ccc(-c5cc(O)cc6ccc(F)c(F)c56)c(F)c4n3)CC1)C2 ; O=C1CC2CC1CN2CC1(COc2nc(N3CC4CCC(C3)N4)c3ccc(-c4cc(O)cc5ccc(F)c(F)c45)c(F)c3n2)CC1 ; NCC1CC2CCC(C1)N2CC1(COc2nc(N3CC4CCC(C3)N4)c3ccc(-c4cc(O)cc5ccc(F)c(F)c45)c(F)c3n2)CC1]

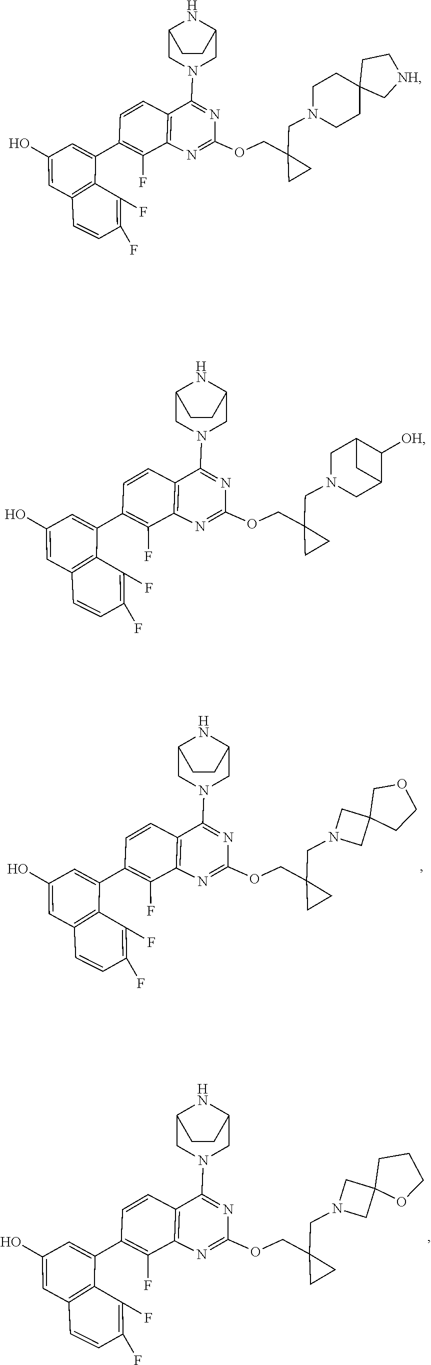 [SMILES: Oc1cc(-c2ccc3c(N4CC5CCC(C4)N5)nc(OCC4(CN5CCC6(CCNC6)CC5)CC4)nc3c2F)c2c(F)c(F)ccc2c1 ; Oc1cc(-c2ccc3c(N4CC5CCC(C4)N5)nc(OCC4(CN5CC6CC(C5)C6O)CC4)nc3c2F)c2c(F)c(F)ccc2c1 ; Oc1cc(-c2ccc3c(N4CC5CCC(C4)N5)nc(OCC4(CN5CC6(CCOC6)C5)CC4)nc3c2F)c2c(F)c(F)ccc2c1 ; Oc1cc(-c2ccc3c(N4CC5CCC(C4)N5)nc(OCC4(CN5CC6(CCCO6)C5)CC4)nc3c2F)c2c(F)c(F)ccc2c1]

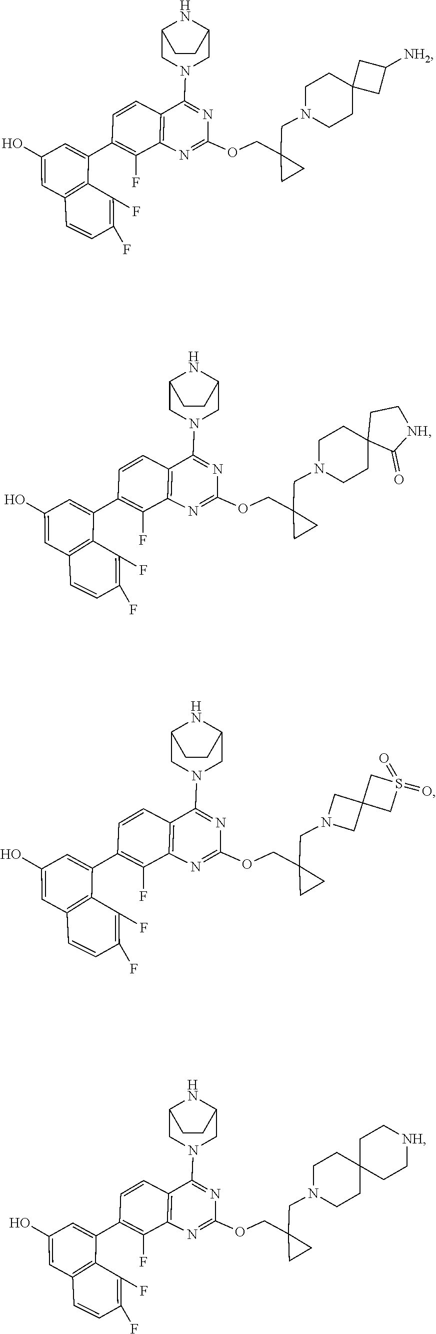 [SMILES: NC1CC2(CCN(CC3(COc4nc(N5CC6CCC(C5)N6)c5ccc(-c6cc(O)cc7ccc(F)c(F)c67)c(F)c5n4)CC3)CC2)C1 ; O=C1NCCC12CCN(CC1(COc3nc(N4CC5CCC(C4)N5)c4ccc(-c5cc(O)cc6ccc(F)c(F)c56)c(F)c4n3)CC1)CC2 ; O=S1(=O)CC2(CN(CC3(COc4nc(N5CC6CCC(C5)N6)c5ccc(-c6cc(O)cc7ccc(F)c(F)c67)c(F)c5n4)CC3)C2)C1 ; Oc1cc(-c2ccc3c(N4CC5CCC(C4)N5)nc(OCC4(CN5CCC6(CCNCC6)CC5)CC4)nc3c2F)c2c(F)c(F)ccc2c1]

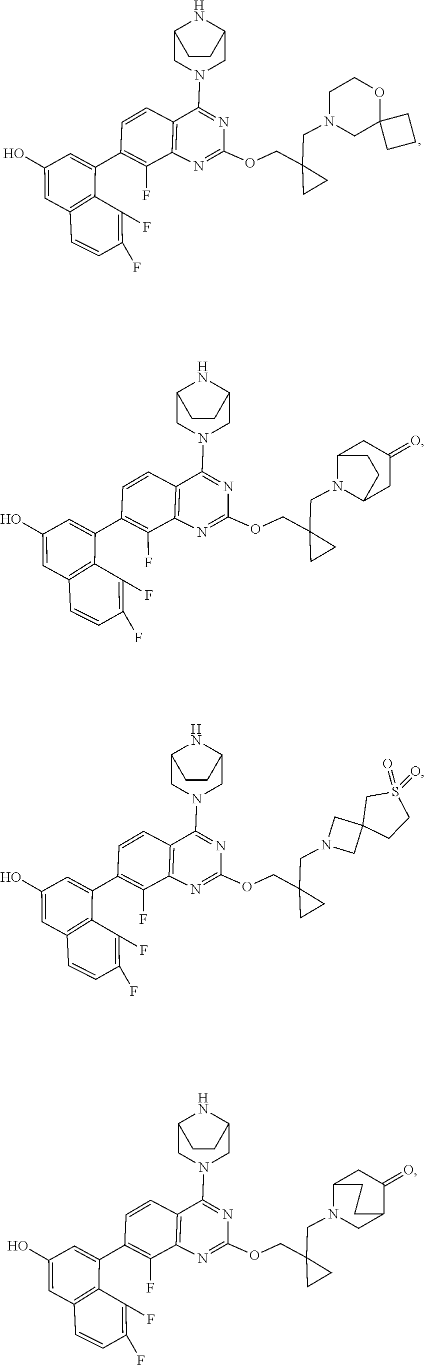 [SMILES: Oc1cc(-c2ccc3c(N4CC5CCC(C4)N5)nc(OCC4(CN5CCOC6(CCC6)C5)CC4)nc3c2F)c2c(F)c(F)ccc2c1 ; O=C1CC2CCC(C1)N2CC1(COc2nc(N3CC4CCC(C3)N4)c3ccc(-c4cc(O)cc5ccc(F)c(F)c45)c(F)c3n2)CC1 ; O=S1(=O)CCC2(CN(CC3(COc4nc(N5CC6CCC(C5)N6)c5ccc(-c6cc(O)cc7ccc(F)c(F)c67)c(F)c5n4)CC3)C2)C1 ; O=C1CC2CCC1CN2CC1(COc2nc(N3CC4CCC(C3)N4)c3ccc(-c4cc(O)cc5ccc(F)c(F)c45)c(F)c3n2)CC1]

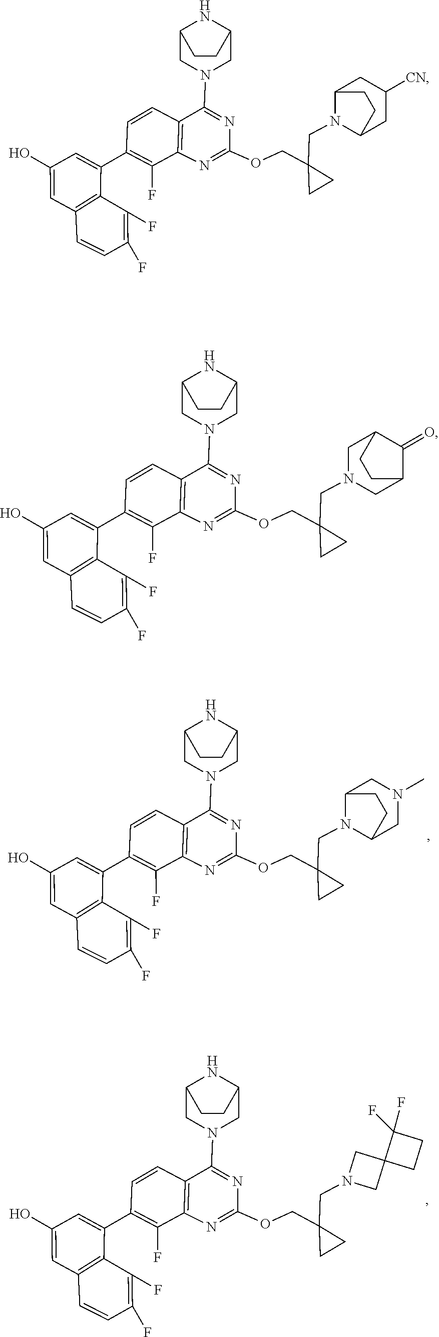 [SMILES: N#CC1CC2CCC(C1)N2CC1(COc2nc(N3CC4CCC(C3)N4)c3ccc(-c4cc(O)cc5ccc(F)c(F)c45)c(F)c3n2)CC1 ; O=C1C2CCC1CN(CC1(COc3nc(N4CC5CCC(C4)N5)c4ccc(-c5cc(O)cc6ccc(F)c(F)c56)c(F)c4n3)CC1)C2 ; CN1CC2CCC(C1)N2CC1(COc2nc(N3CC4CCC(C3)N4)c3ccc(-c4cc(O)cc5ccc(F)c(F)c45)c(F)c3n2)CC1 ; Oc1cc(-c2ccc3c(N4CC5CCC(C4)N5)nc(OCC4(CN5CC6(CCC6(F)F)C5)CC4)nc3c2F)c2c(F)c(F)ccc2c1]

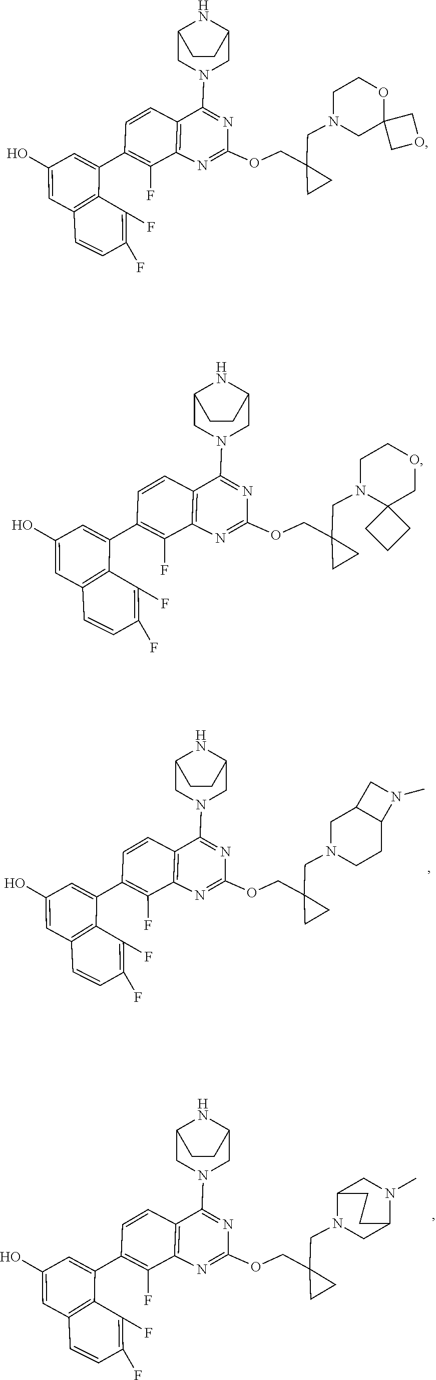 [SMILES: Oc1cc(-c2ccc3c(N4CC5CCC(C4)N5)nc(OCC4(CN5CCOC6(COC6)C5)CC4)nc3c2F)c2c(F)c(F)ccc2c1 ; Oc1cc(-c2ccc3c(N4CC5CCC(C4)N5)nc(OCC4(CN5CCOCC56CCC6)CC4)nc3c2F)c2c(F)c(F)ccc2c1 ; CN1CC2CN(CC3(COc4nc(N5CC6CCC(C5)N6)c5ccc(-c6cc(O)cc7ccc(F)c(F)c67)c(F)c5n4)CC3)CCC21 ; CN1CC2CCC1CN2CC1(COc2nc(N3CC4CCC(C3)N4)c3ccc(-c4cc(O)cc5ccc(F)c(F)c45)c(F)c3n2)CC1]

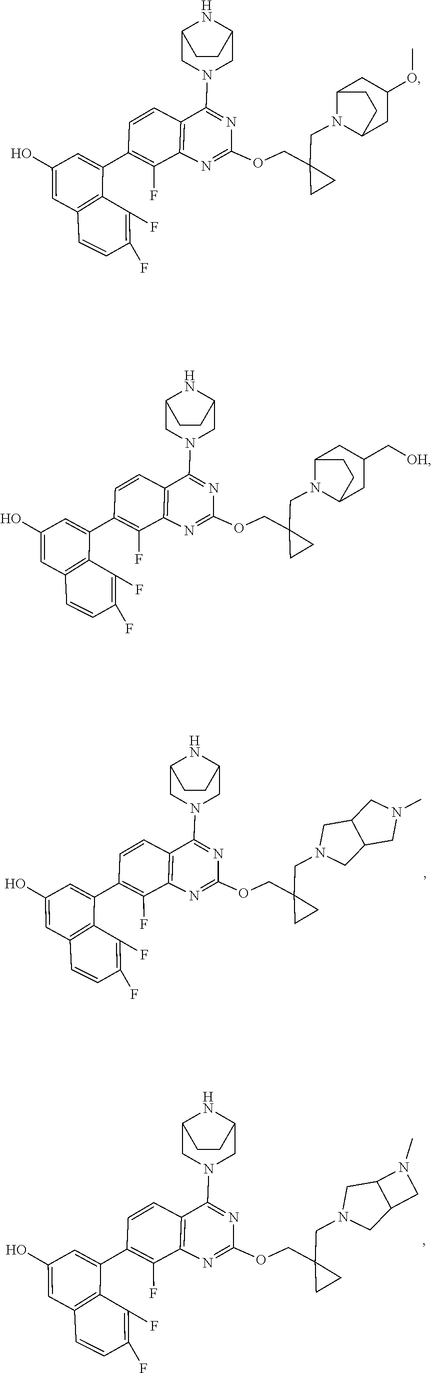 [SMILES: COC1CC2CCC(C1)N2CC1(COc2nc(N3CC4CCC(C3)N4)c3ccc(-c4cc(O)cc5ccc(F)c(F)c45)c(F)c3n2)CC1 ; OCC1CC2CCC(C1)N2CC1(COc2nc(N3CC4CCC(C3)N4)c3ccc(-c4cc(O)cc5ccc(F)c(F)c45)c(F)c3n2)CC1 ; CN1CC2CN(CC3(COc4nc(N5CC6CCC(C5)N6)c5ccc(-c6cc(O)cc7ccc(F)c(F)c67)c(F)c5n4)CC3)CC2C1 ; CN1CC2CN(CC3(COc4nc(N5CC6CCC(C5)N6)c5ccc(-c6cc(O)cc7ccc(F)c(F)c67)c(F)c5n4)CC3)CC21]

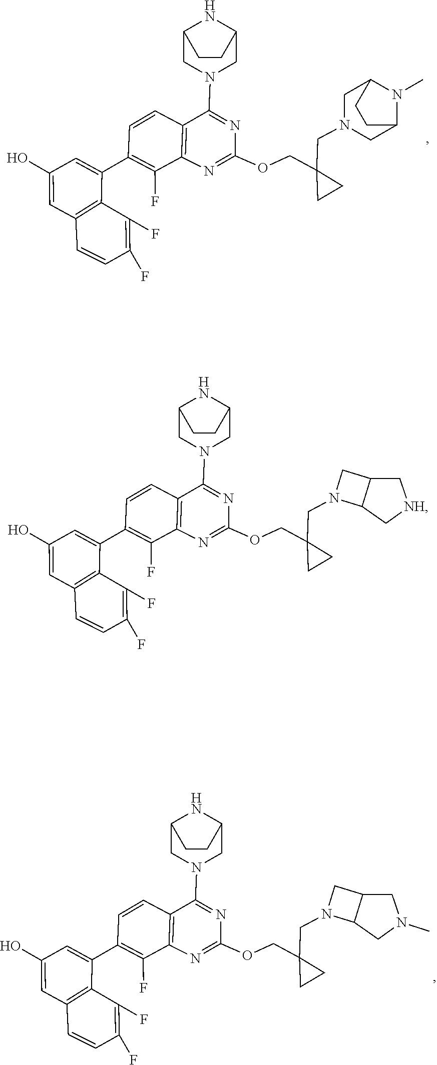 [SMILES: CN1C2CCC1CN(CC1(COc3nc(N4CC5CCC(C4)N5)c4ccc(-c5cc(O)cc6ccc(F)c(F)c56)c(F)c4n3)CC1)C2 ; Oc1cc(-c2ccc3c(N4CC5CCC(C4)N5)nc(OCC4(CN5CC6CNCC65)CC4)nc3c2F)c2c(F)c(F)ccc2c1 ; CN1CC2CN(CC3(COc4nc(N5CC6CCC(C5)N6)c5ccc(-c6cc(O)cc7ccc(F)c(F)c67)c(F)c5n4)CC3)C2C1]

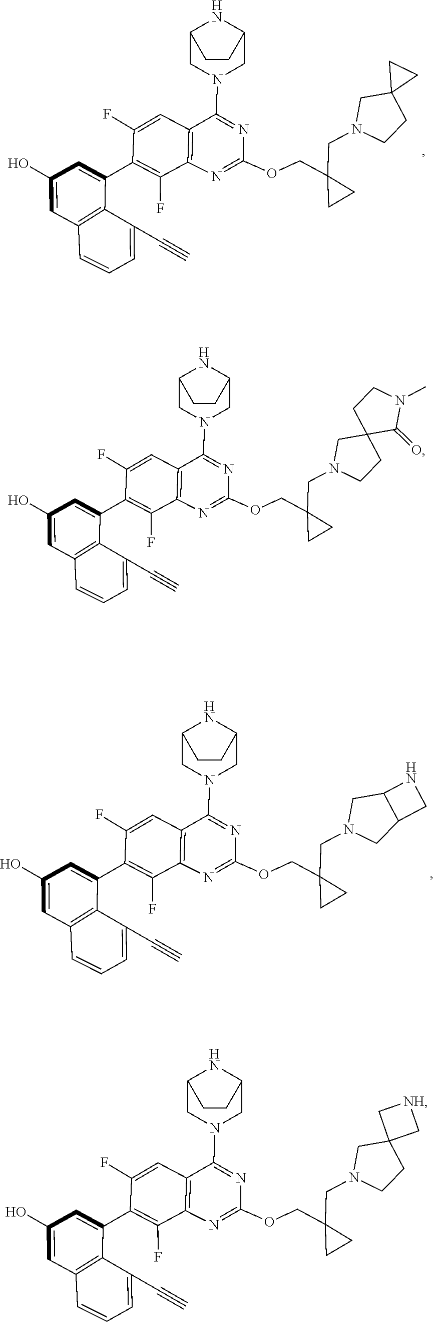

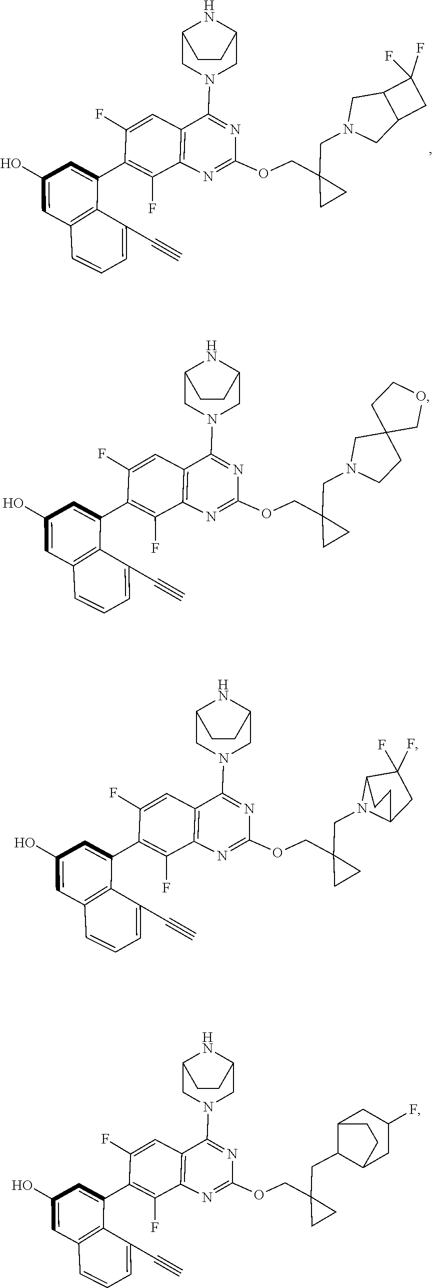

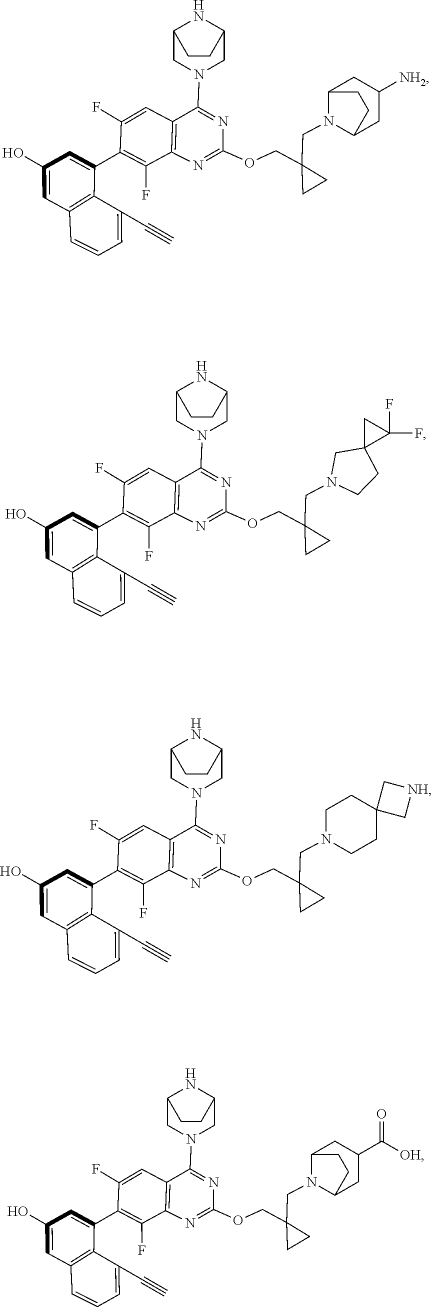

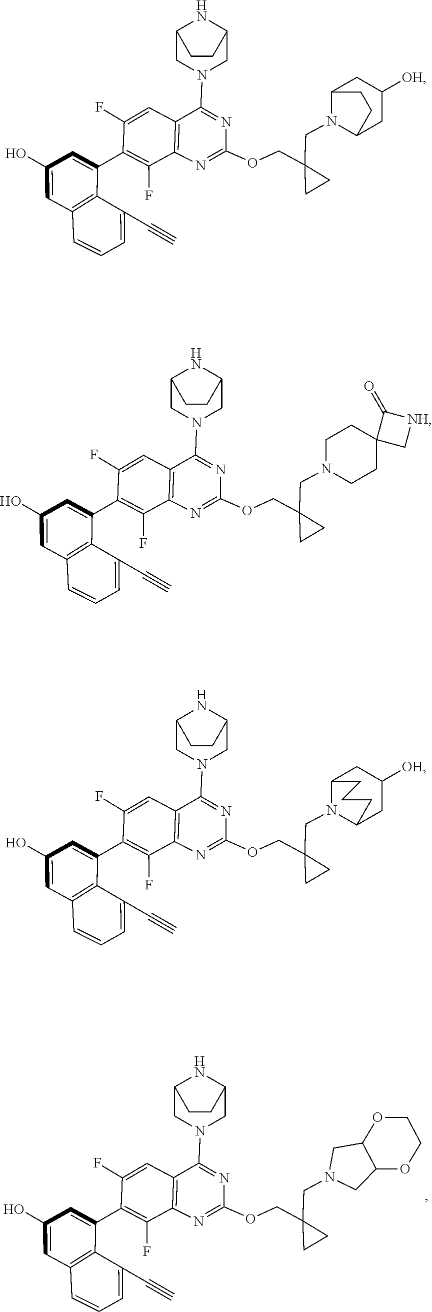

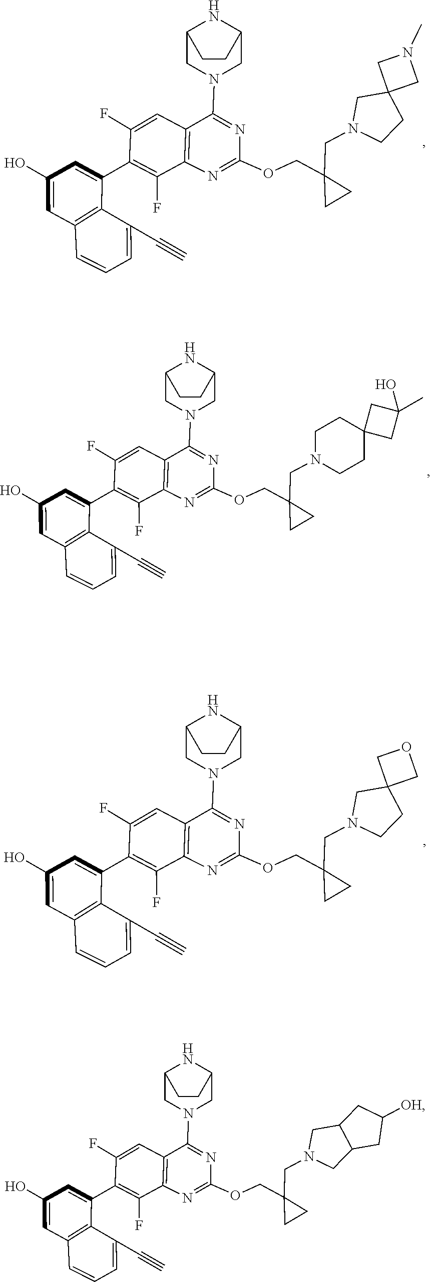

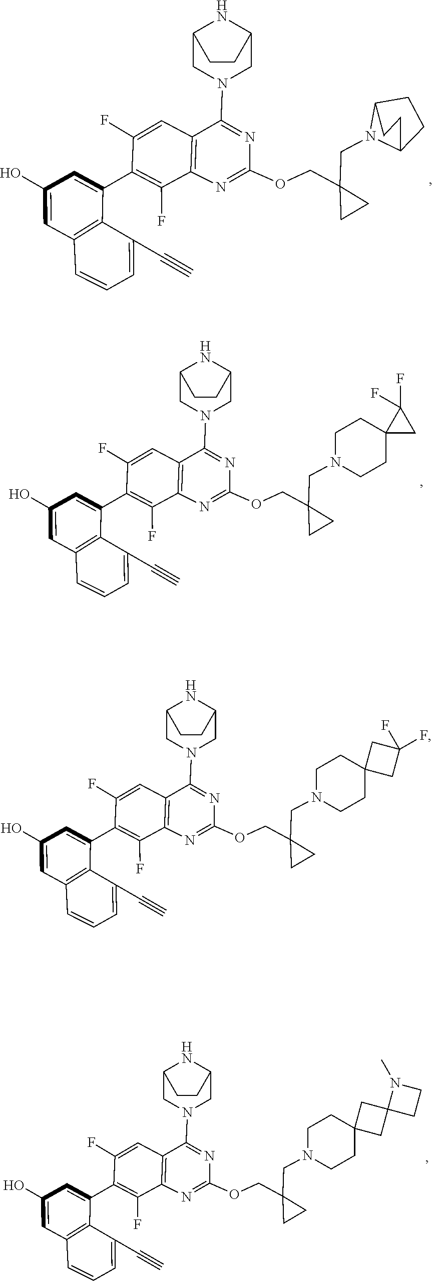

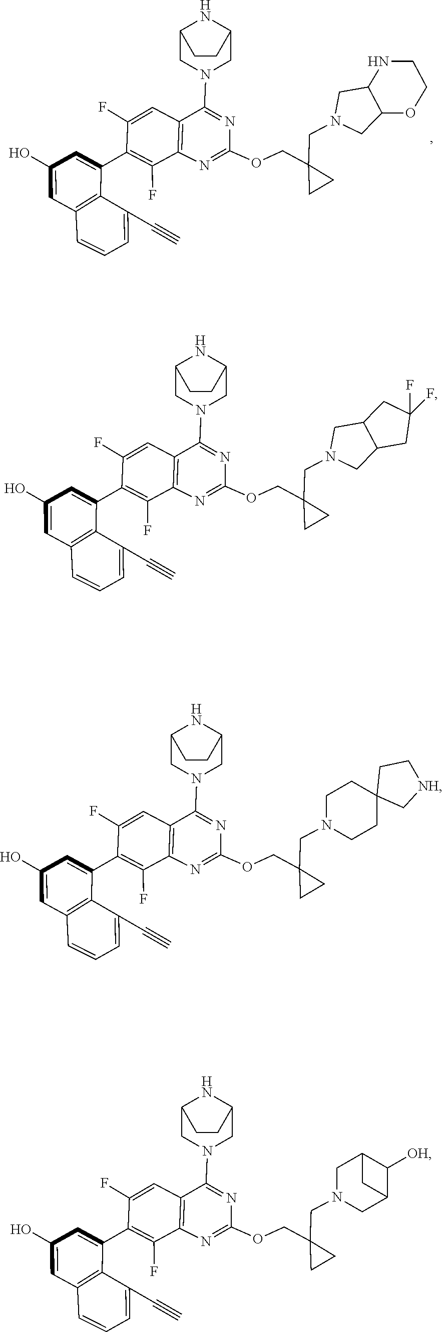

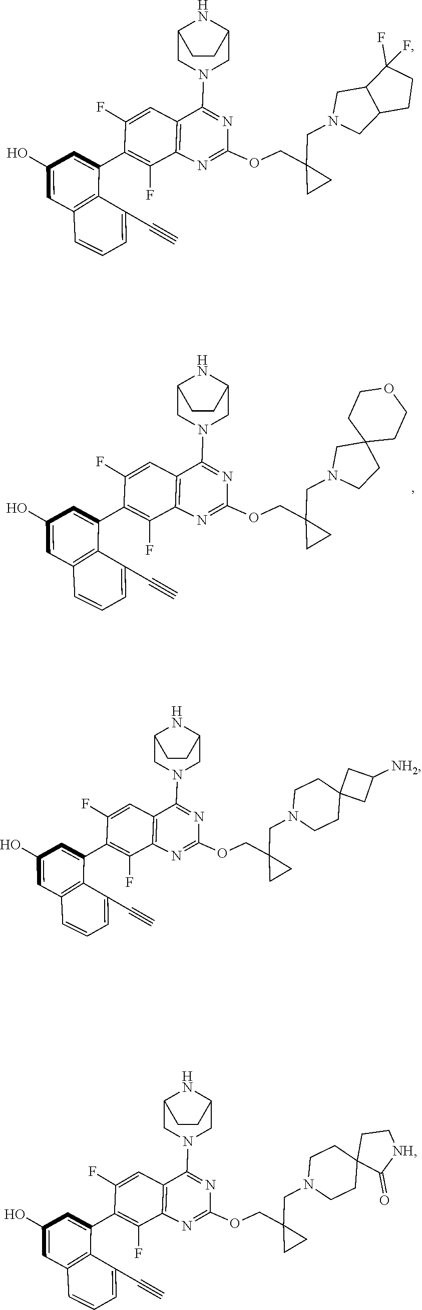

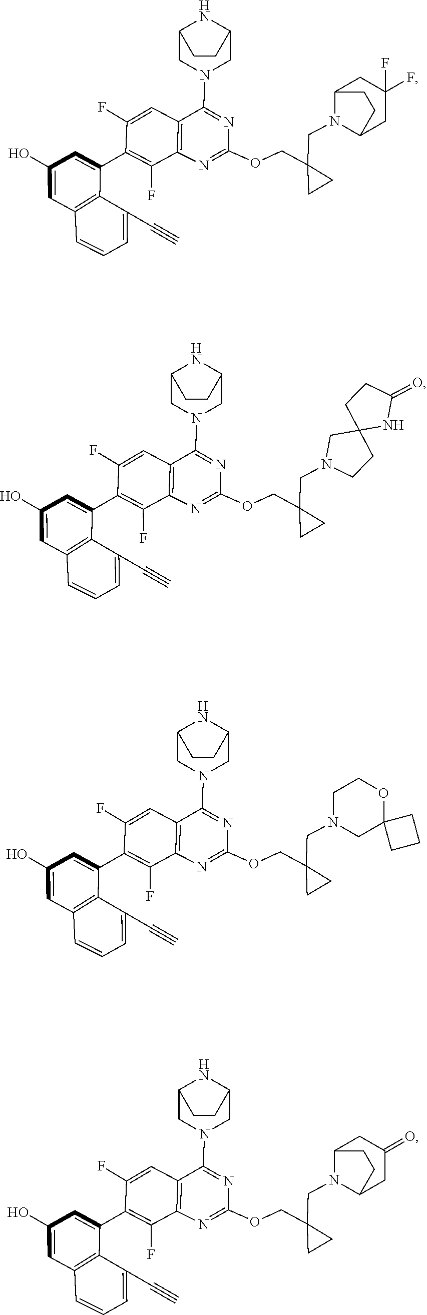

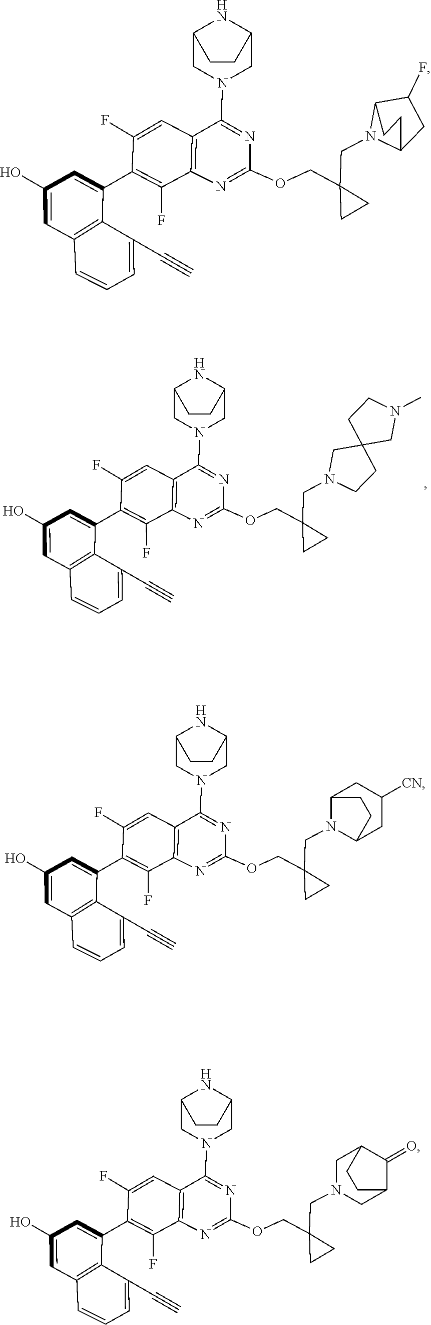

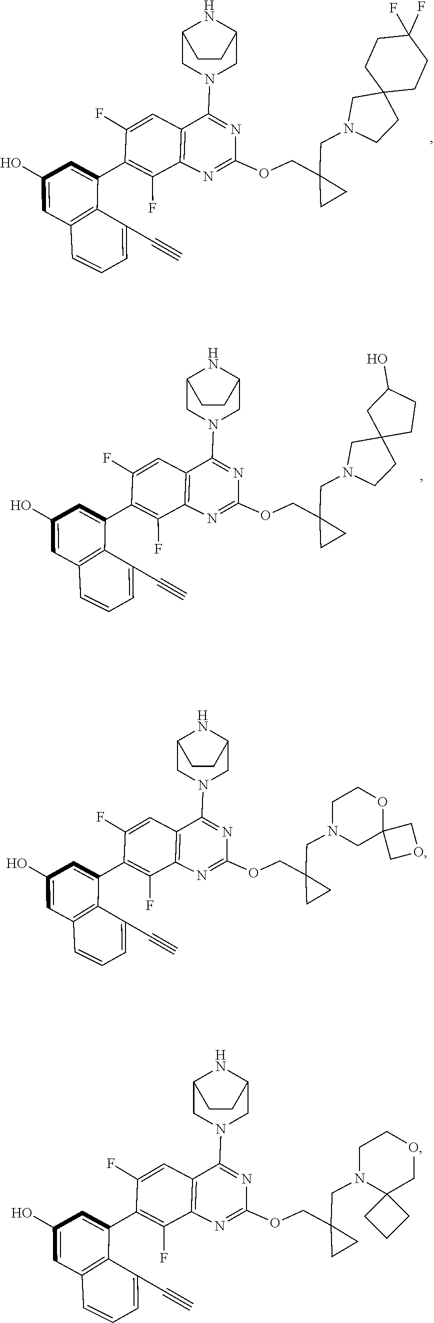

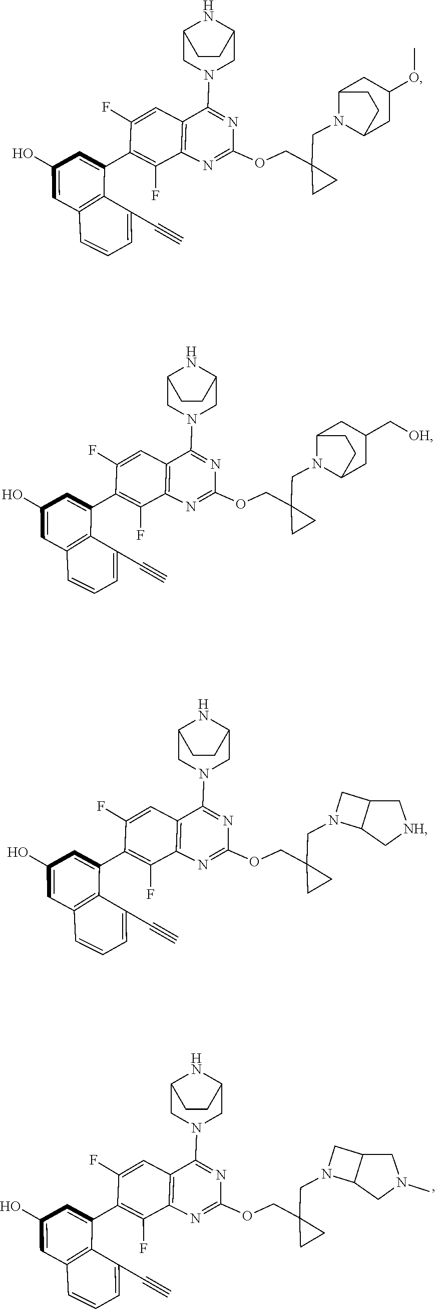

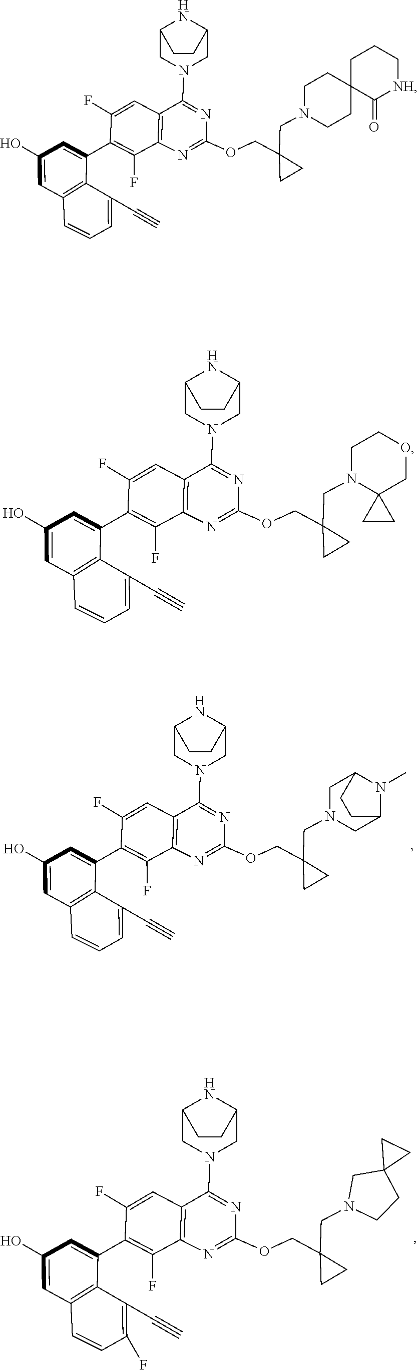

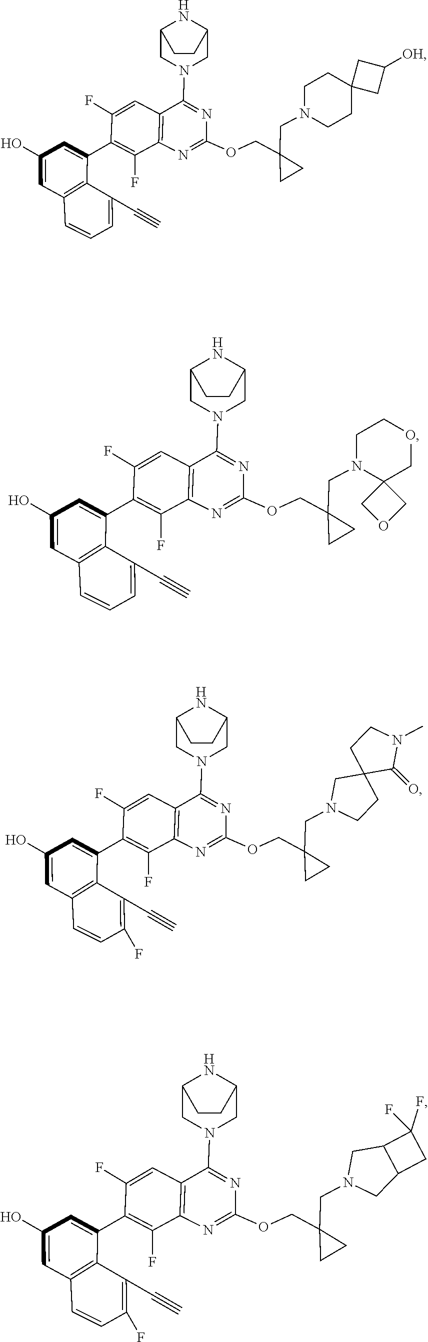

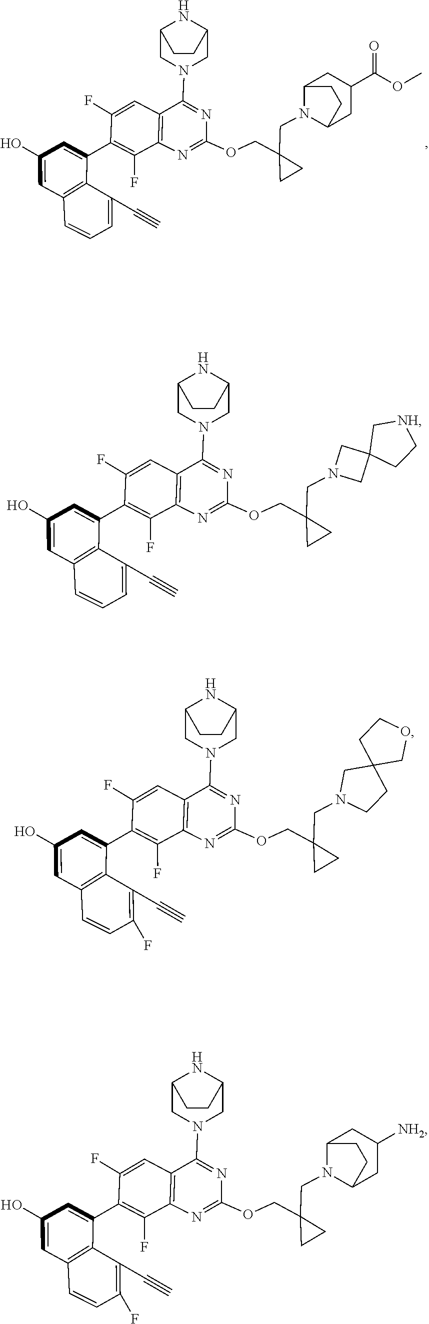

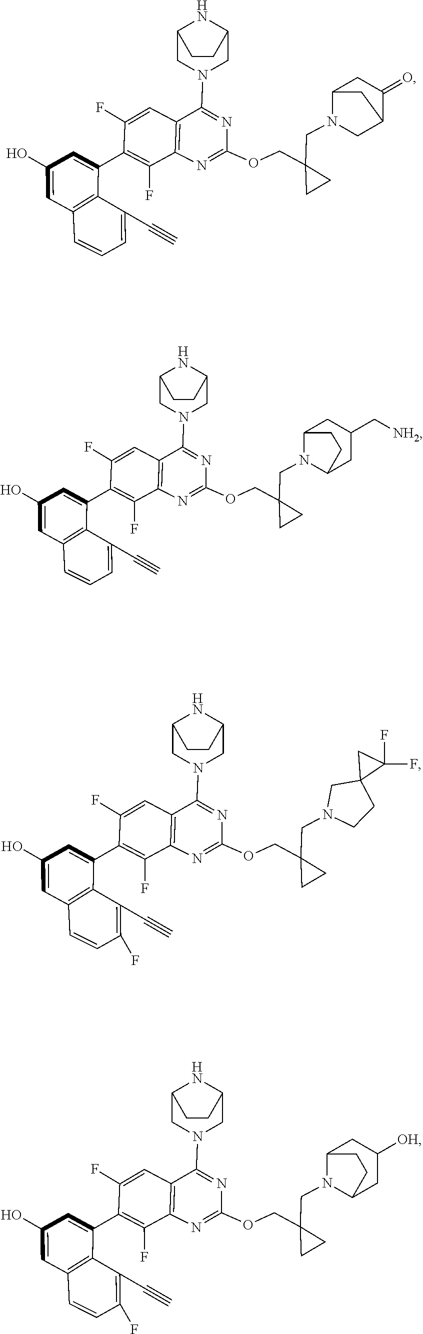

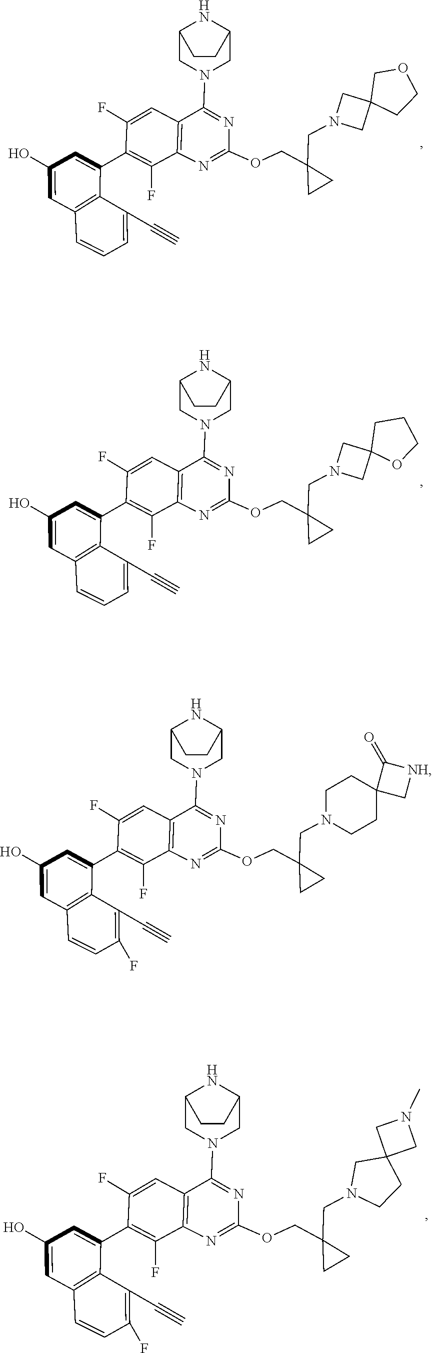

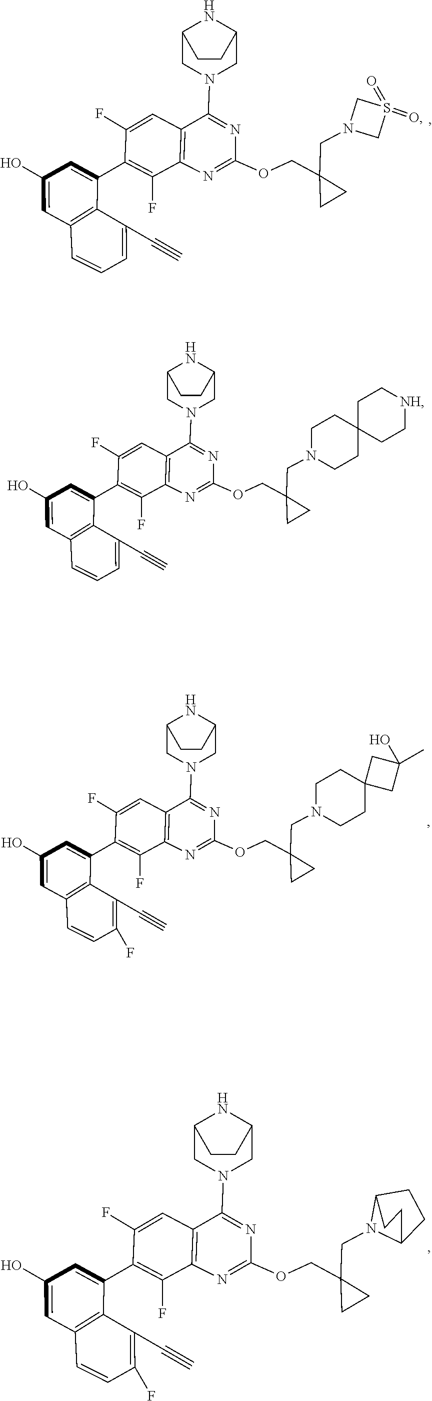

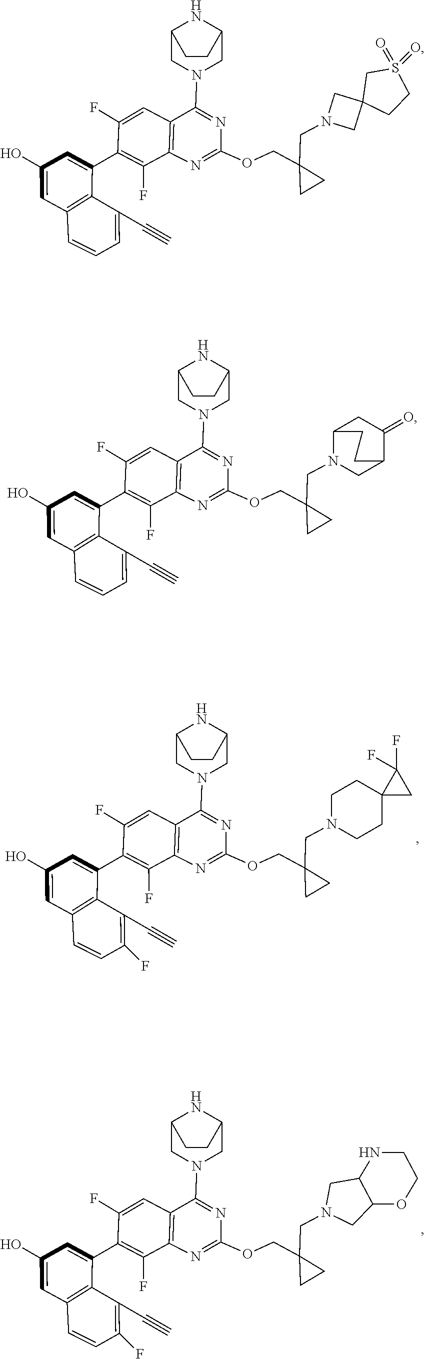

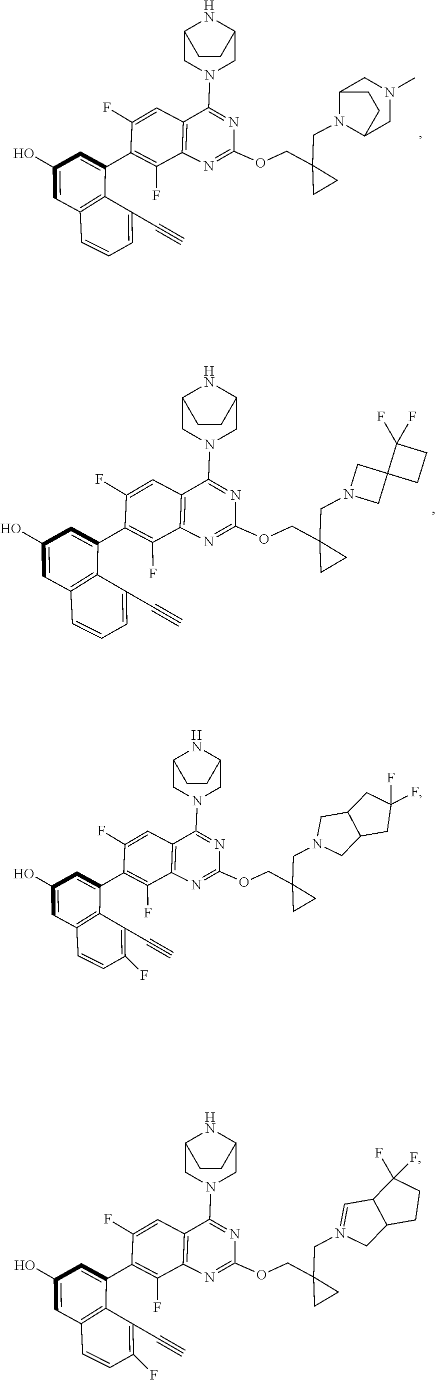

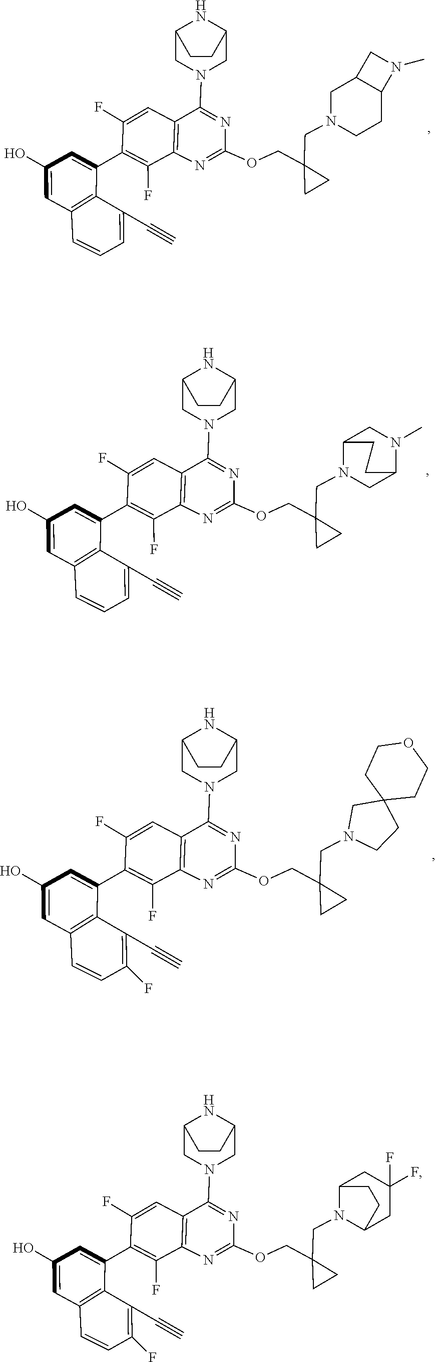

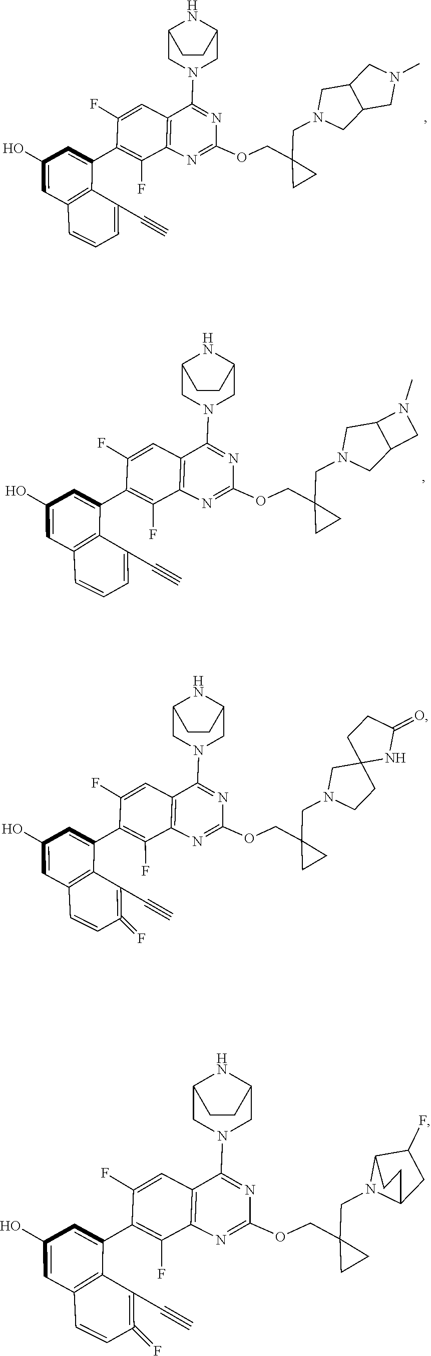

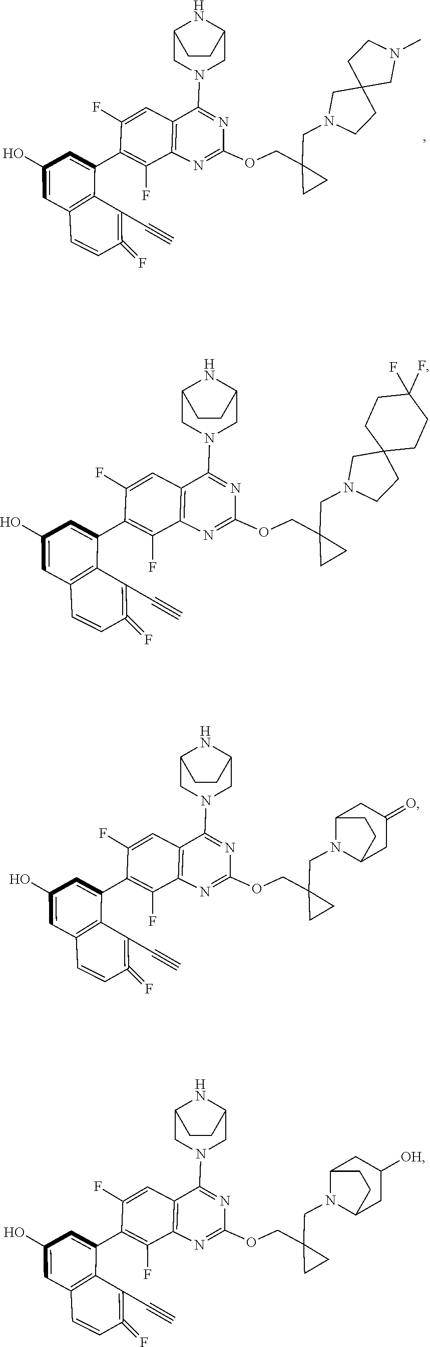

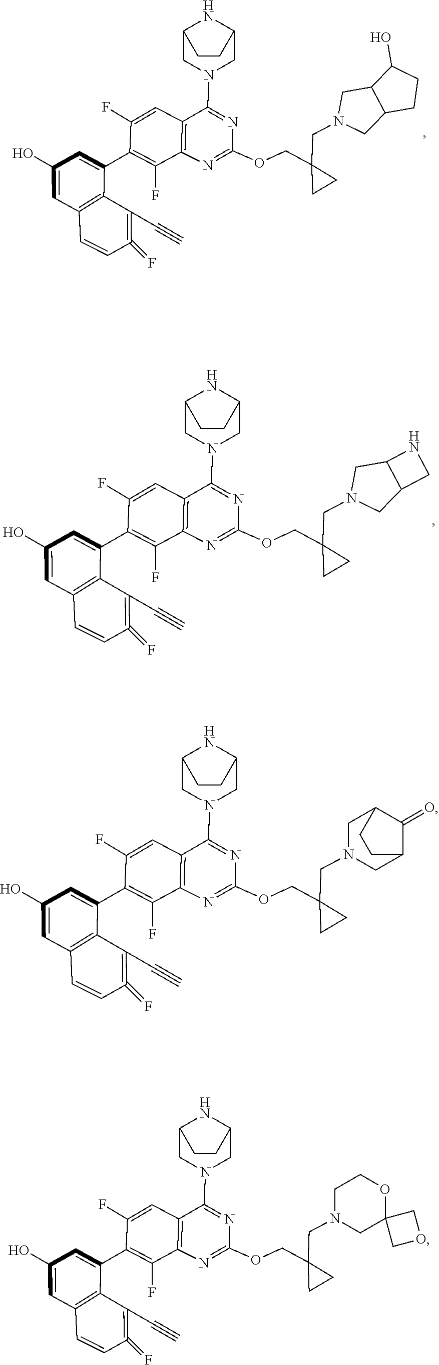

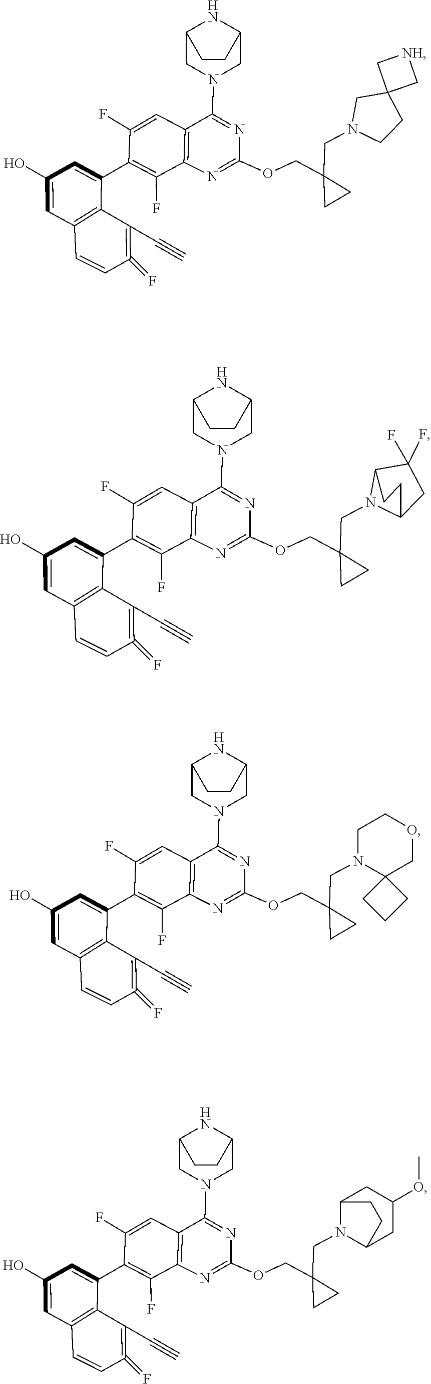

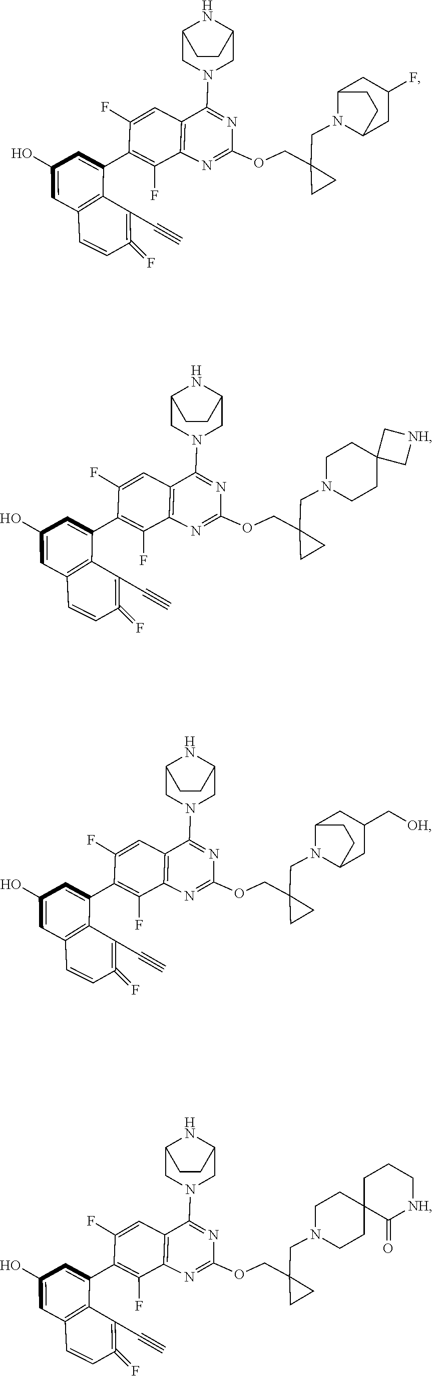

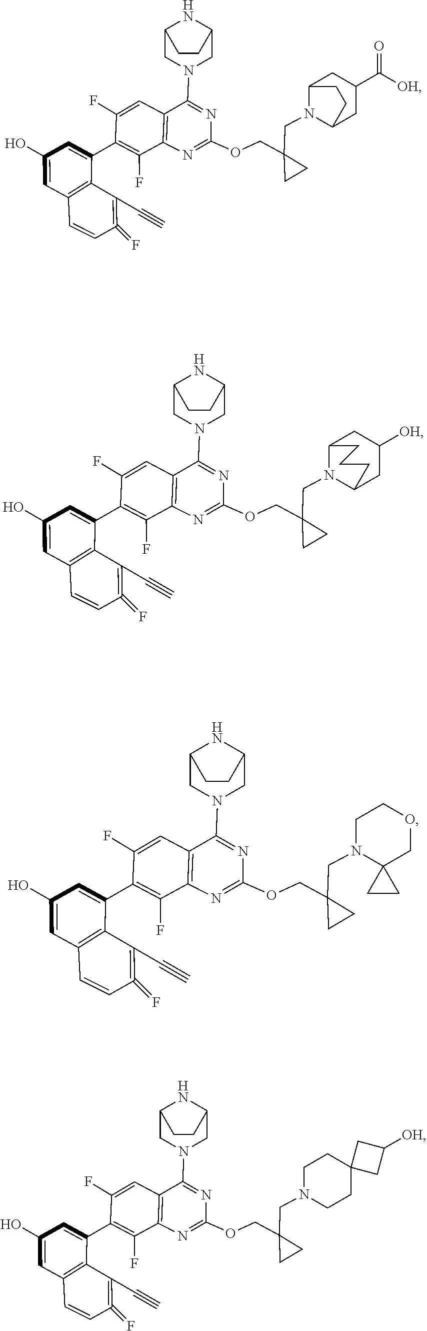

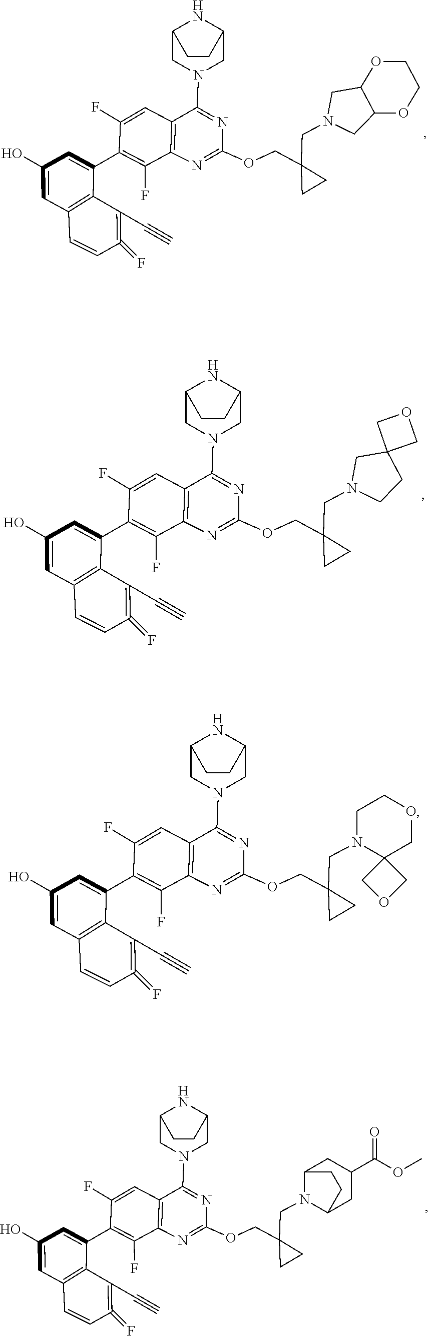

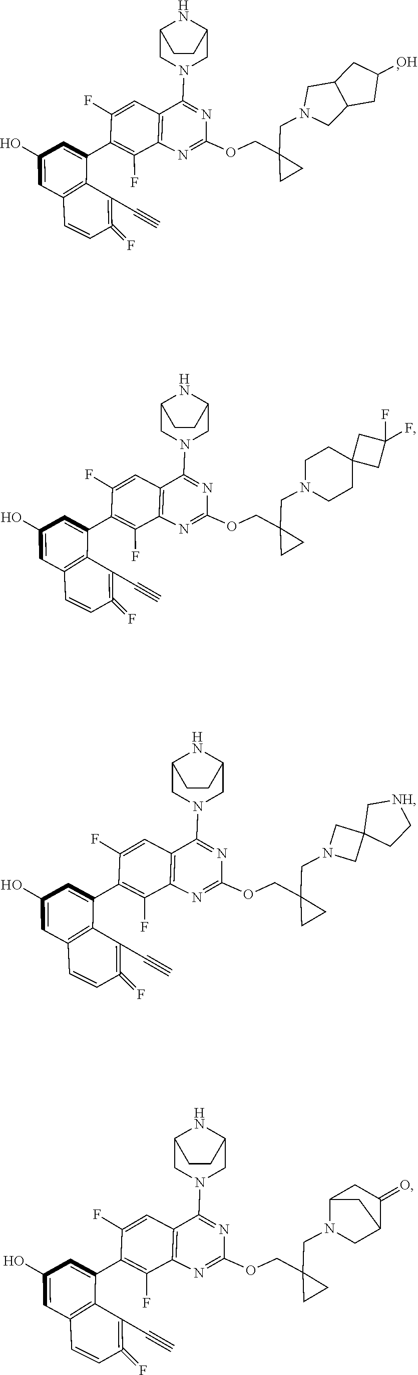

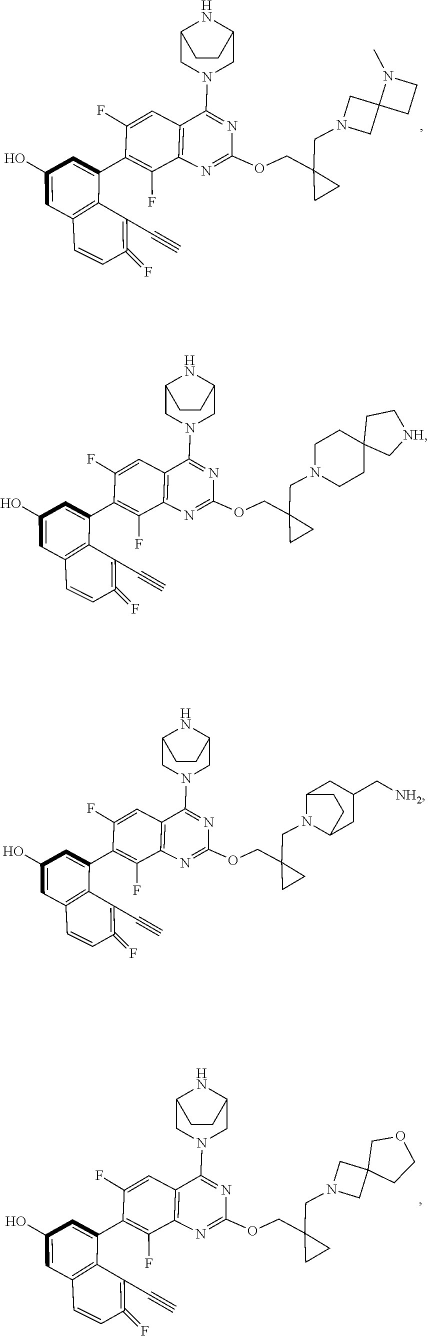

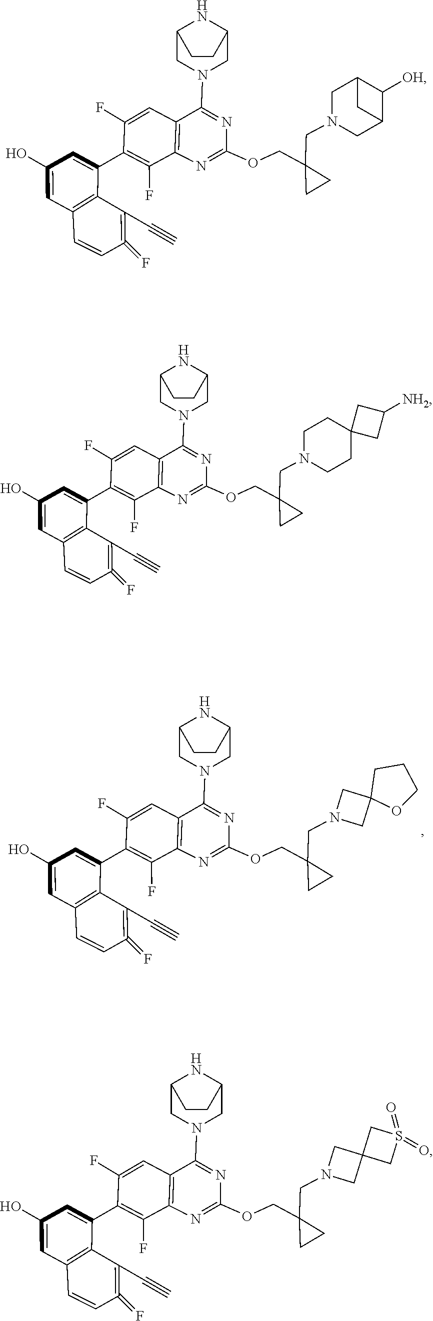

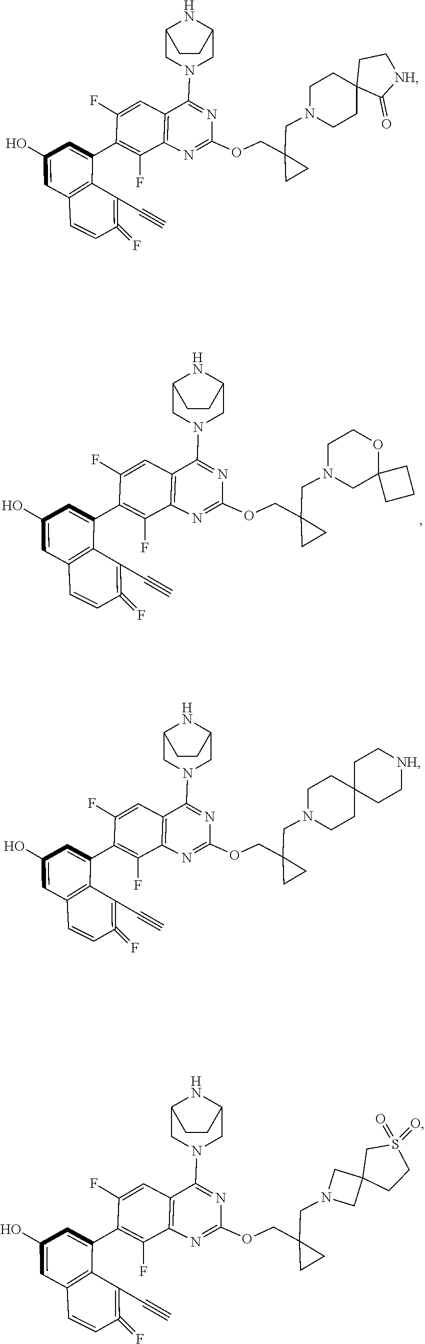

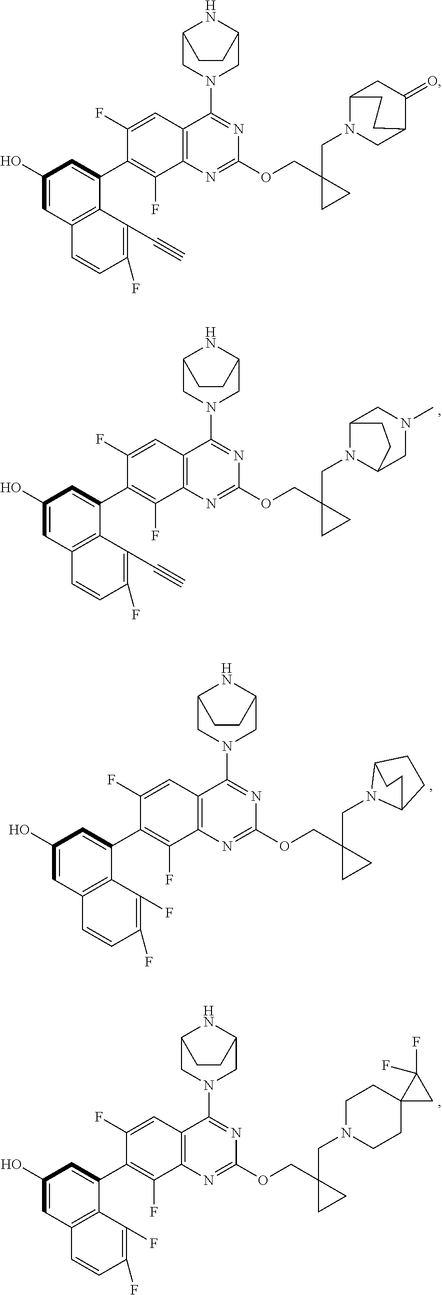

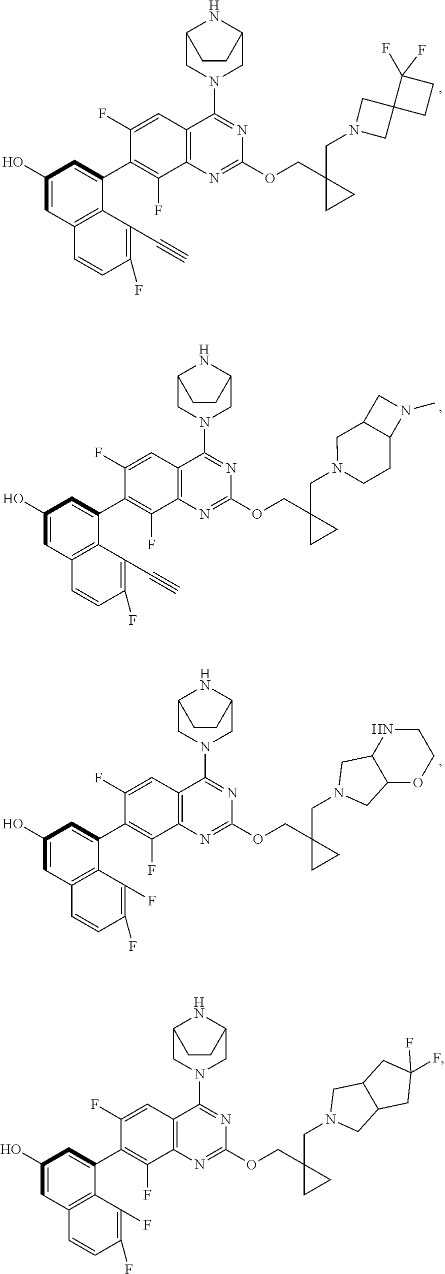

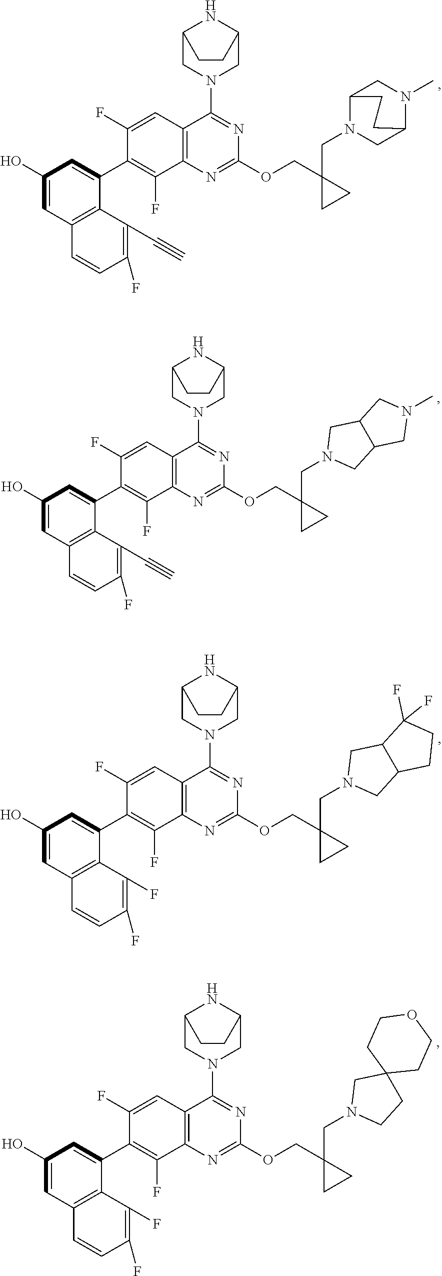

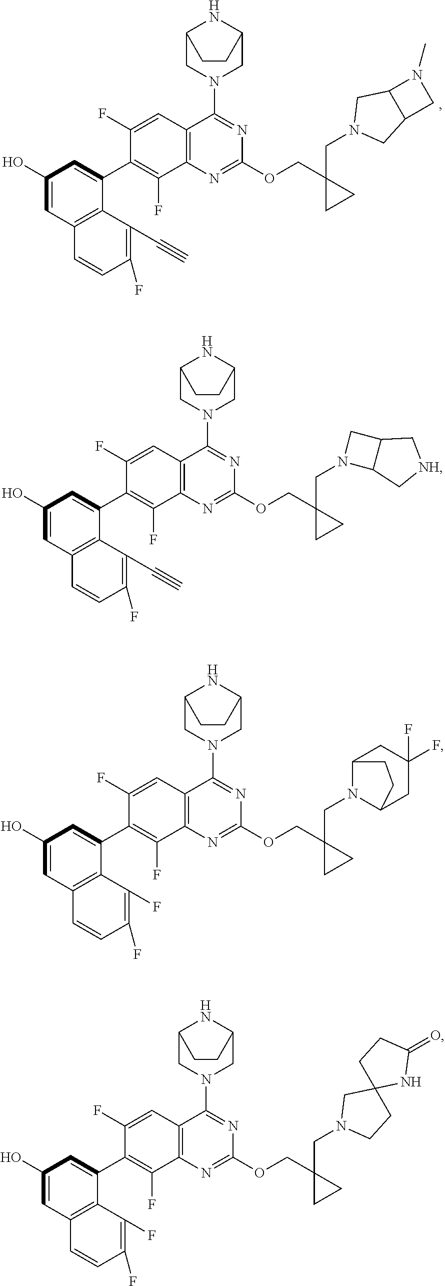

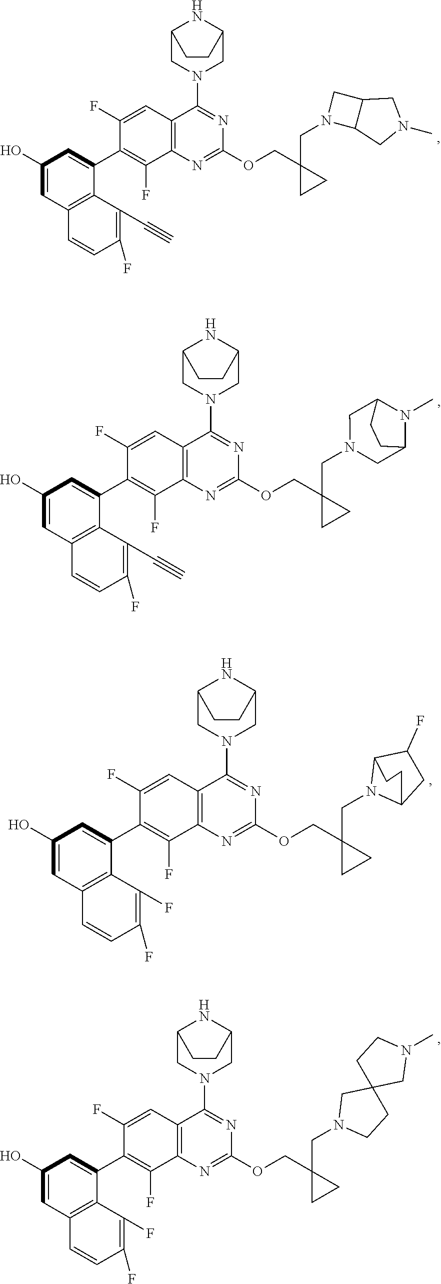

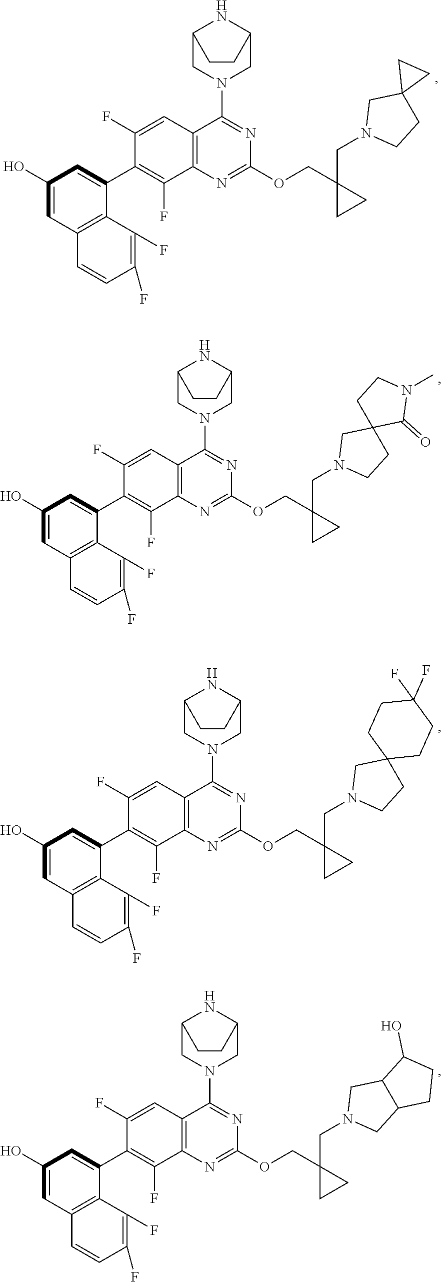

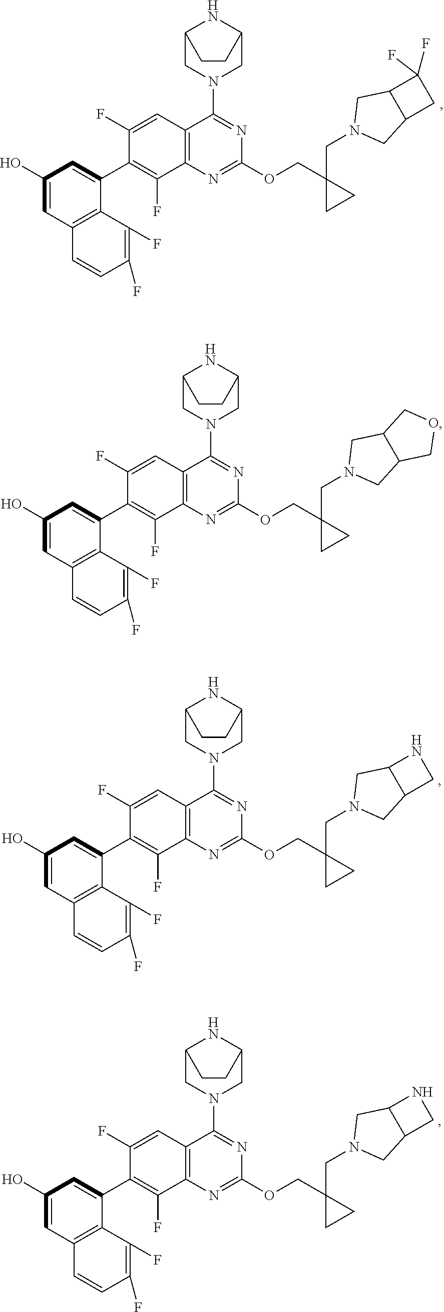

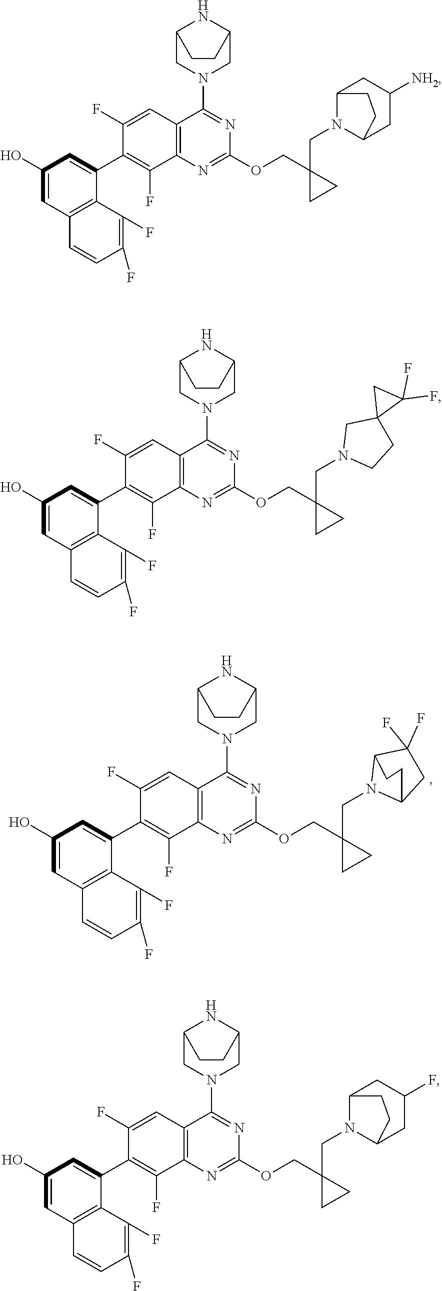

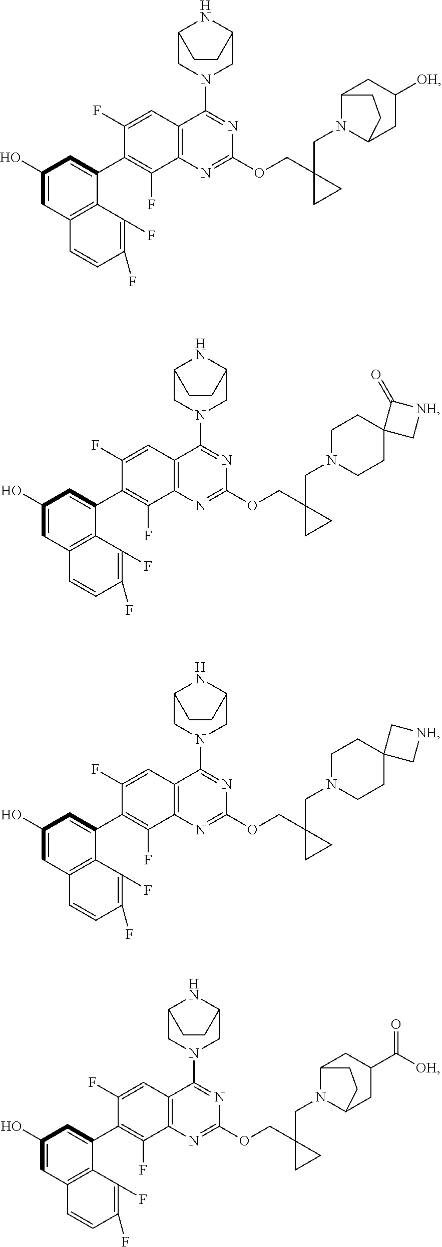

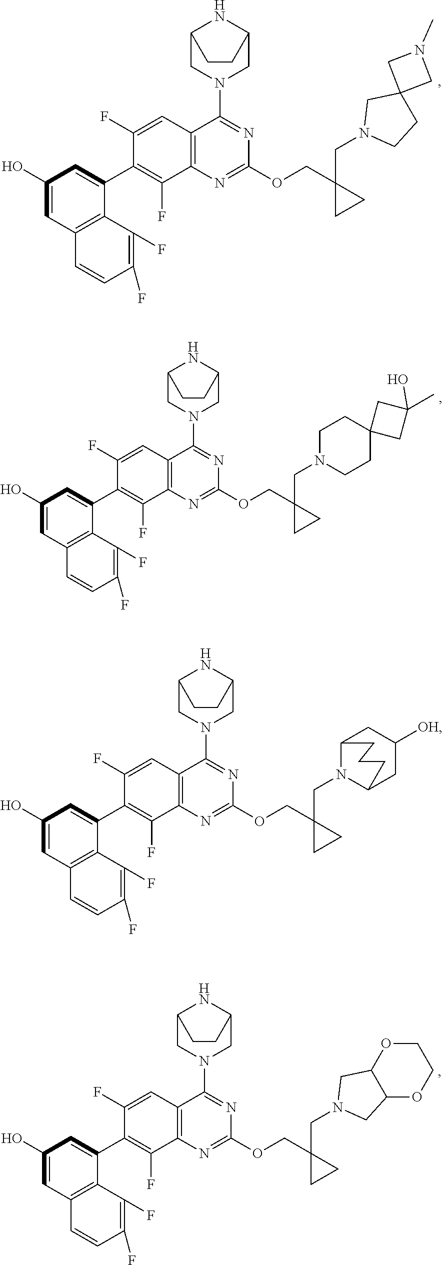

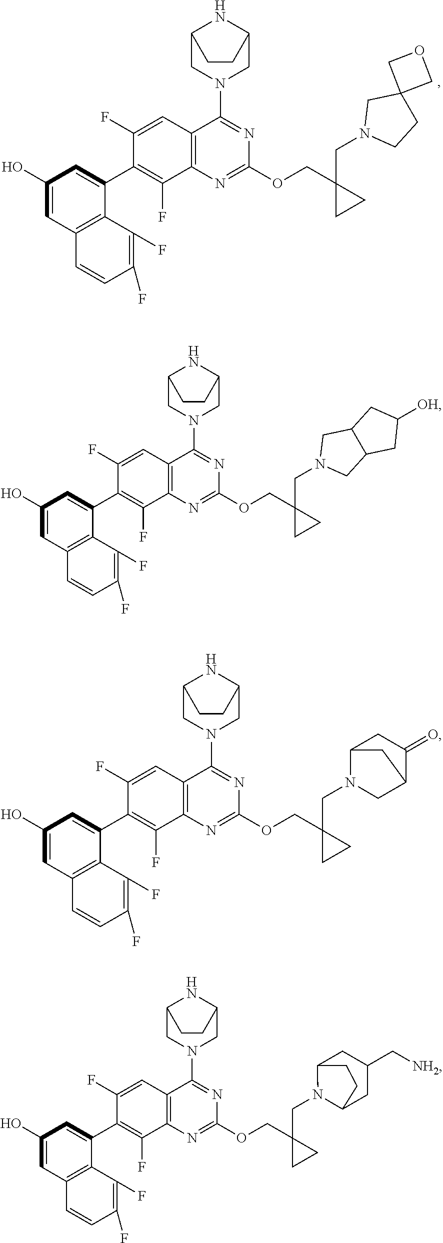

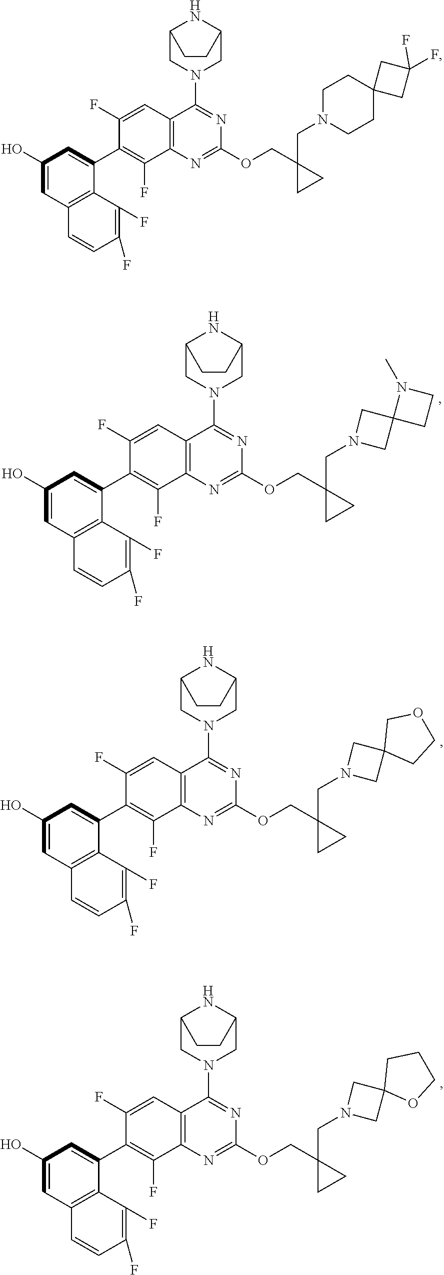

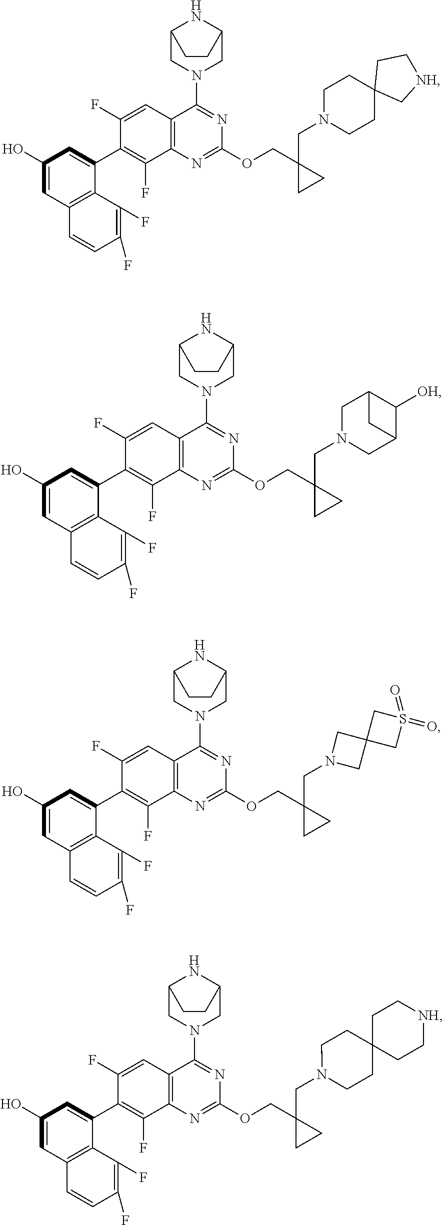

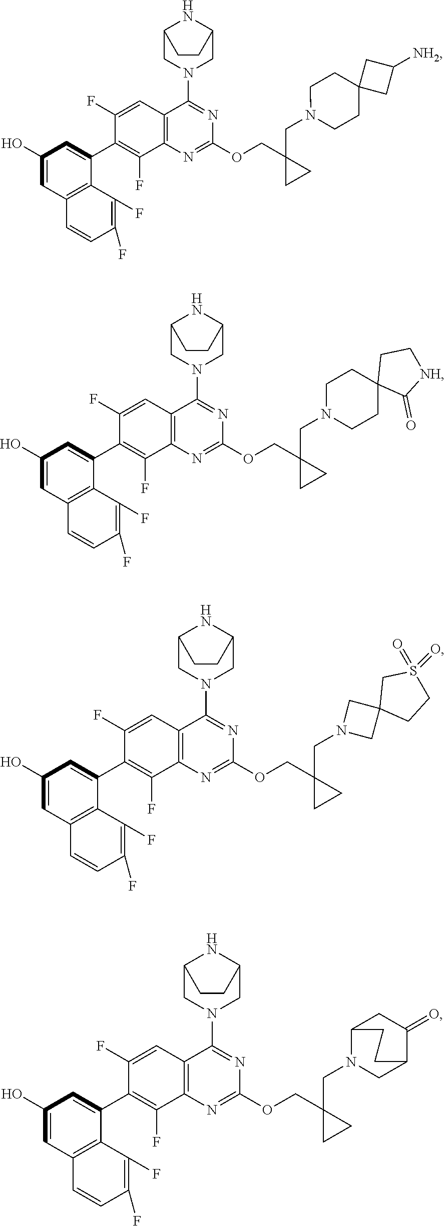

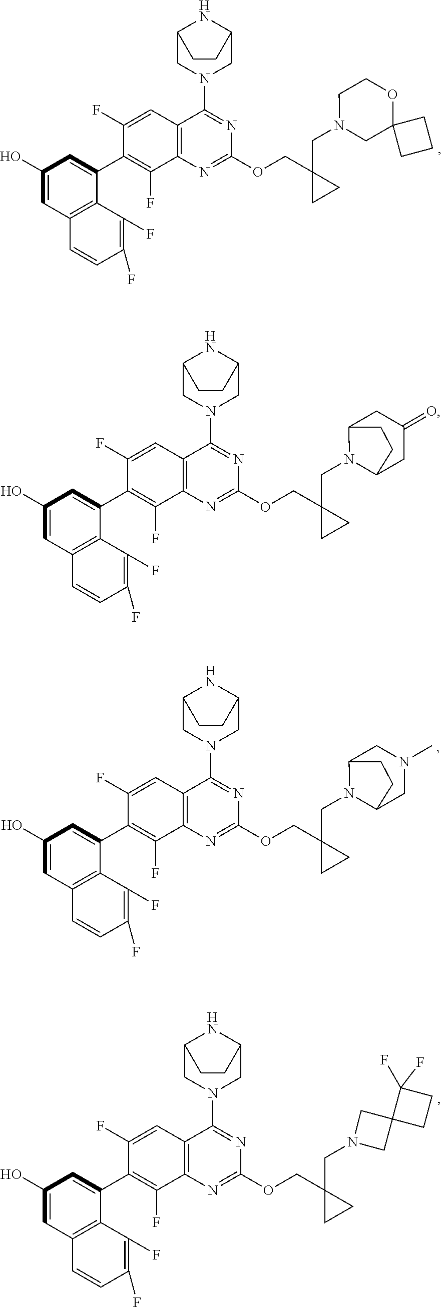

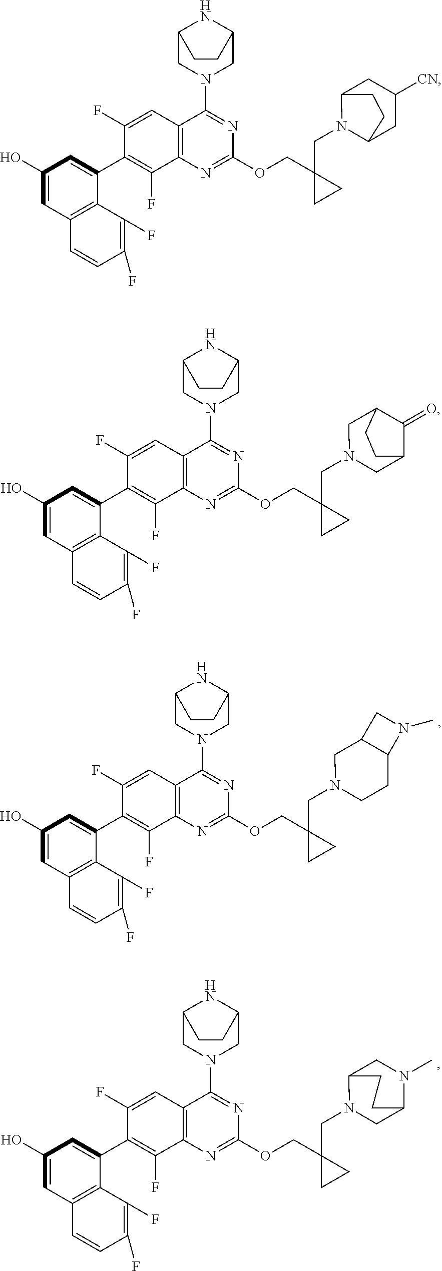

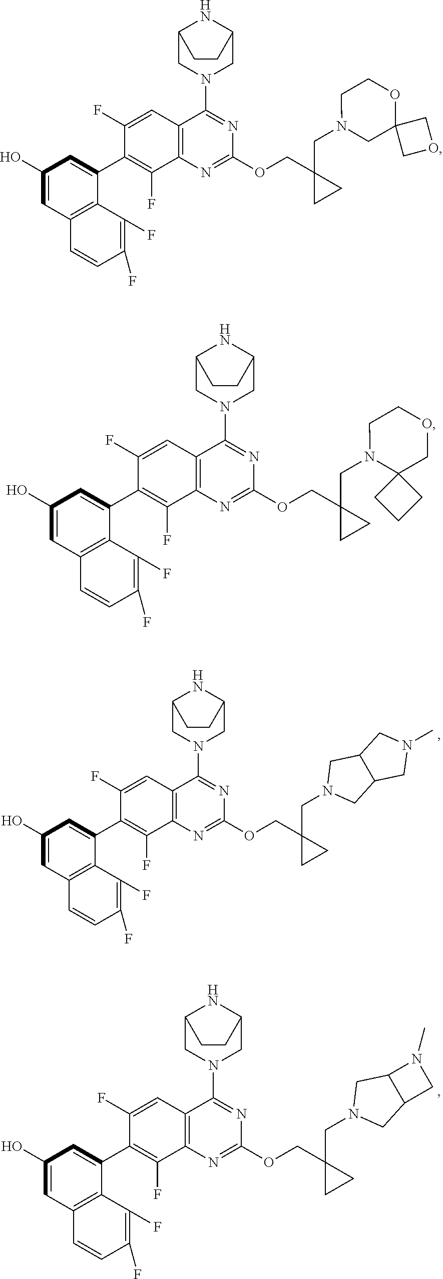

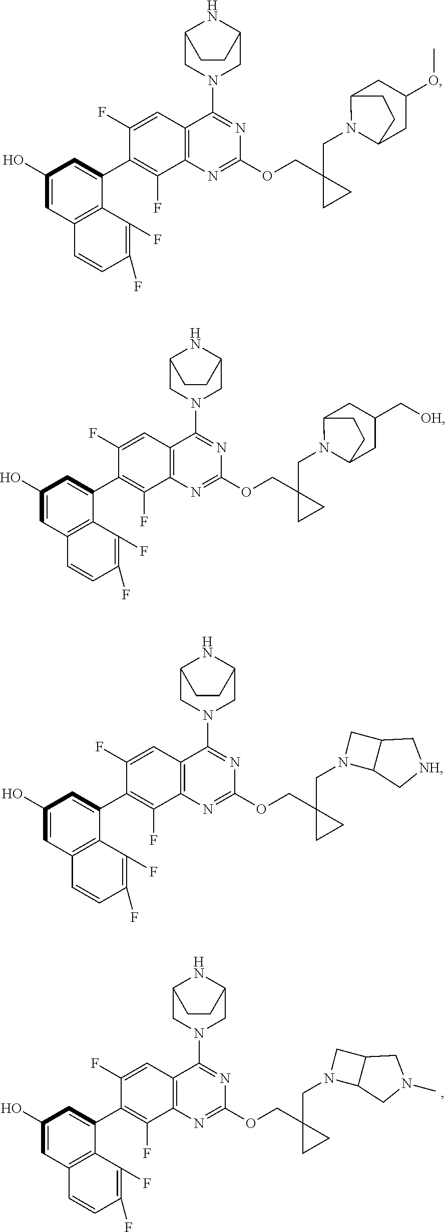

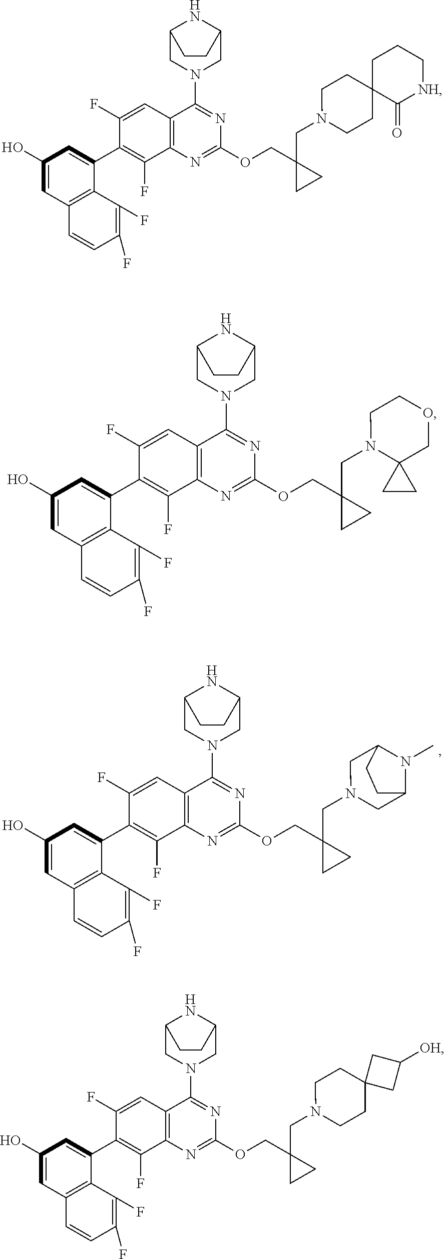

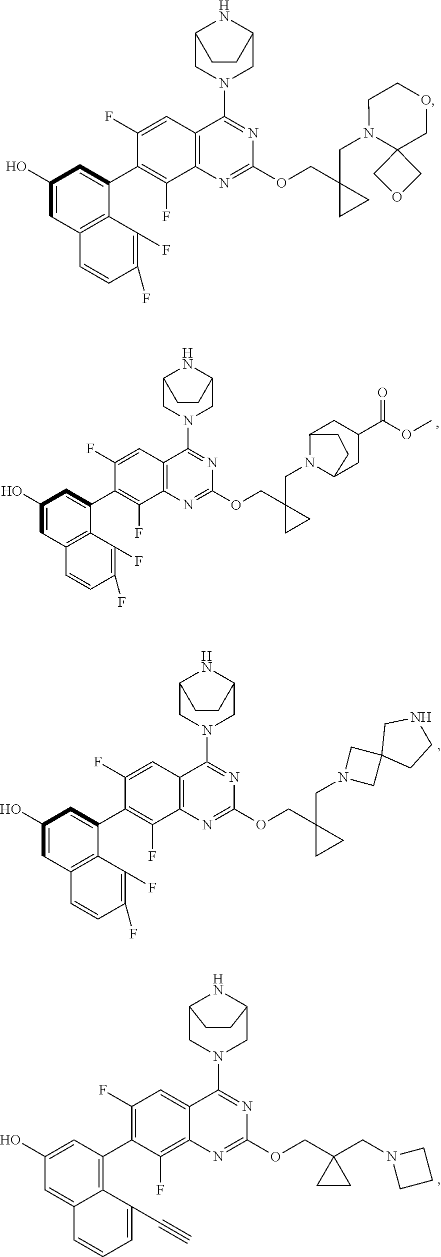

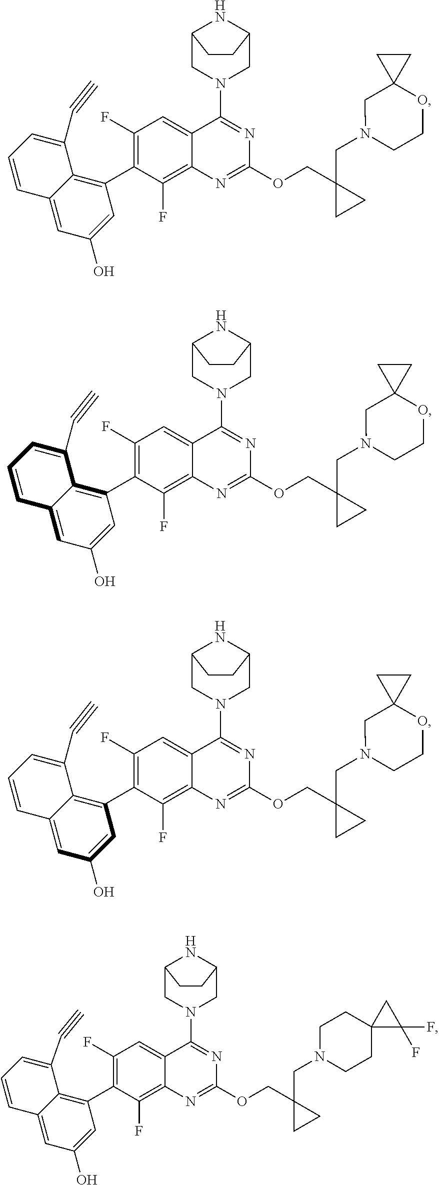

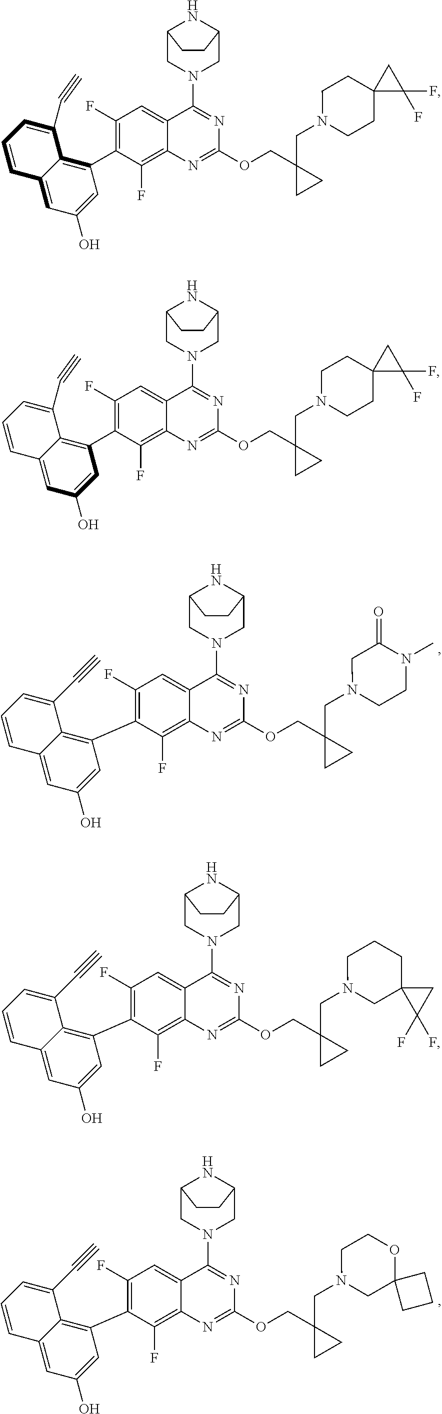

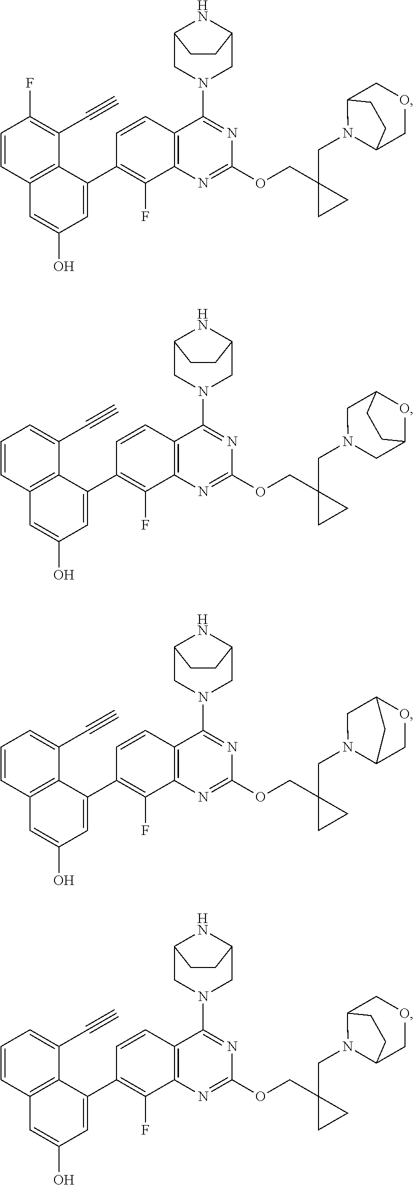 [SMILES: C#Cc1c(F)ccc2cc(O)cc(-c3ccc4c(N5CC6CCC(C5)N6)nc(OCC5(CN6C7CCC6COC7)CC5)nc4c3F)c12 ; C#Cc1cccc2cc(O)cc(-c3ccc4c(N5CC6CCC(C5)N6)nc(OCC5(CN6CC7CCC(C6)O7)CC5)nc4c3F)c12 ; C#Cc1cccc2cc(O)cc(-c3ccc4c(N5CC6CCC(C5)N6)nc(OCC5(CN6CC7CC6CO7)CC5)nc4c3F)c12 ; C#Cc1cccc2cc(O)cc(-c3ccc4c(N5CC6CCC(C5)N6)nc(OCC5(CN6C7CCC6COC7)CC5)nc4c3F)c12]

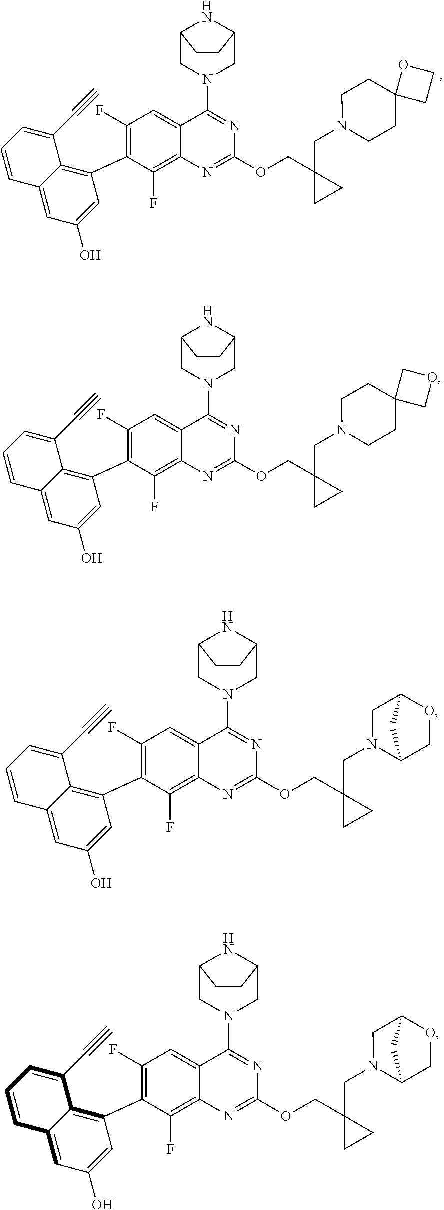

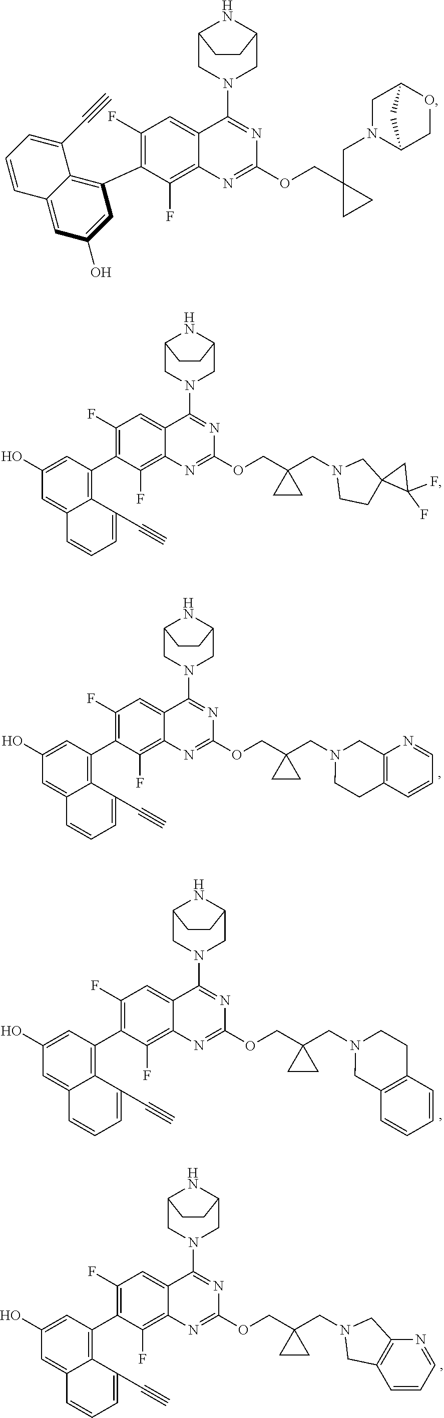

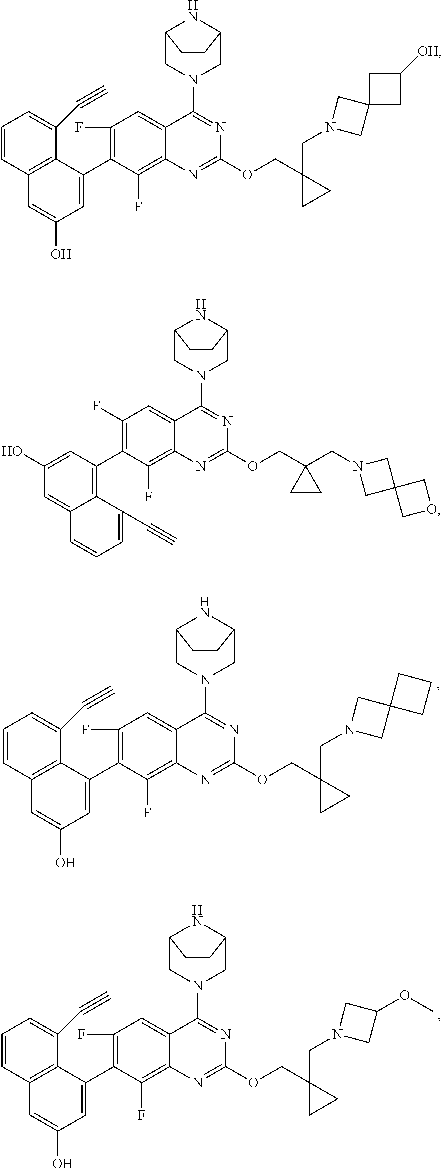 [SMILES: C#Cc1cccc2cc(O)cc(-c3c(F)cc4c(N5CC6CCC(C5)N6)nc(OCC5(CN6CC7(CC(O)C7)C6)CC5)nc4c3F)c12 ; C#Cc1cccc2cc(O)cc(-c3c(F)cc4c(N5CC6CCC(C5)N6)nc(OCC5(CN6CC7(COC7)C6)CC5)nc4c3F)c12 ; C#Cc1cccc2cc(O)cc(-c3c(F)cc4c(N5CC6CCC(C5)N6)nc(OCC5(CN6CC7(CCC7)C6)CC5)nc4c3F)c12 ; C#Cc1cccc2cc(O)cc(-c3c(F)cc4c(N5CC6CCC(C5)N6)nc(OCC5(CN6CC(OC)C6)CC5)nc4c3F)c12]

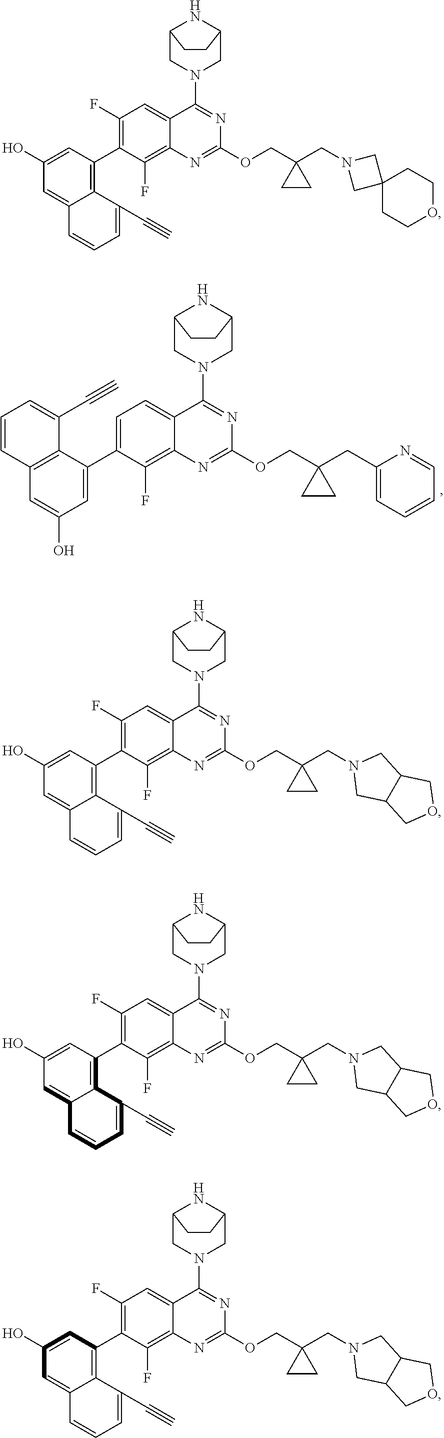

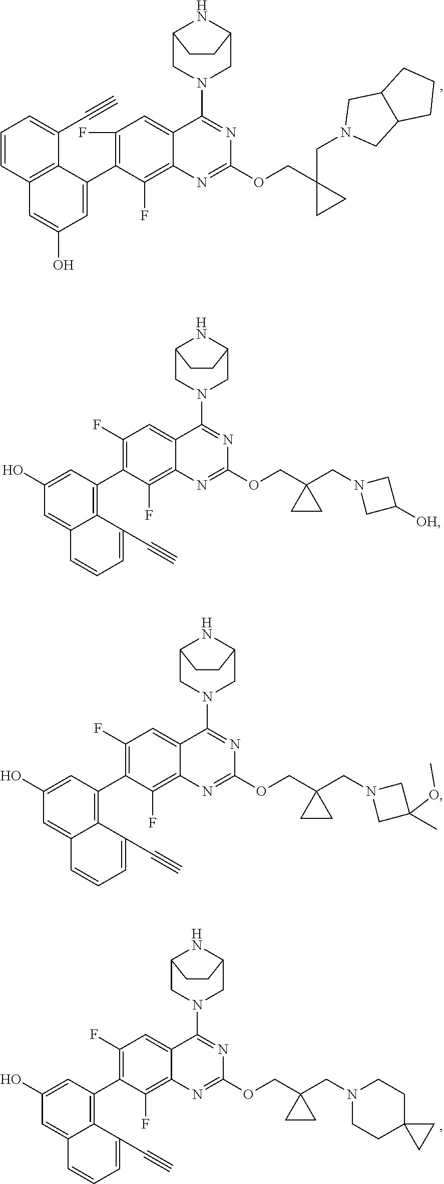 [SMILES: C#Cc1cccc2cc(O)cc(-c3c(F)cc4c(N5CC6CCC(C5)N6)nc(OCC5(CN6CC7CCCC7C6)CC5)nc4c3F)c12 ; C#Cc1cccc2cc(O)cc(-c3c(F)cc4c(N5CC6CCC(C5)N6)nc(OCC5(CN6CC(O)C6)CC5)nc4c3F)c12 ; C#Cc1cccc2cc(O)cc(-c3c(F)cc4c(N5CC6CCC(C5)N6)nc(OCC5(CN6CC(C)(OC)C6)CC5)nc4c3F)c12 ; C#Cc1cccc2cc(O)cc(-c3c(F)cc4c(N5CC6CCC(C5)N6)nc(OCC5(CN6CCC7(CC6)CC7)CC5)nc4c3F)c12]

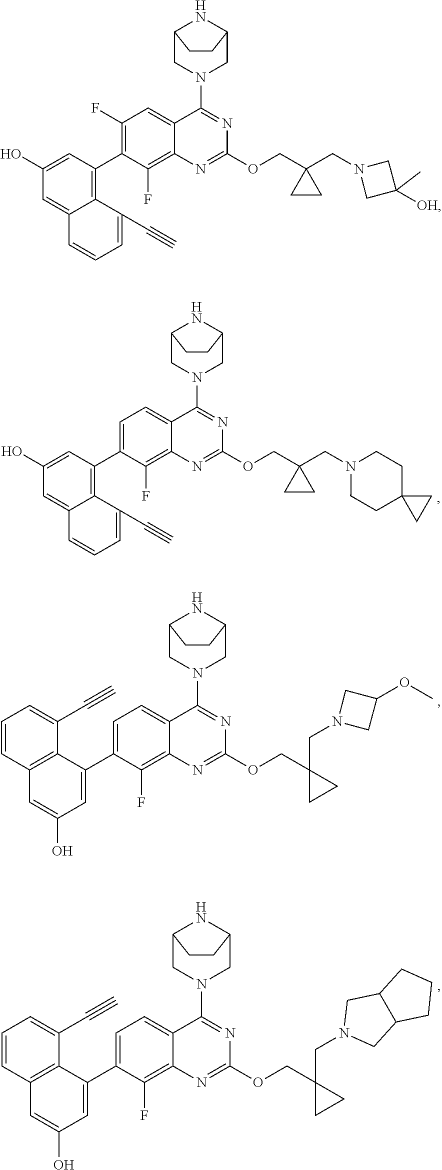 [SMILES: C#Cc1cccc2cc(O)cc(-c3c(F)cc4c(N5CC6CCC(C5)N6)nc(OCC5(CN6CC(C)(O)C6)CC5)nc4c3F)c12 ; C#Cc1cccc2cc(O)cc(-c3ccc4c(N5CC6CCC(C5)N6)nc(OCC5(CN6CCC7(CC6)CC7)CC5)nc4c3F)c12 ; C#Cc1cccc2cc(O)cc(-c3ccc4c(N5CC6CCC(C5)N6)nc(OCC5(CN6CC(OC)C6)CC5)nc4c3F)c12 ; C#Cc1cccc2cc(O)cc(-c3ccc4c(N5CC6CCC(C5)N6)nc(OCC5(CN6CC7CCCC7C6)CC5)nc4c3F)c12]

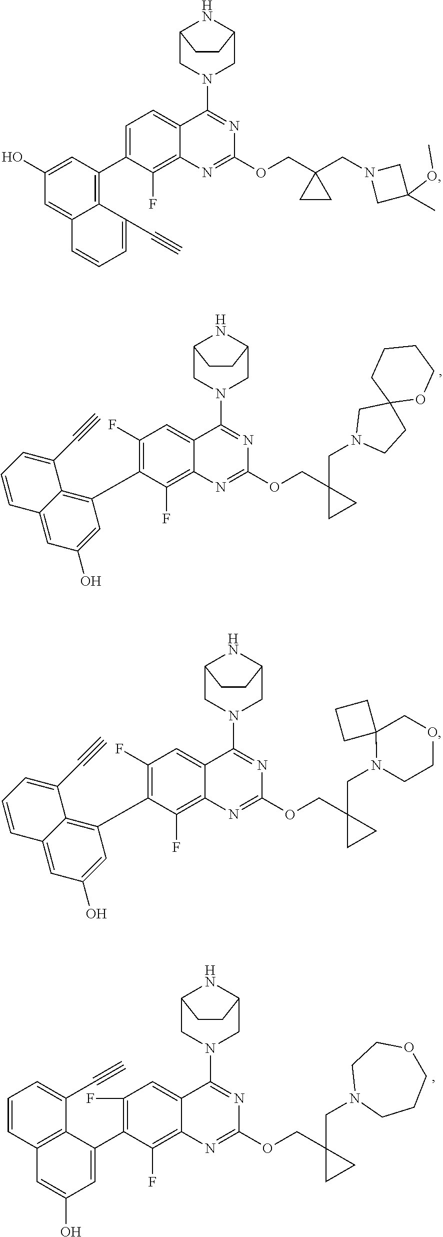 [SMILES: C#Cc1cccc2cc(O)cc(-c3ccc4c(N5CC6CCC(C5)N6)nc(OCC5(CN6CC(C)(OC)C6)CC5)nc4c3F)c12 ; C#Cc1cccc2cc(O)cc(-c3c(F)cc4c(N5CC6CCC(C5)N6)nc(OCC5(CN6CCC7(CCCCO7)C6)CC5)nc4c3F)c12 ; C#Cc1cccc2cc(O)cc(-c3c(F)cc4c(N5CC6CCC(C5)N6)nc(OCC5(CN6CCOCC67CCC7)CC5)nc4c3F)c12 ; C#Cc1cccc2cc(O)cc(-c3c(F)cc4c(N5CC6CCC(C5)N6)nc(OCC5(CN6CCCOCC6)CC5)nc4c3F)c12]

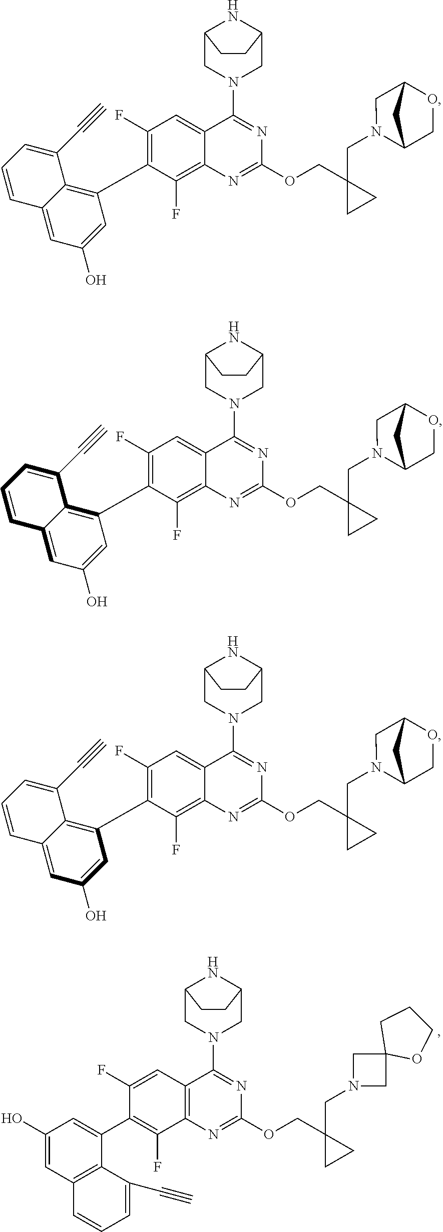

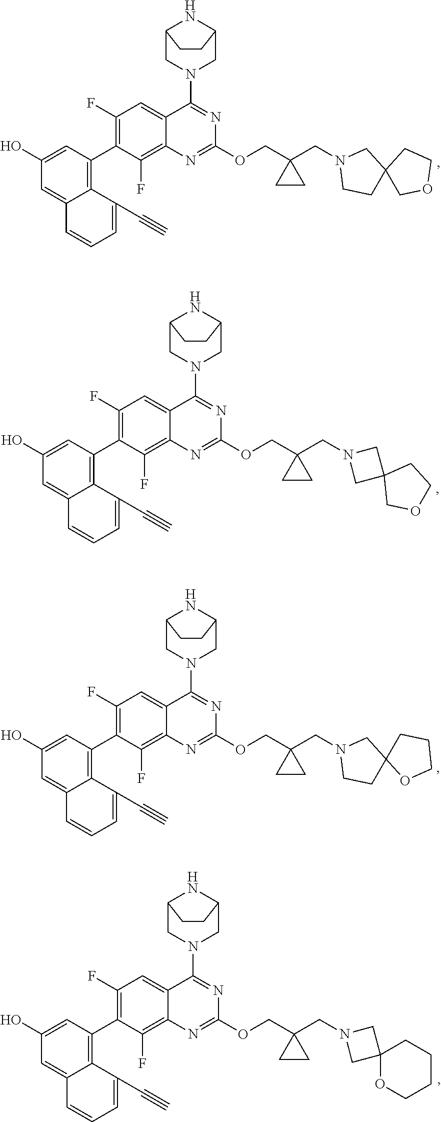 [SMILES: C#Cc1cccc2cc(O)cc(-c3c(F)cc4c(N5CC6CCC(C5)N6)nc(OCC5(CN6CCC7(CCOC7)C6)CC5)nc4c3F)c12 ; C#Cc1cccc2cc(O)cc(-c3c(F)cc4c(N5CC6CCC(C5)N6)nc(OCC5(CN6CC7(CCOC7)C6)CC5)nc4c3F)c12 ; C#Cc1cccc2cc(O)cc(-c3c(F)cc4c(N5CC6CCC(C5)N6)nc(OCC5(CN6CCC7(CCCO7)C6)CC5)nc4c3F)c12 ; C#Cc1cccc2cc(O)cc(-c3c(F)cc4c(N5CC6CCC(C5)N6)nc(OCC5(CN6CC7(CCCCO7)C6)CC5)nc4c3F)c12]

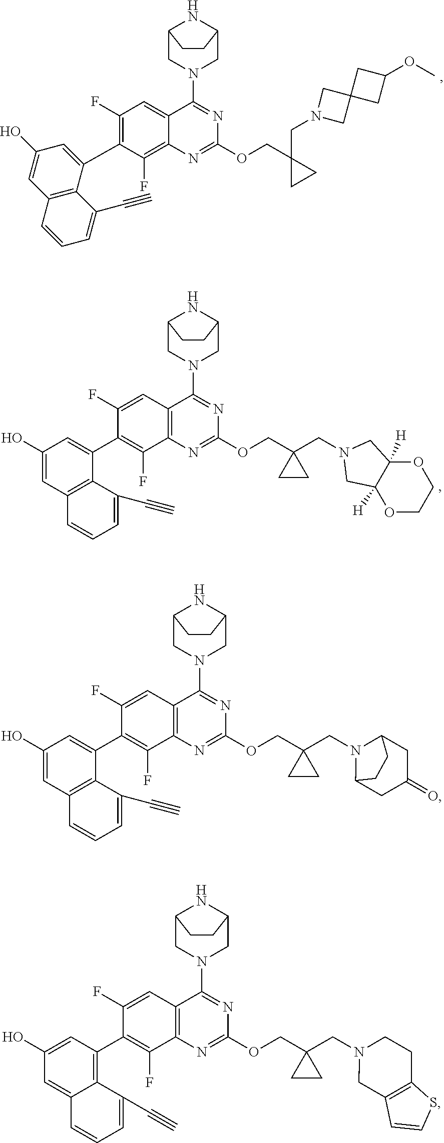 [SMILES: C#Cc1cccc2cc(O)cc(-c3c(F)cc4c(N5CC6CCC(C5)N6)nc(OCC5(CN6CC7(CC(OC)C7)C6)CC5)nc4c3F)c12 ; C#Cc1cccc2cc(O)cc(-c3c(F)cc4c(N5CC6CCC(C5)N6)nc(OCC5(CN6C[C@@H]7OCCO[C@@H]7C6)CC5)nc4c3F)c12 ; C#Cc1cccc2cc(O)cc(-c3c(F)cc4c(N5CC6CCC(C5)N6)nc(OCC5(CN6C7CCC6CC(=O)C7)CC5)nc4c3F)c12 ; C#Cc1cccc2cc(O)cc(-c3c(F)cc4c(N5CC6CCC(C5)N6)nc(OCC5(CN6CCc7sccc7C6)CC5)nc4c3F)c12]

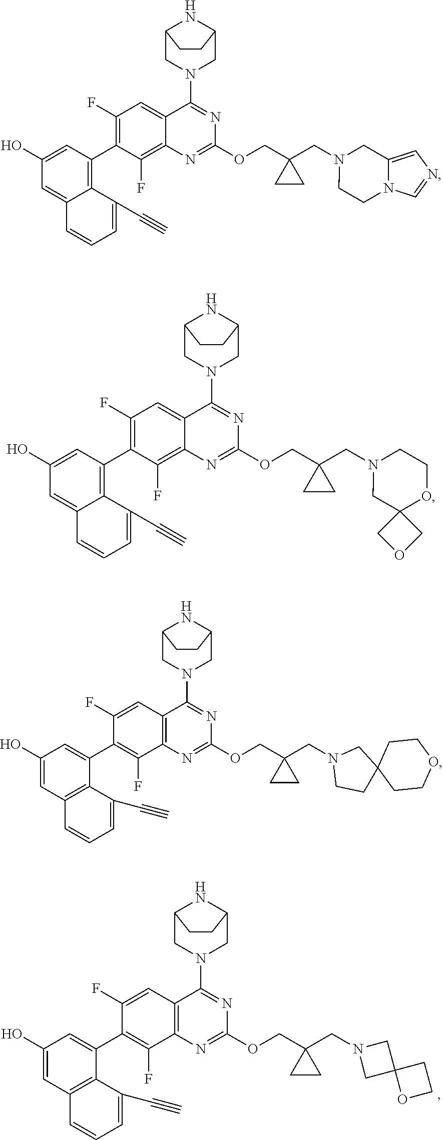 [SMILES: C#Cc1cccc2cc(O)cc(-c3c(F)cc4c(N5CC6CCC(C5)N6)nc(OCC5(CN6CCn7cncc7C6)CC5)nc4c3F)c12 ; C#Cc1cccc2cc(O)cc(-c3c(F)cc4c(N5CC6CCC(C5)N6)nc(OCC5(CN6CCOC7(COC7)C6)CC5)nc4c3F)c12 ; C#Cc1cccc2cc(O)cc(-c3c(F)cc4c(N5CC6CCC(C5)N6)nc(OCC5(CN6CCC7(CCOCC7)C6)CC5)nc4c3F)c12 ; C#Cc1cccc2cc(O)cc(-c3c(F)cc4c(N5CC6CCC(C5)N6)nc(OCC5(CN6CC7(CCO7)C6)CC5)nc4c3F)c12]

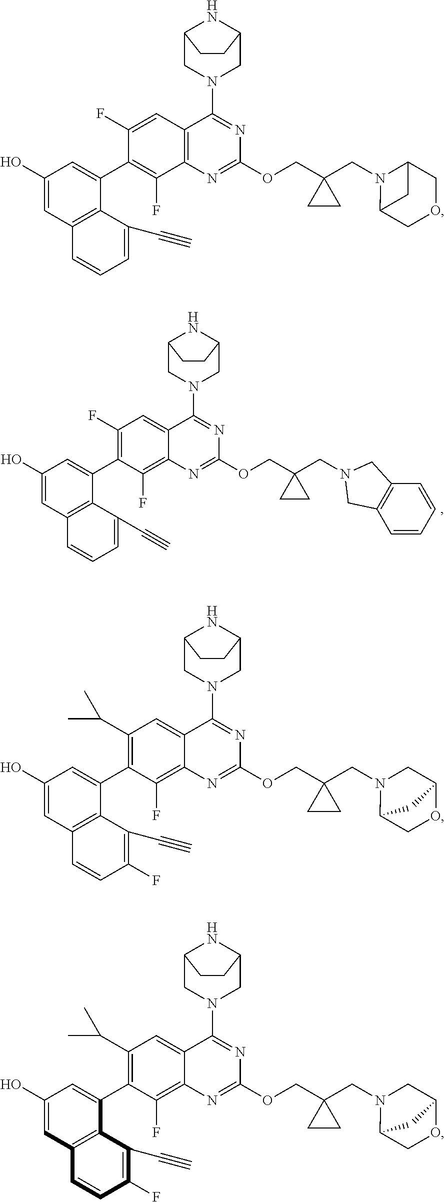

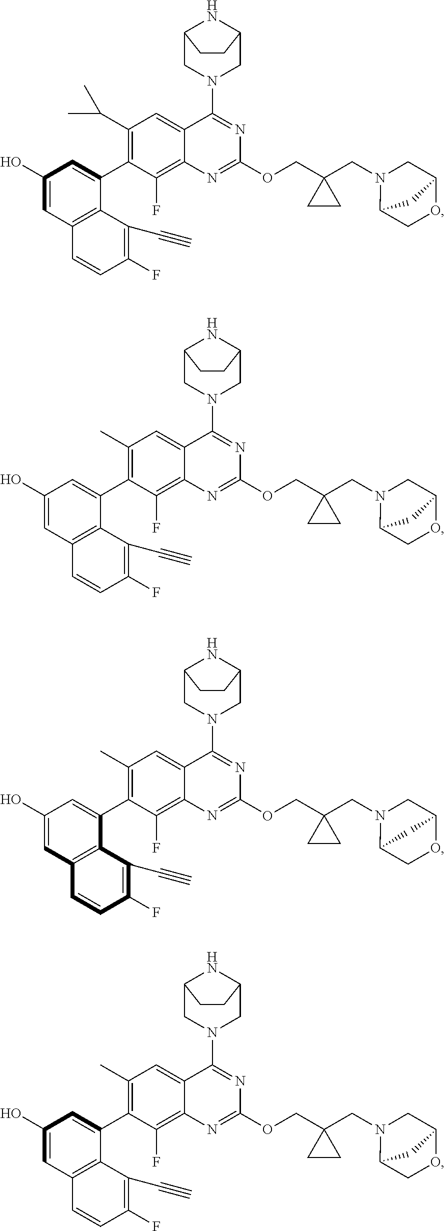

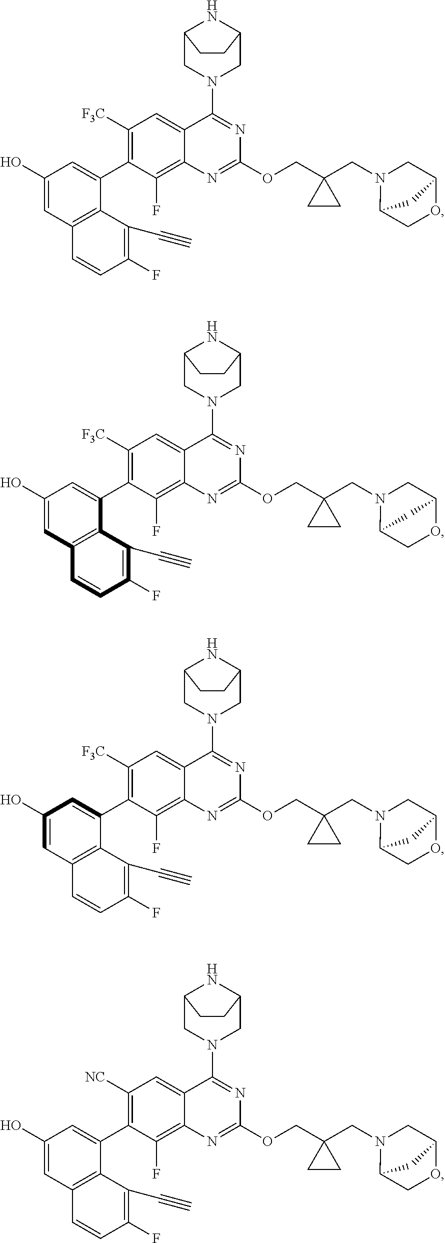

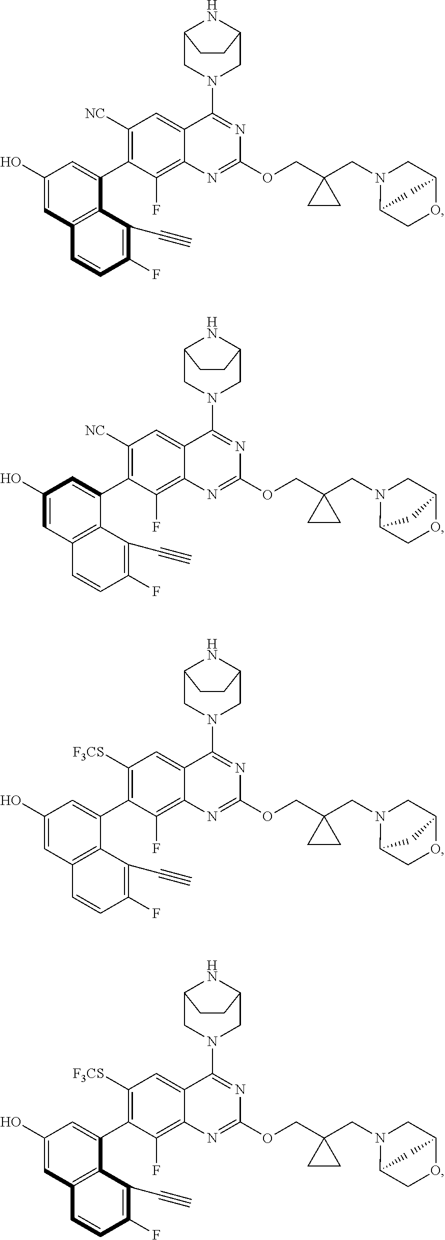

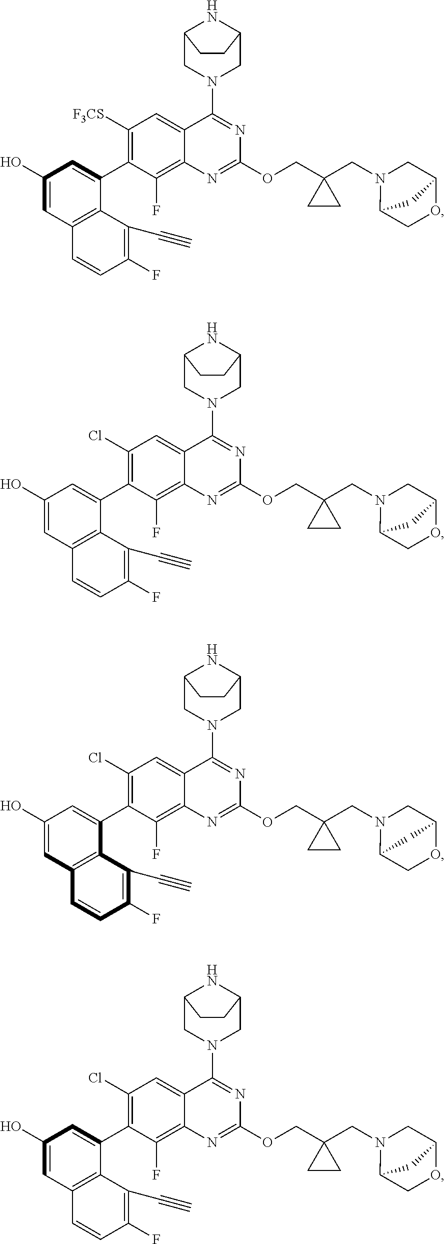

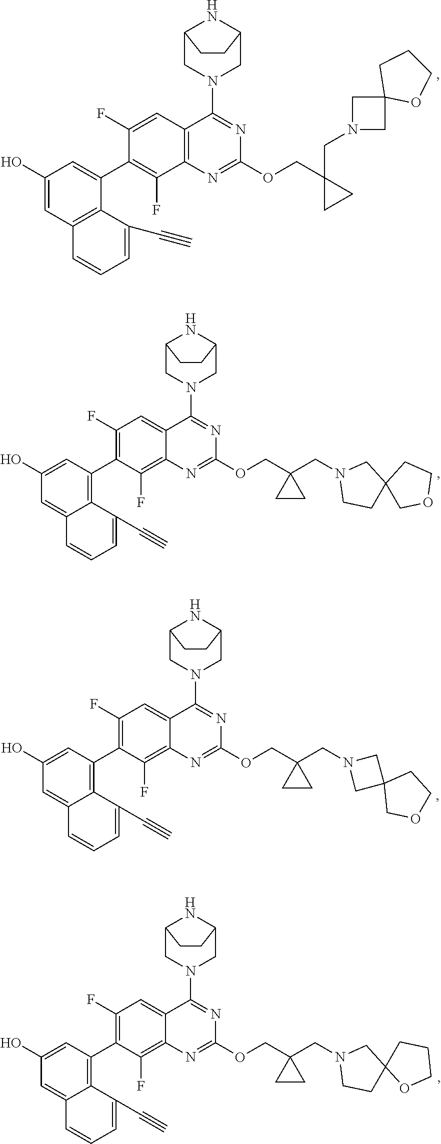 [SMILES: C#Cc1cccc2cc(O)cc(-c3c(F)cc4c(N5CC6CCC(C5)N6)nc(OCC5(CN6CC7(CCCO7)C6)CC5)nc4c3F)c12 ; C#Cc1cccc2cc(O)cc(-c3c(F)cc4c(N5CC6CCC(C5)N6)nc(OCC5(CN6CCC7(CCOC7)C6)CC5)nc4c3F)c12 ; C#Cc1cccc2cc(O)cc(-c3c(F)cc4c(N5CC6CCC(C5)N6)nc(OCC5(CN6CC7(CCOC7)C6)CC5)nc4c3F)c12 ; C#Cc1cccc2cc(O)cc(-c3c(F)cc4c(N5CC6CCC(C5)N6)nc(OCC5(CN6CCC7(CCCO7)C6)CC5)nc4c3F)c12]

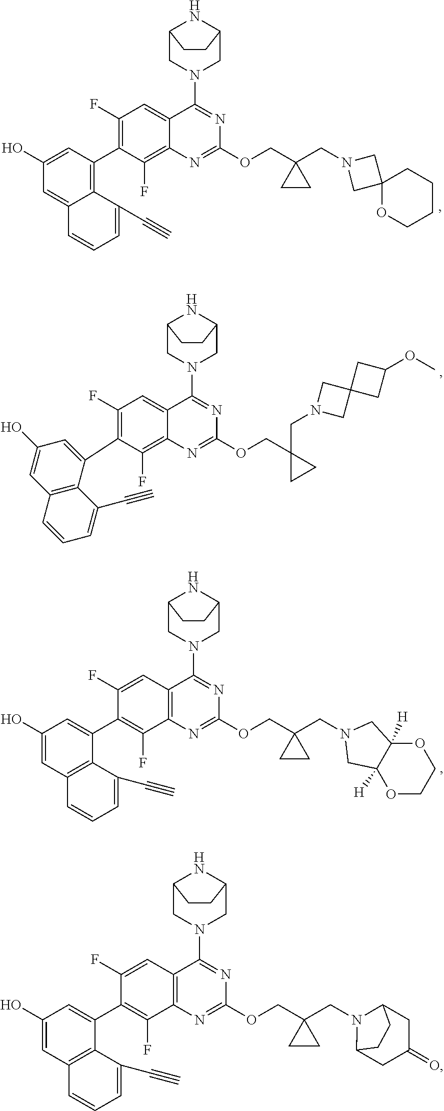 [SMILES: C#Cc1cccc2cc(O)cc(-c3c(F)cc4c(N5CC6CCC(C5)N6)nc(OCC5(CN6CC7(CCCCO7)C6)CC5)nc4c3F)c12 ; C#Cc1cccc2cc(O)cc(-c3c(F)cc4c(N5CC6CCC(C5)N6)nc(OCC5(CN6CC7(CC(OC)C7)C6)CC5)nc4c3F)c12 ; C#Cc1cccc2cc(O)cc(-c3c(F)cc4c(N5CC6CCC(C5)N6)nc(OCC5(CN6C[C@@H]7OCCO[C@@H]7C6)CC5)nc4c3F)c12 ; C#Cc1cccc2cc(O)cc(-c3c(F)cc4c(N5CC6CCC(C5)N6)nc(OCC5(CN6C7CCC6CC(=O)C7)CC5)nc4c3F)c12]

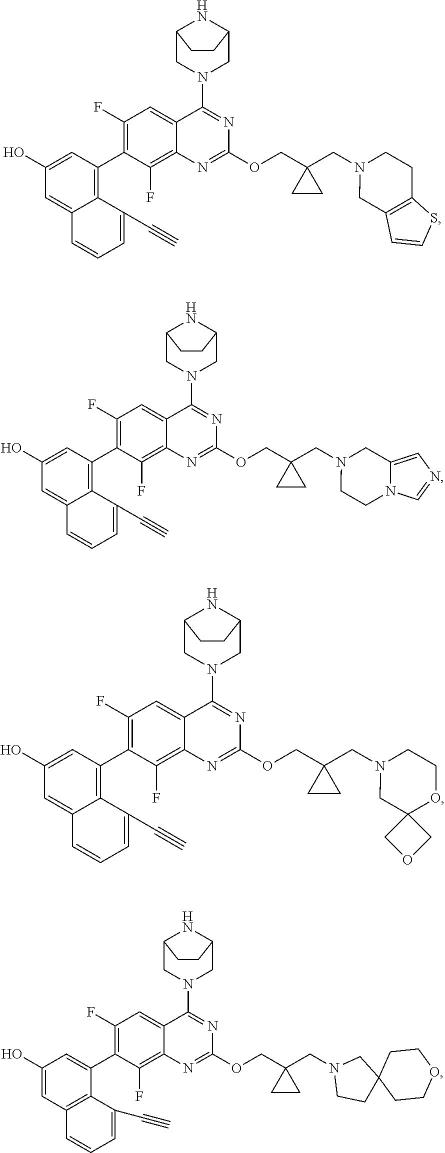 [SMILES: C#Cc1cccc2cc(O)cc(-c3c(F)cc4c(N5CC6CCC(C5)N6)nc(OCC5(CN6CCc7sccc7C6)CC5)nc4c3F)c12 ; C#Cc1cccc2cc(O)cc(-c3c(F)cc4c(N5CC6CCC(C5)N6)nc(OCC5(CN6CCn7cncc7C6)CC5)nc4c3F)c12 ; C#Cc1cccc2cc(O)cc(-c3c(F)cc4c(N5CC6CCC(C5)N6)nc(OCC5(CN6CCOC7(COC7)C6)CC5)nc4c3F)c12 ; C#Cc1cccc2cc(O)cc(-c3c(F)cc4c(N5CC6CCC(C5)N6)nc(OCC5(CN6CCC7(CCOCC7)C6)CC5)nc4c3F)c12]

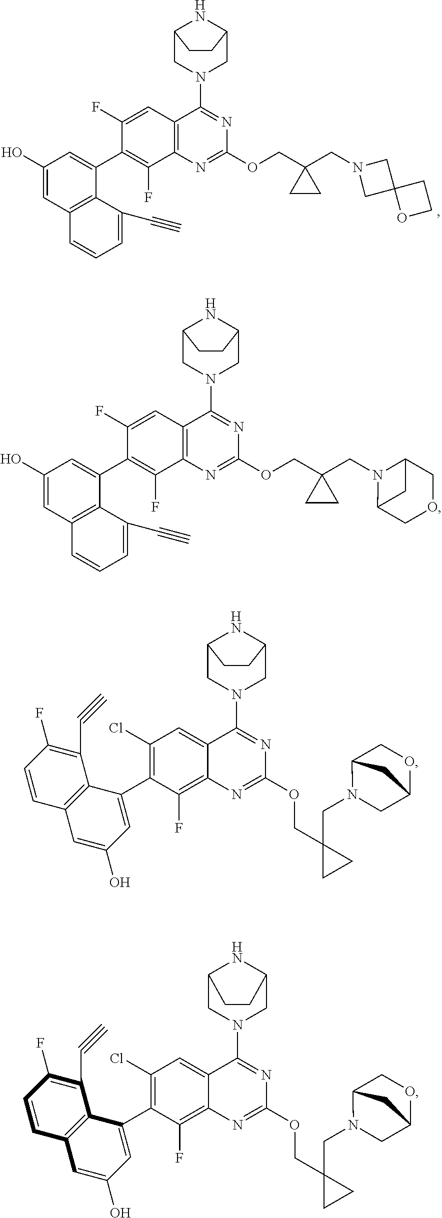

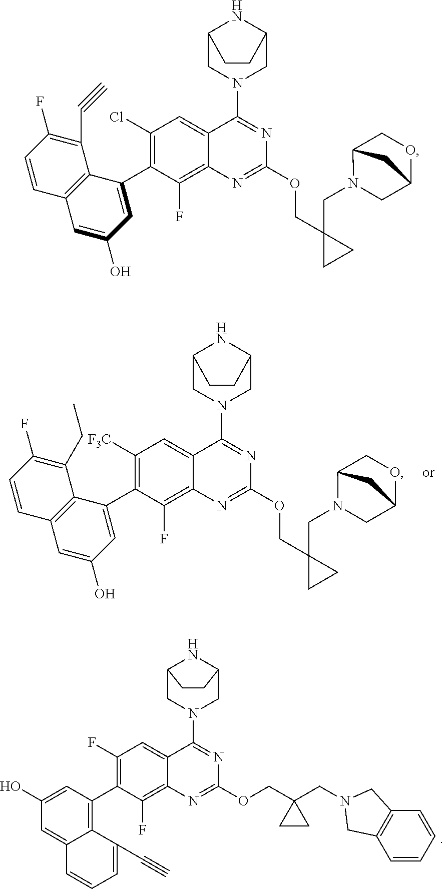

132. The compound according to claim 115, wherein, the compound is selected from any one of the following compounds:
An atropisomer, which is afforded by chiral separation of the compound having the structure 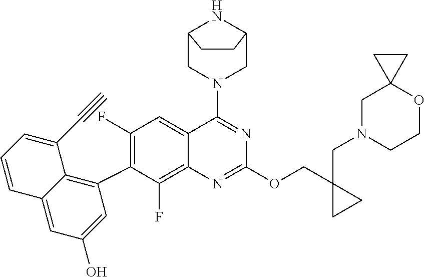 [SMILES: C#Cc1cccc2cc(O)cc(-c3c(F)cc4c(N5CC6CCC(C5)N6)nc(OCC5(CN6CCOC7(CC7)C6)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.266 min;
An atropisomer, which is afforded by chiral separation of the compound having the structure 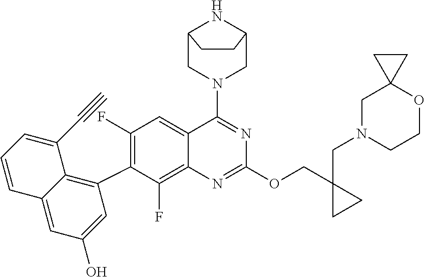 [SMILES: C#Cc1cccc2cc(O)cc(-c3c(F)cc4c(N5CC6CCC(C5)N6)nc(OCC5(CN6CCOC7(CC7)C6)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 6.685 min;
An atropisomer, which is afforded by chiral separation of the compound having the structure 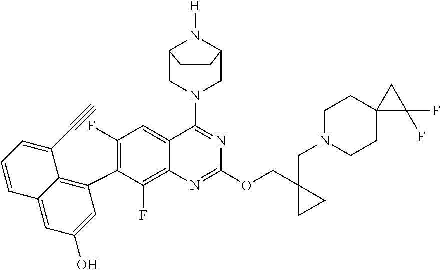 [SMILES: C#Cc1cccc2cc(O)cc(-c3c(F)cc4c(N5CC6CCC(C5)N6)nc(OCC5(CN6CCC7(CC6)CC7(F)F)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 3.665 min;
An atropisomer, which is afforded by chiral separation of the compound having the structure 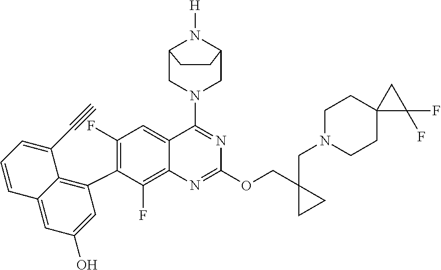 [SMILES: C#Cc1cccc2cc(O)cc(-c3c(F)cc4c(N5CC6CCC(C5)N6)nc(OCC5(CN6CCC7(CC6)CC7(F)F)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 5.532 min;
An atropisomer, which is afforded by chiral separation of the compound having the structure 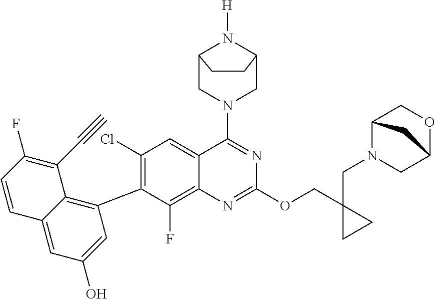 [SMILES: C#Cc1c(F)ccc2cc(O)cc(-c3c(Cl)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.561 min;
An atropisomer, which is afforded by chiral separation of the compound having the structure 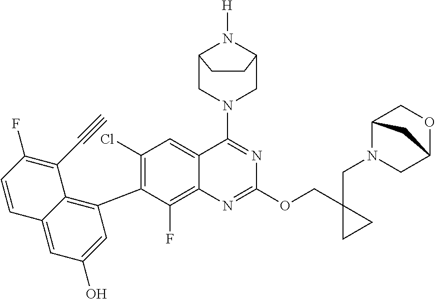 [SMILES: C#Cc1c(F)ccc2cc(O)cc(-c3c(Cl)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.002 min;
An atropisomer, which is afforded by chiral separation of the compound having the structure 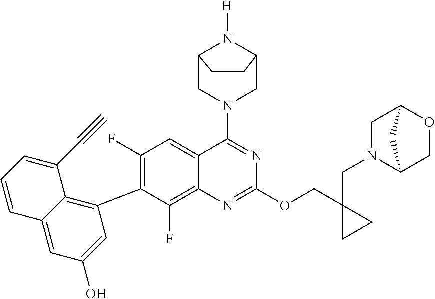 [SMILES: C#Cc1cccc2cc(O)cc(-c3c(F)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.297 min;
An atropisomer, which is afforded by chiral separation of the compound having the structure 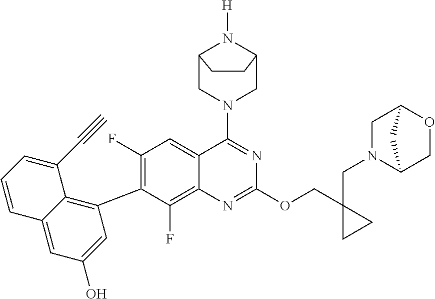 [SMILES: C#Cc1cccc2cc(O)cc(-c3c(F)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.337 min;
An atropisomer, which is afforded by chiral separation of the compound having the structure 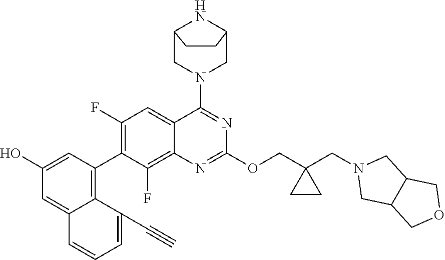 [SMILES: C#Cc1cccc2cc(O)cc(-c3c(F)cc4c(N5CC6CCC(C5)N6)nc(OCC5(CN6CC7COCC7C6)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.597 min;
An atropisomer, which is afforded by chiral separation of the compound having the structure 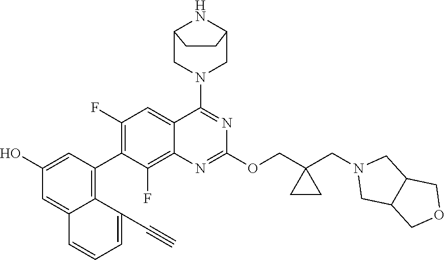 [SMILES: C#Cc1cccc2cc(O)cc(-c3c(F)cc4c(N5CC6CCC(C5)N6)nc(OCC5(CN6CC7COCC7C6)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 7.228 min;
An atropisomer, which is afforded by chiral separation of the compound having the structure 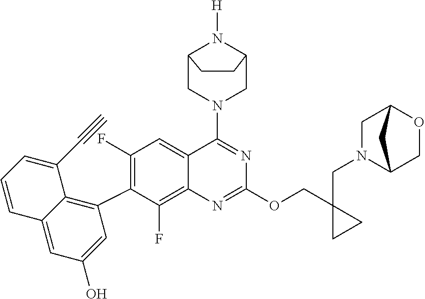 [SMILES: C#Cc1cccc2cc(O)cc(-c3c(F)cc4c(N5CC6CCC(C5)N6)nc(OCC5(CN6C[C@H]7C[C@@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 3.639 min;
An atropisomer, which is afforded by chiral separation of the compound having the structure 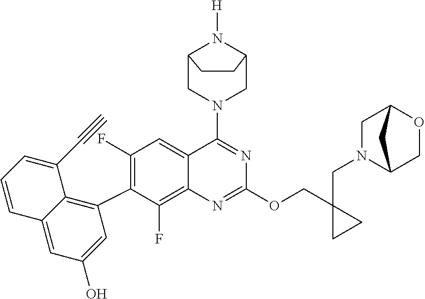 [SMILES: C#Cc1cccc2cc(O)cc(-c3c(F)cc4c(N5CC6CCC(C5)N6)nc(OCC5(CN6C[C@H]7C[C@@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 6.608 min;
An atropisomer, which is afforded by chiral separation of the compound having the structure 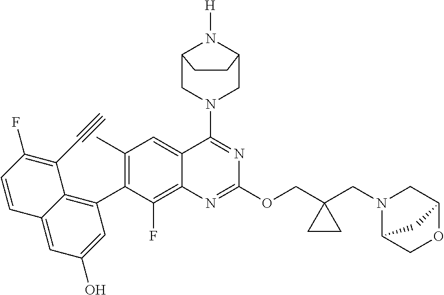 [SMILES: C#Cc1c(F)ccc2cc(O)cc(-c3c(C)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the first eluting isomer of the compound, preferably, the retention time of the first eluting isomer is about 4.217 min;
An atropisomer, which is afforded by chiral separation of the compound having the structure 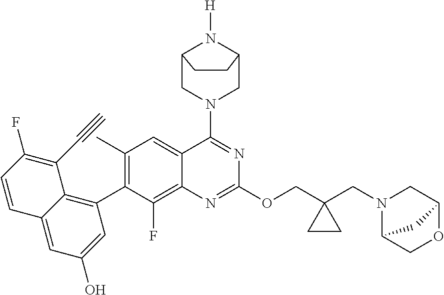 [SMILES: C#Cc1c(F)ccc2cc(O)cc(-c3c(C)cc4c(N5CC6CCC(C5)N6)nc(OCC5(CN6C[C@@H]7C[C@H]6CO7)CC5)nc4c3F)c12]
under the following conditions: instrument: Prep-HPLC-Gilson; chromatographic column: CHIRAL ART Amylose-SA column (2 cm×25 cm, 5 um); mobile phase: Hexane (0.1% isopropylamine)/EtOH (V/V=50:50); flow rate: 20 mL/min; wherein, the atropisomer is the second eluting isomer relative to another atropisomer of the compound, preferably, the retention time of the second eluting isomer is about 5.886 min.

133. A pharmaceutical composition comprising the compound of formula (I), a stereoisomer thereof, an atropisomer thereof, a deuterated derivative thereof, a tautomer thereof, a prodrug thereof, or a pharmaceutically acceptable salt thereof according to claim 115; and at least one pharmaceutically acceptable excipient.

134. A method of treating a subject having a diseases or conditions related to KRAS G12D protein, said method comprising administering to the subject a therapeutically effective amount of the compound of formula (I), a stereoisomer thereof, an atropisomer thereof, a deuterated derivative thereof, a tautomer thereof, a prodrug thereof, or a pharmaceutically acceptable salt thereof according to claim 115; wherein, the diseases or conditions related to KRAS G12D protein is cancer related to KRAS G12D protein; wherein, the cancer is selected from pancreatic cancer, colorectal cancer, endometrial cancer or lung cancer; wherein, the lung cancer is selected from non-small cell lung cancer or small cell lung cancer.
